(19) United States
(12) Patent Application Publication
(10) Pub. No.: US 2013/0158043 A1
(43) Pub. Date: Jun. 20, 2013

(54) PAK INHIBITORS FOR THE TREATMENT OF CANCER

(71) Applicant: Afraxis, Inc., La Jolla, CA (US)

(72) Inventors: David Campbell, San Diego, CA (US); Sergio G. Duron, San Diego, CA (US)

(73) Assignee: AFRAXIS, INC., La Jolla, CA (US)

(21) Appl. No.: 13/708,886

(22) Filed: Dec. 7, 2012

Related U.S. Application Data
(60) Provisional application No. 61/569,170, filed on Dec. 9, 2011.

(51) Int. Cl. C07D 471/04

(52) U.S. Cl. CPC C07D 471/04
USPC 514/252.16; 514/264.11

(57) ABSTRACT

Provided herein are methods of utilizing PAK inhibitors for the treatment of cancer. Further provided herein are compounds and formulations utilized for the treatment of cancer.

### CROSS-REFERENCE

This application claims the benefit of U.S. provisional application Ser. No. 61/569,170, filed Dec. 9, 2011 which is incorporated by reference in its entirety.

### BACKGROUND OF THE INVENTION

Cancer, also called malignancy, is characterized by an abnormal growth of cells. There are more than 100 types of cancer, including breast cancer, skin cancer, lung cancer, colon cancer, brain cancer, prostate cancer, kidney cancer, ovarian cancer, cancers of the central nervous system, leukemia, and lymphoma. Cancer symptoms vary widely based on the type of cancer. Cancer treatment includes chemotherapy, radiation, and surgery.

A number of cancers have been associated with alterations in the expression and/or activation of p21-activated kinases, which are central players in growth factor signaling networks and oncogenic processes that control cell proliferation, cell polarity, invasion and actin cytoskeleton organization.

The effects of cancer are devastating to the quality of life of those afflicted as well as that of their families. Moreover, cancer imposes an enormous health care burden on society.

### SUMMARY OF THE INVENTION

Described herein are compounds, compositions and methods for treating an individual suffering from a cell proliferative disorder, such as by way of example only, cancer (e.g., including breast cancer, skin cancer, lung cancer, colon cancer, brain cancer, prostate cancer, kidney cancer, liver cancer, cancer of the central nervous system, and lymphoma or the like) by administering to an individual a pharmaceutical composition comprising a therapeutically effective amount of an inhibitor of a p21-activated kinase (PAK), e.g., an inhibitor of PAK1, PAK2, PAK3, PAK4, PAK5, or PAK6, as described herein. The p21-activated kinase (PAK) family of serine/threonine kinases plays a pivotal role in physiological processes including motility, survival, mitosis, transcription and translation. PAKs are evolutionary conserved and widely expressed in a variety of tissues and are aberrantly expressed and/or activated in multiple cancer types. In some embodiments, inhibitors of one or more of Group I PAKs (PAK1, PAK2 and/or PAK3) and/or Group II PAKs (PAK-4, PAK5 and/or PAK6) are administered to inhibit aberrant cellular proliferation.

Provided herein are methods for treating a cell proliferative disorder in an individual in need thereof, comprising administering to the subject a therapeutically effective amount of a PAK inhibitor or a pharmaceutically acceptable salt, solvate or N-oxide thereof. Also provided herein, in some embodiments, are methods for treating a cell proliferative disorder, comprising administering to an individual in need thereof a therapeutically effective amount of a compound of Formula I-VI, or a pharmaceutically acceptable salt, solvate or N-oxide thereof, wherein compounds of Formula I-VI are as described herein.

Provided herein are pharmaceutical compositions comprising a therapeutically effective amount of a compound of Formula I-VI, or a pharmaceutically acceptable salt, solvate, or N-oxide thereof, and a pharmaceutically acceptable carrier, used for treating a cell proliferative disorder wherein the compound of Formula I-VI is as described herein.

Provided herein are pharmaceutical compositions comprising a therapeutically effective amount of a compound of Formula I-VI, or a pharmaceutically acceptable salt, solvate, or N-oxide thereof, and a pharmaceutically acceptable carrier, used for treating cancer wherein the compound of Formula I-VI is as described herein.

Provided herein is the use of compounds of any of Formula I-VI in the manufacture of a medicament for the treatment of a cell proliferative disorder. Also provided herein is the use of compounds of any of Formula I-VI in the manufacture of a medicament for the treatment of cancer.

Also provided herein, in some embodiments, are methods for treating cancer, such as by way of example only, breast cancer, colorectal cancer, brain cancer, lung cancer, neurofibromatosis, pancreatic cancer, kidney cancer, liver cancer, cancer of the central nervous system, liver cancer, skin cancer, comprising administering to an individual in need thereof a therapeutically effective amount of a compound of Formula I-VI, or a pharmaceutically acceptable salt, solvate, or N-oxide thereof, wherein compounds of Formula I-VI are as described herein. In some embodiments, the cancer is a recurrent cancer. In some embodiments, the cancer is a refractory cancer.

In some embodiments, the cancer is selected from ovarian, breast, colon, brain, chronic myelogenous leukemia, renal cell carcinoma, gastric, leukemia, lung, melanoma, prostate, T-cell lymphoma, heptocellular, bladder, kidney, glioblastoma, mesothelioma, neuroma, meningioma, neuroblastoma, medulloblastoma, peripheral malignant nerve sheath tumor, ependymoma, chraniopharyngioma, astrocytoma, germinoma, glioma, mixed glioma, choroid plexus tumor, oligodendroglioma, peripheral neuroectodermal tumor, primitive neuroectodermal tumor (PNET), CNS lymphoma, pituitary adenoma, schwannoma, head and neck cancer, and esophageal cancer. In some embodiments, the cancer is selected from NSCLC, SCLC, or mesothelioma. In some embodiments, the cancer is an ovarian cancer. In some embodiments, the kidney cancer is a renal cell carcinoma. In some embodiments, the cancer is a meningioma. In some embodiments, the cancer is a head and neck cancer. In some embodiments, the cancer is an esophageal cancer. In some embodiments, the esophageal cancer is an esophageal squamous cancer. In some embodiments, the cancer is a breast cancer. In some embodiments, the cancer is a colorectal cancer. In some embodiments, the cancer is a schwannoma. In some embodiments, the schwannoma is a bilateral vestibular schwannoma.

Also provided herein, in some embodiments, are methods for treating a subject suffering from a cancer of the central nervous system comprising administering to the subject a therapeutically effective amount of a compound of Formula I-VI, or a pharmaceutically acceptable salt, solvate, or N-oxide thereof, wherein compounds of Formula I-VI are as described herein.

In some embodiments, the cancer of the central nervous system is a tumor associated with neurofibromatosis type 1 or neurofibromatosis type 2. In some embodiments, the tumor associated with neurofibromatosis type 1 or neurofibromatosis type 2 is a neurofibroma, optic glioma, malignant peripheral nerve sheath tumor, schwannoma, ependymoma, or meningioma. In some embodiments, the schwannoma is a bilateral vestibular schwannoma.

In some embodiments, the cancer is a recurrent cancer. In some embodiments, the cancer is a refractory cancer. In some embodiments, the cancer is a malignant cancer. In some embodiments, is a method for treating a non-malignant tumor.

Also provided herein, in some embodiments, are methods for treating a subject suffering from a cancer of the nervous system comprising administering to the subject a therapeutically effective amount of a compound of Formula I-VI, or a pharmaceutically acceptable salt, solvate, or N-oxide thereof, wherein compounds of Formula I-VI are as described herein.

In some embodiments, the cancer of the nervous system is a tumor of the peripheral nervous system.

Provided herein, in some embodiments, are methods of treating cancer comprising modulating a p21-activated kinase by contacting a p21-activated kinase with a compound of Formula I-VI.

In some embodiments of any of the above methods, compounds of any of Formula I-VI are inhibitors of p21-activated kinase. In some embodiments, compounds of any of Formula I-VI inhibit one or more of PAK1, PAK2, PAK3, PAK-4, PAK5 or PAK6. In some embodiments of any of the above methods, compounds of any of Formula I-VI inhibit one or more of PAK1, PAK2 or PAK3. In some embodiments of any of the above methods, compounds of any of Formula I-VI inhibit PAK1 and PAK3. In some embodiments of any of the above methods, compounds of any of Formula I-VI inhibit PAK1 and PAK2. In some embodiments of any of the above methods, compounds of any of Formula I-VI inhibit PAK1, PAK2 and PAK3. In some embodiments of any of the above methods, compounds of any of Formula I-VI inhibit PAK1 and PAK-4. In some embodiments of any of the above methods, compounds of any of Formula I-V1 inhibit PAK1, PAK2, PAK3 and PAK-4.

In some embodiments of any of the above methods, compounds of any of Formula I-VI inhibit PAK1. In some embodiments of any of the above methods, compounds of any of Formula I-VI inhibit PAK2. In some embodiments of any of the above methods, compounds of any of Formula I-VI inhibit PAK3. In some embodiments of any of the above methods, compounds of any of Formula I-VI inhibit PAK-4.

In some embodiments of any of the above methods, a therapeutically effective amount of compounds of any of Formula I-VI causes substantially complete inhibition of one or more Group I p21-activated kinases.

In some embodiments of any of the above methods, a therapeutically effective amount of compounds of any of Formula I-VI causes partial inhibition of one or more Group I p21-activated kinases.

In some embodiments of any of the above methods, the method further comprises administration of a second therapeutic agent that alleviates one or more symptoms associated with a cell proliferative disorder. In some embodiments, the second therapeutic agent is an anti-cancer agent. In some embodiments, the anti-cancer agent is a pro-apoptotic agent or a kinase inhibitor. In some embodiments, the anti-cancer agent is a pro-apoptotic agent, a kinase inhibitor, or a receptor tyrosine kinase inhibitor. In some embodiments, the pro-apoptotic agent is an antagonist of inhibitor of apoptosis (IAP) proteins. In some embodiments, the antagonist of IAP proteins is BV6 or G-416. In some embodiments, the kinase inhibitor is a receptor tyrosine kinase (RTK) inhibitor, non-receptor tyrosine kinase (non-RTK) inhibitor, or a serine/threonine kinase inhibitor. In some embodiments, the kinase inhibitor is a RTK inhibitor selected from a group comprising an EGFR inhibitor, PDGFR inhibitor, FGFR inhibitor, VEGFR inhibitor, and HGFR inhibitor. In some embodiments, the RTK inhibitor is an EGFR inhibitor selected from a group comprising afatinib, lapatinib, neratinib, erlotinib, neratinib, vandetanib, and gefitinib. In some embodiments, the RTK inhibitor is an PDGFR inhibitor selected from a group comprising axitinib, pazopanib, sorafenib and MP470. In some embodiments, the RTK inhibitor is an FGFR inhibitor selected from a group comprising ponatinib, AZD4547, PD173074, TKI-258, and SU5402. In some embodiments, the RTK inhibitor is an VEGFR inhibitor selected from a group comprising axitinib, AZD2171, pazopanib, regorafenib, semaxanib, sorafenib, tivozanib, foretinib, and vandetanib. In some embodiments, the RTK inhibitor is an HGFR inhibitor selected from a group comprising PHA-665752, crizotinib, PF-02341066, K252a, SU11274, ARQ197, foretinib, SGX523, and MP470. In some embodiments, the kinase inhibitor is a MAPK inhibitor. In some embodiments, the MAPK inhibitor is a RAF inhibitor, MEK inhibitor, ERK inhibitor, or any combination thereof. In some embodiments, the MAPK inhibitor is selected from a group comprising VX-702, JIP-1(153-163), VX-745, LY2228820, vinorelbine, and BIRB796. In some embodiments, the MAPK inhibitor is an ERK inhibitor selected from a group comprising sorafenib, GDC-0879, and BIX 02189. In some embodiments, the MAPK inhibitor is a MEK inhibitor selected from a group comprising AZD6244, CI-1040, PD0325901, RDEA119, U0126-EtOH, PD98059, AS703026, PD318088, AZD8330, TAK-733, and GSK1120212. In some embodiments, the MAPK inhibitor is a RAF inhibitor selected from a group comprising RAF265, GDC-0879, PLX-4720, regorafenib, PLX4032, SB590885, and ZM336372. In some embodiments, the kinase inhibitor is a PI3K/AKT/mTOR inhibitor selected from a group comprising rapamycin, CCI-779, everolimus, NVP-BEZ235, PI-103, temsirolimus, AZD8055, KU-0063794, PF-04691502, CH132799, RG7422, palomid 529, PP242, XL765, GSK1059615, PKI-587, WAY-600, WYE-687, WYE-125132, and WYE-354.

In some embodiments, the anti-cancer therapeutic agent is selected from the group comprising chemotherapy, radiation therapy, surgery, immunotherapy, and monoclonal antibody therapy.

Provided herein are methods of treating cancer comprising inhibiting the activity of one or more p21-activated kinases by contacting the one or more p21-activated kinases with a compound of any of Formula I-VI. In some embodiments, the one or more p21-activated kinase is contacted with a compound of any of Formula I-VI in vitro. In some embodiments, the one or more p21-activated kinase is contacted with a compound of any of Formula I-VI in vivo.

As used herein, compounds of any of Formula I-VI used in the treatment of cancer includes compounds of Formula I, Formula Ia, compounds of Formula II, compounds of Formula IIa, compounds of Formula IIb, compounds of Formula IIc, compounds of Formula IId, compounds of Formula IIe, compounds of Formula IIf, compounds of Formula IIg, compounds of Formula IIh, compounds of Formula IIi, compounds of Formula IIj, compounds of Formula IIk, compounds of Formula IIm, compounds of Formula IIn, compounds of Formula IIo, compounds of Formula IIp, compounds of Formula IIq, compounds of Formula IIr, compounds of Formula IIs, compounds of Formula IIt, compounds of Formula IIu, compounds of Formula III, compounds of Formula IV, compounds of Formula IVa, compounds of Formula V, compounds of Formula Va, compounds of Formula Vb, compounds of Formula Vc, compounds of Formula Vd, compounds of Formula Ve, compounds of Formula Vf, compounds of Formula Vg, compounds of Formula Vh, or compounds of Formula VI, or pharmaceutically acceptable salt, solvate or N-oxide thereof.

### DETAILED DESCRIPTION OF THE INVENTION

Provided herein are compounds and formulations utilized for the treatment of one or more diseases or disorders characterized by aberrant cell proliferation. In some embodiments, the disease or disorder characterized by aberrant cell proliferation is a cancer. In some embodiments, the cancer is a malignant cancer. In some embodiments, the cancer is a solid tumor. In some embodiments, the solid tumor is a sarcoma or carcinoma. In some embodiments, the cancer is a leukemia or lymphoma. In some embodiments, the cancer is a recurrent cancer. In some embodiments, the cancer is a refractory cancer.

A cancer is an abnormal growth of cells (usually derived from a single cell). The cells have lost normal control mechanisms and thus are able to expand continuously, invade adjacent tissues, migrate to distant parts of the body, and promote the growth of new blood vessels from which the cells derive nutrients. As used herein, a cancer can be malignant or benign. Cancer can develop from any tissue within the body. As cells grow and multiply, they form a mass of tissue, called a tumor. The term tumor refers to an abnormal growth or mass. Tumors can be cancerous (malignant) or noncancerous (benign). Cancerous tumors can invade neighboring tissues and spread throughout the body (metastasize). Benign tumors, however, do not invade neighboring tissues and do not spread throughout the body. Cancer can be divided into those of the blood and blood-forming tissues (leukemias and lymphomas) and “solid” tumors. “Solid” tumors can be carcinomas or sarcomas.

In some embodiments, the cancer is a leukemia or a lymphoma. In some embodiments, the cancer is a leukemia. Leukemias are cancers of white blood cells or of cells that develop into white blood cells. White blood cells develop from stem cells in the bone marrow. Sometimes the development goes awry, and pieces of chromosomes get rearranged. The resulting abnormal chromosomes interfere with normal control of cell division, so that affected cells multiply uncontrollably and become cancerous (malignant), resulting in leukemia. Leukemia cells ultimately occupy the bone marrow, replacing or suppressing the function of cells that develop into normal blood cells. This interference with normal bone marrow cell function can lead to inadequate numbers of red blood cells (causing anemia), white blood cells (increasing the risk of infection), and platelets (increasing the risk of bleeding). Leukemia cells may also invade other organs, including the liver, spleen, lymph nodes, testes, and brain. Leukemias are grouped into four main types: acute lymphocytic leukemia, acute myelocytic leukemia, chronic lymphocytic leukemia, and chronic myelocytic leukemia. The types are defined according to how quickly they progress and the type and characteristics of the white blood cells that become cancerous. Acute leukemias progress rapidly and consist of immature cells. Chronic leukemias progress slowly and consist of more mature cells. Lymphocytic leukemias develop from cancerous changes in lymphocytes or in cells that normally produce lymphocytes. Myelocytic (myeloid) leukemias develop from cancerous changes in cells that normally produce neutrophils, basophils, eosinophils, and monocytes. Additional types of leukemias include hairy cell leukemia, chronic myelomonocytic leukemia, and juvenile myelomonocytic-leukemia.

In some embodiments, the cancer is a lymphoma. Lymphomas are cancers of the lymphocytes, which reside in the lymphatic system and in blood-forming organs. Lymphomas are cancers of a specific type of white blood cell known as lymphocytes. These cells help fight infections. Lymphomas can develop from either B or T lymphocytes. T lymphocytes are important in regulating the immune system and in fighting viral infections. B lymphocytes produce antibodies. Lymphocytes move about to all parts of the body through the bloodstream and through a network of tubular channels called lymphatic vessels. Scattered throughout the network of lymphatic vessels are lymph nodes, which house collections of lymphocytes. Lymphocytes that become cancerous (lymphoma cells) may remain confined to a single lymph node or may spread to the bone marrow, the spleen, or virtually any other organ. The two major types of lymphoma are Hodgkin lymphoma, previously known as Hodgkin's disease, and non-Hodgkin lymphoma. Non-Hodgkin lymphomas are more common than Hodgkin lymphoma. Burkitt's lymphoma and mycosis fungoides are subtypes of non-Hodgkin lymphomas. Hodgkin lymphoma is marked by the presence of the Reed-Sternberg cell. Non-Hodgkin lymphomas are all lymphomas which are not Hodgkin's lymphoma. Non-Hodgkin lymphomas can be further divided into indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

In some embodiments, the cancer is a solid tumor. In some embodiments, the solid tumor is a sarcoma or carcinoma. In some embodiments, the solid tumor is a sarcoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma). In some embodiments, the cancer is a schwannoma. In some embodiments, the schwannoma is a spontaneous schwannoma. In some embodiments, the schwannoma is a malignant scwhannoma. In some embodiments, the schwannoma is a bilateral vestibular scwhannoma.

In some embodiments, the solid tumor is a carcinoma. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some embodiments, the cancer is a breast cancer. In some embodiments, the cancer is an ovarian cancer. In some embodiments, the cancer is a head and neck cancer. In some embodiments, the cancer is an esophageal cancer. In some embodiments, the cancer is an esophageal squamous cancer.

In some embodiments, the cancer is a kidney cancer. In some embodiments, the kidney cancer is a renal cell carcinoma.

In some embodiments, the cancer is a skin cancer. In some embodiments, the skin cancer is a basal cell carcinoma. Basal cell carcinomas account for about more than 90% of all skin cancers. Basal cell carcinomas are generally slow-growing and seldom spread. In some instances, basal cell carcinomas can spread and invade bone and other tissues under the skin. In some embodiments, the skin cancer is a squamous cell carcinoma. Squamous cell carcinomas can be more aggressive than basal cell carcinomas. In some instances, squamous cell carcinomas are more likely to grow deep below the skin and spread to distant parts of the body. These types of skin cancer sometimes are called nonmelanoma skin cancer. In some embodiments, the skin cancer is an actinic (solar) keratosis. An actinic keratosis is a precancerous condition that can develop into squamous cell carcinoma. In some instances, actinic keratosis appears as rough, red or brown, scaly patches on the skin. In some instances, they are often more easily felt than seen. In some instances, actinic keratosis is found on sun-exposed areas of the body, but it can be found on other parts of the body as well. In some instances, the skin cancer is a melanoma. A melanoma is a cancer that begins in the cells that produce skin pigment.

In some embodiments, the cancer is a lung cancer. In some embodiments the lung cancer is mesotheliomia. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. NSCLC account for about 85 to 87% of lung cancers. NSCLC grows more slowly than small cell lung carcinoma. Nevertheless, by the time about 40% of people are diagnosed, the cancer has spread to other parts of the body outside of the chest. Examples of NSCLC include squamous cell carcinoma, adenocarcinoma, and large cell carcinoma. Small cell lung carcinoma, also called oat cell carcinoma, accounts for about 13 to 15% of all lung cancers. It is very aggressive and spreads quickly. By the time that most people are diagnosed, the cancer has metastasized to other parts of the body. Malignant mesothelioma is an uncommon cancerous tumor of the lining of the lung and chest cavity (pleura) or lining of the abdomen (peritoneum) that is typically due to long-term asbestos exposure.

In some embodiments, the cancer is a CNS tumor. CNS tumors may be classified as gliomas or nongliomas. In some embodiments, the cancer is a glioma. In some instances, the glioma is a malignant glioma. In some embodiments, the glioma is a high grade glioma. In some embodiments, the glioma is a diffuse intrinsic pontine glioma. In some embodiments, the cancer is a nonglioma. Nongliomas include meningiomas, pituitary adenomas, primary CNS lymphomas, and medulloblastomas. In some embodiments, the cancer is a meningioma.

In some embodiments, the cancer is a brain cancer. In some embodiments, the brain cancer is a glioblastoma.

In some instances, the cancer is a glioma. Examples of gliomas include astrocytomas, oligodendrogliomas (or mixtures of oligodendroglioma and astrocytoma elements), and ependymomas. In some embodiments, the cancer is an astrocytoma. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Glioblastoma multiforme is the most common and most malignant of the primary brain tumors. Although this tumor can occur in all age groups, including children, the average age at which it is diagnosed is 55 years. The onset of symptoms is often abrupt and is most commonly related to mass effect and focal neurologic symptoms. Seizures are also relatively common. Intracranial bleeding may be the presenting symptom in less than 3% of patients. The duration of symptoms before diagnosis is usually short, ranging from a few days to a few weeks.

In some embodiments, the cancer is an oligodendroglioma. Oligodendrogliomas include low-grade oligodendrogliomas (or oligoastrocytomas) and anaplastic oligodendriogliomas.

In some embodiments, the cancer of the CNS is a tumor associated with neurofibromatosis (NF). In some embodiments, the neurofibromatosis is a type 1 NF or a type 2 NF. As used herein, NF includes Type 1 NF and Type 2 NF. In some embodiments, the neurofibromatosis is a type 1 NF. Neurofibromatosis type 1 is a condition characterized by changes in skin coloring (pigmentation) and the growth of tumors along nerves in the skin, brain, and other parts of the body. The signs and symptoms of this condition vary widely among affected people.

Beginning in early childhood, almost all people with neurofibromatosis type 1 have multiple café-au-lait spots, which are flat patches on the skin that are darker than the surrounding area. These spots increase in size and number as the individual grows older. Freckles in the underarms and groin typically develop later in childhood.

Most adults with neurofibromatosis type 1 develop neurofibromas, which are noncancerous (benign) tumors that are usually located on or just under the skin. These tumors may also occur in nerves near the spinal cord or along nerves elsewhere in the body. Some people with neurofibromatosis type 1 develop cancerous tumors that grow along nerves. These tumors, which usually develop in adolescence or adulthood, are called malignant peripheral nerve sheath tumors. People with neurofibromatosis type 1 also have an increased risk of developing other cancers, including brain tumors and cancer of blood-forming tissue (leukemia). In some embodiments, the cancer is a neurofibroma. Neurofibromas include dermal and plexiform neurofibromas. In some embodiments, the cancer is a malignant peripheral nerve sheath tumor (MPNST).

During childhood, benign growths called Lisch nodules often appear in the colored part of the eye (the iris). Lisch nodules do not interfere with vision. Some affected individuals also develop tumors that grow along the nerve leading from the eye to the brain (the optic nerve). These tumors, which are called optic gliomas, may lead to reduced vision or total vision loss. In some cases, optic gliomas have no effect on vision. In some embodiments, the cancer is an optic glioma.

In some embodiments, the cancer of the CNS is a tumor associated with neurofibromatosis. In some embodiments, the neurofibromatosis is a type 2 NF. Neurofibromatosis type 2 is a disorder characterized by the growth of noncancerous tumors in the nervous system. The tumors associated with neurofibromatosis type 2 include cranial and spinal tumors, including, but not limited to, bilateral vestibular schwannomas, acoustic neuromas, ependyomomas, or meningiomas. These growths develop in the brain or along the nerve that carries information from the inner ear to the brain (the auditory nerve). In some embodiments, the cancer is bilateral vestibular schwannoma, acoustic neuroma, ependyomoma, or meningioma.

The signs and symptoms of this condition usually appear during adolescence or in a person's early twenties, although onset can occur at any age. The most frequent early symptoms of vestibular schwannomas are hearing loss, ringing in the ears (tinnitus), and problems with balance. In most cases, these tumors occur in both ears by age 30. If tumors develop in other parts of the brain or spinal cord, signs and symptoms vary according to their location. Complications of tumor growth can include changes in vision or sensation, numbness or weakness in the arms or legs, fluid buildup in the brain, and nerve compression leading to significant morbidities and death. Some people with neurofibromatosis type 2 also develop clouding of the lens (cataracts) in one or both eyes, often beginning in childhood.

In some embodiments, the cancer is characterized by aberrant NF1 gene expression or activity. In some embodiments, the cancer is characterized by a reduction in NF1 gene expression or activity. In some embodiments, NF1 gene expression or activity is reduced at least about 10%, at least about 20%, at least about 30%, at least about 40%, at least about 50%, at least about 60%. In other embodiments, NF1 gene expression or activity is reduced at least about 70%, at least about 75%, at least about 80%, or at least about 85%. Preferably, NF1 gene expression or activity is reduced at least about 90%, at least about 95%, at least about 97%, at least about 98%, or at least about 99%. In some embodiments, the cancer is characterized by a mutation in the NF1 gene.

In some embodiments, any of the cancers disclosed herein are characterized by aberrant NF2 gene expression or activity. In some embodiments, the cancer is characterized by a reduction in NF2 gene expression or activity. In some embodiments, NF2 gene expression or activity is reduced at least about 10%, at least about 20%, at least about 30%, at least about 40%, at least about 50%, at least about 60%. In other embodiments, NF2 gene expression or activity is reduced at least about 70%, at least about 75%, at least about 80%, or at least about 85%. Preferably, NF2 gene expression or activity is reduced at least about 90%, at least about 95%, at least about 97%, at least about 98%, or at least about 99%. In some embodiments, the cancer is characterized by a mutation in the NF2 gene. In some instances, modulation of PAK activity (e.g., inhibition or partial inhibition of PAK) alleviates, reverses or reduces the incidence of tumor development associated with NF.

p21-Activated Kinases (PAKs)

The PAKs constitute a family of serine-threonine kinases that is composed of “conventional”, or Group I PAKs, that includes PAK1, PAK2, and PAK3, and “non-conventional”, or Group II PAKs, that includes PAK-4, PAK5, and PAK6. See, e.g., Zhao et al. (2005), Biochem J, 386:201-214. These kinases function downstream of the small GTPases Rac and/or Cdc42 to regulate multiple cellular functions, including dendritic morphogenesis and maintenance (see, e.g., Ethell et at (2005), Prog in Neurobiol, 75:161-205; Penzes et al (2003), Neuron, 37:263-274), motility, morphogenesis, angiogenesis, and apoptosis, (see, e.g., Bokoch et al., 2003, Annu. Rev. Biochem., 72:743; and Hofmann et al., 2004, J. Cell Sci., 117:4343). GTP-bound Rac and/or Cdc42 bind to inactive PAK, releasing steric constraints imposed by a PAK autoinhibitory domain and/or permitting PAK phosphorylation and/or activation. Numerous phosphorylation sites have been identified that serve as markers for activated PAK.

In some instances, upstream effectors of PAK include, but are not limited to, TrkB receptors; NMDA receptors; adenosine receptors; estrogen receptors; integrins, EphB receptors; CDK5, FMRP; Rho-family GTPases, including Cdc42, Rac (including but not limited to Rac1 and Rac2), Chp, TC10, and Wrnch-1; guanine nucleotide exchange factors (“GEFs”), such as but not limited to GEFT, α-p-21-activated kinase interacting exchange factor (αPIX), Kalirin-7, and Tiam1; G protein-coupled receptor kinase-interacting protein 1 (GIT1), and sphingosine.

In some instances, downstream effectors of PAK include, but are not limited to, substrates of PAK kinase, such as Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), Ras, Raf, Mek, p47phox, BAD, caspase 3, estrogen and/or progesterone receptors, RhoGEF, GEF-H1, NET1, Gaz, phosphoglycerate mutase-B, RhoGDI, prolactin, p41Arc, cortactin and/or Aurora-A (See, e.g., Bokoch et al., 2003, Annu. Rev. Biochem., 72:743; and Hofmann et al., 2004, J. Cell Sci., 117:4343). Other substances that bind to PAK in cells include CIB; sphingolipids; lysophosphatidic acid, G-protein β and/or γ subunits; PIX/COOL; GIT/PKL; Nef; Paxillin; NESH; SH3-containing proteins (e.g. Nck and/or Grb2); kinases (e.g. Akt, PDK1, PI 3-kinase/p85, Cdk5, Cdc2, Src kinases, Abl, and/or protein kinase A (PKA)); and/or phosphatases (e.g. phosphatase PP2A, POPX1, and/or POPX2).

### PAK Inhibitors

Described herein are PAK inhibitors that treat one or more symptoms associated with cell proliferative disorders. Also described herein are pharmaceutical compositions comprising a PAK inhibitor (e.g., a PAK inhibitor compound described herein) for reversing or reducing one or more of tumor development and/or metastasis and/or negative symptoms and/or positive symptoms associated with cell proliferative disorders. Also described herein are pharmaceutical compositions comprising a PAK inhibitor (e.g., a PAK inhibitor compound described herein) for halting or delaying the progression of tumor development and/or metastasis and/or negative symptoms and/or positive symptoms associated with cell proliferative disorders. Described herein is the use of a PAK inhibitor for manufacture of a medicament for treatment of one or more symptoms of a cell proliferative disorder.

In some embodiments, the PAK inhibitor is a Group I PAK inhibitor that inhibits, for example, one or more Group I PAK polypeptides, for example, PAK1, PAK2, and/or PAK3. In some embodiments, the PAK inhibitor is a PAK1 inhibitor. In some embodiments, the PAK inhibitor is a PAK2 inhibitor. In some embodiments, the PAK inhibitor is a PAK3 inhibitor. In some embodiments, the PAK inhibitor is a mixed PAK1/PAK3 inhibitor. In some embodiments, the PAK inhibitor is a mixed PAK1/PAK2 inhibitor. In some embodiments, the PAK inhibitor is a mixed PAK1/PAK-4 inhibitor. In some embodiments, the PAK inhibitor is a mixed PAK1/PAK2/PAK-4 inhibitor. In some embodiments, the PAK inhibitor is a mixed PAK1/PAK2/PAK3/PAK-4 inhibitor. In some embodiments, the PAK inhibitor inhibits all three Group I PAK isoforms (PAK1, 2 and PAK3) with equal or similar potency. In some embodiments, the PAK inhibitor is a Group II PAK inhibitor that inhibits one or more Group II PAK polypeptides, for example PAK-4, PAK5, and/or PAK6. In some embodiments, the PAK inhibitor is a PAK-4 inhibitor. In some embodiments, the PAK inhibitor is a PAK5 inhibitor. In some embodiments, the PAK inhibitor is a PAK6 inhibitor.

In certain embodiments, a PAK inhibitor described herein reduces or inhibits the activity of one or more of PAK1, PAK2, PAK3, and/or PAK-4 while not affecting the activity of PAK5 and PAK6. In some embodiments, a PAK inhibitor described herein reduces or inhibits the activity of one or more of PAK1, PAK2 and/or PAK3 while not affecting the activity of PAK-4, PAK5 and/or PAK6. In some embodiments, a PAK inhibitor described herein reduces or inhibits the activity of one or more of PAK1, PAK2, PAK3, and/or one or more of PAK-4, PAK5 and/or PAK6. In some embodiments, a PAK inhibitor described herein is a substantially complete inhibitor of one or more PAKs. As used herein, “substantially complete inhibition” means, for example, >95% inhibition of one or more targeted PAKs. In other embodiments, “substantially complete inhibition” means, for example, >90% inhibition of one or more targeted PAKs. In some other embodiments, “substantially complete inhibition” means, for example, >80% inhibition of one or more targeted PAKs. In some embodiments, a PAK inhibitor described herein is a partial inhibitor of one or more PAKs. As used herein, “partial inhibition” means, for example, between about 40% to about 60% inhibition of one or more targeted PAKs. In other embodiments, “partial inhibition” means, for example, between about 50% to about 70% inhibition of one or more targeted PAKs. As used herein, where a PAK inhibitor substantially inhibits or partially inhibits the activity of a certain PAK isoform while not affecting the activity of another isoform, it means, for example, less than about 10% inhibition of the non-affected isoform when the isoform is contacted with the same concentration of the PAK inhibitor as the other substantially inhibited or partially inhibited isoforms. In other instances, where a PAK inhibitor substantially inhibits or partially inhibits the activity of a certain PAK isoform while not affecting the activity of another isoform, it means, for example, less than about 5% inhibition of the non-affected isoform when the isoform is contacted with the same concentration of the PAK inhibitor as the other substantially inhibited or partially inhibited isoforms. In yet other instances, where a PAK inhibitor substantially inhibits or partially inhibits the activity of a certain PAK isoform while not affecting the activity of another isoform, it means, for example, less than about 1% inhibition of the non-affected isoform when the isoform is contacted with the same concentration of the PAK inhibitor as the other substantially inhibited or partially inhibited isoforms.

In some embodiments are PAK inhibitors suitable for treating cancer having the structure of Formula I:

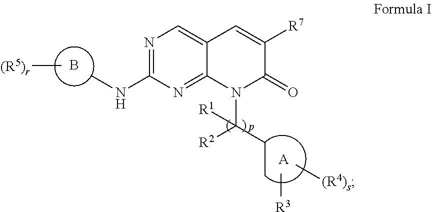

wherein:
R¹ and R² are each independently H or substituted or unsubstituted alkyl; or R¹ and R² together with the carbon to which they are attached form a substituted or unsubstituted C₃-C₆ cycloalkyl ring;
p is 1, 2 or 3;
ring A is heteroaryl comprising 1-4 heteroatoms selected from O, S and N;
R³ is a substituted or unsubstituted cycloalkyl, substituted or unsubstituted heteroaryl attached to ring A via a carbon atom, or substituted or unsubstituted heterocycloalkyl attached to ring A via a carbon atom;
each R⁴ is independently halogen, —CN, —NO₂, —OH, —OCF₃, —OCHF₂, —OCF₂H, —CF₃, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
R⁸ is H or substituted or unsubstituted alkyl, substituted or unsubstituted cycloalkyl, substituted or unsubstituted heterocycloalkyl, substituted or unsubstituted aryl or substituted or unsubstituted heteroaryl;
R⁹ is substituted or unsubstituted alkyl, substituted or unsubstituted cycloalkyl, substituted or unsubstituted heterocycloalkyl, substituted or unsubstituted aryl or substituted or unsubstituted heteroaryl;
each R¹⁰ is independently H, substituted or unsubstituted alkyl, substituted or unsubstituted cycloalkyl, substituted or unsubstituted heterocycloalkyl substituted or unsubstituted aryl or substituted or unsubstituted heteroaryl, or two R¹⁰ together with the atoms to which they are attached form a substituted or unsubstituted heterocycle;
s is 0-4;
ring B is aryl or heteroaryl;
each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8; and
R⁷ is H, halogen, —CN, substituted or unsubstituted alkyl, —C(═O)N(R¹⁰)₂, —CO₂R¹⁰, —OR¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
or a pharmaceutically acceptable salt, solvate or N-oxide thereof.

In one embodiment is a compound of Formula I wherein ring A is a 5-10-membered heteroaryl ring comprising 0-4 N atoms, 0-2 O atoms, 0-2 S atoms, or any combination thereof; wherein at least one N, S, or O atom is present.

In another embodiment, is a compound of Formula I wherein ring A is pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, or pyrazine. In one embodiment, ring A is pyridine. In another embodiment, ring A is imidazole. In a further embodiment, ring A is oxazole. In yet another embodiment, ring A is pyrazole. In yet another embodiment, ring A is furan. In yet another embodiment, ring A is pyrimidine. In yet another embodiment, ring A is pyrazine.

In one embodiment is a compound of Formula I wherein R¹ and R² are each independently H. In another embodiment, is a compound of Formula I wherein p is 1. In a further embodiment, is a compound of Formula I wherein p is 2. In another embodiment is a compound of Formula I wherein R¹ is methyl and R² is H.

In yet another embodiment, is a compound of Formula I, wherein ring B is an aryl ring. In another embodiment, ring B is a substituted or unsubstituted phenyl. In a further embodiment, ring B is a substituted or unsubstituted naphthalene. In a further embodiment, is a compound of Formula I, wherein ring B is a heteroaryl ring selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, and pyrazine.

In yet a further embodiment, is a compound of Formula I, wherein R³ is a C₃-C₆ cycloalkyl ring; a 5-6-membered heteroaryl ring comprising 1-3 N atoms, an O atom, a S atom, or any combination thereof; or a 3-6-membered heterocycloalkyl ring comprising 1-3 N atoms, an O atom, a S atom; or any combination thereof, and wherein R³ is further substituted by halogen, —CN, —NO₂, —OH, —SR⁸, —S(═O)R⁹, —S(═O)₂R⁹, NR¹⁰S(═O)₂R⁹, —S(═O)₂N(R¹⁰)₂, —(═O)R⁹, —OC(═O)R⁹, —CO₂R¹⁰, —N(R¹⁰)₂, —C(═O)N(R¹⁰)₂, C(O)R¹⁰, —NR¹⁰C(O)OR¹⁰, —NR¹⁰C(═O)N(R¹⁰)₂, —OR¹⁰, substituted or unsubstituted alkyl, substituted or unsubstituted alkoxy, substituted or unsubstituted heteroalkyl, substituted or unsubstituted cycloalkyl or substituted or unsubstituted heterocycloalkyl.

In one embodiment, R³ is a C₃-C₆cycloalkyl ring. In another embodiment, the C₃-C₆cycloalkyl ring is cyclopropyl. In another embodiment, the C₃-C₆cycloalkyl ring is cyclopentyl. In another embodiment, the C₃-C₆cycloalkyl is cyclohexyl. In yet another embodiment, R³ is a 5-6 membered heteroaryl ring wherein the 5-6 membered heteroaryl ring is attached to ring A via a carbon atom. In one embodiment, R³ is selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, and pyrazine. In one embodiment, R³ is pyridine. In another embodiment, R³ is thiazole. In a further embodiment, R³ is furan. In another embodiment, R³ is thiophene. In yet another embodiment, R³ is pyrazine.

In another embodiment, is a compound of Formula I, wherein R³ is substituted or unsubstituted heterocycloalkyl attached to ring A via a carbon atom. In one embodiment, R³ is substituted or unsubstituted piperidine. In another embodiment, R³ is substituted or unsubstituted pyrrolidine. In another embodiment, R³ is tetrahydrofuran. In yet another embodiment, R³ is tetrahydropyran.

In one embodiment is a compound of Formula I wherein

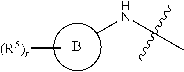

is selected from:

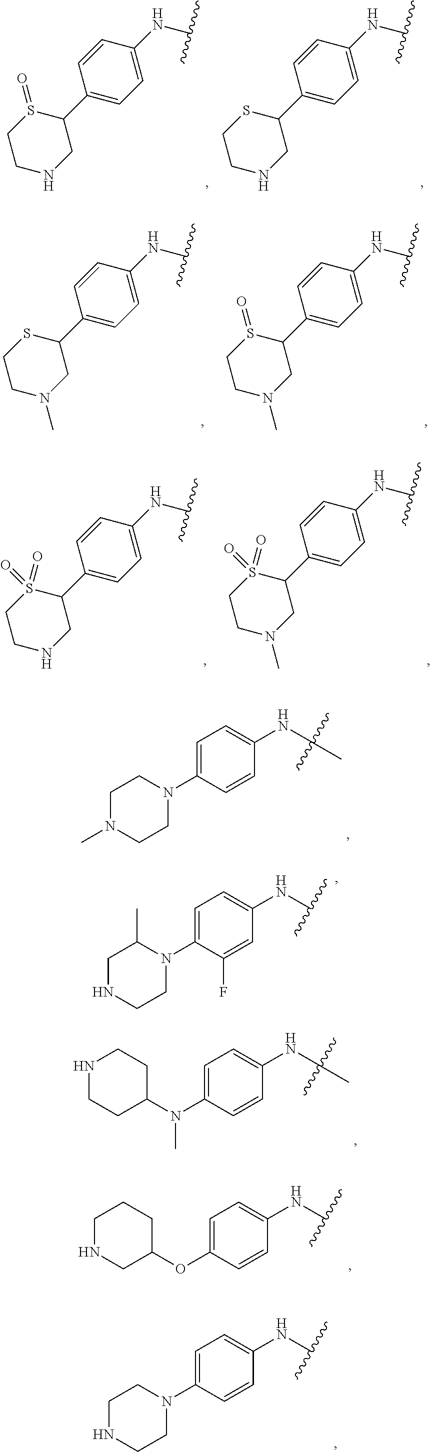

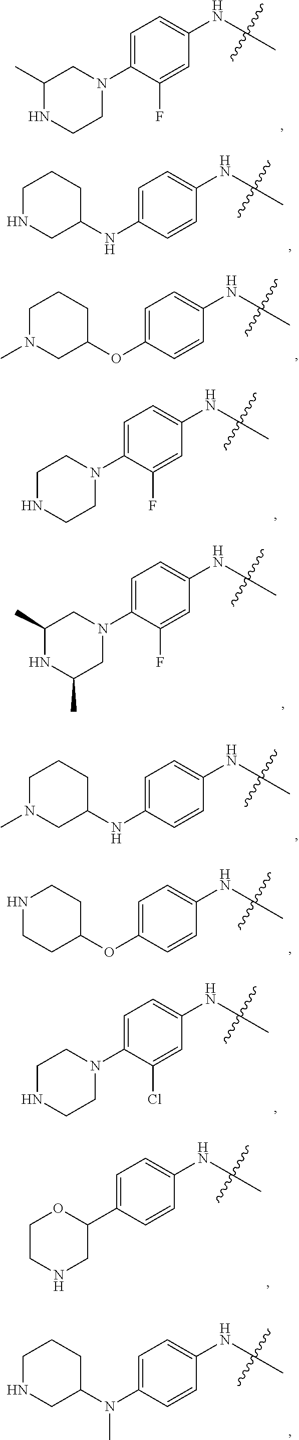

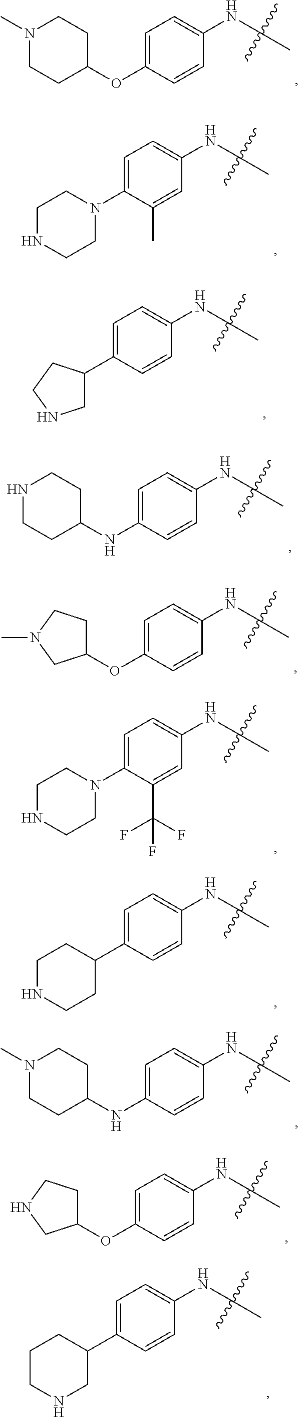

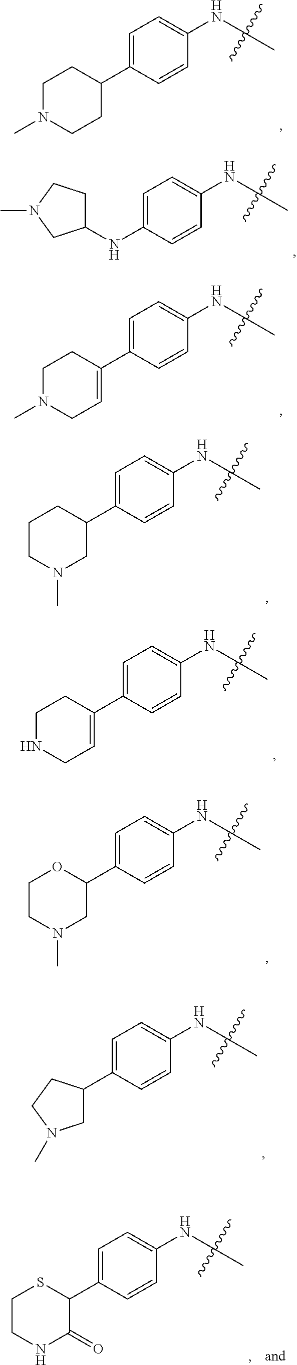

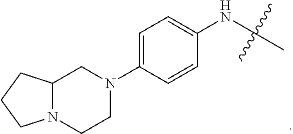 [SMILES: *Nc1ccc(N2CCN3CCCC3C2)cc1]

In yet another embodiment is a compound of Formula I wherein

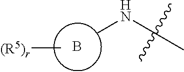

is selected from:

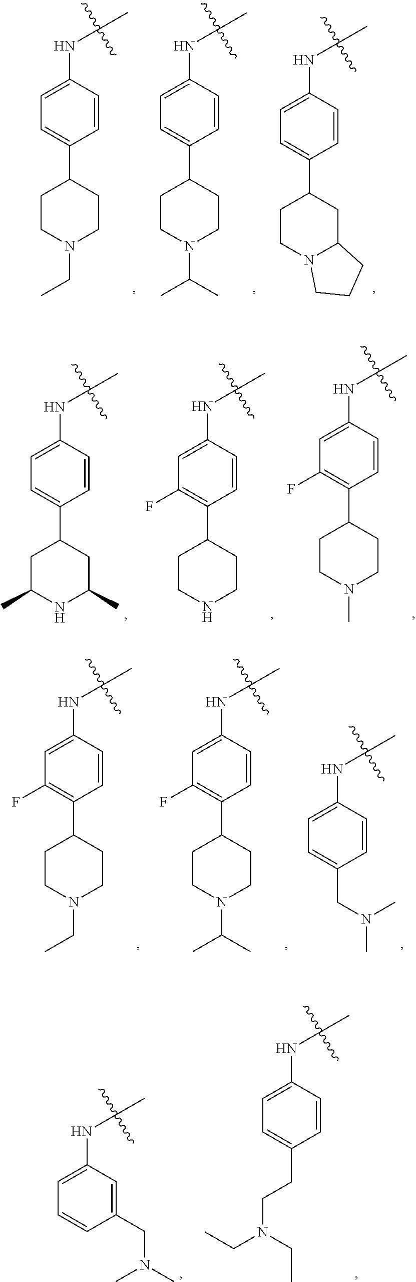

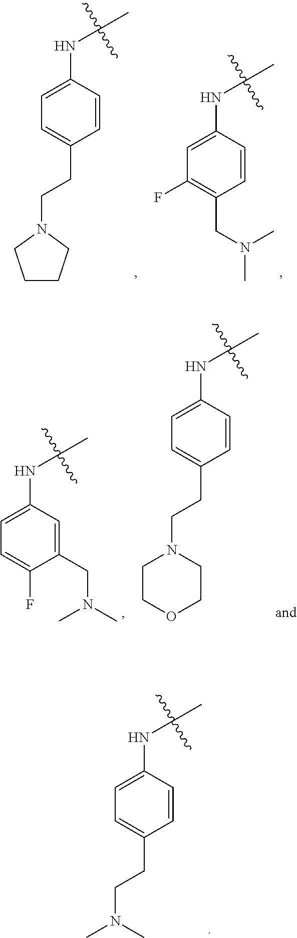

In one embodiment, is a compound of Formula I, wherein R⁵ is halogen, —CN, —OH, substituted or unsubstituted alkyl, —OR¹⁰, —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In one embodiment, R⁵ is selected from F, Cl, Br, or I. In another embodiment R⁵ is F.

In another embodiment, is a compound of Formula I, wherein at least one R⁵ is —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In one embodiment, is a compound of Formula I, wherein at least one R⁵ is)-N(R¹⁰)₂, or substituted or unsubstituted heterocycloalkyl. In yet another embodiment, is a compound of Formula I wherein at least one of R⁵ is a substituted or unsubstituted piperazine, substituted or unsubstituted piperidine, substituted or unsubstituted pyrrolidine or substituted or unsubstituted morpholine. In a further embodiment, is a compound of Formula I, wherein at least one R⁵ is —OR¹⁰. In one embodiment is a compound of Formula I, wherein at least one R⁵ is —OR¹⁰ and R¹⁰ is H. In another embodiment, R¹⁰ is alkyl selected from methyl, ethyl, n-propyl, iso-propyl, n-butyl, iso-butyl, and tert-butyl.

In another embodiment, is a compound of Formula I wherein R⁵ is a C₃-C₆cycloalkyl ring. In another embodiment, R⁵ is cyclopropyl. In another embodiment, R⁵ is cyclopentyl. In another embodiment, R⁵ is cyclohexyl.

In another embodiment, is a compound of Formula I wherein R⁵ is a substituted or unsubstituted heterocycloalkyl ring. In another embodiment, R⁵ is a substituted or unsubstituted piperazine. In another embodiment, R⁵ is an unsubstituted piperazine. In another embodiment, R⁵ is N-methylpiperazine. In another embodiment, R⁵ is a substituted or unsubstituted piperidine. In another embodiment, R⁵ is an unsubstituted piperidine.

In another embodiment is a compound of Formula I wherein r is 0. In another embodiment, r is 1. In a further embodiment, r is 2. In another embodiment is a compound of Formula I wherein r is 1; and R⁵ is substituted or unsubstituted piperazine.

In one embodiment is a compound of Formula I wherein ring B is substituted with —N(R¹⁰)₂, wherein R¹⁰ is each independently selected from H and a substituted or unsubstituted heterocycloalkyl. In another embodiment is a compound of Formula I wherein ring B is substituted with —NHR¹⁰ wherein R¹⁰ is a substituted or unsubstituted piperazine, substituted or unsubstituted piperidine, substituted or unsubstituted pyrrolidine or substituted or unsubstituted morpholine. In a further embodiment is a compound of Formula I wherein ring B is substituted with —N(CH₃)R¹⁰ wherein R¹⁰ is a substituted or unsubstituted piperazine, substituted or unsubstituted piperidine, substituted or unsubstituted pyrrolidine or substituted or unsubstituted morpholine.

Also presented herein is a compound of Formula I wherein ring B is substituted with —OR¹⁰ wherein R¹⁰ is a substituted or unsubstituted heterocycloalkyl. In another embodiment is a compound of Formula I wherein ring B is substituted with —OR¹⁰ wherein R¹⁰ is a substituted or unsubstituted piperazine, substituted or unsubstituted piperidine, substituted or unsubstituted pyrrolidine or substituted or unsubstituted morpholine. In yet another embodiment is a compound of Formula I wherein ring B is substituted with at least one CF₃.

In yet another embodiment, ring B is substituted with at least two R⁵. In another embodiment, ring B is substituted with halogen and a substituted or unsubstituted heterocycloalkyl. In another embodiment, ring B is substituted with at least one F, Cl, Br, or I and a substituted or unsubstituted piperazine, substituted or unsubstituted piperidine, substituted or unsubstituted pyrrolidine, or substituted or unsubstituted morpholine.

In yet a further embodiment, is a compound of Formula I, wherein R⁴ is independently halogen, —CN, —OH, —OCF₃, —OCHF₂, —OCF₂H, —CF₃, —SR⁸, —S(═O)R⁹, —S(═O)₂R⁹, substituted or unsubstituted alkyl, or substituted or unsubstituted alkoxy. In one embodiment, R⁴ is halogen. In another embodiment, R⁴ is selected from F, Cl, Br, and I. In a further embodiment, R⁴ is F.

In a further embodiment is a compound of Formula I wherein

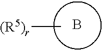

is:

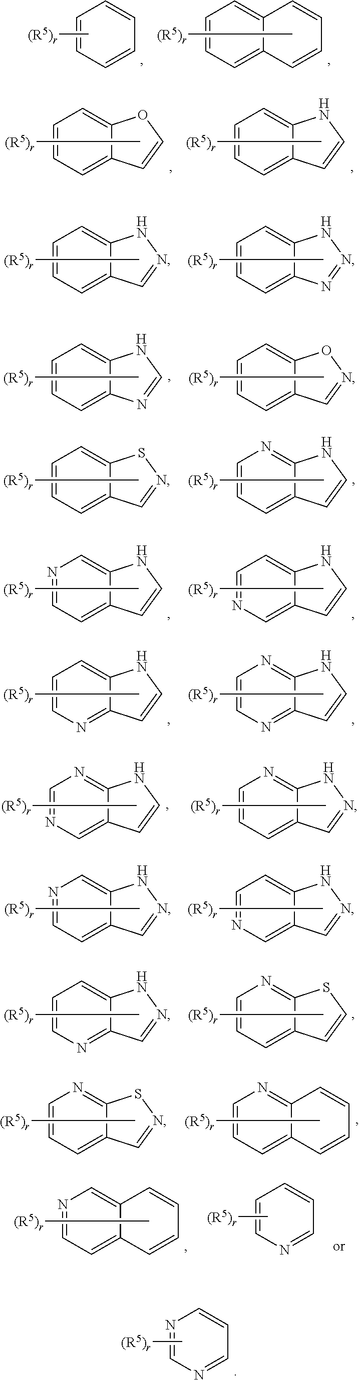

In another embodiment is a compound of Formula I where R⁵ is halogen, —CN, —OH, a substituted or unsubstituted alkyl, —OR¹⁰, —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or a substituted or unsubstituted heterocycloalkyl.

In one embodiment is a compound of Formula I wherein at least one R⁵ is —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or a substituted or unsubstituted heterocycloalkyl.

In one embodiment is a compound of Formula I wherein at least one R⁵ is —N(R¹⁰)₂, or a substituted or unsubstituted heterocycloalkyl. In a further embodiment is a compound of Formula I wherein at least one of R⁵ is a substituted or unsubstituted piperazine, a substituted or unsubstituted piperidine, a substituted or unsubstituted pyrrolidine, or a substituted or unsubstituted morpholine. In one embodiment is a compound of Formula I wherein at least one R⁵ is —OR¹⁰. In another embodiment is a compound of Formula I wherein R⁴ is independently halogen, —CN, —OH, —OCF₃, —OCF₃, —OCF₂H, —CF₃, —SR⁸, a substituted or unsubstituted alkyl, or a substituted or unsubstituted alkoxy.

In one embodiment, is a compound of Formula I, wherein s is zero.

In some embodiments are PAK inhibitors suitable for treating cancer having the structure of Formula Ia:

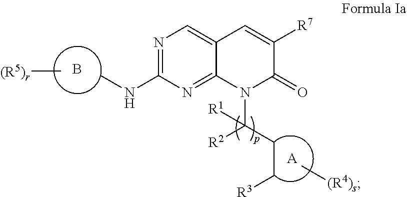

wherein:
R¹ and R² are each independently H or substituted or unsubstituted alkyl; or R¹ and R² together with the carbon to which they are attached form a substituted or unsubstituted C₃-C₆ cycloalkyl ring;
p is 1, 2 or 3; ring A is heteroaryl comprising 1-4 heteroatoms selected from O, S and N;
R³ is a substituted or unsubstituted cycloalkyl, substituted or unsubstituted heteroaryl attached to ring A via a carbon atom, or substituted or unsubstituted heterocycloalkyl attached to ring A via a carbon atom;
each R⁴ is independently halogen, —CN, —NO₂, —OH, —OCF₃, —OCHF₂, —OCF₂H, —CF₃, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
R⁸ is H or substituted or unsubstituted alkyl, substituted or unsubstituted cycloalkyl, substituted or unsubstituted heterocycloalkyl, substituted or unsubstituted aryl or substituted or unsubstituted heteroaryl;
R⁹ is substituted or unsubstituted alkyl, substituted or unsubstituted cycloalkyl, substituted or unsubstituted heterocycloalkyl, substituted or unsubstituted aryl or substituted or unsubstituted heteroaryl;
each R¹⁰ is independently H, substituted or unsubstituted alkyl, substituted or unsubstituted cycloalkyl, substituted or unsubstituted heterocycloalkyl substituted or unsubstituted aryl or substituted or unsubstituted heteroaryl, or two R¹⁰ together with the atoms to which they are attached form a substituted or unsubstituted heterocycle;
s is 0-4;
ring B is aryl or heteroaryl;
each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8; and
R⁷ is H, halogen, —CN, substituted or unsubstituted alkyl, —C(═O)N(R¹⁰)₂, —CO₂R¹⁰, —OR¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In one embodiment, is a compound of Formula I or Ia, wherein p is 1; and R¹ and R² are each H. In another embodiment, is a compound of Formula I or Ia, wherein R⁷ is H. In yet another embodiment, is a compound of Formula I or Ia wherein R⁷ is —CN. In a further embodiment, is a compound of Formula I or Ia wherein R⁷ is substituted or unsubstituted cyclopropyl, substituted or unsubstituted cyclobutyl, substituted or unsubstituted cyclopentyl or substituted or unsubstituted cyclohexyl. In another embodiment, R⁷ is —OR¹⁰.

In yet a further embodiment, is a compound of Formula I or Ia wherein R⁷ is substituted or unsubstituted morpholino, substituted or unsubstituted piperazinyl, substituted or unsubstituted piperidinyl.

In one embodiment, is a compound of Formula I or Ia wherein R⁷ is substituted or unsubstituted acyl.

In another embodiment, is a compound of Formula I or Ia, wherein R⁷ is substituted or unsubstituted aryl or substituted or unsubstituted heteroaryl.

In yet another embodiment, is a compound of Formula I or Ia wherein substituted or unsubstituted aryl or substituted or unsubstituted heteroaryl has the structure:

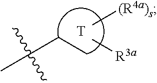

wherein:
R^{4a} is H or R^{3a};
R^{3a} is halogen, —CN, —NO₂, —OH, —OCF₃, —OCF₂H, —CF₃, —SR⁸, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR⁹, substituted or unsubstituted alkyl, substituted or unsubstituted heteroalkyl, substituted or unsubstituted cycloalkyl or substituted or unsubstituted heterocycloalkyl;
R⁸ is H or substituted or unsubstituted alkyl, substituted or unsubstituted cycloalkyl, substituted or unsubstituted heterocycloalkyl, substituted or unsubstituted aryl or substituted or unsubstituted heteroaryl;
R⁹ is substituted or unsubstituted alkyl, substituted or unsubstituted cycloalkyl, substituted or unsubstituted aryl or substituted or unsubstituted heteroaryl, substituted or unsubstituted heterocycloalkyl;
each R¹⁰ is independently H, substituted or unsubstituted alkyl, substituted or unsubstituted cycloalkyl, substituted or unsubstituted aryl or substituted or unsubstituted heteroaryl, substituted or unsubstituted heterocycloalkyl, or two R¹⁰ together with the atoms to which they are attached form a substituted or unsubstituted heterocycle; and
   s is 0-4.

In a further embodiment, is a compound of Formula I or Ia, wherein R³ is cyclopropyl, cyclobutyl, morpholino, piperidinyl, tetrahydropyran, tetrahydrofuranyl, pyrrolidinyl, or piperazinyl.

In yet a further embodiment, is a compound of Formula I or Ia, wherein R³ is heteroaryl selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine and pyrazine.

In yet another embodiment is a compound of Formula I or Ia wherein R⁷ is selected from:

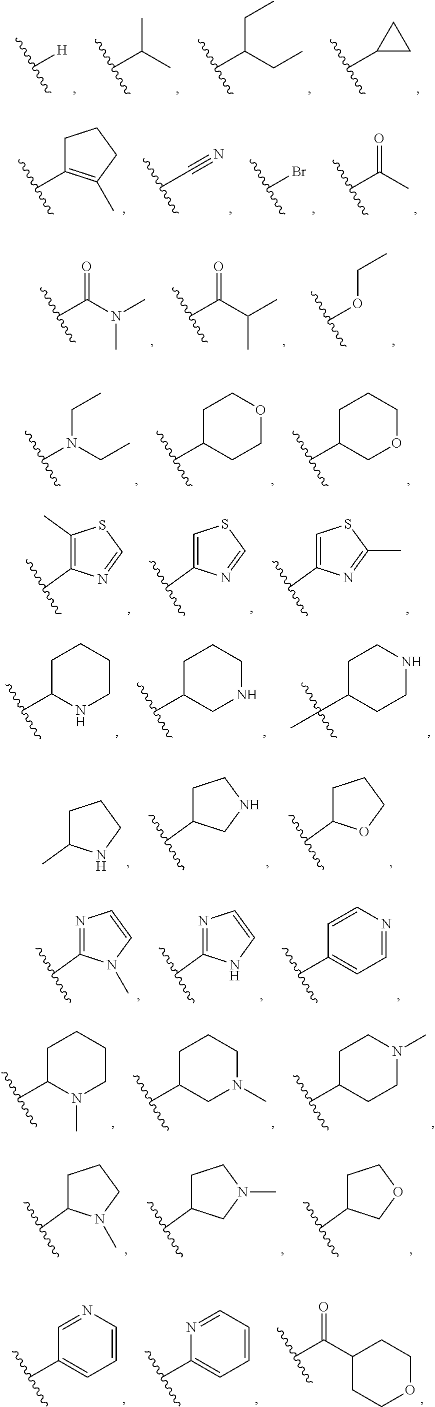

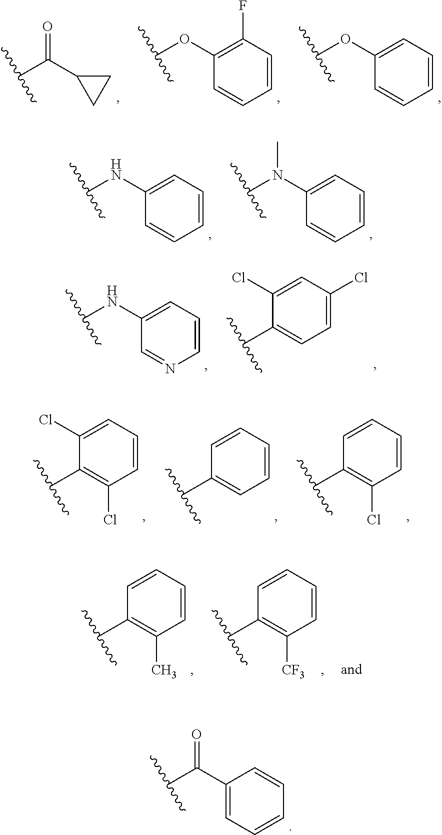

In a further embodiment is a compound having the structure:

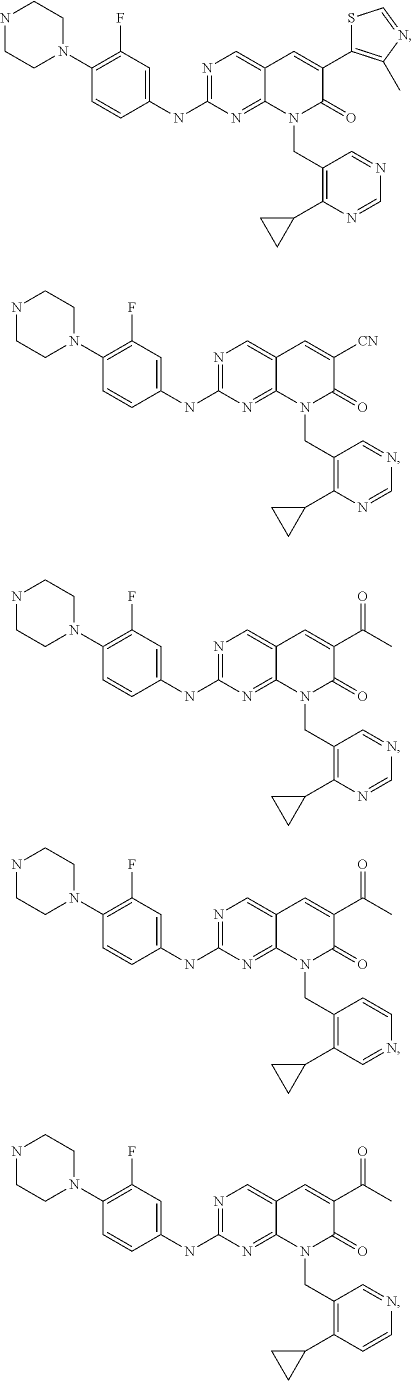 [SMILES: Cc1ncsc1-c1cc2cnc(Nc3ccc(N4CCNCC4)c(F)c3)nc2n(Cc2cncnc2C2CC2)c1=O ; N#Cc1cc2cnc(Nc3ccc(N4CCNCC4)c(F)c3)nc2n(Cc2cncnc2C2CC2)c1=O ; CC(=O)c1cc2cnc(Nc3ccc(N4CCNCC4)c(F)c3)nc2n(Cc2cncnc2C2CC2)c1=O ; CC(=O)c1cc2cnc(Nc3ccc(N4CCNCC4)c(F)c3)nc2n(Cc2ccncc2C2CC2)c1=O ; CC(=O)c1cc2cnc(Nc3ccc(N4CCNCC4)c(F)c3)nc2n(Cc2cnccc2C2CC2)c1=O]

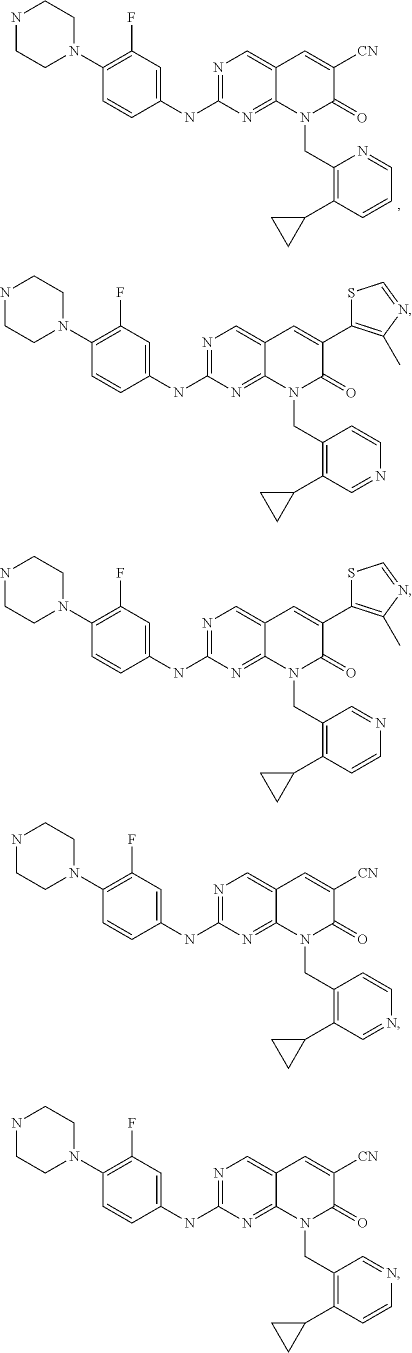 [SMILES: N#Cc1cc2cnc(Nc3ccc(N4CCNCC4)c(F)c3)nc2n(Cc2ncccc2C2CC2)c1=O ; Cc1ncsc1-c1cc2cnc(Nc3ccc(N4CCNCC4)c(F)c3)nc2n(Cc2ccncc2C2CC2)c1=O ; Cc1ncsc1-c1cc2cnc(Nc3ccc(N4CCNCC4)c(F)c3)nc2n(Cc2cnccc2C2CC2)c1=O ; N#Cc1cc2cnc(Nc3ccc(N4CCNCC4)c(F)c3)nc2n(Cc2ccncc2C2CC2)c1=O ; N#Cc1cc2cnc(Nc3ccc(N4CCNCC4)c(F)c3)nc2n(Cc2cnccc2C2CC2)c1=O]

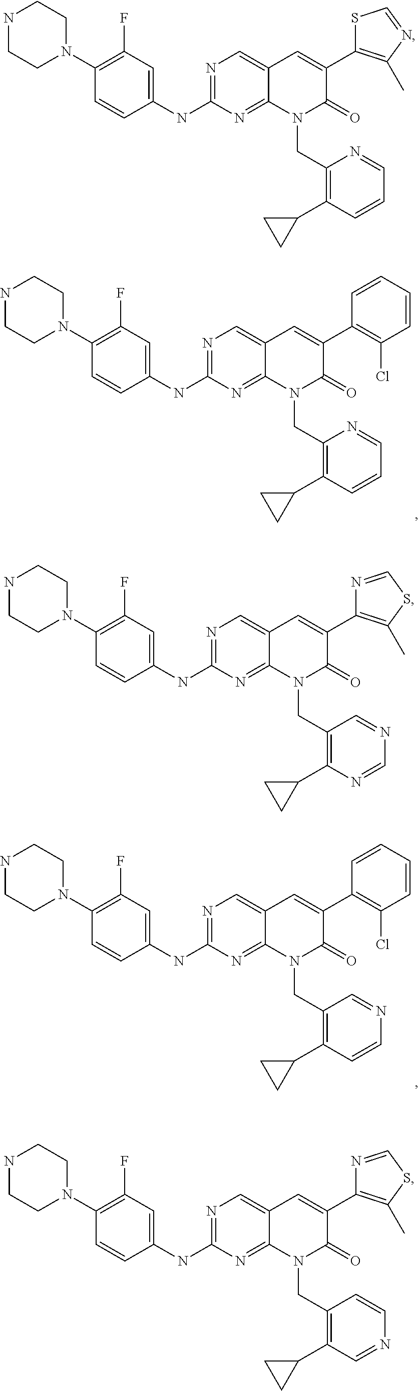 [SMILES: Cc1ncsc1-c1cc2cnc(Nc3ccc(N4CCNCC4)c(F)c3)nc2n(Cc2ncccc2C2CC2)c1=O ; O=c1c(-c2ccccc2Cl)cc2cnc(Nc3ccc(N4CCNCC4)c(F)c3)nc2n1Cc1ncccc1C1CC1 ; Cc1scnc1-c1cc2cnc(Nc3ccc(N4CCNCC4)c(F)c3)nc2n(Cc2cncnc2C2CC2)c1=O ; O=c1c(-c2ccccc2Cl)cc2cnc(Nc3ccc(N4CCNCC4)c(F)c3)nc2n1Cc1cnccc1C1CC1 ; Cc1scnc1-c1cc2cnc(Nc3ccc(N4CCNCC4)c(F)c3)nc2n(Cc2ccncc2C2CC2)c1=O]

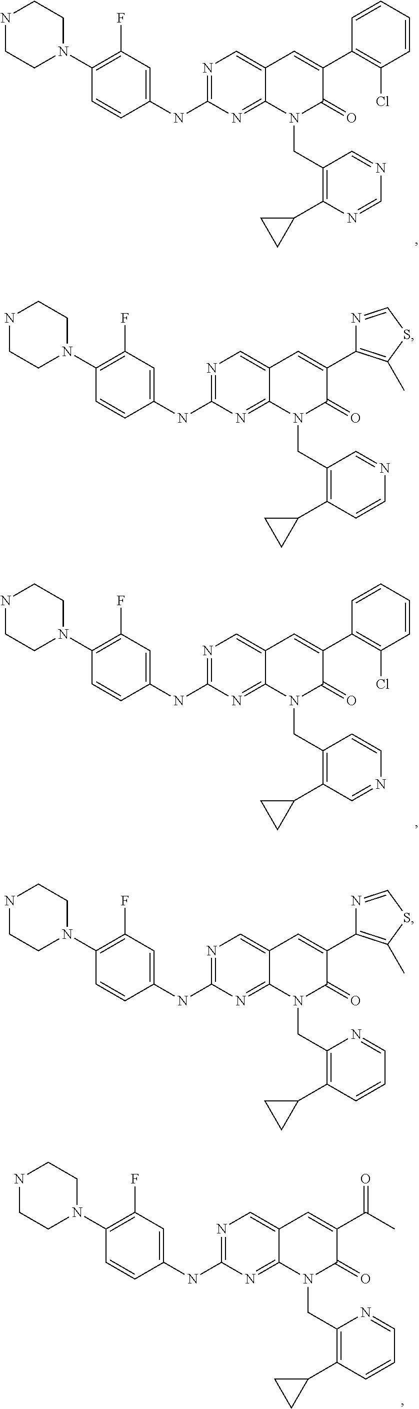 [SMILES: O=c1c(-c2ccccc2Cl)cc2cnc(Nc3ccc(N4CCNCC4)c(F)c3)nc2n1Cc1cncnc1C1CC1 ; Cc1scnc1-c1cc2cnc(Nc3ccc(N4CCNCC4)c(F)c3)nc2n(Cc2cnccc2C2CC2)c1=O ; O=c1c(-c2ccccc2Cl)cc2cnc(Nc3ccc(N4CCNCC4)c(F)c3)nc2n1Cc1ccncc1C1CC1 ; Cc1scnc1-c1cc2cnc(Nc3ccc(N4CCNCC4)c(F)c3)nc2n(Cc2ncccc2C2CC2)c1=O ; CC(=O)c1cc2cnc(Nc3ccc(N4CCNCC4)c(F)c3)nc2n(Cc2ncccc2C2CC2)c1=O]

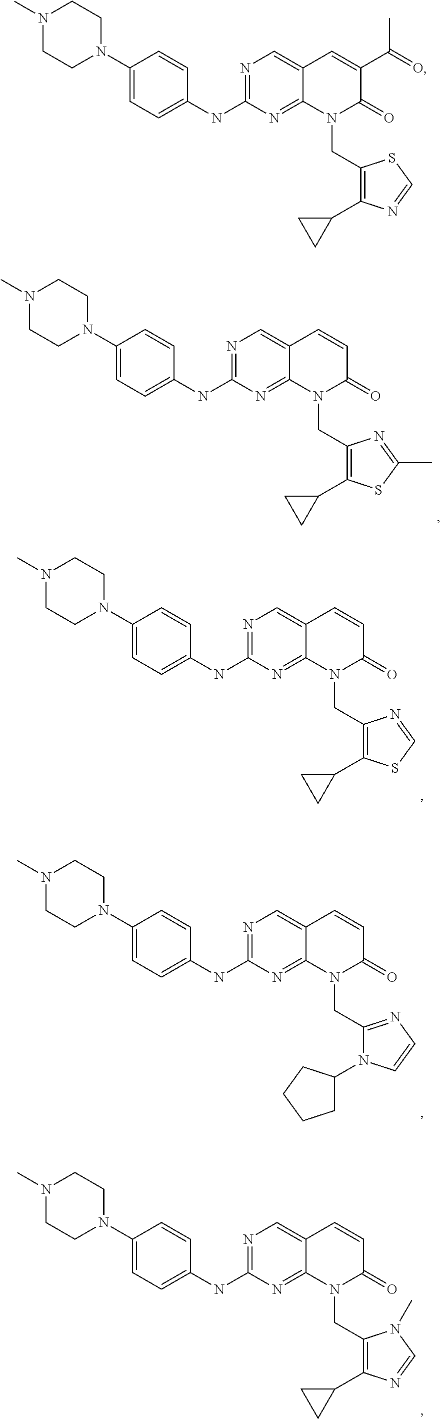

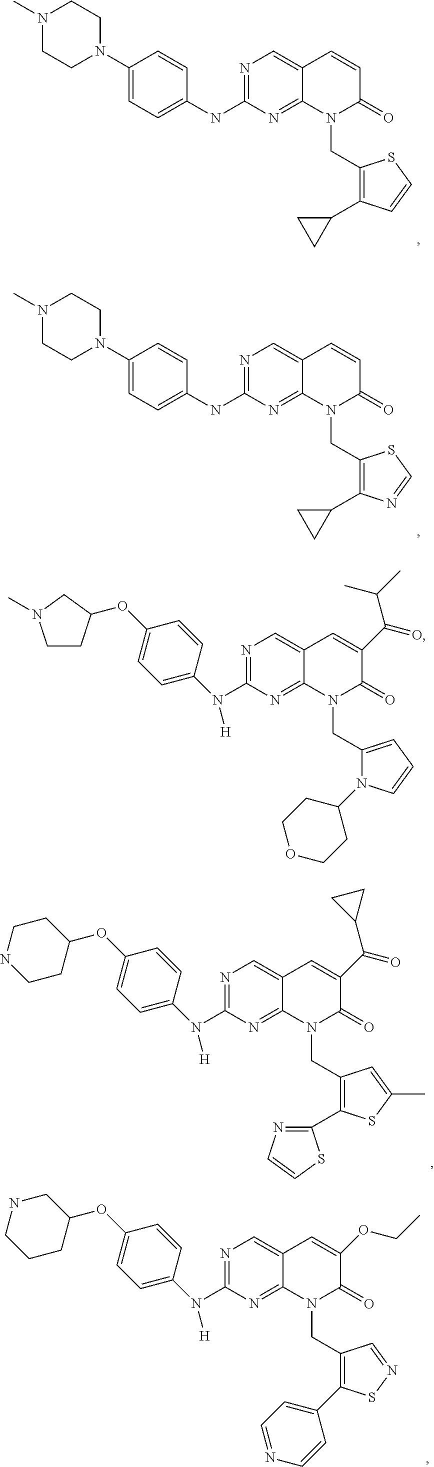

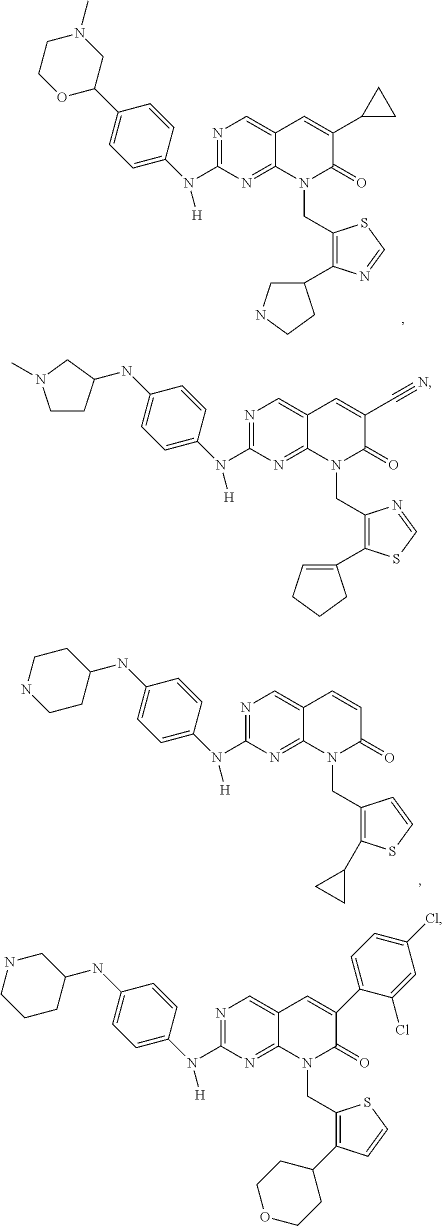

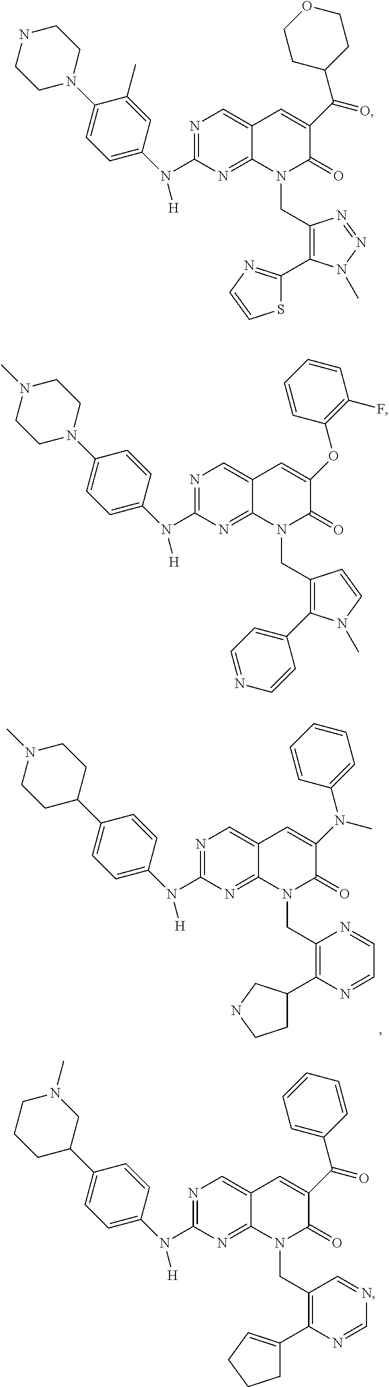 [SMILES: Cc1cc(Nc2ncc3cc(C(=O)C4CCOCC4)c(=O)n(Cc4nnn(C)c4-c4nccs4)c3n2)ccc1N1CCNCC1 ; CN1CCN(c2ccc(Nc3ncc4cc(Oc5ccccc5F)c(=O)n(Cc5ccn(C)c5-c5ccncc5)c4n3)cc2)CC1 ; CN1CCC(c2ccc(Nc3ncc4cc(N(C)c5ccccc5)c(=O)n(Cc5nccnc5C5CCNC5)c4n3)cc2)CC1 ; CN1CCCC(c2ccc(Nc3ncc4cc(C(=O)c5ccccc5)c(=O)n(Cc5cncnc5C5=CCCC5)c4n3)cc2)C1]

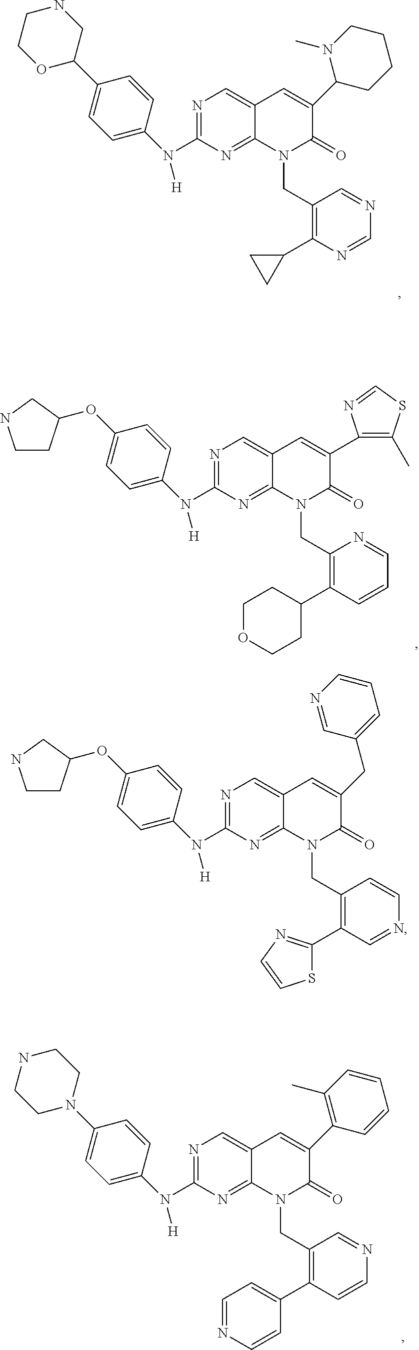

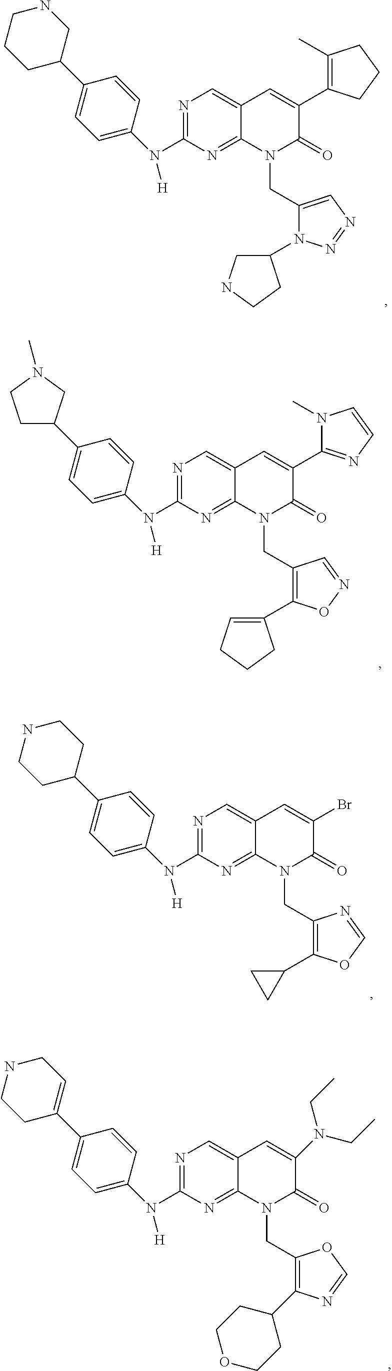

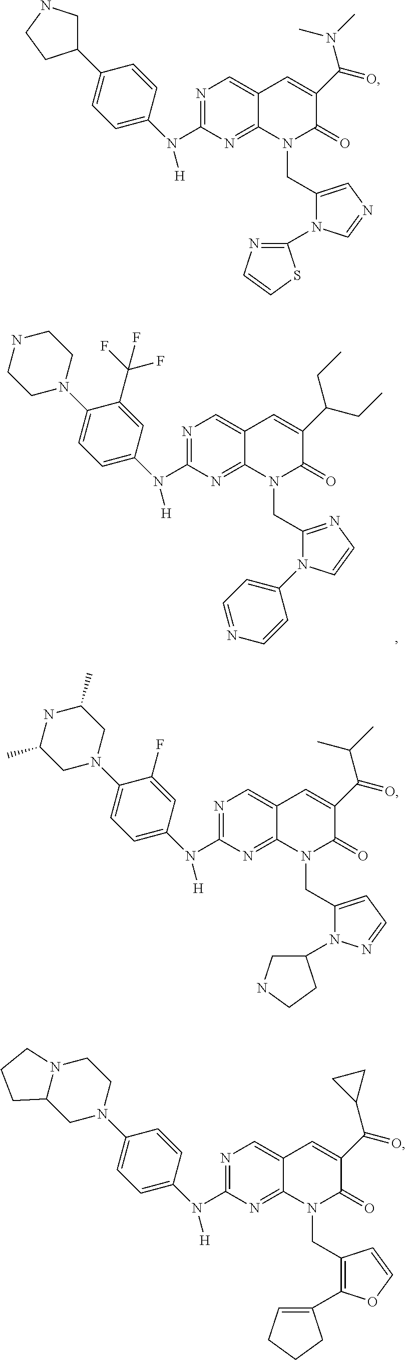

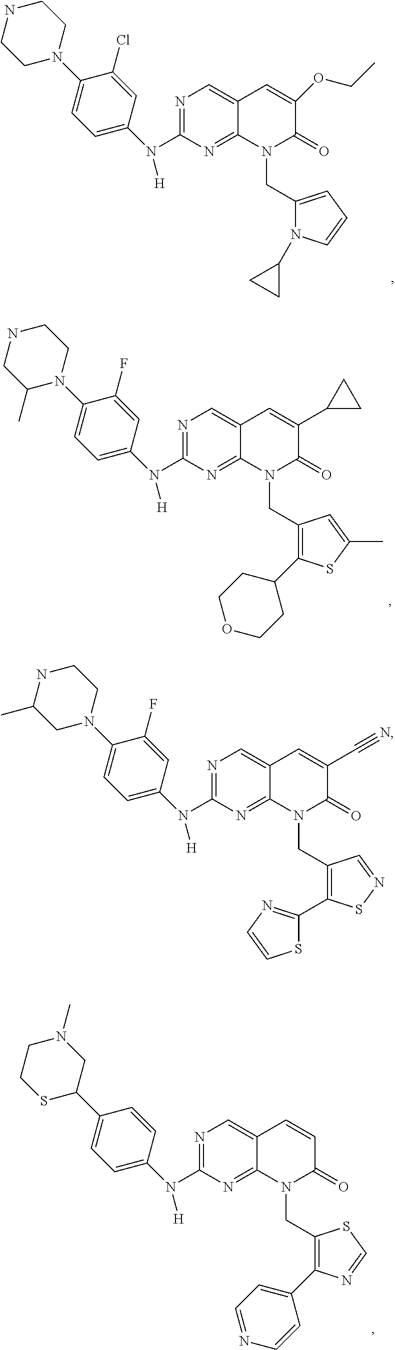

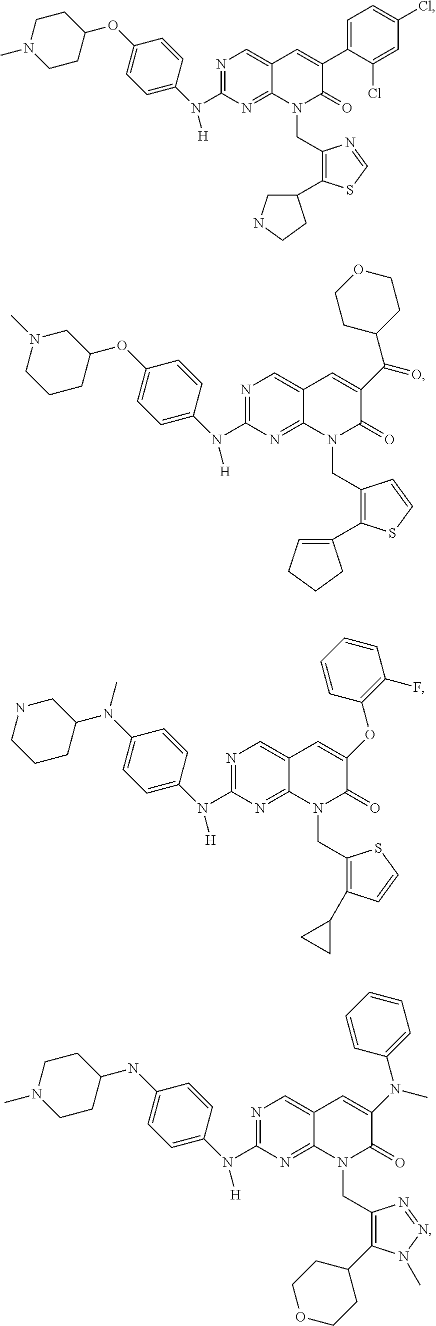

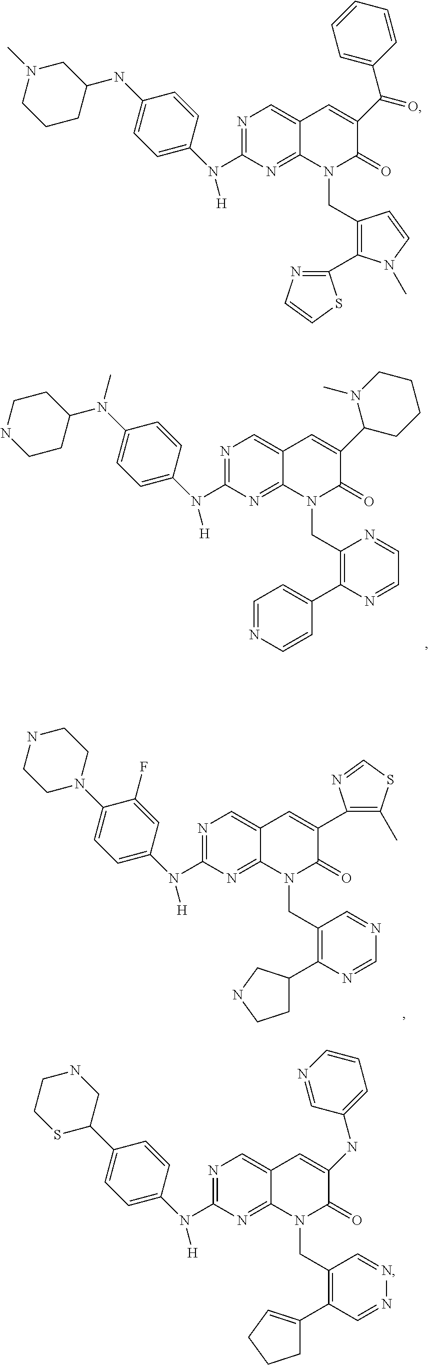 [SMILES: CN1CCCC(Nc2ccc(Nc3ncc4cc(C(=O)c5ccccc5)c(=O)n(Cc5ccn(C)c5-c5nccs5)c4n3)cc2)C1 ; CN1CCCCC1c1cc2cnc(Nc3ccc(N(C)C4CCNCC4)cc3)nc2n(Cc2nccnc2-c2ccncc2)c1=O ; Cc1scnc1-c1cc2cnc(Nc3ccc(N4CCNCC4)c(F)c3)nc2n(Cc2cncnc2C2CCNC2)c1=O ; O=c1c(Nc2cccnc2)cc2cnc(Nc3ccc(C4CNCCS4)cc3)nc2n1Cc1cnncc1C1=CCCC1]

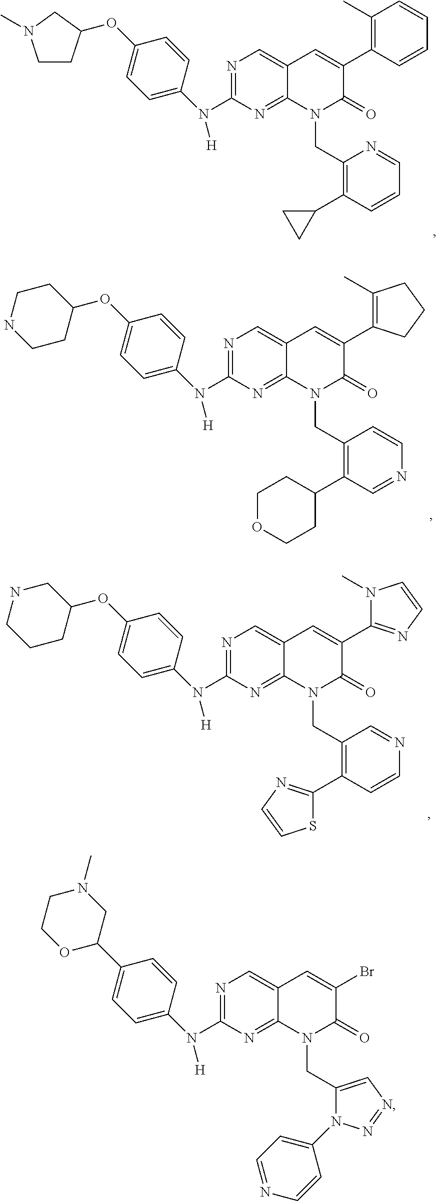 [SMILES: Cc1ccccc1-c1cc2cnc(Nc3ccc(OC4CCN(C)C4)cc3)nc2n(Cc2ncccc2C2CC2)c1=O ; CC1=C(c2cc3cnc(Nc4ccc(OC5CCNCC5)cc4)nc3n(Cc3ccncc3C3CCOCC3)c2=O)CCC1 ; Cn1ccnc1-c1cc2cnc(Nc3ccc(OC4CCCNC4)cc3)nc2n(Cc2cnccc2-c2nccs2)c1=O ; CN1CCOC(c2ccc(Nc3ncc4cc(Br)c(=O)n(Cc5cnnn5-c5ccncc5)c4n3)cc2)C1]

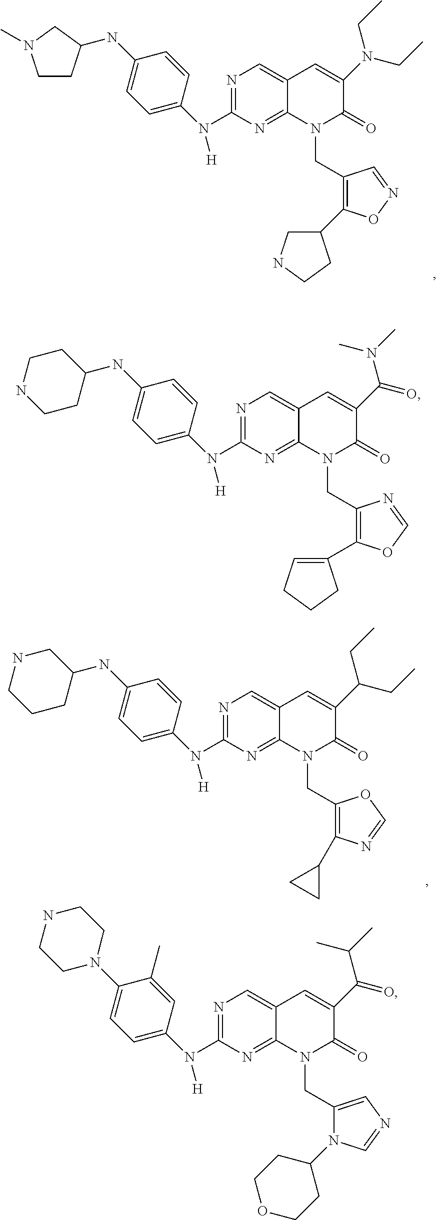

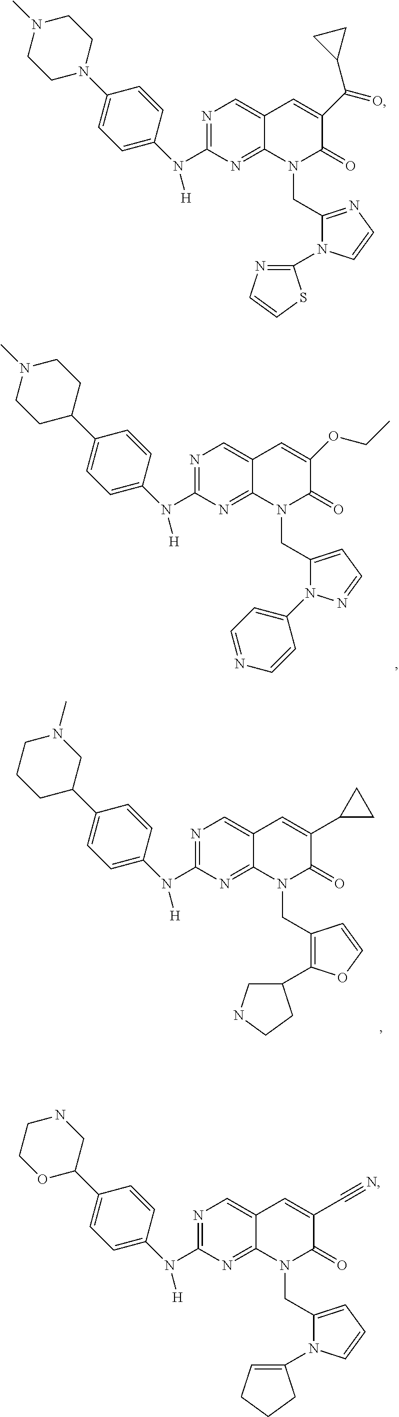 [SMILES: CN1CCN(c2ccc(Nc3ncc4cc(C(=O)C5CC5)c(=O)n(Cc5nccn5-c5nccs5)c4n3)cc2)CC1 ; CCOc1cc2cnc(Nc3ccc(C4CCN(C)CC4)cc3)nc2n(Cc2ccnn2-c2ccncc2)c1=O ; CN1CCCC(c2ccc(Nc3ncc4cc(C5CC5)c(=O)n(Cc5ccoc5C5CCNC5)c4n3)cc2)C1 ; N#Cc1cc2cnc(Nc3ccc(C4CNCCO4)cc3)nc2n(Cc2cccn2C2=CCCC2)c1=O]

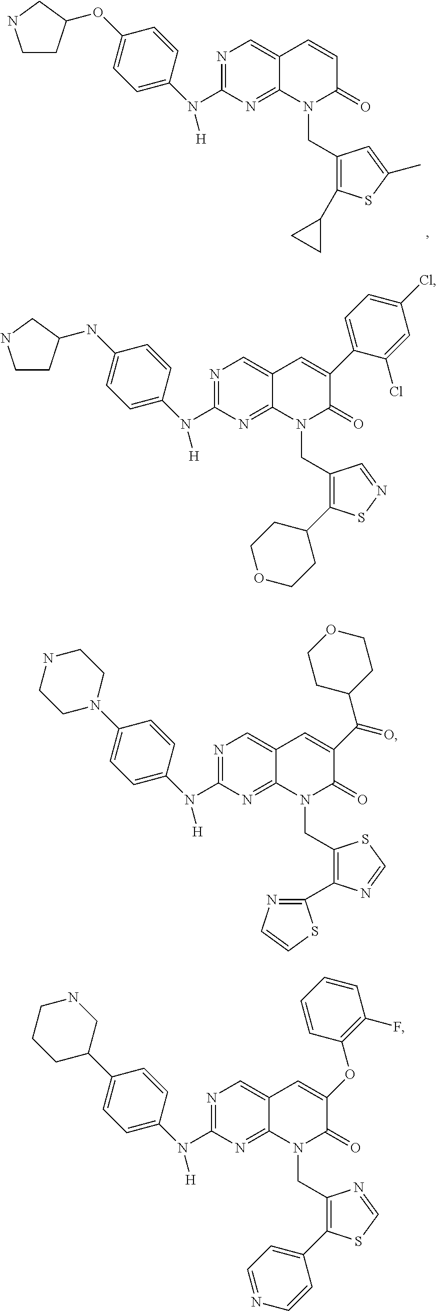

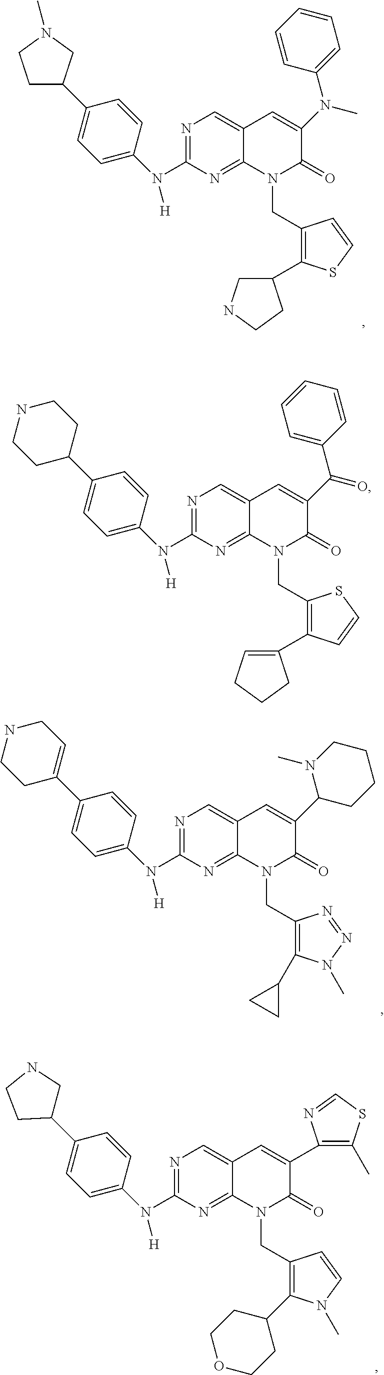 [SMILES: CN1CCC(c2ccc(Nc3ncc4cc(N(C)c5ccccc5)c(=O)n(Cc5ccsc5C5CCNC5)c4n3)cc2)C1 ; O=C(c1ccccc1)c1cc2cnc(Nc3ccc(C4CCNCC4)cc3)nc2n(Cc2sccc2C2=CCCC2)c1=O ; CN1CCCCC1c1cc2cnc(Nc3ccc(C4=CCNCC4)cc3)nc2n(Cc2nnn(C)c2C2CC2)c1=O ; Cc1scnc1-c1cc2cnc(Nc3ccc(C4CCNC4)cc3)nc2n(Cc2ccn(C)c2C2CCOCC2)c1=O]

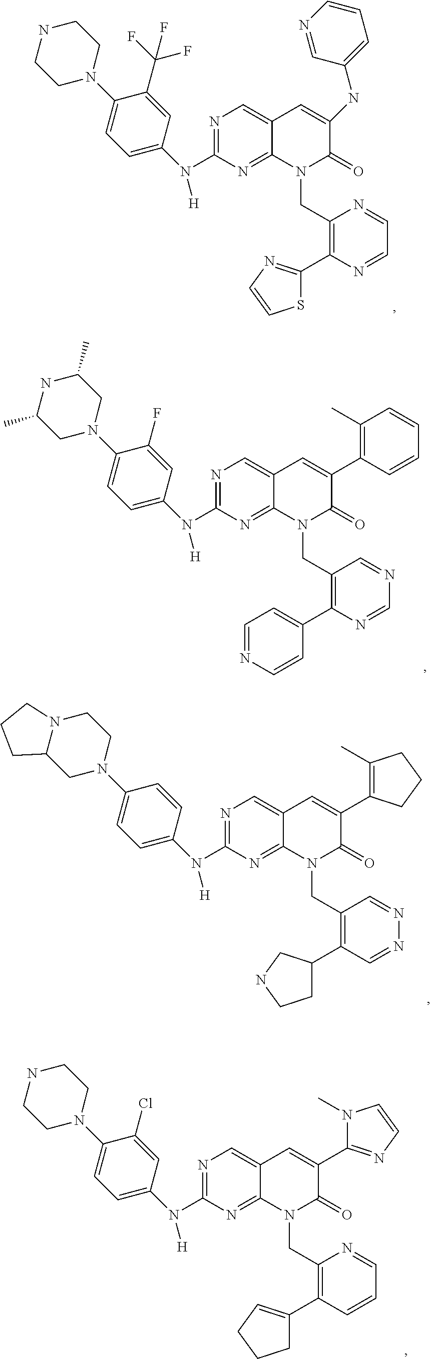 [SMILES: O=c1c(Nc2cccnc2)cc2cnc(Nc3ccc(N4CCNCC4)c(C(F)(F)F)c3)nc2n1Cc1nccnc1-c1nccs1 ; Cc1ccccc1-c1cc2cnc(Nc3ccc(N4C[C@@H](C)N[C@@H](C)C4)c(F)c3)nc2n(Cc2cncnc2-c2ccncc2)c1=O ; CC1=C(c2cc3cnc(Nc4ccc(N5CCN6CCCC6C5)cc4)nc3n(Cc3cnncc3C3CCNC3)c2=O)CCC1 ; Cn1ccnc1-c1cc2cnc(Nc3ccc(N4CCNCC4)c(Cl)c3)nc2n(Cc2ncccc2C2=CCCC2)c1=O]

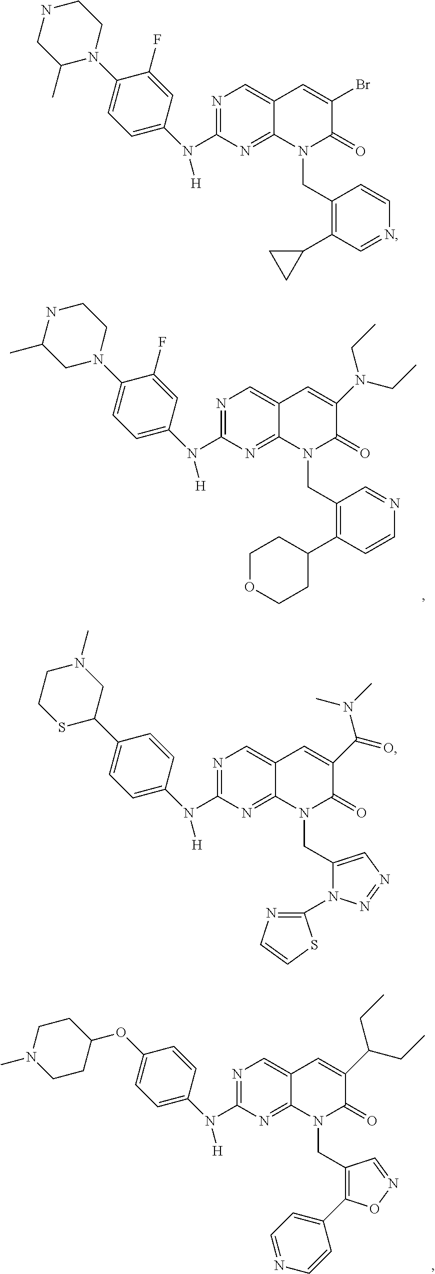

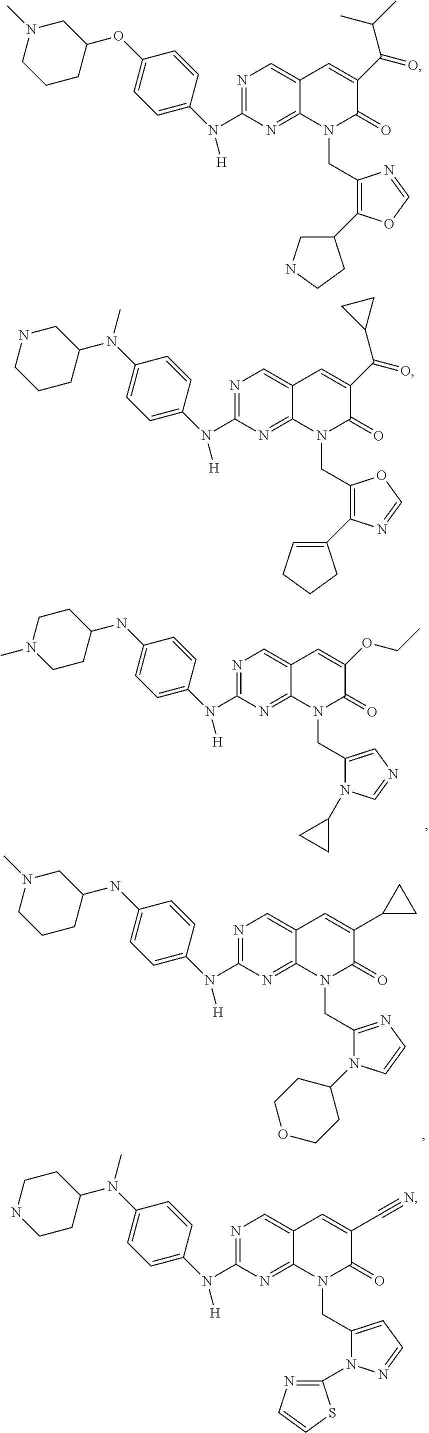

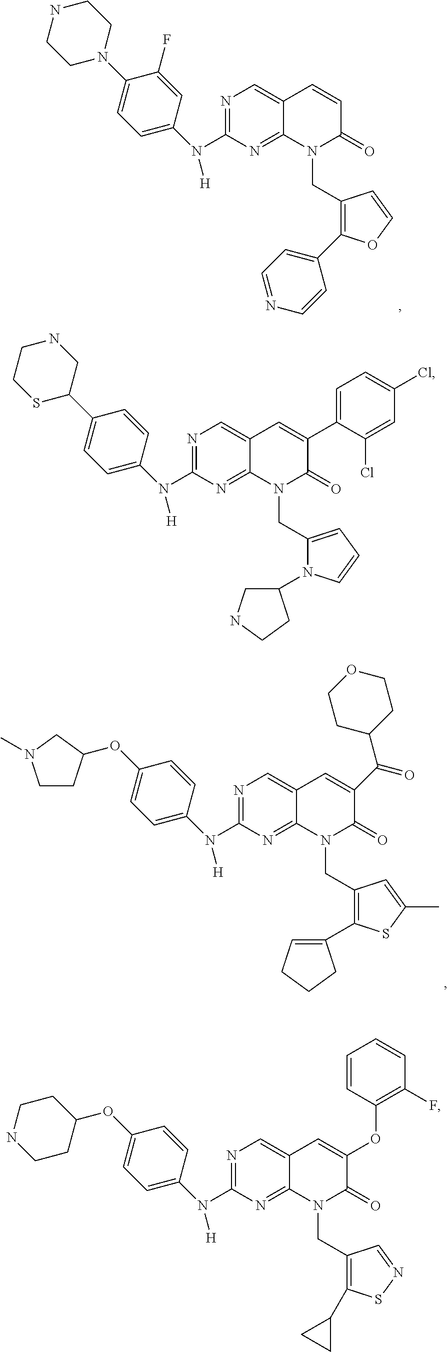

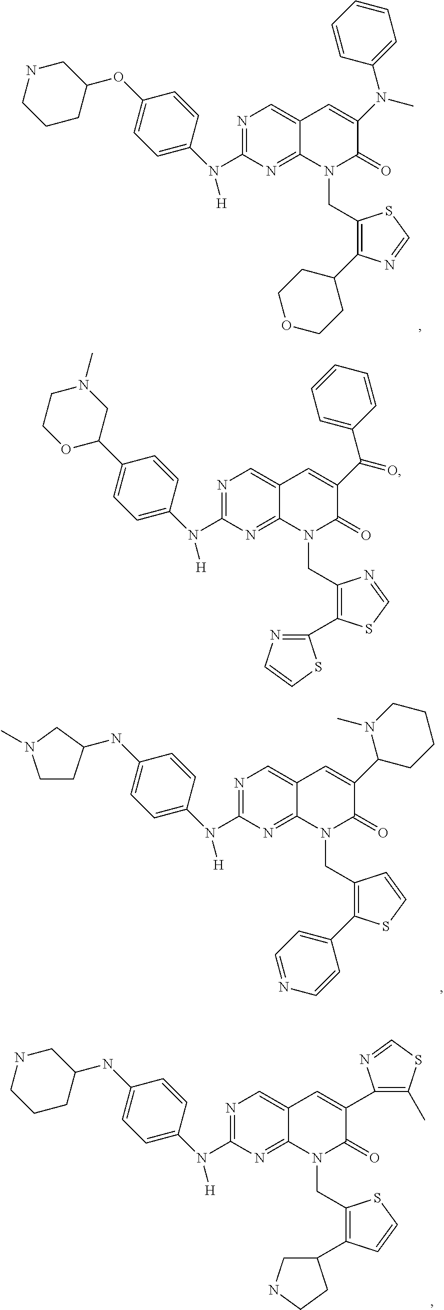

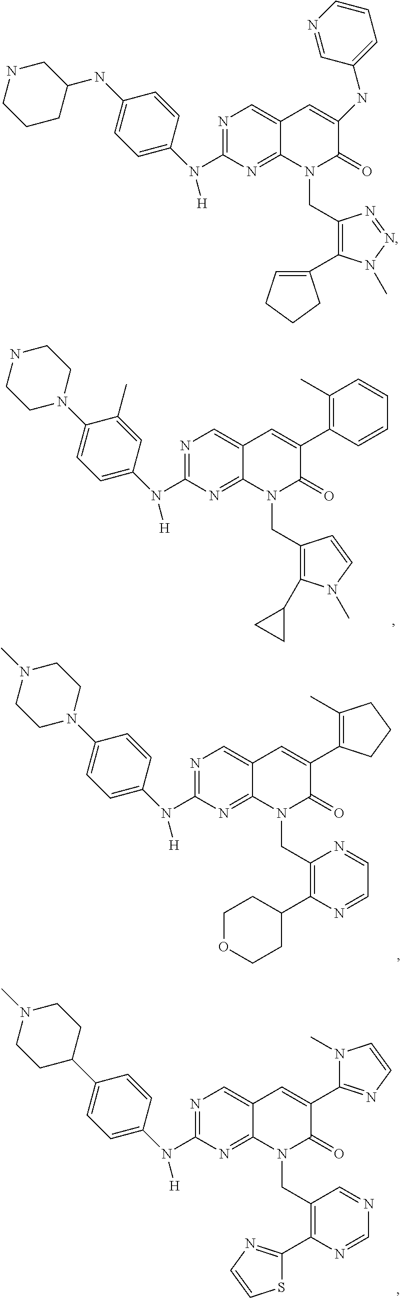 [SMILES: Cn1nnc(Cn2c(=O)c(Nc3cccnc3)cc3cnc(Nc4ccc(NC5CCCNC5)cc4)nc32)c1C1=CCCC1 ; Cc1ccccc1-c1cc2cnc(Nc3ccc(N4CCNCC4)c(C)c3)nc2n(Cc2ccn(C)c2C2CC2)c1=O ; CC1=C(c2cc3cnc(Nc4ccc(N5CCN(C)CC5)cc4)nc3n(Cc3nccnc3C3CCOCC3)c2=O)CCC1 ; CN1CCC(c2ccc(Nc3ncc4cc(-c5nccn5C)c(=O)n(Cc5cncnc5-c5nccs5)c4n3)cc2)CC1]

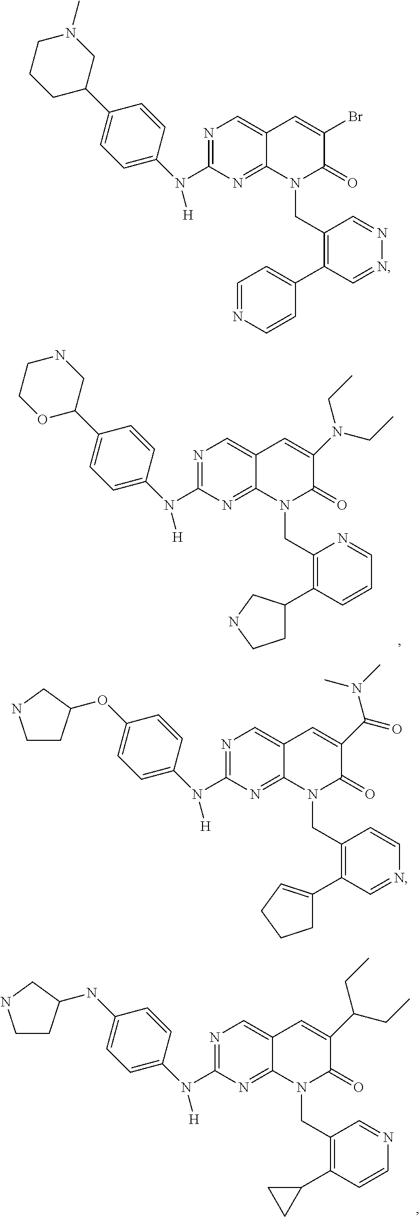 [SMILES: CN1CCCC(c2ccc(Nc3ncc4cc(Br)c(=O)n(Cc5cnncc5-c5ccncc5)c4n3)cc2)C1 ; CCN(CC)c1cc2cnc(Nc3ccc(C4CNCCO4)cc3)nc2n(Cc2ncccc2C2CCNC2)c1=O ; CN(C)C(=O)c1cc2cnc(Nc3ccc(OC4CCNC4)cc3)nc2n(Cc2ccncc2C2=CCCC2)c1=O ; CCC(CC)c1cc2cnc(Nc3ccc(NC4CCNC4)cc3)nc2n(Cc2cnccc2C2CC2)c1=O]

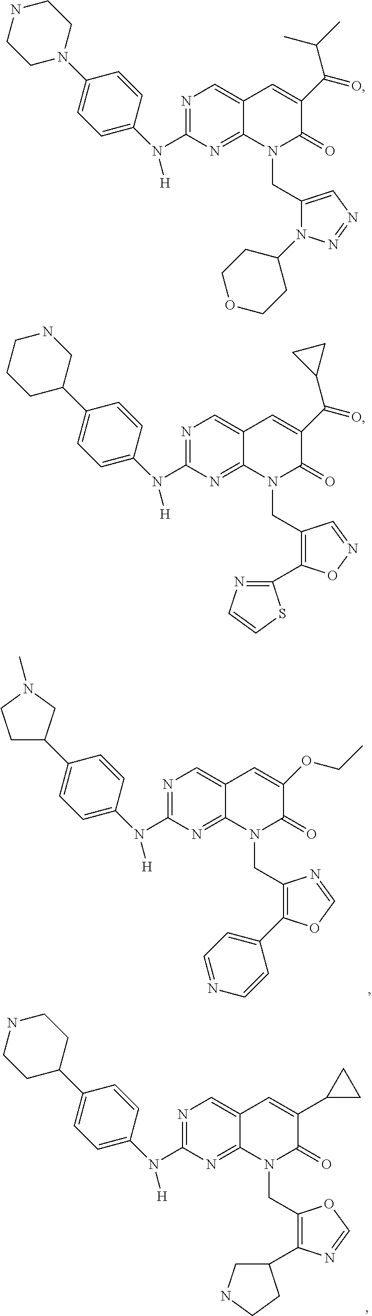 [SMILES: CC(C)C(=O)c1cc2cnc(Nc3ccc(N4CCNCC4)cc3)nc2n(Cc2cnnn2C2CCOCC2)c1=O ; O=C(c1cc2cnc(Nc3ccc(C4CCCNC4)cc3)nc2n(Cc2cnoc2-c2nccs2)c1=O)C1CC1 ; CCOc1cc2cnc(Nc3ccc(C4CCN(C)C4)cc3)nc2n(Cc2ncoc2-c2ccncc2)c1=O ; O=c1c(C2CC2)cc2cnc(Nc3ccc(C4CCNCC4)cc3)nc2n1Cc1ocnc1C1CCNC1]

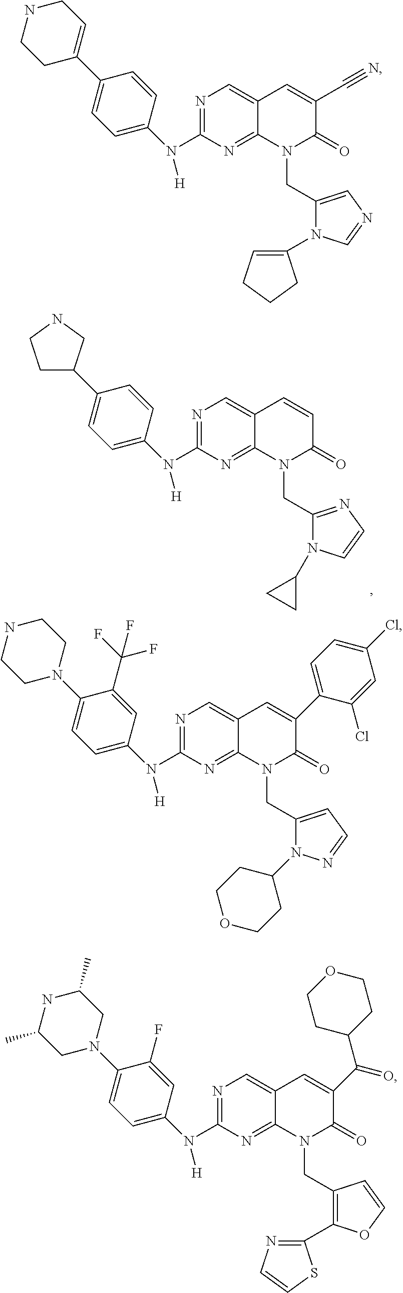 [SMILES: N#Cc1cc2cnc(Nc3ccc(C4=CCNCC4)cc3)nc2n(Cc2cncn2C2=CCCC2)c1=O ; O=c1ccc2cnc(Nc3ccc(C4CCNC4)cc3)nc2n1Cc1nccn1C1CC1 ; O=c1c(-c2ccc(Cl)cc2Cl)cc2cnc(Nc3ccc(N4CCNCC4)c(C(F)(F)F)c3)nc2n1Cc1ccnn1C1CCOCC1 ; C[C@@H]1CN(c2ccc(Nc3ncc4cc(C(=O)C5CCOCC5)c(=O)n(Cc5ccoc5-c5nccs5)c4n3)cc2F)C[C@H](C)N1]

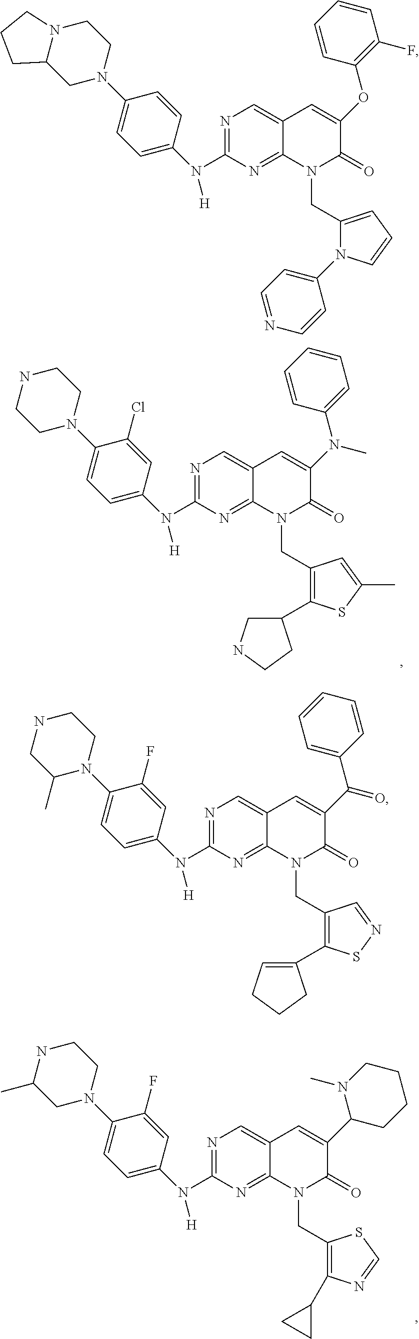

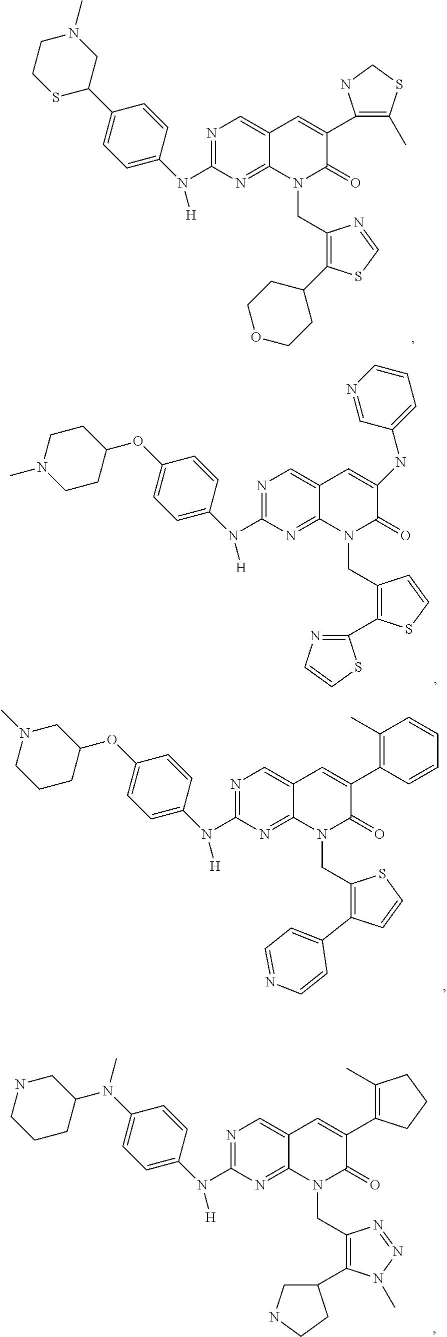

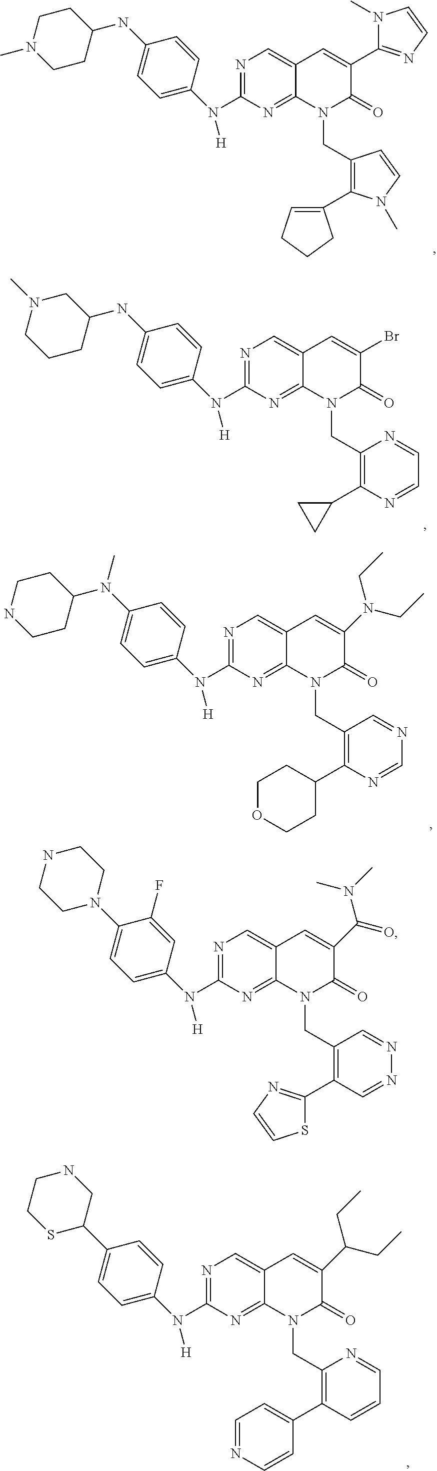 [SMILES: CN1CCC(Nc2ccc(Nc3ncc4cc(-c5nccn5C)c(=O)n(Cc5ccn(C)c5C5=CCCC5)c4n3)cc2)CC1 ; CN1CCCC(Nc2ccc(Nc3ncc4cc(Br)c(=O)n(Cc5nccnc5C5CC5)c4n3)cc2)C1 ; CCN(CC)c1cc2cnc(Nc3ccc(N(C)C4CCNCC4)cc3)nc2n(Cc2cncnc2C2CCOCC2)c1=O ; CN(C)C(=O)c1cc2cnc(Nc3ccc(N4CCNCC4)c(F)c3)nc2n(Cc2cnncc2-c2nccs2)c1=O ; CCC(CC)c1cc2cnc(Nc3ccc(C4CNCCS4)cc3)nc2n(Cc2ncccc2-c2ccncc2)c1=O]

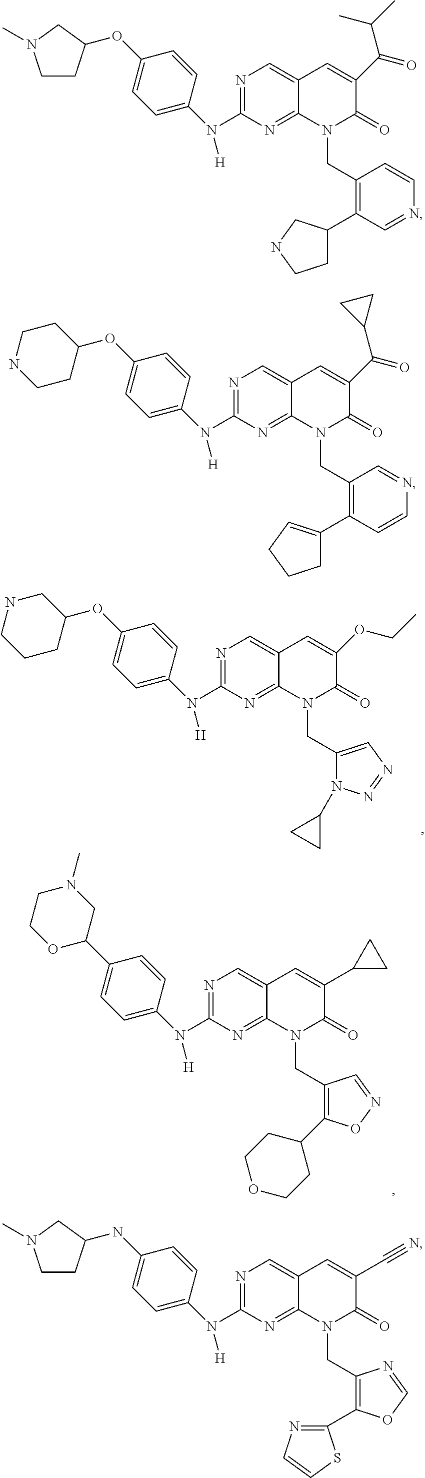

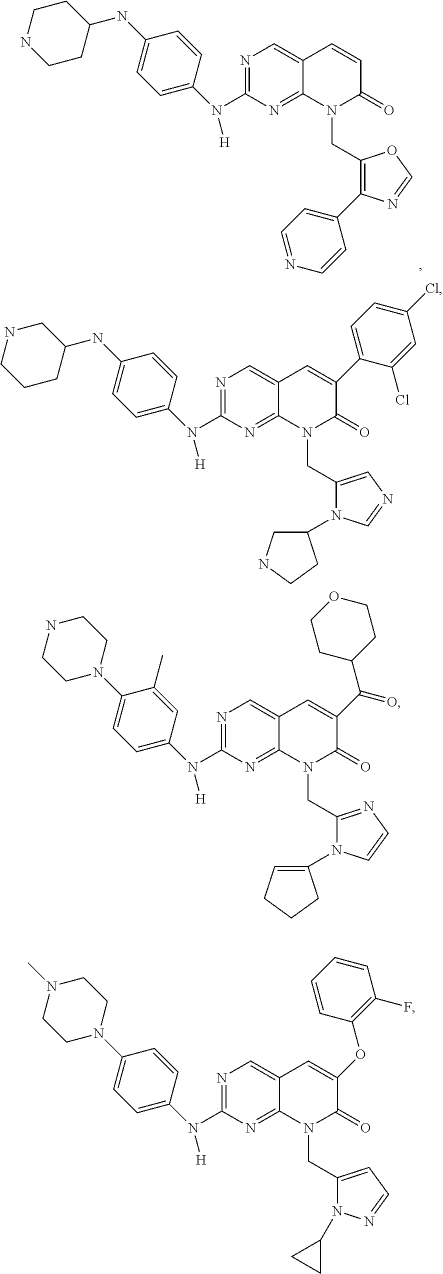

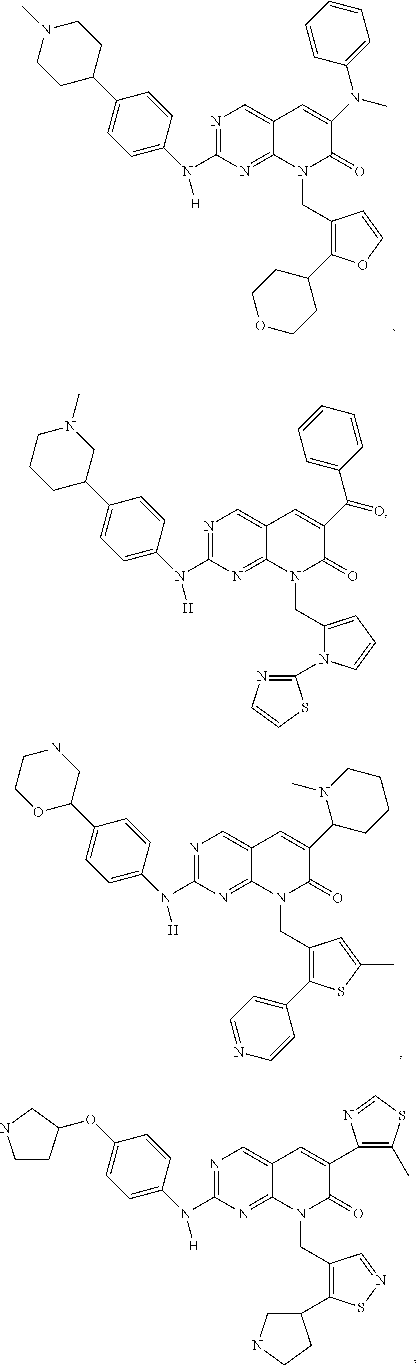

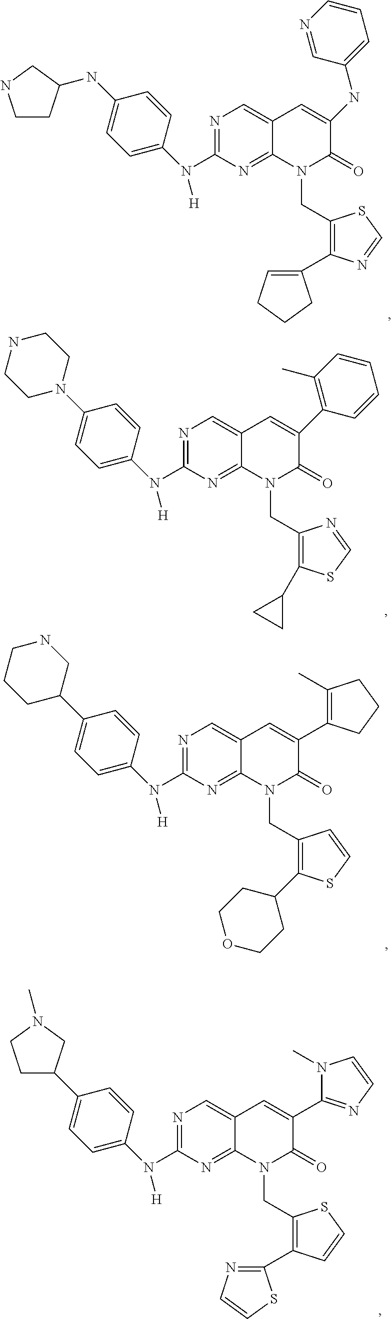

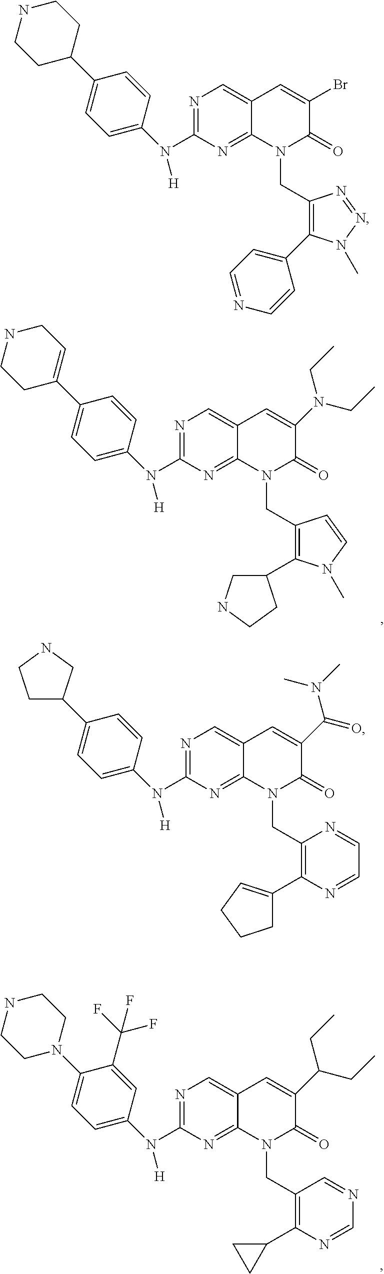 [SMILES: Cn1nnc(Cn2c(=O)c(Br)cc3cnc(Nc4ccc(C5CCNCC5)cc4)nc32)c1-c1ccncc1 ; CCN(CC)c1cc2cnc(Nc3ccc(C4=CCNCC4)cc3)nc2n(Cc2ccn(C)c2C2CCNC2)c1=O ; CN(C)C(=O)c1cc2cnc(Nc3ccc(C4CCNC4)cc3)nc2n(Cc2nccnc2C2=CCCC2)c1=O ; CCC(CC)c1cc2cnc(Nc3ccc(N4CCNCC4)c(C(F)(F)F)c3)nc2n(Cc2cncnc2C2CC2)c1=O]

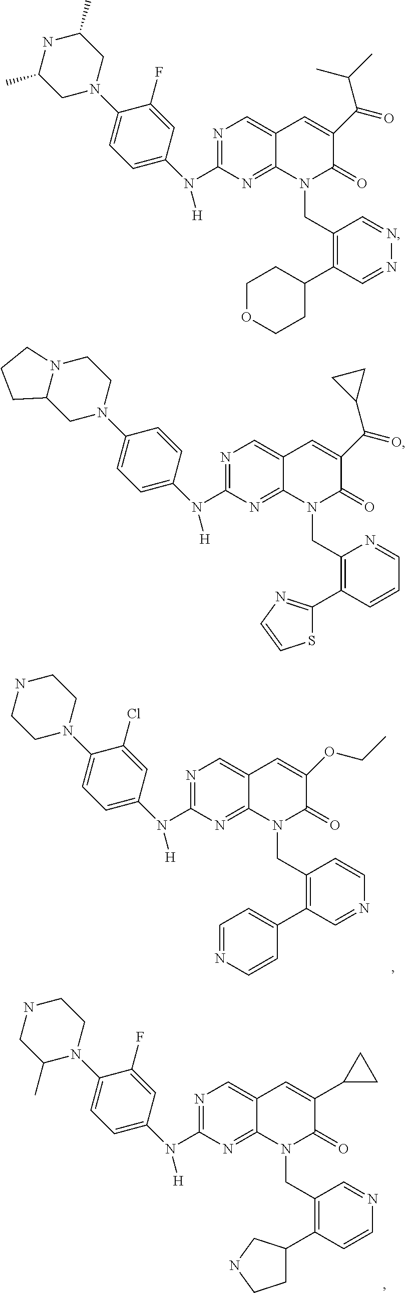 [SMILES: CC(C)C(=O)c1cc2cnc(Nc3ccc(N4C[C@@H](C)N[C@@H](C)C4)c(F)c3)nc2n(Cc2cnncc2C2CCOCC2)c1=O ; O=C(c1cc2cnc(Nc3ccc(N4CCN5CCCC5C4)cc3)nc2n(Cc2ncccc2-c2nccs2)c1=O)C1CC1 ; CCOc1cc2cnc(Nc3ccc(N4CCNCC4)c(Cl)c3)nc2n(Cc2ccncc2-c2ccncc2)c1=O ; CC1CNCCN1c1ccc(Nc2ncc3cc(C4CC4)c(=O)n(Cc4cnccc4C4CCNC4)c3n2)cc1F]

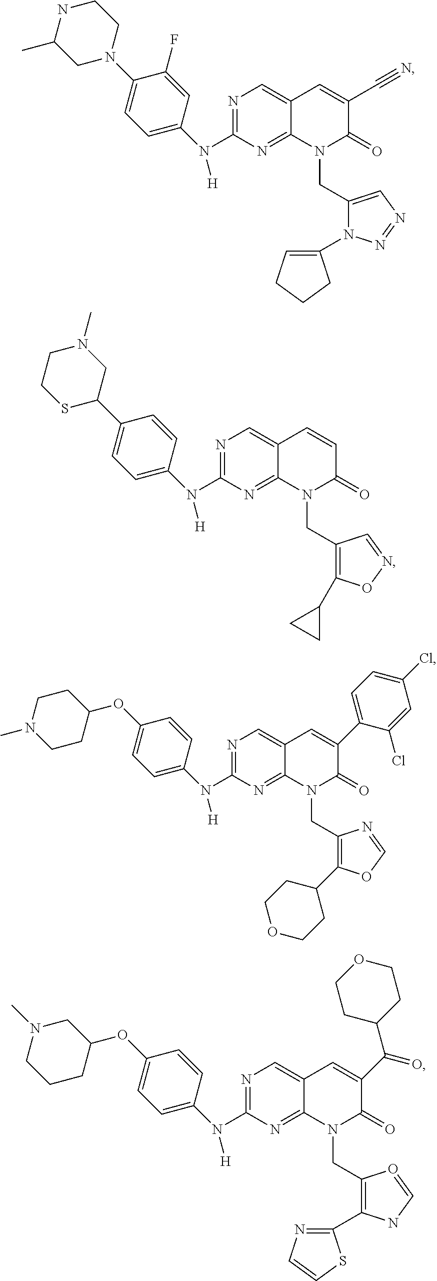

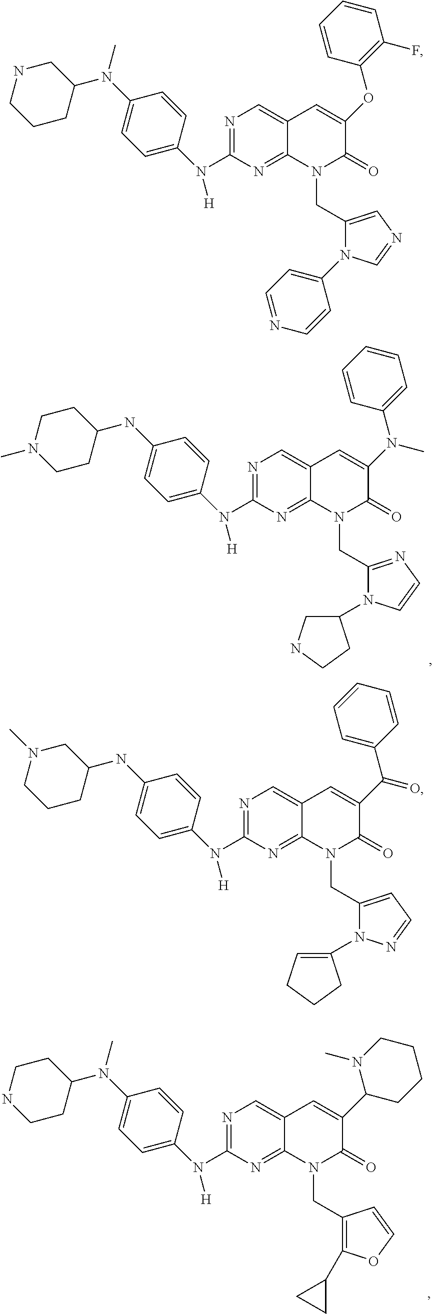

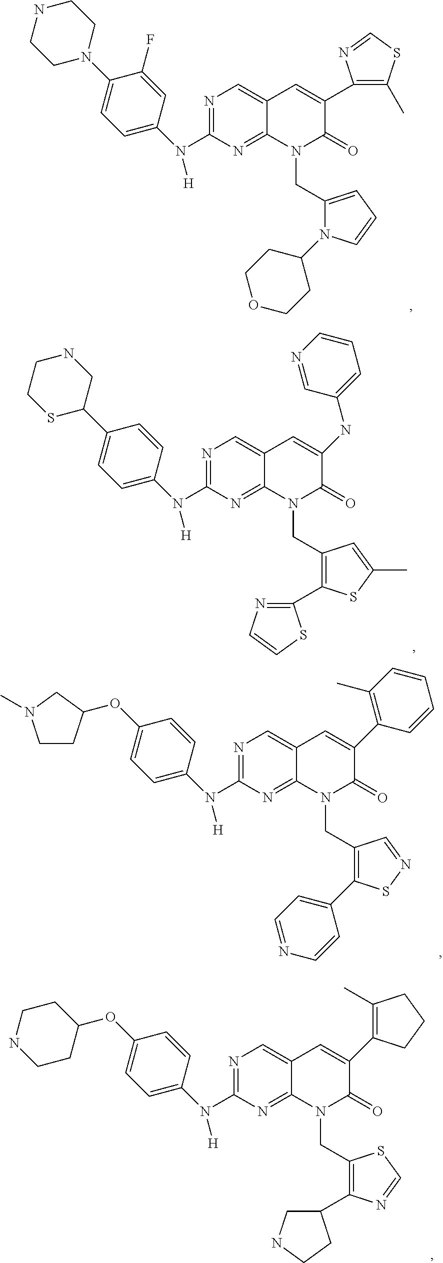

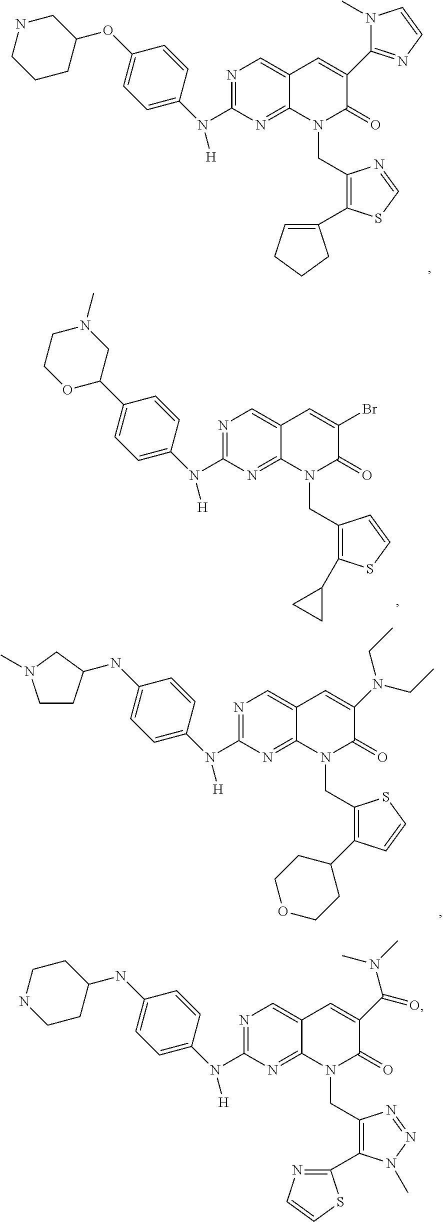

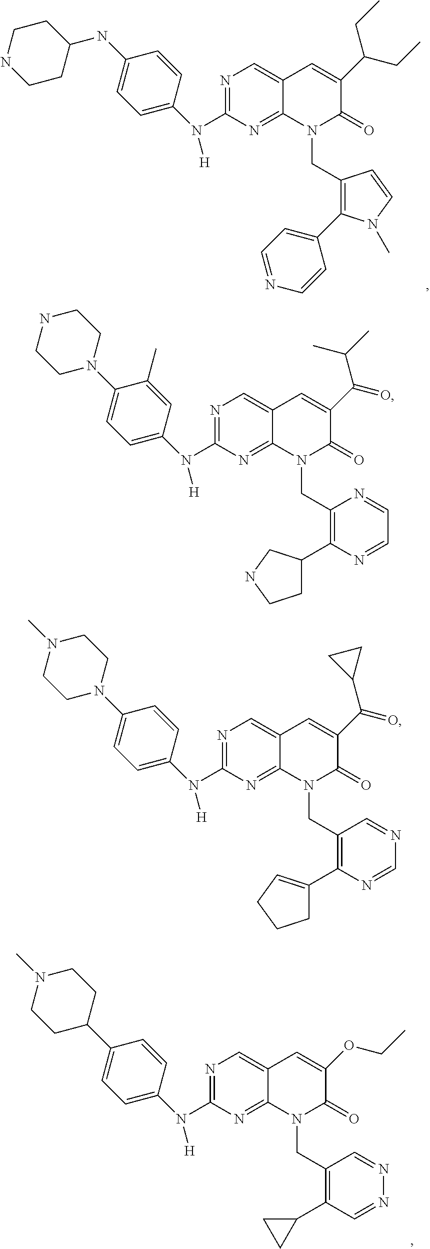

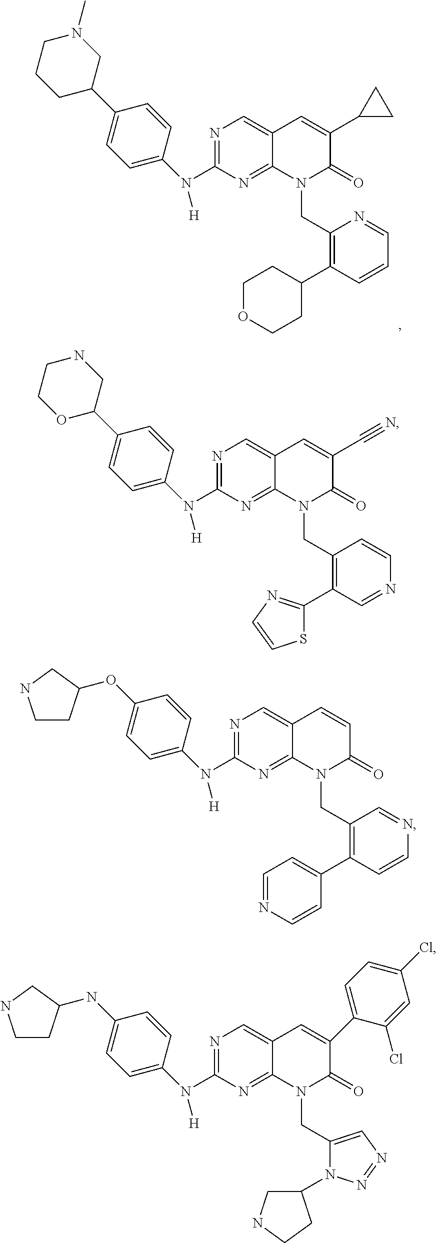 [SMILES: CN1CCCC(c2ccc(Nc3ncc4cc(C5CC5)c(=O)n(Cc5ncccc5C5CCOCC5)c4n3)cc2)C1 ; N#Cc1cc2cnc(Nc3ccc(C4CNCCO4)cc3)nc2n(Cc2ccncc2-c2nccs2)c1=O ; O=c1ccc2cnc(Nc3ccc(OC4CCNC4)cc3)nc2n1Cc1cnccc1-c1ccncc1 ; O=c1c(-c2ccc(Cl)cc2Cl)cc2cnc(Nc3ccc(NC4CCNC4)cc3)nc2n1Cc1cnnn1C1CCNC1]

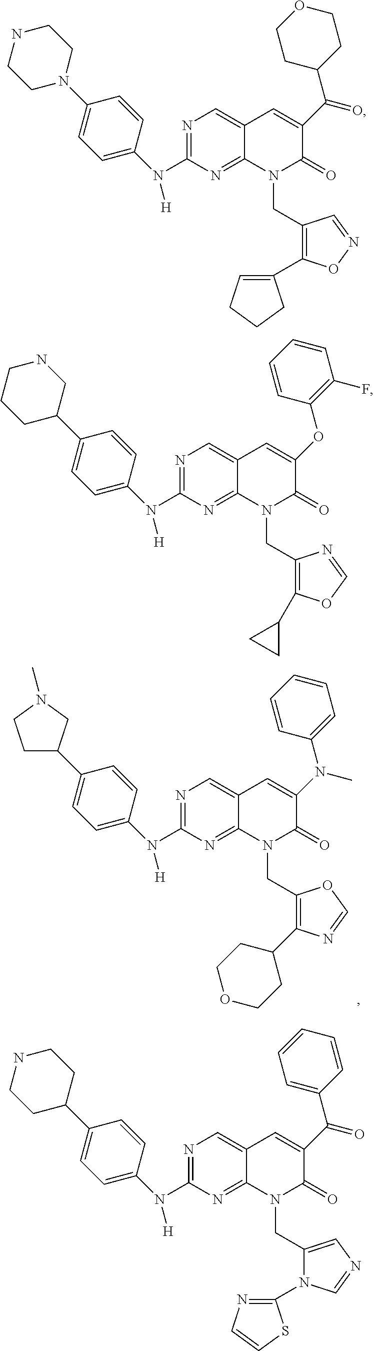

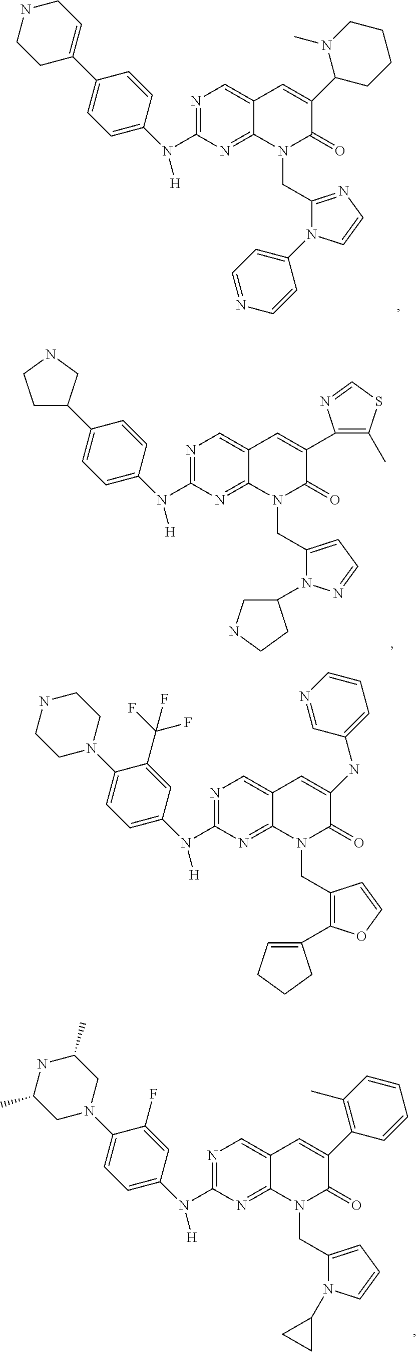

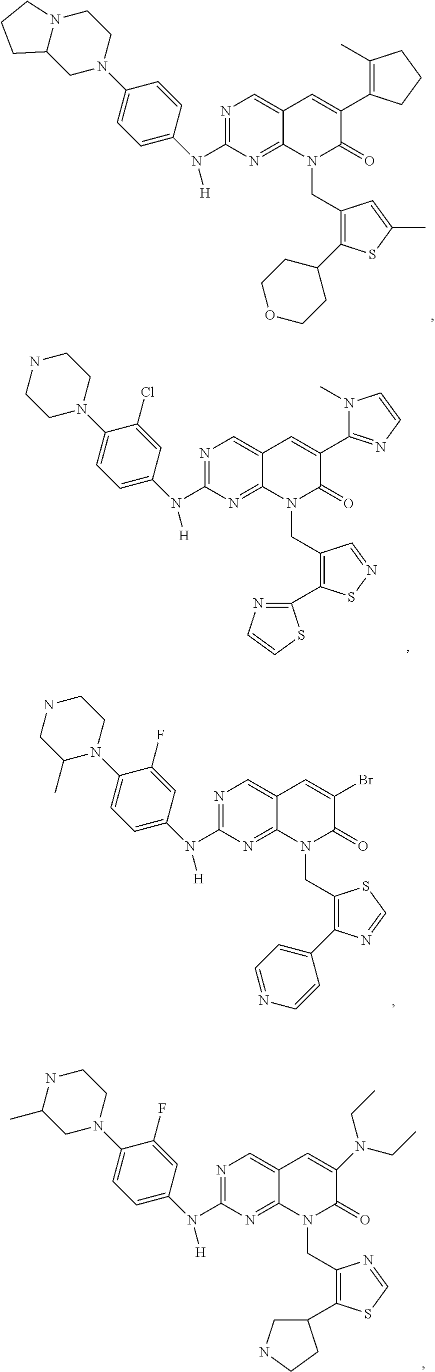

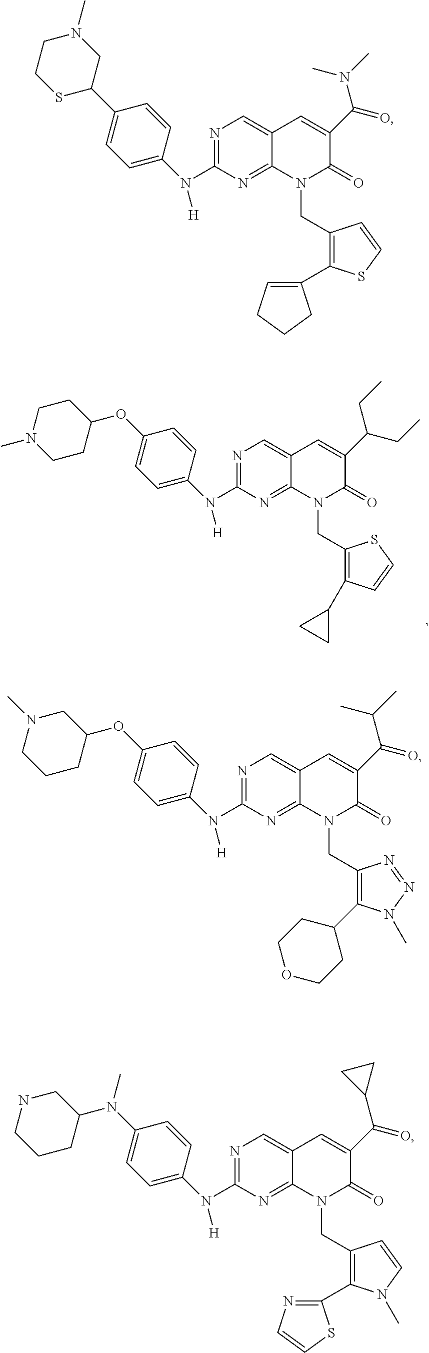

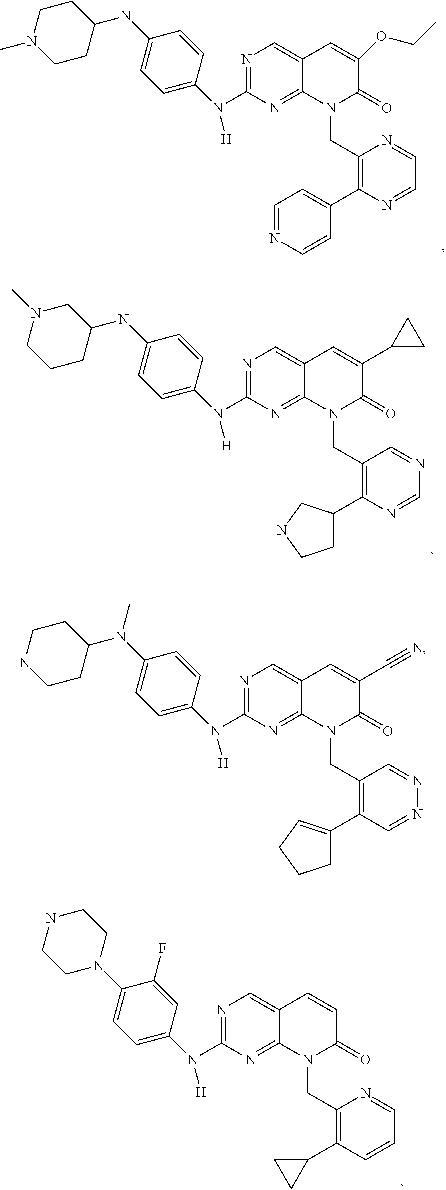

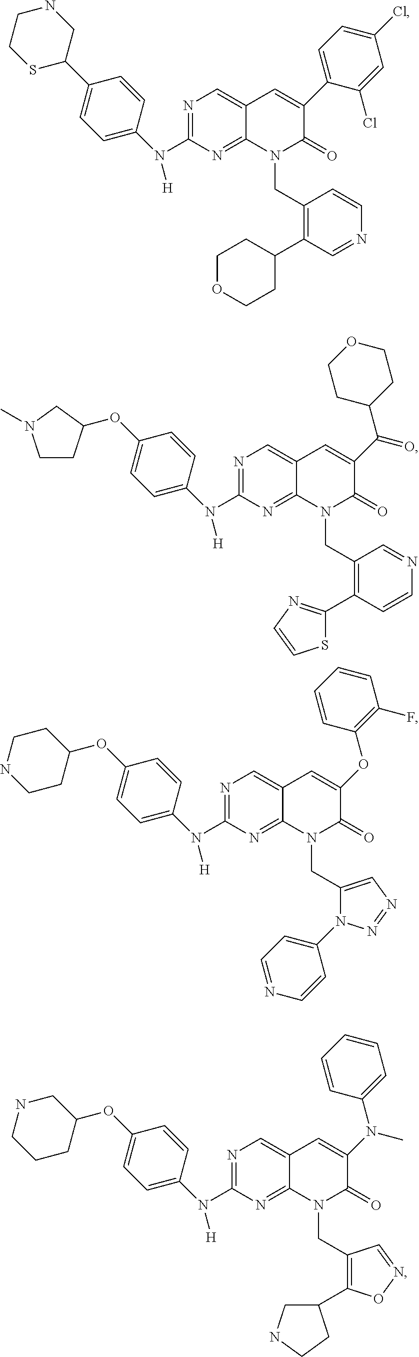

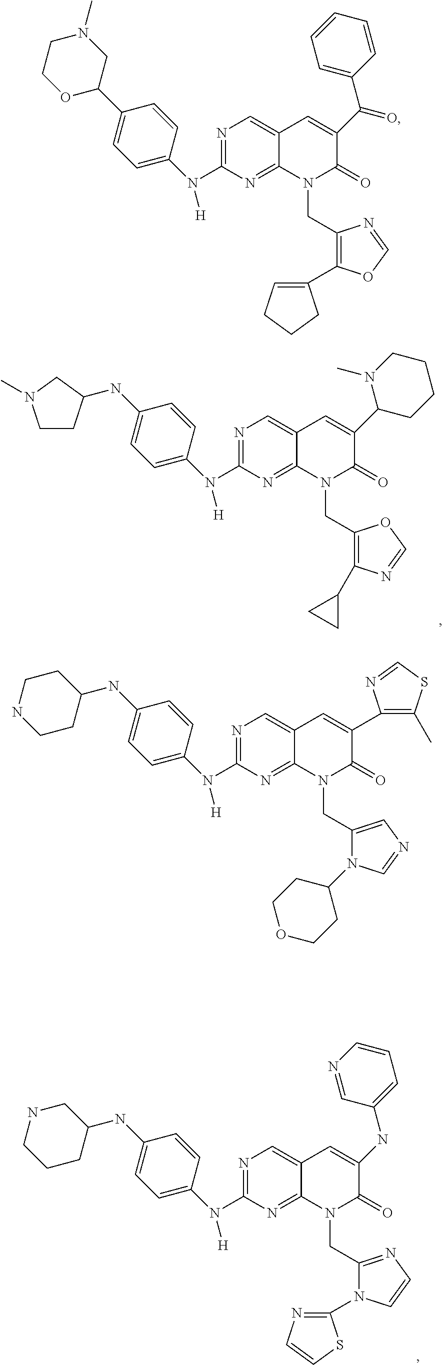 [SMILES: CN1CCOC(c2ccc(Nc3ncc4cc(C(=O)c5ccccc5)c(=O)n(Cc5ncoc5C5=CCCC5)c4n3)cc2)C1 ; CN1CCC(Nc2ccc(Nc3ncc4cc(C5CCCCN5C)c(=O)n(Cc5ocnc5C5CC5)c4n3)cc2)C1 ; Cc1scnc1-c1cc2cnc(Nc3ccc(NC4CCNCC4)cc3)nc2n(Cc2cncn2C2CCOCC2)c1=O ; O=c1c(Nc2cccnc2)cc2cnc(Nc3ccc(NC4CCCNC4)cc3)nc2n1Cc1nccn1-c1nccs1]

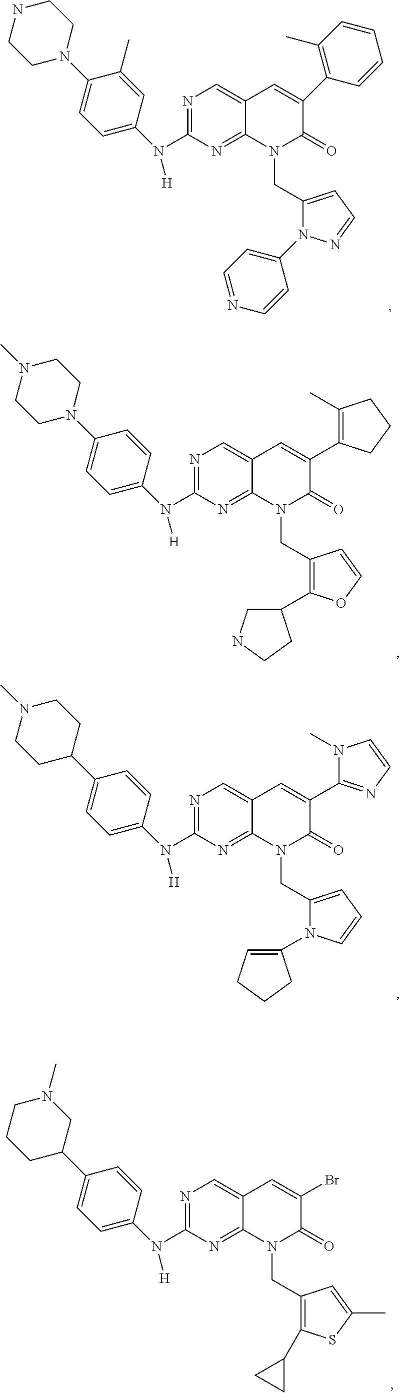 [SMILES: Cc1ccccc1-c1cc2cnc(Nc3ccc(N4CCNCC4)c(C)c3)nc2n(Cc2ccnn2-c2ccncc2)c1=O ; CC1=C(c2cc3cnc(Nc4ccc(N5CCN(C)CC5)cc4)nc3n(Cc3ccoc3C3CCNC3)c2=O)CCC1 ; CN1CCC(c2ccc(Nc3ncc4cc(-c5nccn5C)c(=O)n(Cc5cccn5C5=CCCC5)c4n3)cc2)CC1 ; Cc1cc(Cn2c(=O)c(Br)cc3cnc(Nc4ccc(C5CCCN(C)C5)cc4)nc32)c(C2CC2)s1]

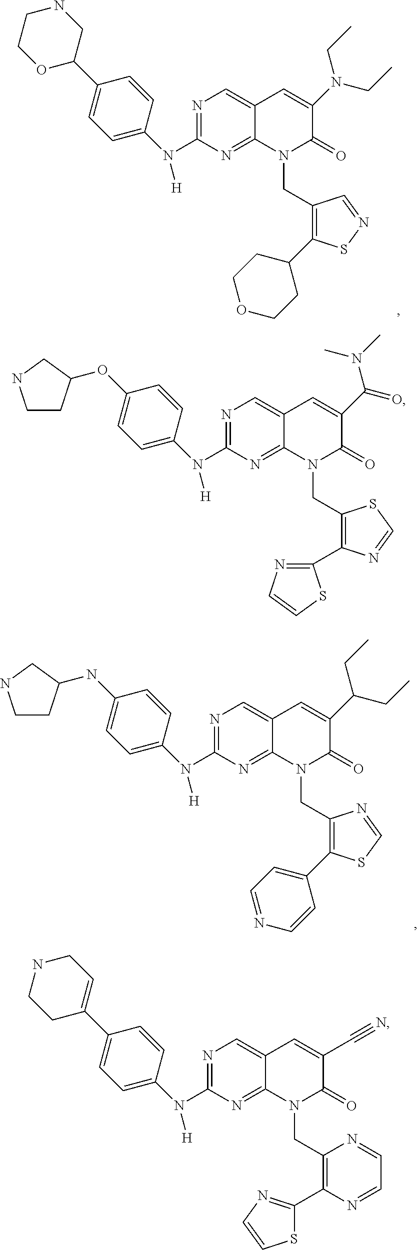 [SMILES: CCN(CC)c1cc2cnc(Nc3ccc(C4CNCCO4)cc3)nc2n(Cc2cnsc2C2CCOCC2)c1=O ; CN(C)C(=O)c1cc2cnc(Nc3ccc(OC4CCNC4)cc3)nc2n(Cc2scnc2-c2nccs2)c1=O ; CCC(CC)c1cc2cnc(Nc3ccc(NC4CCNC4)cc3)nc2n(Cc2ncsc2-c2ccncc2)c1=O ; N#Cc1cc2cnc(Nc3ccc(C4=CCNCC4)cc3)nc2n(Cc2nccnc2-c2nccs2)c1=O]

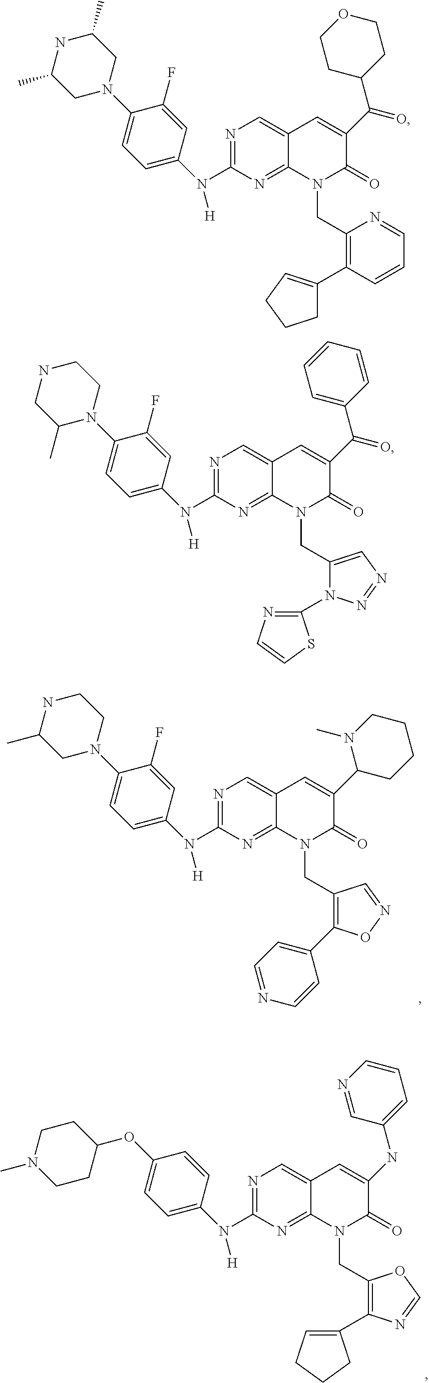

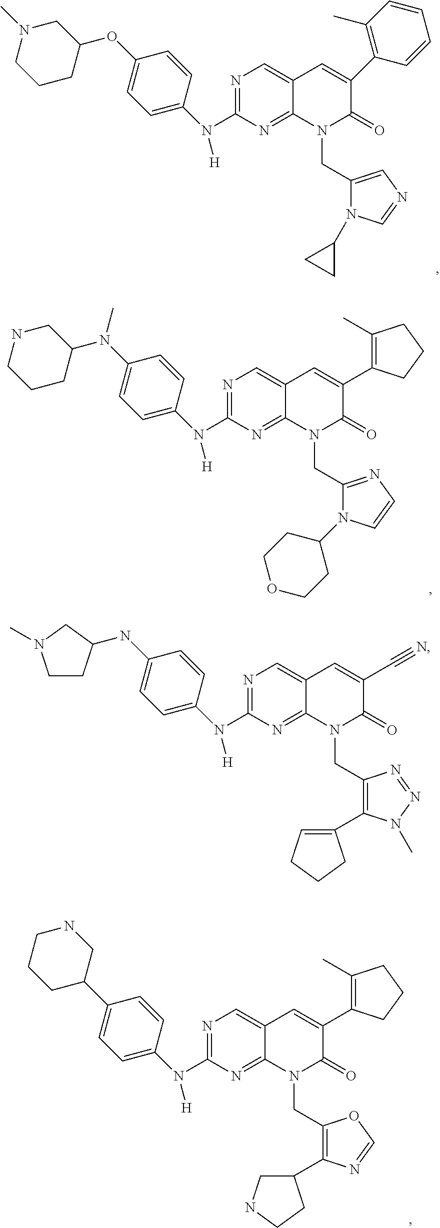

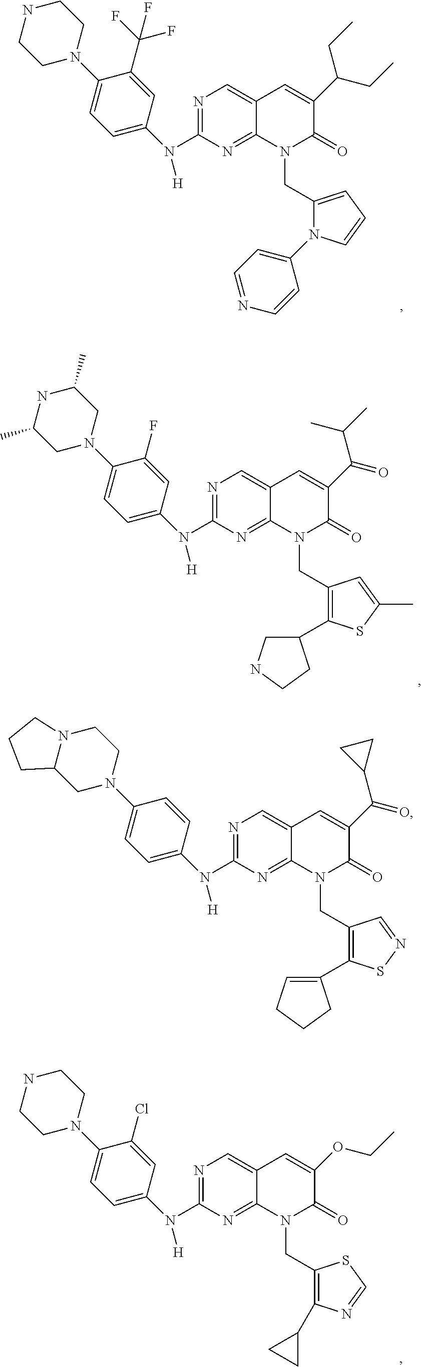

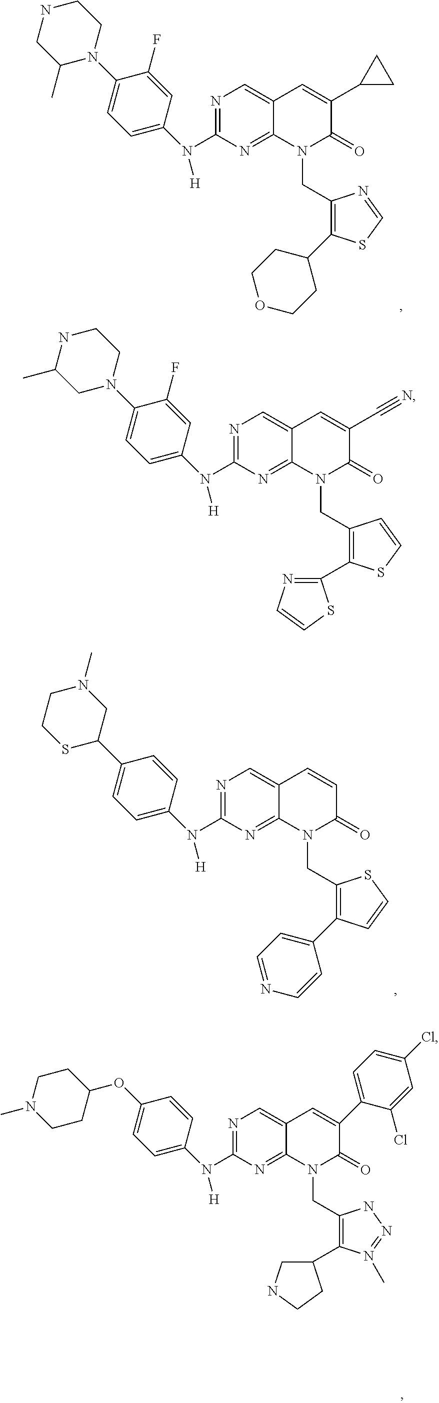

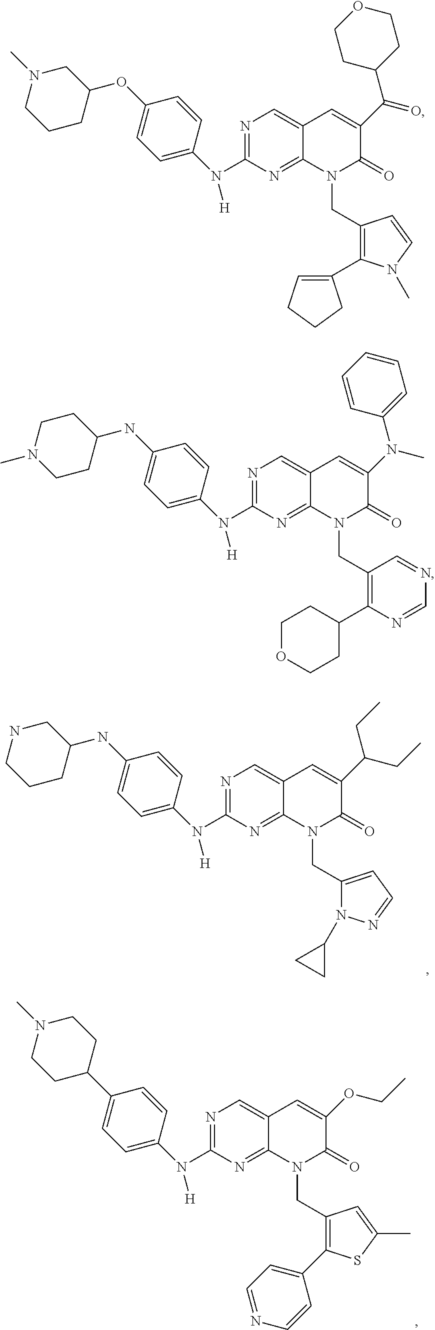

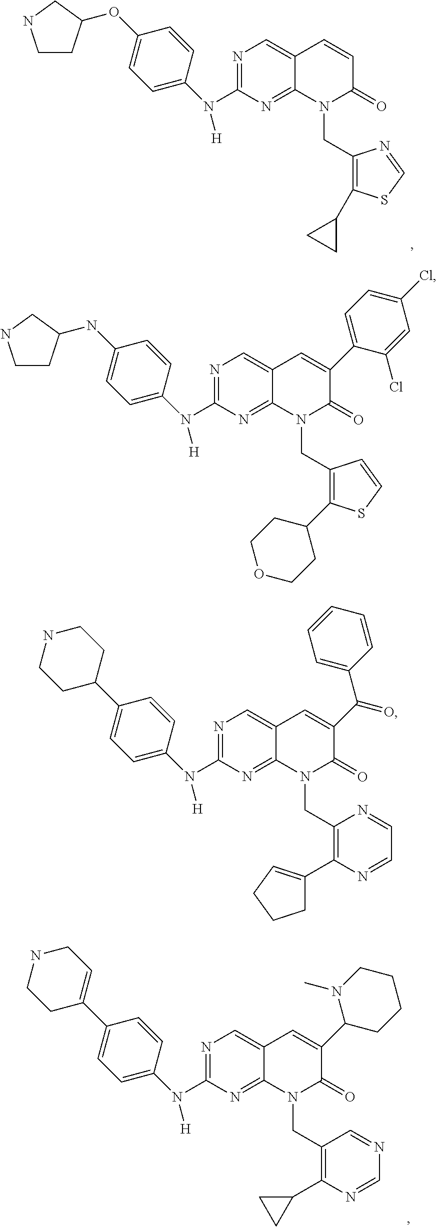 [SMILES: O=c1ccc2cnc(Nc3ccc(OC4CCNC4)cc3)nc2n1Cc1ncsc1C1CC1 ; O=c1c(-c2ccc(Cl)cc2Cl)cc2cnc(Nc3ccc(NC4CCNC4)cc3)nc2n1Cc1ccsc1C1CCOCC1 ; O=C(c1ccccc1)c1cc2cnc(Nc3ccc(C4CCNCC4)cc3)nc2n(Cc2nccnc2C2=CCCC2)c1=O ; CN1CCCCC1c1cc2cnc(Nc3ccc(C4=CCNCC4)cc3)nc2n(Cc2cncnc2C2CC2)c1=O]

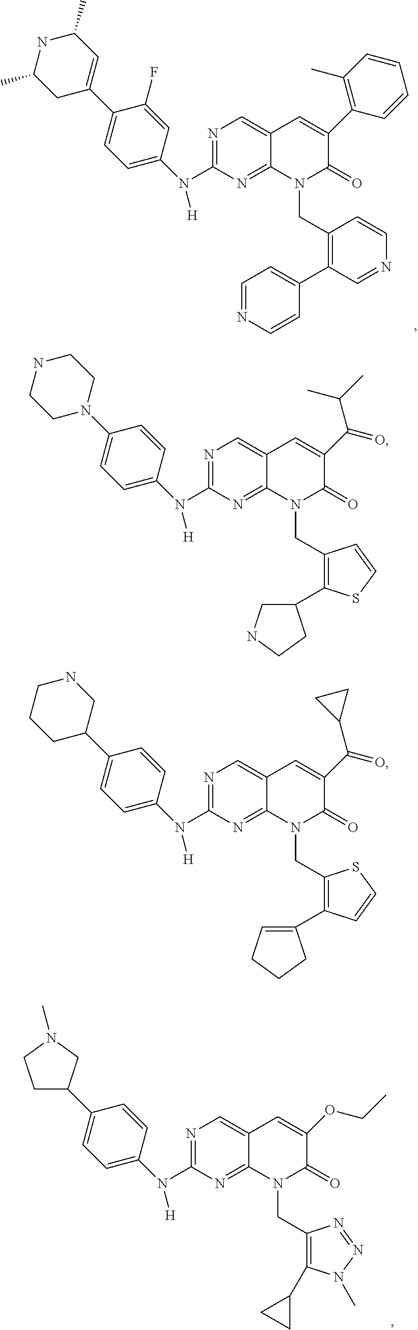 [SMILES: Cc1ccccc1-c1cc2cnc(Nc3ccc(C4=C[C@@H](C)N[C@@H](C)C4)c(F)c3)nc2n(Cc2ccncc2-c2ccncc2)c1=O ; CC(C)C(=O)c1cc2cnc(Nc3ccc(N4CCNCC4)cc3)nc2n(Cc2ccsc2C2CCNC2)c1=O ; O=C(c1cc2cnc(Nc3ccc(C4CCCNC4)cc3)nc2n(Cc2sccc2C2=CCCC2)c1=O)C1CC1 ; CCOc1cc2cnc(Nc3ccc(C4CCN(C)C4)cc3)nc2n(Cc2nnn(C)c2C2CC2)c1=O]

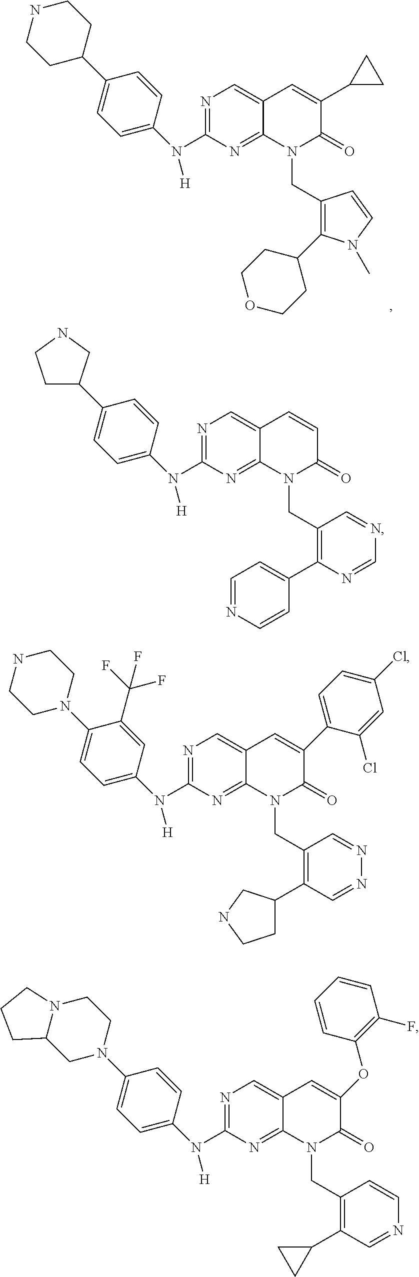 [SMILES: Cn1ccc(Cn2c(=O)c(C3CC3)cc3cnc(Nc4ccc(C5CCNCC5)cc4)nc32)c1C1CCOCC1 ; O=c1ccc2cnc(Nc3ccc(C4CCNC4)cc3)nc2n1Cc1cncnc1-c1ccncc1 ; O=c1c(-c2ccc(Cl)cc2Cl)cc2cnc(Nc3ccc(N4CCNCC4)c(C(F)(F)F)c3)nc2n1Cc1cnncc1C1CCNC1 ; O=c1c(Oc2ccccc2F)cc2cnc(Nc3ccc(N4CCN5CCCC5C4)cc3)nc2n1Cc1ccncc1C1CC1]

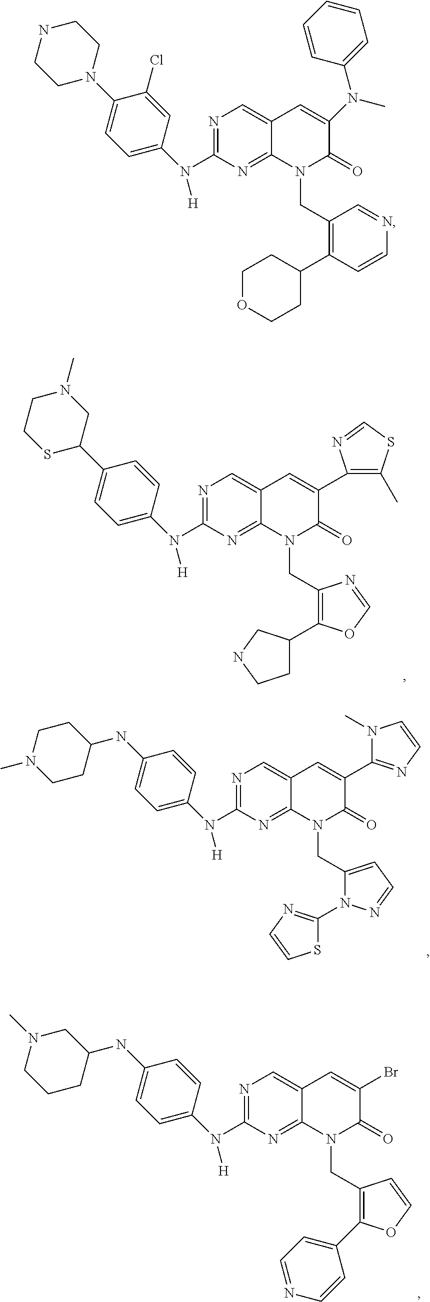

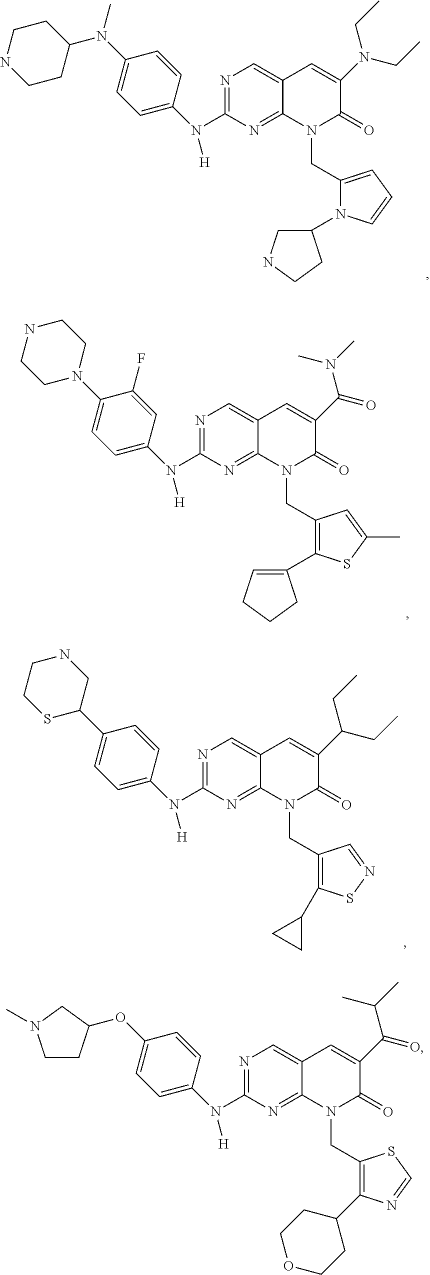

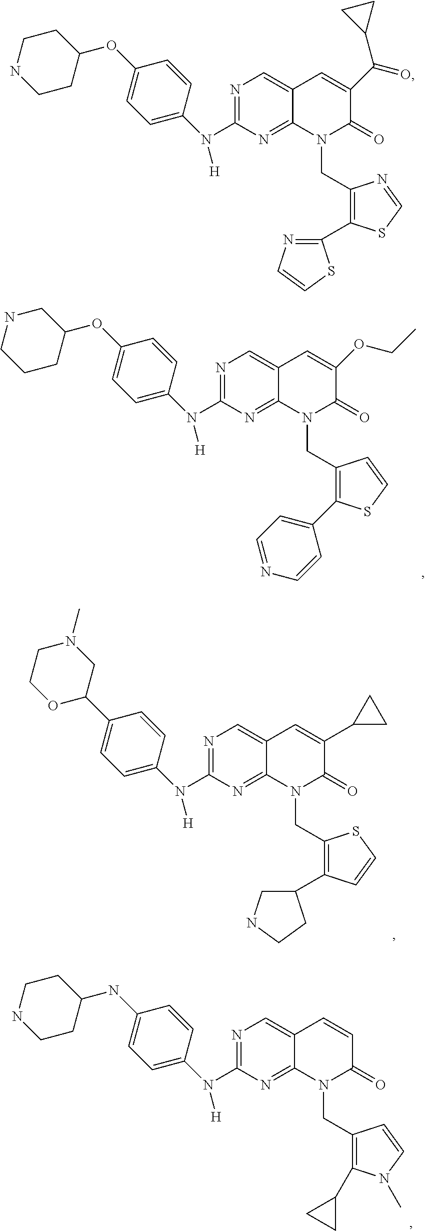 [SMILES: O=C(c1cc2cnc(Nc3ccc(OC4CCNCC4)cc3)nc2n(Cc2ncsc2-c2nccs2)c1=O)C1CC1 ; CCOc1cc2cnc(Nc3ccc(OC4CCCNC4)cc3)nc2n(Cc2ccsc2-c2ccncc2)c1=O ; CN1CCOC(c2ccc(Nc3ncc4cc(C5CC5)c(=O)n(Cc5sccc5C5CCNC5)c4n3)cc2)C1 ; Cn1ccc(Cn2c(=O)ccc3cnc(Nc4ccc(NC5CCNCC5)cc4)nc32)c1C1CC1]

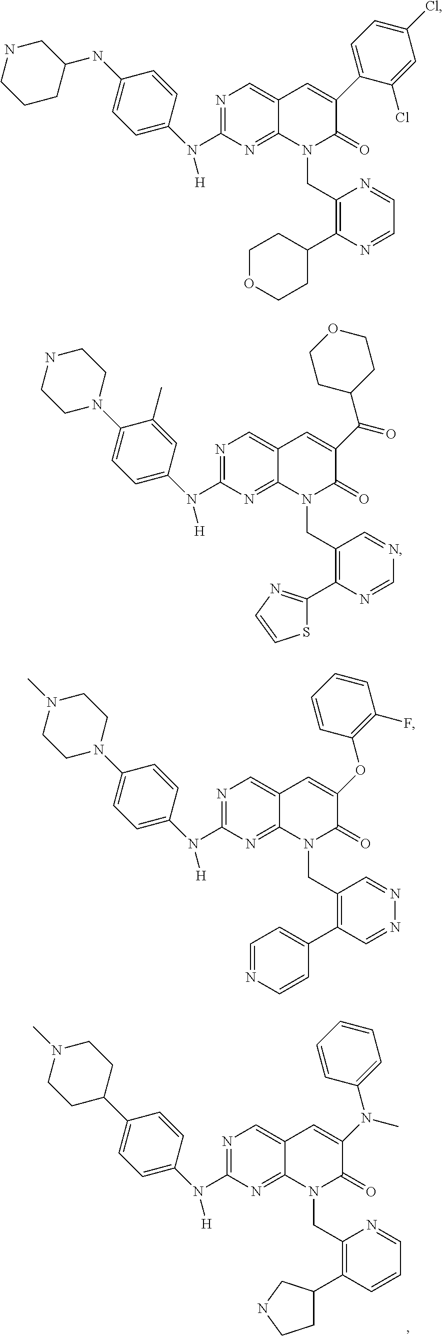 [SMILES: O=c1c(-c2ccc(Cl)cc2Cl)cc2cnc(Nc3ccc(NC4CCCNC4)cc3)nc2n1Cc1nccnc1C1CCOCC1 ; Cc1cc(Nc2ncc3cc(C(=O)C4CCOCC4)c(=O)n(Cc4cncnc4-c4nccs4)c3n2)ccc1N1CCNCC1 ; CN1CCN(c2ccc(Nc3ncc4cc(Oc5ccccc5F)c(=O)n(Cc5cnncc5-c5ccncc5)c4n3)cc2)CC1 ; CN1CCC(c2ccc(Nc3ncc4cc(N(C)c5ccccc5)c(=O)n(Cc5ncccc5C5CCNC5)c4n3)cc2)CC1]

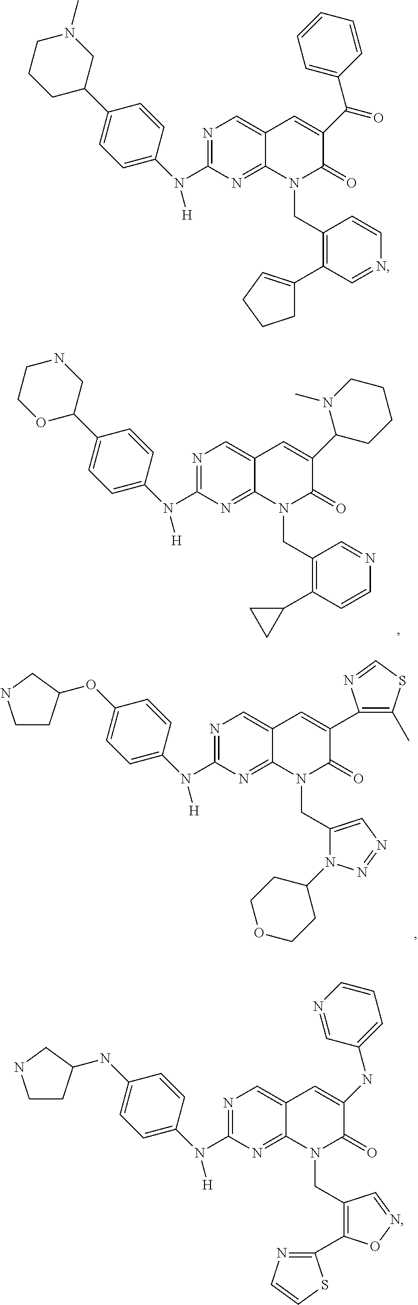 [SMILES: CN1CCCC(c2ccc(Nc3ncc4cc(C(=O)c5ccccc5)c(=O)n(Cc5ccncc5C5=CCCC5)c4n3)cc2)C1 ; CN1CCCCC1c1cc2cnc(Nc3ccc(C4CNCCO4)cc3)nc2n(Cc2cnccc2C2CC2)c1=O ; Cc1scnc1-c1cc2cnc(Nc3ccc(OC4CCNC4)cc3)nc2n(Cc2cnnn2C2CCOCC2)c1=O ; O=c1c(Nc2cccnc2)cc2cnc(Nc3ccc(NC4CCNC4)cc3)nc2n1Cc1cnoc1-c1nccs1]

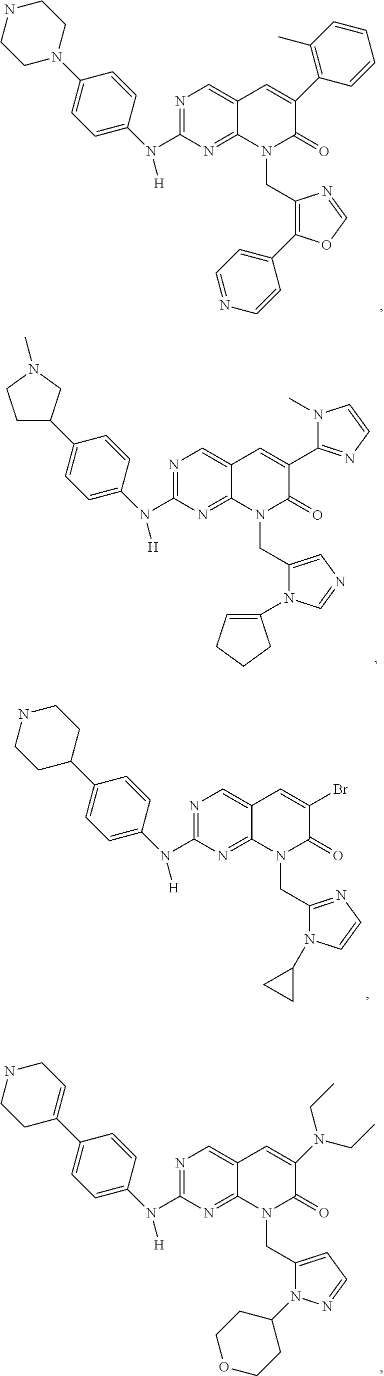 [SMILES: Cc1ccccc1-c1cc2cnc(Nc3ccc(N4CCNCC4)cc3)nc2n(Cc2ncoc2-c2ccncc2)c1=O ; CN1CCC(c2ccc(Nc3ncc4cc(-c5nccn5C)c(=O)n(Cc5cncn5C5=CCCC5)c4n3)cc2)C1 ; O=c1c(Br)cc2cnc(Nc3ccc(C4CCNCC4)cc3)nc2n1Cc1nccn1C1CC1 ; CCN(CC)c1cc2cnc(Nc3ccc(C4=CCNCC4)cc3)nc2n(Cc2ccnn2C2CCOCC2)c1=O]

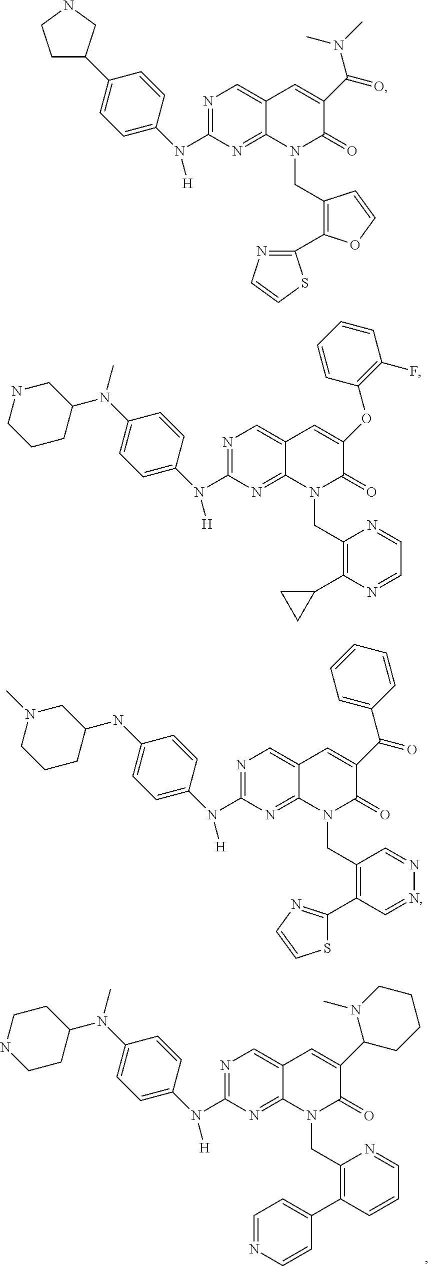 [SMILES: CN(C)C(=O)c1cc2cnc(Nc3ccc(C4CCNC4)cc3)nc2n(Cc2ccoc2-c2nccs2)c1=O ; CN(c1ccc(Nc2ncc3cc(Oc4ccccc4F)c(=O)n(Cc4nccnc4C4CC4)c3n2)cc1)C1CCCNC1 ; CN1CCCC(Nc2ccc(Nc3ncc4cc(C(=O)c5ccccc5)c(=O)n(Cc5cnncc5-c5nccs5)c4n3)cc2)C1 ; CN1CCCCC1c1cc2cnc(Nc3ccc(N(C)C4CCNCC4)cc3)nc2n(Cc2ncccc2-c2ccncc2)c1=O]

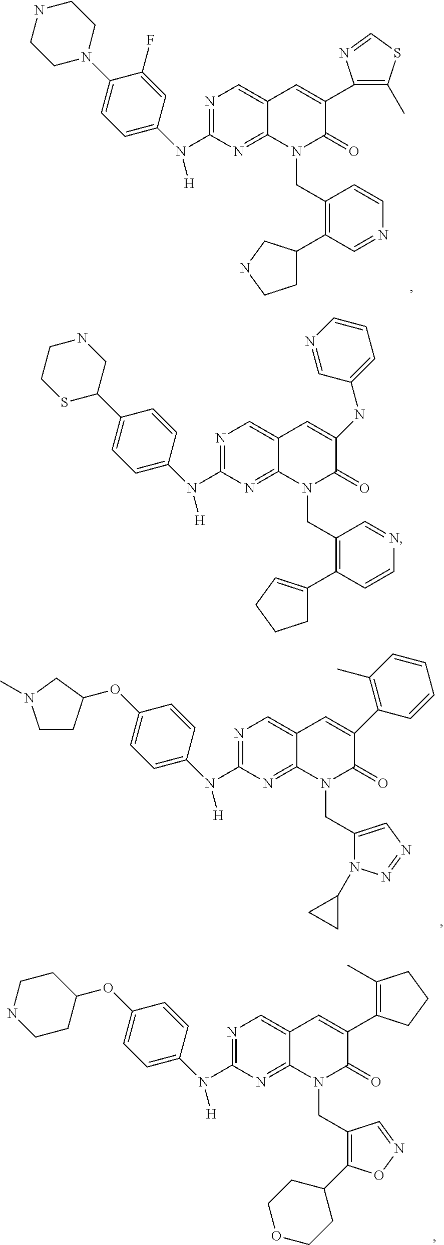

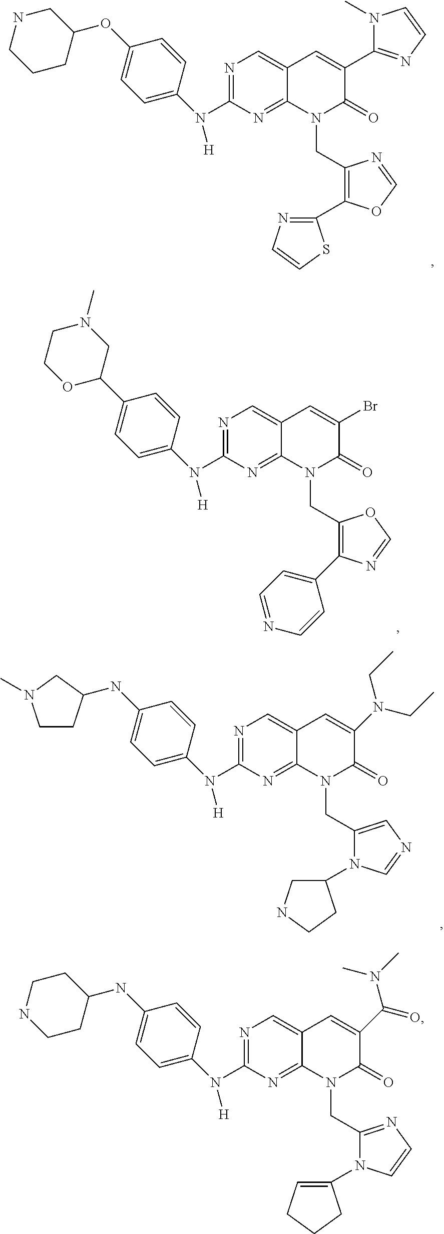 [SMILES: Cn1ccnc1-c1cc2cnc(Nc3ccc(OC4CCCNC4)cc3)nc2n(Cc2ncoc2-c2nccs2)c1=O ; CN1CCOC(c2ccc(Nc3ncc4cc(Br)c(=O)n(Cc5ocnc5-c5ccncc5)c4n3)cc2)C1 ; CCN(CC)c1cc2cnc(Nc3ccc(NC4CCN(C)C4)cc3)nc2n(Cc2cncn2C2CCNC2)c1=O ; CN(C)C(=O)c1cc2cnc(Nc3ccc(NC4CCNCC4)cc3)nc2n(Cc2nccn2C2=CCCC2)c1=O]

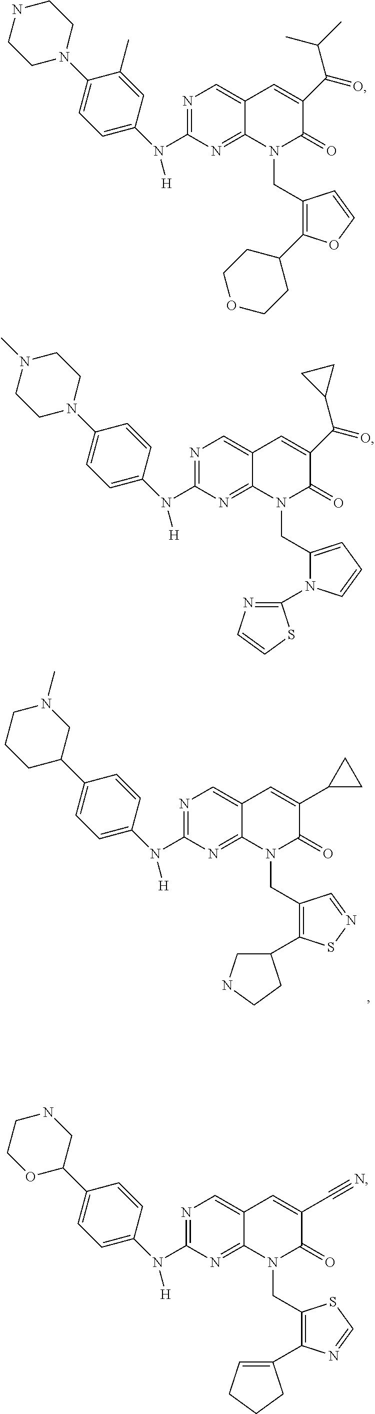 [SMILES: Cc1cc(Nc2ncc3cc(C(=O)C(C)C)c(=O)n(Cc4ccoc4C4CCOCC4)c3n2)ccc1N1CCNCC1 ; CN1CCN(c2ccc(Nc3ncc4cc(C(=O)C5CC5)c(=O)n(Cc5cccn5-c5nccs5)c4n3)cc2)CC1 ; CN1CCCC(c2ccc(Nc3ncc4cc(C5CC5)c(=O)n(Cc5cnsc5C5CCNC5)c4n3)cc2)C1 ; N#Cc1cc2cnc(Nc3ccc(C4CNCCO4)cc3)nc2n(Cc2scnc2C2=CCCC2)c1=O]

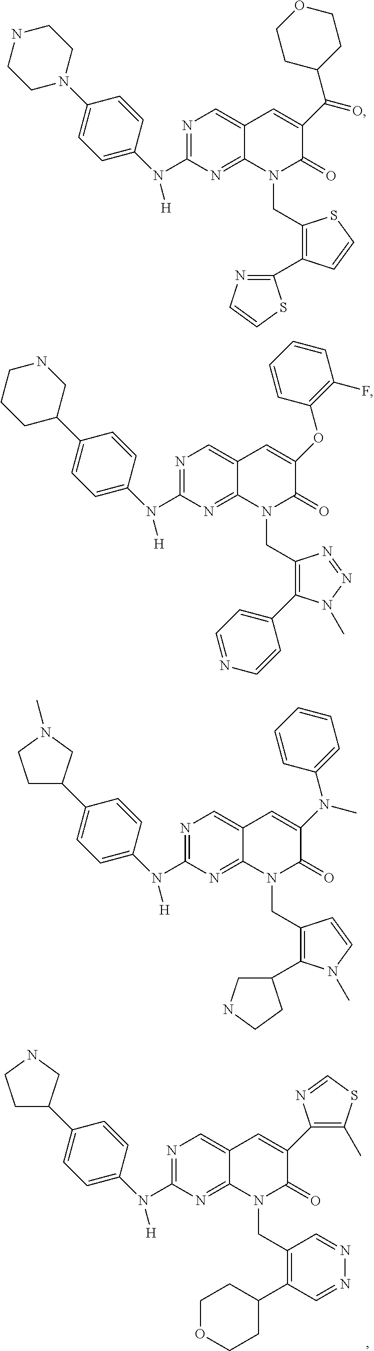

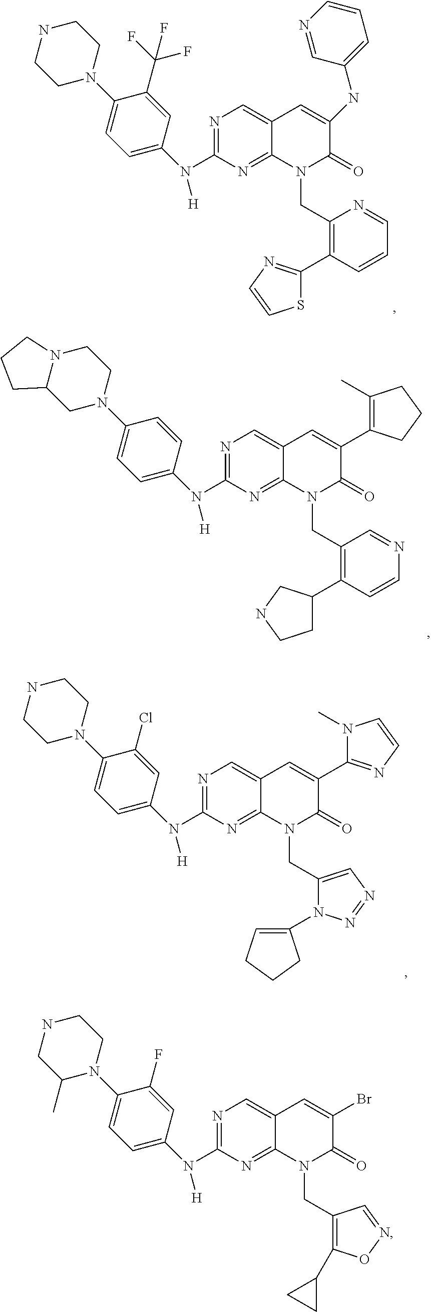 [SMILES: O=c1c(Nc2cccnc2)cc2cnc(Nc3ccc(N4CCNCC4)c(C(F)(F)F)c3)nc2n1Cc1ncccc1-c1nccs1 ; CC1=C(c2cc3cnc(Nc4ccc(N5CCN6CCCC6C5)cc4)nc3n(Cc3cnccc3C3CCNC3)c2=O)CCC1 ; Cn1ccnc1-c1cc2cnc(Nc3ccc(N4CCNCC4)c(Cl)c3)nc2n(Cc2cnnn2C2=CCCC2)c1=O ; CC1CNCCN1c1ccc(Nc2ncc3cc(Br)c(=O)n(Cc4cnoc4C4CC4)c3n2)cc1F]

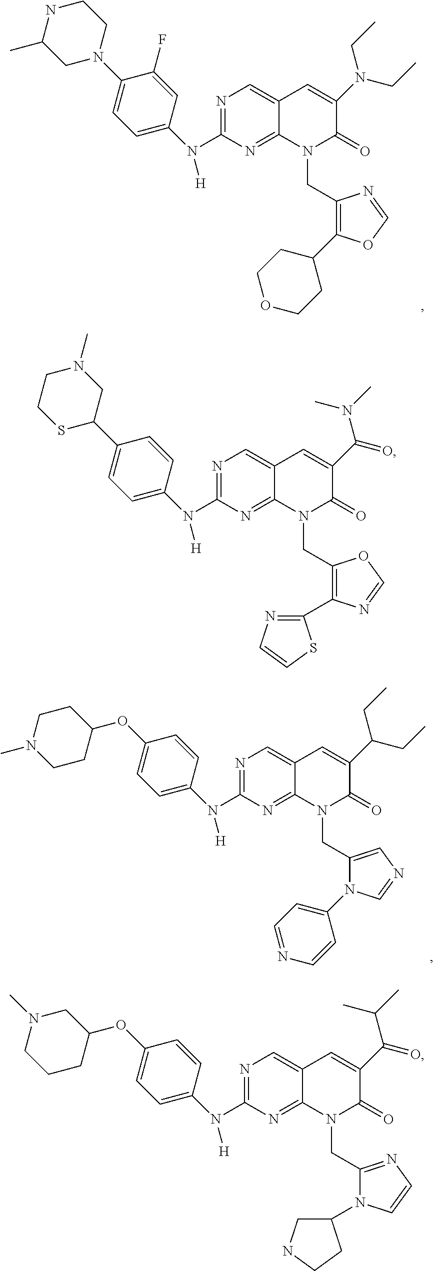

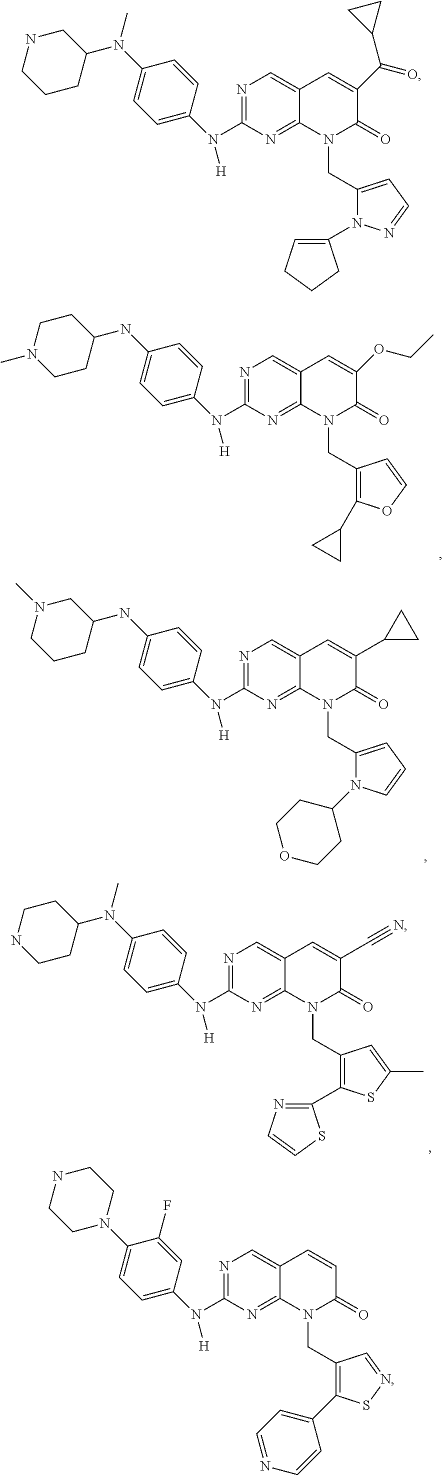

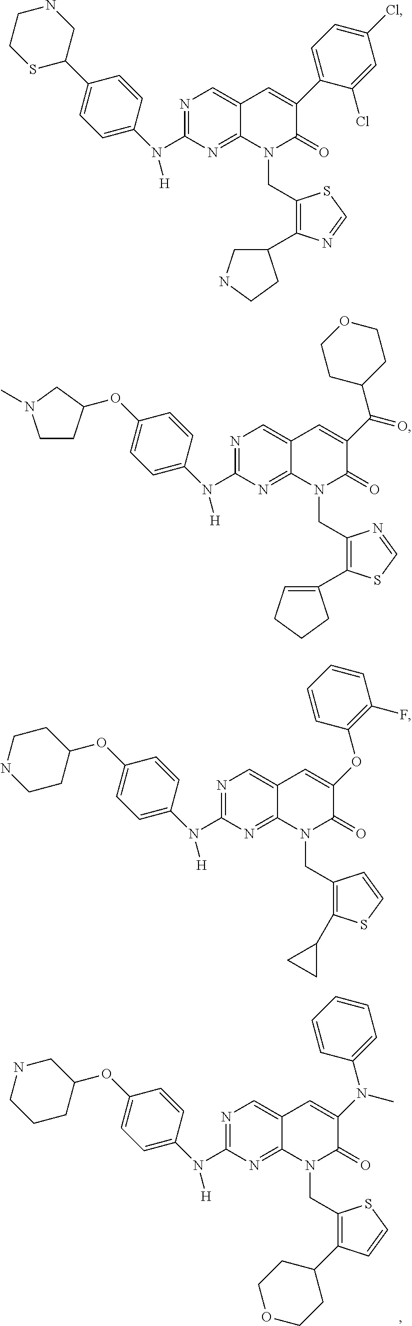

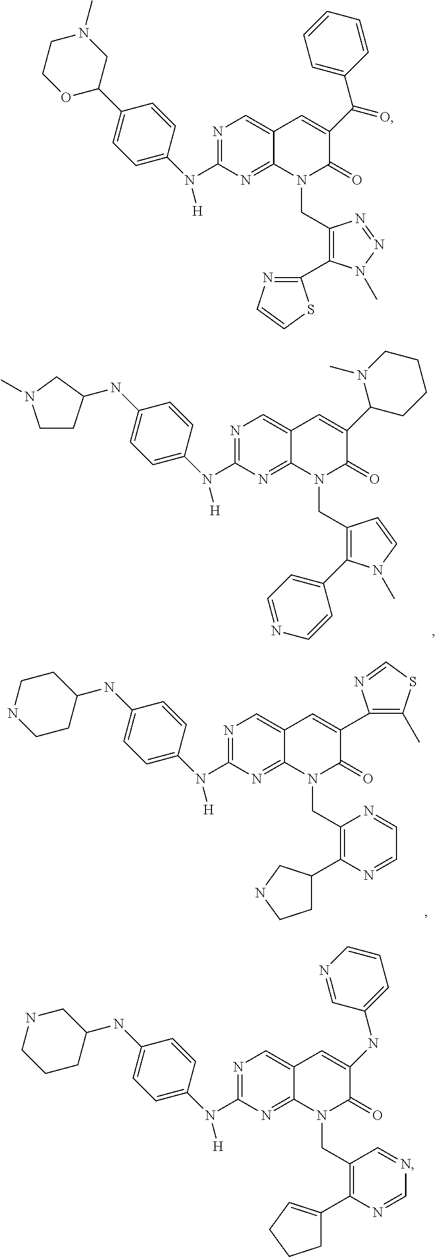 [SMILES: CN1CCOC(c2ccc(Nc3ncc4cc(C(=O)c5ccccc5)c(=O)n(Cc5nnn(C)c5-c5nccs5)c4n3)cc2)C1 ; CN1CCC(Nc2ccc(Nc3ncc4cc(C5CCCCN5C)c(=O)n(Cc5ccn(C)c5-c5ccncc5)c4n3)cc2)C1 ; Cc1scnc1-c1cc2cnc(Nc3ccc(NC4CCNCC4)cc3)nc2n(Cc2nccnc2C2CCNC2)c1=O ; O=c1c(Nc2cccnc2)cc2cnc(Nc3ccc(NC4CCCNC4)cc3)nc2n1Cc1cncnc1C1=CCCC1]

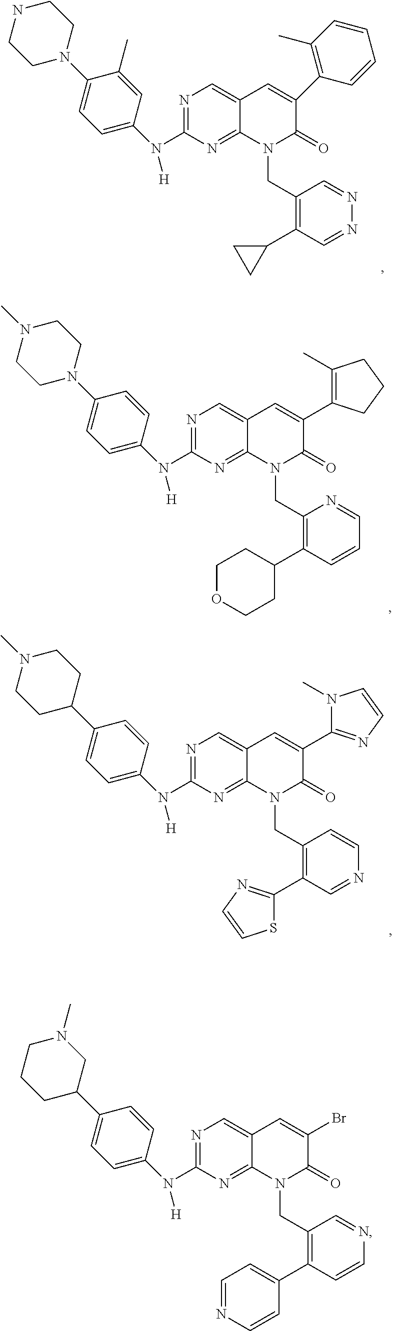 [SMILES: Cc1ccccc1-c1cc2cnc(Nc3ccc(N4CCNCC4)c(C)c3)nc2n(Cc2cnncc2C2CC2)c1=O ; CC1=C(c2cc3cnc(Nc4ccc(N5CCN(C)CC5)cc4)nc3n(Cc3ncccc3C3CCOCC3)c2=O)CCC1 ; CN1CCC(c2ccc(Nc3ncc4cc(-c5nccn5C)c(=O)n(Cc5ccncc5-c5nccs5)c4n3)cc2)CC1 ; CN1CCCC(c2ccc(Nc3ncc4cc(Br)c(=O)n(Cc5cnccc5-c5ccncc5)c4n3)cc2)C1]

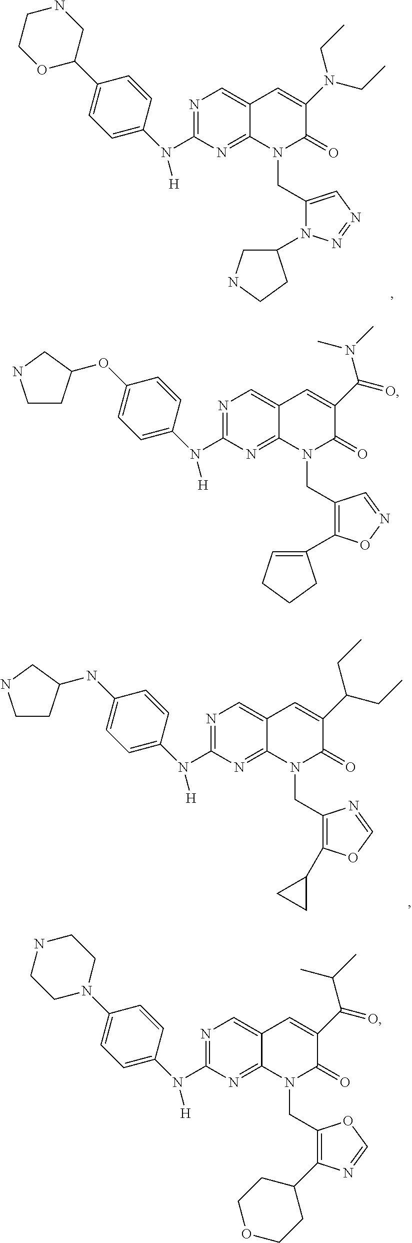 [SMILES: CCN(CC)c1cc2cnc(Nc3ccc(C4CNCCO4)cc3)nc2n(Cc2cnnn2C2CCNC2)c1=O ; CN(C)C(=O)c1cc2cnc(Nc3ccc(OC4CCNC4)cc3)nc2n(Cc2cnoc2C2=CCCC2)c1=O ; CCC(CC)c1cc2cnc(Nc3ccc(NC4CCNC4)cc3)nc2n(Cc2ncoc2C2CC2)c1=O ; CC(C)C(=O)c1cc2cnc(Nc3ccc(N4CCNCC4)cc3)nc2n(Cc2ocnc2C2CCOCC2)c1=O]

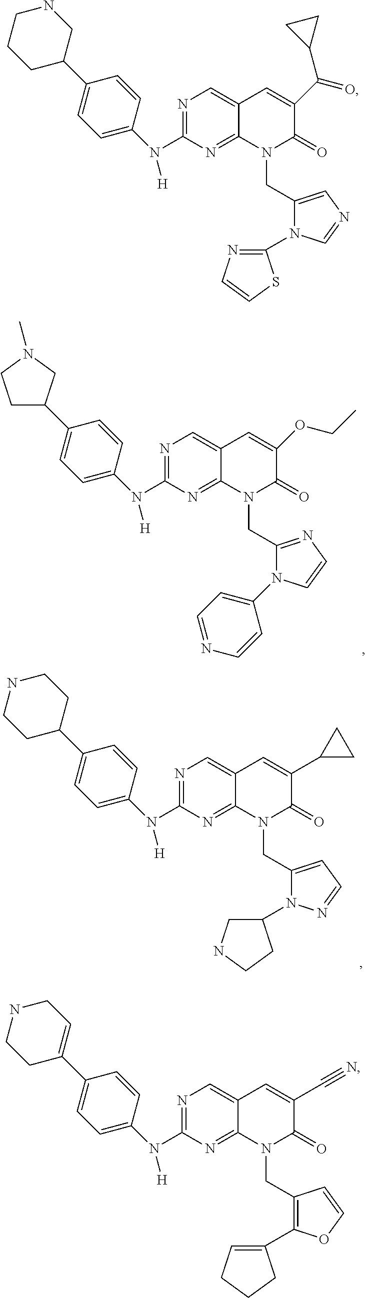 [SMILES: O=C(c1cc2cnc(Nc3ccc(C4CCCNC4)cc3)nc2n(Cc2cncn2-c2nccs2)c1=O)C1CC1 ; CCOc1cc2cnc(Nc3ccc(C4CCN(C)C4)cc3)nc2n(Cc2nccn2-c2ccncc2)c1=O ; O=c1c(C2CC2)cc2cnc(Nc3ccc(C4CCNCC4)cc3)nc2n1Cc1ccnn1C1CCNC1 ; N#Cc1cc2cnc(Nc3ccc(C4=CCNCC4)cc3)nc2n(Cc2ccoc2C2=CCCC2)c1=O]

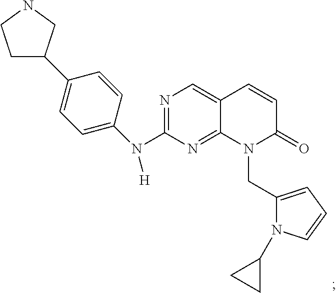 [SMILES: O=c1ccc2cnc(Nc3ccc(C4CCNC4)cc3)nc2n1Cc1cccn1C1CC1]

or a pharmaceutically acceptable salt, solvate or N-oxide thereof.

In another embodiment are PAK inhibitors suitable for treating cancer having the structure:

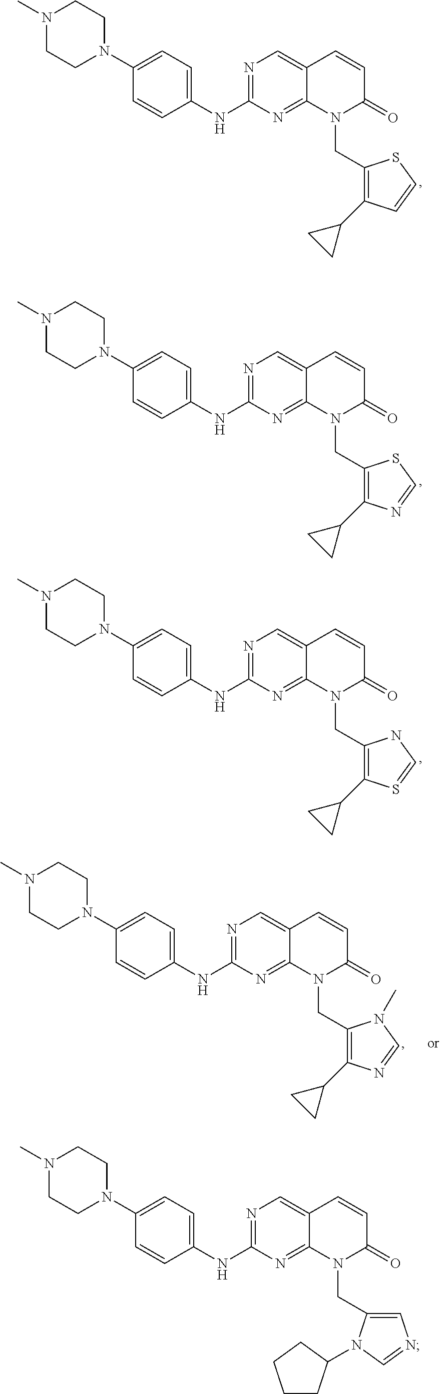

or a pharmaceutically acceptable salt, solvate or N-oxide thereof.

In some embodiments are PAK inhibitors suitable for treating cancer having the structure of Formula II or a pharmaceutically acceptable salt, solvate or N-oxide thereof:

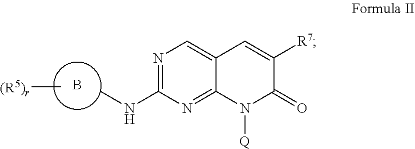

wherein:

R⁷ is

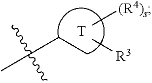

wherein ring T is an aryl, or a heteroaryl ring;
R³ is a substituted or unsubstituted cycloalkyl, a substituted or unsubstituted heteroaryl attached to ring T via a carbon atom of R³, or a substituted or unsubstituted heterocycloalkyl attached to ring T via a carbon atom of R³;
Q is a substituted or unsubstituted alkyl, a substituted or unsubstituted heteroalkyl, a substituted or unsubstituted heterocycloalkyl, a substituted or unsubstituted cycloalkyl, a substituted or unsubstituted cycloalkylalkyl, a substituted or unsubstituted heterocycloalkylalkyl, a substituted or unsubstituted aryl, a substituted or unsubstituted arylalkyl, a substituted or unsubstituted heteroaryl, or a substituted or unsubstituted heteroarylalkyl;
each R⁴ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl;
R⁸ is H or R⁹;
R⁹ is a substituted or unsubstituted alkyl, a substituted or unsubstituted cycloalkyl, a substituted or unsubstituted heterocycloalkyl, a substituted or unsubstituted aryl, or a substituted or unsubstituted heteroaryl;
each R¹⁰ is independently H, a substituted or unsubstituted alkyl, a substituted or unsubstituted cycloalkyl, a substituted or unsubstituted heterocycloalkyl, a substituted or unsubstituted aryl, or a substituted or unsubstituted heteroaryl; or two R¹⁰, together with the atoms to which they are attached form a heterocycle;
ring B is aryl or heteroaryl;
each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl;
r is 0 to 8; and
s is 0 to 4.

In one embodiment is a compound of Formula II wherein ring T is an aryl ring. In one embodiment, the aryl ring is a phenyl group. In another embodiment is a compound of Formula II wherein ring T is a heteroaryl ring. In yet another embodiment is a compound of Formula II, wherein ring T is selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, and pyrazine. In another embodiment, ring T is thiazole.

In a further embodiment is a compound of Formula II, wherein R³ is a C-linked heterocycloalkyl. In one embodiment, the C-linked heterocycloalkyl is oxetane, azetidine, tetrahydrofuran, pyrrolidine, tetrahydrothiophene, piperidine, tetrahydropyran, and morpholine. In a further embodiment, the C-linked heterocycloalkyl is substituted with at least one C₁-C₆alkyl or halogen. In another embodiment, the C₁-C₆alkyl is methyl, ethyl, or n-propyl. In one embodiment is a compound of Formula II, wherein R³ is a substituted or unsubstituted C-linked heteroaryl. In one embodiment, R³ is selected from a C-linked pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, and pyrazine. In yet another embodiment, R³ is a C-linked thiazole. In another embodiment, R³ is a C-linked pyrazole. In a further embodiment, R³ is a C-linked oxadiazole. In another embodiment, R³ is a substituted or unsubstituted cycloalkyl. In a further embodiment, cycloalkyl is selected from cyclopropyl, cyclobutyl, cyclopentyl, or cyclohexyl. In a further embodiment, R³ is cyclopentyl. In another embodiment, R³ is cyclohexyl.

In yet another embodiment, R³ is a C-linked heteroaryl substituted with at least one group selected from halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═)N(R¹⁰)₂, —NR¹⁰C(═O)R¹⁰, —NR¹⁰C(═O)OR¹⁰, —NR¹⁰C(═O)N(R¹⁰)₂, —OR¹⁰, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, the C-linked heteroaryl is substituted with C₁-C₆alkyl. In another embodiment, C₁-C₆alkyl is methyl, ethyl, n-propyl, iso-propyl, n-butyl, iso-butyl, or tert-butyl. In a further embodiment, the C-linked heteroaryl is substituted with methyl. In another embodiment, ethyl. In a further embodiment, n-propyl or iso-propyl.

Also disclosed herein is a compound of Formula II wherein R⁴ is independently halogen, —CN, —NO₂, —OH, —OCF₃, —OCH₂F, —OCF₂H, —CF₃, —SR⁸, —NR¹⁰S(═O)₂R⁹, —S(═O)₂N(R¹⁰)₂, —C(═O)R⁹, —OC(═O)R⁸, —CO₂R¹⁰, —N(R¹⁰)₂, —C(═O)N(R¹⁰)₂, —NR¹⁰C(═O)R¹⁰, —N R¹⁰C(═O)OR¹⁰, and —NR¹⁰C(═O)N(R¹⁰)₂. In a further embodiment, R⁴ is a halogen. In yet another embodiment, R⁴ is selected from F, Cl, Br, or I. In another embodiment, R⁴ is F. In yet another embodiment, R⁴ is a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment, R⁴ is substituted or unsubstituted alkyl selected from methyl, ethyl, n-propyl, iso-propyl, n-butyl, iso-butyl or tert-butyl. In another embodiment, R⁴ is OH. In a further embodiment, R⁴ is OCH₃. In yet another embodiment, R⁴ is OCF₃.

In another embodiment, s is 1. In yet another embodiment, s is 0.

In one embodiment, is a compound of Formula II wherein Q is a substituted or unsubstituted alkyl, a substituted or unsubstituted heteroalkyl, a substituted or unsubstituted heterocycloalkyl, a substituted or unsubstituted cycloalkyl, a substituted or unsubstituted cycloalkylalkyl, a substituted or unsubstituted heterocycloalkylalkyl, a substituted or unsubstituted aryl, a substituted or unsubstituted arylalkyl, a substituted or unsubstituted heteroaryl, or a substituted or unsubstituted heteroarylalkyl. In another embodiment, Q is a substituted or unsubstituted alkyl. In a further embodiment, Q is an unsubstituted methyl, ethyl, n-propyl, iso-propyl, n-butyl, iso-butyl or tert-butyl. In a further embodiment, Q is ethyl.

In yet another embodiment, is a compound of Formula II, wherein ring B is an aryl ring. In another embodiment, ring B is a substituted or unsubstituted phenyl. In a further embodiment, ring B is a substituted or unsubstituted naphthalene. In a further embodiment, is a compound of Formula II, wherein ring B is a heteroaryl ring selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, and pyrazine.

In yet a further embodiment, is a compound of Formula II, wherein R⁵ is a C₃-C₆ cycloalkyl ring; or a 3-6-membered heterocycloalkyl ring comprising 1-3 N atoms, an O atom, a S atom; or any combination thereof, and wherein R⁵ is further substituted by halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═)R⁹, —CO₂R¹⁰, —N(R¹⁰)₂, —C(═O)N(R¹⁰)₂, —NR¹⁰C(═O)R¹⁰, —NR¹⁰C(═)OR¹⁰, —NR¹⁰C(═O)N(R¹⁰)₂, —OR¹⁰, substituted or unsubstituted alkyl, substituted or unsubstituted alkoxy, substituted or unsubstituted heteroalkyl, substituted or unsubstituted cycloalkyl or substituted or unsubstituted heterocycloalkyl.

In another embodiment, is a compound of Formula II wherein R⁵ is a C₃-C₆cycloalkyl ring. In another embodiment, R⁵ is cyclopropyl. In another embodiment, R⁵ is cyclopentyl. In another embodiment, R⁵ is cyclohexyl.

In another embodiment, is a compound of Formula II wherein R⁵ is a substituted or unsubstituted heterocycloalkyl ring. In another embodiment, R⁵ is a substituted or unsubstituted piperazine. In another embodiment, R⁵ is an unsubstituted piperazine. In another embodiment, the heterocycloalkyl ring is N-methylpiperazine. In another embodiment, R⁵ is a substituted or unsubstituted piperidine. In another embodiment, R⁵ is an unsubstituted piperidine.

In another embodiment, R⁵ is OH or CN. In a further embodiment, R⁵ is OCF₃, or CF₃.

In another embodiment is a compound of Formula II wherein r is 0. In another embodiment, r is 1. In a further embodiment, r is 2. In another embodiment is a compound of Formula II wherein r is 1; and R⁵ is substituted or unsubstituted piperazine.

In one embodiment is a compound of Formula II wherein

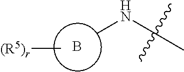

is selected from:

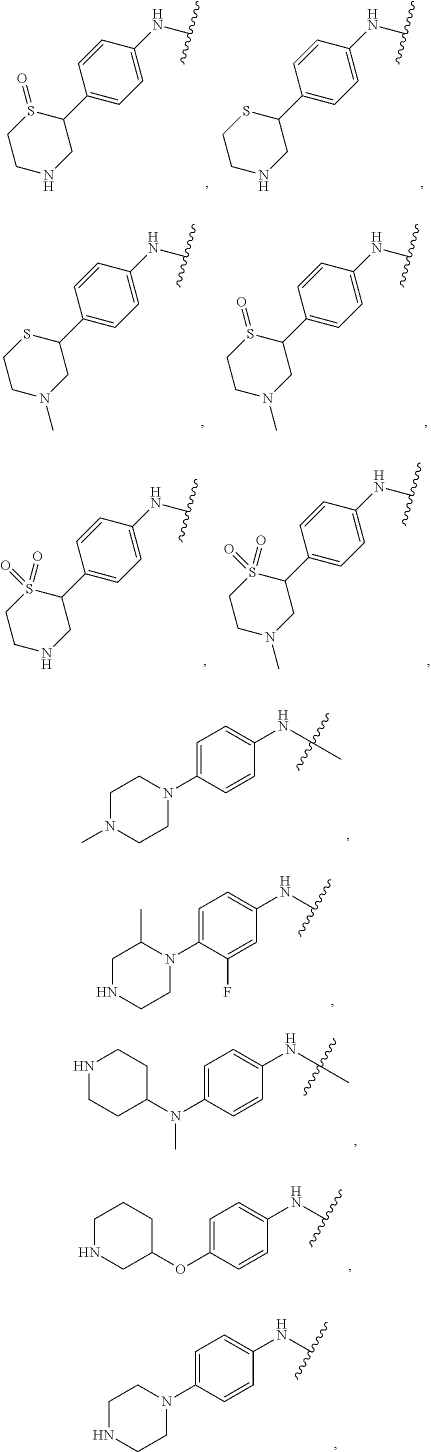

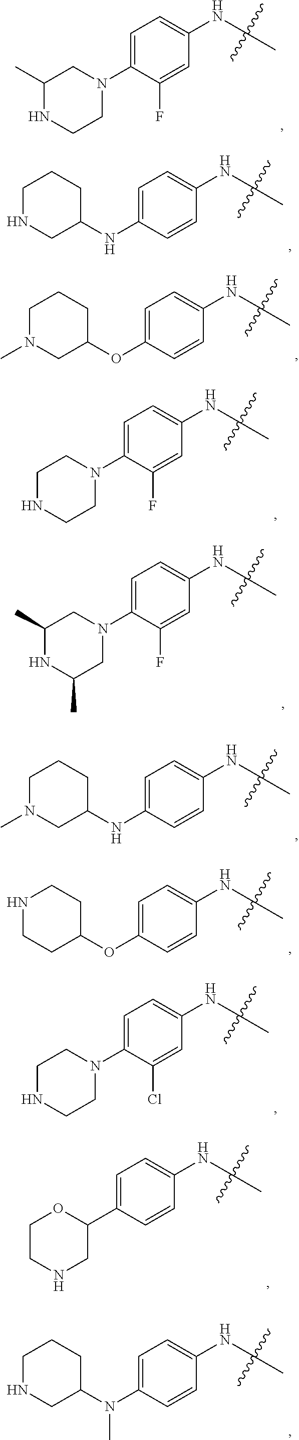

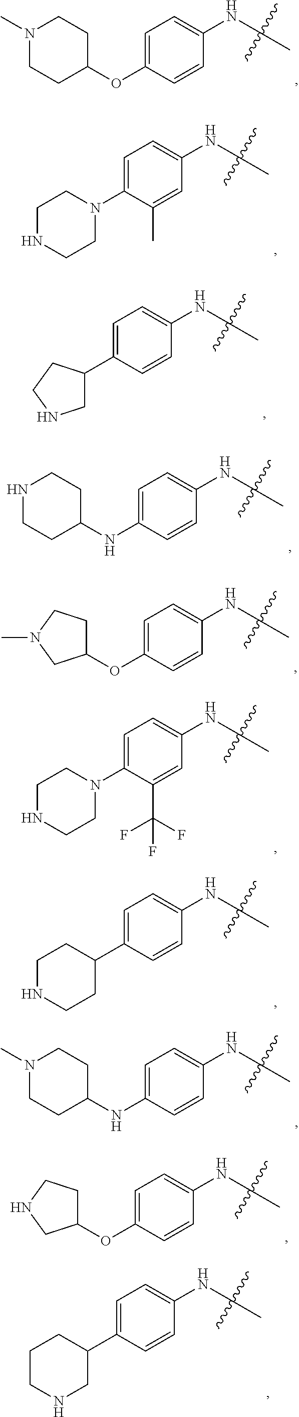

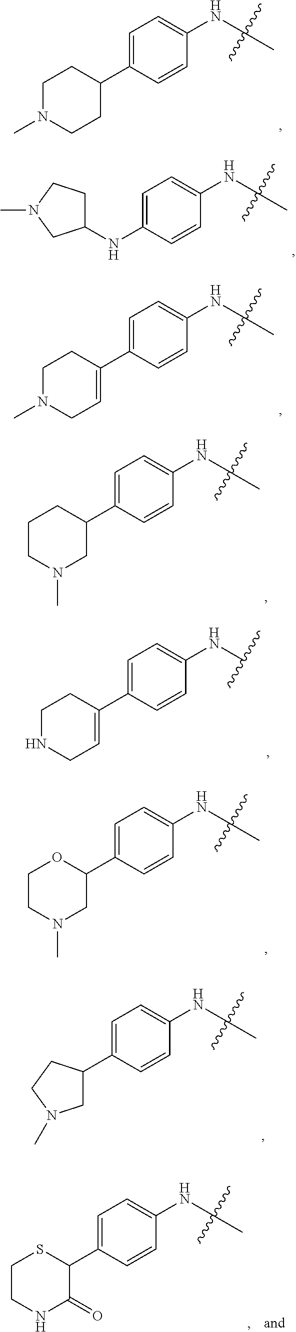

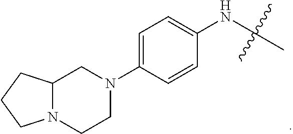 [SMILES: *Nc1ccc(N2CCN3CCCC3C2)cc1]

In one embodiment, is a compound of Formula II, wherein R⁵ is halogen, —CN, —OH, substituted or unsubstituted alkyl, —OR¹⁰, —NR¹⁰S(═O)₂R⁹, —S(═)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In one embodiment, R⁵ is selected from F, Cl, Br, or I. In another embodiment R⁵ is F.

In another embodiment, is a compound of Formula II, wherein at least one R⁵ is —NR¹⁰S(═O)₂R⁹, —S(═O)₂N(R¹⁰)₂, —N(R¹⁰)₂, —C(═)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In one embodiment, is a compound of Formula II, wherein at least one R⁵ is —N(R¹⁰)₂, or substituted or unsubstituted heterocycloalkyl. In yet another embodiment, is a compound of Formula II wherein at least one of R⁵ is a substituted or unsubstituted piperazine, substituted or unsubstituted piperidine, substituted or unsubstituted pyrrolidine or substituted or unsubstituted morpholine. In a further embodiment, is a compound of Formula II, wherein at least one R⁵ is —OR¹⁰. In one embodiment is a compound of Formula II, wherein at least one R⁵ is —OR¹⁰ and R¹⁰ is H. In another embodiment, R¹⁰ is alkyl selected from methyl, ethyl, n-propyl, iso-propyl, n-butyl, iso-butyl, and tert-butyl.

In one embodiment is a compound of Formula II wherein ring B is substituted with —N(R¹⁰)₂, wherein R¹⁰ is each independently selected from H and a substituted or unsubstituted heterocycloalkyl. In another embodiment is a compound of Formula II wherein ring B is substituted with —NHR¹⁰ wherein R¹⁰ is a substituted or unsubstituted piperazine, substituted or unsubstituted piperidine, substituted or unsubstituted pyrrolidine or substituted or unsubstituted morpholine. In a further embodiment is a compound of Formula II wherein ring B is substituted with —N(CH₃)R¹⁰ wherein R¹⁰ is a substituted or unsubstituted piperazine, substituted or unsubstituted piperidine, substituted or unsubstituted pyrrolidine or substituted or unsubstituted morpholine.

Also presented herein is a compound of Formula II wherein ring B is substituted with —OR¹⁰ wherein R¹⁰ is a substituted or unsubstituted heterocycloalkyl. In another embodiment is a compound of Formula II wherein ring B is substituted with —OR¹⁰ wherein R¹⁰ is a substituted or unsubstituted piperazine, substituted or unsubstituted piperidine, substituted or unsubstituted pyrrolidine or substituted or unsubstituted morpholine. In yet another embodiment is a compound of Formula II wherein ring B is substituted with at least one CF₃.

In yet another embodiment, ring B is substituted with at least two R⁵. In another embodiment, ring B is substituted with halogen and a substituted or unsubstituted heterocycloalkyl. In another embodiment, ring B is substituted with at least one F, Cl, Br, or I and a substituted or unsubstituted piperazine, substituted or unsubstituted piperidine, substituted or unsubstituted pyrrolidine, or substituted or unsubstituted morpholine.

In some embodiments are PAK inhibitors suitable for treating cancer having the structure of Formula IIa or a pharmaceutically acceptable salt, solvate or N-oxide thereof:

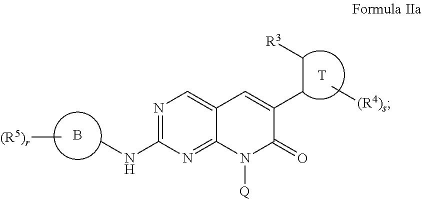

wherein:
ring T is an aryl, or a heteroaryl ring;
R³ is a substituted or unsubstituted cycloalkyl, a substituted or unsubstituted heteroaryl attached to ring T via a carbon atom of R³, or a substituted or unsubstituted heterocycloalkyl attached to ring T via a carbon atom of R³;
each R⁴ is independently halogen, —CN, —NO₂, —OH, —OCF₃, —OCF₂H, —CF₃, —S(═O)R⁹, —S(═O)₂R⁹, —NR¹⁰S(═O)₂R⁹, —S(═O)₂N(R¹⁰)₂, —OR¹⁰, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted heteroalkyl, substituted or unsubstituted cycloalkyl or substituted or unsubstituted heterocycloalkyl;
R⁸ is H or R⁹;
R⁹ is a substituted or unsubstituted alkyl, a substituted or unsubstituted cycloalkyl, a substituted or unsubstituted heterocycloalkyl, a substituted or unsubstituted aryl, or a substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl or substituted or unsubstituted heteroaryl, or two R¹⁰ together with the atoms to which they are attached form a heterocycle;
s 0-4;
ring B is aryl or heteroaryl;
each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, substituted or unsubstituted alkyl, substituted or unsubstituted heteroalkyl, substituted or unsubstituted cycloalkyl or substituted or unsubstituted heterocycloalkyl; and
r is 0-8.

In a further embodiment are PAK inhibitors suitable for treating cancer having the structure of Formula IIb or a pharmaceutically acceptable salt, solvate or N-oxide thereof:

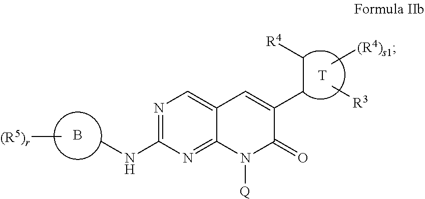

wherein s1 is 0 to 3 and ring T, ring B, R³, R⁴, R⁵, Q and r are described previously.

In yet a further embodiment are PAK inhibitors suitable for treating cancer having the structure of Formula IIc or a pharmaceutically acceptable salt, solvate or N-oxide thereof:

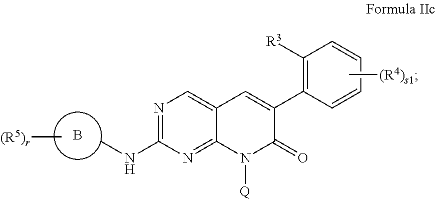

wherein s1 is 0 to 4 and ring B, R³, R⁴, R⁵, Q and r are described previously.

In another embodiment are PAK inhibitors suitable for treating cancer having the structure of Formula IId or a pharmaceutically acceptable salt, solvate or N-oxide thereof:

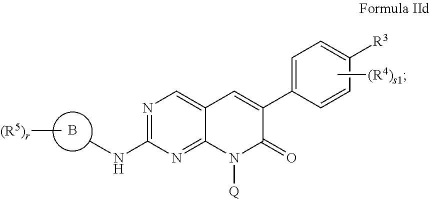

wherein s1 is 0 to 4 and ring B, R³, R⁴, R⁵, Q and r are described previously.

In another embodiment are PAK inhibitors suitable for treating cancer having the structure of Formula IIe or a pharmaceutically acceptable salt, solvate or N-oxide thereof:

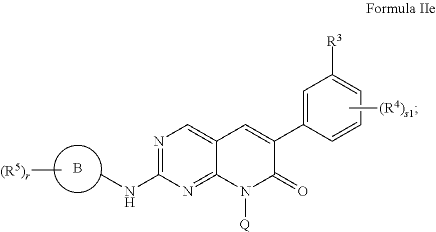

wherein s1 is 0 to 4 and ring B, R³, R⁴, R⁵, Q and r are described previously.

In another embodiment are PAK inhibitors suitable for treating cancer having the structure of Formula IIf or a pharmaceutically acceptable salt, solvate or N-oxide thereof:

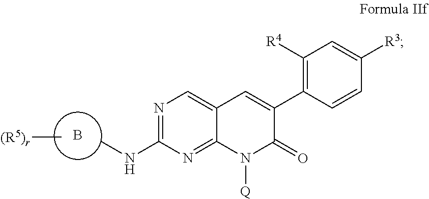

wherein ring B, R³, R⁴, R⁵, Q and r are described previously.

In one embodiment is a compound of Formula II, IIa, IIb, IIc, IId, IIe, or IIf wherein R³ is selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, and pyrazine.

In another embodiment, is a compound of Formula II, IIa, IIb, IIc, IId, IIe, or IIf wherein R³ is selected from

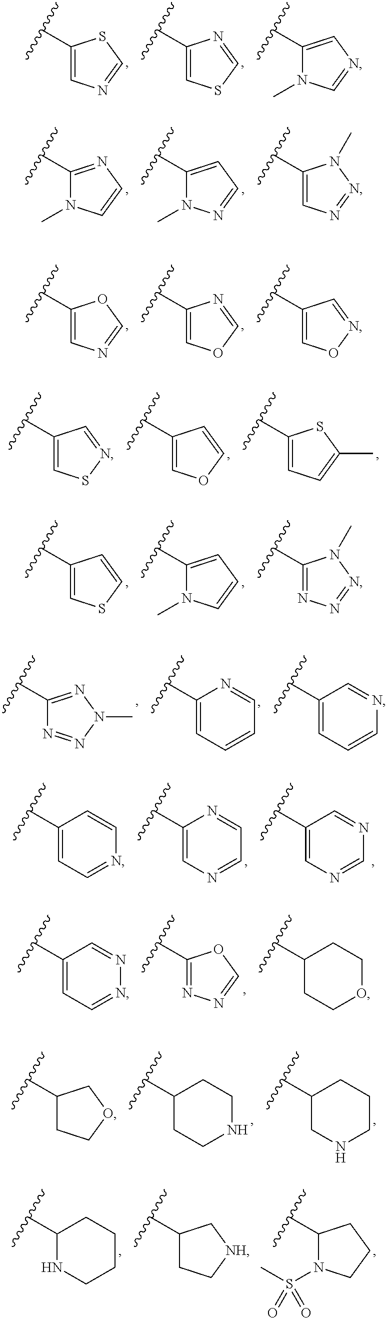

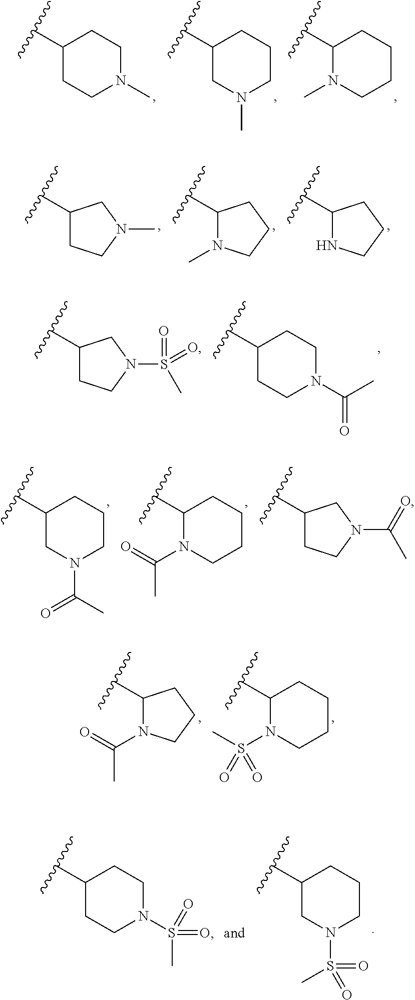

In a further embodiment is a compound of Formula II, IIa, IIb, IIc, IId, IIe, or IIf; wherein

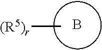

is:

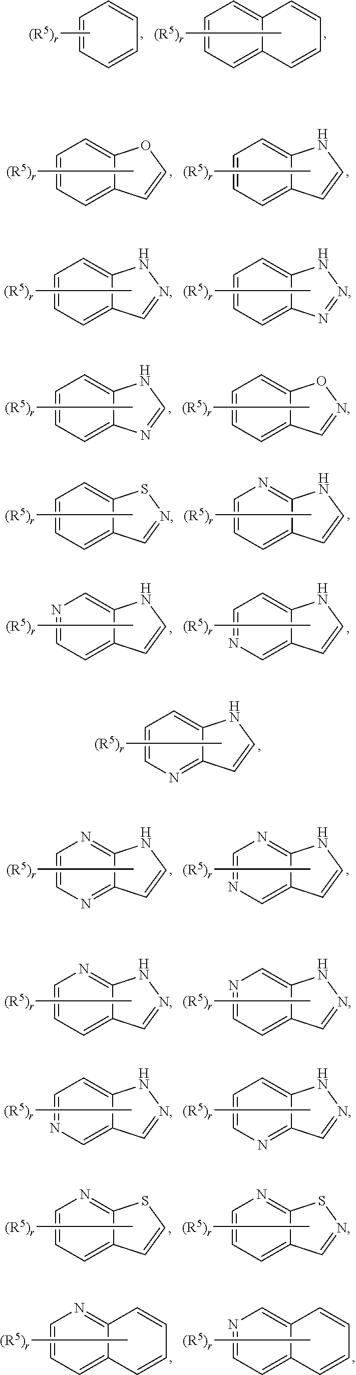

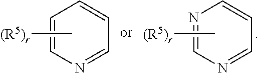

In another embodiment is a compound of Formula II, IIa, IIb, IIc, IId, IIe, or IIf, where R⁵ is halogen, —CN, —OH, a substituted or unsubstituted alkyl, —OR¹⁰, —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or a substituted or unsubstituted heterocycloalkyl.

In one embodiment is a compound of Formula II, IIa, IIb, IIc, IId, IIe, or IIf, wherein at least one R⁵ is —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or a substituted or unsubstituted heterocycloalkyl.

In one embodiment is a compound of Formula II, IIa, IIb, IIc, IId, IIe, or IIf, wherein at least one R⁵ is —N(R¹⁰)₂, or a substituted or unsubstituted heterocycloalkyl. In a further embodiment is a compound of Formula II, IIa, IIb, IIc, IId, IIe, or IIf wherein at least one of R⁵ is a substituted or unsubstituted piperazine, a substituted or unsubstituted piperidine, a substituted or unsubstituted pyrrolidine, or a substituted or unsubstituted morpholine. In one embodiment is a compound of Formula II, IIa, IIb, IIc, IId, IIe, or IIf, wherein at least one R⁵ is —OR¹⁰. In another embodiment is a compound of Formula I, II, III, IV, V, Va, or Vb, wherein R⁴ is independently halogen, —CN, —OH, —OCF₃, —OCF₃, —OCF₂H, —CF₃, —SR⁸, a substituted or unsubstituted alkyl, or a substituted or unsubstituted alkoxy.

In one embodiment is a compound of Formula II, IIa, IIb, IIc, IId, IIe, or IIf, wherein s is zero.

In a further embodiment is a compound of Formula II, IIa, IIb, IIc, IId, IIe, or IIf; wherein Q is a substituted or unsubstituted alkyl, or a substituted or unsubstituted heteroalkyl. In another embodiment is a compound of Formula II, IIa, IIb, IIc, IId, IIe, or IIf; wherein Q is a substituted or unsubstituted cycloalkyl, or a substituted or unsubstituted heterocycloalkyl. In a further embodiment is a compound of Formula II, IIa, IIb, IIc, IId, IIe, or IIf; wherein Q is a substituted or unsubstituted cycloalkylalkyl, or a substituted or unsubstituted heterocycloalkylalkyl. In one embodiment is a compound of Formula II, IIa, IIb, IIc, IId, IIe, or IIf, wherein Q is a substituted or unsubstituted aryl, or a substituted or unsubstituted heteroaryl.

In one embodiment is a compound of Formula II, IIa, IIb, IIc, IId, IIe, or IIf, wherein Q is a substituted or unsubstituted arylalkyl, or a substituted or unsubstituted heteroarylalkyl.

In another embodiment is a compound of Formula II, IIa, IIb, IIc, IId, IIe, or IIf, wherein Q is selected from:

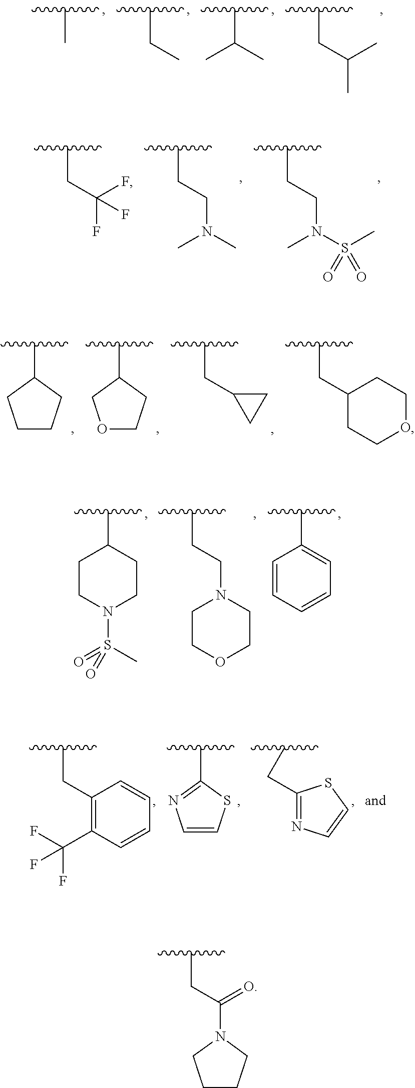

Also provided herein, in some embodiments, are PAK inhibitors suitable for treating cancer having the structure of Formula IIg or a pharmaceutically acceptable salt, solvate or N-oxide thereof:

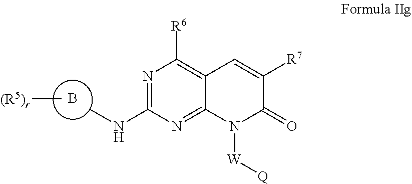

wherein:
W is a bond;
R⁶ is —CN, —OH, substituted or unsubstituted alkoxy, —N(R¹⁰)₂, substituted or unsubstituted heteroalkyl, substituted or unsubstituted cycloalkyl, substituted or unsubstituted aryl or substituted or unsubstituted heteroaryl;
R⁷ is halogen, —CN, —OH, substituted or unsubstituted alkoxy, —C(═O)N(R¹⁰)₂, —CO₂R¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl, or substituted or unsubstituted cycloalkyl or heterocycloalkyl fused to ring A;
ring A is substituted or unsubstituted aryl or heteroaryl substituted with 0-4 R⁴;
   each R⁴ is independently halogen, —CN, —NO₂, —OH, —S(═O)R⁹, —S(═O)₂R⁹, —NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —S(═O)R⁹, —S(═O)₂R⁹, NR^{10s}(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8.

In one embodiment are PAK inhibitors suitable for treating cancer having the structure of Formula IIg or a pharmaceutically acceptable salt, solvate or N-oxide thereof wherein:
W is a bond;
R⁶ is —CN, —OH, substituted or unsubstituted alkoxy, —N(R¹⁰)₂, substituted or unsubstituted heteroalkyl, substituted or unsubstituted cycloalkyl, substituted or unsubstituted aryl or substituted or unsubstituted heteroaryl;
R⁷ is halogen, —CN, —OH, substituted or unsubstituted alkoxy, —C(═O)N(R¹⁰)₂, —CO₂R¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
Q substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl;
ring A is substituted or unsubstituted aryl or heteroaryl substituted with 0-4 R⁴; each R⁴ is independently halogen, —CN, —NO₂, —OH, —S(═O)R⁹, —S(═O)₂R⁹, —NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
   R⁸ is H or substituted or unsubstituted alkyl;
   R⁹ is substituted or unsubstituted alkyl, substituted or unsubstituted cycloalkyl, substituted or unsubstituted aryl or substituted or unsubstituted heteroaryl
   each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8.

In another embodiment are PAK inhibitors suitable for treating cancer having the structure of Formula IIg or a pharmaceutically acceptable salt, solvate or N-oxide thereof wherein:
W is a bond;
R⁶ is —CN, —OH, substituted or unsubstituted alkoxy, —N(R¹⁰)₂, substituted or unsubstituted heteroalkyl, substituted or unsubstituted cycloalkyl, substituted or unsubstituted aryl or substituted or unsubstituted heteroaryl;
R⁷ is halogen, —CN, —OH, substituted or unsubstituted alkoxy, —C(═O)N(R¹⁰)₂, —CO₂R¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
Q is an unsubstituted alkyl;
ring A is substituted or unsubstituted aryl or heteroaryl substituted with 0-4 R⁴;
   each R⁴ is independently halogen, —CN, —NO₂, —OH, —S(═O)R⁹, —S(═O)₂R⁹, —NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8.

In yet another embodiment are PAK inhibitors suitable for treating cancer having the structure of Formula IIg or a pharmaceutically acceptable salt, solvate or N-oxide thereof wherein:
W is a bond;
R⁶ is —CN, —OH, substituted or unsubstituted alkoxy, —N(R¹⁰)₂, substituted or unsubstituted heteroalkyl, substituted or unsubstituted cycloalkyl, substituted or unsubstituted aryl or substituted or unsubstituted heteroaryl;
R⁷ is halogen, —CN, —OH, substituted or unsubstituted alkoxy, —C(═O)N(R¹⁰)₂, —CO₂R¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
Q is a substituted alkyl;
ring A is substituted or unsubstituted aryl or heteroaryl substituted with 0-4 R⁴;
   each R⁴ is independently halogen, —CN, —NO₂, —OH, —SR⁸, —S(═O)R⁹, —S(═O)₂R⁹, —NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      r is 0-8.

Provided herein, in some embodiments, are PAK inhibitors suitable for treating cancer having the structure of Formula IIh or a pharmaceutically acceptable salt, solvate or N-oxide thereof wherein:

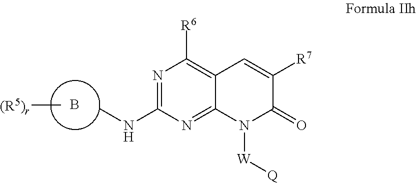

wherein:
W is a bond;
R⁶ is substituted or unsubstituted heteroalkyl, substituted or unsubstituted cycloalkyl, substituted or unsubstituted aryl or substituted or unsubstituted heteroaryl;
R⁷ is H, halogen, —CN, —OH, substituted or unsubstituted alkyl, substituted or unsubstituted alkoxy, —C(═O)N(R¹⁰)₂, —CO₂R¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl;
ring A is substituted or unsubstituted aryl or heteroaryl substituted with 0-4 R⁴;
   each R⁴ is independently halogen, —CN, —NO₂, —OH, —S(═O)R⁹, —S(═O)₂R⁹, —NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
   r is 0-8.

In one embodiment is a compound of Formula IIh wherein Q is substituted or unsubstituted alkyl. In a further embodiment is a compound of Formula IIh wherein Q is a substituted alkyl. In yet another embodiment is a compound of Formula IIh wherein Q is an unsubstituted alkyl. In a further embodiment is a compound of Formula IIh wherein Q is substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl.

Provided herein, in some embodiments, are PAK inhibitors suitable for treating cancer having the structure of Formula IIi or a pharmaceutically acceptable salt, solvate or N-oxide thereof wherein:

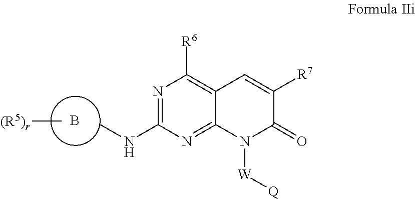

wherein:
W is a bond;
R⁶ is H, or halogen;
R⁷ is acyl, substituted or unsubstituted heteroalkyl, substituted or unsubstituted cycloalkyl, substituted or unsubstituted heterocycloalkyl or substituted or unsubstituted heteroaryl;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl;
ring A is substituted or unsubstituted aryl or heteroaryl substituted with 0-4 R⁴;
   each R⁴ is independently halogen, —CN, —NO₂, —OH, —S(═O)R⁹, —S(═O)₂R⁹, —NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁸, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
   r is 0-8.

In one embodiment is a compound of Formula III wherein Q is substituted or unsubstituted alkyl. In a further embodiment is a compound of Formula III wherein Q is a substituted alkyl. In yet another embodiment is a compound of Formula III wherein Q is an unsubstituted alkyl. In a further embodiment is a compound of Formula III wherein Q is substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl.

Also provided herein, in some embodiments, are PAK inhibitors suitable for treating cancer having the structure of Formula IIj or a pharmaceutically acceptable salt, solvate or N-oxide thereof wherein:

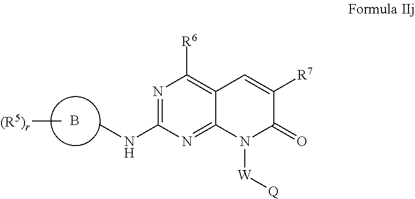

wherein:
W is a bond;
R⁶ is substituted or unsubstituted alkyl;
R⁷ is substituted or unsubstituted heteroalkyl, substituted or unsubstituted cycloalkyl, or substituted or unsubstituted heterocycloalkyl;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl;
ring A is substituted or unsubstituted aryl or heteroaryl substituted with 0-4 R⁴;
   each R⁴ is independently halogen, —CN, —NO₂, —OH, —S(═O)R⁹, —S(═O)₂R⁹, —NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
ring B is aryl or heteroaryl substituted with R⁵;
   each R⁵ is independently halogen, —CN, —NO₂, —OH, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰,
      —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      r is 0-8.

In one embodiment is a compound of Formula IIj wherein Q is substituted or unsubstituted alkyl. In a further embodiment is a compound of Formula IIj wherein Q is a substituted alkyl. In yet another embodiment is a compound of Formula IIj wherein Q is an unsubstituted alkyl. In a further embodiment is a compound of Formula IIj wherein Q is substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl.

Provided herein, in some embodiments, are PAK inhibitors suitable for treating cancer having the structure of Formula IIk or a pharmaceutically acceptable salt, solvate or N-oxide thereof wherein:

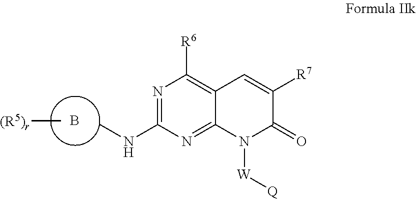

wherein:
W is a bond;
R⁶ is H;
R⁷ is

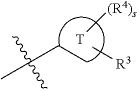

ring T is aryl, heteroaryl, cycloalkyl or heterocycloalkyl substituted with R³ and R⁴;
R³ is a substituted or unsubstituted aryl, substituted or unsubstituted heteroaryl, substituted or unsubstituted cycloalkyl or substituted or unsubstituted heterocycloalkyl attached to ring T via a carbon atom;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl;
ring A is substituted or unsubstituted aryl or heteroaryl substituted with 0-4 R⁴;
   each R⁴ is independently halogen, —CN, —NO₂, —OH, —S(═O)R⁹, —S(═O)₂R⁹, —NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      R⁸ is H or substituted or unsubstituted alkyl;
      R⁹ is substituted or unsubstituted alkyl, substituted or unsubstituted cycloalkyl, substituted or unsubstituted aryl or substituted or unsubstituted heteroaryl
      each R¹⁰ is independently H, substituted or unsubstituted alkyl, substituted or unsubstituted cycloalkyl, substituted or unsubstituted aryl or substituted or unsubstituted heteroaryl, or two R¹⁰ together with the atoms to which they are attached form a heterocycle;
   s is 0-4;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
      r is 0-8.

In one embodiment is a compound of Formula IIk wherein Q is substituted or unsubstituted alkyl. In a further embodiment is a compound of Formula IIk wherein Q is a substituted alkyl. In yet another embodiment is a compound of Formula IIk wherein Q is an unsubstituted alkyl. In a further embodiment is a compound of Formula IIk wherein Q is substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl.

Provided herein, in some embodiments, are PAK inhibitors suitable for treating cancer having the structure of Formula IIm or a pharmaceutically acceptable salt, solvate or N-oxide thereof wherein:

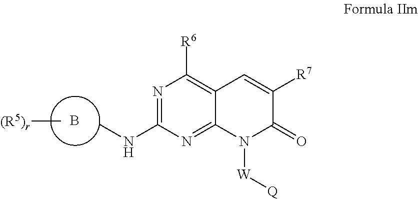

wherein:
W is N—R^{1a};
R^{1a} is H or substituted or unsubstituted alkyl;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, or substituted or unsubstituted heteroarylalkyl;
ring B is aryl or heteroaryl substituted with R⁵;
   each R⁵ is independently halogen, —CN, —NO₂, —OH, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8;
R⁶ is H, halogen, —CN, —OH, substituted or unsubstituted alkyl, substituted or unsubstituted alkoxy, substituted or unsubstituted heteroalkyl, —N(R¹⁰)₂, substituted or unsubstituted cycloalkyl, substituted or unsubstituted aryl or substituted or unsubstituted heteroaryl;
R⁷ is H, halogen, —CN, —OH, acyl, substituted or unsubstituted alkyl, substituted or unsubstituted alkoxy, —C(═O)N(R¹⁰)₂, —CO₂R¹⁰, —N(R¹⁰)₂, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In one embodiment is a compound of Formula IIm wherein Q is substituted or unsubstituted alkyl. In a further embodiment is a compound of Formula IIm wherein Q is a substituted alkyl. In yet another embodiment is a compound of Formula IIm wherein Q is an unsubstituted alkyl. In a further embodiment is a compound of Formula IIm wherein Q is substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, substituted or unsubstituted heteroarylalkyl.

In another embodiment are PAK inhibitors suitable for treating cancer having the structure of Formula IIn or a pharmaceutically acceptable salt, solvate or N-oxide thereof wherein:

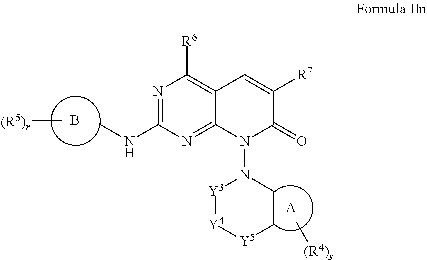

wherein:
each of Y³, Y⁴ and Y⁵ are independently N—R^{1a}, CR¹R², SO₂, or C═O;
R¹ and R² are each independently H or substituted or unsubstituted alkyl;
R^{1a} is H or substituted or unsubstituted alkyl;
Q is substituted or unsubstituted alkyl, substituted or unsubstituted heteroalkyl, substituted or unsubstituted heterocycloalkyl, substituted or unsubstituted cycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, or substituted or unsubstituted heteroarylalkyl;
ring A is aryl or heteroaryl;
ring B is aryl or heteroaryl substituted with R⁵;
   each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8;
s is 0-4;
R⁴ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁸, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl;
R⁶ is H, halogen, —CN, —OH, substituted or unsubstituted alkyl, substituted or unsubstituted alkoxy, substituted or unsubstituted heteroalkyl, —N(R¹⁰)₂, substituted or unsubstituted cycloalkyl, substituted or unsubstituted aryl or substituted or unsubstituted heteroaryl;
R⁷ is H, halogen, —CN, —OH, acyl, substituted or unsubstituted alkyl, substituted or unsubstituted alkoxy, —C(═O)N(R¹⁰)₂, —CO₂R¹⁰, —N(R¹⁰)₂, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In some embodiments, a compound of Formula IIm has the structure of Formula IIo:

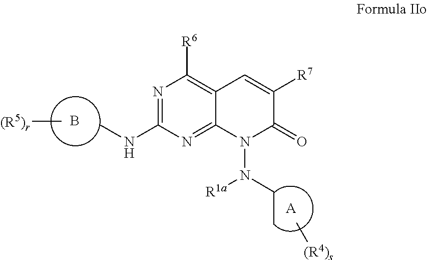

In some embodiments, a compound of Formula IIm has the structure of Formula IIp:

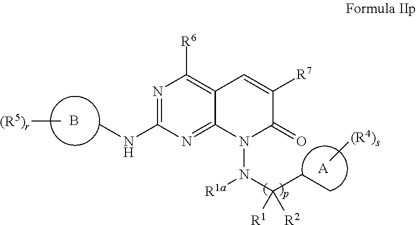

wherein:
p is 1, 2 or 3;
   R¹ and R² are each independently H or substituted or unsubstituted alkyl; or R¹ and R² together
with the carbon to which they are attached form a C₃-C₆ cycloalkyl ring.

In some embodiments of Formula IIp, ring A is a heteroaryl ring. In some embodiments of Formula IIp, ring A is an aryl ring. In some embodiments of Formula IIp, ring A is a heterocycloalkyl ring. In some embodiments of Formula IIp, ring A is a cycloalkyl ring.

In some embodiments, the compound of Formula IIm has the structure of Formula IIq:

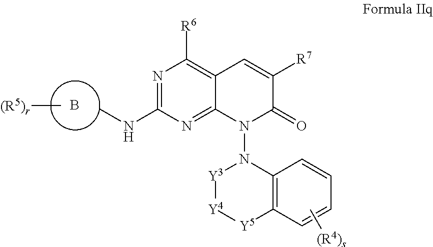

wherein:
each of Y³, Y⁴ and Y⁵ are independently N—R^{1a}, CR¹R², SO₂, or C═O;
R^{1a} is H or substituted or unsubstituted alkyl;
   R¹ and R² are each independently H or substituted or unsubstituted alkyl.

In some embodiments, the compound of Formula IIm has the structure of Formula IIr, Formula IIs, Formula IIt or Formula IIu:

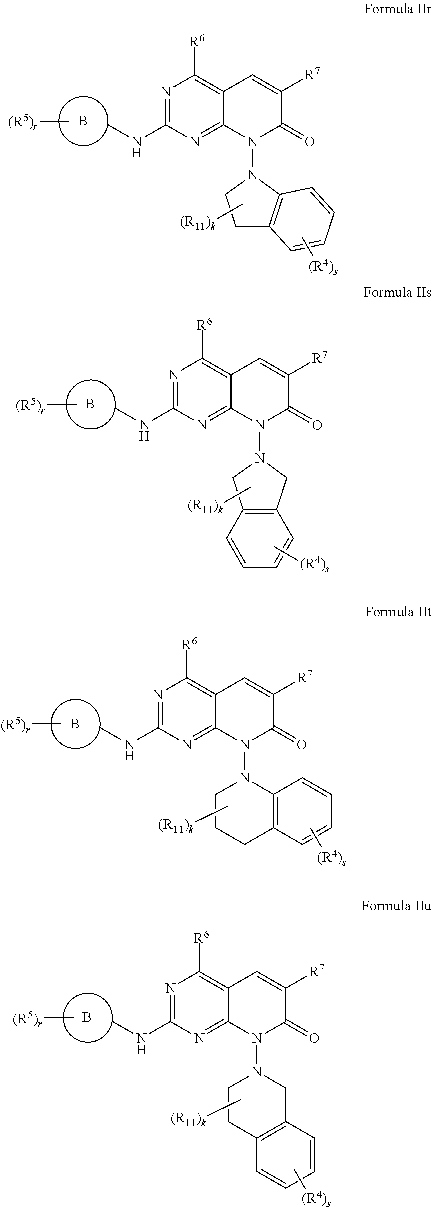

wherein:
each R¹¹ is independently H, halogen, substituted or unsubstituted alkyl, substituted or unsubstituted alkoxy, or two R¹¹ together with the carbon atom to which they are attached form C═O; and
k is 1-4.

In a further embodiment is a compound having the structure:

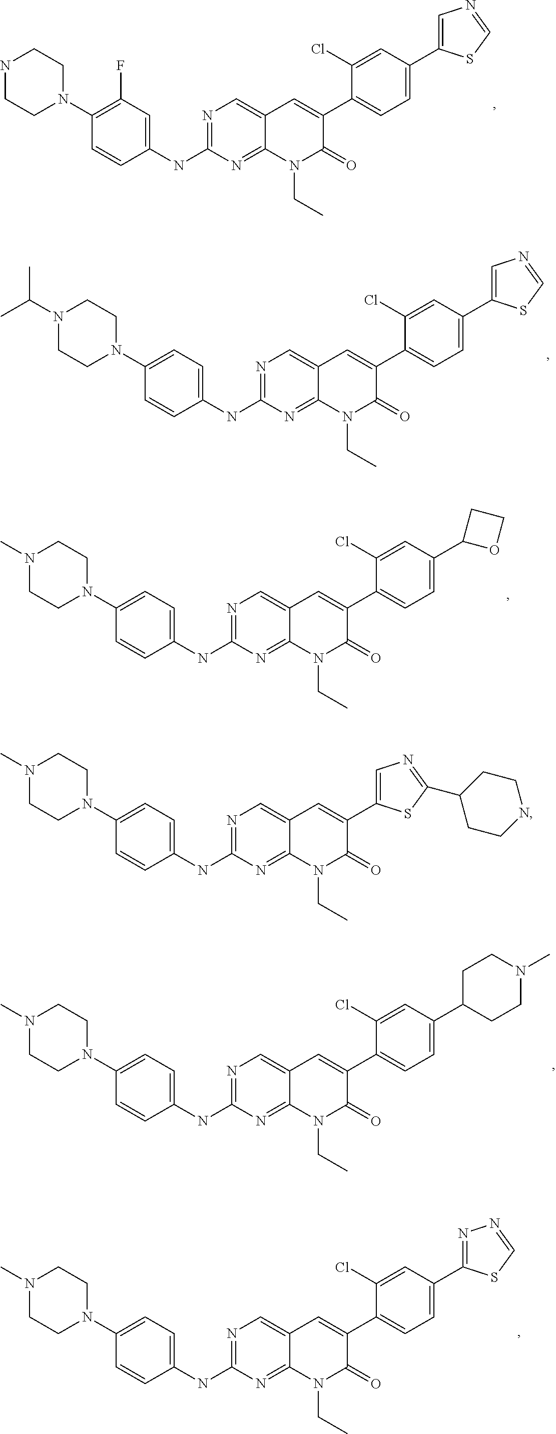

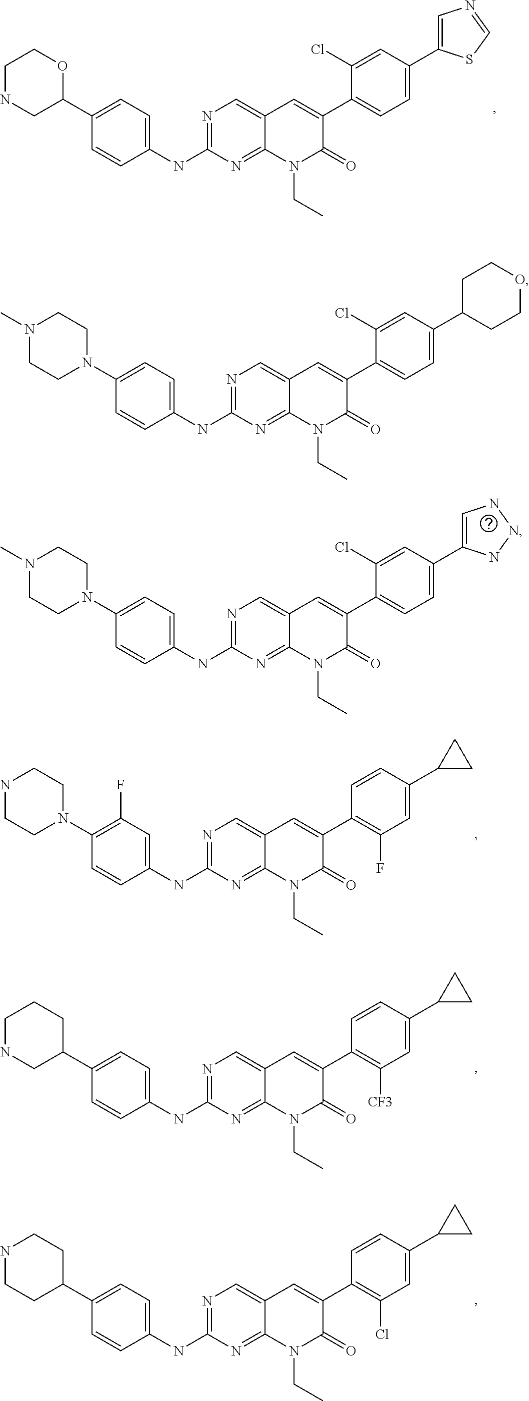

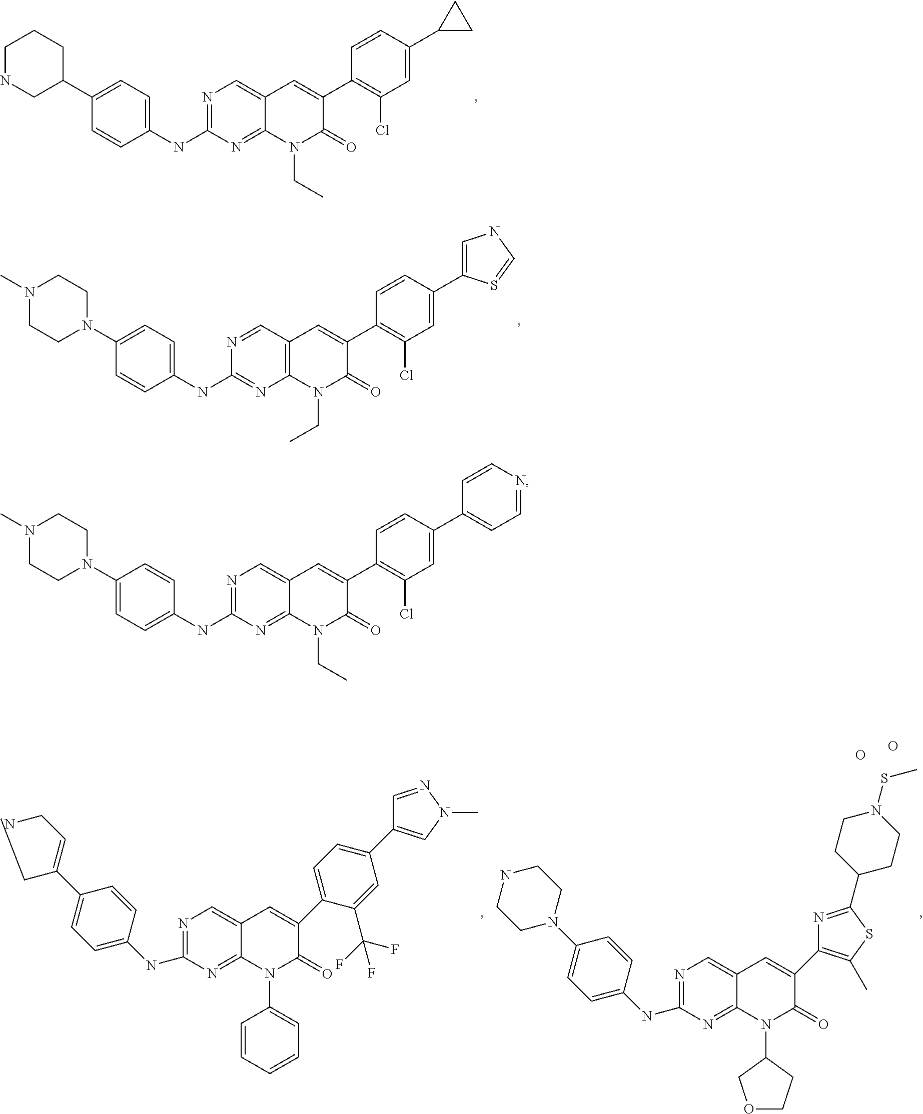

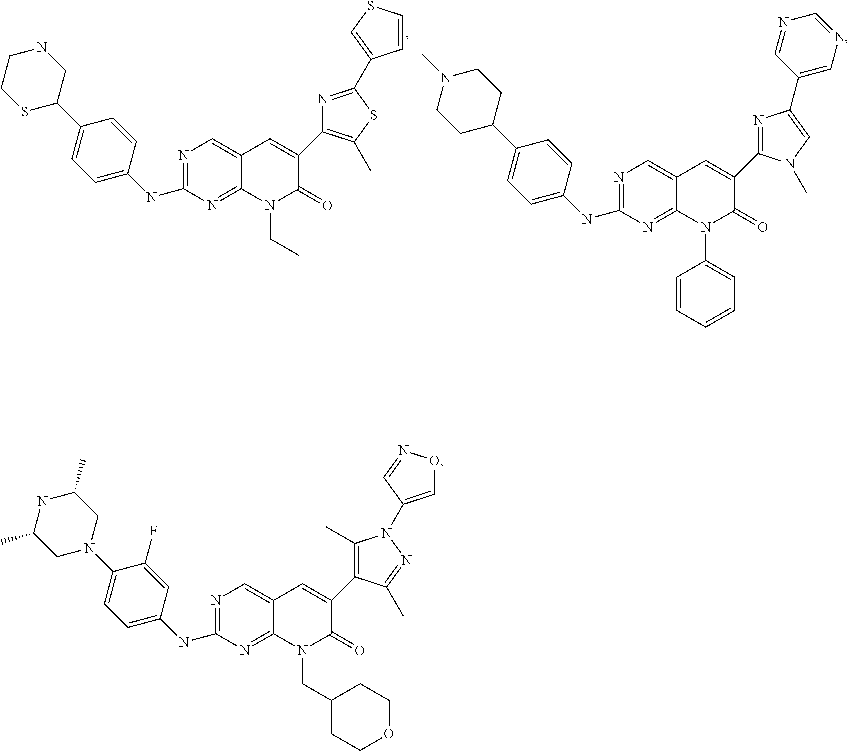

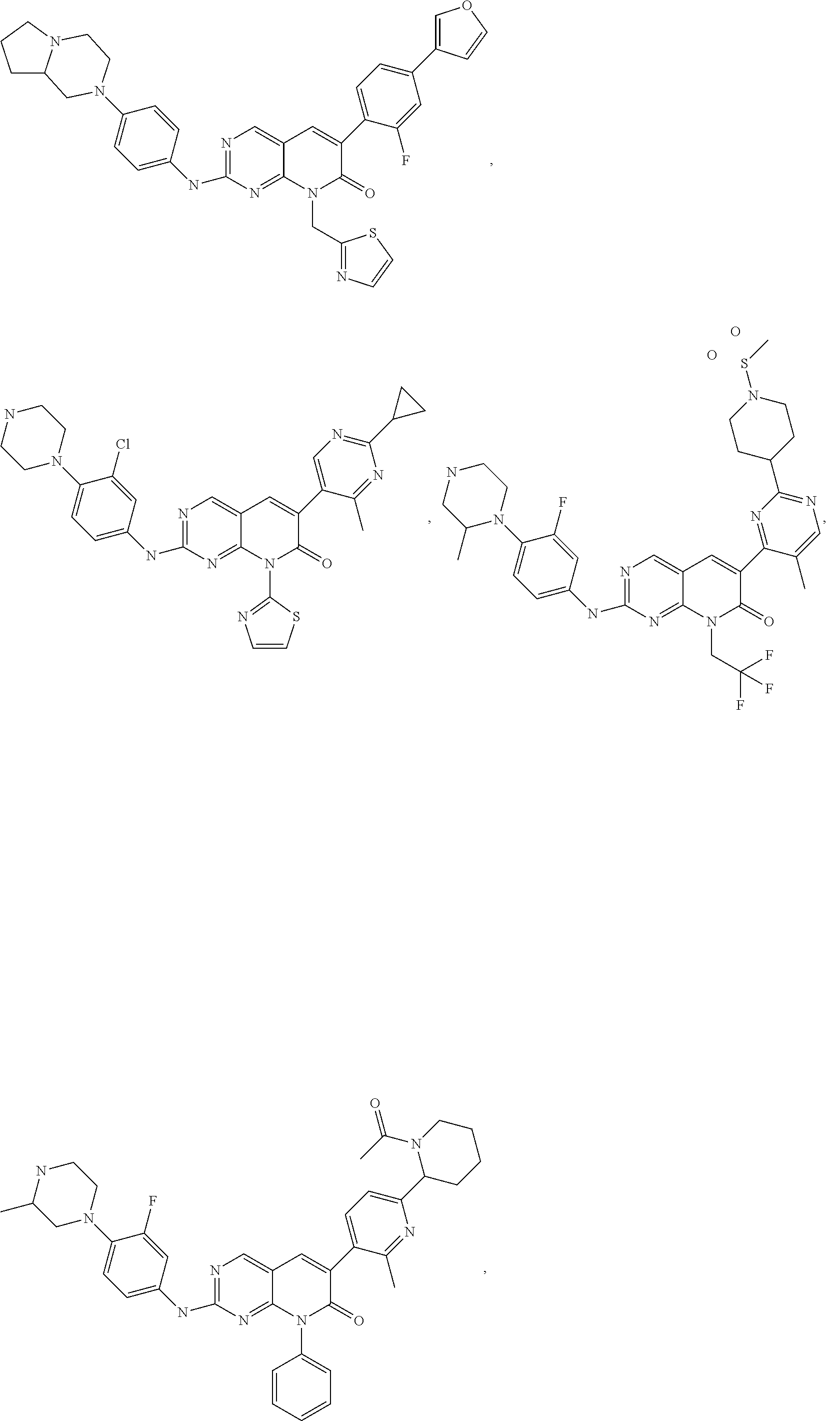

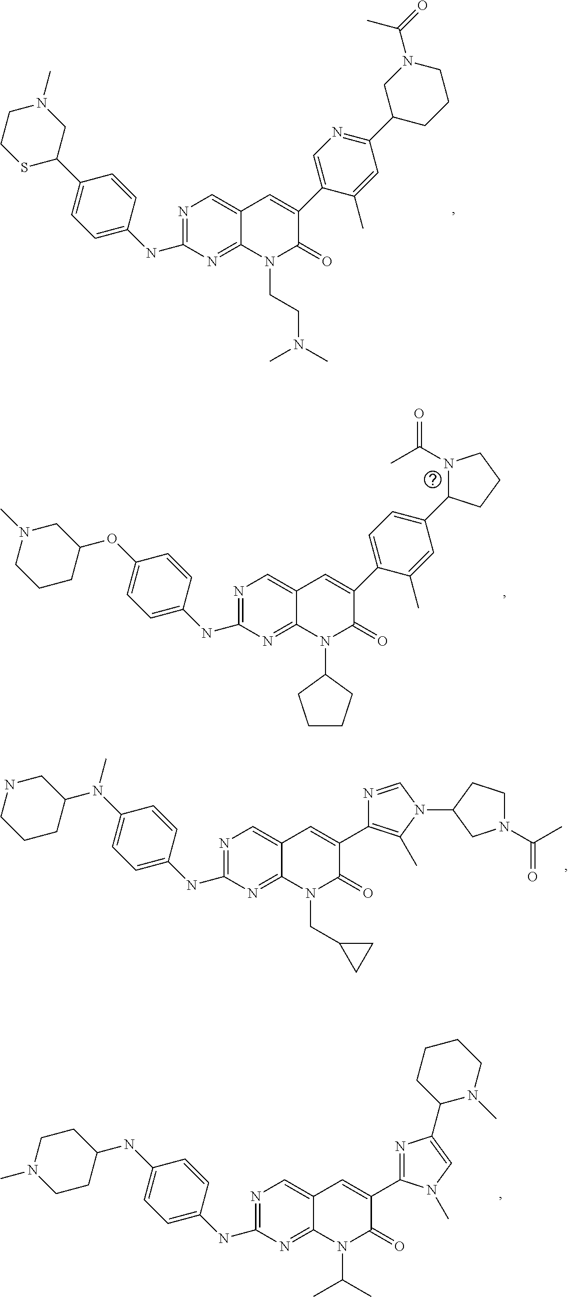

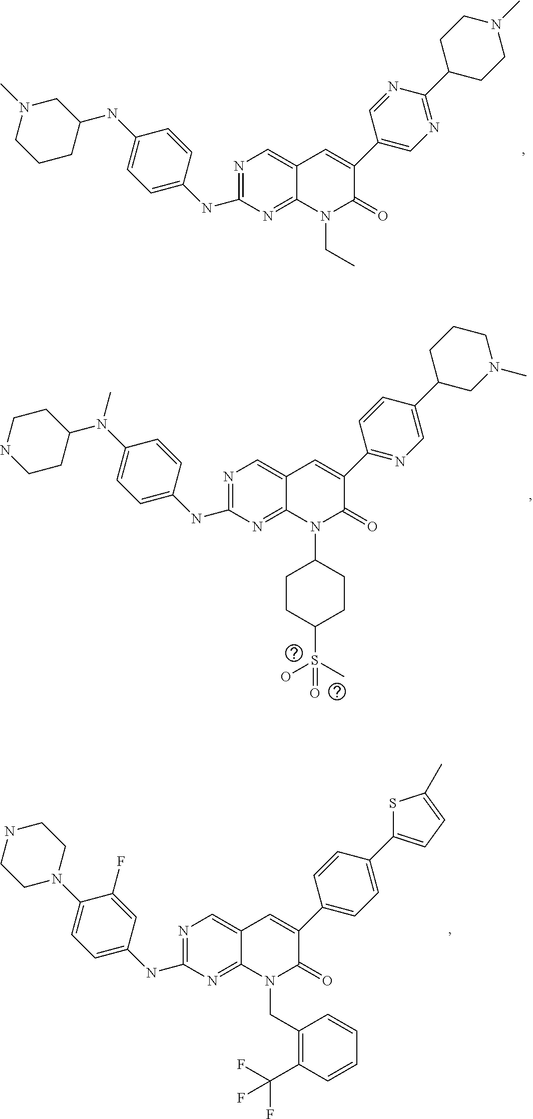

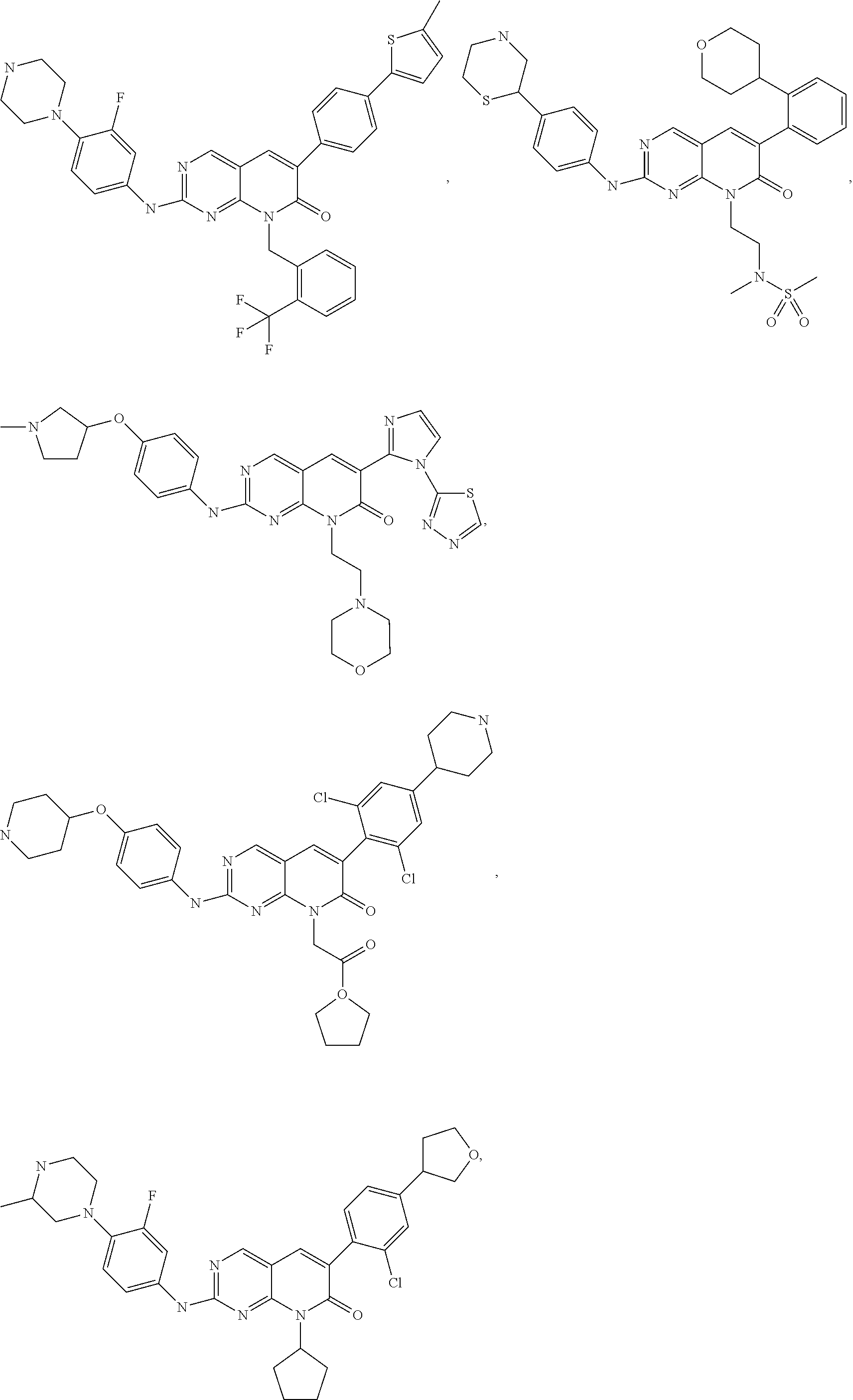

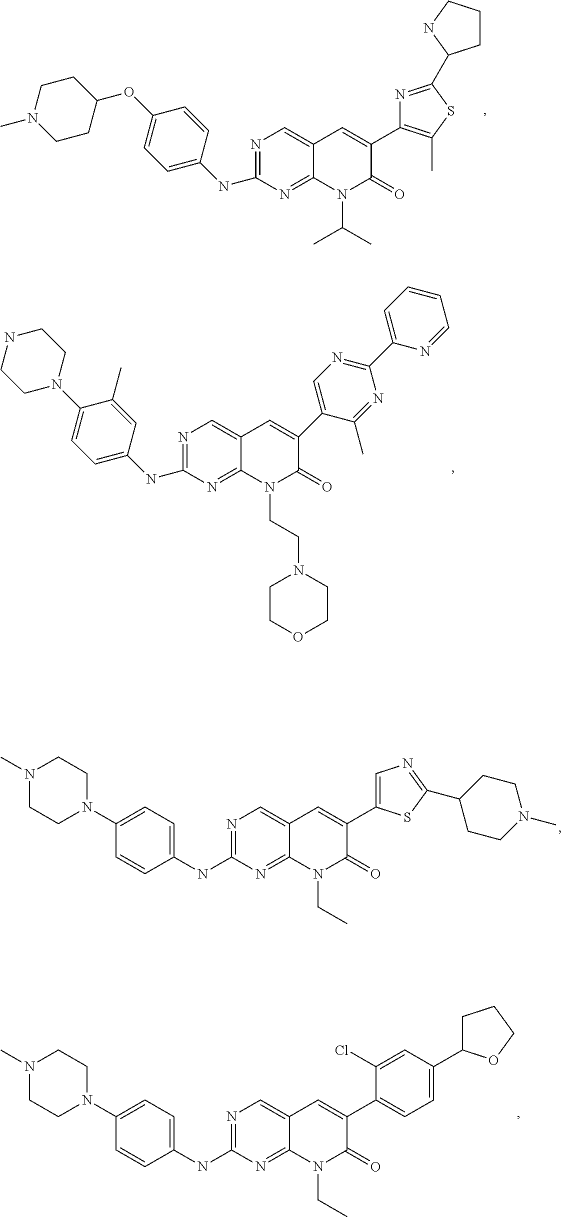

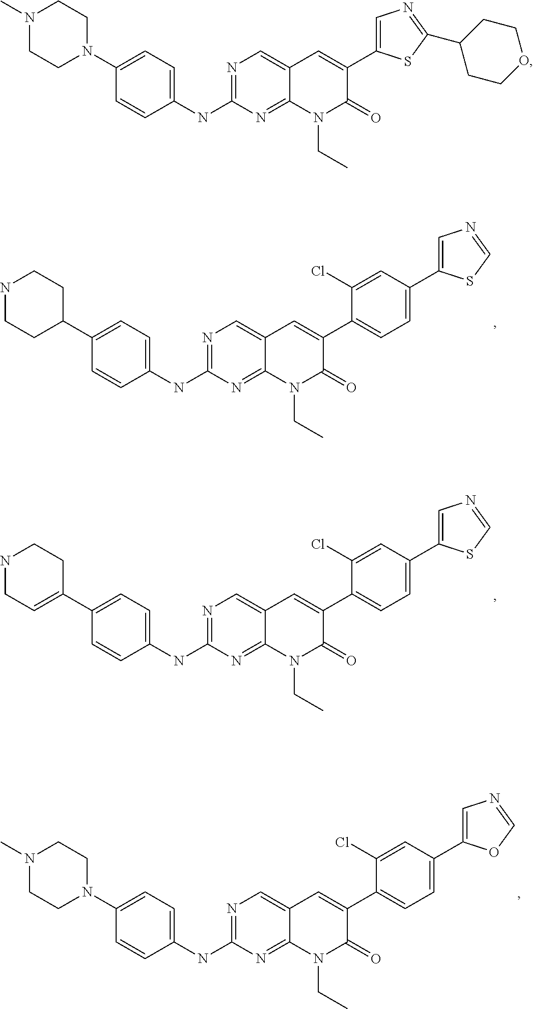 [SMILES: CCn1c(=O)c(-c2cnc(C3CCOCC3)s2)cc2cnc(Nc3ccc(N4CCN(C)CC4)cc3)nc21 ; CCn1c(=O)c(-c2ccc(-c3cncs3)cc2Cl)cc2cnc(Nc3ccc(C4CCNCC4)cc3)nc21 ; CCn1c(=O)c(-c2ccc(-c3cncs3)cc2Cl)cc2cnc(Nc3ccc(C4=CCNCC4)cc3)nc21 ; CCn1c(=O)c(-c2ccc(-c3cnco3)cc2Cl)cc2cnc(Nc3ccc(N4CCN(C)CC4)cc3)nc21]

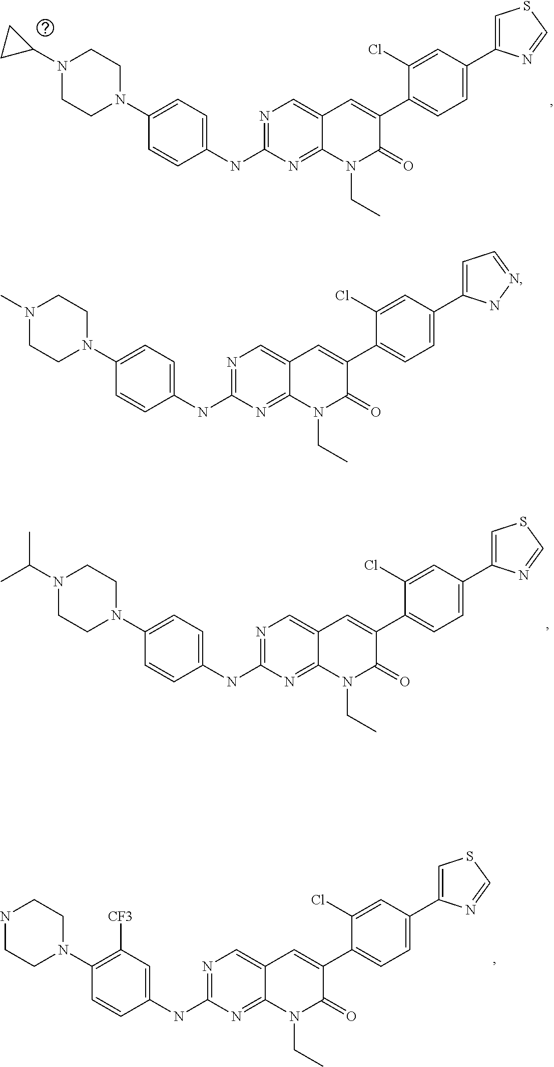 [SMILES: CCn1c(=O)c(-c2ccc(-c3cscn3)cc2Cl)cc2cnc(Nc3ccc(N4CCN(C5CC5)CC4)cc3)nc21 ; CCn1c(=O)c(-c2ccc(-c3ccn[nH]3)cc2Cl)cc2cnc(Nc3ccc(N4CCN(C)CC4)cc3)nc21 ; CCn1c(=O)c(-c2ccc(-c3cscn3)cc2Cl)cc2cnc(Nc3ccc(N4CCN(C(C)C)CC4)cc3)nc21 ; CCn1c(=O)c(-c2ccc(-c3cscn3)cc2Cl)cc2cnc(Nc3ccc(N4CCNCC4)c(C(F)(F)F)c3)nc21]

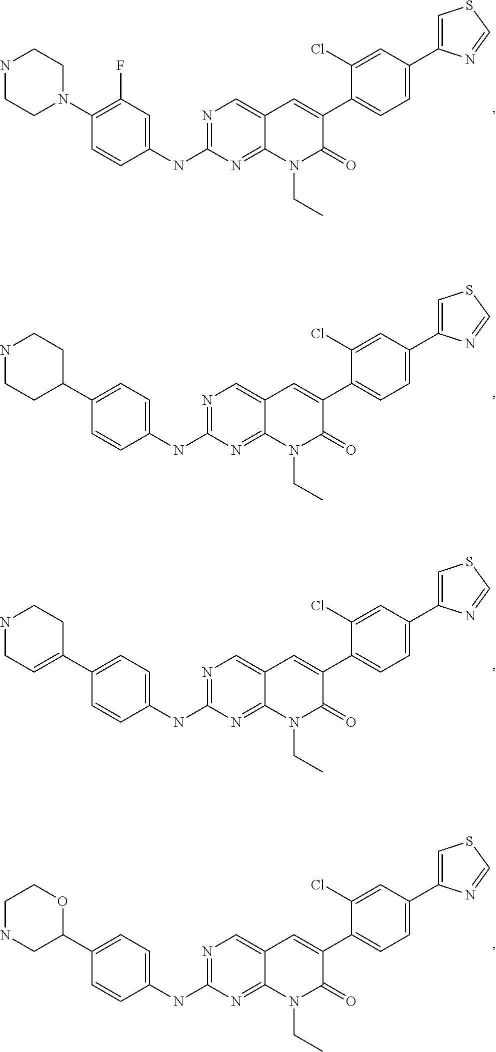 [SMILES: CCn1c(=O)c(-c2ccc(-c3cscn3)cc2Cl)cc2cnc(Nc3ccc(N4CCNCC4)c(F)c3)nc21 ; CCn1c(=O)c(-c2ccc(-c3cscn3)cc2Cl)cc2cnc(Nc3ccc(C4CCNCC4)cc3)nc21 ; CCn1c(=O)c(-c2ccc(-c3cscn3)cc2Cl)cc2cnc(Nc3ccc(C4=CCNCC4)cc3)nc21 ; CCn1c(=O)c(-c2ccc(-c3cscn3)cc2Cl)cc2cnc(Nc3ccc(C4CNCCO4)cc3)nc21]

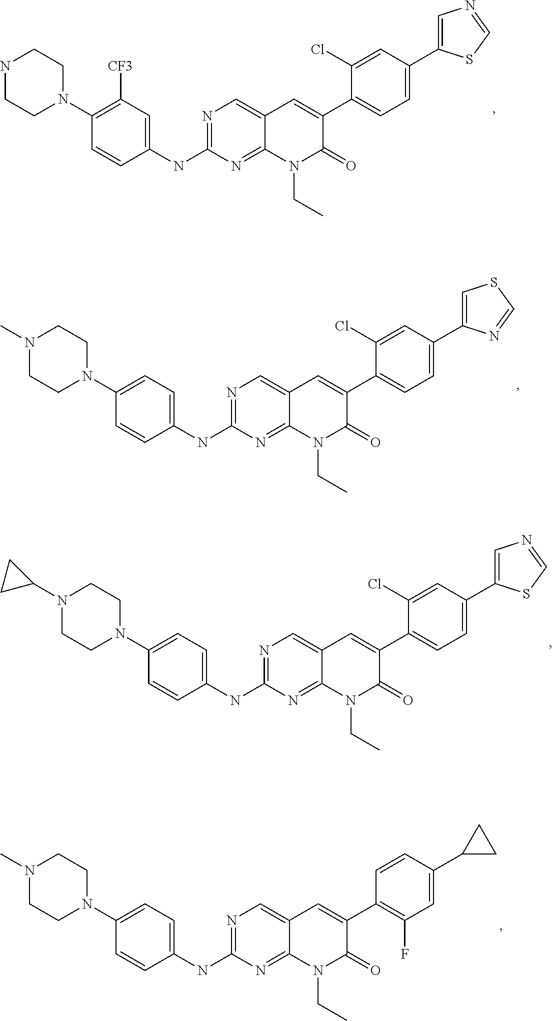 [SMILES: CCn1c(=O)c(-c2ccc(-c3cncs3)cc2Cl)cc2cnc(Nc3ccc(N4CCNCC4)c(C(F)(F)F)c3)nc21 ; CCn1c(=O)c(-c2ccc(-c3cscn3)cc2Cl)cc2cnc(Nc3ccc(N4CCN(C)CC4)cc3)nc21 ; CCn1c(=O)c(-c2ccc(-c3cncs3)cc2Cl)cc2cnc(Nc3ccc(N4CCN(C5CC5)CC4)cc3)nc21 ; CCn1c(=O)c(-c2ccc(C3CC3)cc2F)cc2cnc(Nc3ccc(N4CCN(C)CC4)cc3)nc21]

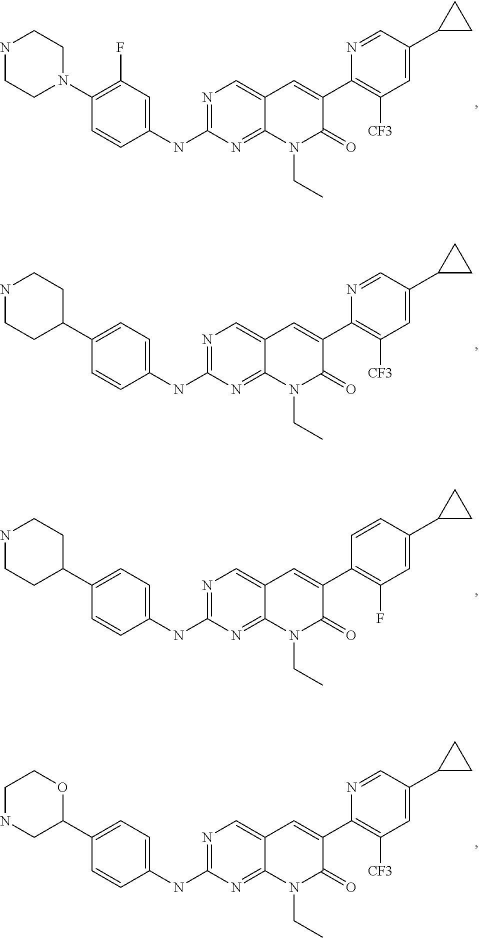 [SMILES: CCn1c(=O)c(-c2ncc(C3CC3)cc2C(F)(F)F)cc2cnc(Nc3ccc(N4CCNCC4)c(F)c3)nc21 ; CCn1c(=O)c(-c2ncc(C3CC3)cc2C(F)(F)F)cc2cnc(Nc3ccc(C4CCNCC4)cc3)nc21 ; CCn1c(=O)c(-c2ccc(C3CC3)cc2F)cc2cnc(Nc3ccc(C4CCNCC4)cc3)nc21 ; CCn1c(=O)c(-c2ncc(C3CC3)cc2C(F)(F)F)cc2cnc(Nc3ccc(C4CNCCO4)cc3)nc21]

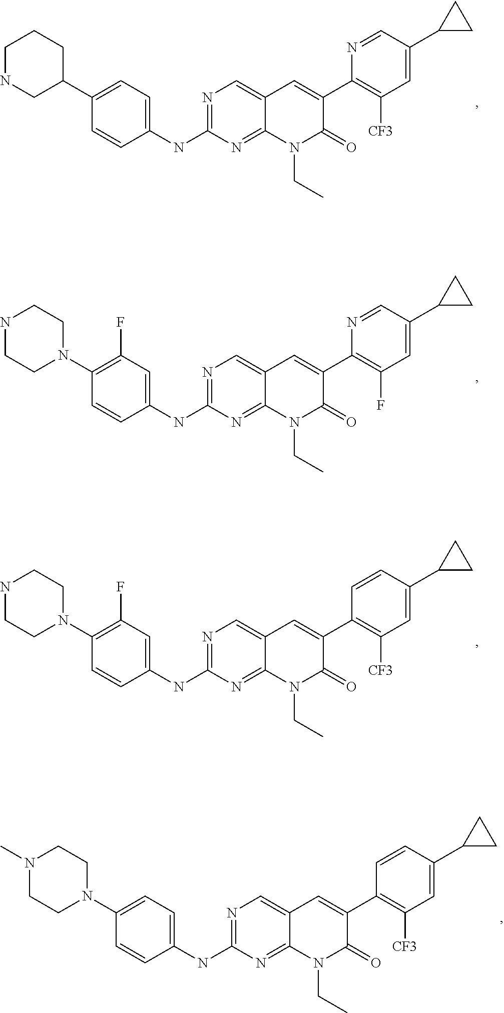 [SMILES: CCn1c(=O)c(-c2ncc(C3CC3)cc2C(F)(F)F)cc2cnc(Nc3ccc(C4CCCNC4)cc3)nc21 ; CCn1c(=O)c(-c2ncc(C3CC3)cc2F)cc2cnc(Nc3ccc(N4CCNCC4)c(F)c3)nc21 ; CCn1c(=O)c(-c2ccc(C3CC3)cc2C(F)(F)F)cc2cnc(Nc3ccc(N4CCNCC4)c(F)c3)nc21 ; CCn1c(=O)c(-c2ccc(C3CC3)cc2C(F)(F)F)cc2cnc(Nc3ccc(N4CCN(C)CC4)cc3)nc21]

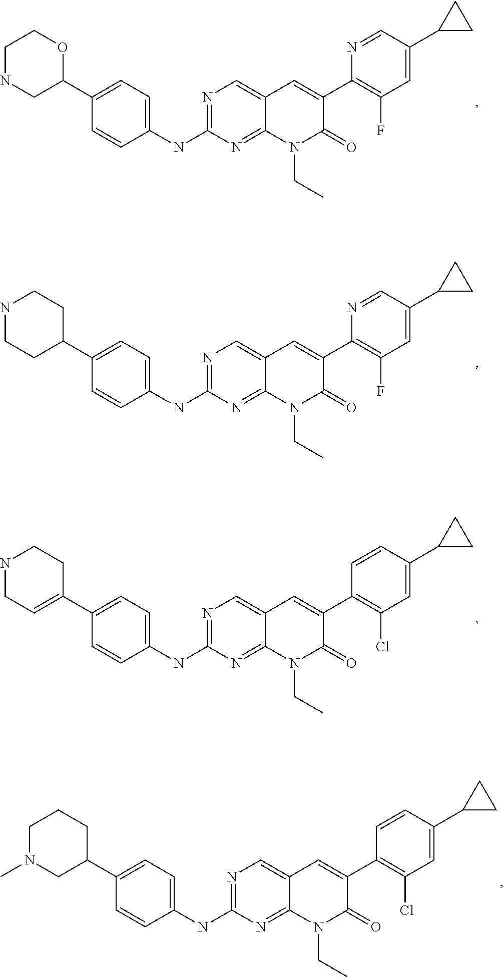 [SMILES: CCn1c(=O)c(-c2ncc(C3CC3)cc2F)cc2cnc(Nc3ccc(C4CNCCO4)cc3)nc21 ; CCn1c(=O)c(-c2ncc(C3CC3)cc2F)cc2cnc(Nc3ccc(C4CCNCC4)cc3)nc21 ; CCn1c(=O)c(-c2ccc(C3CC3)cc2Cl)cc2cnc(Nc3ccc(C4=CCNCC4)cc3)nc21 ; CCn1c(=O)c(-c2ccc(C3CC3)cc2Cl)cc2cnc(Nc3ccc(C4CCCN(C)C4)cc3)nc21]

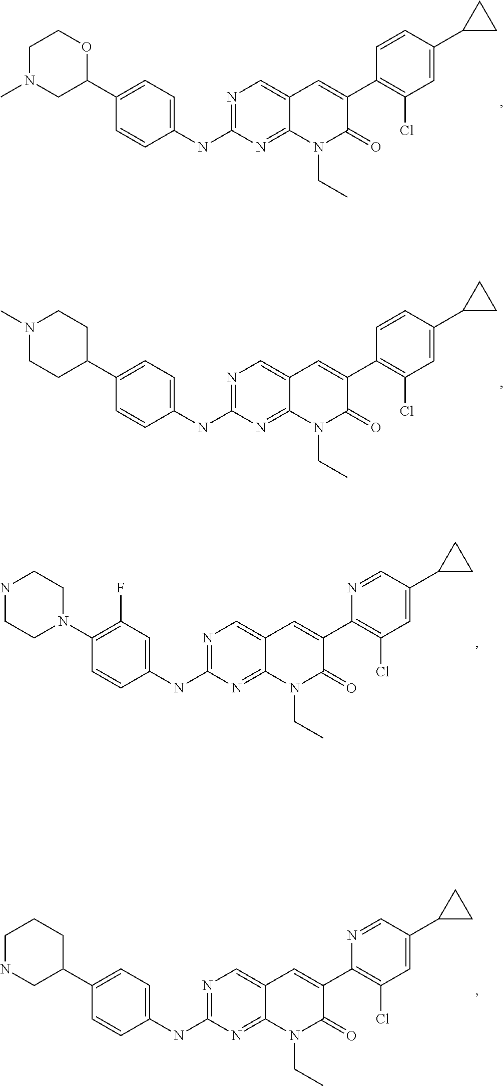 [SMILES: CCn1c(=O)c(-c2ccc(C3CC3)cc2Cl)cc2cnc(Nc3ccc(C4CN(C)CCO4)cc3)nc21 ; CCn1c(=O)c(-c2ccc(C3CC3)cc2Cl)cc2cnc(Nc3ccc(C4CCN(C)CC4)cc3)nc21 ; CCn1c(=O)c(-c2ncc(C3CC3)cc2Cl)cc2cnc(Nc3ccc(N4CCNCC4)c(F)c3)nc21 ; CCn1c(=O)c(-c2ncc(C3CC3)cc2Cl)cc2cnc(Nc3ccc(C4CCCNC4)cc3)nc21]

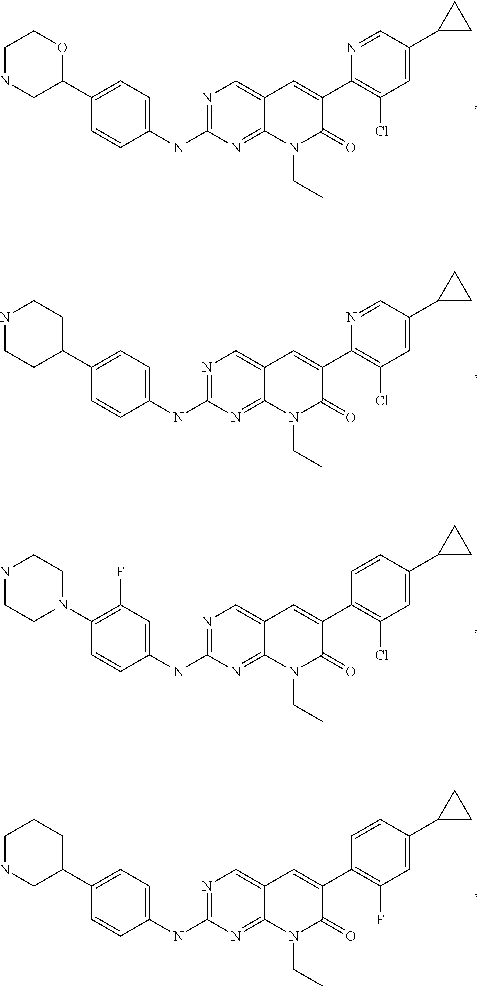 [SMILES: CCn1c(=O)c(-c2ncc(C3CC3)cc2Cl)cc2cnc(Nc3ccc(C4CNCCO4)cc3)nc21 ; CCn1c(=O)c(-c2ncc(C3CC3)cc2Cl)cc2cnc(Nc3ccc(C4CCNCC4)cc3)nc21 ; CCn1c(=O)c(-c2ccc(C3CC3)cc2Cl)cc2cnc(Nc3ccc(N4CCNCC4)c(F)c3)nc21 ; CCn1c(=O)c(-c2ccc(C3CC3)cc2F)cc2cnc(Nc3ccc(C4CCCNC4)cc3)nc21]

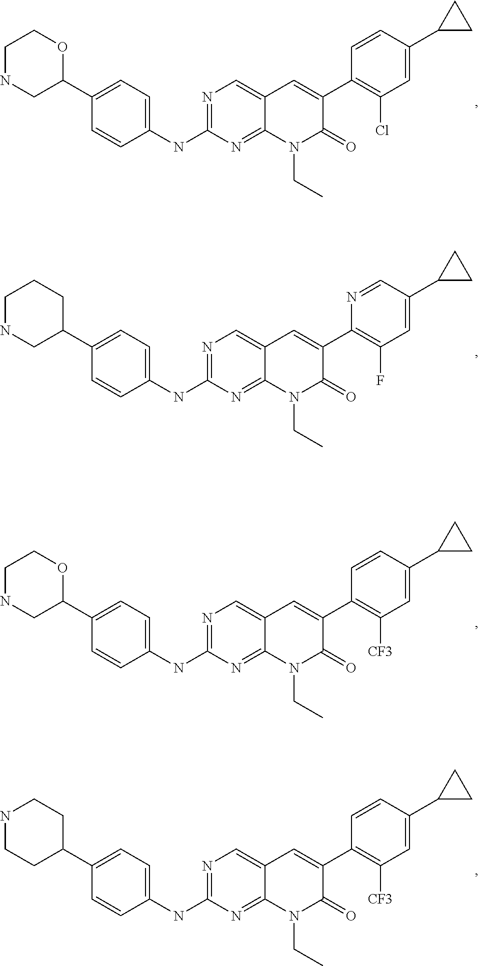 [SMILES: CCn1c(=O)c(-c2ccc(C3CC3)cc2Cl)cc2cnc(Nc3ccc(C4CNCCO4)cc3)nc21 ; CCn1c(=O)c(-c2ncc(C3CC3)cc2F)cc2cnc(Nc3ccc(C4CCCNC4)cc3)nc21 ; CCn1c(=O)c(-c2ccc(C3CC3)cc2C(F)(F)F)cc2cnc(Nc3ccc(C4CNCCO4)cc3)nc21 ; CCn1c(=O)c(-c2ccc(C3CC3)cc2C(F)(F)F)cc2cnc(Nc3ccc(C4CCNCC4)cc3)nc21]

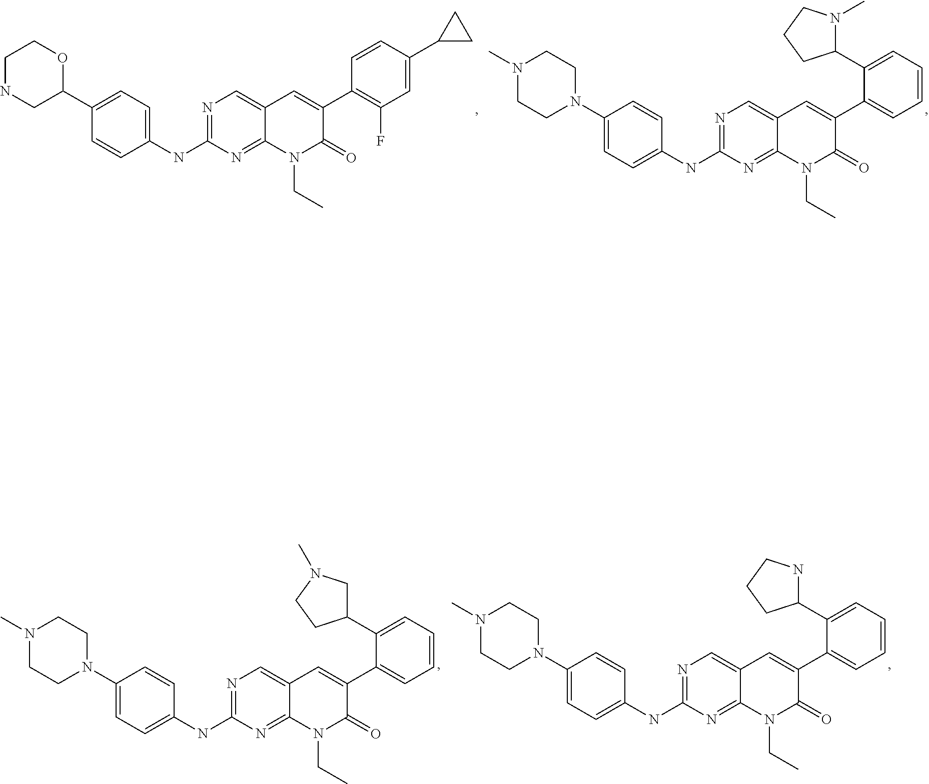

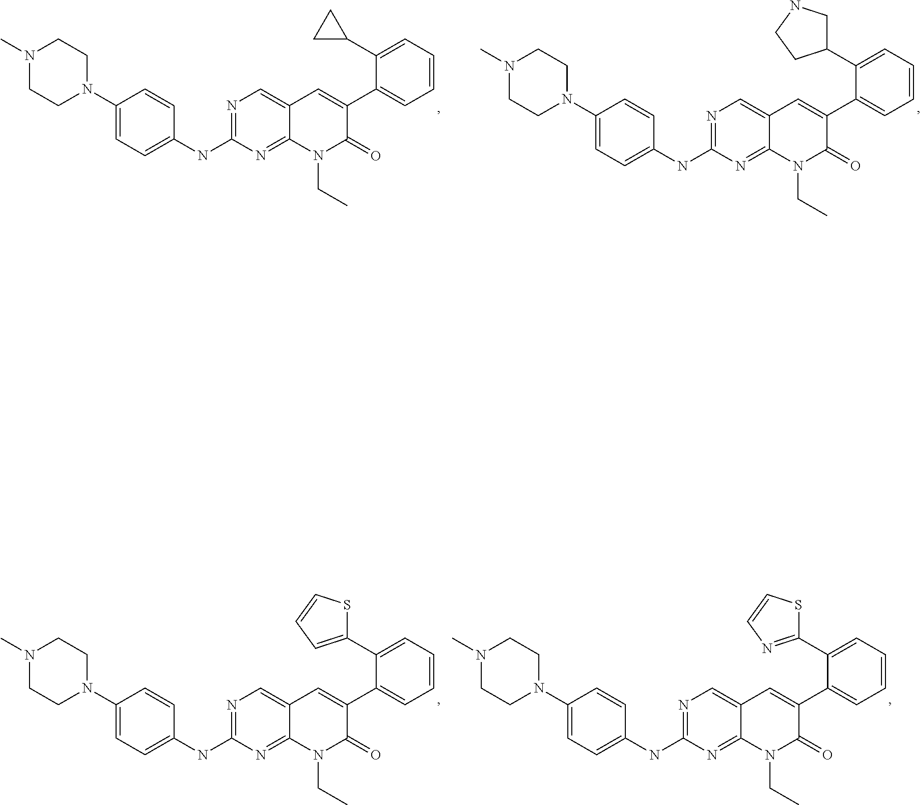

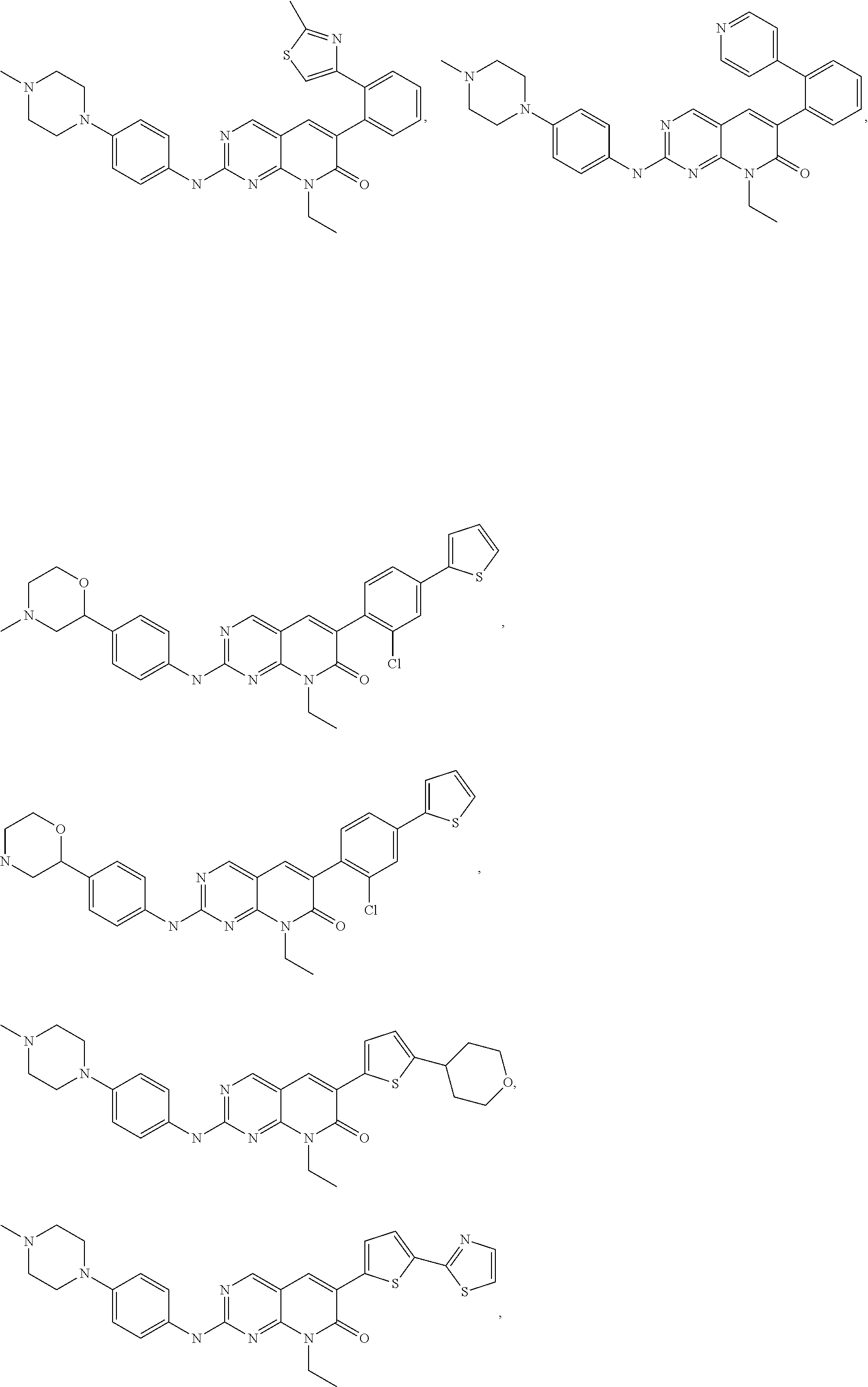

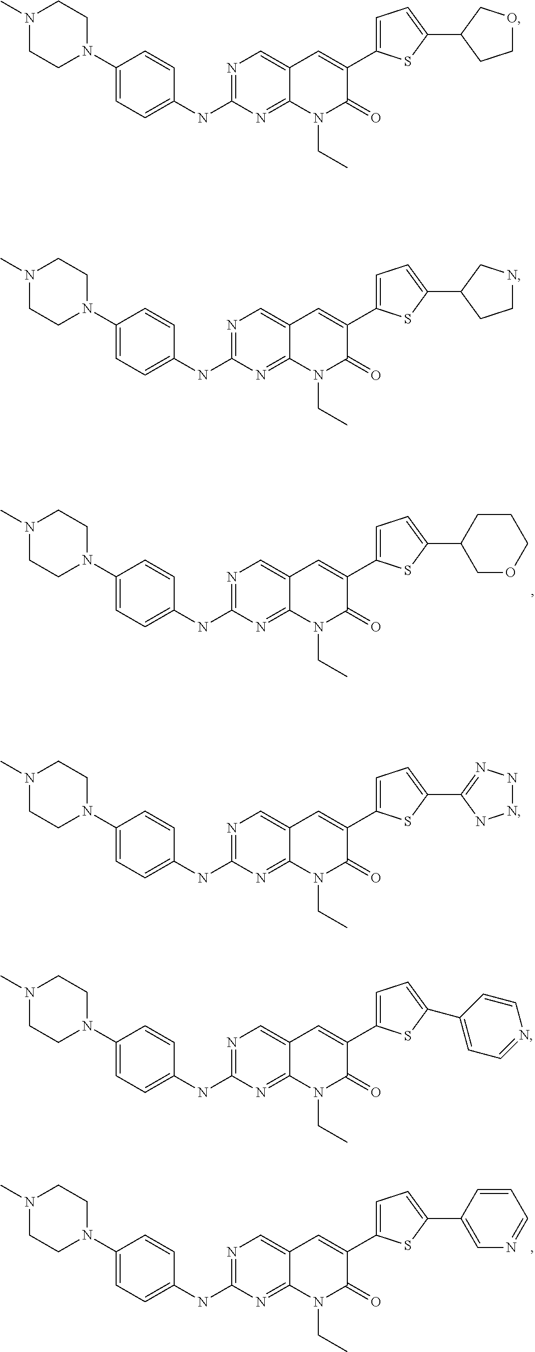

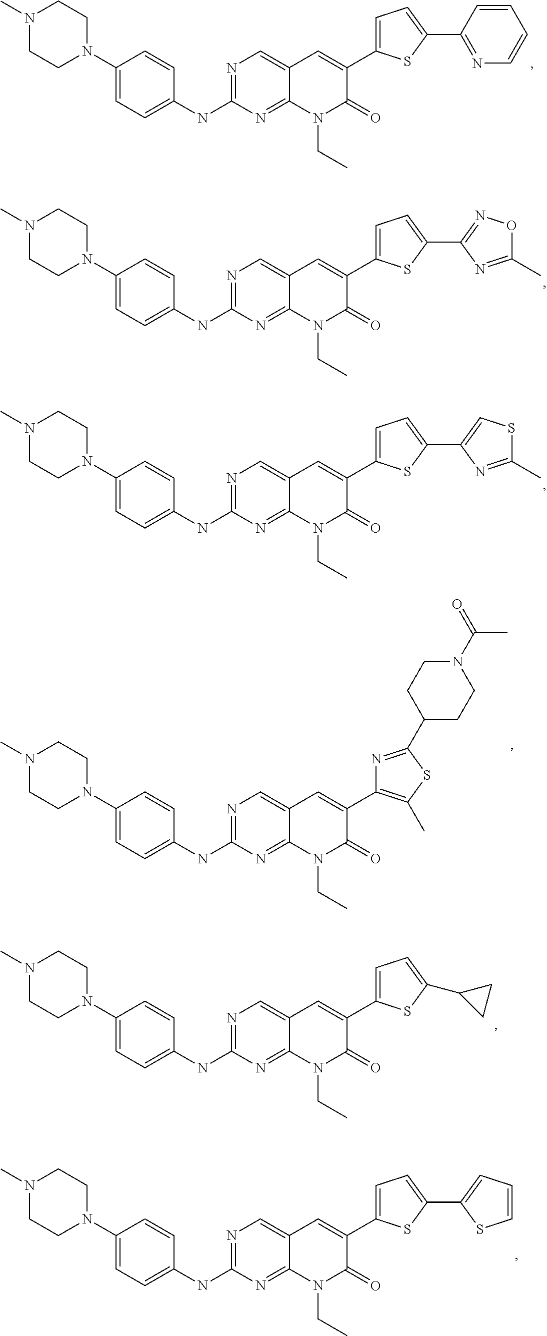

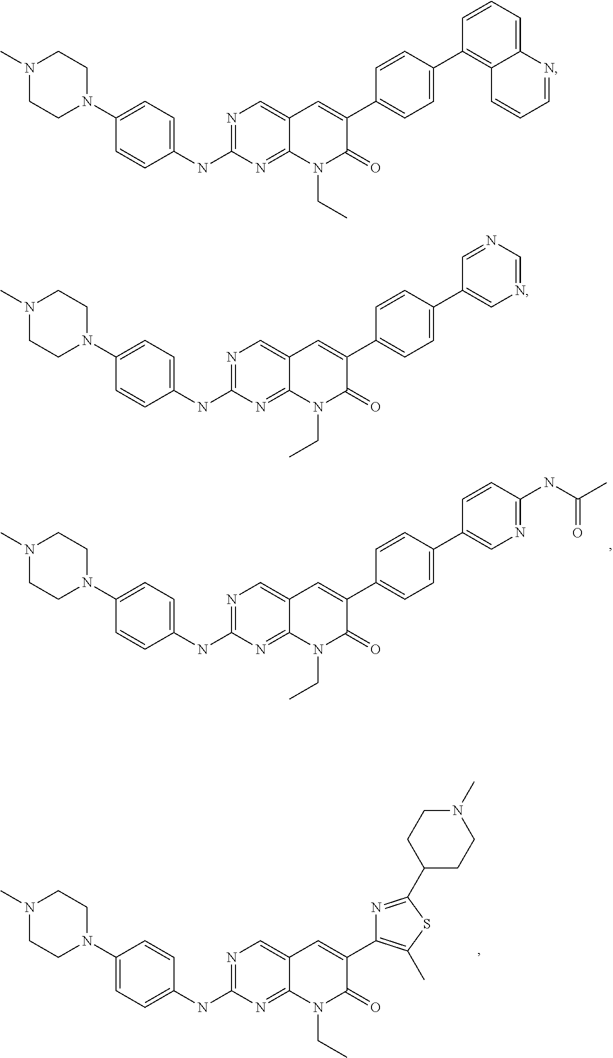

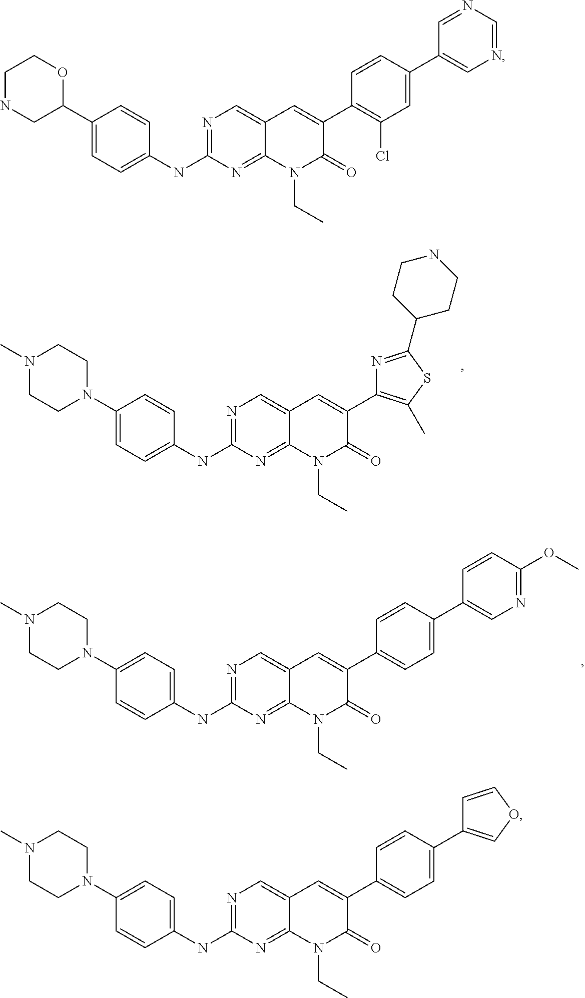

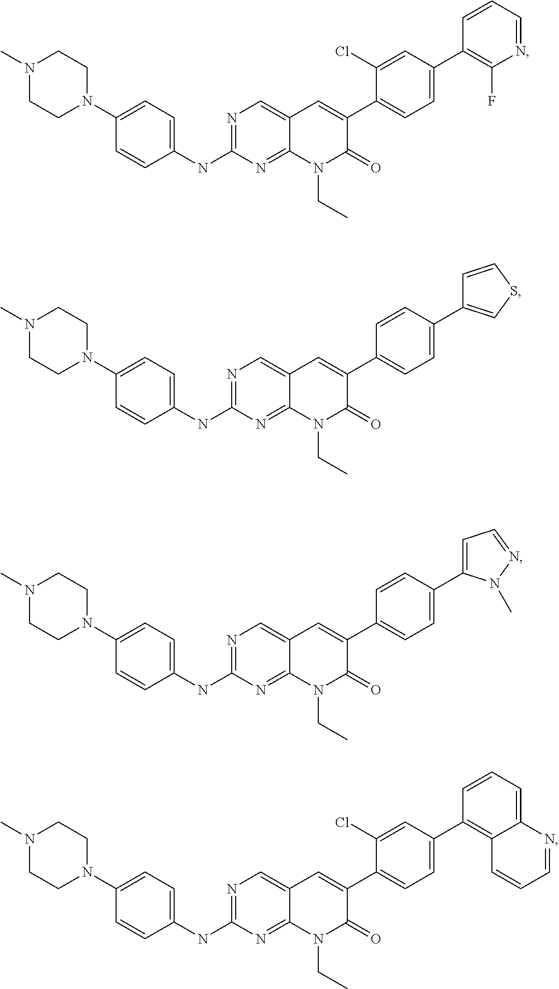 [SMILES: CCn1c(=O)c(-c2ccc(-c3cccnc3F)cc2Cl)cc2cnc(Nc3ccc(N4CCN(C)CC4)cc3)nc21 ; CCn1c(=O)c(-c2ccc(-c3ccsc3)cc2)cc2cnc(Nc3ccc(N4CCN(C)CC4)cc3)nc21 ; CCn1c(=O)c(-c2ccc(-c3ccnn3C)cc2)cc2cnc(Nc3ccc(N4CCN(C)CC4)cc3)nc21 ; CCn1c(=O)c(-c2ccc(-c3cccc4ncccc34)cc2Cl)cc2cnc(Nc3ccc(N4CCN(C)CC4)cc3)nc21]

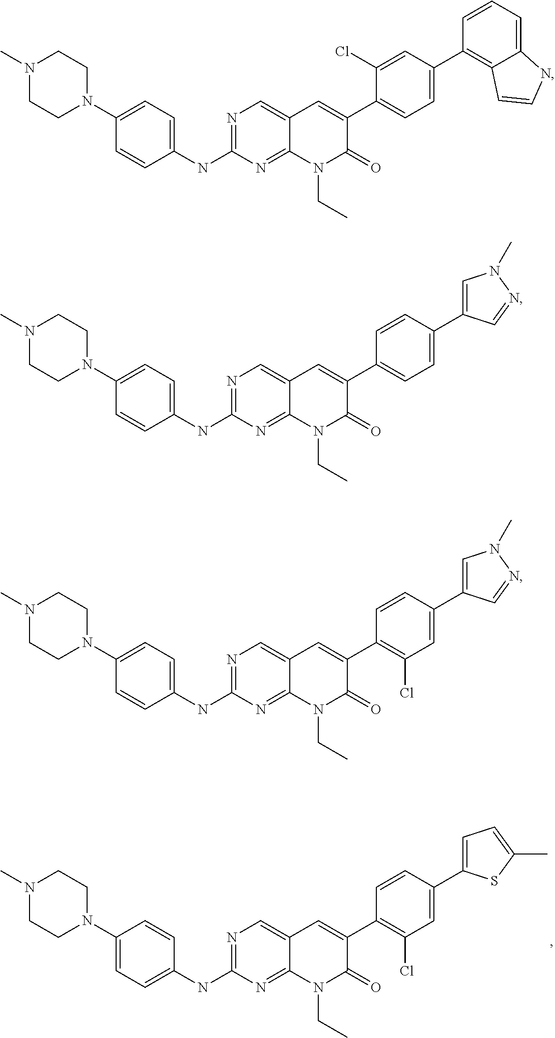

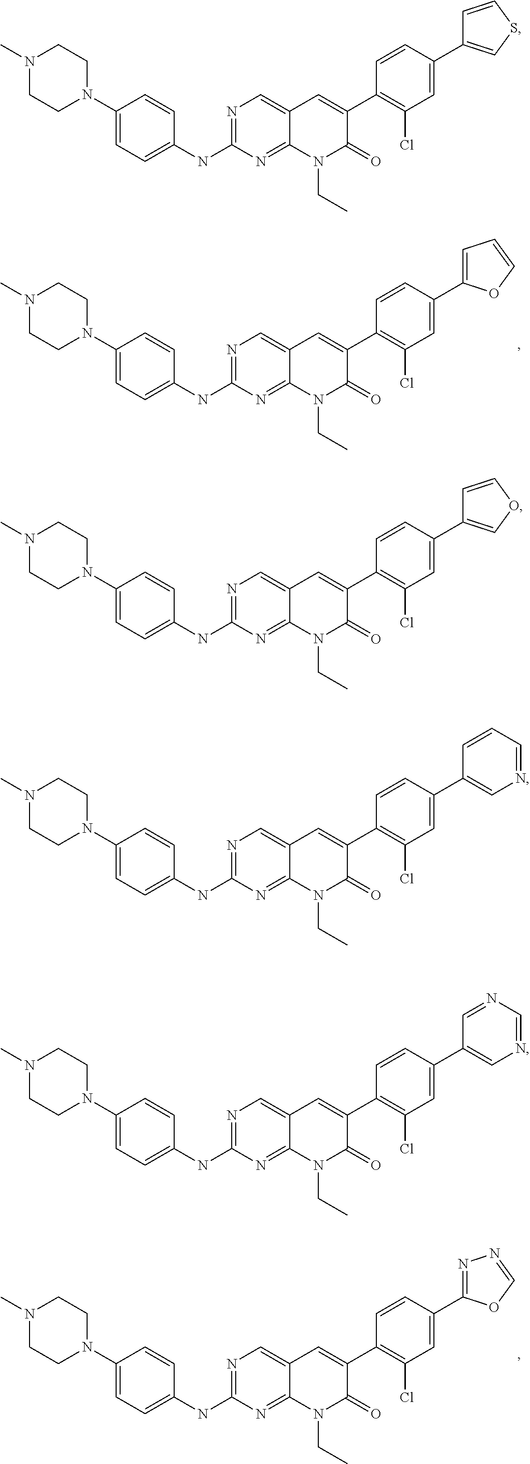 [SMILES: CCn1c(=O)c(-c2ccc(-c3ccsc3)cc2Cl)cc2cnc(Nc3ccc(N4CCN(C)CC4)cc3)nc21 ; CCn1c(=O)c(-c2ccc(-c3ccco3)cc2Cl)cc2cnc(Nc3ccc(N4CCN(C)CC4)cc3)nc21 ; CCn1c(=O)c(-c2ccc(-c3ccoc3)cc2Cl)cc2cnc(Nc3ccc(N4CCN(C)CC4)cc3)nc21 ; CCn1c(=O)c(-c2ccc(-c3cccnc3)cc2Cl)cc2cnc(Nc3ccc(N4CCN(C)CC4)cc3)nc21 ; CCn1c(=O)c(-c2ccc(-c3cncnc3)cc2Cl)cc2cnc(Nc3ccc(N4CCN(C)CC4)cc3)nc21 ; CCn1c(=O)c(-c2ccc(-c3nnco3)cc2Cl)cc2cnc(Nc3ccc(N4CCN(C)CC4)cc3)nc21]

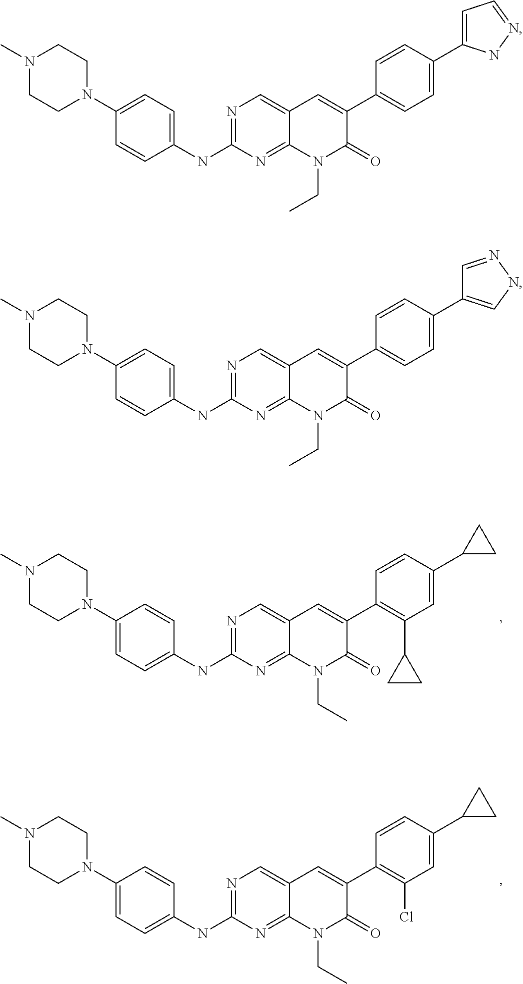

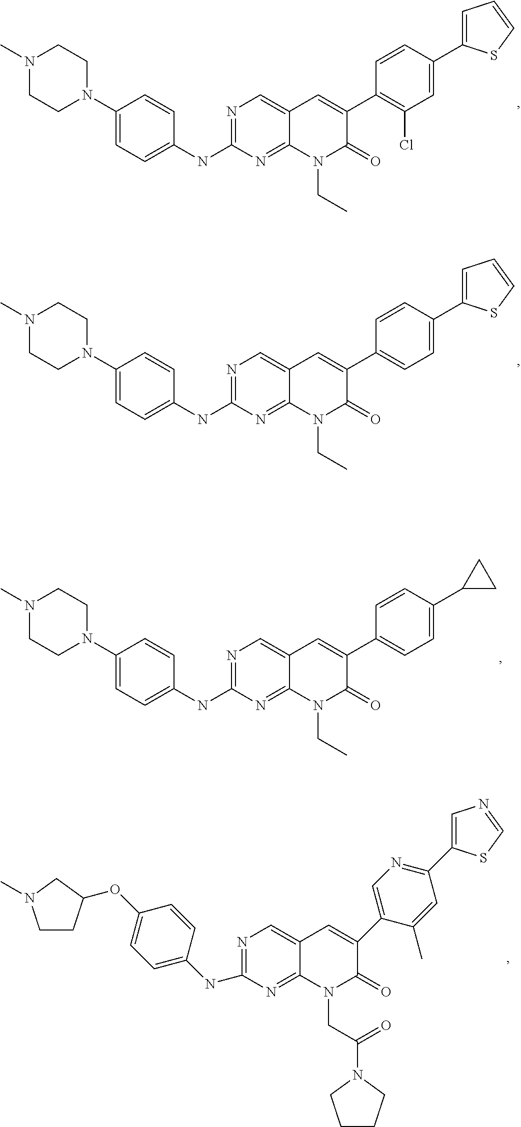 [SMILES: CCn1c(=O)c(-c2ccc(-c3cccs3)cc2Cl)cc2cnc(Nc3ccc(N4CCN(C)CC4)cc3)nc21 ; CCn1c(=O)c(-c2ccc(-c3cccs3)cc2)cc2cnc(Nc3ccc(N4CCN(C)CC4)cc3)nc21 ; CCn1c(=O)c(-c2ccc(C3CC3)cc2)cc2cnc(Nc3ccc(N4CCN(C)CC4)cc3)nc21 ; Cc1cc(-c2cncs2)ncc1-c1cc2cnc(Nc3ccc(OC4CCN(C)C4)cc3)nc2n(CC(=O)N2CCCC2)c1=O]

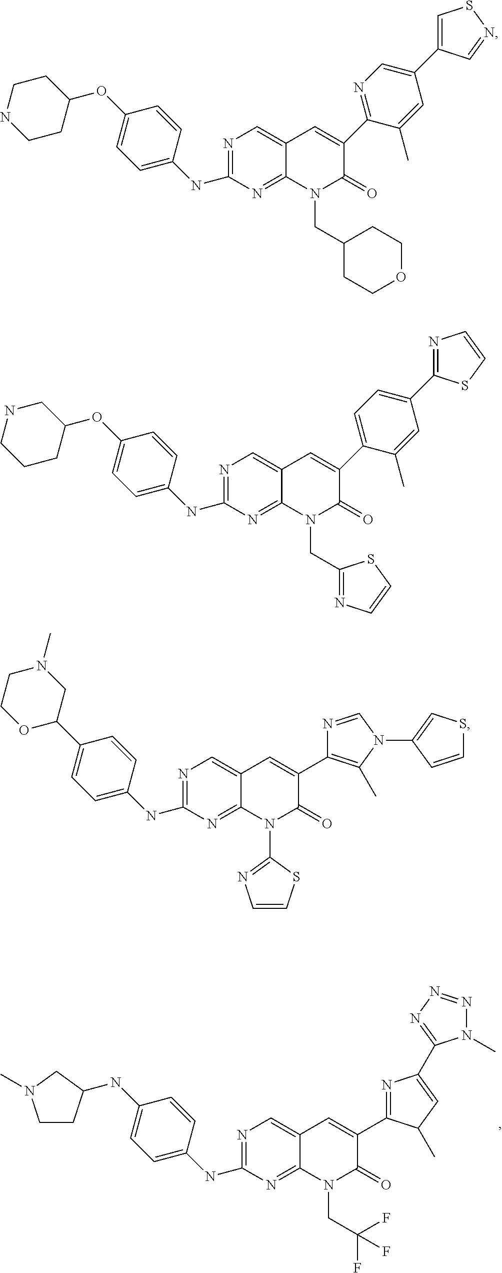

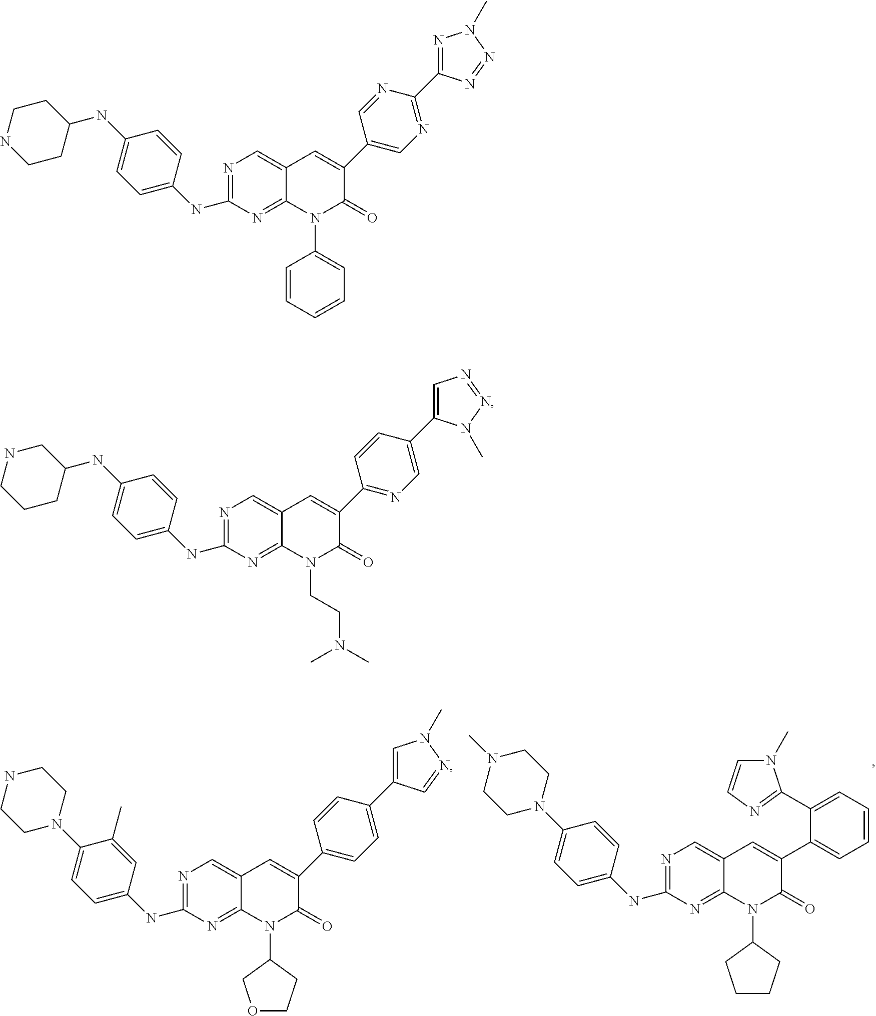

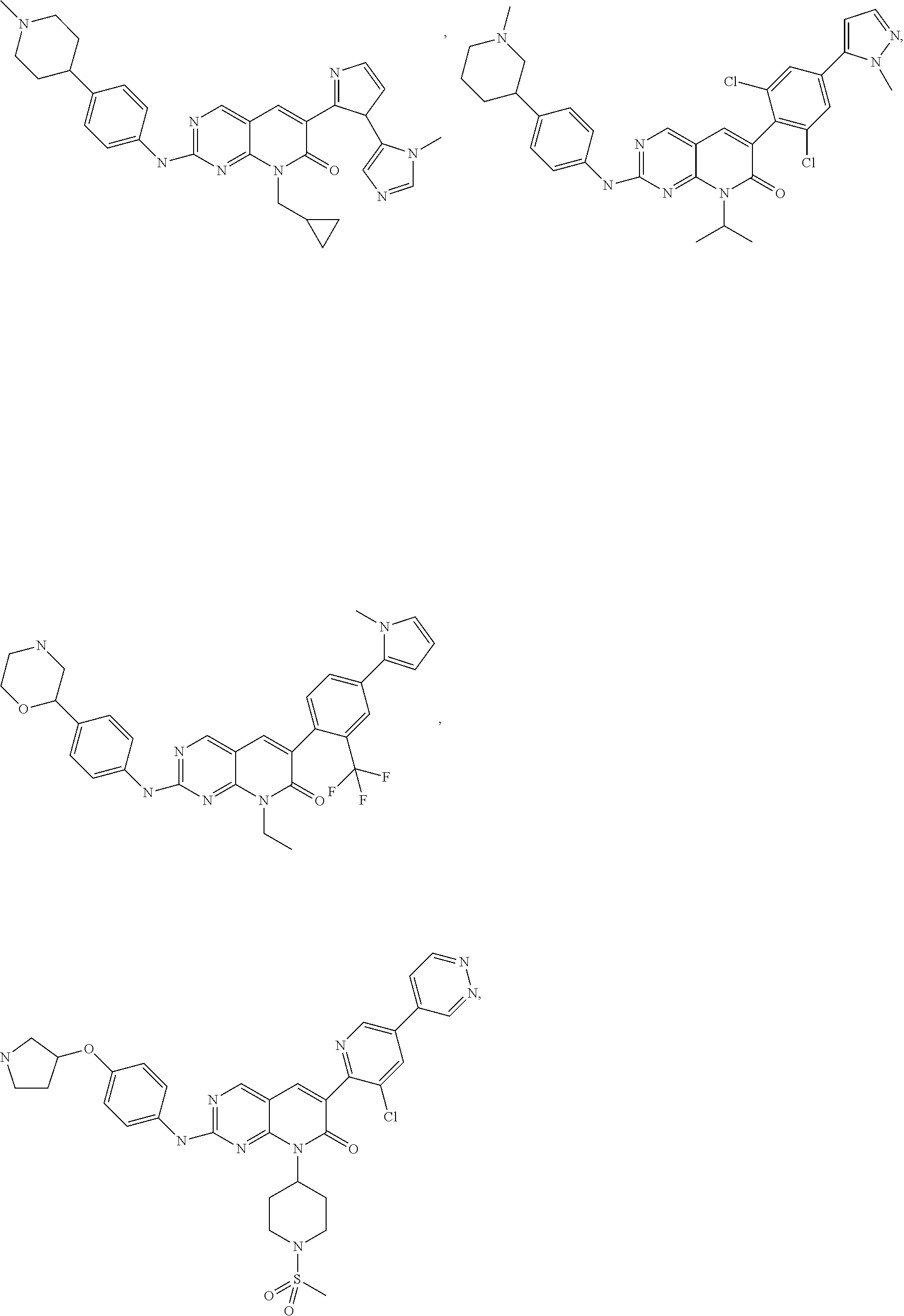

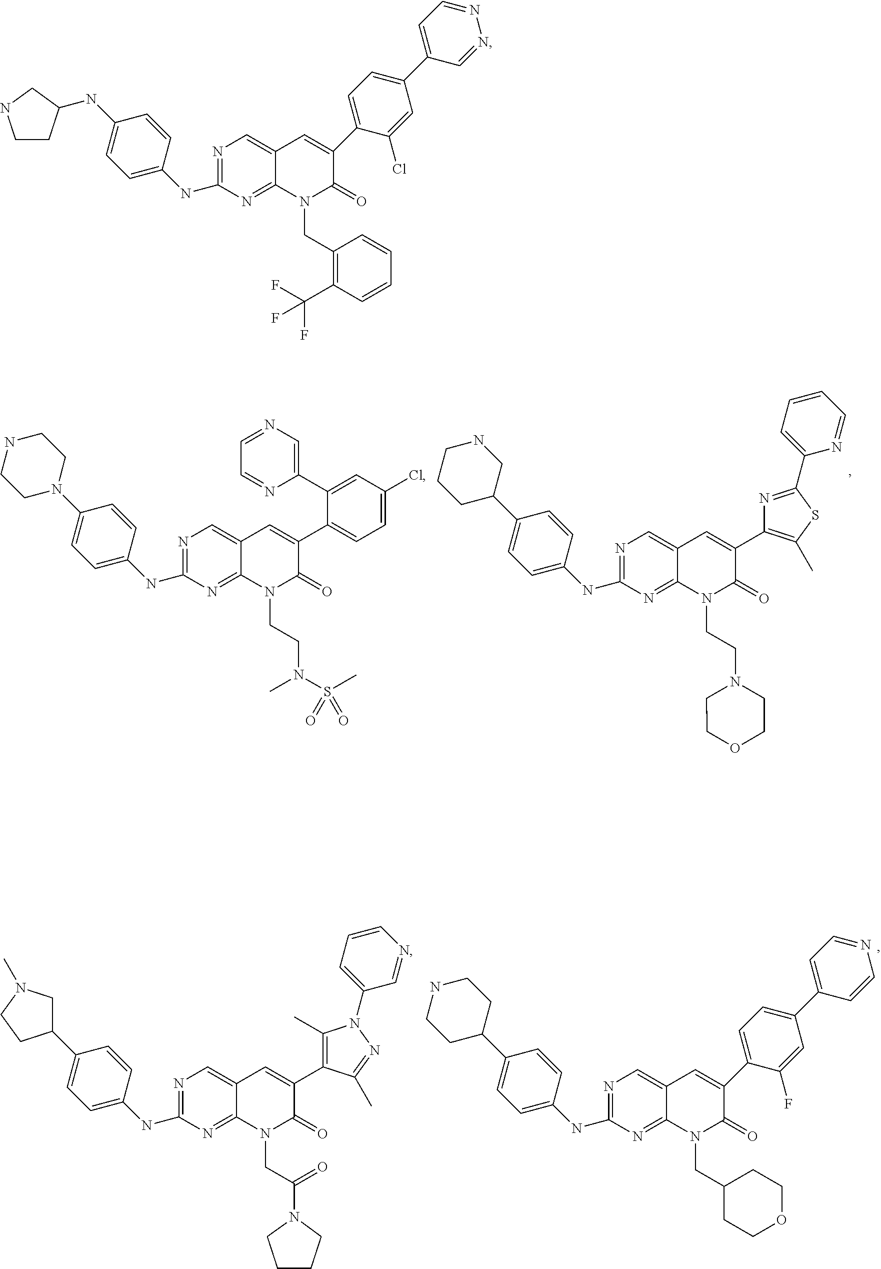

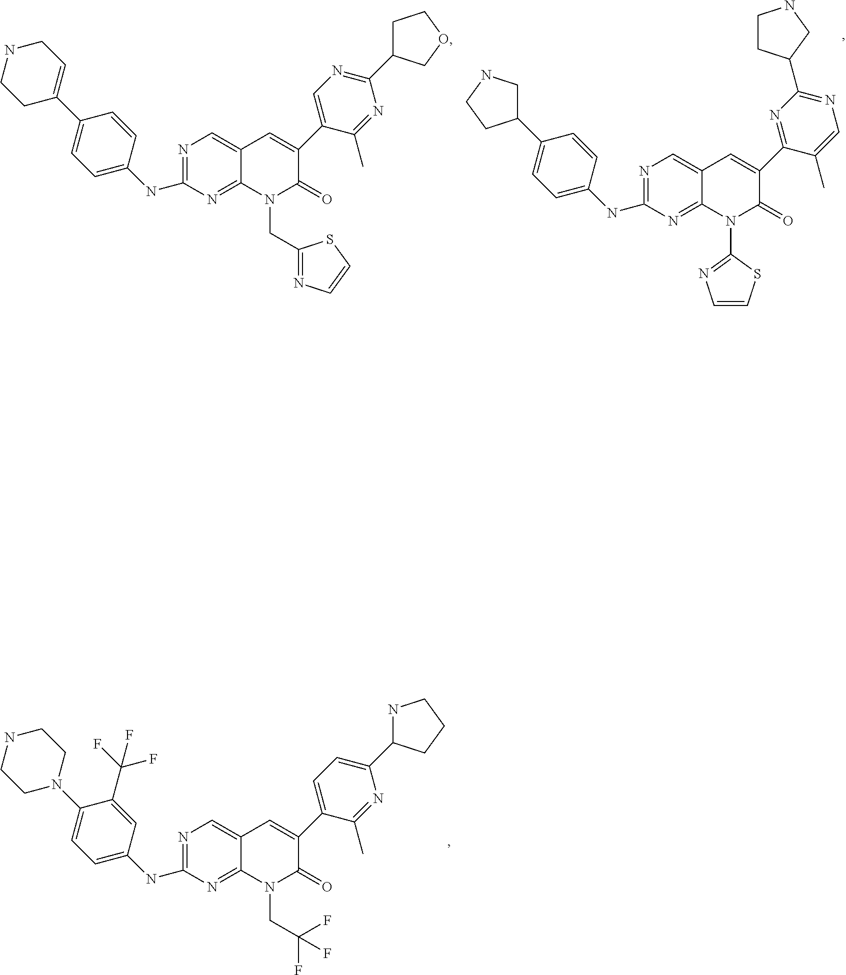 [SMILES: Cc1nc(C2CCOC2)ncc1-c1cc2cnc(Nc3ccc(C4=CCNCC4)cc3)nc2n(Cc2nccs2)c1=O ; Cc1cnc(C2CCNC2)nc1-c1cc2cnc(Nc3ccc(C4CCNC4)cc3)nc2n(-c2nccs2)c1=O ; "," ; Cc1nc(C2CCCN2)ccc1-c1cc2cnc(Nc3ccc(N4CCNCC4)c(C(F)(F)F)c3)nc2n(CC(F)(F)F)c1=O]

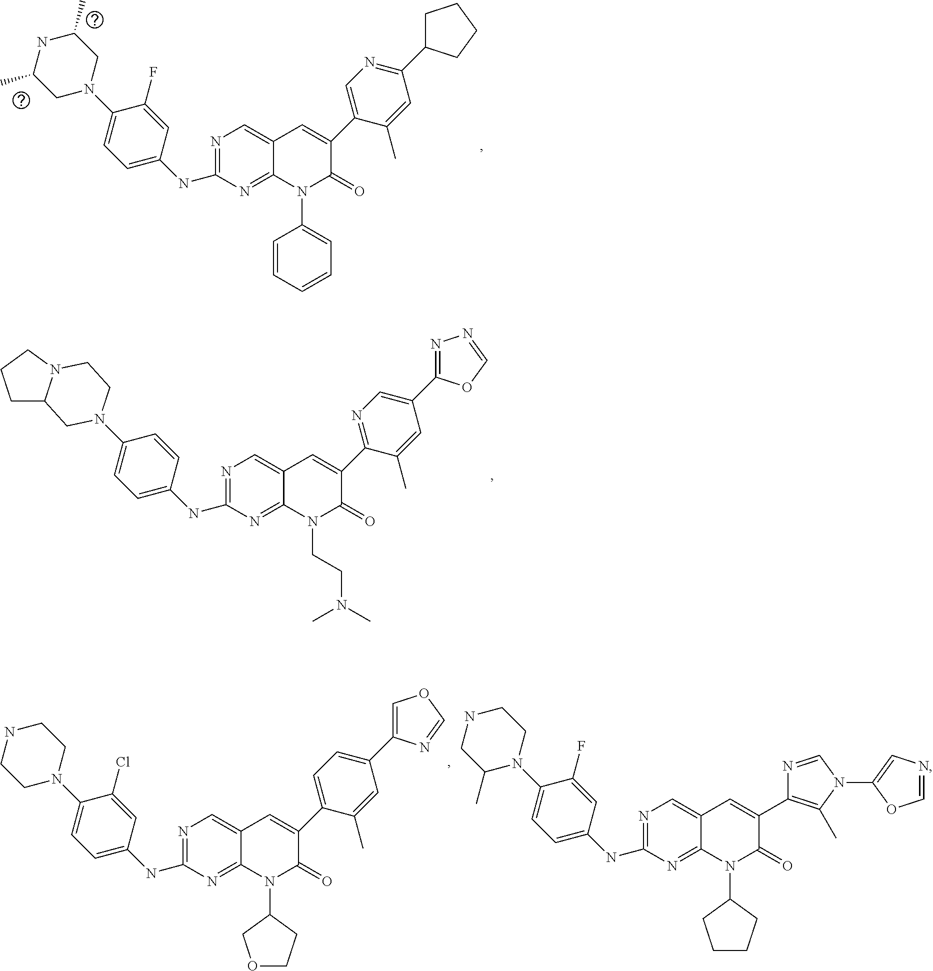

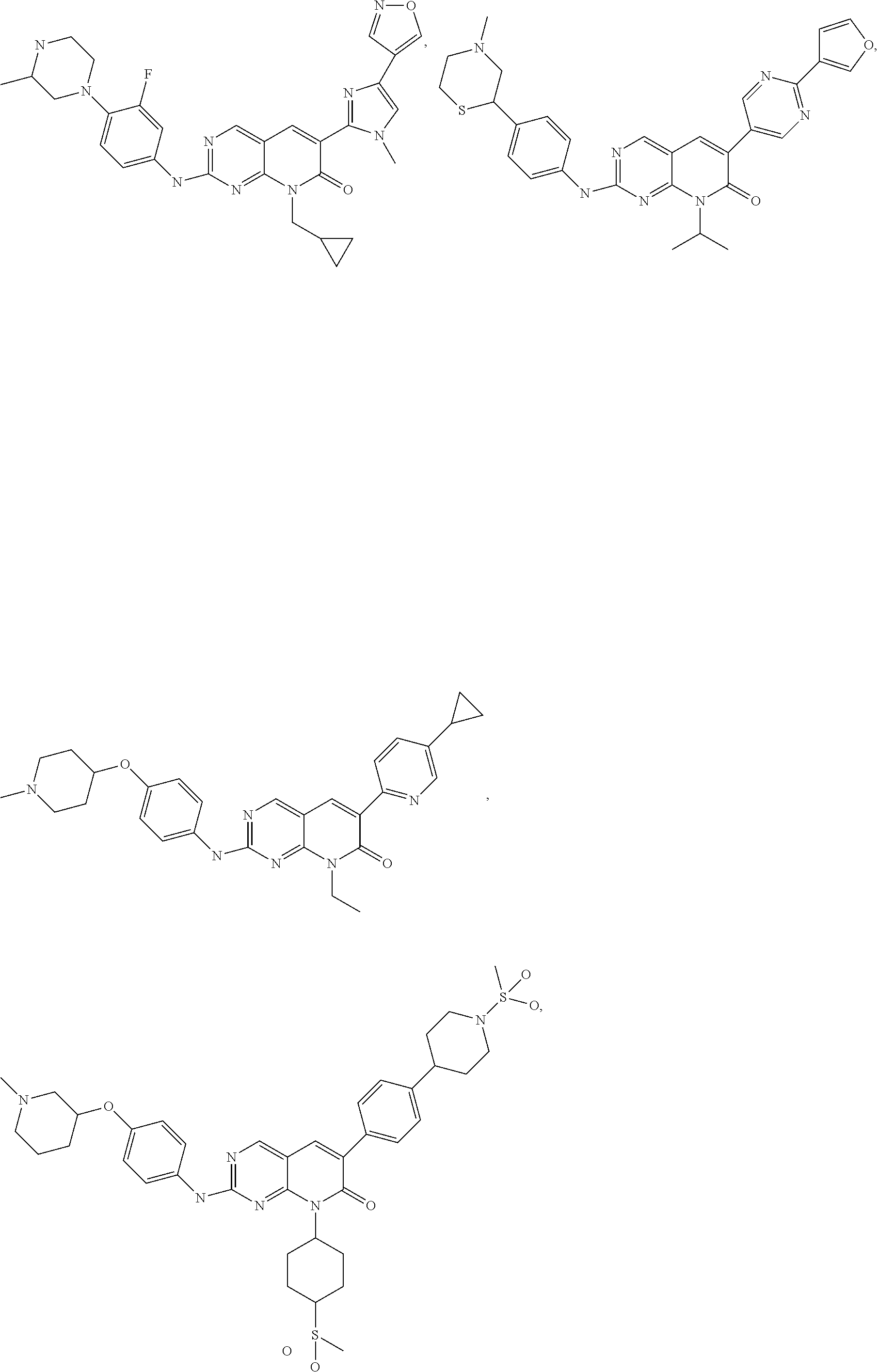

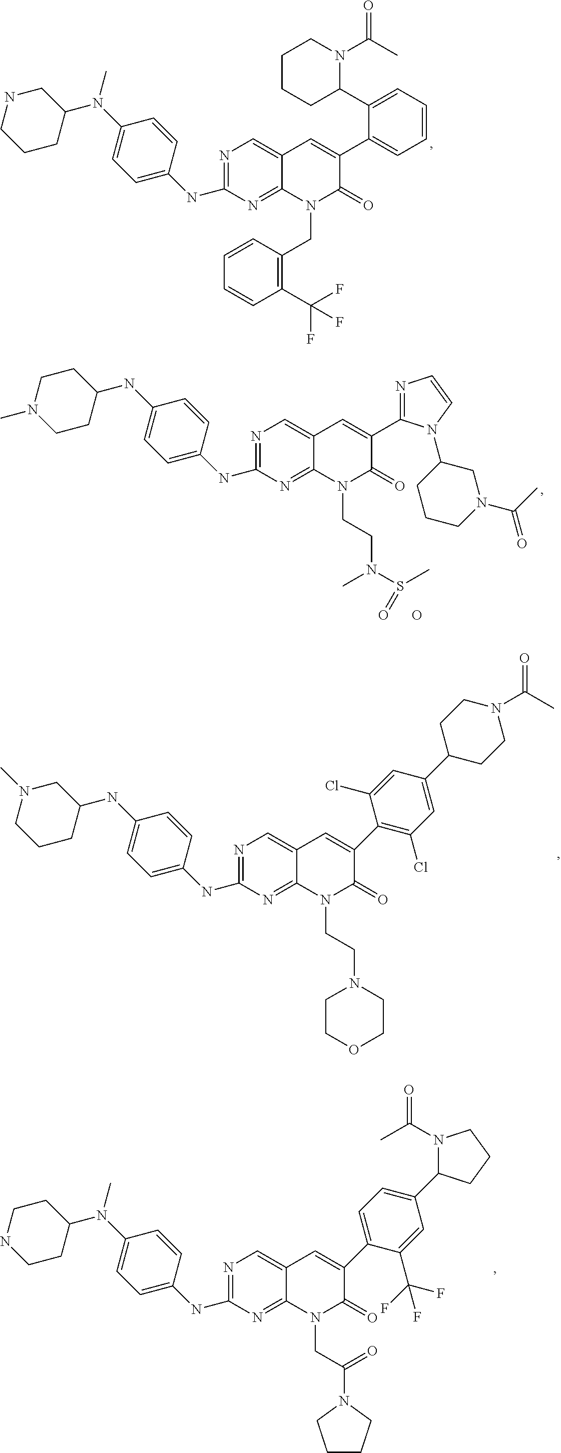

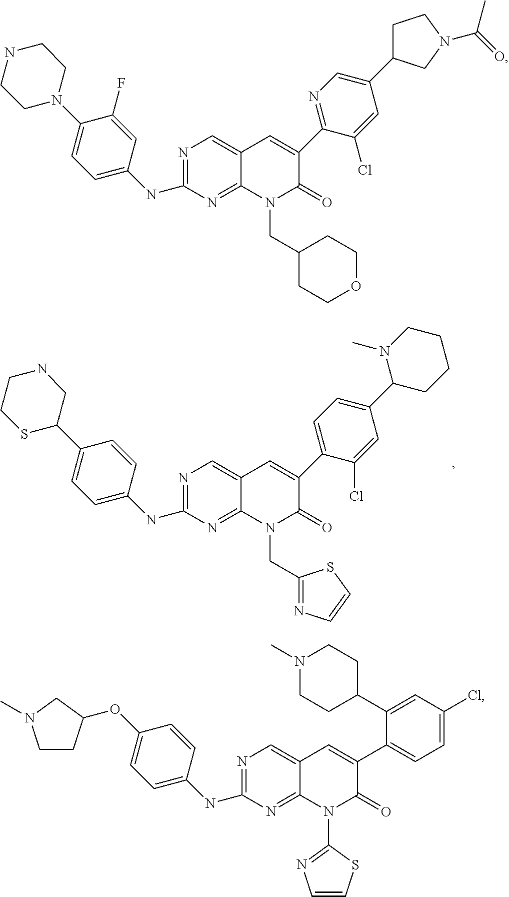

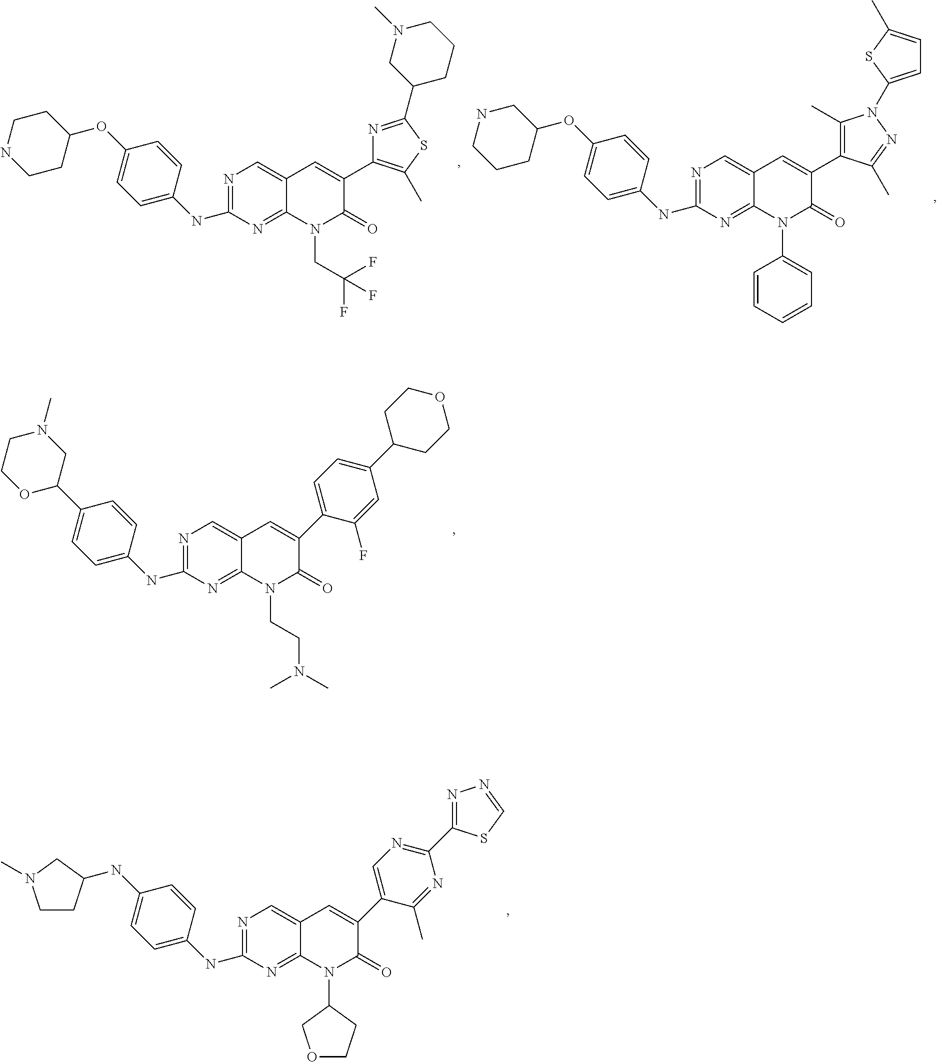

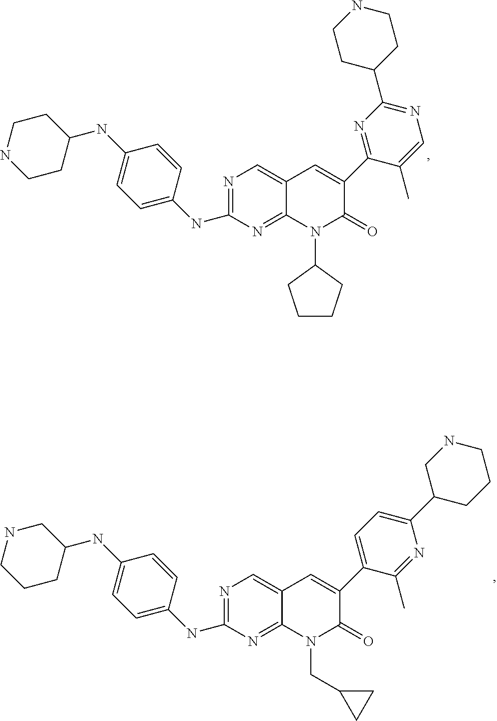 [SMILES: Cc1cnc(C2CCNCC2)nc1-c1cc2cnc(Nc3ccc(NC4CCNCC4)cc3)nc2n(C2CCCC2)c1=O ; Cc1nc(C2CCCNC2)ccc1-c1cc2cnc(Nc3ccc(NC4CCCNC4)cc3)nc2n(CC2CC2)c1=O]

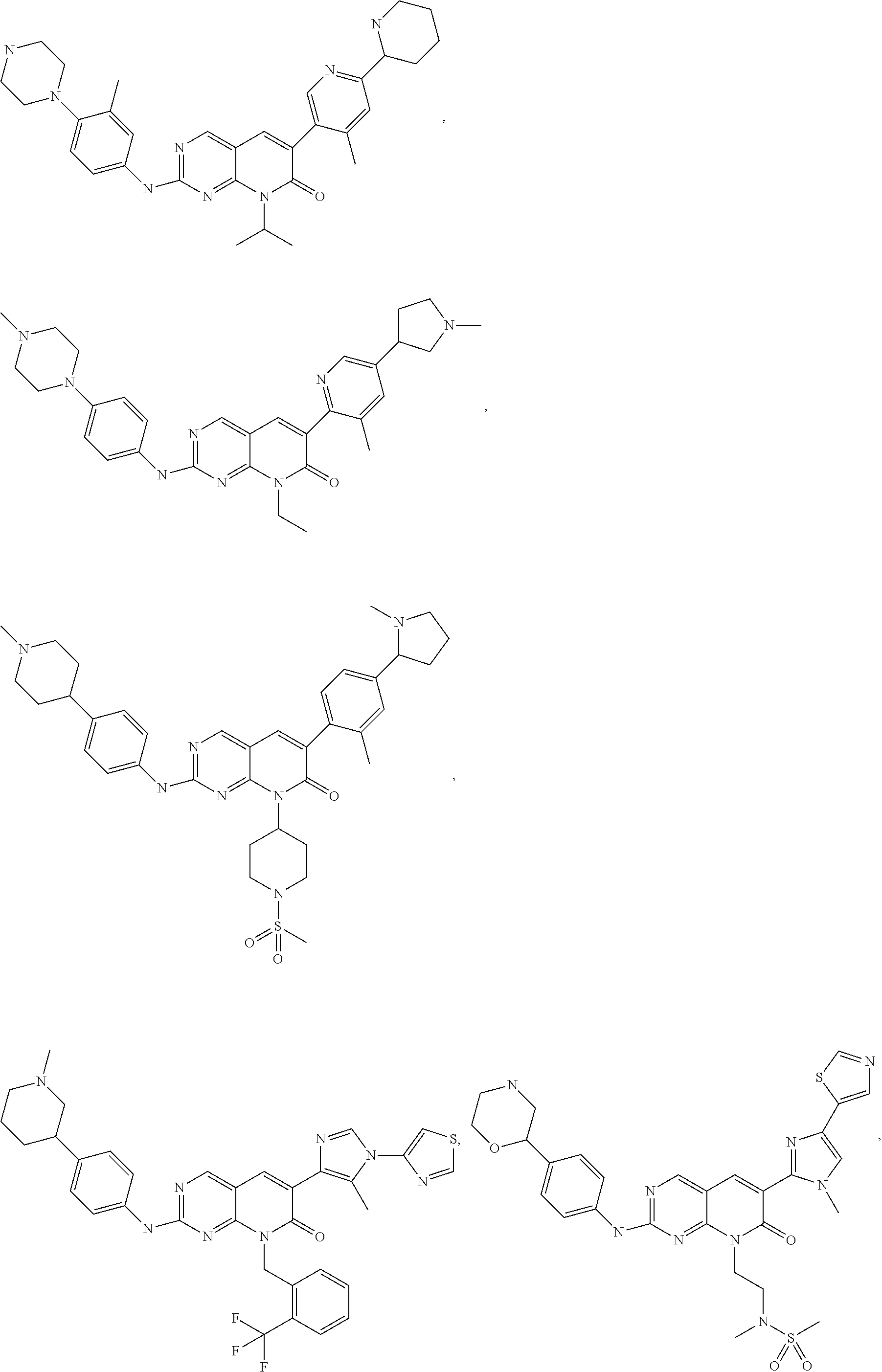

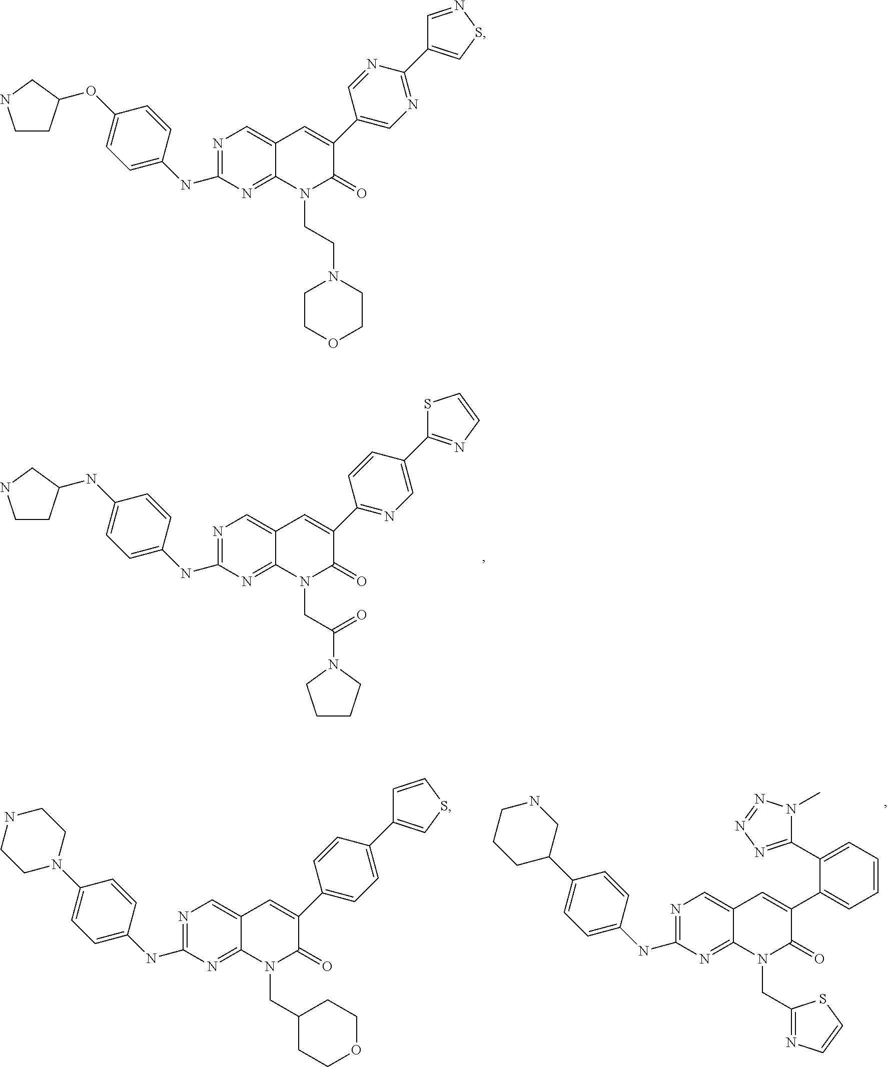 [SMILES: O=c1c(-c2cnc(-c3cnsc3)nc2)cc2cnc(Nc3ccc(OC4CCNC4)cc3)nc2n1CCN1CCOCC1 ; O=C(Cn1c(=O)c(-c2ccc(-c3nccs3)cn2)cc2cnc(Nc3ccc(NC4CCNC4)cc3)nc21)N1CCCC1 ; O=c1c(-c2ccc(-c3ccsc3)cc2)cc2cnc(Nc3ccc(N4CCNCC4)cc3)nc2n1CC1CCOCC1 ; Cn1nnnc1-c1ccccc1-c1cc2cnc(Nc3ccc(C4CCCNC4)cc3)nc2n(Cc2nccs2)c1=O ; ","]

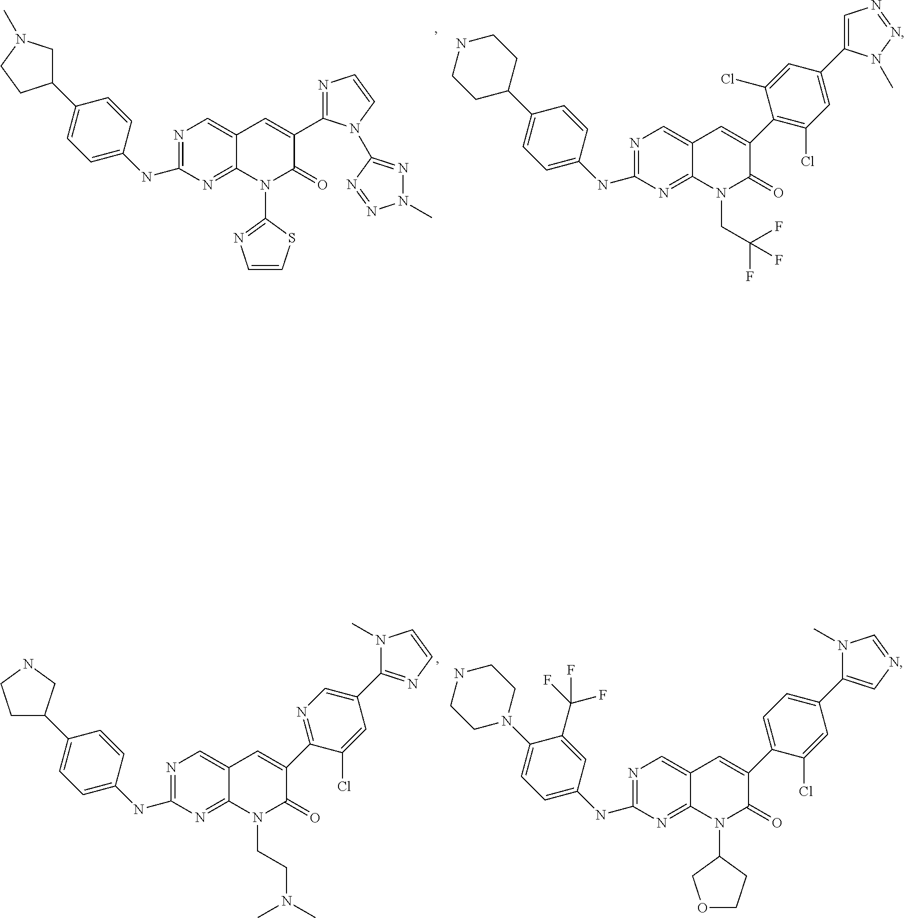

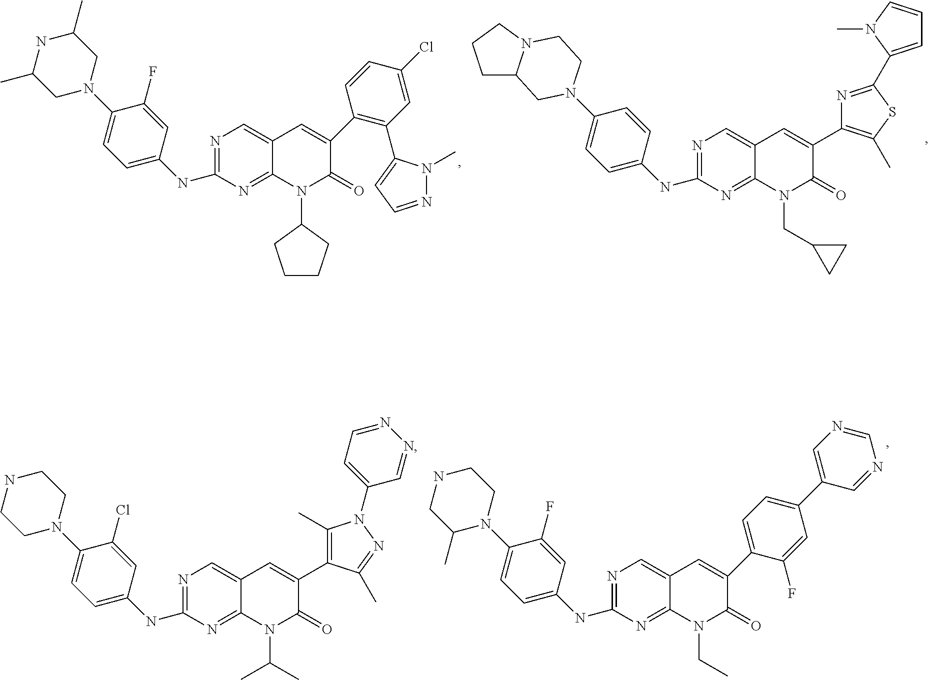

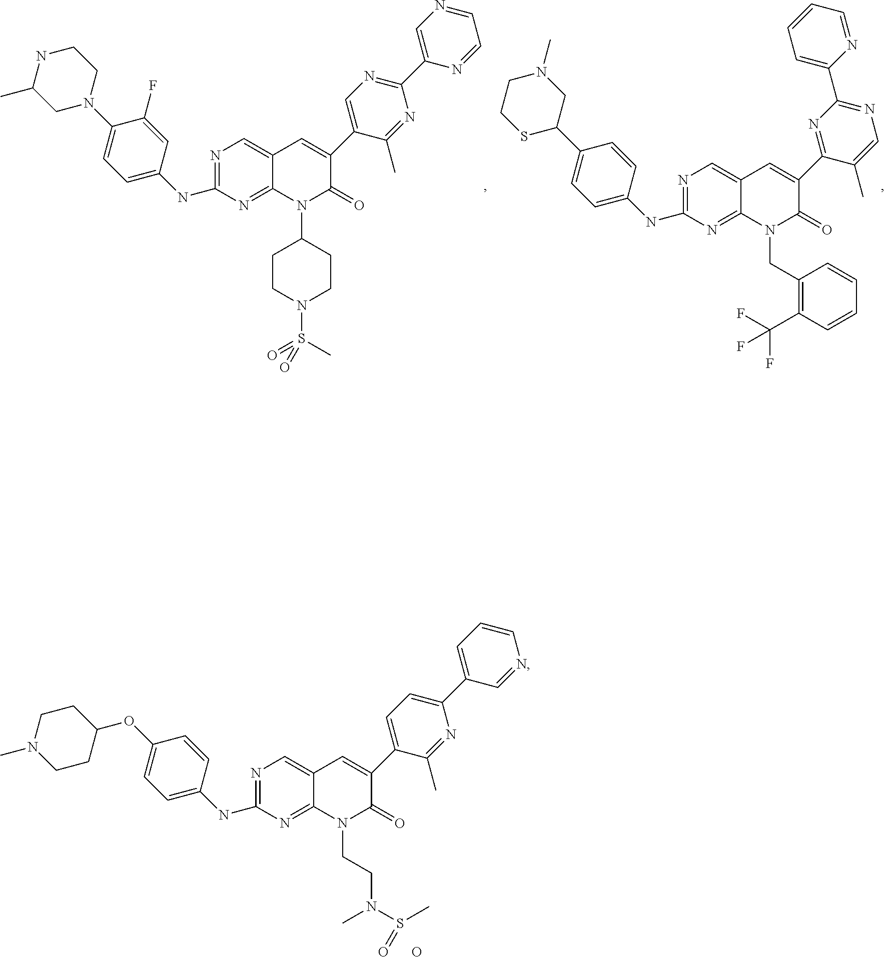

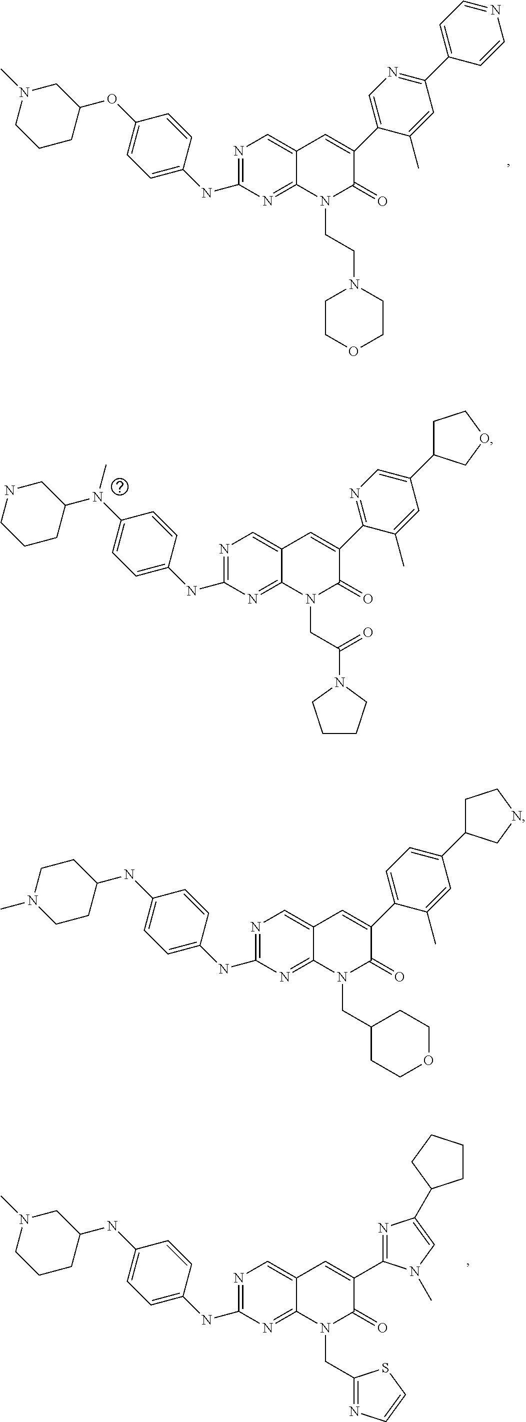

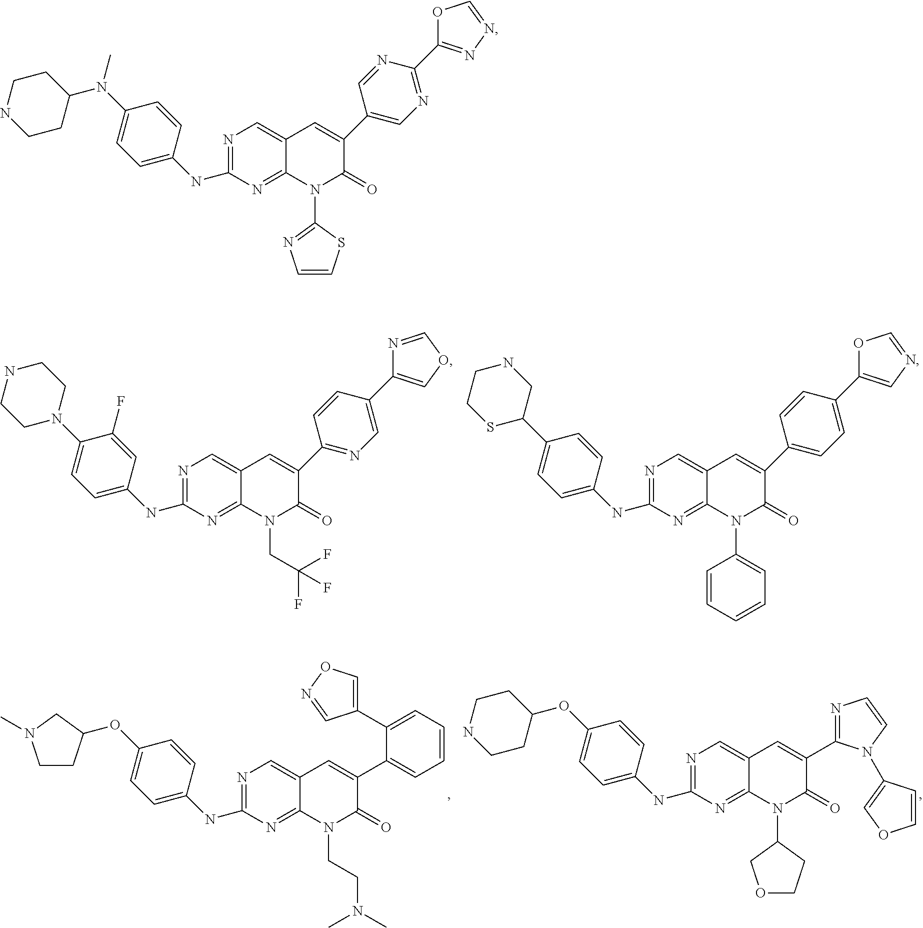

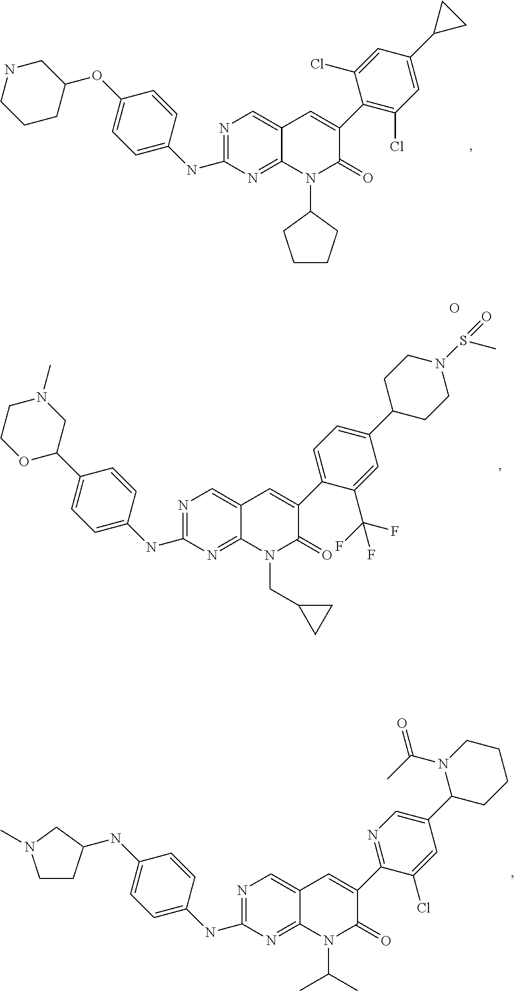

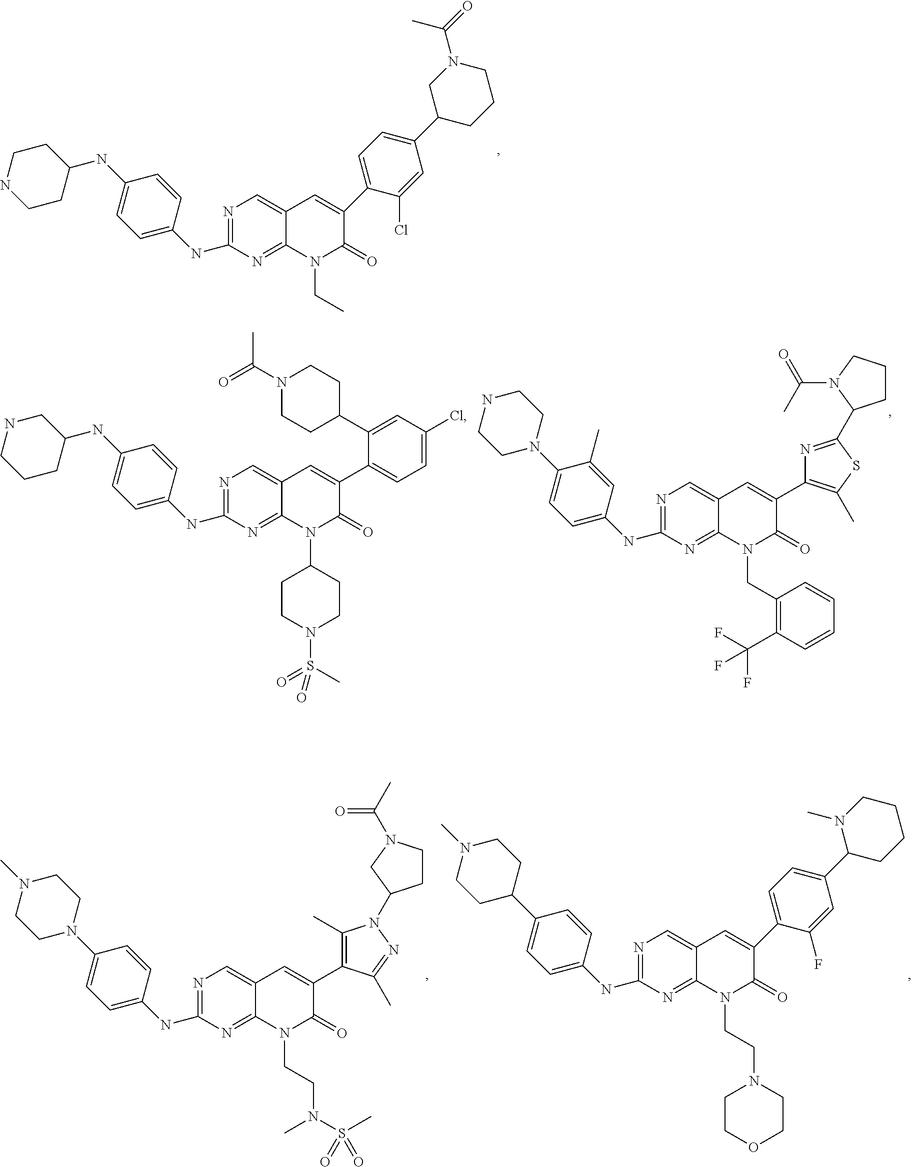

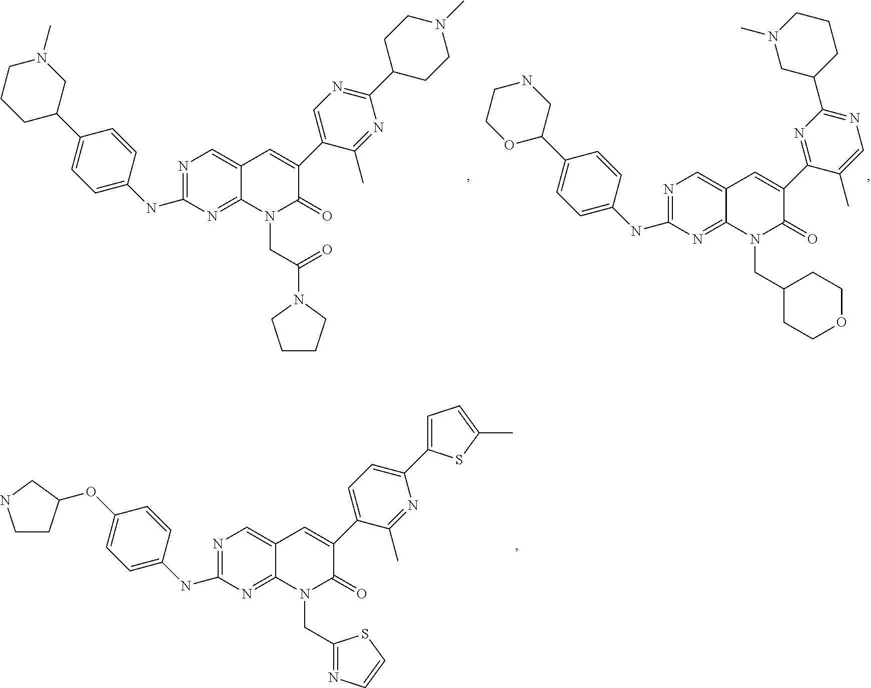 [SMILES: Cc1nc(C2CCN(C)CC2)ncc1-c1cc2cnc(Nc3ccc(C4CCCN(C)C4)cc3)nc2n(CC(=O)N2CCCC2)c1=O ; Cc1cnc(C2CCCN(C)C2)nc1-c1cc2cnc(Nc3ccc(C4CNCCO4)cc3)nc2n(CC2CCOCC2)c1=O ; Cc1ccc(-c2ccc(-c3cc4cnc(Nc5ccc(OC6CCNC6)cc5)nc4n(Cc4nccs4)c3=O)c(C)n2)s1]

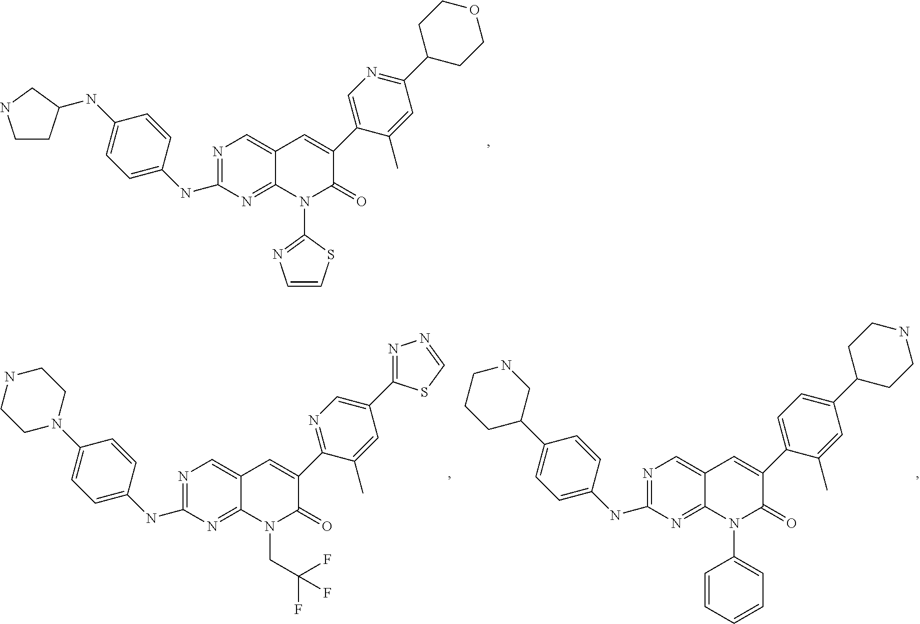

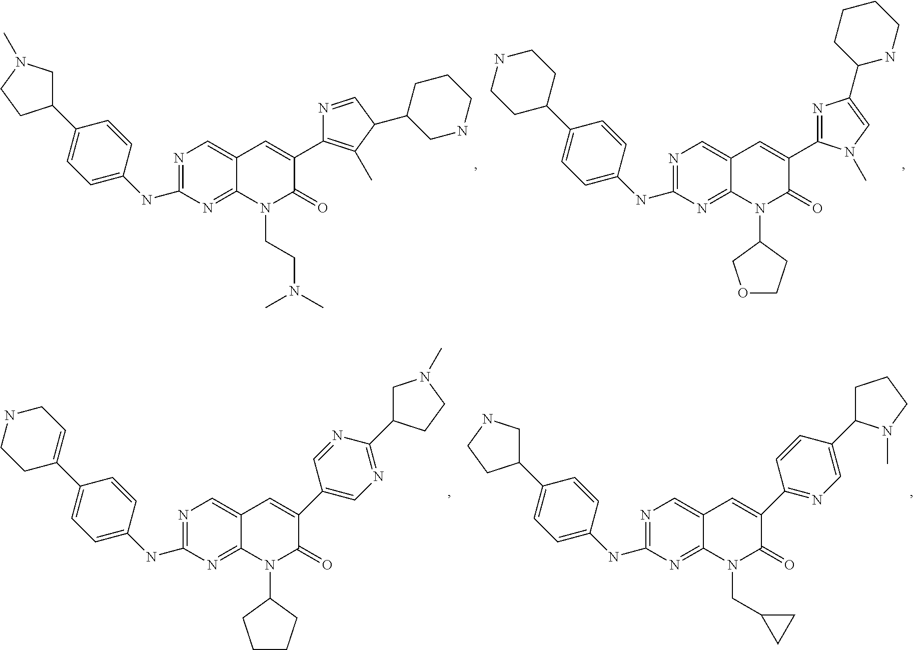

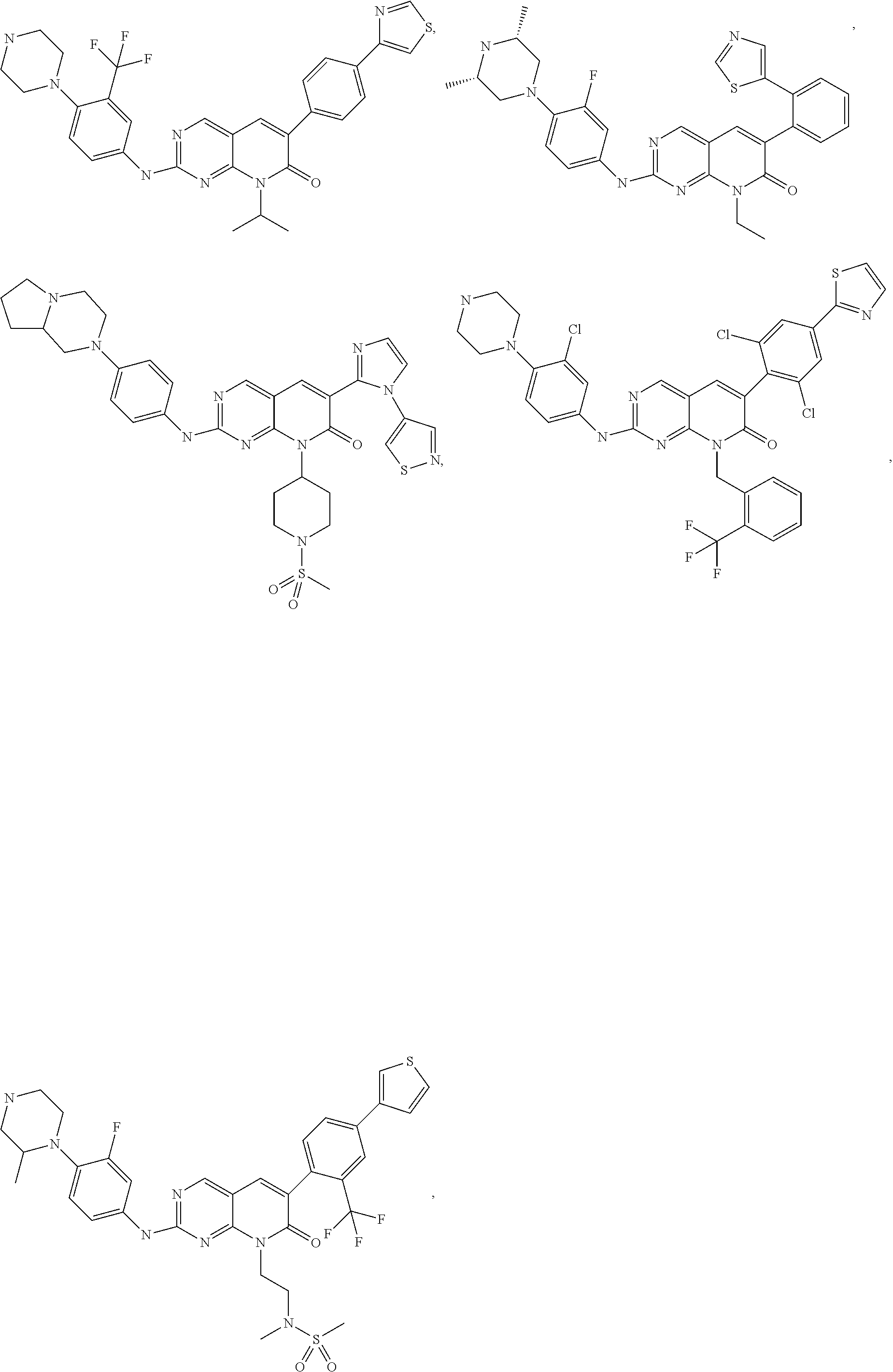

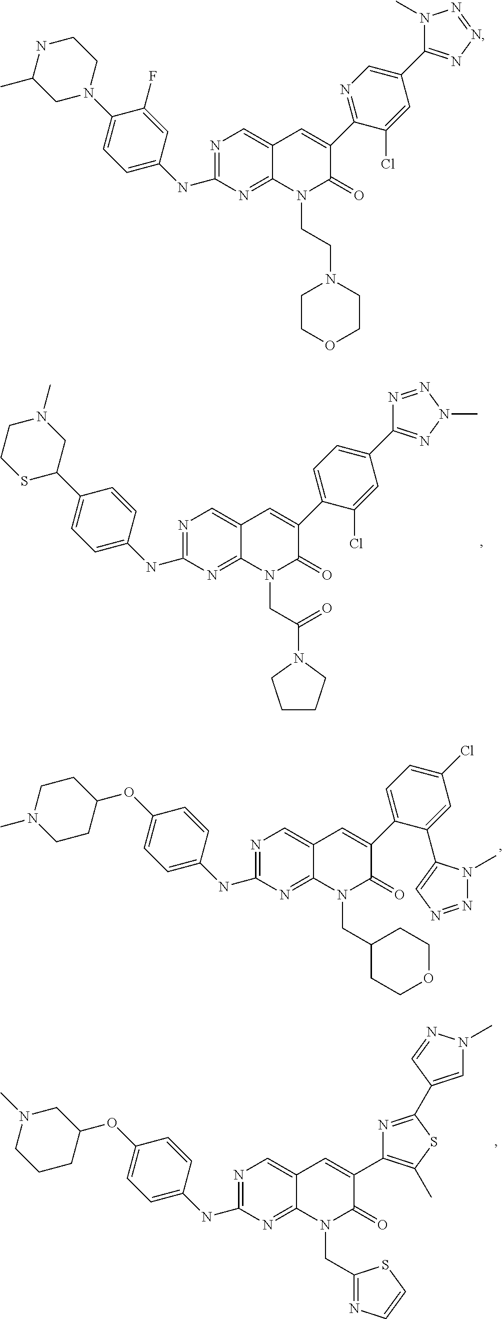

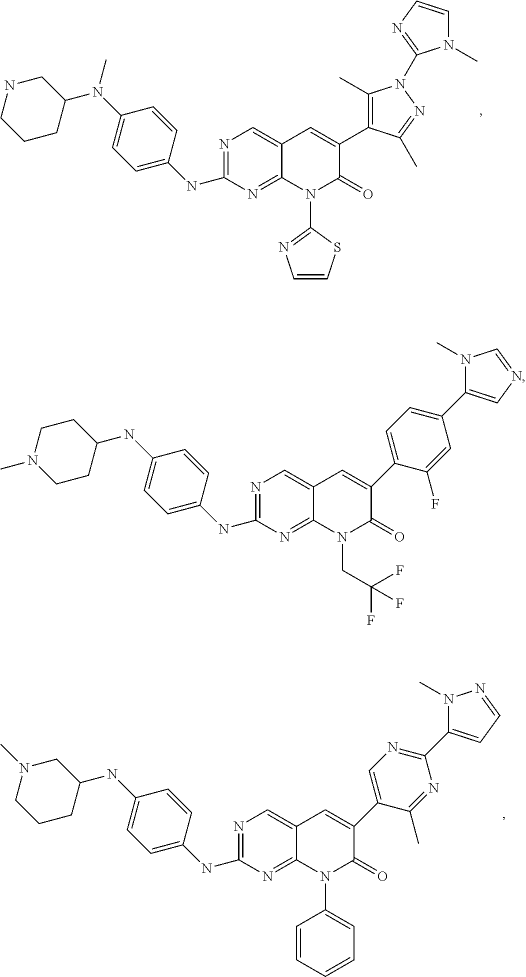

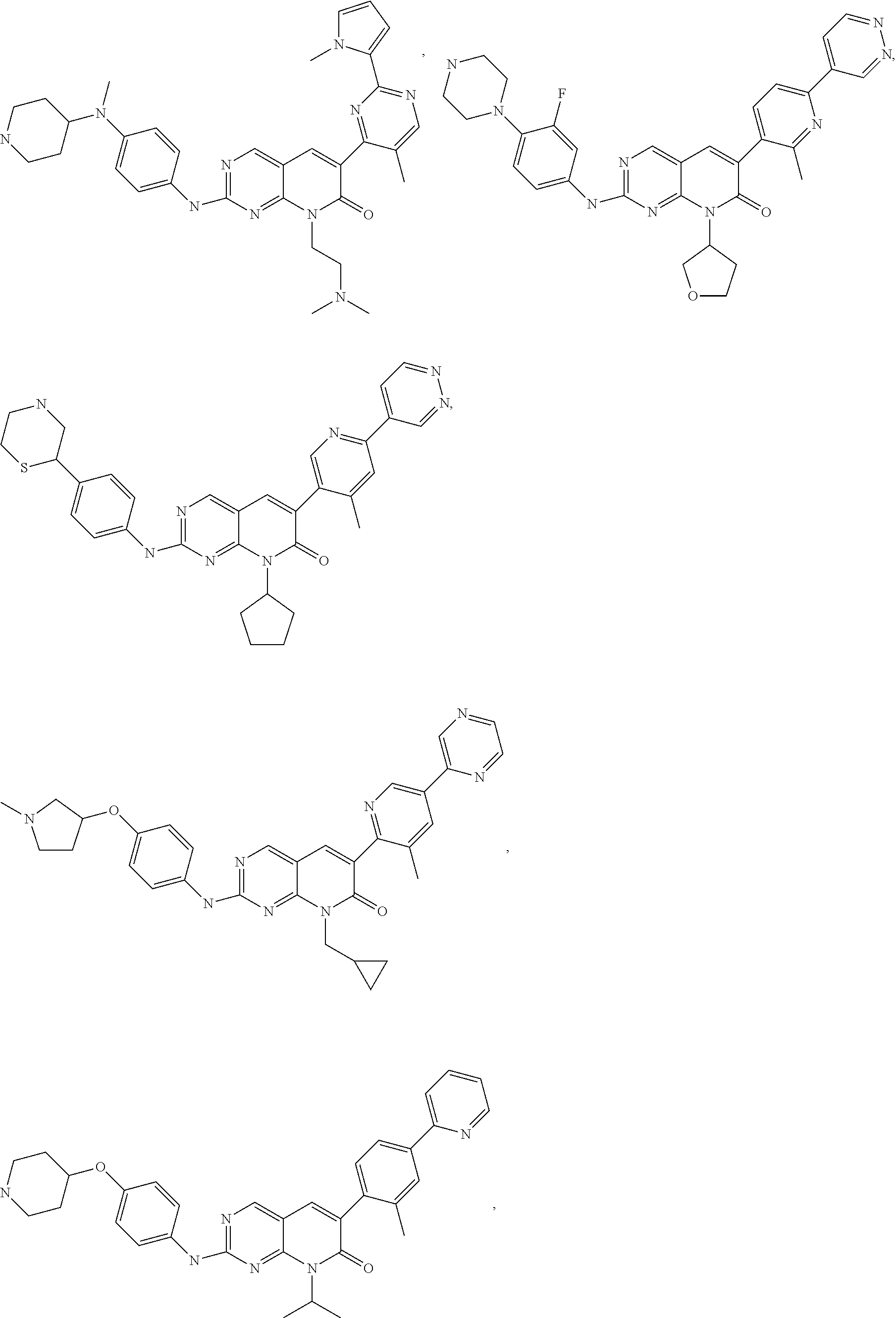

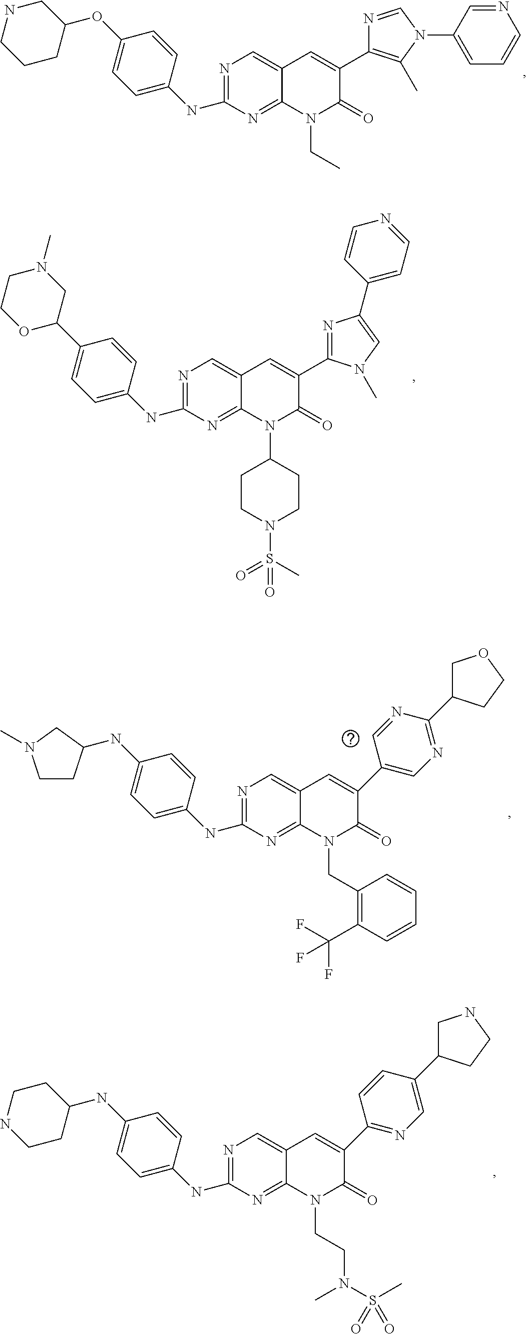 [SMILES: CCn1c(=O)c(-c2ncn(-c3cccnc3)c2C)cc2cnc(Nc3ccc(OC4CCCNC4)cc3)nc21 ; CN1CCOC(c2ccc(Nc3ncc4cc(-c5nc(-c6ccncc6)cn5C)c(=O)n(C5CCN(S(C)(=O)=O)CC5)c4n3)cc2)C1 ; CN1CCC(Nc2ccc(Nc3ncc4cc(-c5cnc(C6CCOC6)nc5)c(=O)n(Cc5ccccc5C(F)(F)F)c4n3)cc2)C1 ; CN(CCn1c(=O)c(-c2ccc(C3CCNC3)cn2)cc2cnc(Nc3ccc(NC4CCNCC4)cc3)nc21)S(C)(=O)=O]

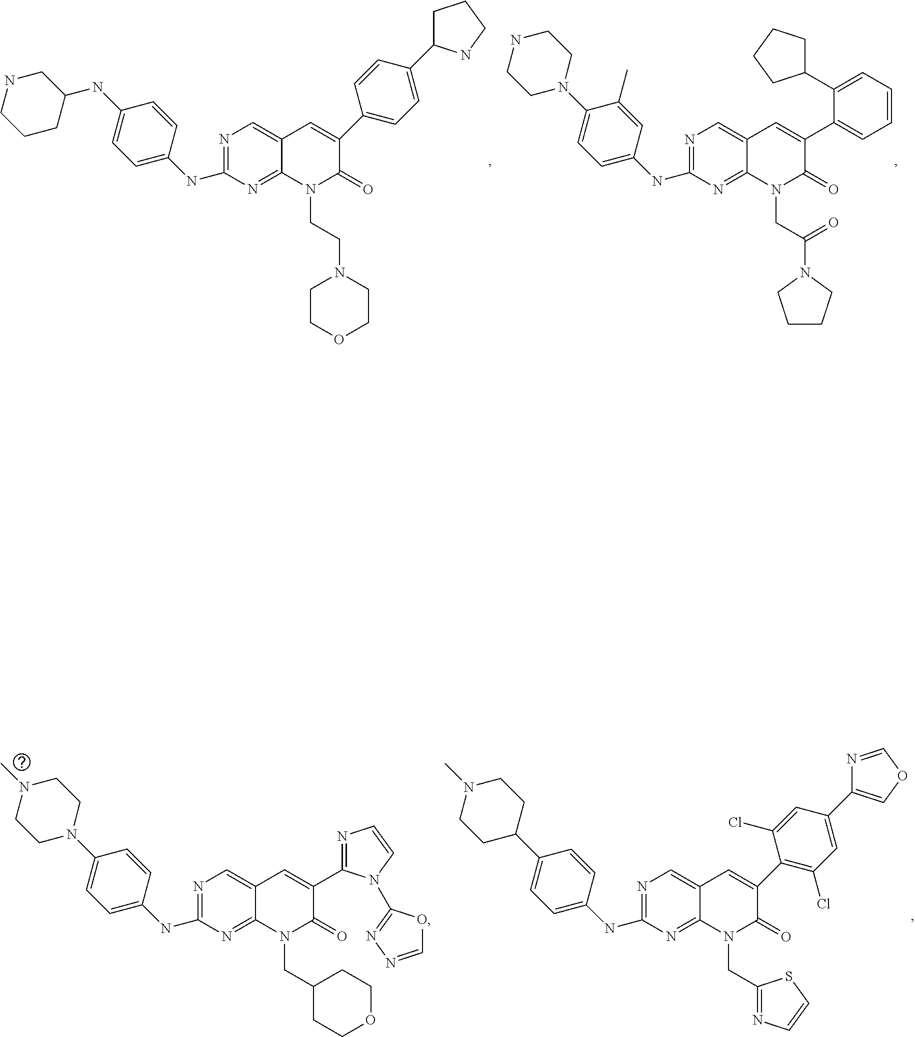

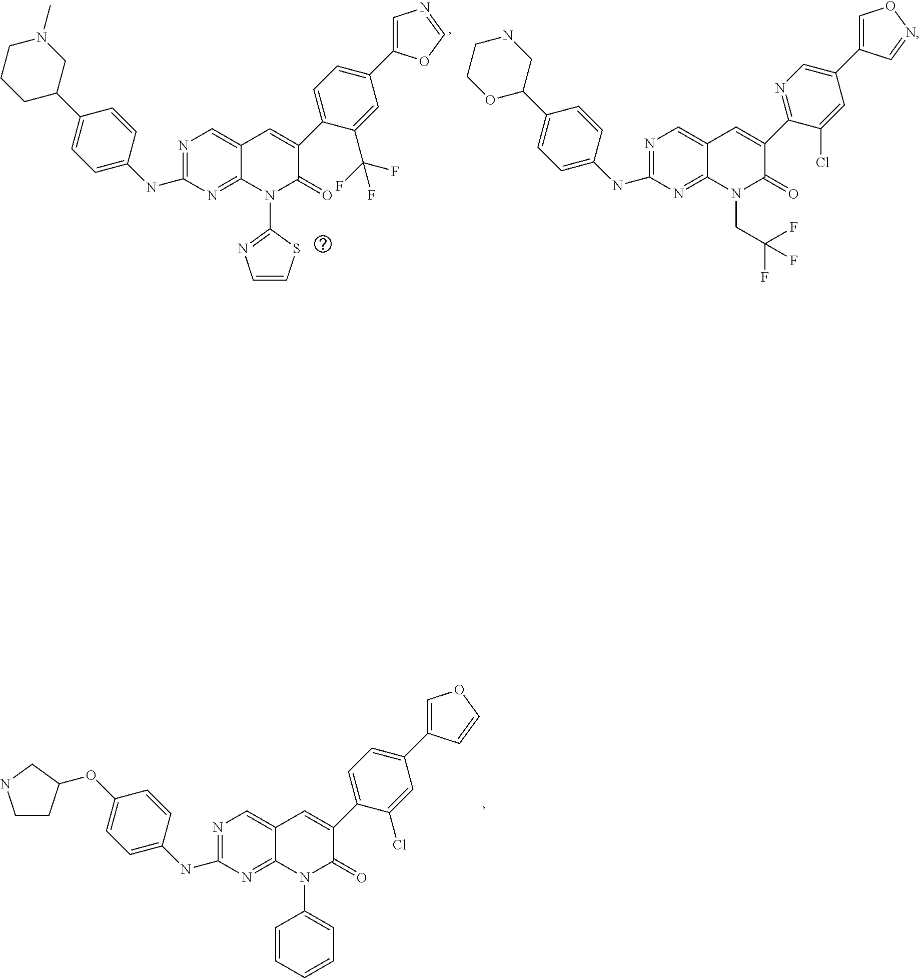

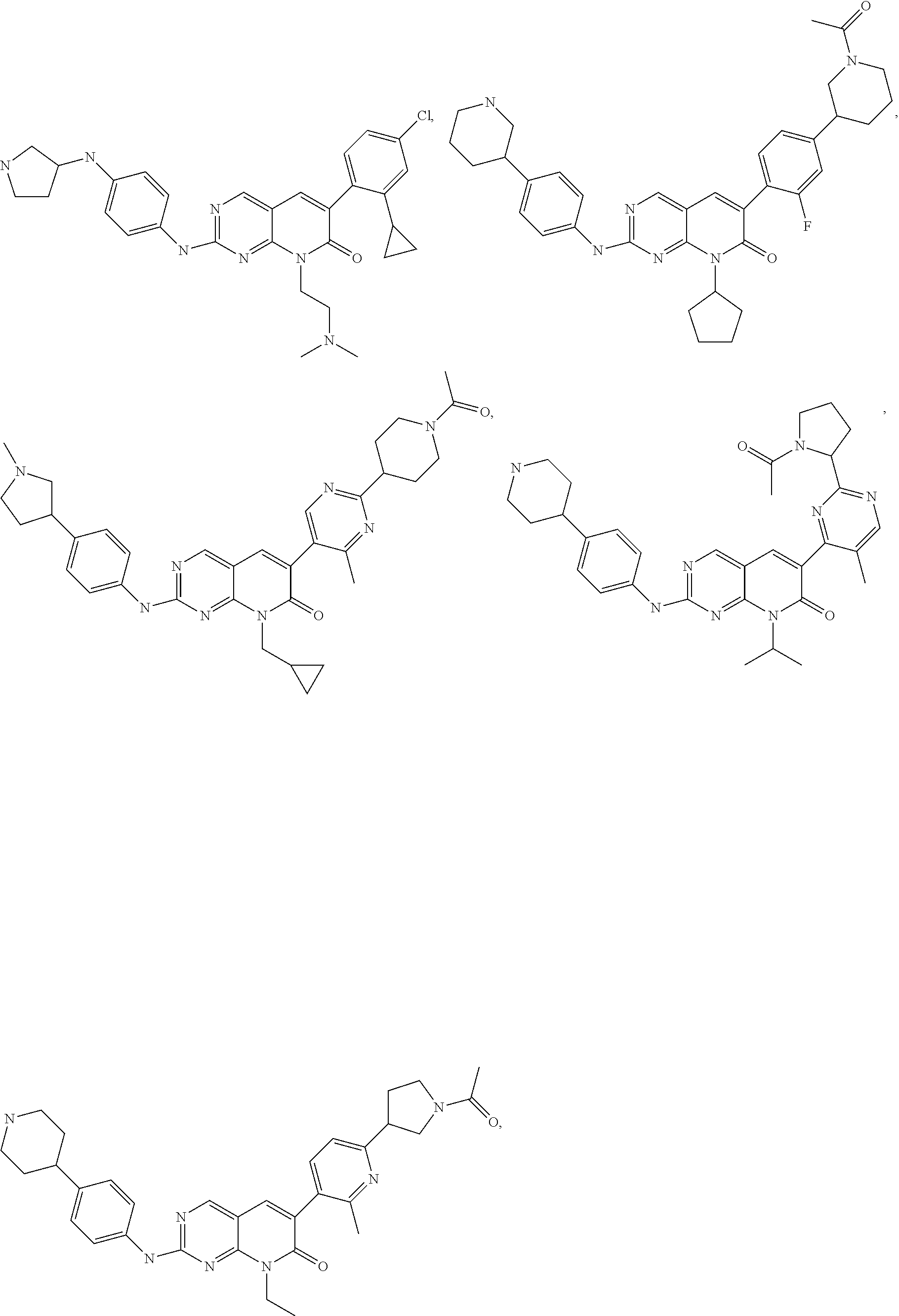

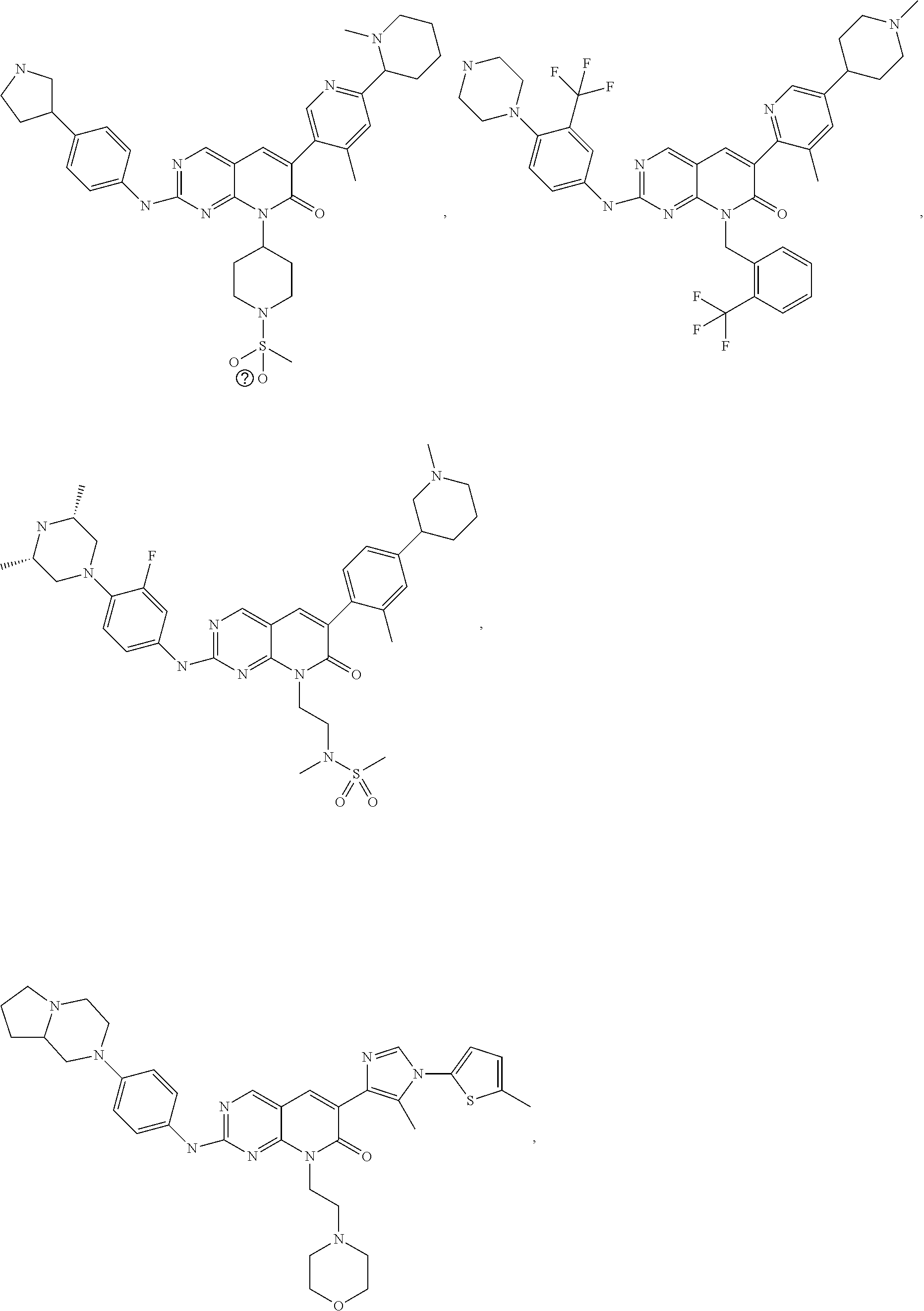

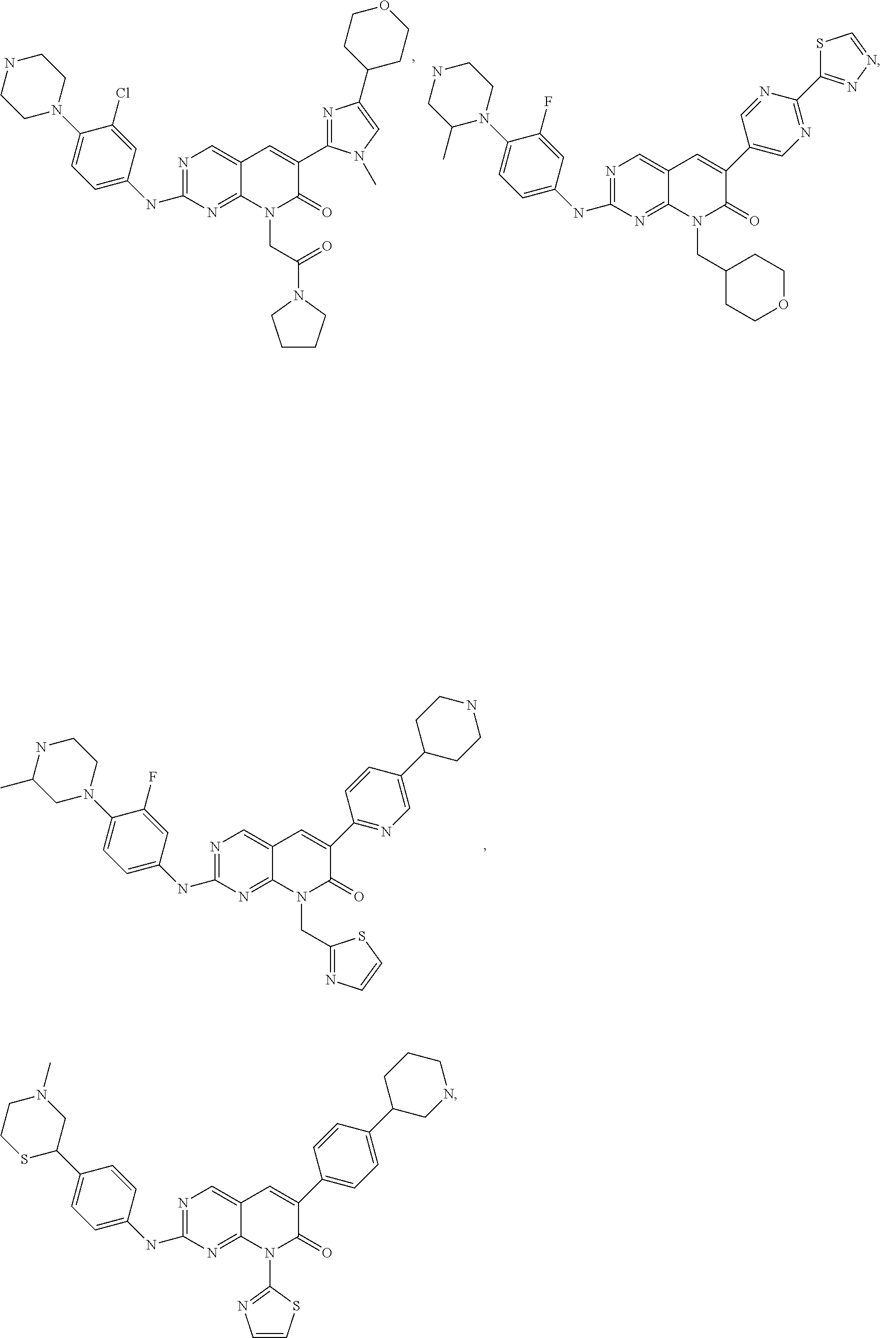

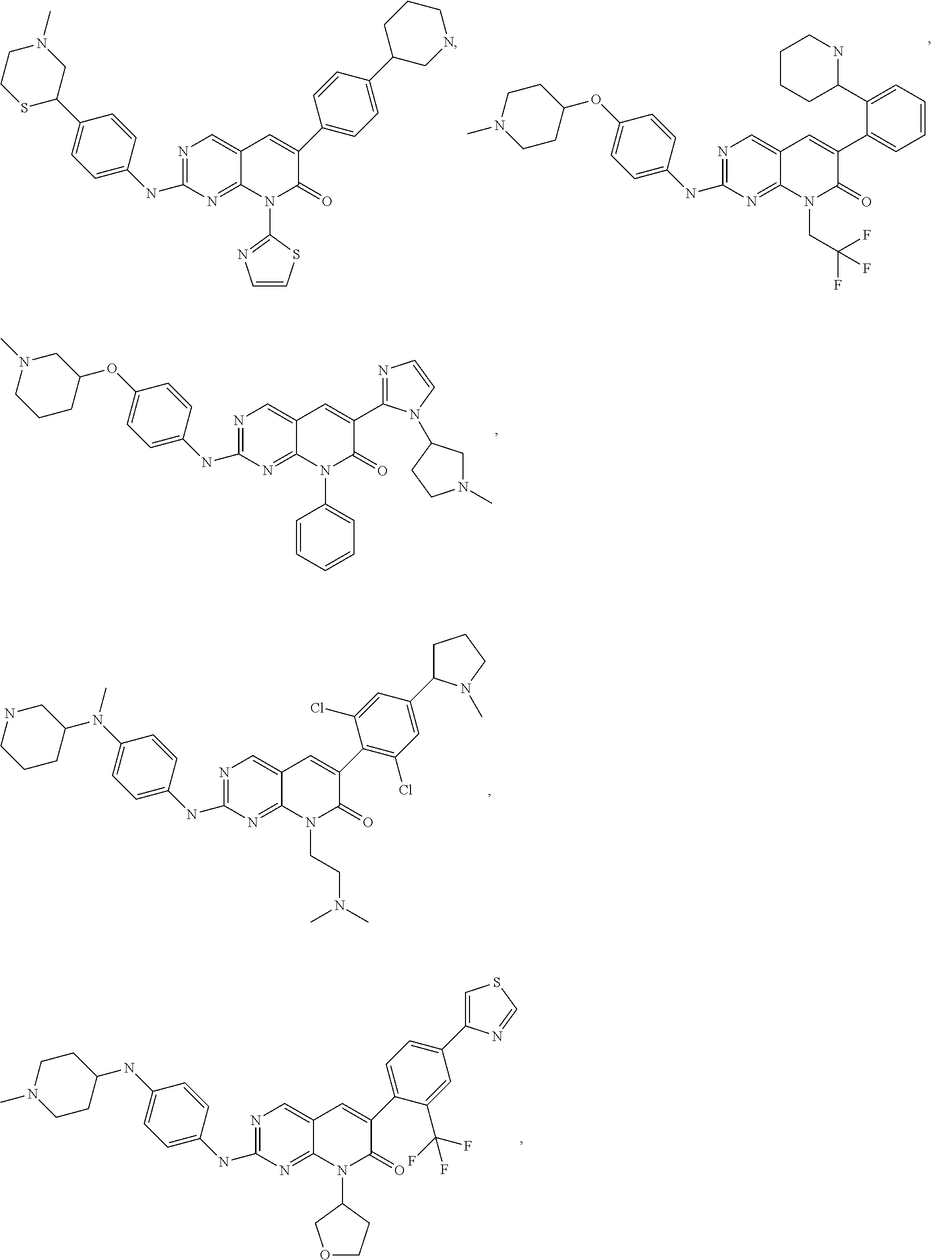

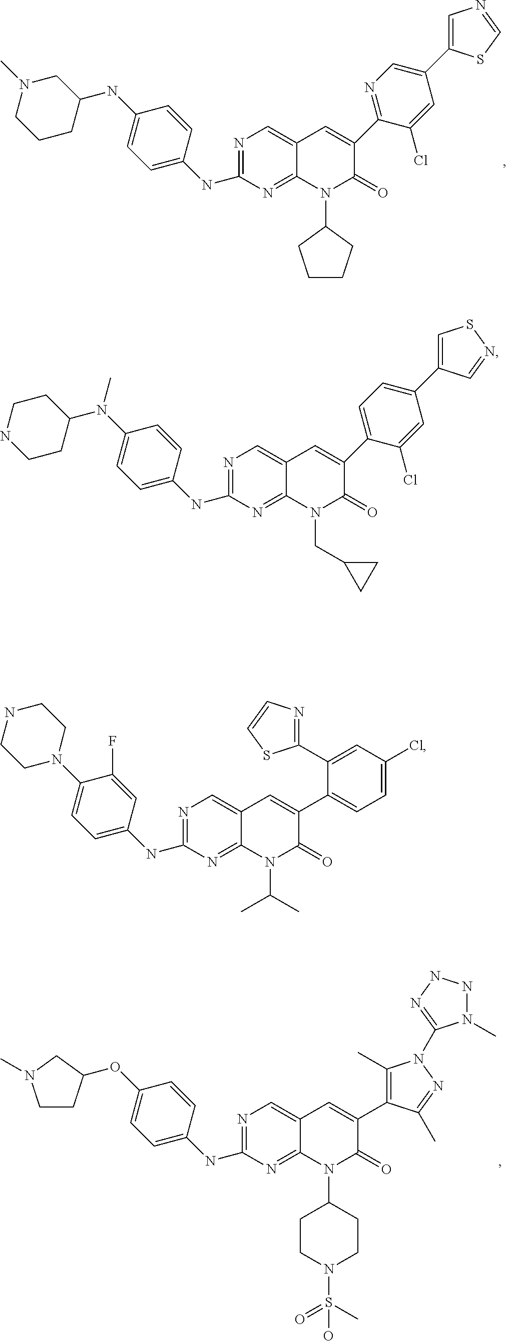

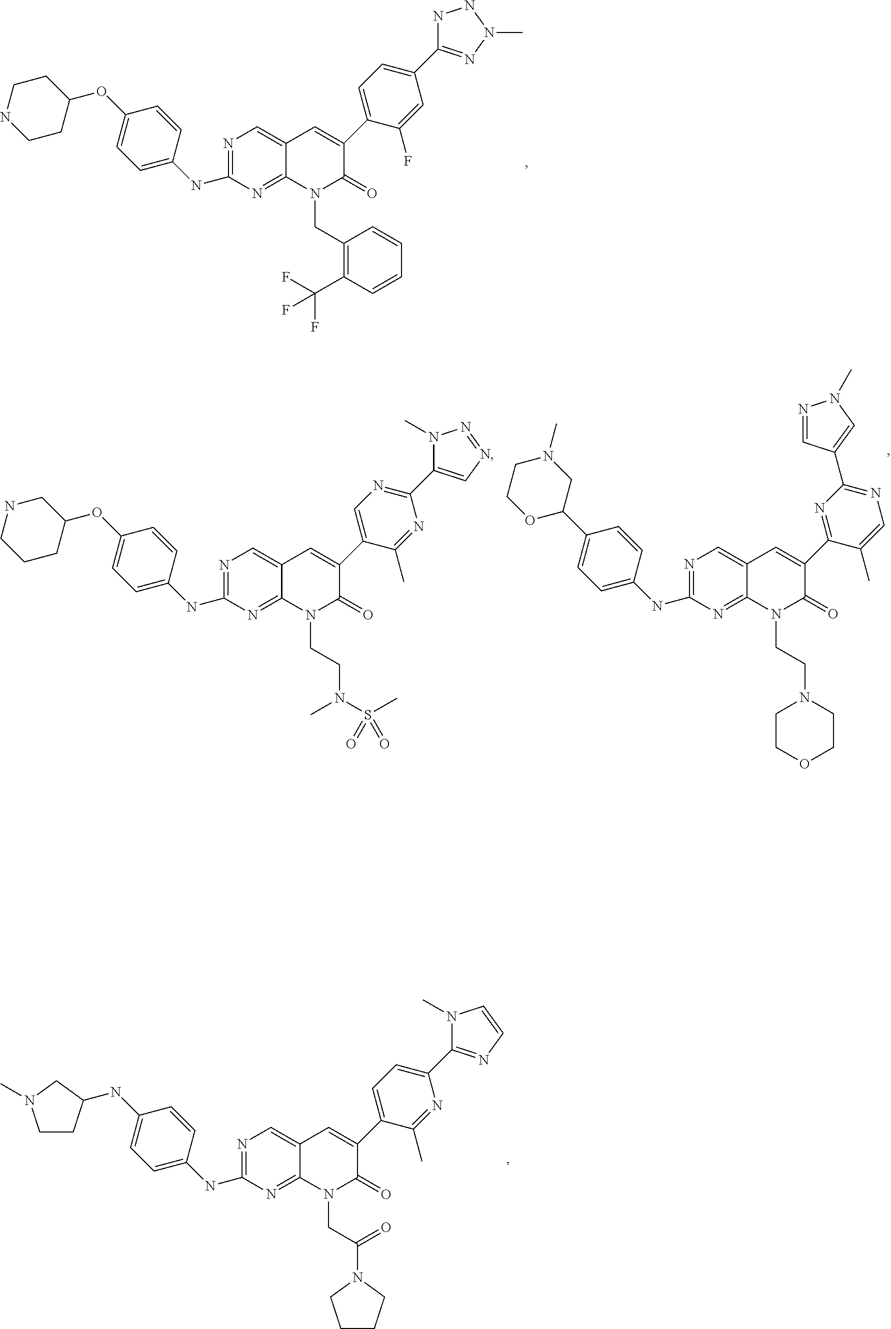

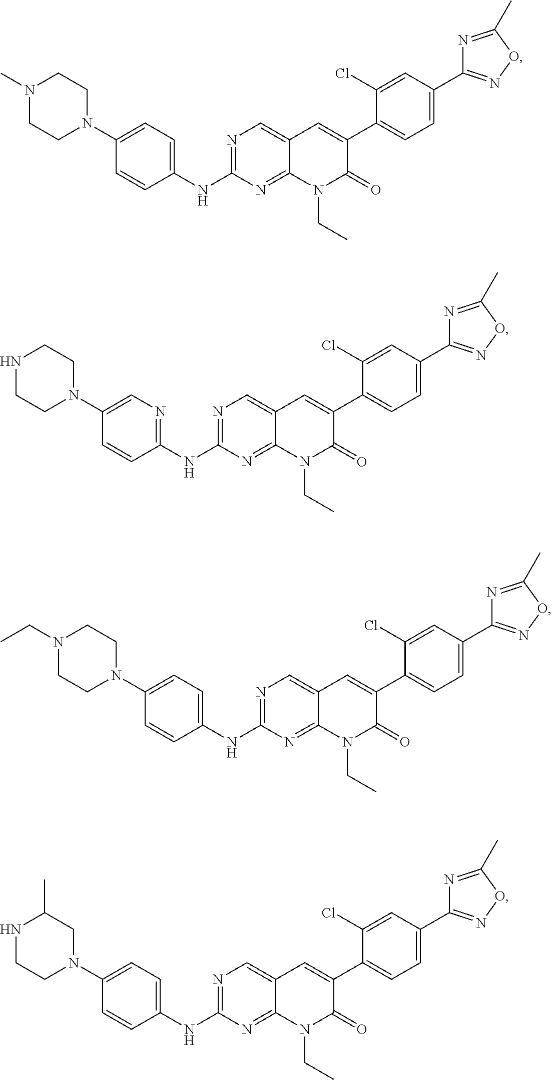 [SMILES: CCn1c(=O)c(-c2ccc(-c3noc(C)n3)cc2Cl)cc2cnc(Nc3ccc(N4CCN(C)CC4)cc3)nc21 ; CCn1c(=O)c(-c2ccc(-c3noc(C)n3)cc2Cl)cc2cnc(Nc3ccc(N4CCNCC4)cn3)nc21 ; CCN1CCN(c2ccc(Nc3ncc4cc(-c5ccc(-c6noc(C)n6)cc5Cl)c(=O)n(CC)c4n3)cc2)CC1 ; CCn1c(=O)c(-c2ccc(-c3noc(C)n3)cc2Cl)cc2cnc(Nc3ccc(N4CCNC(C)C4)cc3)nc21]

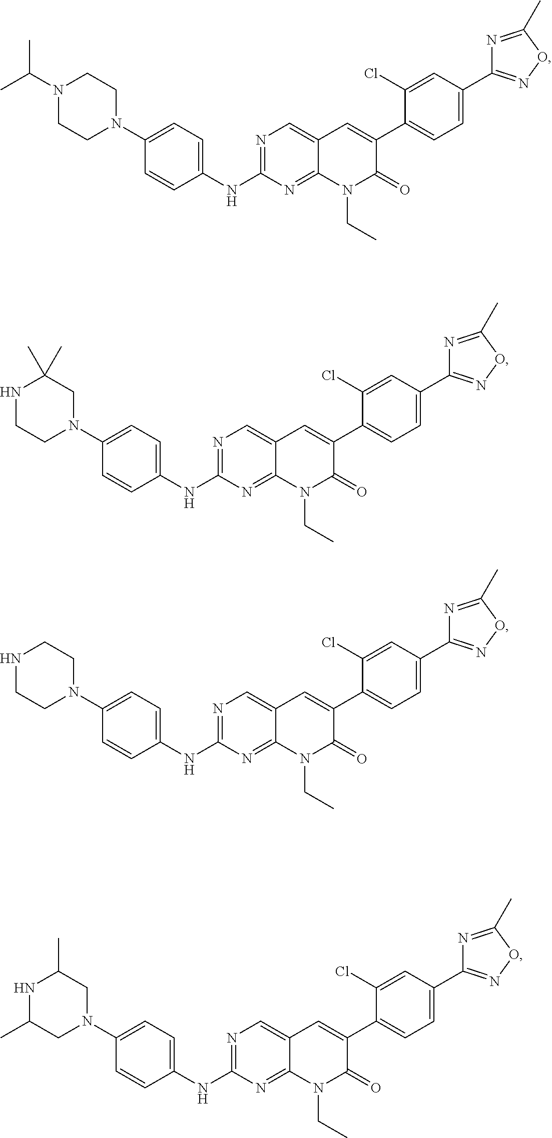 [SMILES: CCn1c(=O)c(-c2ccc(-c3noc(C)n3)cc2Cl)cc2cnc(Nc3ccc(N4CCN(C(C)C)CC4)cc3)nc21 ; CCn1c(=O)c(-c2ccc(-c3noc(C)n3)cc2Cl)cc2cnc(Nc3ccc(N4CCNC(C)(C)C4)cc3)nc21 ; CCn1c(=O)c(-c2ccc(-c3noc(C)n3)cc2Cl)cc2cnc(Nc3ccc(N4CCNCC4)cc3)nc21 ; CCn1c(=O)c(-c2ccc(-c3noc(C)n3)cc2Cl)cc2cnc(Nc3ccc(N4CC(C)NC(C)C4)cc3)nc21]

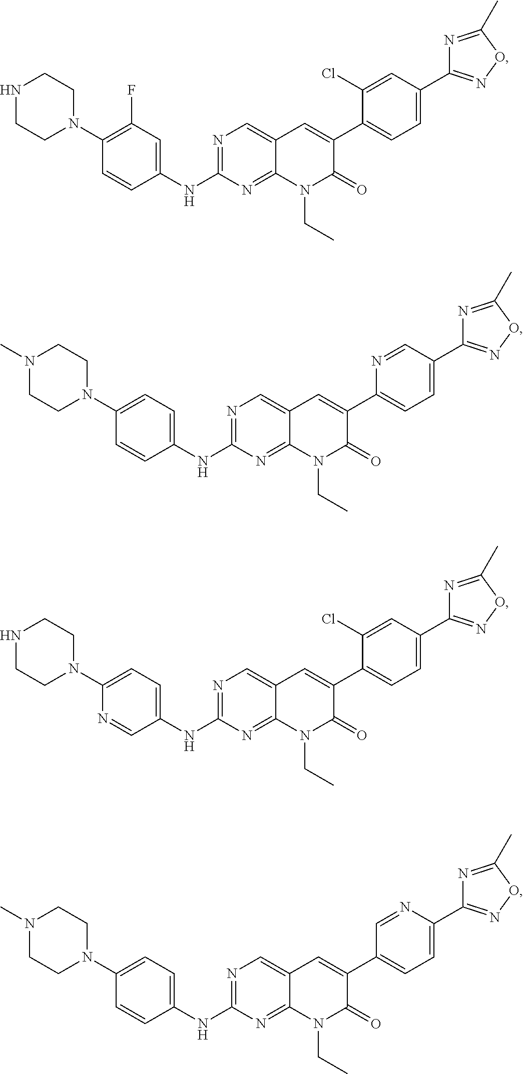 [SMILES: CCn1c(=O)c(-c2ccc(-c3noc(C)n3)cc2Cl)cc2cnc(Nc3ccc(N4CCNCC4)c(F)c3)nc21 ; CCn1c(=O)c(-c2ccc(-c3noc(C)n3)cn2)cc2cnc(Nc3ccc(N4CCN(C)CC4)cc3)nc21 ; CCn1c(=O)c(-c2ccc(-c3noc(C)n3)cc2Cl)cc2cnc(Nc3ccc(N4CCNCC4)nc3)nc21 ; CCn1c(=O)c(-c2ccc(-c3noc(C)n3)nc2)cc2cnc(Nc3ccc(N4CCN(C)CC4)cc3)nc21]

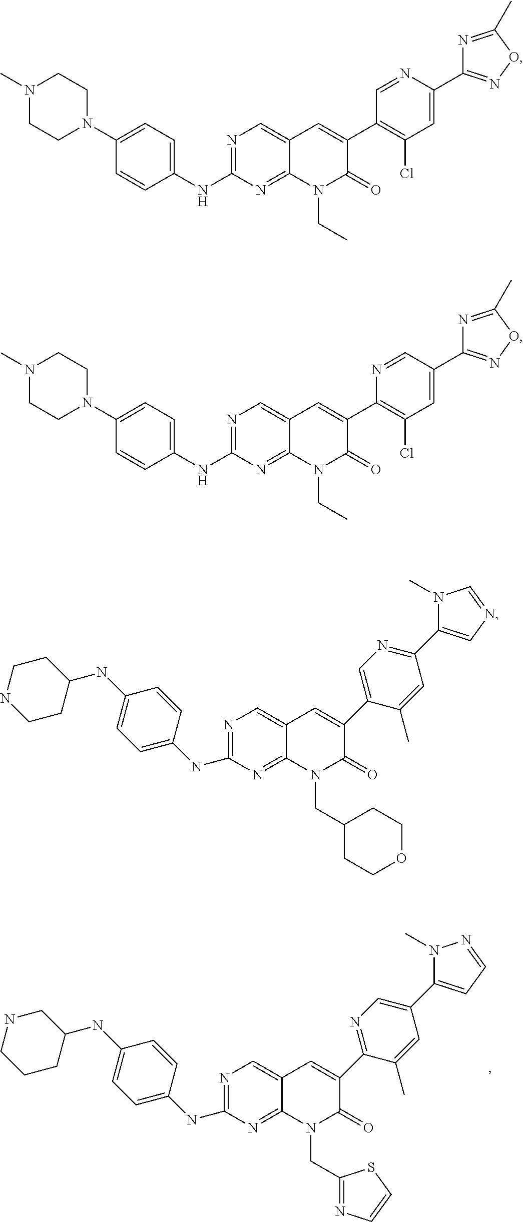 [SMILES: CCn1c(=O)c(-c2cnc(-c3noc(C)n3)cc2Cl)cc2cnc(Nc3ccc(N4CCN(C)CC4)cc3)nc21 ; CCn1c(=O)c(-c2ncc(-c3noc(C)n3)cc2Cl)cc2cnc(Nc3ccc(N4CCN(C)CC4)cc3)nc21 ; Cc1cc(-c2cncn2C)ncc1-c1cc2cnc(Nc3ccc(NC4CCNCC4)cc3)nc2n(CC2CCOCC2)c1=O ; Cc1cc(-c2ccnn2C)cnc1-c1cc2cnc(Nc3ccc(NC4CCCNC4)cc3)nc2n(Cc2nccs2)c1=O]

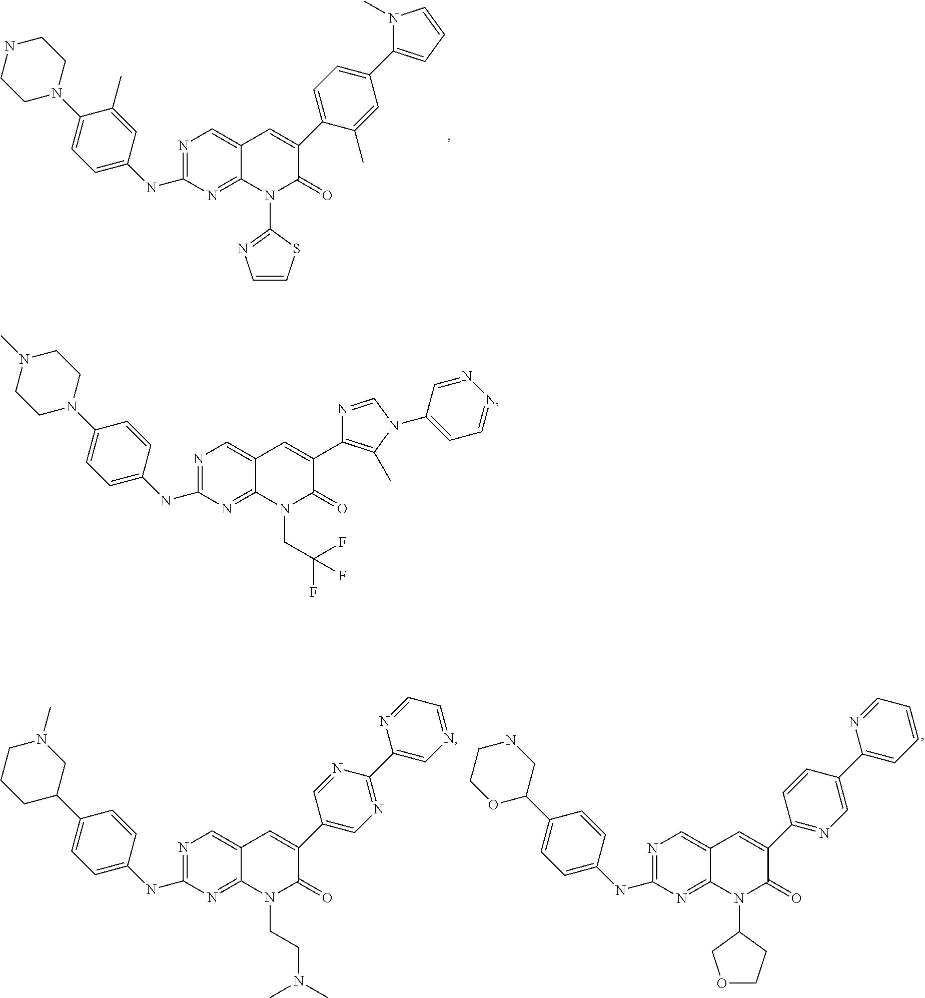

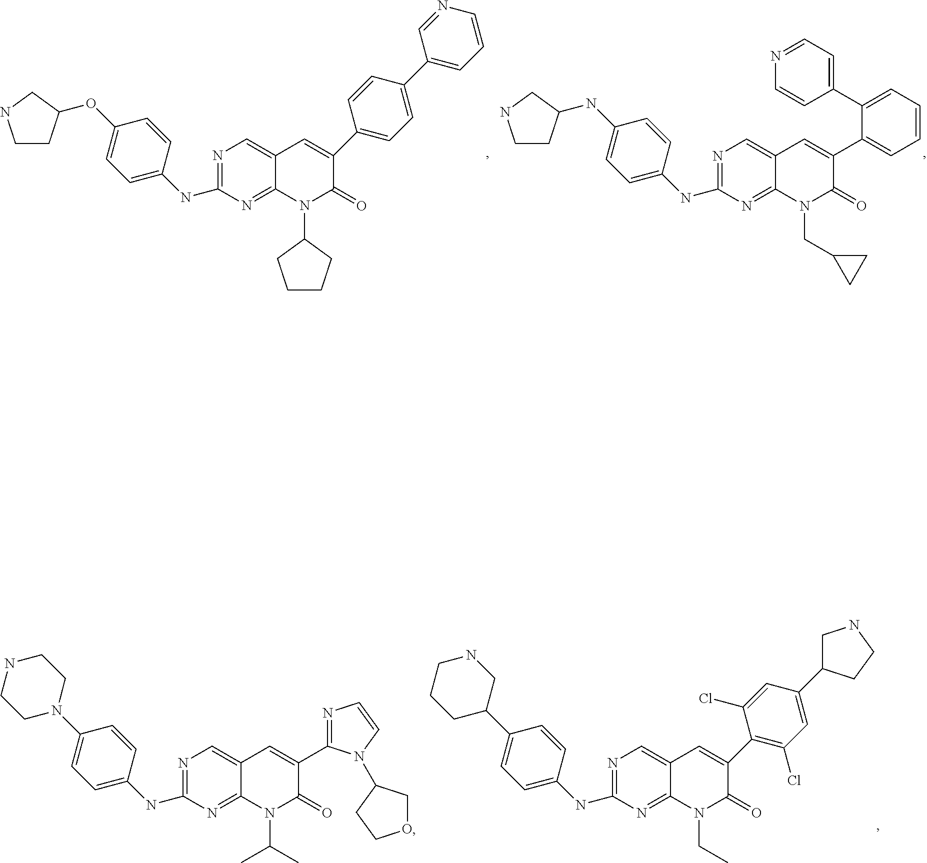 [SMILES: O=c1c(-c2ccc(-c3cccnc3)cc2)cc2cnc(Nc3ccc(OC4CCNC4)cc3)nc2n1C1CCCC1 ; O=c1c(-c2ccccc2-c2ccncc2)cc2cnc(Nc3ccc(NC4CCNC4)cc3)nc2n1CC1CC1 ; CC(C)n1c(=O)c(-c2nccn2C2CCOC2)cc2cnc(Nc3ccc(N4CCNCC4)cc3)nc21 ; CCn1c(=O)c(-c2c(Cl)cc(C3CCNC3)cc2Cl)cc2cnc(Nc3ccc(C4CCCNC4)cc3)nc21 ; ","]

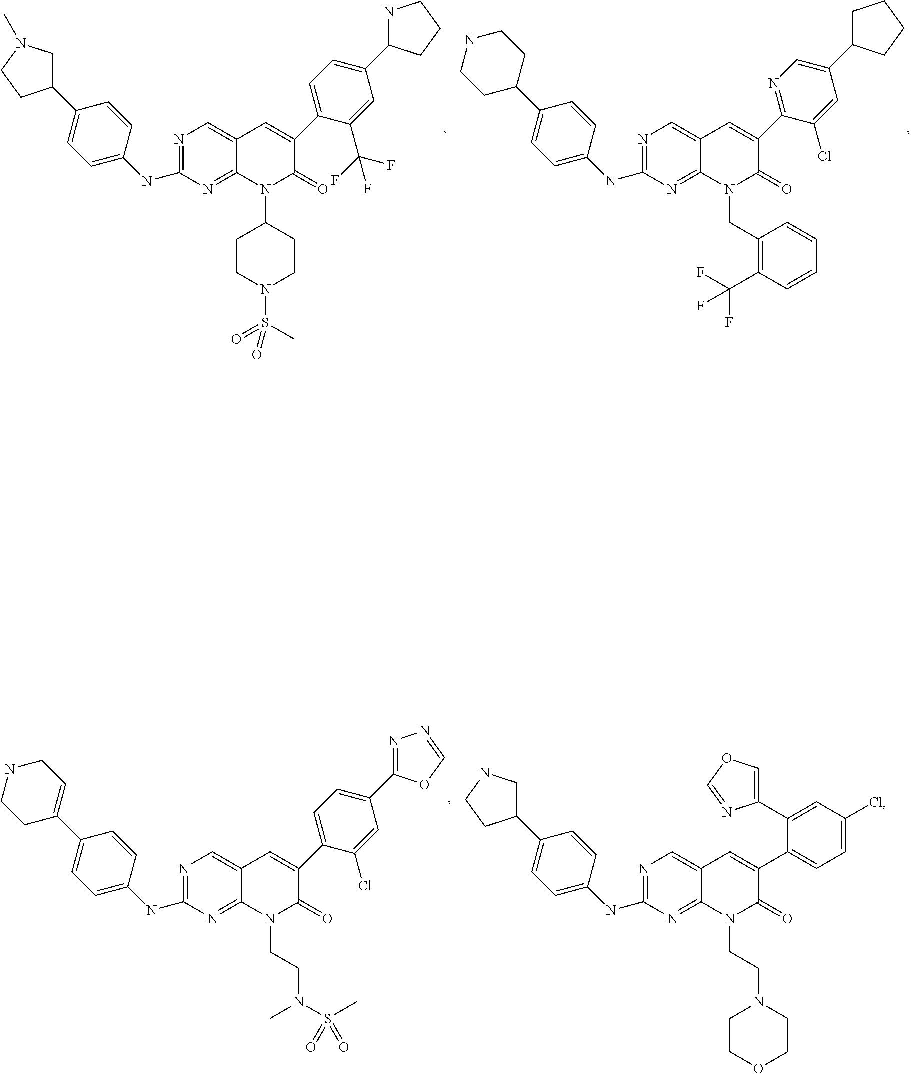

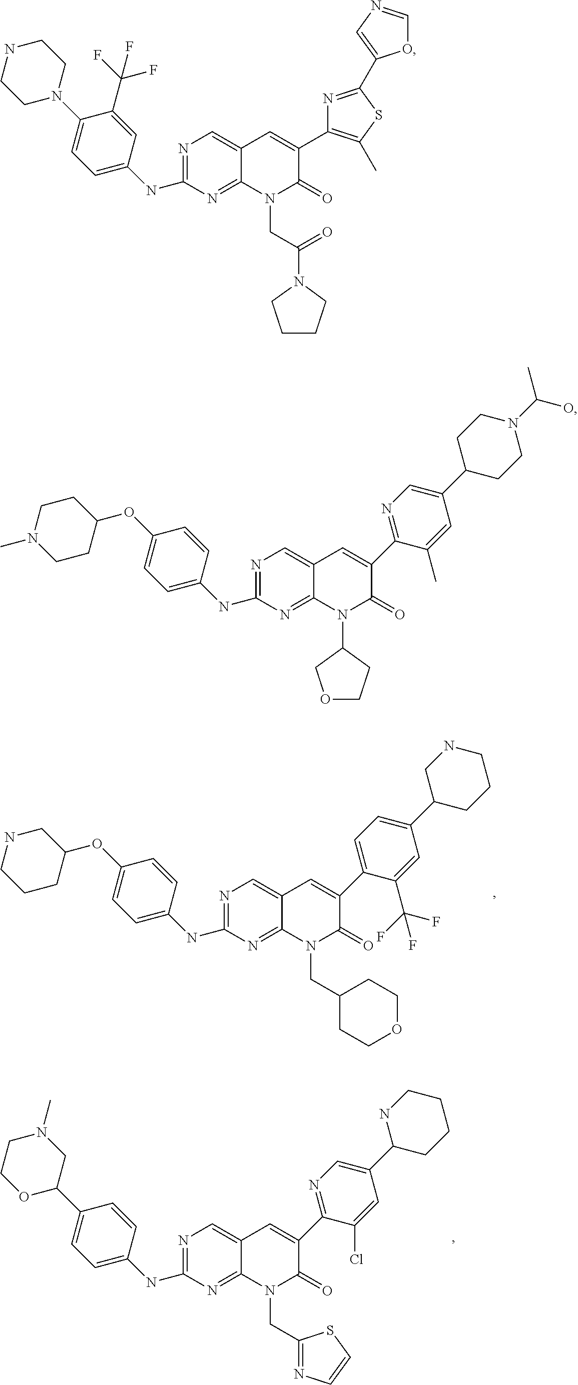

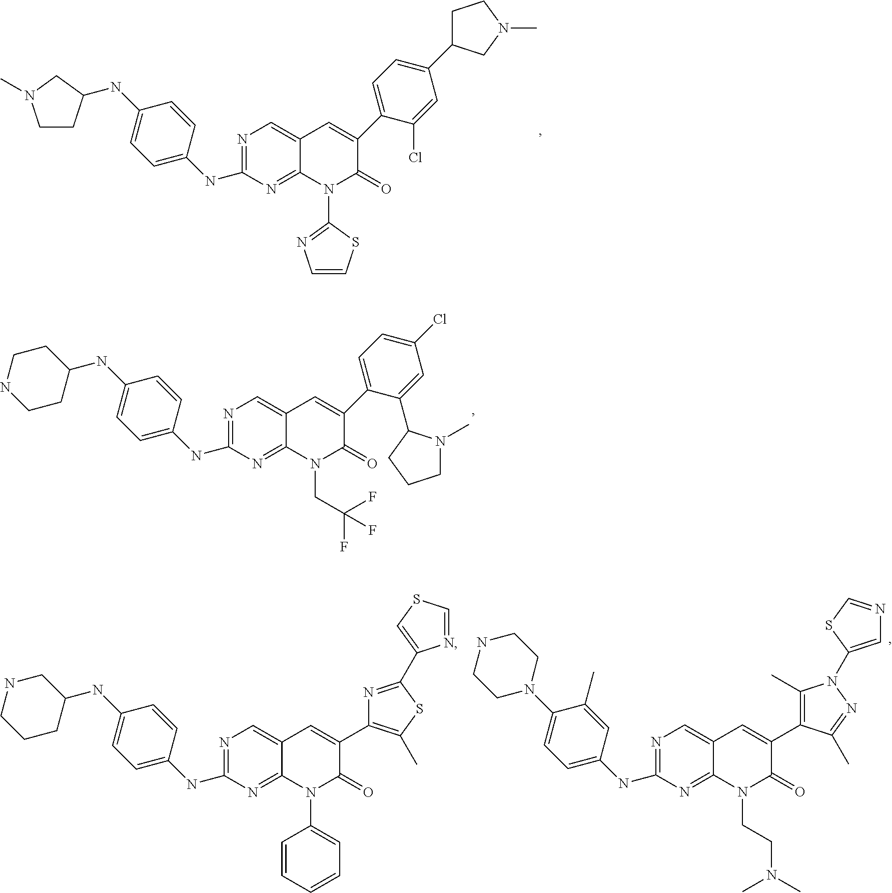

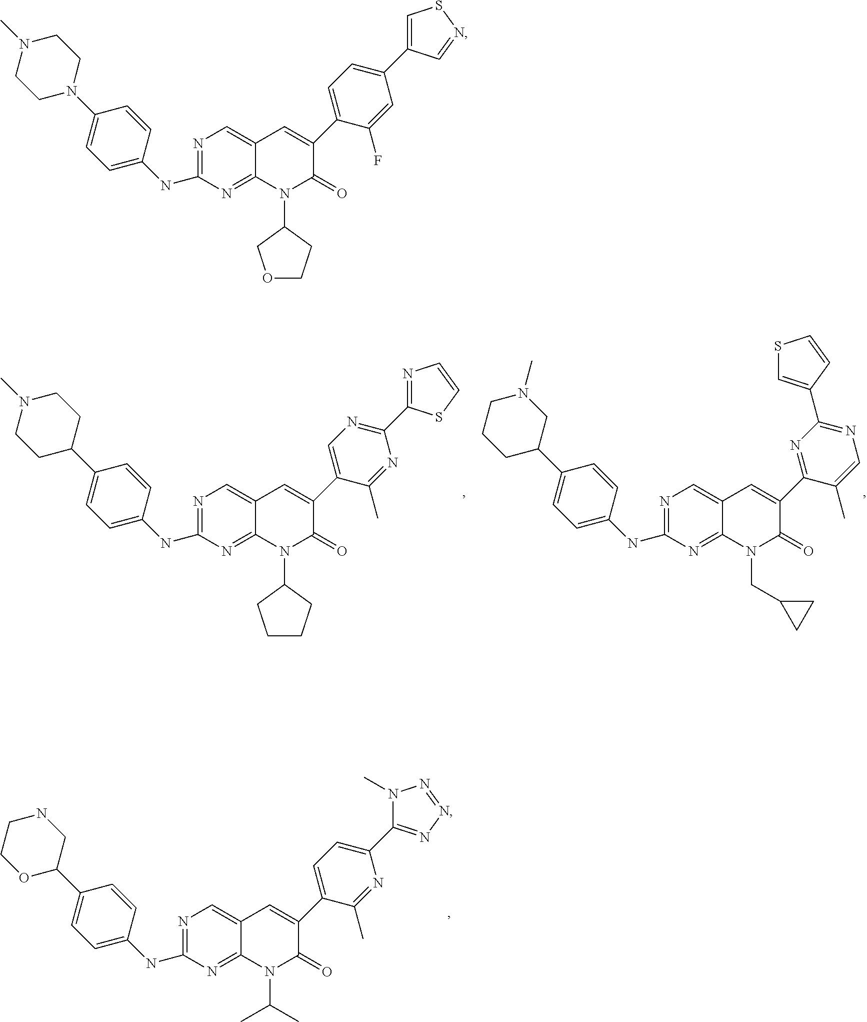

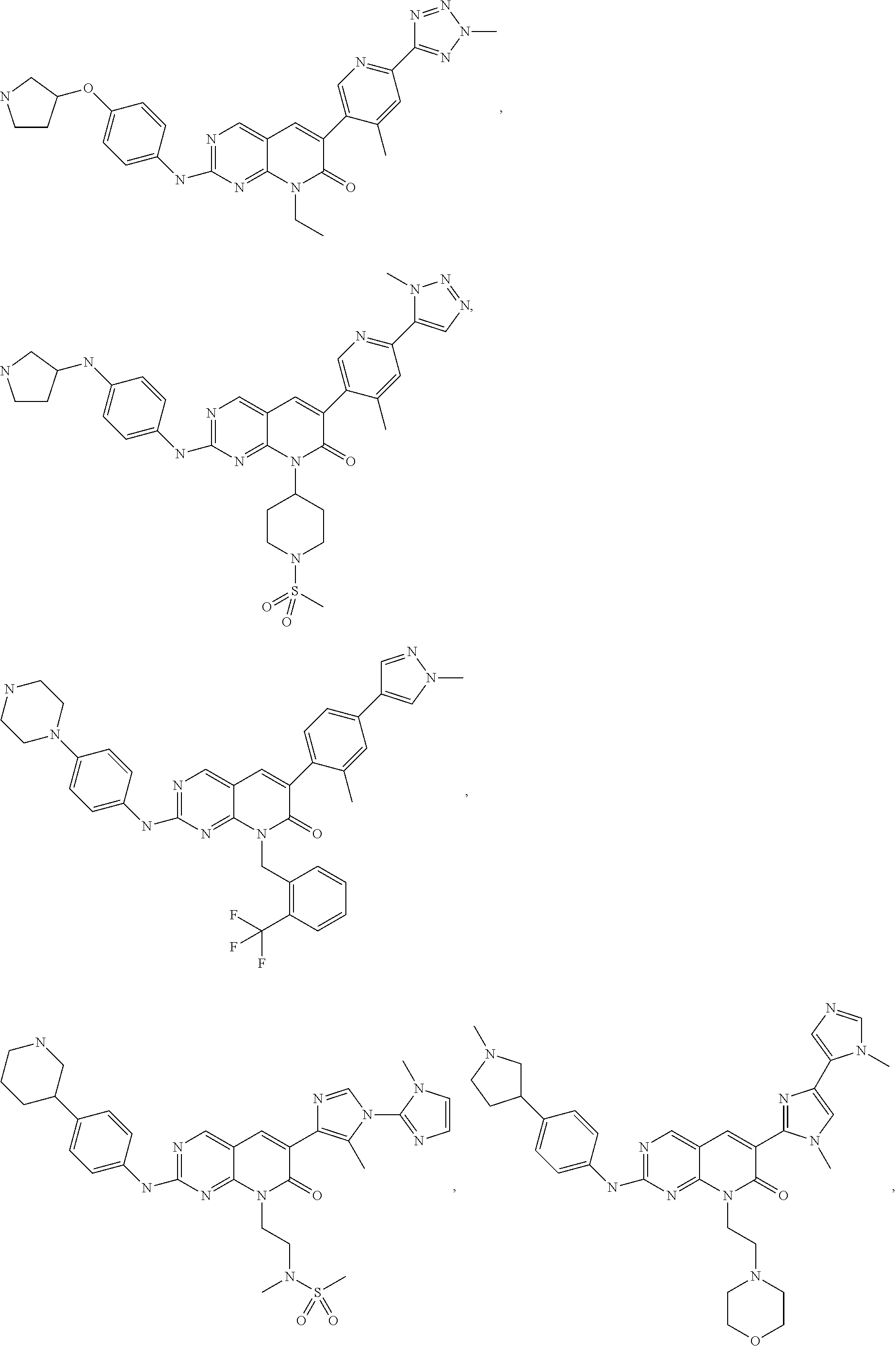

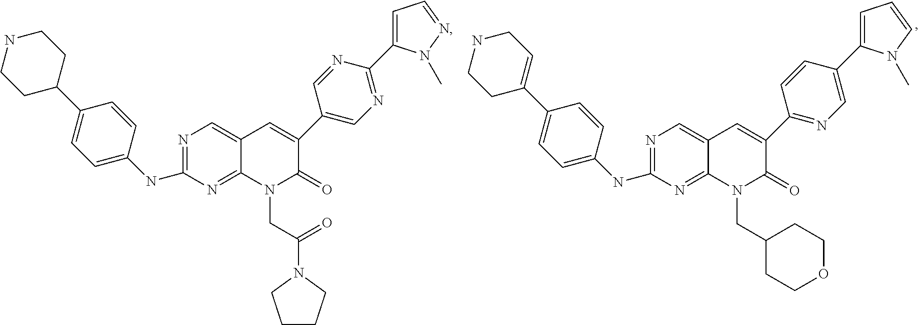

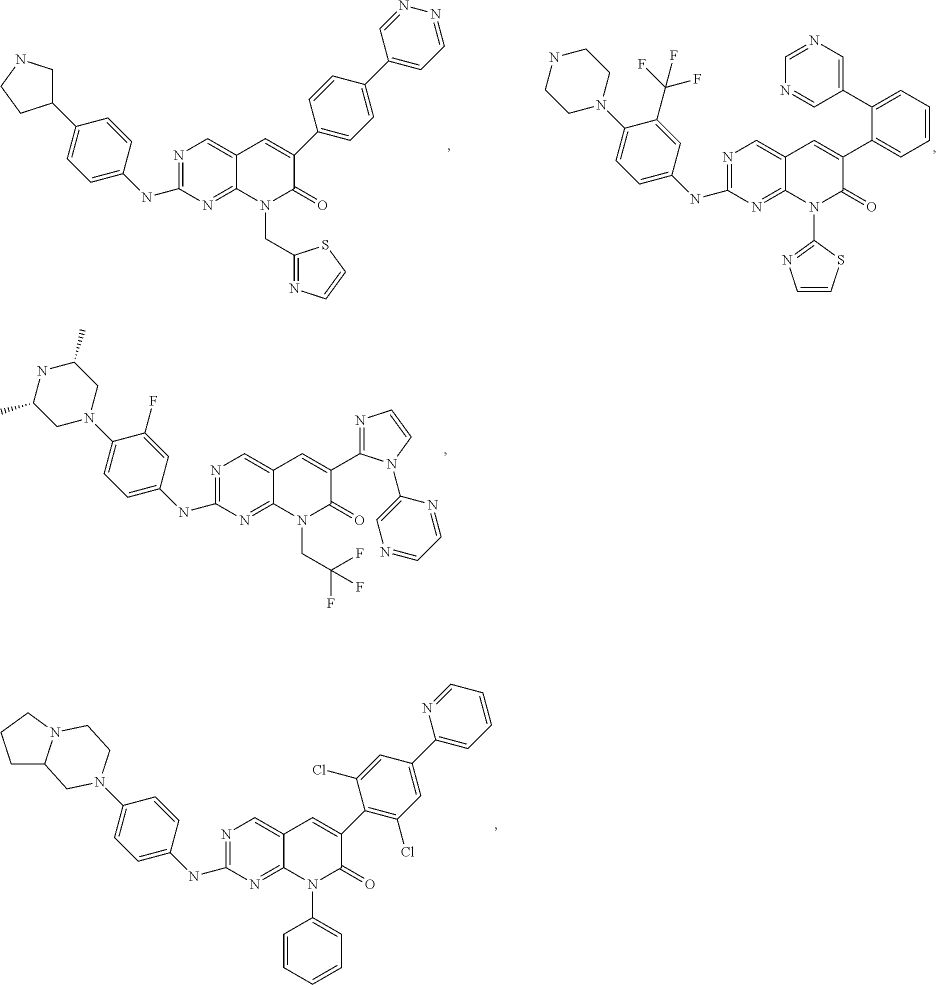 [SMILES: O=c1c(-c2ccc(-c3ccnnc3)cc2)cc2cnc(Nc3ccc(C4CCNC4)cc3)nc2n1Cc1nccs1 ; O=c1c(-c2ccccc2-c2cncnc2)cc2cnc(Nc3ccc(N4CCNCC4)c(C(F)(F)F)c3)nc2n1-c1nccs1 ; C[C@@H]1CN(c2ccc(Nc3ncc4cc(-c5nccn5-c5cnccn5)c(=O)n(CC(F)(F)F)c4n3)cc2F)C[C@H](C)N1 ; O=c1c(-c2c(Cl)cc(-c3ccccn3)cc2Cl)cc2cnc(Nc3ccc(N4CCN5CCCC5C4)cc3)nc2n1-c1ccccc1]

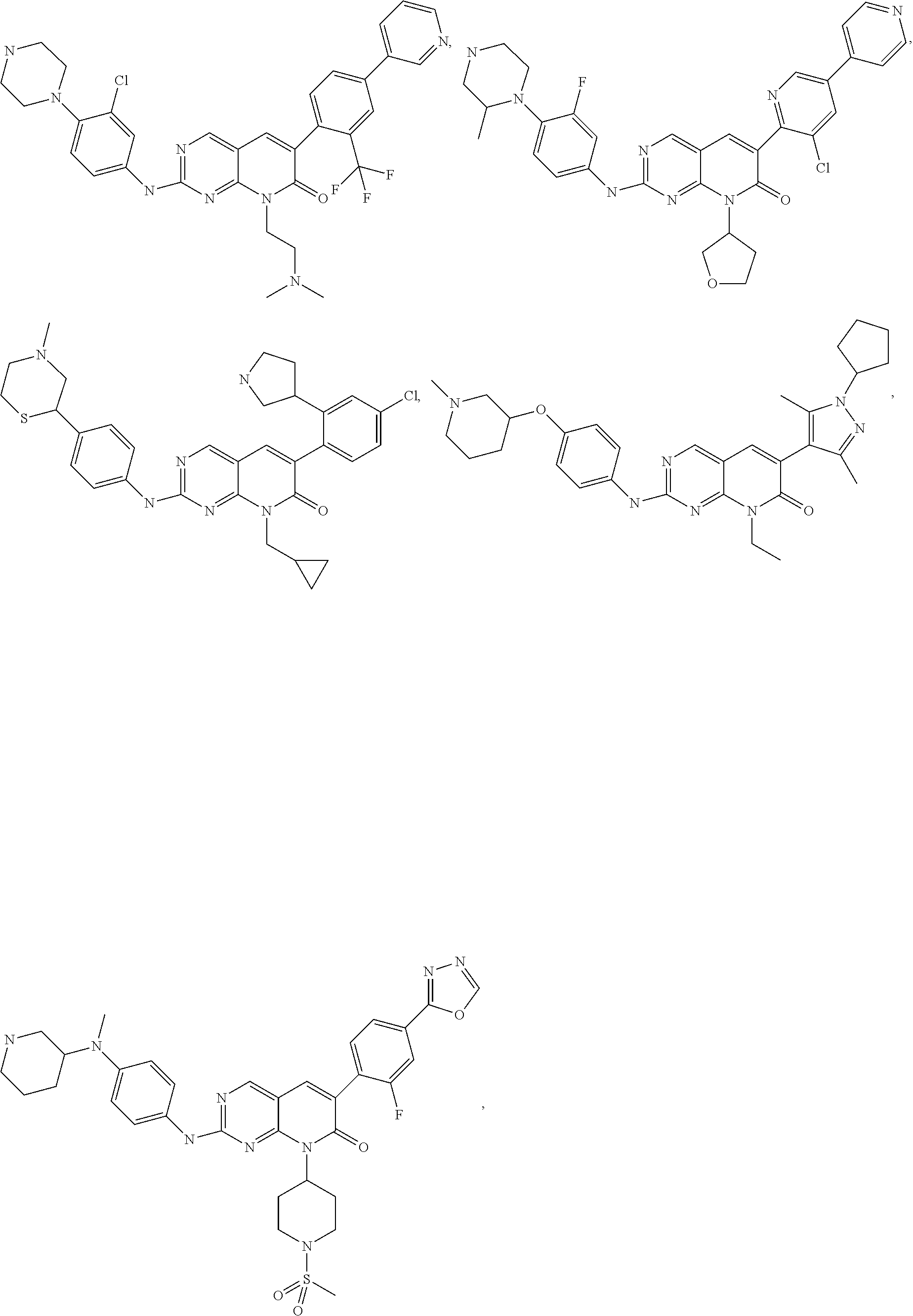

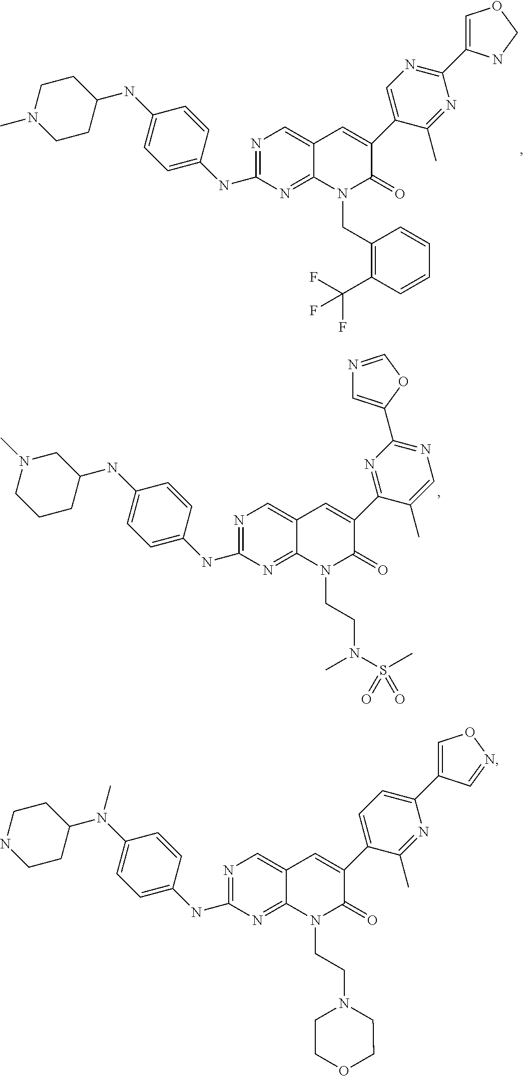

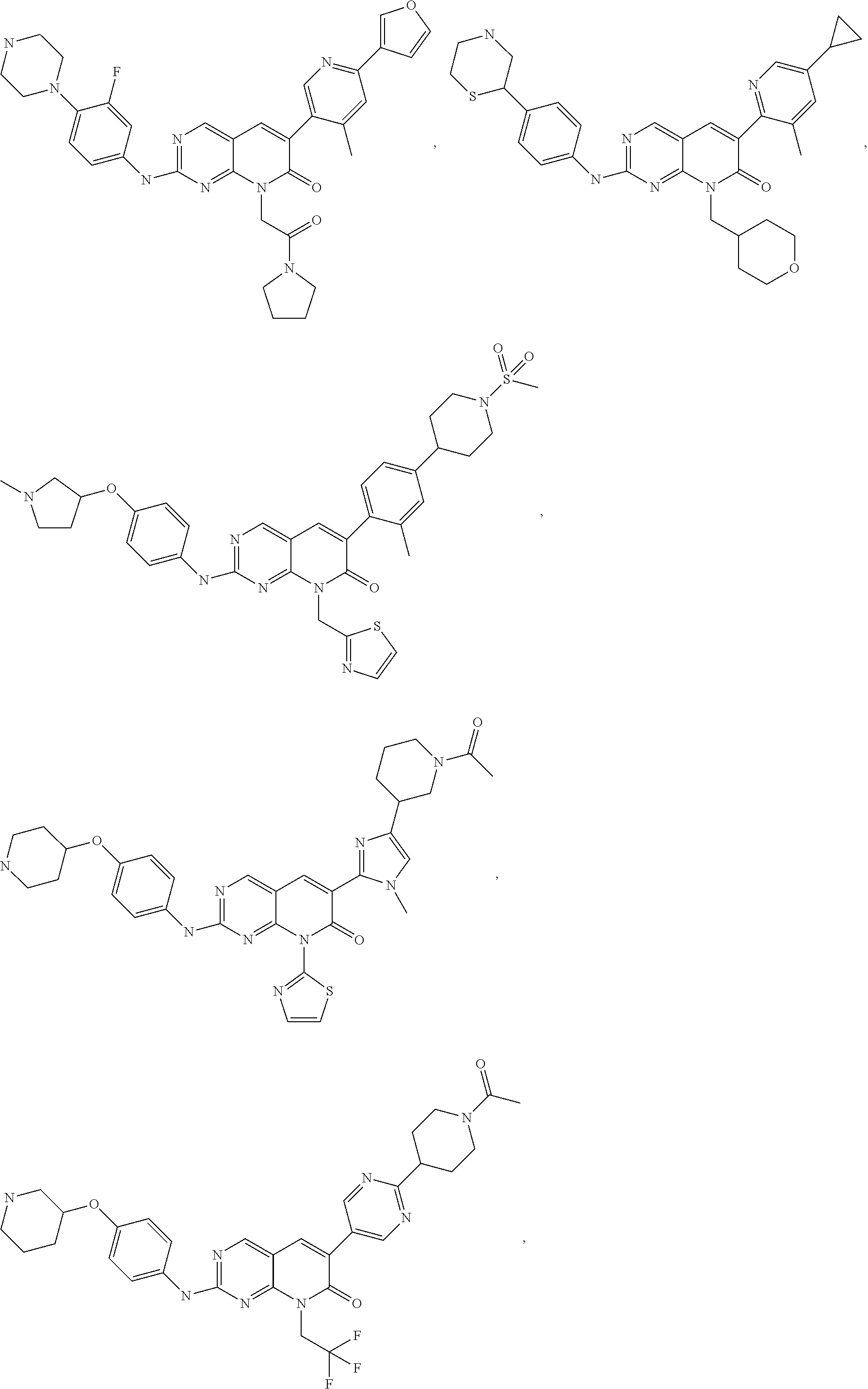

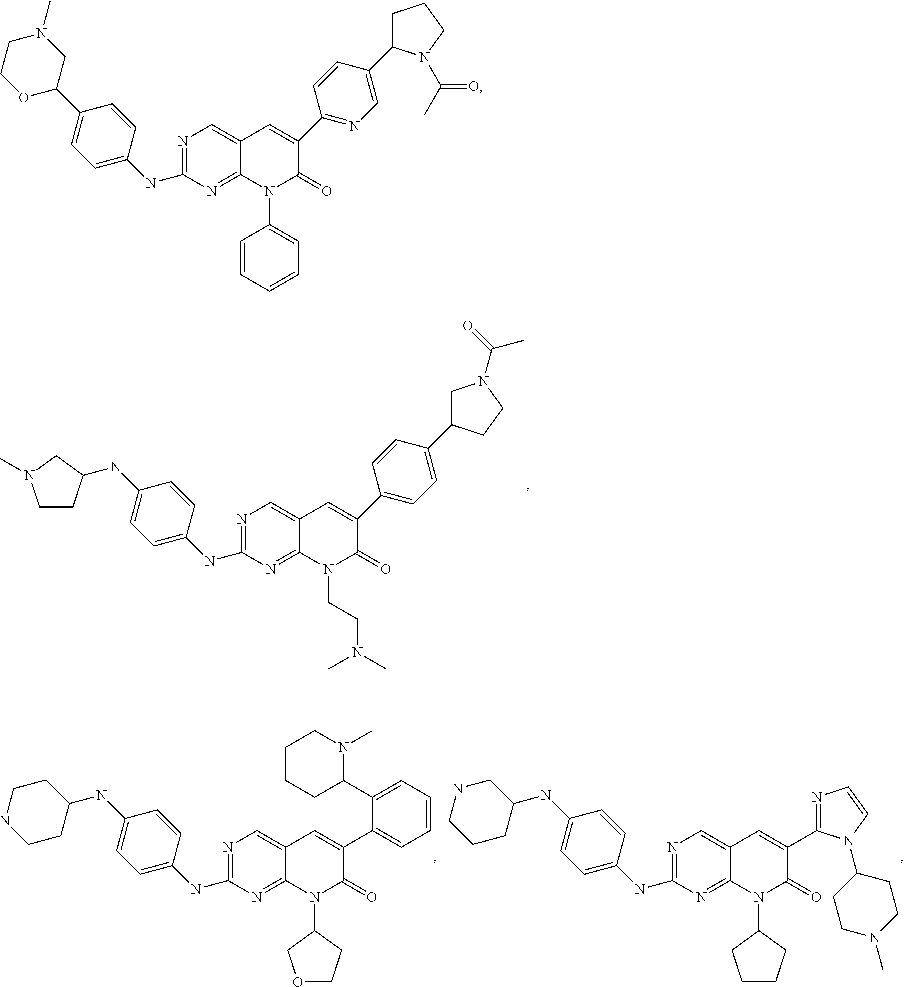

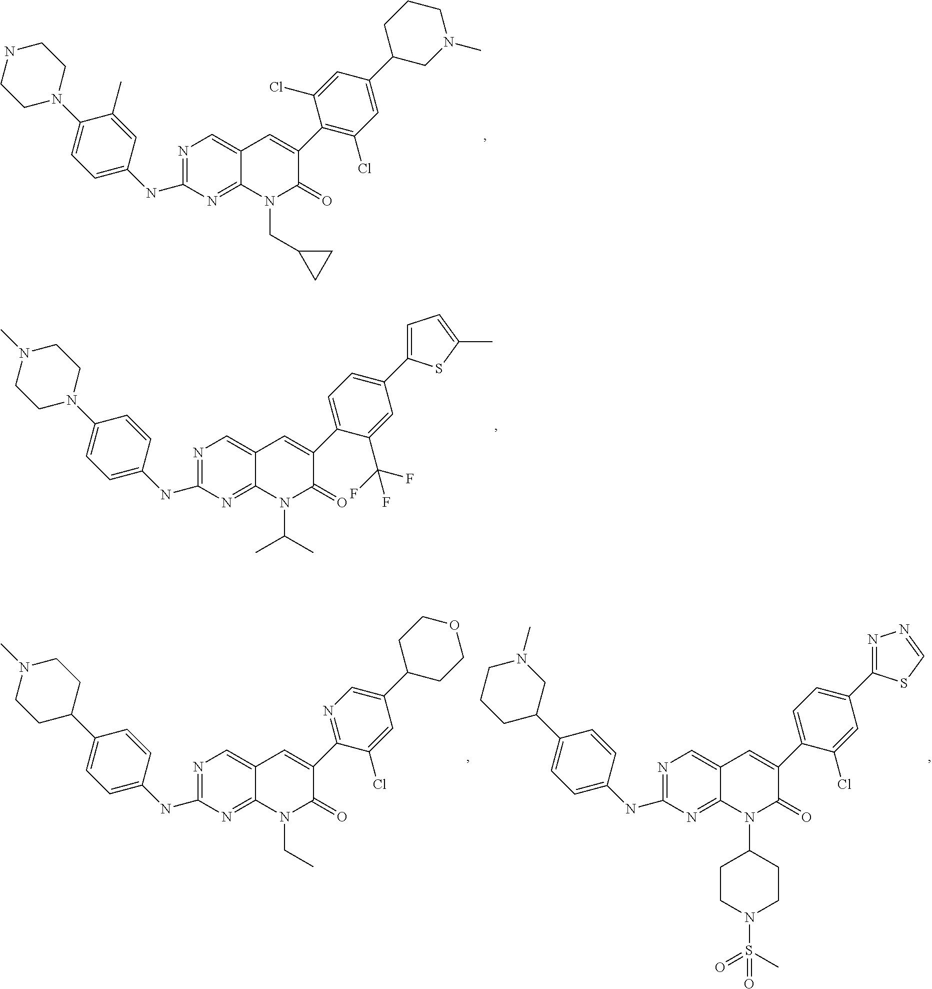

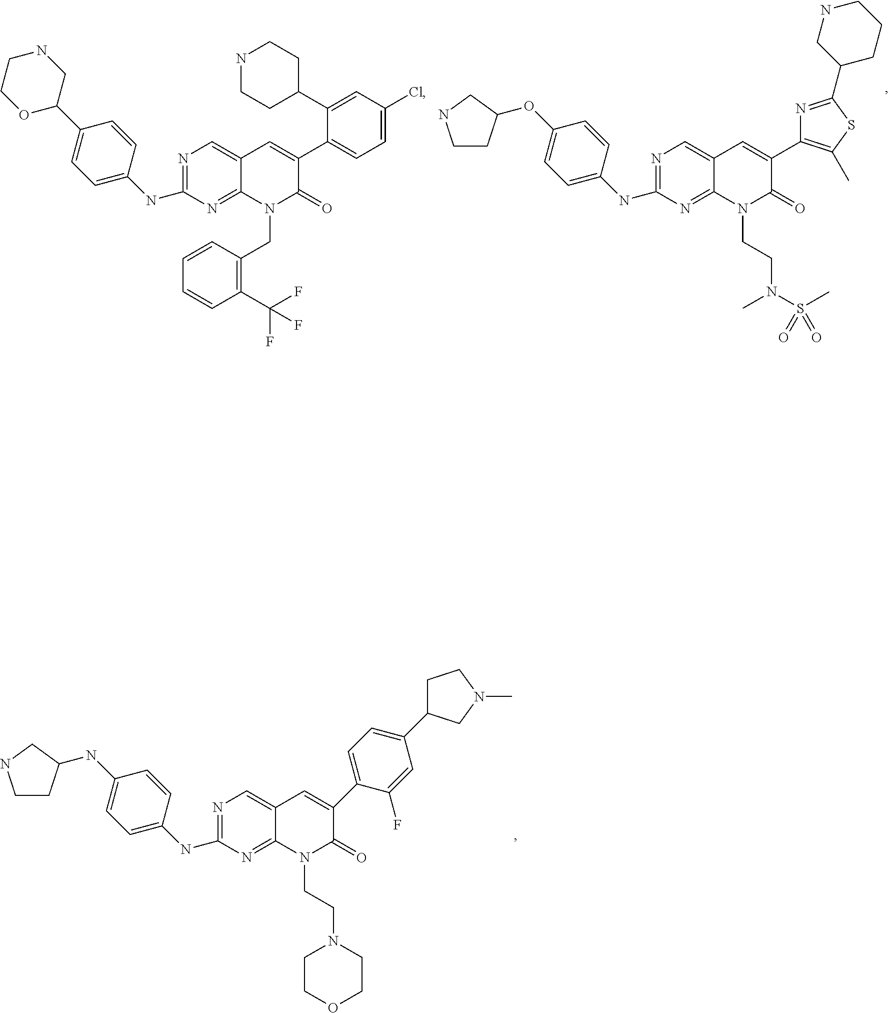

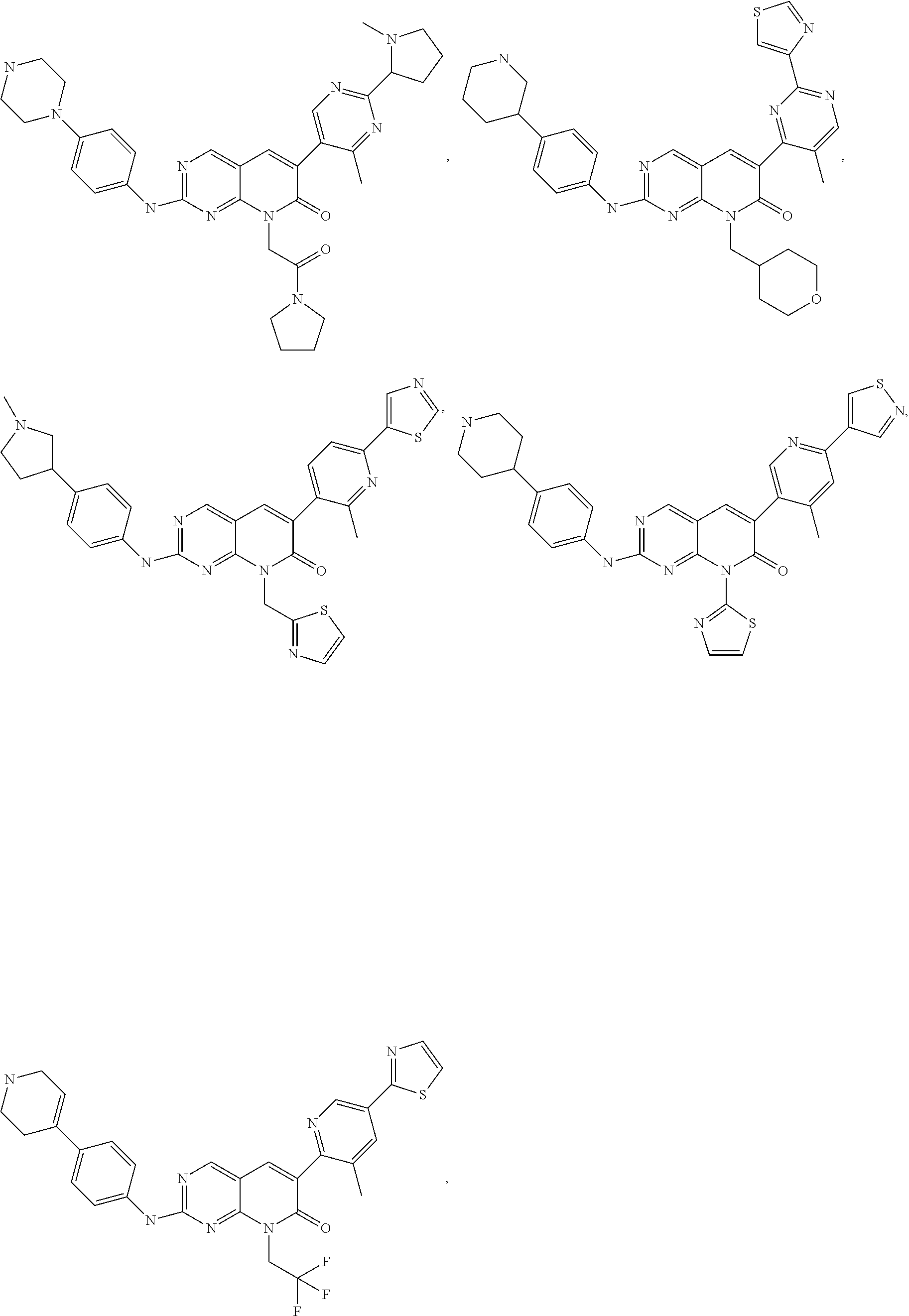

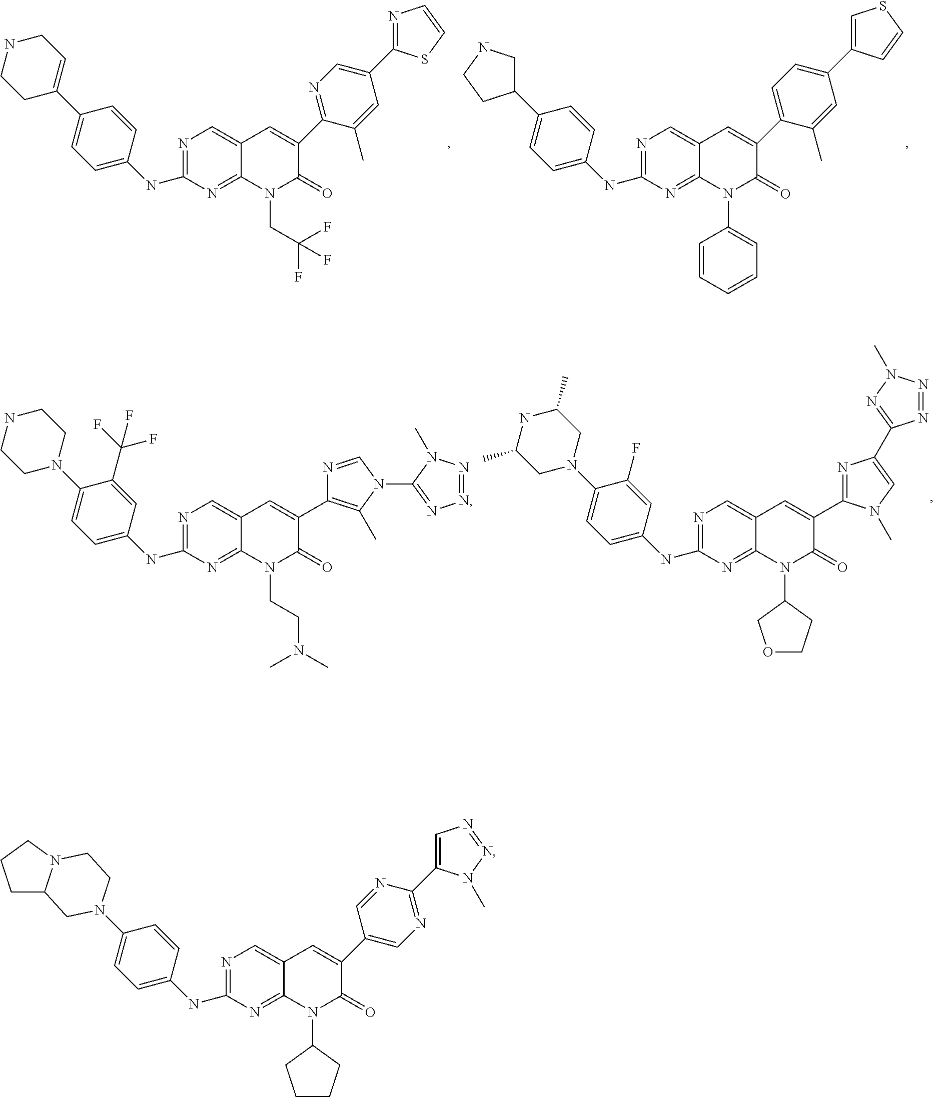

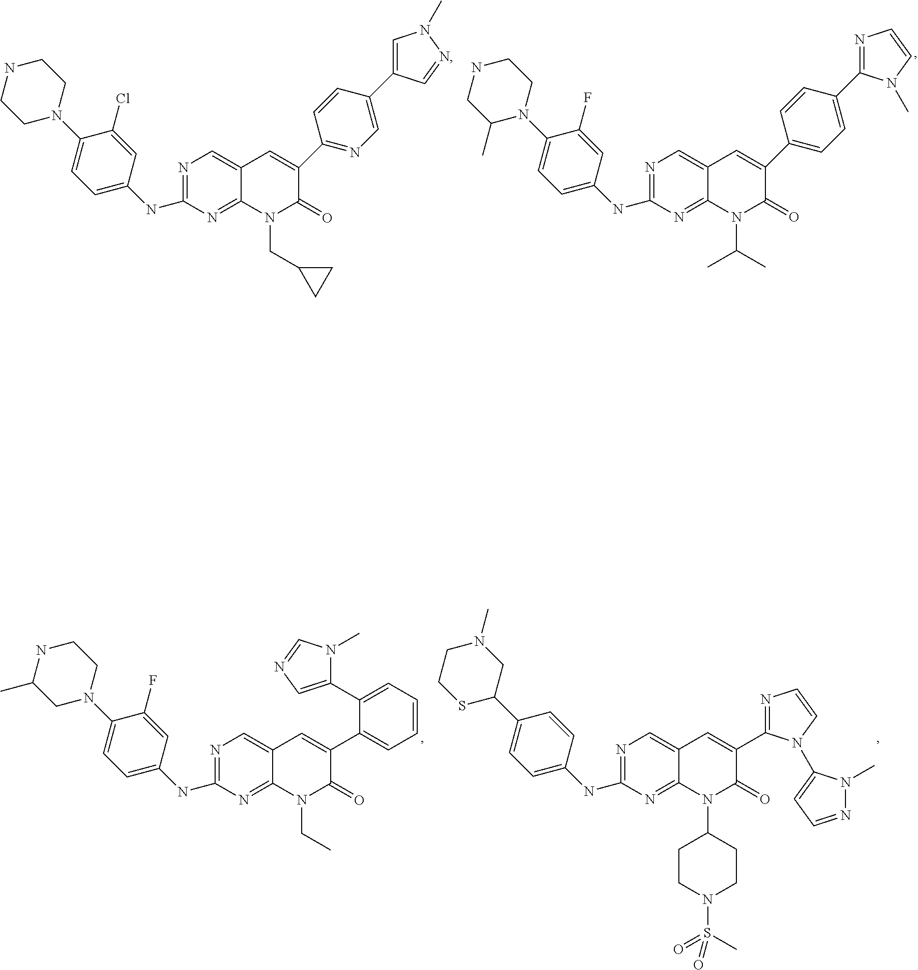

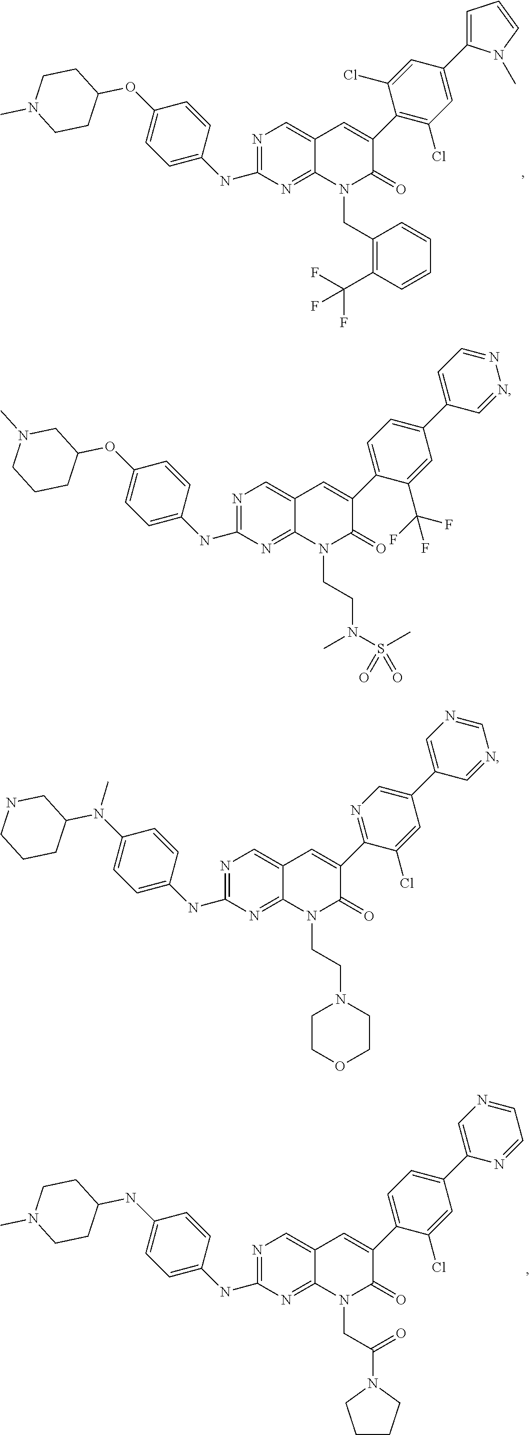 [SMILES: CN1CCC(Oc2ccc(Nc3ncc4cc(-c5c(Cl)cc(-c6cccn6C)cc5Cl)c(=O)n(Cc5ccccc5C(F)(F)F)c4n3)cc2)CC1 ; CN1CCCC(Oc2ccc(Nc3ncc4cc(-c5ccc(-c6ccnnc6)cc5C(F)(F)F)c(=O)n(CCN(C)S(C)(=O)=O)c4n3)cc2)C1 ; CN(c1ccc(Nc2ncc3cc(-c4ncc(-c5cncnc5)cc4Cl)c(=O)n(CCN4CCOCC4)c3n2)cc1)C1CCCNC1 ; CN1CCC(Nc2ccc(Nc3ncc4cc(-c5ccc(-c6cnccn6)cc5Cl)c(=O)n(CC(=O)N5CCCC5)c4n3)cc2)CC1]

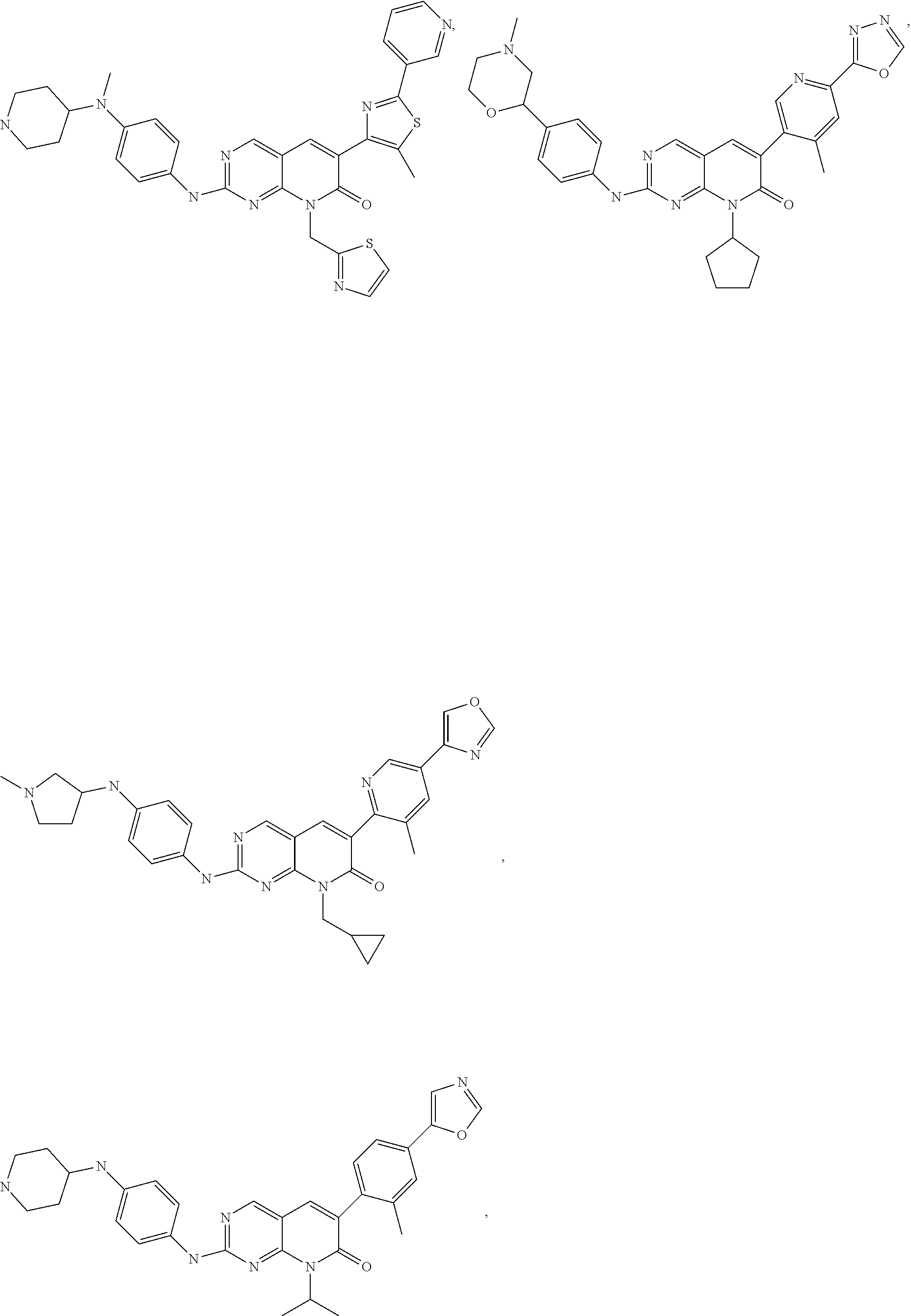

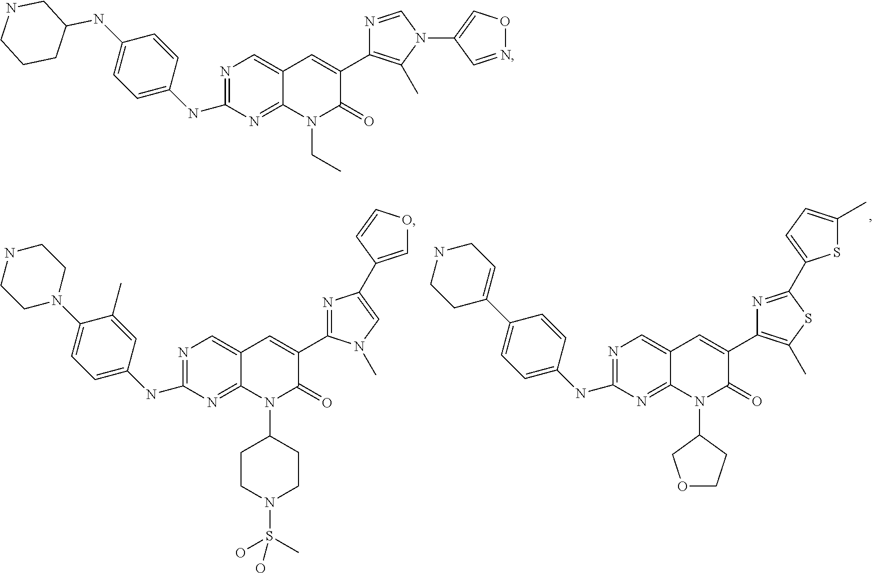

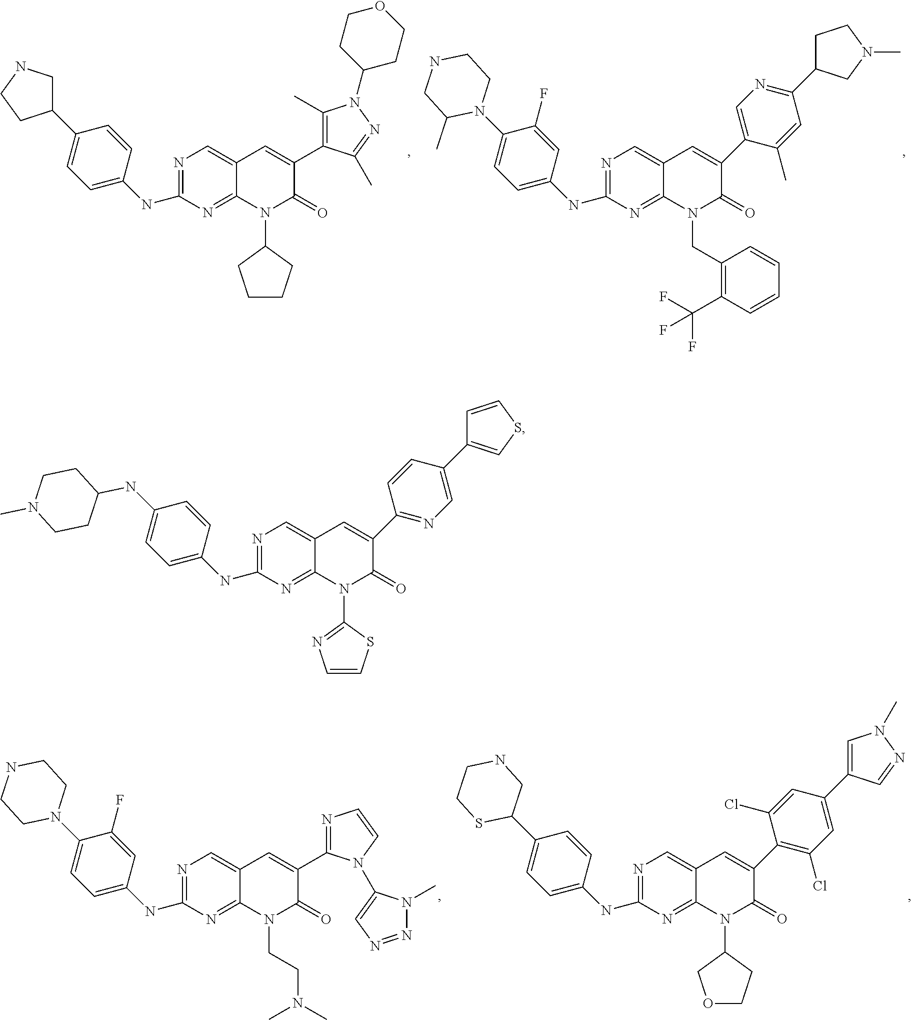

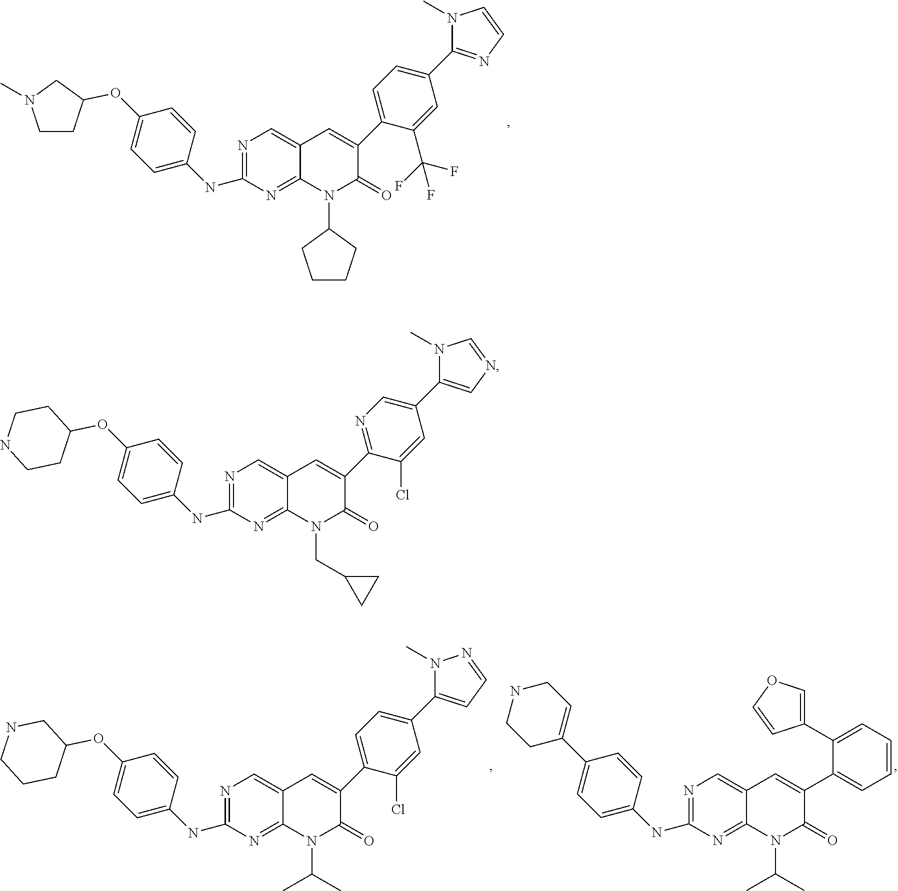

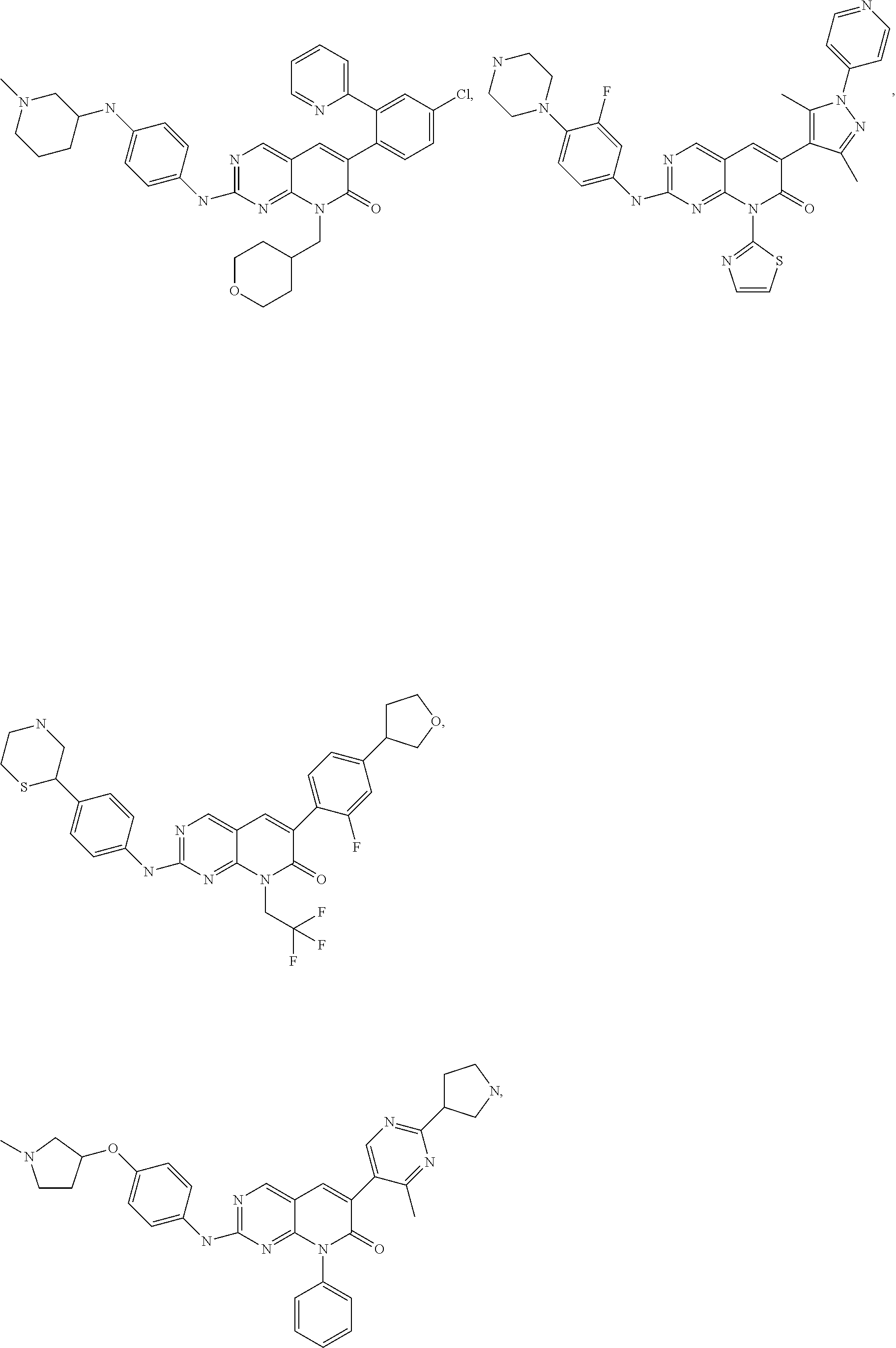

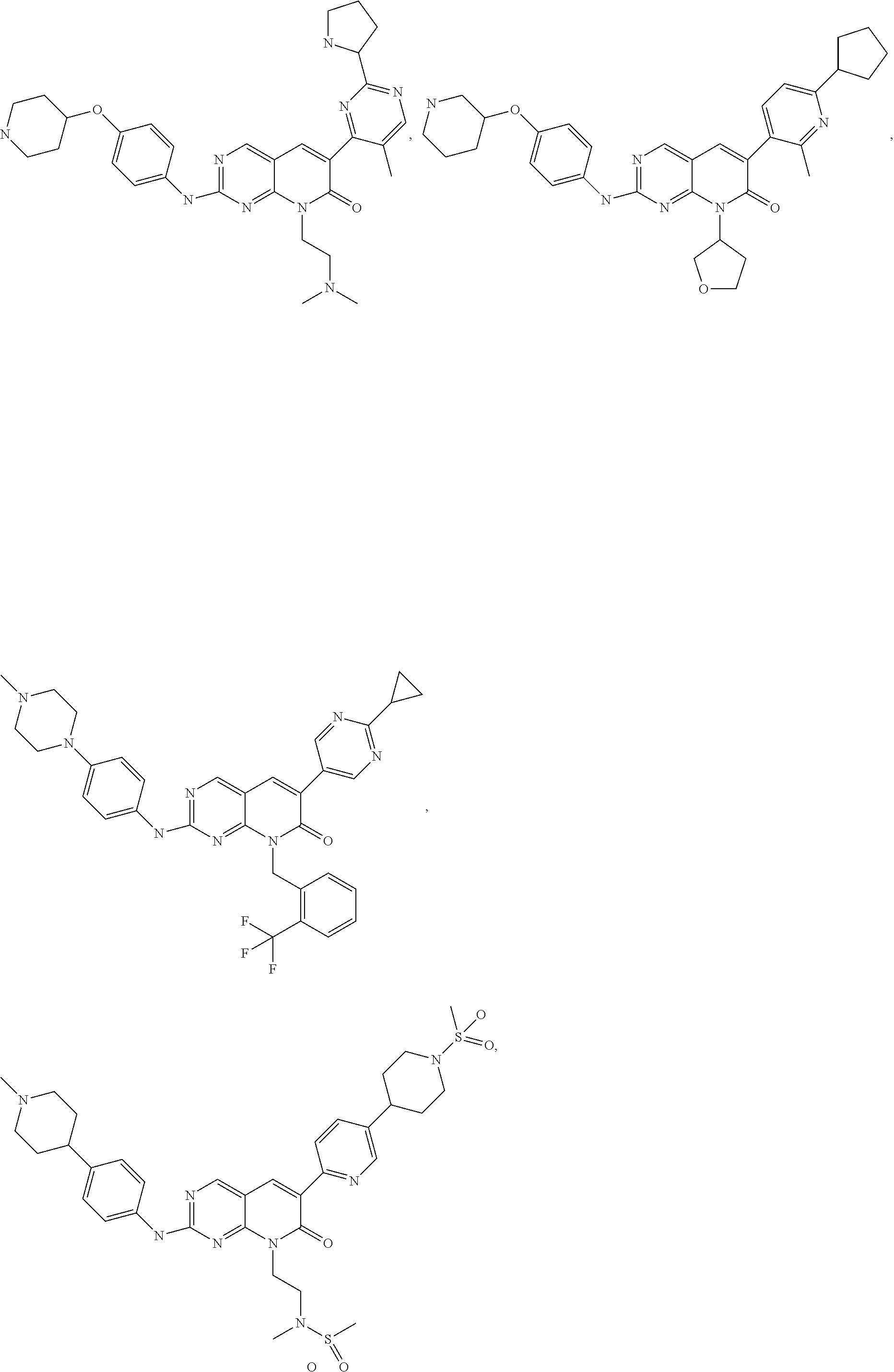

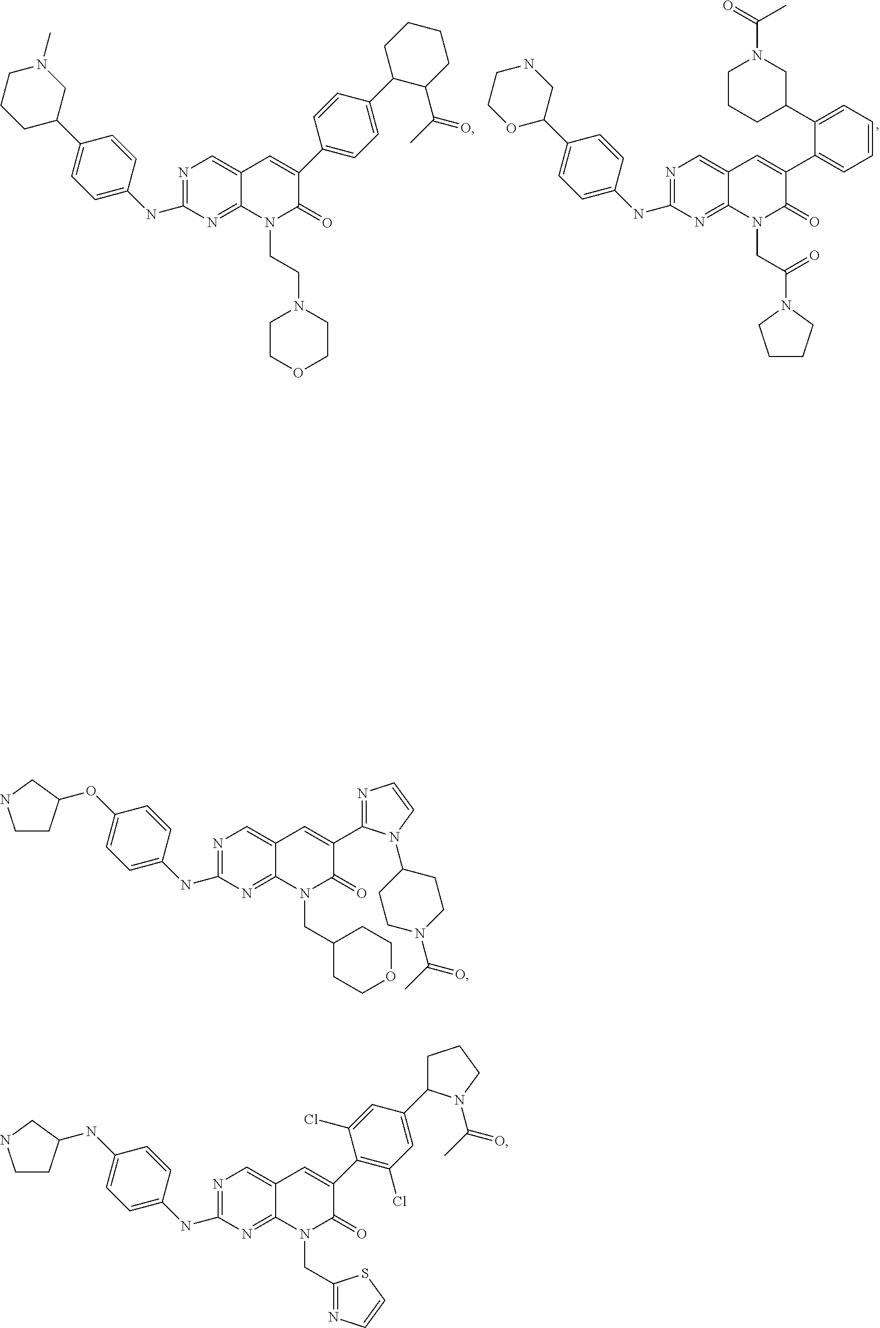

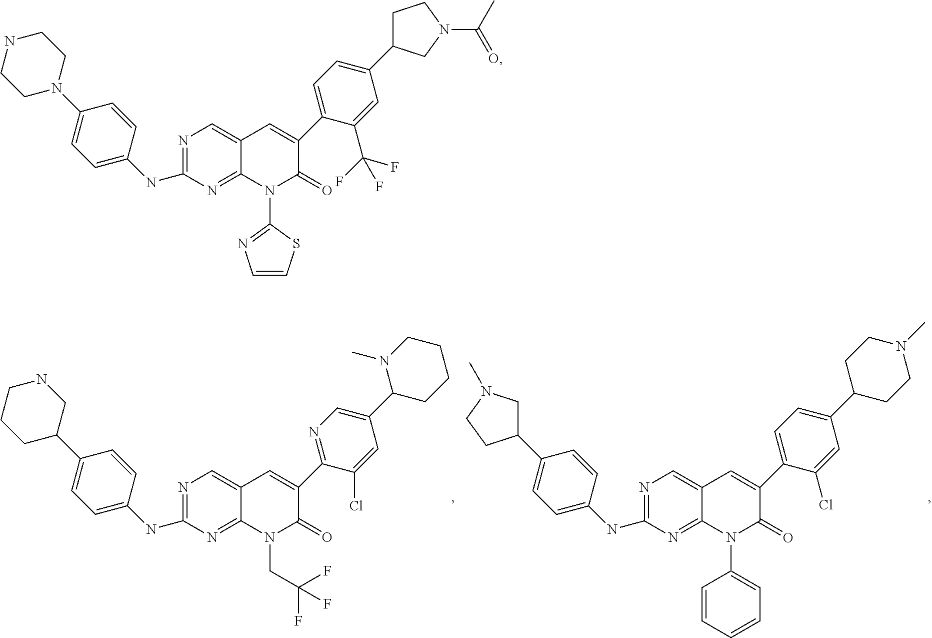

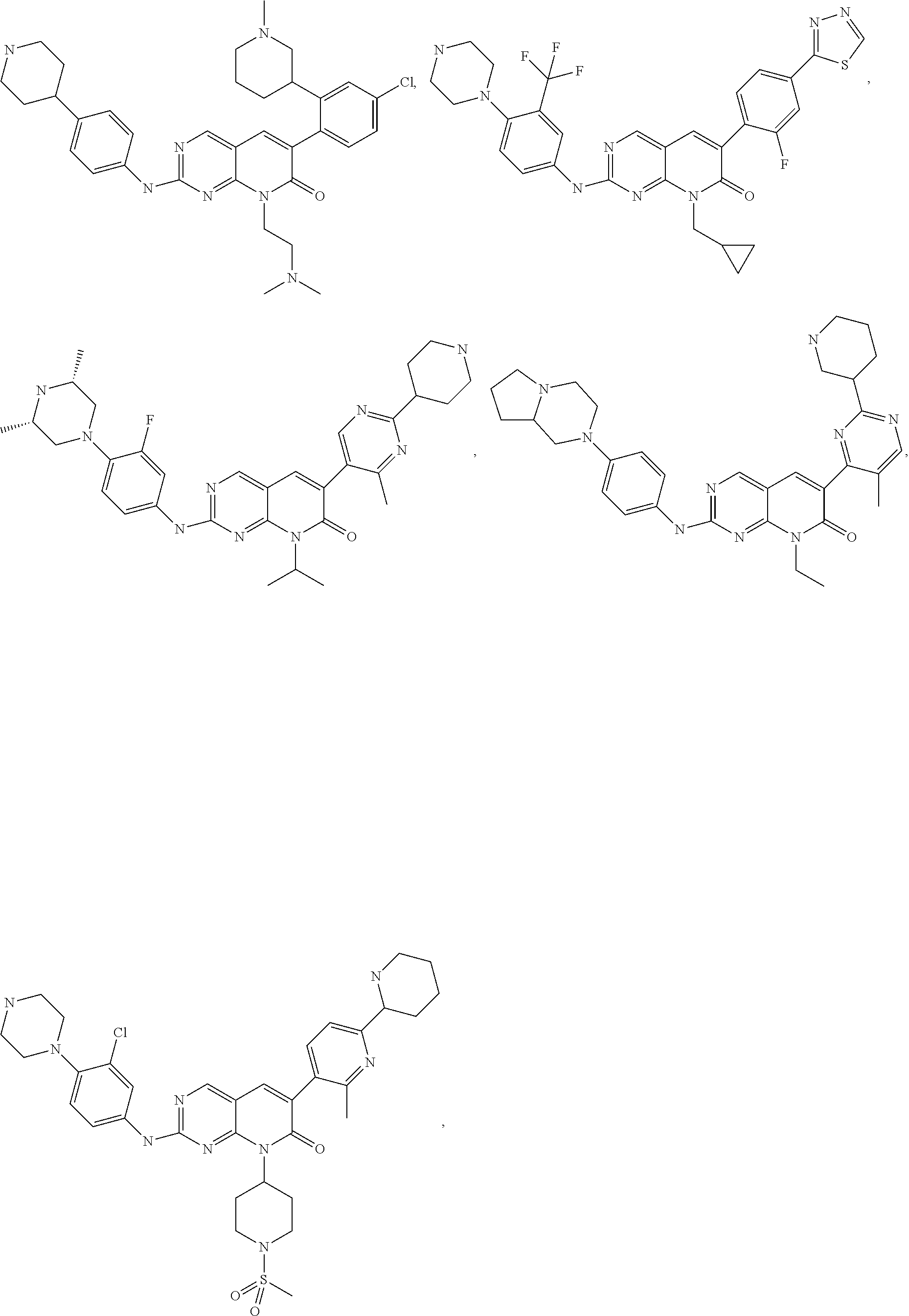

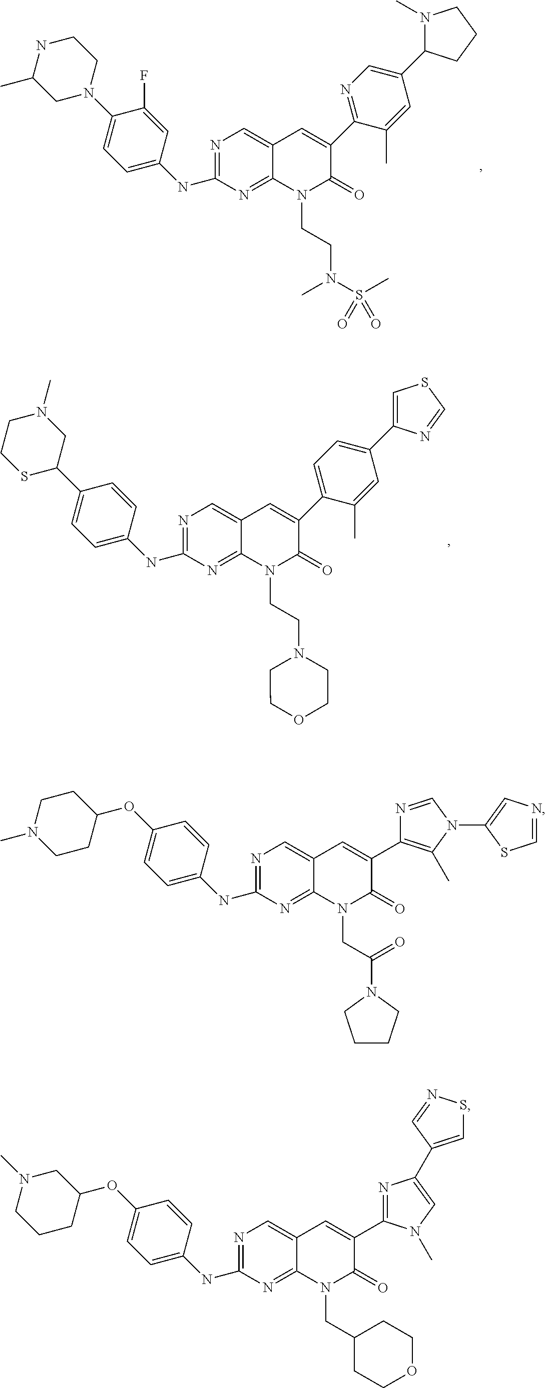 [SMILES: Cc1cc(C2CCCN2C)cnc1-c1cc2cnc(Nc3ccc(N4CCNC(C)C4)c(F)c3)nc2n(CCN(C)S(C)(=O)=O)c1=O ; Cc1cc(-c2cscn2)ccc1-c1cc2cnc(Nc3ccc(C4CN(C)CCS4)cc3)nc2n(CCN2CCOCC2)c1=O ; Cc1c(-c2cc3cnc(Nc4ccc(OC5CCN(C)CC5)cc4)nc3n(CC(=O)N3CCCC3)c2=O)ncn1-c1cncs1 ; CN1CCCC(Oc2ccc(Nc3ncc4cc(-c5nc(-c6cnsc6)cn5C)c(=O)n(CC5CCOCC5)c4n3)cc2)C1]

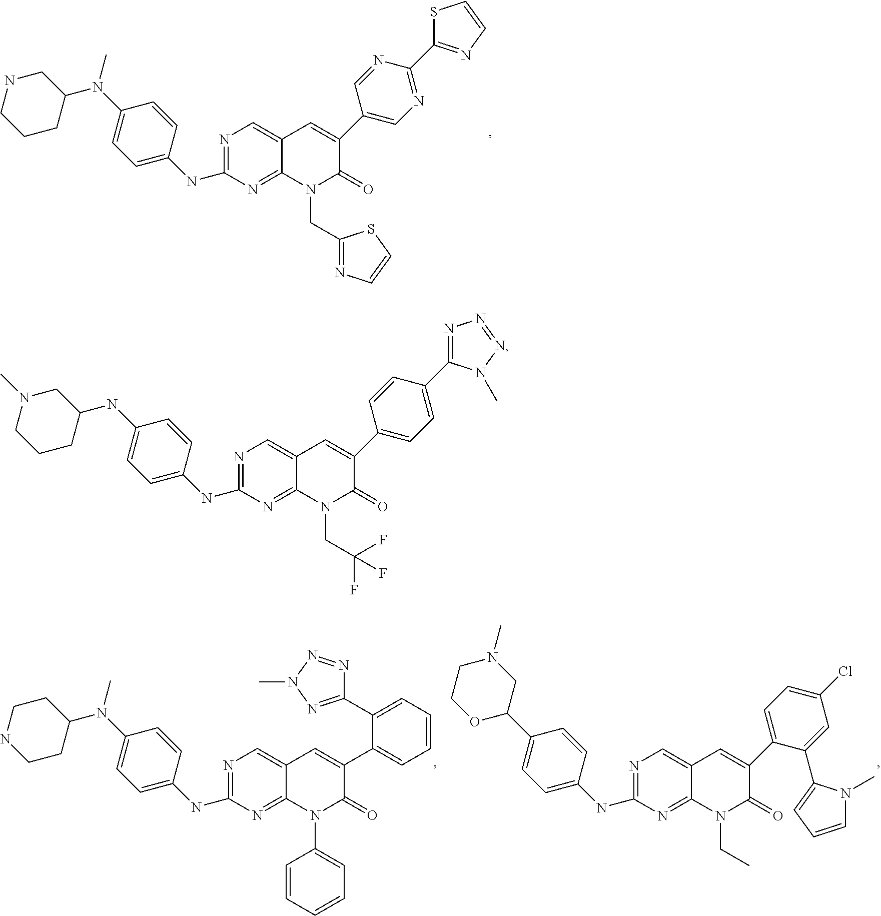

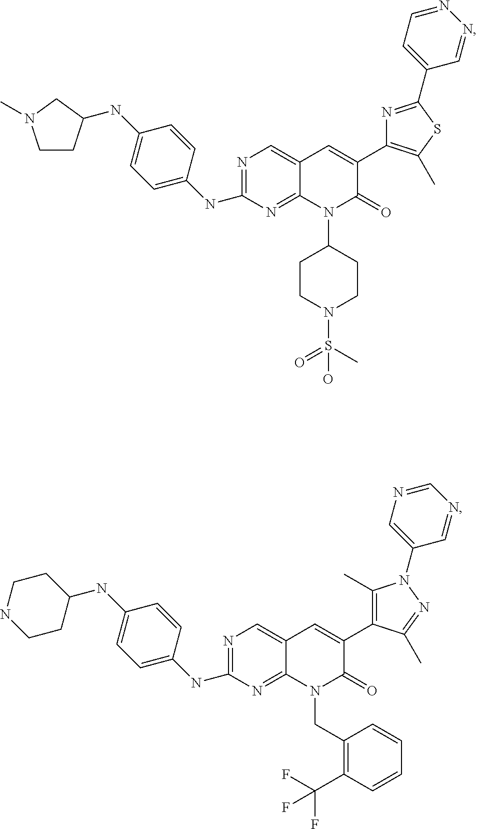

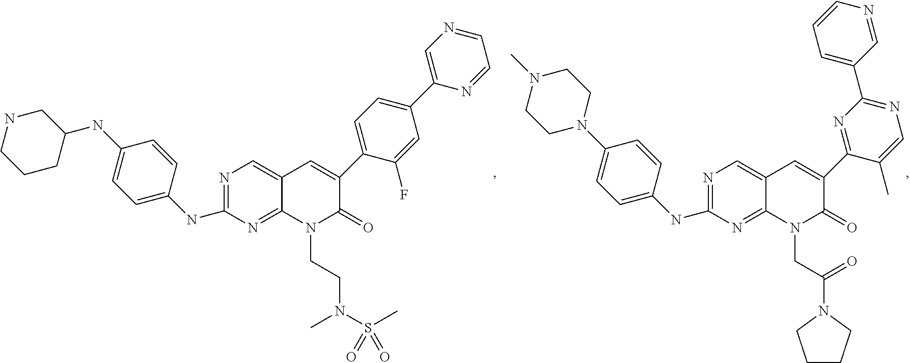

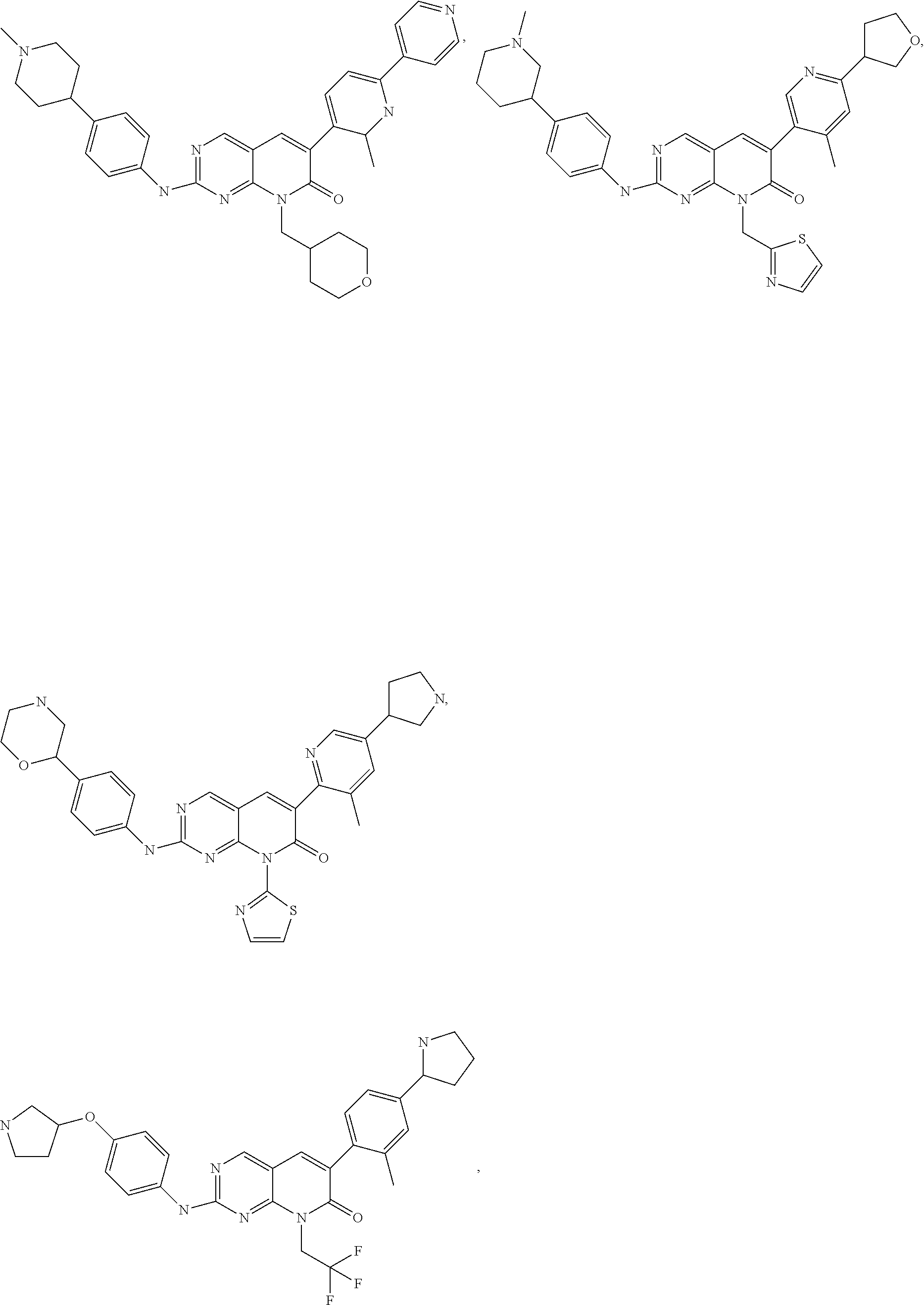

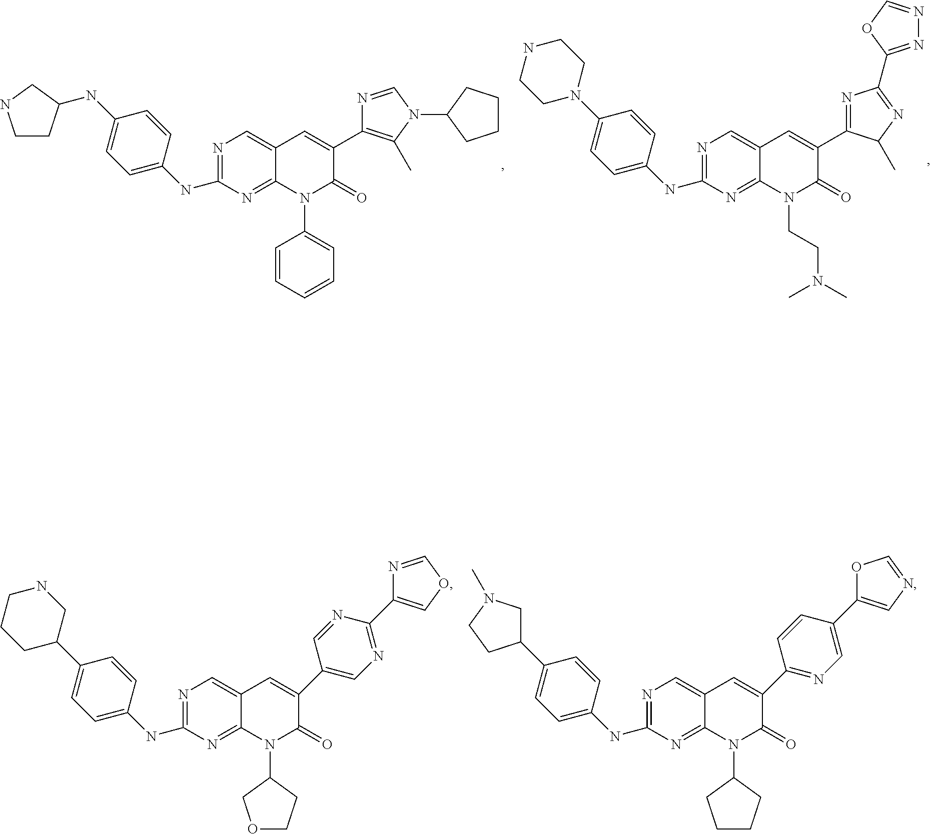

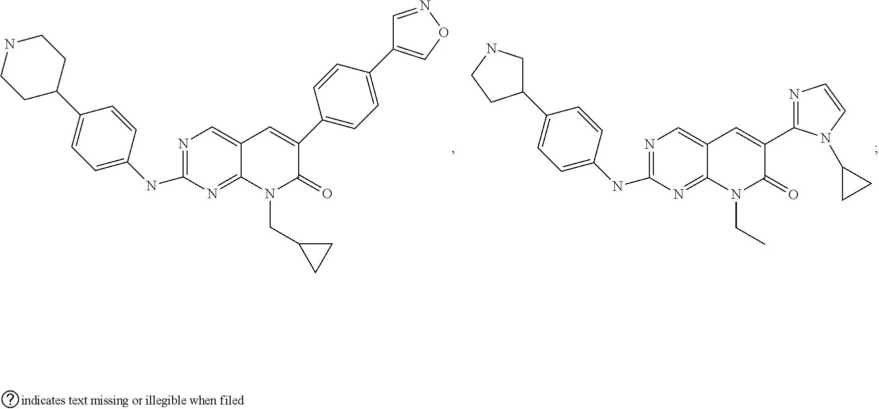 [SMILES: O=c1c(-c2ccc(-c3cnoc3)cc2)cc2cnc(Nc3ccc(C4CCNCC4)cc3)nc2n1CC1CC1 ; CCn1c(=O)c(-c2nccn2C2CC2)cc2cnc(Nc3ccc(C4CCNC4)cc3)nc21]

or a pharmaceutically acceptable salt, solvate or N-oxide thereof.

In a further embodiment is a compound having the structure:

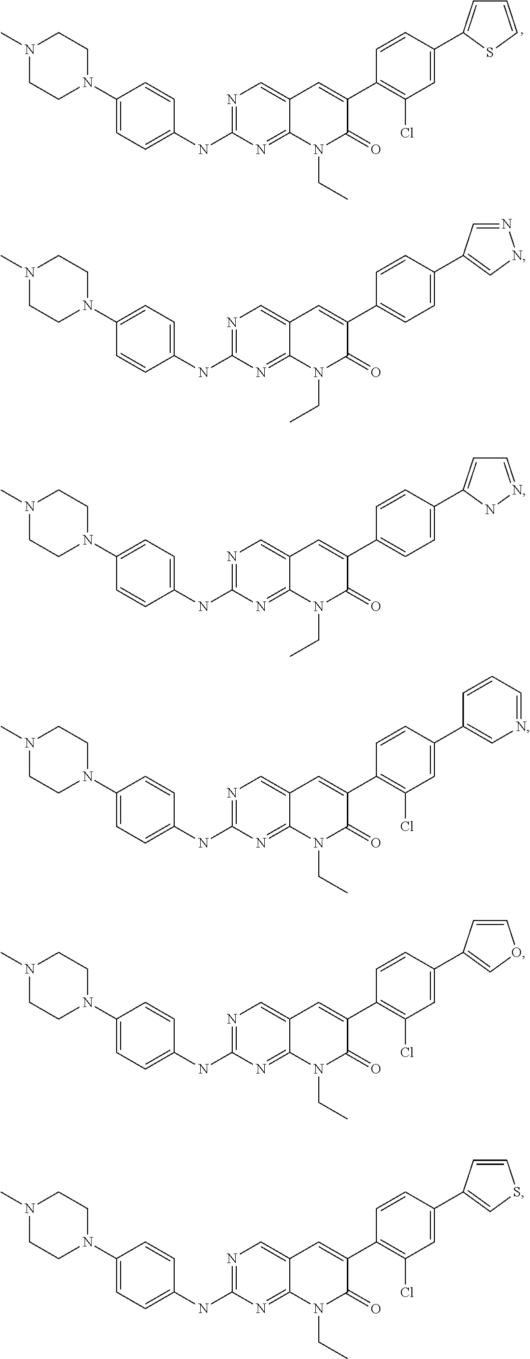

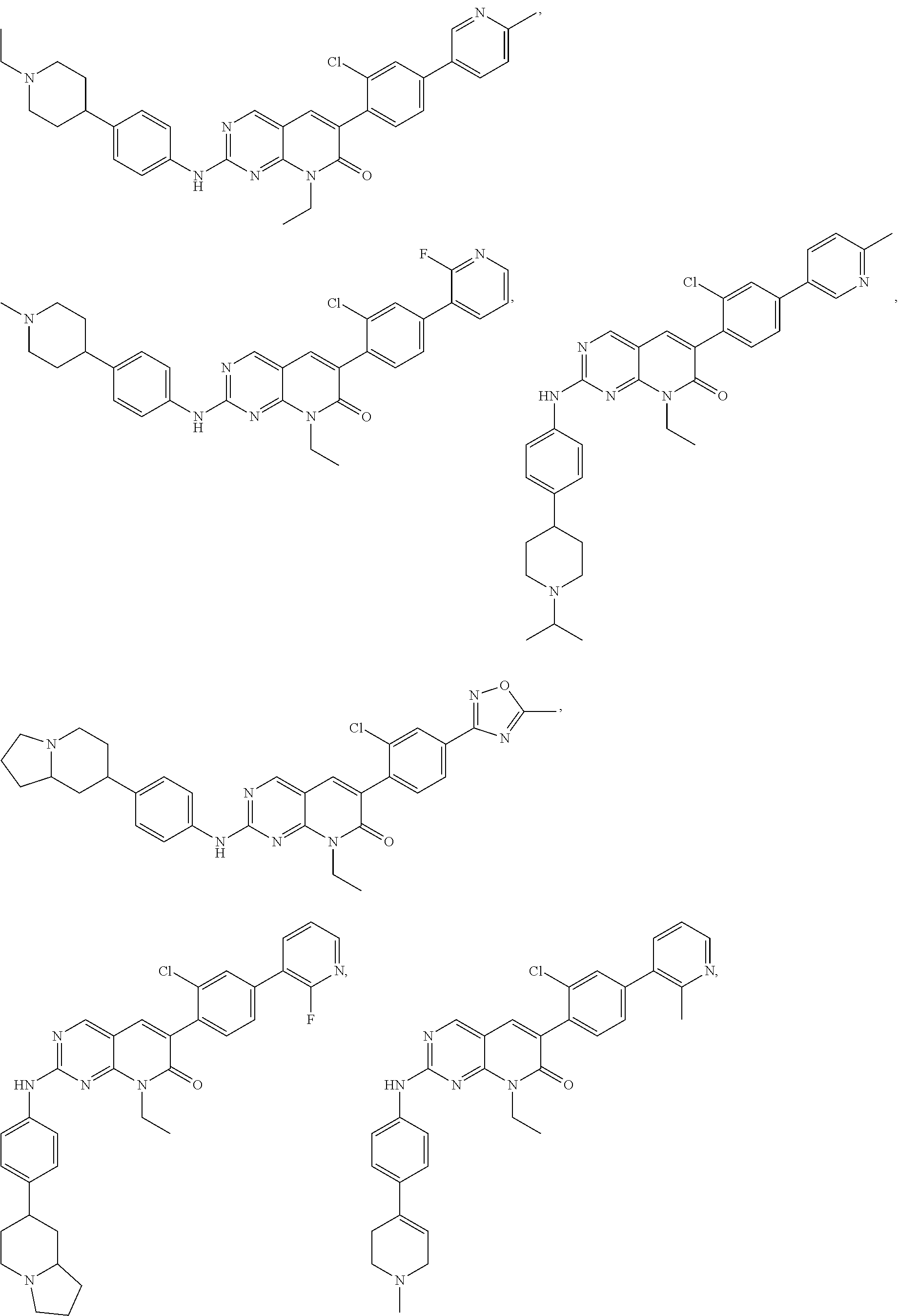

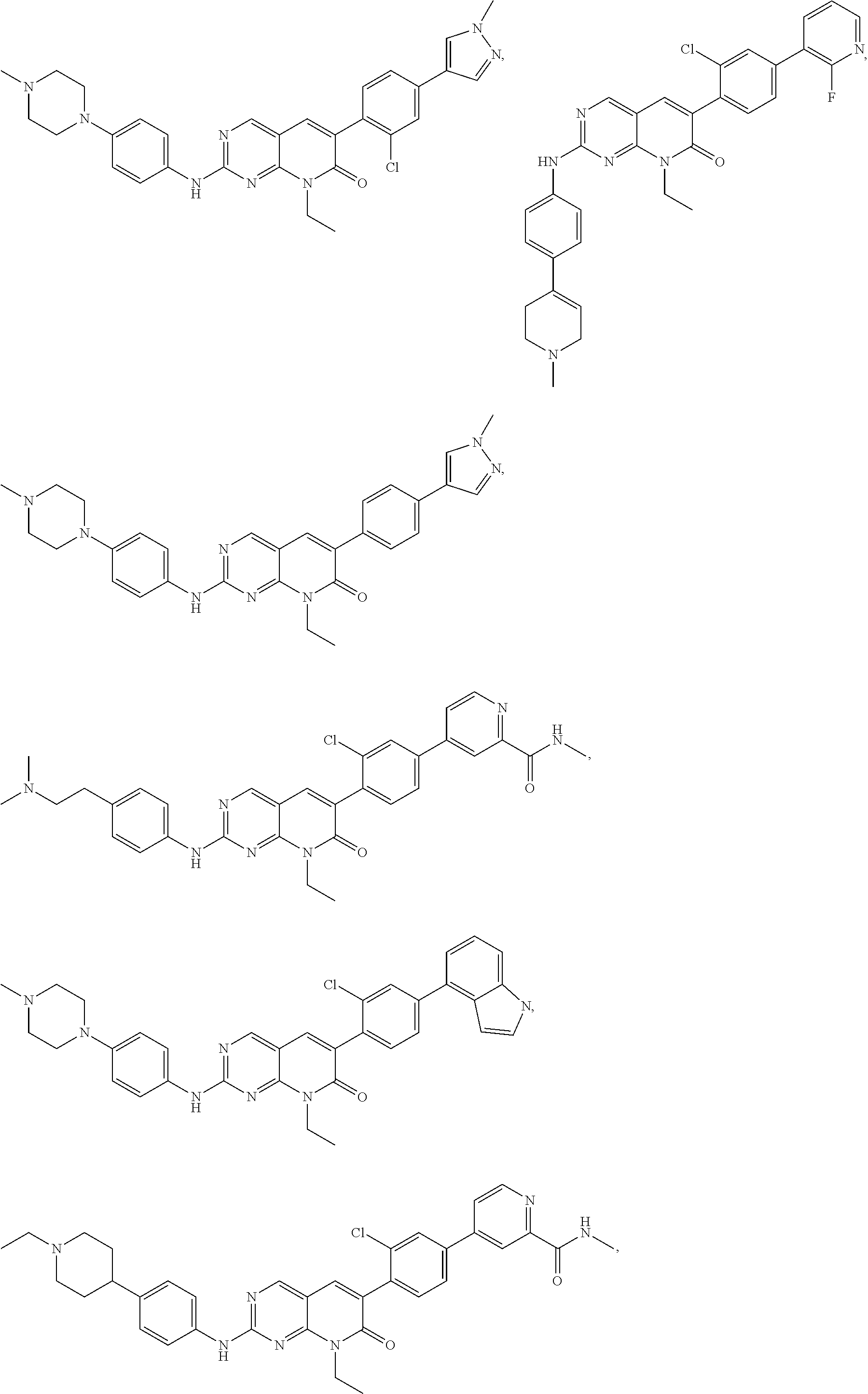

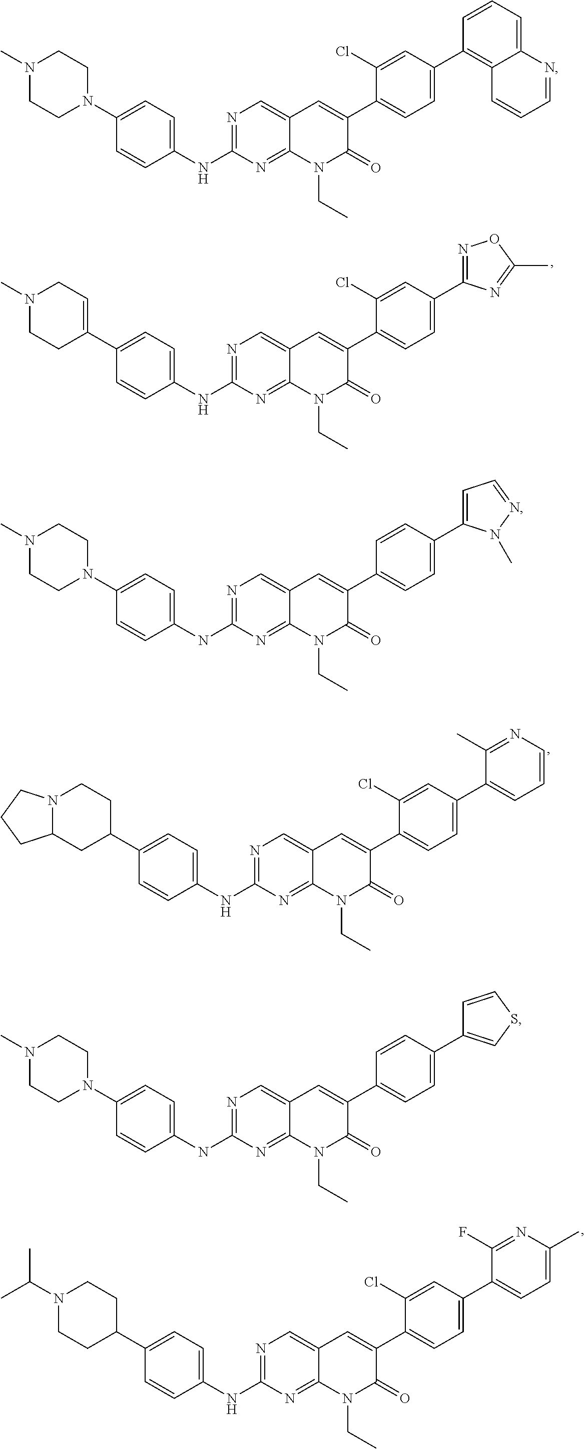

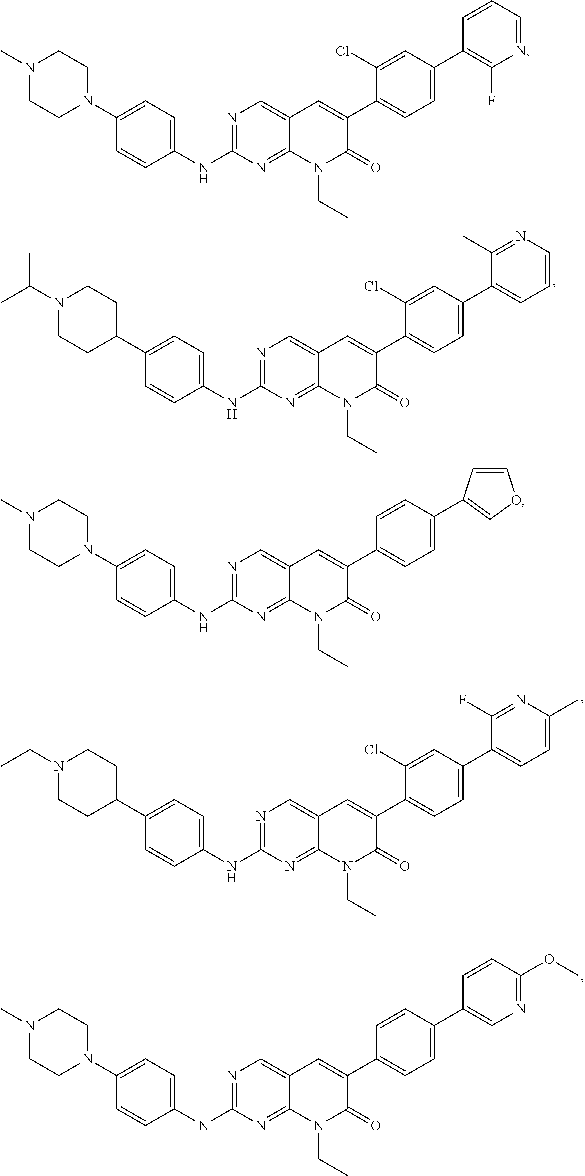

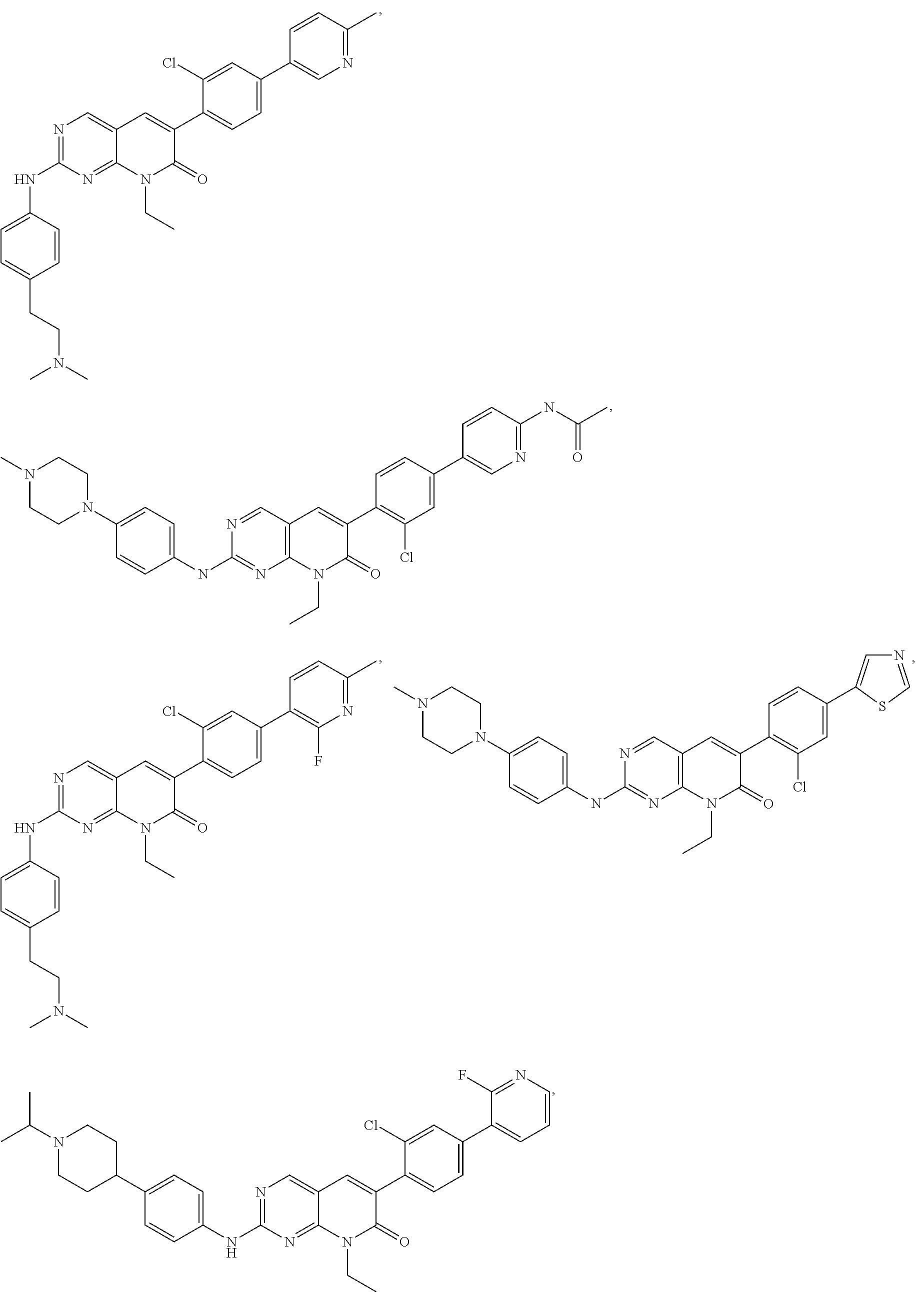

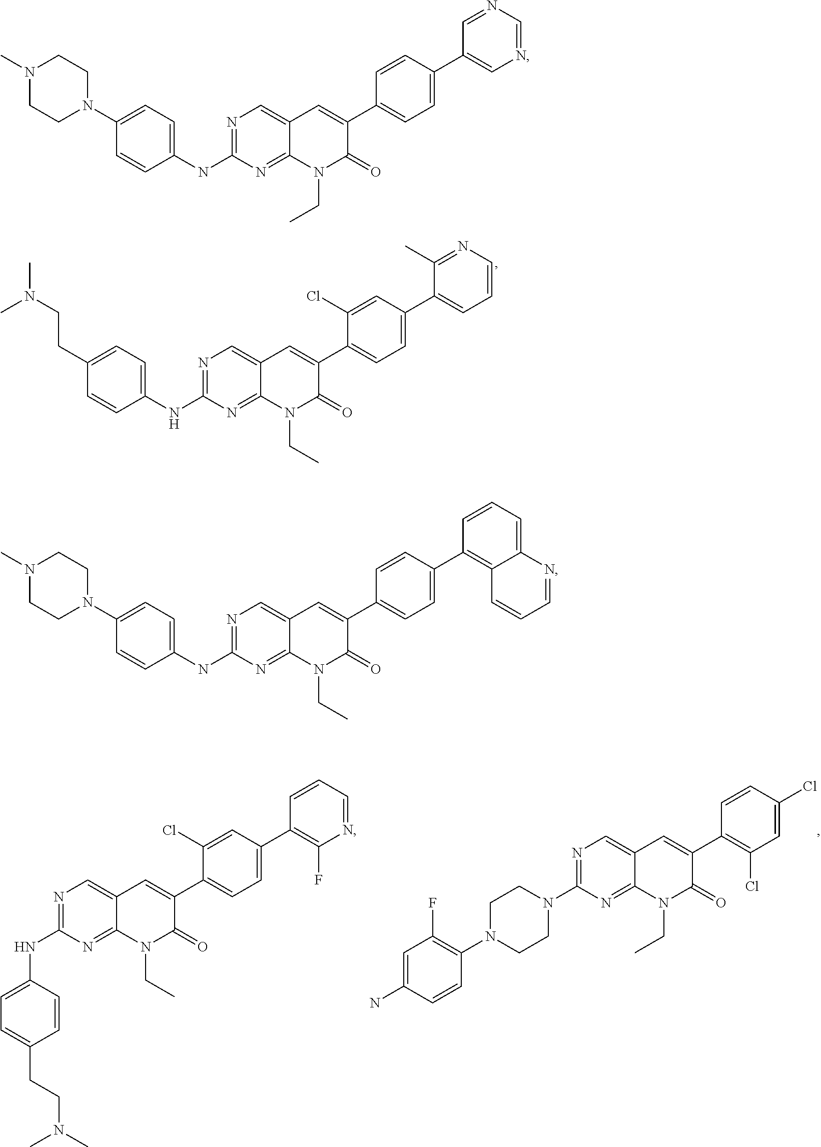 [SMILES: CCn1c(=O)c(-c2ccc(-c3cncnc3)cc2)cc2cnc(Nc3ccc(N4CCN(C)CC4)cc3)nc21 ; CCn1c(=O)c(-c2ccc(-c3cccnc3C)cc2Cl)cc2cnc(Nc3ccc(CCN(C)C)cc3)nc21 ; CCn1c(=O)c(-c2ccc(-c3cccc4ncccc34)cc2)cc2cnc(Nc3ccc(N4CCN(C)CC4)cc3)nc21 ; CCn1c(=O)c(-c2ccc(-c3cccnc3F)cc2Cl)cc2cnc(Nc3ccc(CCN(C)C)cc3)nc21 ; CCn1c(=O)c(-c2ccc(Cl)cc2Cl)cc2cnc(N3CCN(c4ccc(N)cc4F)CC3)nc21]

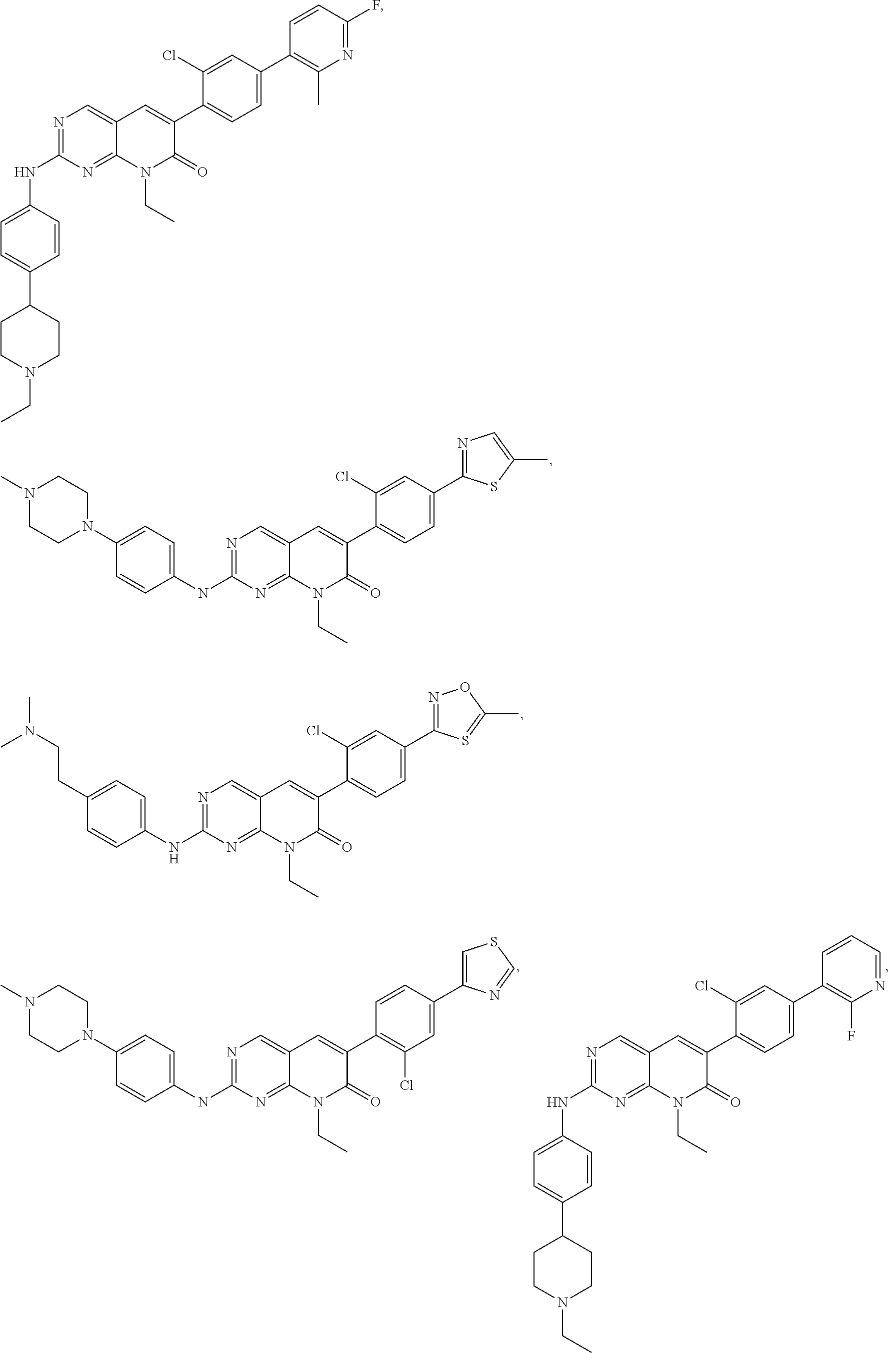

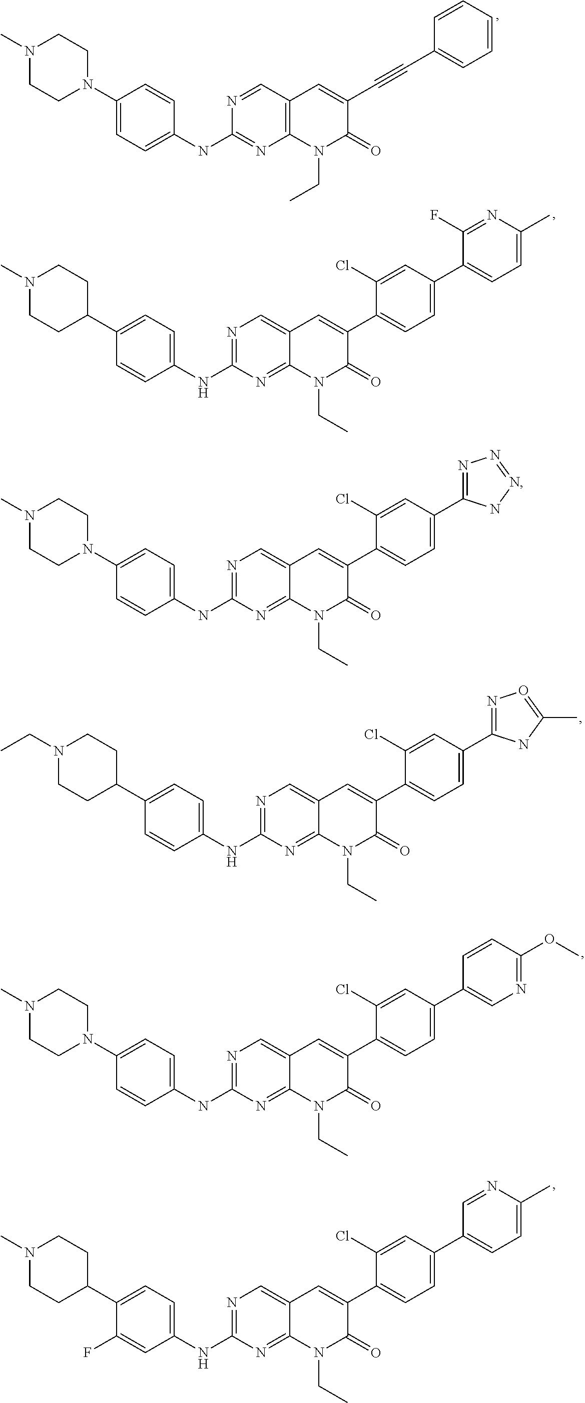

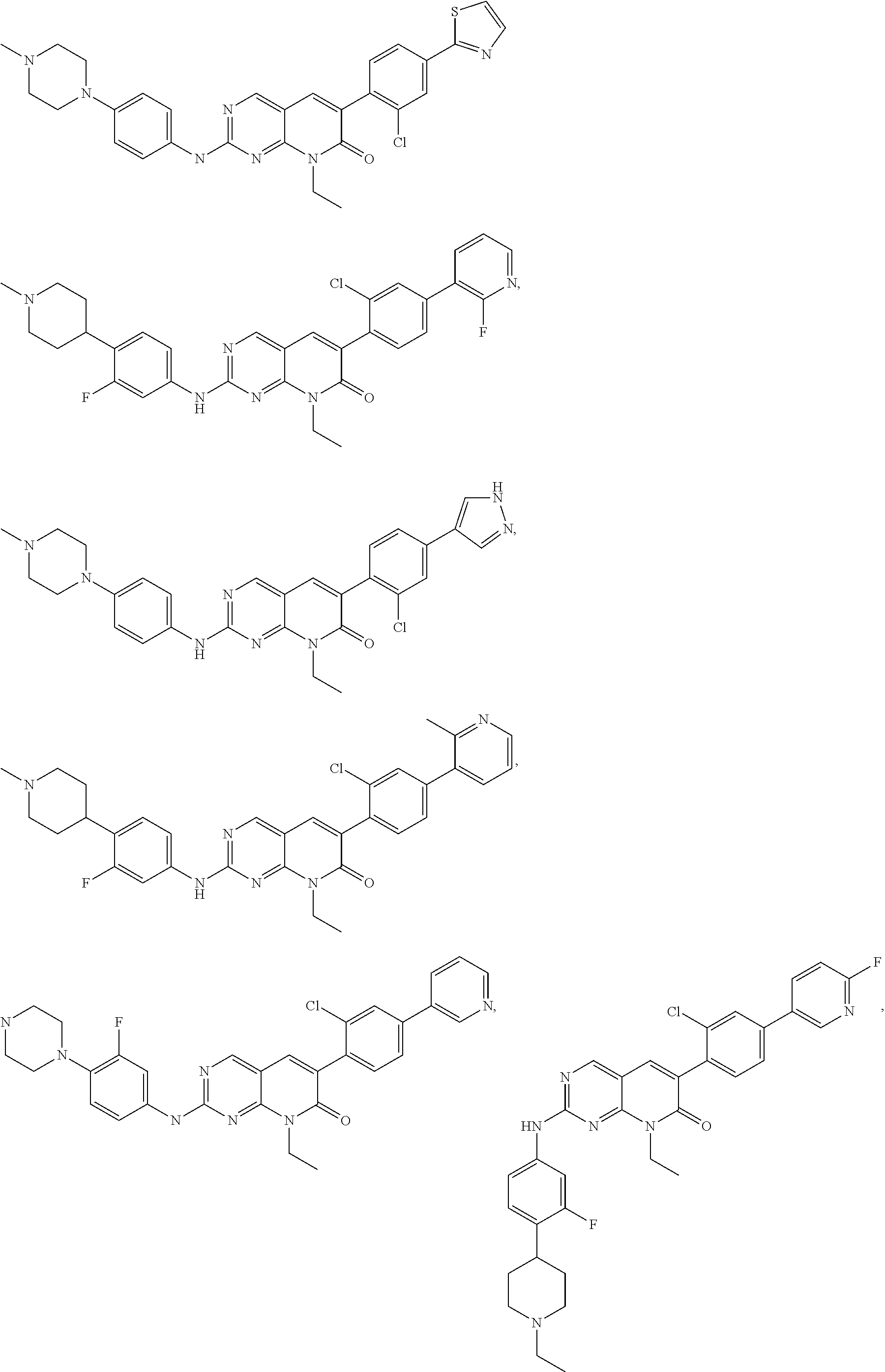 [SMILES: CCn1c(=O)c(-c2ccc(-c3nccs3)cc2Cl)cc2cnc(Nc3ccc(N4CCN(C)CC4)cc3)nc21 ; CCn1c(=O)c(-c2ccc(-c3cccnc3F)cc2Cl)cc2cnc(Nc3ccc(C4CCN(C)CC4)c(F)c3)nc21 ; CCn1c(=O)c(-c2ccc(-c3cn[nH]c3)cc2Cl)cc2cnc(Nc3ccc(N4CCN(C)CC4)cc3)nc21 ; CCn1c(=O)c(-c2ccc(-c3cccnc3C)cc2Cl)cc2cnc(Nc3ccc(C4CCN(C)CC4)c(F)c3)nc21 ; CCn1c(=O)c(-c2ccc(-c3cccnc3)cc2Cl)cc2cnc(Nc3ccc(N4CCNCC4)c(F)c3)nc21 ; CCN1CCC(c2ccc(Nc3ncc4cc(-c5ccc(-c6ccc(F)nc6)cc5Cl)c(=O)n(CC)c4n3)cc2F)CC1]

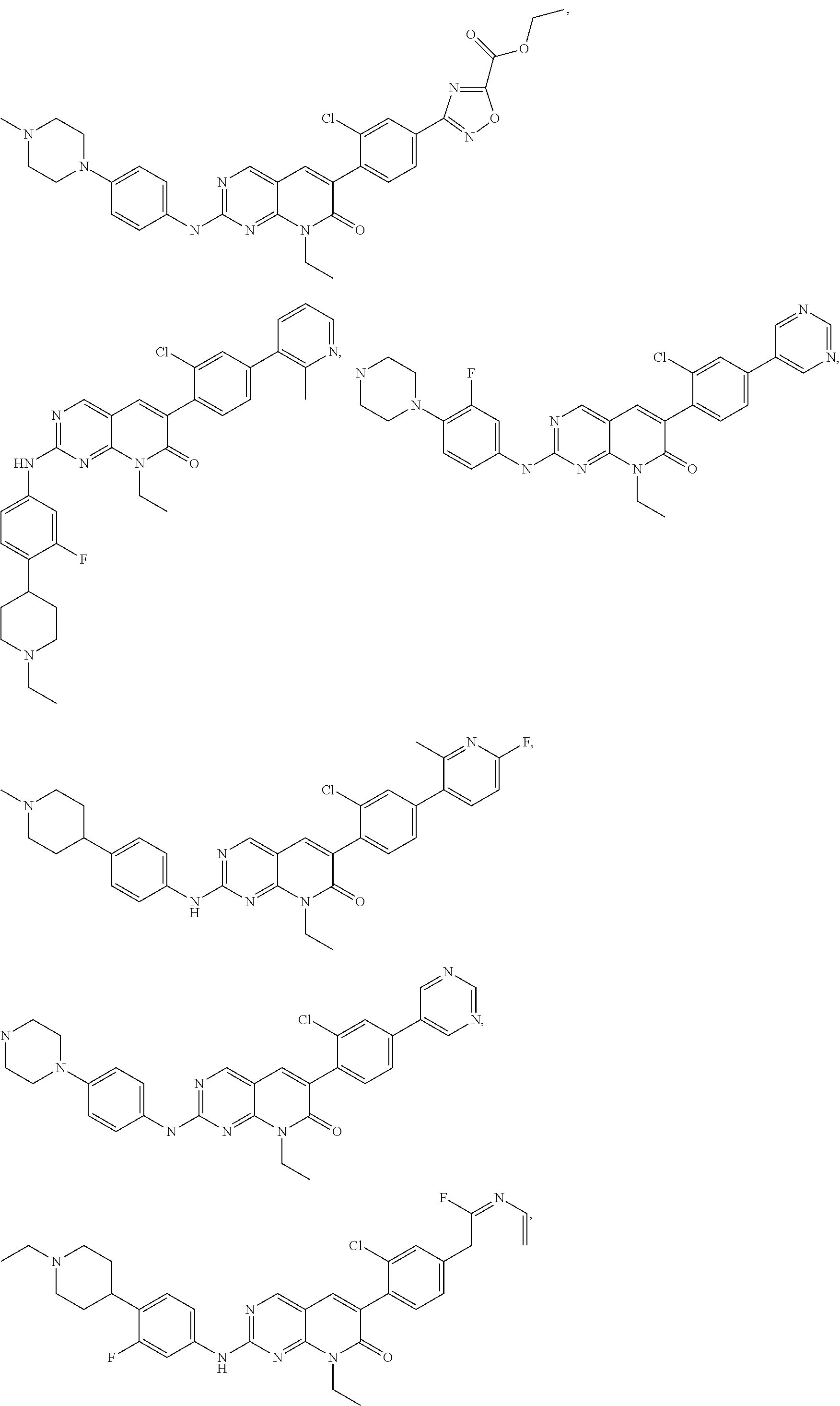

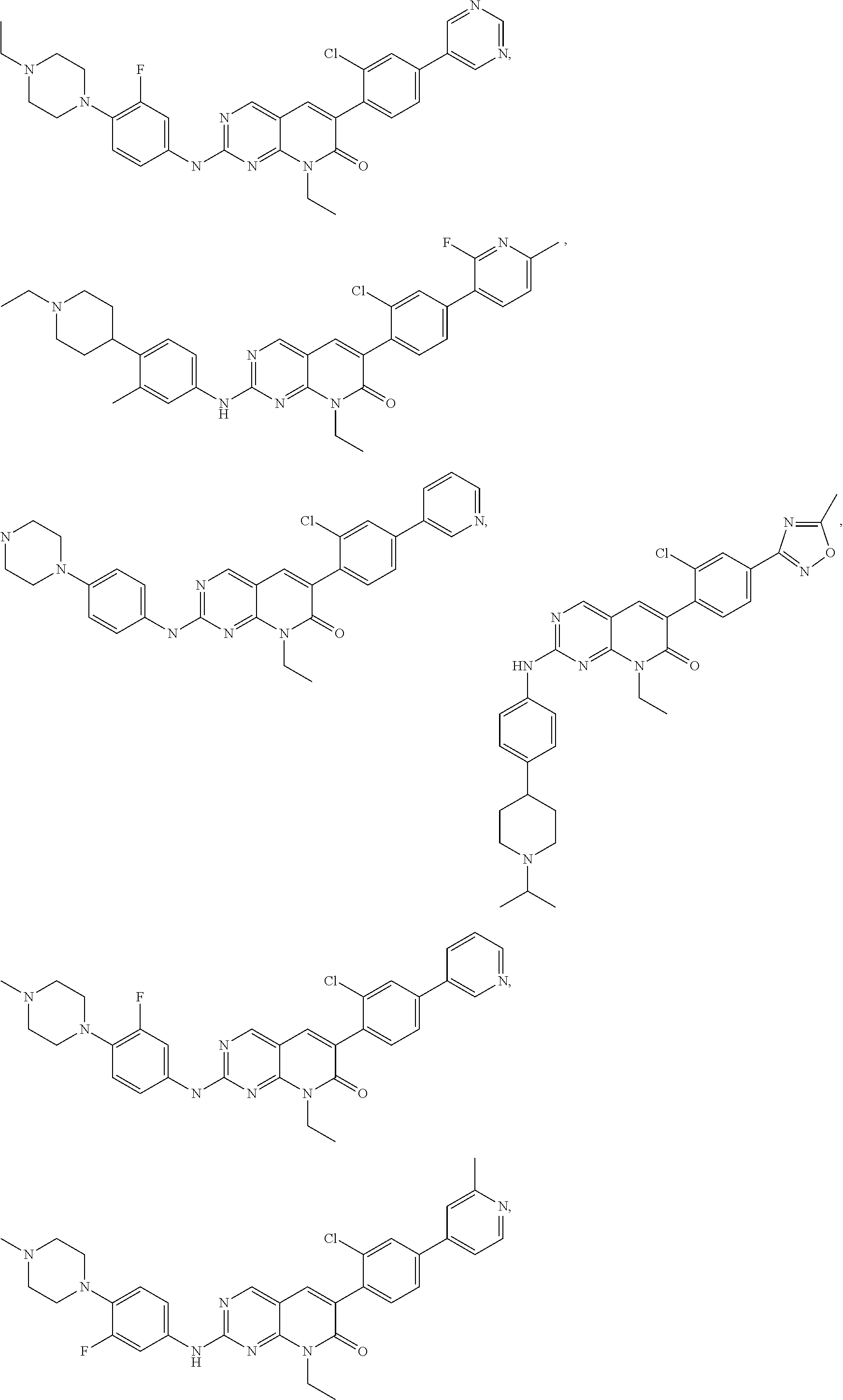

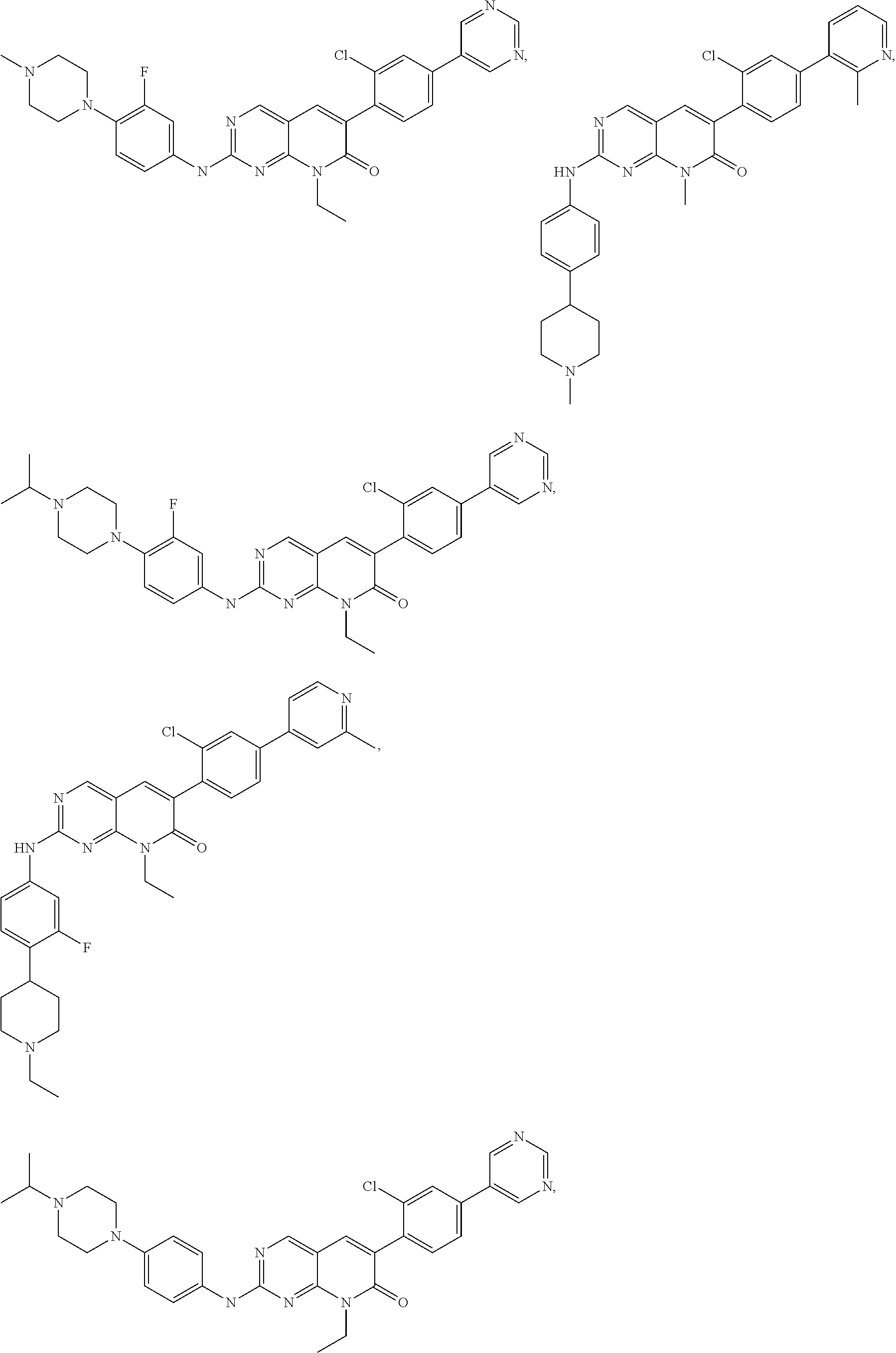 [SMILES: CCn1c(=O)c(-c2ccc(-c3cncnc3)cc2Cl)cc2cnc(Nc3ccc(N4CCN(C)CC4)c(F)c3)nc21 ; Cc1ncccc1-c1ccc(-c2cc3cnc(Nc4ccc(C5CCN(C)CC5)cc4)nc3n(C)c2=O)c(Cl)c1 ; CCn1c(=O)c(-c2ccc(-c3cncnc3)cc2Cl)cc2cnc(Nc3ccc(N4CCN(C(C)C)CC4)c(F)c3)nc21 ; CCN1CCC(c2ccc(Nc3ncc4cc(-c5ccc(-c6ccnc(C)c6)cc5Cl)c(=O)n(CC)c4n3)cc2F)CC1 ; CCn1c(=O)c(-c2ccc(-c3cncnc3)cc2Cl)cc2cnc(Nc3ccc(N4CCN(C(C)C)CC4)cc3)nc21]

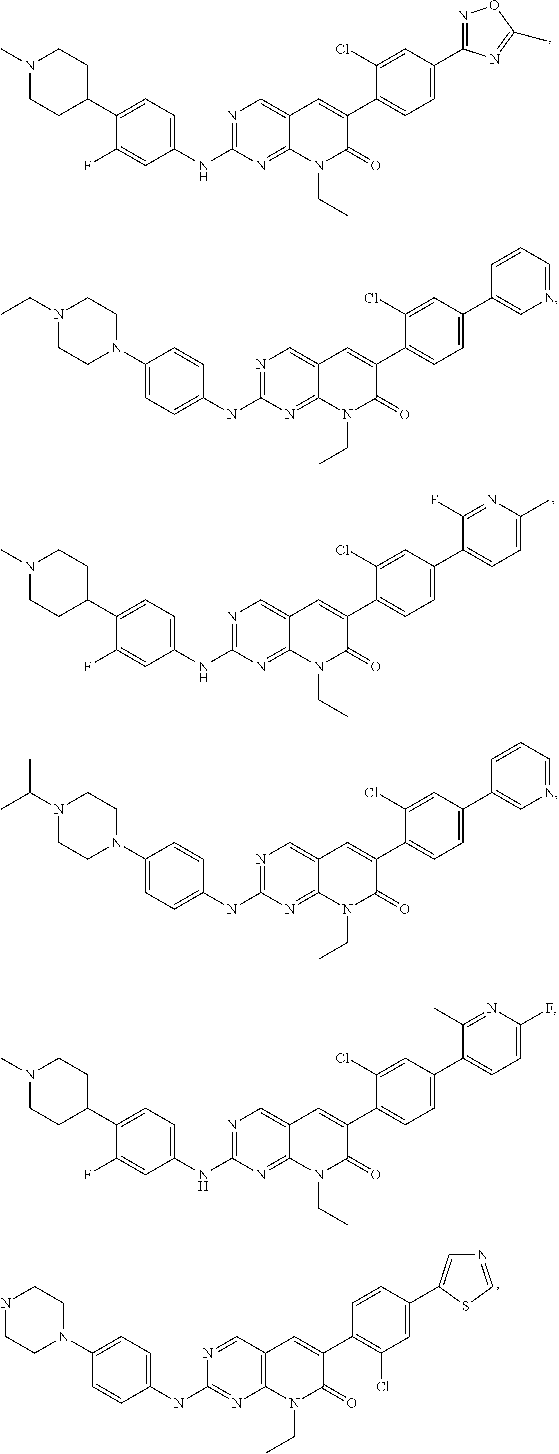

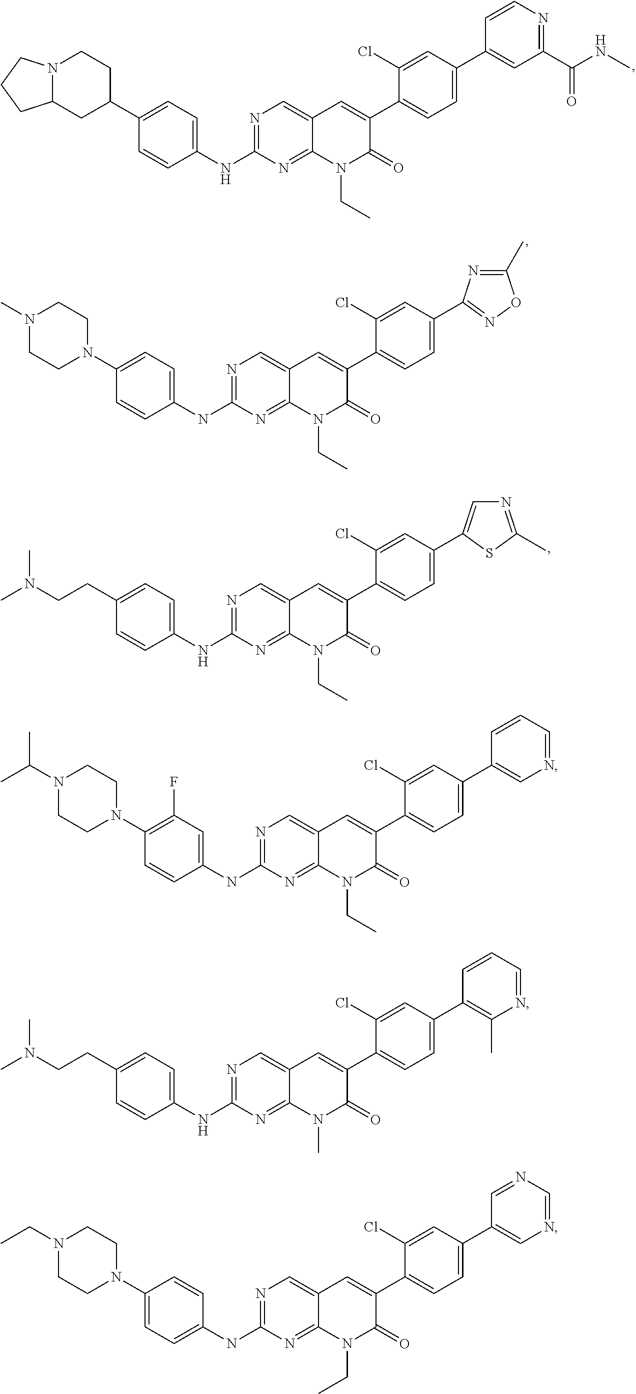

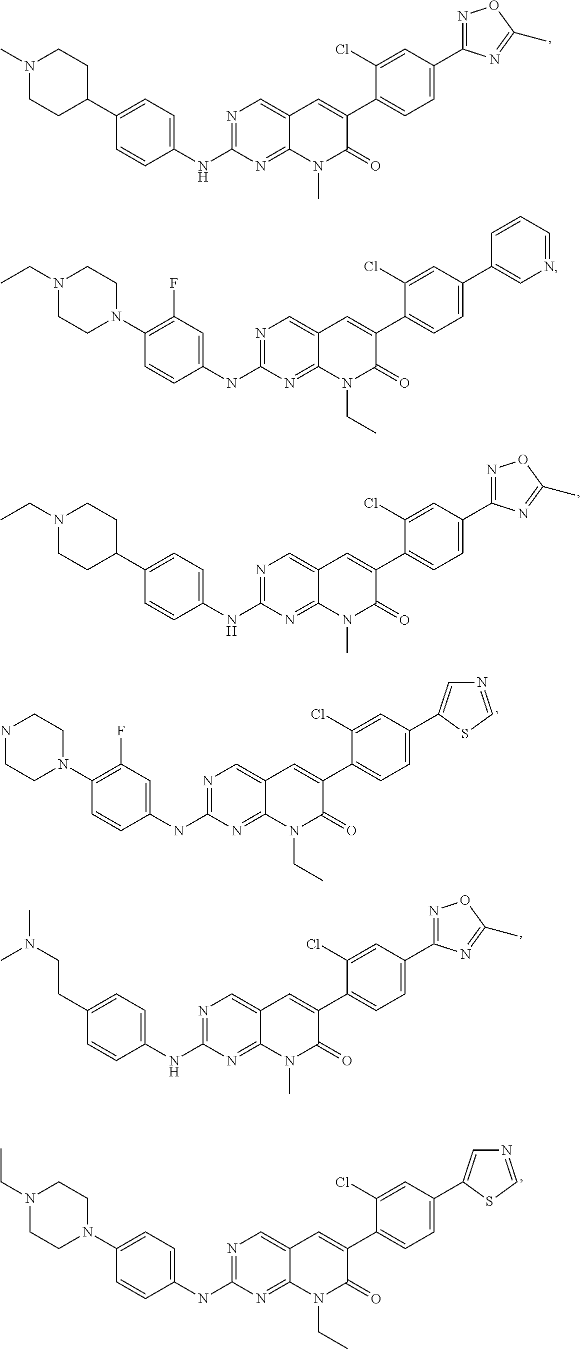

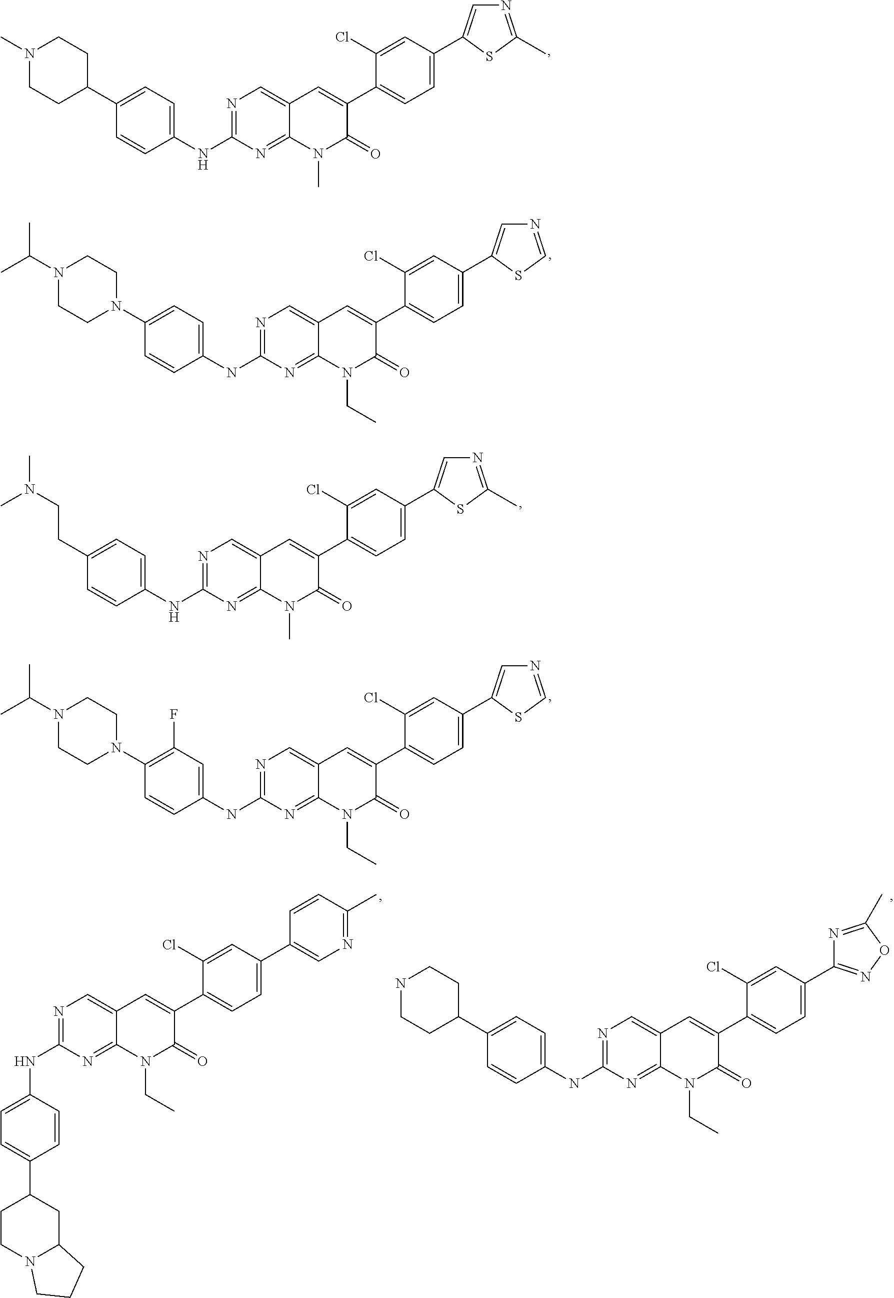

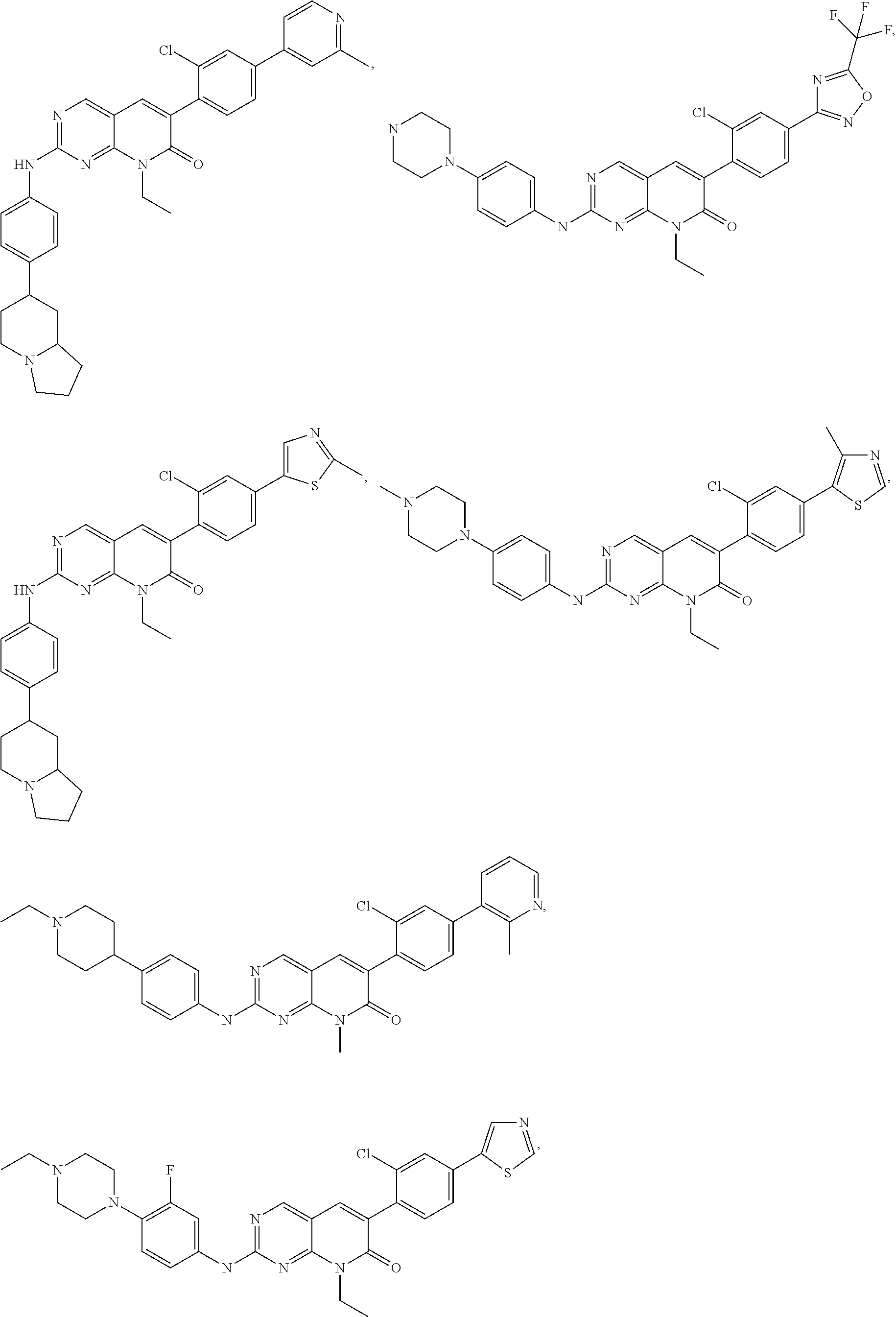

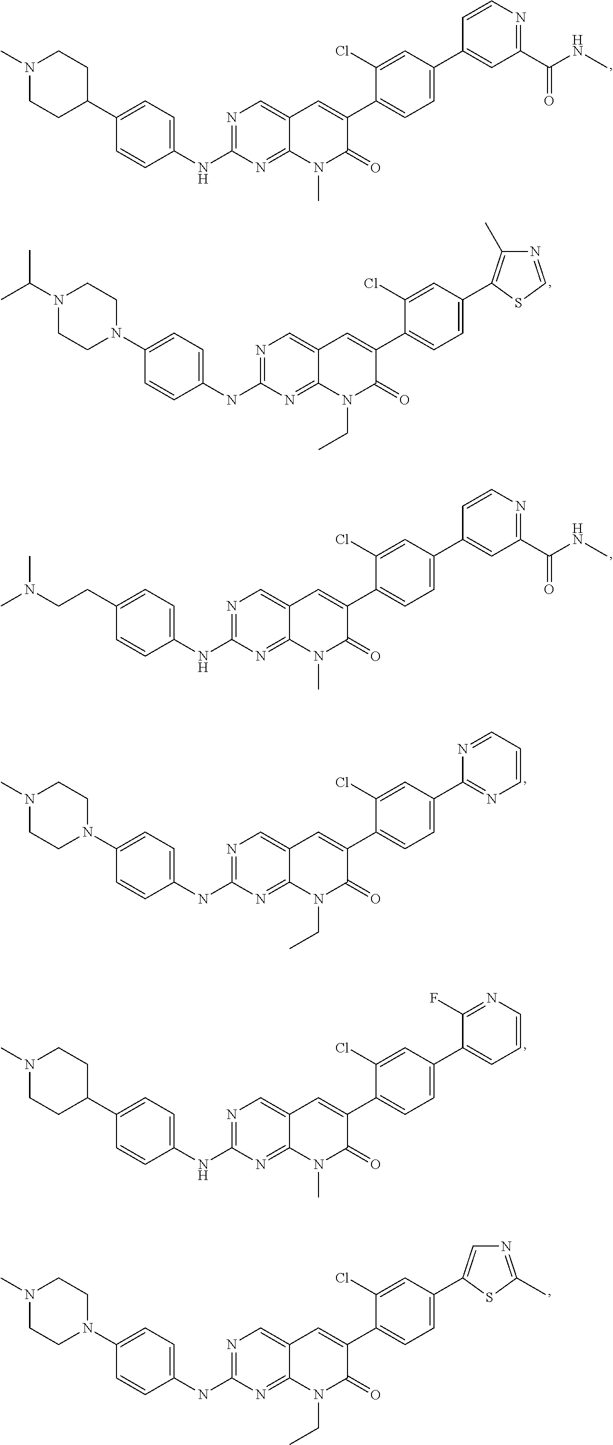

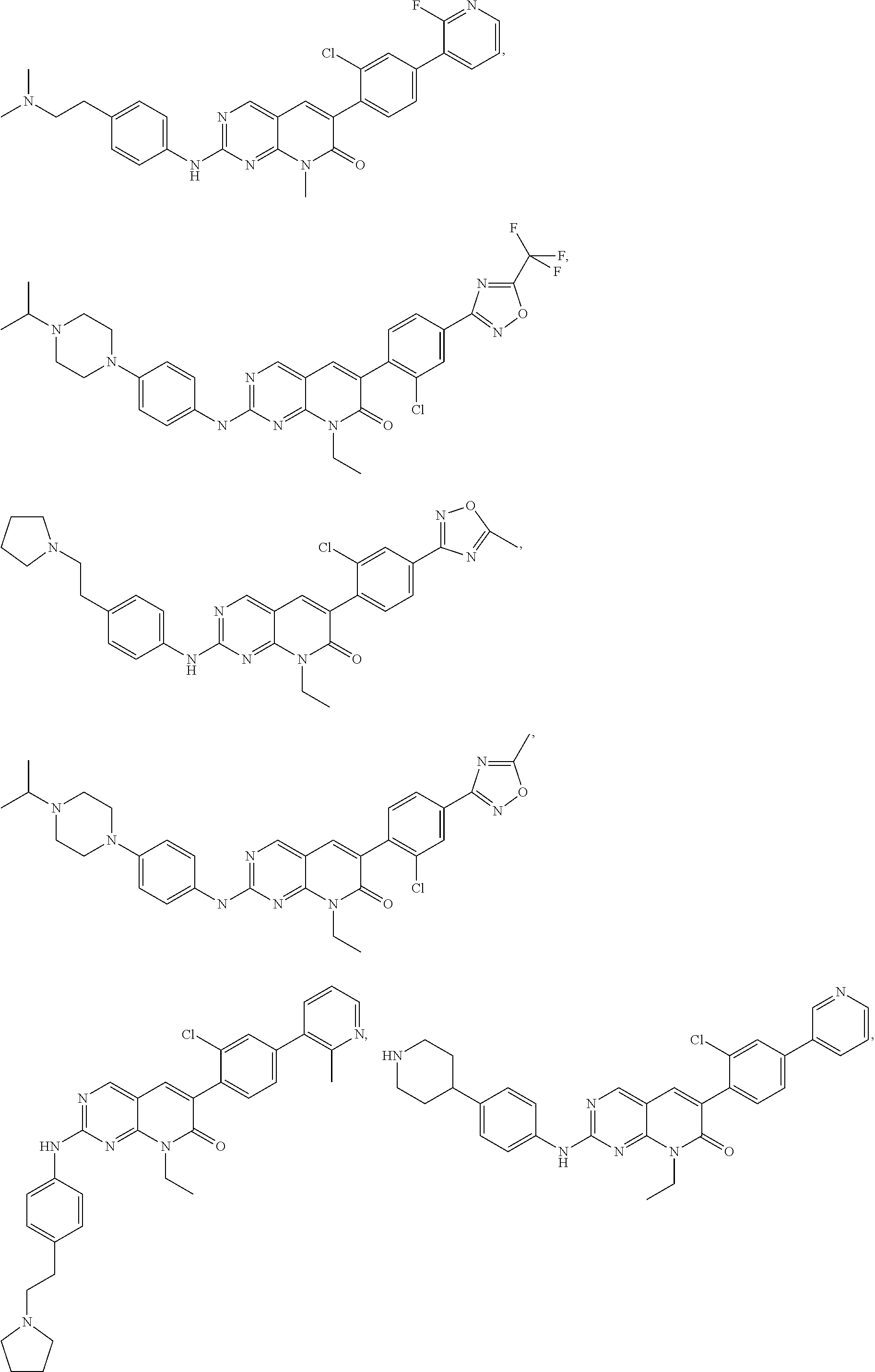

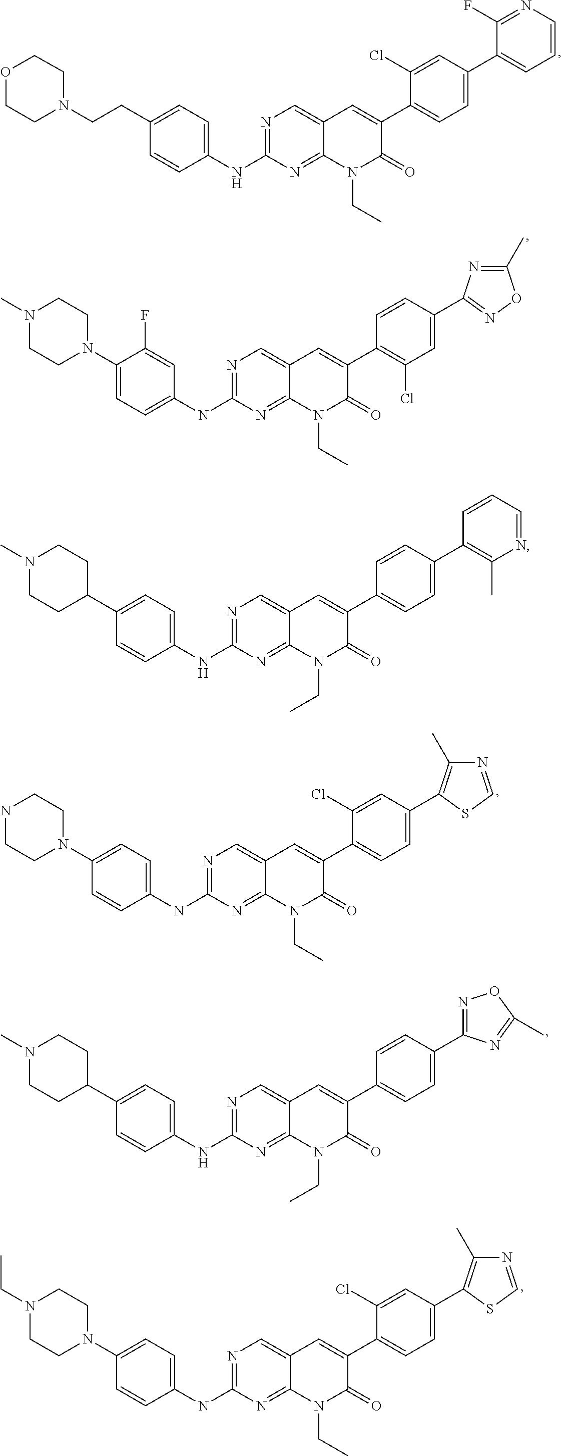

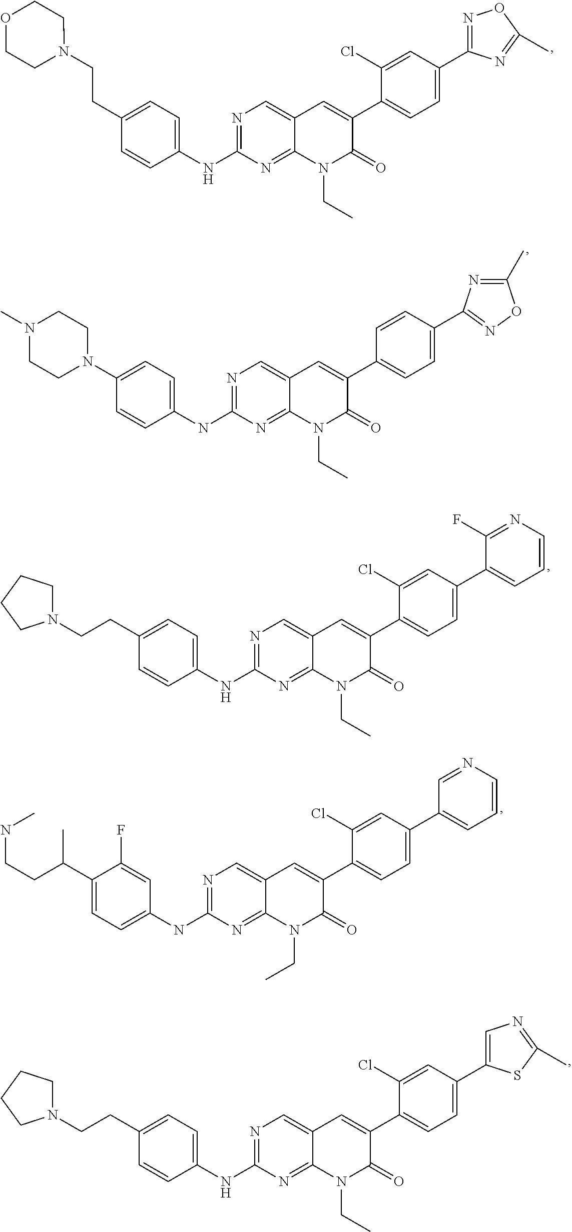

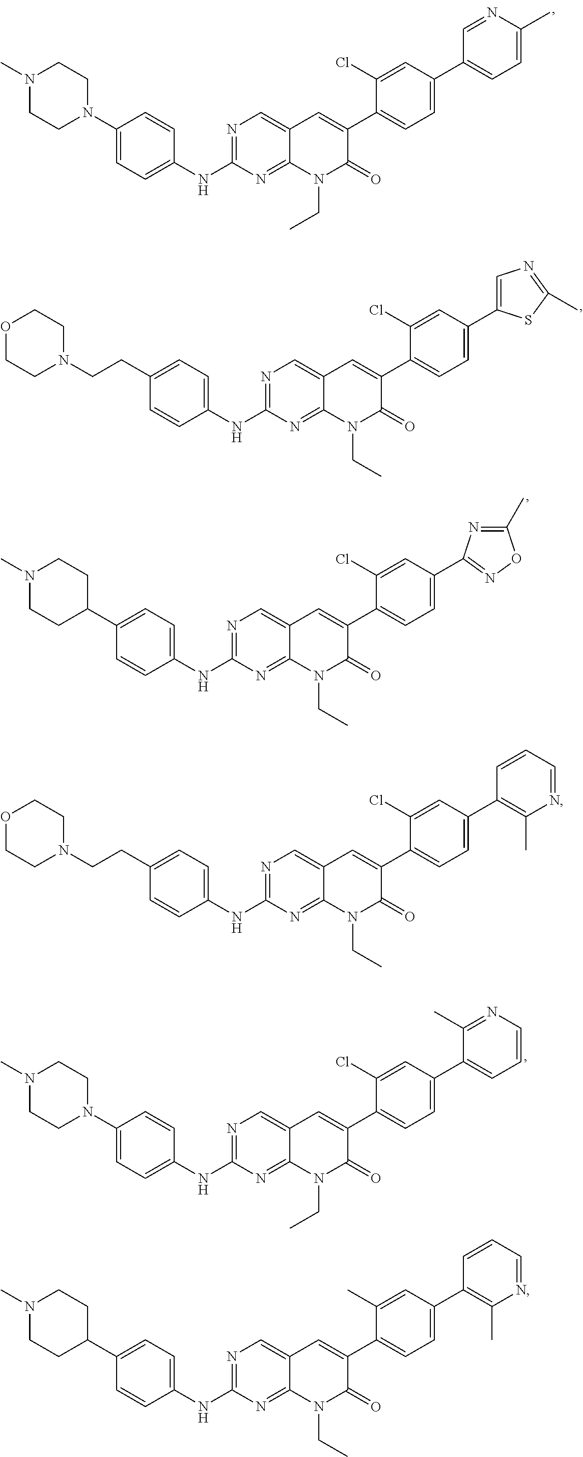

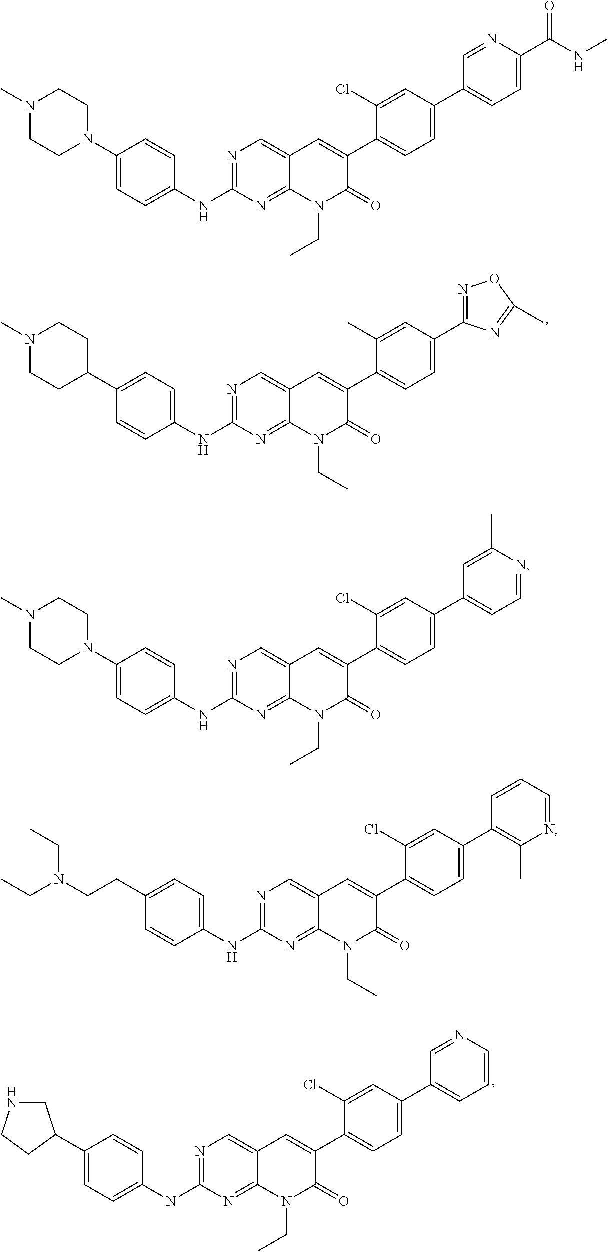

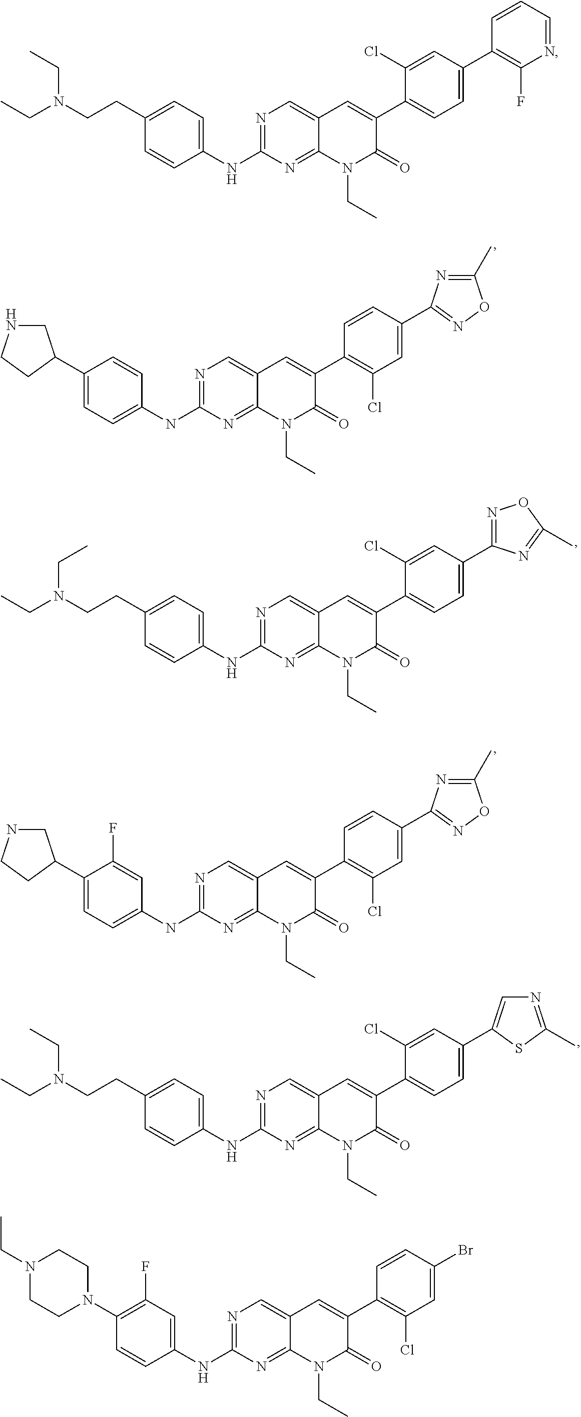

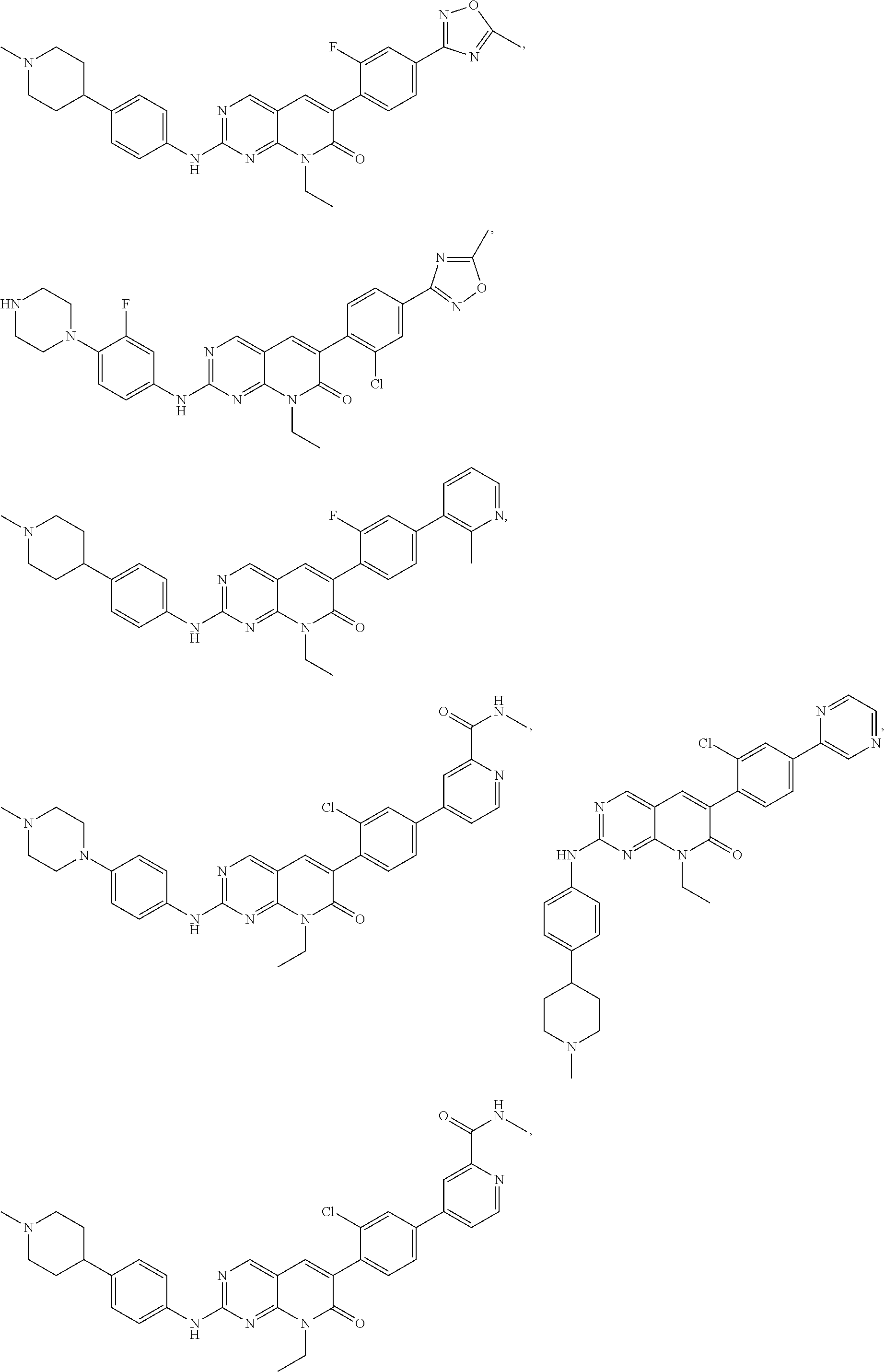

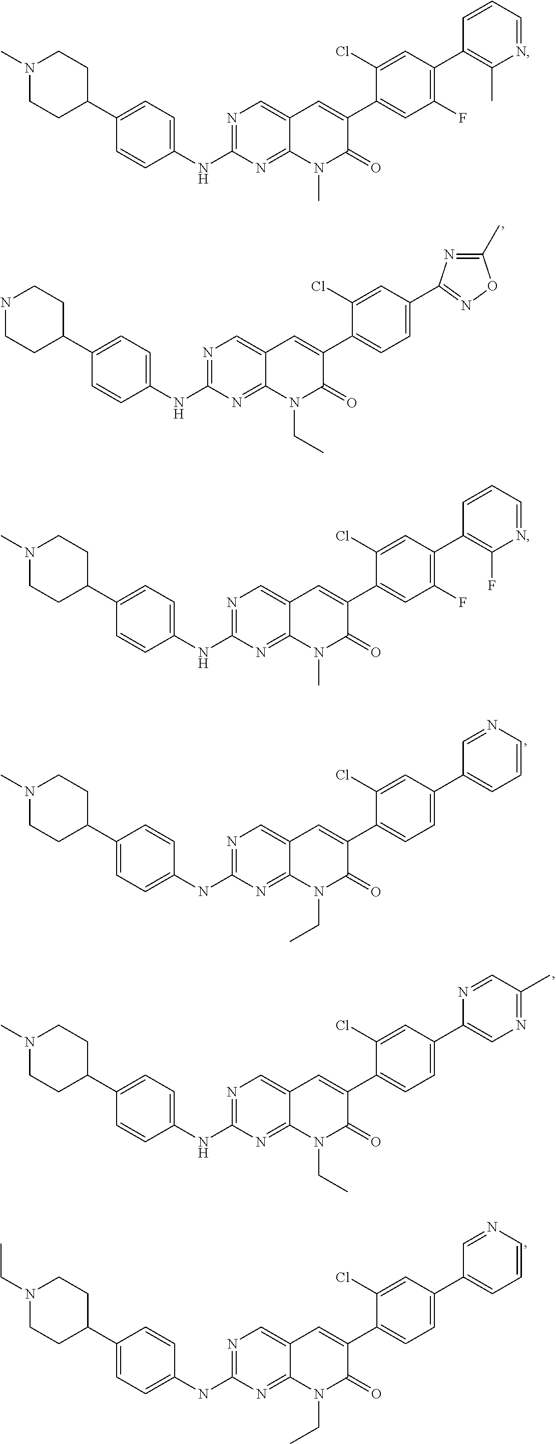

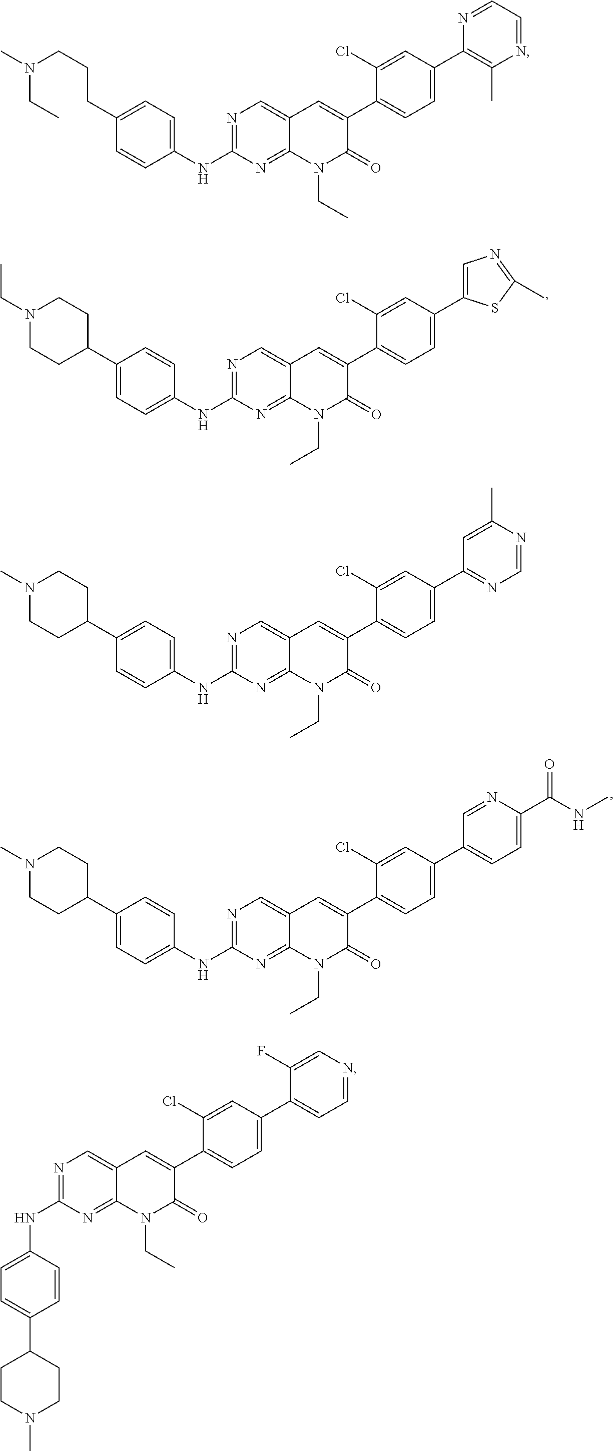

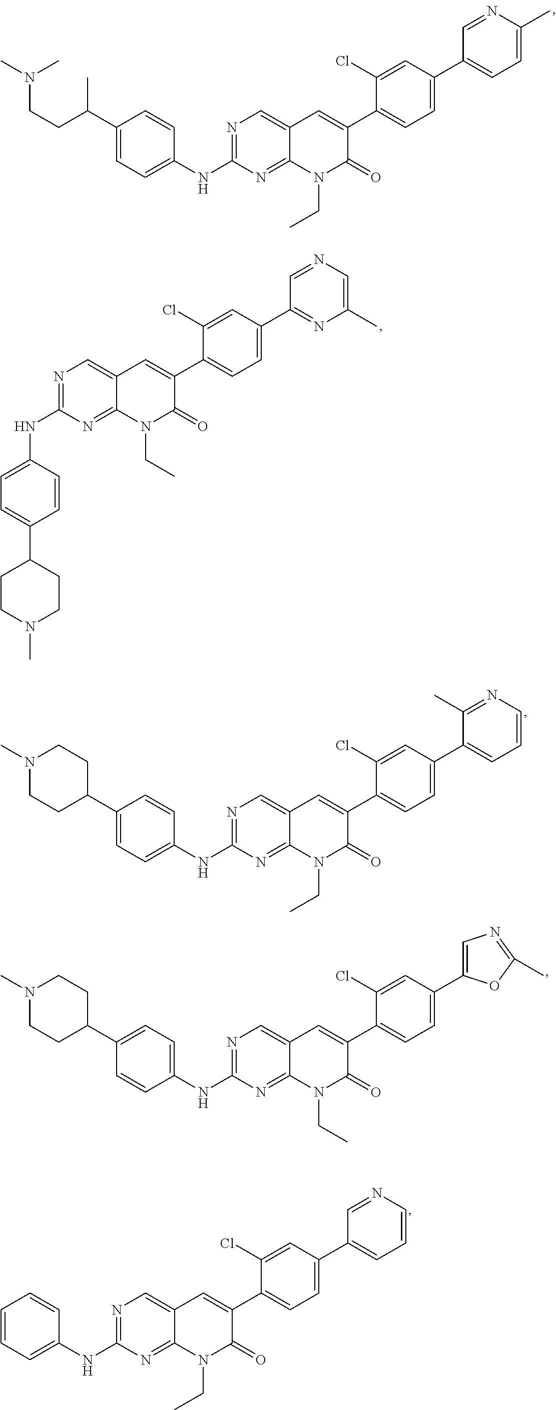

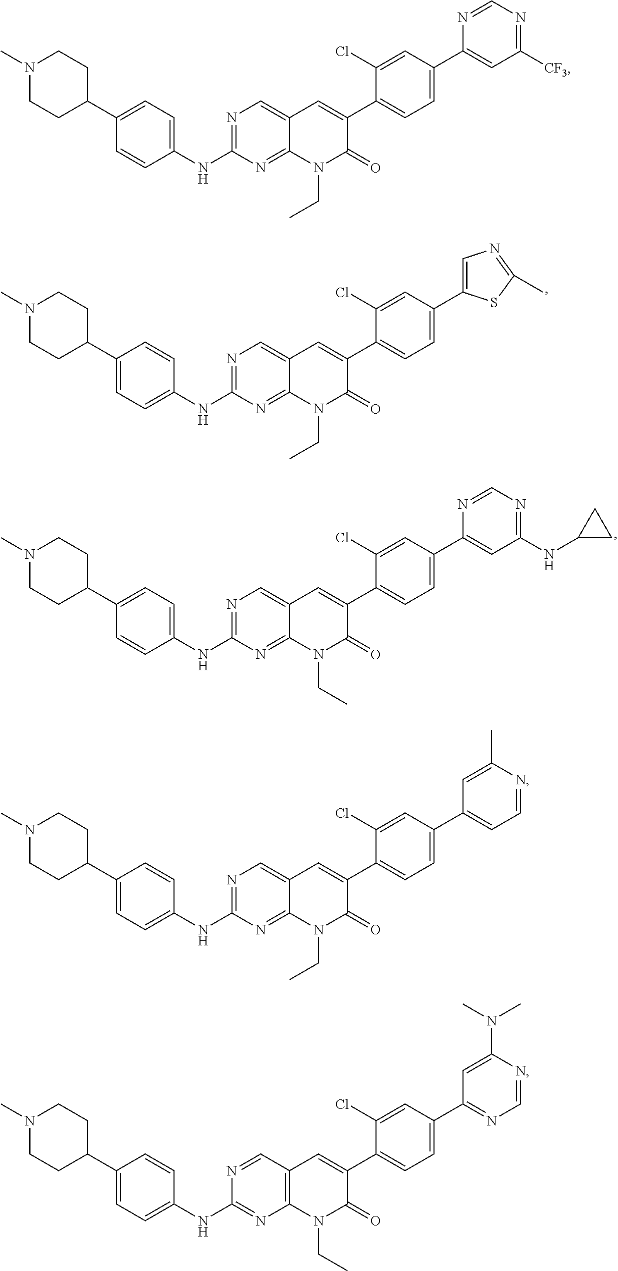

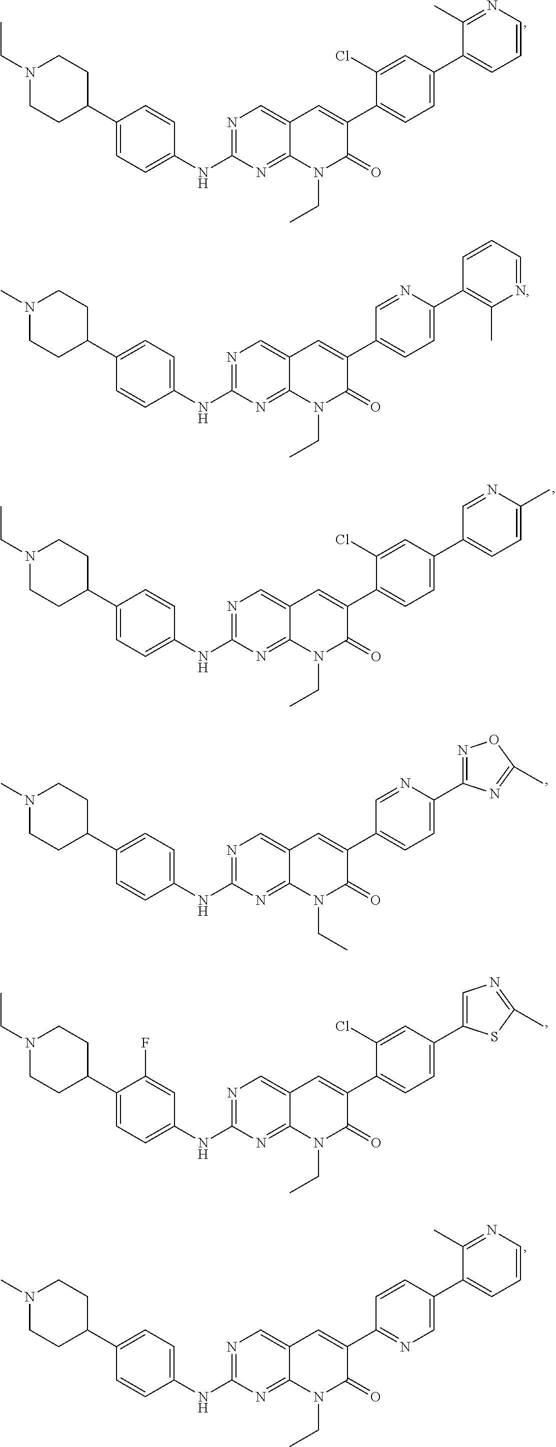

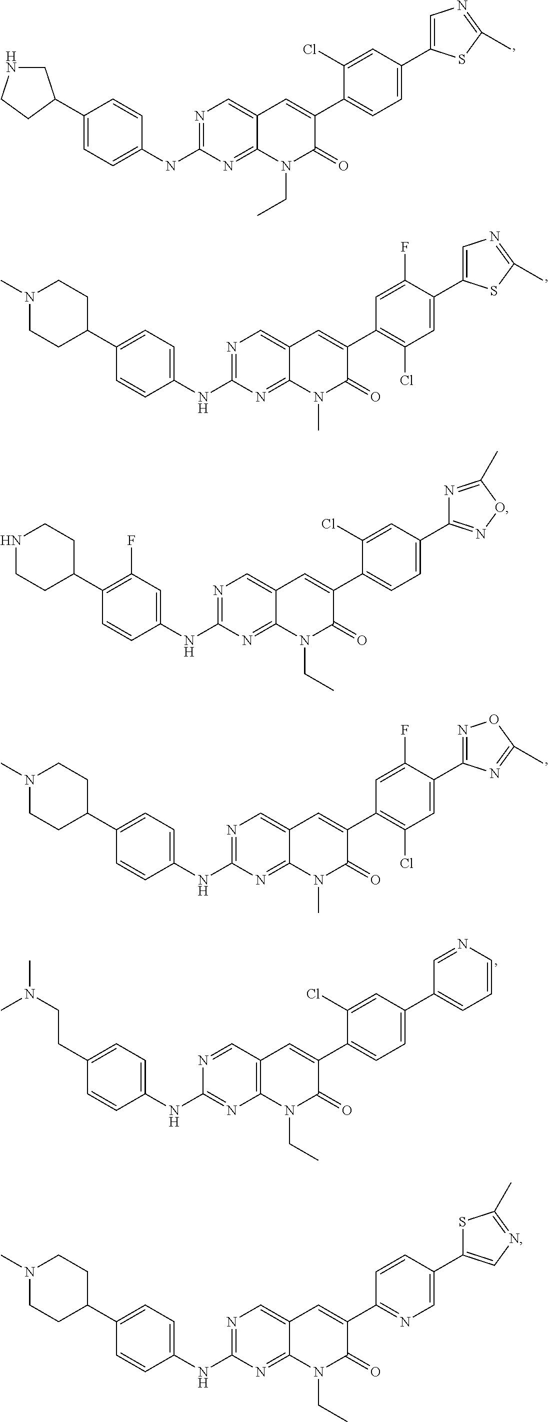

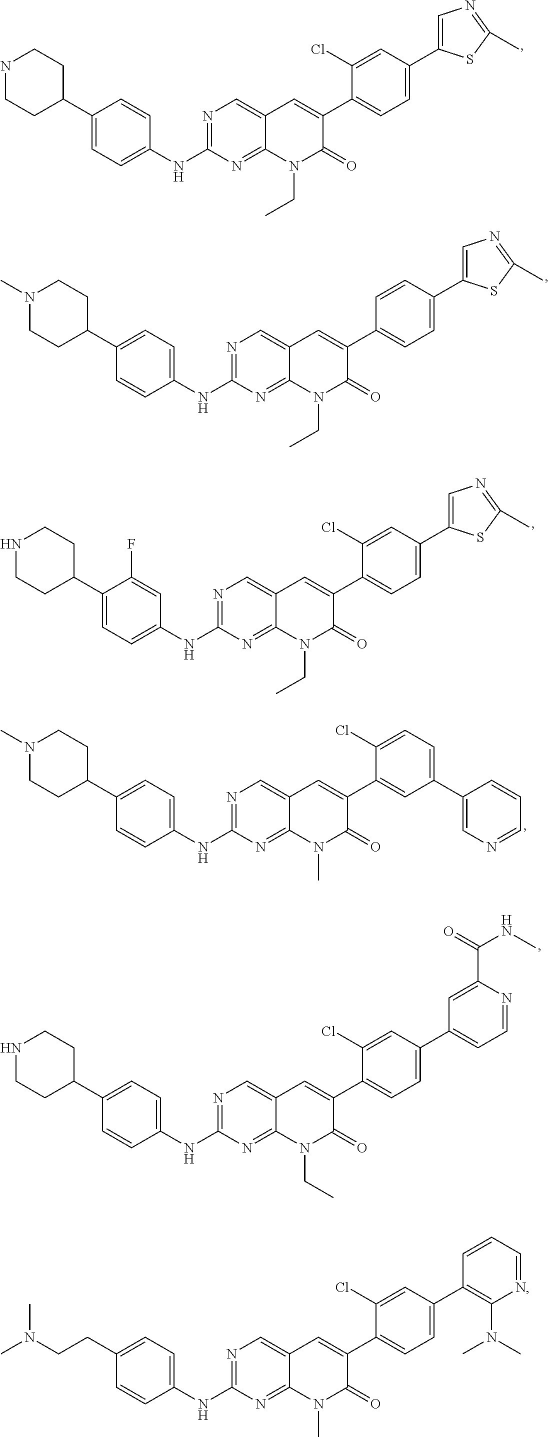

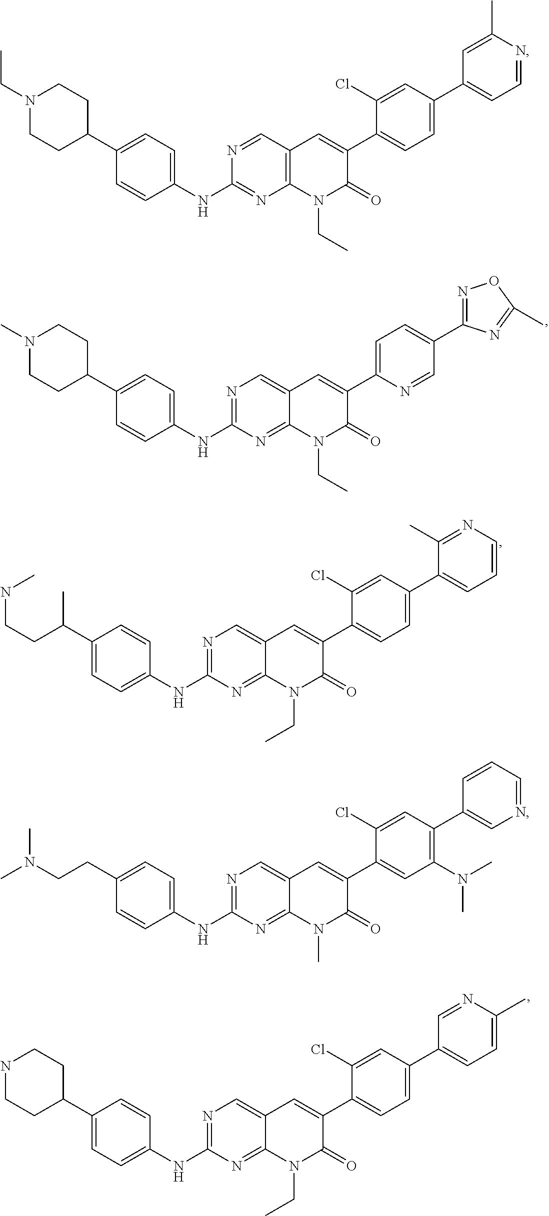

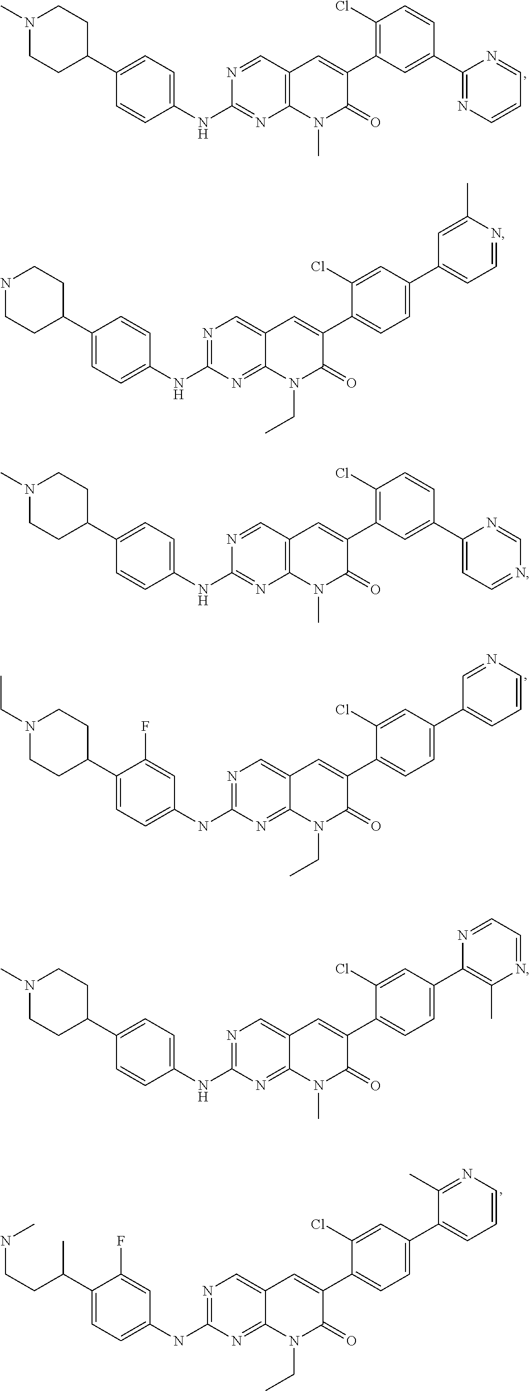 [SMILES: CN1CCC(c2ccc(Nc3ncc4cc(-c5cc(-c6ncccn6)ccc5Cl)c(=O)n(C)c4n3)cc2)CC1 ; CCn1c(=O)c(-c2ccc(-c3ccnc(C)c3)cc2Cl)cc2cnc(Nc3ccc(C4CCNCC4)cc3)nc21 ; CN1CCC(c2ccc(Nc3ncc4cc(-c5cc(-c6ccncn6)ccc5Cl)c(=O)n(C)c4n3)cc2)CC1 ; CCN1CCC(c2ccc(Nc3ncc4cc(-c5ccc(-c6cccnc6)cc5Cl)c(=O)n(CC)c4n3)cc2F)CC1 ; Cc1nccnc1-c1ccc(-c2cc3cnc(Nc4ccc(C5CCN(C)CC5)cc4)nc3n(C)c2=O)c(Cl)c1 ; CCn1c(=O)c(-c2ccc(-c3cccnc3C)cc2Cl)cc2cnc(Nc3ccc(C(C)CCNC)c(F)c3)nc21]

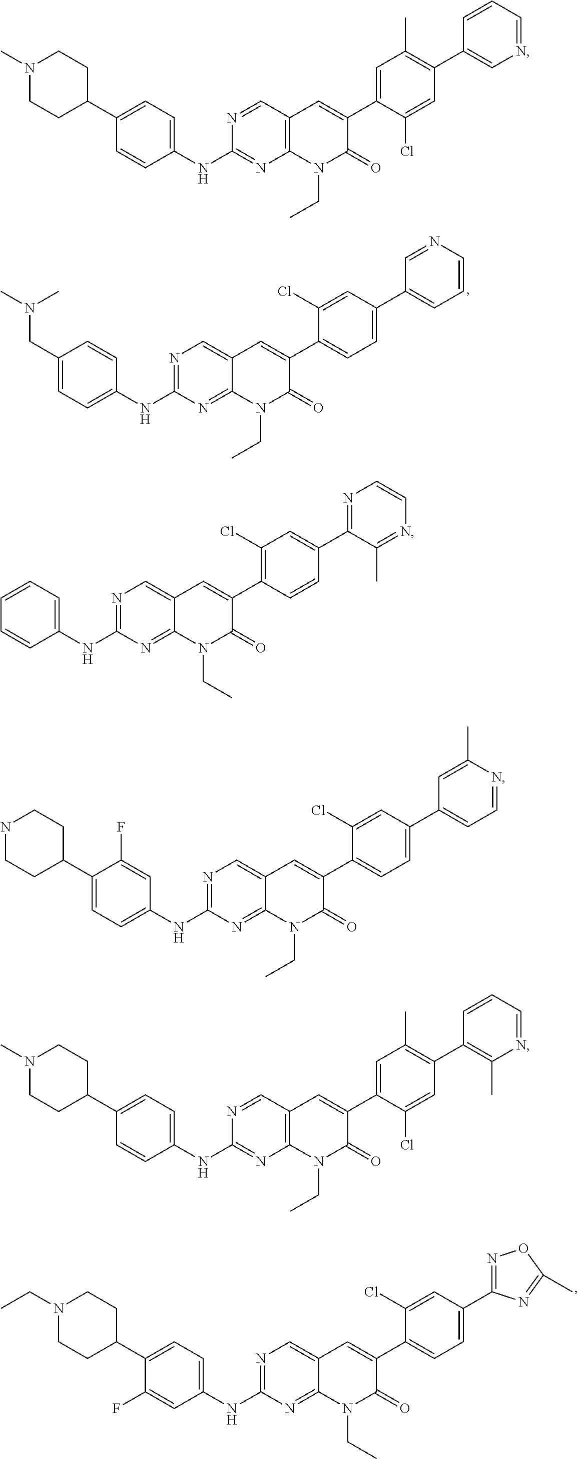 [SMILES: CCn1c(=O)c(-c2cc(C)c(-c3cccnc3)cc2Cl)cc2cnc(Nc3ccc(C4CCN(C)CC4)cc3)nc21 ; CCn1c(=O)c(-c2ccc(-c3cccnc3)cc2Cl)cc2cnc(Nc3ccc(CN(C)C)cc3)nc21 ; CCn1c(=O)c(-c2ccc(-c3nccnc3C)cc2Cl)cc2cnc(Nc3ccccc3)nc21 ; CCn1c(=O)c(-c2ccc(-c3ccnc(C)c3)cc2Cl)cc2cnc(Nc3ccc(C4CCNCC4)c(F)c3)nc21 ; CCn1c(=O)c(-c2cc(C)c(-c3cccnc3C)cc2Cl)cc2cnc(Nc3ccc(C4CCN(C)CC4)cc3)nc21 ; CCN1CCC(c2ccc(Nc3ncc4cc(-c5ccc(-c6noc(C)n6)cc5Cl)c(=O)n(CC)c4n3)cc2F)CC1]

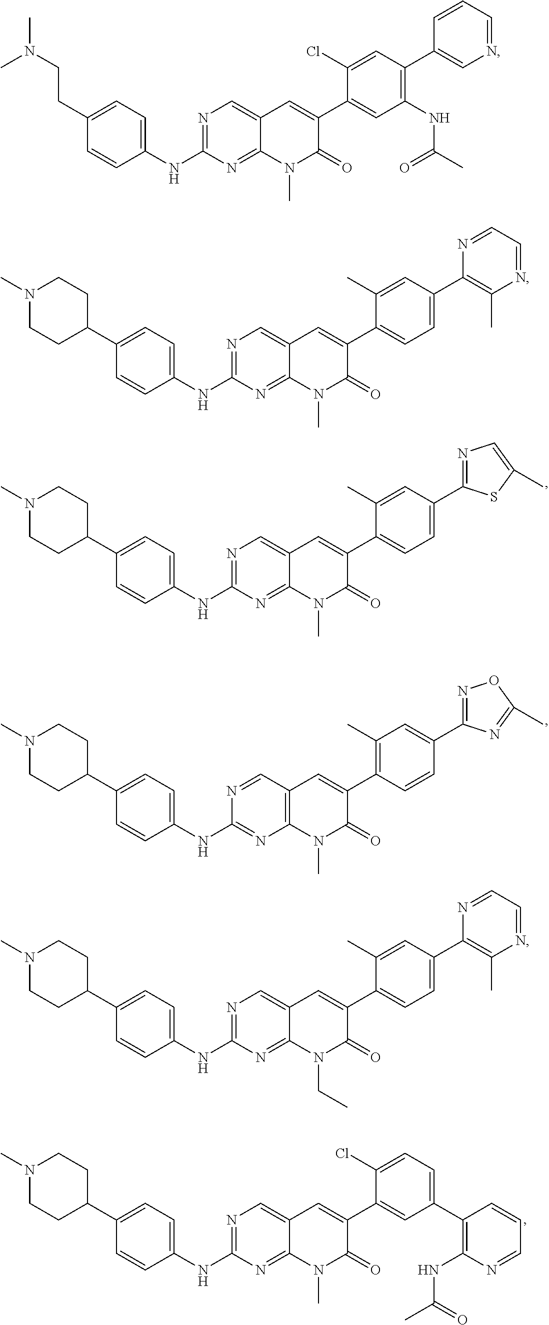

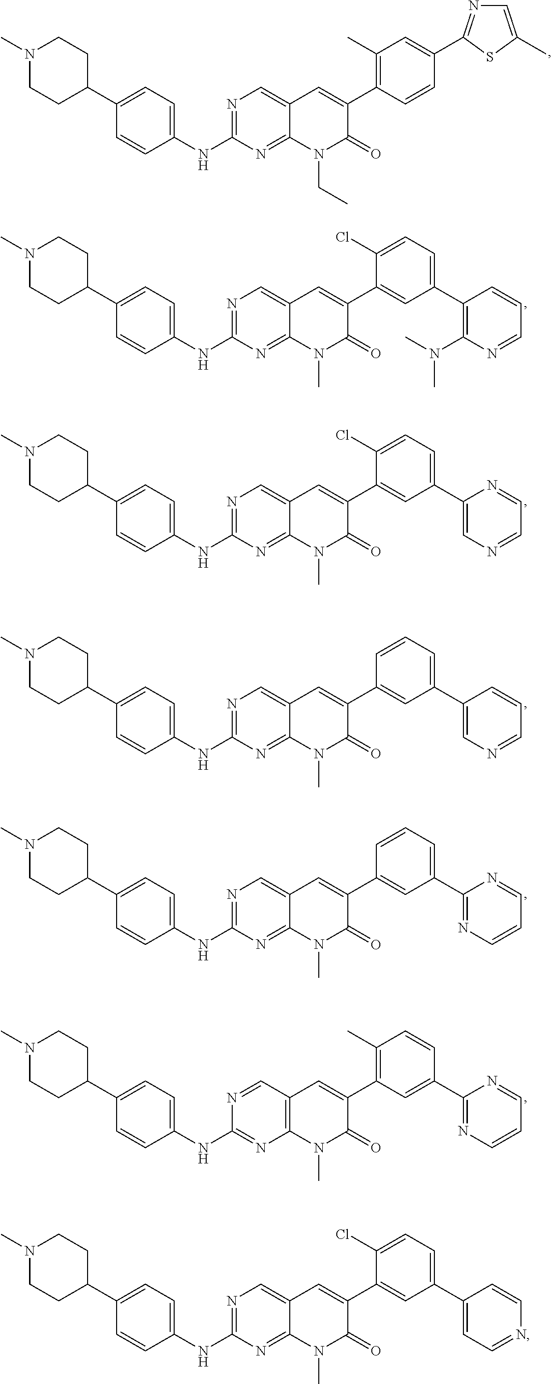

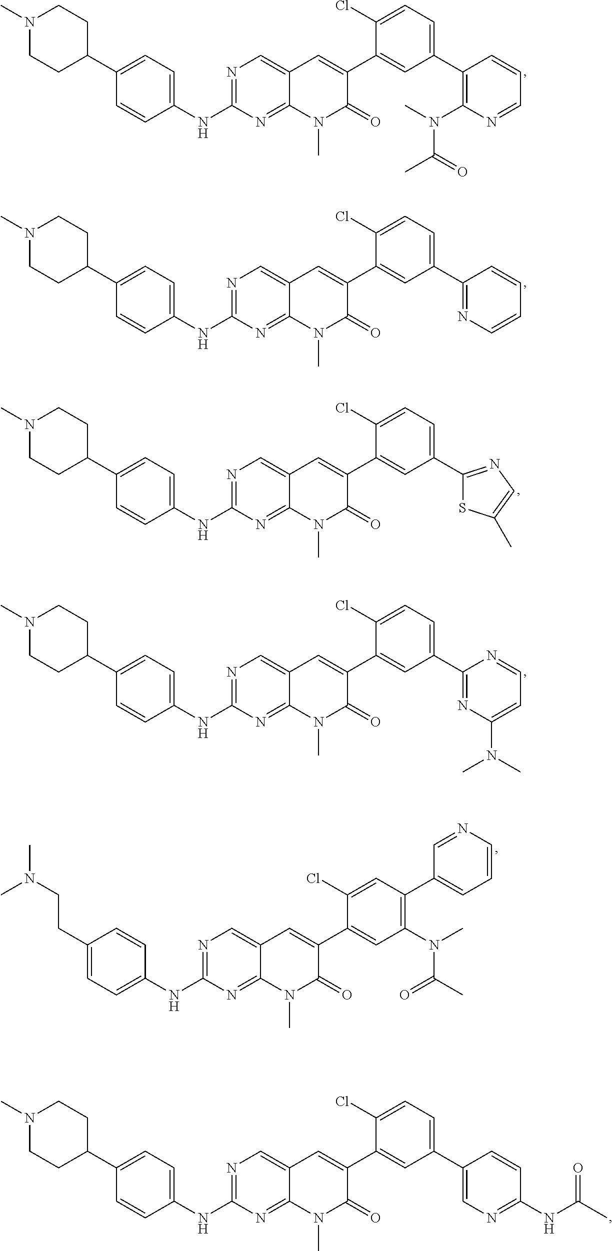

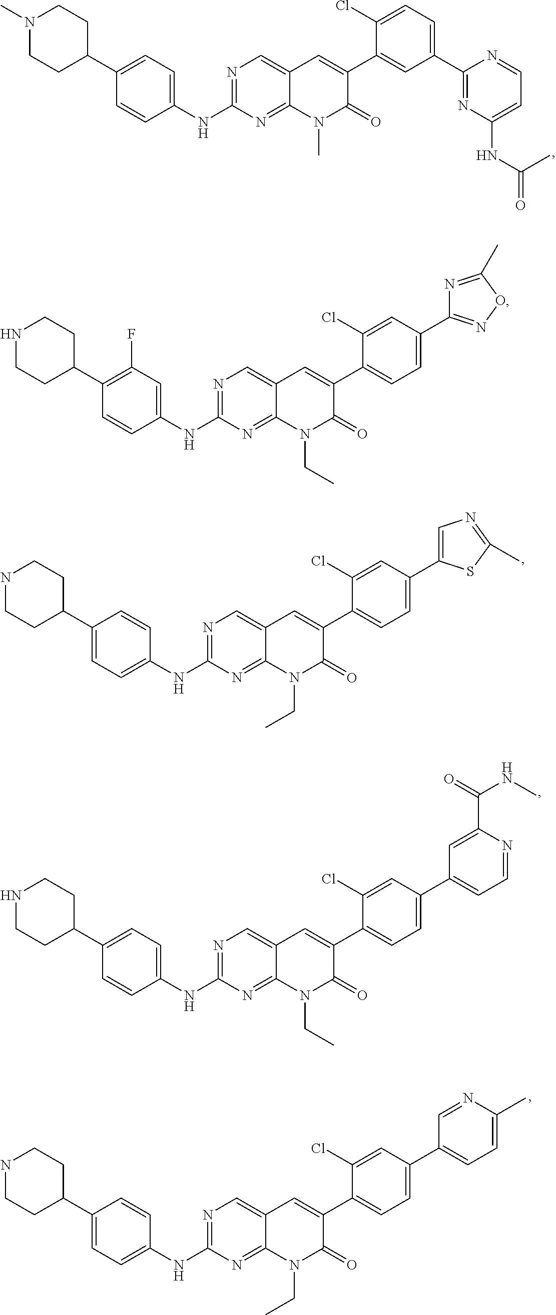

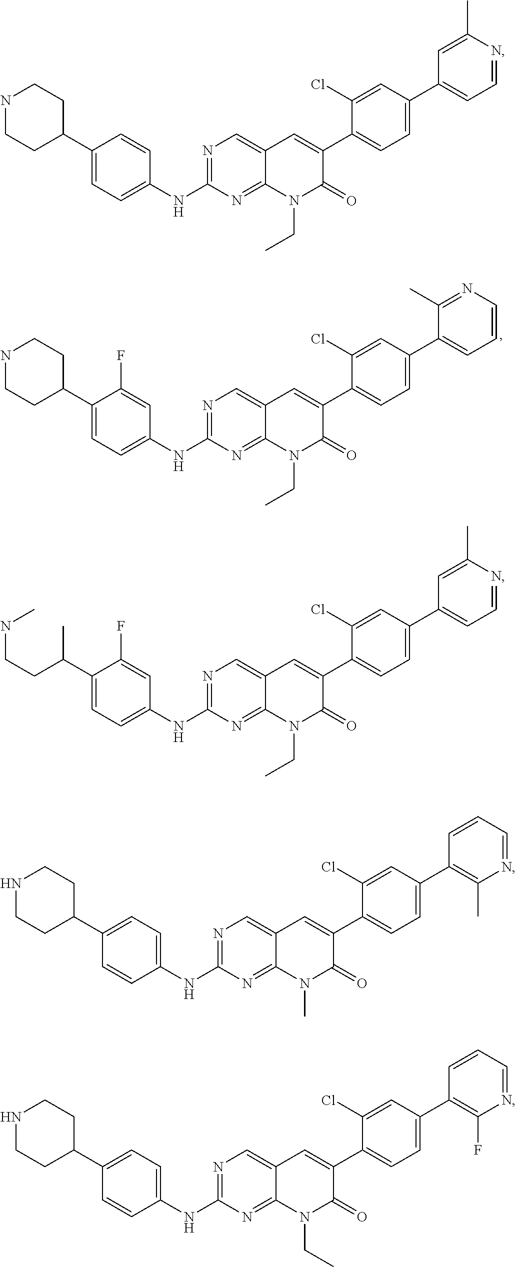 [SMILES: CCn1c(=O)c(-c2ccc(-c3ccnc(C)c3)cc2Cl)cc2cnc(Nc3ccc(C4CCNCC4)cc3)nc21 ; CCn1c(=O)c(-c2ccc(-c3cccnc3C)cc2Cl)cc2cnc(Nc3ccc(C4CCNCC4)c(F)c3)nc21 ; CCn1c(=O)c(-c2ccc(-c3ccnc(C)c3)cc2Cl)cc2cnc(Nc3ccc(C(C)CCNC)c(F)c3)nc21 ; Cc1ncccc1-c1ccc(-c2cc3cnc(Nc4ccc(C5CCNCC5)cc4)nc3n(C)c2=O)c(Cl)c1 ; CCn1c(=O)c(-c2ccc(-c3cccnc3F)cc2Cl)cc2cnc(Nc3ccc(C4CCNCC4)cc3)nc21]

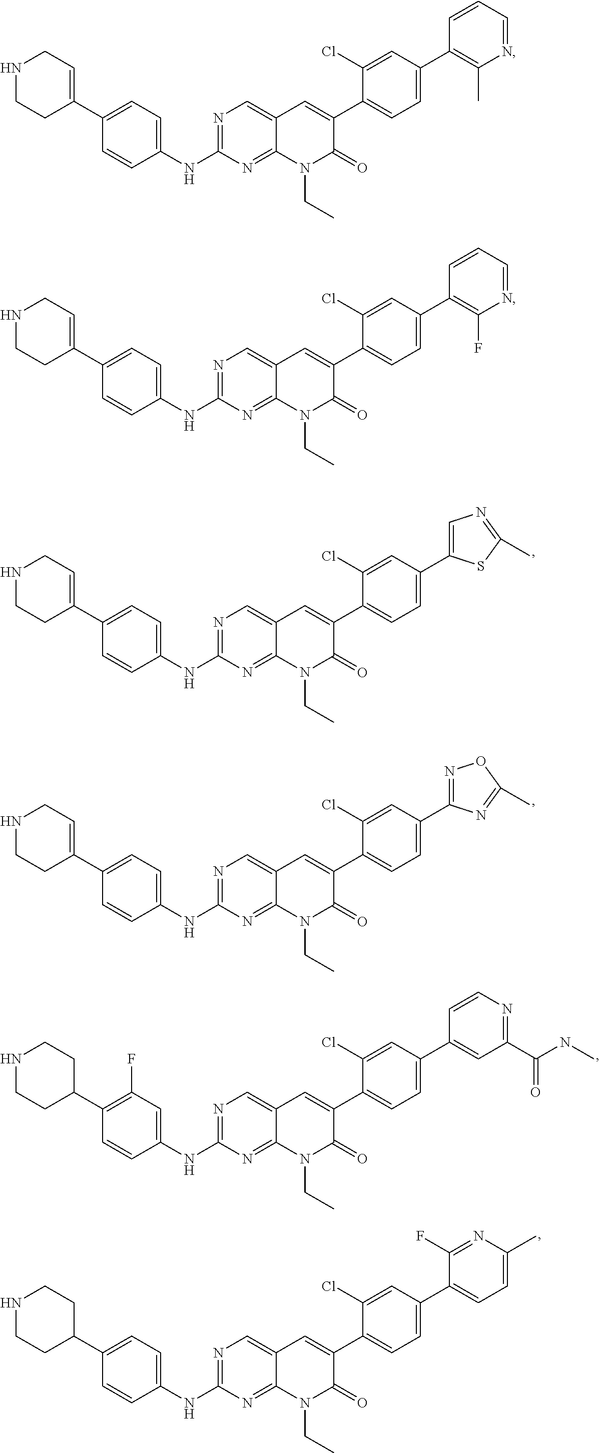 [SMILES: CCn1c(=O)c(-c2ccc(-c3cccnc3C)cc2Cl)cc2cnc(Nc3ccc(C4=CCNCC4)cc3)nc21 ; CCn1c(=O)c(-c2ccc(-c3cccnc3F)cc2Cl)cc2cnc(Nc3ccc(C4=CCNCC4)cc3)nc21 ; CCn1c(=O)c(-c2ccc(-c3cnc(C)s3)cc2Cl)cc2cnc(Nc3ccc(C4=CCNCC4)cc3)nc21 ; CCn1c(=O)c(-c2ccc(-c3noc(C)n3)cc2Cl)cc2cnc(Nc3ccc(C4=CCNCC4)cc3)nc21 ; CCn1c(=O)c(-c2ccc(-c3ccnc(C(=O)NC)c3)cc2Cl)cc2cnc(Nc3ccc(C4CCNCC4)c(F)c3)nc21 ; CCn1c(=O)c(-c2ccc(-c3ccc(C)nc3F)cc2Cl)cc2cnc(Nc3ccc(C4CCNCC4)cc3)nc21]

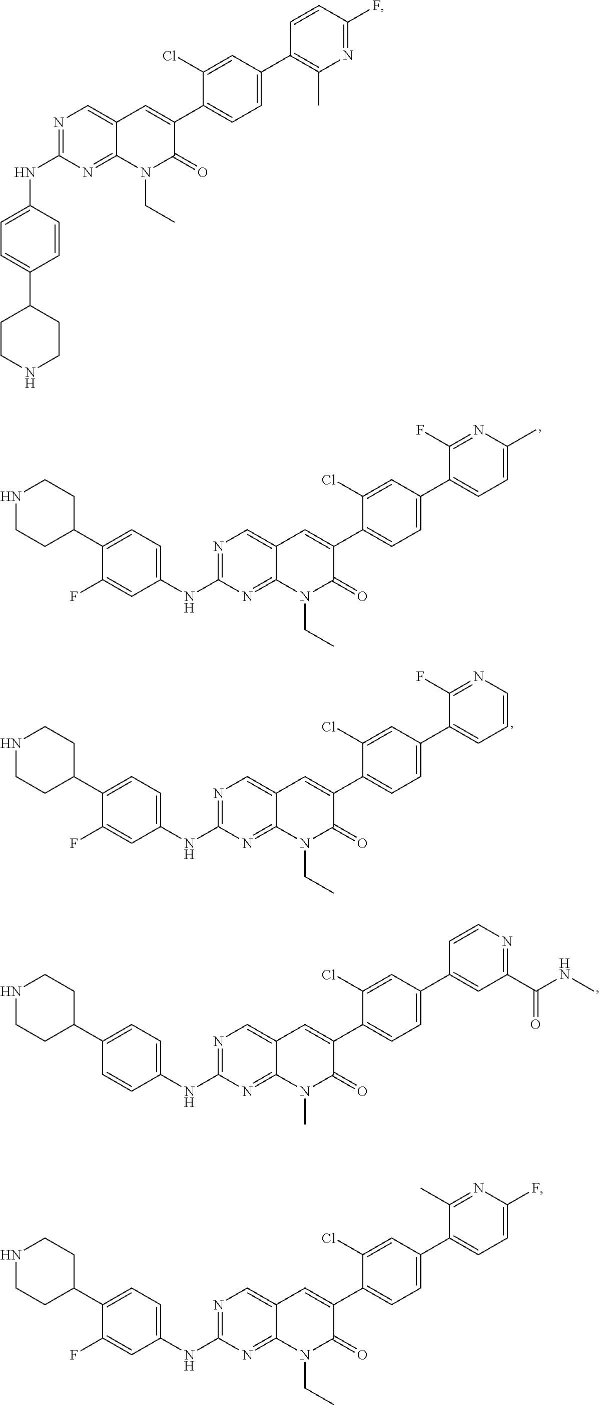 [SMILES: CCn1c(=O)c(-c2ccc(-c3ccc(F)nc3C)cc2Cl)cc2cnc(Nc3ccc(C4CCNCC4)cc3)nc21 ; CCn1c(=O)c(-c2ccc(-c3ccc(C)nc3F)cc2Cl)cc2cnc(Nc3ccc(C4CCNCC4)c(F)c3)nc21 ; CCn1c(=O)c(-c2ccc(-c3cccnc3F)cc2Cl)cc2cnc(Nc3ccc(C4CCNCC4)c(F)c3)nc21 ; CNC(=O)c1cc(-c2ccc(-c3cc4cnc(Nc5ccc(C6CCNCC6)cc5)nc4n(C)c3=O)c(Cl)c2)ccn1 ; CCn1c(=O)c(-c2ccc(-c3ccc(F)nc3C)cc2Cl)cc2cnc(Nc3ccc(C4CCNCC4)c(F)c3)nc21]

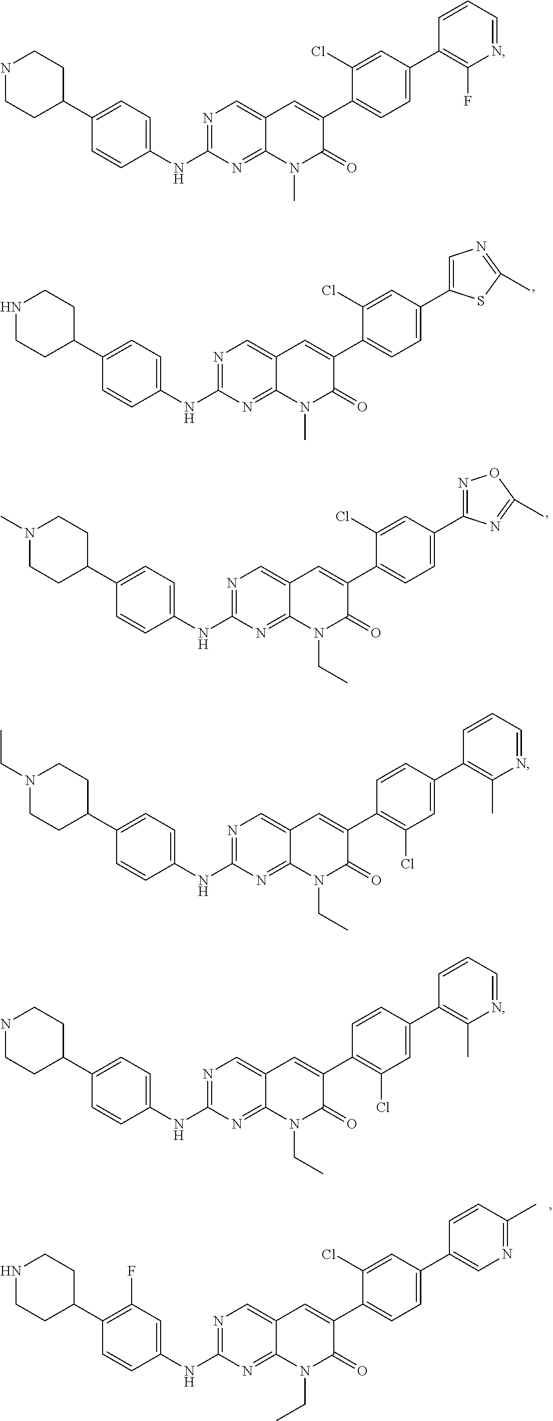

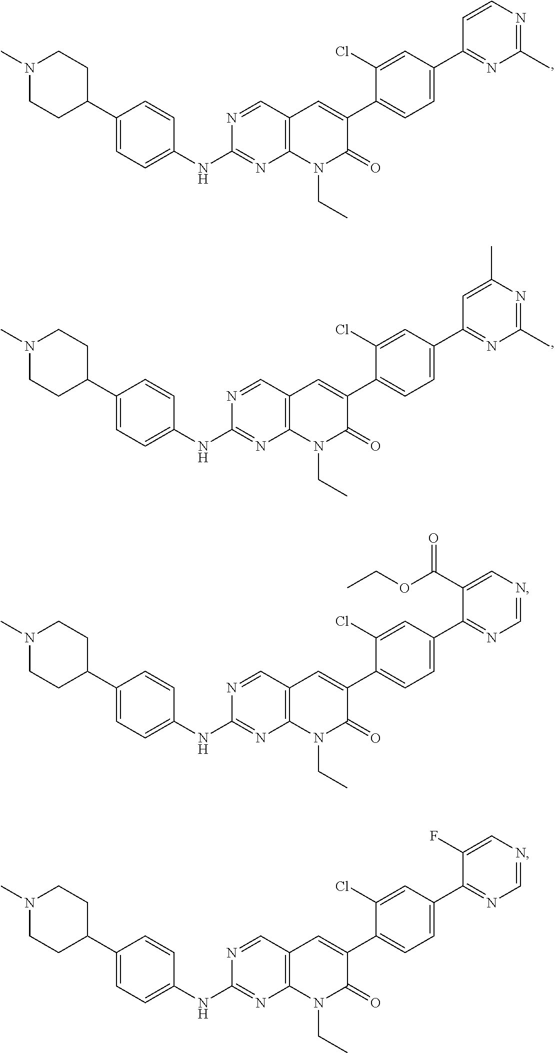

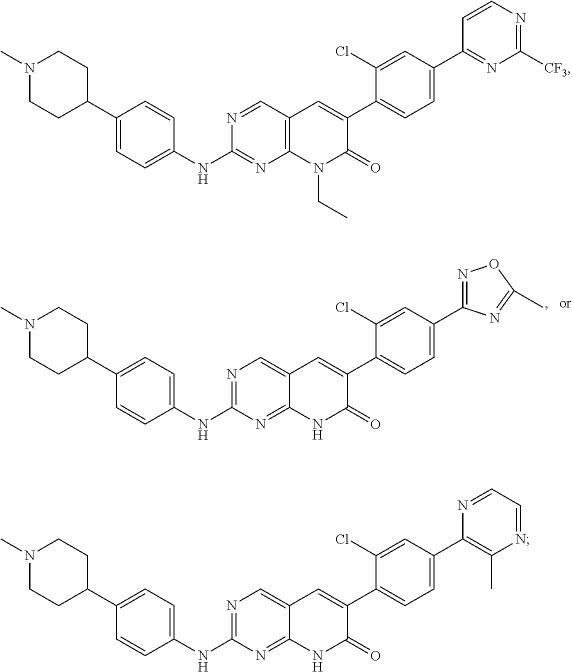

or a pharmaceutically acceptable salt, solvate or N-oxide thereof.

In another embodiment are PAK inhibitors suitable for treating cancer having the structure:

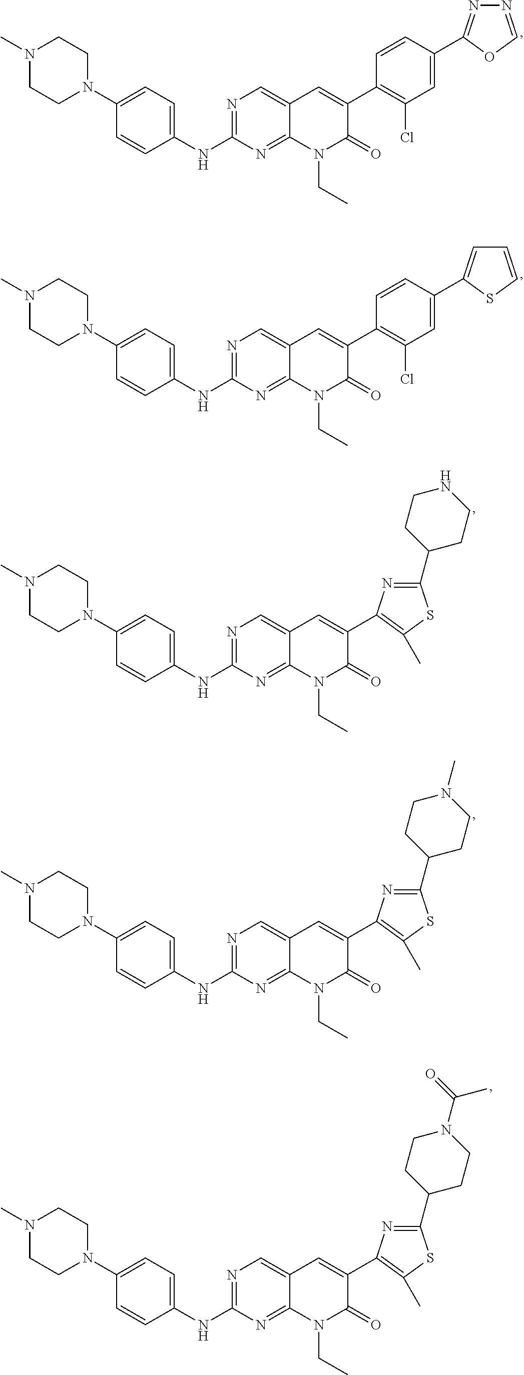 [SMILES: CCn1c(=O)c(-c2ccc(-c3nnco3)cc2Cl)cc2cnc(Nc3ccc(N4CCN(C)CC4)cc3)nc21 ; CCn1c(=O)c(-c2ccc(-c3cccs3)cc2Cl)cc2cnc(Nc3ccc(N4CCN(C)CC4)cc3)nc21 ; CCn1c(=O)c(-c2nc(C3CCNCC3)sc2C)cc2cnc(Nc3ccc(N4CCN(C)CC4)cc3)nc21 ; CCn1c(=O)c(-c2nc(C3CCN(C)CC3)sc2C)cc2cnc(Nc3ccc(N4CCN(C)CC4)cc3)nc21 ; CCn1c(=O)c(-c2nc(C3CCN(C(C)=O)CC3)sc2C)cc2cnc(Nc3ccc(N4CCN(C)CC4)cc3)nc21]

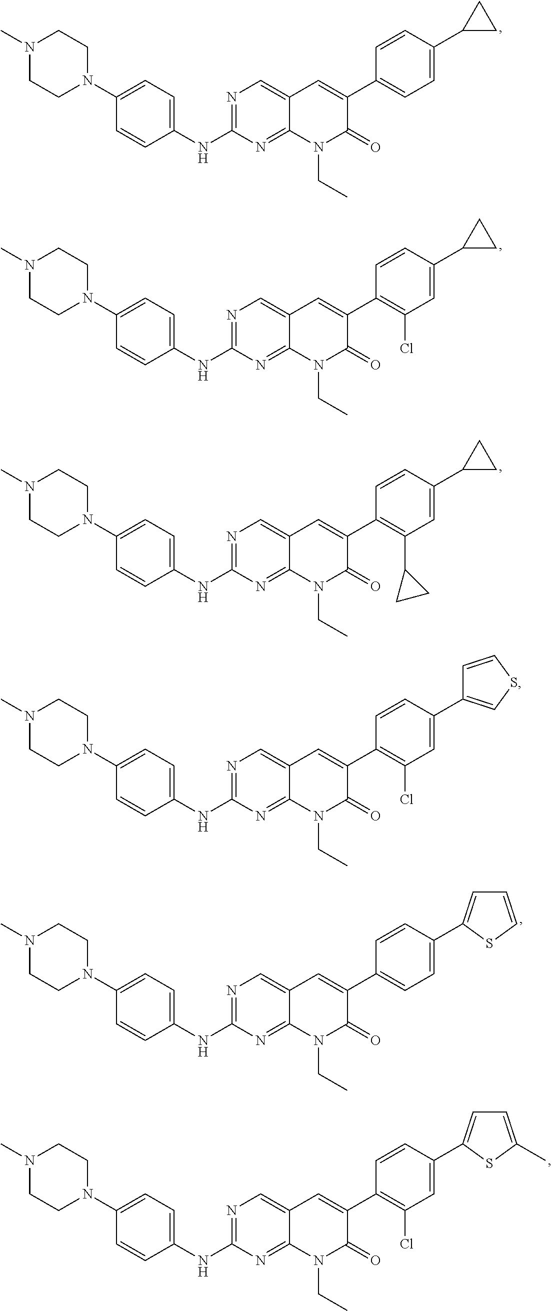

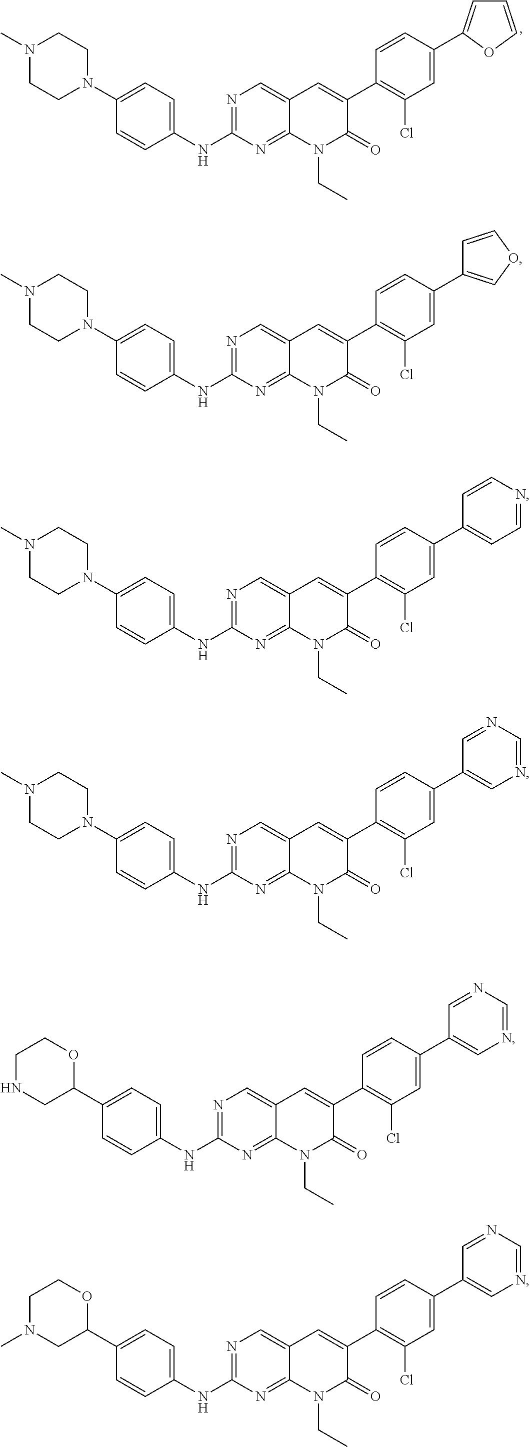 [SMILES: CCn1c(=O)c(-c2ccc(-c3ccco3)cc2Cl)cc2cnc(Nc3ccc(N4CCN(C)CC4)cc3)nc21 ; CCn1c(=O)c(-c2ccc(-c3ccoc3)cc2Cl)cc2cnc(Nc3ccc(N4CCN(C)CC4)cc3)nc21 ; CCn1c(=O)c(-c2ccc(-c3ccncc3)cc2Cl)cc2cnc(Nc3ccc(N4CCN(C)CC4)cc3)nc21 ; CCn1c(=O)c(-c2ccc(-c3cncnc3)cc2Cl)cc2cnc(Nc3ccc(N4CCN(C)CC4)cc3)nc21 ; CCn1c(=O)c(-c2ccc(-c3cncnc3)cc2Cl)cc2cnc(Nc3ccc(C4CNCCO4)cc3)nc21 ; CCn1c(=O)c(-c2ccc(-c3cncnc3)cc2Cl)cc2cnc(Nc3ccc(C4CN(C)CCO4)cc3)nc21]

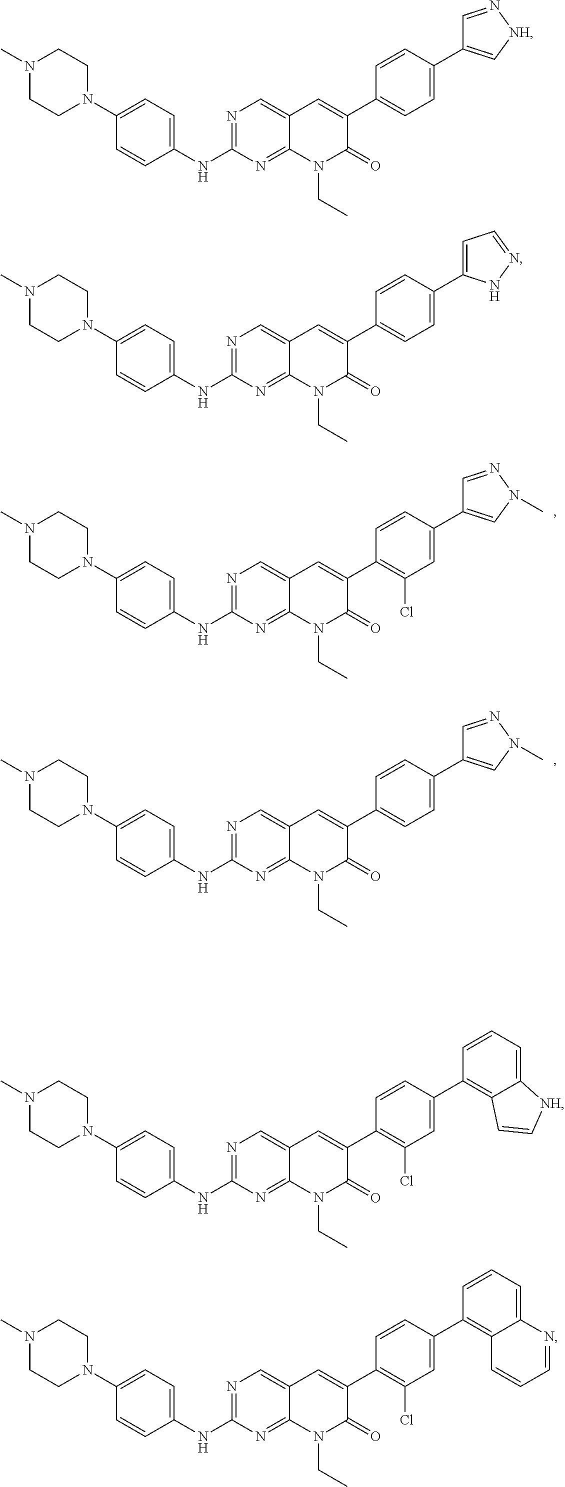

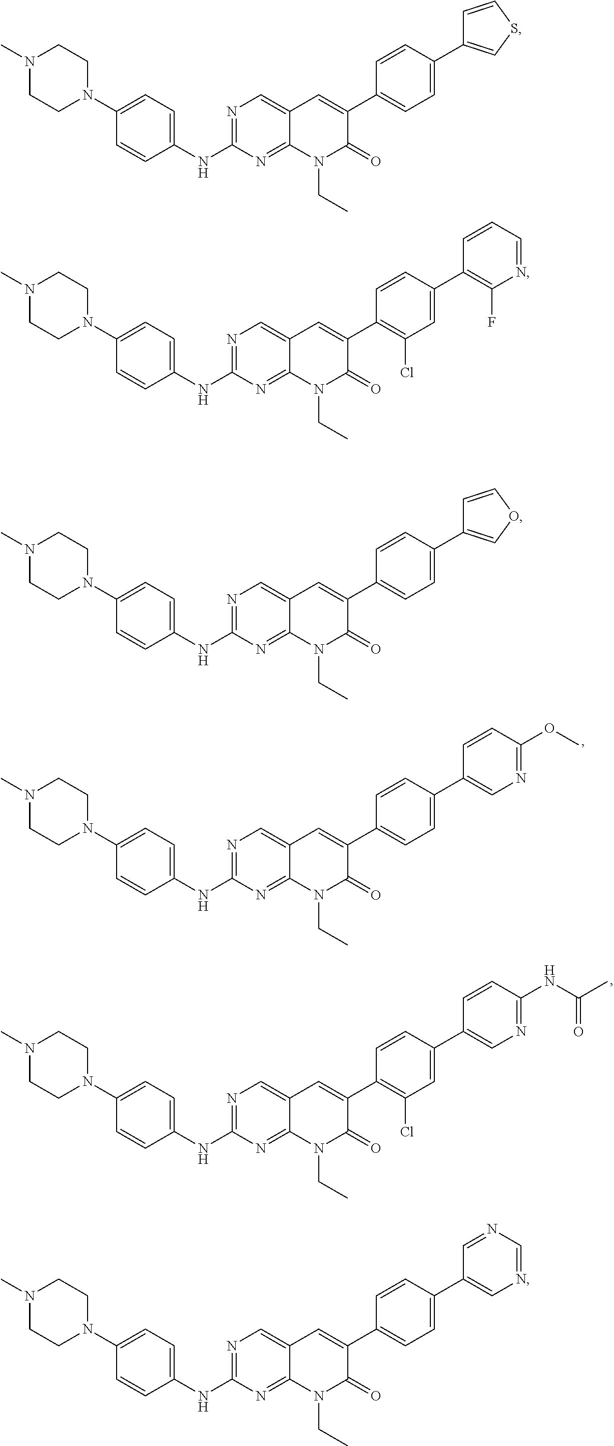

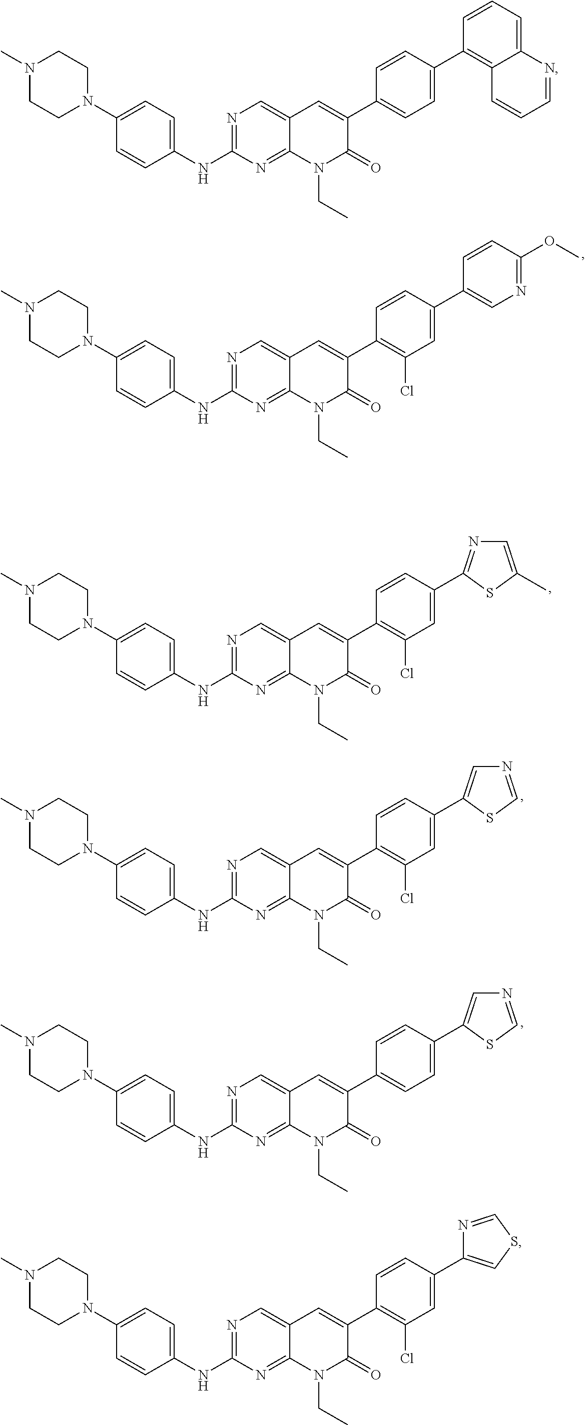

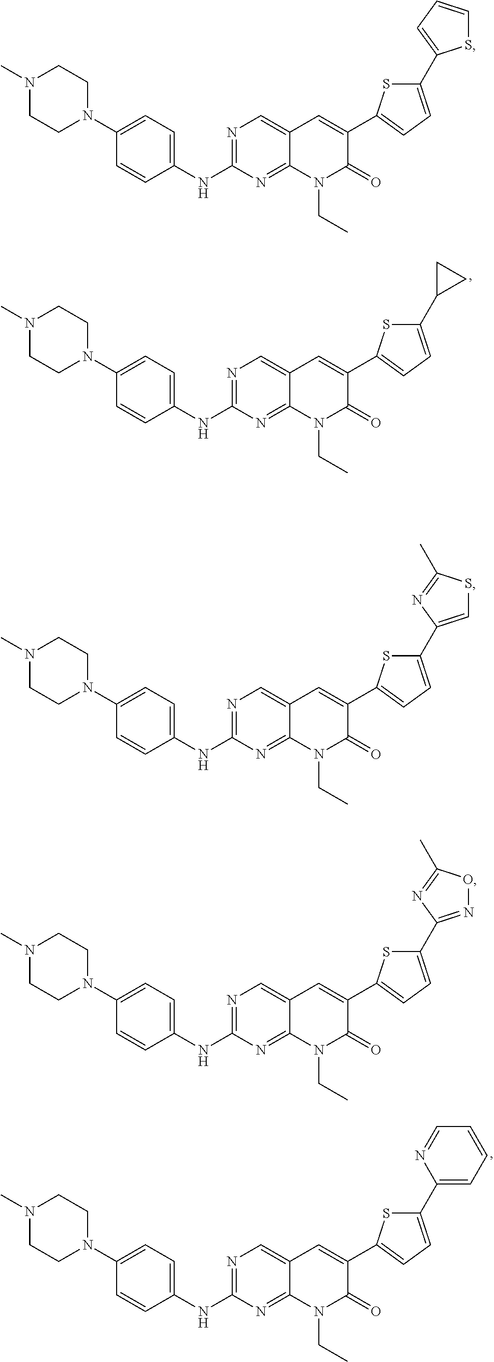 [SMILES: CCn1c(=O)c(-c2ccc(-c3cccs3)s2)cc2cnc(Nc3ccc(N4CCN(C)CC4)cc3)nc21 ; CCn1c(=O)c(-c2ccc(C3CC3)s2)cc2cnc(Nc3ccc(N4CCN(C)CC4)cc3)nc21 ; CCn1c(=O)c(-c2ccc(-c3csc(C)n3)s2)cc2cnc(Nc3ccc(N4CCN(C)CC4)cc3)nc21 ; CCn1c(=O)c(-c2ccc(-c3noc(C)n3)s2)cc2cnc(Nc3ccc(N4CCN(C)CC4)cc3)nc21 ; CCn1c(=O)c(-c2ccc(-c3ccccn3)s2)cc2cnc(Nc3ccc(N4CCN(C)CC4)cc3)nc21]

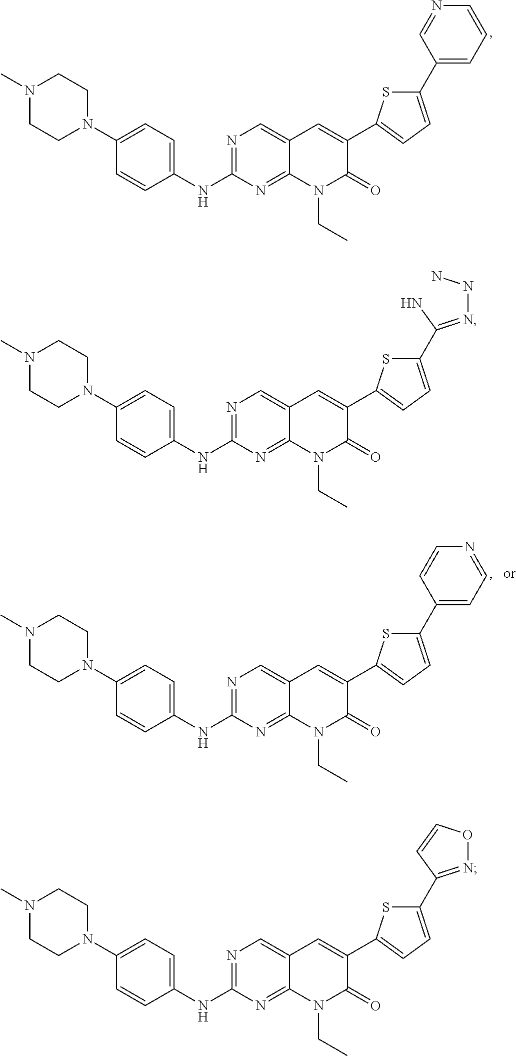 [SMILES: CCn1c(=O)c(-c2ccc(-c3cccnc3)s2)cc2cnc(Nc3ccc(N4CCN(C)CC4)cc3)nc21 ; CCn1c(=O)c(-c2ccc(/C(N)=N/NN)s2)cc2cnc(Nc3ccc(N4CCN(C)CC4)cc3)nc21 ; CCn1c(=O)c(-c2ccc(-c3ccncc3)s2)cc2cnc(Nc3ccc(N4CCN(C)CC4)cc3)nc21 ; CCn1c(=O)c(-c2ccc(-c3ccon3)s2)cc2cnc(Nc3ccc(N4CCN(C)CC4)cc3)nc21]

or a pharmaceutically acceptable salt, solvate or N-oxide thereof.

In another embodiment are PAK inhibitors suitable for treating cancer having the structure of Formula III or a pharmaceutically acceptable salt, solvate or N-oxide thereof wherein:

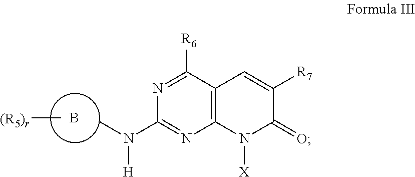

wherein:

### X is

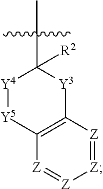 [0338] each of Y³, Y⁴ and Y⁵ is independently a bond, O, N, N—R¹, C—R¹, C(R¹)₂, S, SO₂, or C═O; provided that Y³ is not a bond and at least one Y³, Y⁴ or Y⁵ is selected from O, N—R¹, S, or SO₂;
each Z is independently N or C—R⁴;
each R¹ is independently selected from hydrogen, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or two R¹ together with the atom to which they are attached form a ring;
R² is H or a substituted or unsubstituted alkyl;
each R⁴ is independently hydrogen, halogen, —CN, —NO₂, —OH, —OCF₃, —OCF₃, —OCF₂H, —CF₃, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl;
ring B is aryl or heteroaryl;
each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, substituted or unsubstituted alkyl, substituted or unsubstituted alkoxy, substituted or unsubstituted heteroalkyl, substituted or unsubstituted cycloalkyl or substituted or unsubstituted heterocycloalkyl;
r is 0-8;
R⁷ is H, halogen, —CN, substituted or unsubstituted alkyl, —C(═O)N(R¹⁰)₂, —CO₂R¹⁰, —OR¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
R⁶ is H, halogen, —OR, —NRR, a substituted or unsubstituted alkyl, a substituted or unsubstituted cycloalkyl, a substituted or unsubstituted heterocycloalkyl, a substituted or unsubstituted aryl, a substituted or unsubstituted heteroaryl;
R⁸ is H or R⁹;
R⁹ is a substituted or unsubstituted alkyl, a substituted or unsubstituted cycloalkyl, a substituted or unsubstituted heterocycloalkyl, a substituted or unsubstituted aryl, or a substituted or unsubstituted heteroarylyl; and
each R¹⁰ is independently H, a substituted or unsubstituted alkyl, a substituted or unsubstituted cycloalkyl, a substituted or unsubstituted heterocycloalkyl, a substituted or unsubstituted aryl, or a substituted or unsubstituted heteroaryl; or two R¹⁰, together with the atoms to which they are attached, form a heterocycle.

In one embodiment is a compound of Formula III wherein X is selected from:

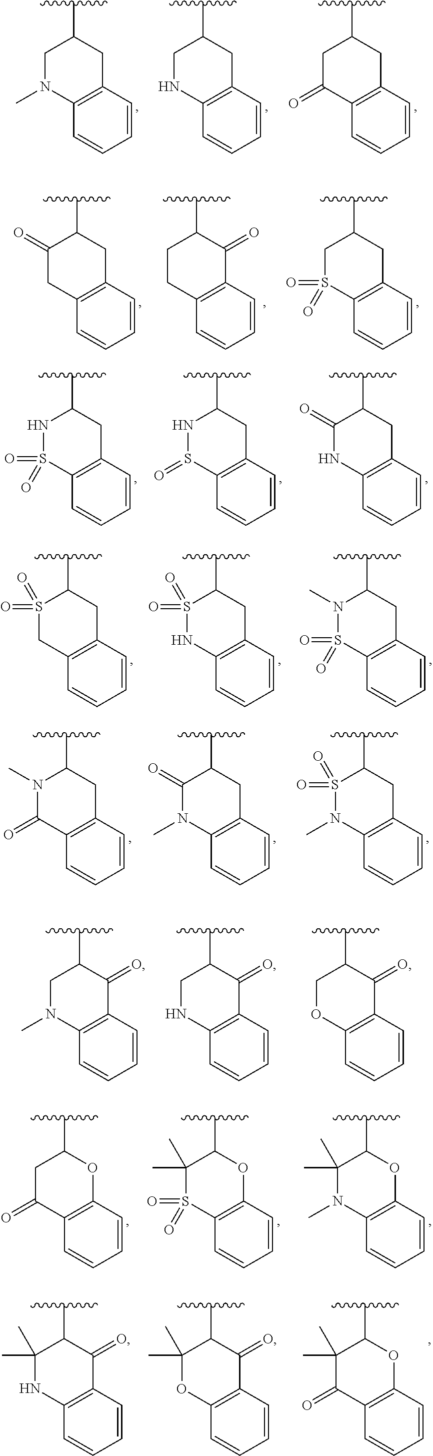

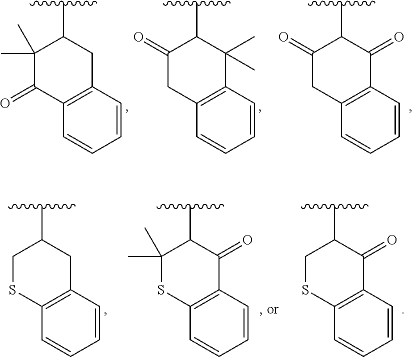

In another embodiment, the phenyl ring of X is substituted with at least one substituent selected from halogen, —CN, —NO₂, —OH, —OCF₃, —OCF₃, —OCF₂H, —CF₃, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, a substituted or unsubstituted alkyl, a substituted or unsubstituted alkoxy, a substituted or unsubstituted heteroalkyl, a substituted or unsubstituted cycloalkyl, or a substituted or unsubstituted heterocycloalkyl. In one embodiment the phenyl ring of X is substituted with at least one halogen. In another embodiment, at least two halogens. In a further embodiment, the phenyl ring of X is substituted with at least one —OH. In another embodiment, at least one CF₃.

In yet another embodiment, is a compound of Formula III, wherein ring B is an aryl ring. In another embodiment, ring B is a substituted or unsubstituted phenyl. In a further embodiment, ring B is a substituted or unsubstituted naphthalene. In a further embodiment, is a compound of Formula III, wherein ring B is a heteroaryl ring selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, and pyrazine.

In another embodiment is a compound of Formula III wherein ring B is

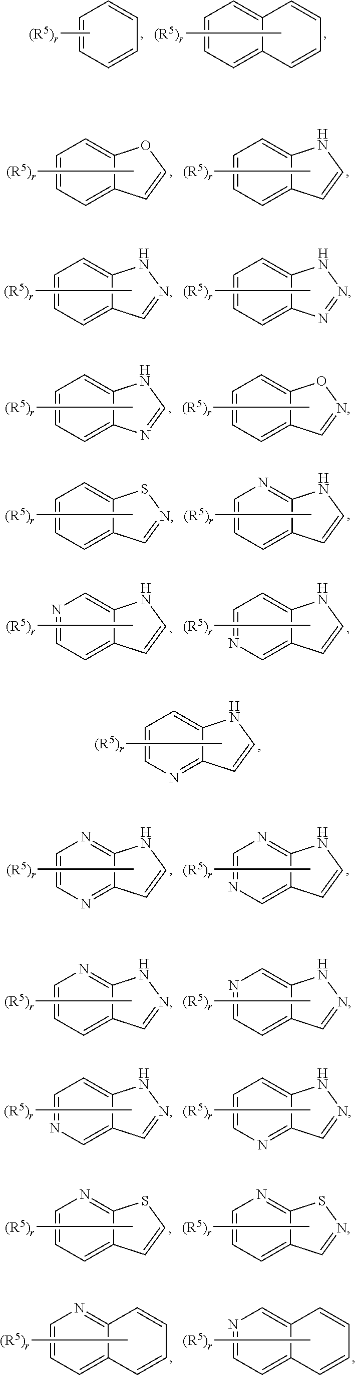

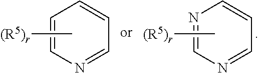

In one embodiment, is a compound of Formula III, wherein R⁵ is halogen, —CN, —OH, substituted or unsubstituted alkyl, —OR¹⁰, —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In one embodiment, R⁵ is selected from F, Cl, Br, or I. In another embodiment R⁵ is F.

In another embodiment, is a compound of Formula III, wherein at least one R⁵ is —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In one embodiment, is a compound of Formula III, wherein at least one R⁵ is —N(R¹⁰)₂, or substituted or unsubstituted heterocycloalkyl. In yet another embodiment, is a compound of Formula III wherein at least one of R⁵ is a substituted or unsubstituted piperazine, substituted or unsubstituted piperidine, substituted or unsubstituted pyrrolidine or substituted or unsubstituted morpholine. In a further embodiment, is a compound of Formula III, wherein at least one R⁵ is —OR¹⁰. In one embodiment is a compound of Formula III, wherein at least one R⁵ is —OR¹⁰ and R¹⁰ is H. In another embodiment, R¹⁰ is alkyl selected from methyl, ethyl, n-propyl, iso-propyl, n-butyl, iso-butyl, and tert-butyl.

In another embodiment, is a compound of Formula III wherein R⁵ is a C₃-C₆cycloalkyl ring. In another embodiment, R⁵ is cyclopropyl. In another embodiment, R⁵ is cyclopentyl. In another embodiment, R⁵ is cyclohexyl.

In another embodiment, is a compound of Formula III wherein R⁵ is a substituted or unsubstituted heterocycloalkyl ring. In another embodiment, R⁵ is a substituted or unsubstituted piperazine. In another embodiment, R⁵ is an unsubstituted piperazine. In another embodiment, R⁵ is N-methylpiperazine. In another embodiment, R⁵ is a substituted or unsubstituted piperidine. In another embodiment, R⁵ is an unsubstituted piperidine.

In another embodiment is a compound of Formula III wherein r is 0. In another embodiment, r is 1. In a further embodiment, r is 2. In another embodiment is a compound of Formula III wherein r is 1; and R⁵ is substituted or unsubstituted piperazine.

In one embodiment is a compound of Formula III wherein ring B is substituted with —N(R¹⁰)₂, wherein R¹⁰ is each independently selected from H and a substituted or unsubstituted heterocycloalkyl. In another embodiment is a compound of Formula III wherein ring B is substituted with —NHR¹⁰ wherein R¹⁰ is a substituted or unsubstituted piperazine, substituted or unsubstituted piperidine, substituted or unsubstituted pyrrolidine or substituted or unsubstituted morpholine. In a further embodiment is a compound of Formula III wherein ring B is substituted with —N(CH₃)R¹⁰ wherein R¹⁰ is a substituted or unsubstituted piperazine, substituted or unsubstituted piperidine, substituted or unsubstituted pyrrolidine or substituted or unsubstituted morpholine.

Also presented herein is a compound of Formula III wherein ring B is substituted with —OR¹⁰ wherein R¹⁰ is a substituted or unsubstituted heterocycloalkyl. In another embodiment is a compound of Formula III wherein ring B is substituted with —OR¹⁰ wherein R¹⁰ is a substituted or unsubstituted piperazine, substituted or unsubstituted piperidine, substituted or unsubstituted pyrrolidine or substituted or unsubstituted morpholine. In yet another embodiment is a compound of Formula III wherein ring B is substituted with at least one CF₃.

In yet another embodiment, ring B is substituted with at least two R⁵. In another embodiment, ring B is substituted with halogen and a substituted or unsubstituted heterocycloalkyl. In another embodiment, ring B is substituted with at least one F, Cl, Br, or I and a substituted or unsubstituted piperazine, substituted or unsubstituted piperidine, substituted or unsubstituted pyrrolidine, or substituted or unsubstituted morpholine.

In one embodiment is a compound of Formula III wherein

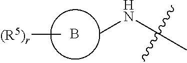

is selected from:

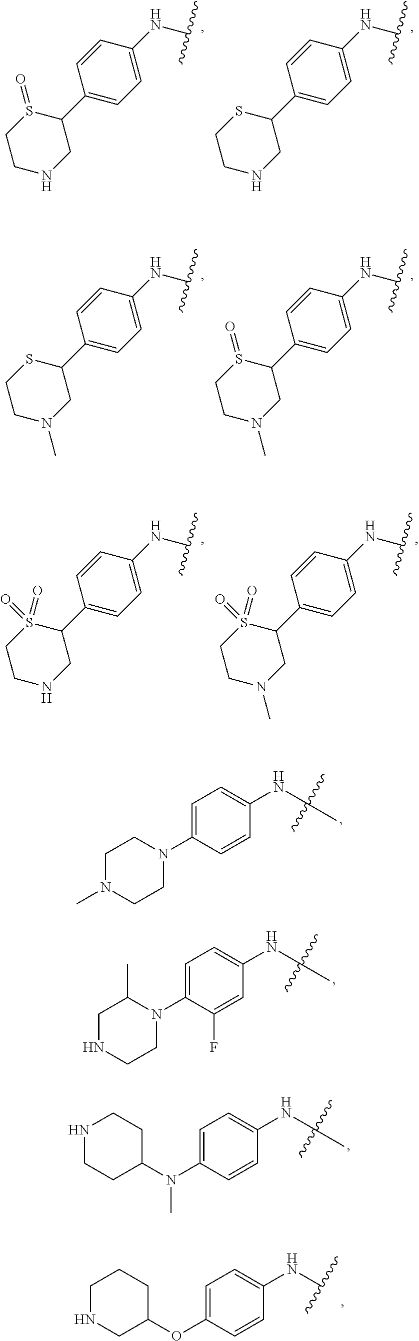

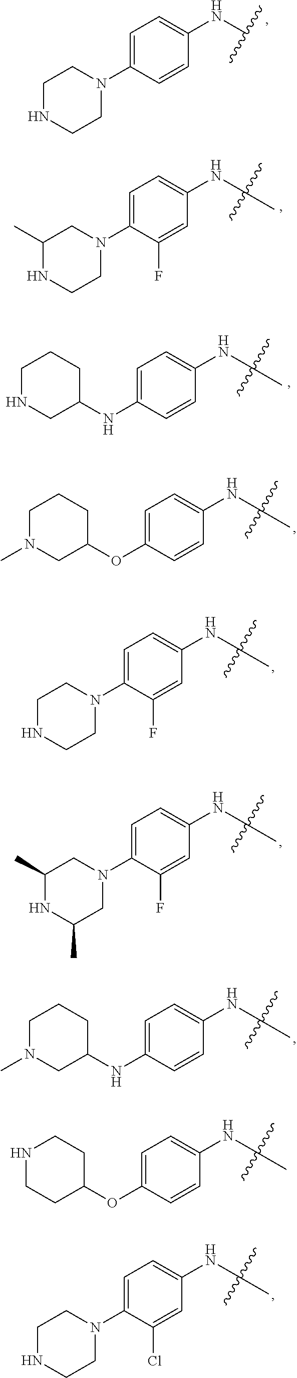

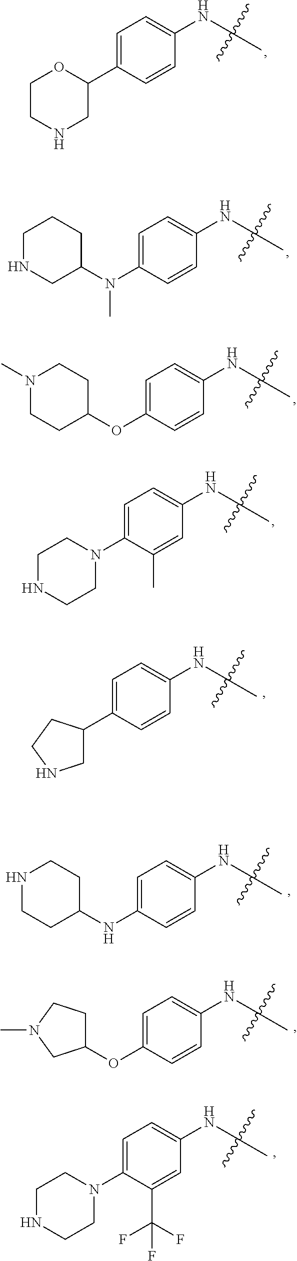

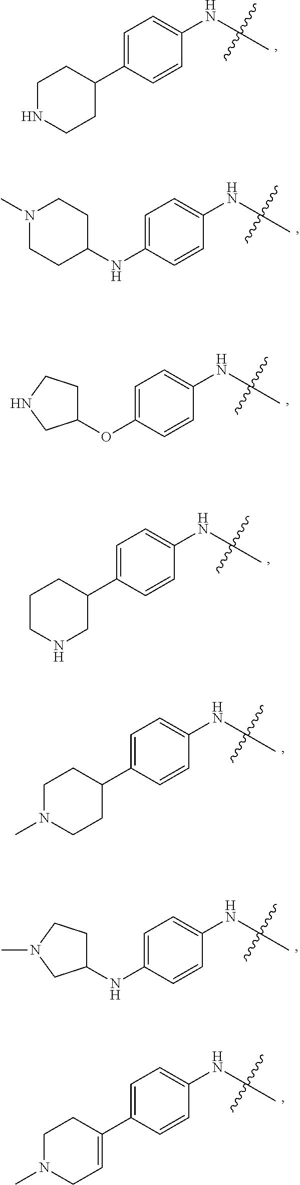

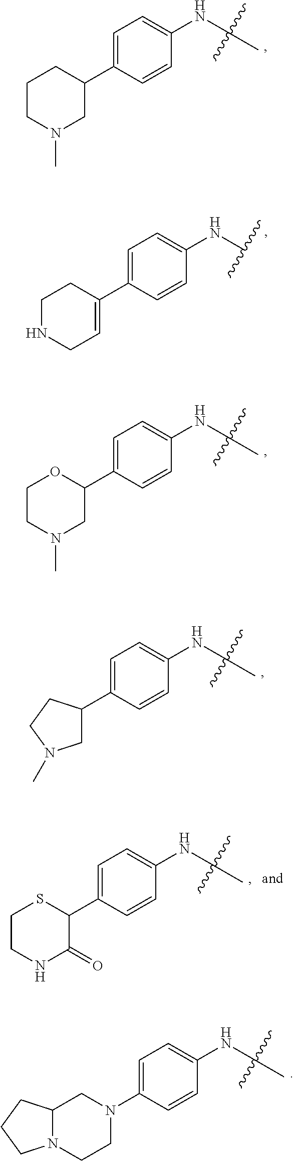 [SMILES: *Nc1ccc(C2CCCN(C)C2)cc1 ; *Nc1ccc(C2=CCNCC2)cc1 ; *Nc1ccc(C2CN(C)CCO2)cc1 ; *Nc1ccc(C2CCN(C)C2)cc1 ; *Nc1ccc(C2SCCNC2=O)cc1 ; *Nc1ccc(N2CCN3CCCC3C2)cc1]

In yet another embodiment is a compound of Formula III wherein

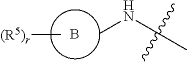

is selected from:

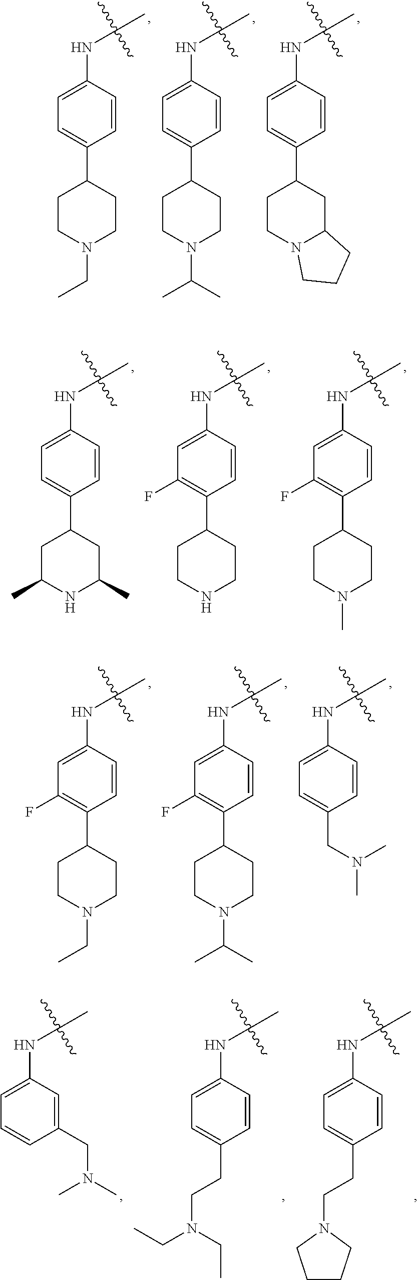

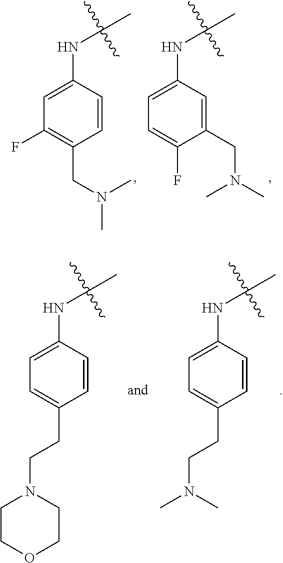

In yet a further embodiment, is a compound of Formula III, wherein R⁴ is independently halogen, —CN, —OH, —OCF₃, —OCHF₂, —OCF₂H, —CF₃, —SR⁸, —S(═O)R⁹, —S(═O)₂R⁹, substituted or unsubstituted alkyl, or substituted or unsubstituted alkoxy. In one embodiment, R⁴ is halogen. In another embodiment, R⁴ is selected from F, Cl, Br, and I. In a further embodiment, R⁴ is F.

In one embodiment, is a compound of Formula III, wherein each Z is CH.

In another embodiment, is a compound of Formula III, wherein R⁷ is H. In yet another embodiment, is a compound of Formula III wherein R⁷ is —CN. In a further embodiment, is a compound of Formula III wherein R⁷ is substituted or unsubstituted cyclopropyl, substituted or unsubstituted cyclobutyl, substituted or unsubstituted cyclopentyl or substituted or unsubstituted cyclohexyl. In another embodiment, R⁷ is —OR¹⁰.

In yet a further embodiment, is a compound of Formula III wherein R⁷ is substituted or unsubstituted morpholino, substituted or unsubstituted piperazinyl, substituted or unsubstituted piperidinyl.

In one embodiment, is a compound of Formula III wherein R⁷ is substituted or unsubstituted acyl.

In another embodiment, is a compound of Formula III, wherein R⁷ is substituted or unsubstituted aryl or substituted or unsubstituted heteroaryl.

In yet another embodiment, is a compound of Formula III wherein R⁷ is

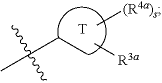

wherein:
ring T is a substituted or unsubstituted aryl or substituted or unsubstituted heteroaryl;
R^{4a} is H or R^{3a};
R^{3a} is halogen, —CN, —NO₂, —OH, —OCF₃, —OCF₂H, —CF₃, —SR⁸, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(O)N(R¹⁰)₂, —OR⁹, substituted or unsubstituted alkyl, substituted or unsubstituted heteroalkyl, substituted or unsubstituted cycloalkyl or substituted or unsubstituted heterocycloalkyl;
R⁸ is H or substituted or unsubstituted alkyl, substituted or unsubstituted cycloalkyl, substituted or unsubstituted heterocycloalkyl, substituted or unsubstituted aryl or substituted or unsubstituted heteroaryl;
R⁹ is substituted or unsubstituted alkyl, substituted or unsubstituted cycloalkyl, substituted or unsubstituted aryl or substituted or unsubstituted heteroaryl, substituted or unsubstituted heterocycloalkyl;
each R¹⁰ is independently H, substituted or unsubstituted alkyl, substituted or unsubstituted cycloalkyl, substituted or unsubstituted aryl or substituted or unsubstituted heteroaryl, substituted or unsubstituted heterocycloalkyl, or two R¹⁰ together with the atoms to which they are attached form a substituted or unsubstituted heterocycle; and
s 0-4.

In a further embodiment, is a compound of Formula III, wherein R³ is cyclopropyl, cyclobutyl, morpholino, piperidinyl, tetrahydropyran, tetrahydrofuranyl, pyrrolidinyl, or piperazinyl.

In yet a further embodiment, is a compound of Formula III, wherein R³ is heteroaryl selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine and pyrazine.

In yet another embodiment is a compound of Formula III wherein R⁷ is selected from:

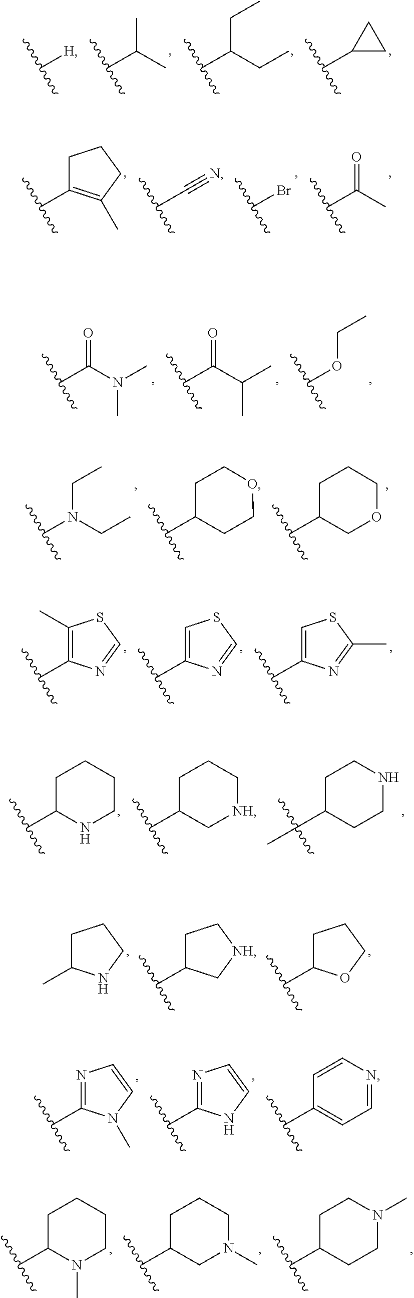

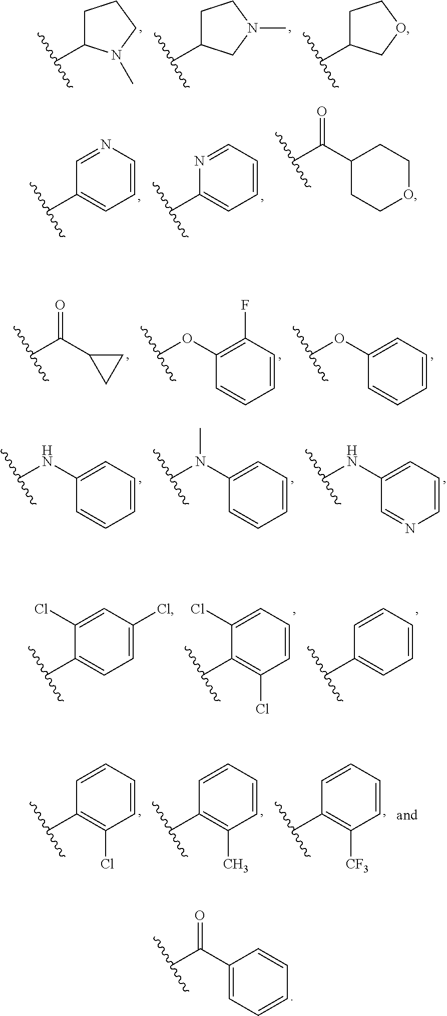

In a further embodiment is a compound having the structure:

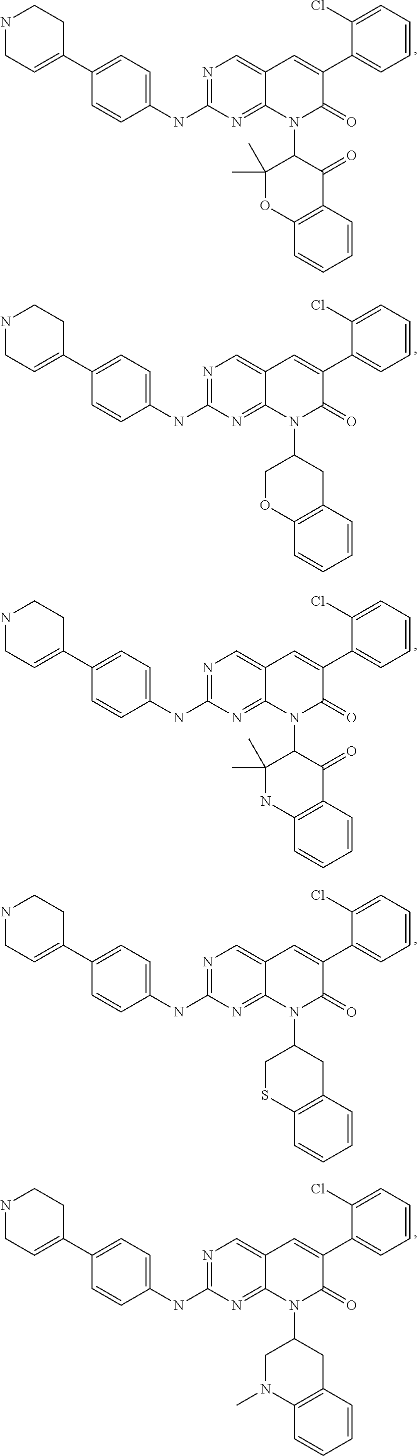

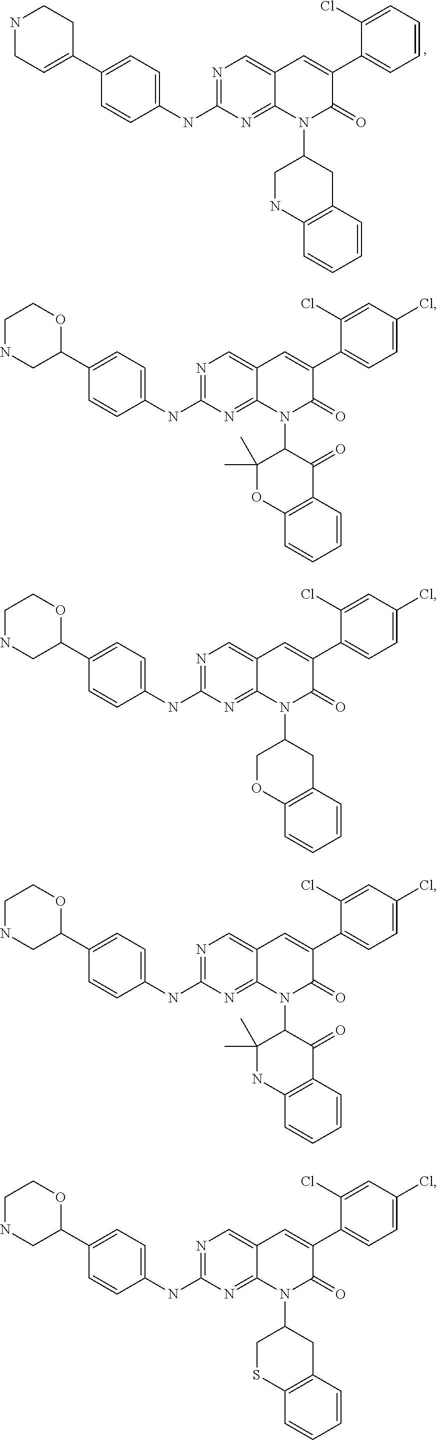

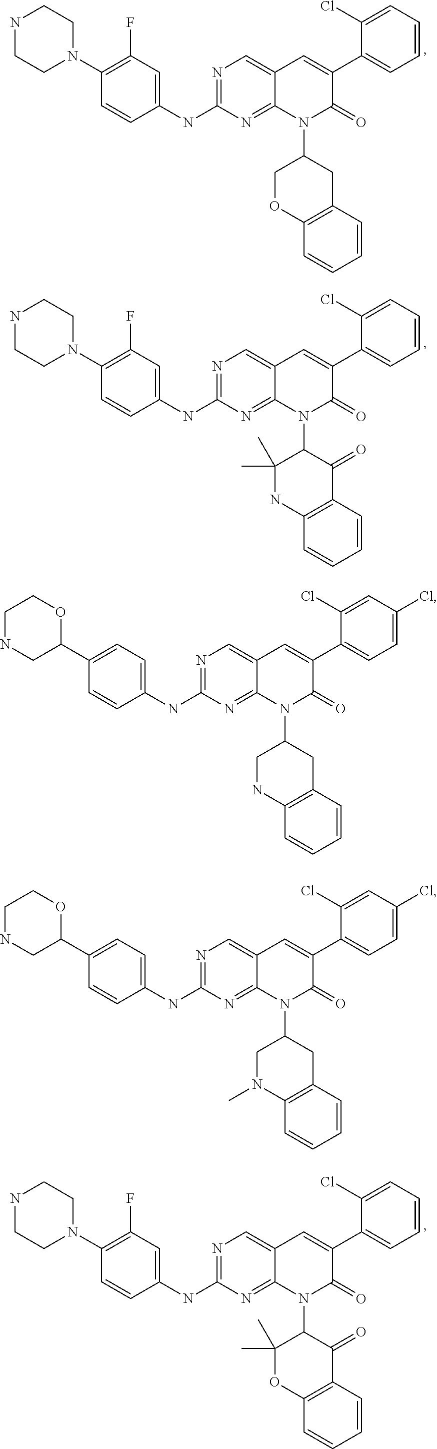

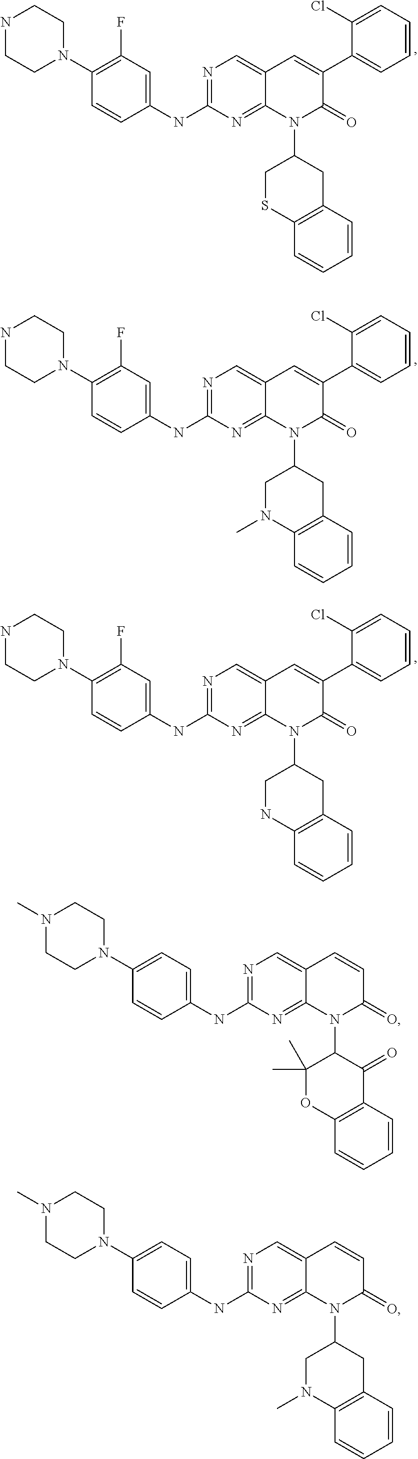

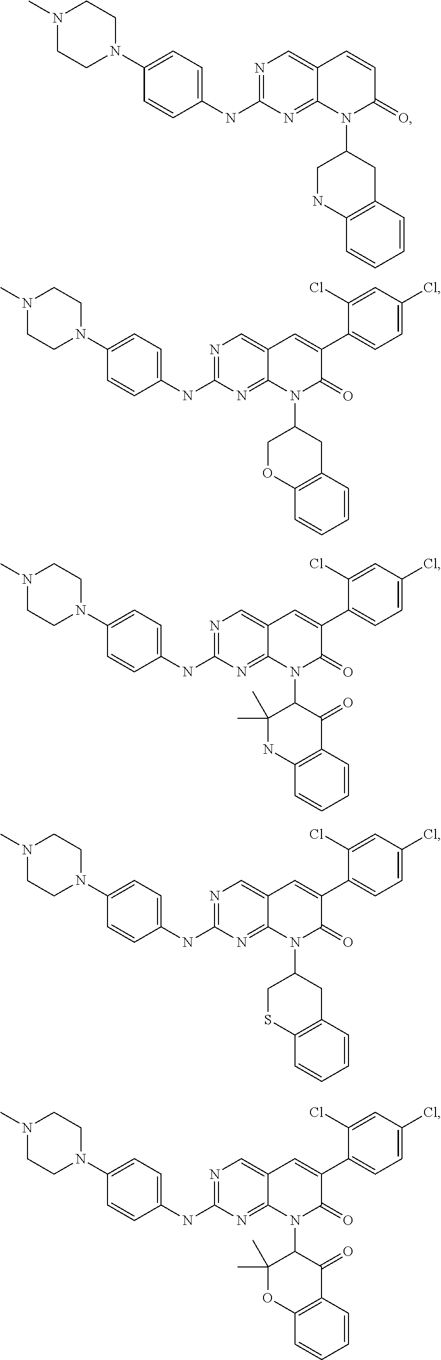

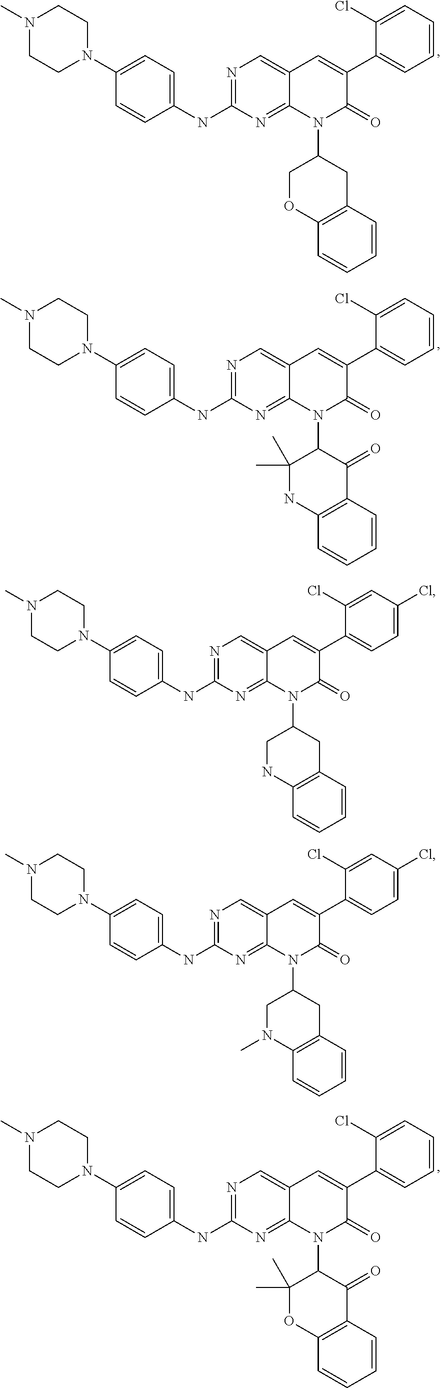

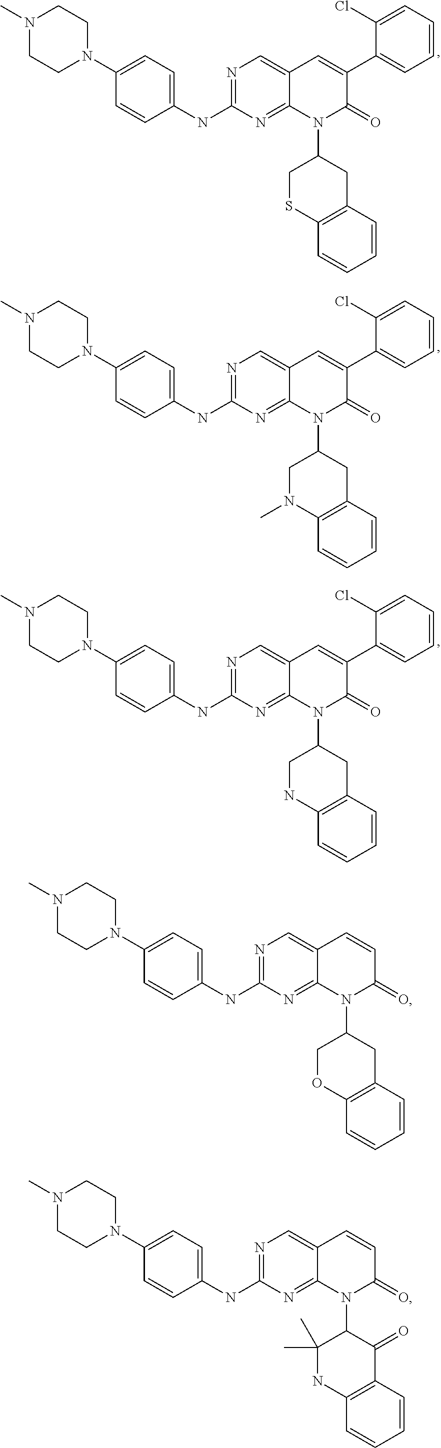

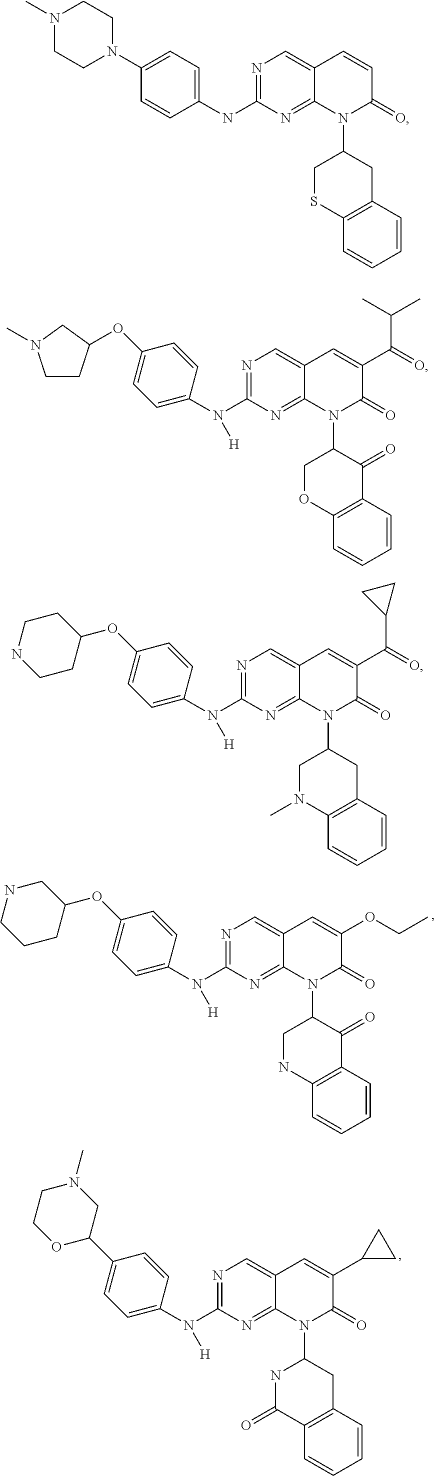

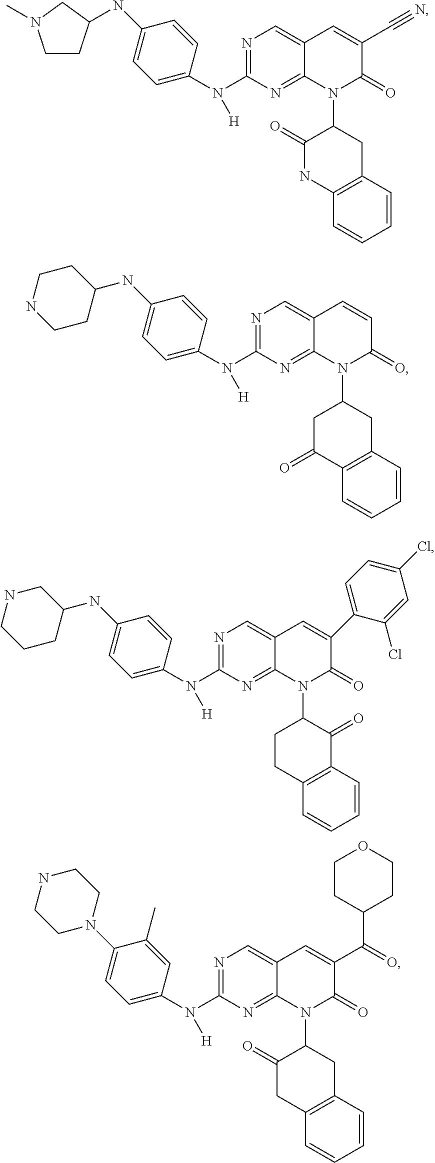

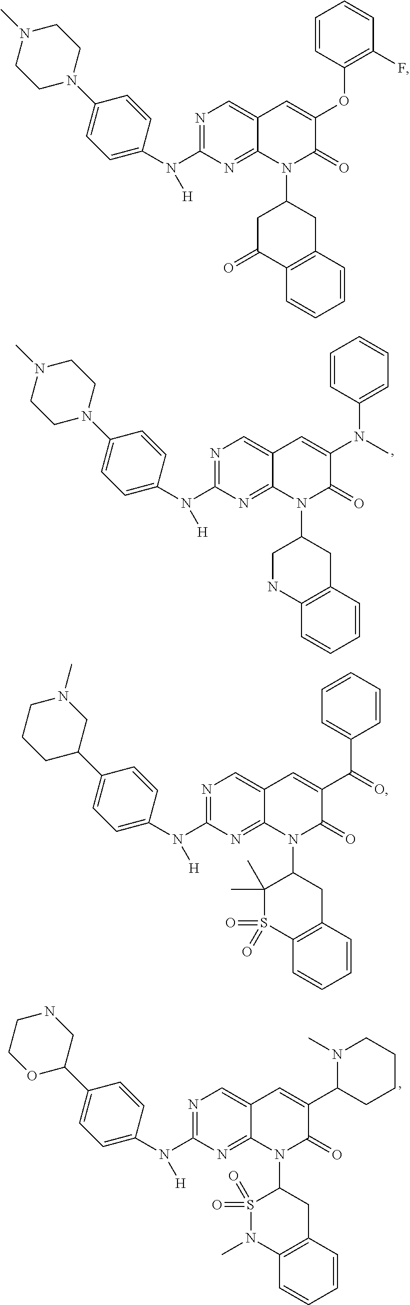

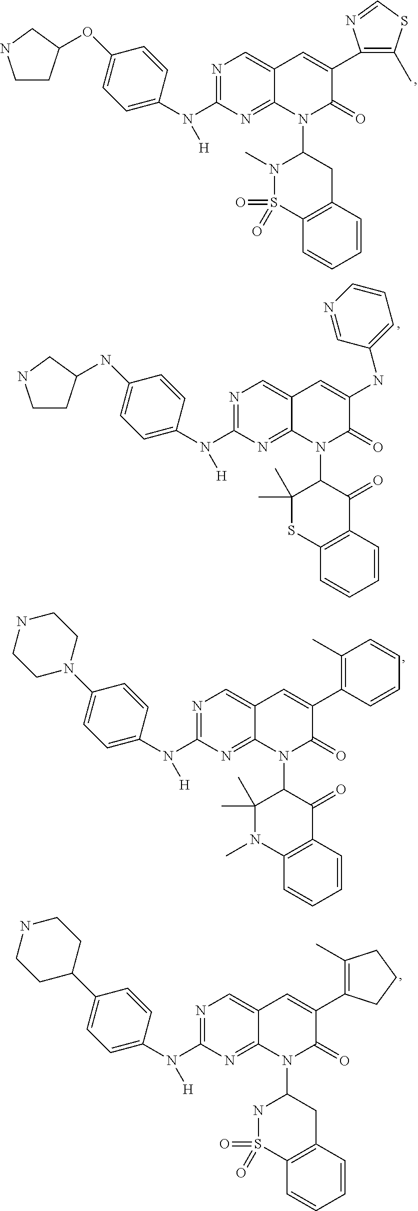

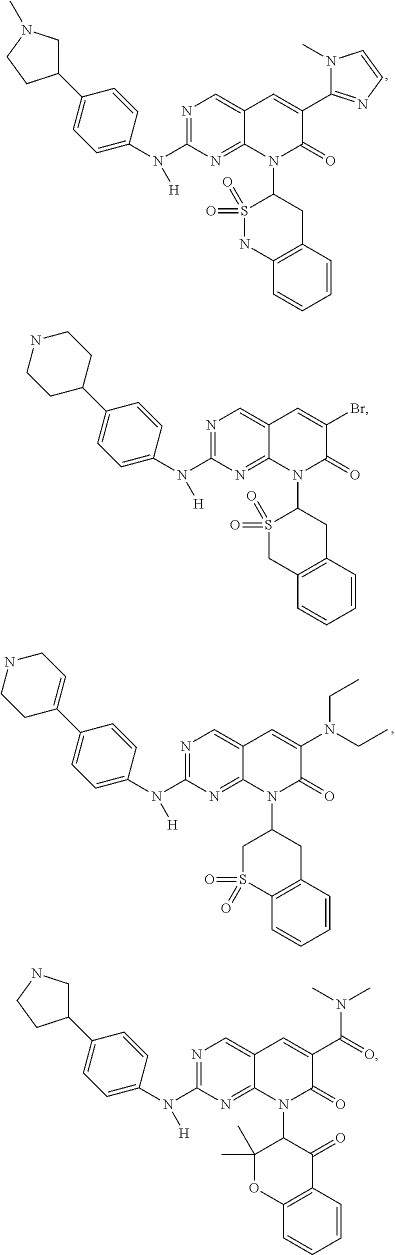

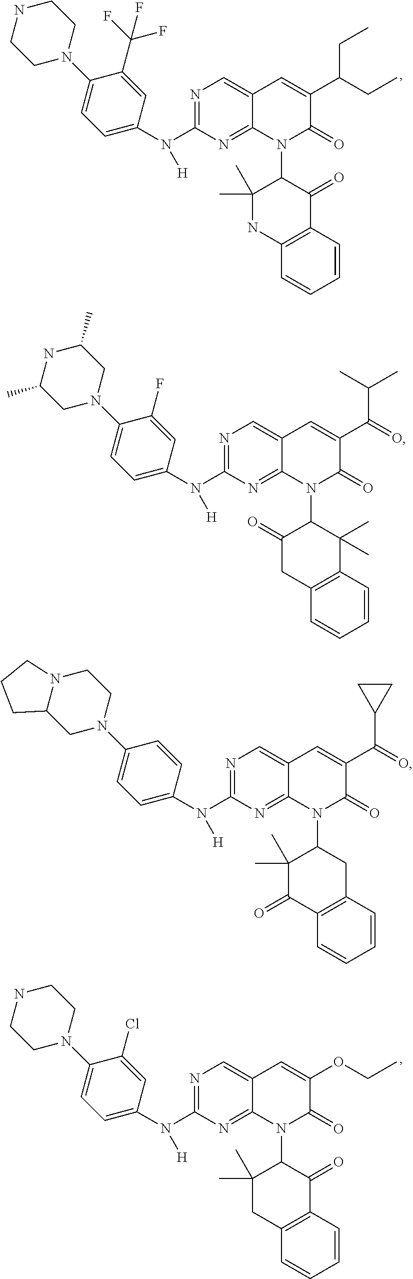

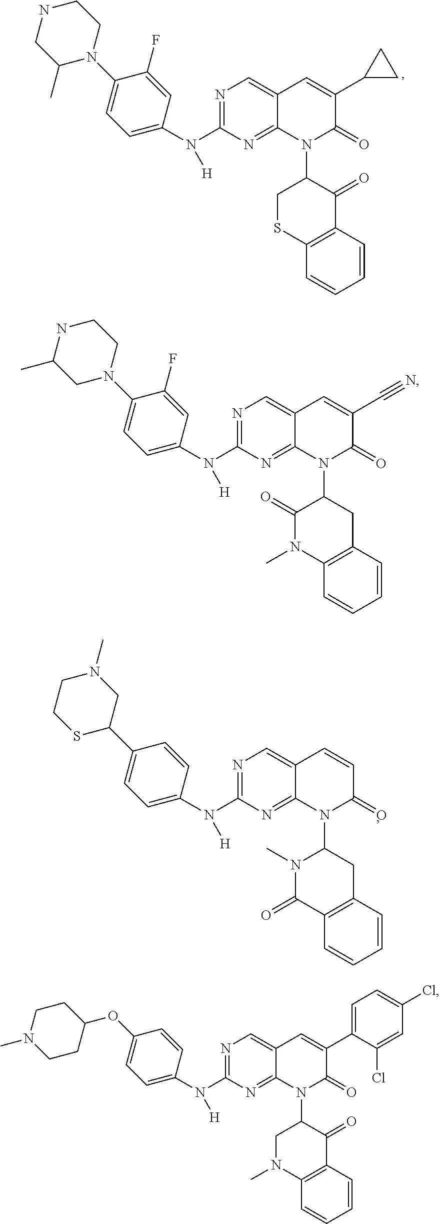

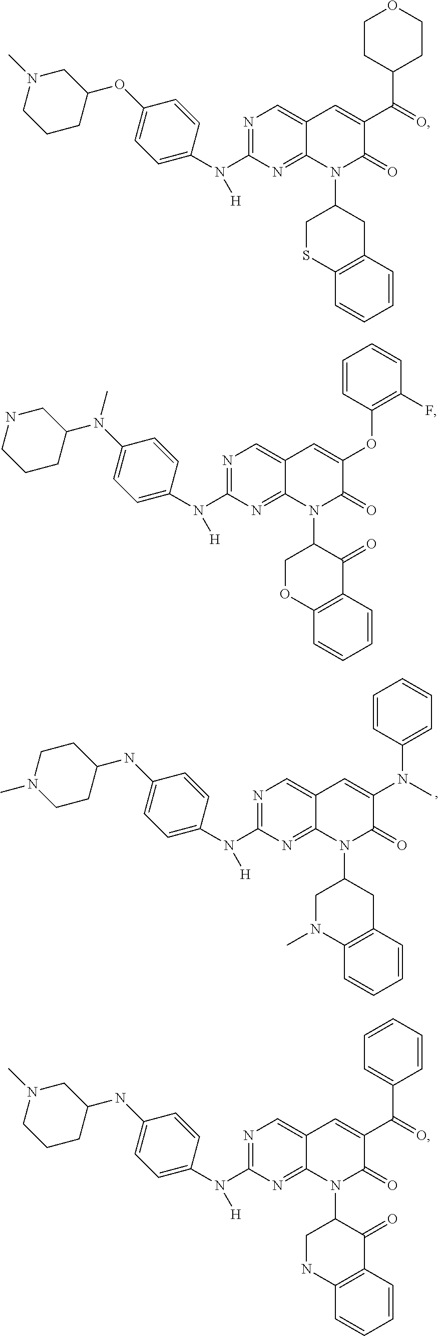

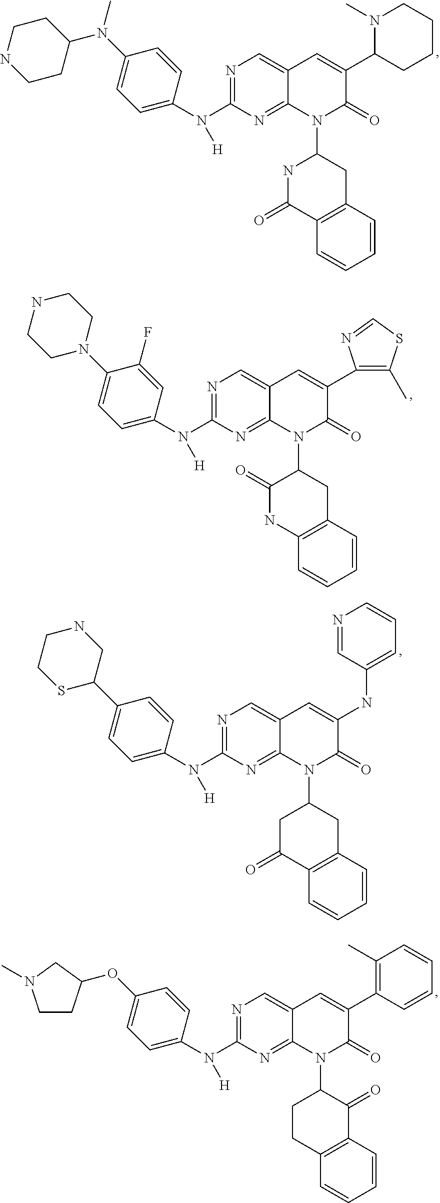

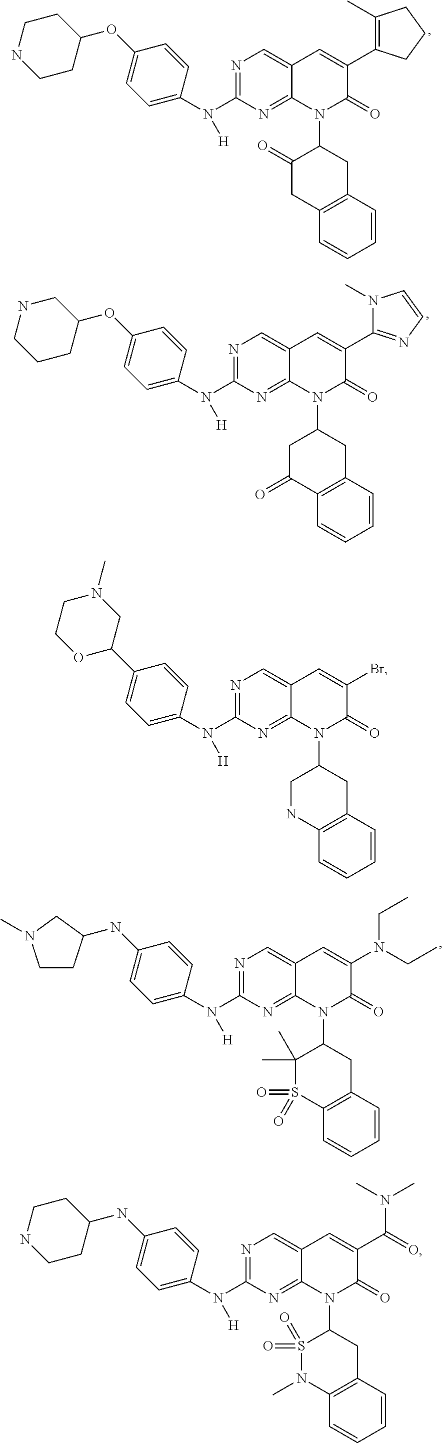

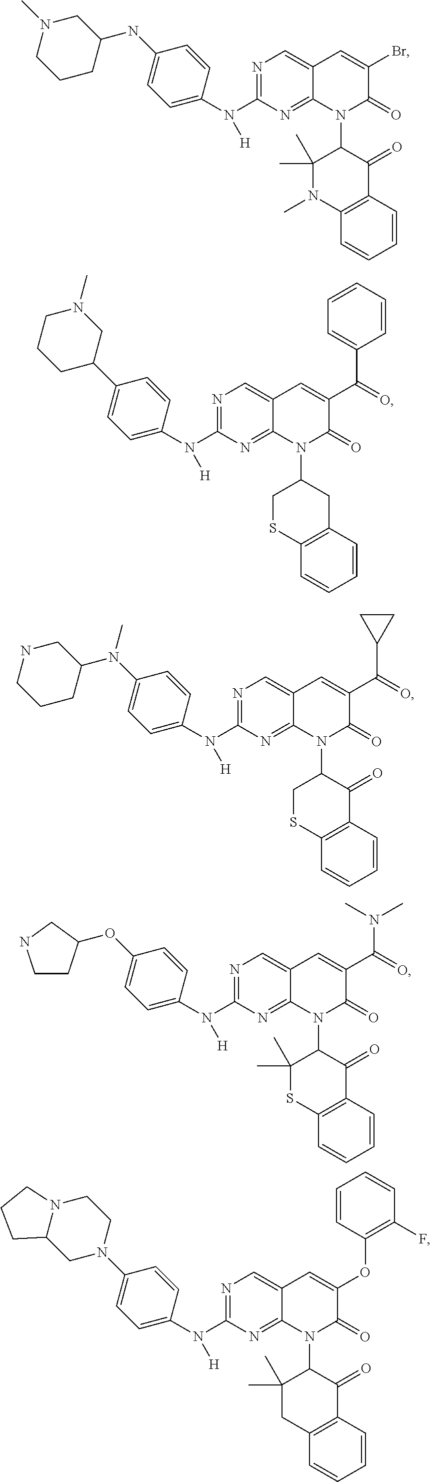

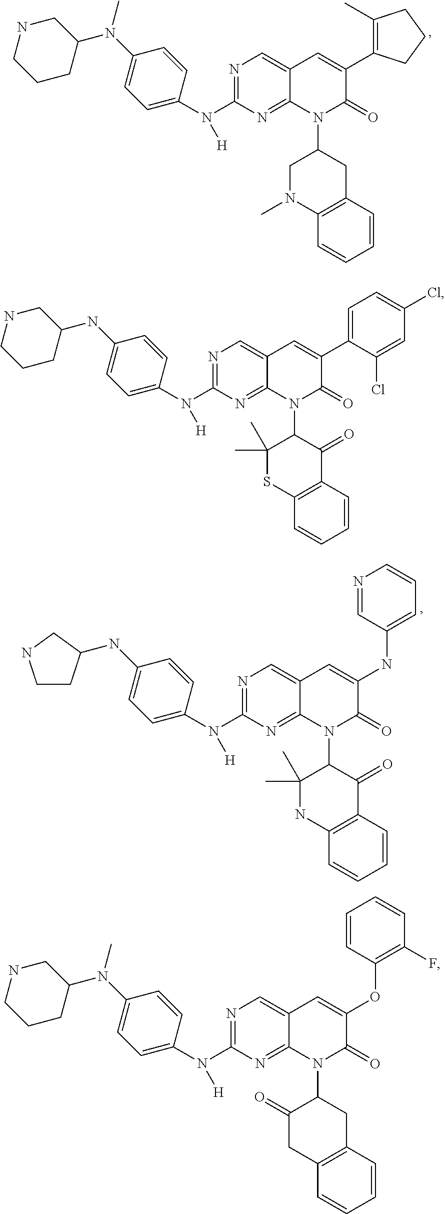

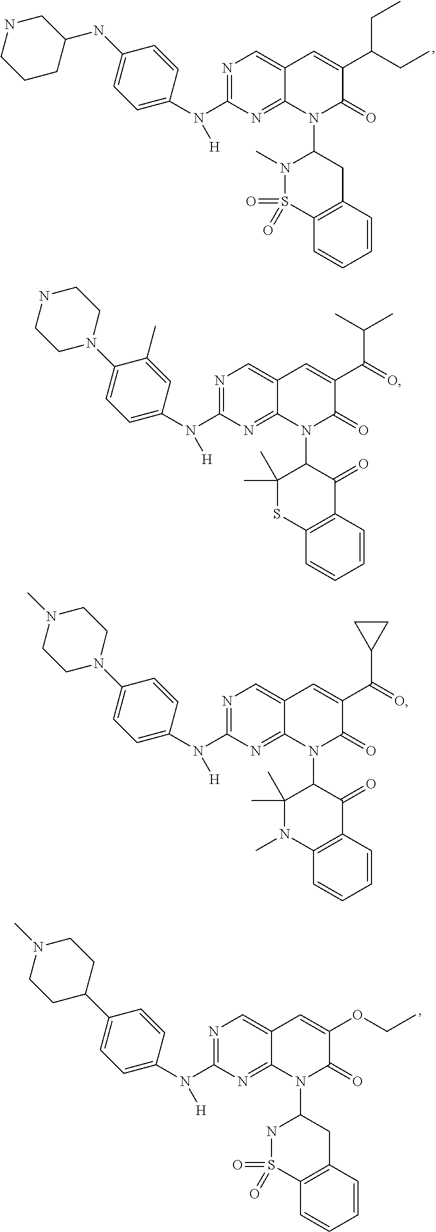

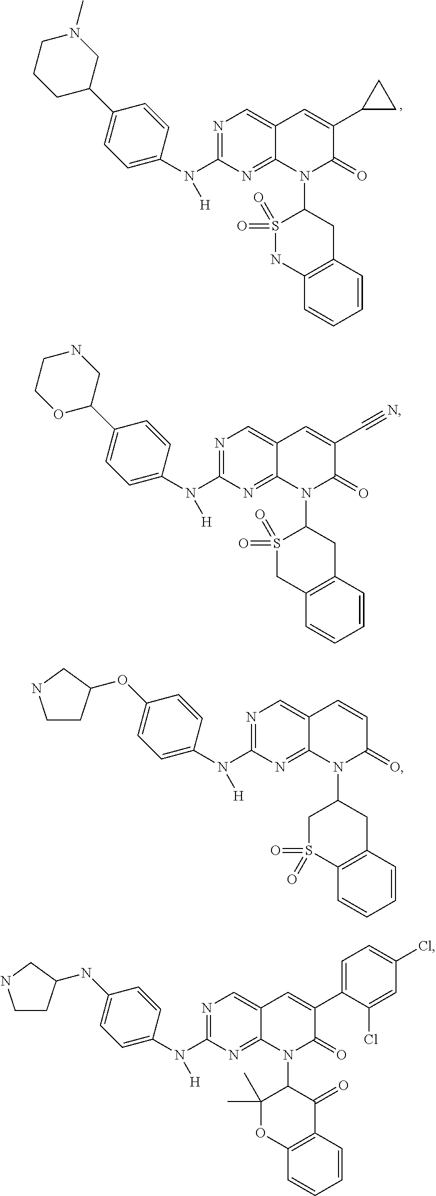

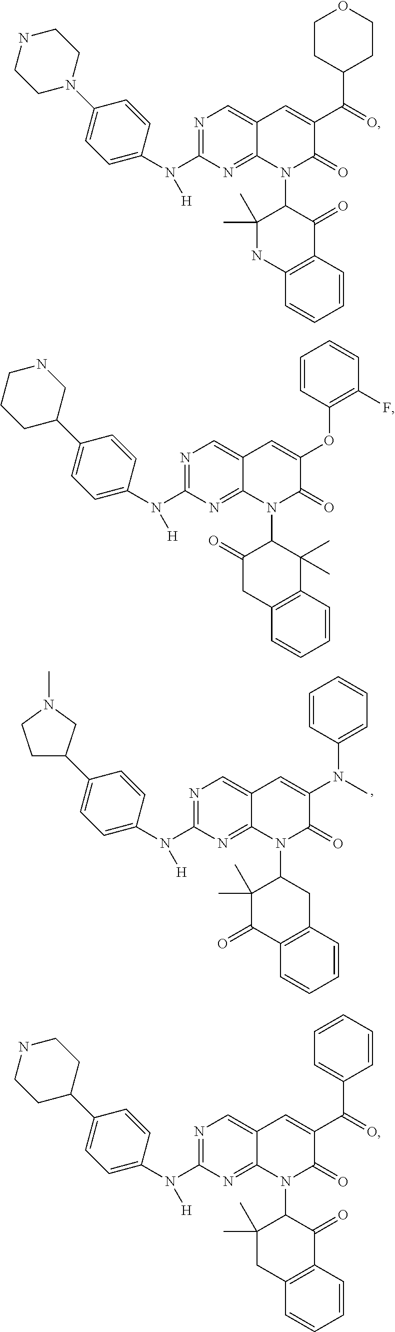

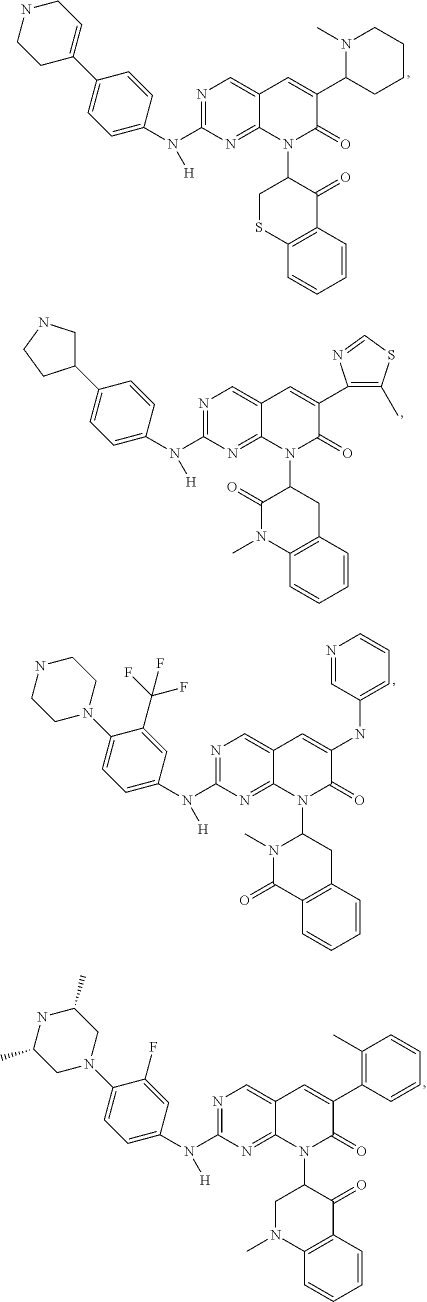

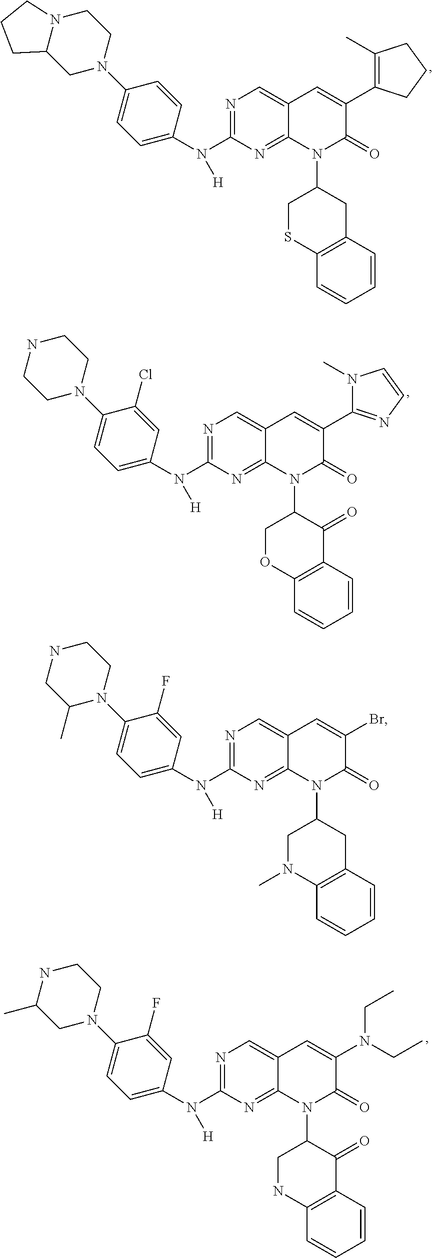 [SMILES: CC1=C(c2cc3cnc(Nc4ccc(N5CCN6CCCC6C5)cc4)nc3n(C3CSc4ccccc4C3)c2=O)CCC1 ; Cn1ccnc1-c1cc2cnc(Nc3ccc(N4CCNCC4)c(Cl)c3)nc2n(C2COc3ccccc3C2=O)c1=O ; CC1CNCCN1c1ccc(Nc2ncc3cc(Br)c(=O)n(C4Cc5ccccc5N(C)C4)c3n2)cc1F ; CCN(CC)c1cc2cnc(Nc3ccc(N4CCNC(C)C4)c(F)c3)nc2n(C2CNc3ccccc3C2=O)c1=O]

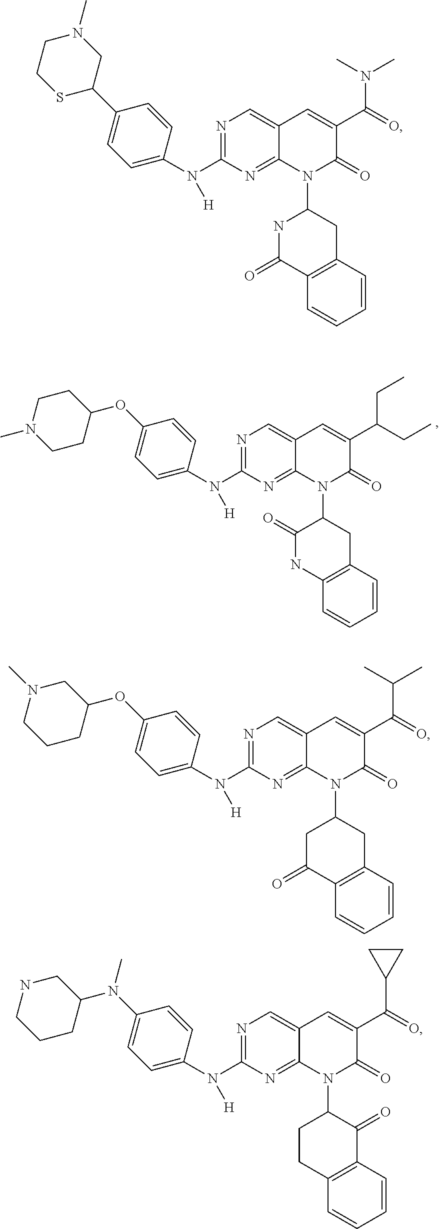

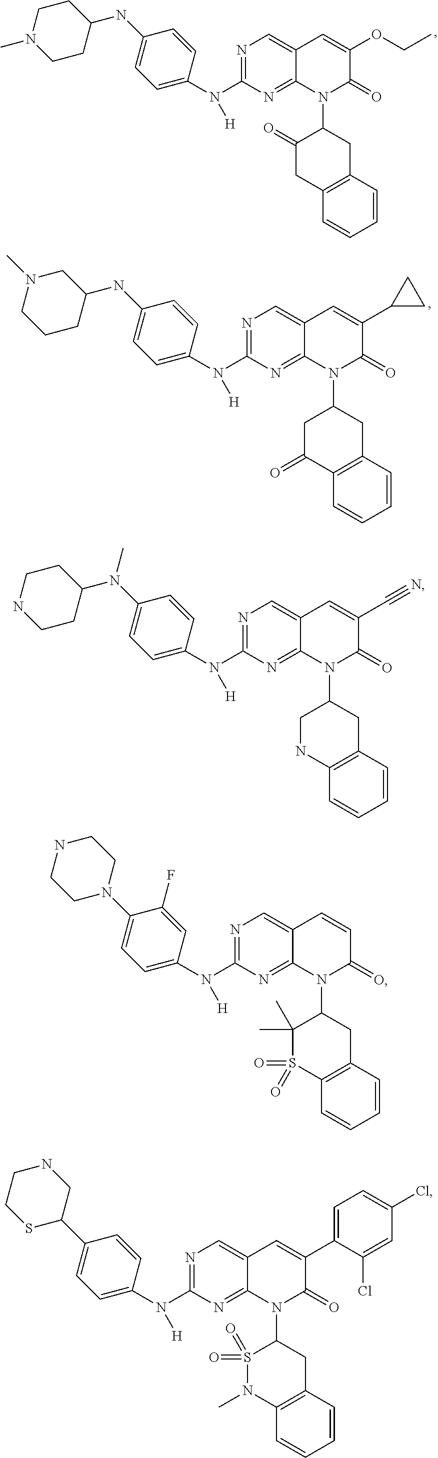

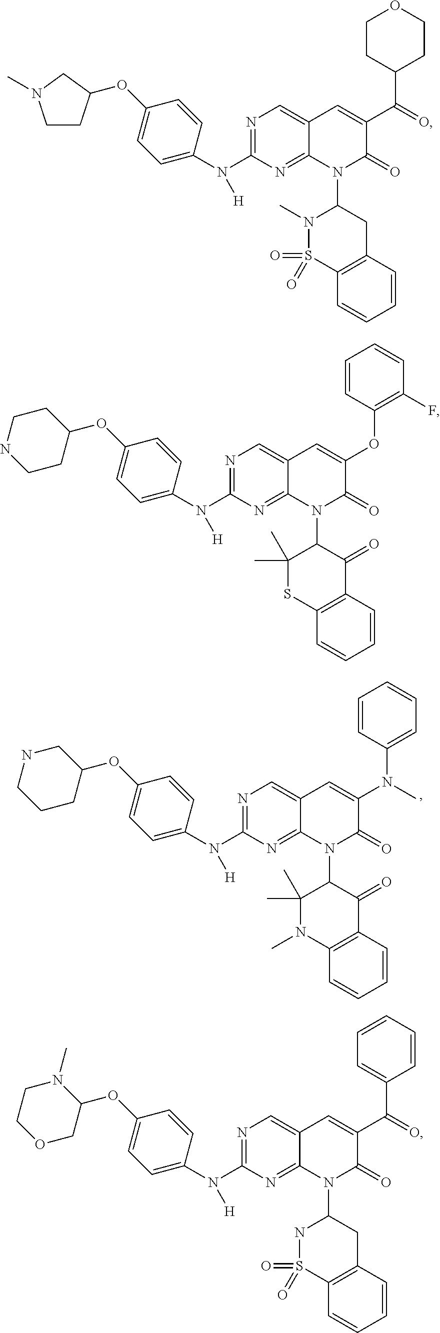

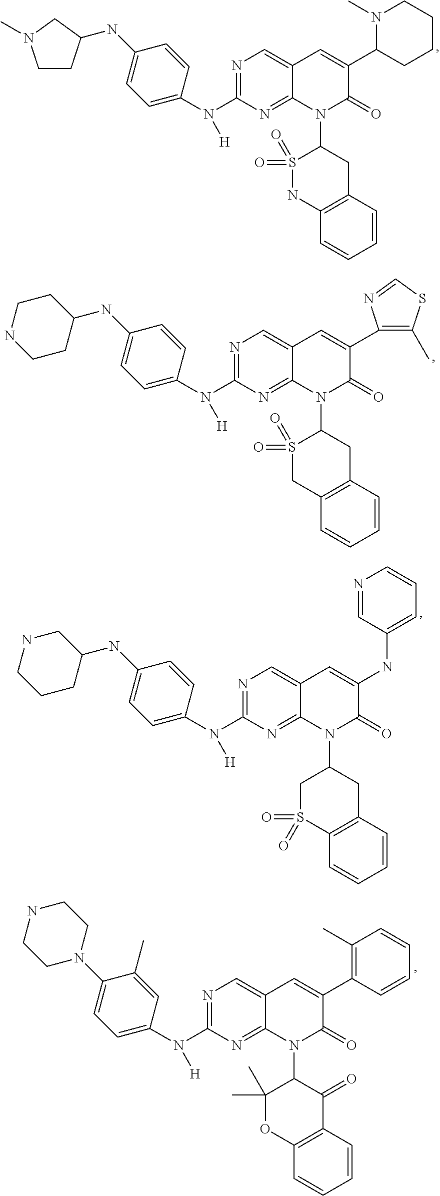

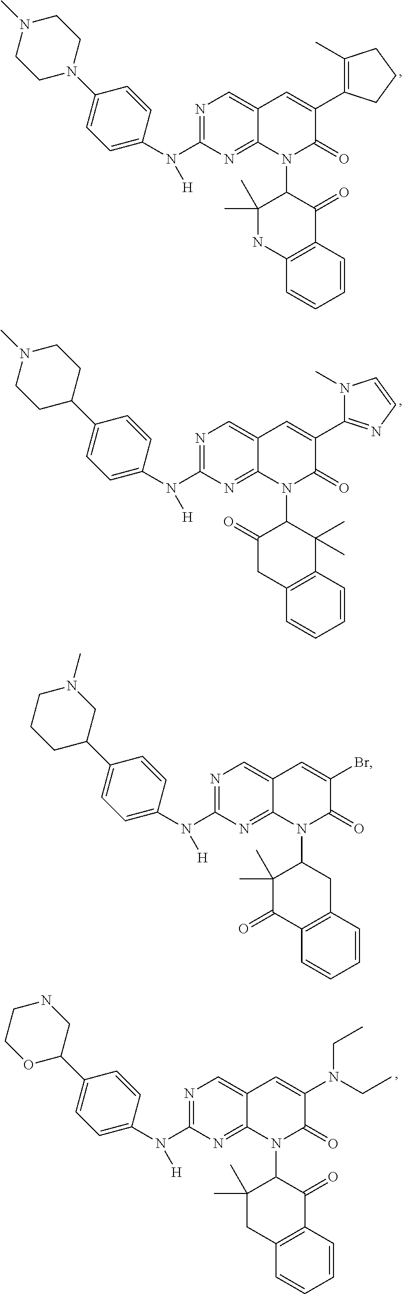 [SMILES: CC1=C(c2cc3cnc(Nc4ccc(N5CCN(C)CC5)cc4)nc3n(C3C(=O)c4ccccc4NC3(C)C)c2=O)CCC1 ; CN1CCC(c2ccc(Nc3ncc4cc(-c5nccn5C)c(=O)n(C5C(=O)Cc6ccccc6C5(C)C)c4n3)cc2)CC1 ; CN1CCCC(c2ccc(Nc3ncc4cc(Br)c(=O)n(C5Cc6ccccc6C(=O)C5(C)C)c4n3)cc2)C1 ; CCN(CC)c1cc2cnc(Nc3ccc(C4CNCCO4)cc3)nc2n(C2C(=O)c3ccccc3CC2(C)C)c1=O]

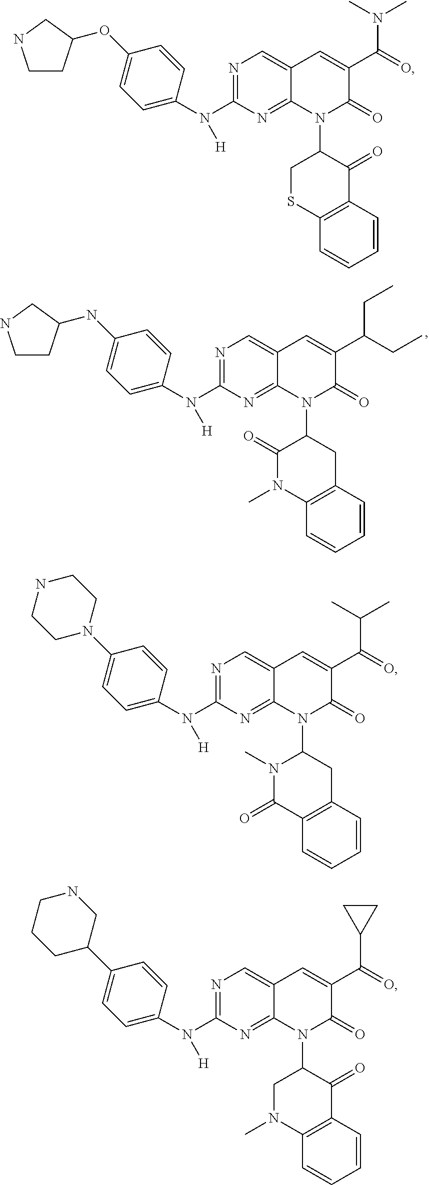

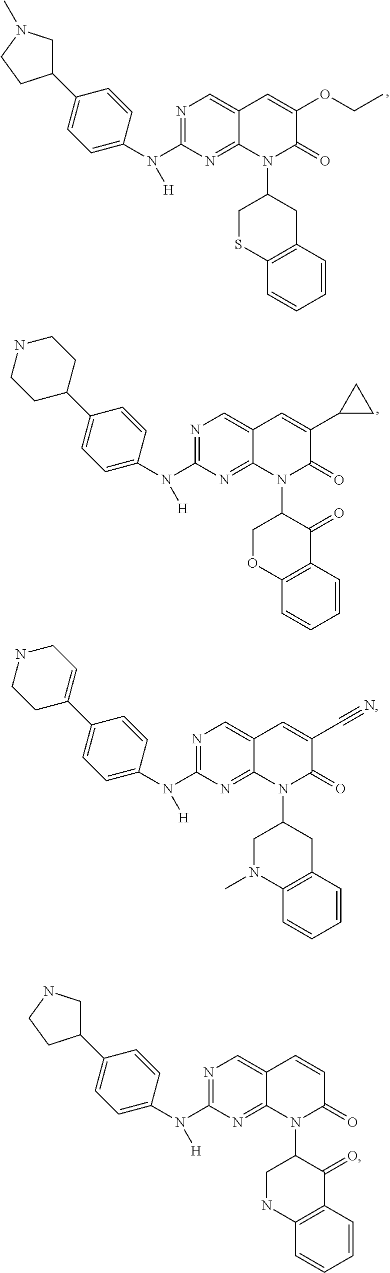 [SMILES: CCOc1cc2cnc(Nc3ccc(C4CCN(C)C4)cc3)nc2n(C2CSc3ccccc3C2)c1=O ; O=C1c2ccccc2OCC1n1c(=O)c(C2CC2)cc2cnc(Nc3ccc(C4CCNCC4)cc3)nc21 ; CN1CC(n2c(=O)c(C#N)cc3cnc(Nc4ccc(C5=CCNCC5)cc4)nc32)Cc2ccccc21 ; O=C1c2ccccc2NCC1n1c(=O)ccc2cnc(Nc3ccc(C4CCNC4)cc3)nc21]

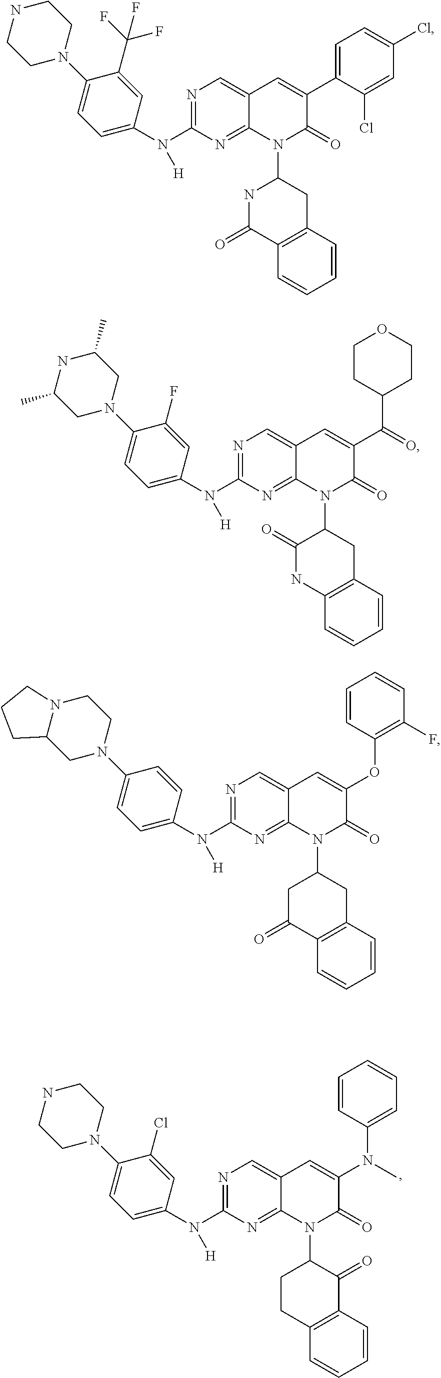

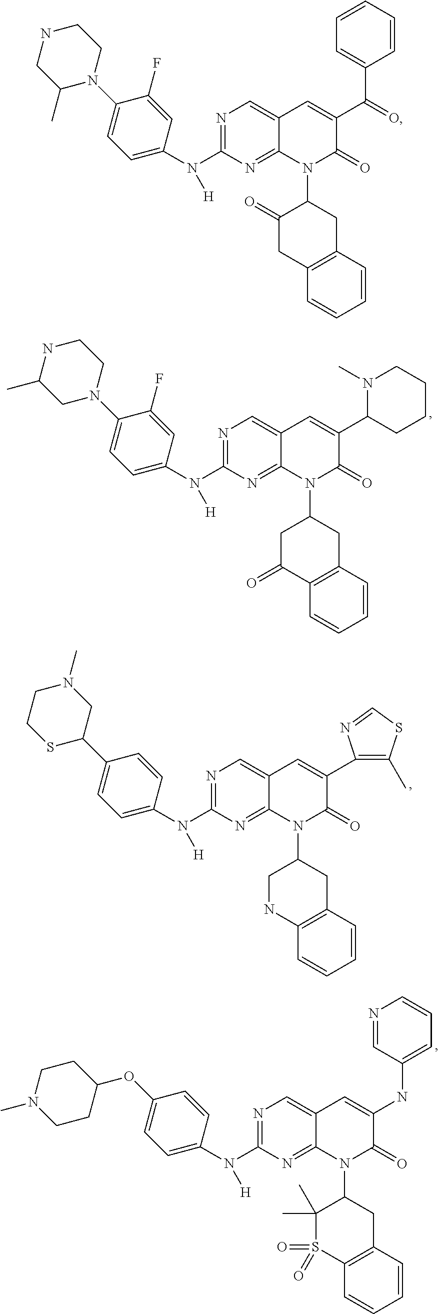

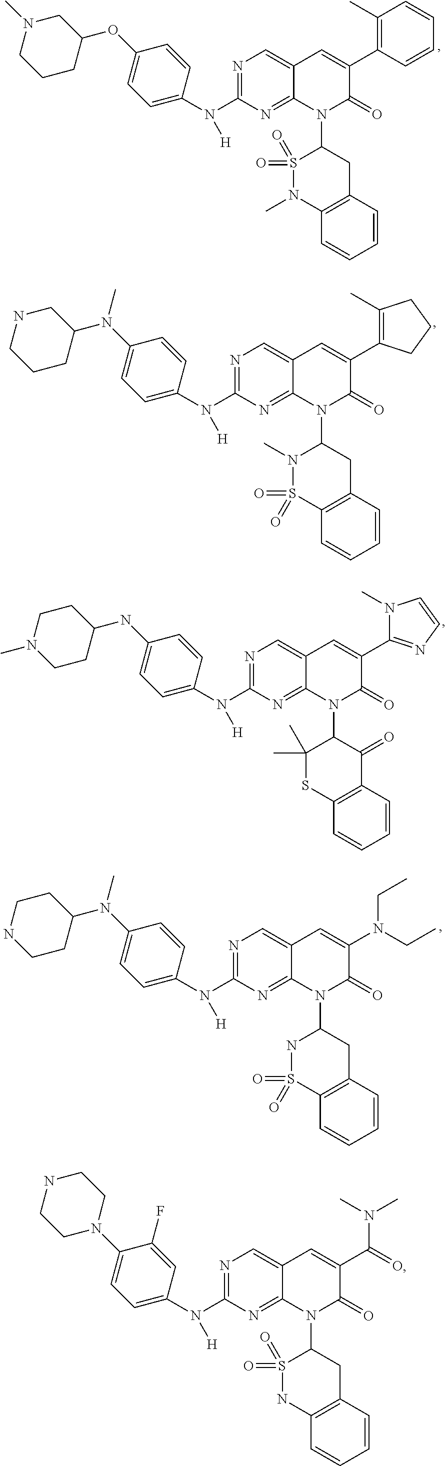

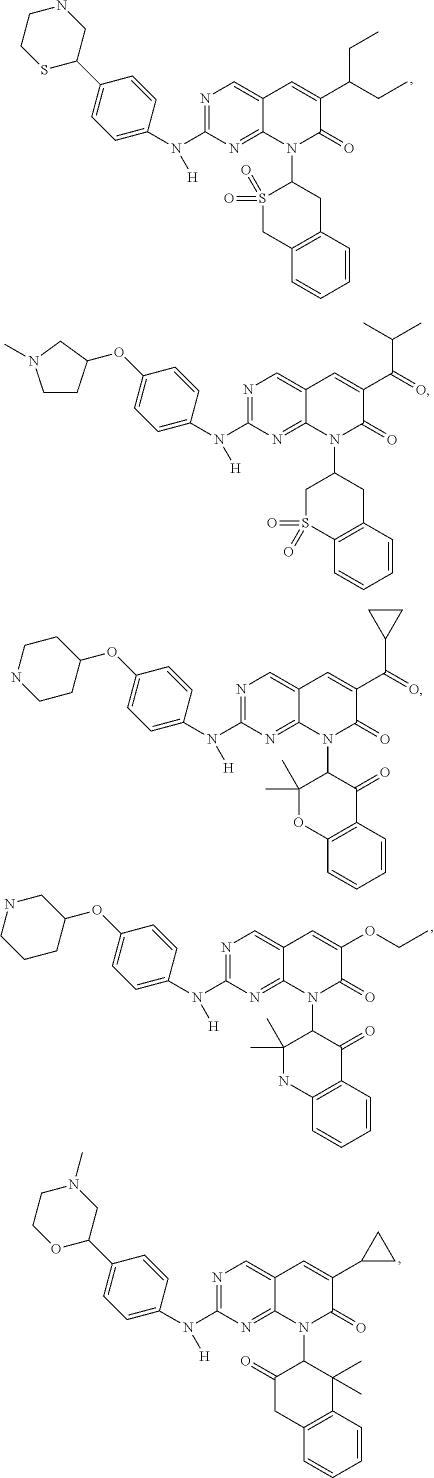

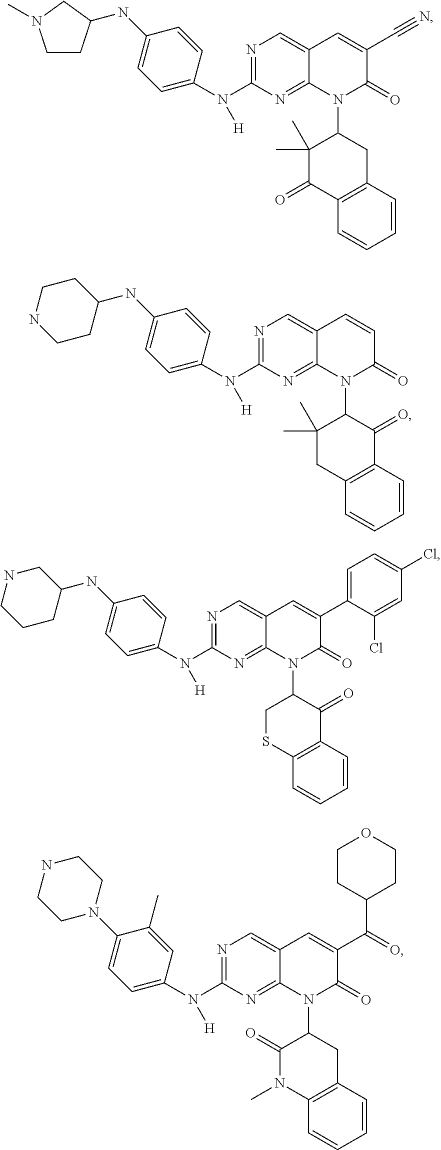

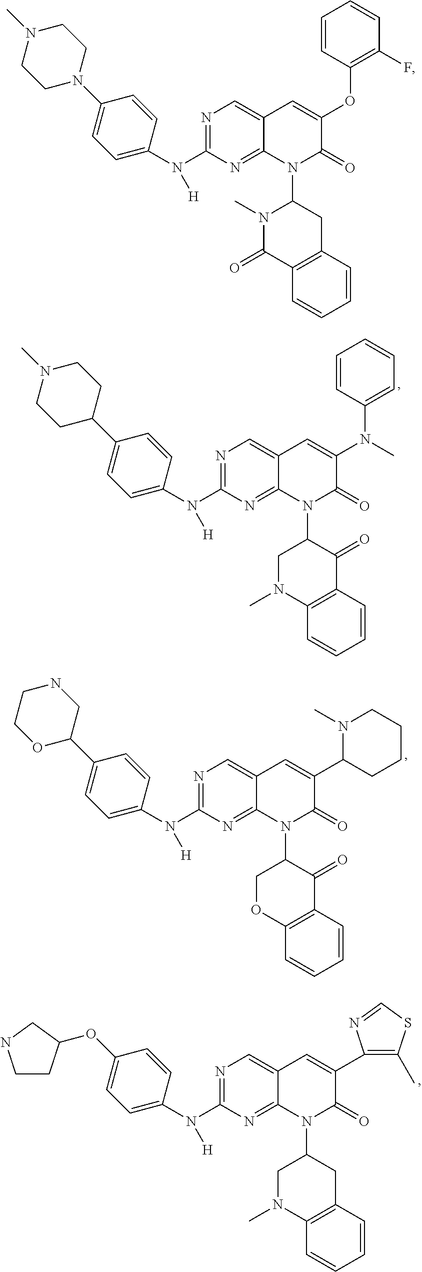

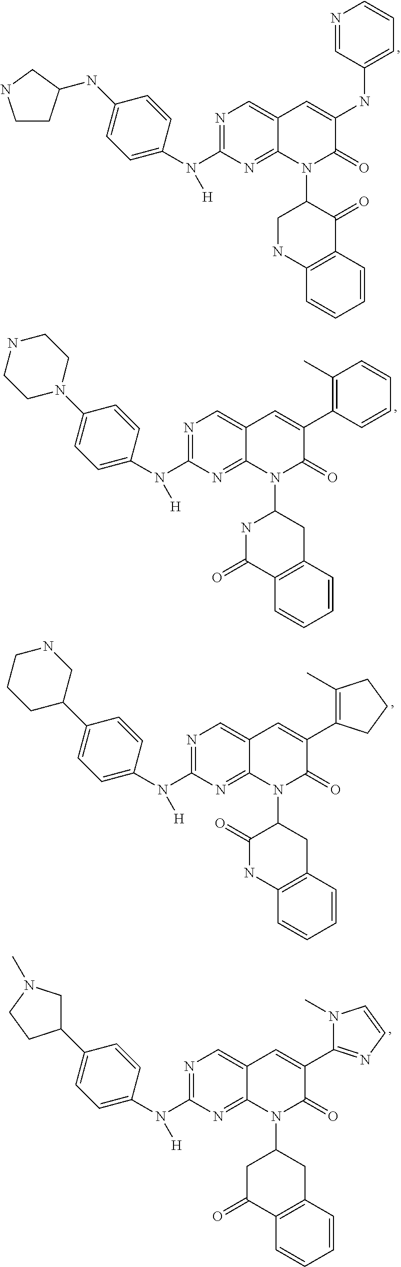

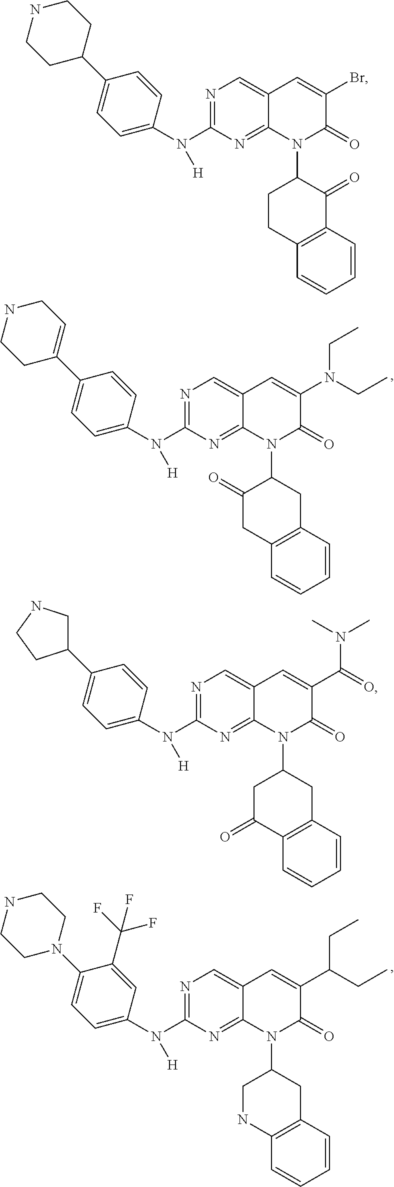

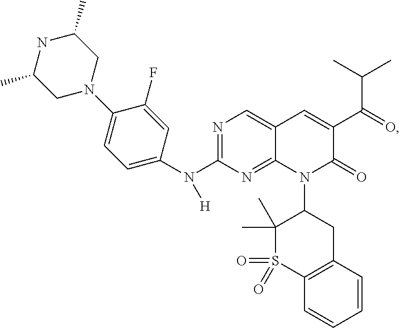 [SMILES: CC(C)C(=O)c1cc2cnc(Nc3ccc(N4C[C@@H](C)N[C@@H](C)C4)c(F)c3)nc2n(C2Cc3ccccc3S(=O)(=O)C2(C)C)c1=O]

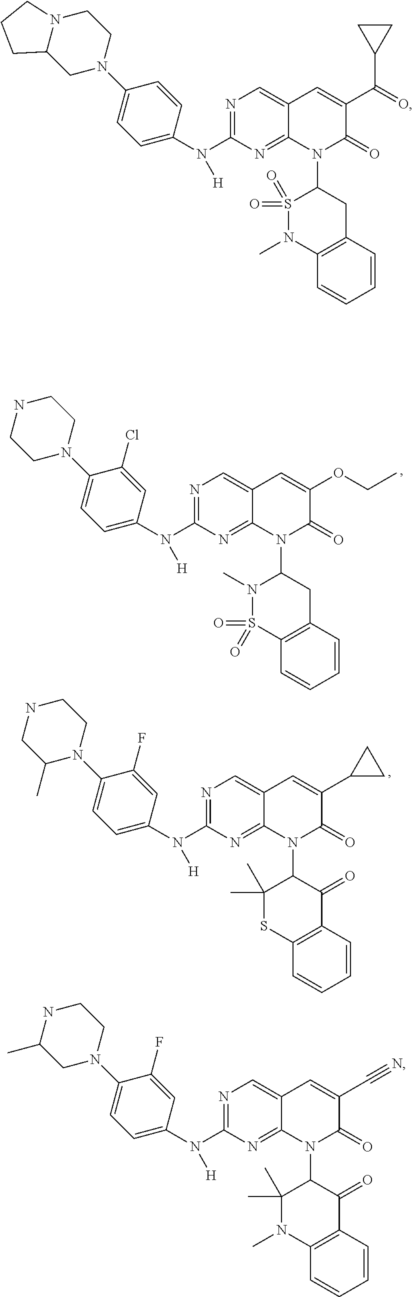

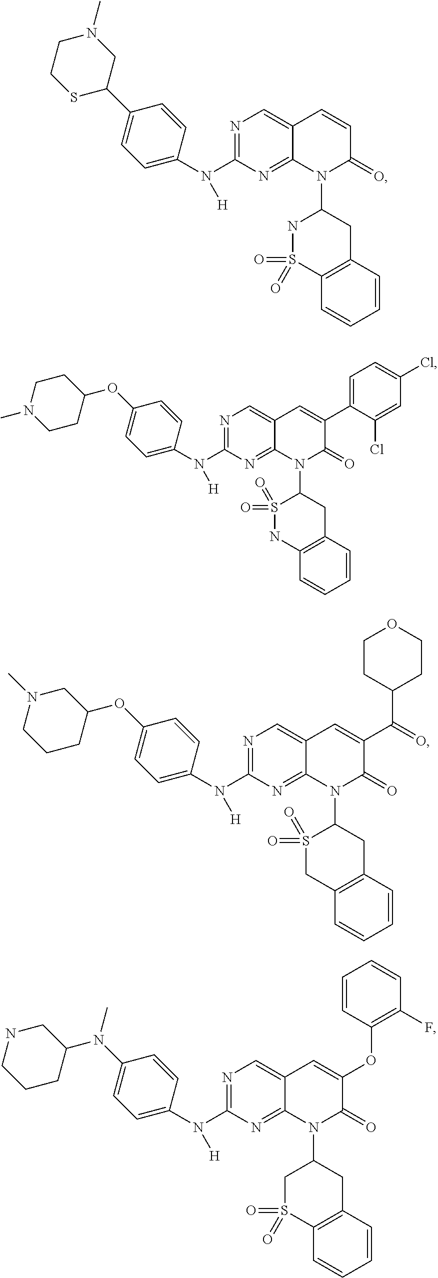

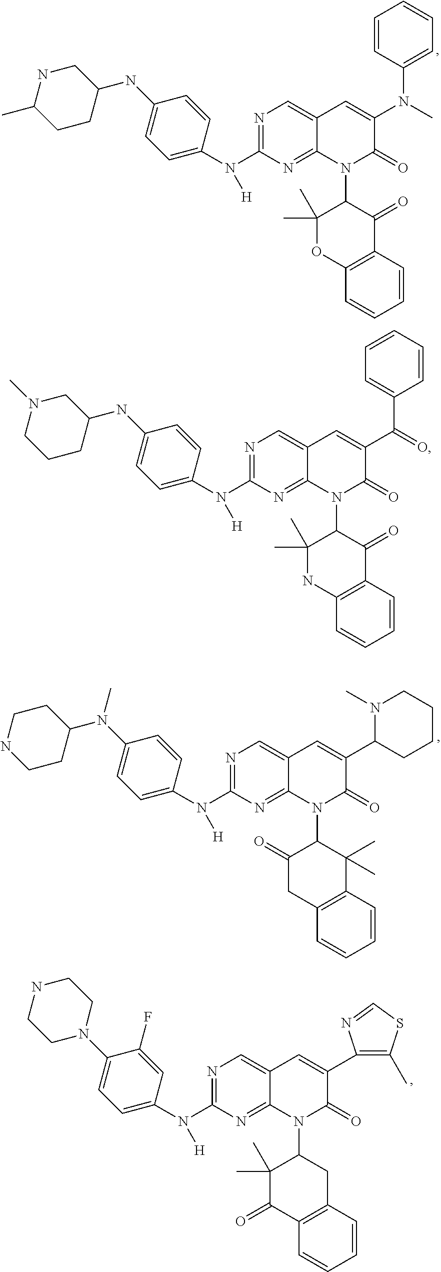

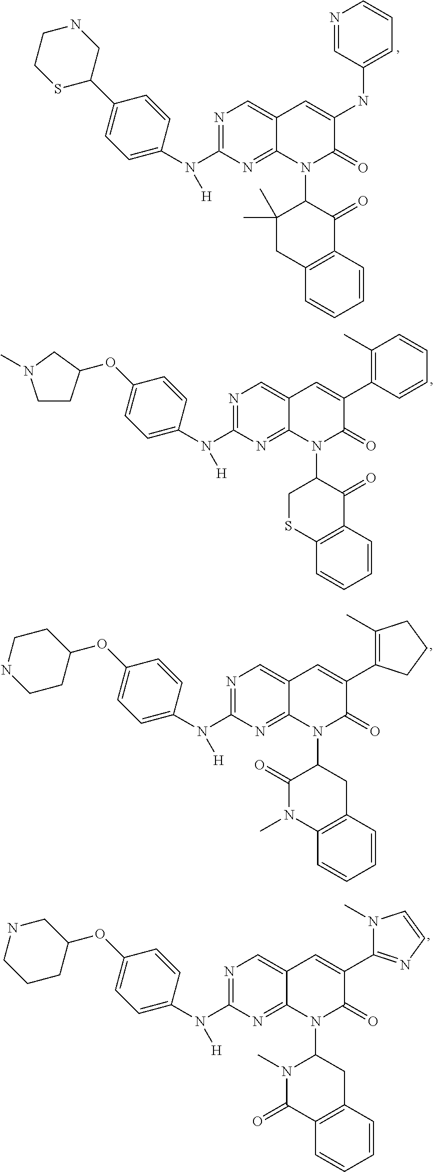

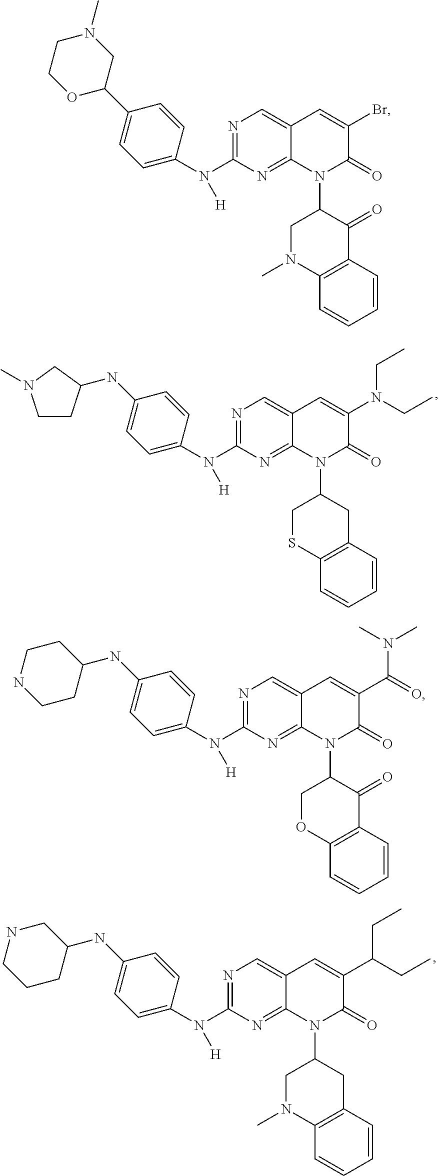

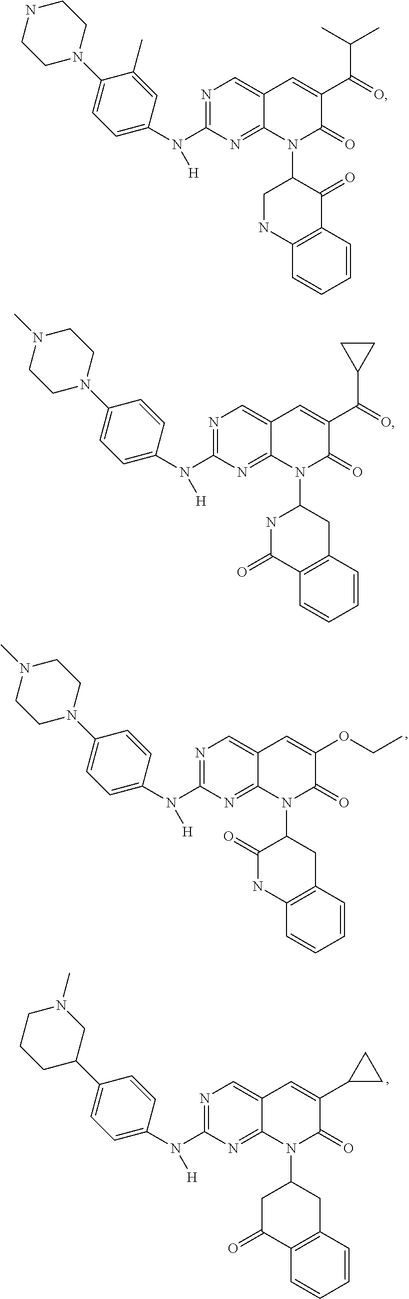

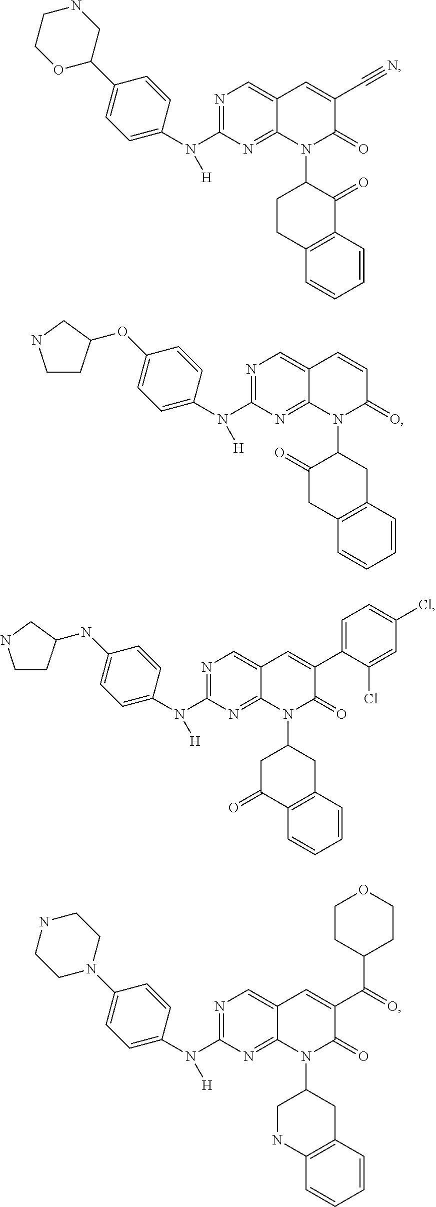

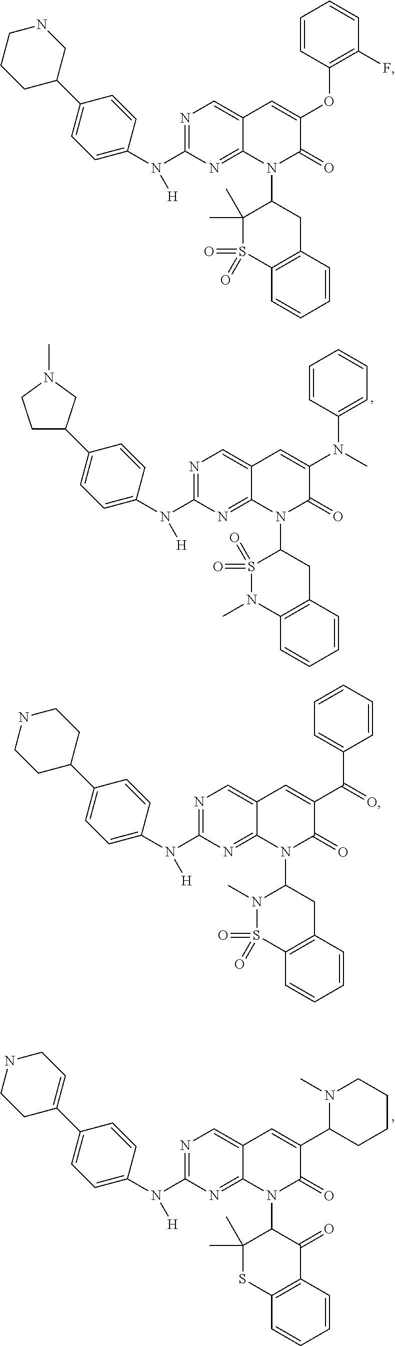

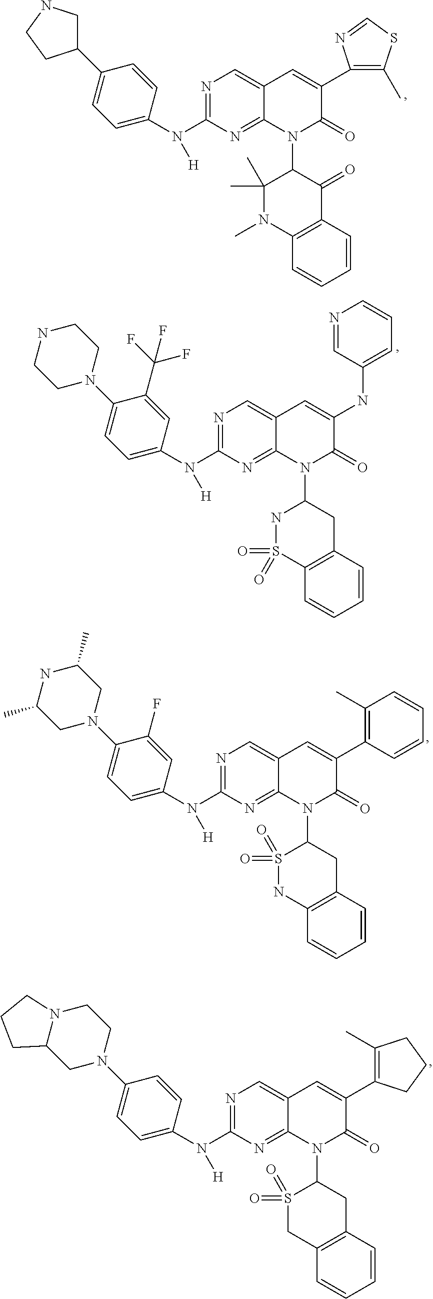

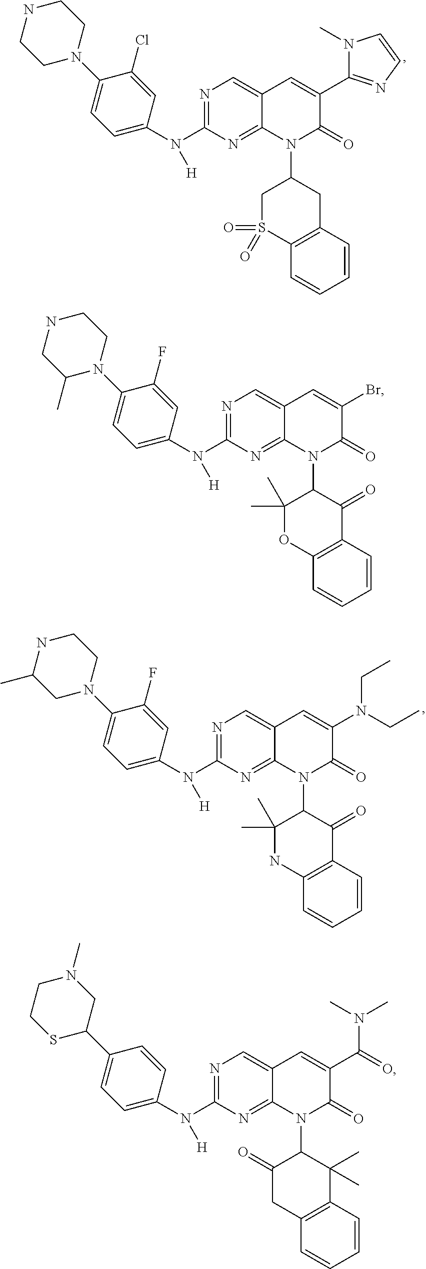 [SMILES: Cn1ccnc1-c1cc2cnc(Nc3ccc(N4CCNCC4)c(Cl)c3)nc2n(C2Cc3ccccc3S(=O)(=O)C2)c1=O ; CC1CNCCN1c1ccc(Nc2ncc3cc(Br)c(=O)n(C4C(=O)c5ccccc5OC4(C)C)c3n2)cc1F ; CCN(CC)c1cc2cnc(Nc3ccc(N4CCNC(C)C4)c(F)c3)nc2n(C2C(=O)c3ccccc3NC2(C)C)c1=O ; CN1CCSC(c2ccc(Nc3ncc4cc(C(=O)N(C)C)c(=O)n(C5C(=O)Cc6ccccc6C5(C)C)c4n3)cc2)C1]

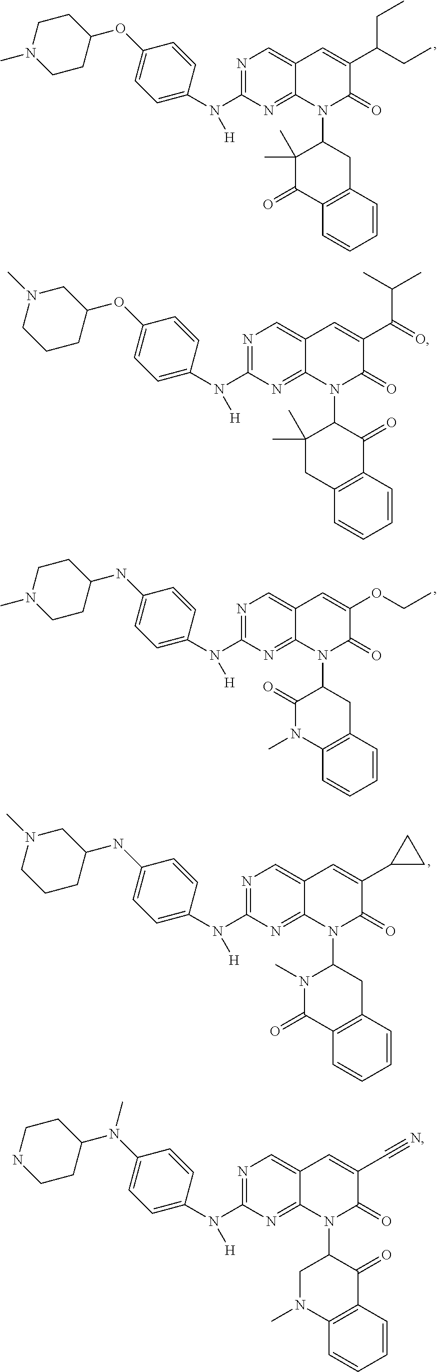

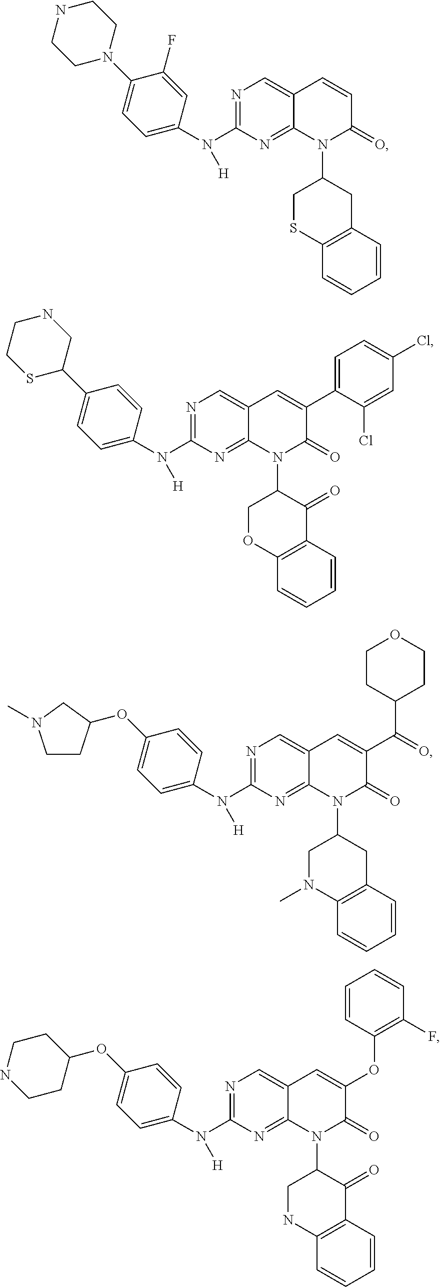

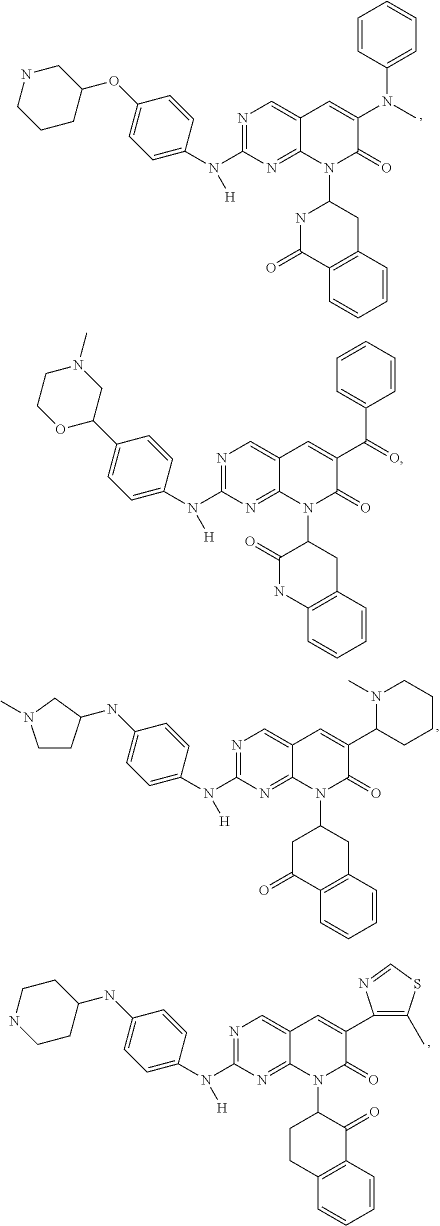

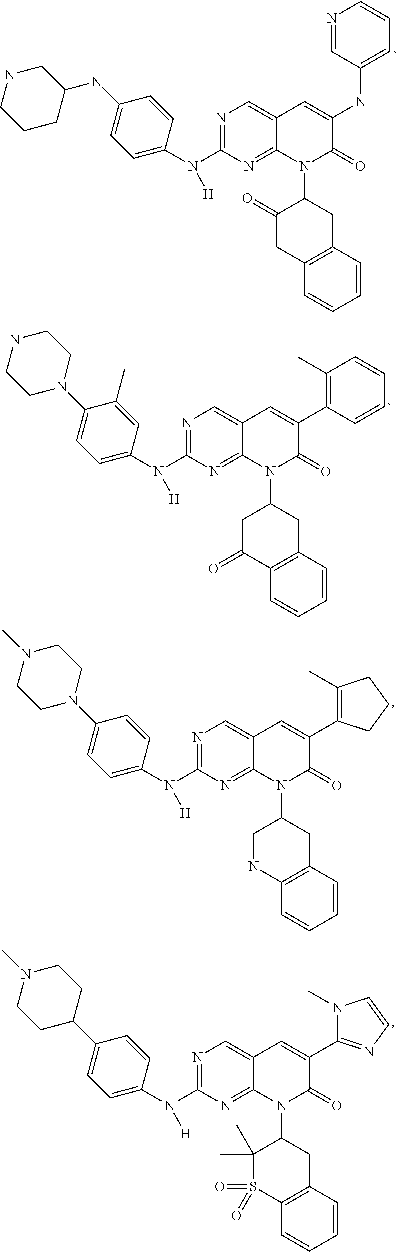 [SMILES: O=C1Cc2ccccc2CC1n1c(=O)c(Nc2cccnc2)cc2cnc(Nc3ccc(NC4CCCNC4)cc3)nc21 ; Cc1ccccc1-c1cc2cnc(Nc3ccc(N4CCNCC4)c(C)c3)nc2n(C2CC(=O)c3ccccc3C2)c1=O ; CC1=C(c2cc3cnc(Nc4ccc(N5CCN(C)CC5)cc4)nc3n(C3CNc4ccccc4C3)c2=O)CCC1 ; CN1CCC(c2ccc(Nc3ncc4cc(-c5nccn5C)c(=O)n(C5Cc6ccccc6S(=O)(=O)C5(C)C)c4n3)cc2)CC1]

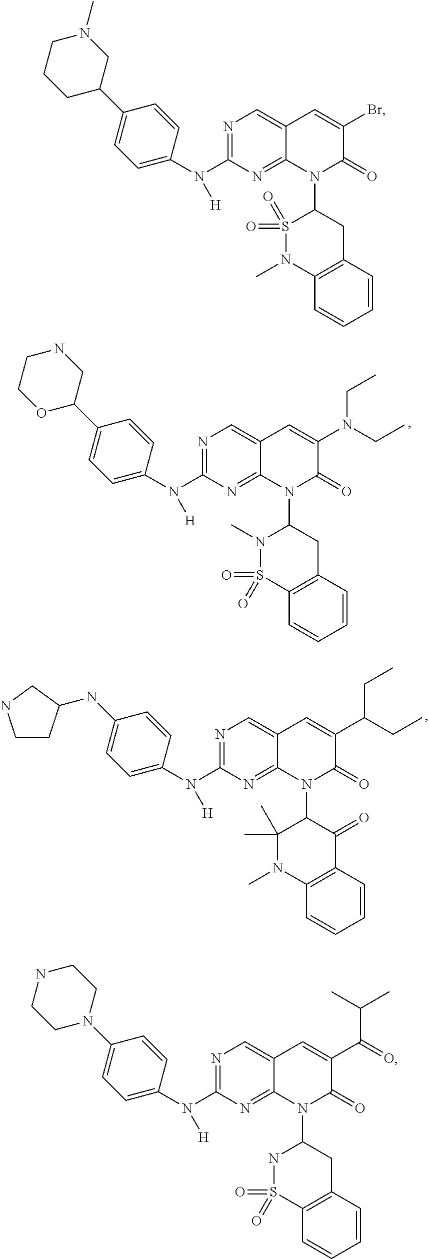 [SMILES: CN1CCCC(c2ccc(Nc3ncc4cc(Br)c(=O)n(C5Cc6ccccc6N(C)S5(=O)=O)c4n3)cc2)C1 ; CCN(CC)c1cc2cnc(Nc3ccc(C4CNCCO4)cc3)nc2n(C2Cc3ccccc3S(=O)(=O)N2C)c1=O ; CCC(CC)c1cc2cnc(Nc3ccc(NC4CCNC4)cc3)nc2n(C2C(=O)c3ccccc3N(C)C2(C)C)c1=O ; CC(C)C(=O)c1cc2cnc(Nc3ccc(N4CCNCC4)cc3)nc2n(C2Cc3ccccc3S(=O)(=O)N2)c1=O]

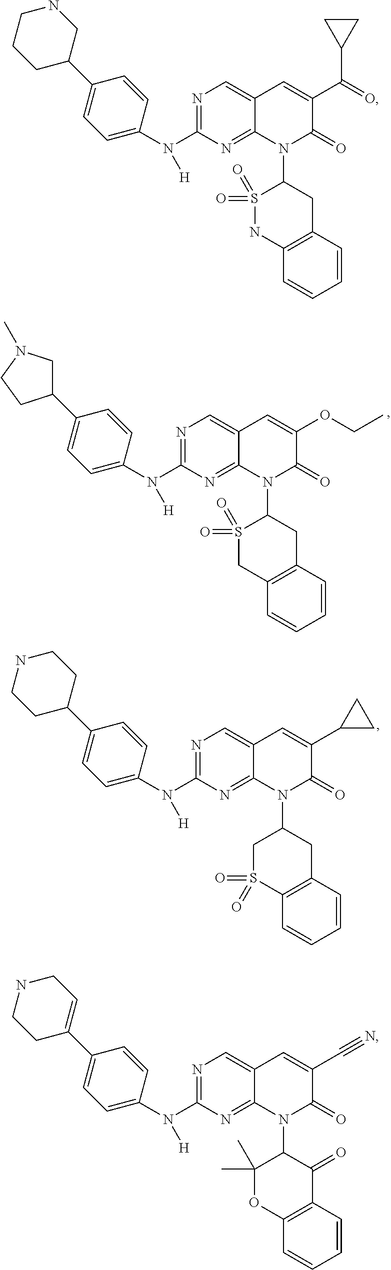 [SMILES: O=C(c1cc2cnc(Nc3ccc(C4CCCNC4)cc3)nc2n(C2Cc3ccccc3NS2(=O)=O)c1=O)C1CC1 ; CCOc1cc2cnc(Nc3ccc(C4CCN(C)C4)cc3)nc2n(C2Cc3ccccc3CS2(=O)=O)c1=O ; O=c1c(C2CC2)cc2cnc(Nc3ccc(C4CCNCC4)cc3)nc2n1C1Cc2ccccc2S(=O)(=O)C1 ; CC1(C)Oc2ccccc2C(=O)C1n1c(=O)c(C#N)cc2cnc(Nc3ccc(C4=CCNCC4)cc3)nc21]

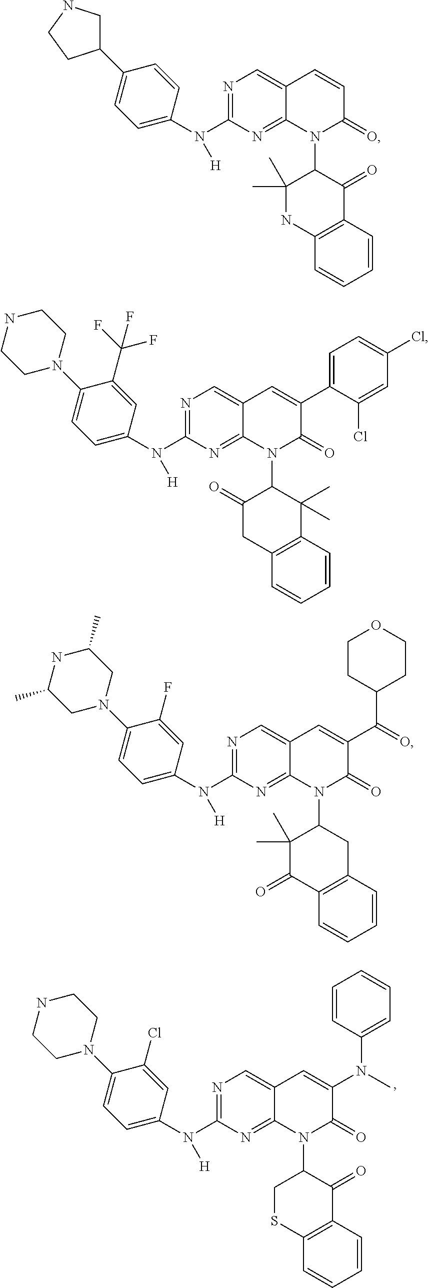

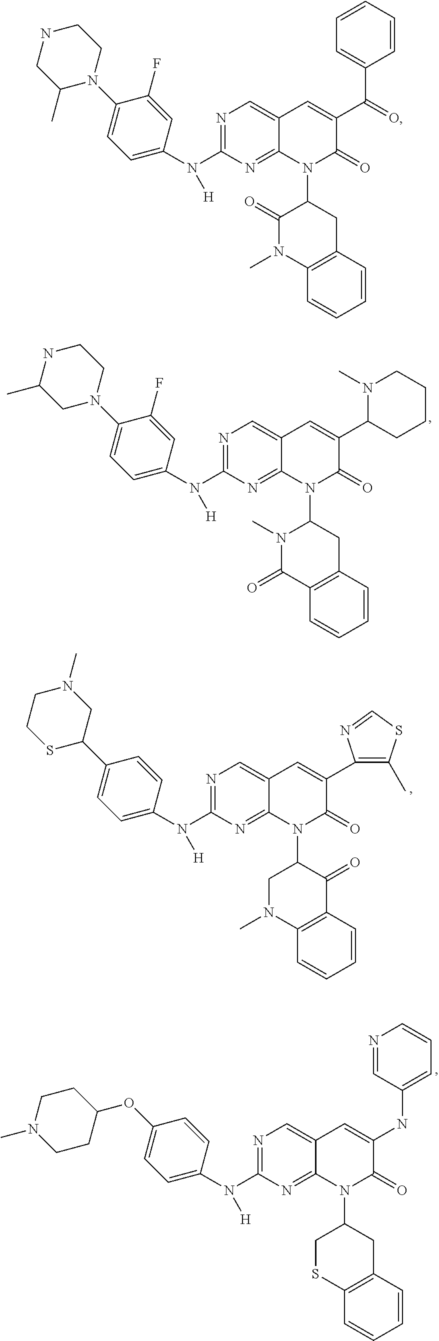

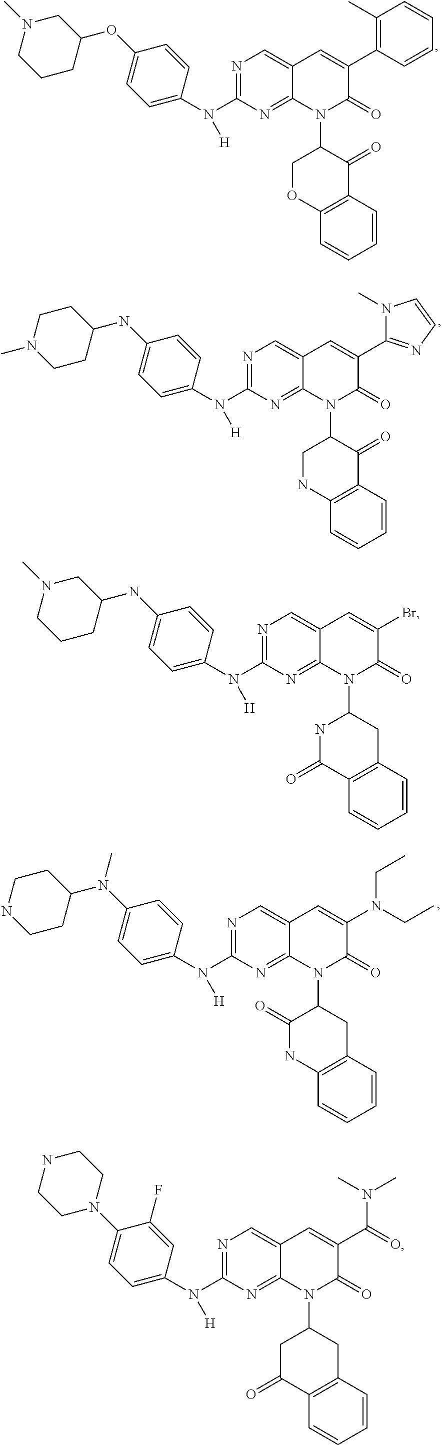

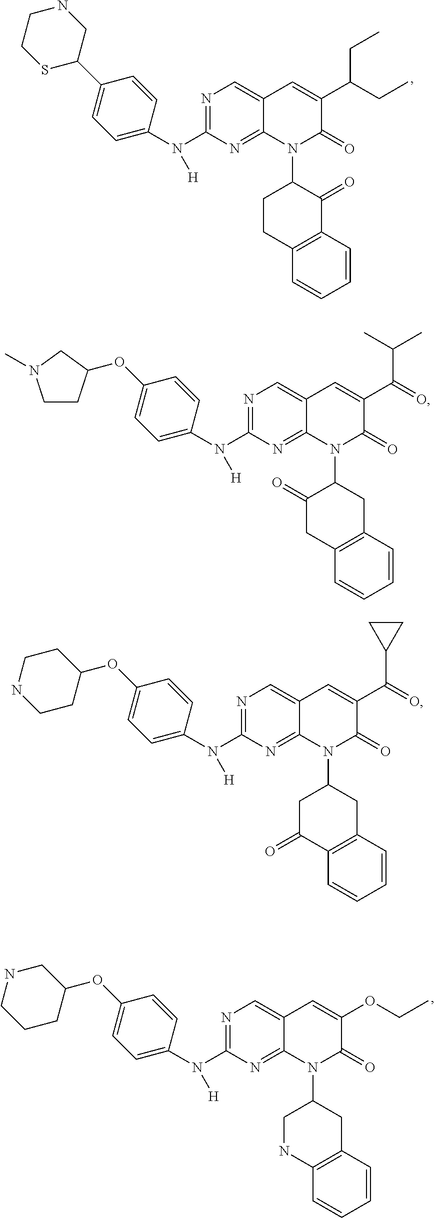

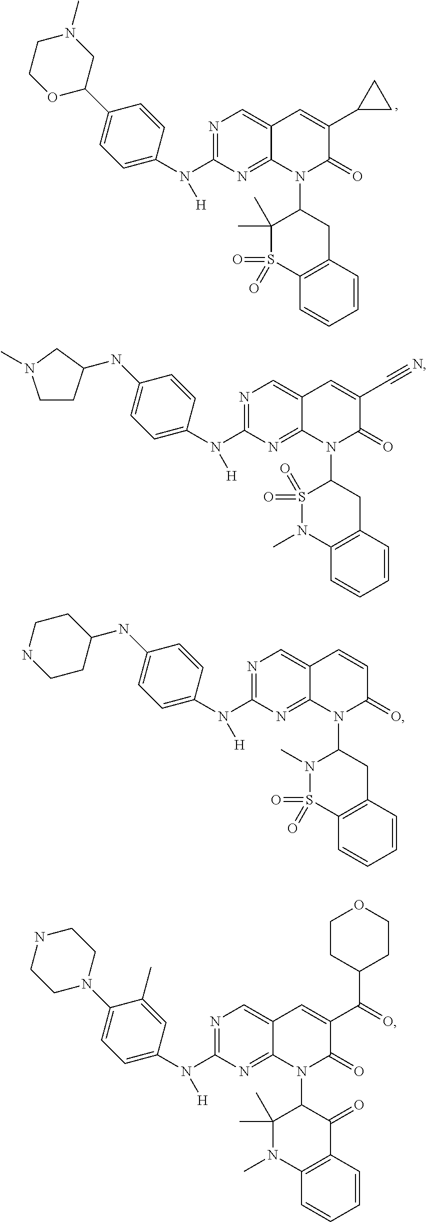

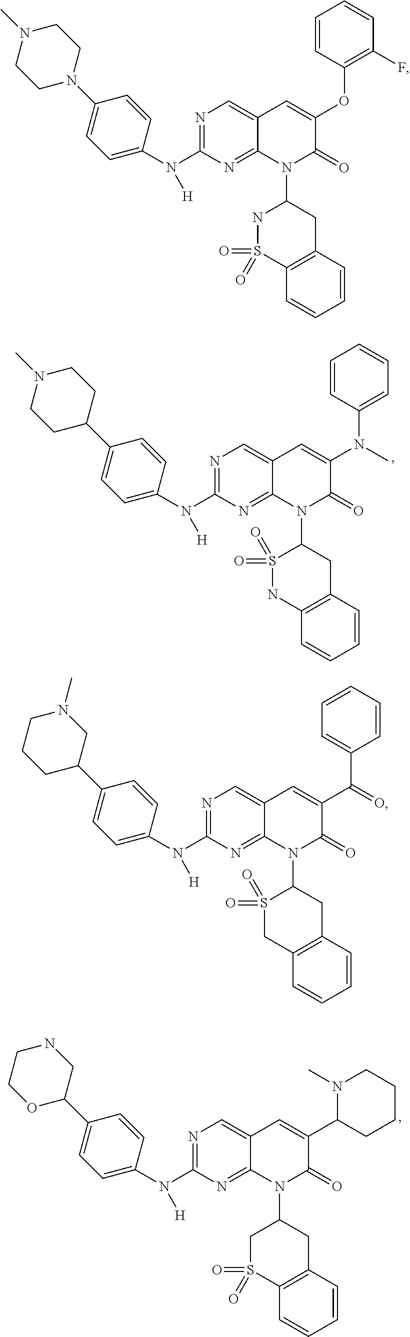

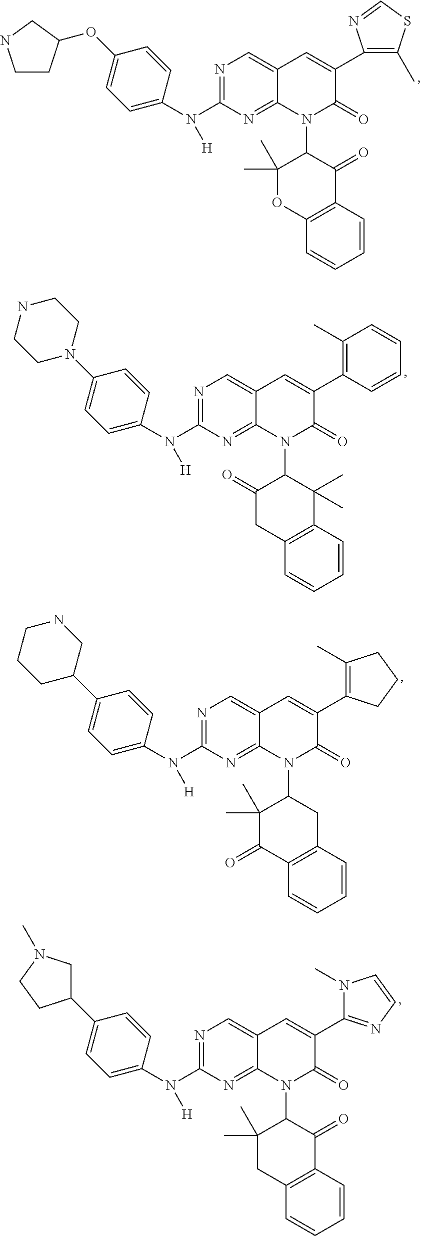 [SMILES: Cc1scnc1-c1cc2cnc(Nc3ccc(OC4CCNC4)cc3)nc2n(C2C(=O)c3ccccc3OC2(C)C)c1=O ; Cc1ccccc1-c1cc2cnc(Nc3ccc(N4CCNCC4)cc3)nc2n(C2C(=O)Cc3ccccc3C2(C)C)c1=O ; CC1=C(c2cc3cnc(Nc4ccc(C5CCCNC5)cc4)nc3n(C3Cc4ccccc4C(=O)C3(C)C)c2=O)CCC1 ; CN1CCC(c2ccc(Nc3ncc4cc(-c5nccn5C)c(=O)n(C5C(=O)c6ccccc6CC5(C)C)c4n3)cc2)C1]

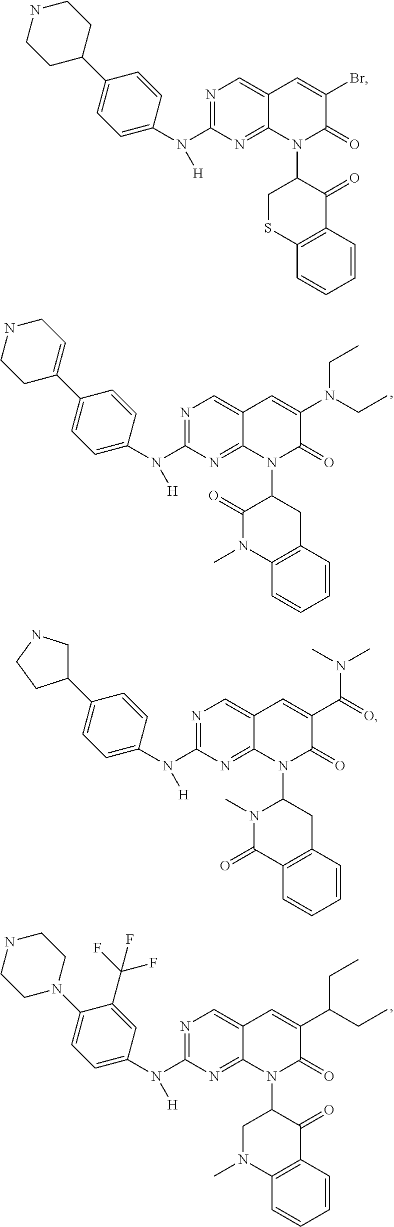

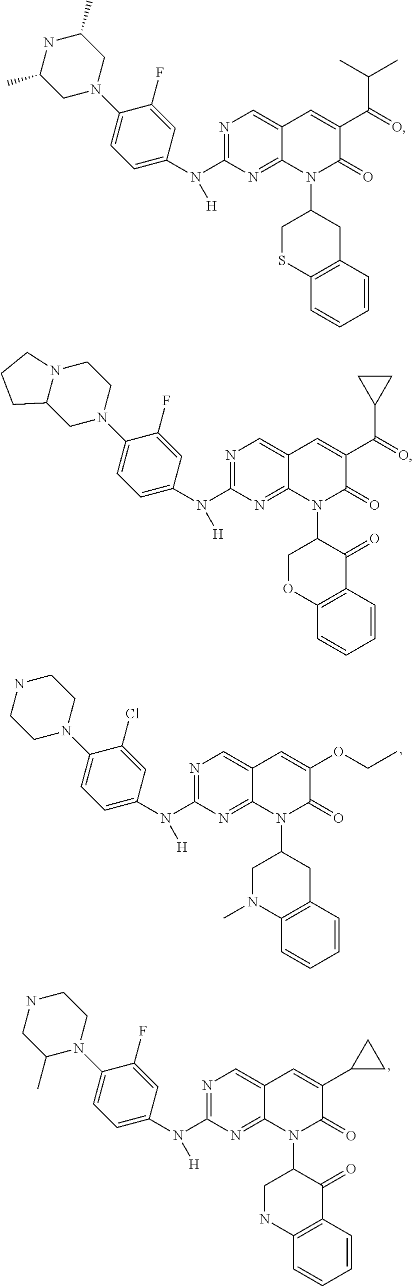 [SMILES: CC(C)C(=O)c1cc2cnc(Nc3ccc(N4C[C@@H](C)N[C@@H](C)C4)c(F)c3)nc2n(C2CSc3ccccc3C2)c1=O ; O=C(c1cc2cnc(Nc3ccc(N4CCN5CCCC5C4)c(F)c3)nc2n(C2COc3ccccc3C2=O)c1=O)C1CC1 ; CCOc1cc2cnc(Nc3ccc(N4CCNCC4)c(Cl)c3)nc2n(C2Cc3ccccc3N(C)C2)c1=O ; CC1CNCCN1c1ccc(Nc2ncc3cc(C4CC4)c(=O)n(C4CNc5ccccc5C4=O)c3n2)cc1F]

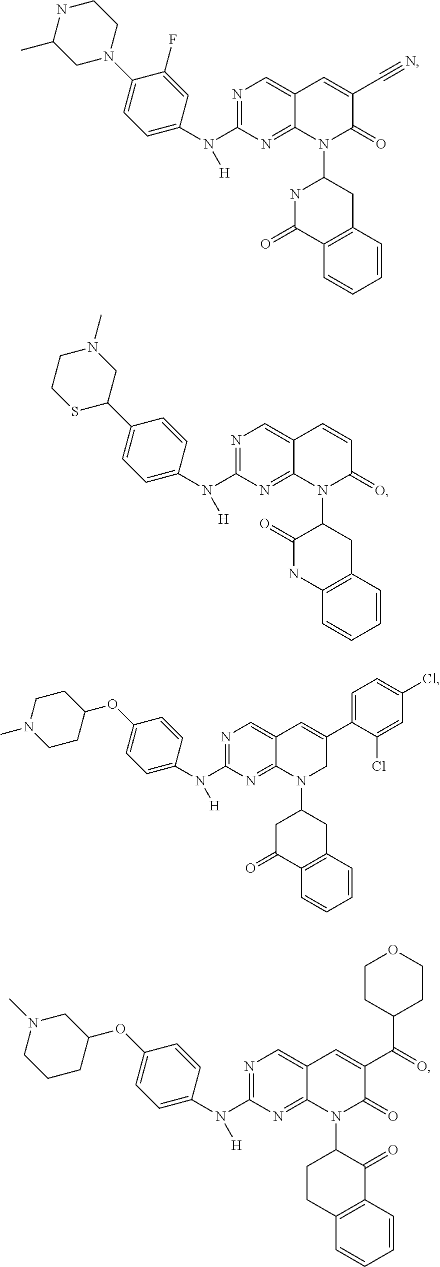

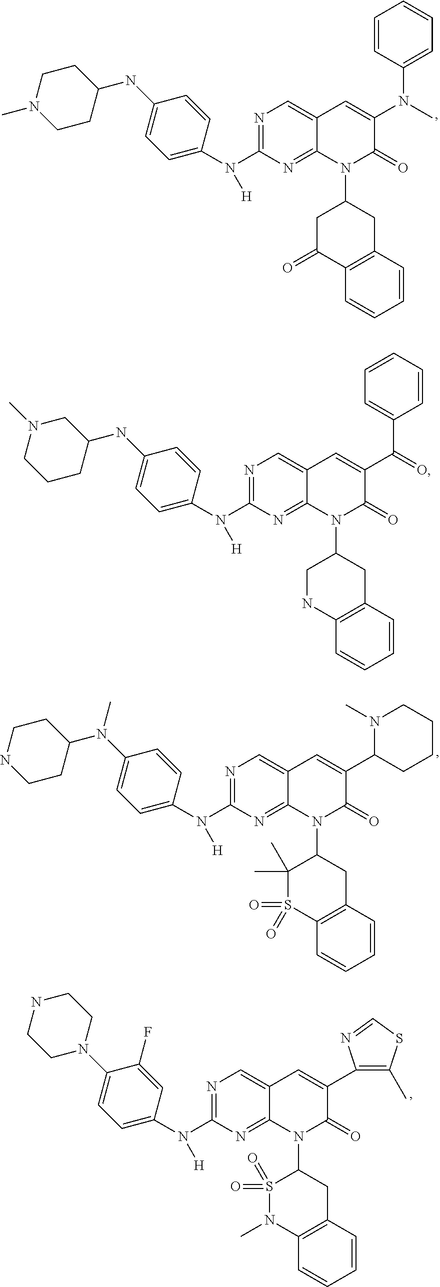

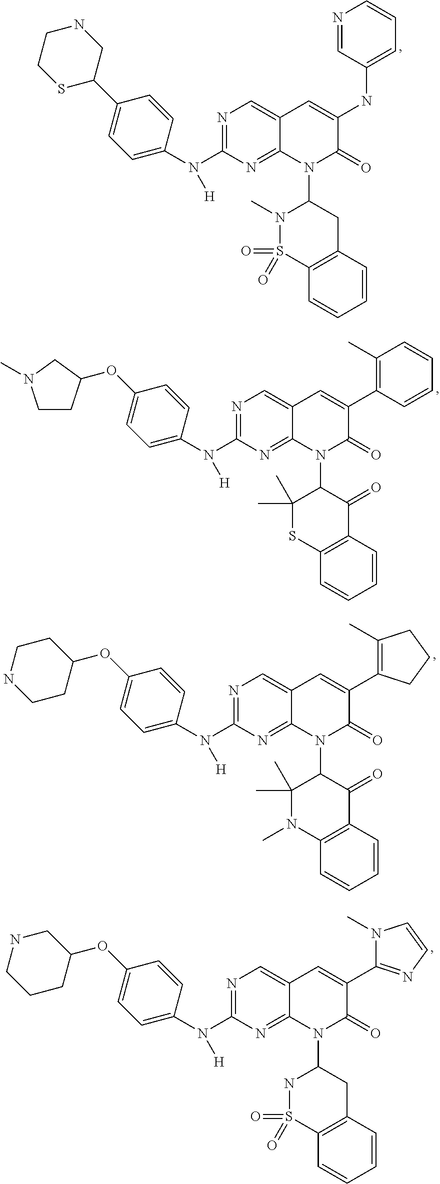

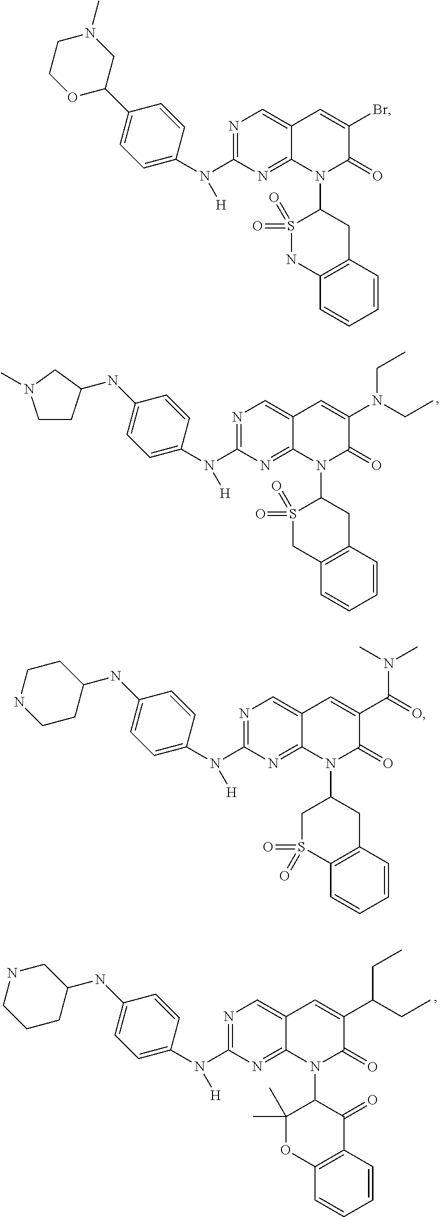

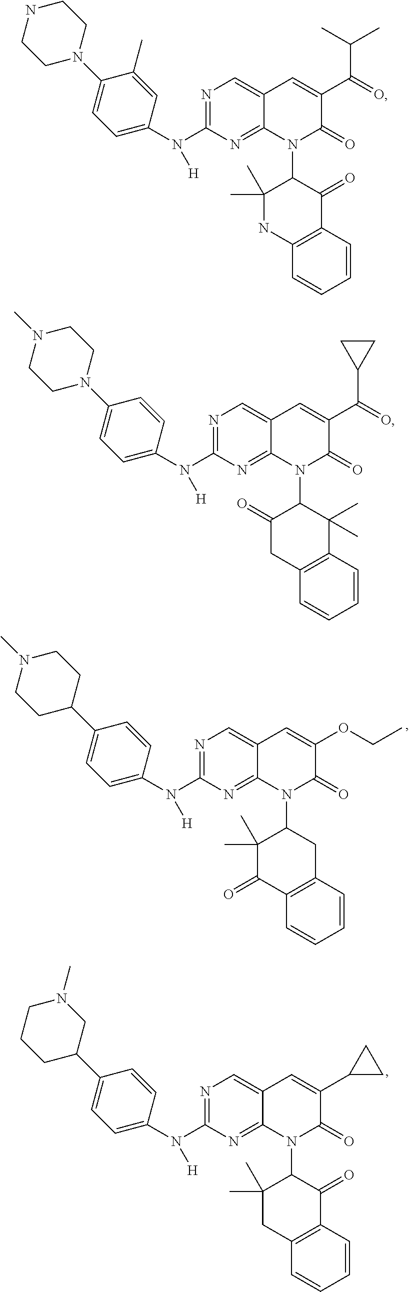

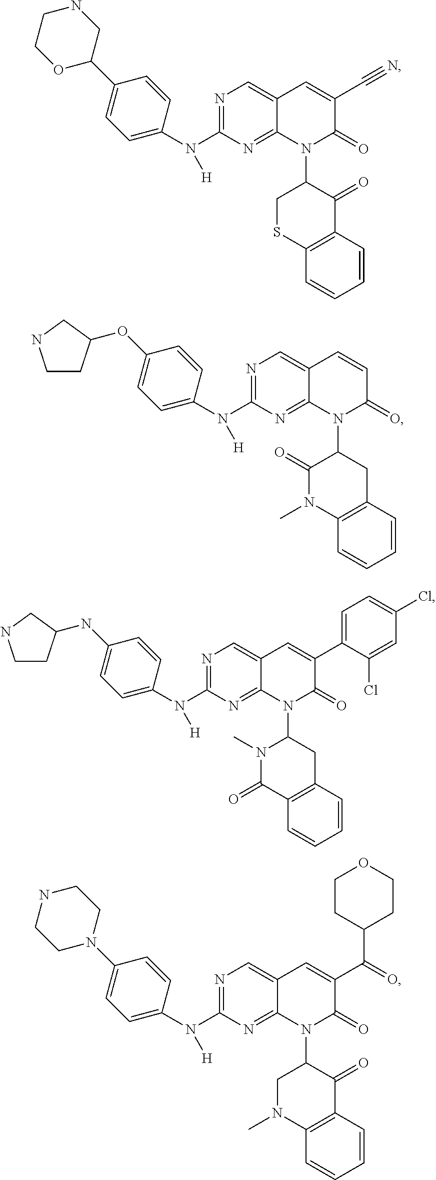

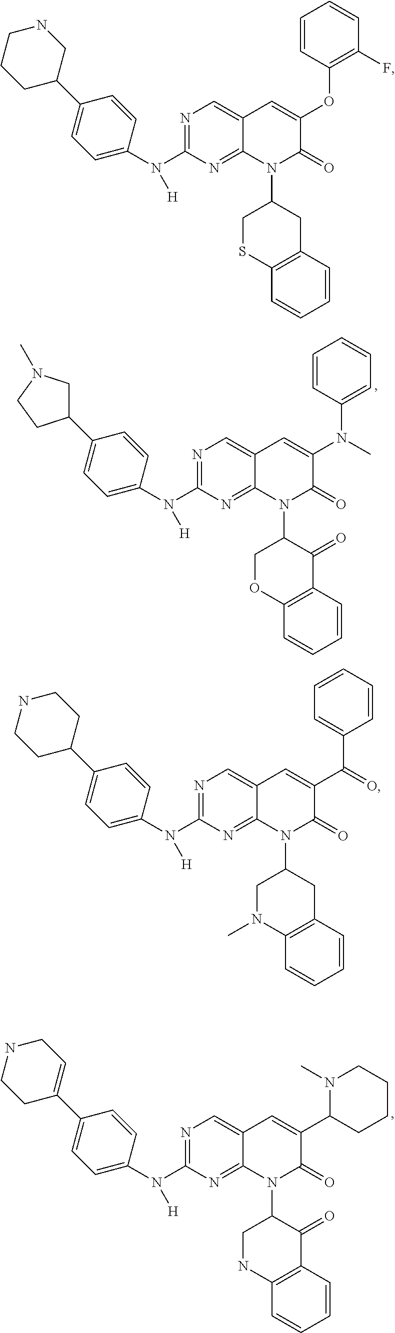

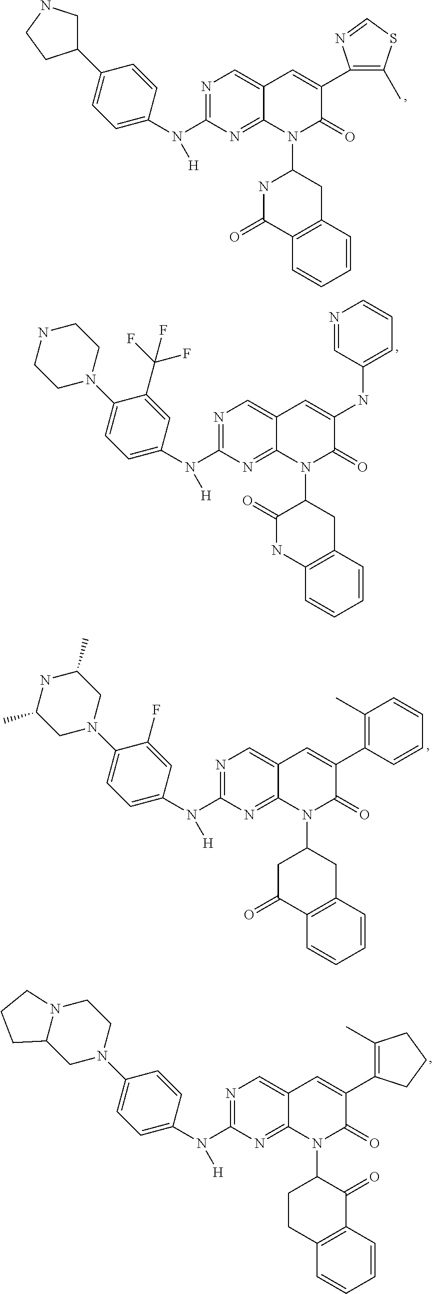

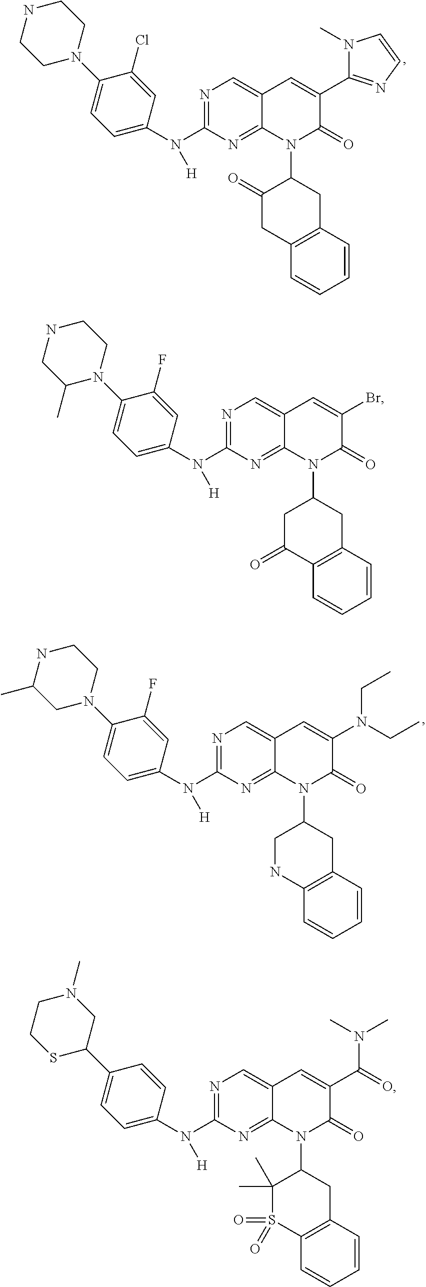 [SMILES: Cn1ccnc1-c1cc2cnc(Nc3ccc(N4CCNCC4)c(Cl)c3)nc2n(C2Cc3ccccc3CC2=O)c1=O ; CC1CNCCN1c1ccc(Nc2ncc3cc(Br)c(=O)n(C4CC(=O)c5ccccc5C4)c3n2)cc1F ; CCN(CC)c1cc2cnc(Nc3ccc(N4CCNC(C)C4)c(F)c3)nc2n(C2CNc3ccccc3C2)c1=O ; CN1CCSC(c2ccc(Nc3ncc4cc(C(=O)N(C)C)c(=O)n(C5Cc6ccccc6S(=O)(=O)C5(C)C)c4n3)cc2)C1]

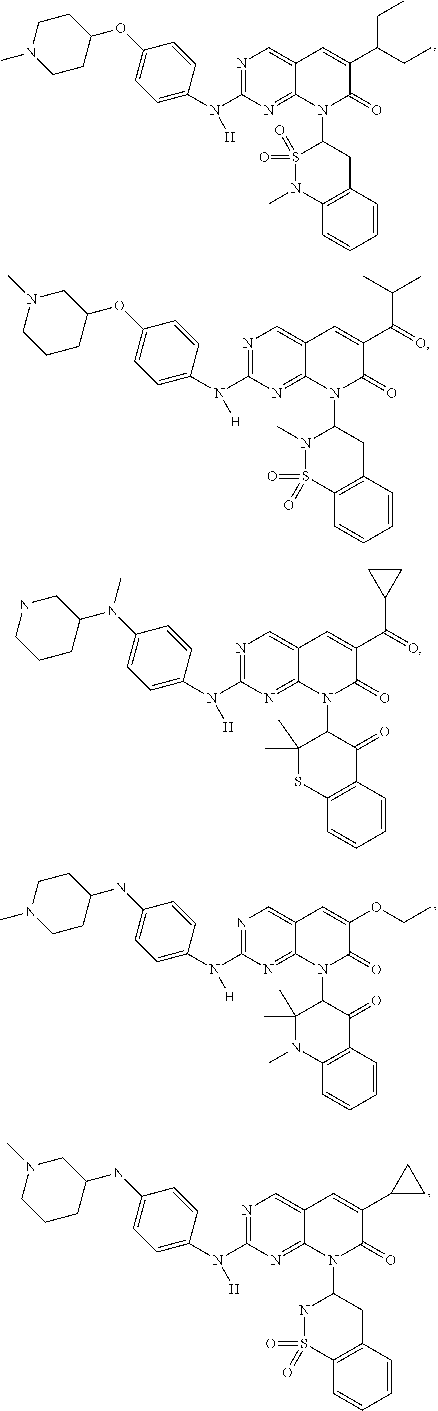

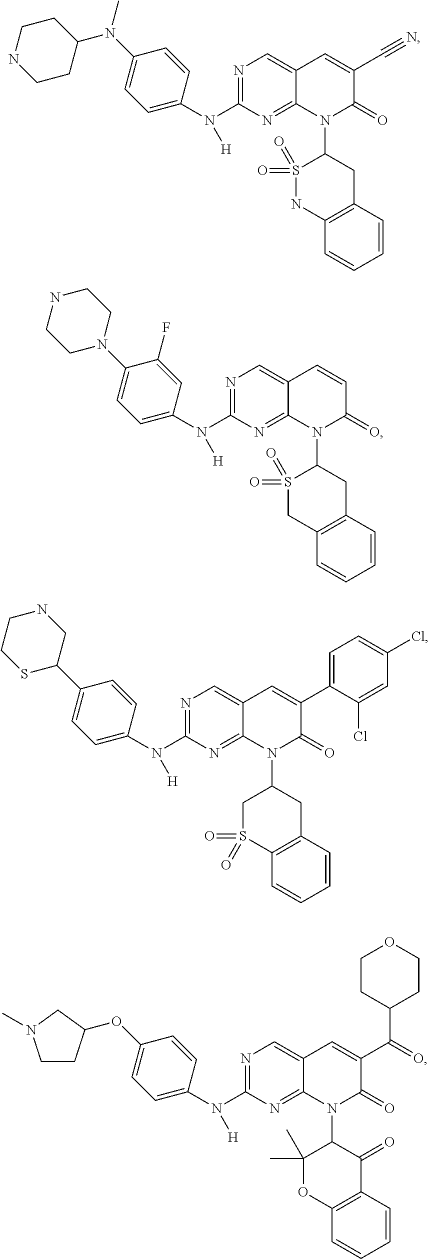 [SMILES: CN(c1ccc(Nc2ncc3cc(C#N)c(=O)n(C4Cc5ccccc5NS4(=O)=O)c3n2)cc1)C1CCNCC1 ; O=c1ccc2cnc(Nc3ccc(N4CCNCC4)c(F)c3)nc2n1C1Cc2ccccc2CS1(=O)=O ; O=c1c(-c2ccc(Cl)cc2Cl)cc2cnc(Nc3ccc(C4CNCCS4)cc3)nc2n1C1Cc2ccccc2S(=O)(=O)C1 ; CN1CCC(Oc2ccc(Nc3ncc4cc(C(=O)C5CCOCC5)c(=O)n(C5C(=O)c6ccccc6OC5(C)C)c4n3)cc2)C1]

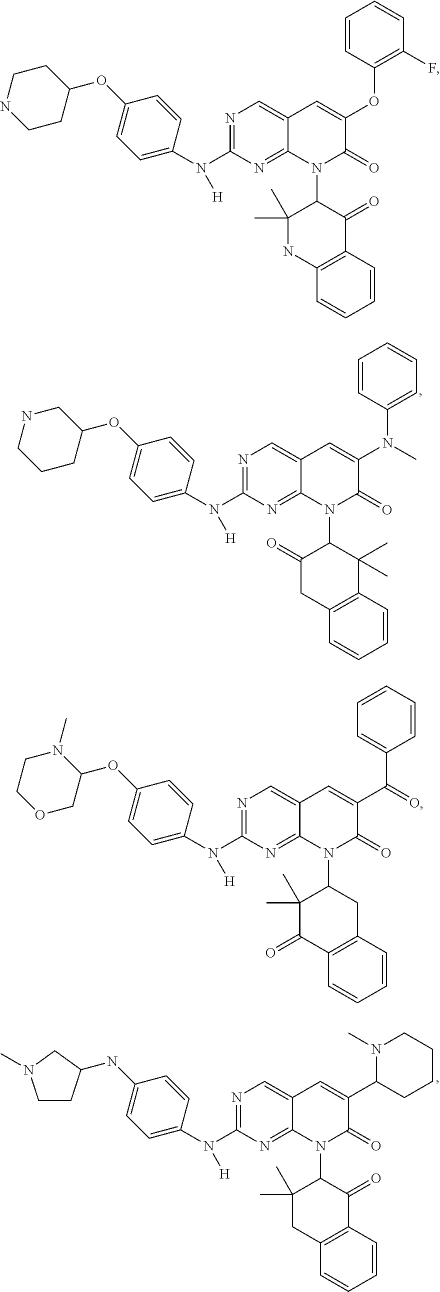

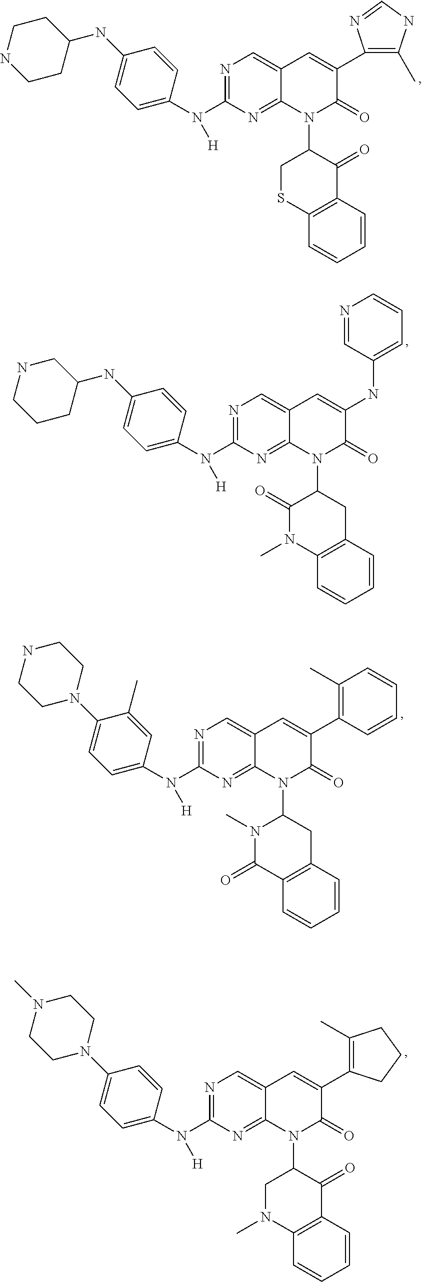

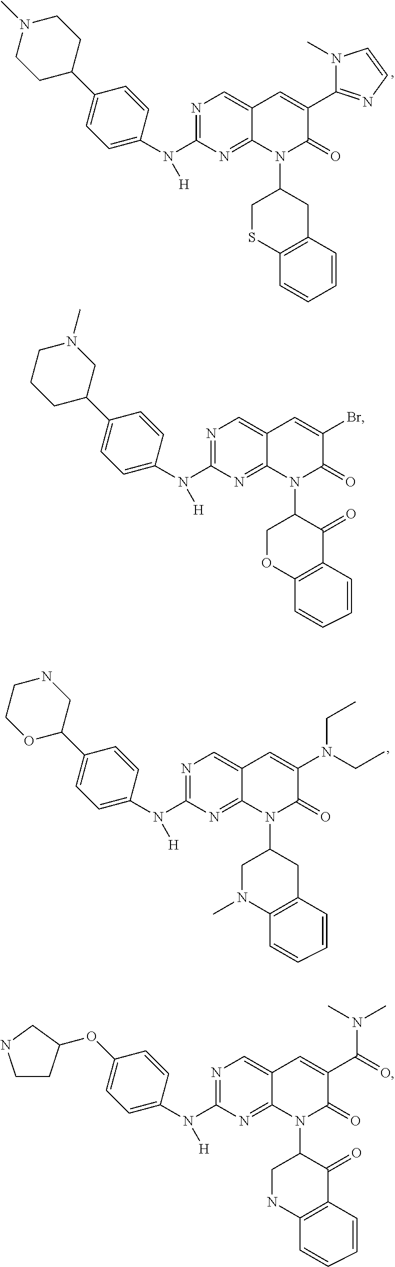

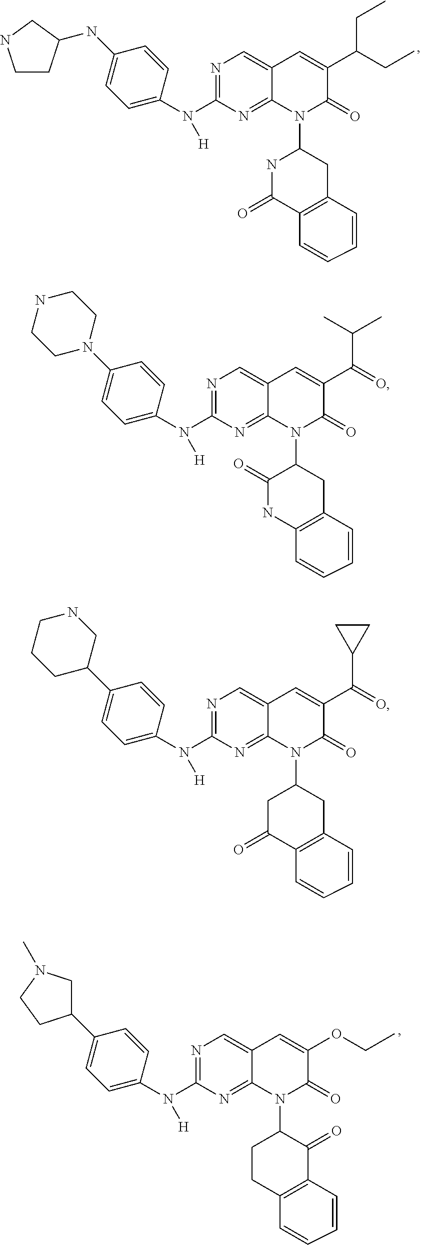 [SMILES: CCC(CC)c1cc2cnc(Nc3ccc(NC4CCNC4)cc3)nc2n(C2Cc3ccccc3C(=O)N2)c1=O ; CC(C)C(=O)c1cc2cnc(Nc3ccc(N4CCNCC4)cc3)nc2n(C2Cc3ccccc3NC2=O)c1=O ; O=C1CC(n2c(=O)c(C(=O)C3CC3)cc3cnc(Nc4ccc(C5CCCNC5)cc4)nc32)Cc2ccccc21 ; CCOc1cc2cnc(Nc3ccc(C4CCN(C)C4)cc3)nc2n(C2CCc3ccccc3C2=O)c1=O]

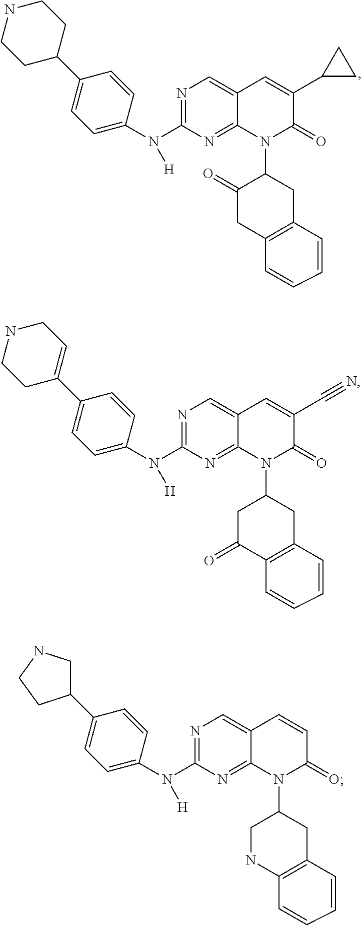 [SMILES: O=C1Cc2ccccc2CC1n1c(=O)c(C2CC2)cc2cnc(Nc3ccc(C4CCNCC4)cc3)nc21 ; N#Cc1cc2cnc(Nc3ccc(C4=CCNCC4)cc3)nc2n(C2CC(=O)c3ccccc3C2)c1=O ; O=c1ccc2cnc(Nc3ccc(C4CCNC4)cc3)nc2n1C1CNc2ccccc2C1]

or a pharmaceutically acceptable salt, solvate or N-oxide thereof.

In another embodiment are PAK inhibitors suitable for treating cancer having the structure of Formula IV or a pharmaceutically acceptable salt, solvate or N-oxide thereof wherein:

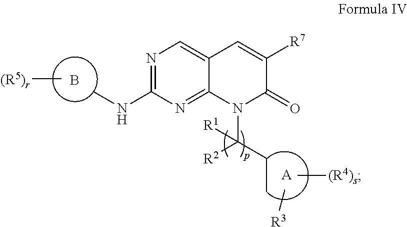

wherein:
R¹ and R² are each independently H or substituted or unsubstituted alkyl; or R¹ and R² together with the carbon to which they are attached form a C₃-C₆ cycloalkyl ring;
p is 1, 2 or 3;
ring A is aryl or heteroaryl;
R³ is S(═O)R⁹ or —S(═O)₂R⁹;
each R⁴ is independently halogen, —CN, —NO₂, —OH, —OCF₃, —OCF₂H, —OCH₂F, —CF₃, —SR⁸, —S(═O)R⁹, —S(═O)₂R⁹, —NR¹⁰S(═O)₂R⁹, —S(═O)₂N(R¹⁰)₂, —OR¹⁰, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰(═O)OR¹⁰,
   —NR¹⁰C(═O)N(R¹⁰)₂, substituted or unsubstituted alkyl, substituted or unsubstituted heteroalkyl, substituted or unsubstituted cycloalkyl or substituted or unsubstituted heterocycloalkyl;
R⁸ is H, substituted or unsubstituted alkyl, substituted or unsubstituted cycloalkyl, substituted or unsubstituted heterocycloalkyl, substituted or unsubstituted aryl or substituted or unsubstituted heteroaryl;
R⁹ is substituted or unsubstituted alkyl, substituted or unsubstituted cycloalkyl, substituted or unsubstituted heterocycloalkyl, substituted or unsubstituted aryl or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl or substituted or unsubstituted heteroaryl, or two R¹⁰ together with the atoms to which they are attached form a heterocycle;
s is 0-4;
ring B is aryl or heteroaryl;
each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, substituted or unsubstituted alkyl, substituted or unsubstituted heteroalkyl, substituted or unsubstituted cycloalkyl or substituted or unsubstituted heterocycloalkyl;
r is 0-8;
R⁷ is H, halogen, —CN, substituted or unsubstituted alkyl, —C(═O)N(R¹⁰)₂, —CO₂R¹⁰, —OR¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In one embodiment, is a compound of Formula IV wherein ring A is a 5-10-membered heteroaryl ring comprising 0-4 N atoms, 0-2 O atoms, 0-2 S atoms, or any combination thereof; wherein at least one N, S, or O atom is present.

In another embodiment, is a compound of wherein ring A is pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, or pyrazine. In one embodiment, ring A is pyridine. In another embodiment, ring A is imidazole. In a further embodiment, ring A is oxazole. In yet another embodiment, ring A is pyrazole. In yet another embodiment, ring A is furan. In yet another embodiment, ring A is pyrimidine. In yet another embodiment, ring A is pyrazine. In a further embodiment is a compound of Formula IV wherein ring A is furan.

In a further embodiment, is a compound of Formula IV wherein R³ is S(═O)R⁹. In one embodiment is a compound of Formula IV wherein R³ is (R)—S(═O)R⁹. In another embodiment is a compound of Formula IV wherein R³ is (S)—S(═O)R⁹. In yet a further embodiment, R³ is S(═O)₂R⁹. In one embodiment is a compound of Formula IV wherein R⁹ is substituted or unsubstituted alkyl. In another embodiment is a compound of Formula IV wherein R⁹ is selected from methyl, ethyl, n-propyl, iso-propyl, n-butyl, iso-propyl, or tert-butyl. In a further embodiment is a compound of Formula IV wherein R⁹ is methyl. In one embodiment, the alkyl is substituted with a group selected from —CN, —NO₂, —OH, —SH, S—C₁-C₆alkyl, —S(═O)H, —S(═O)C₁-C₆alkyl, —S(═O)₂H, —S(═O)₂C₁-C₆alkyl, NR¹⁰S(═O)₂H, —S(═O)₂N(R¹⁰)₂, —C(═O)H, —C(═O)—C₁-C₆alkyl, —OC(═O)H, —OC(═O)C1-C6alkyl, —CO₂R¹⁰, —N(R¹⁰)₂, —C(═O)N(R¹⁰)₂, —NR¹⁰C(O)R¹⁰, —NR¹⁰C(═O)OR¹⁰, —NR¹⁰C(═O)N(R¹⁰)₂, —O—C₁-C₆alkyl. In another embodiment, R⁹ is alkyl substituted with NH₂. In another embodiment, R⁹ is alkyl substituted with OCH₃. In another embodiment is a compound of Formula IV wherein R⁹ is substituted or unsubstituted cycloalkyl. In a further embodiment, the substituted or unsubstituted alkyl is cyclopropyl, cyclobutyl, cyclopentyl, cyclohexyl or cycloheptyl. In a further embodiment, R⁹ is cyclopentyl. In yet a further embodiment, R⁹ is a substituted or unsubstituted heterocycloalkyl selected from piperidine, piperazine, pyrrolidine or morpholine. In another embodiment R⁹ is piperidine. In a further embodiment is a compound of Formula IV wherein R⁹ is substituted or unsubstituted aryl. In one embodiment, R⁹ is phenyl. In a further embodiment, phenyl is substituted with a halogen. In yet another embodiment, the halogen is F, Cl, Br, or I. In another embodiment, the halogen is Cl. In one embodiment the aryl is naphthalene.

Also disclosed herein is a compound of Formula IV wherein R⁹ is substituted or unsubstituted heteroaryl. In one embodiment, the heteroaryl is selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, and pyrazine. In one embodiment, the heteroaryl is furan. In another embodiment the heteroaryl is pyridine.

In yet another embodiment is a compound of Formula IV wherein each R⁴ is selected from halogen, —CN, —NO₂, —OH, —OCF₃, —OCF₂H, —OCH₂F, —CF₃, —SR⁸, —S(═O)R⁹, —S(═O)₂R⁹, —NR¹⁰S(═O)₂R⁹, —S(═O)₂N(R¹⁰)₂, —OR¹⁰, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂. In another embodiment is a compound of Formula IV wherein each R⁴ is selected from substituted or unsubstituted alkyl, substituted or unsubstituted heteroalkyl, substituted or unsubstituted cycloalkyl or substituted or unsubstituted heterocycloalkyl. In one embodiment, R⁴ is selected from F, Cl, Br, or I. In another embodiment, R⁴ is OH. In a further embodiment, R⁴ is OCH₃. In yet another embodiment, R⁴ is OCF₃. In one embodiment is a compound of Formula IV wherein s is 0. In another embodiment, s is 1. In a further embodiment, s is 2, 3, or 4.

In one embodiment is a compound of Formula IV wherein R¹ and R² are each independently H. In another embodiment, is a compound of Formula IV wherein p is 1. In a further embodiment, is a compound of Formula IV wherein p is 2. In another embodiment is a compound of Formula IV wherein R¹ is methyl and R² is H. In yet a further embodiment is a compound of Formula IV wherein R¹ and R² are each substituted alkyl. In a further embodiment each R¹ and R² are alkyl substituted with a halogen. In another embodiment, each R¹ and R² are alkyl substituted with a OH. In a further embodiment is a compound of Formula IV wherein R¹ and R² together with the carbon to which they are attached form a C₃-C₆cycloalkyl ring. In a further embodiment, is a compound of Formula IV wherein R¹ and R² together with the carbon to which they are attached form a cyclopentyl group. In one embodiment is a compound of Formula IV wherein p is 1. In another embodiment, p is 2. In a further embodiment, p is 3.

In yet another embodiment, is a compound of Formula IV, wherein ring B is an aryl ring. In another embodiment, ring B is a substituted or unsubstituted phenyl. In a further embodiment, ring B is a substituted or unsubstituted naphthalene. In a further embodiment, is a compound of Formula IV, wherein ring B is a heteroaryl ring selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, and pyrazine.

In yet a further embodiment, is a compound of Formula IV, wherein R⁵ is a C₃-C₆ cycloalkyl ring; or a 3-6-membered heterocycloalkyl ring comprising 1-3 N atoms, an O atom, a S atom; or any combination thereof, and wherein R⁵ is further substituted by halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, substituted or unsubstituted alkyl, substituted or unsubstituted alkoxy, substituted or unsubstituted heteroalkyl, substituted or unsubstituted cycloalkyl or substituted or unsubstituted heterocycloalkyl.

In another embodiment, is a compound of Formula IV wherein R⁵ is a C₃-C₆cycloalkyl ring. In another embodiment, R⁵ is cyclopropyl. In another embodiment, R⁵ is cyclopentyl. In another embodiment, R⁵ is cyclohexyl.

In another embodiment, is a compound of Formula IV wherein R⁵ is a substituted or unsubstituted heterocycloalkyl ring. In another embodiment, R⁵ is a substituted or unsubstituted piperazine. In another embodiment, R⁵ is an unsubstituted piperazine. In another embodiment, R⁵ is N-methylpiperazine. In another embodiment, R⁵ is a substituted or unsubstituted piperidine. In another embodiment, R⁵ is an unsubstituted piperidine.

In another embodiment, R⁵ is OH or CN. In a further embodiment, R⁵ is OCF₃, or CF₃.

In another embodiment is a compound of Formula IV wherein r is 0. In another embodiment, r is 1. In a further embodiment, r is 2. In another embodiment is a compound of Formula IV wherein r is 1; and R⁵ is substituted or unsubstituted piperazine.

In one embodiment is a compound of Formula IV wherein

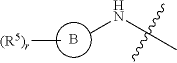

is selected from:

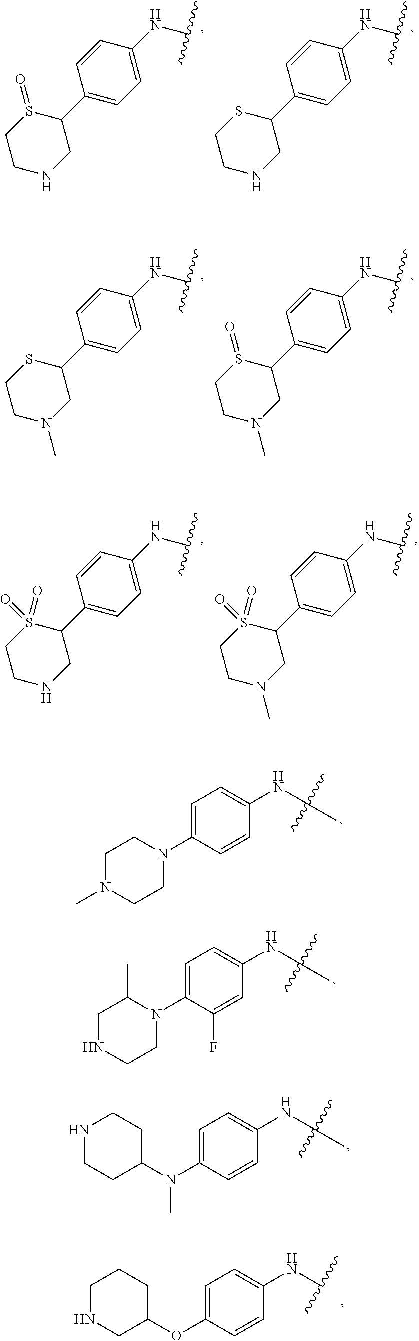

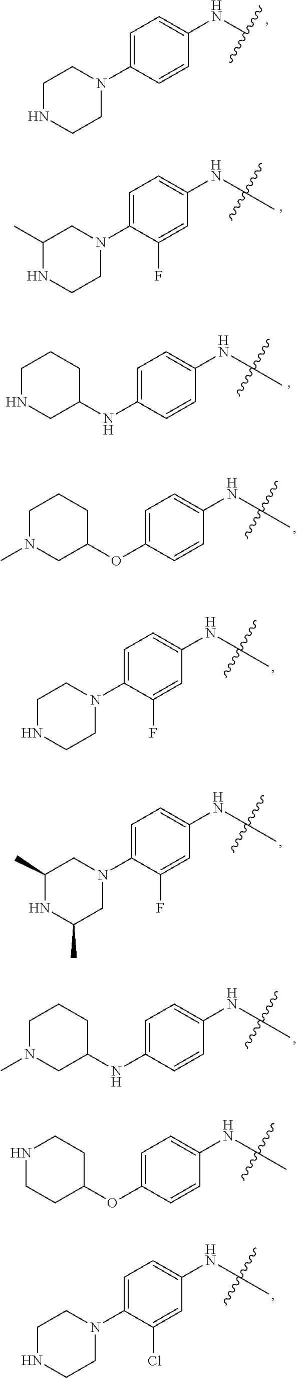

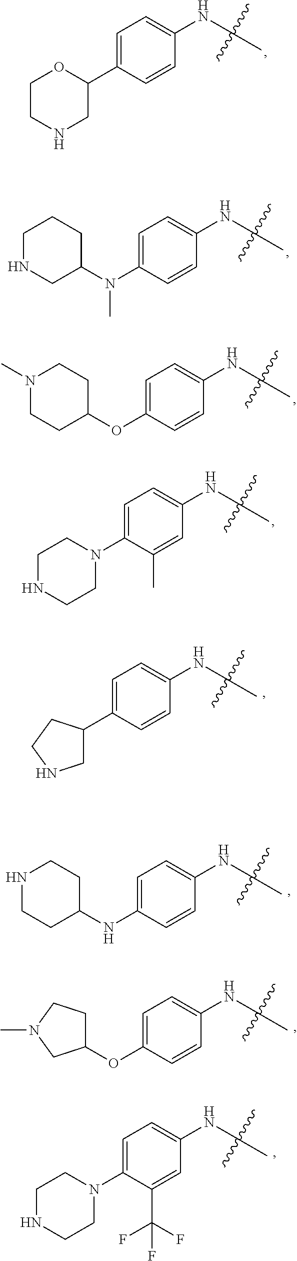

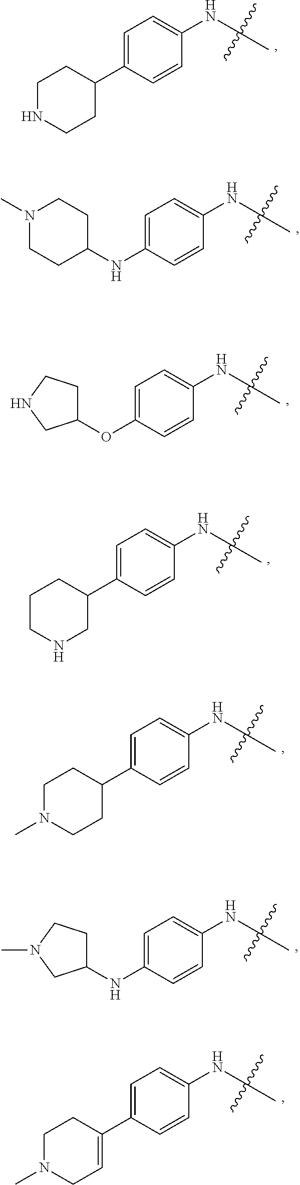

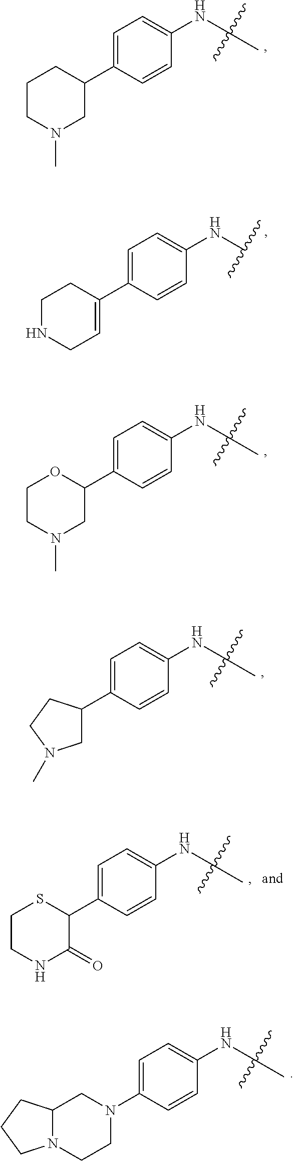 [SMILES: *Nc1ccc(C2CCCN(C)C2)cc1 ; *Nc1ccc(C2=CCNCC2)cc1 ; *Nc1ccc(C2CN(C)CCO2)cc1 ; *Nc1ccc(C2CCN(C)C2)cc1 ; *Nc1ccc(C2SCCNC2=O)cc1 ; *Nc1ccc(N2CCN3CCCC3C2)cc1]

In another embodiment is a compound of Formula IV, wherein

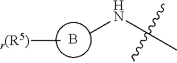

is selected from:

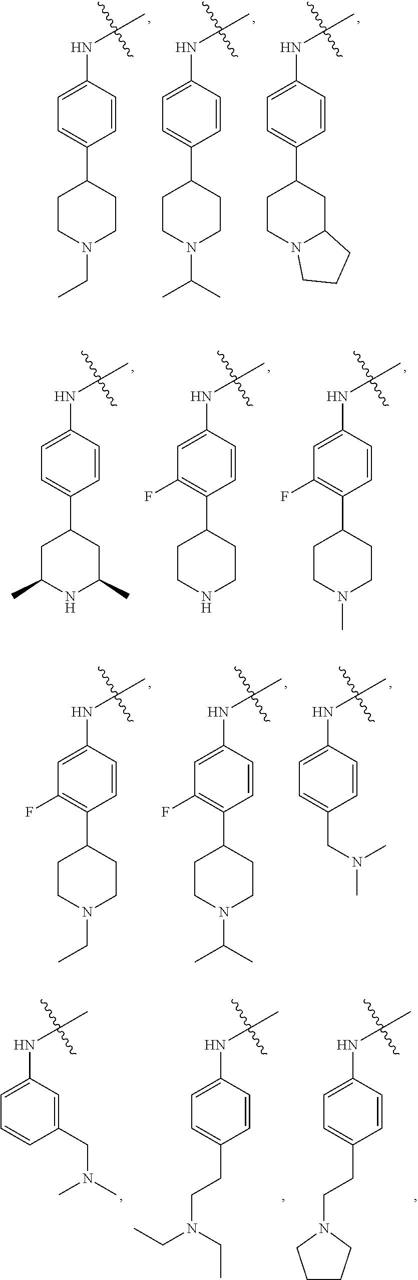

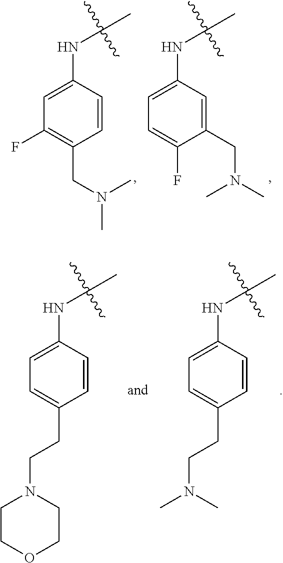

In a further embodiment is a compound of Formula IV, wherein

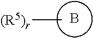

is:

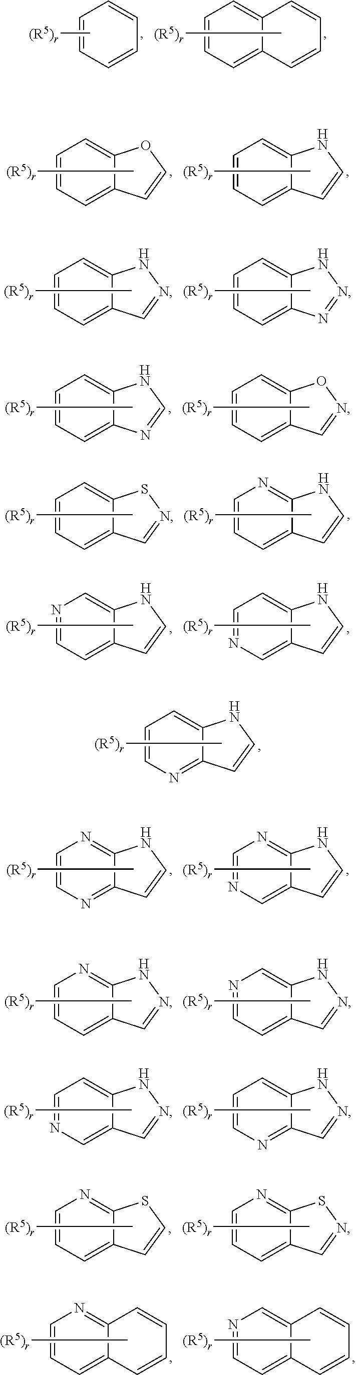

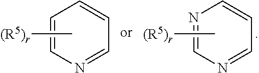

In one embodiment, is a compound of Formula IV, wherein R⁵ is halogen, —CN, —OH, substituted or unsubstituted alkyl, —OR¹⁰, —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═)N(R¹⁰)₂, or substituted or unsubstituted heterocycloalkyl. In one embodiment, R⁵ is selected from F, Cl, Br, or I. In another embodiment R⁵ is F.

In another embodiment, is a compound of Formula IV, wherein at least one R⁵ is —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In one embodiment, is a compound of Formula IV, wherein at least one R⁵ is —N(R¹⁰)₂, or substituted or unsubstituted heterocycloalkyl. In yet another embodiment, is a compound of Formula IV wherein at least one of R⁵ is a substituted or unsubstituted piperazine, substituted or unsubstituted piperidine, substituted or unsubstituted pyrrolidine or substituted or unsubstituted morpholine. In a further embodiment, is a compound of Formula IV, wherein at least one R⁵ is —OR¹⁰. In one embodiment is a compound of Formula IV, wherein at least one R⁵ is —OR¹⁰ and R¹⁰ is H. In another embodiment, R¹⁰ is alkyl selected from methyl, ethyl, n-propyl, iso-propyl, n-butyl, iso-butyl, and tert-butyl.

In one embodiment is a compound of Formula IV wherein ring B is substituted with —N(R¹⁰)₂, wherein R¹⁰ is each independently selected from H and a substituted or unsubstituted heterocycloalkyl. In another embodiment is a compound of Formula IV wherein ring B is substituted with —NHR¹⁰ wherein R¹⁰ is a substituted or unsubstituted piperazine, substituted or unsubstituted piperidine, substituted or unsubstituted pyrrolidine or substituted or unsubstituted morpholine. In a further embodiment is a compound of Formula IV wherein ring B is substituted with —N(CH₃)R¹⁰ wherein R¹⁰ is a substituted or unsubstituted piperazine, substituted or unsubstituted piperidine, substituted or unsubstituted pyrrolidine or substituted or unsubstituted morpholine.

Also presented herein is a compound of Formula IV wherein ring B is substituted with —OR¹⁰ wherein R¹⁰ is a substituted or unsubstituted heterocycloalkyl. In another embodiment is a compound of Formula IV wherein ring B is substituted with —OR¹⁰ wherein R¹⁰ is a substituted or unsubstituted piperazine, substituted or unsubstituted piperidine, substituted or unsubstituted pyrrolidine or substituted or unsubstituted morpholine. In yet another embodiment is a compound of Formula IV wherein ring B is substituted with at least one CF₃.

In yet another embodiment, ring B is substituted with at least two R⁵. In another embodiment, ring B is substituted with halogen and a substituted or unsubstituted heterocycloalkyl. In another embodiment, ring B is substituted with at least one F, Cl, Br, or I and a substituted or unsubstituted piperazine, substituted or unsubstituted piperidine, substituted or unsubstituted pyrrolidine, or substituted or unsubstituted morpholine.

In yet a further embodiment, is a compound of Formula IV, wherein R⁴ is independently halogen, —CN, —OH, —OCF₃, —OCHF₂, —OCF₂H, —CF₃, —SR⁸, —S(═O)R⁹, —S(═O)₂R⁹, substituted or unsubstituted alkyl, or substituted or unsubstituted alkoxy. In one embodiment, R⁴ is halogen. In another embodiment, R⁴ is selected from F, Cl, Br, and I. In a further embodiment, R⁴ is F.

In another embodiment are PAK inhibitors suitable for treating cancer having the structure of Formula IVa or a pharmaceutically acceptable salt, solvate or N-oxide thereof wherein:

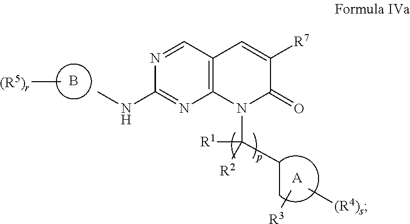

wherein:
R¹ and R² are each independently H or substituted or unsubstituted alkyl; or R¹ and R² together with the carbon to which they are attached form a C₃-C₆ cycloalkyl ring;
p is 1, 2 or 3;
ring A is aryl or heteroaryl;
R³ is —S(═O)R⁹, (R) or (S)—S(═O)R⁹ or —S(═O)₂R⁹;
each R⁴ is independently halogen, —CN, —NO₂, —OH, —OCF₃, —OCF₂H, —CF₃, —SR⁸, —S(═O)R⁹, —S(═O)₂R⁹, —NR¹⁰S(═O)₂R⁹, —S(═O)₂N(R¹⁰)₂, —OR¹⁰, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted heteroalkyl, substituted or unsubstituted cycloalkyl or substituted or unsubstituted heterocycloalkyl;
R⁸ is H, substituted or unsubstituted alkyl, substituted or unsubstituted cycloalkyl, substituted or unsubstituted heterocycloalkyl, substituted or unsubstituted aryl or substituted or unsubstituted heteroaryl;
R⁹ is substituted or unsubstituted alkyl, substituted or unsubstituted cycloalkyl, substituted or unsubstituted heterocycloalkyl, substituted or unsubstituted aryl or substituted or unsubstituted heteroaryl
each R¹⁰ is independently H, substituted or unsubstituted alkyl, substituted or unsubstituted cycloalkyl, substituted or unsubstituted heterocycloalkyl, substituted or unsubstituted aryl or substituted or unsubstituted heteroaryl, or two R¹⁰ together with the atoms to which they are attached form a heterocycle;
s is 0-4;
ring B is aryl or heteroaryl;
each R⁵ is independently halogen, —CN, —NO₂, —OH, —SR⁸, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═)OR¹⁰, —NR¹⁰C(═O)N(R¹⁰)₂, —OR¹⁰, substituted or unsubstituted alkyl, substituted or unsubstituted heteroalkyl, substituted or unsubstituted cycloalkyl or substituted or unsubstituted heterocycloalkyl;
r is 0-8;
R⁷ is H, halogen, —CN, substituted or unsubstituted alkyl, —C(═O)N(R¹⁰)₂, —CO₂R¹⁰, —OR¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl;
provided that the compound of Formula IVa is not 2-(4-(4-methylpiperazin-1-yl)phenylamino)-8-(2-(methylsulfonyl)benzyl)pyrido[2,3-d]pyrimidin-7(8H)-one, 8-(2-fluoro-6-(methylsulfinyl)benzyl)-2-(4-(4-methylpiperazin-1-yl)phenylamino)pyrido[2,3-d]pyrimidin-7(8H)-one, or 8-(2-fluoro-6-(methylsulfinyl)benzyl)-2-(4-(4-methylpiperazin-1-yl)phenylamino)pyrido[2,3-d]pyrimidin-7(8H)-one.

In one embodiment, is a compound of Formula IV or IVa, wherein p is 1; and R¹ and R² are each H. In another embodiment, is a compound of Formula IV or IVa, wherein R⁷ is H. In yet another embodiment is a compound of Formula IV or IVa wherein R⁷ is —CN. In a further embodiment, is a compound of Formula IV or IVa wherein R⁷ is substituted or unsubstituted cyclopropyl, substituted or unsubstituted cyclobutyl, substituted or unsubstituted cyclopentyl or substituted or unsubstituted cyclohexyl.

In yet a further embodiment, is a compound of Formula IV or IVa wherein R⁷ is substituted or unsubstituted morpholino, substituted or unsubstituted piperazinyl, substituted or unsubstituted piperidinyl.

In one embodiment, is a compound of Formula IV or IVa wherein R⁷ is substituted or unsubstituted acyl.

In another embodiment, is a compound of Formula IV or IVa, wherein R⁷ is substituted or unsubstituted aryl or substituted or unsubstituted heteroaryl.

In yet another embodiment, is a compound of Formula IV or IVa wherein substituted or unsubstituted aryl or substituted or unsubstituted heteroaryl has the structure:

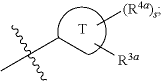

wherein:
ring T is aryl, or heteroaryl;
R^{3a} is H or R^{4a};
each R^{4a} is independently halogen, —CN, —NO₂, —OH, —OCF₃, —OCF₂H, —OCH₂F, —CF₃, —SR⁸, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, substituted or unsubstituted alkyl, substituted or unsubstituted heteroalkyl, substituted or unsubstituted cycloalkyl or substituted or unsubstituted heterocycloalkyl;
R⁸ is H, substituted or unsubstituted alkyl, substituted or unsubstituted cycloalkyl, substituted or unsubstituted heterocycloalkyl, substituted or unsubstituted aryl or substituted or unsubstituted heteroaryl;
R⁹ is substituted or unsubstituted alkyl, substituted or unsubstituted cycloalkyl, substituted or unsubstituted aryl or substituted or unsubstituted heteroaryl, substituted or unsubstituted heterocycloalkyl;
each R¹⁰ is independently H, substituted or unsubstituted alkyl, substituted or unsubstituted cycloalkyl, substituted or unsubstituted aryl or substituted or unsubstituted heteroaryl, substituted or unsubstituted heterocycloalkyl, or two R¹⁰ together with the atoms to which they are attached form a heterocycle; and
s is 0-4.

In yet another embodiment is a compound of Formula IV or IVa wherein R⁷ is selected from:

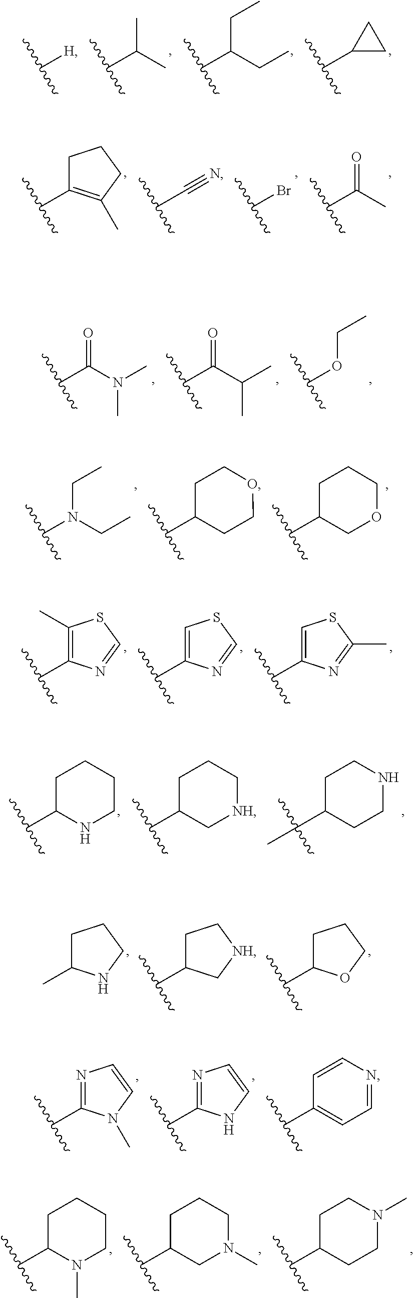

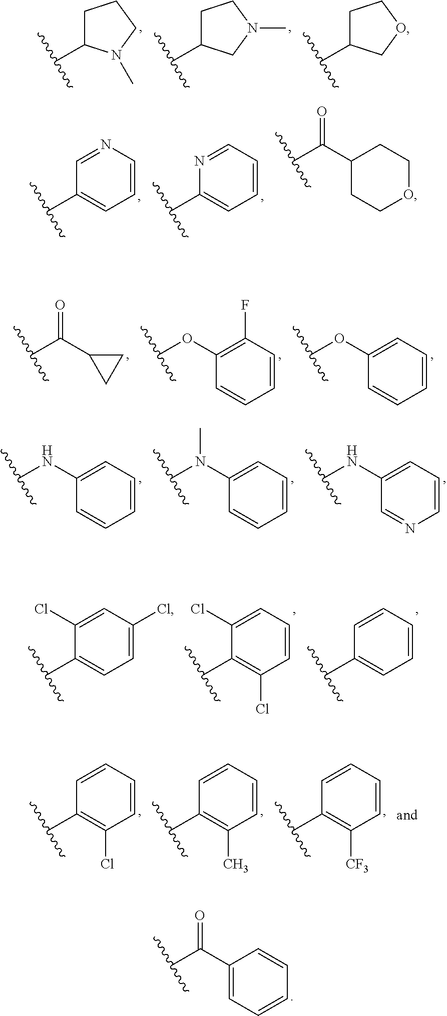

In a further aspect is a compound having the structure:

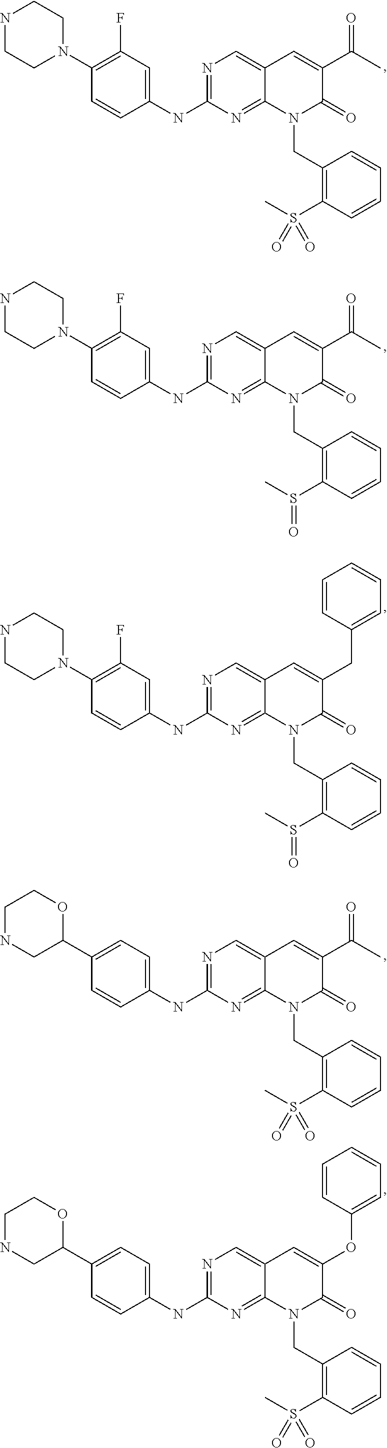

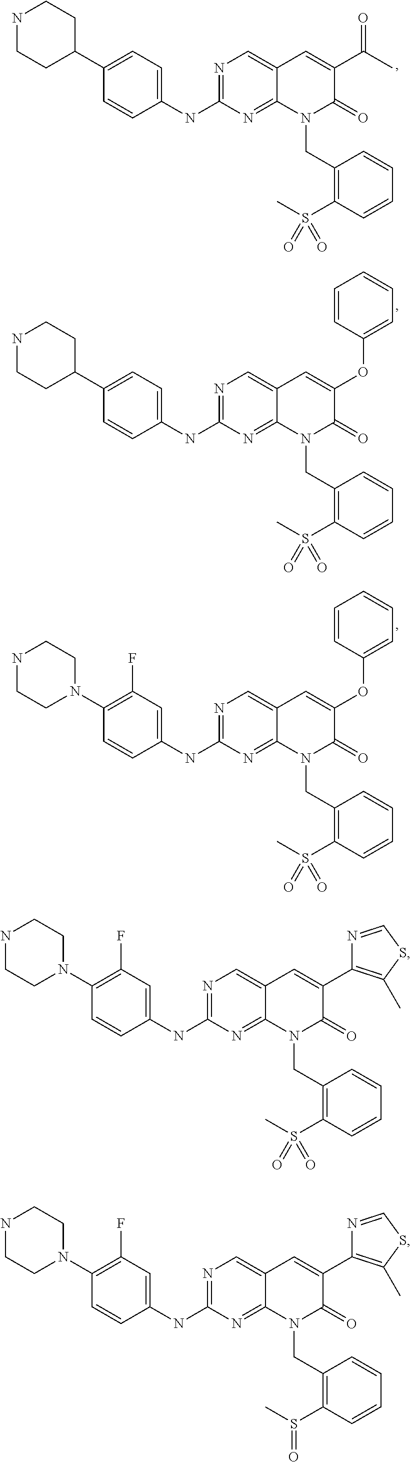

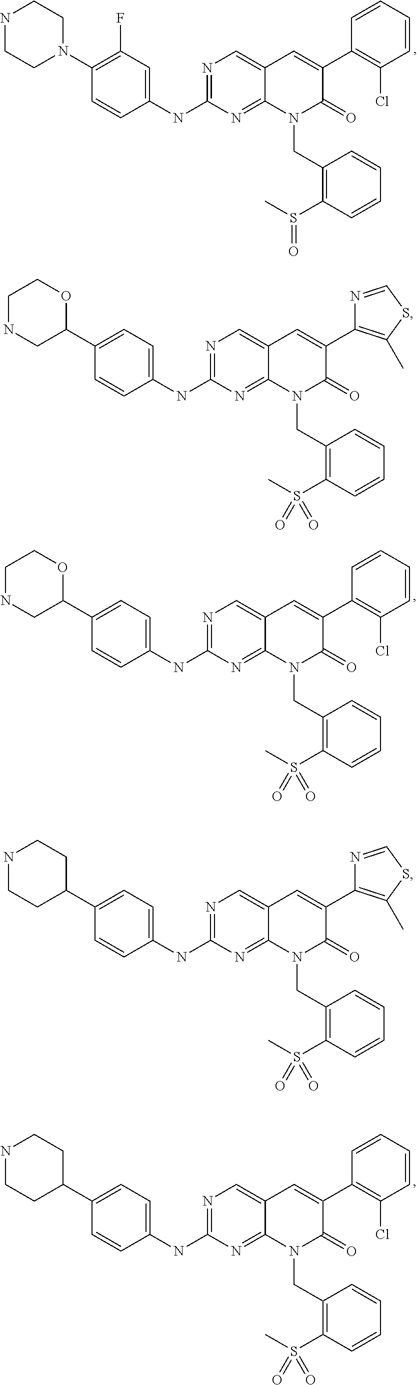 [SMILES: CS(=O)c1ccccc1Cn1c(=O)c(-c2ccccc2Cl)cc2cnc(Nc3ccc(N4CCNCC4)c(F)c3)nc21 ; Cc1scnc1-c1cc2cnc(Nc3ccc(C4CNCCO4)cc3)nc2n(Cc2ccccc2S(C)(=O)=O)c1=O ; CS(=O)(=O)c1ccccc1Cn1c(=O)c(-c2ccccc2Cl)cc2cnc(Nc3ccc(C4CNCCO4)cc3)nc21 ; Cc1scnc1-c1cc2cnc(Nc3ccc(C4CCNCC4)cc3)nc2n(Cc2ccccc2S(C)(=O)=O)c1=O ; CS(=O)(=O)c1ccccc1Cn1c(=O)c(-c2ccccc2Cl)cc2cnc(Nc3ccc(C4CCNCC4)cc3)nc21]

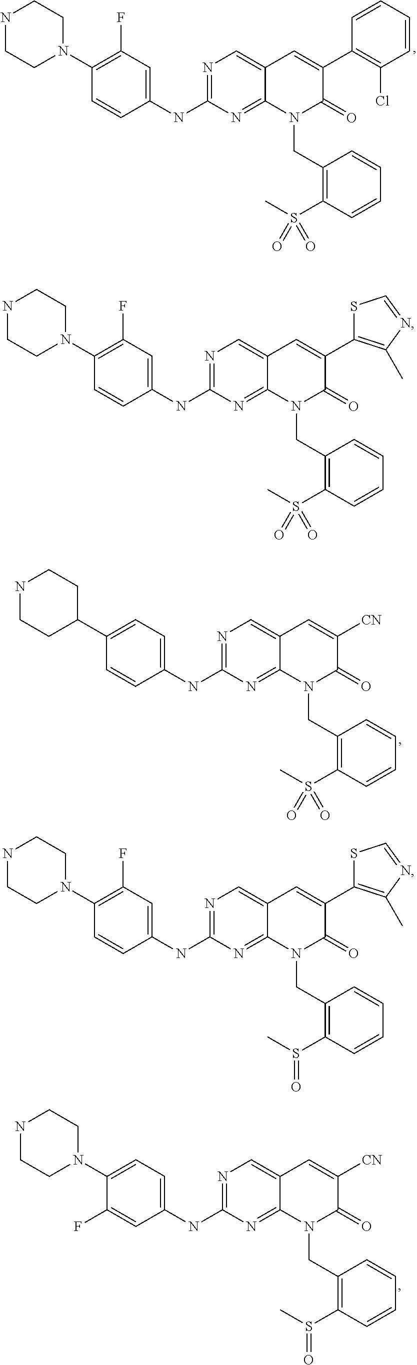 [SMILES: CS(=O)(=O)c1ccccc1Cn1c(=O)c(-c2ccccc2Cl)cc2cnc(Nc3ccc(N4CCNCC4)c(F)c3)nc21 ; Cc1ncsc1-c1cc2cnc(Nc3ccc(N4CCNCC4)c(F)c3)nc2n(Cc2ccccc2S(C)(=O)=O)c1=O ; CS(=O)(=O)c1ccccc1Cn1c(=O)c(C#N)cc2cnc(Nc3ccc(C4CCNCC4)cc3)nc21 ; Cc1ncsc1-c1cc2cnc(Nc3ccc(N4CCNCC4)c(F)c3)nc2n(Cc2ccccc2S(C)=O)c1=O ; CS(=O)c1ccccc1Cn1c(=O)c(C#N)cc2cnc(Nc3ccc(N4CCNCC4)c(F)c3)nc21]

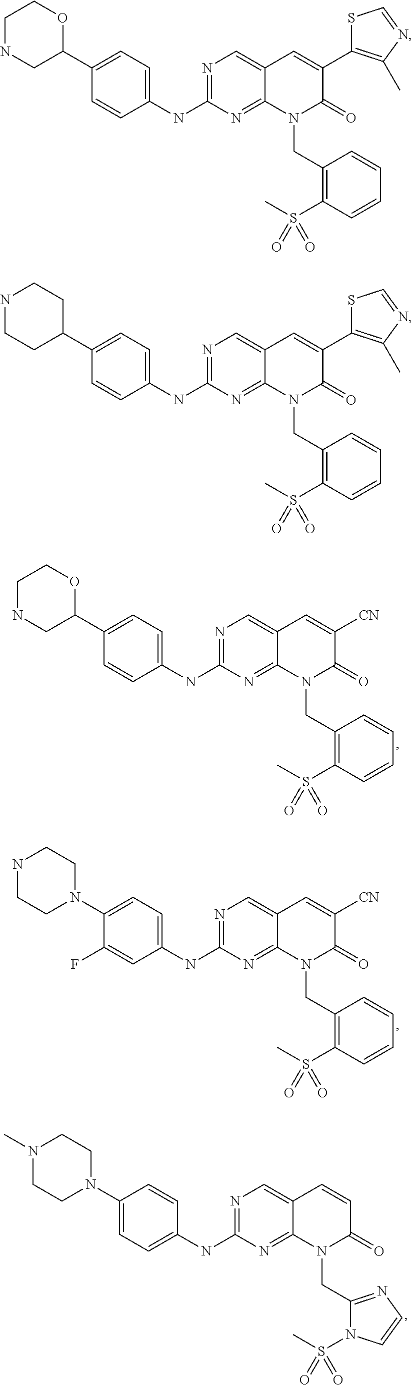 [SMILES: Cc1ncsc1-c1cc2cnc(Nc3ccc(C4CNCCO4)cc3)nc2n(Cc2ccccc2S(C)(=O)=O)c1=O ; Cc1ncsc1-c1cc2cnc(Nc3ccc(C4CCNCC4)cc3)nc2n(Cc2ccccc2S(C)(=O)=O)c1=O ; CS(=O)(=O)c1ccccc1Cn1c(=O)c(C#N)cc2cnc(Nc3ccc(C4CNCCO4)cc3)nc21 ; CS(=O)(=O)c1ccccc1Cn1c(=O)c(C#N)cc2cnc(Nc3ccc(N4CCNCC4)c(F)c3)nc21 ; CN1CCN(c2ccc(Nc3ncc4ccc(=O)n(Cc5nccn5S(C)(=O)=O)c4n3)cc2)CC1]

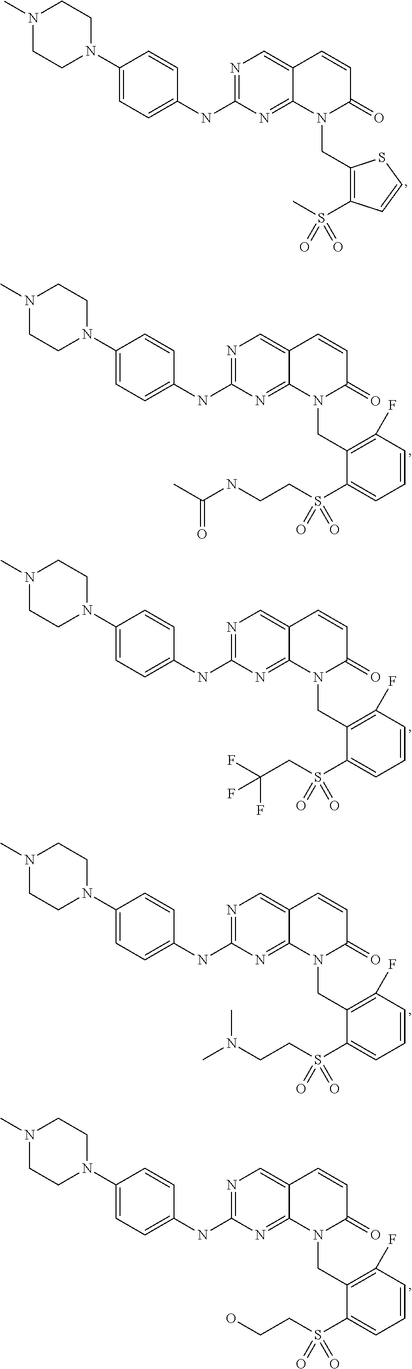 [SMILES: CN1CCN(c2ccc(Nc3ncc4ccc(=O)n(Cc5sccc5S(C)(=O)=O)c4n3)cc2)CC1 ; CC(=O)NCCS(=O)(=O)c1cccc(F)c1Cn1c(=O)ccc2cnc(Nc3ccc(N4CCN(C)CC4)cc3)nc21 ; CN1CCN(c2ccc(Nc3ncc4ccc(=O)n(Cc5c(F)cccc5S(=O)(=O)CC(F)(F)F)c4n3)cc2)CC1 ; CN(C)CCS(=O)(=O)c1cccc(F)c1Cn1c(=O)ccc2cnc(Nc3ccc(N4CCN(C)CC4)cc3)nc21 ; CN1CCN(c2ccc(Nc3ncc4ccc(=O)n(Cc5c(F)cccc5S(=O)(=O)CCO)c4n3)cc2)CC1]

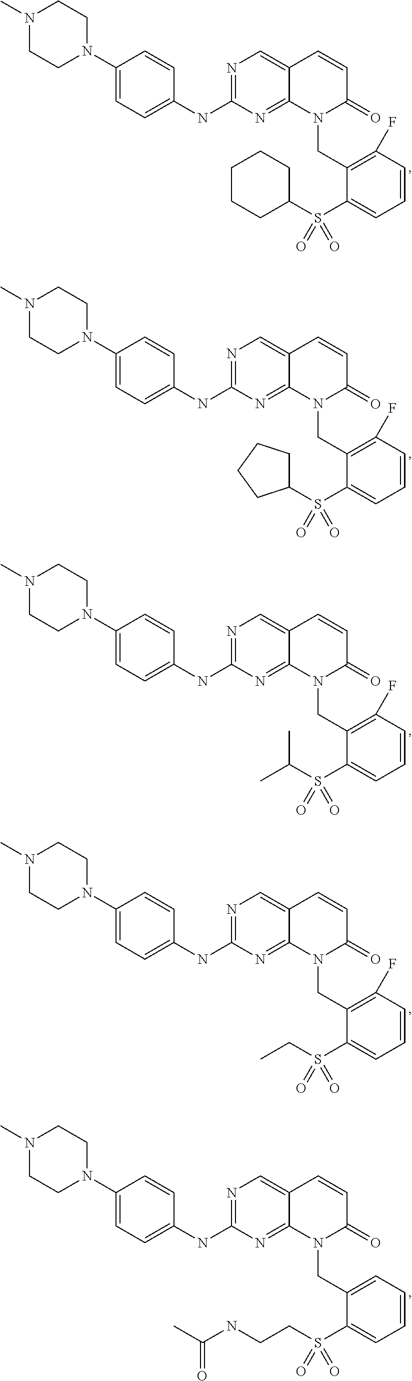 [SMILES: CN1CCN(c2ccc(Nc3ncc4ccc(=O)n(Cc5c(F)cccc5S(=O)(=O)C5CCCCC5)c4n3)cc2)CC1 ; CN1CCN(c2ccc(Nc3ncc4ccc(=O)n(Cc5c(F)cccc5S(=O)(=O)C5CCCC5)c4n3)cc2)CC1 ; CC(C)S(=O)(=O)c1cccc(F)c1Cn1c(=O)ccc2cnc(Nc3ccc(N4CCN(C)CC4)cc3)nc21 ; CCS(=O)(=O)c1cccc(F)c1Cn1c(=O)ccc2cnc(Nc3ccc(N4CCN(C)CC4)cc3)nc21 ; CC(=O)NCCS(=O)(=O)c1ccccc1Cn1c(=O)ccc2cnc(Nc3ccc(N4CCN(C)CC4)cc3)nc21]

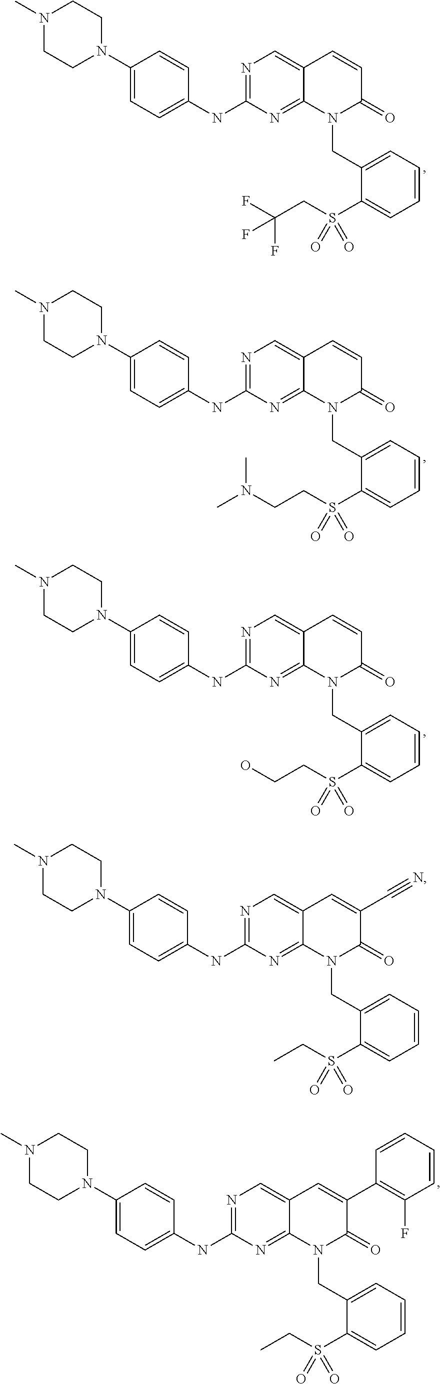 [SMILES: CN1CCN(c2ccc(Nc3ncc4ccc(=O)n(Cc5ccccc5S(=O)(=O)CC(F)(F)F)c4n3)cc2)CC1 ; CN(C)CCS(=O)(=O)c1ccccc1Cn1c(=O)ccc2cnc(Nc3ccc(N4CCN(C)CC4)cc3)nc21 ; CN1CCN(c2ccc(Nc3ncc4ccc(=O)n(Cc5ccccc5S(=O)(=O)CCO)c4n3)cc2)CC1 ; CCS(=O)(=O)c1ccccc1Cn1c(=O)c(C#N)cc2cnc(Nc3ccc(N4CCN(C)CC4)cc3)nc21 ; CCS(=O)(=O)c1ccccc1Cn1c(=O)c(-c2ccccc2F)cc2cnc(Nc3ccc(N4CCN(C)CC4)cc3)nc21]

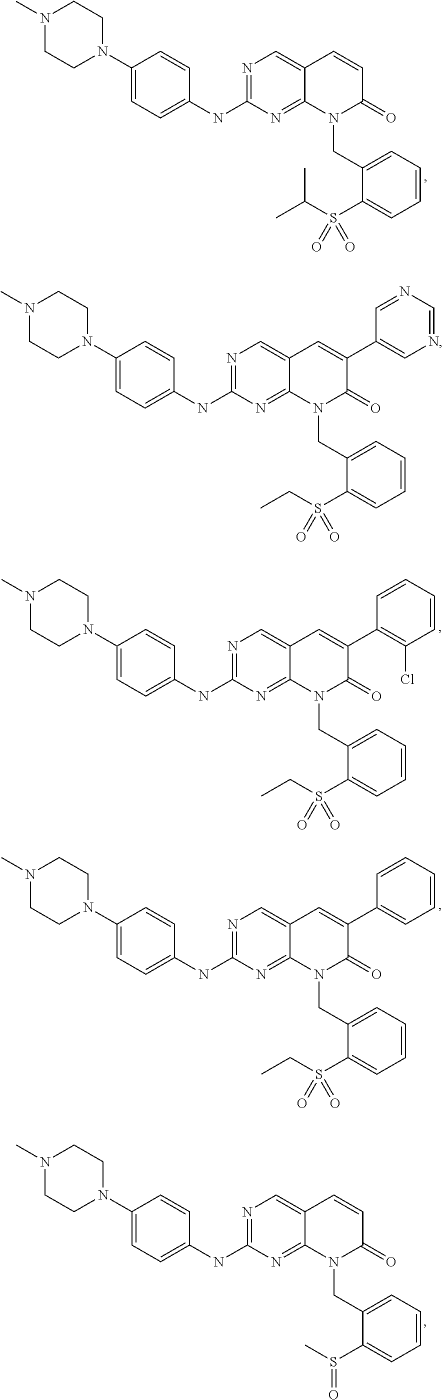 [SMILES: CC(C)S(=O)(=O)c1ccccc1Cn1c(=O)ccc2cnc(Nc3ccc(N4CCN(C)CC4)cc3)nc21 ; CCS(=O)(=O)c1ccccc1Cn1c(=O)c(-c2cncnc2)cc2cnc(Nc3ccc(N4CCN(C)CC4)cc3)nc21 ; CCS(=O)(=O)c1ccccc1Cn1c(=O)c(-c2ccccc2Cl)cc2cnc(Nc3ccc(N4CCN(C)CC4)cc3)nc21 ; CCS(=O)(=O)c1ccccc1Cn1c(=O)c(-c2ccccc2)cc2cnc(Nc3ccc(N4CCN(C)CC4)cc3)nc21 ; CN1CCN(c2ccc(Nc3ncc4ccc(=O)n(Cc5ccccc5S(C)=O)c4n3)cc2)CC1]

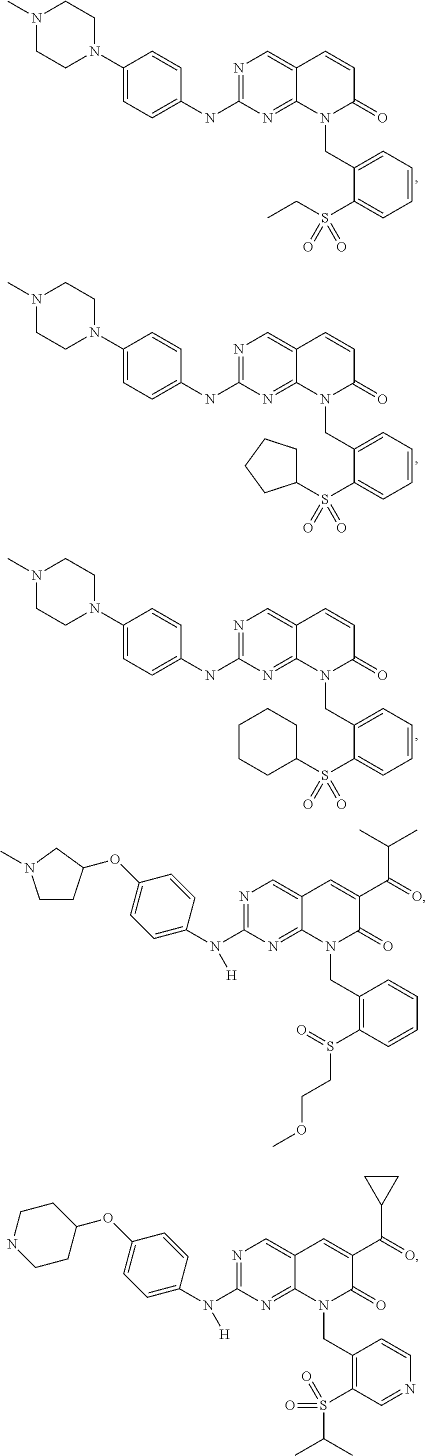

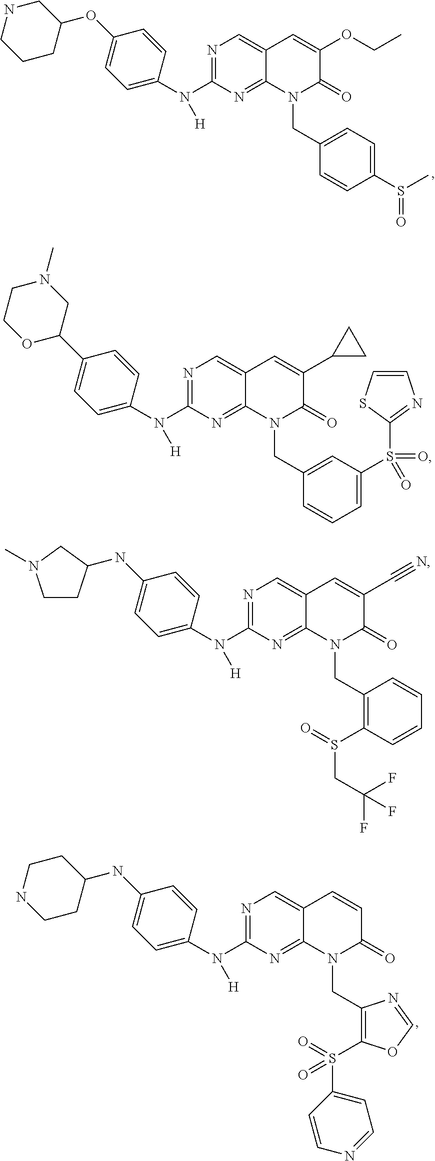

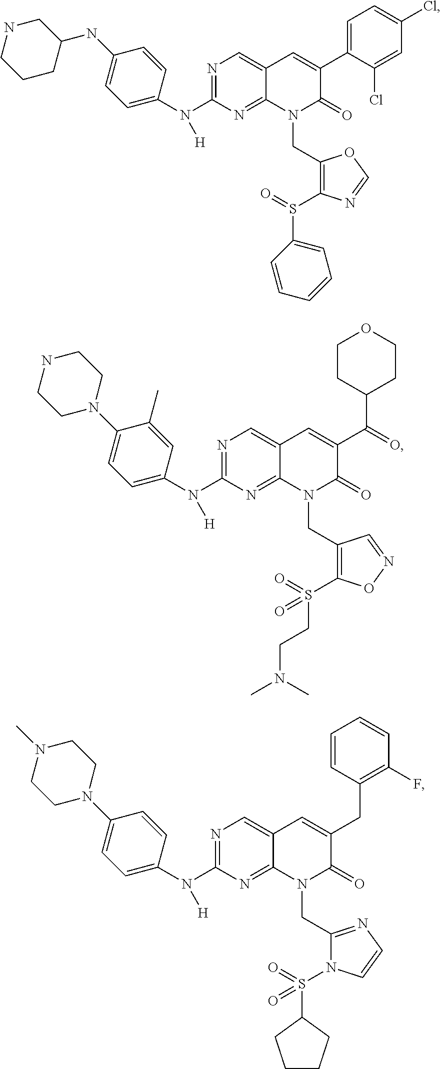

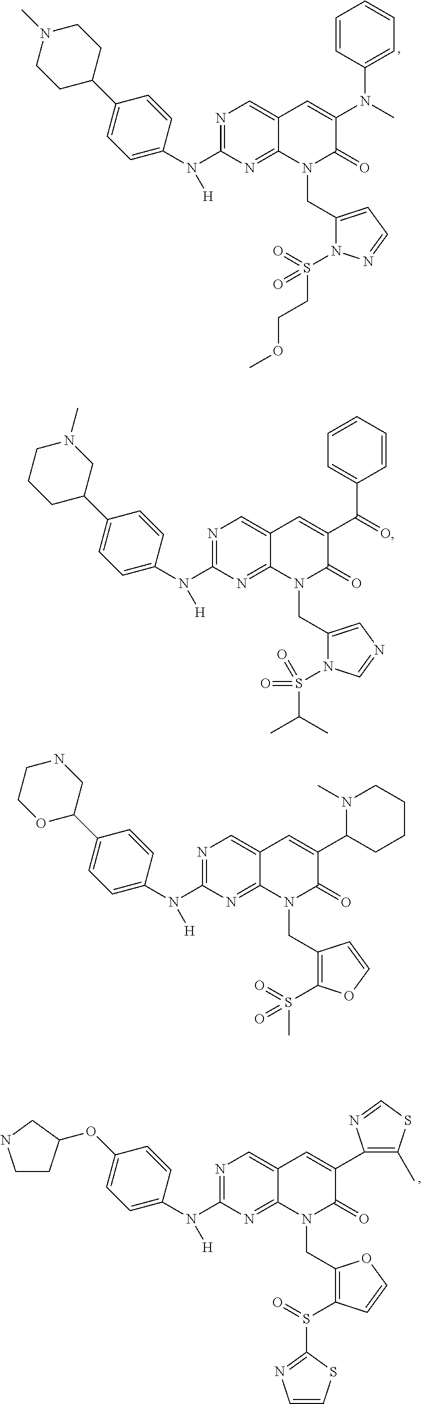

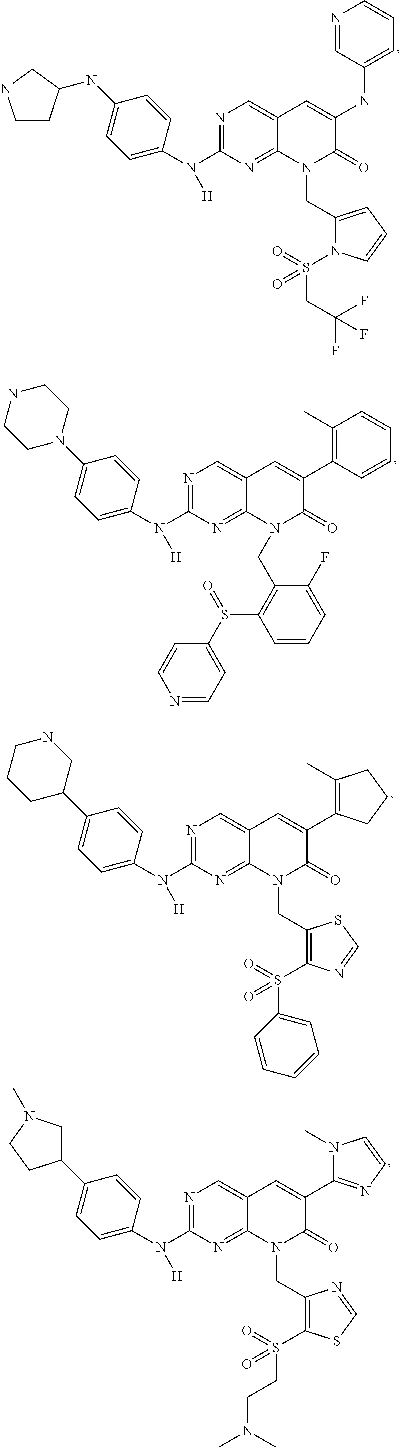 [SMILES: O=c1c(Nc2cccnc2)cc2cnc(Nc3ccc(NC4CCNC4)cc3)nc2n1Cc1cccn1S(=O)(=O)CC(F)(F)F ; Cc1ccccc1-c1cc2cnc(Nc3ccc(N4CCNCC4)cc3)nc2n(Cc2c(F)cccc2S(=O)c2ccncc2)c1=O ; CC1=C(c2cc3cnc(Nc4ccc(C5CCCNC5)cc4)nc3n(Cc3scnc3S(=O)(=O)c3ccccc3)c2=O)CCC1 ; CN(C)CCS(=O)(=O)c1scnc1Cn1c(=O)c(-c2nccn2C)cc2cnc(Nc3ccc(C4CCN(C)C4)cc3)nc21]

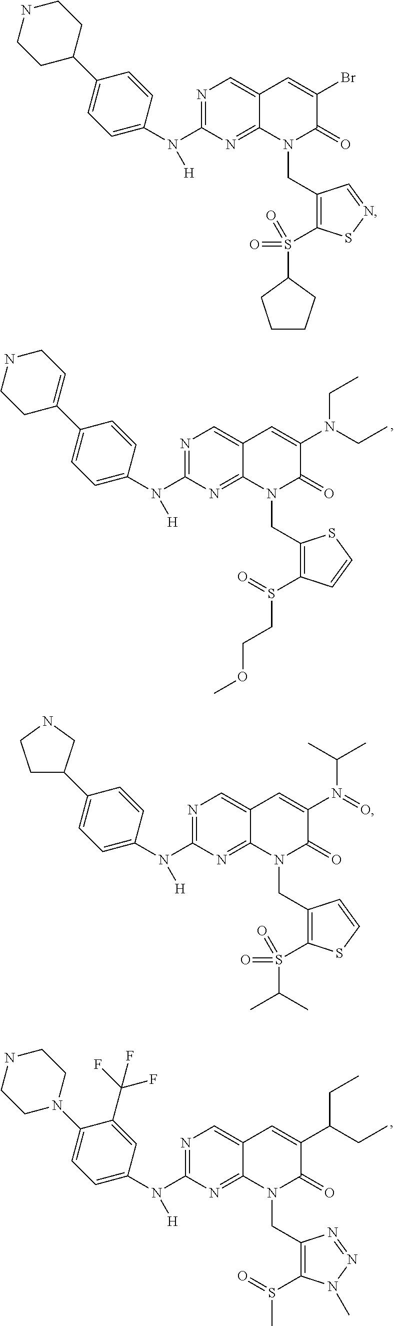

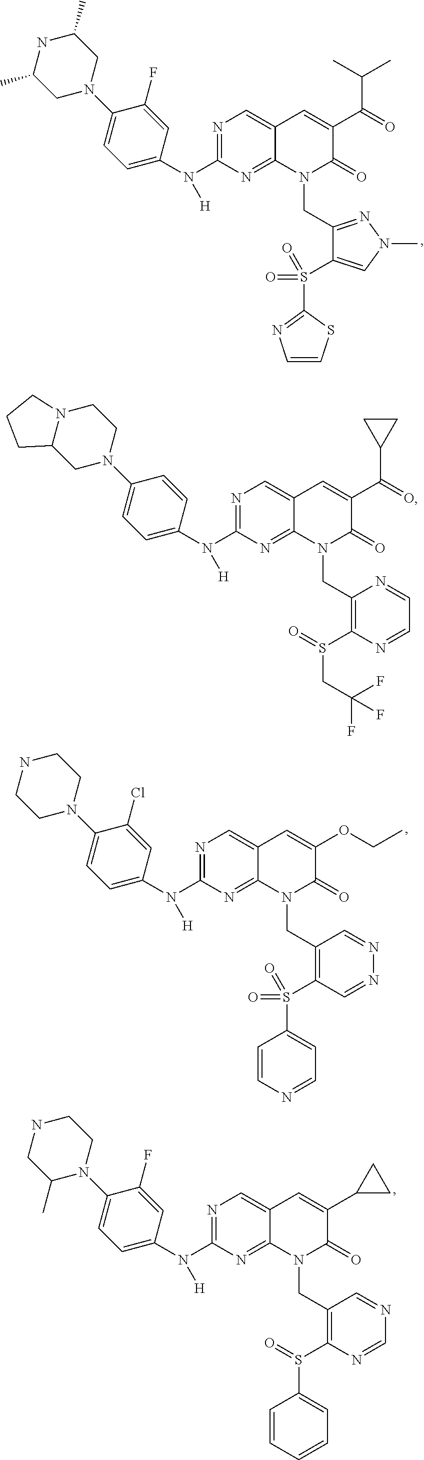

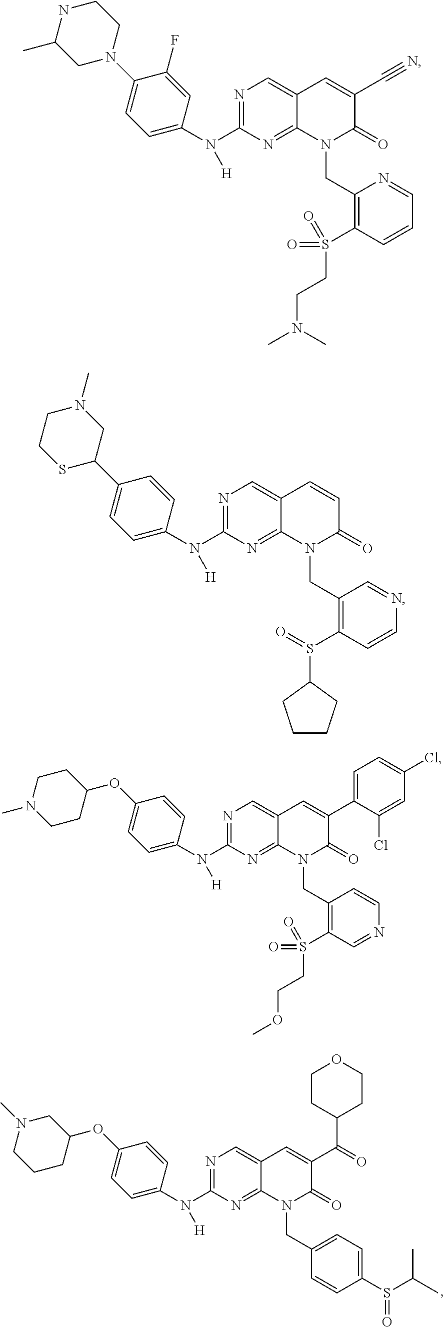 [SMILES: CC1CN(c2ccc(Nc3ncc4cc(C#N)c(=O)n(Cc5ncccc5S(=O)(=O)CCN(C)C)c4n3)cc2F)CCN1 ; CN1CCSC(c2ccc(Nc3ncc4ccc(=O)n(Cc5cnccc5S(=O)C5CCCC5)c4n3)cc2)C1 ; COCCS(=O)(=O)c1cnccc1Cn1c(=O)c(-c2ccc(Cl)cc2Cl)cc2cnc(Nc3ccc(OC4CCN(C)CC4)cc3)nc21 ; CC(C)S(=O)c1ccc(Cn2c(=O)c(C(=O)C3CCOCC3)cc3cnc(Nc4ccc(OC5CCCN(C)C5)cc4)nc32)cc1]

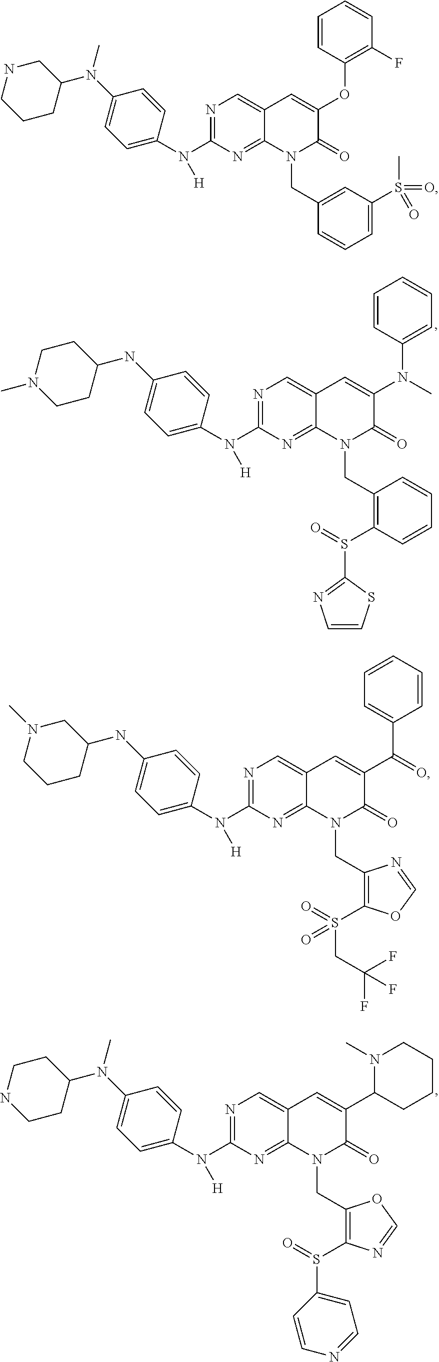

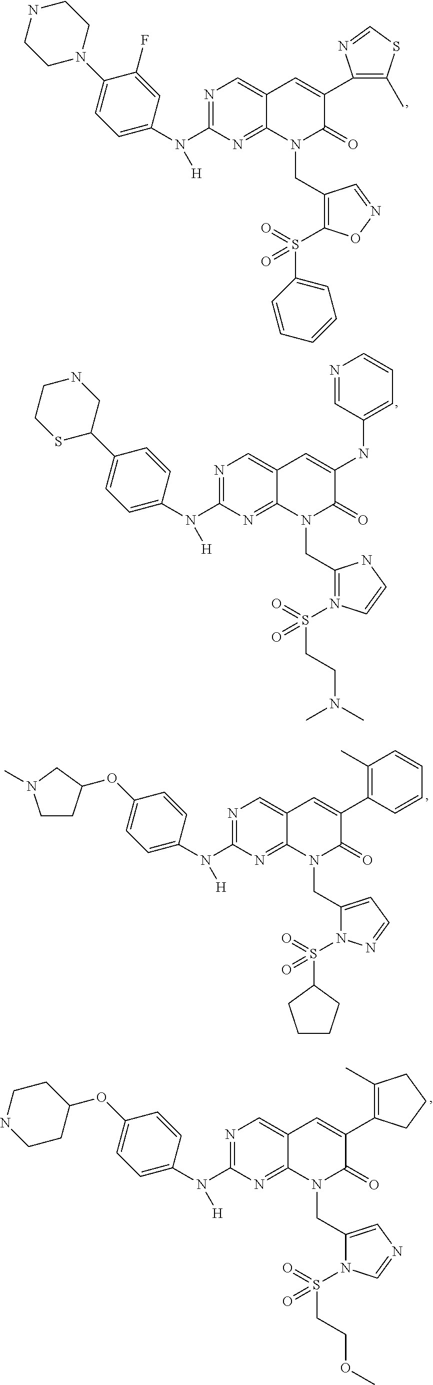

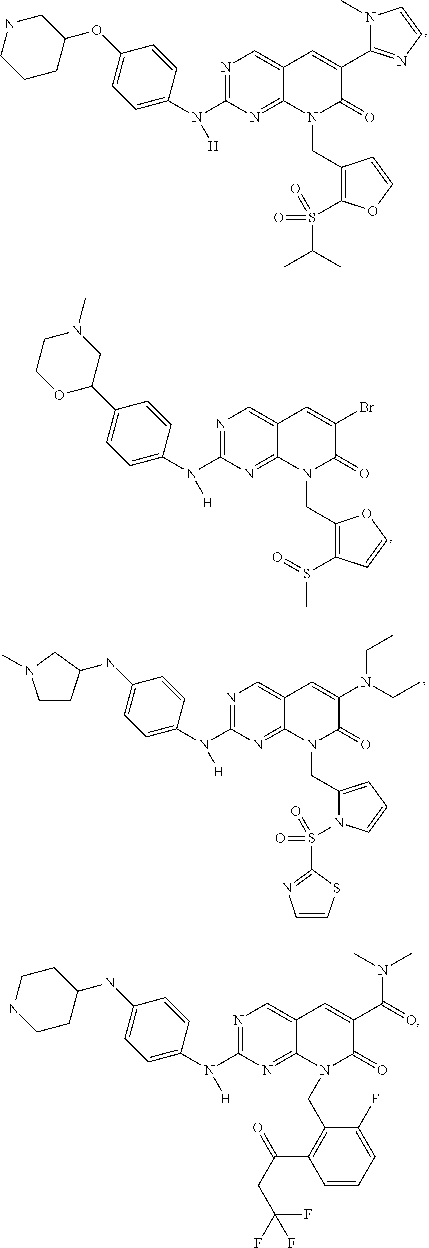

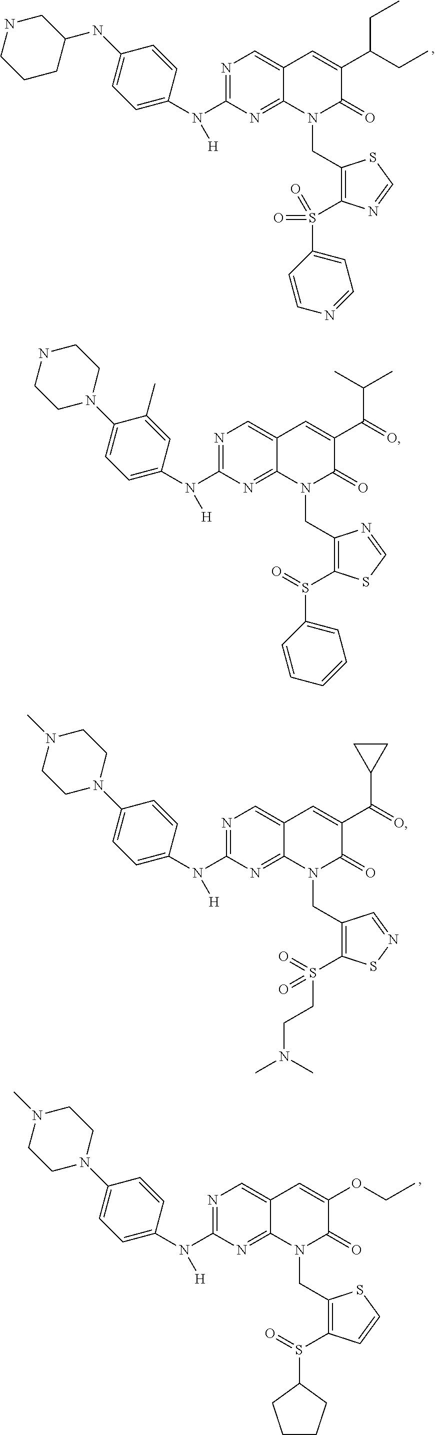

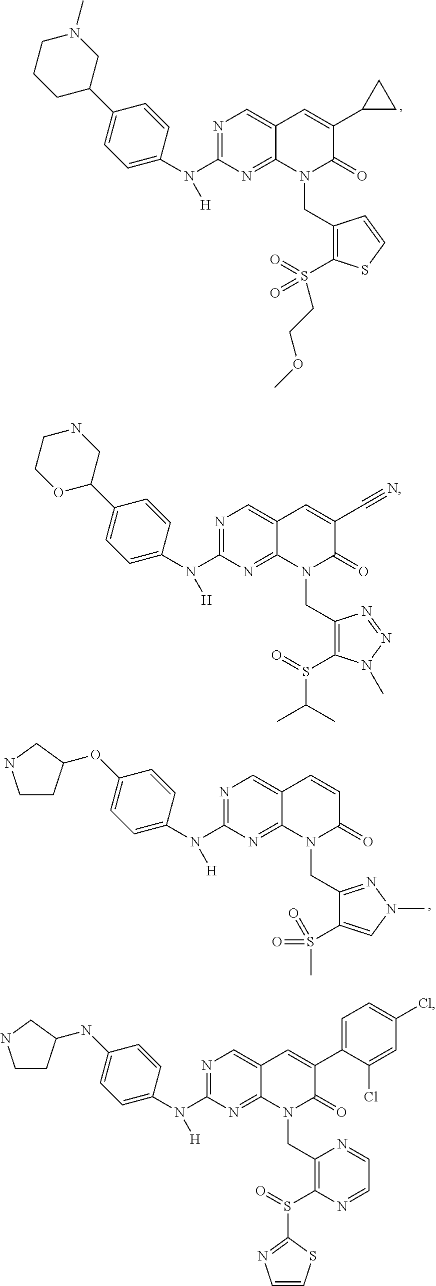 [SMILES: COCCS(=O)(=O)c1sccc1Cn1c(=O)c(C2CC2)cc2cnc(Nc3ccc(C4CCCN(C)C4)cc3)nc21 ; CC(C)S(=O)c1c(Cn2c(=O)c(C#N)cc3cnc(Nc4ccc(C5CNCCO5)cc4)nc32)nnn1C ; Cn1cc(S(C)(=O)=O)c(Cn2c(=O)ccc3cnc(Nc4ccc(OC5CCNC5)cc4)nc32)n1 ; O=c1c(-c2ccc(Cl)cc2Cl)cc2cnc(Nc3ccc(NC4CCNC4)cc3)nc2n1Cc1nccnc1S(=O)c1nccs1]

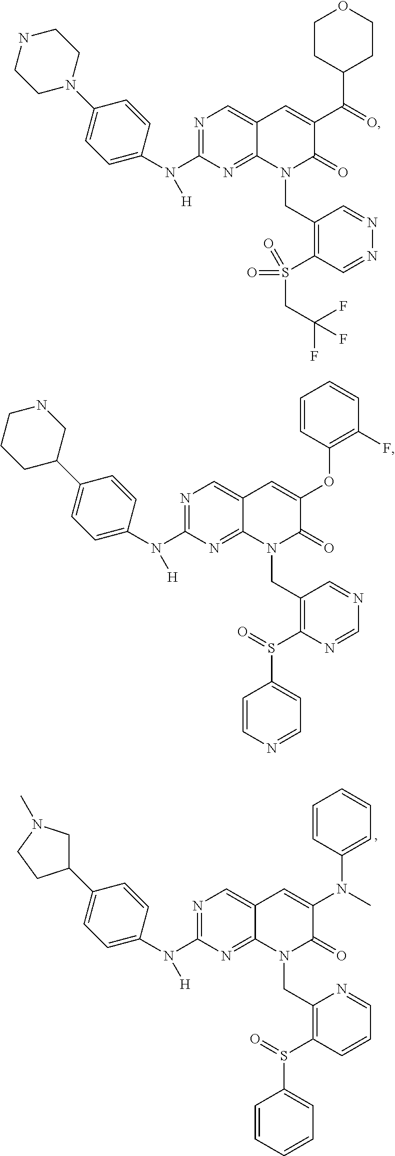

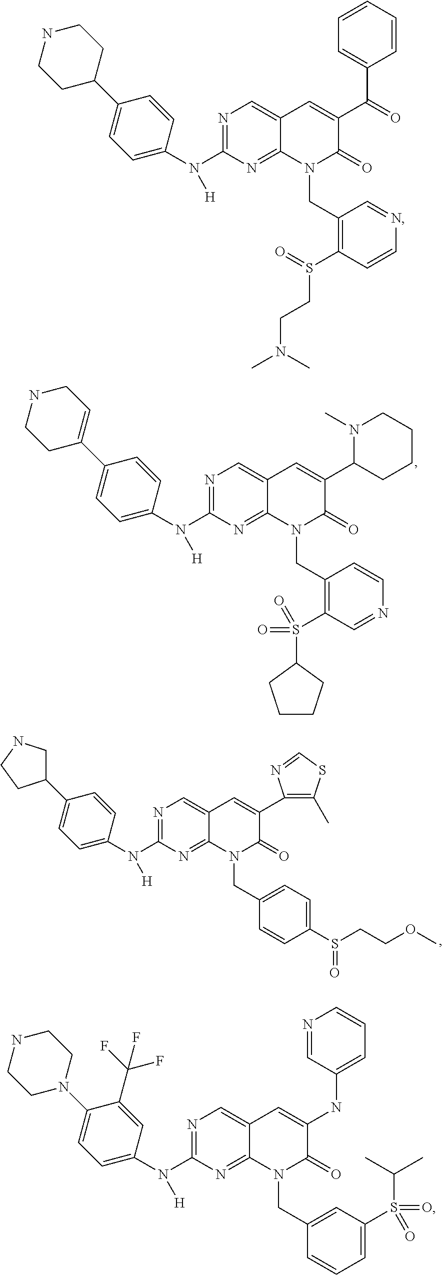

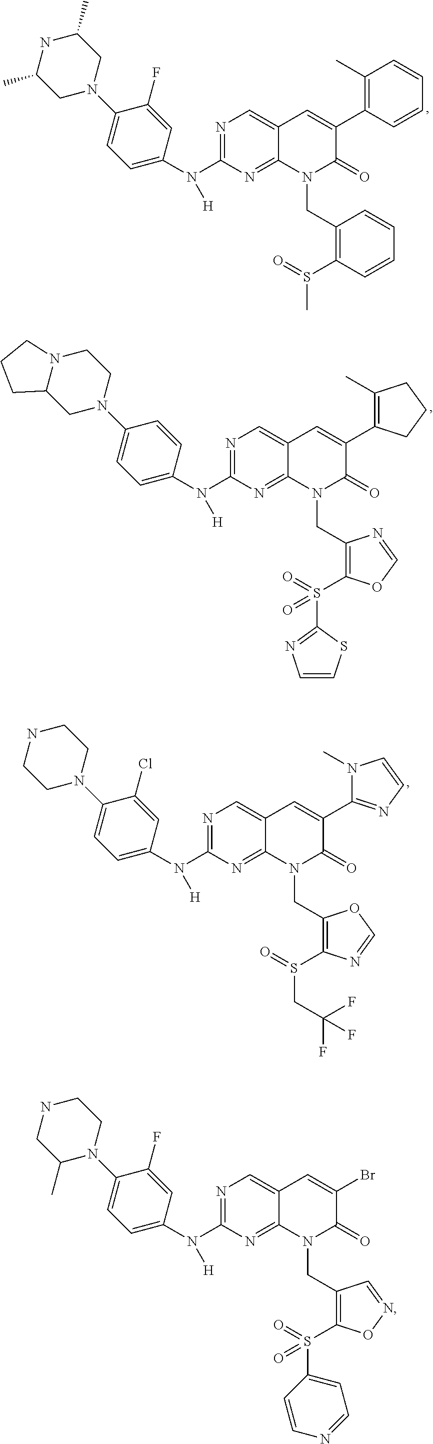 [SMILES: Cc1ccccc1-c1cc2cnc(Nc3ccc(N4C[C@@H](C)N[C@@H](C)C4)c(F)c3)nc2n(Cc2ccccc2S(C)=O)c1=O ; CC1=C(c2cc3cnc(Nc4ccc(N5CCN6CCCC6C5)cc4)nc3n(Cc3ncoc3S(=O)(=O)c3nccs3)c2=O)CCC1 ; Cn1ccnc1-c1cc2cnc(Nc3ccc(N4CCNCC4)c(Cl)c3)nc2n(Cc2ocnc2S(=O)CC(F)(F)F)c1=O ; CC1CNCCN1c1ccc(Nc2ncc3cc(Br)c(=O)n(Cc4cnoc4S(=O)(=O)c4ccncc4)c3n2)cc1F]

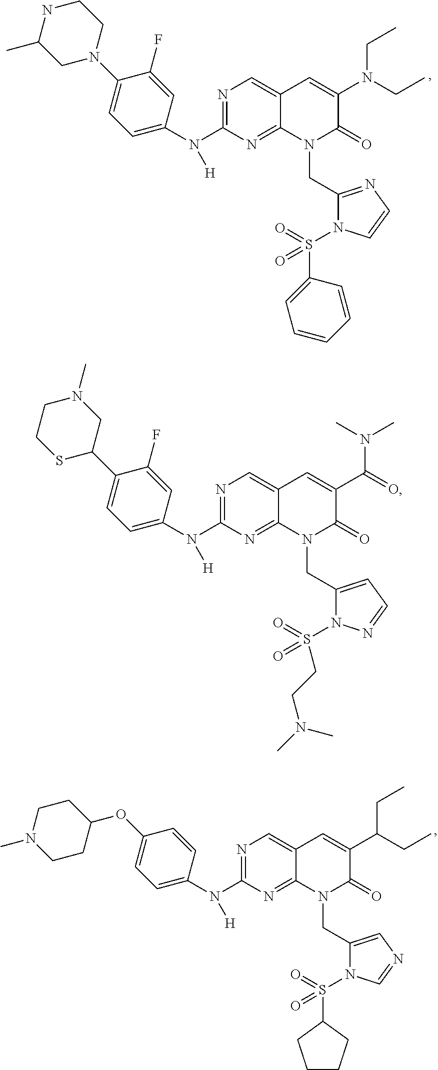

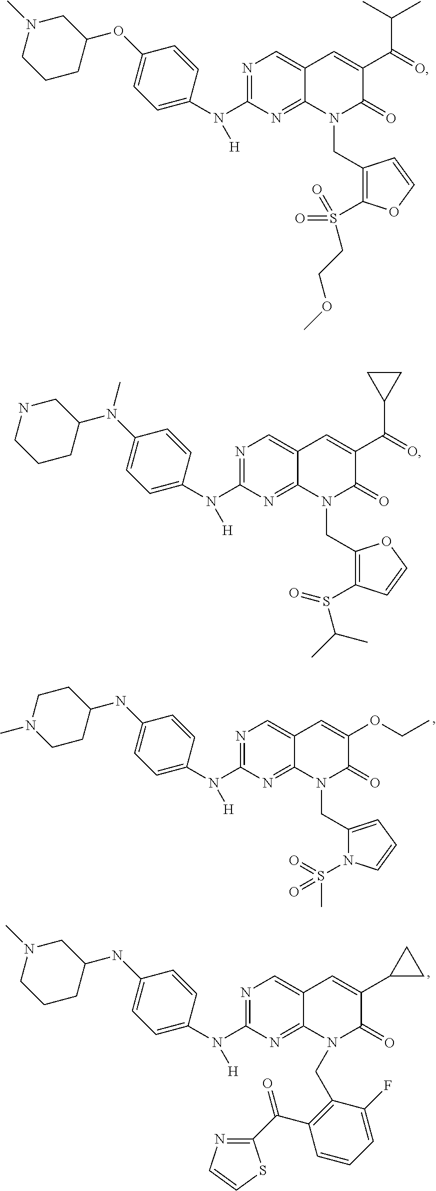

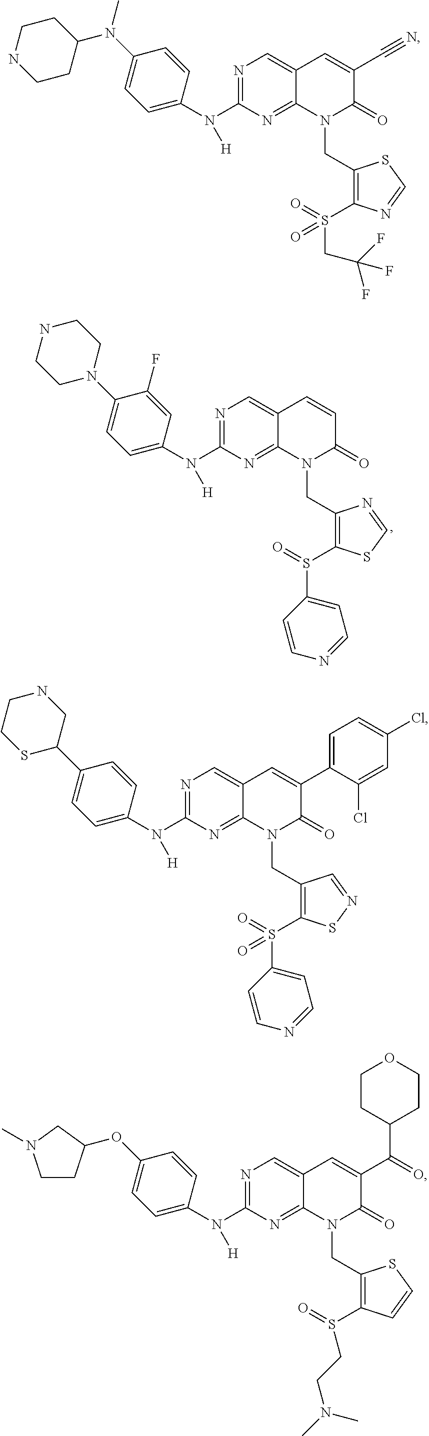 [SMILES: CN(c1ccc(Nc2ncc3cc(C#N)c(=O)n(Cc4scnc4S(=O)(=O)CC(F)(F)F)c3n2)cc1)C1CCNCC1 ; O=c1ccc2cnc(Nc3ccc(N4CCNCC4)c(F)c3)nc2n1Cc1ncsc1S(=O)c1ccncc1 ; O=c1c(-c2ccc(Cl)cc2Cl)cc2cnc(Nc3ccc(C4CNCCS4)cc3)nc2n1Cc1cnsc1S(=O)(=O)c1ccncc1 ; CN(C)CCS(=O)c1ccsc1Cn1c(=O)c(C(=O)C2CCOCC2)cc2cnc(Nc3ccc(OC4CCN(C)C4)cc3)nc21]

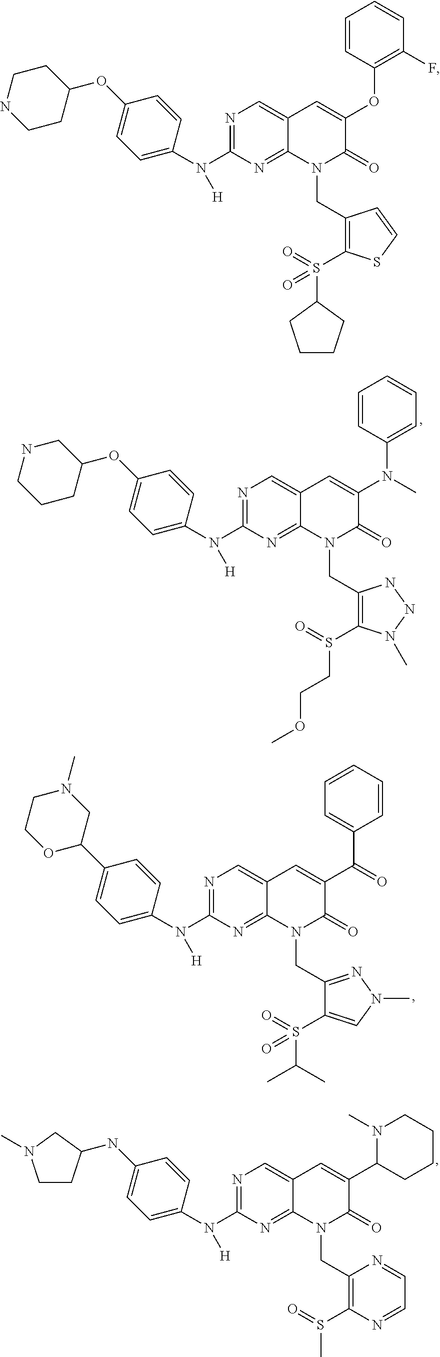

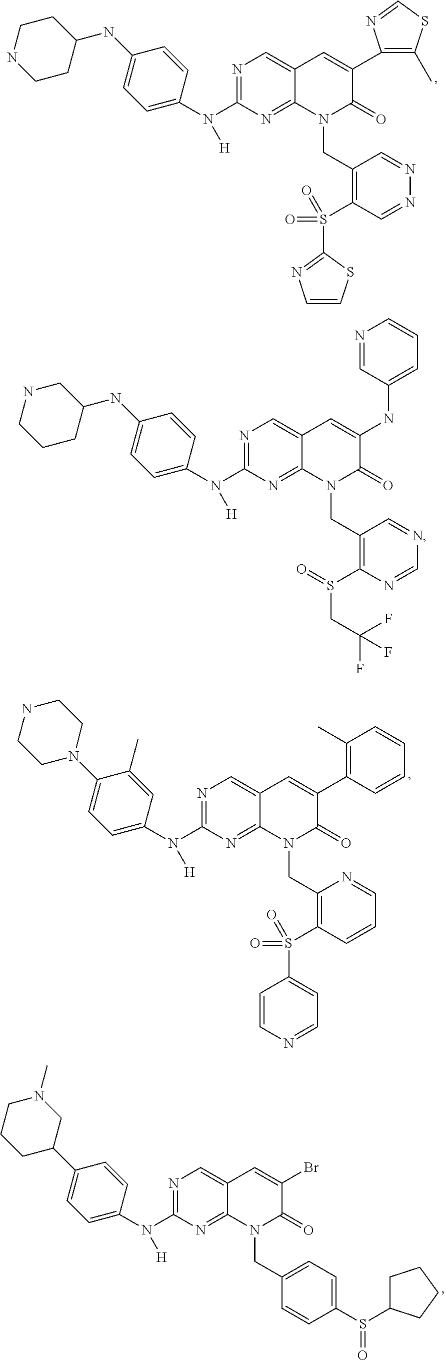

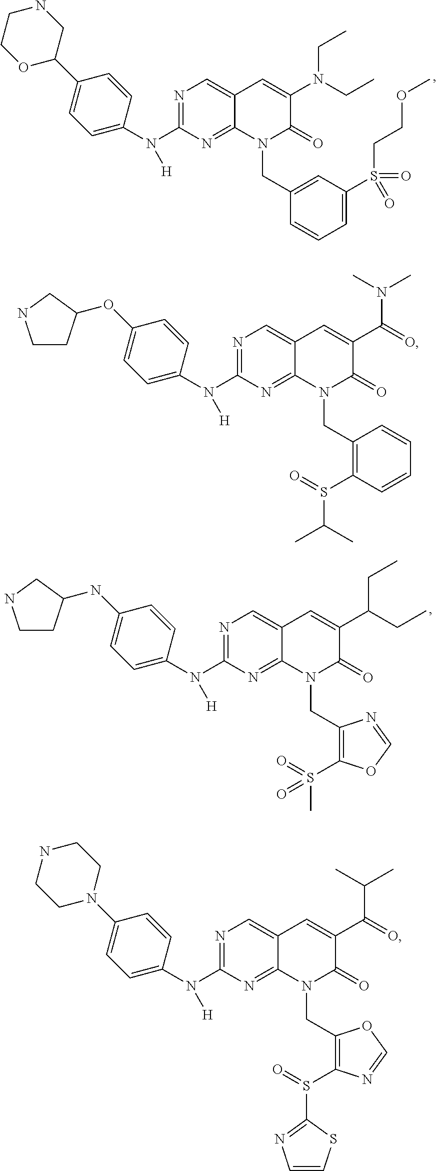

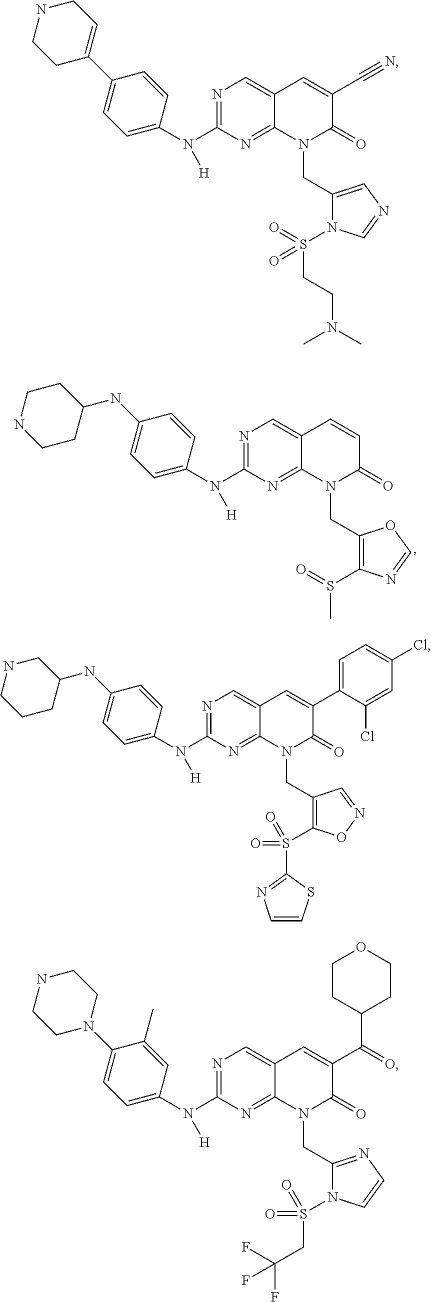

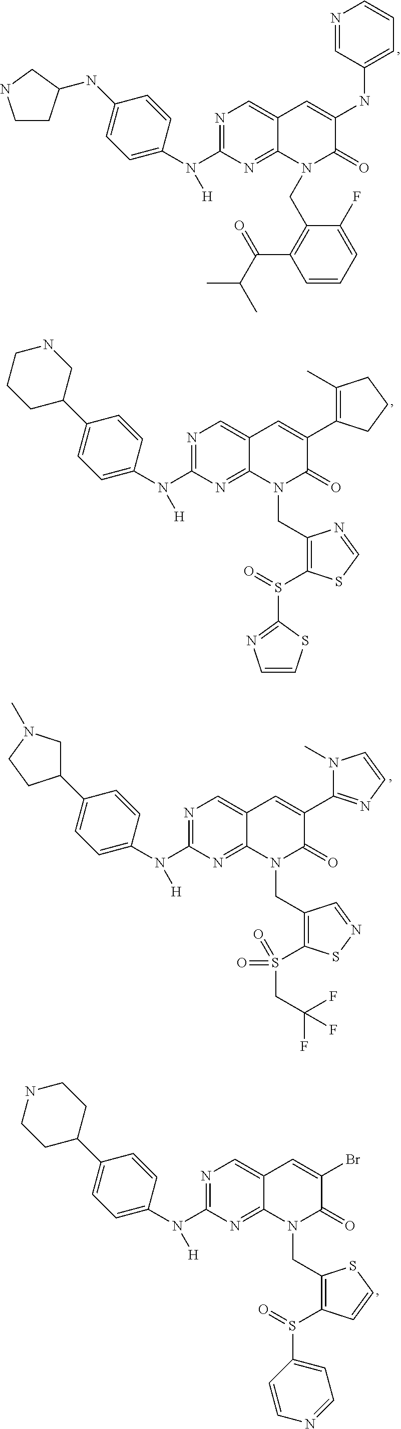 [SMILES: CC(C)C(=O)c1cccc(F)c1Cn1c(=O)c(Nc2cccnc2)cc2cnc(Nc3ccc(NC4CCNC4)cc3)nc21 ; CC1=C(c2cc3cnc(Nc4ccc(C5CCCNC5)cc4)nc3n(Cc3ncsc3S(=O)c3nccs3)c2=O)CCC1 ; CN1CCC(c2ccc(Nc3ncc4cc(-c5nccn5C)c(=O)n(Cc5cnsc5S(=O)(=O)CC(F)(F)F)c4n3)cc2)C1 ; O=c1c(Br)cc2cnc(Nc3ccc(C4CCNCC4)cc3)nc2n1Cc1sccc1S(=O)c1ccncc1]

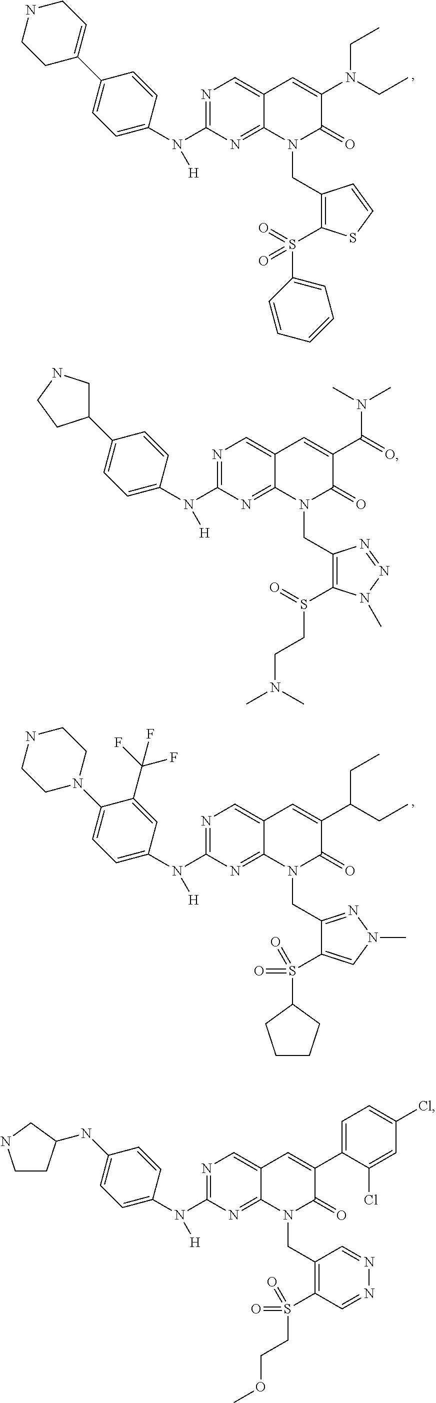 [SMILES: CCN(CC)c1cc2cnc(Nc3ccc(C4=CCNCC4)cc3)nc2n(Cc2ccsc2S(=O)(=O)c2ccccc2)c1=O ; CN(C)CCS(=O)c1c(Cn2c(=O)c(C(=O)N(C)C)cc3cnc(Nc4ccc(C5CCNC5)cc4)nc32)nnn1C ; CCC(CC)c1cc2cnc(Nc3ccc(N4CCNCC4)c(C(F)(F)F)c3)nc2n(Cc2nn(C)cc2S(=O)(=O)C2CCCC2)c1=O ; COCCS(=O)(=O)c1cnncc1Cn1c(=O)c(-c2ccc(Cl)cc2Cl)cc2cnc(Nc3ccc(NC4CCNC4)cc3)nc21]

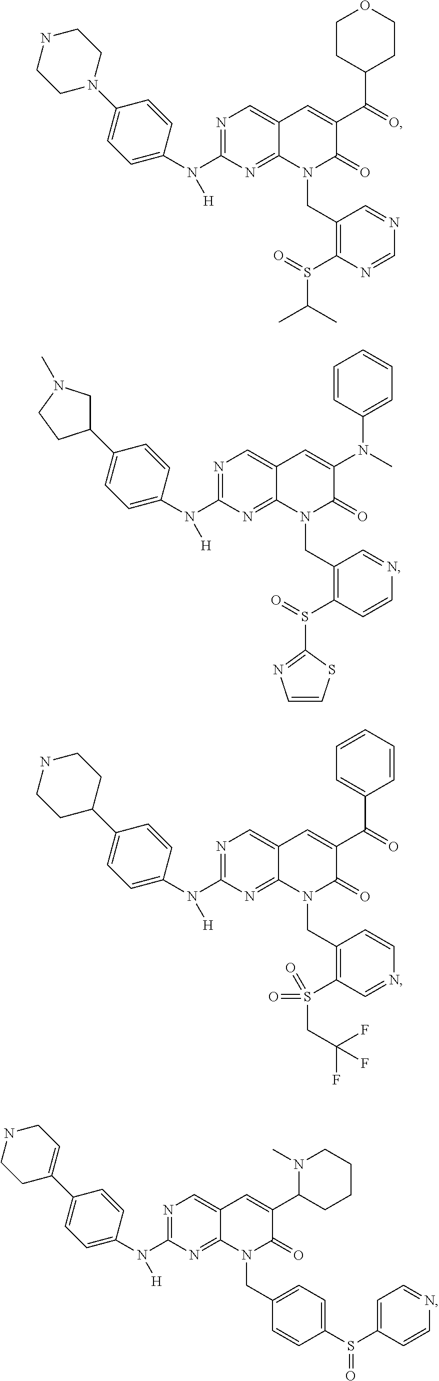

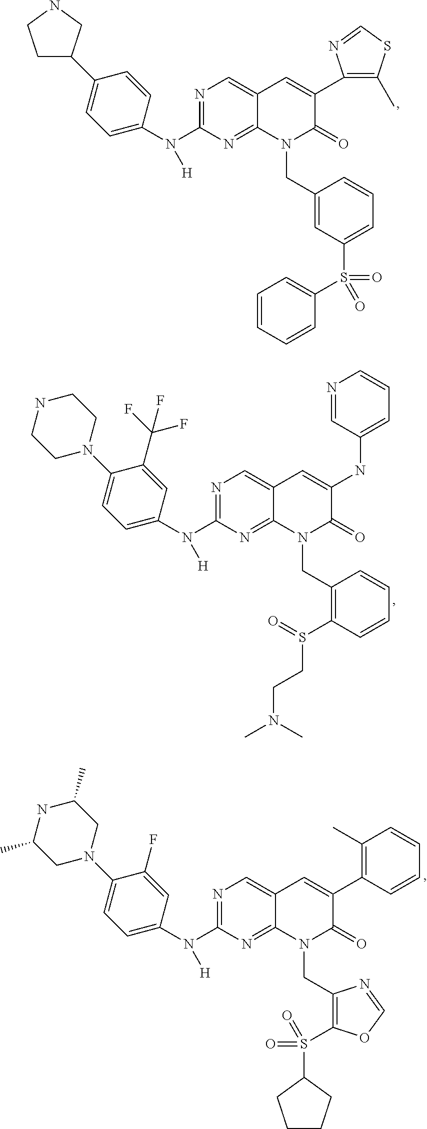

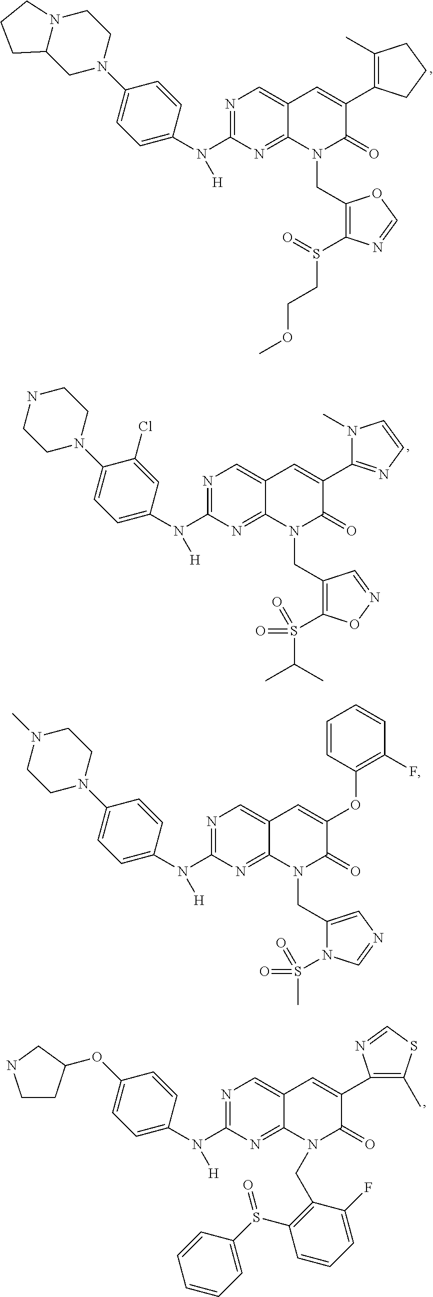

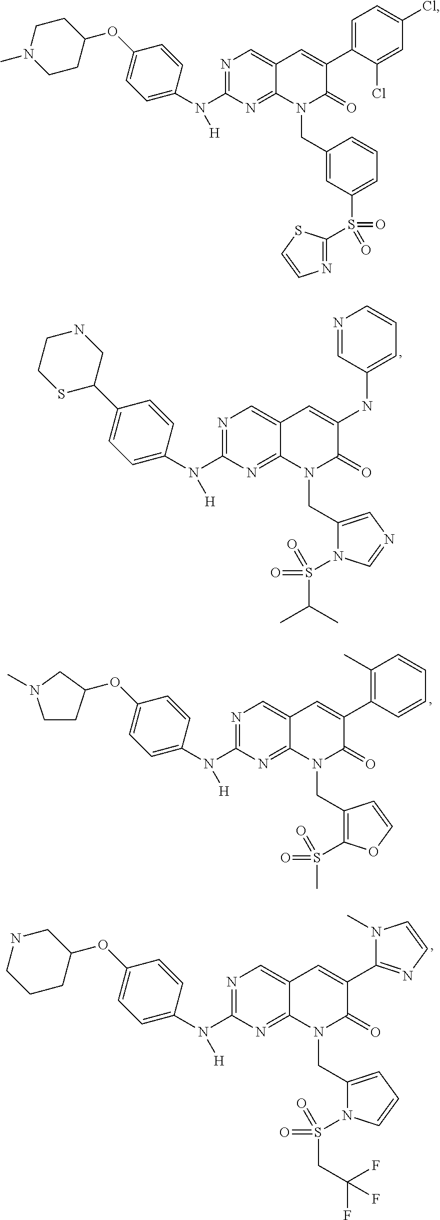

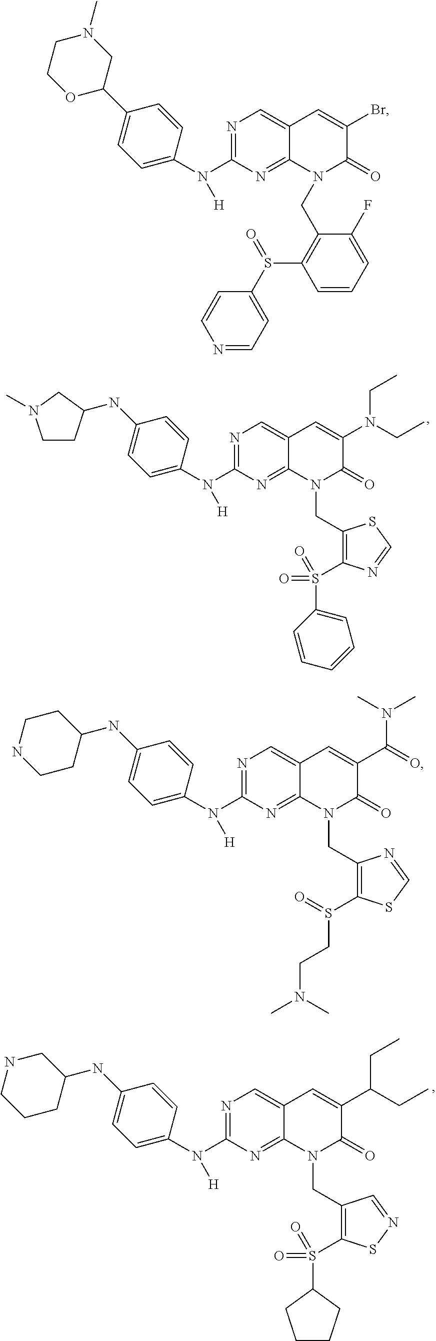

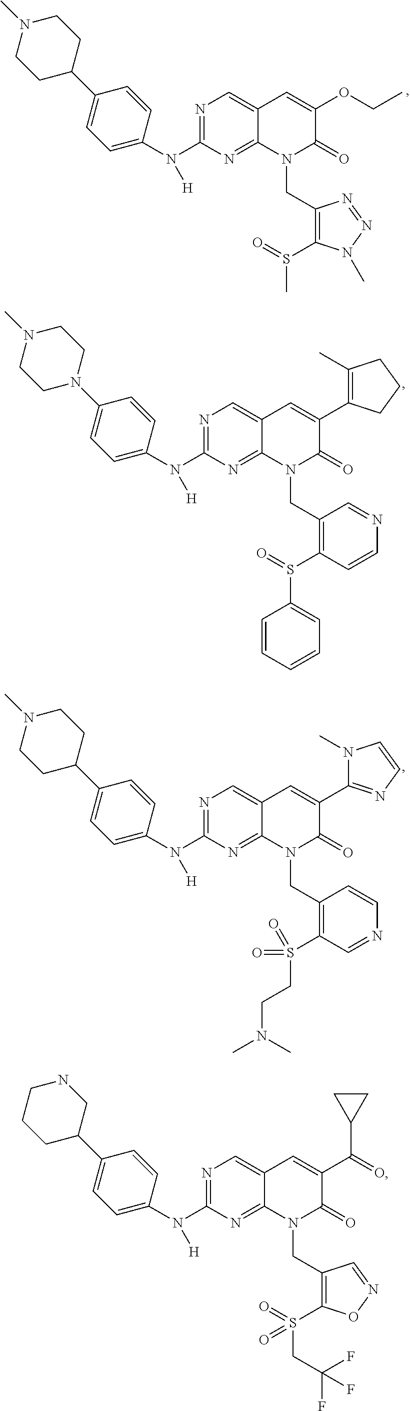

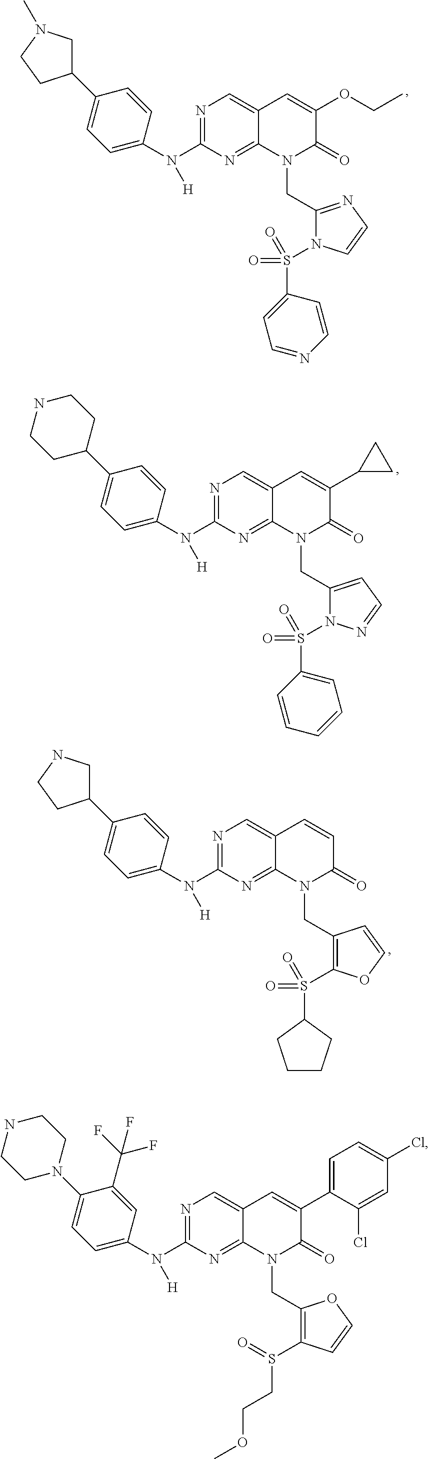 [SMILES: CCOc1cc2cnc(Nc3ccc(C4CCN(C)C4)cc3)nc2n(Cc2nccn2S(=O)(=O)c2ccncc2)c1=O ; O=c1c(C2CC2)cc2cnc(Nc3ccc(C4CCNCC4)cc3)nc2n1Cc1ccnn1S(=O)(=O)c1ccccc1 ; O=c1ccc2cnc(Nc3ccc(C4CCNC4)cc3)nc2n1Cc1ccoc1S(=O)(=O)C1CCCC1 ; COCCS(=O)c1ccoc1Cn1c(=O)c(-c2ccc(Cl)cc2Cl)cc2cnc(Nc3ccc(N4CCNCC4)c(C(F)(F)F)c3)nc21]

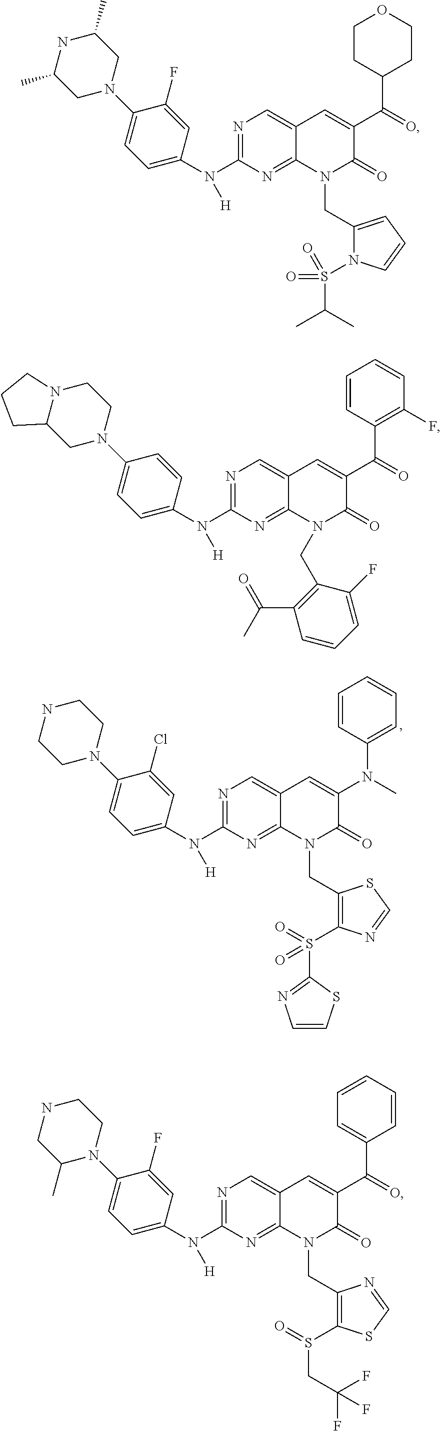

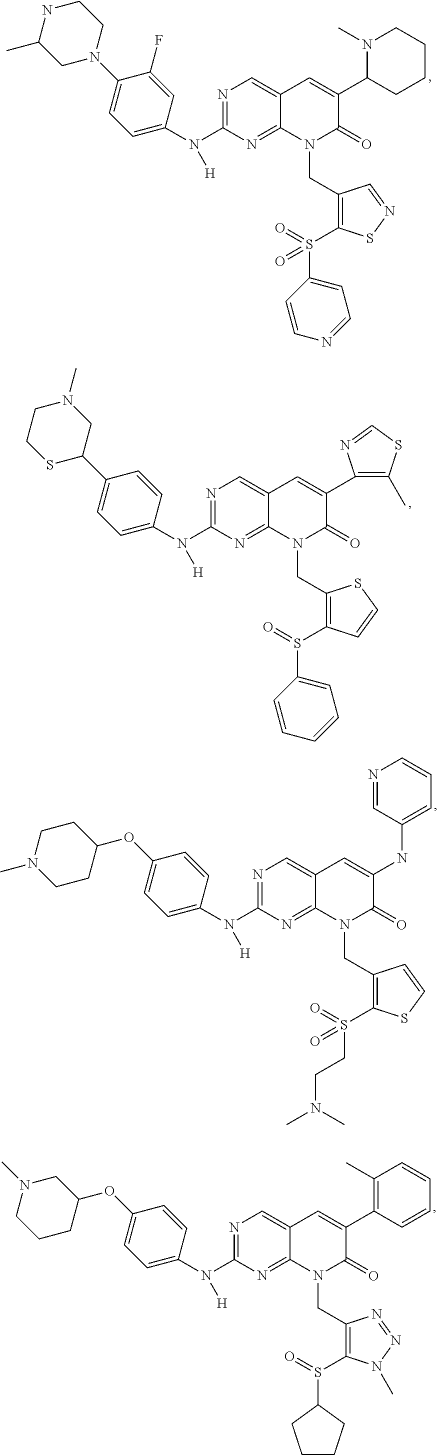

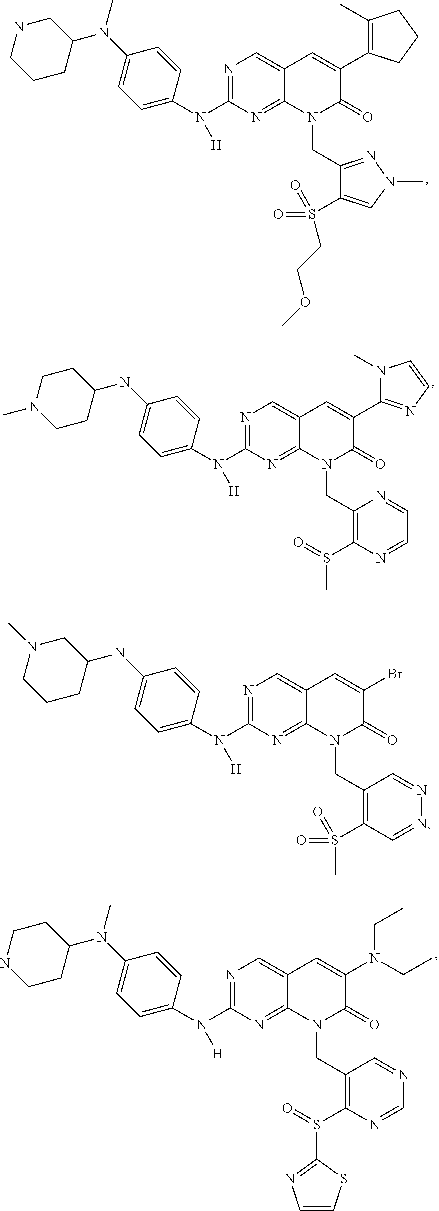

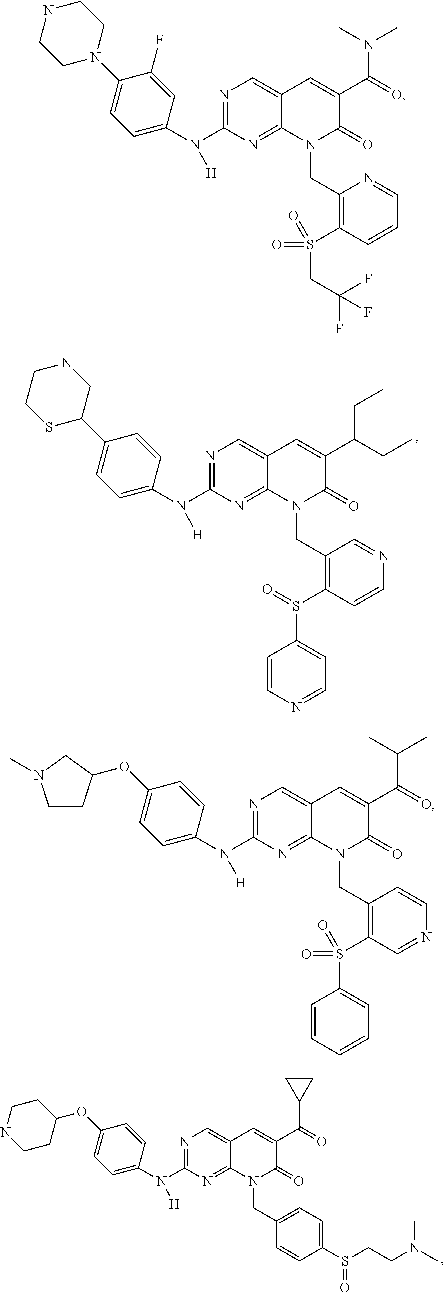

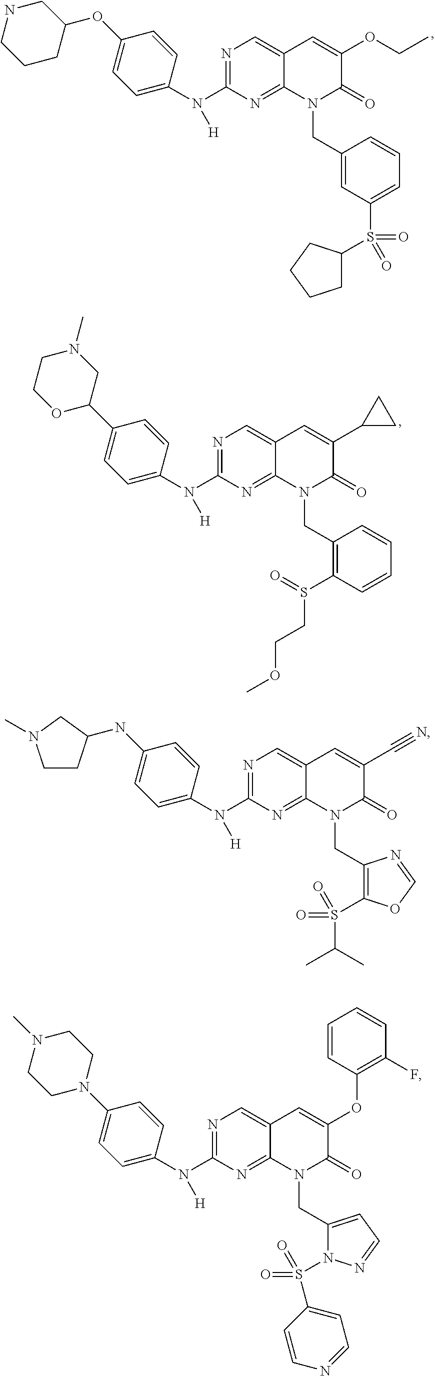

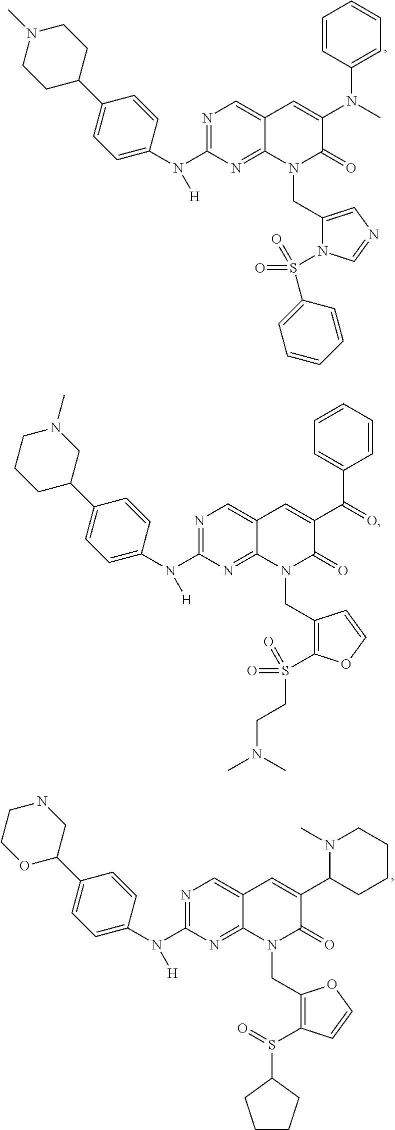

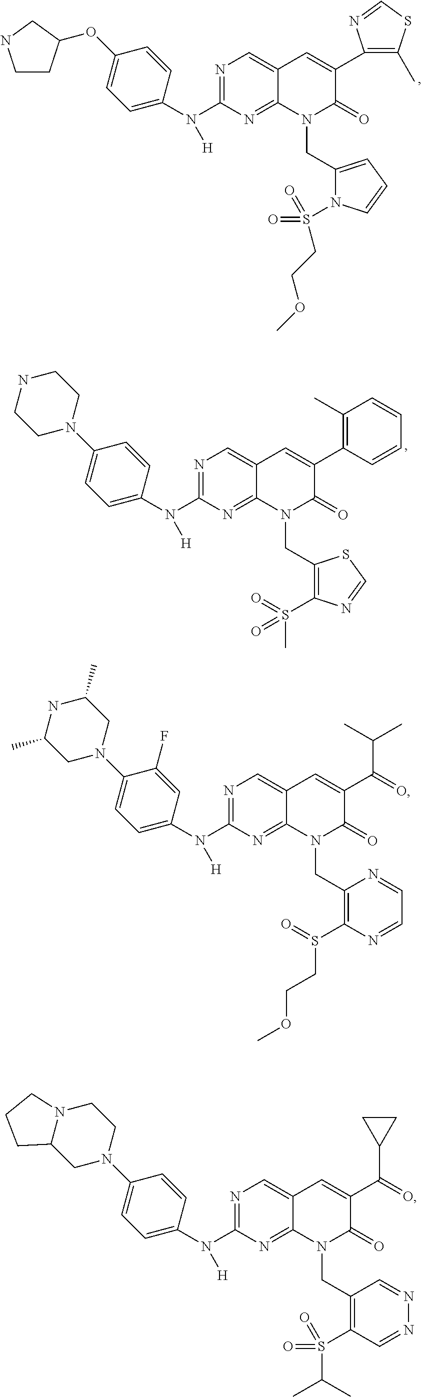

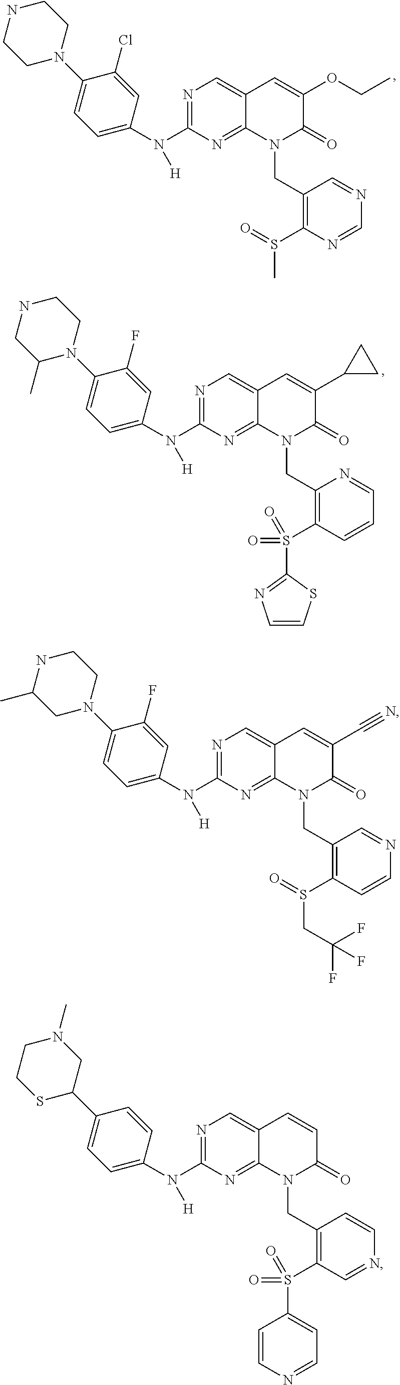

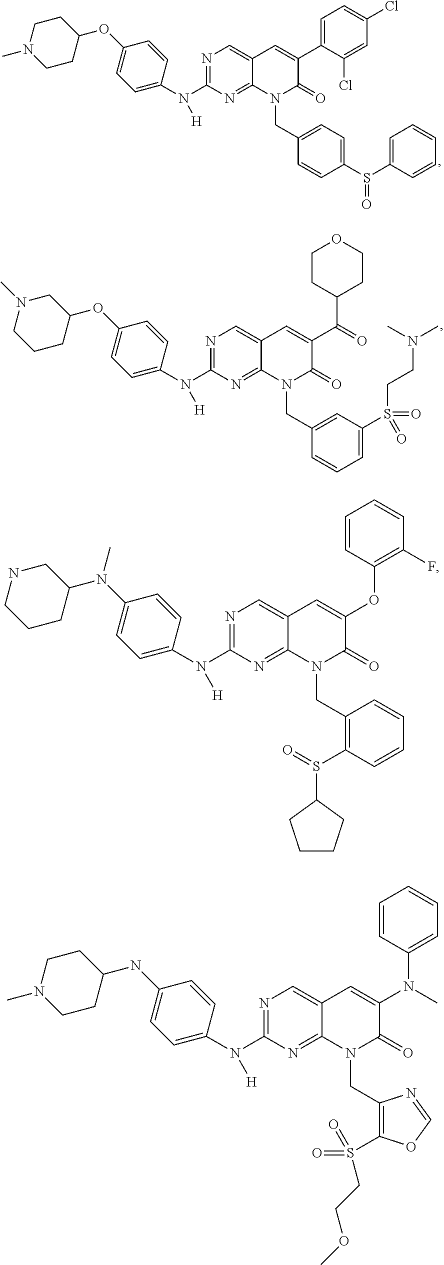

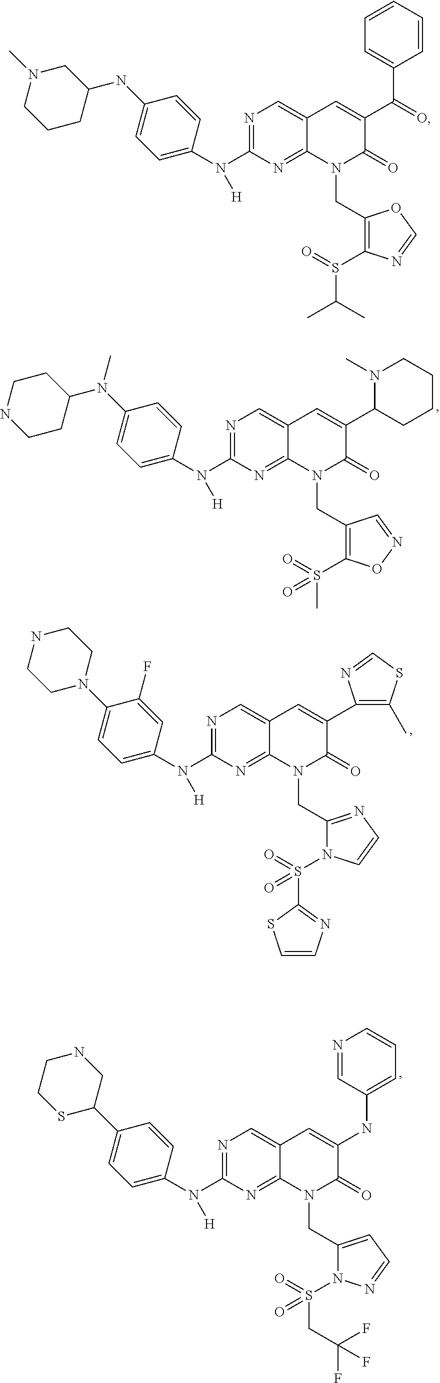

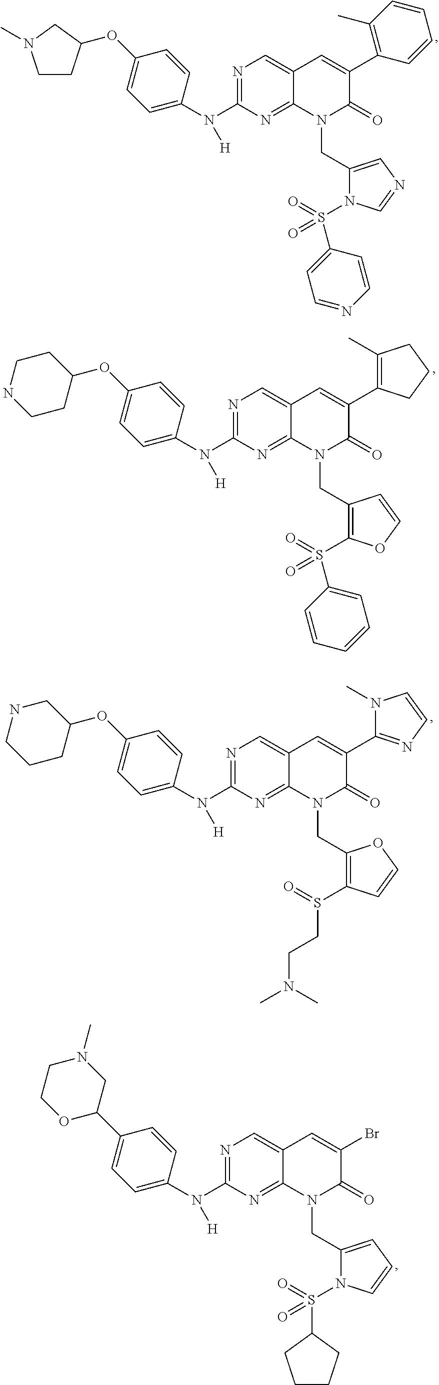

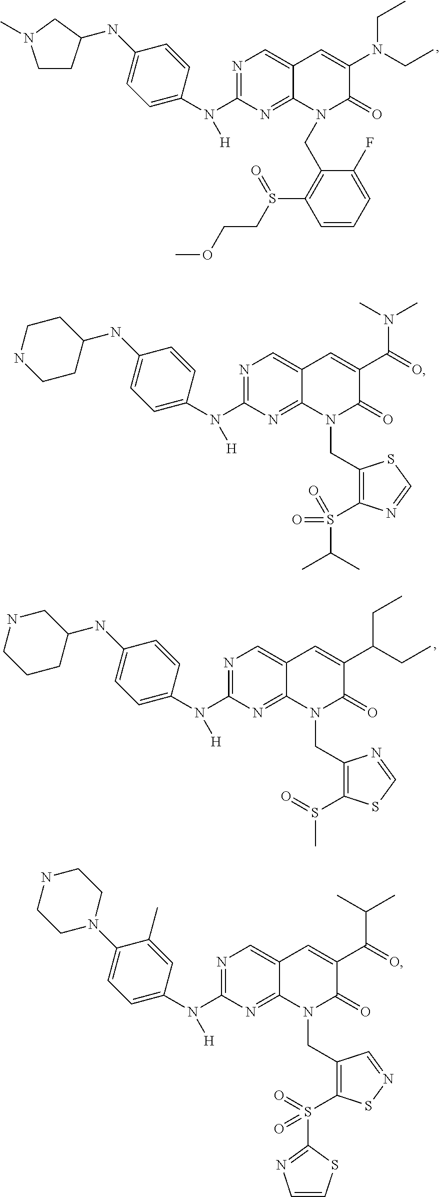

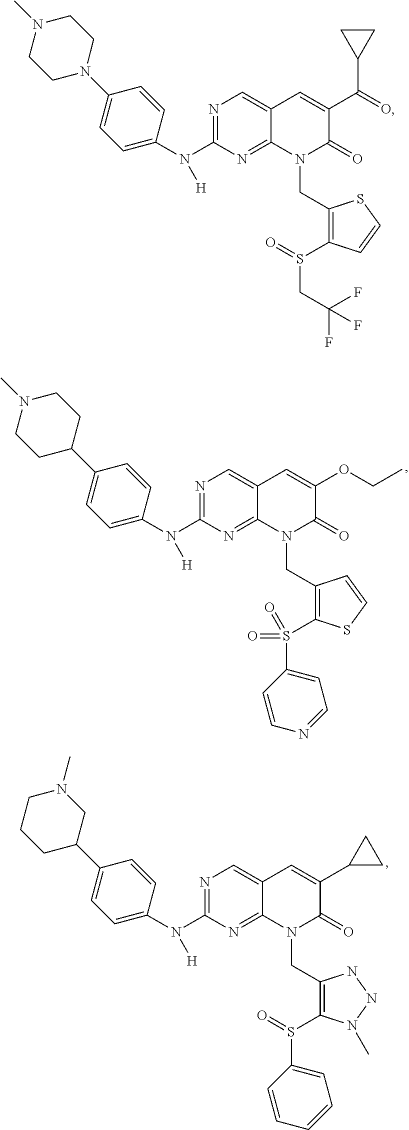 [SMILES: CN1CCN(c2ccc(Nc3ncc4cc(C(=O)C5CC5)c(=O)n(Cc5sccc5S(=O)CC(F)(F)F)c4n3)cc2)CC1 ; CCOc1cc2cnc(Nc3ccc(C4CCN(C)CC4)cc3)nc2n(Cc2ccsc2S(=O)(=O)c2ccncc2)c1=O ; CN1CCCC(c2ccc(Nc3ncc4cc(C5CC5)c(=O)n(CC5=C(S(=O)c6ccccc6)N(C)NN5)c4n3)cc2)C1]

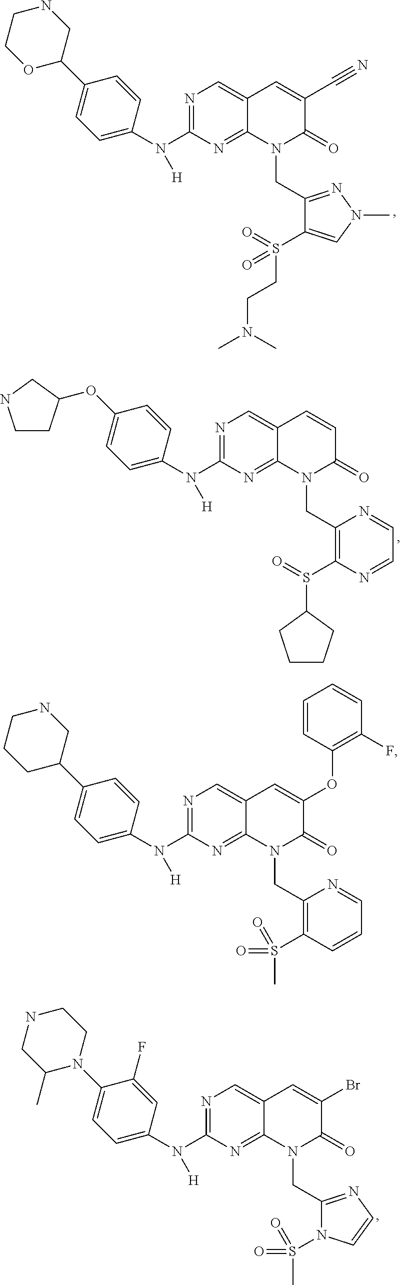

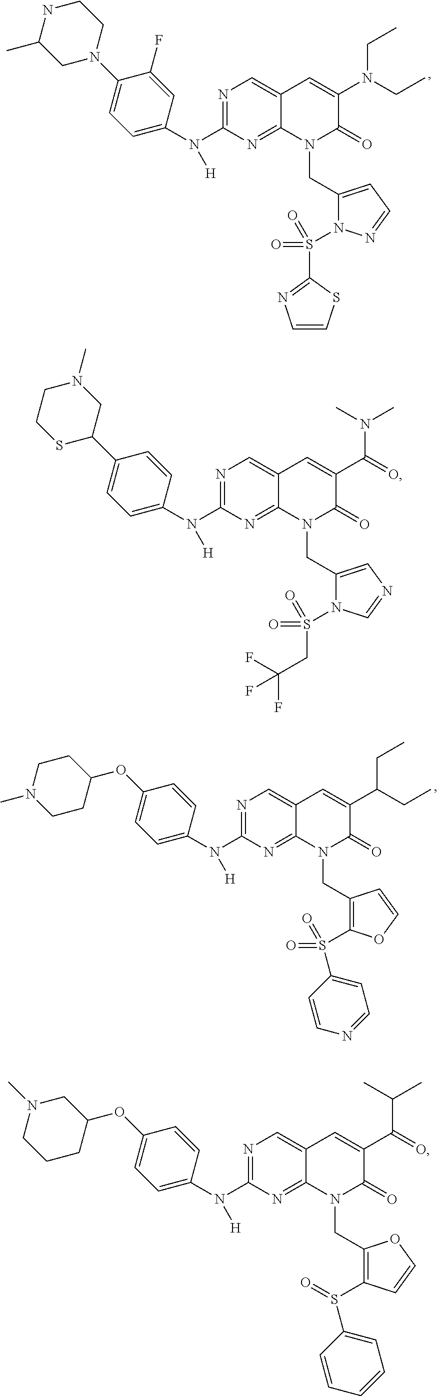

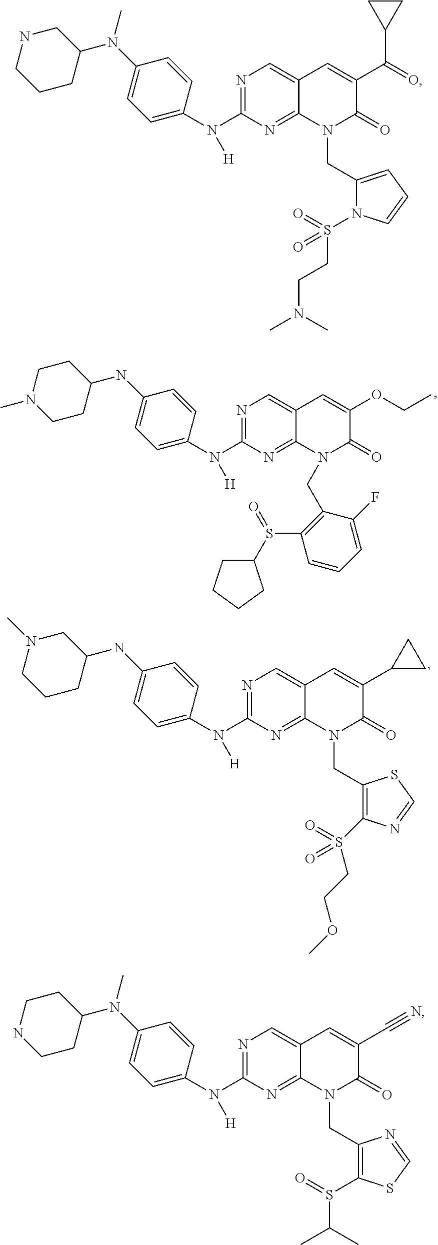

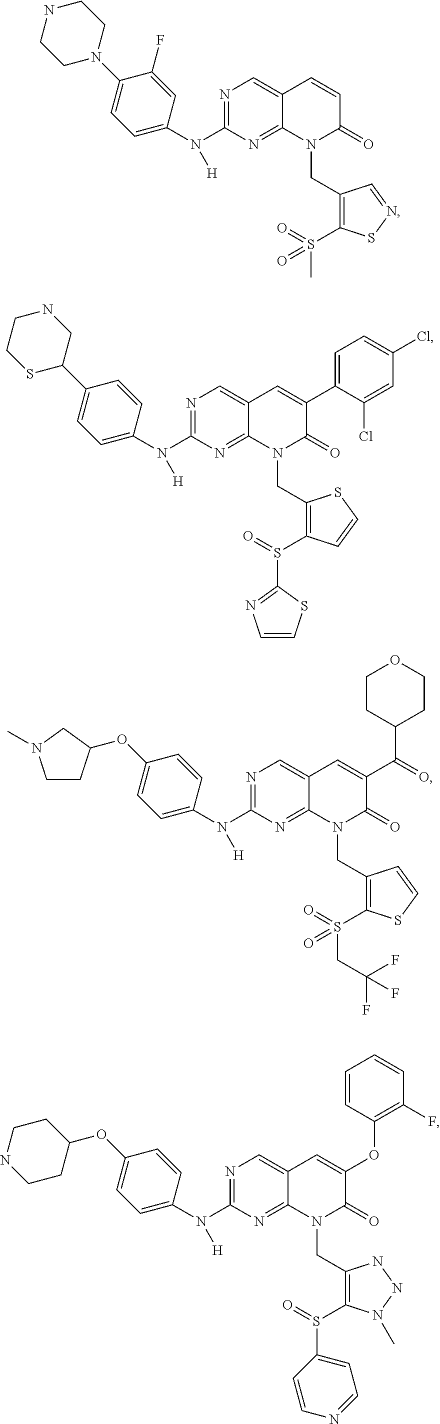

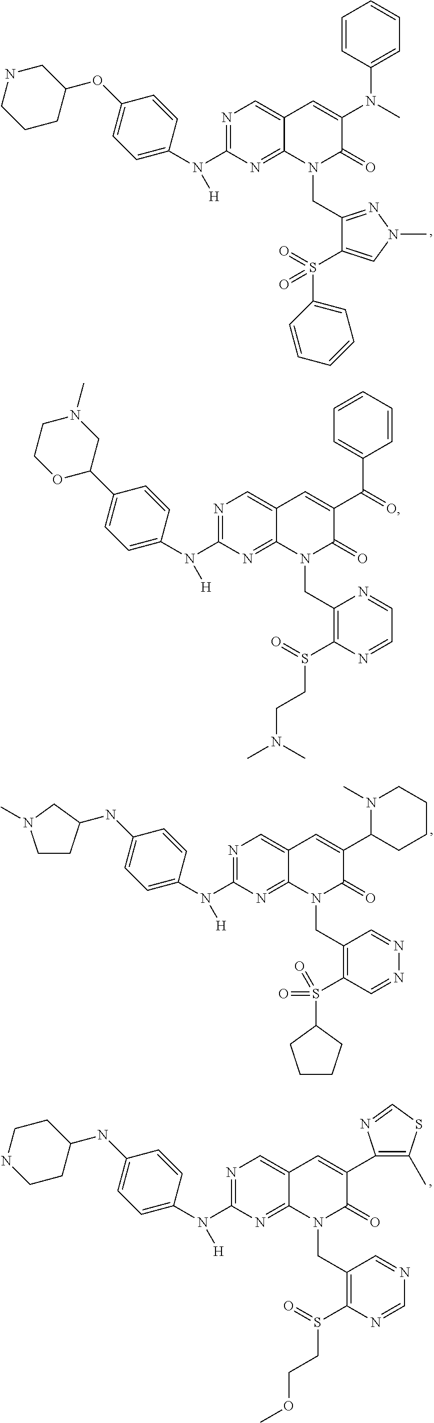

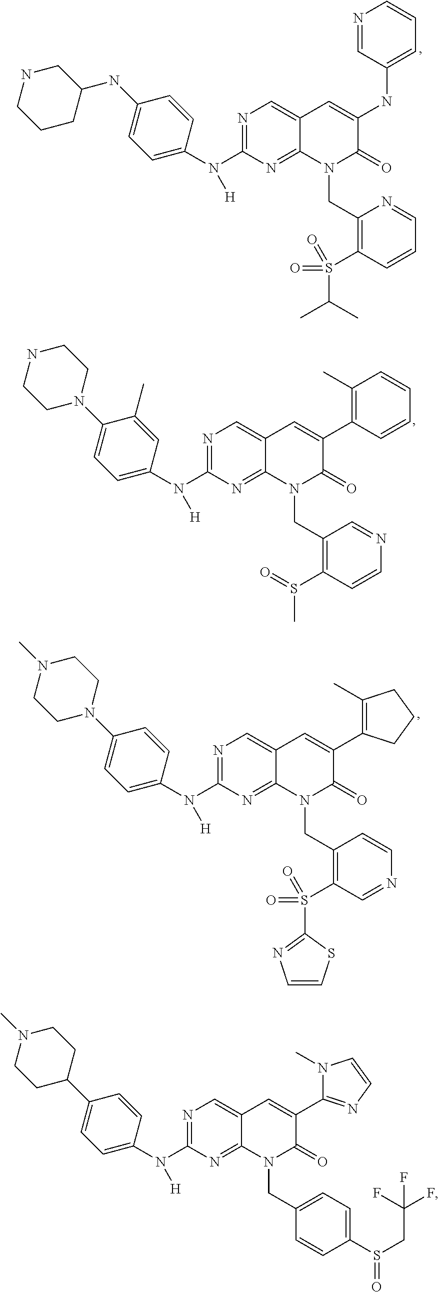 [SMILES: CC(C)S(=O)(=O)c1cccnc1Cn1c(=O)c(Nc2cccnc2)cc2cnc(Nc3ccc(NC4CCCNC4)cc3)nc21 ; Cc1ccccc1-c1cc2cnc(Nc3ccc(N4CCNCC4)c(C)c3)nc2n(Cc2cnccc2S(C)=O)c1=O ; CC1=C(c2cc3cnc(Nc4ccc(N5CCN(C)CC5)cc4)nc3n(Cc3ccncc3S(=O)(=O)c3nccs3)c2=O)CCC1 ; CN1CCC(c2ccc(Nc3ncc4cc(-c5nccn5C)c(=O)n(Cc5ccc(S(=O)CC(F)(F)F)cc5)c4n3)cc2)CC1]

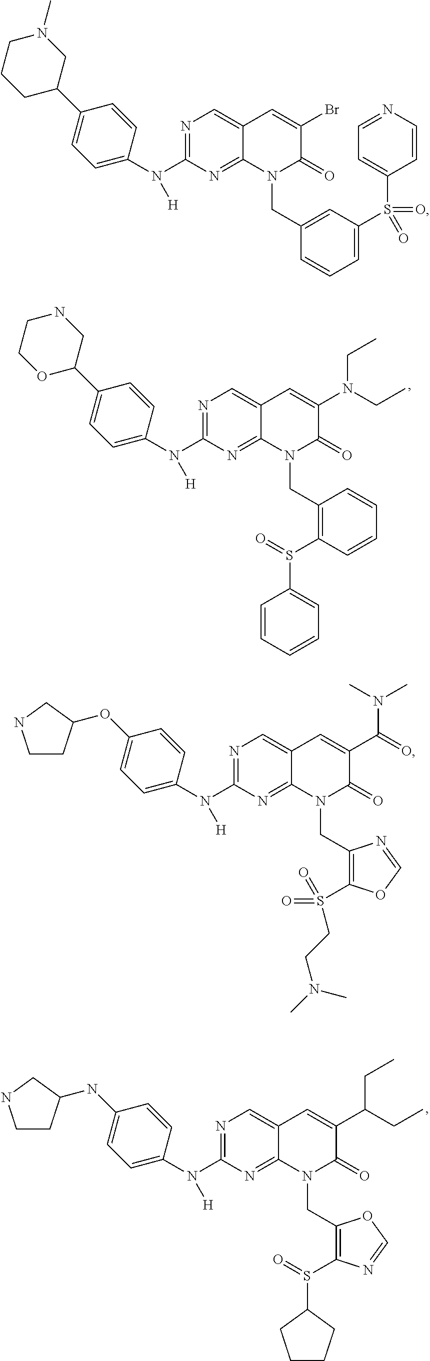 [SMILES: CN1CCCC(c2ccc(Nc3ncc4cc(Br)c(=O)n(Cc5cccc(S(=O)(=O)c6ccncc6)c5)c4n3)cc2)C1 ; CCN(CC)c1cc2cnc(Nc3ccc(C4CNCCO4)cc3)nc2n(Cc2ccccc2S(=O)c2ccccc2)c1=O ; CN(C)CCS(=O)(=O)c1ocnc1Cn1c(=O)c(C(=O)N(C)C)cc2cnc(Nc3ccc(OC4CCNC4)cc3)nc21 ; CCC(CC)c1cc2cnc(Nc3ccc(NC4CCNC4)cc3)nc2n(Cc2ocnc2S(=O)C2CCCC2)c1=O]

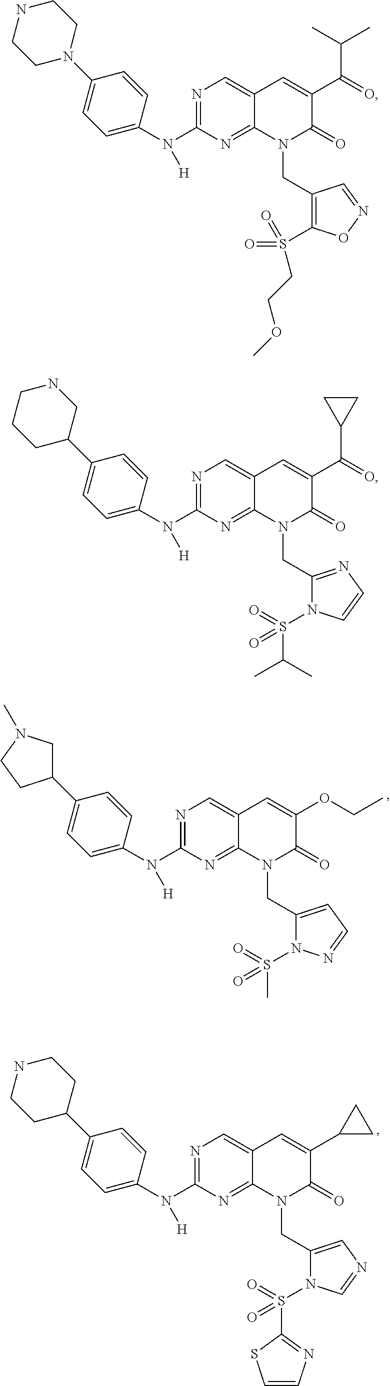 [SMILES: COCCS(=O)(=O)c1oncc1Cn1c(=O)c(C(=O)C(C)C)cc2cnc(Nc3ccc(N4CCNCC4)cc3)nc21 ; CC(C)S(=O)(=O)n1ccnc1Cn1c(=O)c(C(=O)C2CC2)cc2cnc(Nc3ccc(C4CCCNC4)cc3)nc21 ; CCOc1cc2cnc(Nc3ccc(C4CCN(C)C4)cc3)nc2n(Cc2ccnn2S(C)(=O)=O)c1=O ; O=c1c(C2CC2)cc2cnc(Nc3ccc(C4CCNCC4)cc3)nc2n1Cc1cncn1S(=O)(=O)c1nccs1]

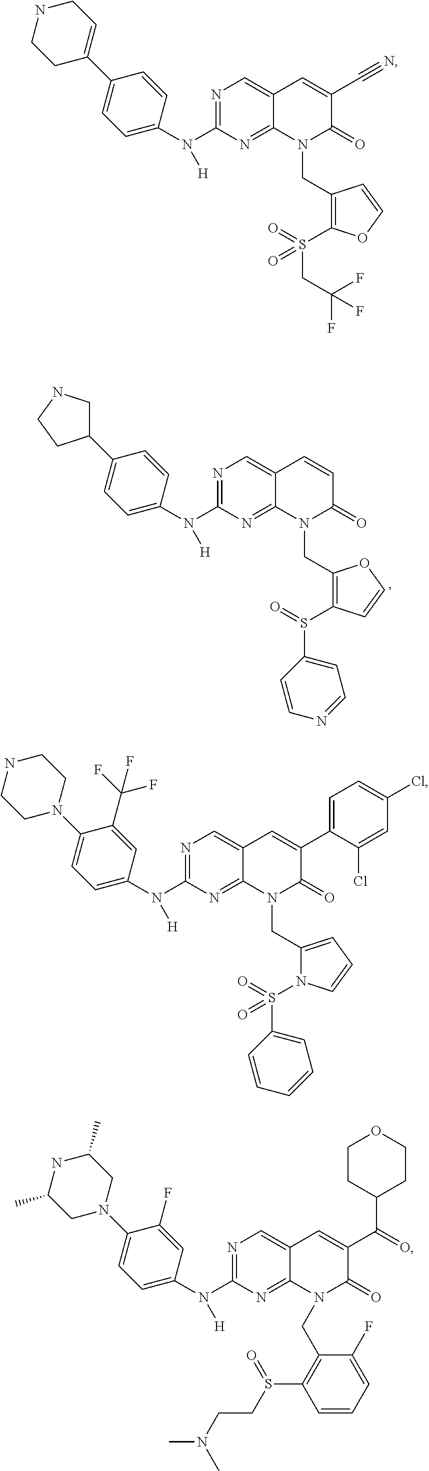 [SMILES: N#Cc1cc2cnc(Nc3ccc(C4=CCNCC4)cc3)nc2n(Cc2ccoc2S(=O)(=O)CC(F)(F)F)c1=O ; O=c1ccc2cnc(Nc3ccc(C4CCNC4)cc3)nc2n1Cc1occc1S(=O)c1ccncc1 ; O=c1c(-c2ccc(Cl)cc2Cl)cc2cnc(Nc3ccc(N4CCNCC4)c(C(F)(F)F)c3)nc2n1Cc1cccn1S(=O)(=O)c1ccccc1 ; C[C@@H]1CN(c2ccc(Nc3ncc4cc(C(=O)C5CCOCC5)c(=O)n(Cc5c(F)cccc5S(=O)CCN(C)C)c4n3)cc2F)C[C@H](C)N1]

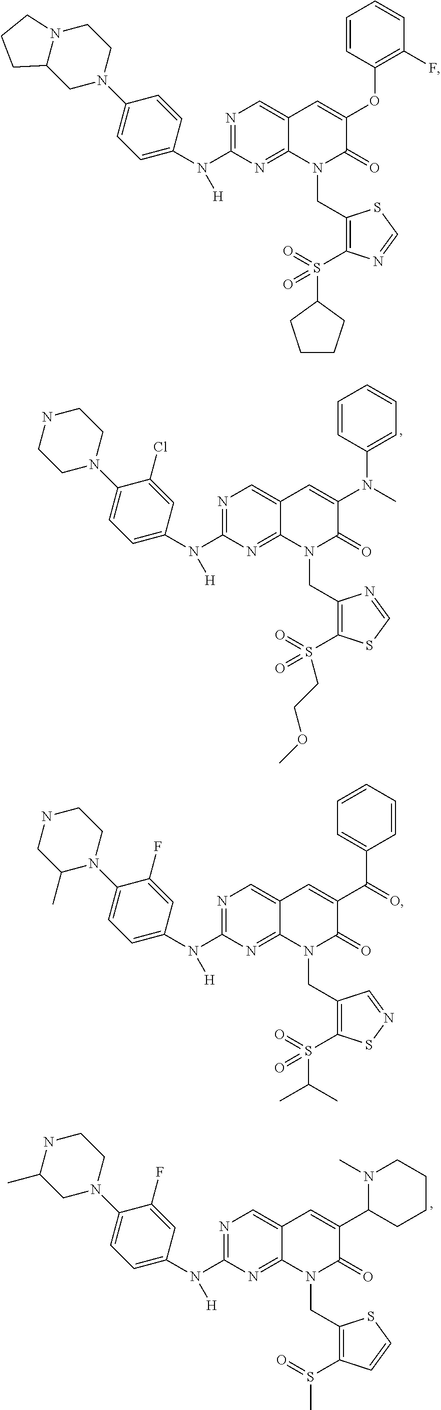

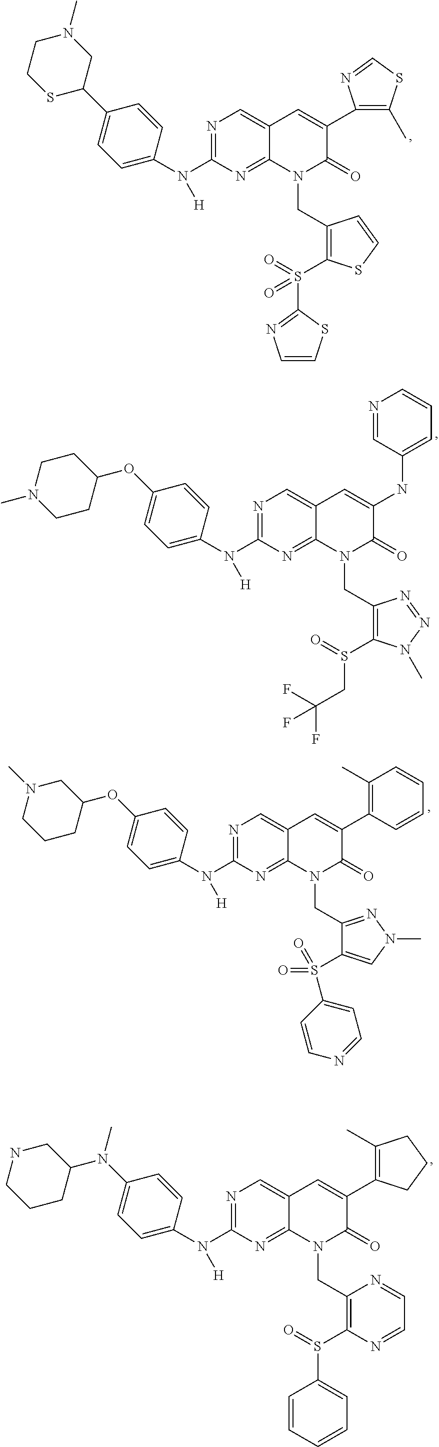

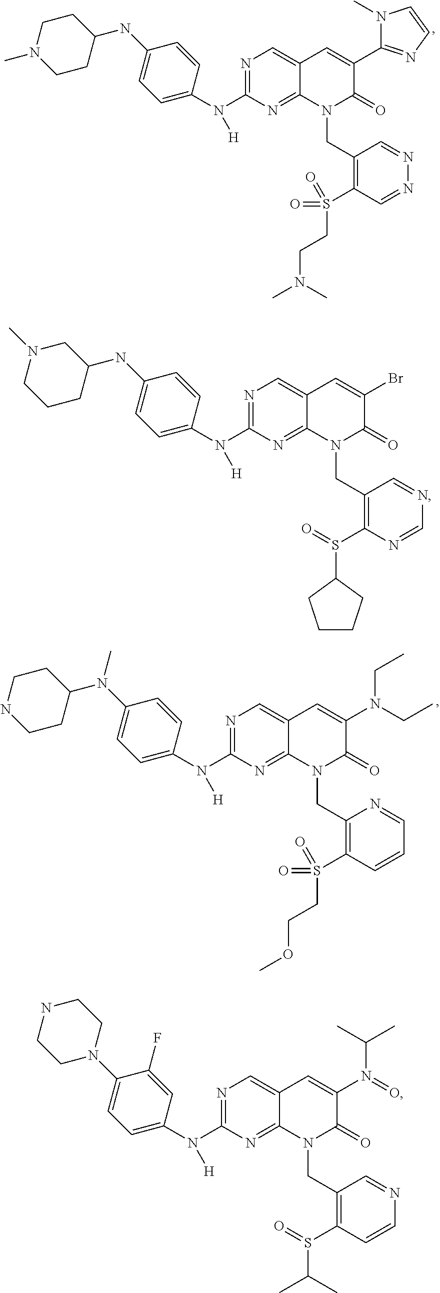

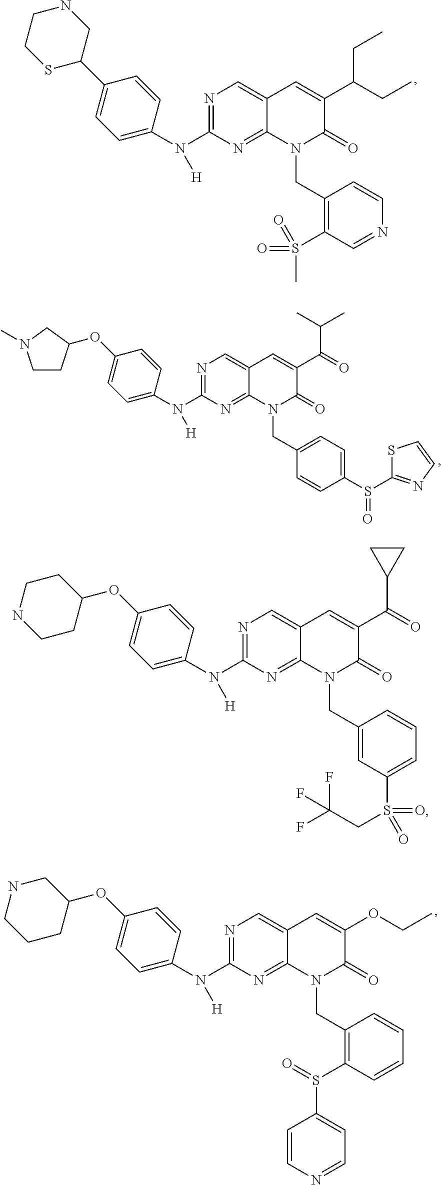

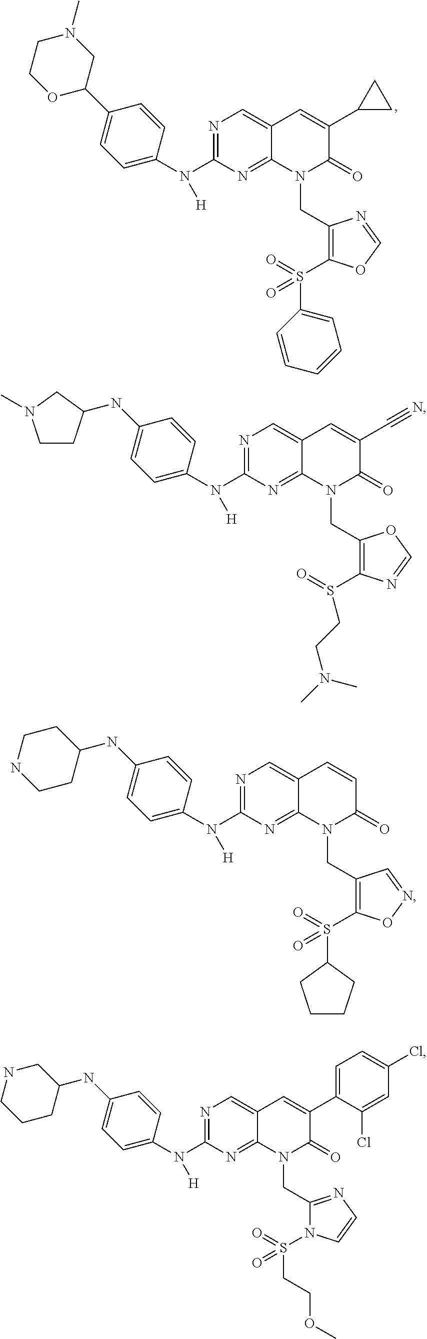

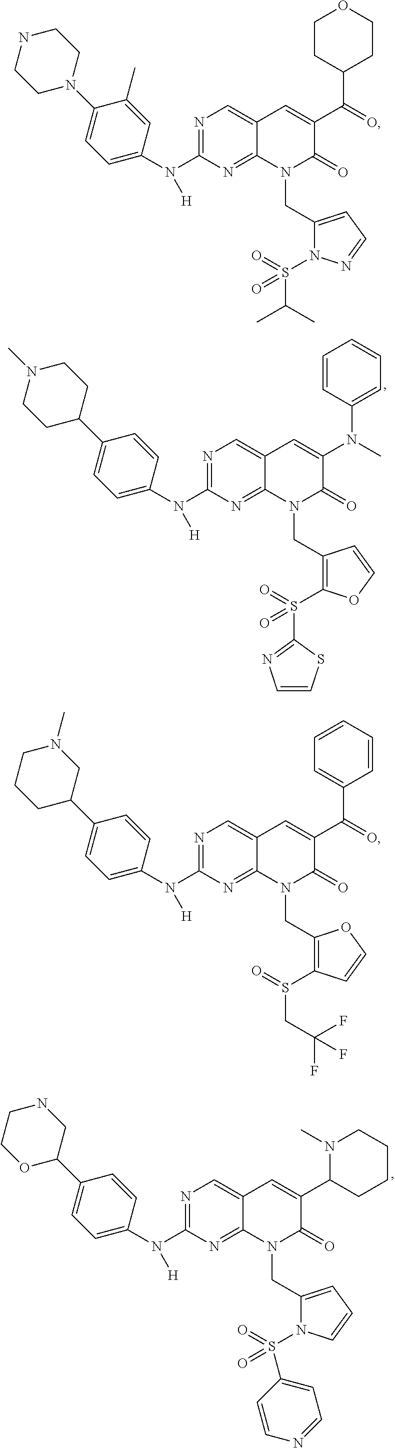

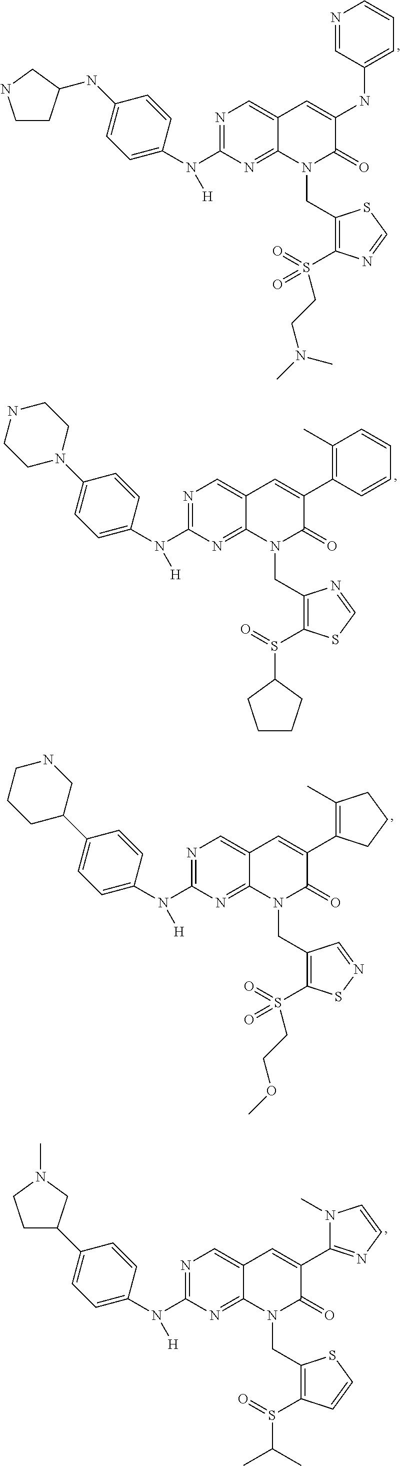 [SMILES: CN(C)CCS(=O)(=O)c1ncsc1Cn1c(=O)c(Nc2cccnc2)cc2cnc(Nc3ccc(NC4CCNC4)cc3)nc21 ; Cc1ccccc1-c1cc2cnc(Nc3ccc(N4CCNCC4)cc3)nc2n(Cc2ncsc2S(=O)C2CCCC2)c1=O ; COCCS(=O)(=O)c1sncc1Cn1c(=O)c(C2=C(C)CCC2)cc2cnc(Nc3ccc(C4CCCNC4)cc3)nc21 ; CC(C)S(=O)c1ccsc1Cn1c(=O)c(-c2nccn2C)cc2cnc(Nc3ccc(C4CCN(C)C4)cc3)nc21]

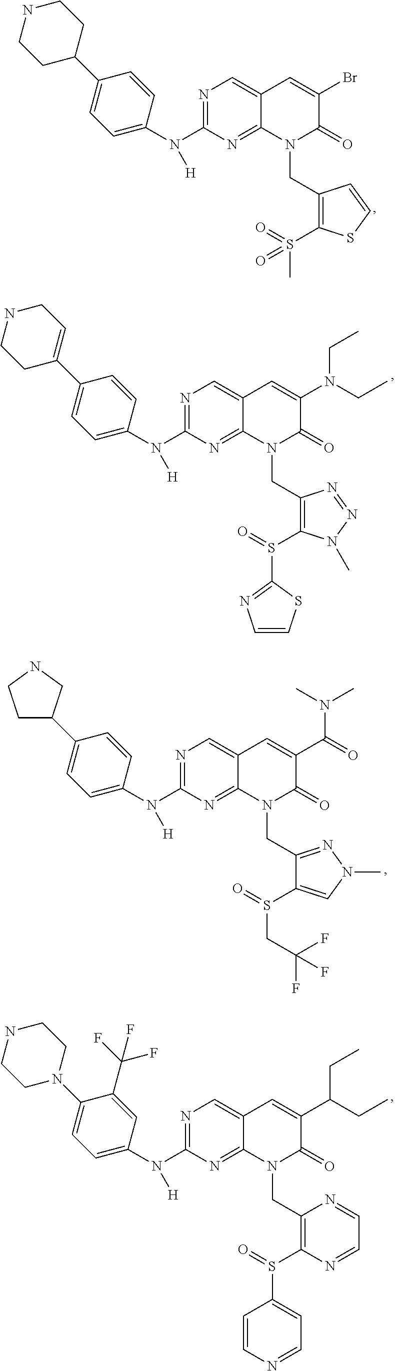 [SMILES: CS(=O)(=O)c1sccc1Cn1c(=O)c(Br)cc2cnc(Nc3ccc(C4CCNCC4)cc3)nc21 ; CCN(CC)c1cc2cnc(Nc3ccc(C4=CCNCC4)cc3)nc2n(Cc2nnn(C)c2S(=O)c2nccs2)c1=O ; CN(C)C(=O)c1cc2cnc(Nc3ccc(C4CCNC4)cc3)nc2n(Cc2nn(C)cc2S(=O)CC(F)(F)F)c1=O ; CCC(CC)c1cc2cnc(Nc3ccc(N4CCNCC4)c(C(F)(F)F)c3)nc2n(Cc2nccnc2S(=O)c2ccncc2)c1=O]

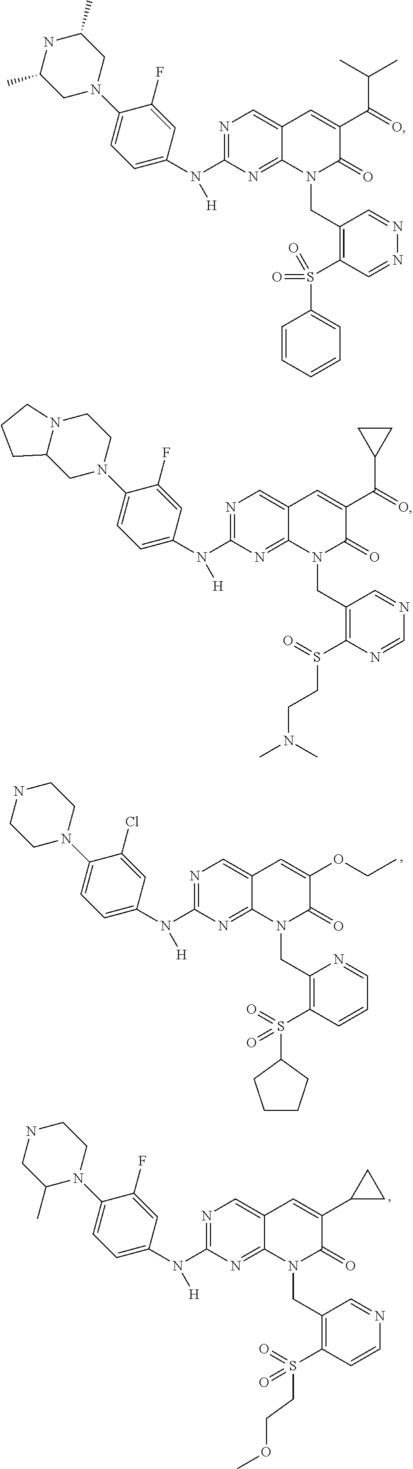 [SMILES: CC(C)C(=O)c1cc2cnc(Nc3ccc(N4C[C@@H](C)N[C@@H](C)C4)c(F)c3)nc2n(Cc2cnncc2S(=O)(=O)c2ccccc2)c1=O ; CN(C)CCS(=O)c1ncncc1Cn1c(=O)c(C(=O)C2CC2)cc2cnc(Nc3ccc(N4CCN5CCCC5C4)c(F)c3)nc21 ; CCOc1cc2cnc(Nc3ccc(N4CCNCC4)c(Cl)c3)nc2n(Cc2ncccc2S(=O)(=O)C2CCCC2)c1=O ; COCCS(=O)(=O)c1ccncc1Cn1c(=O)c(C2CC2)cc2cnc(Nc3ccc(N4CCNCC4C)c(F)c3)nc21]

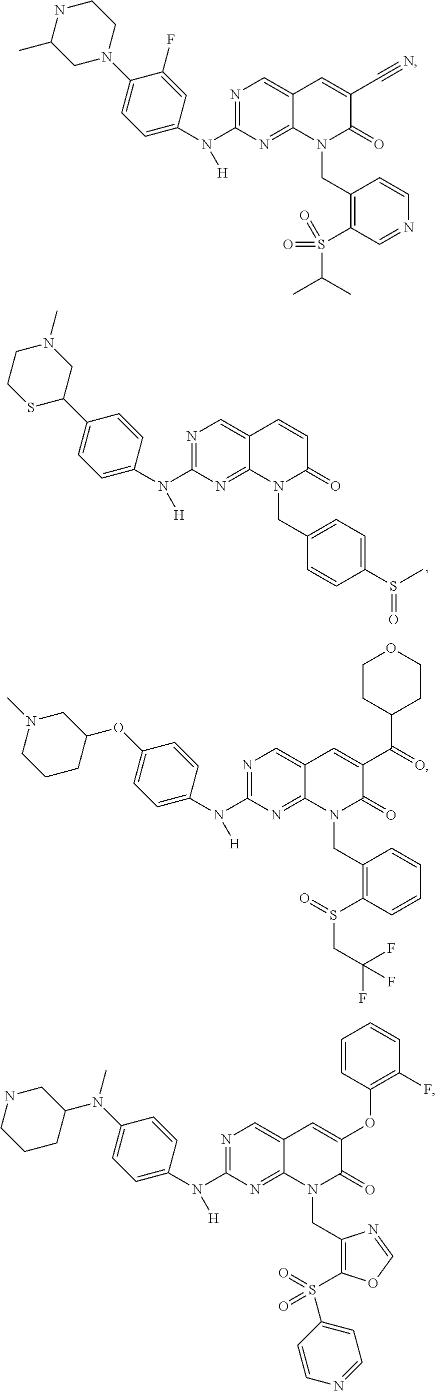

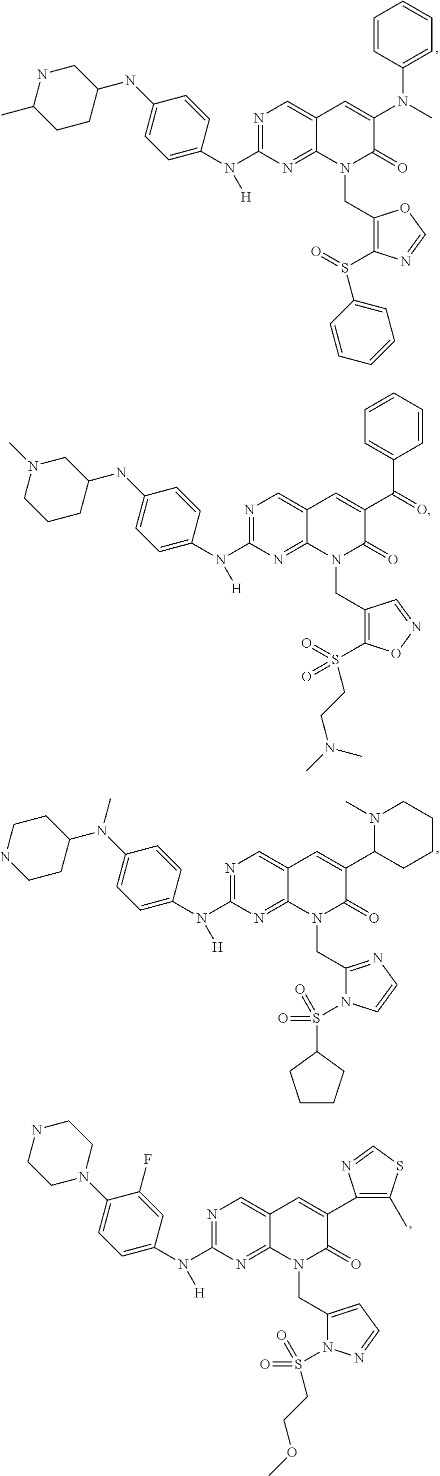

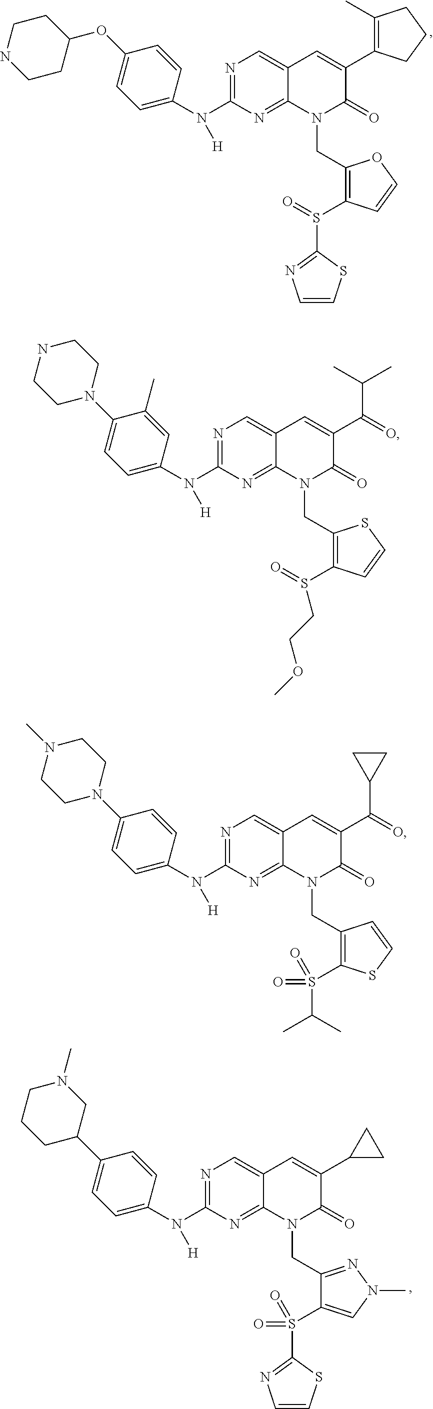 [SMILES: CC1=C(c2cc3cnc(Nc4ccc(OC5CCNCC5)cc4)nc3n(Cc3occc3S(=O)c3nccs3)c2=O)CCC1 ; COCCS(=O)c1ccsc1Cn1c(=O)c(C(=O)C(C)C)cc2cnc(Nc3ccc(N4CCNCC4)c(C)c3)nc21 ; CC(C)S(=O)(=O)c1sccc1Cn1c(=O)c(C(=O)C2CC2)cc2cnc(Nc3ccc(N4CCN(C)CC4)cc3)nc21 ; CN1CCCC(c2ccc(Nc3ncc4cc(C5CC5)c(=O)n(Cc5nn(C)cc5S(=O)(=O)c5nccs5)c4n3)cc2)C1]

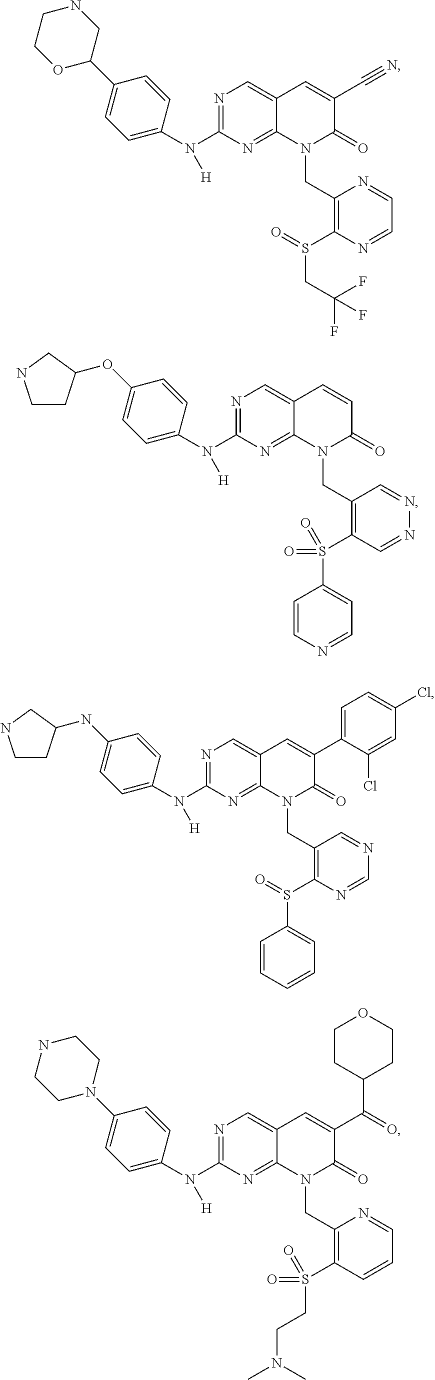 [SMILES: N#Cc1cc2cnc(Nc3ccc(C4CNCCO4)cc3)nc2n(Cc2nccnc2S(=O)CC(F)(F)F)c1=O ; O=c1ccc2cnc(Nc3ccc(OC4CCNC4)cc3)nc2n1Cc1cnncc1S(=O)(=O)c1ccncc1 ; O=c1c(-c2ccc(Cl)cc2Cl)cc2cnc(Nc3ccc(NC4CCNC4)cc3)nc2n1Cc1cncnc1S(=O)c1ccccc1 ; CN(C)CCS(=O)(=O)c1cccnc1Cn1c(=O)c(C(=O)C2CCOCC2)cc2cnc(Nc3ccc(N4CCNCC4)cc3)nc21]

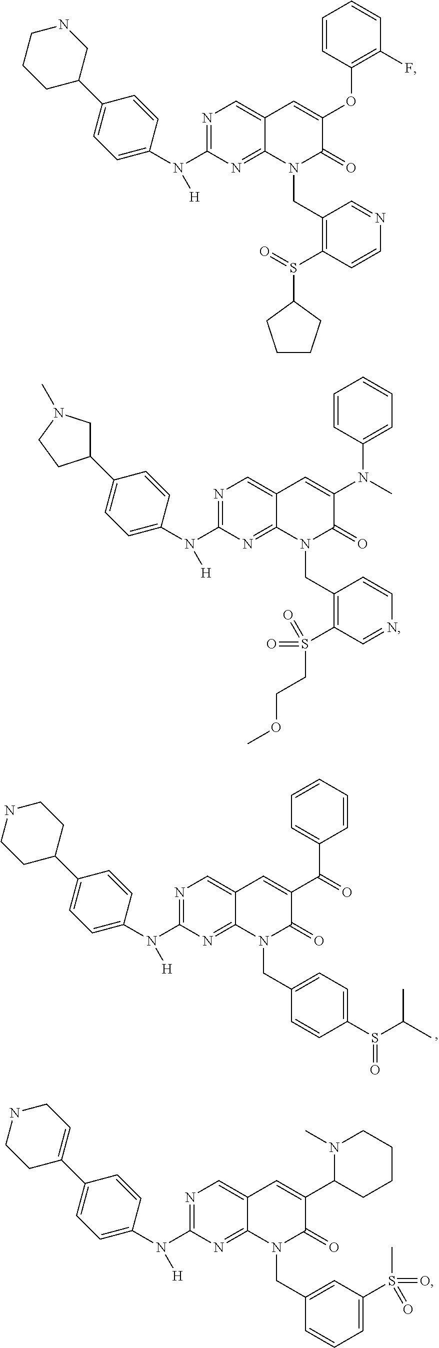 [SMILES: O=c1c(Oc2ccccc2F)cc2cnc(Nc3ccc(C4CCCNC4)cc3)nc2n1Cc1cnccc1S(=O)C1CCCC1 ; COCCS(=O)(=O)c1cnccc1Cn1c(=O)c(N(C)c2ccccc2)cc2cnc(Nc3ccc(C4CCN(C)C4)cc3)nc21 ; CC(C)S(=O)c1ccc(Cn2c(=O)c(C(=O)c3ccccc3)cc3cnc(Nc4ccc(C5CCNCC5)cc4)nc32)cc1 ; CN1CCCCC1c1cc2cnc(Nc3ccc(C4=CCNCC4)cc3)nc2n(Cc2cccc(S(C)(=O)=O)c2)c1=O]

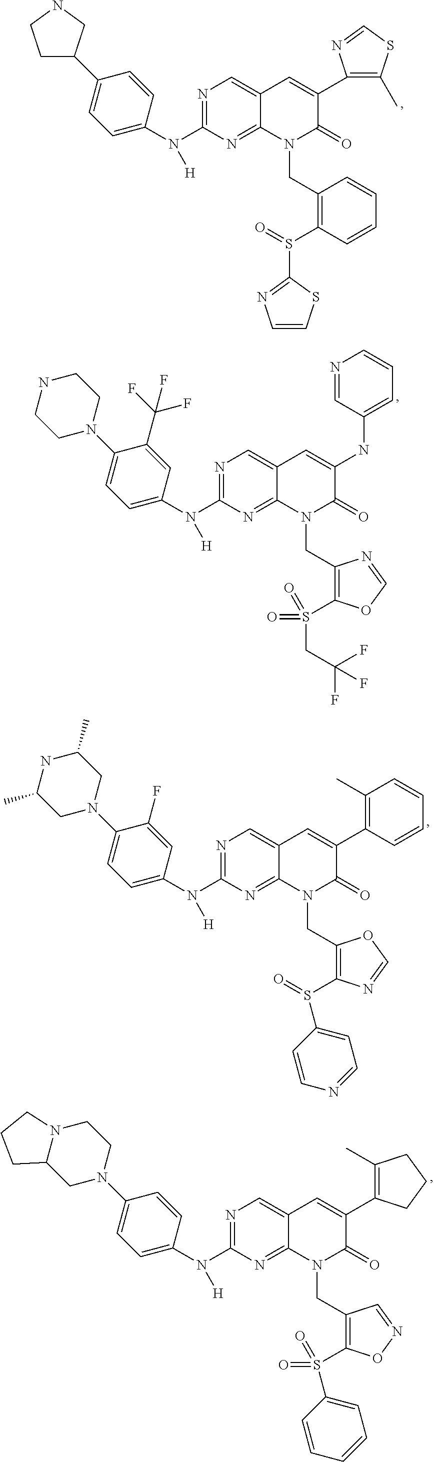

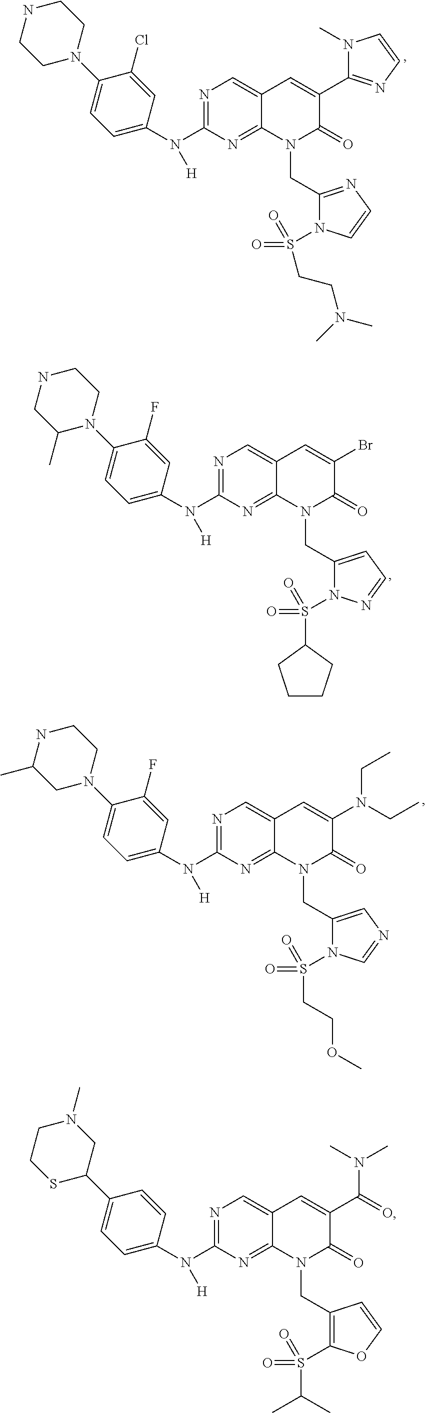 [SMILES: CN(C)CCS(=O)(=O)n1ccnc1Cn1c(=O)c(-c2nccn2C)cc2cnc(Nc3ccc(N4CCNCC4)c(Cl)c3)nc21 ; CC1CNCCN1c1ccc(Nc2ncc3cc(Br)c(=O)n(Cc4ccnn4S(=O)(=O)C4CCCC4)c3n2)cc1F ; CCN(CC)c1cc2cnc(Nc3ccc(N4CCNC(C)C4)c(F)c3)nc2n(Cc2cncn2S(=O)(=O)CCOC)c1=O ; CC(C)S(=O)(=O)c1occc1Cn1c(=O)c(C(=O)N(C)C)cc2cnc(Nc3ccc(C4CN(C)CCS4)cc3)nc21]

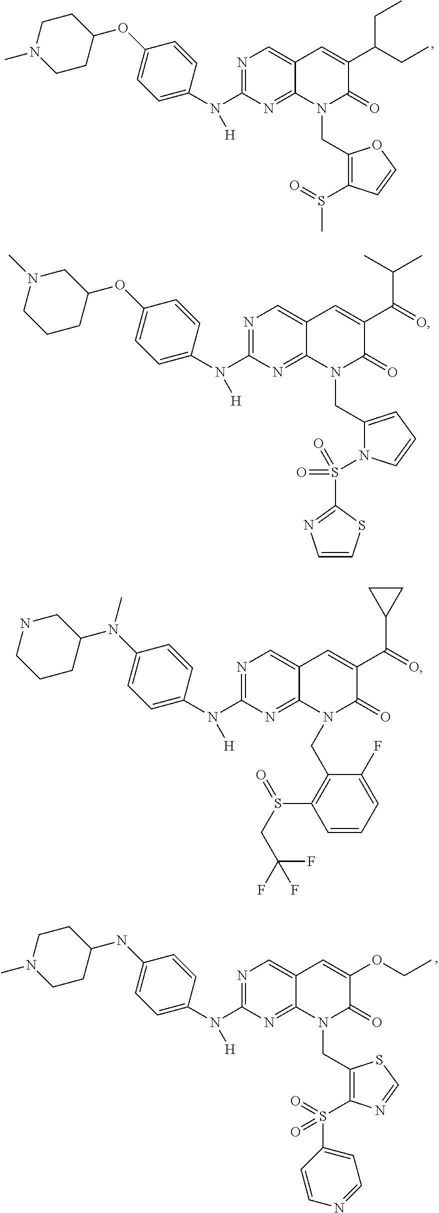

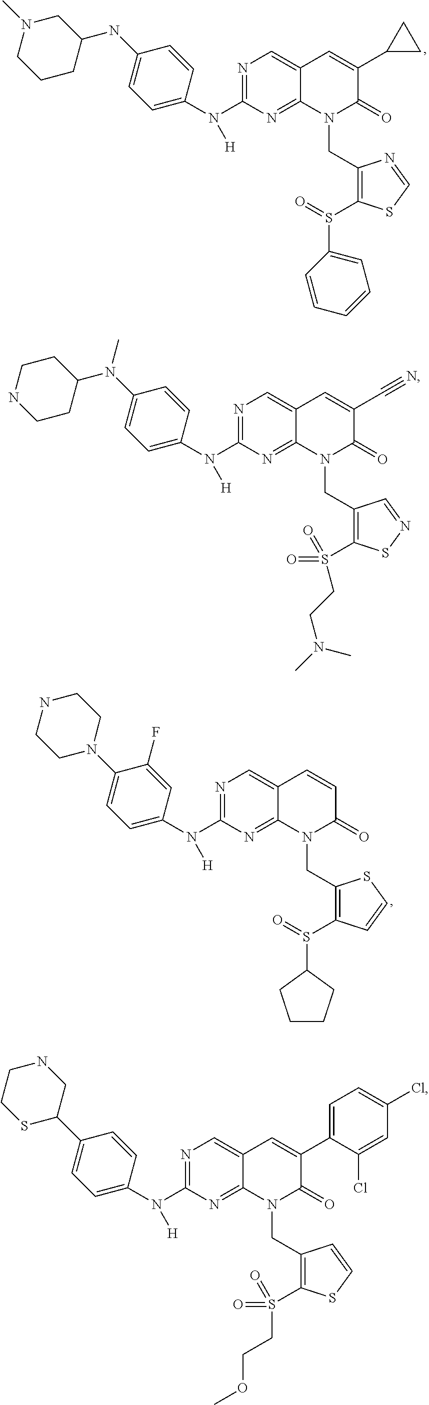 [SMILES: CN1CCCC(Nc2ccc(Nc3ncc4cc(C5CC5)c(=O)n(Cc5ncsc5S(=O)c5ccccc5)c4n3)cc2)C1 ; CN(C)CCS(=O)(=O)c1sncc1Cn1c(=O)c(C#N)cc2cnc(Nc3ccc(N(C)C4CCNCC4)cc3)nc21 ; O=c1ccc2cnc(Nc3ccc(N4CCNCC4)c(F)c3)nc2n1Cc1sccc1S(=O)C1CCCC1 ; COCCS(=O)(=O)c1sccc1Cn1c(=O)c(-c2ccc(Cl)cc2Cl)cc2cnc(Nc3ccc(C4CNCCS4)cc3)nc21]

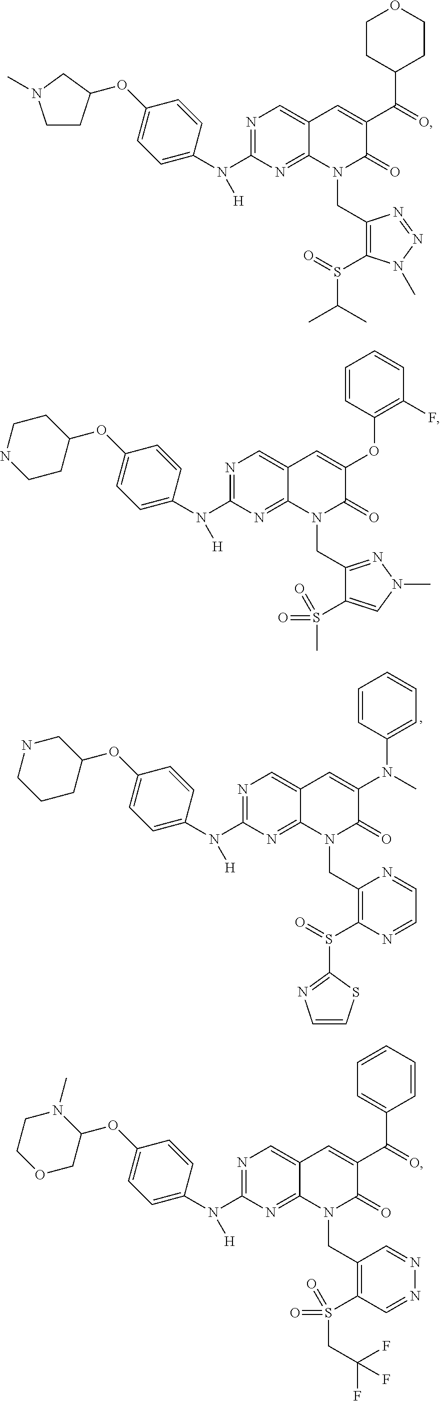

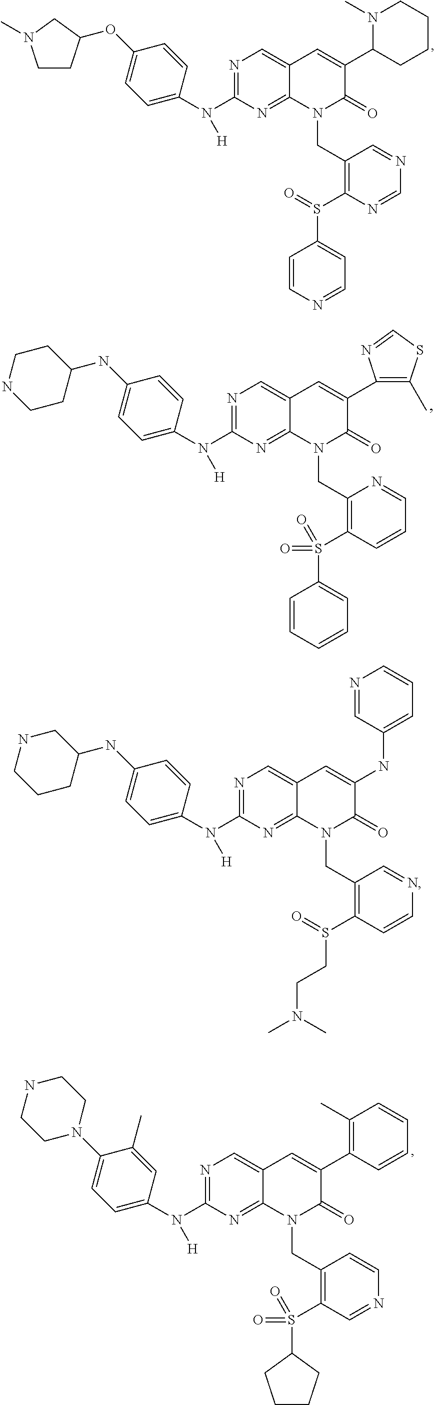 [SMILES: CN1CCC(Oc2ccc(Nc3ncc4cc(C5CCCCN5C)c(=O)n(Cc5cncnc5S(=O)c5ccncc5)c4n3)cc2)C1 ; Cc1scnc1-c1cc2cnc(Nc3ccc(NC4CCNCC4)cc3)nc2n(Cc2ncccc2S(=O)(=O)c2ccccc2)c1=O ; CN(C)CCS(=O)c1ccncc1Cn1c(=O)c(Nc2cccnc2)cc2cnc(Nc3ccc(NC4CCCNC4)cc3)nc21 ; Cc1ccccc1-c1cc2cnc(Nc3ccc(N4CCNCC4)c(C)c3)nc2n(Cc2ccncc2S(=O)(=O)C2CCCC2)c1=O]

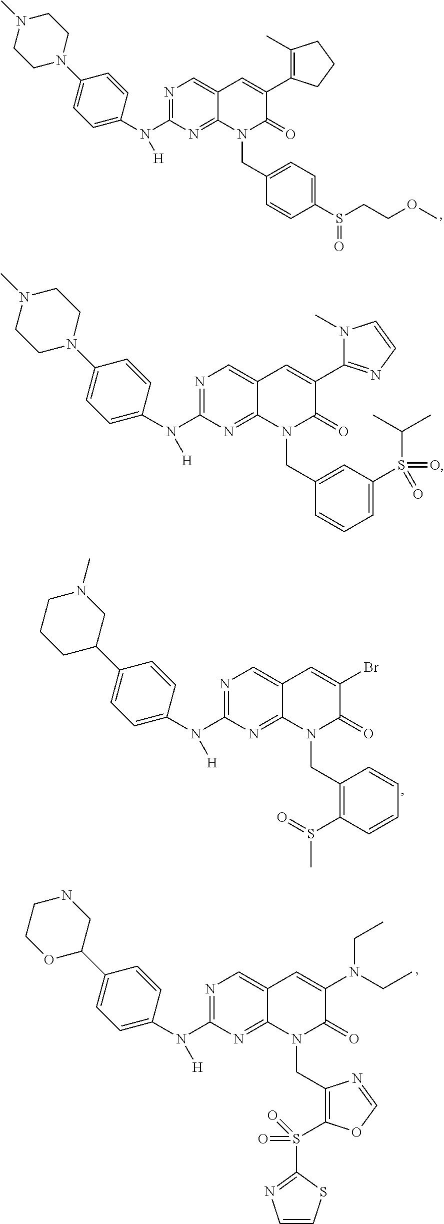

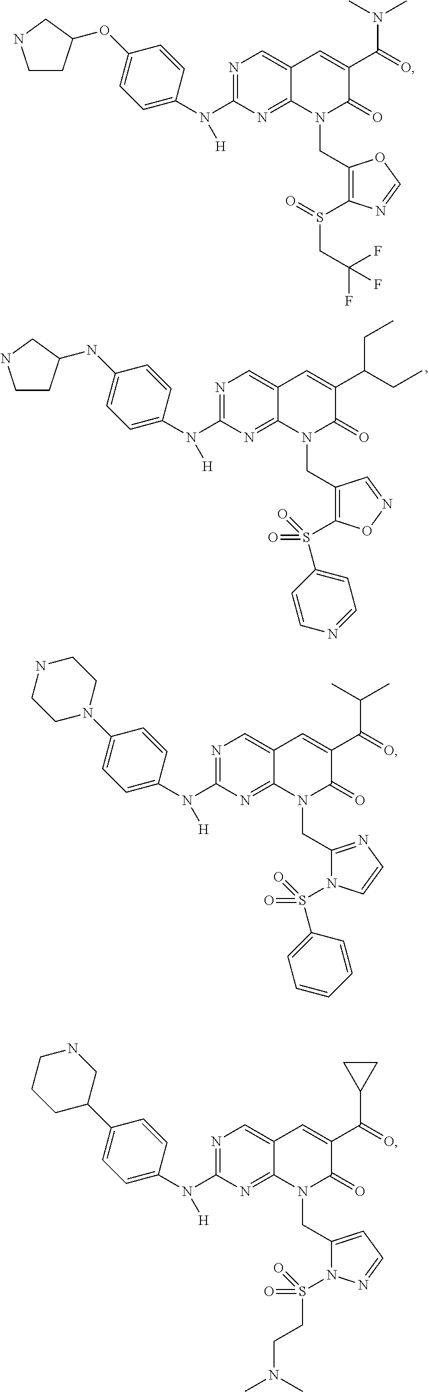

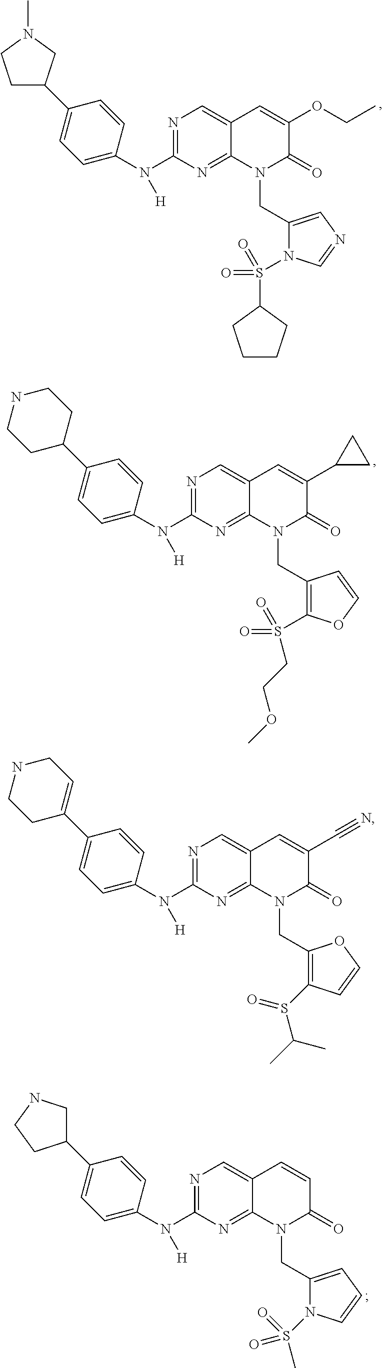 [SMILES: CCOc1cc2cnc(Nc3ccc(C4CCN(C)C4)cc3)nc2n(Cc2cncn2S(=O)(=O)C2CCCC2)c1=O ; COCCS(=O)(=O)c1occc1Cn1c(=O)c(C2CC2)cc2cnc(Nc3ccc(C4CCNCC4)cc3)nc21 ; CC(C)S(=O)c1ccoc1Cn1c(=O)c(C#N)cc2cnc(Nc3ccc(C4=CCNCC4)cc3)nc21 ; CS(=O)(=O)n1cccc1Cn1c(=O)ccc2cnc(Nc3ccc(C4CCNC4)cc3)nc21]

or a pharmaceutically acceptable salt, solvate or N-oxide thereof.

In another embodiment are PAK inhibitors suitable for treating cancer having the structure:

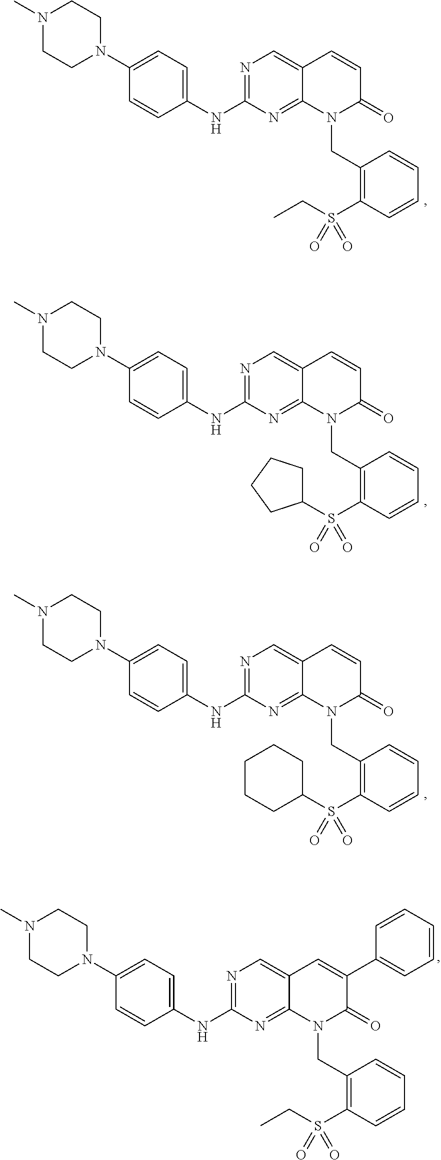 [SMILES: CCS(=O)(=O)c1ccccc1Cn1c(=O)ccc2cnc(Nc3ccc(N4CCN(C)CC4)cc3)nc21 ; CN1CCN(c2ccc(Nc3ncc4ccc(=O)n(Cc5ccccc5S(=O)(=O)C5CCCC5)c4n3)cc2)CC1 ; CN1CCN(c2ccc(Nc3ncc4ccc(=O)n(Cc5ccccc5S(=O)(=O)C5CCCCC5)c4n3)cc2)CC1 ; CCS(=O)(=O)c1ccccc1Cn1c(=O)c(-c2ccccc2)cc2cnc(Nc3ccc(N4CCN(C)CC4)cc3)nc21]

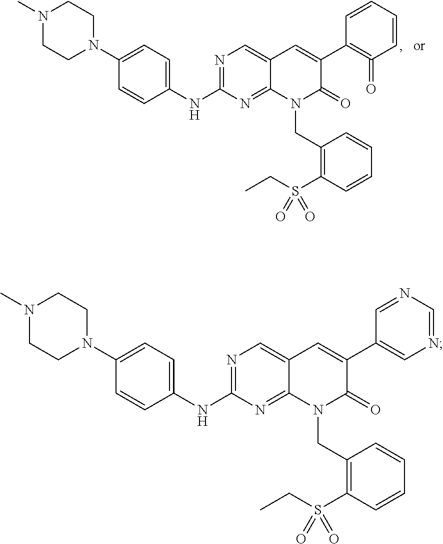

or a pharmaceutically acceptable salt, solvate or N-oxide thereof.

In another embodiment are PAK inhibitors suitable for treating cancer having the structure of Formula V or a pharmaceutically acceptable salt, solvate or N-oxide thereof wherein:

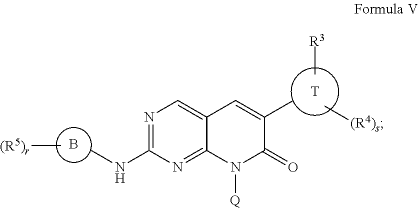

wherein:
ring T is aryl or heteroaryl;
R³ is —S(═O)R⁹, (R)—S(═O)R⁹, (S)—S(═O)R⁹, or —S(═O)₂R⁹;
each R⁴ is independently halogen, —CN, —NO₂, —OH, —OCF₃, —OCF₂H, —OCFH₂, —CF₃, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, or substituted or unsubstituted cycloalkyl;
R⁸ is H or R⁹;
R⁹ is substituted or unsubstituted alkyl, substituted or unsubstituted cycloalkyl, substituted or unsubstituted heterocycloalkyl, substituted or unsubstituted aryl, or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl, substituted or unsubstituted heteroaryl, or two R¹⁰ together with the atoms to which they are attached form a substituted or unsubstituted heterocycle;
s is 0-4;
Q is H, substituted or unsubstituted alkyl, substituted or unsubstituted cycloalkyl, substituted or unsubstituted heteroalkyl, substituted or unsubstituted heterocycloalkyl, substituted or unsubstituted cycloalkylalkyl, substituted or unsubstituted heterocycloalkylalkyl, substituted or unsubstituted aryl, substituted or unsubstituted arylalkyl, substituted or unsubstituted heteroaryl, or substituted or unsubstituted heteroarylalkyl;
ring B is aryl or heteroaryl;
each R⁵ is independently halogen, —CN, —NO₂, —OH, —OR¹⁰, —SR⁸, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl, or substituted or unsubstituted heterocycloalkyl; and
r is 0-8.

In certain embodiments, a compound of Formula V has the structure of Formula Va:

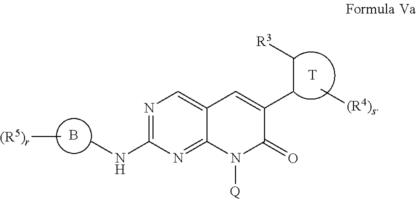

In some embodiments, a compound of Formula V has the structure of Formula Vb:

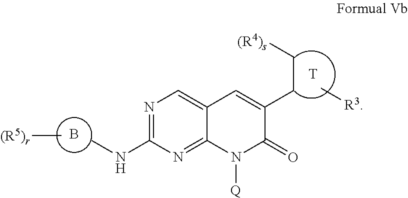

In certain embodiments, a compound of Formula V has the structure of Formula Vc or Formula Vd:

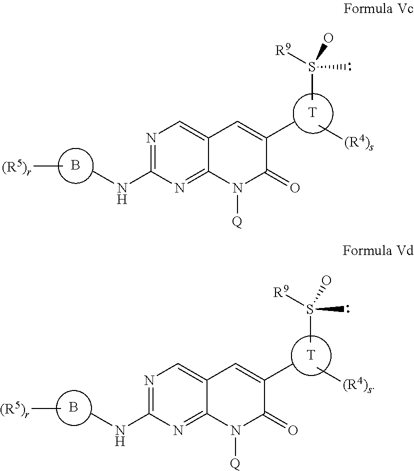

In some embodiments, a compound of Formula V has the structure of Formula Ve:

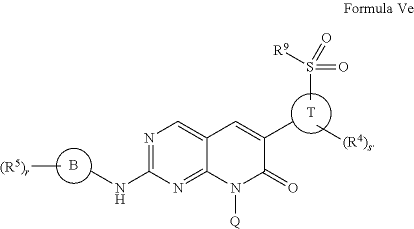

In certain embodiments, a compound of Formula V has the structure of Formula Vf:

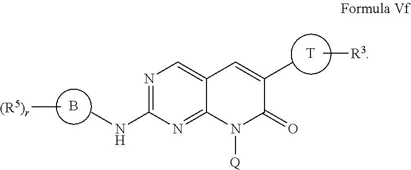

In one embodiment is a compound of any one of Formulae V-Vf, wherein ring T is a 5-10 membered heteroaryl ring comprising 0-4 nitrogen atoms, 0-2 oxygen atoms, 0-2 sulfur atoms, or any combination thereof, wherein at least one nitrogen, oxygen, or sulfur is present.

In a further embodiment is a compound of any one of Formulae V-Vf, wherein ring T is pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, pyrazine, or triazine. In another embodiment, ring T is pyridine. In yet another embodiment, ring T is pyrimidine. In a further embodiment, ring T is imidazole. In yet a further embodiment, ring T is pyrazole. In another embodiment, ring T is thiazole.

In yet another embodiment is a compound of any one of Formulae V-Vf, wherein ring T is aryl. In yet a further embodiment is a compound of any one of Formulae V-Vf, wherein ring T is phenyl.

In some embodiments, a compound of Formula V has the structure of Formula Vg:

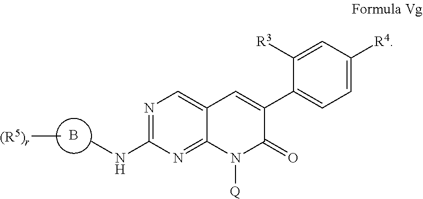

In certain embodiments, a compound of Formula V has the structure of Formula Vh:

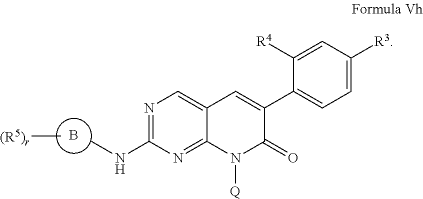

In some embodiments of a compound of any one of Formulae V-Vh, each R⁴ is independently halogen, —CN, —OH, —OCF₃, —CF₃, —SR⁸, —S(═O)R⁹, S(═O)₂R⁹, substituted or unsubstituted alkyl, or substituted or unsubstituted alkoxy. In certain embodiments of a compound of any one of Formulae V-Vh, R⁴ is halogen, methyl, or trifluoromethyl. In specific embodiments of a compound of Formula Vb, R⁴ is halogen, methyl, or trifluoromethyl. In one embodiment of a compound of any one of Formulae V-Vh, R⁴ is fluorine. In another embodiment, R⁴ is chlorine. In yet another embodiment, R⁴ is methyl. In a further embodiment, R⁴ is trifluoromethyl.

In certain embodiments of a compound of Formula V, Va, Vb, Ve, Vf, Vg, or Vh, ring T is selected from:

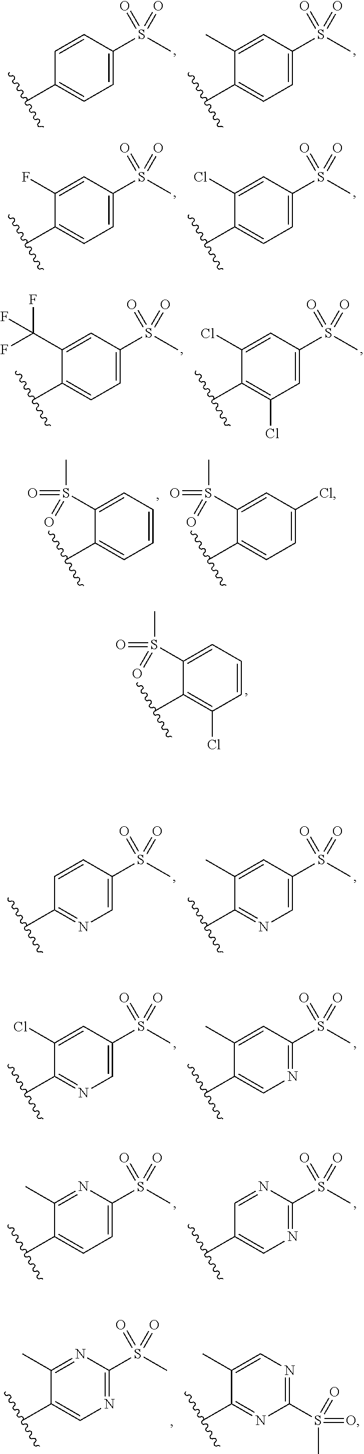

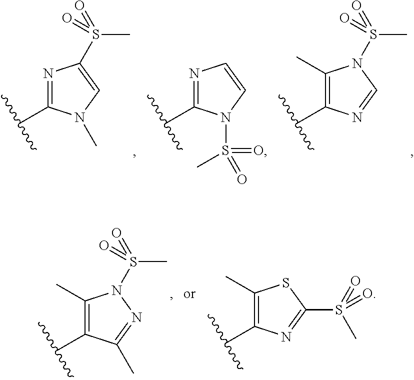

In further embodiments of a compound of Formula V, Va, Vb, Vc, Vd, Vf, Vg, or Vh, ring T is selected from:

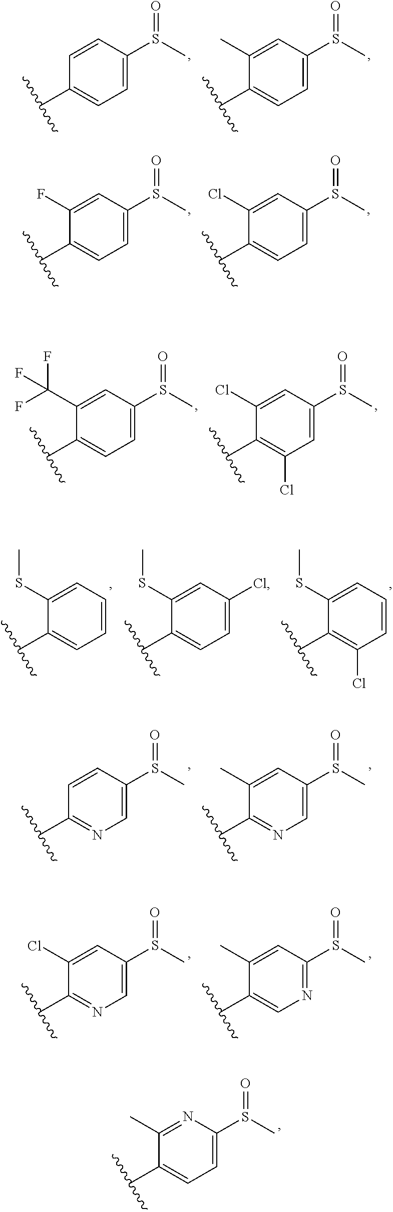

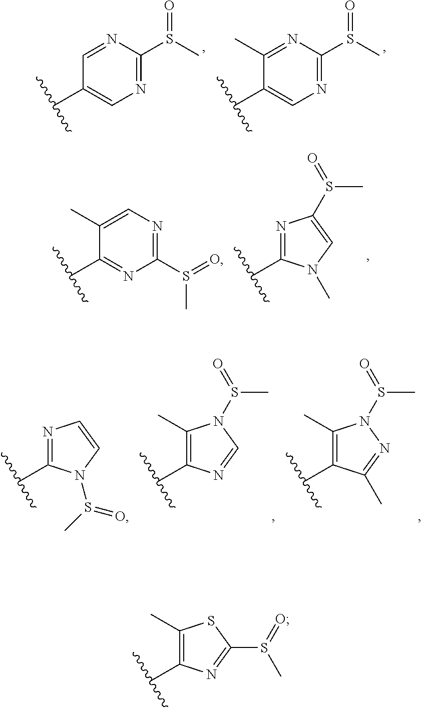

or the (S)- or (R)-enantiomers thereof.

In certain embodiments of a compound of any one of Formulae V-Vh, Q is substituted or unsubstituted aryl, substituted or unsubstituted heteroaryl, or substituted or unsubstituted arylalkyl. In some embodiments of a compound of any one of Formulae V-Vh, Q is substituted or unsubstituted benzyl. In specific embodiments of a compound of any one of Formulae V-Vh, Q is 2-chlorobenzyl, 2-trifluoromethylbenzyl, 2-fluorobenzyl, 2-cyanobenzyl, 2-methylbenzyl, 2-thiomethylbenzyl, 2-(F₃CS)benzyl, or 2-methoxybenzyl. In some embodiments of a compound of any one of Formulae V-Vh, Q is substituted or unsubstituted phenyl, substituted or unsubstituted pyridine, substituted or unsubstituted pyrimidine, or substituted or unsubstituted pyrazine. In one embodiment of a compound of Formula V-Vh, Q is phenyl. In one embodiment is a compound of Formula V-Vh wherein Q is H. In another embodiment, Q is not H. In another embodiment, Q is trifluormethylbenzyl. In yet another embodiment, Q is thiazole or alkylthiazole.

In some embodiments of a compound of any one of Formulae V-Vh, Q is substituted or unsubstituted alkyl, substituted or unsubstituted cycloalkyl, or substituted or unsubstituted heterocycloalkyl. In certain embodiments of a compound of any one of Formulae V-Vh, Q is methyl, ethyl, n-propyl, isopropyl, n-butyl, isobutyl, tert-butyl, cyclopropyl, cyclobutyl, cyclopentyl, cyclohexyl, morpholine, tetrahydropyran, piperidine, piperazine, or N-methyl piperazine. In one embodiment of a compound of Formula V-Vh, Q is tetrahydrofuran. In another embodiment, Q substituted or unsubstituted piperidine.

In certain embodiments of a compound of any one of Formulae V-Vh, Q is substituted or unsubstituted cycloalkylalkyl, or substituted or unsubstituted heterocycloalkylalkyl. In one embodiment of a compound of Formula V-Vh, Q is cyclopropylalkyl. In another embodiment, Q is tetrahydropyranylalkyl. In yet another embodiment, Q is morpholinoalkyl.

In certain embodiments of a compound of any one of Formulae V-Vh, Q is selected from:

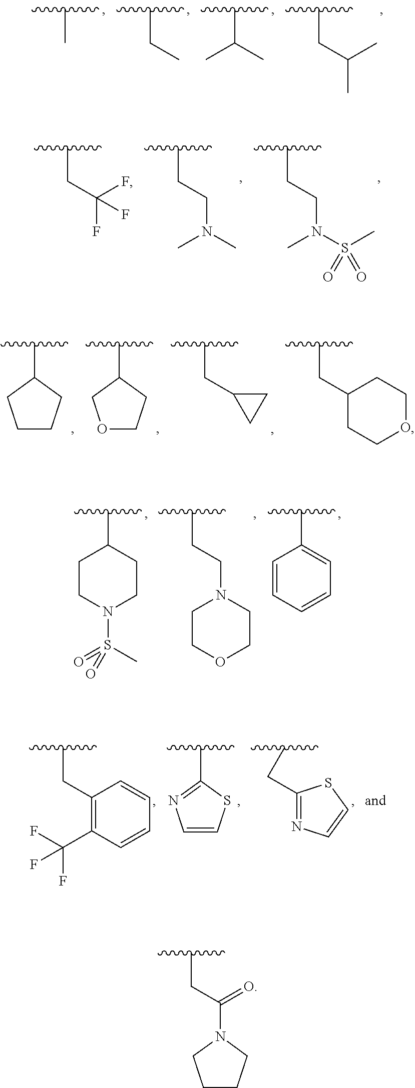

In certain embodiments of a compound of any one of Formulae V-Vh, ring B is selected from:

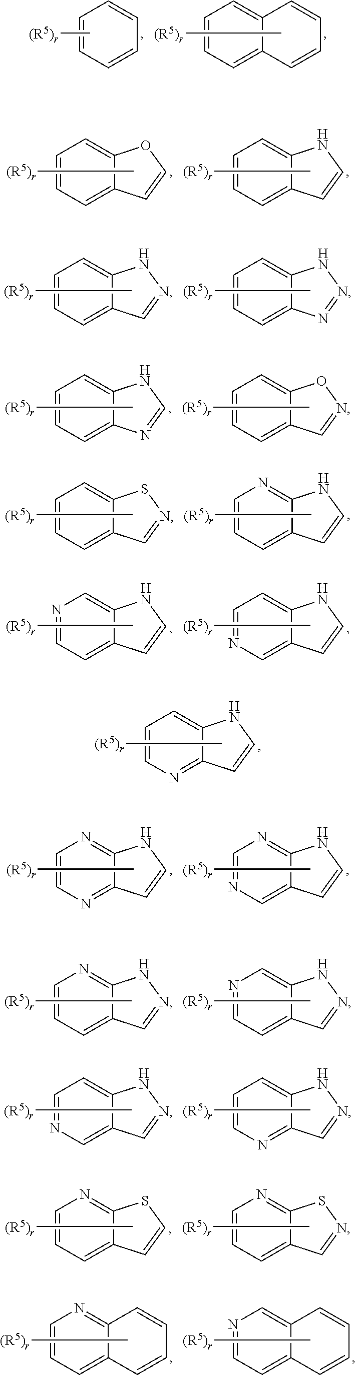

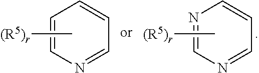

In some embodiments of a compound of any one of Formulae V-Vh, ring B is substituted or unsubstituted phenyl.

In some embodiments of a compound of any of Formulae V-Vh, R⁵ is H, halogen, —CN, —OH, —OR¹⁰, —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted alkoxy, or substituted or unsubstituted heterocycloalkyl.

In certain embodiments of a compound of any one of Formulae V-Vh, at least one R⁵ is halogen, —CN, —OH, —NR¹⁰S(═O)₂R⁹, —S(═O)₂N(R¹⁰)₂, —N(R¹⁰)₂, —C(═O)N(R¹⁰)₂, —NR¹⁰C(═)R¹⁰, —NR¹⁰C(═O)OR¹⁰, —NR¹⁰C(═O)N(R¹⁰)₂, substituted or unsubstituted alkyl, substituted or unsubstituted alkoxy, or substituted or unsubstituted heterocycloalkyl. In specific embodiments of a compound of any one of Formulae V-Vh, at least one R⁵ is halogen. In another embodiment, at least one R⁵ is fluorine. In yet another embodiment, at least one R⁵ is trifluoromethyl.

In some embodiments of a compound of any one of Formulae V-Vh, at least one R⁵ is —OR¹⁰, —N(R¹⁰)₂, —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In certain embodiments of a compound of any one of Formulae V-Vh, at least one R⁵ is substituted or unsubstituted heterocycloalkyl. In some embodiments of a compound of any of Formulae V-Vh, at least one R⁵ is a substituted or unsubstituted piperazine, substituted or unsubstituted piperidine, substituted or unsubstituted pyrrolidine, or substituted or unsubstituted morpholine. In certain embodiments of a compound of any one of Formulae V-Vh, at least one R⁵ is)-N(R¹⁰)₂. In some embodiments of a compound of any of, at least one R⁵ is —OR¹⁰.

In another embodiment, is a compound of Formula V-Vh wherein R⁵ is a C₃-C₆cycloalkyl ring. In another embodiment, R⁵ is cyclopropyl. In another embodiment, R⁵ is cyclopentyl. In another embodiment, R⁵ is cyclohexyl.

In another embodiment, is a compound of Formula V-Vh wherein R⁵ is a substituted or unsubstituted heterocycloalkyl ring. In another embodiment, R⁵ is a substituted or unsubstituted piperazine. In another embodiment, R⁵ is an unsubstituted piperazine. In another embodiment, R⁵ is N-methylpiperazine. In another embodiment, R⁵ is a substituted or unsubstituted piperidine. In another embodiment, R⁵ is an unsubstituted piperidine.

In another embodiment is a compound of Formula V-Vh wherein r is 0. In another embodiment, r is 1. In a further embodiment, r is 2. In another embodiment is a compound of Formula V-Vh wherein r is 1; and R⁵ is substituted or unsubstituted piperazine.

In one embodiment is a compound of Formula V-Vh, wherein ring B is substituted with) —N(R¹⁰)₂, wherein R¹⁰ is each independently selected from H and a substituted or unsubstituted heterocycloalkyl. In another embodiment is a compound of Formula V-Vh, wherein ring B is substituted with —NHR¹⁰, wherein R¹⁰ is a substituted or unsubstituted piperazine, substituted or unsubstituted piperidine, substituted or unsubstituted pyrrolidine or substituted or unsubstituted morpholine. In a further embodiment is a compound of Formula V-Vh, wherein ring B is substituted with —N(CH₃)R¹⁰, wherein R¹⁰ is a substituted or unsubstituted piperazine, substituted or unsubstituted piperidine, substituted or unsubstituted pyrrolidine or substituted or unsubstituted morpholine.

Also presented herein is a compound of Formula V-Vh, wherein ring B is substituted with —OR¹⁰, wherein R¹⁰ is a substituted or unsubstituted heterocycloalkyl. In another embodiment is a compound of Formula V-Vh, wherein ring B is substituted with —OR¹⁰ wherein R¹⁰ is a substituted or unsubstituted piperazine, substituted or unsubstituted piperidine, substituted or unsubstituted pyrrolidine or substituted or unsubstituted morpholine.

In yet another embodiment of a compound of Formula V-Vh, ring B is substituted with at least two R⁵. In another embodiment, ring B is substituted with halogen and a substituted or unsubstituted heterocycloalkyl. In a further embodiment, ring B is substituted with at least one F, Cl, Br, I, or trifluoromethyl and a substituted or unsubstituted piperazine, substituted or unsubstituted piperidine, substituted or unsubstituted pyrrolidine, or substituted or unsubstituted morpholine.

In one embodiment is a compound of Formula V-Vh wherein

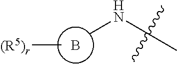

is selected from:

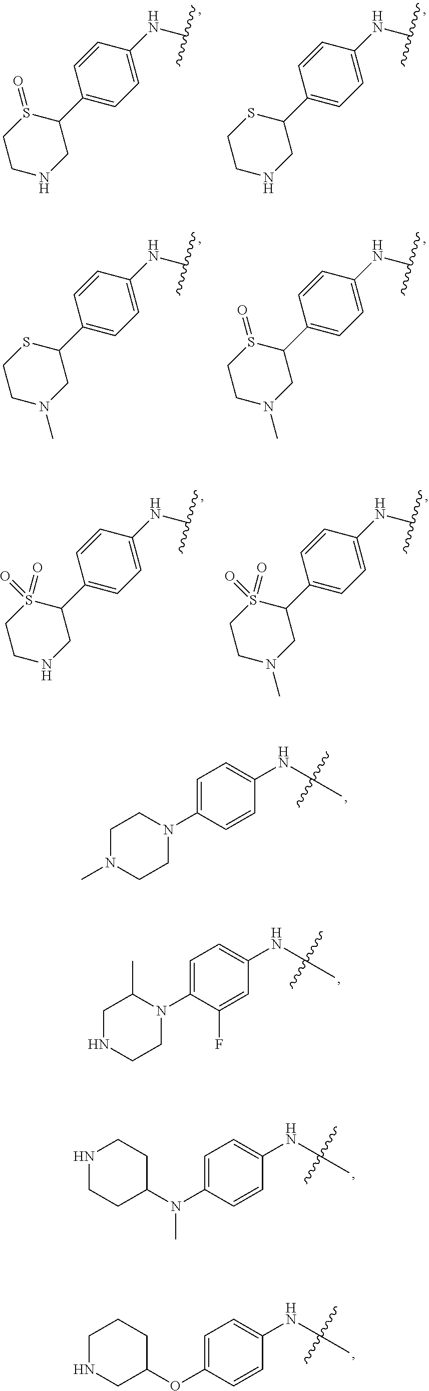

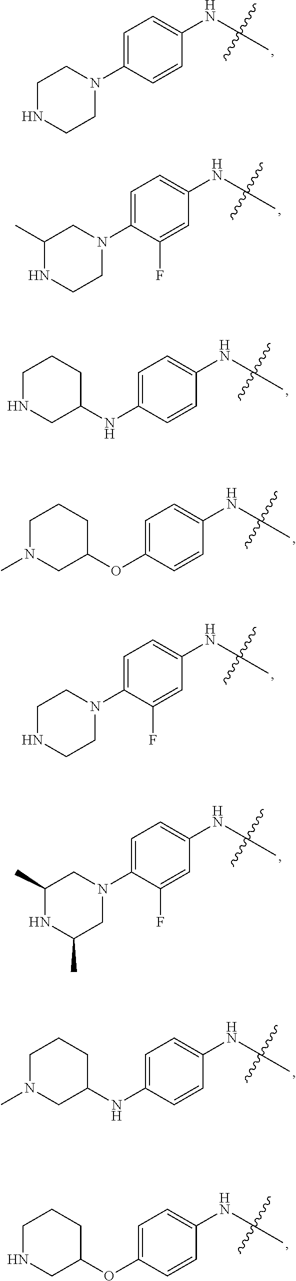

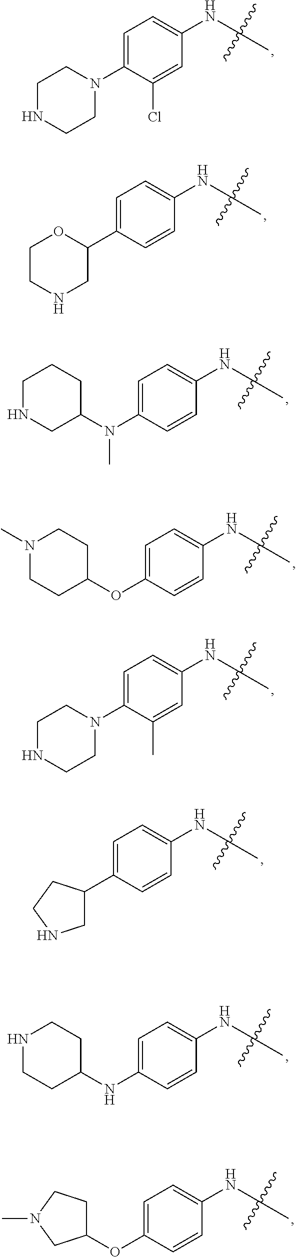

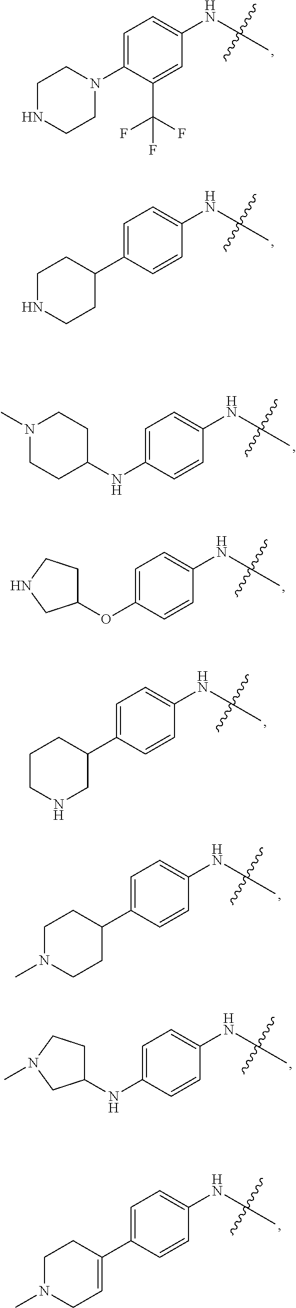

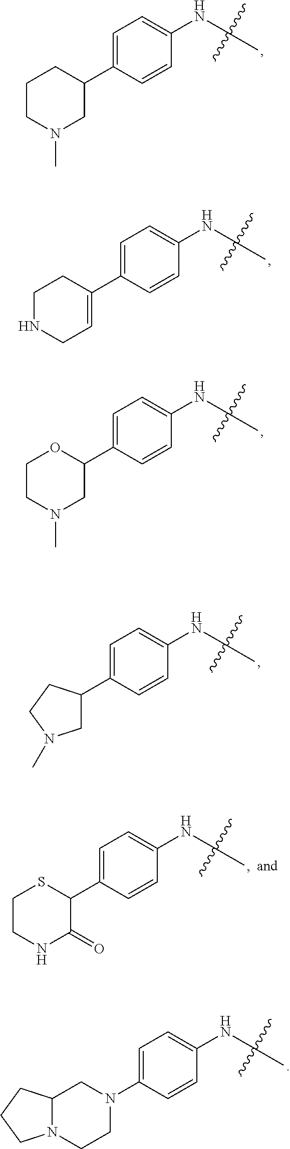 [SMILES: *Nc1ccc(C2CCCN(C)C2)cc1 ; *Nc1ccc(C2=CCNCC2)cc1 ; *Nc1ccc(C2CN(C)CCO2)cc1 ; *Nc1ccc(C2CCN(C)C2)cc1 ; *Nc1ccc(C2SCCNC2=O)cc1 ; *Nc1ccc(N2CCN3CCCC3C2)cc1]

In yet another embodiment is a compound of Formula V-Vh wherein

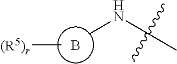

is selected from:

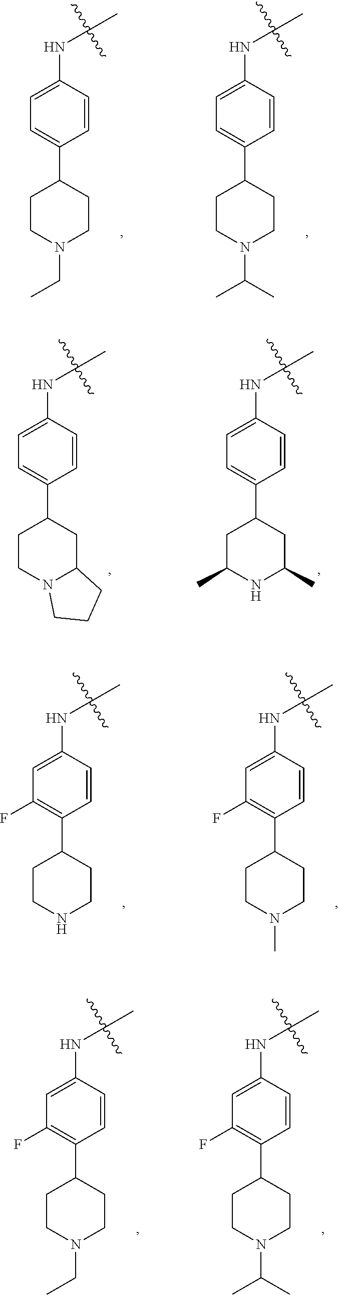

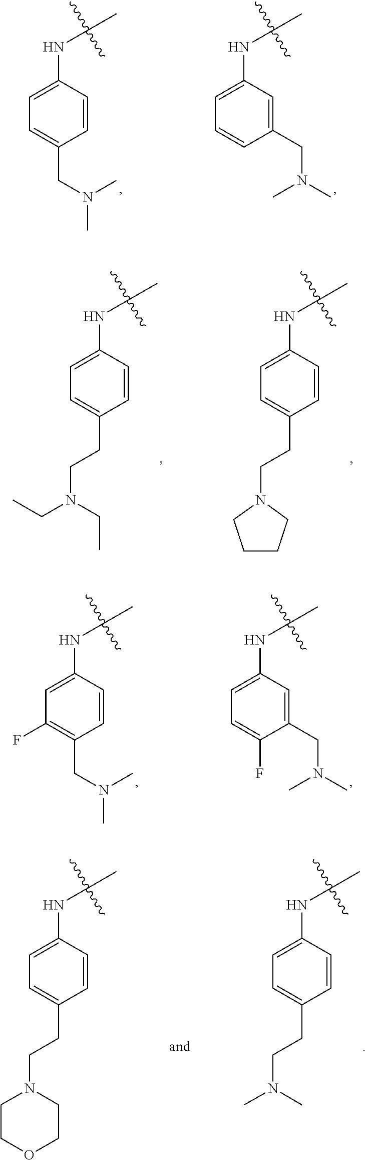

In one embodiment, is a compound of Formula V-Vh, wherein R⁵ is halogen, —CN, —OH, substituted or unsubstituted alkyl, —OR¹⁰, —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, NR¹⁰C(═O)N(R¹⁰)₂, or substituted or unsubstituted heterocycloalkyl. In one embodiment, R⁵ is selected from F, Cl, Br, or I. In another embodiment R⁵ is F.

In another embodiment, is a compound of Formula V-Vh, wherein at least one R⁵ is —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In one embodiment, is a compound of Formula V-Vh, wherein at least one R⁵ is)-N(R¹⁰)₂, or substituted or unsubstituted heterocycloalkyl. In yet another embodiment, is a compound of Formula V-Vh wherein at least one of R⁵ is a substituted or unsubstituted piperazine, substituted or unsubstituted piperidine, substituted or unsubstituted pyrrolidine or substituted or unsubstituted morpholine. In a further embodiment, is a compound of Formula V-Vh, wherein at least one R⁵ is —OR¹⁰. In one embodiment is a compound of Formula V-Vh, wherein at least one R⁵ is —OR¹⁰ and R¹⁰ is H. In another embodiment, R¹⁰ is alkyl selected from methyl, ethyl, n-propyl, iso-propyl, n-butyl, iso-butyl, and tert-butyl.

In one embodiment is a compound of Formula V-Vh wherein ring B is substituted with) —N(R¹⁰)₂, wherein R¹⁰ is each independently selected from H and a substituted or unsubstituted heterocycloalkyl. In another embodiment is a compound of Formula V-Vh wherein ring B is substituted with —NHR¹⁰ wherein R¹⁰ is a substituted or unsubstituted piperazine, substituted or unsubstituted piperidine, substituted or unsubstituted pyrrolidine or substituted or unsubstituted morpholine. In a further embodiment is a compound of Formula V-Vh wherein ring B is substituted with —N(CH₃)R¹⁰ wherein R¹⁰ is a substituted or unsubstituted piperazine, substituted or unsubstituted piperidine, substituted or unsubstituted pyrrolidine or substituted or unsubstituted morpholine.

Also presented herein is a compound of Formula V-Vh wherein ring B is substituted with —OR¹⁰ wherein R¹⁰ is a substituted or unsubstituted heterocycloalkyl. In another embodiment is a compound of Formula V-Vh wherein ring B is substituted with —OR¹⁰ wherein R¹⁰ is a substituted or unsubstituted piperazine, substituted or unsubstituted piperidine, substituted or unsubstituted pyrrolidine or substituted or unsubstituted morpholine. In yet another embodiment is a compound of Formula V-Vh wherein ring B is substituted with at least one CF₃.

In yet another embodiment, ring B is substituted with at least two R⁵. In another embodiment, ring B is substituted with halogen and a substituted or unsubstituted heterocycloalkyl. In another embodiment, ring B is substituted with at least one F, Cl, Br, or I and a substituted or unsubstituted piperazine, substituted or unsubstituted piperidine, substituted or unsubstituted pyrrolidine, or substituted or unsubstituted morpholine.

In a further embodiment is a compound of Formula V-Vh wherein is:

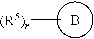

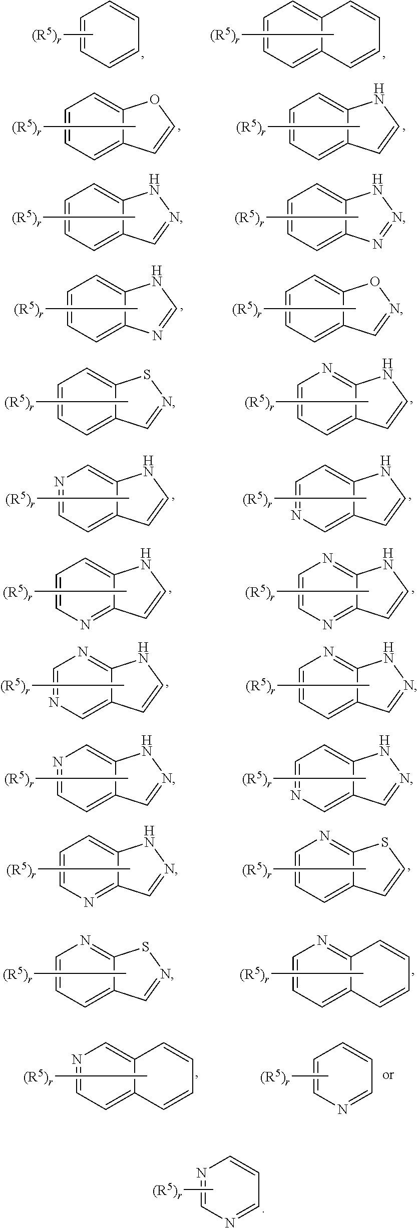

In another embodiment is a compound of Formula V-Vh where R⁵ is halogen, —CN, —OH, a substituted or unsubstituted alkyl, —OR¹⁰, —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or a substituted or unsubstituted heterocycloalkyl.

In one embodiment is a compound of Formula V-Vh wherein at least one R⁵ is —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)₂N(R¹⁰)₂, or a substituted or unsubstituted heterocycloalkyl.

In one embodiment is a compound of Formula V-Vh wherein at least one R⁵ is —N(R¹⁰)₂, or a substituted or unsubstituted heterocycloalkyl. In a further embodiment is a compound of Formula V-Vh wherein at least one of R⁵ is a substituted or unsubstituted piperazine, a substituted or unsubstituted piperidine, a substituted or unsubstituted pyrrolidine, or a substituted or unsubstituted morpholine. In one embodiment is a compound of Formula V-Vh wherein at least one R⁵ is —OR¹⁰. In another embodiment is a compound of Formula V-Vh wherein R⁴ is independently halogen, —CN, —OH, —OCF₃, —OCF₃, —OCF₂H, —CF₃, —SR⁸, a substituted or unsubstituted alkyl, or a substituted or unsubstituted alkoxy.

In another embodiment is a compound having the structure selected from:

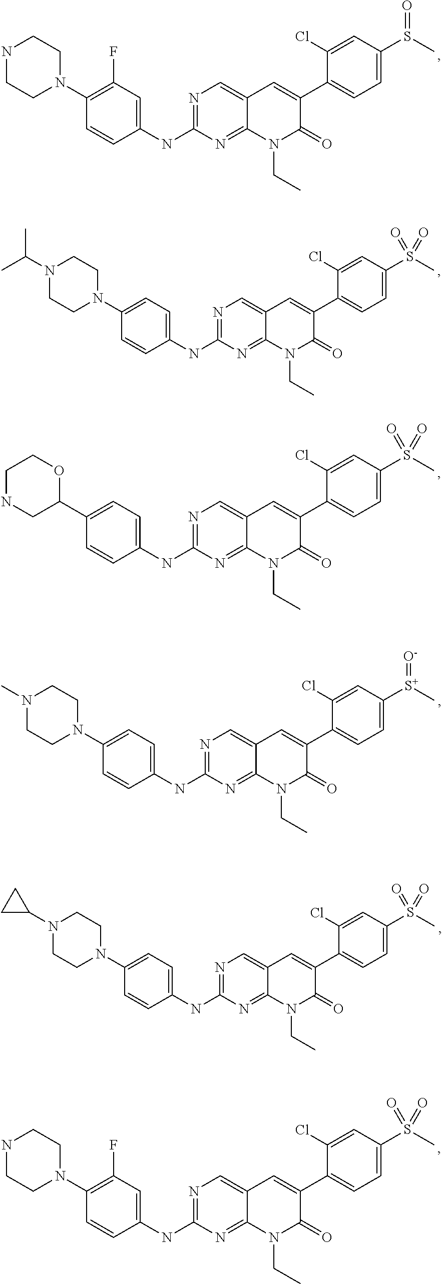

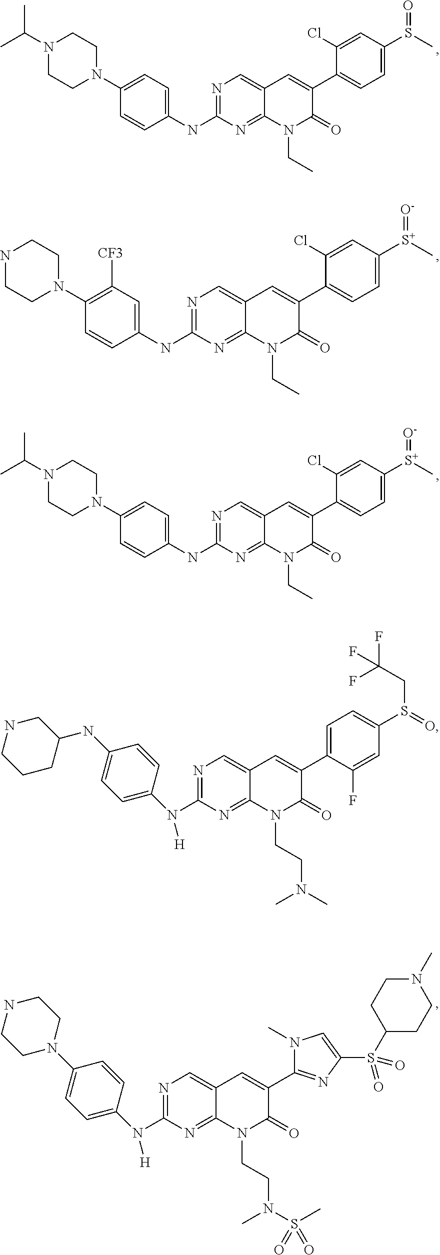

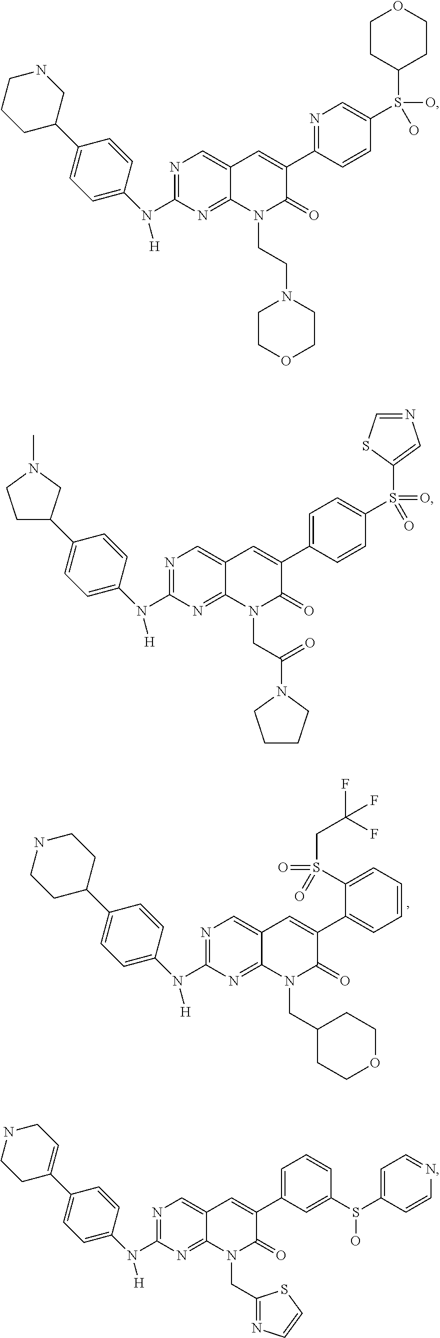

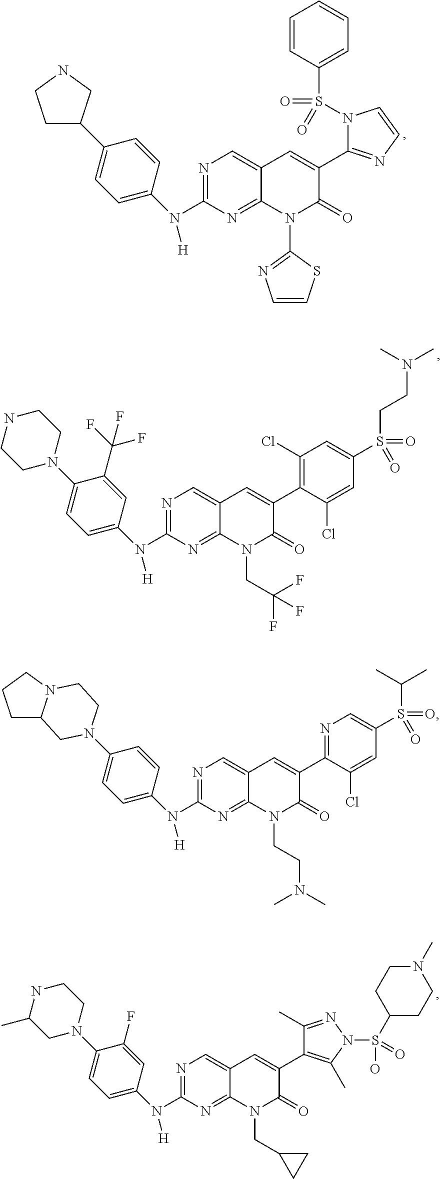

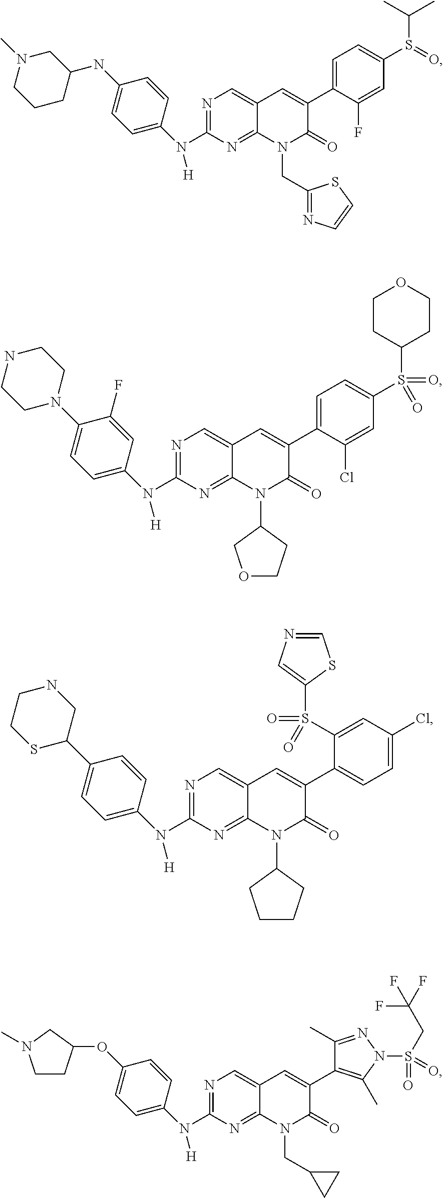

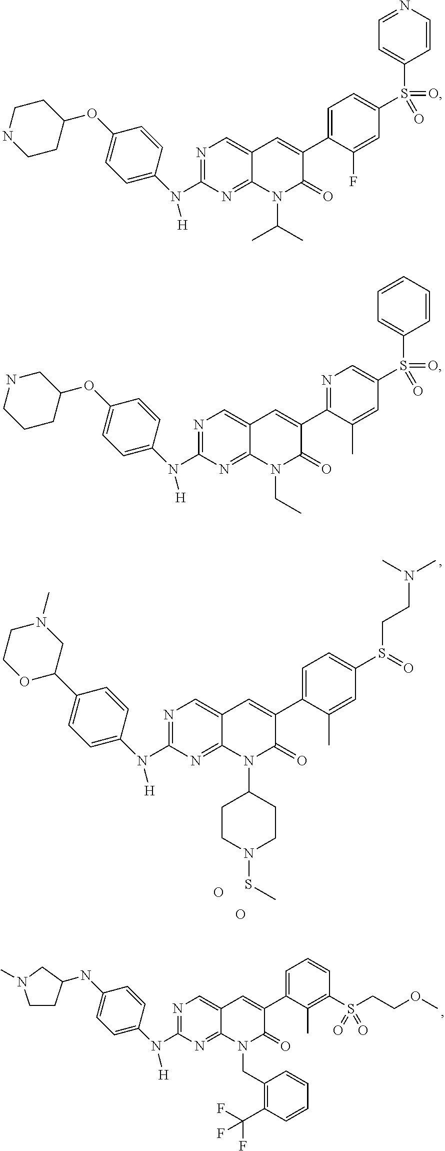

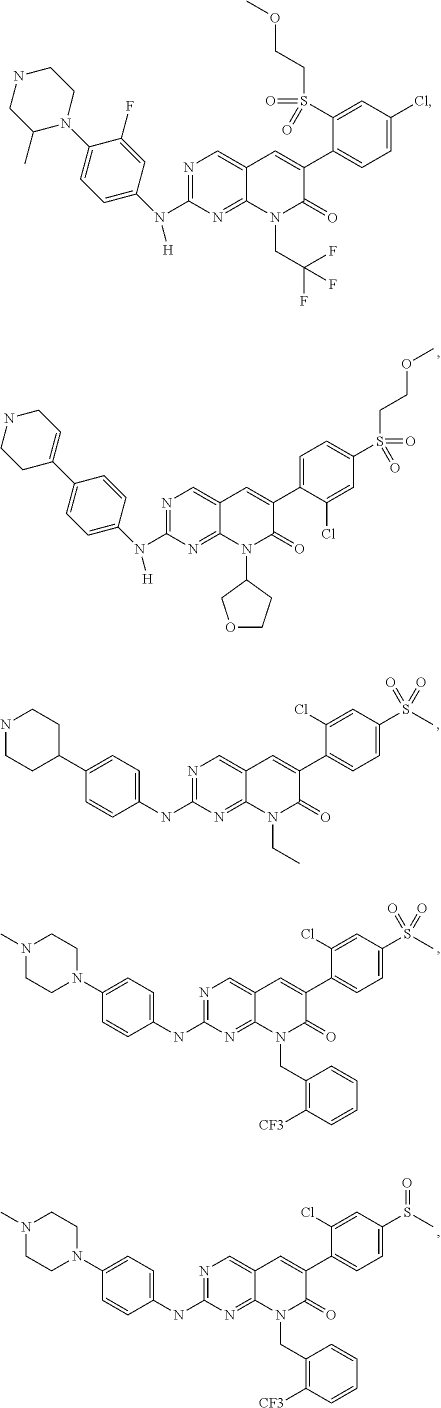

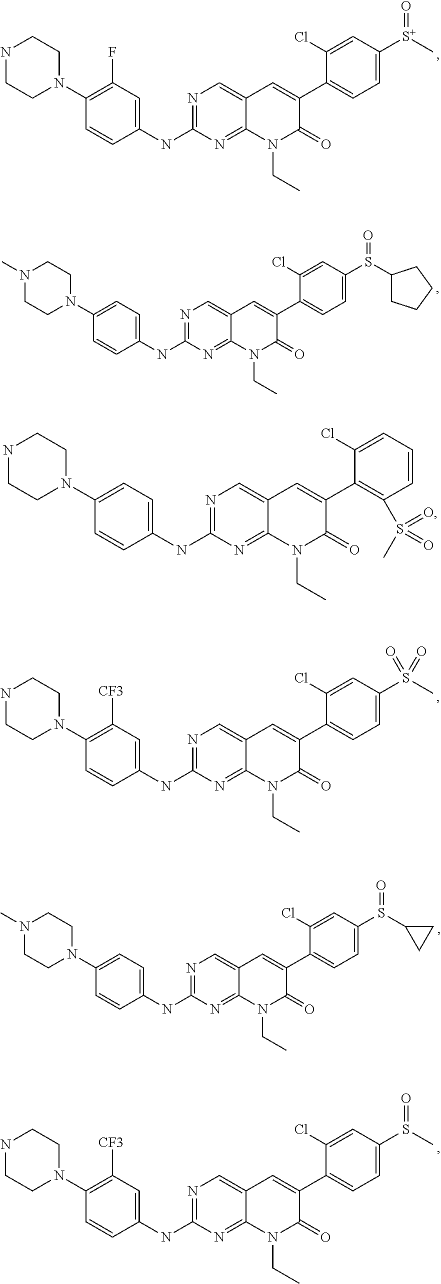

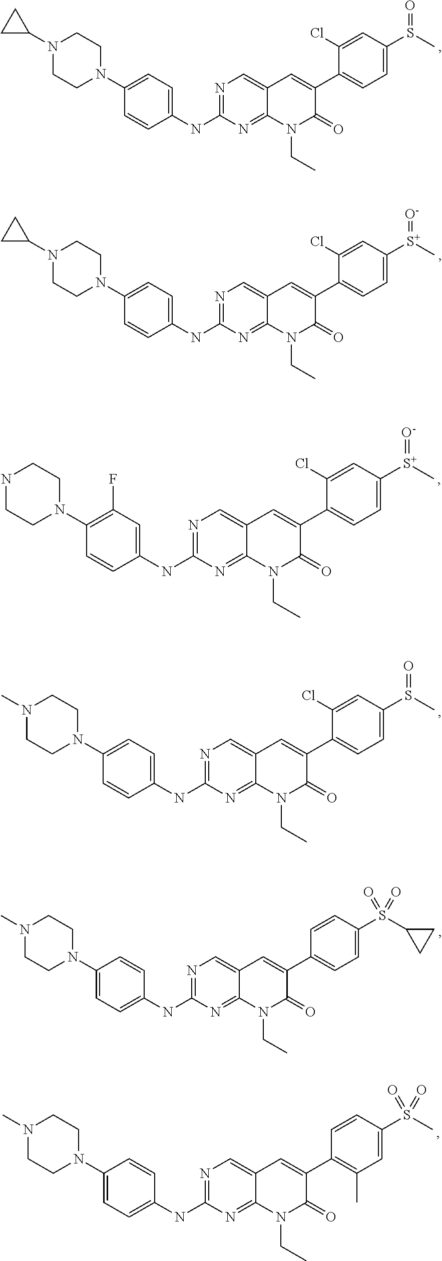

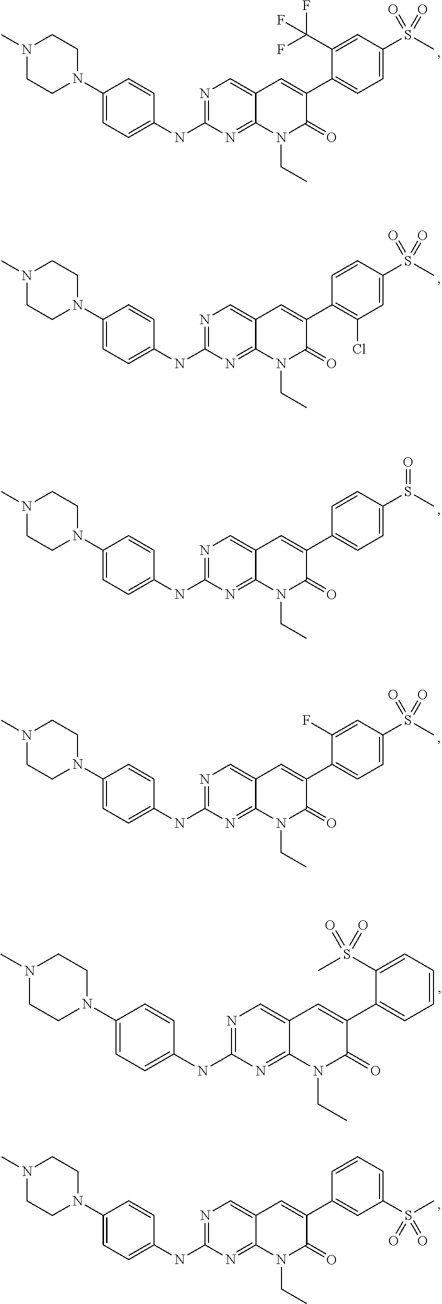

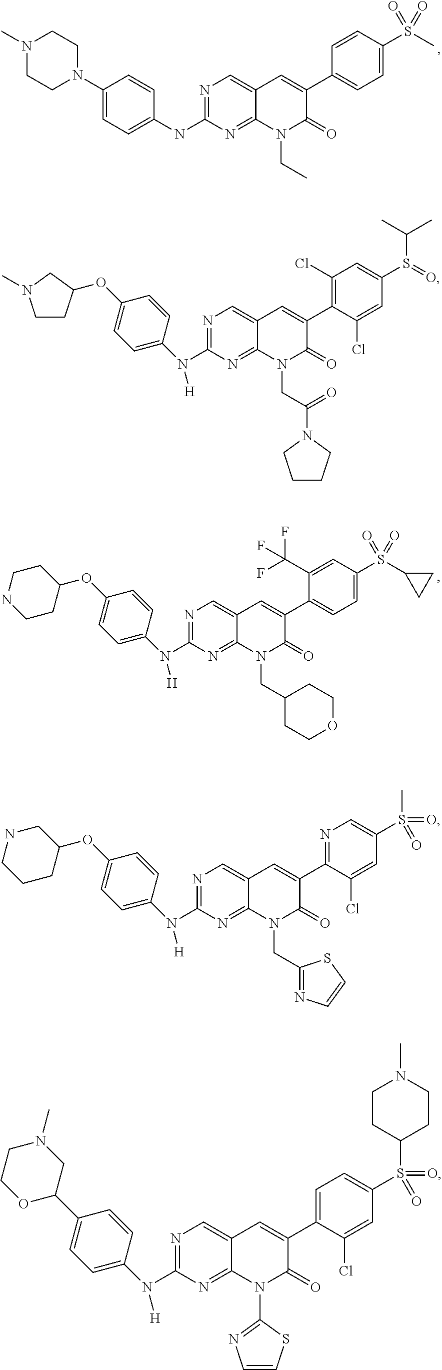

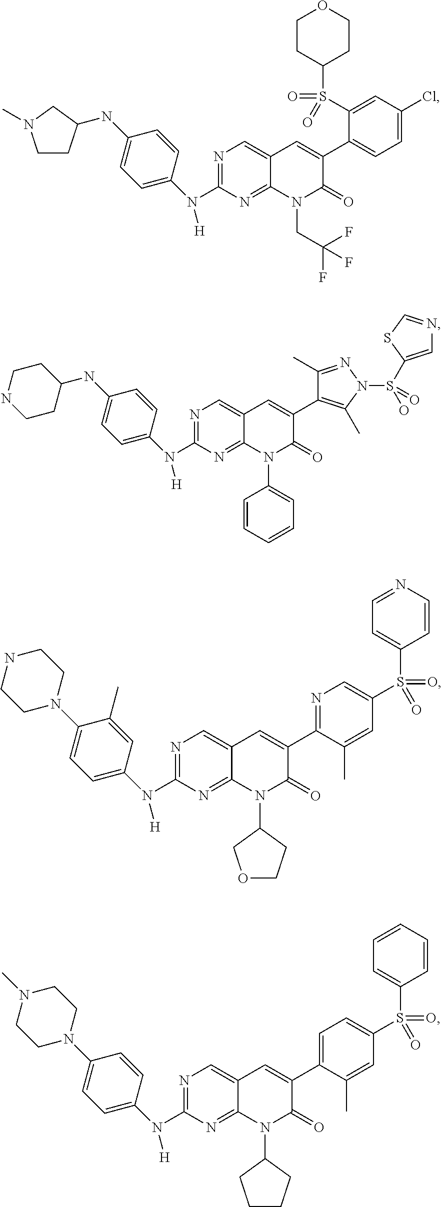

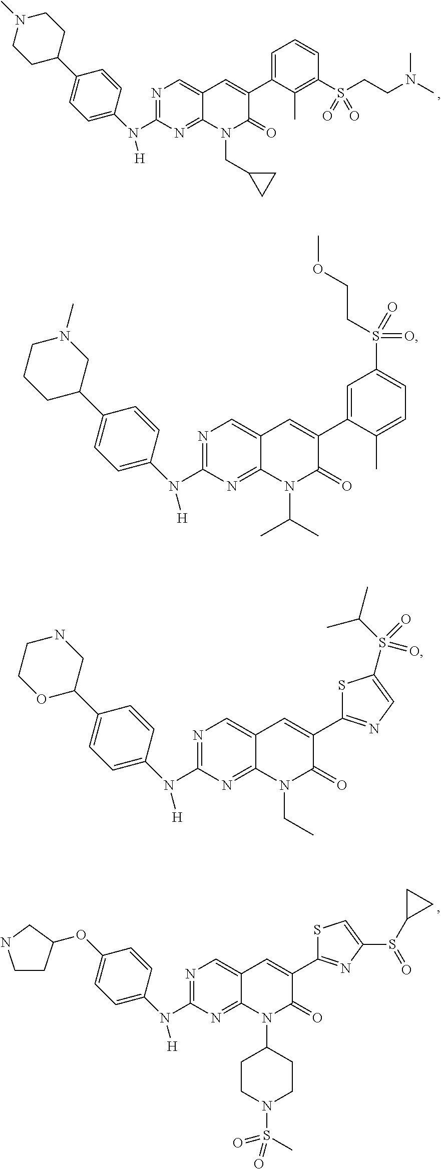

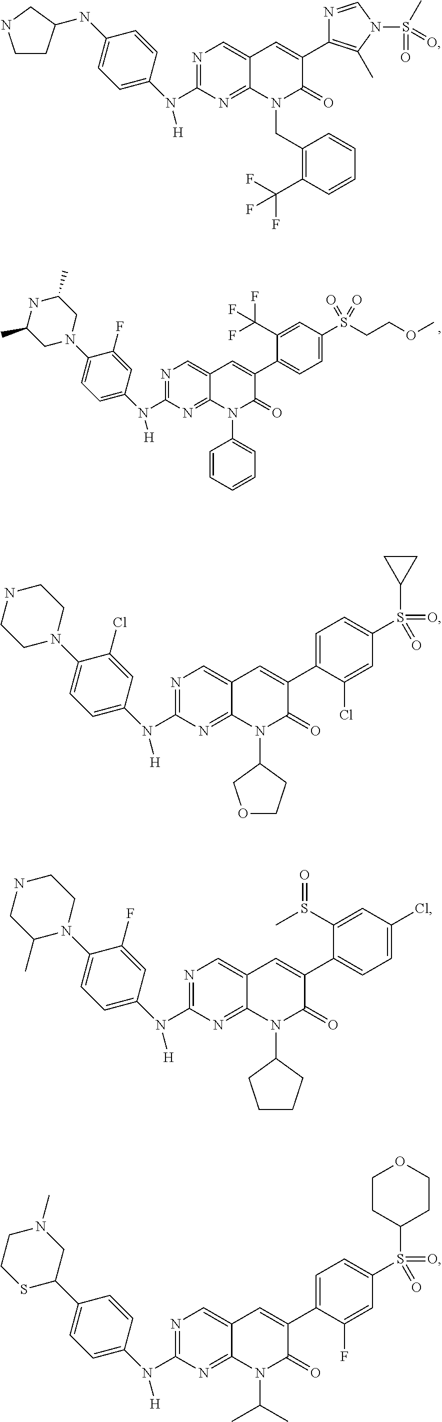

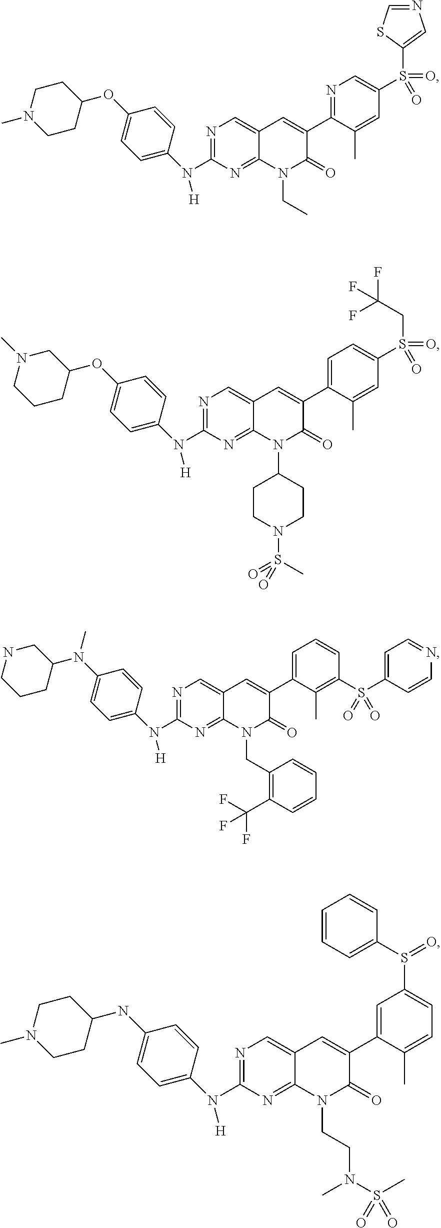 [SMILES: CCn1c(=O)c(-c2ncc(S(=O)(=O)c3cncs3)cc2C)cc2cnc(Nc3ccc(OC4CCN(C)CC4)cc3)nc21 ; Cc1cc(S(=O)(=O)CC(F)(F)F)ccc1-c1cc2cnc(Nc3ccc(OC4CCCN(C)C4)cc3)nc2n(C2CCN(S(C)(=O)=O)CC2)c1=O ; Cc1c(-c2cc3cnc(Nc4ccc(N(C)C5CCCNC5)cc4)nc3n(Cc3ccccc3C(F)(F)F)c2=O)cccc1S(=O)(=O)c1ccncc1 ; Cc1ccc(S(=O)c2ccccc2)cc1-c1cc2cnc(Nc3ccc(NC4CCN(C)CC4)cc3)nc2n(CCN(C)S(C)(=O)=O)c1=O]

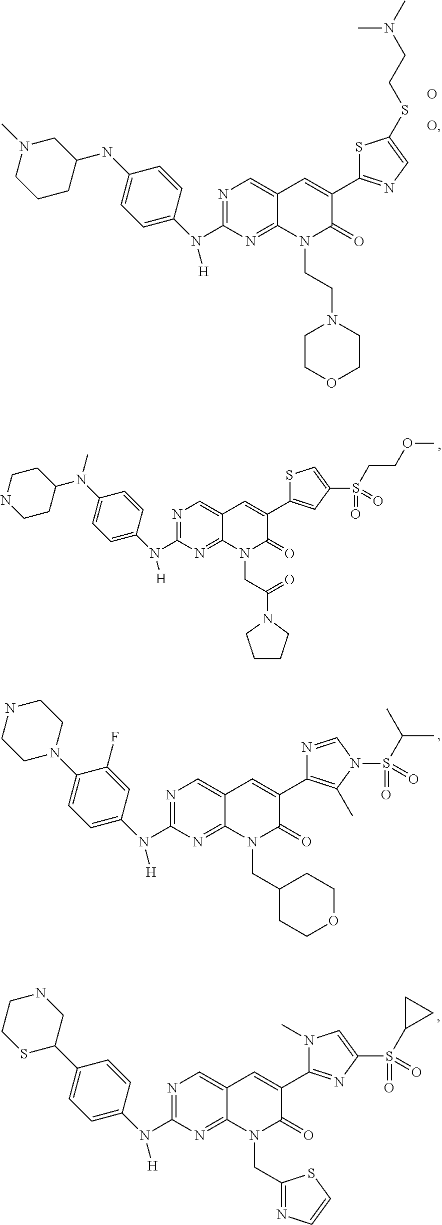

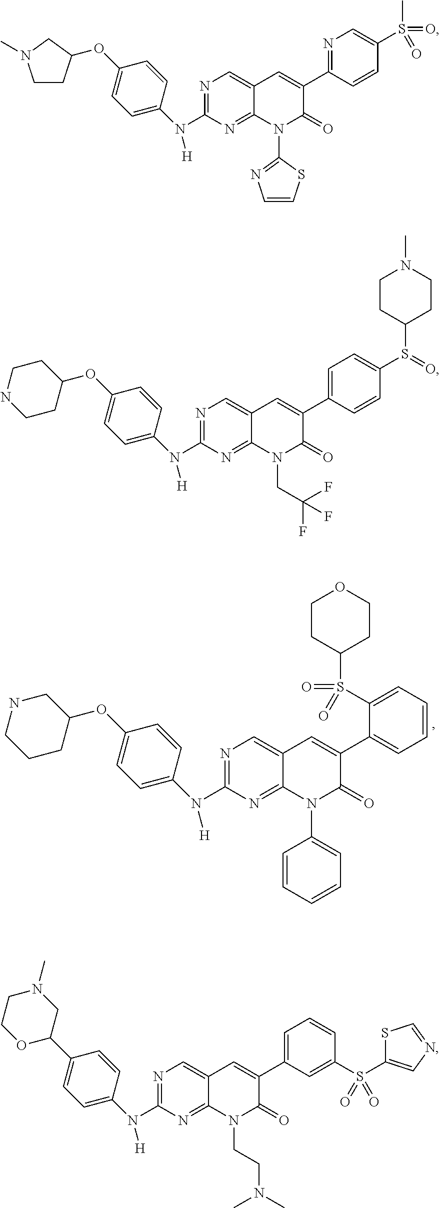 [SMILES: CN1CCC(Oc2ccc(Nc3ncc4cc(-c5ccc(S(C)(=O)=O)cn5)c(=O)n(-c5nccs5)c4n3)cc2)C1 ; CN1CCC(S(=O)c2ccc(-c3cc4cnc(Nc5ccc(OC6CCNCC6)cc5)nc4n(CC(F)(F)F)c3=O)cc2)CC1 ; O=c1c(-c2ccccc2S(=O)(=O)C2CCOCC2)cc2cnc(Nc3ccc(OC4CCCNC4)cc3)nc2n1-c1ccccc1 ; CN(C)CCn1c(=O)c(-c2cccc(S(=O)(=O)c3cncs3)c2)cc2cnc(Nc3ccc(C4CN(C)CCO4)cc3)nc21]

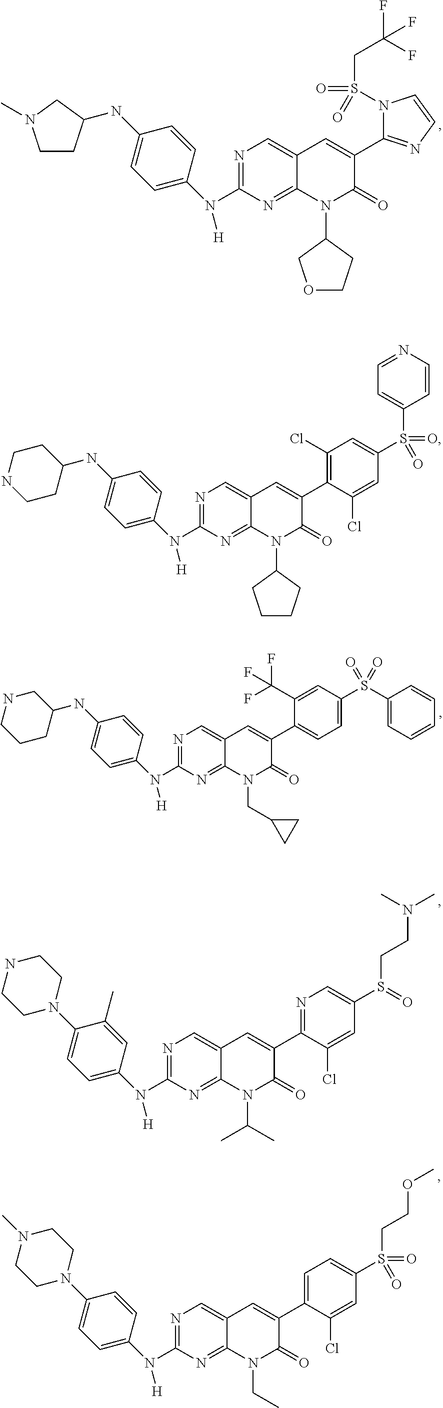

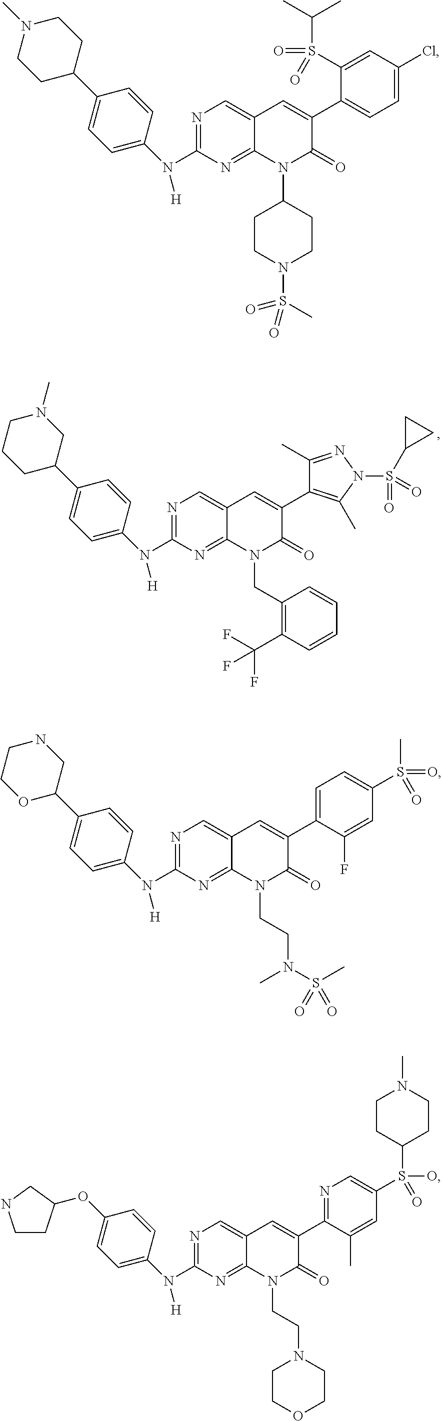

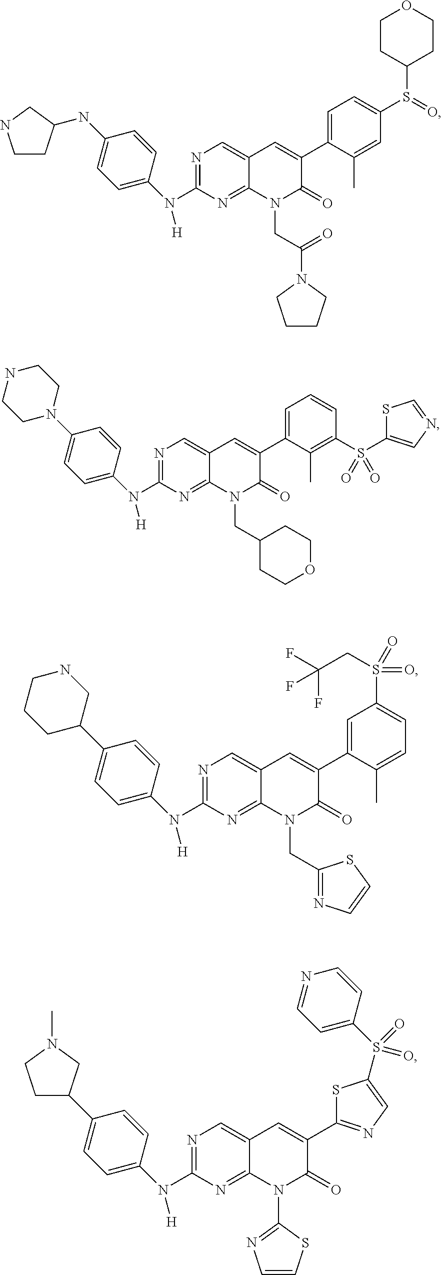 [SMILES: Cc1cc(S(=O)C2CCOCC2)ccc1-c1cc2cnc(Nc3ccc(NC4CCNC4)cc3)nc2n(CC(=O)N2CCCC2)c1=O ; Cc1c(-c2cc3cnc(Nc4ccc(N5CCNCC5)cc4)nc3n(CC3CCOCC3)c2=O)cccc1S(=O)(=O)c1cncs1 ; Cc1ccc(S(=O)(=O)CC(F)(F)F)cc1-c1cc2cnc(Nc3ccc(C4CCCNC4)cc3)nc2n(Cc2nccs2)c1=O ; CN1CCC(c2ccc(Nc3ncc4cc(-c5ncc(S(=O)(=O)c6ccncc6)s5)c(=O)n(-c5nccs5)c4n3)cc2)C1]

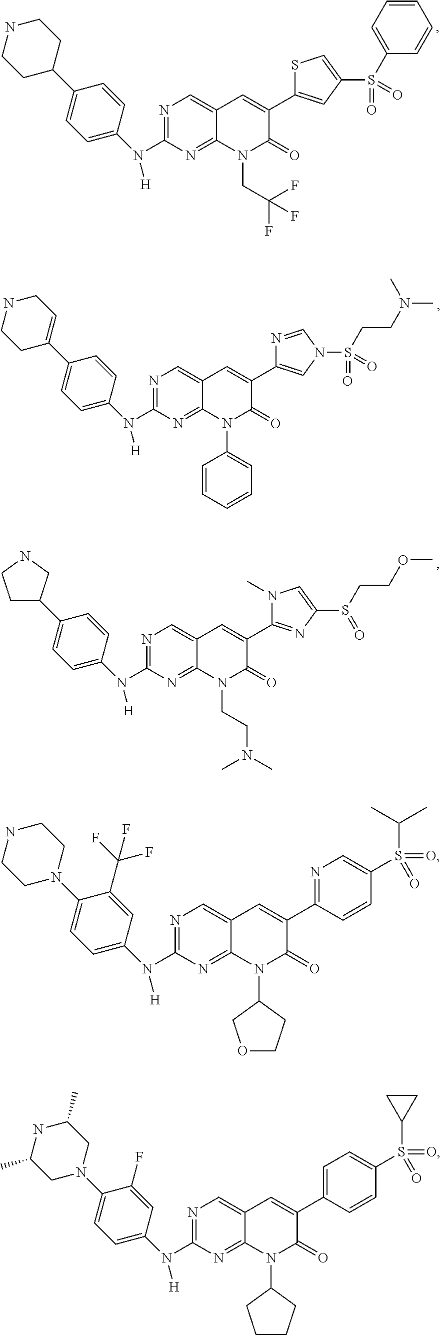

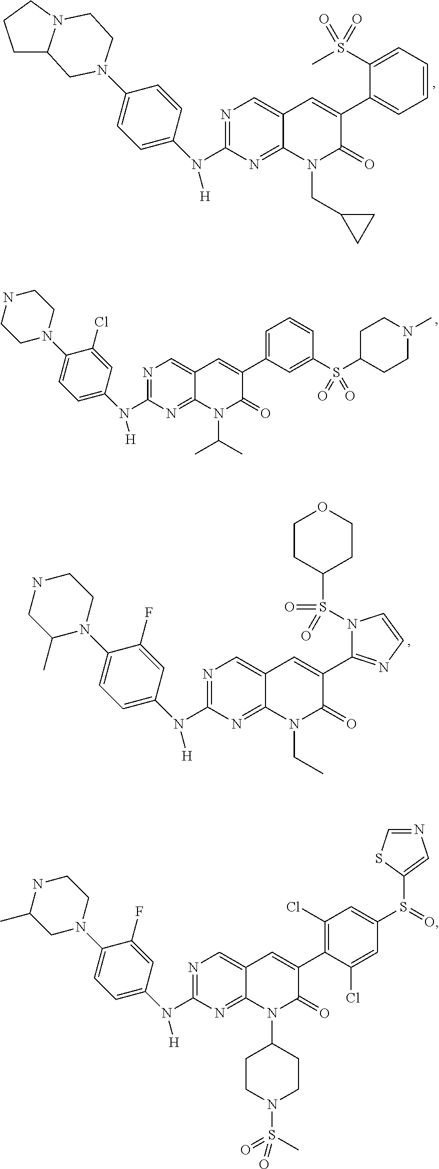

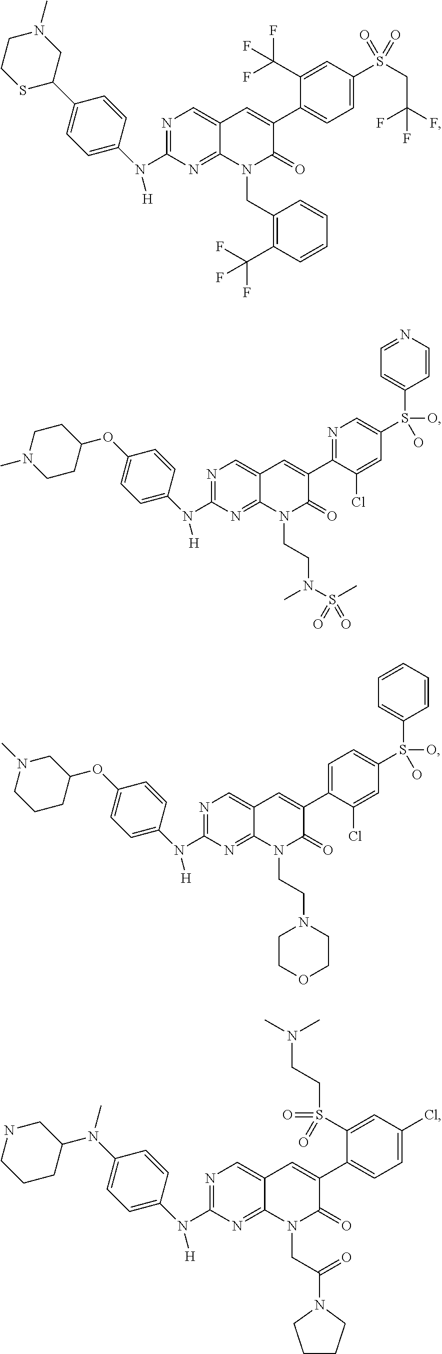

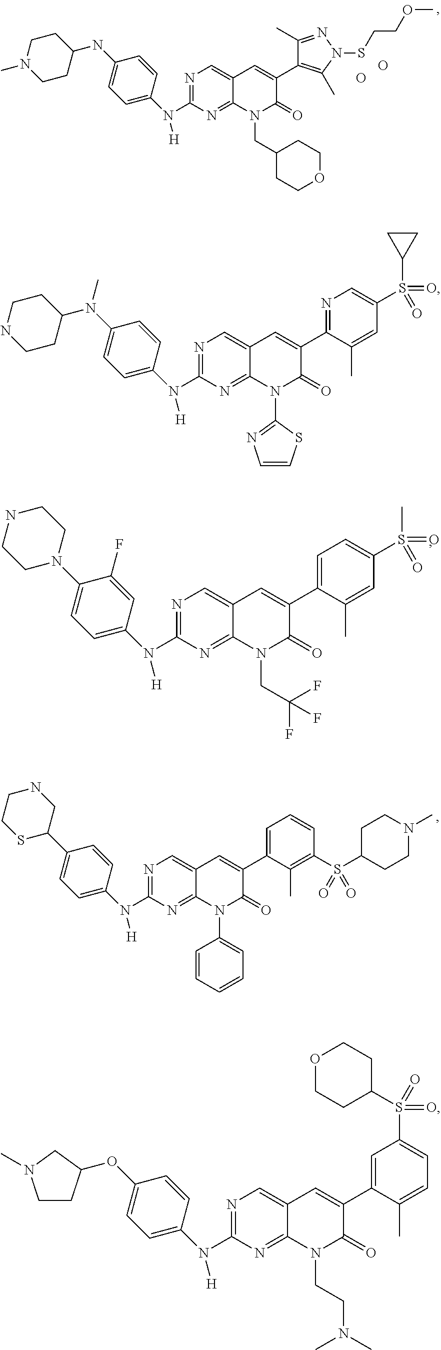

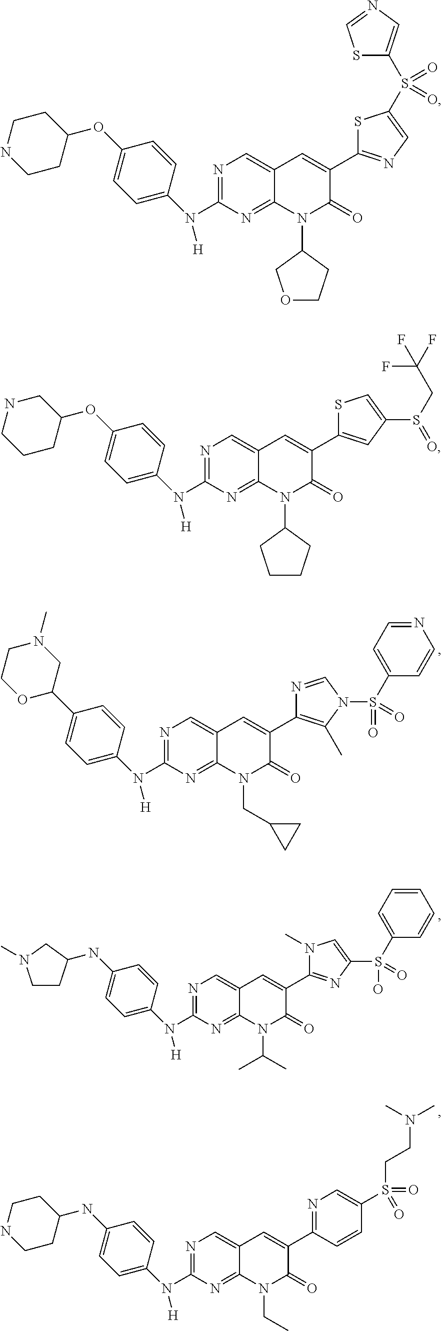

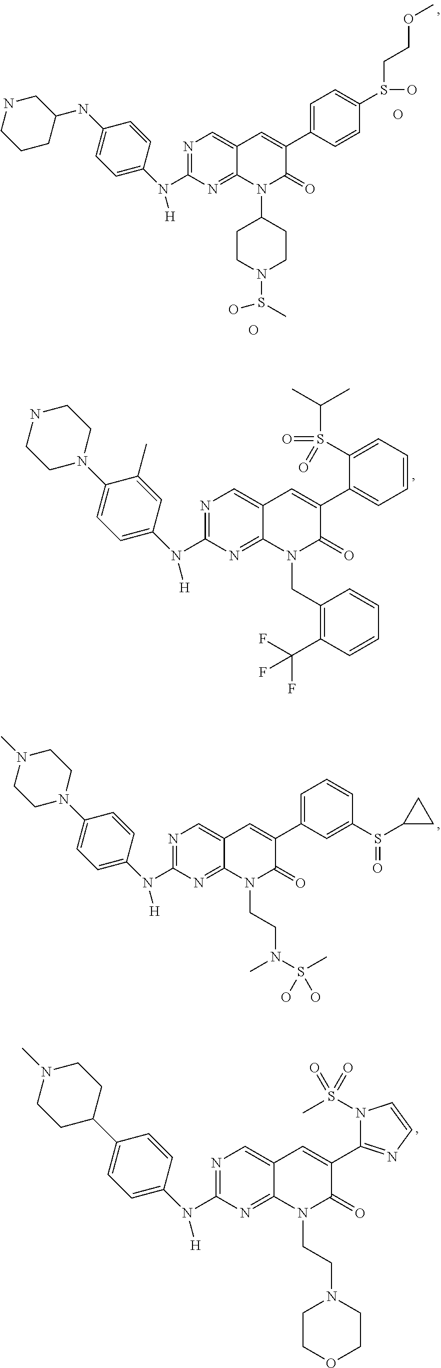

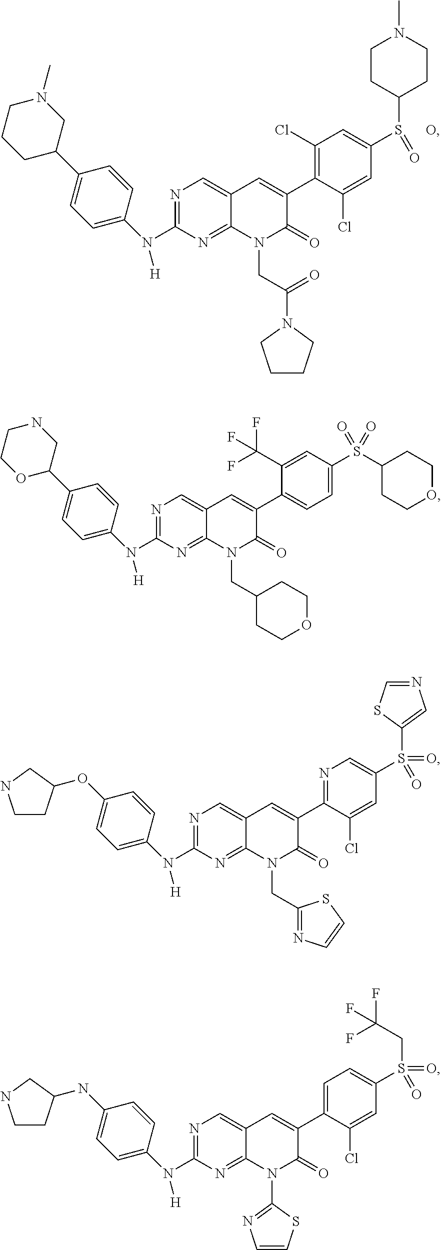

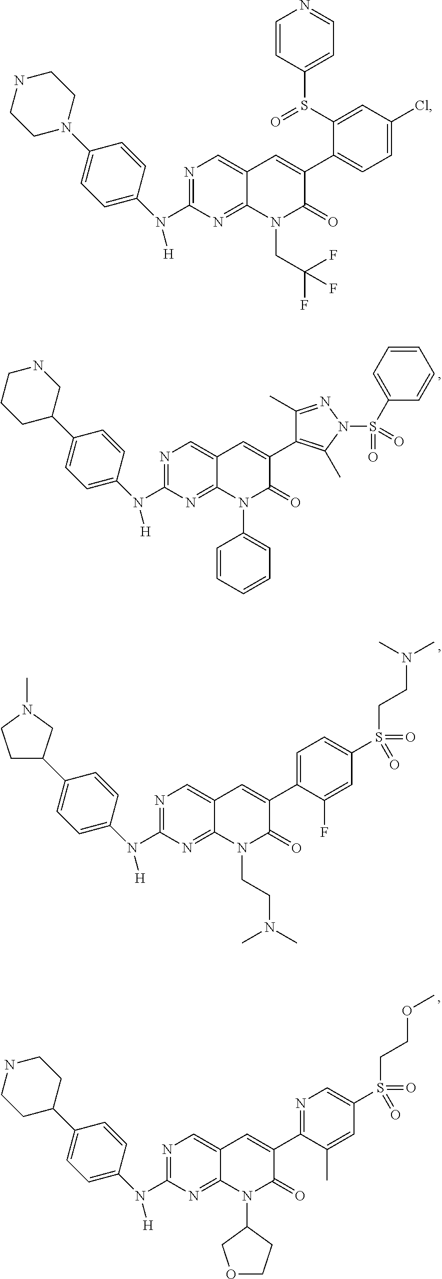 [SMILES: O=c1c(-c2ccc(Cl)cc2S(=O)c2ccncc2)cc2cnc(Nc3ccc(N4CCNCC4)cc3)nc2n1CC(F)(F)F ; Cc1nn(S(=O)(=O)c2ccccc2)c(C)c1-c1cc2cnc(Nc3ccc(C4CCCNC4)cc3)nc2n(-c2ccccc2)c1=O ; CN(C)CCn1c(=O)c(-c2ccc(S(=O)(=O)CCN(C)C)cc2F)cc2cnc(Nc3ccc(C4CCN(C)C4)cc3)nc21 ; COCCS(=O)(=O)c1cnc(-c2cc3cnc(Nc4ccc(C5CCNCC5)cc4)nc3n(C3CCOC3)c2=O)c(C)c1]

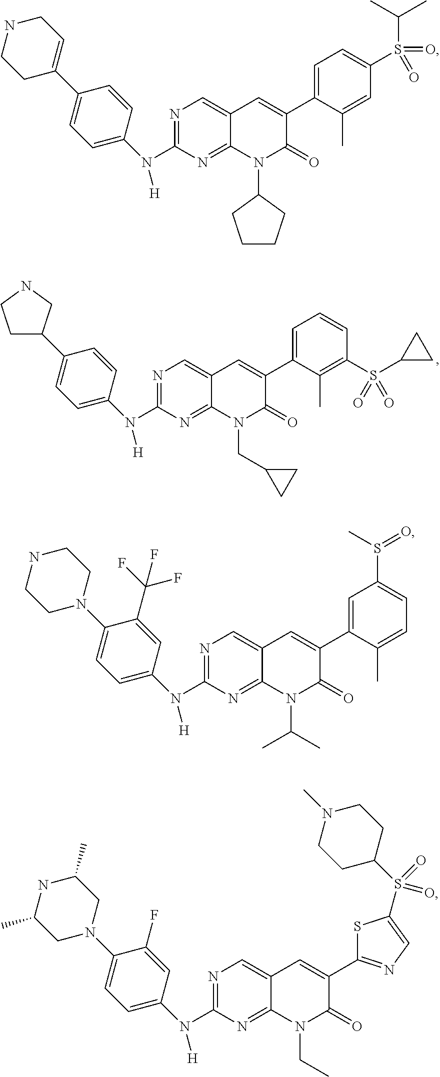

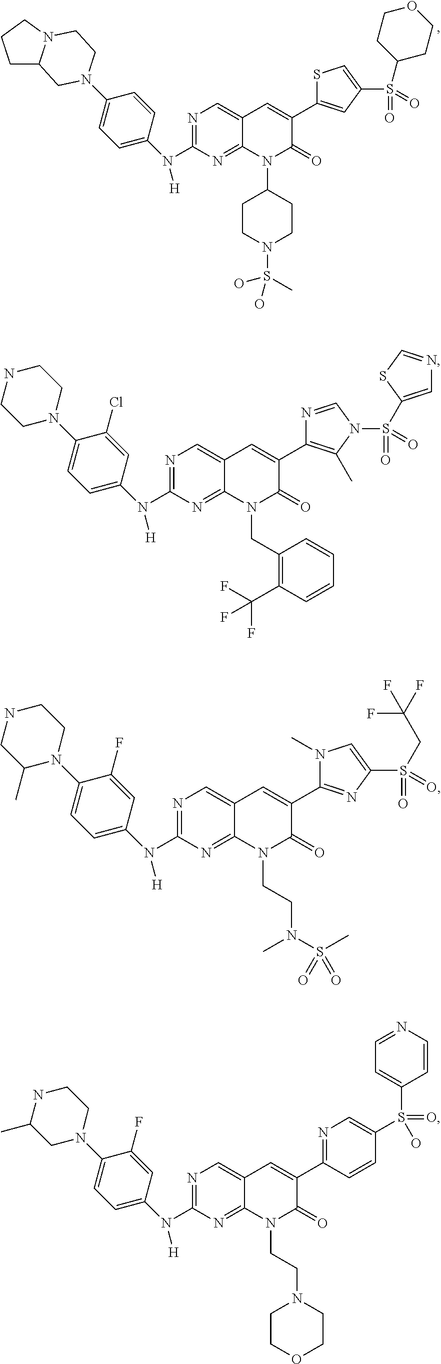

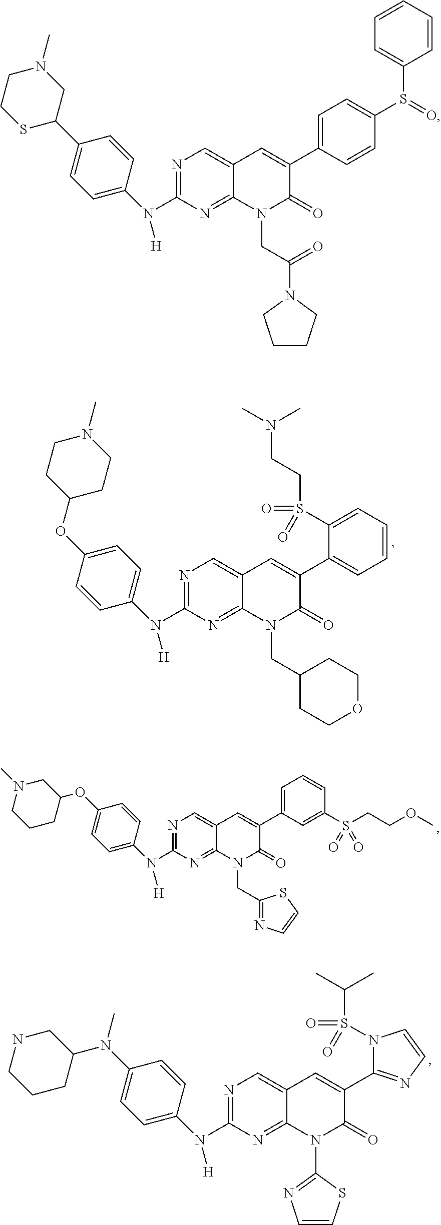

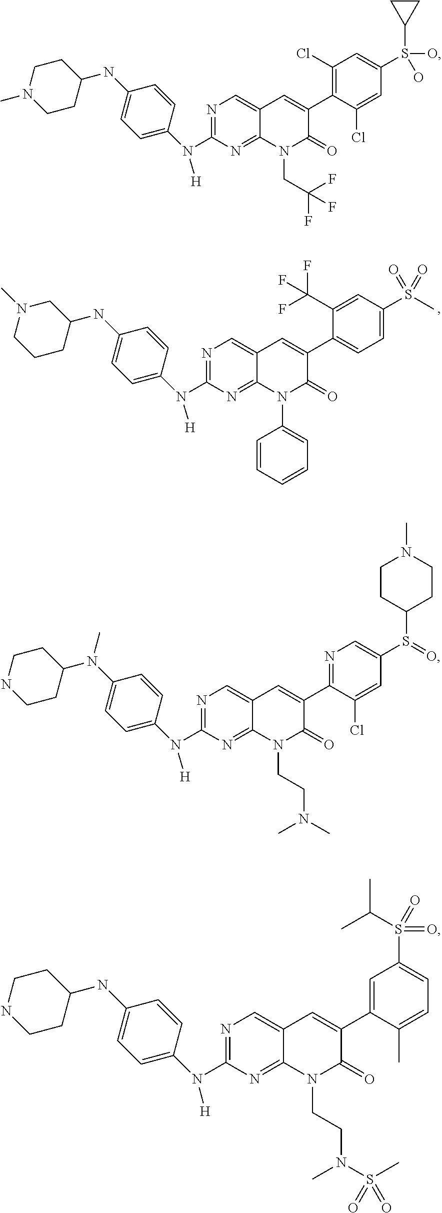

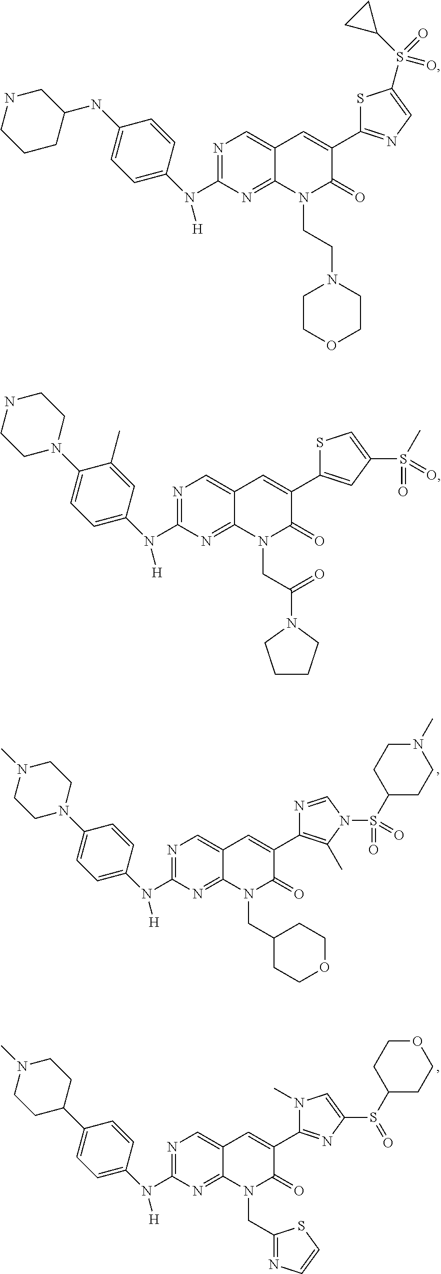

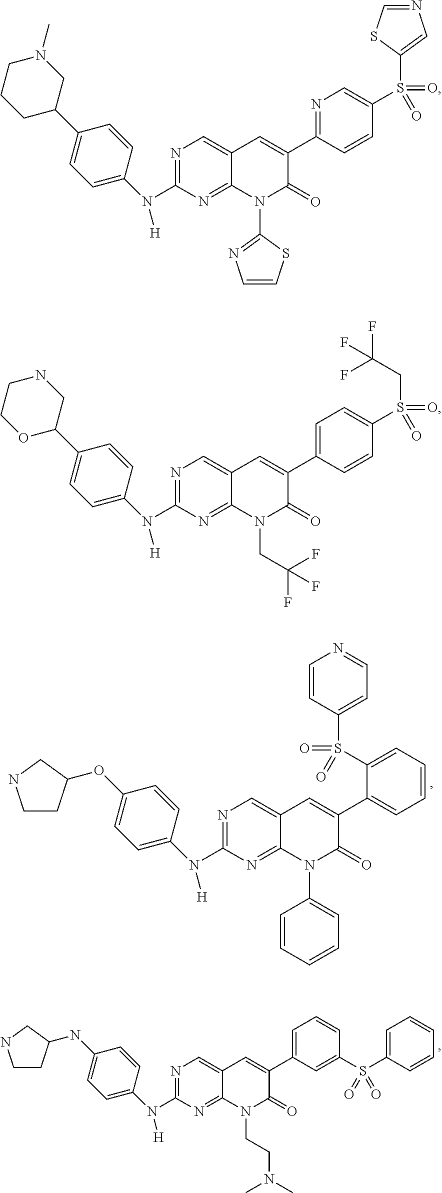 [SMILES: CN1CCCC(c2ccc(Nc3ncc4cc(-c5ccc(S(=O)(=O)c6cncs6)cn5)c(=O)n(-c5nccs5)c4n3)cc2)C1 ; O=c1c(-c2ccc(S(=O)(=O)CC(F)(F)F)cc2)cc2cnc(Nc3ccc(C4CNCCO4)cc3)nc2n1CC(F)(F)F ; O=c1c(-c2ccccc2S(=O)(=O)c2ccncc2)cc2cnc(Nc3ccc(OC4CCNC4)cc3)nc2n1-c1ccccc1 ; CN(C)CCn1c(=O)c(-c2cccc(S(=O)(=O)c3ccccc3)c2)cc2cnc(Nc3ccc(NC4CCNC4)cc3)nc21]

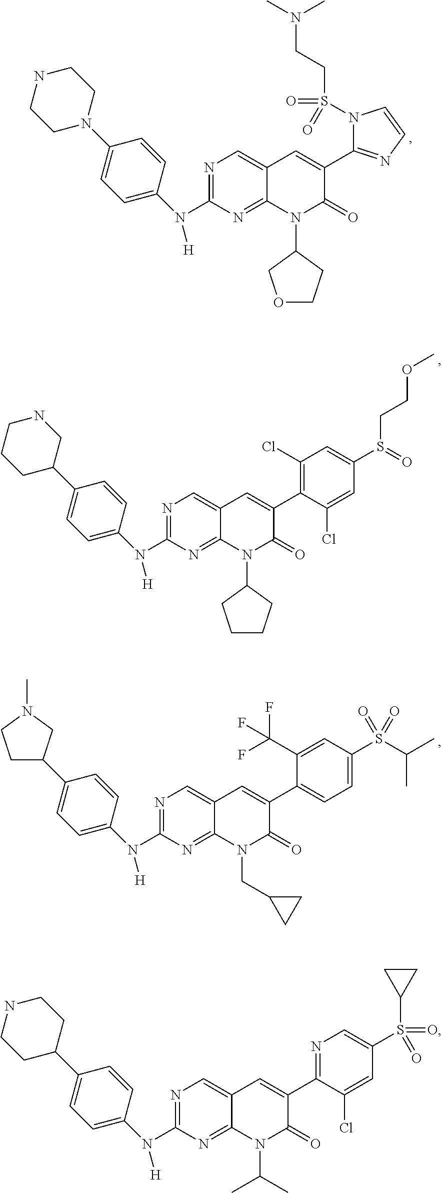 [SMILES: CN(C)CCS(=O)(=O)n1ccnc1-c1cc2cnc(Nc3ccc(N4CCNCC4)cc3)nc2n(C2CCOC2)c1=O ; COCCS(=O)c1cc(Cl)c(-c2cc3cnc(Nc4ccc(C5CCCNC5)cc4)nc3n(C3CCCC3)c2=O)c(Cl)c1 ; CC(C)S(=O)(=O)c1ccc(-c2cc3cnc(Nc4ccc(C5CCN(C)C5)cc4)nc3n(CC3CC3)c2=O)c(C(F)(F)F)c1 ; CC(C)n1c(=O)c(-c2ncc(S(=O)(=O)C3CC3)cc2Cl)cc2cnc(Nc3ccc(C4CCNCC4)cc3)nc21]

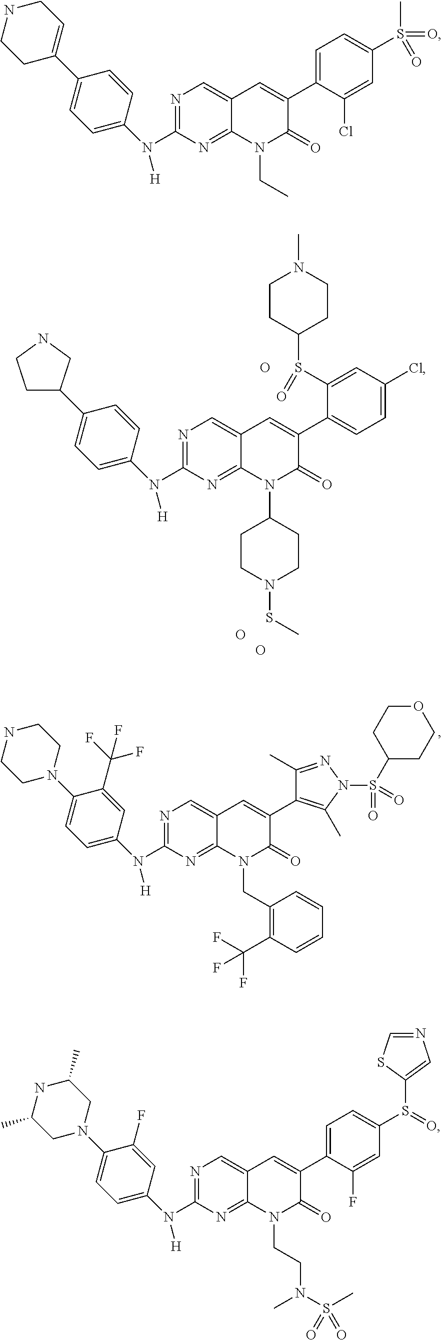

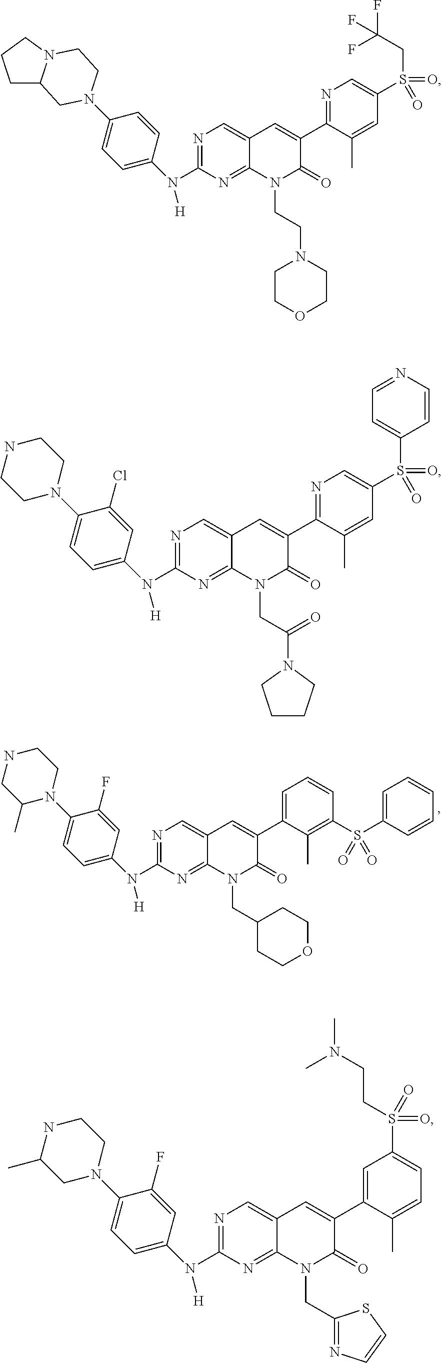 [SMILES: Cc1cc(S(=O)(=O)CC(F)(F)F)cnc1-c1cc2cnc(Nc3ccc(N4CCN5CCCC5C4)cc3)nc2n(CCN2CCOCC2)c1=O ; Cc1cc(S(=O)(=O)c2ccncc2)cnc1-c1cc2cnc(Nc3ccc(N4CCNCC4)c(Cl)c3)nc2n(CC(=O)N2CCCC2)c1=O ; Cc1c(-c2cc3cnc(Nc4ccc(N5CCNCC5C)c(F)c4)nc3n(CC3CCOCC3)c2=O)cccc1S(=O)(=O)c1ccccc1 ; Cc1ccc(S(=O)(=O)CCN(C)C)cc1-c1cc2cnc(Nc3ccc(N4CCNC(C)C4)c(F)c3)nc2n(Cc2nccs2)c1=O]

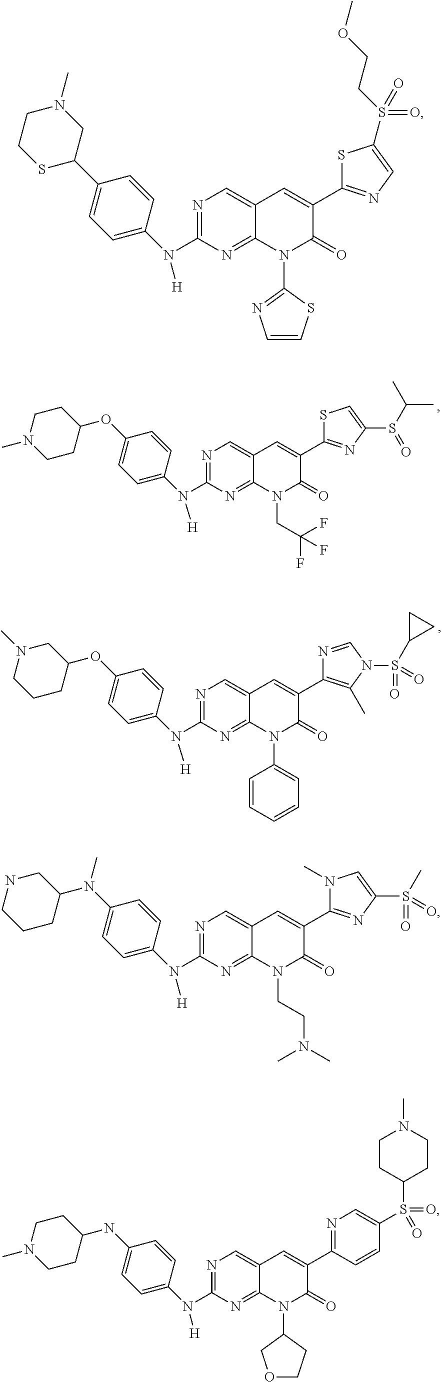

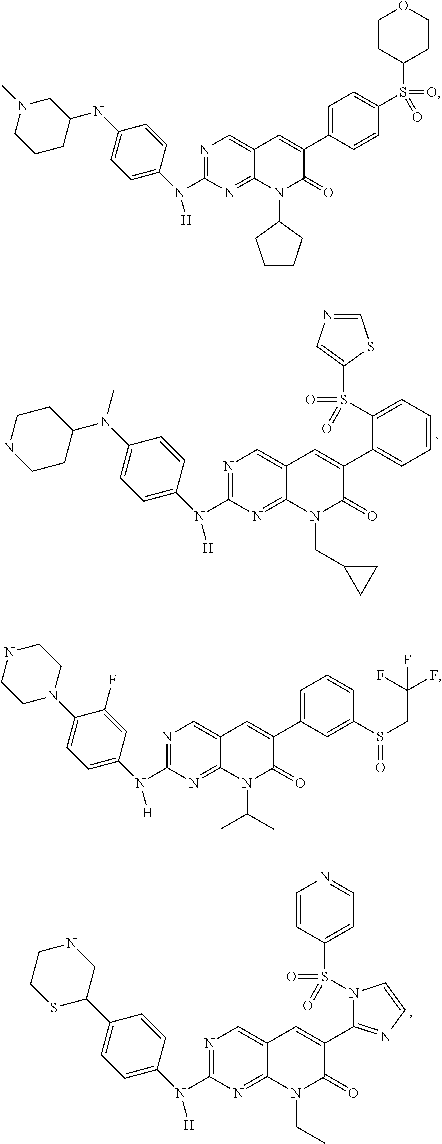 [SMILES: CN1CCCC(Nc2ccc(Nc3ncc4cc(-c5ccc(S(=O)(=O)C6CCOCC6)cc5)c(=O)n(C5CCCC5)c4n3)cc2)C1 ; CN(c1ccc(Nc2ncc3cc(-c4ccccc4S(=O)(=O)c4cncs4)c(=O)n(CC4CC4)c3n2)cc1)C1CCNCC1 ; CC(C)n1c(=O)c(-c2cccc(S(=O)CC(F)(F)F)c2)cc2cnc(Nc3ccc(N4CCNCC4)c(F)c3)nc21 ; CCn1c(=O)c(-c2nccn2S(=O)(=O)c2ccncc2)cc2cnc(Nc3ccc(C4CNCCS4)cc3)nc21]

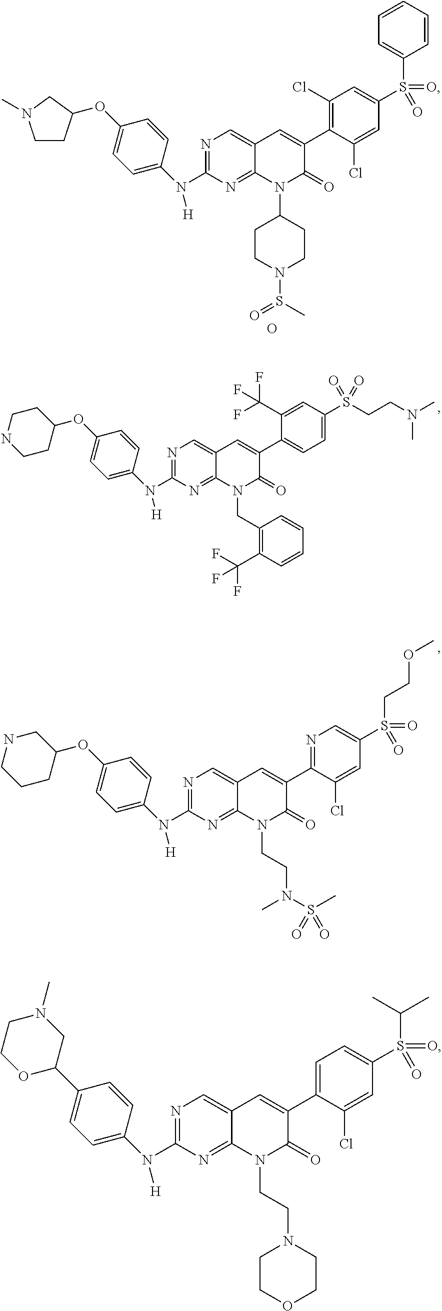

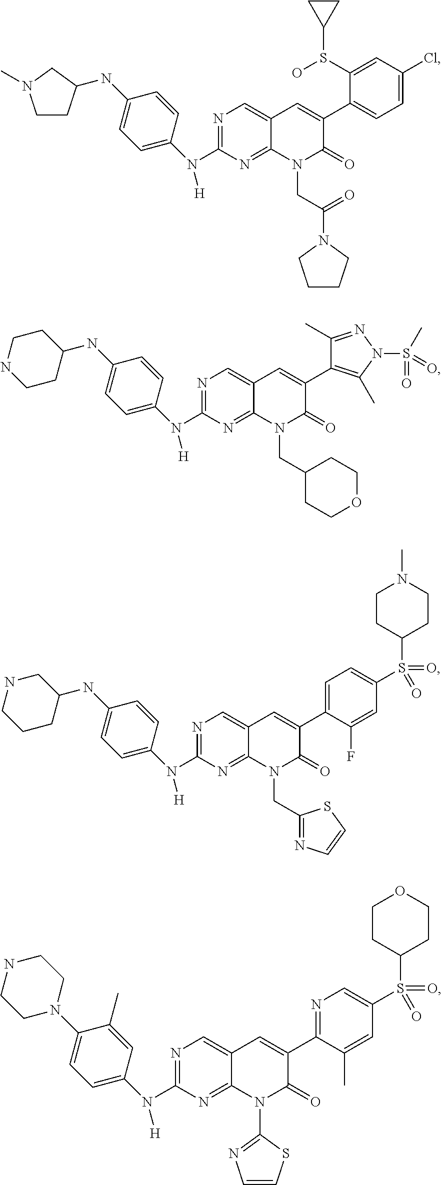

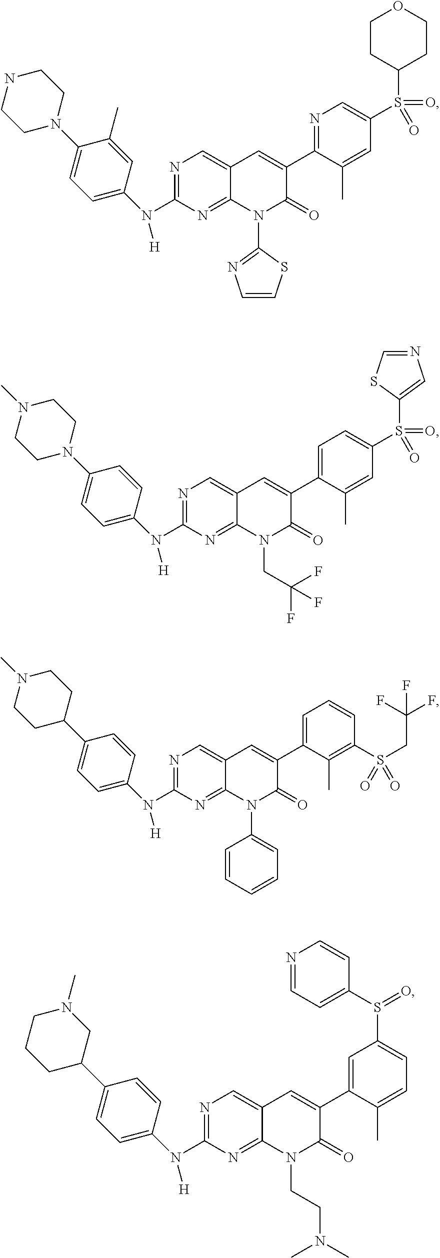 [SMILES: Cc1cc(Nc2ncc3cc(-c4ncc(S(=O)(=O)C5CCOCC5)cc4C)c(=O)n(-c4nccs4)c3n2)ccc1N1CCNCC1 ; Cc1cc(S(=O)(=O)c2cncs2)ccc1-c1cc2cnc(Nc3ccc(N4CCN(C)CC4)cc3)nc2n(CC(F)(F)F)c1=O ; Cc1c(-c2cc3cnc(Nc4ccc(C5CCN(C)CC5)cc4)nc3n(-c3ccccc3)c2=O)cccc1S(=O)(=O)CC(F)(F)F ; Cc1ccc(S(=O)c2ccncc2)cc1-c1cc2cnc(Nc3ccc(C4CCCN(C)C4)cc3)nc2n(CCN(C)C)c1=O]

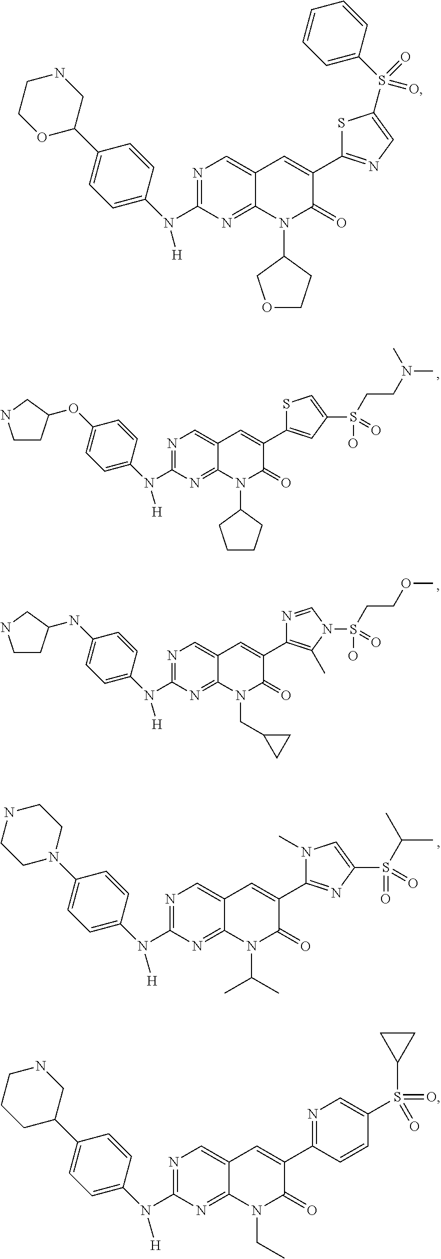

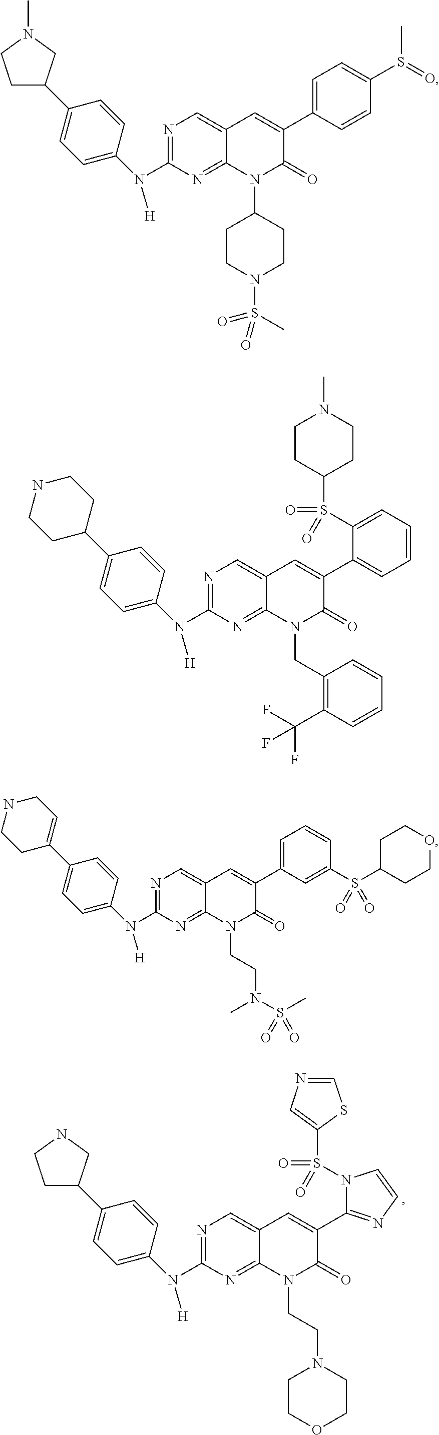

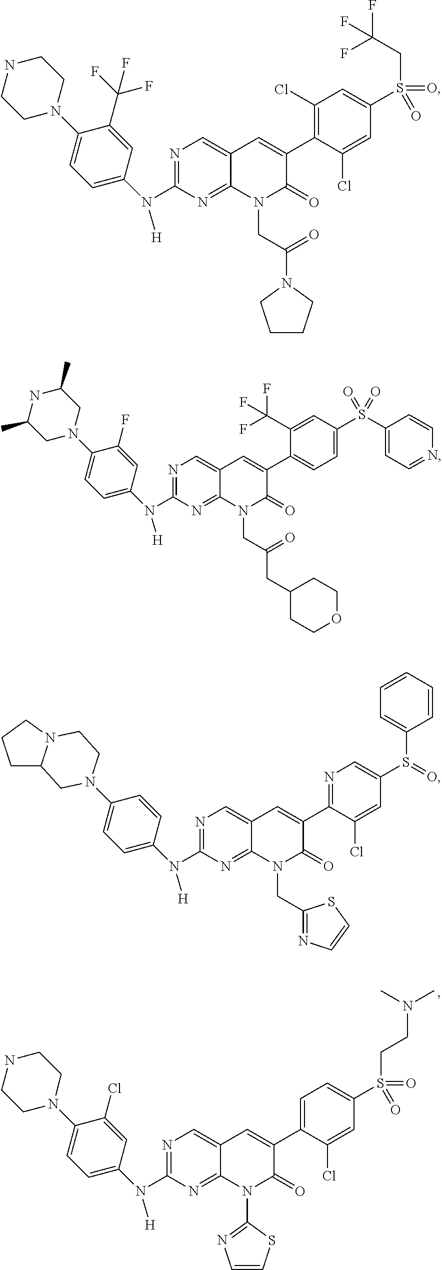 [SMILES: O=C(Cn1c(=O)c(-c2c(Cl)cc(S(=O)(=O)CC(F)(F)F)cc2Cl)cc2cnc(Nc3ccc(N4CCNCC4)c(C(F)(F)F)c3)nc21)N1CCCC1 ; C[C@@H]1CN(c2ccc(Nc3ncc4cc(-c5ccc(S(=O)(=O)c6ccncc6)cc5C(F)(F)F)c(=O)n(CC(=O)CC5CCOCC5)c4n3)cc2F)C[C@H](C)N1 ; O=c1c(-c2ncc(S(=O)c3ccccc3)cc2Cl)cc2cnc(Nc3ccc(N4CCN5CCCC5C4)cc3)nc2n1Cc1nccs1 ; CN(C)CCS(=O)(=O)c1ccc(-c2cc3cnc(Nc4ccc(N5CCNCC5)c(Cl)c4)nc3n(-c3nccs3)c2=O)c(Cl)c1]

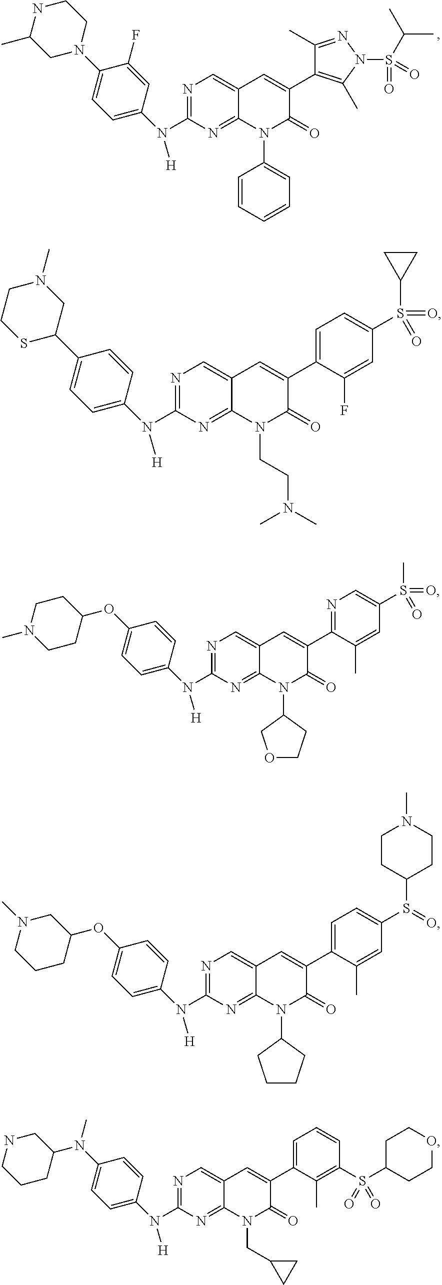

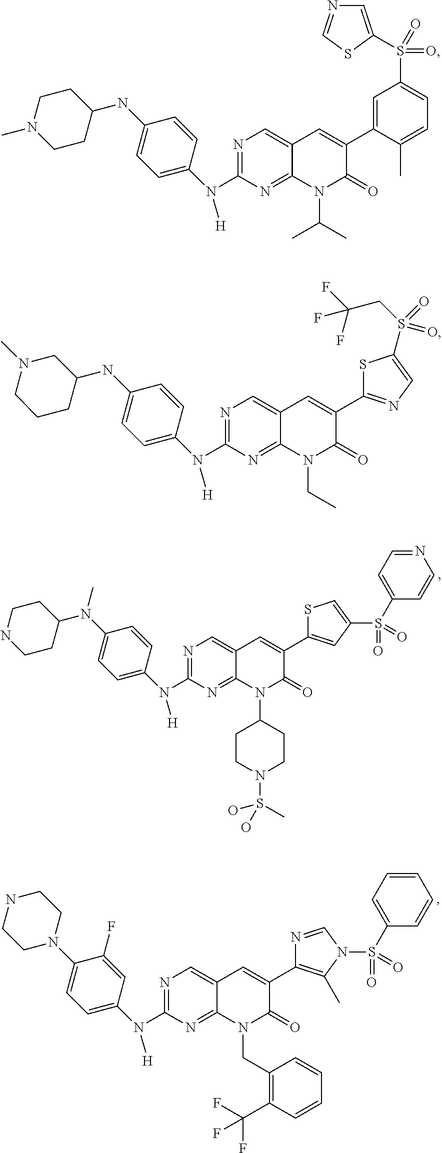 [SMILES: Cc1ccc(S(=O)(=O)c2cncs2)cc1-c1cc2cnc(Nc3ccc(NC4CCN(C)CC4)cc3)nc2n(C(C)C)c1=O ; CCn1c(=O)c(-c2ncc(S(=O)(=O)CC(F)(F)F)s2)cc2cnc(Nc3ccc(NC4CCCN(C)C4)cc3)nc21 ; CN(c1ccc(Nc2ncc3cc(-c4cc(S(=O)(=O)c5ccncc5)cs4)c(=O)n(C4CCN(S(C)(O)O)CC4)c3n2)cc1)C1CCNCC1 ; Cc1c(-c2cc3cnc(Nc4ccc(N5CCNCC5)c(F)c4)nc3n(Cc3ccccc3C(F)(F)F)c2=O)ncn1S(=O)(=O)c1ccccc1]

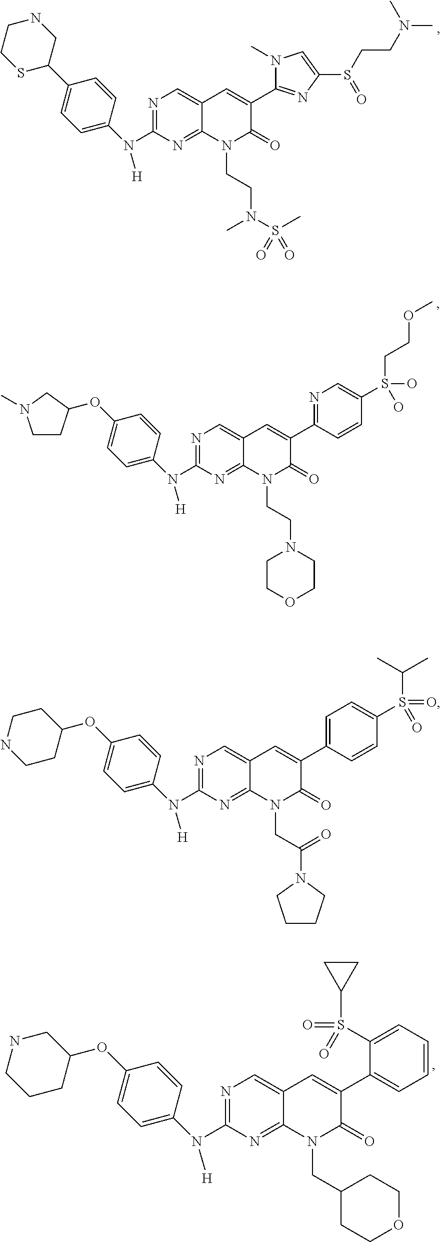

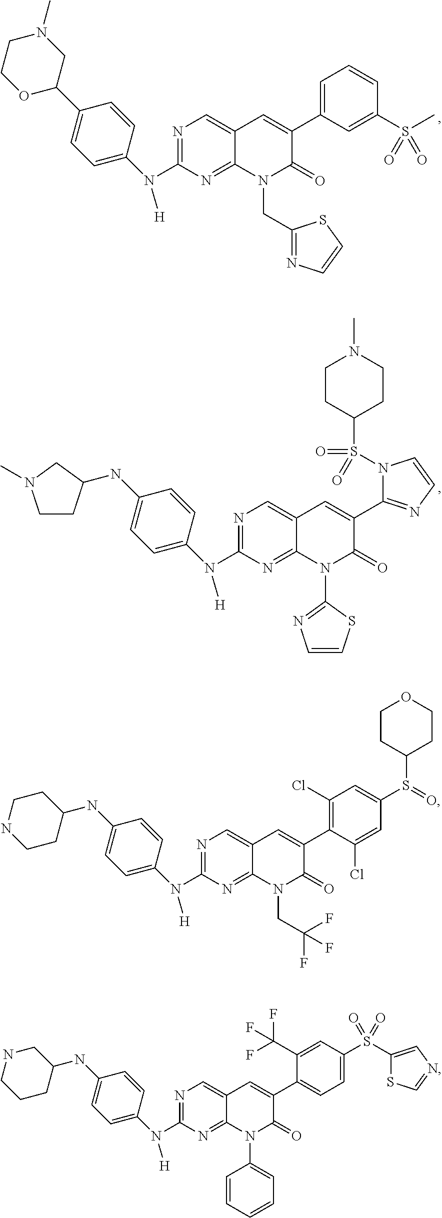

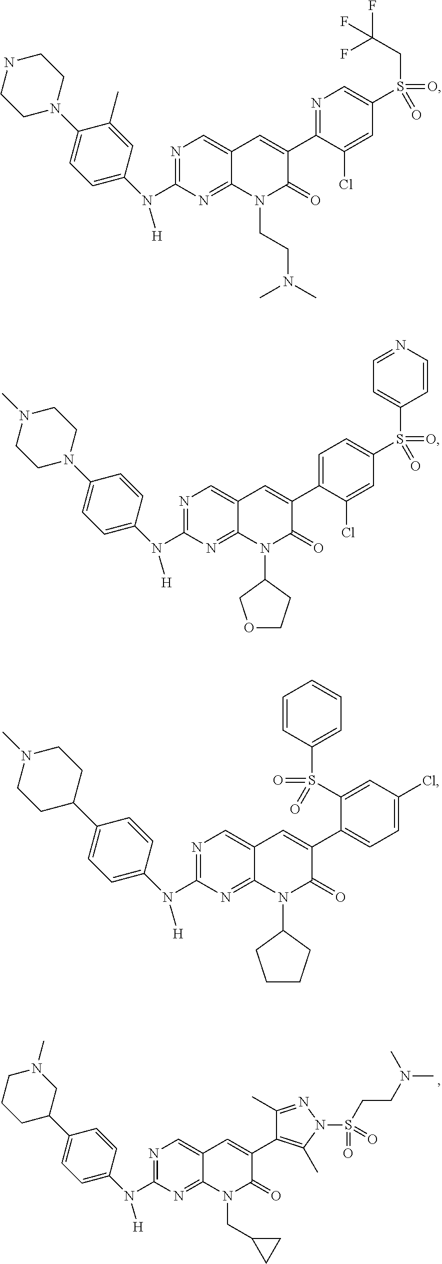 [SMILES: Cc1cc(Nc2ncc3cc(-c4ncc(S(=O)(=O)CC(F)(F)F)cc4Cl)c(=O)n(CCN(C)C)c3n2)ccc1N1CCNCC1 ; CN1CCN(c2ccc(Nc3ncc4cc(-c5ccc(S(=O)(=O)c6ccncc6)cc5Cl)c(=O)n(C5CCOC5)c4n3)cc2)CC1 ; CN1CCC(c2ccc(Nc3ncc4cc(-c5ccc(Cl)cc5S(=O)(=O)c5ccccc5)c(=O)n(C5CCCC5)c4n3)cc2)CC1 ; Cc1nn(S(=O)(=O)CCN(C)C)c(C)c1-c1cc2cnc(Nc3ccc(C4CCCN(C)C4)cc3)nc2n(CC2CC2)c1=O]

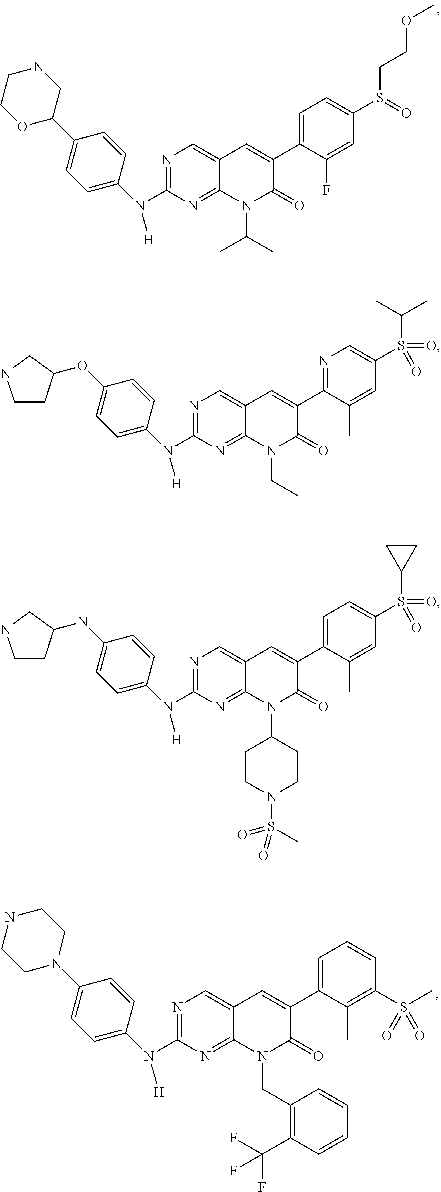 [SMILES: COCCS(=O)c1ccc(-c2cc3cnc(Nc4ccc(C5CNCCO5)cc4)nc3n(C(C)C)c2=O)c(F)c1 ; CCn1c(=O)c(-c2ncc(S(=O)(=O)C(C)C)cc2C)cc2cnc(Nc3ccc(OC4CCNC4)cc3)nc21 ; Cc1cc(S(=O)(=O)C2CC2)ccc1-c1cc2cnc(Nc3ccc(NC4CCNC4)cc3)nc2n(C2CCN(S(C)(=O)=O)CC2)c1=O ; Cc1c(-c2cc3cnc(Nc4ccc(N5CCNCC5)cc4)nc3n(Cc3ccccc3C(F)(F)F)c2=O)cccc1S(C)(=O)=O]

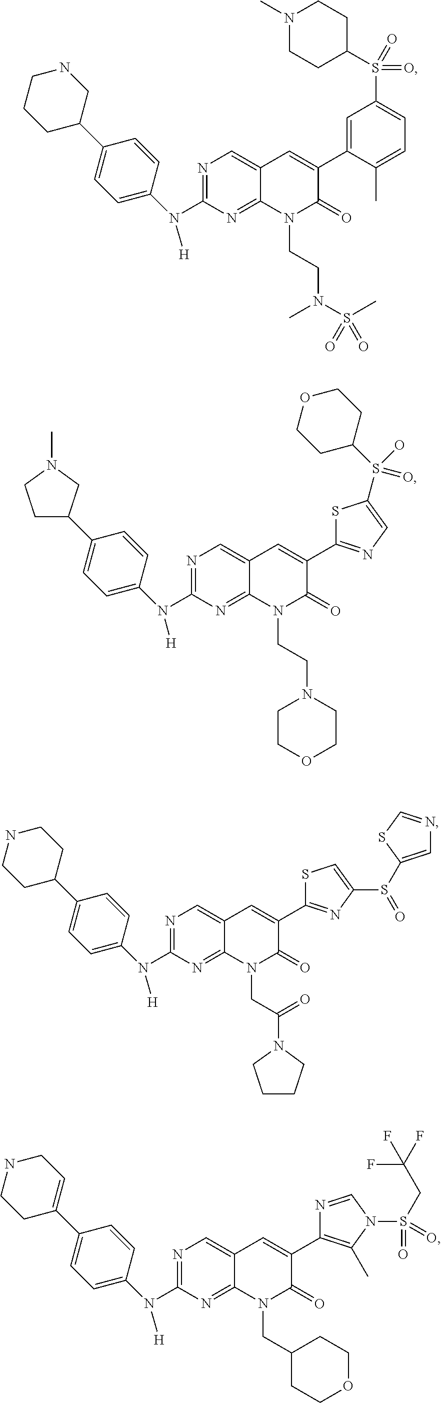

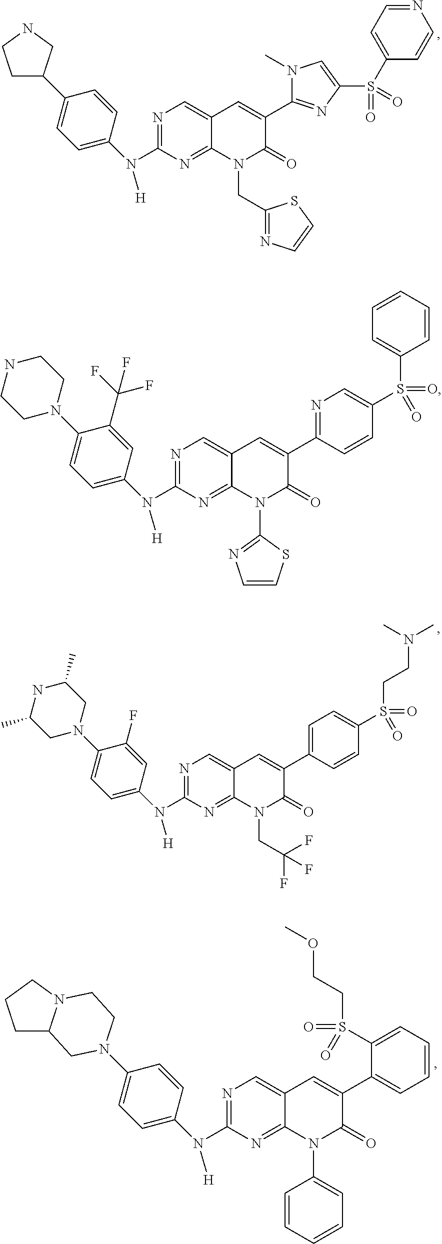

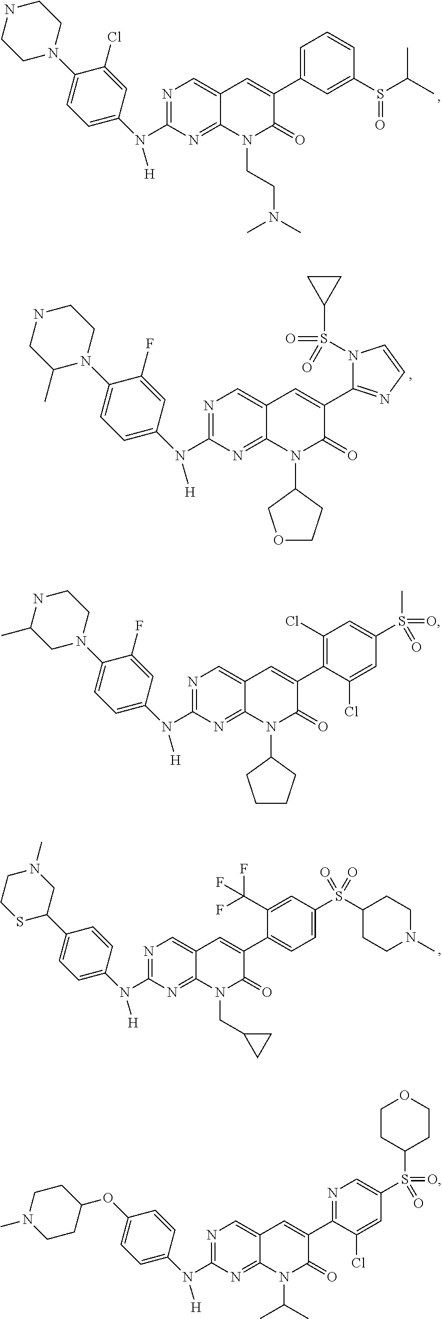

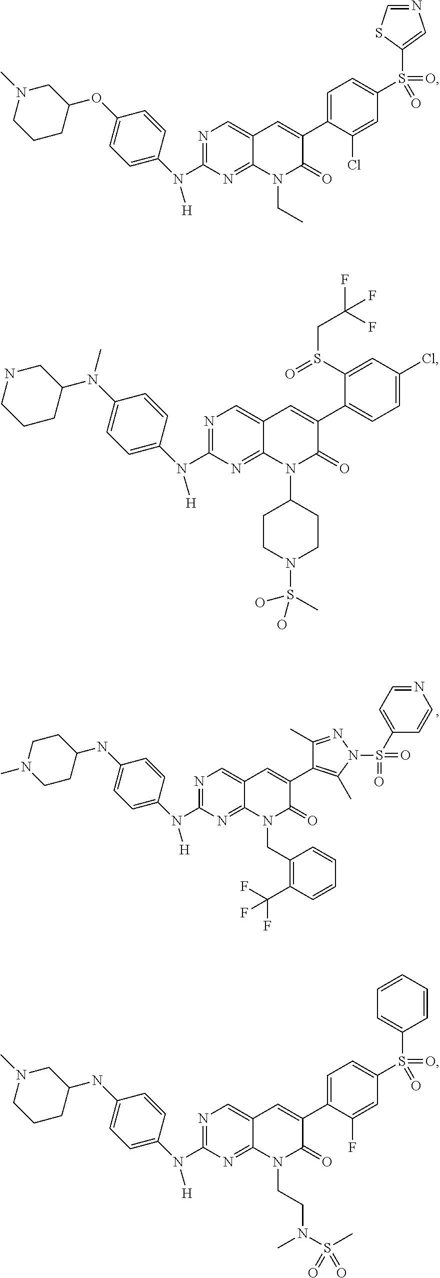 [SMILES: CCn1c(=O)c(-c2ccc(S(=O)(=O)c3cncs3)cc2Cl)cc2cnc(Nc3ccc(OC4CCCN(C)C4)cc3)nc21 ; CN(c1ccc(Nc2ncc3cc(-c4ccc(Cl)cc4S(=O)CC(F)(F)F)c(=O)n(C4CCN(S(C)(O)O)CC4)c3n2)cc1)C1CCCNC1 ; Cc1nn(S(=O)(=O)c2ccncc2)c(C)c1-c1cc2cnc(Nc3ccc(NC4CCN(C)CC4)cc3)nc2n(Cc2ccccc2C(F)(F)F)c1=O ; CN1CCCC(Nc2ccc(Nc3ncc4cc(-c5ccc(S(=O)(=O)c6ccccc6)cc5F)c(=O)n(CCN(C)S(C)(=O)=O)c4n3)cc2)C1]

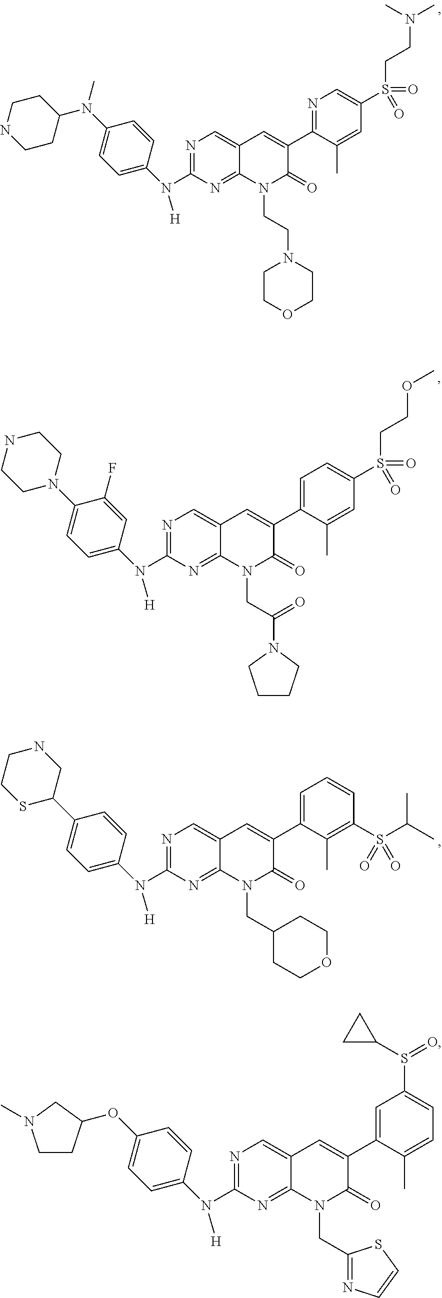

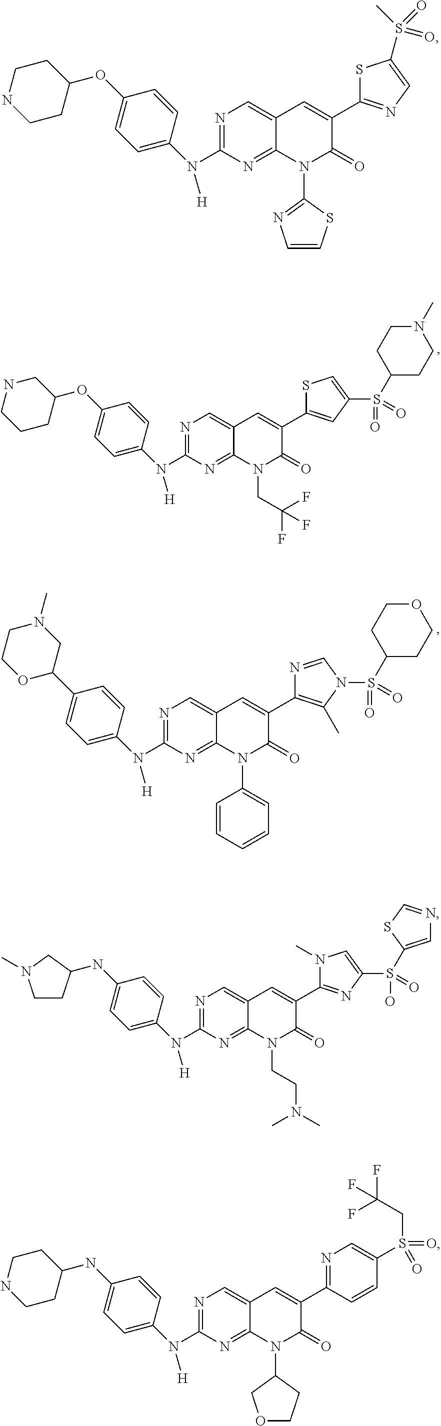

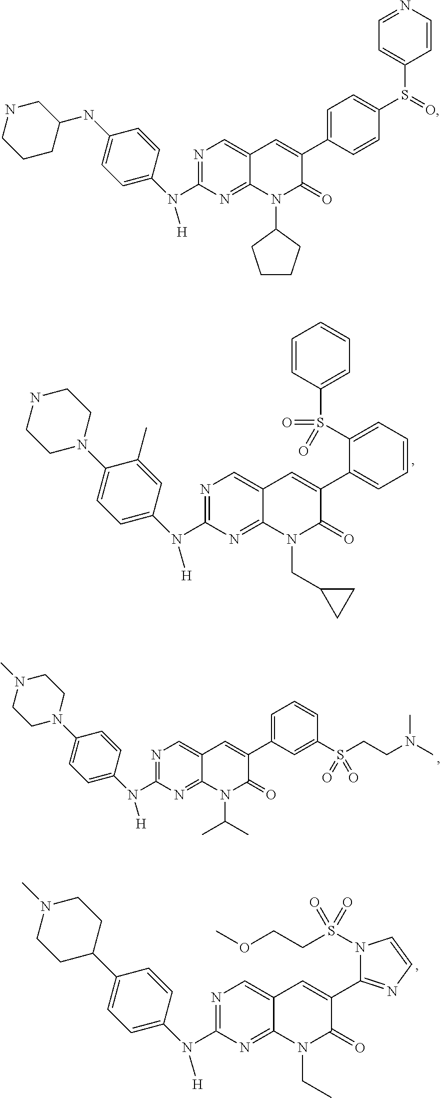

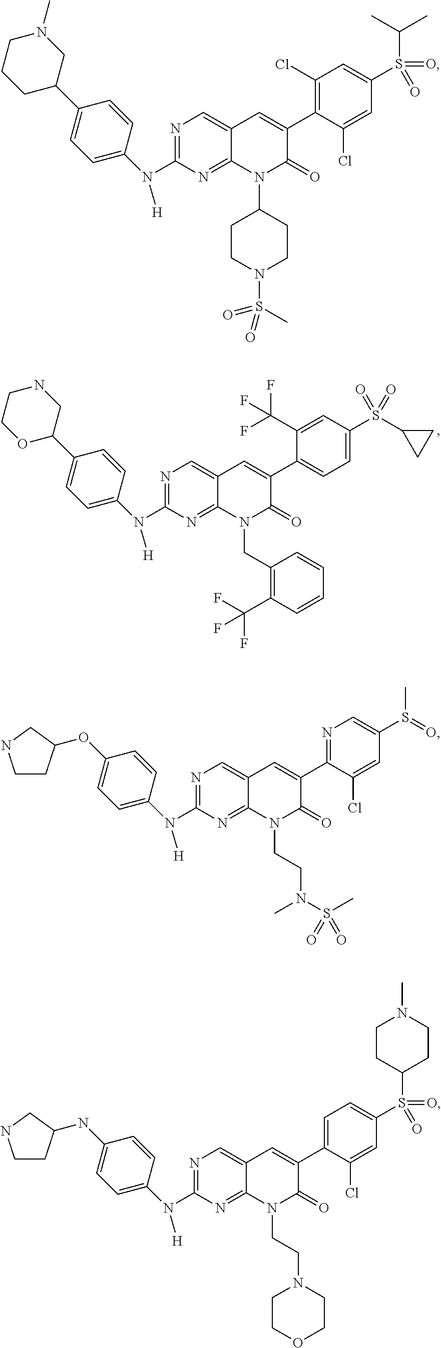 [SMILES: CC(C)S(=O)(=O)c1cc(Cl)c(-c2cc3cnc(Nc4ccc(C5CCCN(C)C5)cc4)nc3n(C3CCN(S(C)(=O)=O)CC3)c2=O)c(Cl)c1 ; O=c1c(-c2ccc(S(=O)(=O)C3CC3)cc2C(F)(F)F)cc2cnc(Nc3ccc(C4CNCCO4)cc3)nc2n1Cc1ccccc1C(F)(F)F ; CN(CCn1c(=O)c(-c2ncc(S(C)=O)cc2Cl)cc2cnc(Nc3ccc(OC4CCNC4)cc3)nc21)S(C)(=O)=O ; CN1CCC(S(=O)(=O)c2ccc(-c3cc4cnc(Nc5ccc(NC6CCNC6)cc5)nc4n(CCN4CCOCC4)c3=O)c(Cl)c2)CC1]

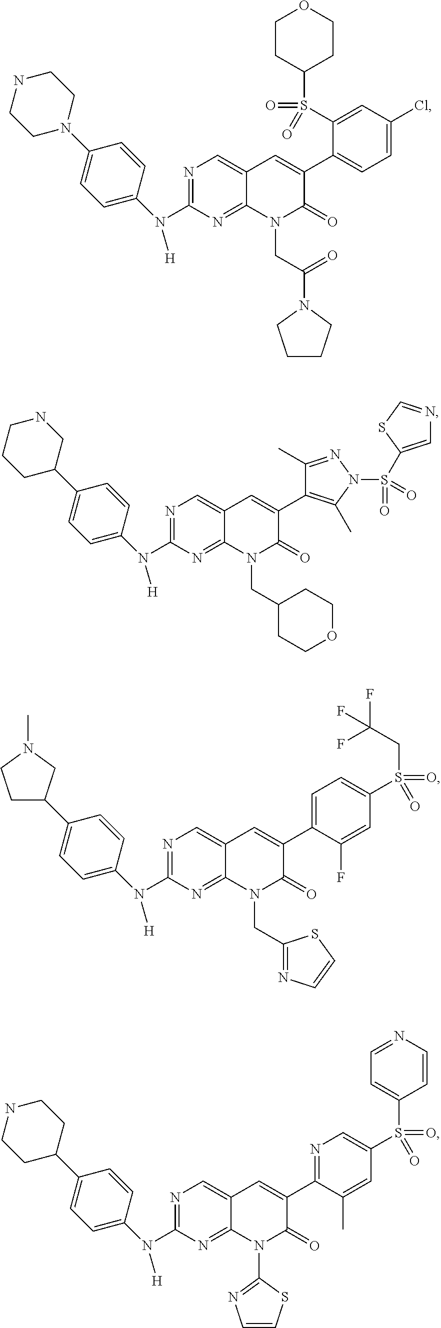 [SMILES: O=C(Cn1c(=O)c(-c2ccc(Cl)cc2S(=O)(=O)C2CCOCC2)cc2cnc(Nc3ccc(N4CCNCC4)cc3)nc21)N1CCCC1 ; Cc1nn(S(=O)(=O)c2cncs2)c(C)c1-c1cc2cnc(Nc3ccc(C4CCCNC4)cc3)nc2n(CC2CCOCC2)c1=O ; CN1CCC(c2ccc(Nc3ncc4cc(-c5ccc(S(=O)(=O)CC(F)(F)F)cc5F)c(=O)n(Cc5nccs5)c4n3)cc2)C1 ; Cc1cc(S(=O)(=O)c2ccncc2)cnc1-c1cc2cnc(Nc3ccc(C4CCNCC4)cc3)nc2n(-c2nccs2)c1=O]

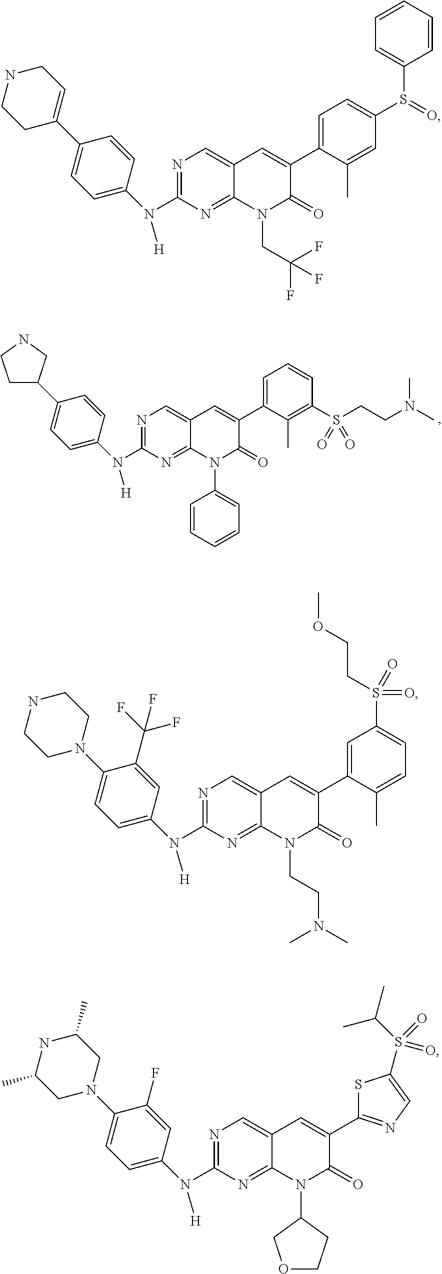

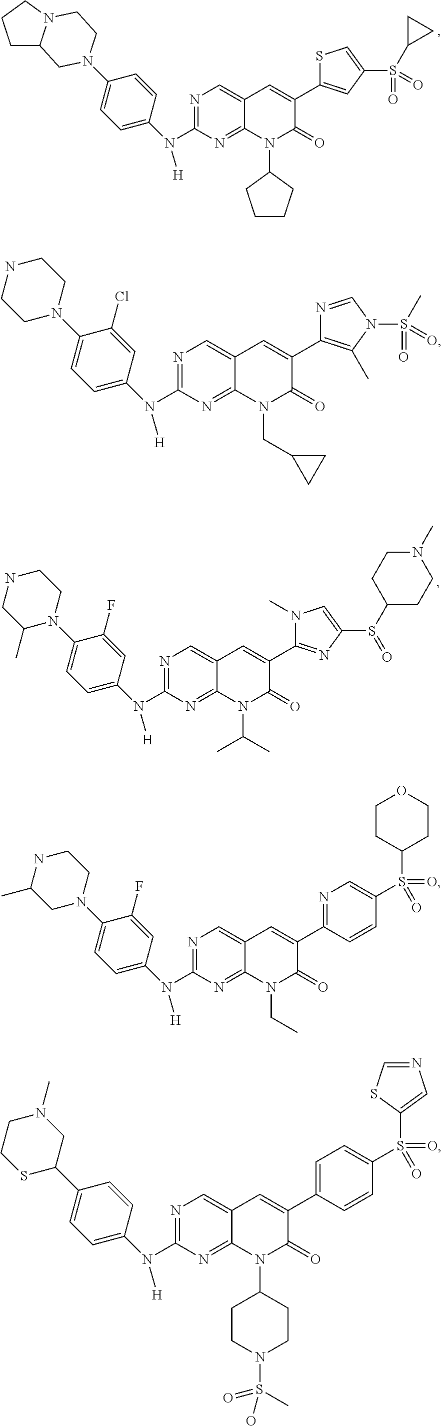

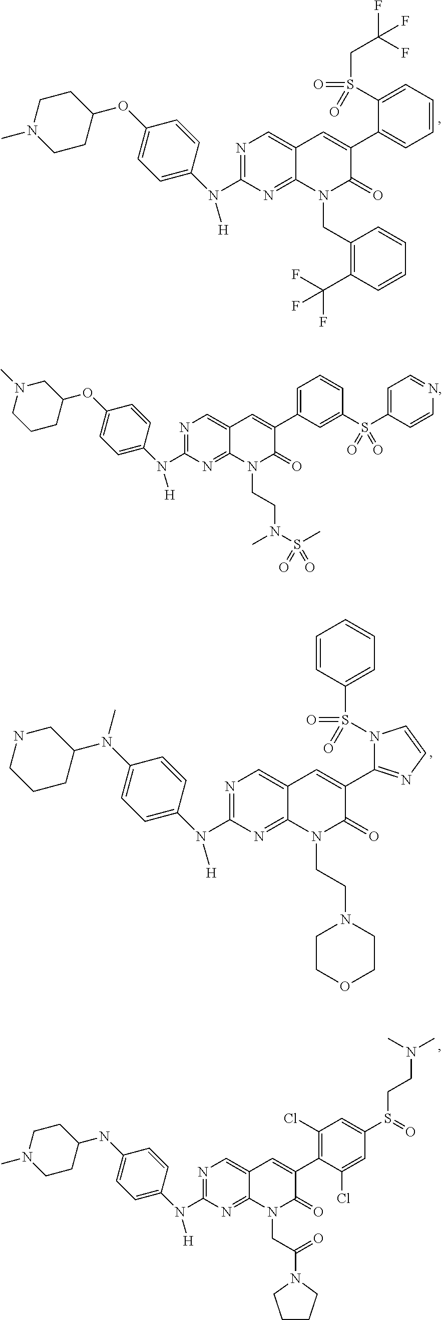

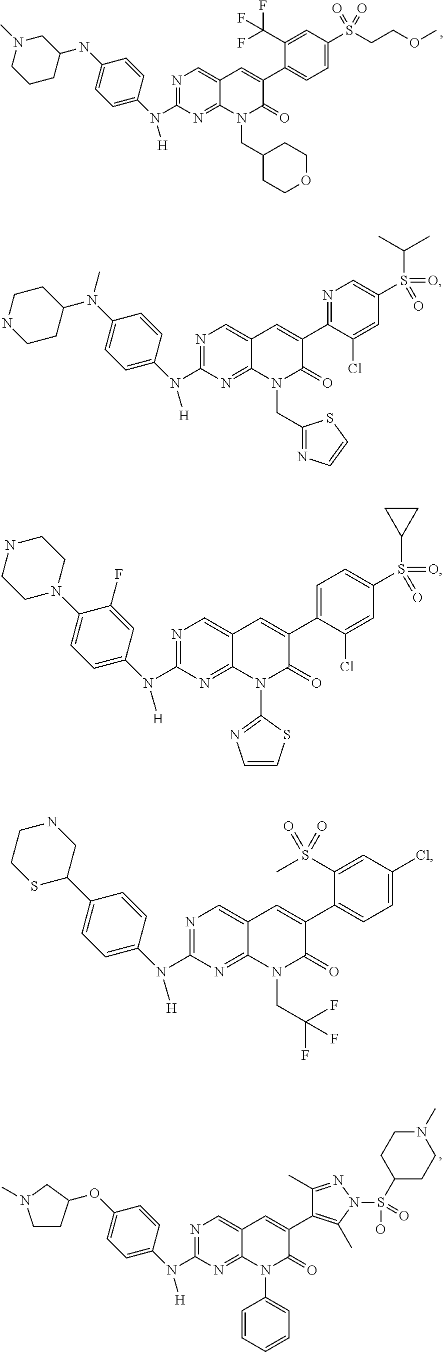

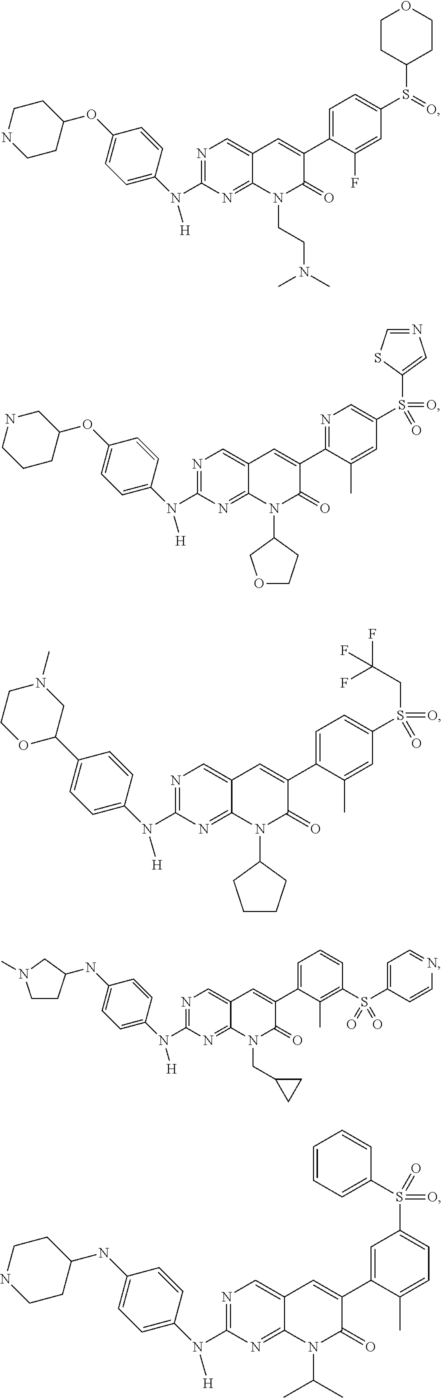 [SMILES: CN(C)CCn1c(=O)c(-c2ccc(S(=O)C3CCOCC3)cc2F)cc2cnc(Nc3ccc(OC4CCNCC4)cc3)nc21 ; Cc1cc(S(=O)(=O)c2cncs2)cnc1-c1cc2cnc(Nc3ccc(OC4CCCNC4)cc3)nc2n(C2CCOC2)c1=O ; Cc1cc(S(=O)(=O)CC(F)(F)F)ccc1-c1cc2cnc(Nc3ccc(C4CN(C)CCO4)cc3)nc2n(C2CCCC2)c1=O ; Cc1c(-c2cc3cnc(Nc4ccc(NC5CCN(C)C5)cc4)nc3n(CC3CC3)c2=O)cccc1S(=O)(=O)c1ccncc1 ; Cc1ccc(S(=O)(=O)c2ccccc2)cc1-c1cc2cnc(Nc3ccc(NC4CCNCC4)cc3)nc2n(C(C)C)c1=O]

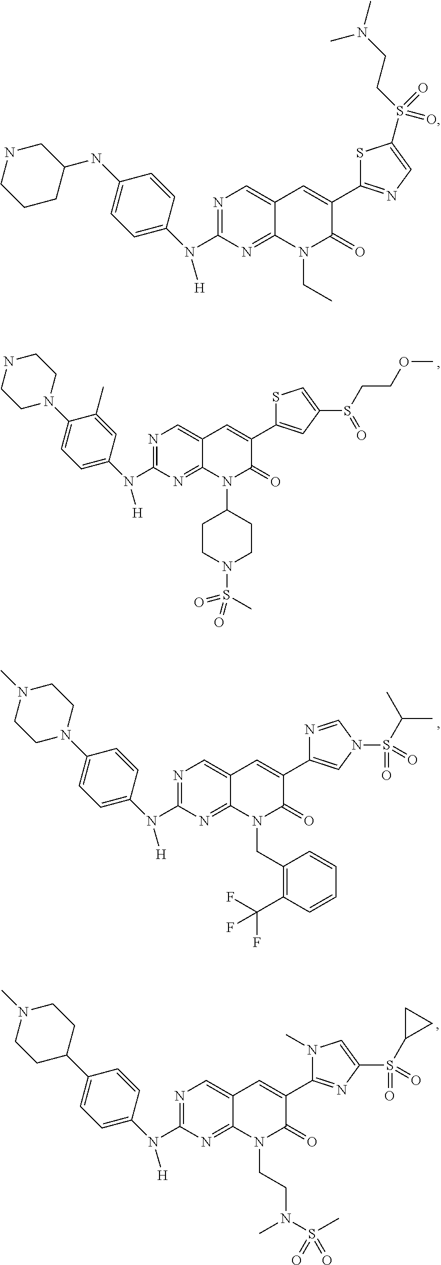

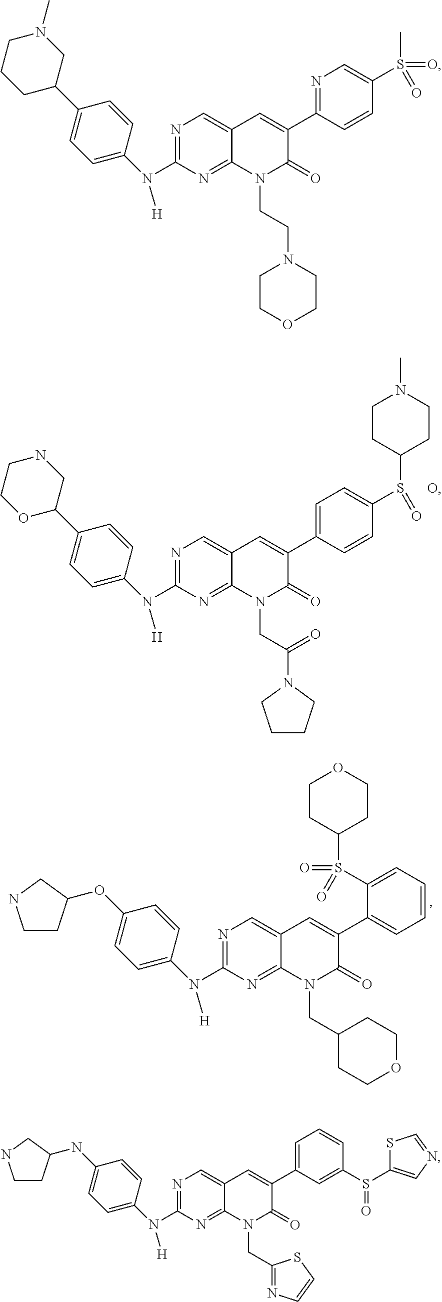

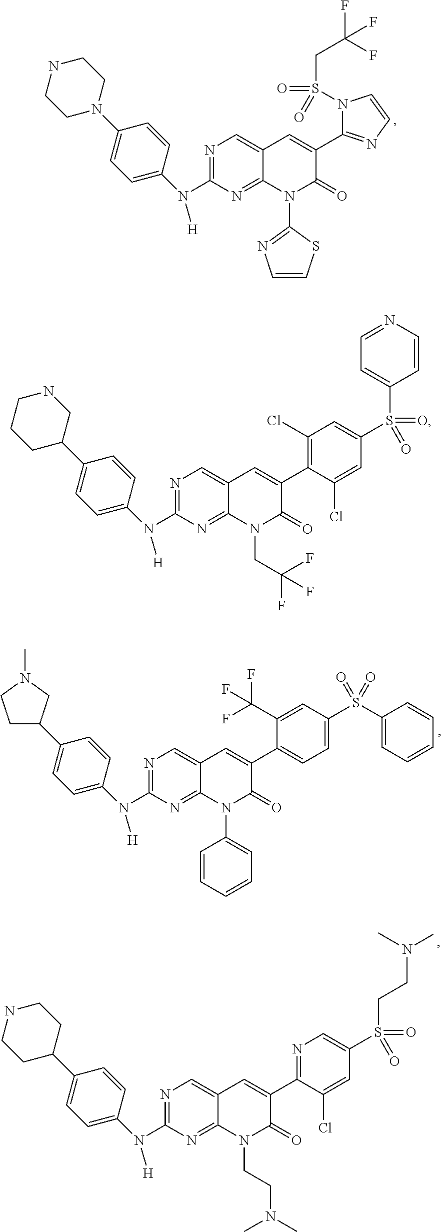 [SMILES: O=c1c(-c2nccn2S(=O)(=O)CC(F)(F)F)cc2cnc(Nc3ccc(N4CCNCC4)cc3)nc2n1-c1nccs1 ; O=c1c(-c2c(Cl)cc(S(=O)(=O)c3ccncc3)cc2Cl)cc2cnc(Nc3ccc(C4CCCNC4)cc3)nc2n1CC(F)(F)F ; CN1CCC(c2ccc(Nc3ncc4cc(-c5ccc(S(=O)(=O)c6ccccc6)cc5C(F)(F)F)c(=O)n(-c5ccccc5)c4n3)cc2)C1 ; CN(C)CCn1c(=O)c(-c2ncc(S(=O)(=O)CCN(C)C)cc2Cl)cc2cnc(Nc3ccc(C4CCNCC4)cc3)nc21]

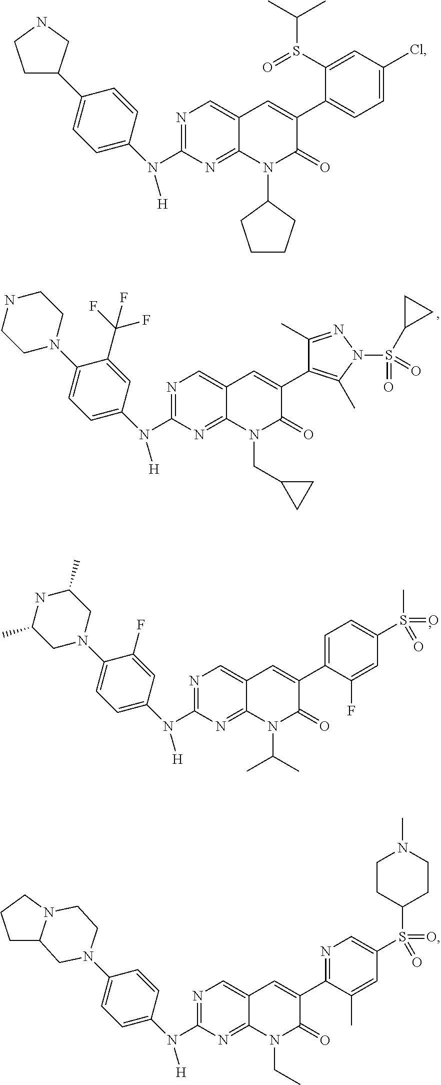 [SMILES: CC(C)S(=O)c1cc(Cl)ccc1-c1cc2cnc(Nc3ccc(C4CCNC4)cc3)nc2n(C2CCCC2)c1=O ; Cc1nn(S(=O)(=O)C2CC2)c(C)c1-c1cc2cnc(Nc3ccc(N4CCNCC4)c(C(F)(F)F)c3)nc2n(CC2CC2)c1=O ; CC(C)n1c(=O)c(-c2ccc(S(C)(=O)=O)cc2F)cc2cnc(Nc3ccc(N4C[C@@H](C)N[C@@H](C)C4)c(F)c3)nc21 ; CCn1c(=O)c(-c2ncc(S(=O)(=O)C3CCN(C)CC3)cc2C)cc2cnc(Nc3ccc(N4CCN5CCCC5C4)cc3)nc21]

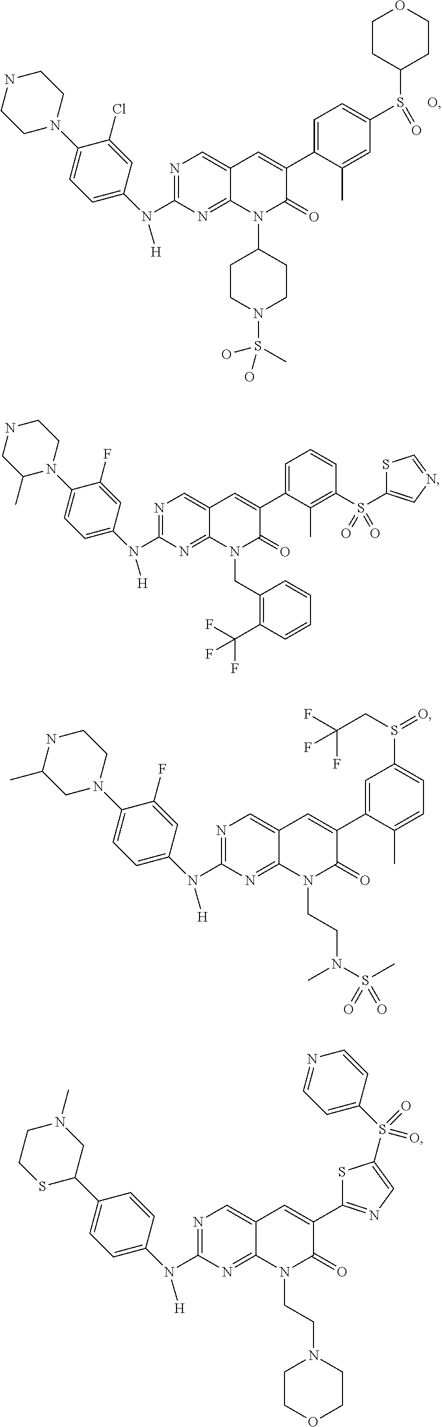

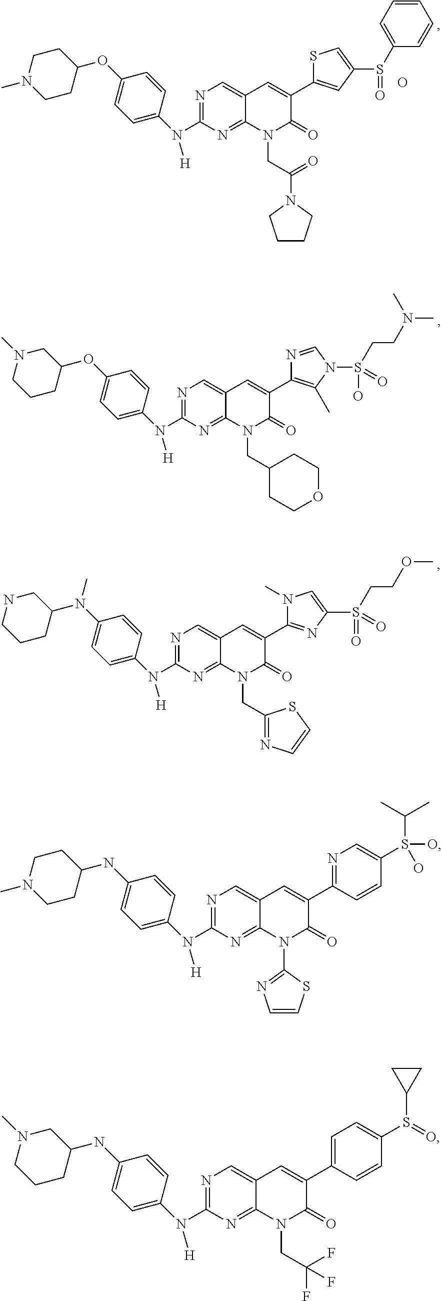

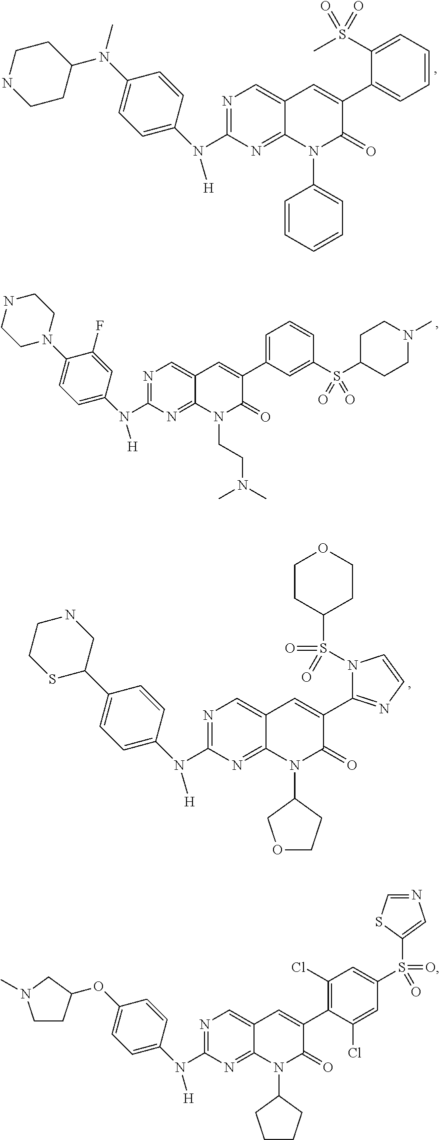

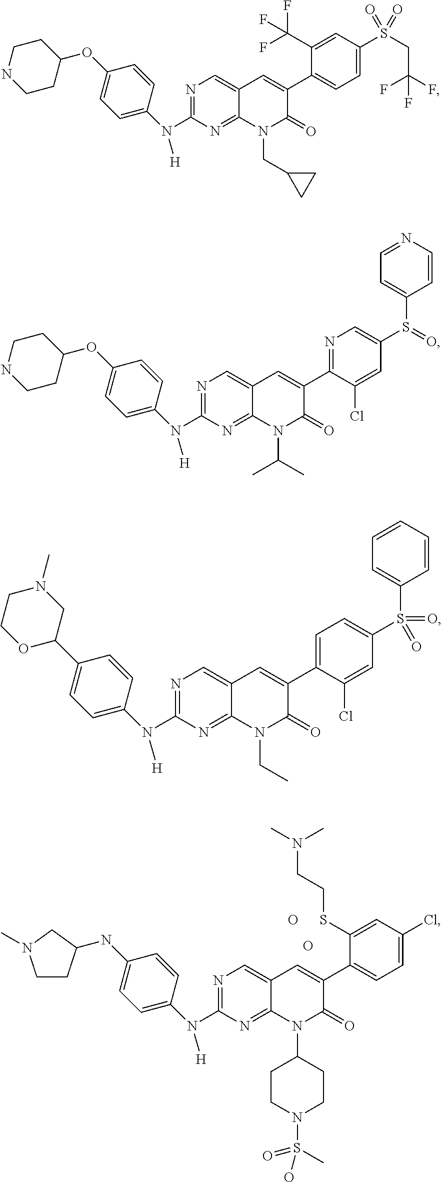

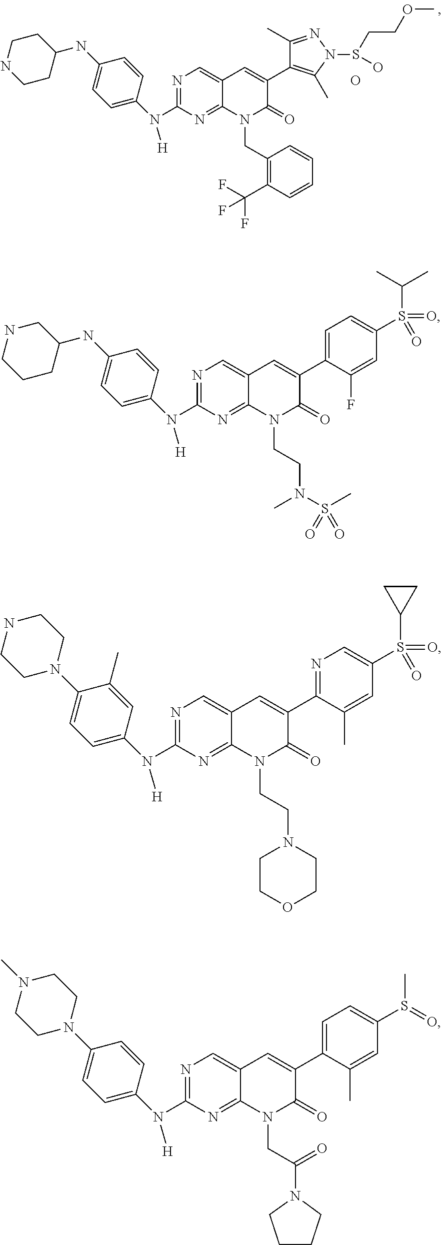

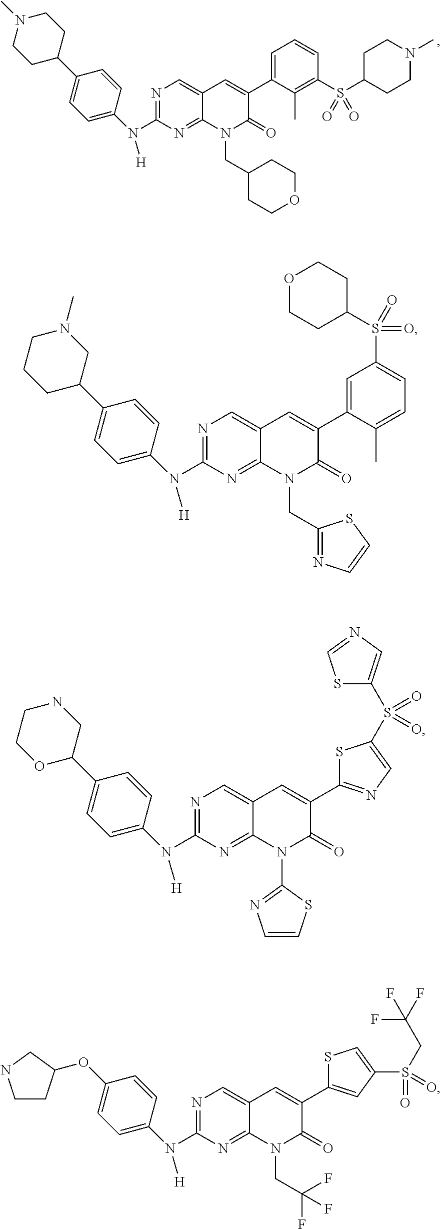

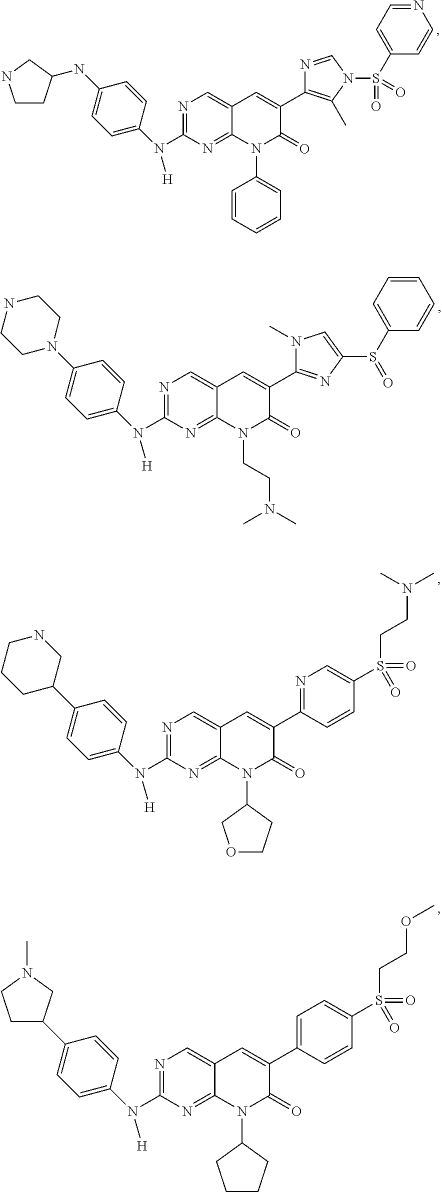 [SMILES: Cc1c(-c2cc3cnc(Nc4ccc(NC5CCNC5)cc4)nc3n(-c3ccccc3)c2=O)ncn1S(=O)(=O)c1ccncc1 ; CN(C)CCn1c(=O)c(-c2nc(S(=O)c3ccccc3)cn2C)cc2cnc(Nc3ccc(N4CCNCC4)cc3)nc21 ; CN(C)CCS(=O)(=O)c1ccc(-c2cc3cnc(Nc4ccc(C5CCCNC5)cc4)nc3n(C3CCOC3)c2=O)nc1 ; COCCS(=O)(=O)c1ccc(-c2cc3cnc(Nc4ccc(C5CCN(C)C5)cc4)nc3n(C3CCCC3)c2=O)cc1]

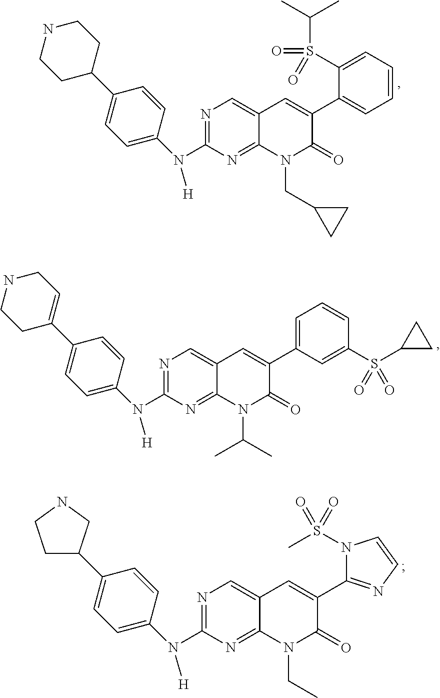 [SMILES: CC(C)S(=O)(=O)c1ccccc1-c1cc2cnc(Nc3ccc(C4CCNCC4)cc3)nc2n(CC2CC2)c1=O ; CC(C)n1c(=O)c(-c2cccc(S(=O)(=O)C3CC3)c2)cc2cnc(Nc3ccc(C4=CCNCC4)cc3)nc21 ; CCn1c(=O)c(-c2nccn2S(C)(=O)=O)cc2cnc(Nc3ccc(C4CCNC4)cc3)nc21]

or a pharmaceutically acceptable salt, solvate, or N-oxide thereof.

In another embodiment are PAK inhibitors suitable for treating cancer having the structure:

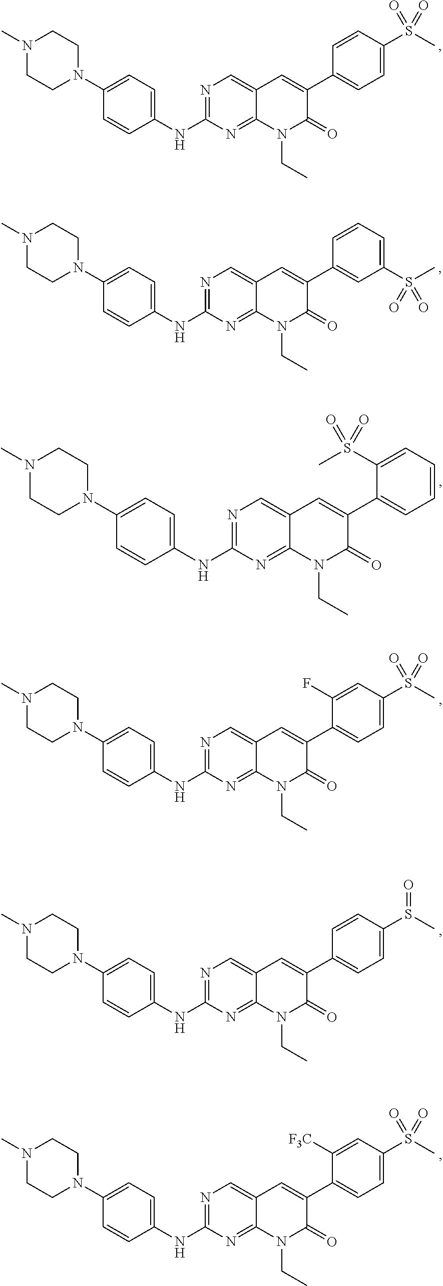

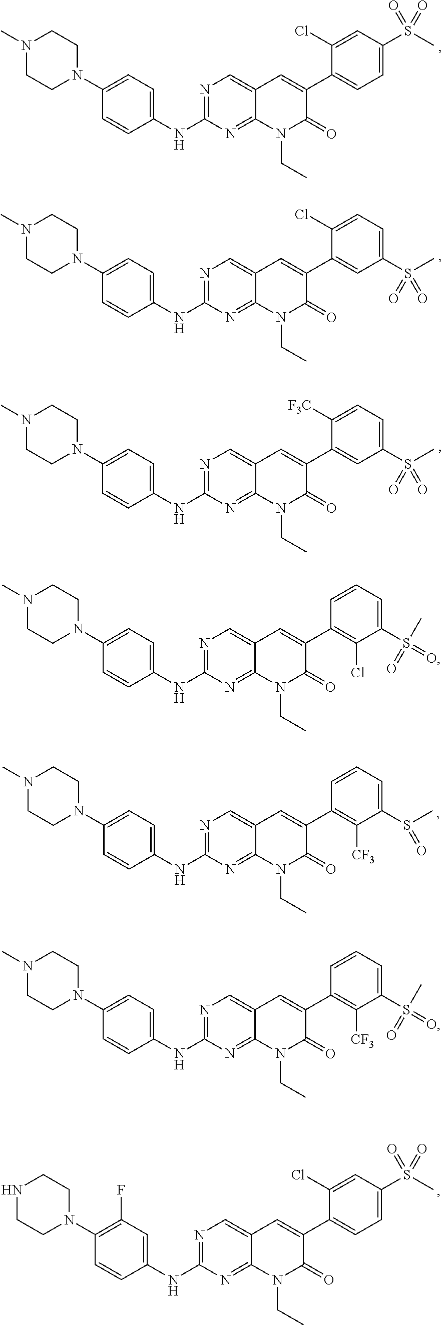

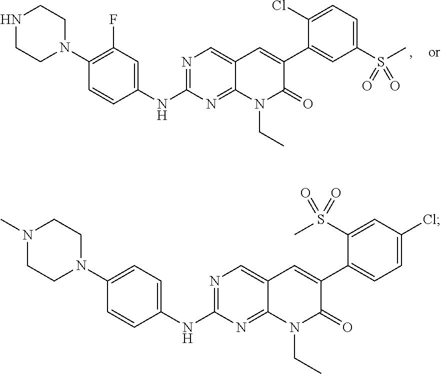

or a pharmaceutically acceptable salt, solvate or N-oxide thereof.

In another embodiment are PAK inhibitors suitable for treating cancer having the structure of Formula VI or a pharmaceutically acceptable salt, solvate or N-oxide thereof wherein:

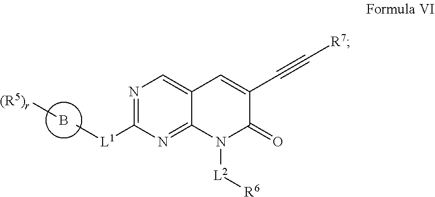

wherein:
L¹ is O, NR⁸, or S;
ring B is an optionally substituted aryl or heteroaryl;
R⁷ is H, OR¹⁰, N(R¹⁰)₂, a substituted or unsubstituted alkyl, substituted or unsubstituted cycloalkyl, substituted or unsubstituted heterocycloalkyl, substituted or unsubstituted aryl or substituted or unsubstituted heteroaryl;
L² is C(R¹R²)ₚ, O, NR⁸, or S and R⁶ is alkyl, cycloalkyl, heterocycloalkyl, aryl or heteroaryl optionally substituted with at least one R¹²; or
L² is a bond and R⁶ is alkyl, cycloalkyl, aryl or heteroaryl optionally substituted with at least one R¹²;
R¹² is halogen, —CN, —NO₂, —OH, —OCF₃, —OCHF₂, —OCF₂H, —CF₃, —S(═O)R⁹, —S(═O)₂R⁹, —NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —OR¹⁰, —NR¹⁰C(═O)R¹⁰—NR¹⁰C(═O)OR¹⁰, —NR¹⁰C(═O)N(R¹⁰)₂, substituted or unsubstituted alkyl, substituted or unsubstituted alkoxy, substituted or unsubstituted heteroalkyl, substituted or unsubstituted cycloalkyl or substituted or unsubstituted heterocycloalkyl; substituted or unsubstituted aryl or substituted or unsubstituted heteroaryl;
each R⁵ is independently halogen, —CN, —NO₂, —OH, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, substituted or unsubstituted alkyl, substituted or unsubstituted alkoxy, substituted or unsubstituted heteroalkyl, substituted or unsubstituted cycloalkyl or substituted or unsubstituted heterocycloalkyl;
R⁸ is H or substituted or unsubstituted alkyl, substituted or unsubstituted cycloalkyl, substituted or unsubstituted heterocycloalkyl, substituted or unsubstituted aryl or substituted or unsubstituted heteroaryl;
R⁹ is substituted or unsubstituted alkyl, substituted or unsubstituted cycloalkyl, substituted or unsubstituted heterocycloalkyl, substituted or unsubstituted aryl or substituted or unsubstituted heteroaryl;
each R¹⁰ is independently H, substituted or unsubstituted alkyl, substituted or unsubstituted cycloalkyl, substituted or unsubstituted heterocycloalkyl substituted or unsubstituted aryl or substituted or unsubstituted heteroaryl, or two R¹⁰ together with the atoms to which they are attached form a substituted or unsubstituted heterocycle;
p is 1, 2 or 3; and
r is 0-8.

In one embodiment is a compound of Formula VI wherein R⁷ is H. In one embodiment, R⁷ is OH. In another embodiment, R⁷ is N(R¹⁰)₂. In another embodiment is a compound of Formula VI wherein R⁷ is an unsubstituted alkyl. In yet another embodiment is a compound of Formula VI wherein unsubstituted alkyl is selected from methyl, ethyl, n-propyl, iso-propyl, n-butyl, iso-butyl and tert-butyl. In another embodiment, R⁷ is substituted or unsubstituted cycloalkyl. In one embodiment, R⁷ is substituted or unsubstituted heteroalkyl. In a further embodiment, R⁷ is substituted or unsubstituted heterocycloalkyl. In yet a further embodiment, R⁷ is substituted or unsubstituted aryl. In a further embodiment, R⁷ is substituted or unsubstituted heteroaryl.

In yet another embodiment is a compound of Formula VI wherein R⁷ is selected from:

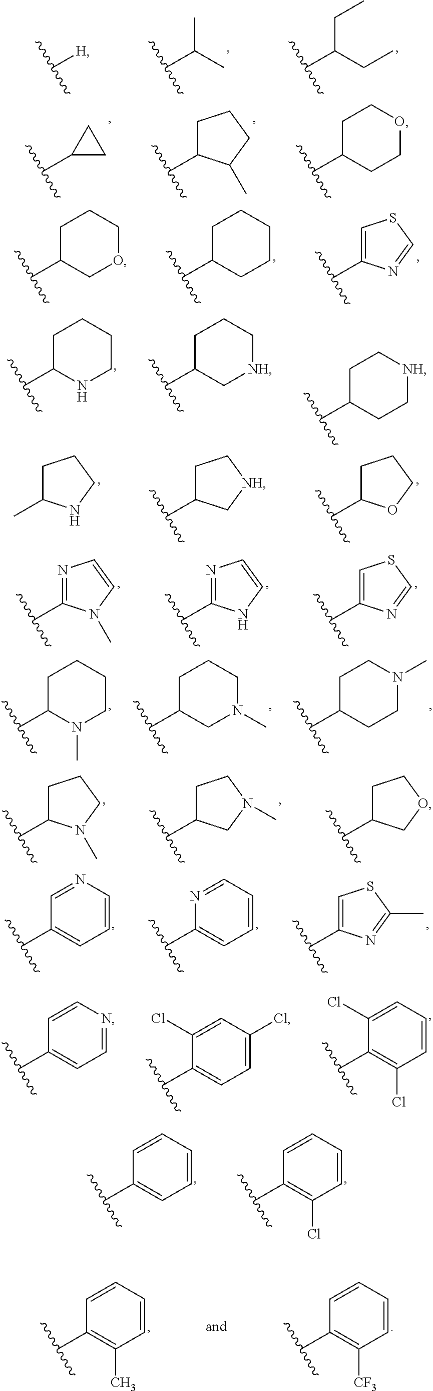

In a further embodiment is a compound of Formula VI wherein L¹ is NR⁸. In yet a further embodiment R⁸ is H. In another embodiment, L¹ is O. In a further embodiment, L¹ is S.

In another embodiment L² is a bond and R⁶ is a substituted or unsubstituted aryl. In yet another embodiment R⁶ is an unsubstituted phenyl. In a further embodiment is a compound of Formula VI wherein R⁶ is a substituted phenyl. In yet a further embodiment is a compound of Formula VI wherein L² is a bond and R⁶ is aryl.

In one embodiment L² is a bond and R⁶ is heteroaryl. In a further embodiment, heteroaryl is selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine and pyrazine.

In another embodiment, R⁶ is heterocycloalkyl. In a further embodiment, heterocycloalkyl is selected from morpholino, piperidinyl, tetrahydropyran, tetrahydrofuranyl, pyrrolidinyl, or piperazinyl.

In another embodiment L² is CH₂ and R⁶ is aryl. In yet another embodiment L² is CH₂ and R⁶ is heteroaryl. In a further embodiment L² is CH₂ and R⁶ is alkyl. In yet a further embodiment L² is CH₂ and R⁶ is cycloalkyl. In one embodiment is a compound of Formula VI wherein R⁶ is substituted with at least one R¹² selected from halogen, alkyl, cycloalkyl, heterocycloalkyl, aryl or heteroaryl. In another embodiment R¹² is F, Cl, Br, or I. In yet another embodiment R¹² is alkyl selected from methyl, ethyl, n-propyl, iso-propyl, n-butyl, iso-butyl, or tert-butyl.

In one embodiment is a compound of Formula VI wherein R¹² is cyclopropyl.

In another embodiment is a compound of Formula VI wherein R¹² is cyclopentyl.

In a further embodiment is a compound of Formula VI wherein R¹² is phenyl.

In one embodiment is a compound of Formula VI wherein R¹² is pyrrolidine or piperidine.

In yet a further embodiment is a compound of Formula VI wherein R¹² is thiazole.

In one embodiment is a compound of Formula VI wherein ring B is a substituted or unsubstituted aryl. In another embodiment is a compound of Formula VI wherein ring B is an unsubstituted phenyl. In yet another embodiment is a compound of Formula VI wherein ring B is a substituted phenyl.

In a further embodiment, ring B is a substituted or unsubstituted naphthalene. In a further embodiment, is a compound of Formula VI, wherein ring B is a heteroaryl ring selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, and pyrazine.

In another embodiment is a compound of Formula VI wherein ring B is

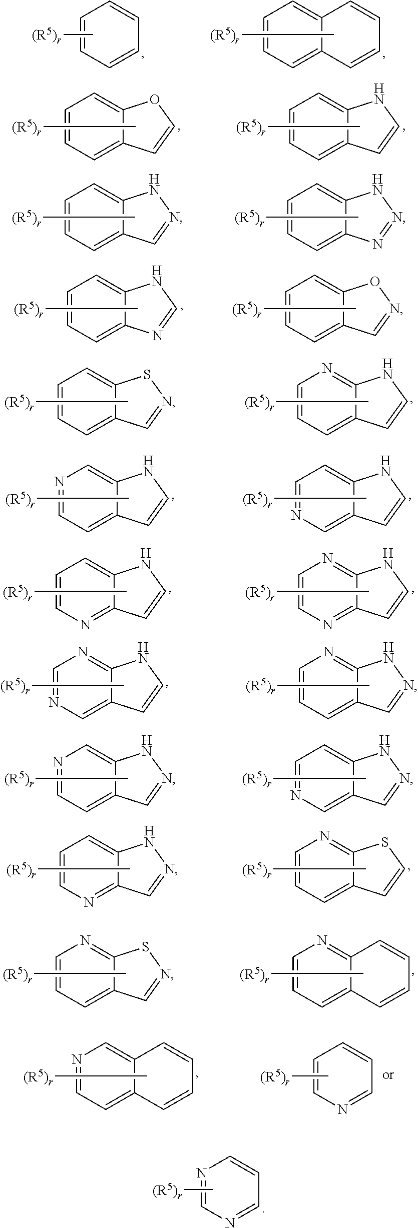

In one embodiment, is a compound of Formula VI, wherein R⁵ is halogen, —CN, —OH, substituted or unsubstituted alkyl, —OR¹⁰, NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═)OR¹⁰, —NR¹⁰C(═O)N(R¹⁰)₂, or substituted or unsubstituted heterocycloalkyl. In one embodiment, R⁵ is selected from F, Cl, Br, or I. In another embodiment R⁵ is F.

In another embodiment, is a compound of Formula VI, wherein at least one R⁵ is —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In one embodiment, is a compound of Formula VI, wherein at least one R⁵ is —N(R¹⁰)₂, or substituted or unsubstituted heterocycloalkyl. In yet another embodiment, is a compound of Formula VI wherein at least one of R⁵ is a substituted or unsubstituted piperazine, substituted or unsubstituted piperidine, substituted or unsubstituted pyrrolidine or substituted or unsubstituted morpholine. In a further embodiment, is a compound of Formula VI, wherein at least one R⁵ is —OR¹⁰. In one embodiment is a compound of Formula VI, wherein at least one R⁵ is —OR¹⁰ and R¹⁰ is H. In another embodiment, R¹⁰ is alkyl selected from methyl, ethyl, n-propyl, iso-propyl, n-butyl, iso-butyl, and tert-butyl.

In another embodiment, is a compound of Formula VI wherein R⁵ is a C₃-C₆cycloalkyl ring. In another embodiment, R⁵ is cyclopropyl. In another embodiment, R⁵ is cyclopentyl. In another embodiment, R⁵ is cyclohexyl.

In another embodiment, is a compound of Formula VI wherein R⁵ is a substituted or unsubstituted heterocycloalkyl ring. In another embodiment, R⁵ is a substituted or unsubstituted piperazine. In another embodiment, R⁵ is an unsubstituted piperazine. In another embodiment, R⁵ is N-methylpiperazine. In another embodiment, R⁵ is a substituted or unsubstituted piperidine. In another embodiment, R⁵ is an unsubstituted piperidine.

In another embodiment is a compound of Formula VI wherein r is 0. In another embodiment, r is 1. In a further embodiment, r is 2. In another embodiment is a compound of Formula VI wherein r is 1; and R⁵ is substituted or unsubstituted piperazine.

In one embodiment is a compound of Formula VI wherein ring B is substituted with —N(R¹⁰)₂, wherein R¹⁰ is each independently selected from H and a substituted or unsubstituted heterocycloalkyl. In another embodiment is a compound of Formula VI wherein ring B is substituted with —NHR¹⁰ wherein R¹⁰ is a substituted or unsubstituted piperazine, substituted or unsubstituted piperidine, substituted or unsubstituted pyrrolidine or substituted or unsubstituted morpholine. In a further embodiment is a compound of Formula VI wherein ring B is substituted with —N(CH₃)R¹⁰ wherein R¹⁰ is a substituted or unsubstituted piperazine, substituted or unsubstituted piperidine, substituted or unsubstituted pyrrolidine or substituted or unsubstituted morpholine.

Also presented herein is a compound of Formula VI wherein ring B is substituted with —OR¹⁰ wherein R¹⁰ is a substituted or unsubstituted heterocycloalkyl. In another embodiment is a compound of Formula VI wherein ring B is substituted with —OR¹⁰ wherein R¹⁰ is a substituted or unsubstituted piperazine, substituted or unsubstituted piperidine, substituted or unsubstituted pyrrolidine or substituted or unsubstituted morpholine. In yet another embodiment is a compound of Formula VI wherein ring B is substituted with at least one CF₃.

In yet another embodiment, ring B is substituted with at least two R⁵. In another embodiment, ring B is substituted with halogen and a substituted or unsubstituted heterocycloalkyl. In another embodiment, ring B is substituted with at least one F, Cl, Br, or I and a substituted or unsubstituted piperazine, substituted or unsubstituted piperidine, substituted or unsubstituted pyrrolidine, or substituted or unsubstituted morpholine.

In one embodiment is a compound of Formula VI wherein

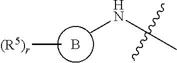

is selected from:

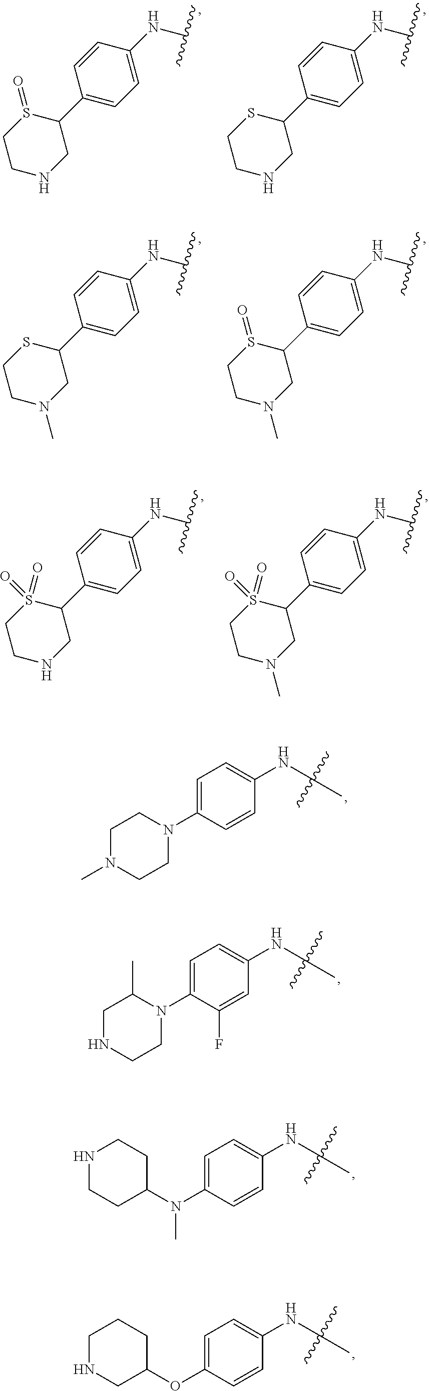

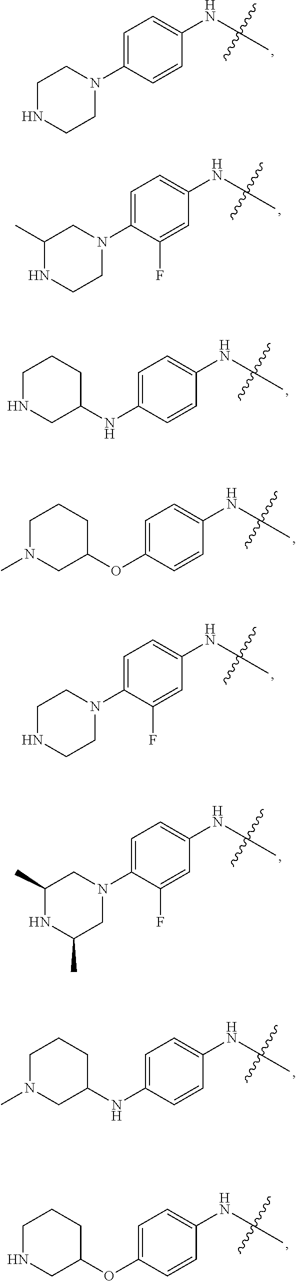

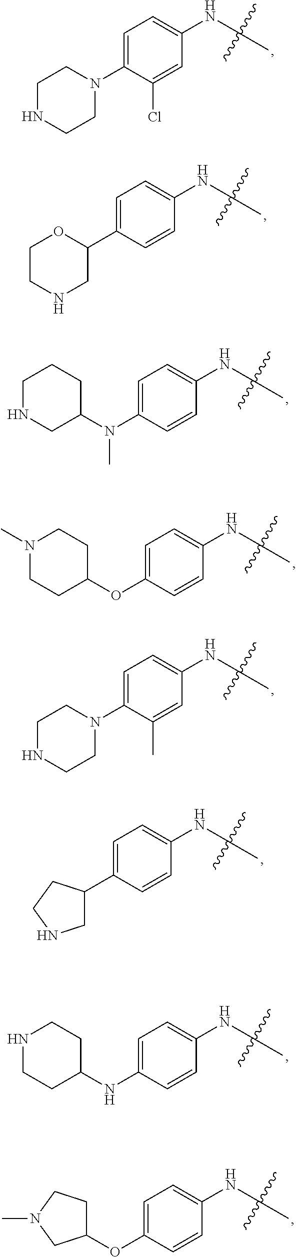

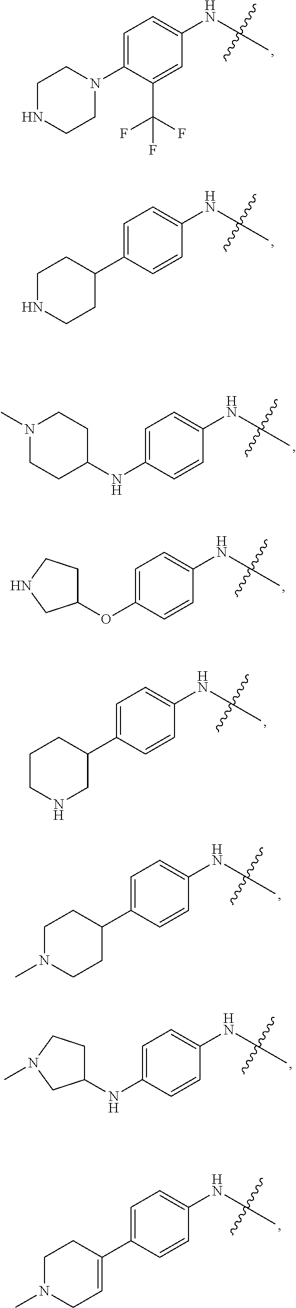

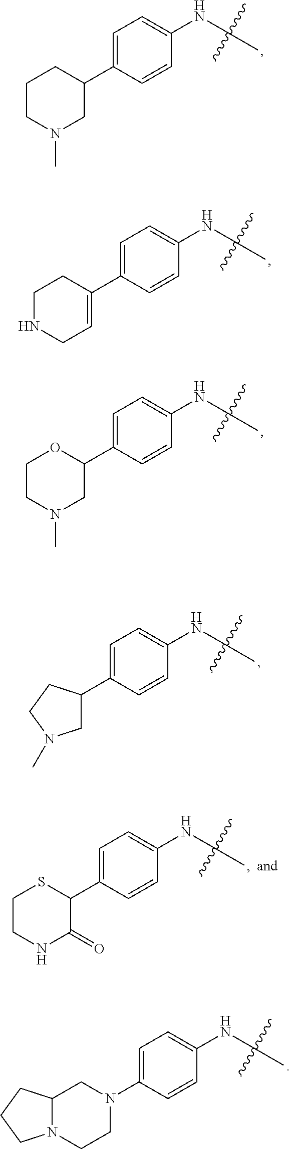 [SMILES: *Nc1ccc(C2CCCN(C)C2)cc1 ; *Nc1ccc(C2=CCNCC2)cc1 ; *Nc1ccc(C2CN(C)CCO2)cc1 ; *Nc1ccc(C2CCN(C)C2)cc1 ; *Nc1ccc(C2SCCNC2=O)cc1 ; *Nc1ccc(N2CCN3CCCC3C2)cc1]

In yet another embodiment is a compound of Formula VI wherein

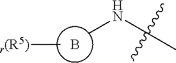

is selected from:

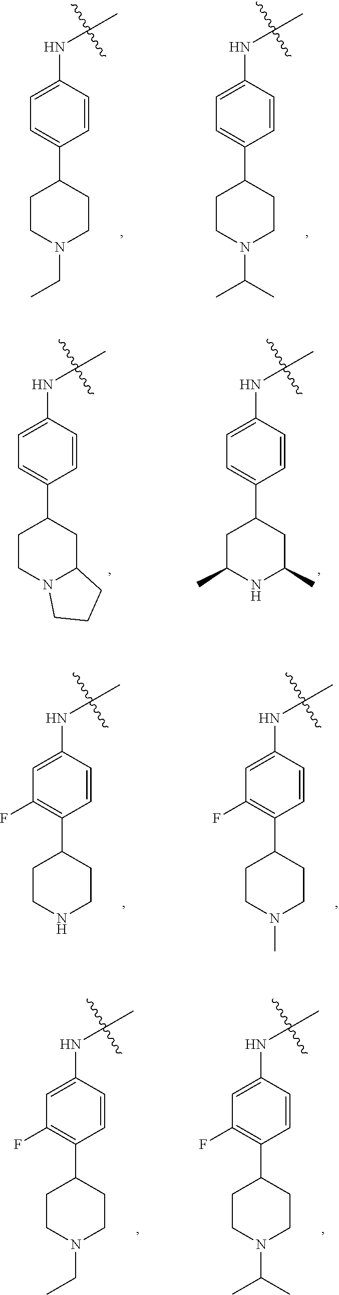

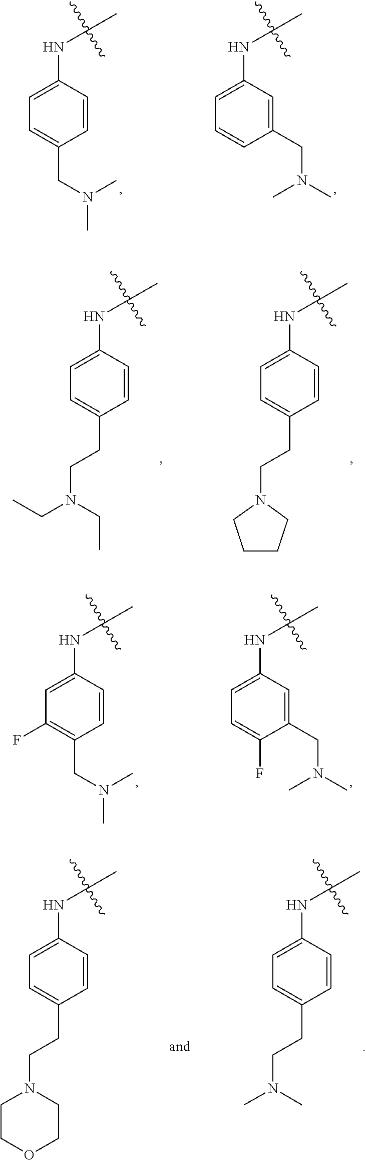

In one embodiment, is a compound of Formula VI, wherein R⁵ is halogen, —CN, —OH, substituted or unsubstituted alkyl, —OR¹⁰, —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In one embodiment, R⁵ is selected from F, Cl, Br, or I. In another embodiment R⁵ is F.

In another embodiment, is a compound of Formula VI wherein at least one R⁵ is —NR¹⁰S(═O)₂R⁹, —S(═O)₂N(R¹⁰)₂, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, or substituted or unsubstituted heterocycloalkyl. In one embodiment, is a compound of Formula VI wherein at least one R⁵ is)-N(R¹⁰)₂, or substituted or unsubstituted heterocycloalkyl. In yet another embodiment, is a compound of Formula VI wherein at least one of R⁵ is a substituted or unsubstituted piperazine, substituted or unsubstituted piperidine, substituted or unsubstituted pyrrolidine or substituted or unsubstituted morpholine. In a further embodiment, is a compound of Formula VI wherein at least one R⁵ is —OR¹⁰. In one embodiment is a compound of Formula VI wherein at least one R⁵ is —OR¹⁰ and R¹⁰ is H. In another embodiment, R¹⁰ is alkyl selected from methyl, ethyl, n-propyl, iso-propyl, n-butyl, iso-butyl, and tert-butyl.

In one embodiment is a compound of Formula VI wherein ring B is substituted with —N(R¹⁰)₂, wherein R¹⁰ is each independently selected from H and a substituted or unsubstituted heterocycloalkyl. In another embodiment is a compound of Formula VI wherein ring B is substituted with —NHR¹⁰ wherein R¹⁰ is a substituted or unsubstituted piperazine, substituted or unsubstituted piperidine, substituted or unsubstituted pyrrolidine or substituted or unsubstituted morpholine. In a further embodiment is a compound of Formula VI wherein ring B is substituted with —N(CH₃)R¹⁰ wherein R¹⁰ is a substituted or unsubstituted piperazine, substituted or unsubstituted piperidine, substituted or unsubstituted pyrrolidine or substituted or unsubstituted morpholine.

Also presented herein is a compound of Formula VI wherein ring B is substituted with —OR¹⁰ wherein R¹⁰ is a substituted or unsubstituted heterocycloalkyl. In another embodiment is a compound of Formula VI wherein ring B is substituted with —OR¹⁰ wherein R¹⁰ is a substituted or unsubstituted piperazine, substituted or unsubstituted piperidine, substituted or unsubstituted pyrrolidine or substituted or unsubstituted morpholine. In yet another embodiment is a compound of Formula VI wherein ring B is substituted with at least one CF₃.

In yet another embodiment, ring B is substituted with at least two R⁵. In another embodiment, ring B is substituted with halogen and a substituted or unsubstituted heterocycloalkyl. In another embodiment, ring B is substituted with at least one F, Cl, Br, or I and a substituted or unsubstituted piperazine, substituted or unsubstituted piperidine, substituted or unsubstituted pyrrolidine, or substituted or unsubstituted morpholine.

In another embodiment is a compound having the structure:

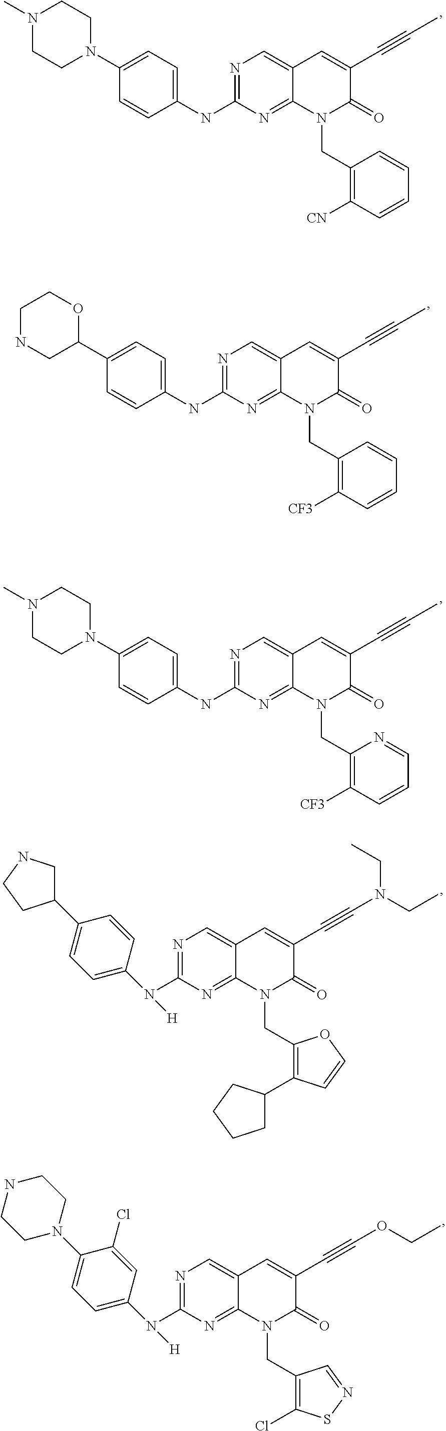

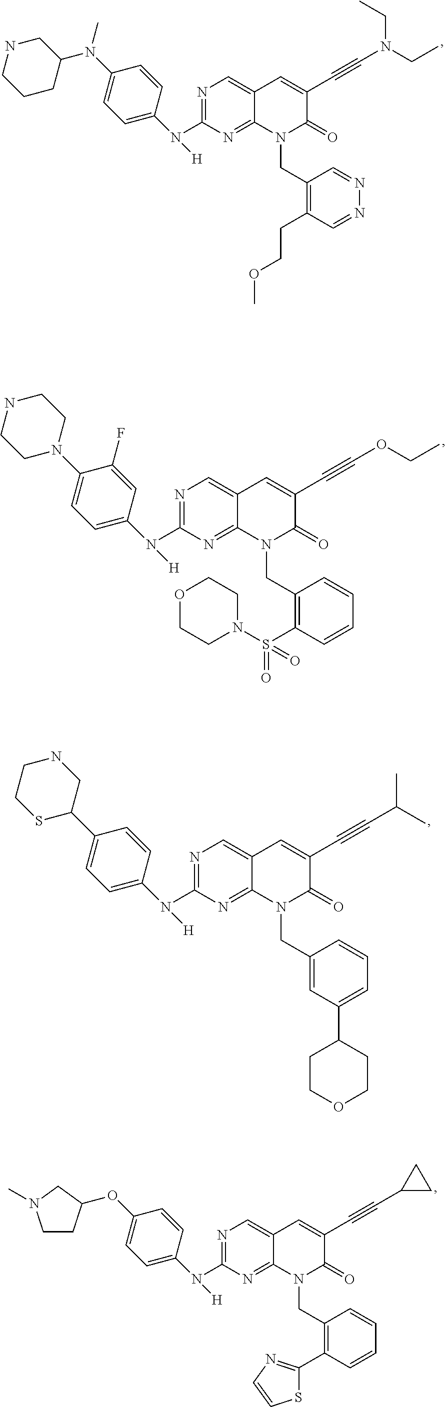

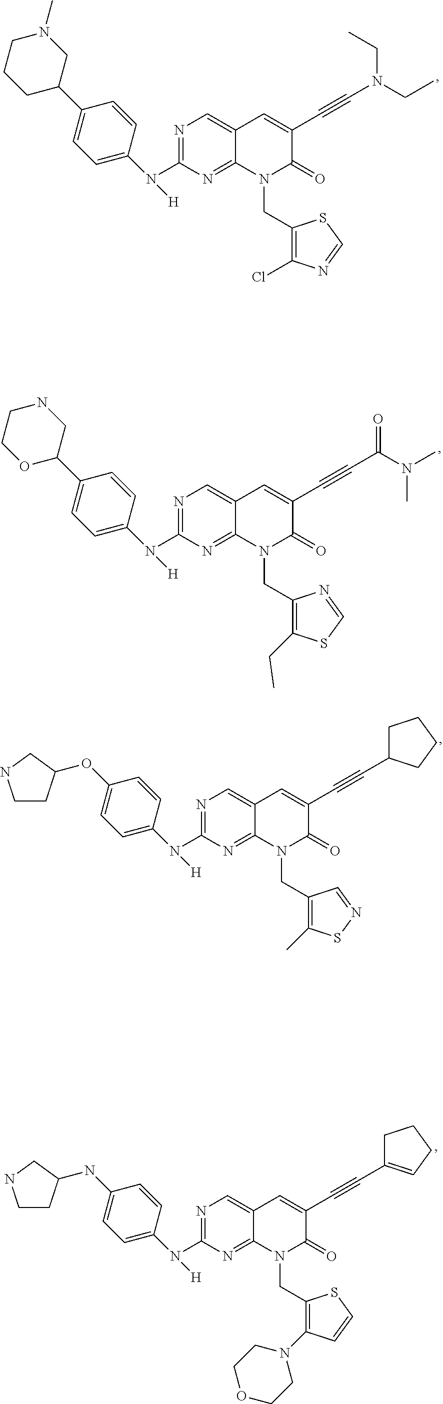 [SMILES: CCN(C#Cc1cc2cnc(Nc3ccc(C4CCCN(C)C4)cc3)nc2n(Cc2scnc2Cl)c1=O)CC ; CCc1scnc1Cn1c(=O)c(C#CC(=O)N(C)C)cc2cnc(Nc3ccc(C4CNCCO4)cc3)nc21 ; Cc1sncc1Cn1c(=O)c(C#CC2CCCC2)cc2cnc(Nc3ccc(OC4CCNC4)cc3)nc21 ; O=c1c(C#CC2=CCCC2)cc2cnc(Nc3ccc(NC4CCNC4)cc3)nc2n1Cc1sccc1N1CCOCC1]

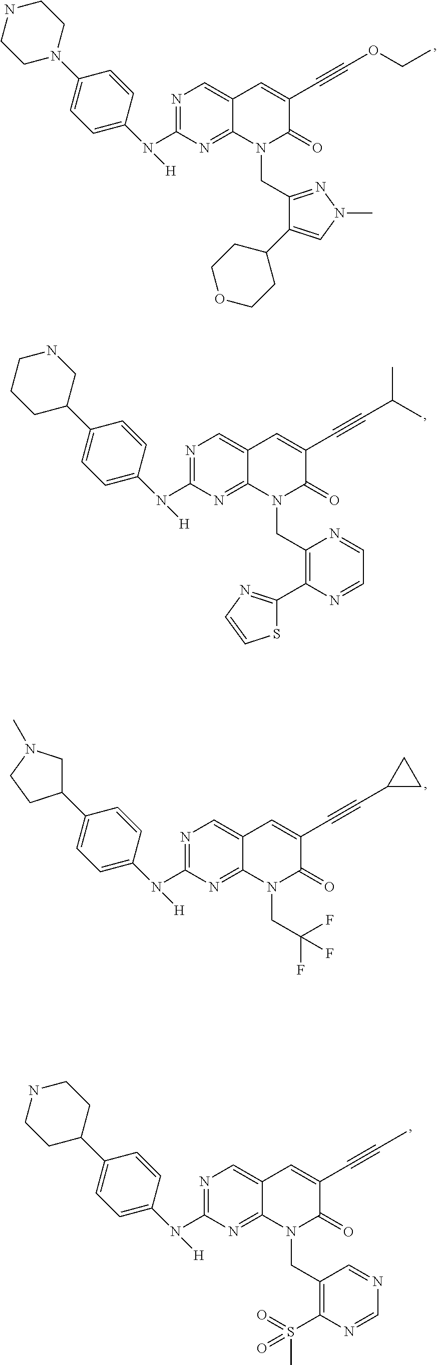 [SMILES: CCOC#Cc1cc2cnc(Nc3ccc(N4CCNCC4)cc3)nc2n(Cc2nn(C)cc2C2CCOCC2)c1=O ; CC(C)C#Cc1cc2cnc(Nc3ccc(C4CCCNC4)cc3)nc2n(Cc2nccnc2-c2nccs2)c1=O ; CN1CCC(c2ccc(Nc3ncc4cc(C#CC5CC5)c(=O)n(CC(F)(F)F)c4n3)cc2)C1 ; CC#Cc1cc2cnc(Nc3ccc(C4CCNCC4)cc3)nc2n(Cc2cncnc2S(C)(=O)=O)c1=O]

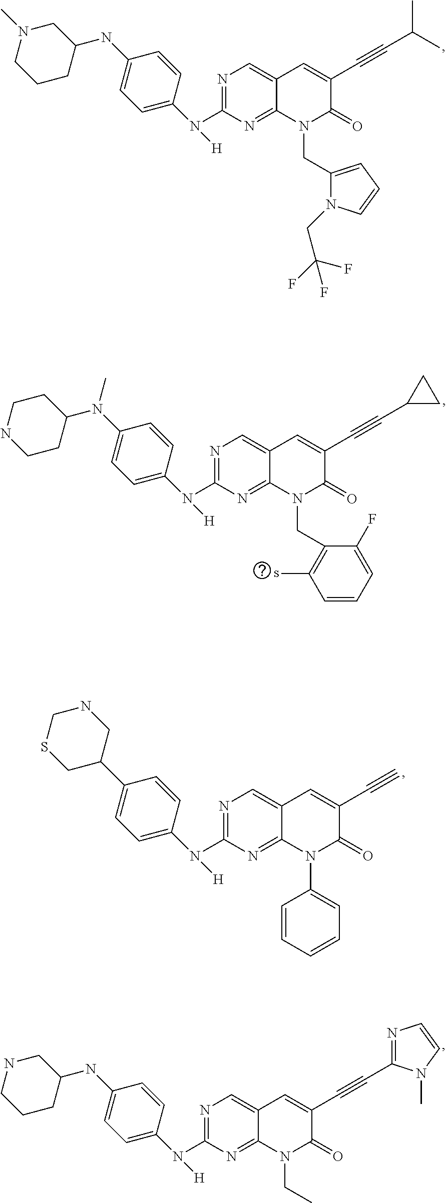

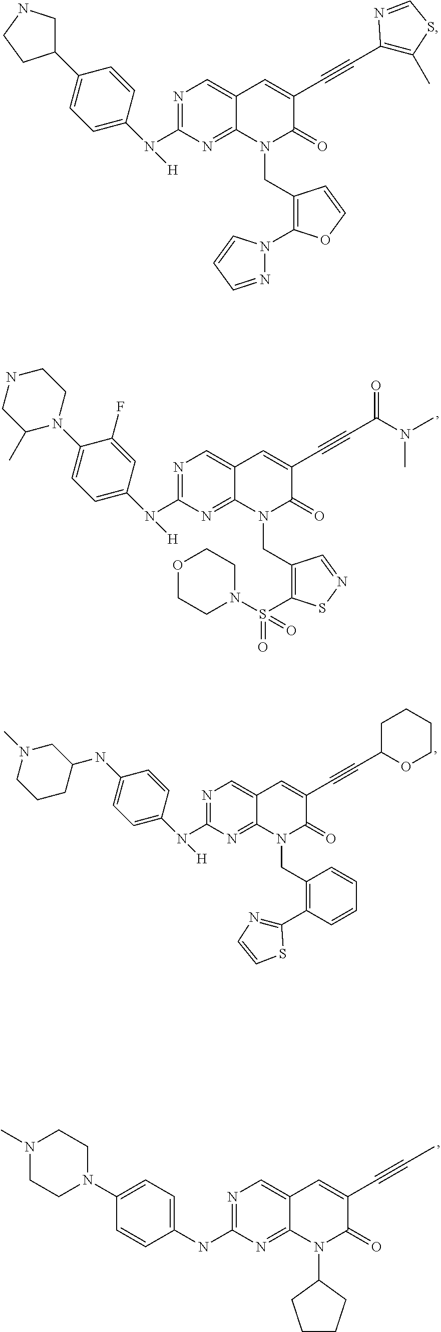

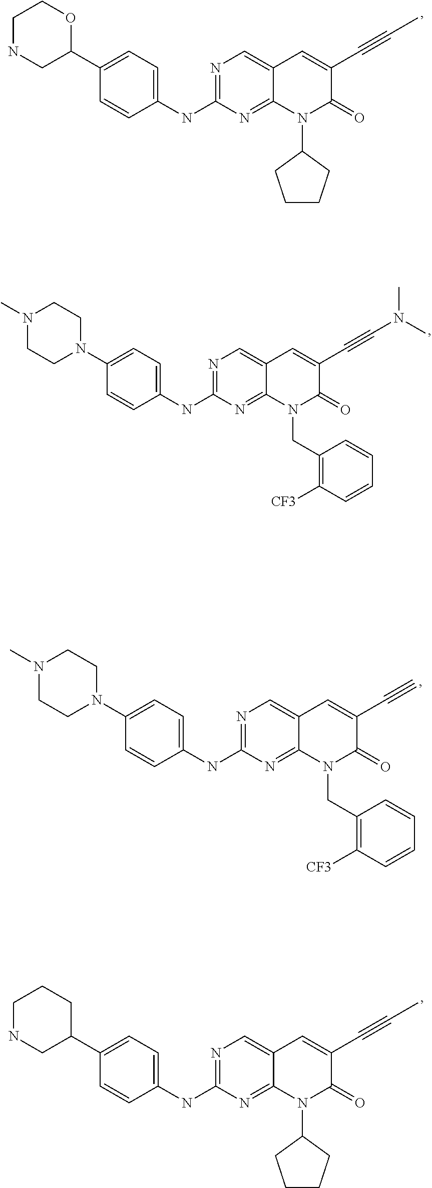

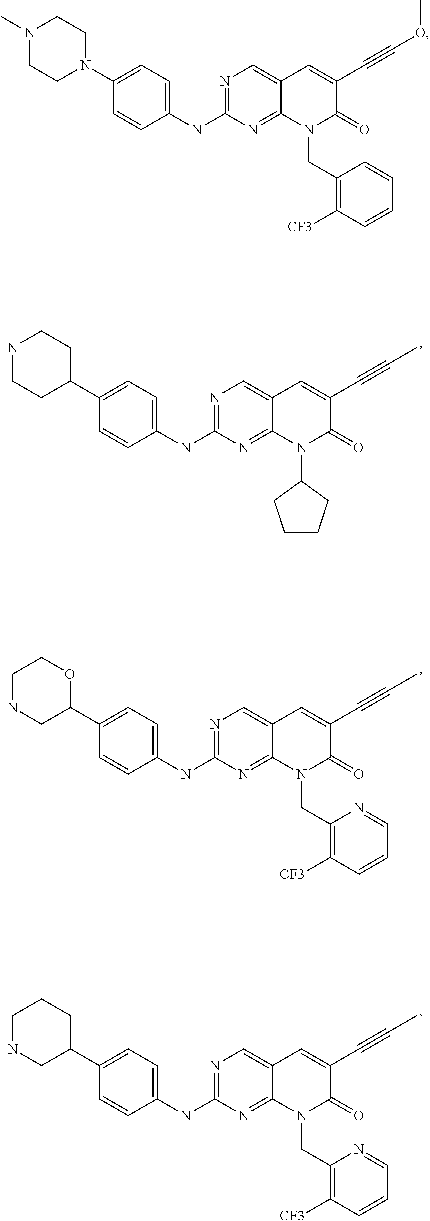

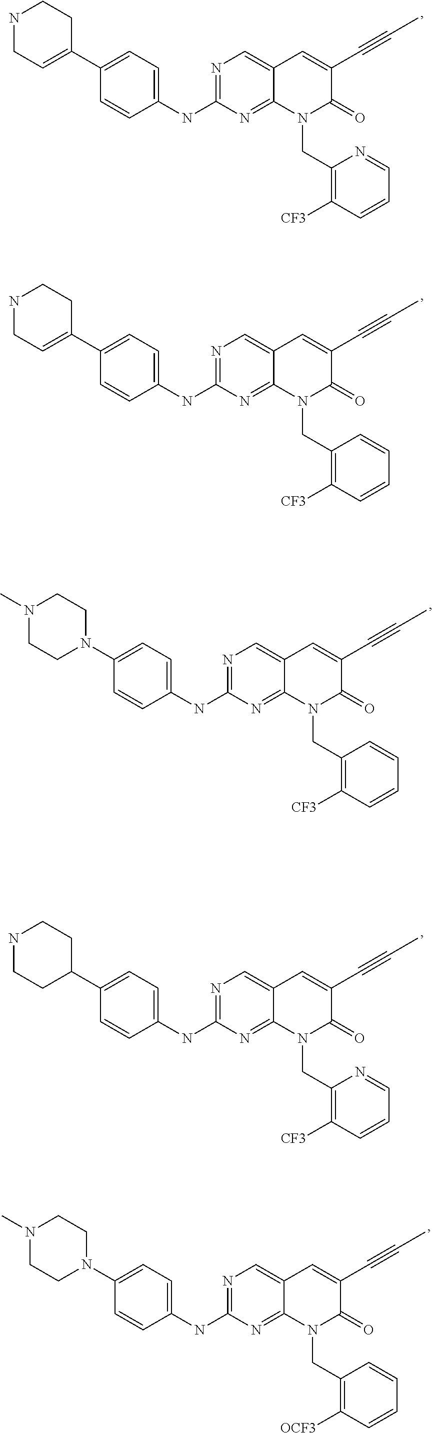

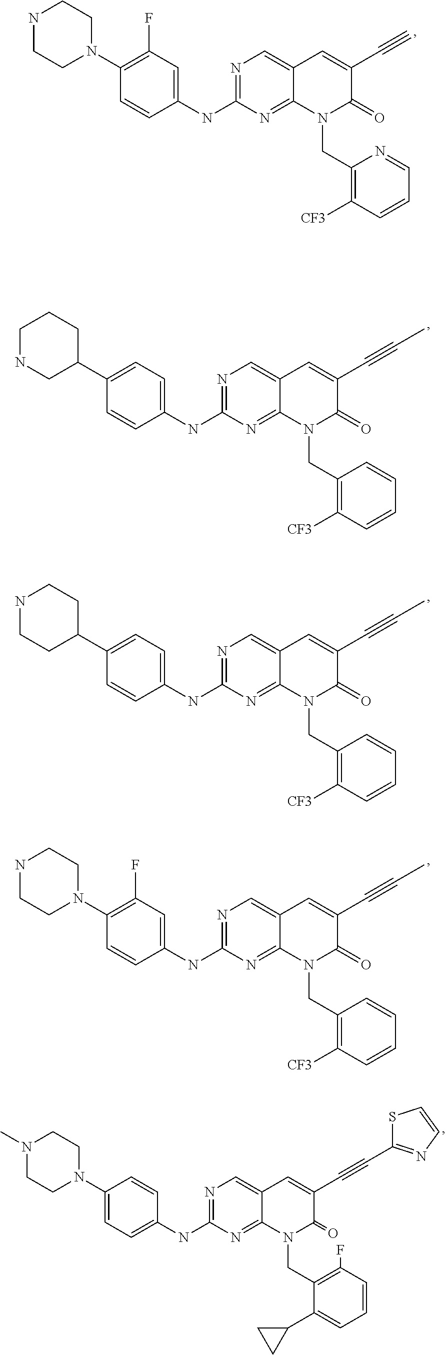

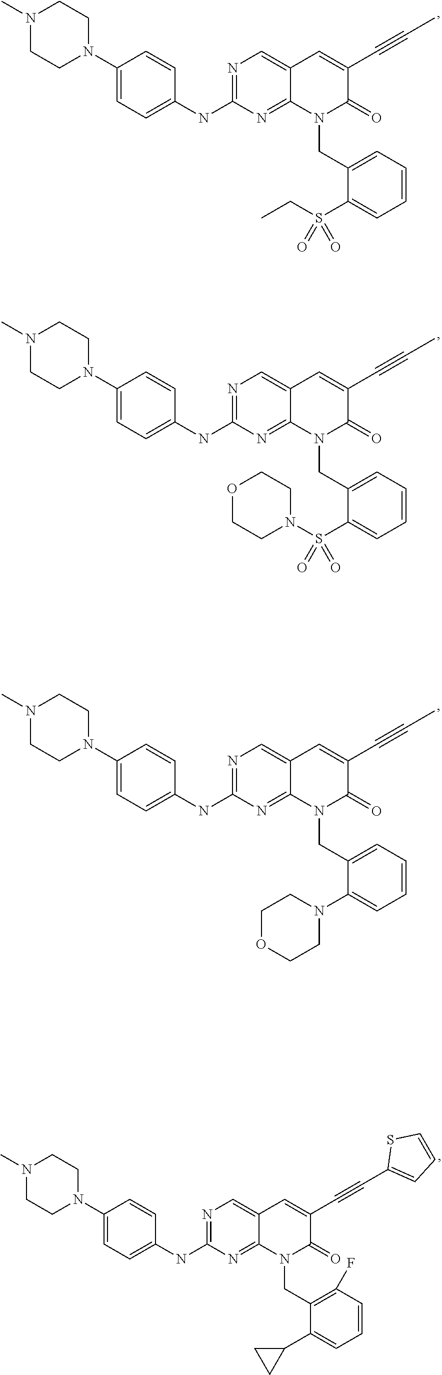

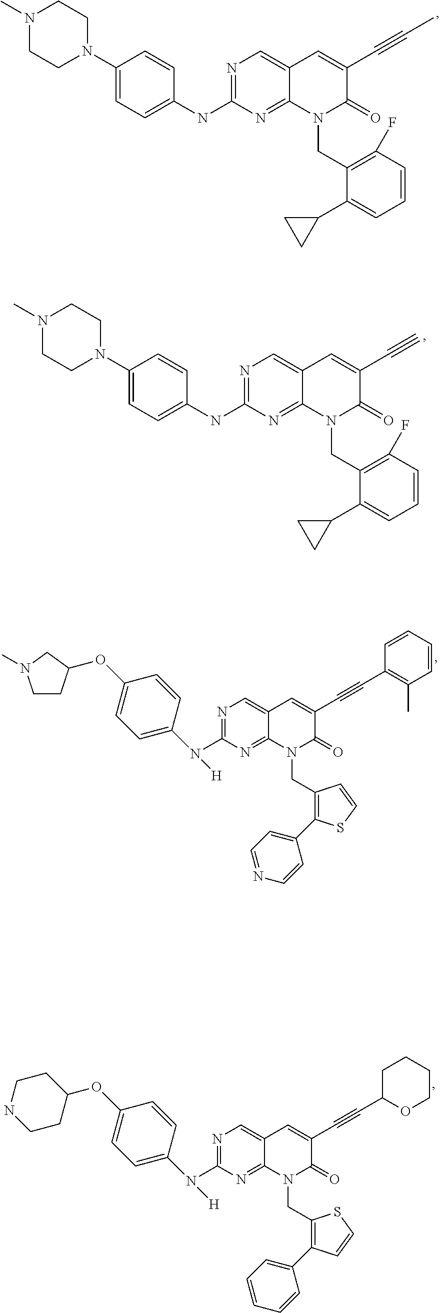

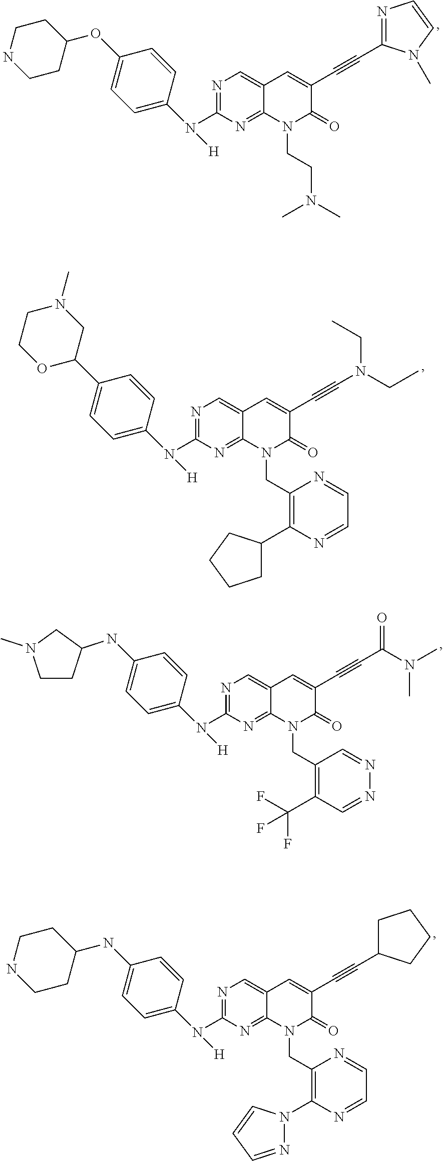

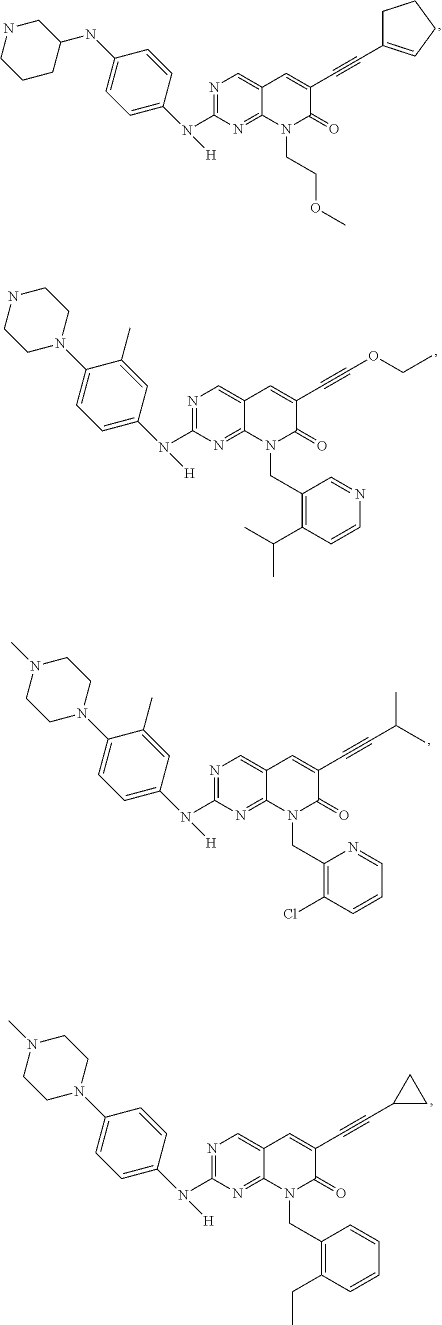

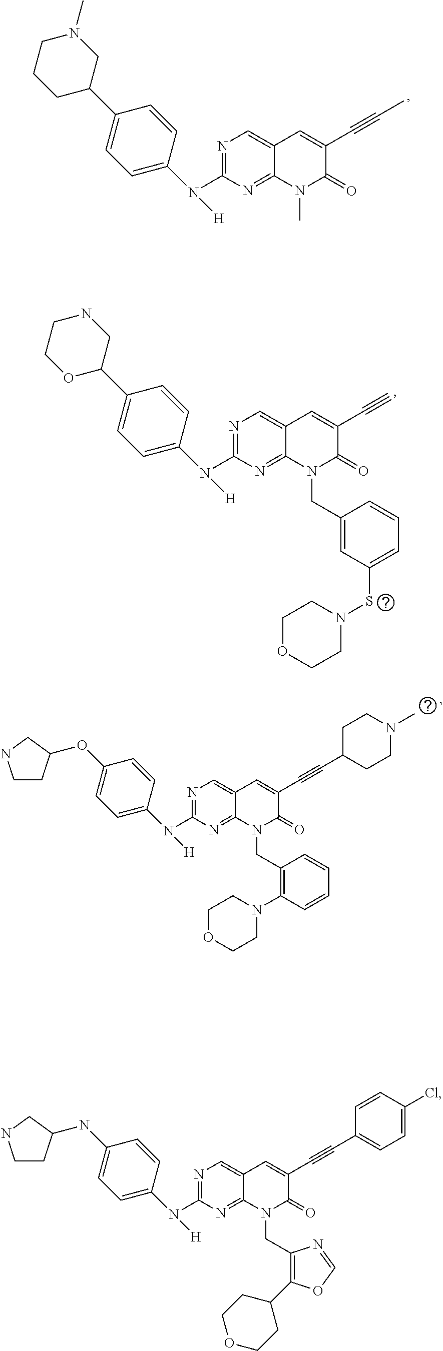 [SMILES: CC#Cc1cc2cnc(Nc3ccc(C4CCCN(C)C4)cc3)nc2n(C)c1=O ; C#Cc1cc2cnc(Nc3ccc(C4CNCCO4)cc3)nc2n(Cc2cccc(SN3CCOCC3)c2)c1=O ; CN1CCC(C#Cc2cc3cnc(Nc4ccc(OC5CCNC5)cc4)nc3n(Cc3ccccc3N3CCOCC3)c2=O)CC1 ; O=c1c(C#Cc2ccc(Cl)cc2)cc2cnc(Nc3ccc(NC4CCNC4)cc3)nc2n1Cc1ncoc1C1CCOCC1]

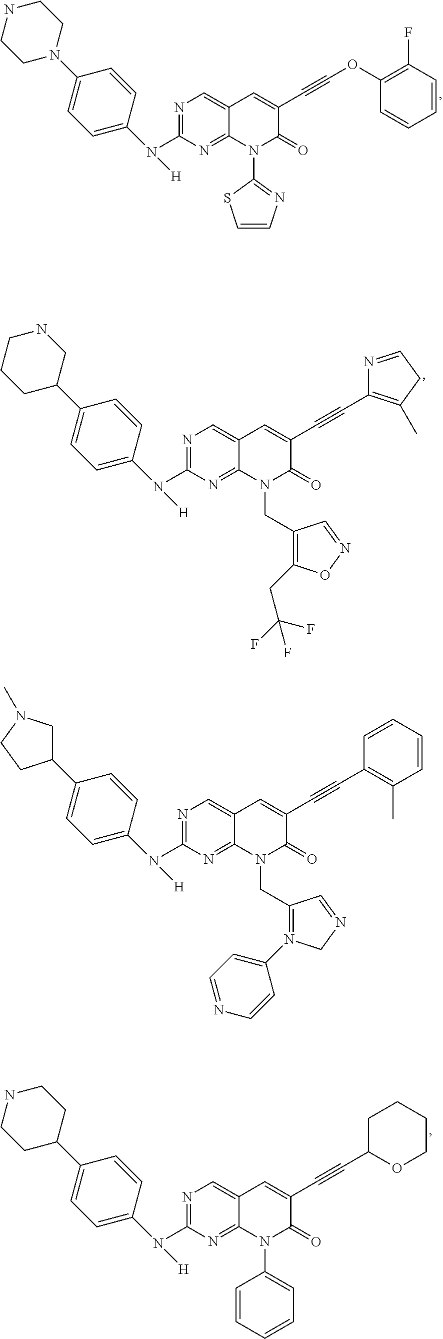

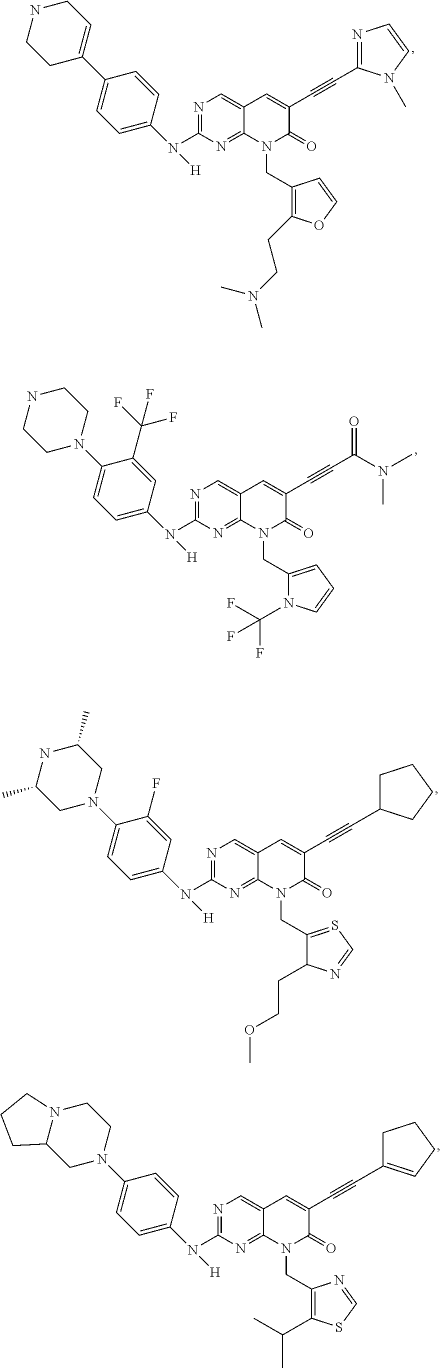

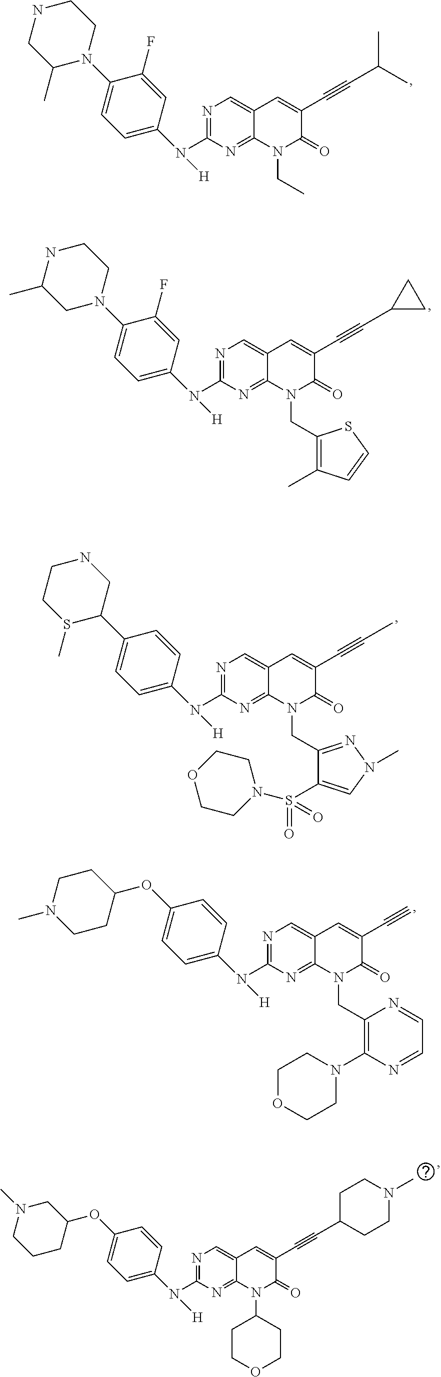

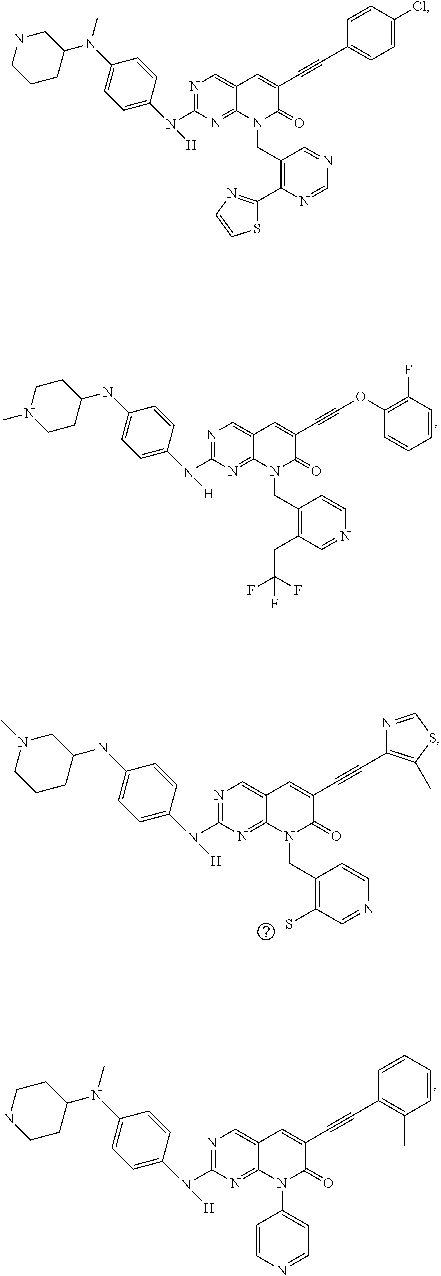 [SMILES: CN(c1ccc(Nc2ncc3cc(C#Cc4ccc(Cl)cc4)c(=O)n(Cc4cncnc4-c4nccs4)c3n2)cc1)C1CCCNC1 ; CN1CCC(Nc2ccc(Nc3ncc4cc(C#COc5ccccc5F)c(=O)n(Cc5ccncc5CC(F)(F)F)c4n3)cc2)CC1 ; Cc1scnc1C#Cc1cc2cnc(Nc3ccc(NC4CCCN(C)C4)cc3)nc2n(Cc2ccncc2S)c1=O ; Cc1ccccc1C#Cc1cc2cnc(Nc3ccc(N(C)C4CCNCC4)cc3)nc2n(-c2ccncc2)c1=O]

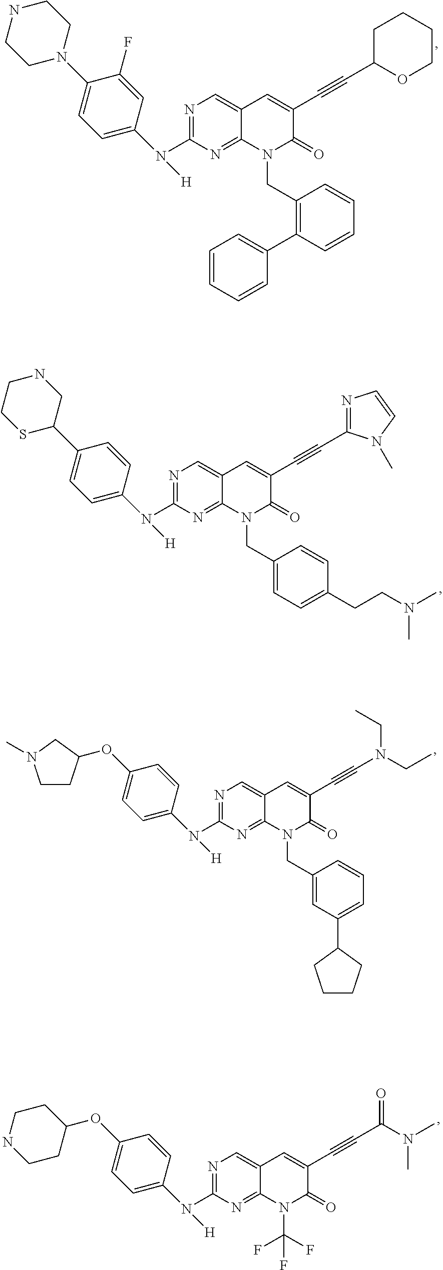

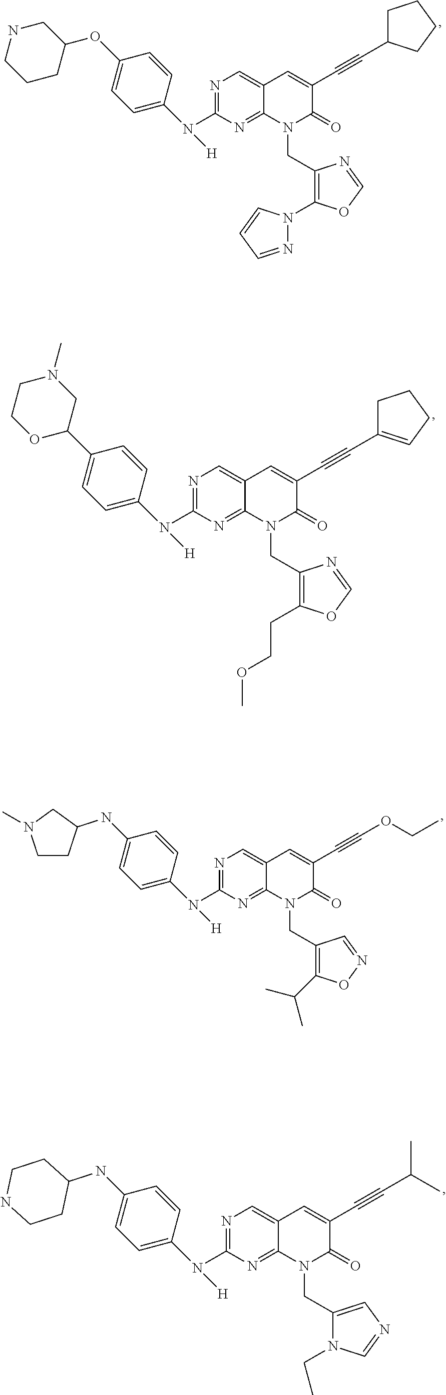 [SMILES: O=c1c(C#CC2CCCC2)cc2cnc(Nc3ccc(OC4CCCNC4)cc3)nc2n1Cc1ncoc1-n1cccn1 ; COCCc1ocnc1Cn1c(=O)c(C#CC2=CCCC2)cc2cnc(Nc3ccc(C4CN(C)CCO4)cc3)nc21 ; CCOC#Cc1cc2cnc(Nc3ccc(NC4CCN(C)C4)cc3)nc2n(Cc2cnoc2C(C)C)c1=O ; CCn1cncc1Cn1c(=O)c(C#CC(C)C)cc2cnc(Nc3ccc(NC4CCNCC4)cc3)nc21]

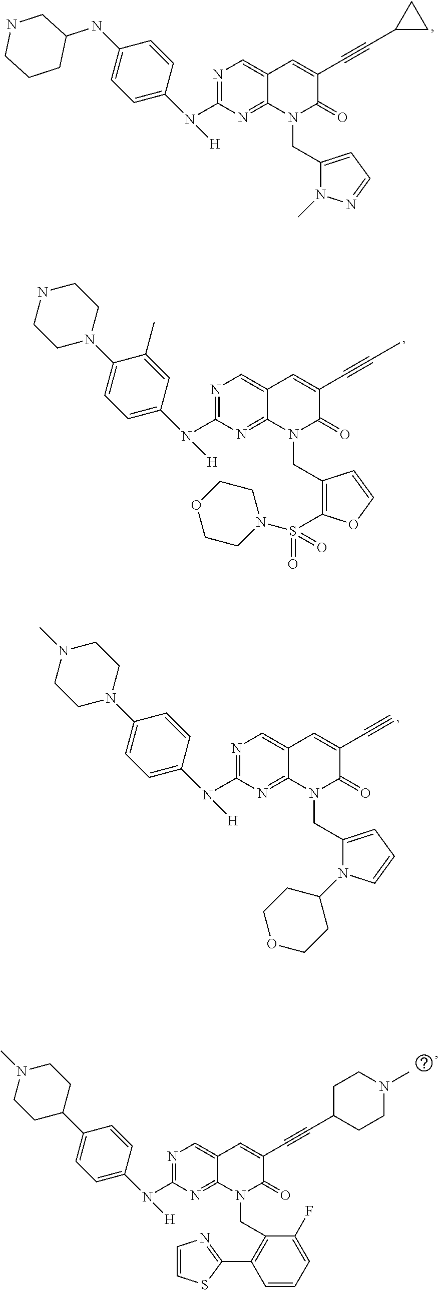

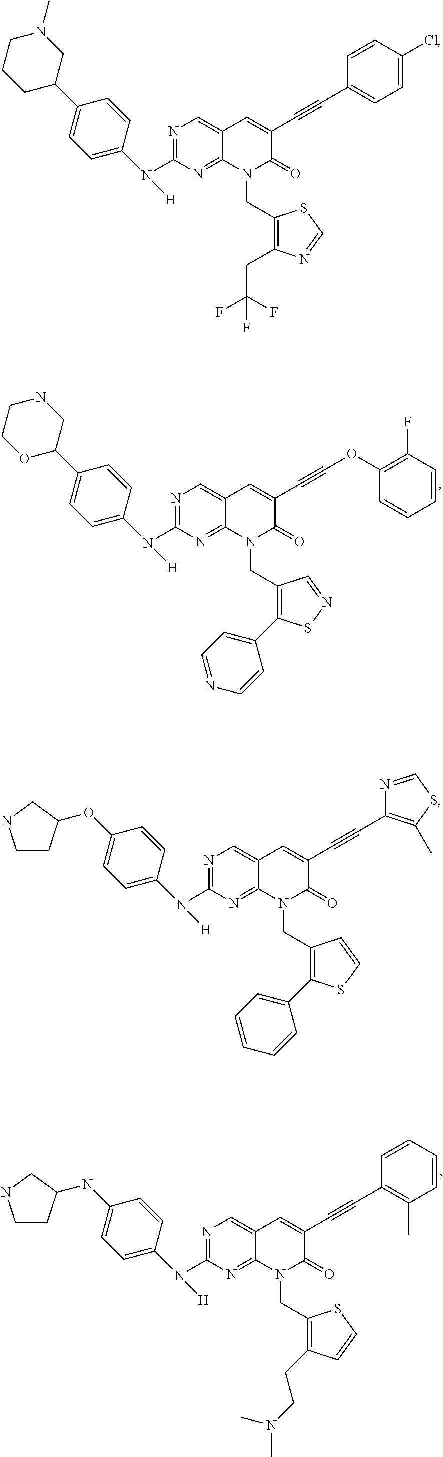 [SMILES: CN1CCCC(c2ccc(Nc3ncc4cc(C#Cc5ccc(Cl)cc5)c(=O)n(Cc5scnc5CC(F)(F)F)c4n3)cc2)C1 ; O=c1c(C#COc2ccccc2F)cc2cnc(Nc3ccc(C4CNCCO4)cc3)nc2n1Cc1cnsc1-c1ccncc1 ; Cc1scnc1C#Cc1cc2cnc(Nc3ccc(OC4CCNC4)cc3)nc2n(Cc2ccsc2-c2ccccc2)c1=O ; Cc1ccccc1C#Cc1cc2cnc(Nc3ccc(NC4CCNC4)cc3)nc2n(Cc2sccc2CCN(C)C)c1=O]

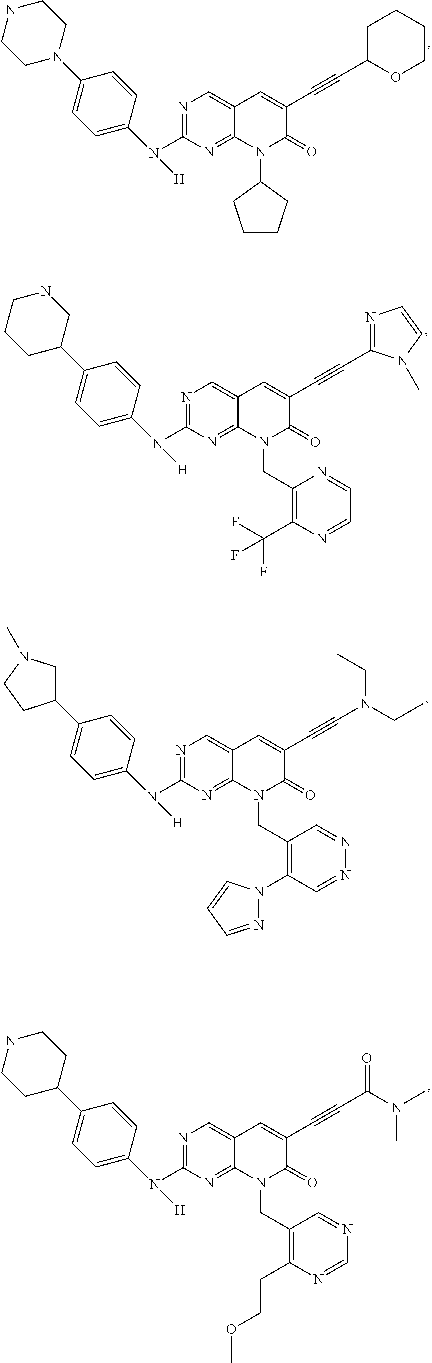 [SMILES: O=c1c(C#CC2CCCCO2)cc2cnc(Nc3ccc(N4CCNCC4)cc3)nc2n1C1CCCC1 ; Cn1ccnc1C#Cc1cc2cnc(Nc3ccc(C4CCCNC4)cc3)nc2n(Cc2nccnc2C(F)(F)F)c1=O ; CCN(C#Cc1cc2cnc(Nc3ccc(C4CCN(C)C4)cc3)nc2n(Cc2cnncc2-n2cccn2)c1=O)CC ; COCCc1ncncc1Cn1c(=O)c(C#CC(=O)N(C)C)cc2cnc(Nc3ccc(C4CCNCC4)cc3)nc21]

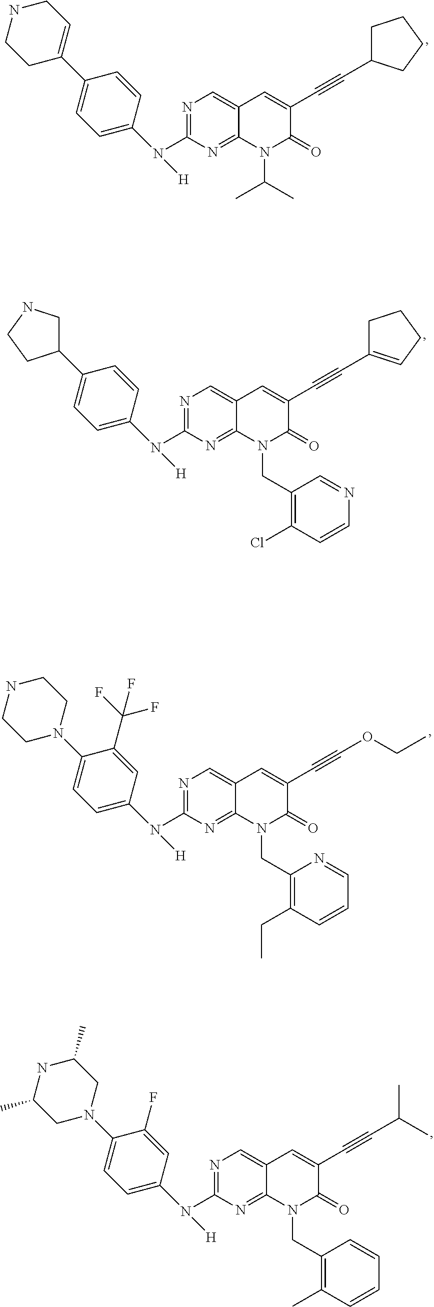

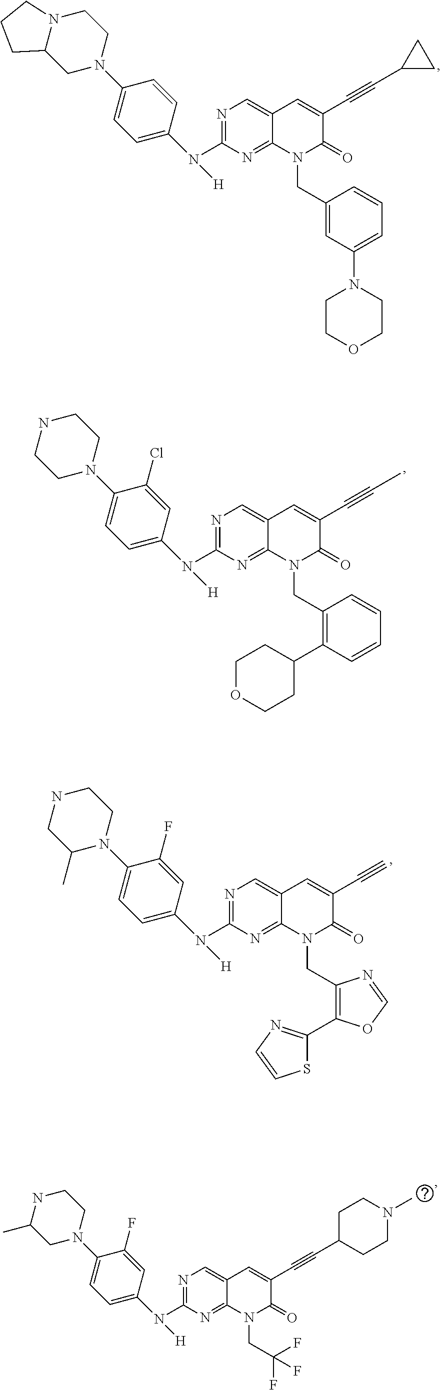

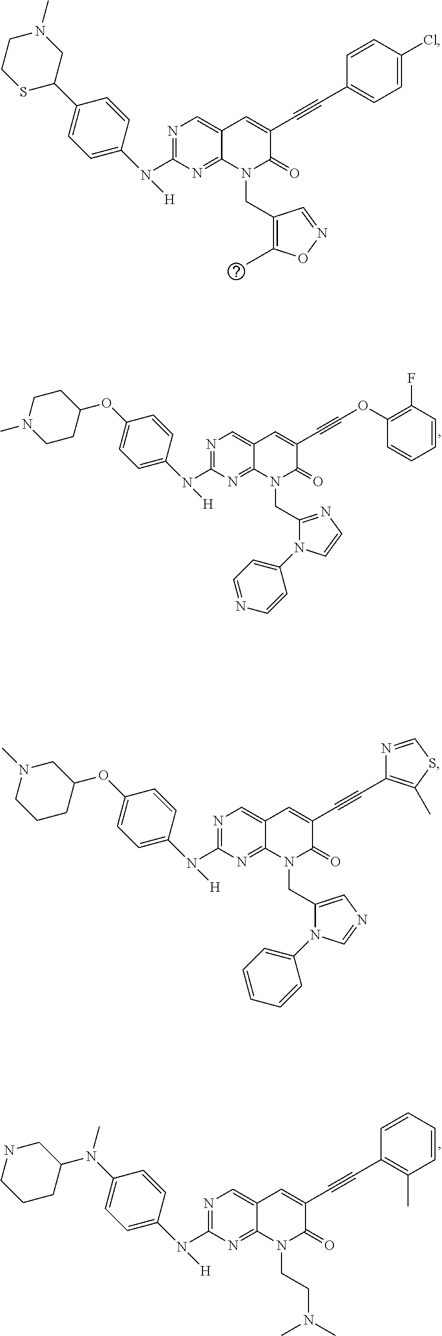 [SMILES: Cc1oncc1Cn1c(=O)c(C#Cc2ccc(Cl)cc2)cc2cnc(Nc3ccc(C4CN(C)CCS4)cc3)nc21 ; CN1CCC(Oc2ccc(Nc3ncc4cc(C#COc5ccccc5F)c(=O)n(Cc5nccn5-c5ccncc5)c4n3)cc2)CC1 ; Cc1scnc1C#Cc1cc2cnc(Nc3ccc(OC4CCCN(C)C4)cc3)nc2n(Cc2cncn2-c2ccccc2)c1=O ; Cc1ccccc1C#Cc1cc2cnc(Nc3ccc(N(C)C4CCCNC4)cc3)nc2n(CCN(C)C)c1=O]

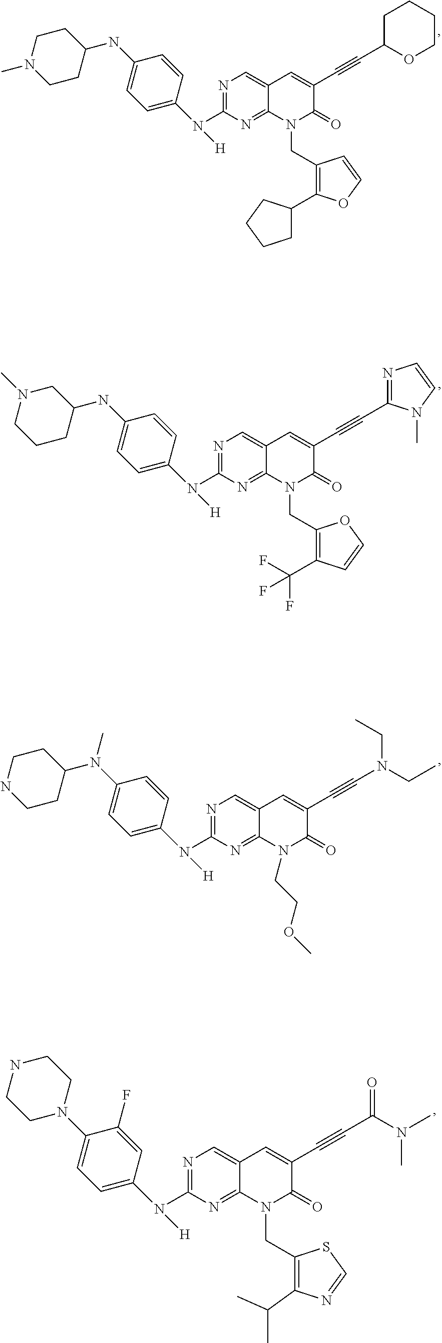 [SMILES: CN1CCC(Nc2ccc(Nc3ncc4cc(C#CC5CCCCO5)c(=O)n(Cc5ccoc5C5CCCC5)c4n3)cc2)CC1 ; CN1CCCC(Nc2ccc(Nc3ncc4cc(C#Cc5nccn5C)c(=O)n(Cc5occc5C(F)(F)F)c4n3)cc2)C1 ; CCN(C#Cc1cc2cnc(Nc3ccc(N(C)C4CCNCC4)cc3)nc2n(CCOC)c1=O)CC ; CC(C)c1ncsc1Cn1c(=O)c(C#CC(=O)N(C)C)cc2cnc(Nc3ccc(N4CCNCC4)c(F)c3)nc21]

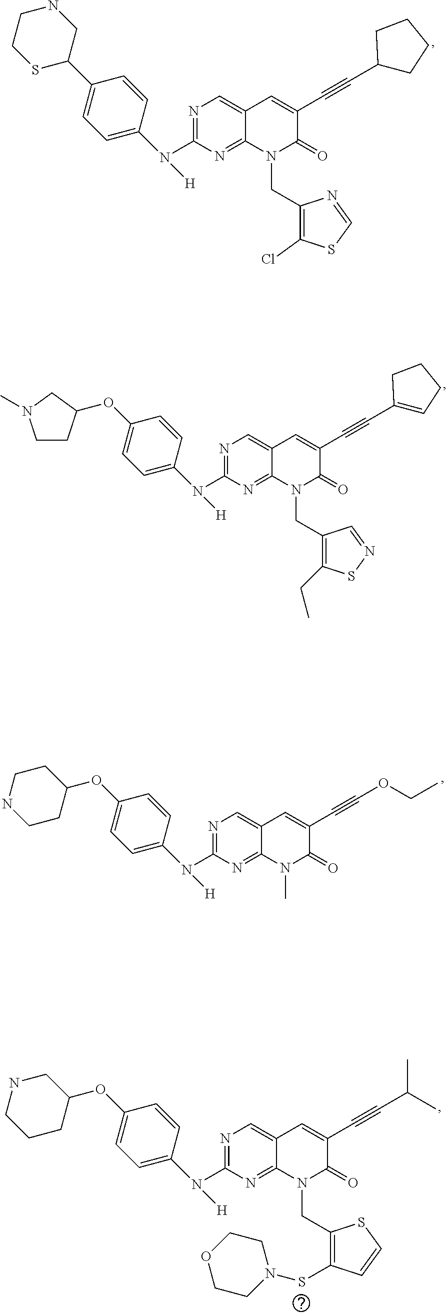 [SMILES: O=c1c(C#CC2CCCC2)cc2cnc(Nc3ccc(C4CNCCS4)cc3)nc2n1Cc1ncsc1Cl ; CCc1sncc1Cn1c(=O)c(C#CC2=CCCC2)cc2cnc(Nc3ccc(OC4CCN(C)C4)cc3)nc21 ; CCOC#Cc1cc2cnc(Nc3ccc(OC4CCNCC4)cc3)nc2n(C)c1=O ; CC(C)C#Cc1cc2cnc(Nc3ccc(OC4CCCNC4)cc3)nc2n(Cc2sccc2SN2CCOCC2)c1=O]

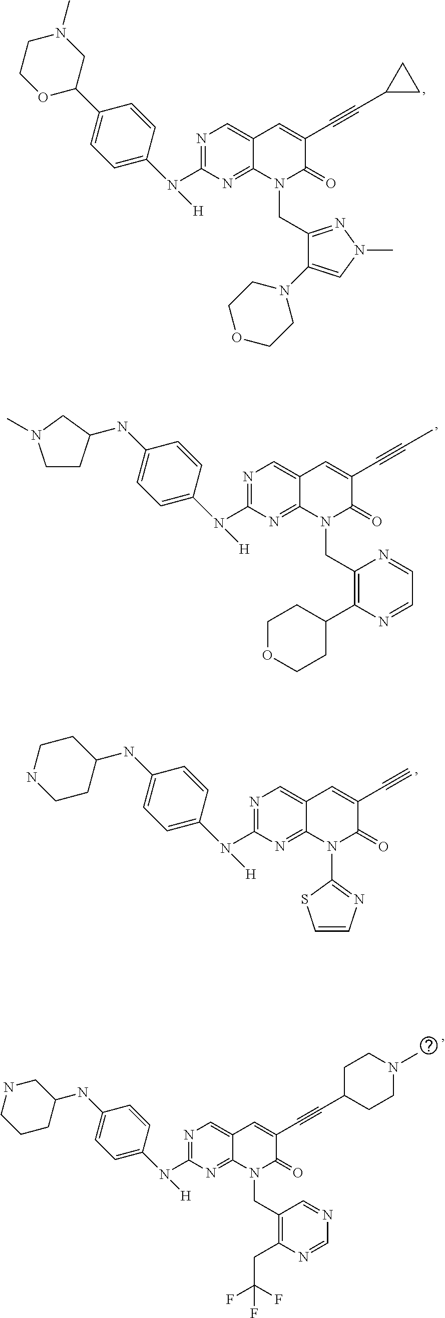 [SMILES: CN1CCOC(c2ccc(Nc3ncc4cc(C#CC5CC5)c(=O)n(Cc5nn(C)cc5N5CCOCC5)c4n3)cc2)C1 ; CC#Cc1cc2cnc(Nc3ccc(NC4CCN(C)C4)cc3)nc2n(Cc2nccnc2C2CCOCC2)c1=O ; C#Cc1cc2cnc(Nc3ccc(NC4CCNCC4)cc3)nc2n(-c2nccs2)c1=O ; CN1CCC(C#Cc2cc3cnc(Nc4ccc(NC5CCCNC5)cc4)nc3n(Cc3cncnc3CC(F)(F)F)c2=O)CC1]

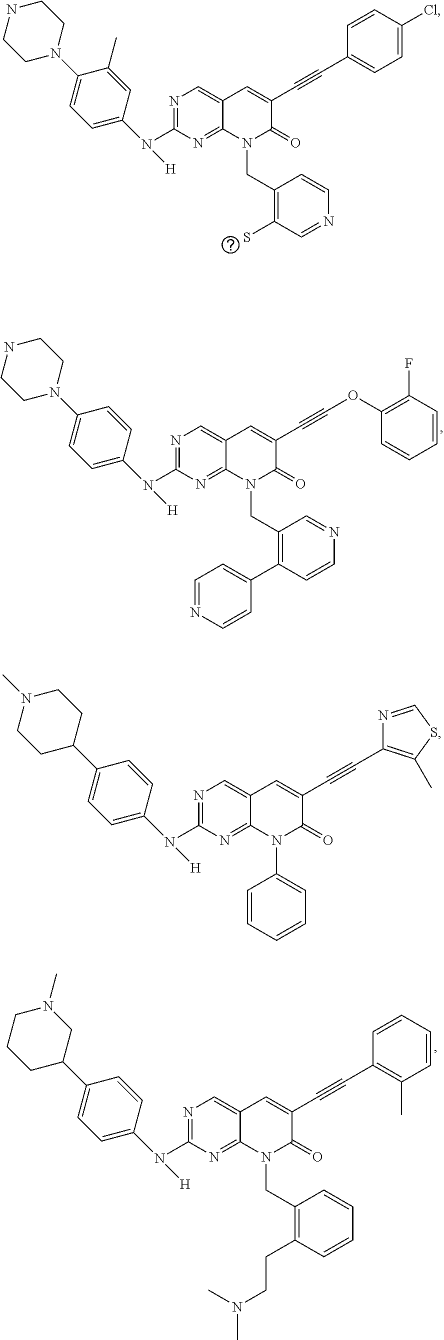 [SMILES: Cc1cc(Nc2ncc3cc(C#Cc4ccc(Cl)cc4)c(=O)n(Cc4ccncc4S)c3n2)ccc1N1CCNCC1 ; O=c1c(C#COc2ccccc2F)cc2cnc(Nc3ccc(N4CCNCC4)cc3)nc2n1Cc1cnccc1-c1ccncc1 ; Cc1scnc1C#Cc1cc2cnc(Nc3ccc(C4CCN(C)CC4)cc3)nc2n(-c2ccccc2)c1=O ; Cc1ccccc1C#Cc1cc2cnc(Nc3ccc(C4CCCN(C)C4)cc3)nc2n(Cc2ccccc2CCN(C)C)c1=O]

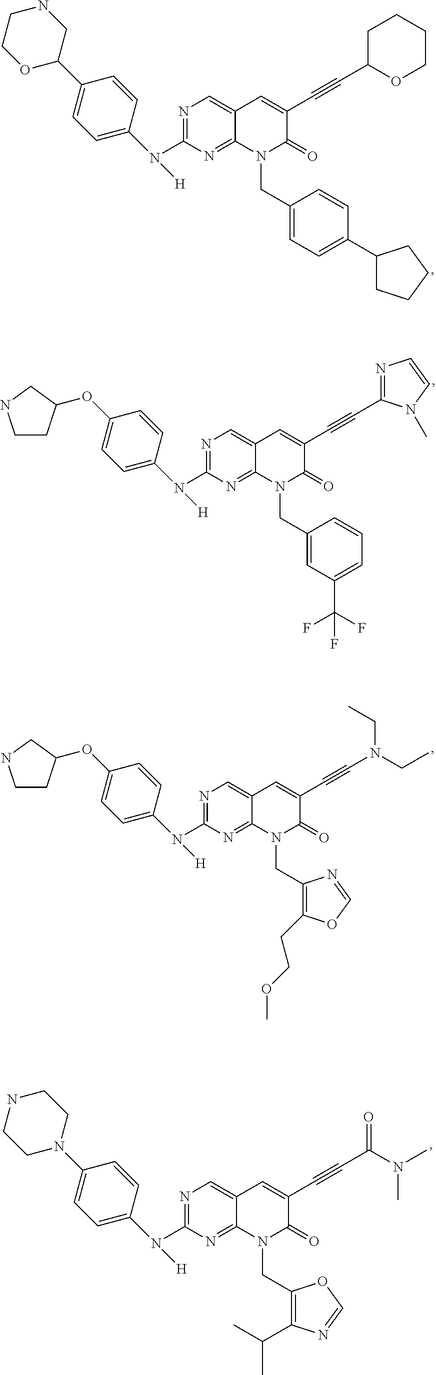 [SMILES: O=c1c(C#CC2CCCCO2)cc2cnc(Nc3ccc(C4CNCCO4)cc3)nc2n1Cc1ccc(C2CCCC2)cc1 ; Cn1ccnc1C#Cc1cc2cnc(Nc3ccc(OC4CCNC4)cc3)nc2n(Cc2cccc(C(F)(F)F)c2)c1=O ; CCN(C#Cc1cc2cnc(Nc3ccc(OC4CCNC4)cc3)nc2n(Cc2ncoc2CCOC)c1=O)CC ; CC(C)c1ncoc1Cn1c(=O)c(C#CC(=O)N(C)C)cc2cnc(Nc3ccc(N4CCNCC4)cc3)nc21]

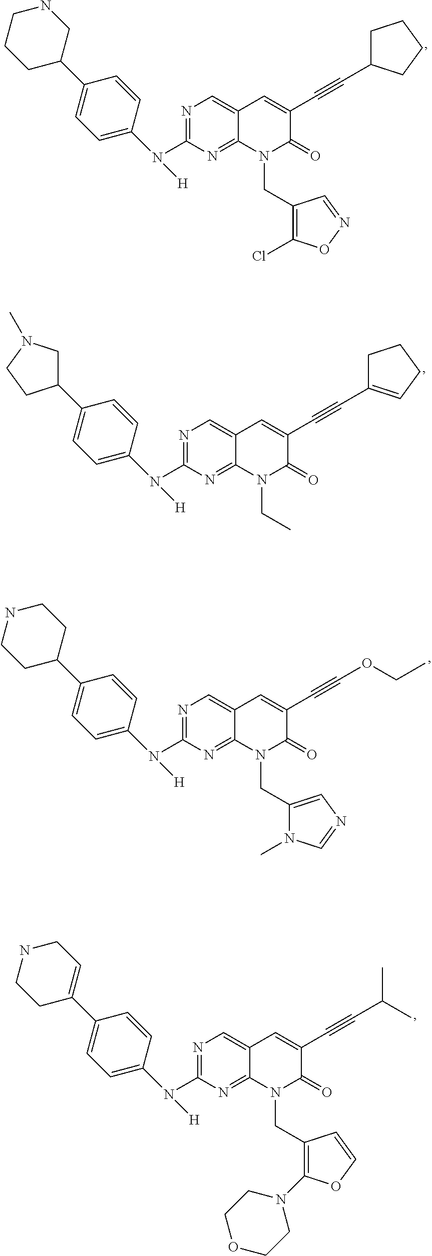 [SMILES: O=c1c(C#CC2CCCC2)cc2cnc(Nc3ccc(C4CCCNC4)cc3)nc2n1Cc1cnoc1Cl ; CCn1c(=O)c(C#CC2=CCCC2)cc2cnc(Nc3ccc(C4CCN(C)C4)cc3)nc21 ; CCOC#Cc1cc2cnc(Nc3ccc(C4CCNCC4)cc3)nc2n(Cc2cncn2C)c1=O ; CC(C)C#Cc1cc2cnc(Nc3ccc(C4=CCNCC4)cc3)nc2n(Cc2ccoc2N2CCOCC2)c1=O]

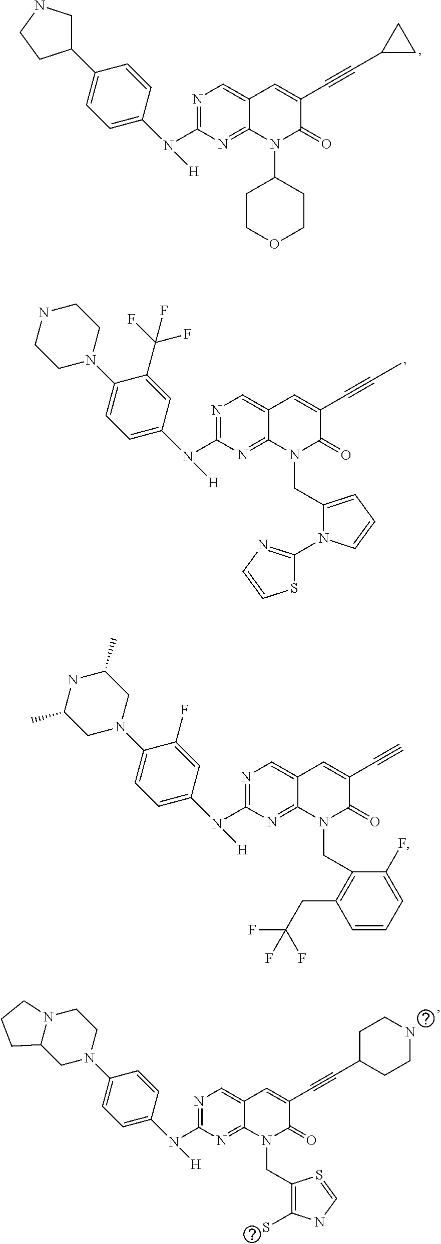

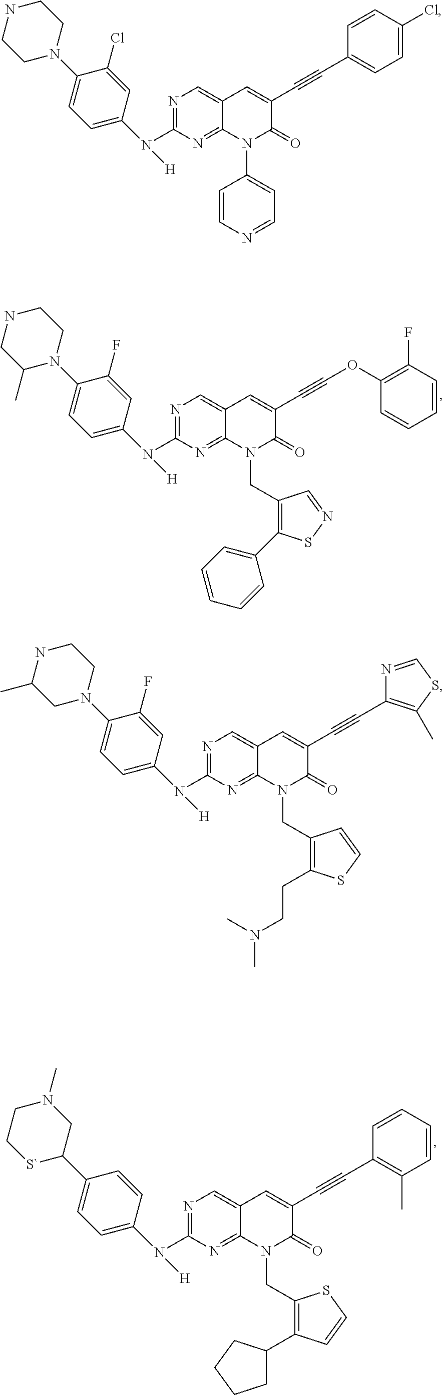

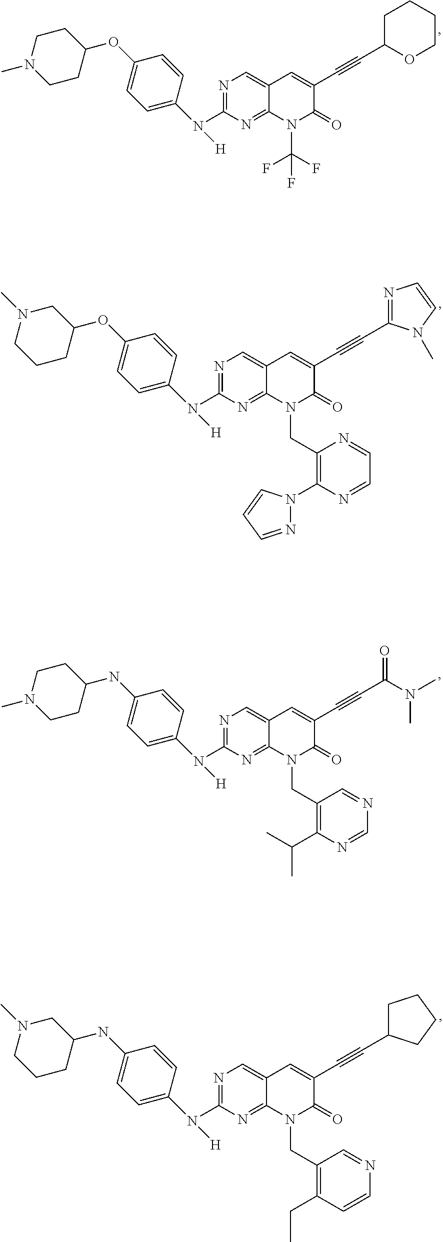

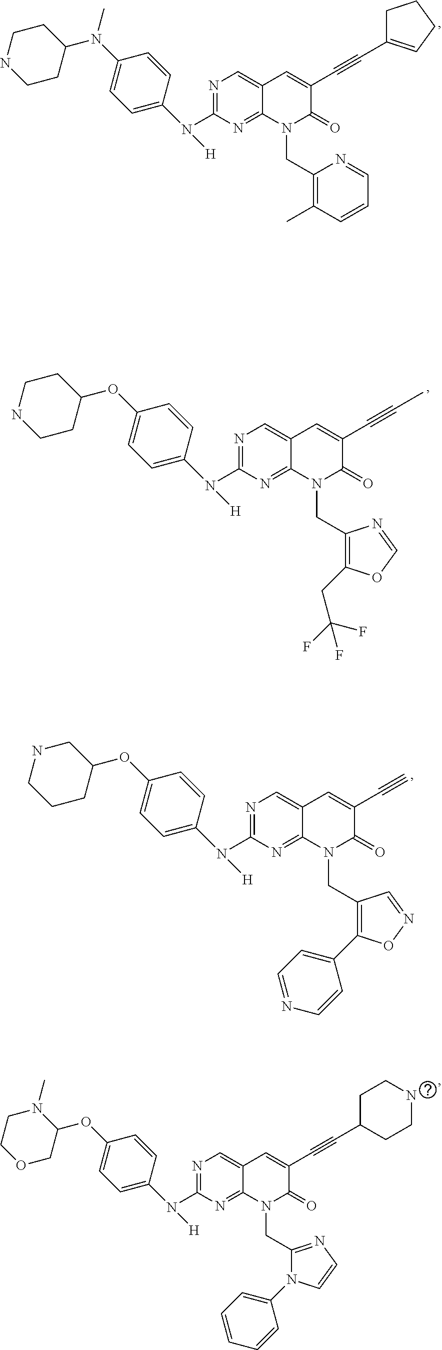 [SMILES: Cc1cccnc1Cn1c(=O)c(C#CC2=CCCC2)cc2cnc(Nc3ccc(N(C)C4CCNCC4)cc3)nc21 ; CC#Cc1cc2cnc(Nc3ccc(OC4CCNCC4)cc3)nc2n(Cc2ncoc2CC(F)(F)F)c1=O ; C#Cc1cc2cnc(Nc3ccc(OC4CCCNC4)cc3)nc2n(Cc2cnoc2-c2ccncc2)c1=O ; CN1CCOCC1Oc1ccc(Nc2ncc3cc(C#CC4CCNCC4)c(=O)n(Cc4nccn4-c4ccccc4)c3n2)cc1]

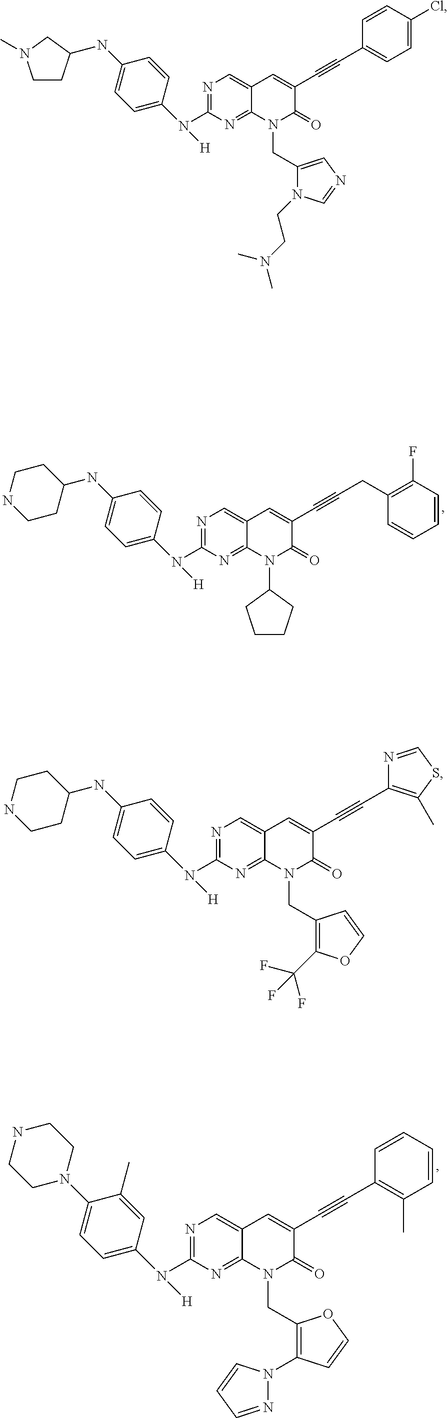 [SMILES: CN(C)CCn1cncc1Cn1c(=O)c(C#Cc2ccc(Cl)cc2)cc2cnc(Nc3ccc(NC4CCN(C)C4)cc3)nc21 ; O=c1c(C#CCc2ccccc2F)cc2cnc(Nc3ccc(NC4CCNCC4)cc3)nc2n1C1CCCC1 ; Cc1scnc1C#Cc1cc2cnc(Nc3ccc(NC4CCNCC4)cc3)nc2n(Cc2ccoc2C(F)(F)F)c1=O ; Cc1ccccc1C#Cc1cc2cnc(Nc3ccc(N4CCNCC4)c(C)c3)nc2n(Cc2occc2-n2cccn2)c1=O]

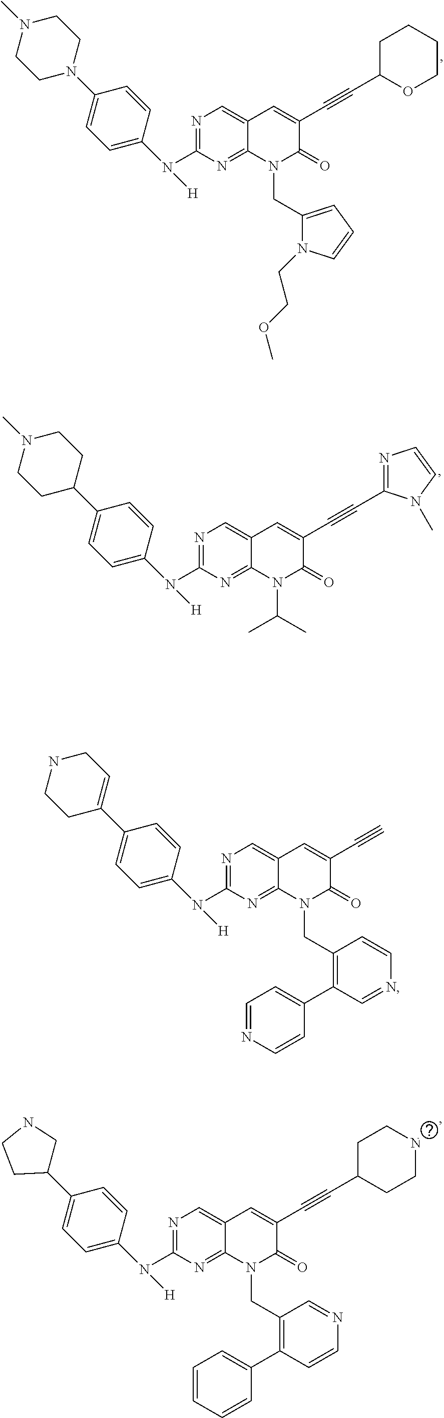 [SMILES: COCCn1cccc1Cn1c(=O)c(C#CC2CCCCO2)cc2cnc(Nc3ccc(N4CCN(C)CC4)cc3)nc21 ; CC(C)n1c(=O)c(C#Cc2nccn2C)cc2cnc(Nc3ccc(C4CCN(C)CC4)cc3)nc21 ; C#Cc1cc2cnc(Nc3ccc(C4=CCNCC4)cc3)nc2n(Cc2ccncc2-c2ccncc2)c1=O ; O=c1c(C#CC2CCNCC2)cc2cnc(Nc3ccc(C4CCNC4)cc3)nc2n1Cc1cnccc1-c1ccccc1]

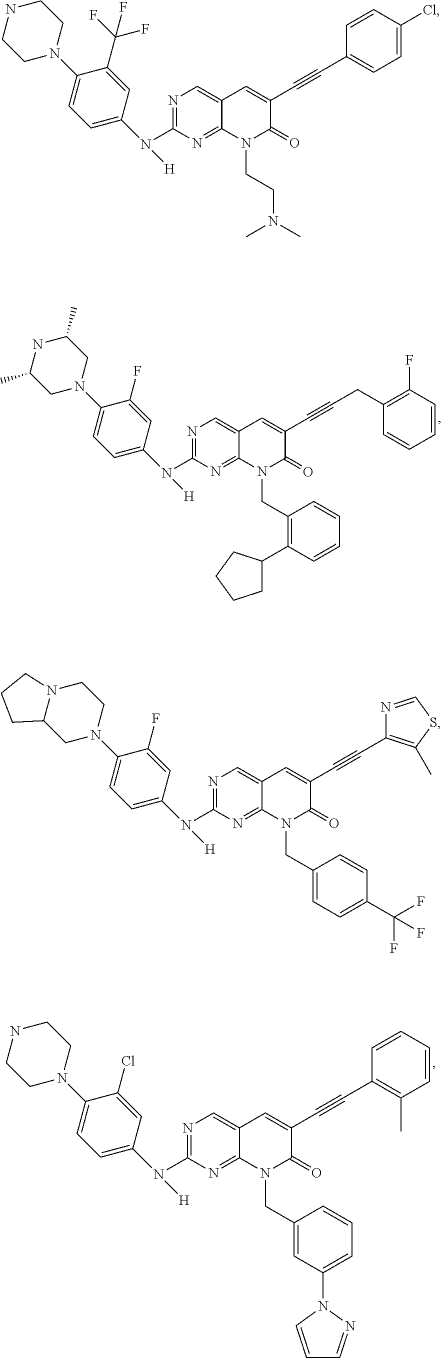 [SMILES: CN(C)CCn1c(=O)c(C#Cc2ccc(Cl)cc2)cc2cnc(Nc3ccc(N4CCNCC4)c(C(F)(F)F)c3)nc21 ; C[C@@H]1CN(c2ccc(Nc3ncc4cc(C#CCc5ccccc5F)c(=O)n(Cc5ccccc5C5CCCC5)c4n3)cc2F)C[C@H](C)N1 ; Cc1scnc1C#Cc1cc2cnc(Nc3ccc(N4CCN5CCCC5C4)c(F)c3)nc2n(Cc2ccc(C(F)(F)F)cc2)c1=O ; Cc1ccccc1C#Cc1cc2cnc(Nc3ccc(N4CCNCC4)c(Cl)c3)nc2n(Cc2cccc(-n3cccn3)c2)c1=O]

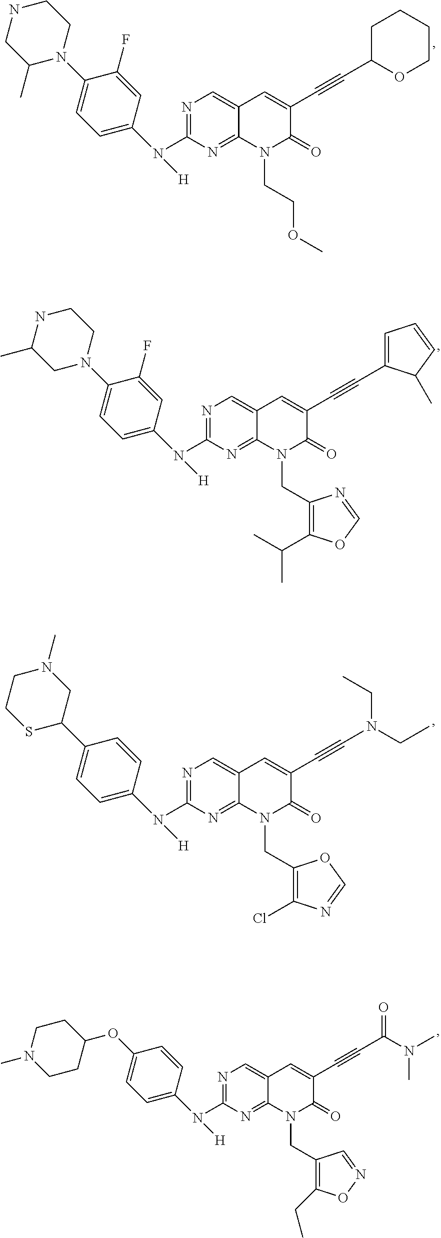

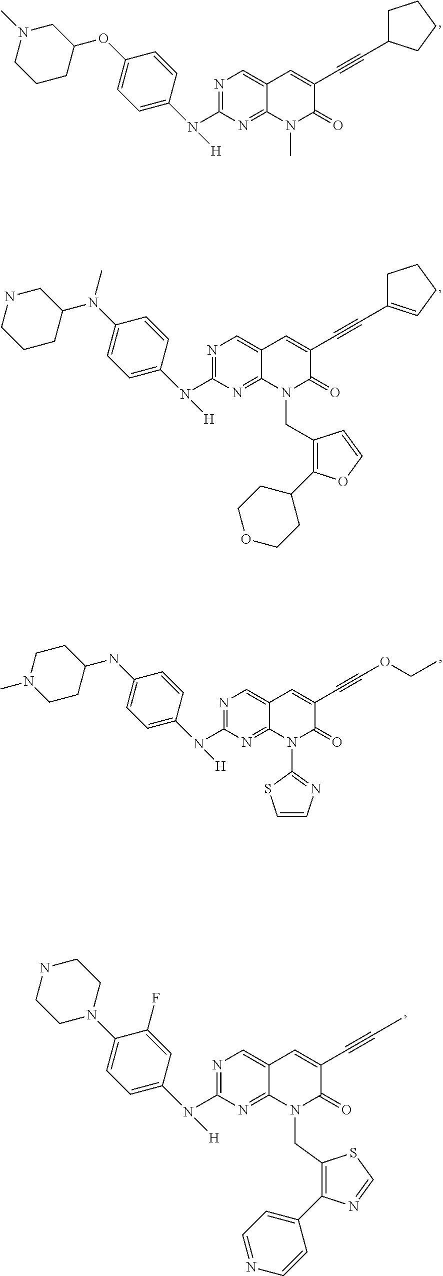 [SMILES: CN1CCCC(Oc2ccc(Nc3ncc4cc(C#CC5CCCC5)c(=O)n(C)c4n3)cc2)C1 ; CN(c1ccc(Nc2ncc3cc(C#CC4=CCCC4)c(=O)n(Cc4ccoc4C4CCOCC4)c3n2)cc1)C1CCCNC1 ; CCOC#Cc1cc2cnc(Nc3ccc(NC4CCN(C)CC4)cc3)nc2n(-c2nccs2)c1=O ; CC#Cc1cc2cnc(Nc3ccc(N4CCNCC4)c(F)c3)nc2n(Cc2scnc2-c2ccncc2)c1=O]

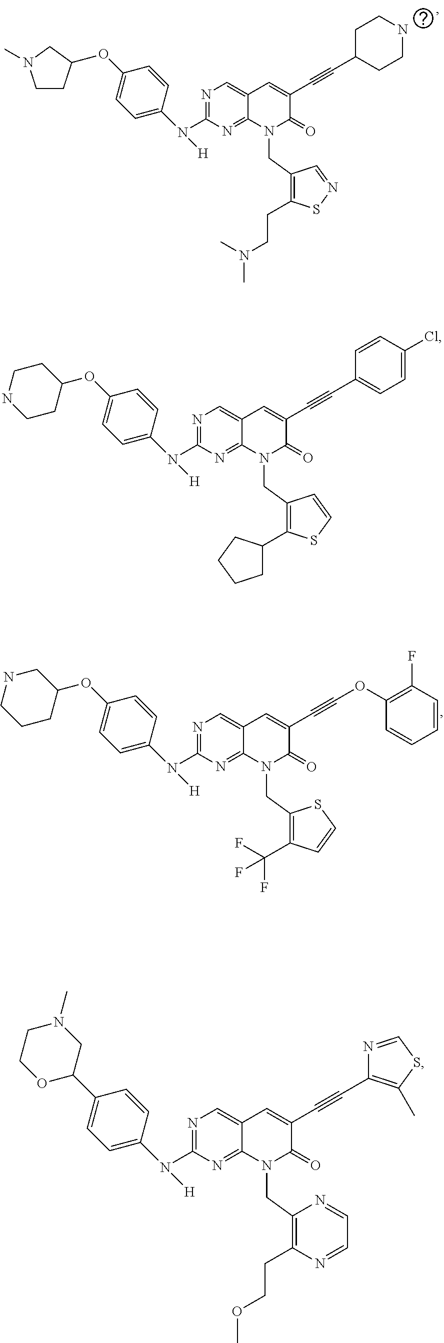 [SMILES: CN(C)CCc1sncc1Cn1c(=O)c(C#CC2CCNCC2)cc2cnc(Nc3ccc(OC4CCN(C)C4)cc3)nc21 ; O=c1c(C#Cc2ccc(Cl)cc2)cc2cnc(Nc3ccc(OC4CCNCC4)cc3)nc2n1Cc1ccsc1C1CCCC1 ; O=c1c(C#COc2ccccc2F)cc2cnc(Nc3ccc(OC4CCCNC4)cc3)nc2n1Cc1sccc1C(F)(F)F ; COCCc1nccnc1Cn1c(=O)c(C#Cc2ncsc2C)cc2cnc(Nc3ccc(C4CN(C)CCO4)cc3)nc21]

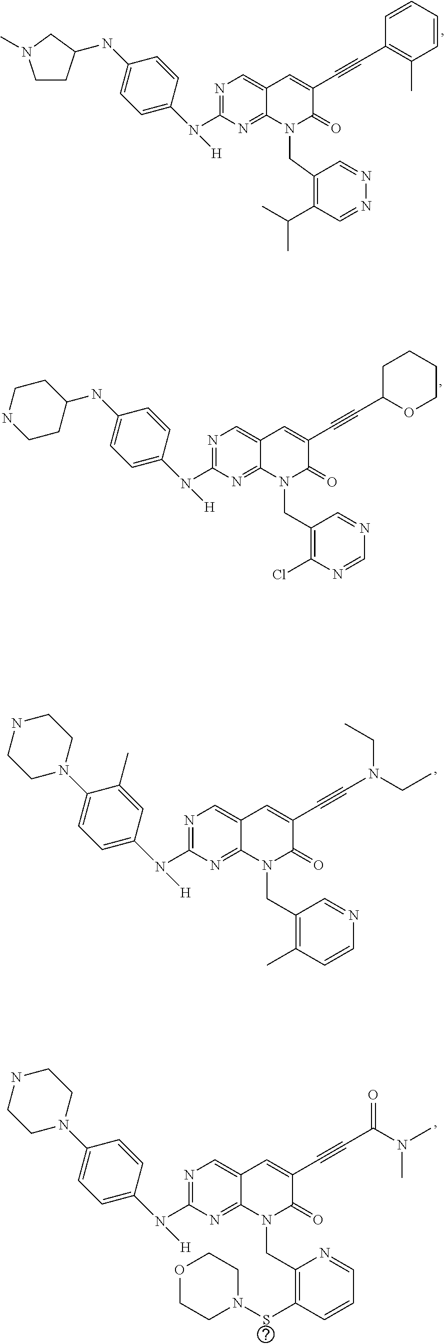 [SMILES: Cc1ccccc1C#Cc1cc2cnc(Nc3ccc(NC4CCN(C)C4)cc3)nc2n(Cc2cnncc2C(C)C)c1=O ; O=c1c(C#CC2CCCCO2)cc2cnc(Nc3ccc(NC4CCNCC4)cc3)nc2n1Cc1cncnc1Cl ; CCN(C#Cc1cc2cnc(Nc3ccc(N4CCNCC4)c(C)c3)nc2n(Cc2cnccc2C)c1=O)CC ; CN(C)C(=O)C#Cc1cc2cnc(Nc3ccc(N4CCNCC4)cc3)nc2n(Cc2ncccc2SN2CCOCC2)c1=O]

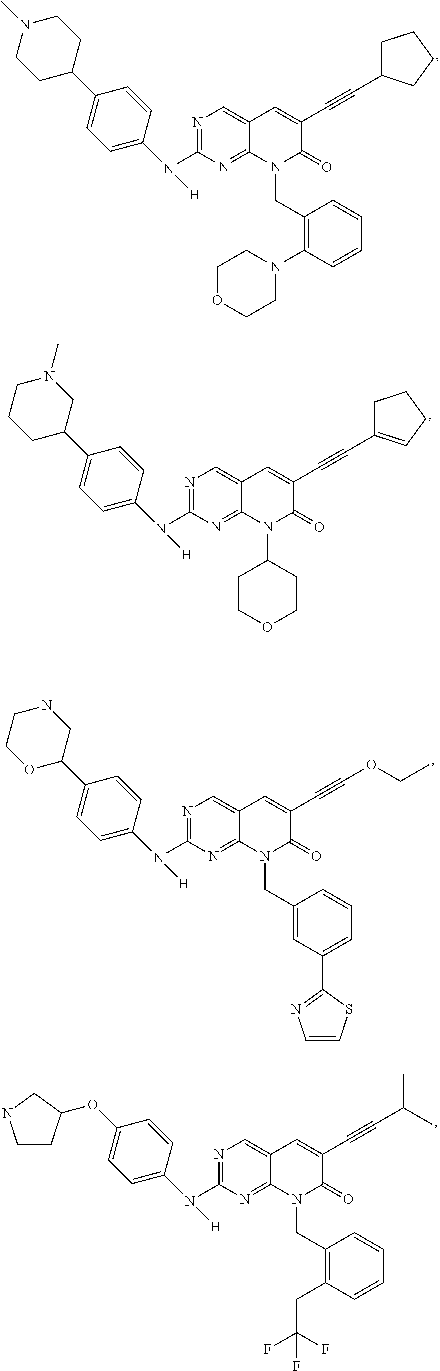 [SMILES: CN1CCC(c2ccc(Nc3ncc4cc(C#CC5CCCC5)c(=O)n(Cc5ccccc5N5CCOCC5)c4n3)cc2)CC1 ; CN1CCCC(c2ccc(Nc3ncc4cc(C#CC5=CCCC5)c(=O)n(C5CCOCC5)c4n3)cc2)C1 ; CCOC#Cc1cc2cnc(Nc3ccc(C4CNCCO4)cc3)nc2n(Cc2cccc(-c3nccs3)c2)c1=O ; CC(C)C#Cc1cc2cnc(Nc3ccc(OC4CCNC4)cc3)nc2n(Cc2ccccc2CC(F)(F)F)c1=O]

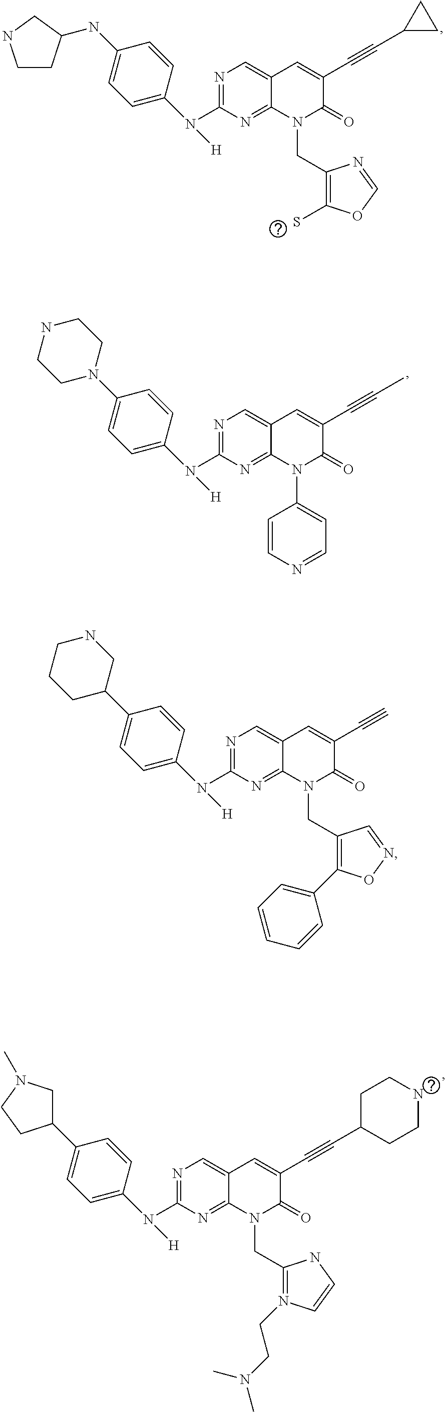

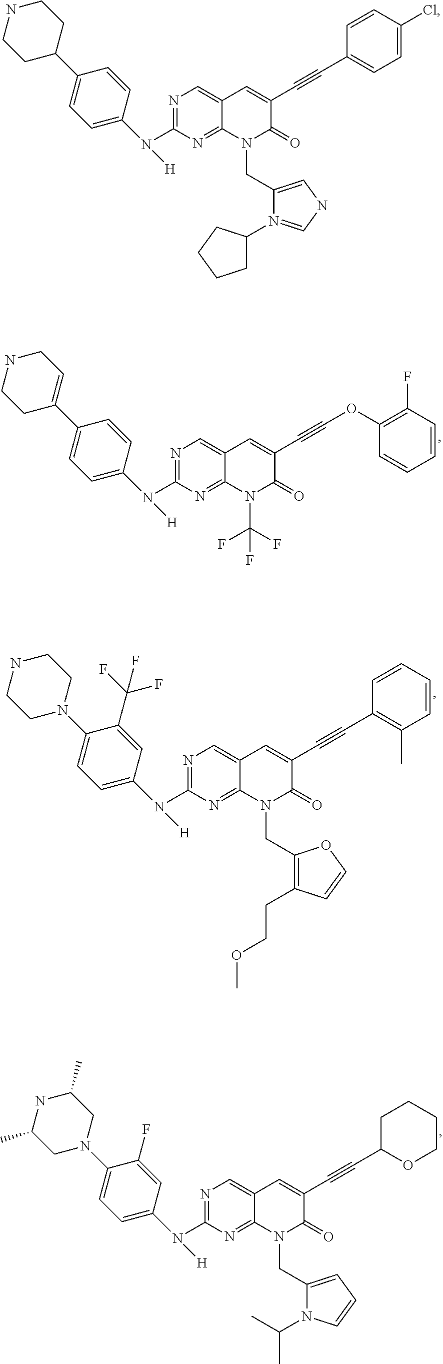

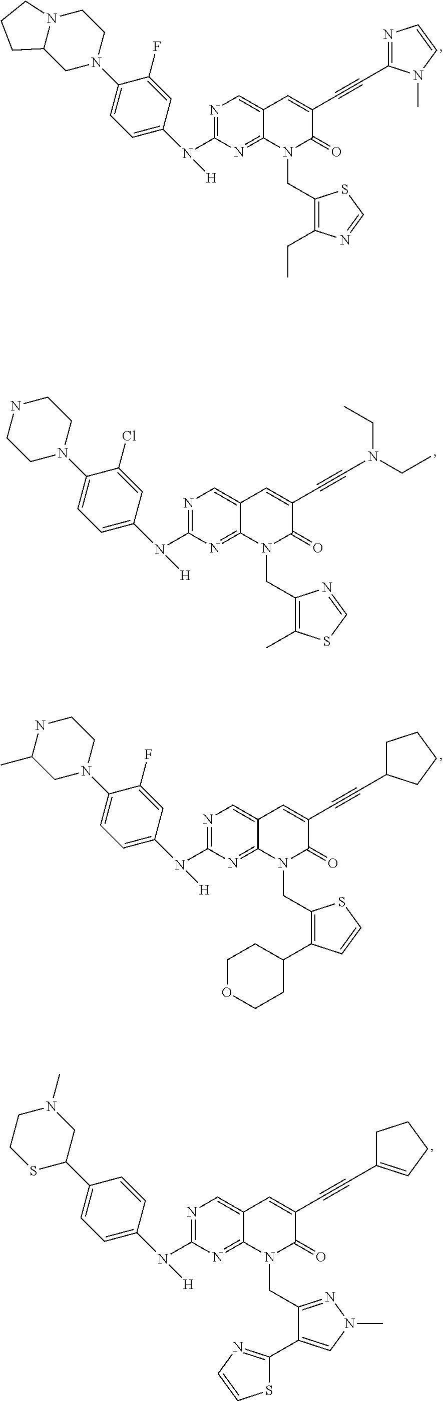

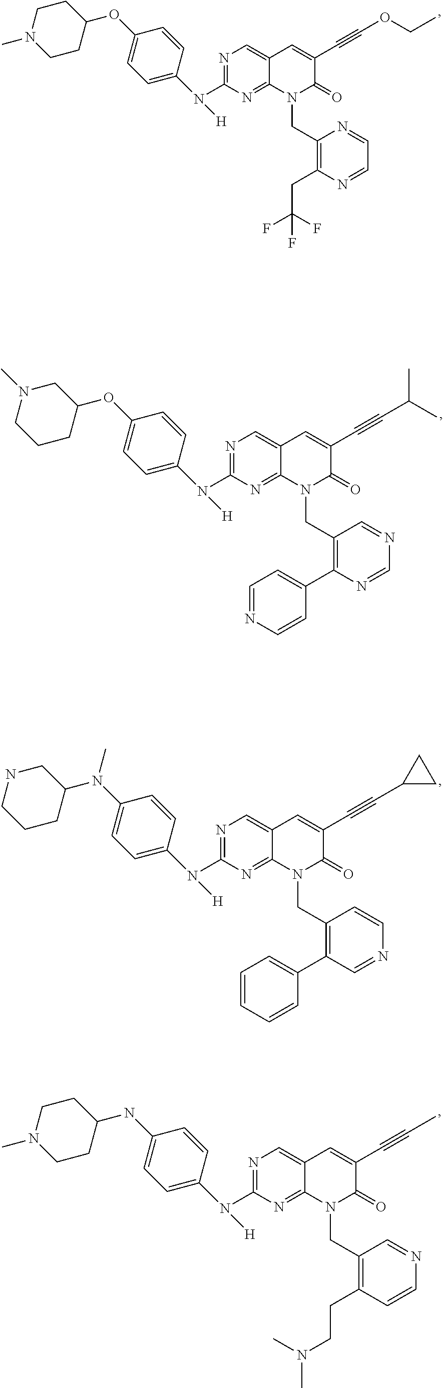

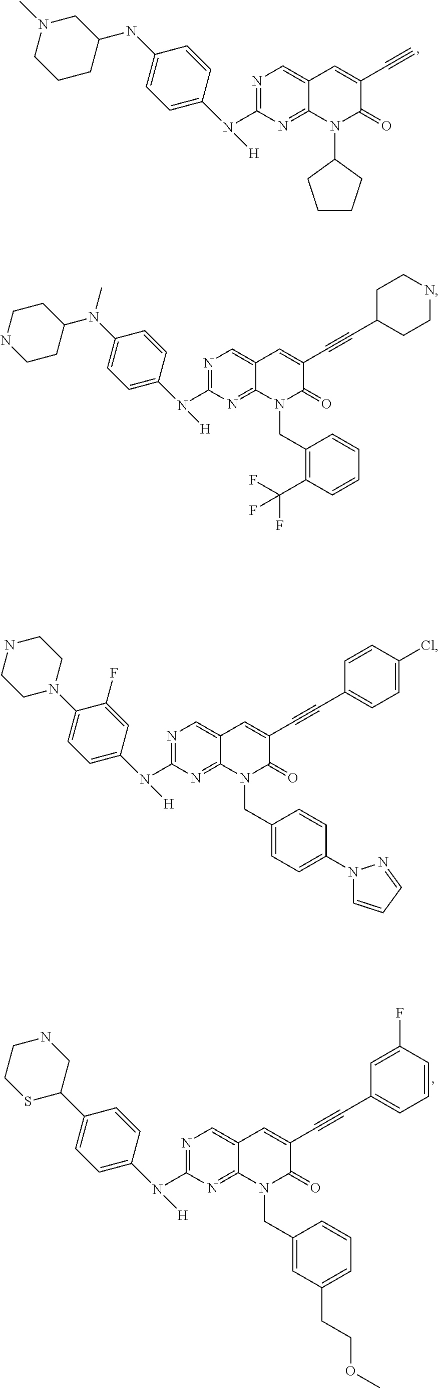

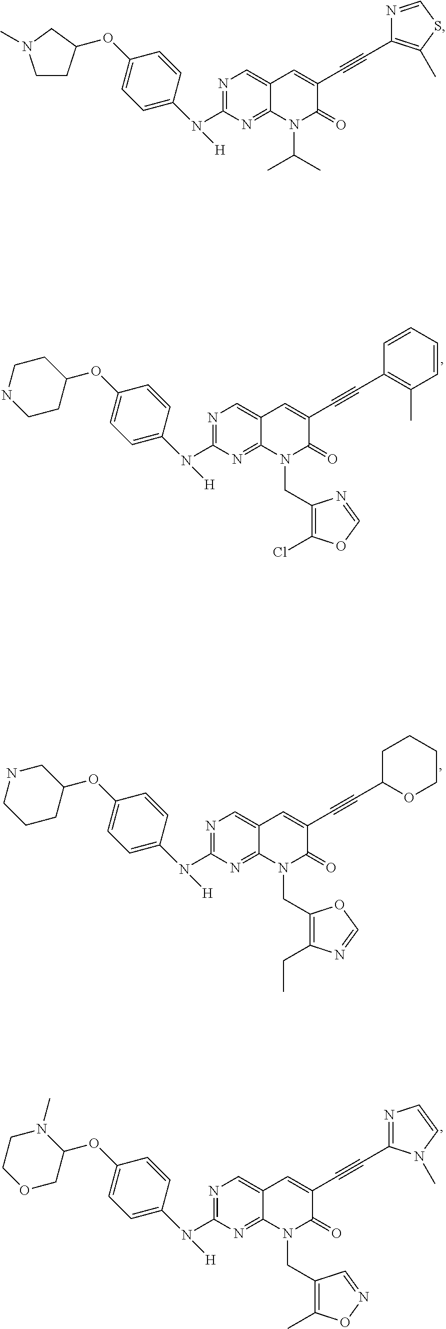 [SMILES: Cc1scnc1C#Cc1cc2cnc(Nc3ccc(OC4CCN(C)C4)cc3)nc2n(C(C)C)c1=O ; Cc1ccccc1C#Cc1cc2cnc(Nc3ccc(OC4CCNCC4)cc3)nc2n(Cc2ncoc2Cl)c1=O ; CCc1ncoc1Cn1c(=O)c(C#CC2CCCCO2)cc2cnc(Nc3ccc(OC4CCCNC4)cc3)nc21 ; Cc1oncc1Cn1c(=O)c(C#Cc2nccn2C)cc2cnc(Nc3ccc(OC4COCCN4C)cc3)nc21]

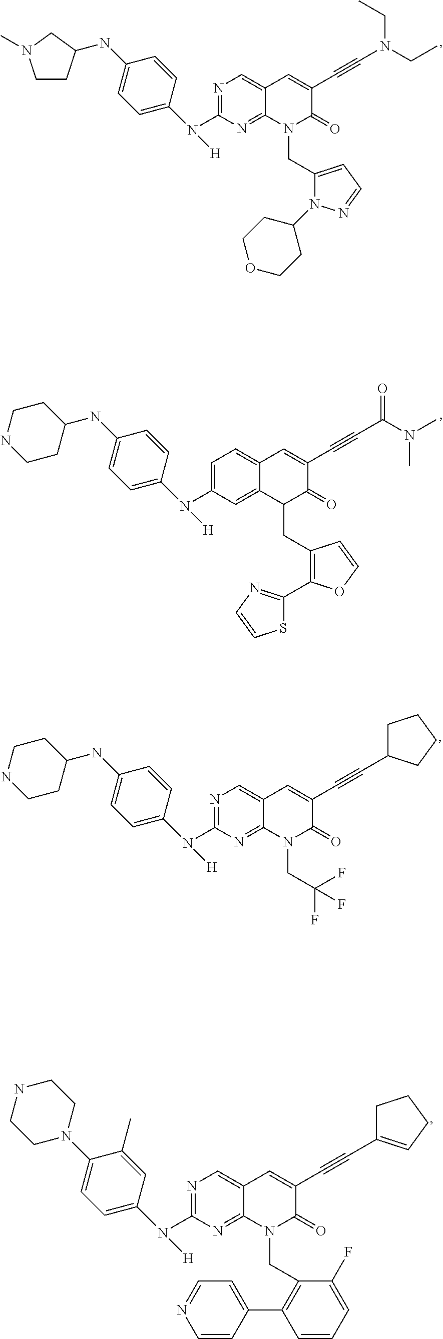 [SMILES: CCN(C#Cc1cc2cnc(Nc3ccc(NC4CCN(C)C4)cc3)nc2n(Cc2ccnn2C2CCOCC2)c1=O)CC ; CN(C)C(=O)C#CC1=Cc2ccc(Nc3ccc(NC4CCNCC4)cc3)cc2C(Cc2ccoc2-c2nccs2)C1=O ; O=c1c(C#CC2CCCC2)cc2cnc(Nc3ccc(NC4CCNCC4)cc3)nc2n1CC(F)(F)F ; Cc1cc(Nc2ncc3cc(C#CC4=CCCC4)c(=O)n(Cc4c(F)cccc4-c4ccncc4)c3n2)ccc1N1CCNCC1]

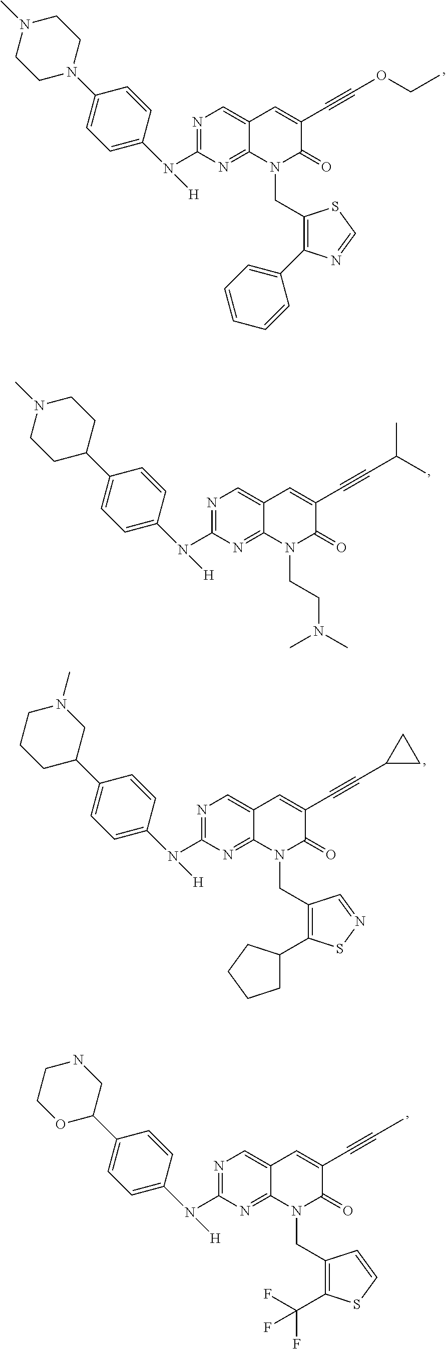

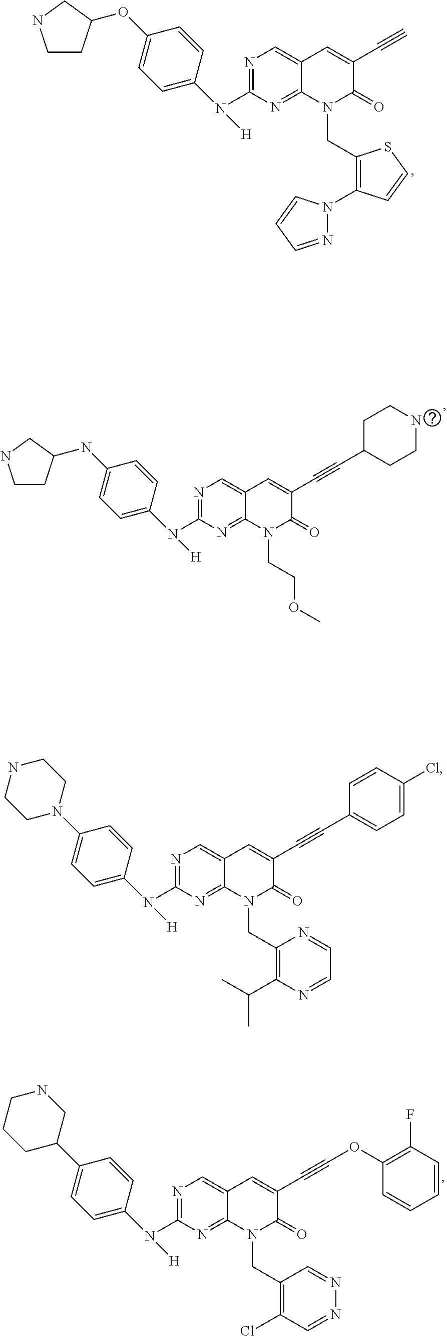 [SMILES: C#Cc1cc2cnc(Nc3ccc(OC4CCNC4)cc3)nc2n(Cc2sccc2-n2cccn2)c1=O ; COCCn1c(=O)c(C#CC2CCNCC2)cc2cnc(Nc3ccc(NC4CCNC4)cc3)nc21 ; CC(C)c1nccnc1Cn1c(=O)c(C#Cc2ccc(Cl)cc2)cc2cnc(Nc3ccc(N4CCNCC4)cc3)nc21 ; O=c1c(C#COc2ccccc2F)cc2cnc(Nc3ccc(C4CCCNC4)cc3)nc2n1Cc1cnncc1Cl]

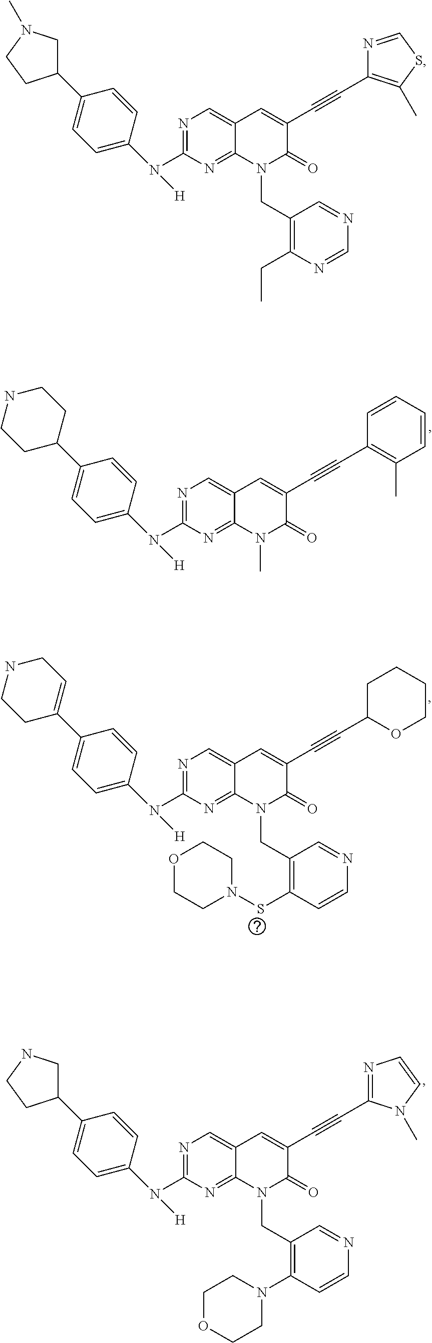 [SMILES: CCc1ncncc1Cn1c(=O)c(C#Cc2ncsc2C)cc2cnc(Nc3ccc(C4CCN(C)C4)cc3)nc21 ; Cc1ccccc1C#Cc1cc2cnc(Nc3ccc(C4CCNCC4)cc3)nc2n(C)c1=O ; O=c1c(C#CC2CCCCO2)cc2cnc(Nc3ccc(C4=CCNCC4)cc3)nc2n1Cc1cnccc1SN1CCOCC1 ; Cn1ccnc1C#Cc1cc2cnc(Nc3ccc(C4CCNC4)cc3)nc2n(Cc2cnccc2N2CCOCC2)c1=O]

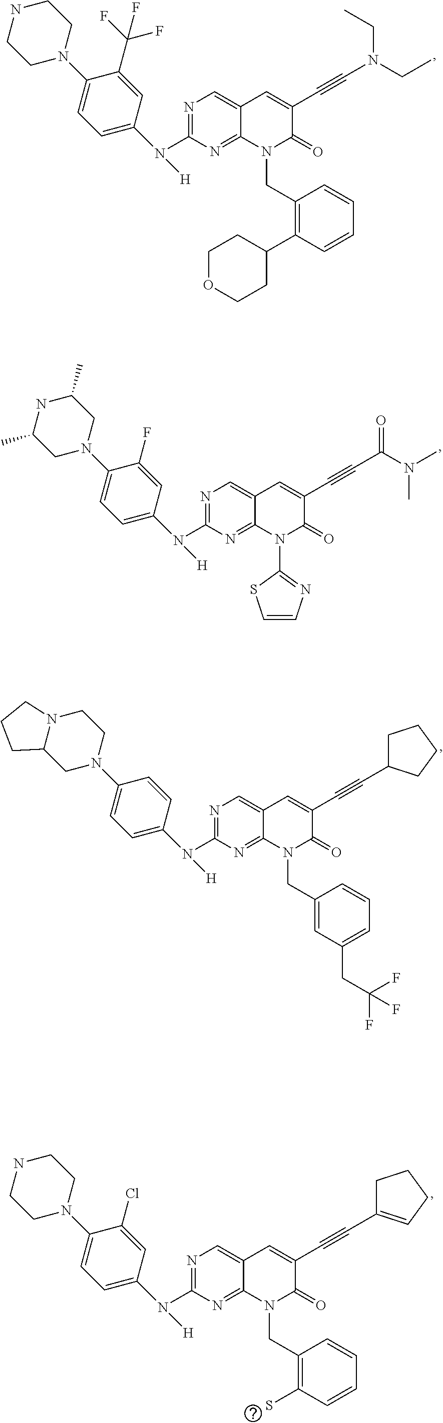

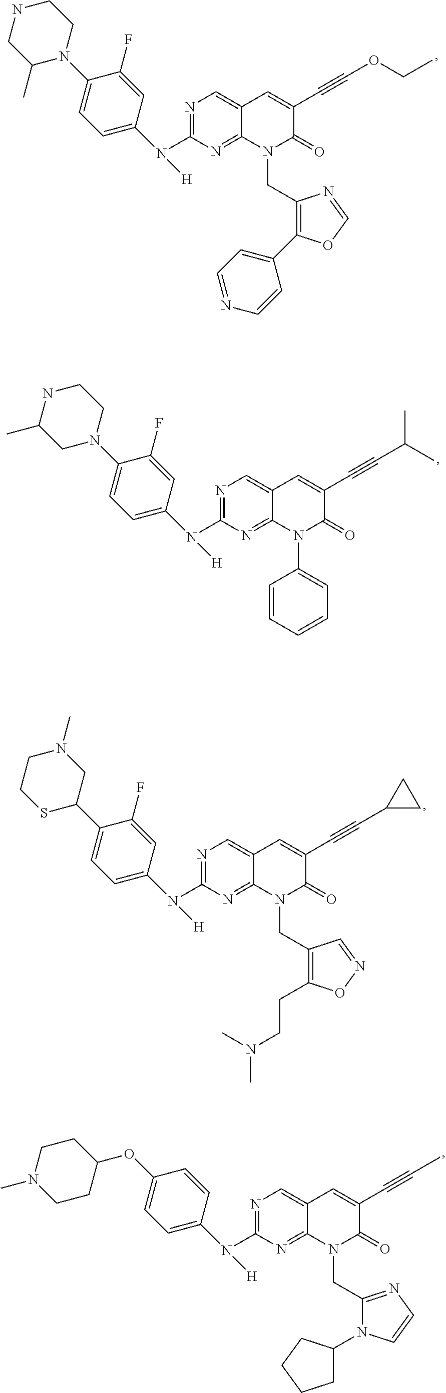

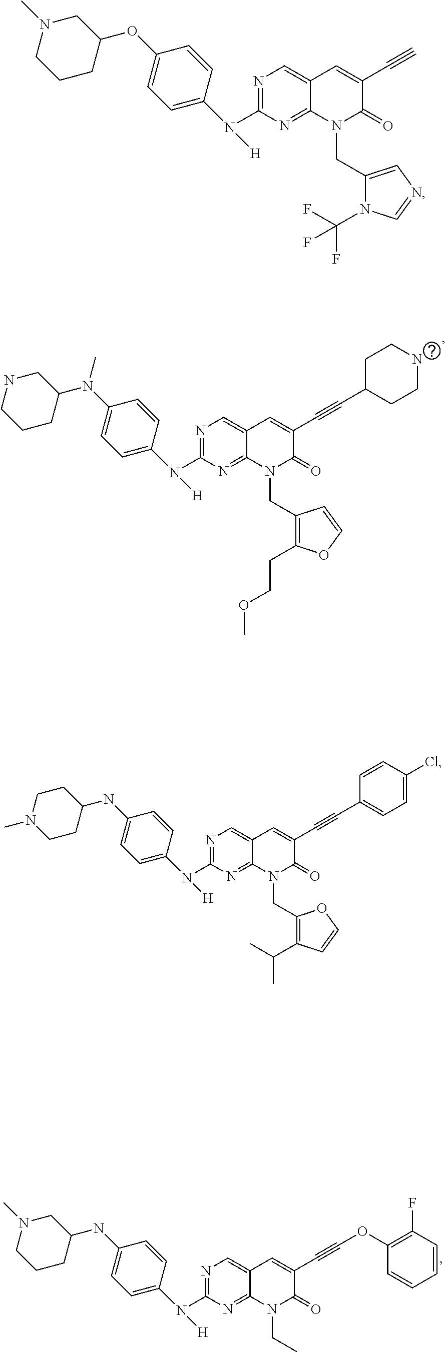 [SMILES: C#Cc1cc2cnc(Nc3ccc(OC4CCCN(C)C4)cc3)nc2n(Cc2cncn2C(F)(F)F)c1=O ; COCCc1occc1Cn1c(=O)c(C#CC2CCNCC2)cc2cnc(Nc3ccc(N(C)C4CCCNC4)cc3)nc21 ; CC(C)c1ccoc1Cn1c(=O)c(C#Cc2ccc(Cl)cc2)cc2cnc(Nc3ccc(NC4CCN(C)CC4)cc3)nc21 ; CCn1c(=O)c(C#COc2ccccc2F)cc2cnc(Nc3ccc(NC4CCCN(C)C4)cc3)nc21]

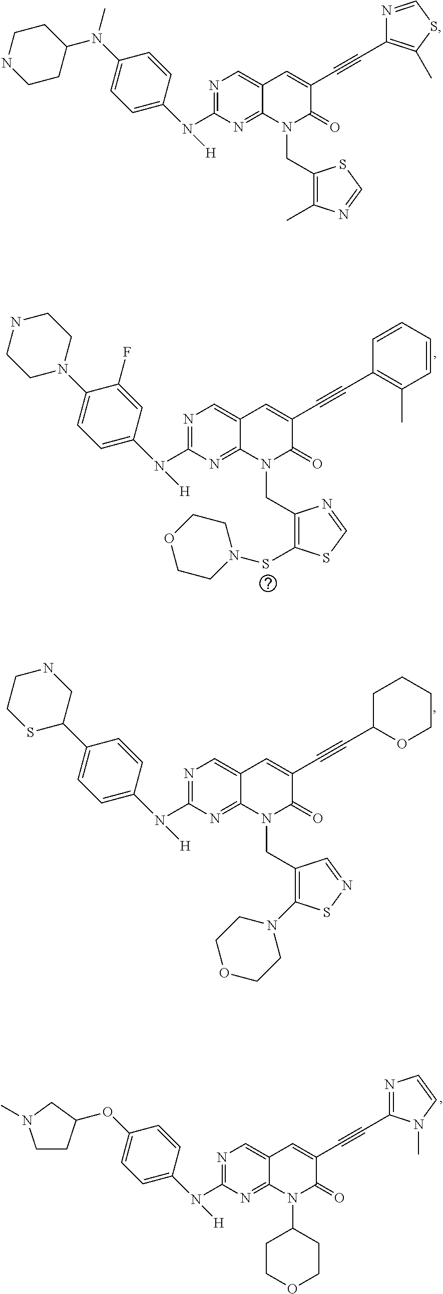 [SMILES: Cc1ncsc1Cn1c(=O)c(C#Cc2ncsc2C)cc2cnc(Nc3ccc(N(C)C4CCNCC4)cc3)nc21 ; Cc1ccccc1C#Cc1cc2cnc(Nc3ccc(N4CCNCC4)c(F)c3)nc2n(Cc2ncsc2SN2CCOCC2)c1=O ; O=c1c(C#CC2CCCCO2)cc2cnc(Nc3ccc(C4CNCCS4)cc3)nc2n1Cc1cnsc1N1CCOCC1 ; CN1CCC(Oc2ccc(Nc3ncc4cc(C#Cc5nccn5C)c(=O)n(C5CCOCC5)c4n3)cc2)C1]

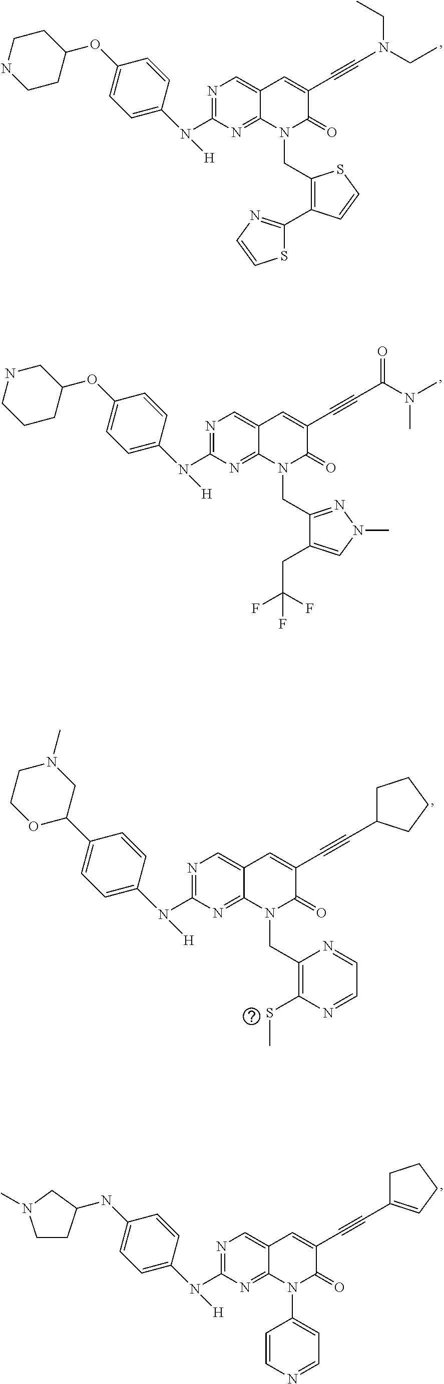

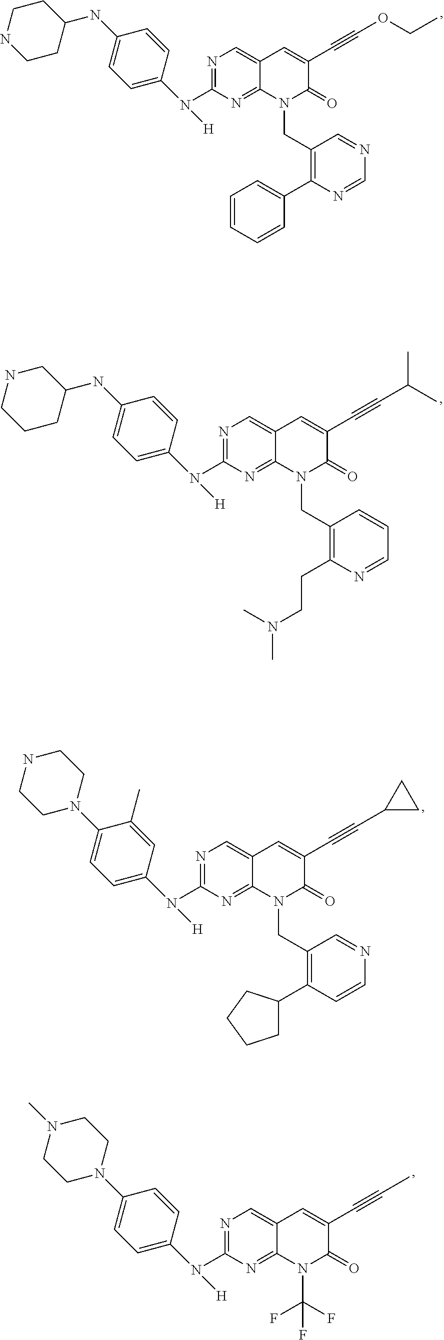

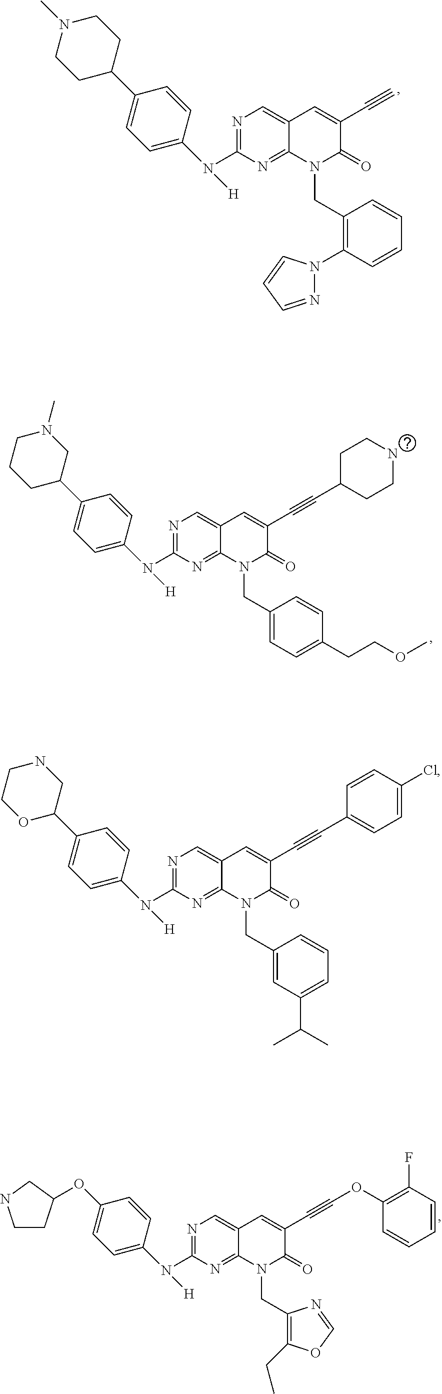 [SMILES: C#Cc1cc2cnc(Nc3ccc(C4CCN(C)CC4)cc3)nc2n(Cc2ccccc2-n2cccn2)c1=O ; COCCc1ccc(Cn2c(=O)c(C#CC3CCNCC3)cc3cnc(Nc4ccc(C5CCCN(C)C5)cc4)nc32)cc1 ; CC(C)c1cccc(Cn2c(=O)c(C#Cc3ccc(Cl)cc3)cc3cnc(Nc4ccc(C5CNCCO5)cc4)nc32)c1 ; CCc1ocnc1Cn1c(=O)c(C#COc2ccccc2F)cc2cnc(Nc3ccc(OC4CCNC4)cc3)nc21]

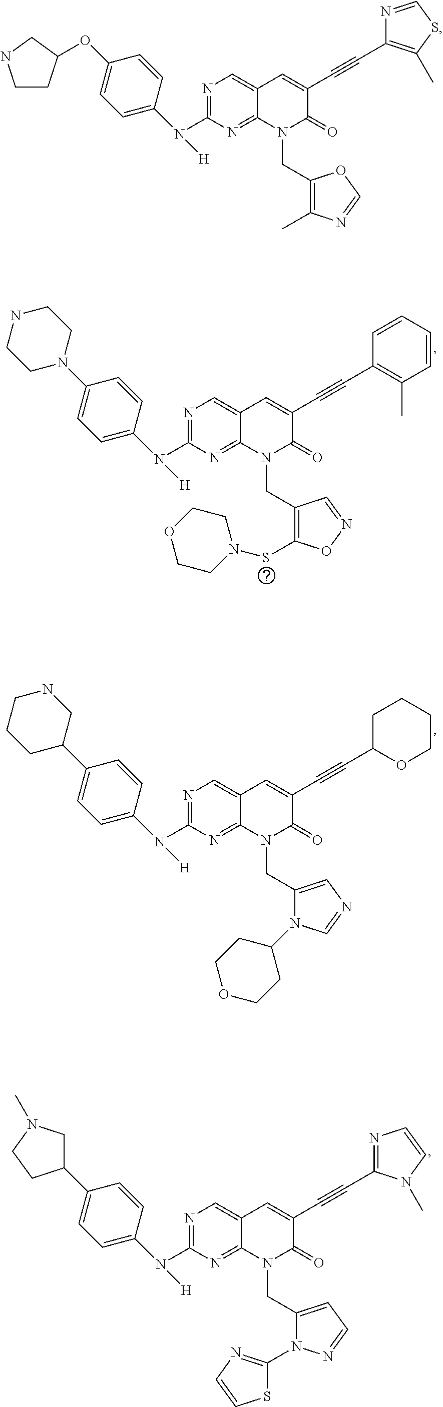 [SMILES: Cc1ncoc1Cn1c(=O)c(C#Cc2ncsc2C)cc2cnc(Nc3ccc(OC4CCNC4)cc3)nc21 ; Cc1ccccc1C#Cc1cc2cnc(Nc3ccc(N4CCNCC4)cc3)nc2n(Cc2cnoc2SN2CCOCC2)c1=O ; O=c1c(C#CC2CCCCO2)cc2cnc(Nc3ccc(C4CCCNC4)cc3)nc2n1Cc1cncn1C1CCOCC1 ; CN1CCC(c2ccc(Nc3ncc4cc(C#Cc5nccn5C)c(=O)n(Cc5ccnn5-c5nccs5)c4n3)cc2)C1]

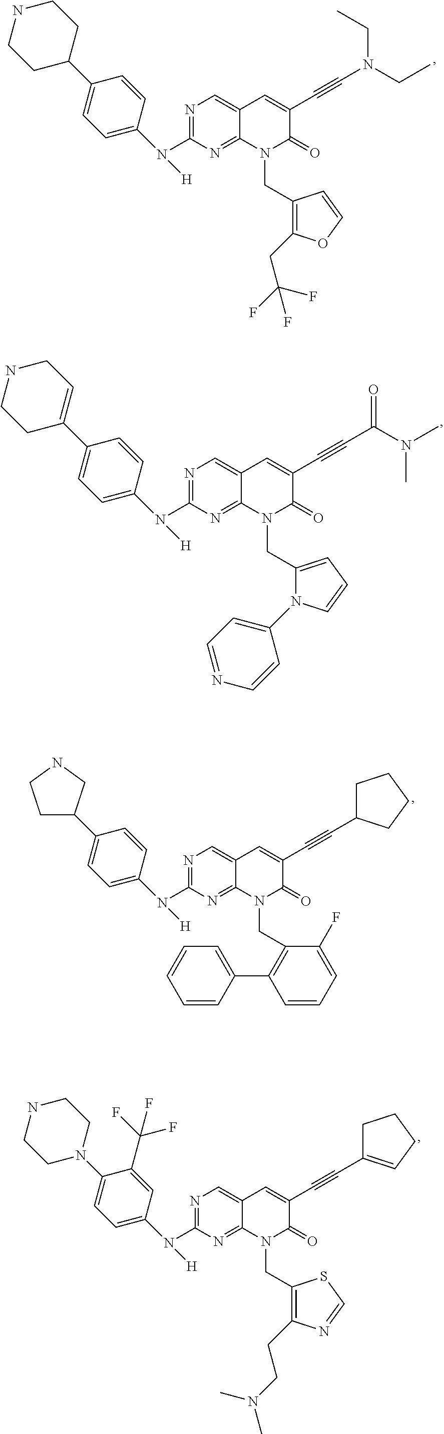 [SMILES: CCN(C#Cc1cc2cnc(Nc3ccc(C4CCNCC4)cc3)nc2n(Cc2ccoc2CC(F)(F)F)c1=O)CC ; CN(C)C(=O)C#Cc1cc2cnc(Nc3ccc(C4=CCNCC4)cc3)nc2n(Cc2cccn2-c2ccncc2)c1=O ; O=c1c(C#CC2CCCC2)cc2cnc(Nc3ccc(C4CCNC4)cc3)nc2n1Cc1c(F)cccc1-c1ccccc1 ; CN(C)CCc1ncsc1Cn1c(=O)c(C#CC2=CCCC2)cc2cnc(Nc3ccc(N4CCNCC4)c(C(F)(F)F)c3)nc21]

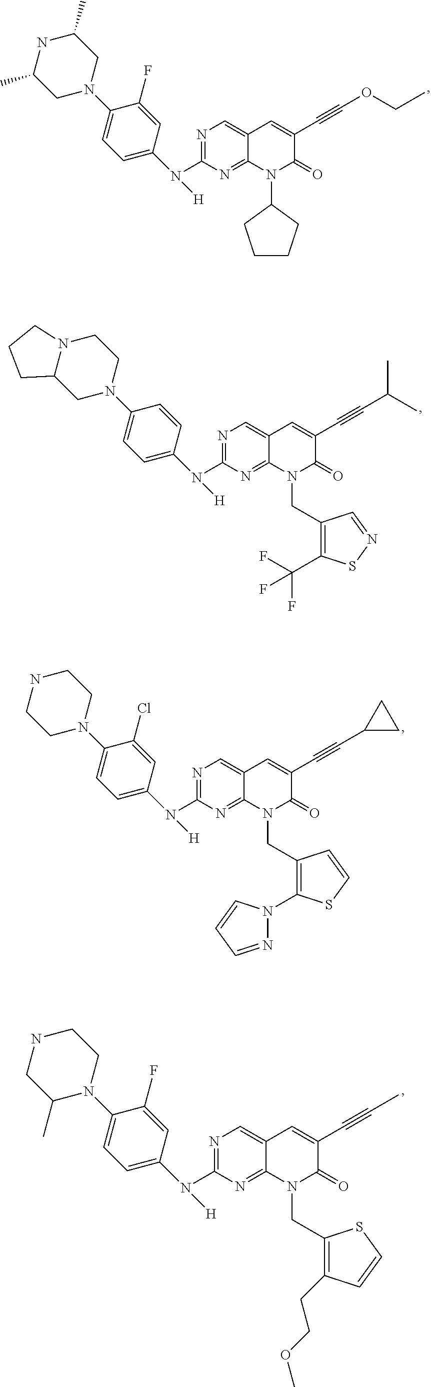 [SMILES: CCOC#Cc1cc2cnc(Nc3ccc(N4C[C@@H](C)N[C@@H](C)C4)c(F)c3)nc2n(C2CCCC2)c1=O ; CC(C)C#Cc1cc2cnc(Nc3ccc(N4CCN5CCCC5C4)cc3)nc2n(Cc2cnsc2C(F)(F)F)c1=O ; O=c1c(C#CC2CC2)cc2cnc(Nc3ccc(N4CCNCC4)c(Cl)c3)nc2n1Cc1ccsc1-n1cccn1 ; CC#Cc1cc2cnc(Nc3ccc(N4CCNCC4C)c(F)c3)nc2n(Cc2sccc2CCOC)c1=O]

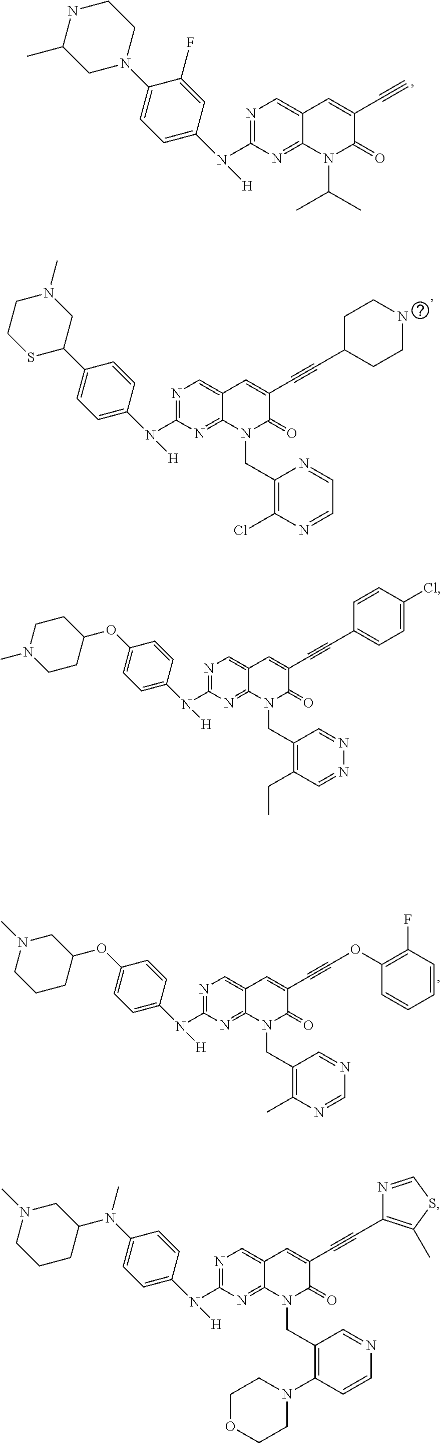

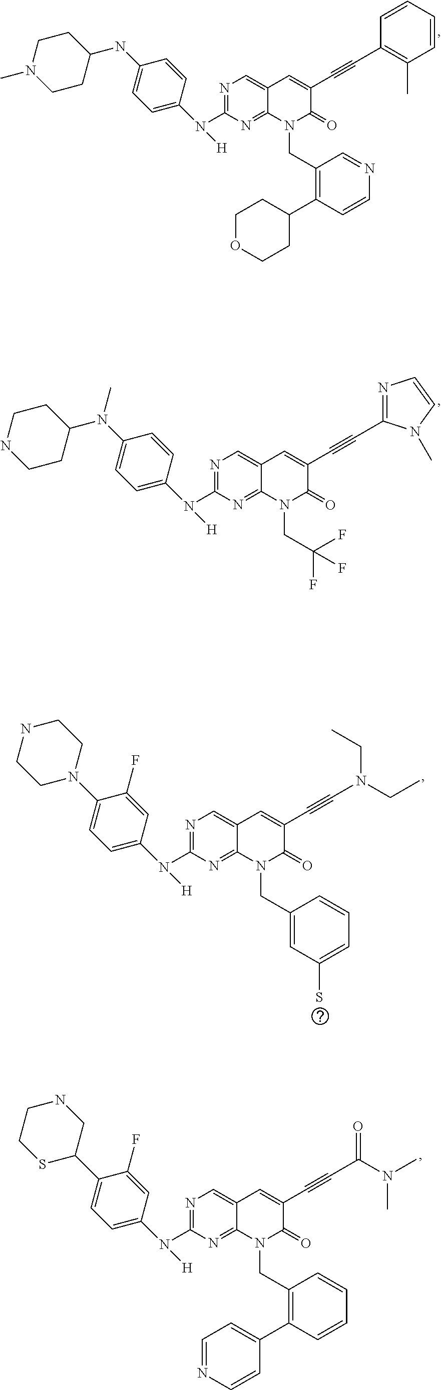 [SMILES: Cc1ccccc1C#Cc1cc2cnc(Nc3ccc(NC4CCN(C)CC4)cc3)nc2n(Cc2cnccc2C2CCOCC2)c1=O ; CN(c1ccc(Nc2ncc3cc(C#Cc4nccn4C)c(=O)n(CC(F)(F)F)c3n2)cc1)C1CCNCC1 ; CCN(C#Cc1cc2cnc(Nc3ccc(N4CCNCC4)c(F)c3)nc2n(Cc2cccc(S)c2)c1=O)CC ; CN(C)C(=O)C#Cc1cc2cnc(Nc3ccc(C4CNCCS4)c(F)c3)nc2n(Cc2ccccc2-c2ccncc2)c1=O]

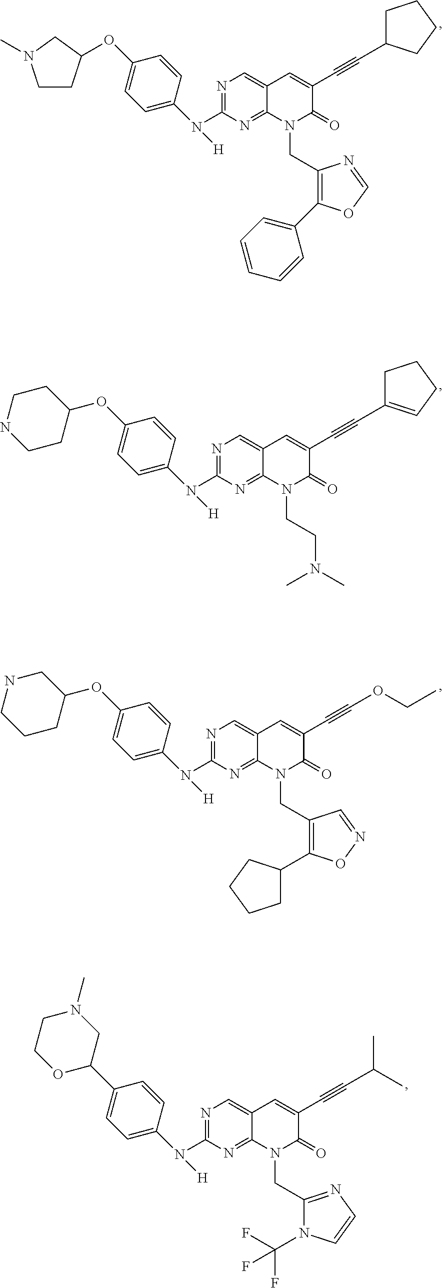 [SMILES: CN1CCC(Oc2ccc(Nc3ncc4cc(C#CC5CCCC5)c(=O)n(Cc5ncoc5-c5ccccc5)c4n3)cc2)C1 ; CN(C)CCn1c(=O)c(C#CC2=CCCC2)cc2cnc(Nc3ccc(OC4CCNCC4)cc3)nc21 ; CCOC#Cc1cc2cnc(Nc3ccc(OC4CCCNC4)cc3)nc2n(Cc2cnoc2C2CCCC2)c1=O ; CC(C)C#Cc1cc2cnc(Nc3ccc(C4CN(C)CCO4)cc3)nc2n(Cc2nccn2C(F)(F)F)c1=O]

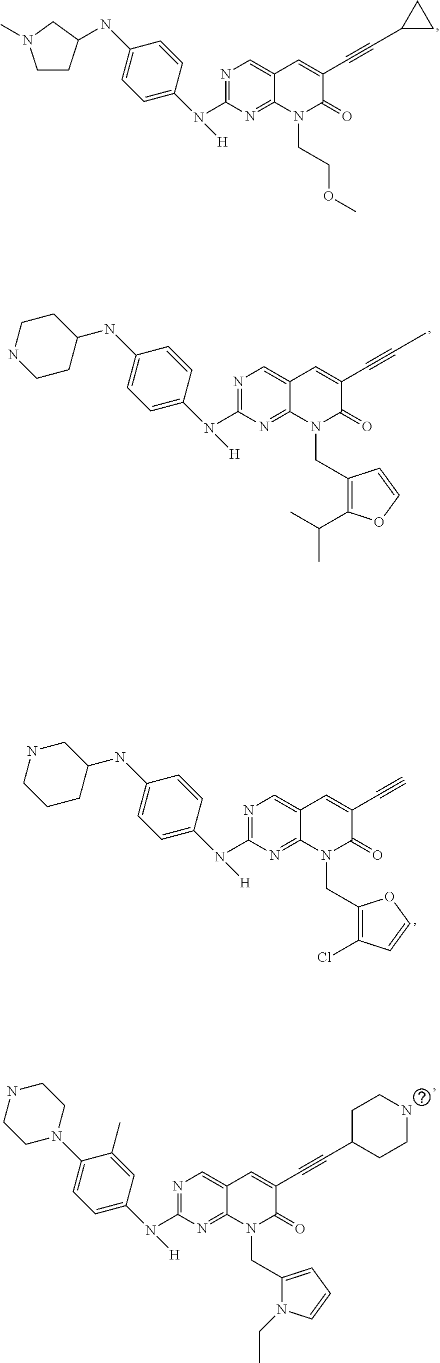 [SMILES: COCCn1c(=O)c(C#CC2CC2)cc2cnc(Nc3ccc(NC4CCN(C)C4)cc3)nc21 ; CC#Cc1cc2cnc(Nc3ccc(NC4CCNCC4)cc3)nc2n(Cc2ccoc2C(C)C)c1=O ; C#Cc1cc2cnc(Nc3ccc(NC4CCCNC4)cc3)nc2n(Cc2occc2Cl)c1=O ; CCn1cccc1Cn1c(=O)c(C#CC2CCNCC2)cc2cnc(Nc3ccc(N4CCNCC4)c(C)c3)nc21]

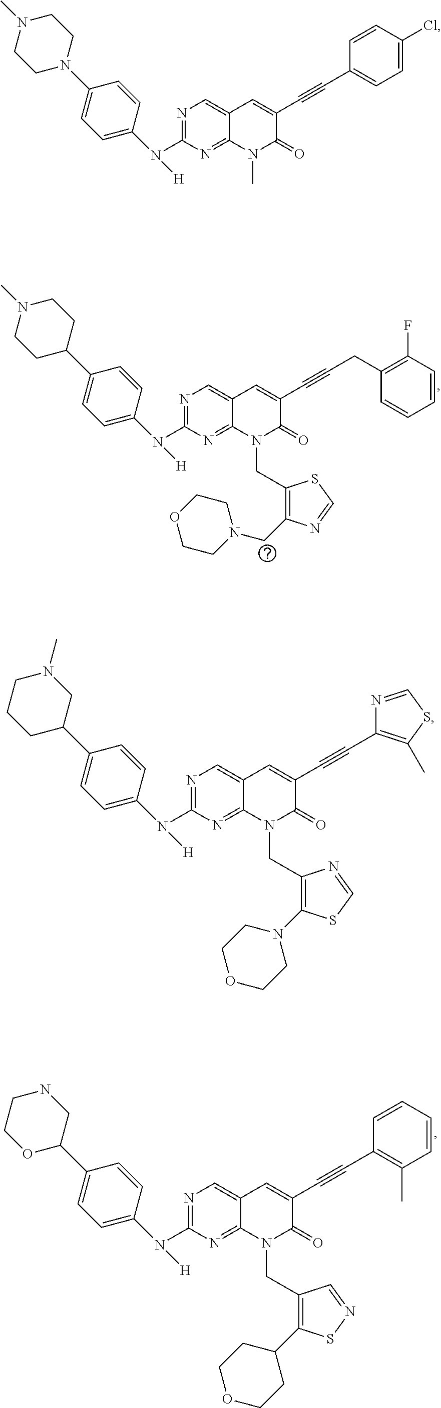 [SMILES: CN1CCN(c2ccc(Nc3ncc4cc(C#Cc5ccc(Cl)cc5)c(=O)n(C)c4n3)cc2)CC1 ; CN1CCC(c2ccc(Nc3ncc4cc(C#CCc5ccccc5F)c(=O)n(Cc5scnc5CN5CCOCC5)c4n3)cc2)CC1 ; Cc1scnc1C#Cc1cc2cnc(Nc3ccc(C4CCCN(C)C4)cc3)nc2n(Cc2ncsc2N2CCOCC2)c1=O ; Cc1ccccc1C#Cc1cc2cnc(Nc3ccc(C4CNCCO4)cc3)nc2n(Cc2cnsc2C2CCOCC2)c1=O]

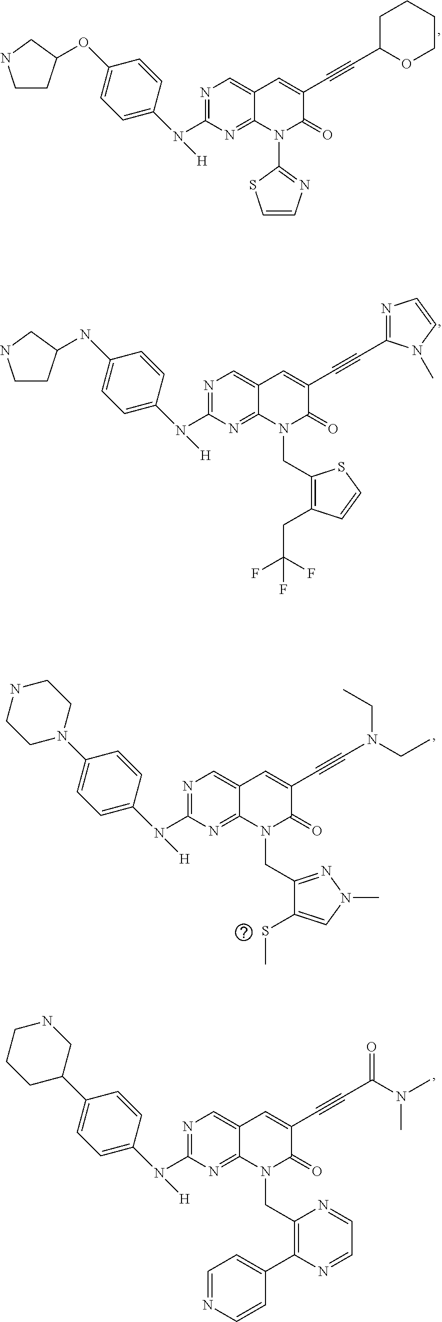 [SMILES: O=c1c(C#CC2CCCCO2)cc2cnc(Nc3ccc(OC4CCNC4)cc3)nc2n1-c1nccs1 ; Cn1ccnc1C#Cc1cc2cnc(Nc3ccc(NC4CCNC4)cc3)nc2n(Cc2sccc2CC(F)(F)F)c1=O ; CCN(C#Cc1cc2cnc(Nc3ccc(N4CCNCC4)cc3)nc2n(Cc2nn(C)cc2SC)c1=O)CC ; CN(C)C(=O)C#Cc1cc2cnc(Nc3ccc(C4CCCNC4)cc3)nc2n(Cc2nccnc2-c2ccncc2)c1=O]

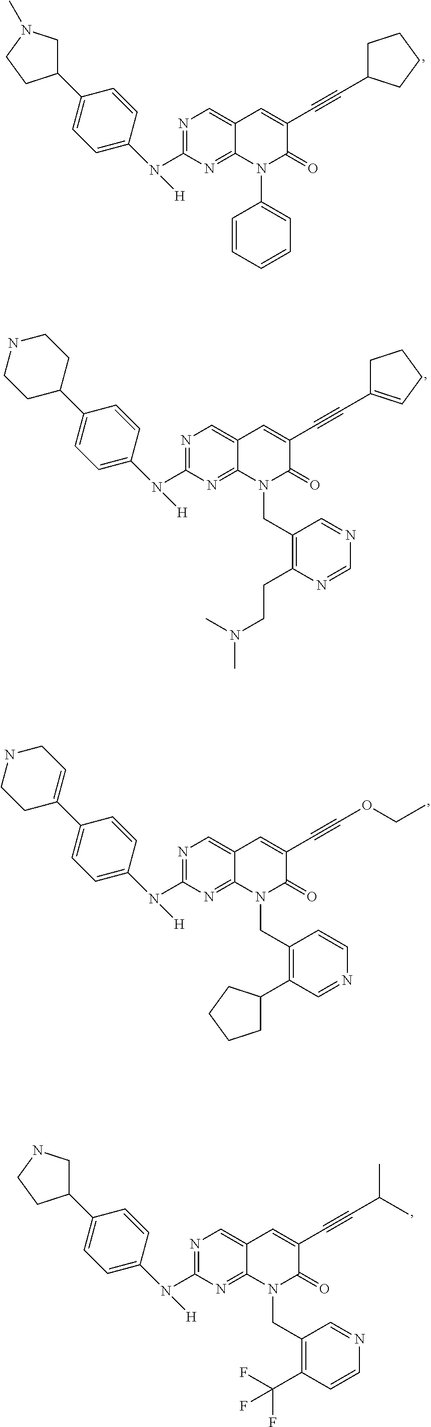 [SMILES: CN1CCC(c2ccc(Nc3ncc4cc(C#CC5CCCC5)c(=O)n(-c5ccccc5)c4n3)cc2)C1 ; CN(C)CCc1ncncc1Cn1c(=O)c(C#CC2=CCCC2)cc2cnc(Nc3ccc(C4CCNCC4)cc3)nc21 ; CCOC#Cc1cc2cnc(Nc3ccc(C4=CCNCC4)cc3)nc2n(Cc2ccncc2C2CCCC2)c1=O ; CC(C)C#Cc1cc2cnc(Nc3ccc(C4CCNC4)cc3)nc2n(Cc2cnccc2C(F)(F)F)c1=O]

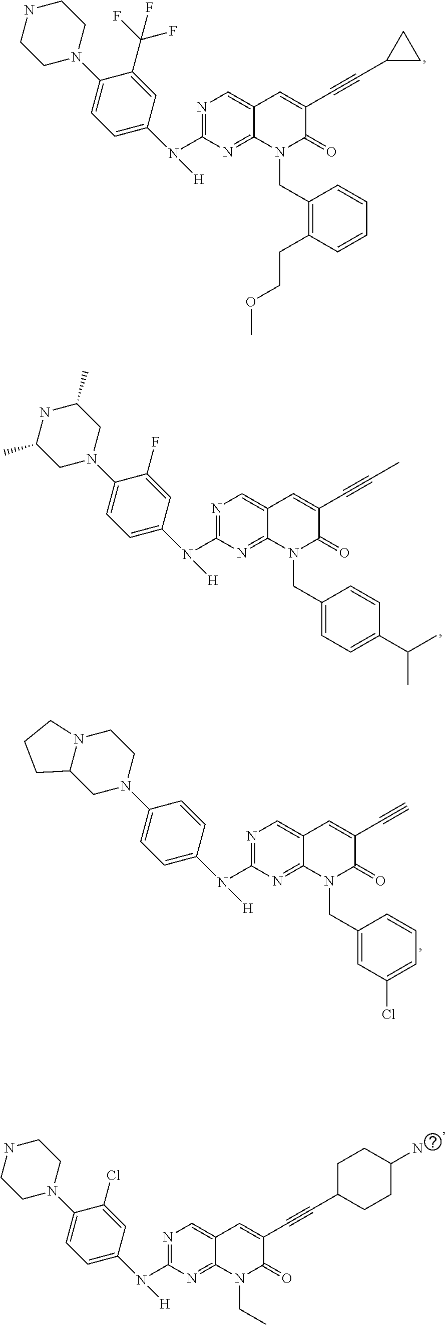 [SMILES: COCCc1ccccc1Cn1c(=O)c(C#CC2CC2)cc2cnc(Nc3ccc(N4CCNCC4)c(C(F)(F)F)c3)nc21 ; CC#Cc1cc2cnc(Nc3ccc(N4C[C@@H](C)N[C@@H](C)C4)c(F)c3)nc2n(Cc2ccc(C(C)C)cc2)c1=O ; C#Cc1cc2cnc(Nc3ccc(N4CCN5CCCC5C4)cc3)nc2n(Cc2cccc(Cl)c2)c1=O ; CCn1c(=O)c(C#CC2CCC(N)CC2)cc2cnc(Nc3ccc(N4CCNCC4)c(Cl)c3)nc21]

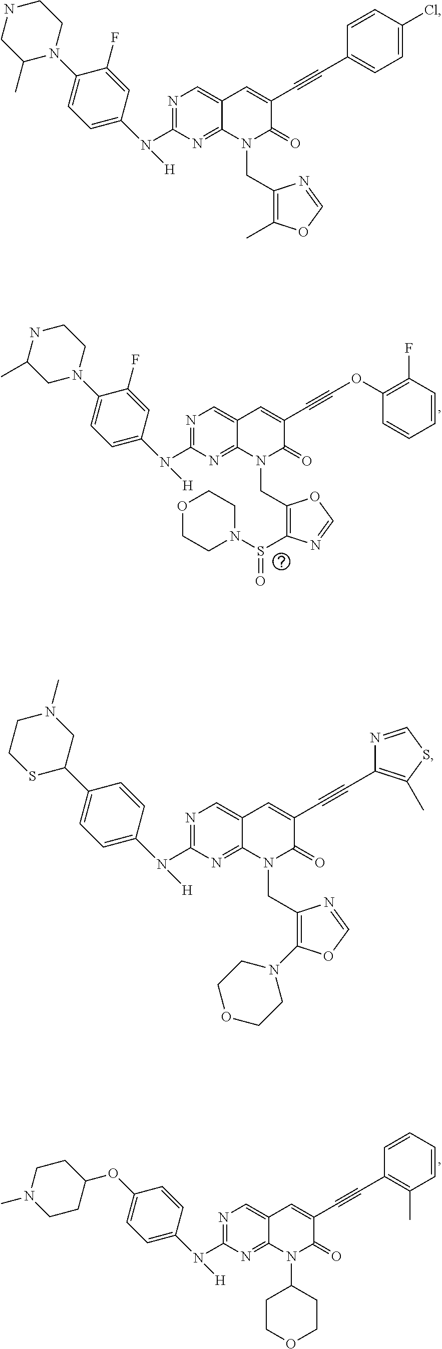 [SMILES: Cc1ocnc1Cn1c(=O)c(C#Cc2ccc(Cl)cc2)cc2cnc(Nc3ccc(N4CCNCC4C)c(F)c3)nc21 ; CC1CN(c2ccc(Nc3ncc4cc(C#COc5ccccc5F)c(=O)n(Cc5ocnc5S(=O)N5CCOCC5)c4n3)cc2F)CCN1 ; Cc1scnc1C#Cc1cc2cnc(Nc3ccc(C4CN(C)CCS4)cc3)nc2n(Cc2ncoc2N2CCOCC2)c1=O ; Cc1ccccc1C#Cc1cc2cnc(Nc3ccc(OC4CCN(C)CC4)cc3)nc2n(C2CCOCC2)c1=O]

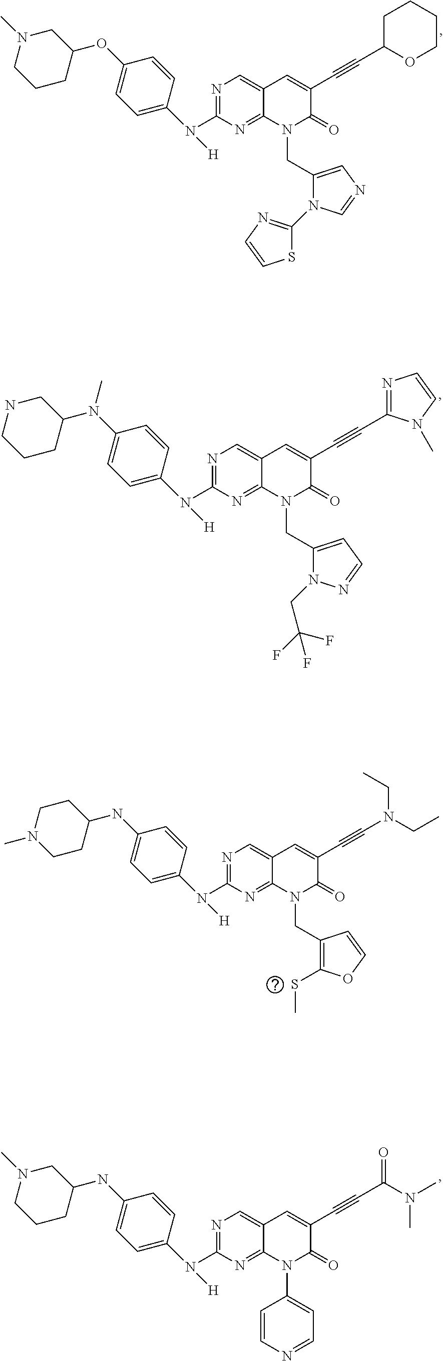

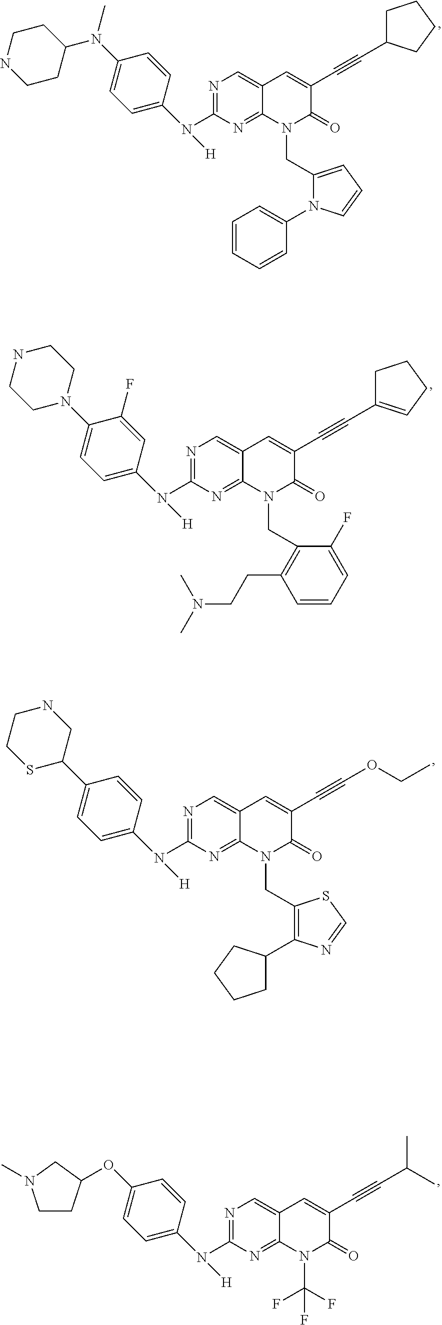

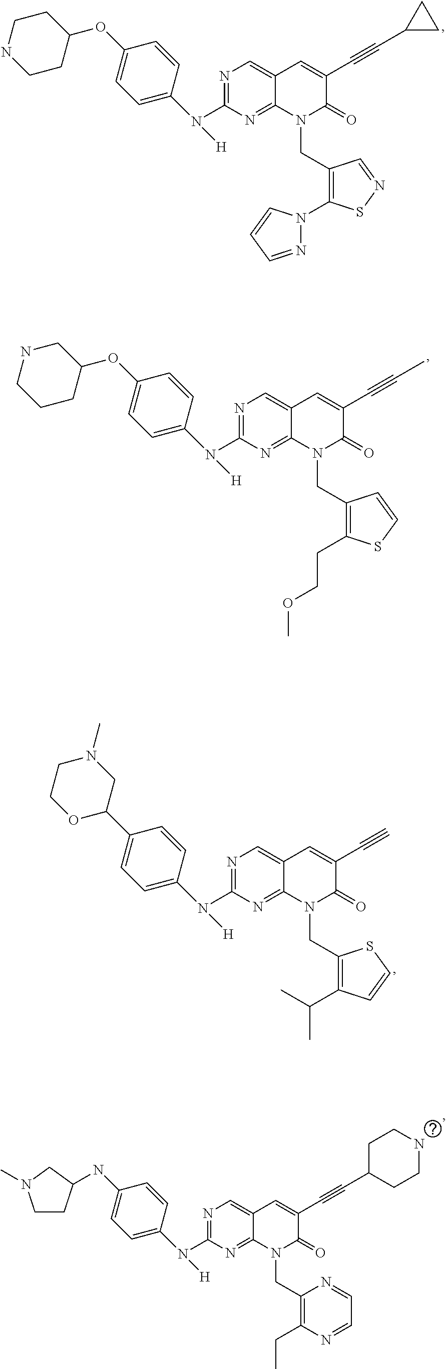

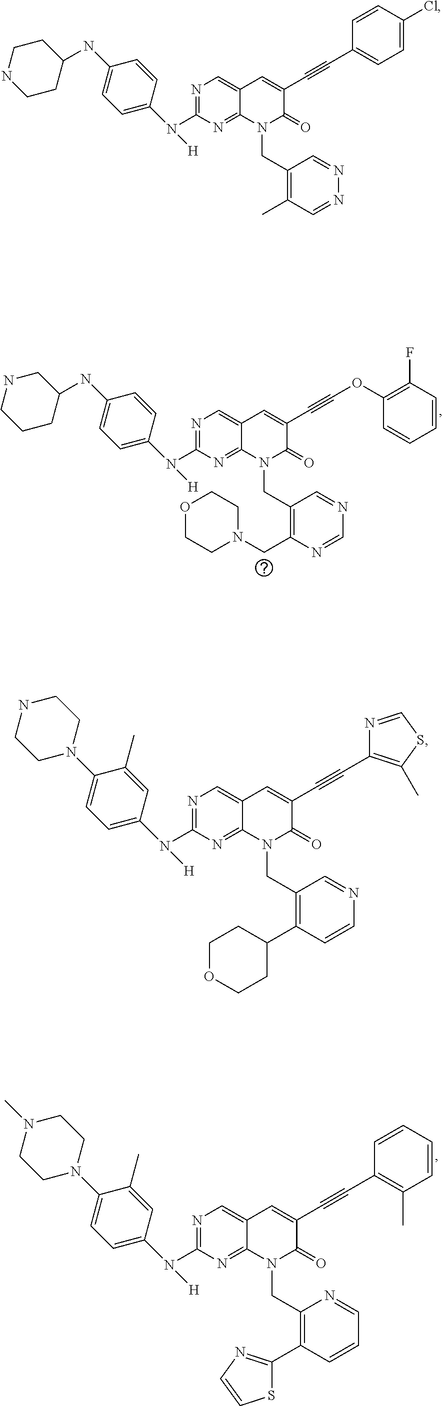 [SMILES: Cc1cnncc1Cn1c(=O)c(C#Cc2ccc(Cl)cc2)cc2cnc(Nc3ccc(NC4CCNCC4)cc3)nc21 ; O=c1c(C#COc2ccccc2F)cc2cnc(Nc3ccc(NC4CCCNC4)cc3)nc2n1Cc1cncnc1CN1CCOCC1 ; Cc1cc(Nc2ncc3cc(C#Cc4ncsc4C)c(=O)n(Cc4cnccc4C4CCOCC4)c3n2)ccc1N1CCNCC1 ; Cc1ccccc1C#Cc1cc2cnc(Nc3ccc(N4CCN(C)CC4)c(C)c3)nc2n(Cc2ncccc2-c2nccs2)c1=O]

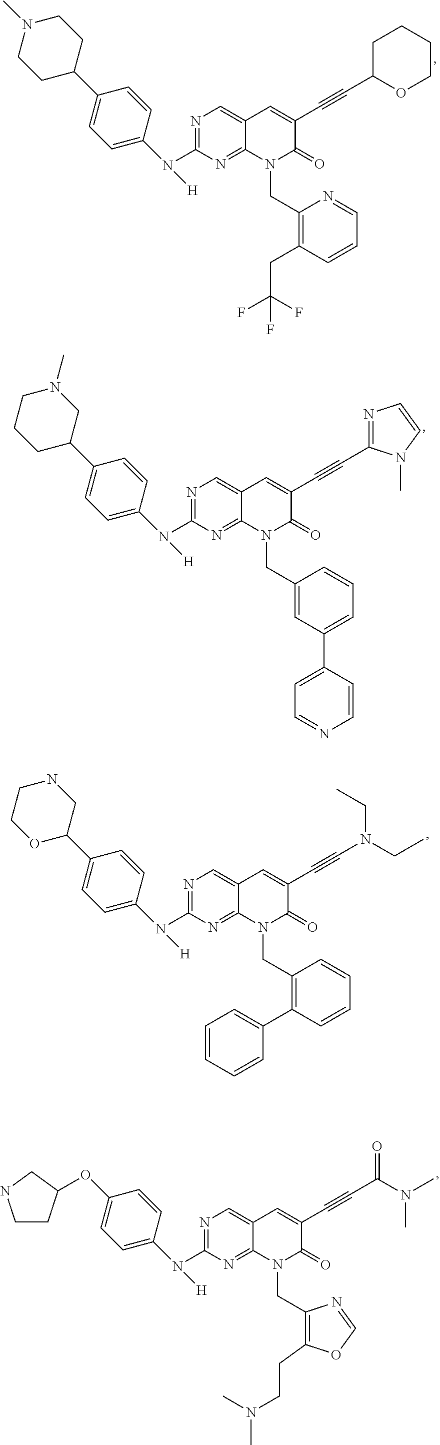 [SMILES: CN1CCC(c2ccc(Nc3ncc4cc(C#CC5CCCCO5)c(=O)n(Cc5ncccc5CC(F)(F)F)c4n3)cc2)CC1 ; CN1CCCC(c2ccc(Nc3ncc4cc(C#Cc5nccn5C)c(=O)n(Cc5cccc(-c6ccncc6)c5)c4n3)cc2)C1 ; CCN(C#Cc1cc2cnc(Nc3ccc(C4CNCCO4)cc3)nc2n(Cc2ccccc2-c2ccccc2)c1=O)CC ; CN(C)CCc1ocnc1Cn1c(=O)c(C#CC(=O)N(C)C)cc2cnc(Nc3ccc(OC4CCNC4)cc3)nc21]

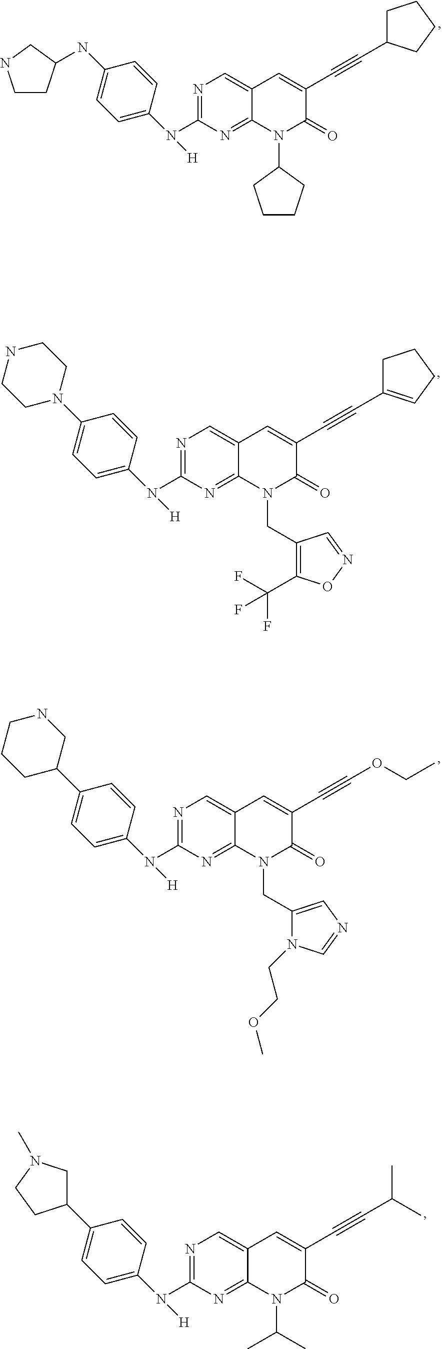 [SMILES: O=c1c(C#CC2CCCC2)cc2cnc(Nc3ccc(NC4CCNC4)cc3)nc2n1C1CCCC1 ; O=c1c(C#CC2=CCCC2)cc2cnc(Nc3ccc(N4CCNCC4)cc3)nc2n1Cc1cnoc1C(F)(F)F ; CCOC#Cc1cc2cnc(Nc3ccc(C4CCCNC4)cc3)nc2n(Cc2cncn2CCOC)c1=O ; CC(C)C#Cc1cc2cnc(Nc3ccc(C4CCN(C)C4)cc3)nc2n(C(C)C)c1=O]

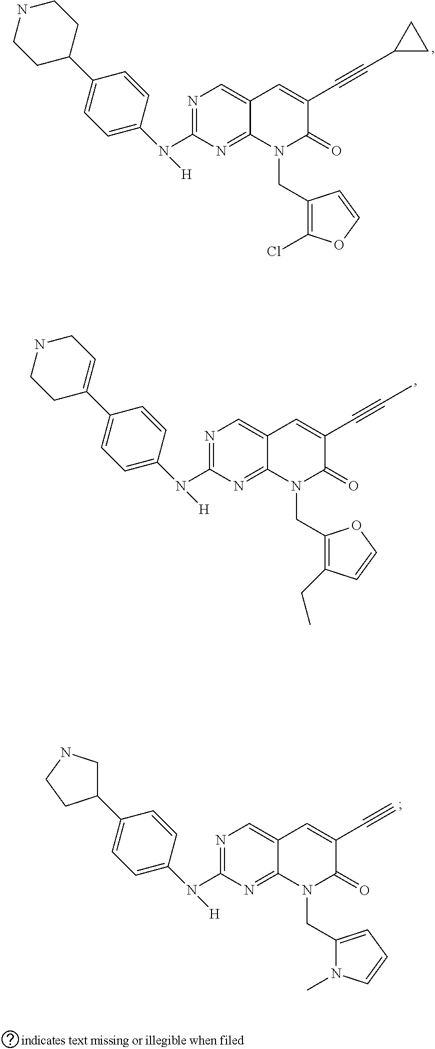 [SMILES: O=c1c(C#CC2CC2)cc2cnc(Nc3ccc(C4CCNCC4)cc3)nc2n1Cc1ccoc1Cl ; CC#Cc1cc2cnc(Nc3ccc(C4=CCNCC4)cc3)nc2n(Cc2occc2CC)c1=O ; C#Cc1cc2cnc(Nc3ccc(C4CCNC4)cc3)nc2n(Cc2cccn2C)c1=O]

or a pharmaceutically acceptable salt, solvate or N-oxide thereof.

In another embodiment are PAK inhibitors suitable for treating cancer having the structure:

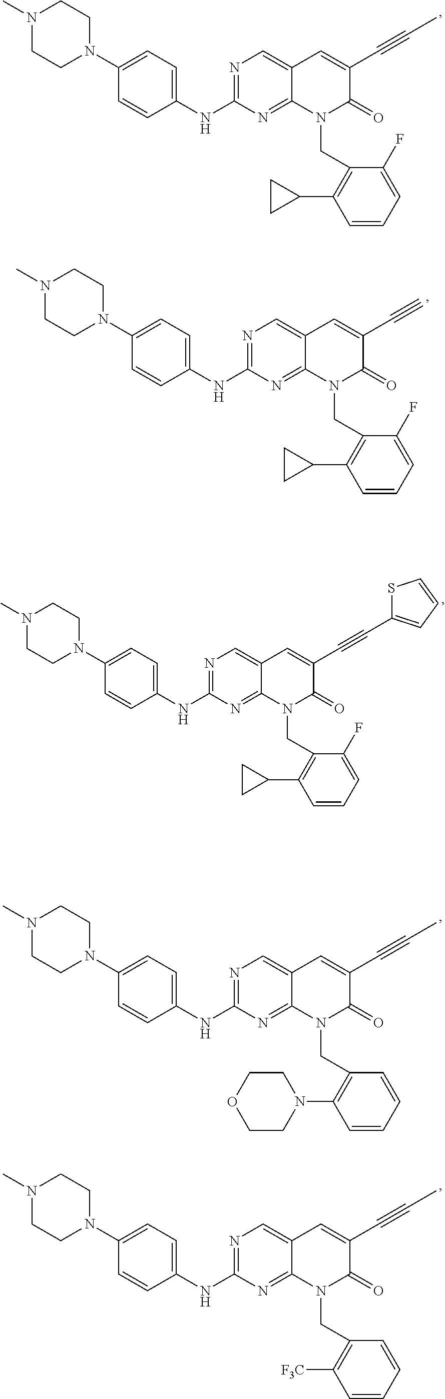

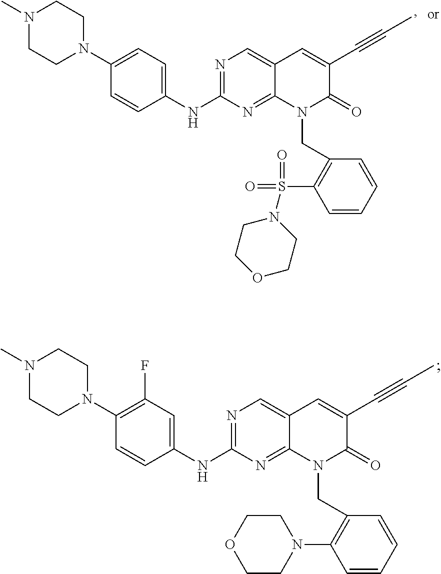

or a pharmaceutically acceptable salt, solvate or N-oxide thereof.

In some embodiments, a PAK inhibitor is a small molecule. As referred to herein, a “small molecule” is an organic molecule that is less than about 5 kilodaltons (kDa) in size. In some embodiments, the small molecule is less than about 4 kDa, 3 kDa, about 2 kDa, or about 1 kDa. In some embodiments, the small molecule is less than about 800 daltons (Da), about 600 Da, about 500 Da, about 400 Da, about 300 Da, about 200 Da, or about 100 Da. In some embodiments, a small molecule is less than about 4000 g/mol, less than about 3000 g/mol, 2000 g/mol, less than about 1500 g/mol, less than about 1000 g/mol, less than about 800 g/mol, or less than about 500 g/mol. In some embodiments, small molecules are non-polymeric. Typically, small molecules are not proteins, polypeptides, polynucleotides, oligonucleotides, polysaccharides, glycoproteins, or proteoglycans, but includes peptides of up to about 40 amino acids. A derivative of a small molecule refers to a molecule that shares the same structural core as the original small molecule, but which is prepared by a series of chemical reactions from the original small molecule. As one example, a pro-drug of a small molecule is a derivative of that small molecule. An analog of a small molecule refers to a molecule that shares the same or similar structural core as the original small molecule, and which is synthesized by a similar or related route, or art-recognized variation, as the original small molecule.

In certain embodiments, compounds described herein have one or more chiral centers. As such, all stereoisomers are envisioned herein. In various embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieve in any suitable manner, including by way of non-limiting example, by resolution of the racemic form by recrystallization techniques, by synthesis from optically-active starting materials, by chiral synthesis, or by chromatographic separation using a chiral stationary phase. In some embodiments, mixtures of one or more isomer is utilized as the therapeutic compound described herein. In certain embodiments, compounds described herein contains one or more chiral centers. These compounds are prepared by any means, including enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, chromatography, and the like.

In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenenesulfonate, mesylate and the like. Furthermore, pharmaceutically acceptable salts include, by way of non-limiting example, alkaline earth metal salts (e.g., calcium or magnesium), alkali metal salts (e.g., sodium-dependent or potassium), ammonium salts and the like.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶CI, ¹⁸F, ¹²³I, ¹²⁵I, ¹³N, ¹⁵N, ¹⁵O, ¹⁷O, ¹⁸O, ³²P, ³⁵S or the like. In some embodiments, isotopically-labeled compounds are useful in drug and/or substrate tissue distribution studies. In some embodiments, substitution with heavier isotopes such as deuterium affords certain therapeutic advantages resulting from greater metabolic stability (for example, increased in vivo half-life or reduced dosage requirements). In some embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference for such disclosure). General methods for the preparation of compound as described herein are modified by the use of appropriate reagents and conditions, for the introduction of the various moieties found in the formula as provided herein. As a guide the following synthetic methods are utilized.

Compounds described herein are synthesized using any suitable procedures starting from compounds that are available from commercial sources, or are prepared using procedures described herein.

Formation of Covalent Linkages by Reaction of an Electrophile with a Nucleophile

The compounds described herein are modified using various electrophiles and/or nucleophiles to form new functional groups or substituents. Table A entitled “Examples of Covalent Linkages and Precursors Thereof” lists selected non-limiting examples of covalent linkages and precursor functional groups which yield the covalent linkages. Table A is used as guidance toward the variety of electrophiles and nucleophiles combinations available that provide covalent linkages. Precursor functional groups are shown as electrophilic groups and nucleophilic groups.

TABLE A
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

In the reactions described, it is necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. In some embodiments it is contemplated that each protective group be removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In some embodiments carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a Pd⁰-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups are selected from:

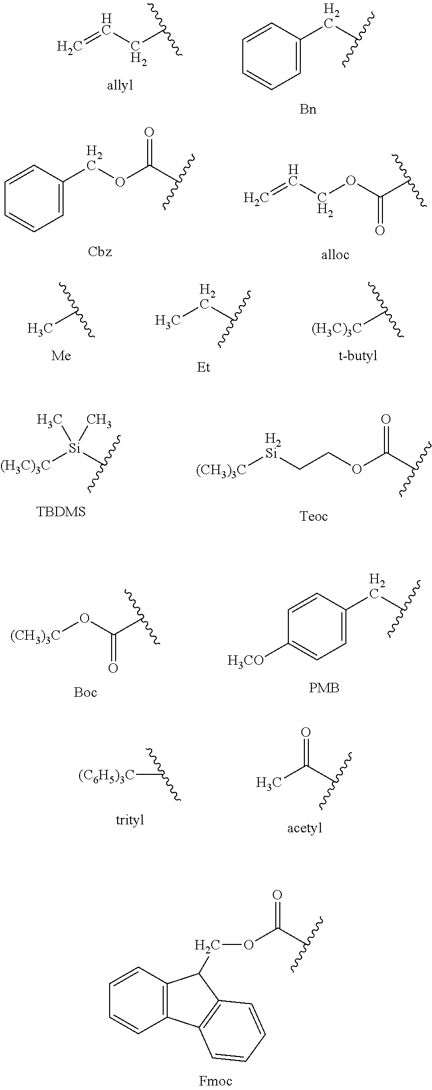

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Certain Definitions

As used herein the term “Treatment”, “treat”, or “treating” includes achieving a therapeutic benefit and/or a prophylactic benefit. Therapeutic benefit is meant to include eradication or amelioration of the underlying disorder or condition being treated. For example, in an individual with breast cancer, therapeutic benefit includes alleviation or partial and/or complete halting of the progression of the disease, or partial or complete reversal of the disease. Also, a therapeutic benefit is achieved with the eradication or amelioration of one or more of the physiological or psychological symptoms associated with the underlying condition such that an improvement is observed in the patient, notwithstanding the fact that the patient is still affected by the condition. For example, in an individual suffering from lung cancer, therapeutic benefit includes alleviation or partial and/or complete halting of coughing or wheezing, or reduction in frequency of coughing or wheezing. A prophylactic benefit of treatment includes prevention of a condition, retarding the progress of a condition, or decreasing the likelihood of occurrence of a condition. As used herein, “treat”, “treating” or “treatment” includes prophylaxis.

As used herein, the phrase “biologically active” refers to a characteristic of any substance that has activity in a biological system and/or organism. For instance, a substance that, when administered to an organism, has a biological effect on that organism is considered to be biologically active. In particular embodiments, where a protein or polypeptide is biologically active, a portion of that protein or polypeptide that shares at least one biological activity of the protein or polypeptide is typically referred to as a “biologically active” portion.

As used herein, the term “effective amount” is an amount, which when administered systemically, is sufficient to effect beneficial or desired results, such as beneficial or desired clinical results, or decrease in tumor size, alleviation of symptoms, or other desired effects. An effective amount is also an amount that produces a prophylactic effect, e.g., an amount that delays, reduces, or eliminates the appearance of a pathological or undesired condition associated with a cell proliferative disorder. An effective amount is optionally administered in one or more administrations. In terms of treatment, an “effective amount” of a composition described herein is an amount that is sufficient to palliate, alleviate, ameliorate, stabilize, reverse or slow the progression of a cell proliferative disorder e.g., tumor development, metastasis or the like. An “effective amount” includes any PAK inhibitor used alone or in conjunction with one or more agents used to treat a disease or disorder. An “effective amount” of a therapeutic agent as described herein will be determined by a patient's attending physician or other medical care provider. Factors which influence what a therapeutically effective amount will be include, the absorption profile (e.g., its rate of uptake into the blood system) of the PAK inhibitor, time elapsed since the initiation of disease, and the age, physical condition, existence of other disease states, and nutritional status of an individual being treated. Additionally, other medication the patient is receiving, e.g., anti-cancer drugs used in combination with a PAK inhibitor, will typically affect the determination of the therapeutically effective amount of the therapeutic agent to be administered.

As used herein, the term “inhibitor” refers to a molecule which is capable of inhibiting (including partially inhibiting or allosteric inhibition) one or more of the biological activities of a target molecule, e.g., a p21-activated kinase. Inhibitors, for example, act by reducing or suppressing the activity of a target molecule and/or reducing or suppressing signal transduction. In some embodiments, a PAK inhibitor described herein causes substantially complete inhibition of one or more PAKs. In some embodiments, the phrase “partial inhibitor” refers to a molecule which can induce a partial response for example, by partially reducing or suppressing the activity of a target molecule and/or partially reducing or suppressing signal transduction. In some instances, a partial inhibitor mimics the spatial arrangement, electronic properties, or some other physicochemical and/or biological property of the inhibitor. In some instances, in the presence of elevated levels of an inhibitor, a partial inhibitor competes with the inhibitor for occupancy of the target molecule and provides a reduction in efficacy, relative to the inhibitor alone. In some embodiments, a PAK inhibitor described herein is a partial inhibitor of one or more PAKs. In some embodiments, a PAK inhibitor described herein is an allosteric modulator of PAK. In some embodiments, a PAK inhibitor described herein blocks the p21 binding domain of PAK. In some embodiments, a PAK inhibitor described herein blocks the ATP binding site of PAK. In some embodiments, a PAK inhibitor is a “Type II” kinase inhibitor. In some embodiment a PAK inhibitor stabilizes PAK in its inactive conformation. In some embodiments, a PAK inhibitor stabilizes the “DFG-out” conformation of PAK.

In some embodiments, PAK inhibitors reduce, abolish, and/or remove the binding between PAK and at least one of its natural binding partners (e.g., Cdc42 or Rac). In some instances, binding between PAK and at least one of its natural binding partners is stronger in the absence of a PAK inhibitor (by e.g., 90%, 80%, 70%, 60%, 50%, 40%, 30% or 20%) than in the presence of a PAK inhibitor. Alternatively or additionally, PAK inhibitors inhibit the phosphotransferase activity of PAK, e.g., by binding directly to the catalytic site or by altering the conformation of PAK such that the catalytic site becomes inaccessible to substrates. In some embodiments, PAK inhibitors inhibit the ability of PAK to phosphorylate at least one of its target substrates, e.g., LIM kinase 1 (LIMK1), myosin light chain kinase (MLCK), cortactin; or itself. PAK inhibitors include inorganic and/or organic compounds.

In some embodiments, a PAK inhibitor suitable for the methods described herein is a direct PAK inhibitor. In some embodiments, a PAK inhibitor suitable for the methods described herein is an indirect PAK inhibitor. In some embodiments, a PAK inhibitor suitable for the methods described herein decreases PAK activity relative to a basal level of PAK activity by about 1.1 fold to about 100 fold, e.g., to about 1.2 fold, 1.5 fold, 1.6 fold, 1.7 fold, 2.0 fold, 3.0 fold, 5.0 fold, 6.0 fold, 7.0 fold, 8.5 fold, 9.7 fold, 10 fold, 12 fold, 14 fold, 15 fold, 20 fold, 30 fold, 40 fold, 50 fold, 60 fold, 70 fold, 90 fold, 95 fold, or by any other amount from about 1.1 fold to about 100 fold relative to basal PAK activity. In some embodiments, the PAK inhibitor is a reversible PAK inhibitor. In other embodiments, the PAK inhibitor is an irreversible PAK inhibitor. Direct PAK inhibitors are optionally used for the manufacture of a medicament for treating a cell proliferative disorder.

In some embodiments, a PAK inhibitor used for the methods described herein has in vitro ED₅₀ for PAK activation of less than 100 μM (e.g., less than 10 μM, less than 5 μM, less than 4 μM, less than 3 μM, less than 1 μM, less than 0.8 μM, less than 0.6 μM, less than 0.5 μM, less than 0.4 μM, less than 0.3 μM, less than less than 0.2 μM, less than 0.1 μM, less than 0.08 μM, less than 0.06 μM, less than 0.05 μM, less than 0.04 μM, less than 0.03 μM, less than less than 0.02 μM, less than 0.01 μM, less than 0.0099 μM, less than 0.0098 μM, less than 0.0097 μM, less than 0.0096 μM, less than 0.0095 μM, less than 0.0094 μM, less than 0.0093 μM, less than 0.00092 μM, or less than 0.0090 μM).

In some embodiments, a PAK inhibitor used for the methods described herein has in vitro ED₅₀ for PAK activation of less than 100 μM (e.g., less than 10 μM, less than 5 μM, less than 4 μM, less than 3 μM, less than 1 μM, less than 0.8 μM, less than 0.6 μM, less than 0.5 μM, less than 0.4 μM, less than 0.3 μM, less than less than 0.2 μM, less than 0.1 μM, less than 0.08 μM, less than 0.06 μM, less than 0.05 μM, less than 0.04 μM, less than 0.03 μM, less than less than 0.02 μM, less than 0.01 μM, less than 0.0099 μM, less than 0.0098 μM, less than 0.0097 μM, less than 0.0096 μM, less than 0.0095 μM, less than 0.0094 μM, less than 0.0093 μM, less than 0.00092 μM, or less than 0.0090 μM).

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; (4) post-translational modification of a polypeptide or protein.

As used herein the term “PAK polypeptide” or “PAK protein” or “PAK” refers to a protein that belongs in the family of p21-activated serine/threonine protein kinases. These include mammalian isoforms of PAK, e.g., the Group I PAK proteins (sometimes referred to as Group A PAK proteins), including PAK1, PAK2, PAK3, as well as the Group II PAK proteins (sometimes referred to as Group B PAK proteins), including PAK-4, PAK5, and/or PAK6. Also included as PAK polypeptides or PAK proteins are lower eukaryotic isoforms, such as the yeast Step 20 (Leberter et al., 1992, EMBO J., 11:4805; incorporated herein by reference) and/or the Dictyostelium single-headed myosin I heavy chain kinases (Wu et al., 1996, J. Biol. Chem., 271:31787; incorporated herein by reference). Representative examples of PAK amino acid sequences include, but are not limited to, human PAK1 (GenBank Accession Number AAA65441), human PAK2 (GenBank Accession Number AAA65442), human PAK3 (GenBank Accession Number AAC36097), human PAK 4 (GenBank Accession Numbers NP 005875 and CAA09820), human PAK5 (GenBank Accession Numbers CAC18720 and BAA94194), human PAK6 (GenBank Accession Numbers NP 064553 and AAF82800), human PAK7 (GenBank Accession Number Q9P286), C. elegans PAK (GenBank Accession Number BAA11844), D. melanogaster PAK (GenBank Accession Number AAC47094), and rat PAK1 (GenBank Accession Number AAB95646). In some embodiments, a PAK polypeptide comprises an amino acid sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers AAA65441, AAA65442, AAC36097, NP_{—}005875, CAA09820, CAC18720, BAA94194, NP_{—}064553, AAF82800, Q9P286, BAA11844, AAC47094, and/or AAB95646. In some embodiments, a Group I PAK polypeptide comprises an amino acid sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers AAA65441, AAA65442, and/or AAC36097.

Representative examples of PAK genes encoding PAK proteins include, but are not limited to, human PAK1 (GenBank Accession Number U24152), human PAK2 (GenBank Accession Number U24153), human PAK3 (GenBank Accession Number AF068864), human PAK-4 (GenBank Accession Number AJ011855), human PAK5 (GenBank Accession Number AB040812), and human PAK6 (GenBank Accession Number AF276893). In some embodiments, a PAK gene comprises a nucleotide sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers U24152, U24153, AF068864, AJ011855, AB040812, and/or AF276893. In some embodiments, a Group I PAK gene comprises a nucleotide sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers U24152, U24153, and/or AF068864.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent homology between the two sequences is a function of the number of identical positions shared by the sequences (i.e., % identity=# of identical positions/total # of positions (e.g., overlapping positions)×100). In one embodiment the two sequences are the same length.

To determine percent homology between two sequences, the algorithm of Karlin and Altschul (1990) Proc. Natl. Acad. Sci. USA 87:2264-2268, modified as in Karlin and Altschul (1993) Proc. Natl. Acad. Sci. USA 90:5873-5877 is used. Such an algorithm is incorporated into the NBLAST and XBLAST programs of Altschul, et al. (1990) J. Mol. Biol. 215:403-410. BLAST nucleotide searches are performed with the NBLAST program, score=100, wordlength=12 to obtain nucleotide sequences homologous to a nucleic acid molecules described or disclose herein. BLAST protein searches are performed with the XBLAST program, score=50, wordlength=3. To obtain gapped alignments for comparison purposes, Gapped BLAST is utilized as described in Altschul et al. (1997) Nucleic Acids Res. 25:3389-3402. When utilizing BLAST and Gapped BLAST programs, the default parameters of the respective programs (e.g., XBLAST and NBLAST) are used. See the website of the National Center for Biotechnology Information for further details (on the world wide web at ncbi.nlm.nih.gov). Proteins suitable for use in the methods described herein also includes proteins having between 1 to 15 amino acid changes, e.g., 1, 2, 3, 4, 5, 6, 7, 8, 9, 10, 11, 12, 13, 14, or 15 amino acid substitutions, deletions, or additions, compared to the amino acid sequence of any protein PAK inhibitor described herein. In other embodiments, the altered amino acid sequence is at least 75% identical, e.g., 77%, 80%, 82%, 85%, 88%, 90%, 92%, 95%, 97%, 98%, 99%, or 100% identical to the amino acid sequence of any protein PAK inhibitor described herein. Such sequence-variant proteins are suitable for the methods described herein as long as the altered amino acid sequence retains sufficient biological activity to be functional in the compositions and methods described herein. Where amino acid substitutions are made, the substitutions should be conservative amino acid substitutions. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff et at (1992), Proc. Natl. Acad. Sci. USA, 89:10915-10919). Accordingly, the BLOSUM62 substitution frequencies are used to define conservative amino acid substitutions that may be introduced into the amino acid sequences described or described herein. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

As used herein, the term “PAK activity,” unless otherwise specified, includes, but is not limited to, at least one of PAK protein-protein interactions, PAK phosphotransferase activity (intermolecular or intermolecular), translocation, etc of one or more PAK isoforms.

As used herein, a “PAK inhibitor” refers to any molecule, compound, or composition that directly or indirectly decreases the PAK activity. In some embodiments, PAK inhibitors inhibit, decrease, and/or abolish the level of a PAK mRNA and/or protein or the half-life of PAK mRNA and/or protein, such inhibitors are referred to as “clearance agents”. In some embodiments, a PAK inhibitor is a PAK antagonist that inhibits, decreases, and/or abolishes an activity of PAK. In some embodiments, a PAK inhibitor also disrupts, inhibits, or abolishes the interaction between PAK and its natural binding partners (e.g., a substrate for a PAK kinase, a Rac protein, a cdc42 protein, LIM kinase) or a protein that is a binding partner of PAK in a pathological condition, as measured using standard methods. In some embodiments, the PAK inhibitor is a Group I PAK inhibitor that inhibits, for example, one or more Group I PAK polypeptides, for example, PAK1, PAK2, and/or PAK3. In some embodiments, the PAK inhibitor is a PAK1 inhibitor. In some embodiments, the PAK inhibitor is a PAK2 inhibitor. In some embodiments, the PAK inhibitor is a PAK3 inhibitor. In some embodiments, the PAK inhibitor is a mixed PAK1/PAK3 inhibitor. In some embodiments, the PAK inhibitor inhibits all three Group I PAK isoforms (PAK1, PAK2 and PAK3) with equal or similar potency. In some embodiments, the PAK inhibitor is a Group II PAK inhibitor that inhibits one or more Group II PAK polypeptides, for example PAK-4, PAK5, and/or PAK6. In some embodiments, the PAK inhibitor is a PAK-4 inhibitor. In some embodiments, the PAK inhibitor is a PAK5 inhibitor. In some embodiments, the PAK inhibitor is a PAK6 inhibitor. In some embodiments, the PAK inhibitor is a PAK7 inhibitor. As used herein, a PAK5 polypeptide is substantially homologous to a PAK7 polypeptide.

In some embodiments, PAK inhibitors reduce, abolish, and/or remove the binding between PAK and at least one of its natural binding partners (e.g., Cdc42 or Rac). In some instances, binding between PAK and at least one of its natural binding partners is stronger in the absence of a PAK inhibitor (by e.g., 90%, 80%, 70%, 60%, 50%, 40%, 30% or 20%) than in the presence of a PAK inhibitor. In some embodiments, PAK inhibitors prevent, reduce, or abolish binding between PAK and a protein that abnormally accumulates or aggregates in cells or tissue in a disease state. In some instances, binding between PAK and at least one of the proteins that aggregates or accumulates in a cell or tissue is stronger in the absence of a PAK inhibitor (by e.g., 90%, 80%, 70%, 60%, 50%, 40%, 30% or 20%) than in the presence of an inhibitor.

An “individual” or an “individual,” as used herein, is a mammal. In some embodiments, an individual is an animal, for example, a rat, a mouse, a dog or a monkey. In some embodiments, an individual is a human patient. In some embodiments an “individual” or an “individual” is a human. In some embodiments, an individual suffers from a cell proliferative disorder or is suspected to be suffering from a cell proliferative disorder or is pre-disposed to a cell proliferative disorder.

In some embodiments, a pharmacological composition comprising a PAK inhibitor is “administered peripherally” or “peripherally administered.” As used herein, these terms refer to any form of administration of an agent, e.g., a therapeutic agent, to an individual that is not direct administration to the cancer or tumor, i.e., that brings the agent in contact with the non-brain side of the blood-brain barrier to treat a brain tumor. “Peripheral administration,” as used herein, includes intravenous, intra-arterial, subcutaneous, intramuscular, intraperitoneal, transdermal, by inhalation, transbuccal, intranasal, rectal, oral, parenteral, sublingual, or trans-nasal. In some embodiments, a PAK inhibitor is administered by an intracerebral route.

The terms “recurring cancer”, “recurrent cancer” or a “recurrence” are used interchangeably herein to refer to a cancer that comes back after a length of time during which it could no longer be detected following treatment. The cancer may come back in the same place as the original tumor, or it may spread to another part of the body.

The term “refractory cancer” as used herein refers to a cancer for which surgery is ineffective, which is either initially unresponsive to chemotherapy, immunotherapy, antibody therapy or radiation therapy, or which becomes unresponsive over time.

The terms “polypeptide,” and “protein” are used interchangeably herein to refer to a polymer of amino acid residues. That is, a description directed to a polypeptide applies equally to a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to compounds that have the same basic chemical structure as a naturally occurring amino acid, i.e., an α carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “isolated” and “purified” refer to a material that is substantially or essentially removed from or concentrated in its natural environment. For example, an isolated nucleic acid is one that is separated from the nucleic acids that normally flank it or other nucleic acids or components (proteins, lipids, etc.) in a sample. In another example, a polypeptide is purified if it is substantially removed from or concentrated in its natural environment. Methods for purification and isolation of nucleic acids and proteins are documented methodologies.

The term “antibody” describes an immunoglobulin whether natural or partly or wholly synthetically produced. The term also covers any polypeptide or protein having a binding domain which is, or is homologous to, an antigen-binding domain. CDR grafted antibodies are also contemplated by this term.

The term antibody as used herein will also be understood to mean one or more fragments of an antibody that retain the ability to specifically bind to an antigen, (see generally, Holliger et al., Nature Biotech. 23 (9) 1126-1129 (2005)). Non-limiting examples of such antibodies include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544 546), which consists of a VH domain; and (vi) an isolated complementarity determining region (CDR). Furthermore, although the two domains of the Fv fragment, VL and VH, are coded for by separate genes, they are optionally joined, using recombinant methods, by a synthetic linker that enables them to be made as a single protein chain in which the VL and VH regions pair to form monovalent molecules (known as single chain Fv (scFv); see e.g., Bird et al. (1988) Science 242:423 426; and Huston et al. (1988) Proc. Natl. Acad. Sci. USA 85:5879 5883; and Osbourn et al. (1998) Nat. Biotechnol. 16:778). Such single chain antibodies are also intended to be encompassed within the term antibody. Any VH and VL sequences of specific scFv is optionally linked to human immunoglobulin constant region cDNA or genomic sequences, in order to generate expression vectors encoding complete IgG molecules or other isotypes. VH and VL are also optionally used in the generation of Fab, Fv or other fragments of immunoglobulins using either protein chemistry or recombinant DNA technology. Other forms of single chain antibodies, such as diabodies are also encompassed.

“F(ab′)2” and “Fab′” moieties are optionally produced by treating immunoglobulin (monoclonal antibody) with a protease such as pepsin and papain, and includes an antibody fragment generated by digesting immunoglobulin near the disulfide bonds existing between the hinge regions in each of the two H chains. For example, papain cleaves IgG upstream of the disulfide bonds existing between the hinge regions in each of the two H chains to generate two homologous antibody fragments in which an L chain composed of VL (L chain variable region) and CL (L chain constant region), and an H chain fragment composed of VH (H chain variable region) and CHγ1 (γ1 region in the constant region of H chain) are connected at their C terminal regions through a disulfide bond. Each of these two homologous antibody fragments is called Fab′. Pepsin also cleaves IgG downstream of the disulfide bonds existing between the hinge regions in each of the two H chains to generate an antibody fragment slightly larger than the fragment in which the two above-mentioned Fab′ are connected at the hinge region. This antibody fragment is called F(ab′)2.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are documented.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “sFv” antibody fragments comprise a VH, a VL, or both a VH and VL domain of an antibody, wherein both domains are present in a single polypeptide chain. In some embodiments, the Fv polypeptide further comprises a polypeptide linker between the VH and VL domains which enables the sFv to form the desired structure for antigen binding. For a review of sFv see, e.g., Pluckthun in The Pharmacology of Monoclonal Antibodies, Vol. 113, Rosenburg and Moore eds. Springer-Verlag, New York, pp. 269 315 (1994).

A “chimeric” antibody includes an antibody derived from a combination of different mammals. The mammal is, for example, a rabbit, a mouse, a rat, a goat, or a human. The combination of different mammals includes combinations of fragments from human and mouse sources.

In some embodiments, an antibody described or described herein is a monoclonal antibody (MAb), typically a chimeric human-mouse antibody derived by humanization of a mouse monoclonal antibody. Such antibodies are obtained from, e.g., transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. In some embodiments, the transgenic mice synthesize human antibodies specific for human antigens, and the mice are used to produce human antibody-secreting hybridomas.

The term “optionally substituted” or “substituted” means that the referenced group substituted with one or more additional group(s). In certain embodiments, the one or more additional group(s) are individually and independently selected from amide, ester, alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halogen, alkoyl, alkoyloxo, isocyanato, thiocyanato, isothiocyanato, nitro, haloalkyl, haloalkoxy, fluoroalkyl, amino, alkyl-amino, dialkyl-amino, amido.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “lower alkyl” is a C₁-C₆ alkyl. A “heteroalkyl” group substitutes any one of the carbons of the alkyl group with a heteroatom having the appropriate number of hydrogen atoms attached (e.g., a CH₂ group to an NH group or an 0 group).

An “alkoxy” group refers to a (alkyl)O— group, where alkyl is as defined herein.

The term “alkylamine” refers to the —N(alkyl)ₓH_{y} group, wherein alkyl is as defined herein and x and y are selected from the group x=1, y=1 and x=2, y=0. When x=2, the alkyl groups, taken together with the nitrogen to which they are attached, optionally form a cyclic ring system.

An “amide” is a chemical moiety with formula C(O)NHR or NHC(O)R, where R is selected from alkyl, cycloalkyl, aryl, heteroaryl (bonded through a ring carbon) and heteroalicyclic (bonded through a ring carbon).

The term “ester” refers to a chemical moiety with formula —C(═O)OR, where R is selected from the group consisting of alkyl, cycloalkyl, aryl, heteroaryl and heteroalicyclic.

As used herein, the term “aryl” refers to an aromatic ring wherein each of the atoms forming the ring is a carbon atom. Aryl rings described herein include rings having five, six, seven, eight, nine, or more than nine carbon atoms. Aryl groups are optionally substituted. Examples of aryl groups include, but are not limited to phenyl, and naphthalenyl.

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

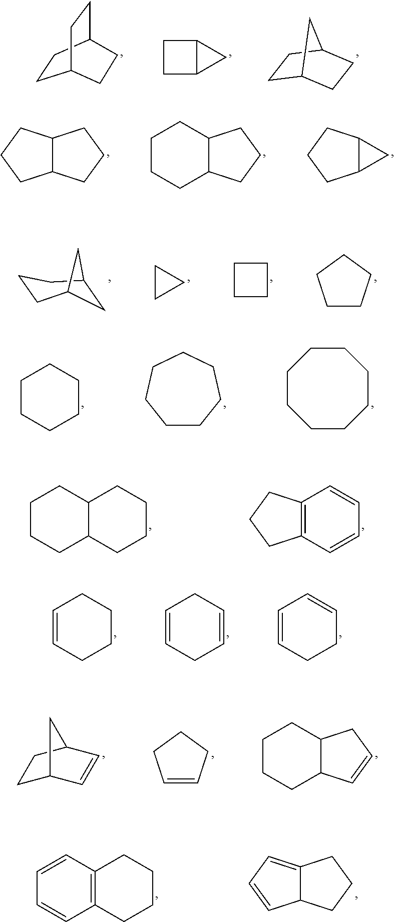

and the like. Monocyclic cycloalkyls include, but are not limited to, cyclopropyl, cyclobutyl, cyclopentyl, cyclohexyl, cycloheptyl, and cyclooctyl. Dicylclic cycloalkyls include, but are not limited to tetrahydronaphthyl, indanyl, tetrahydropentalene or the like. Polycyclic cycloalkyls include adamantane, norbornane or the like. The term cycloalkyl includes “unsaturated nonaromatic carbocyclyl” or “nonaromatic unsaturated carbocyclyl” groups both of which refer to a nonaromatic carbocycle, as defined herein, that contains at least one carbon carbon double bond or one carbon carbon triple bond.

The term “heterocyclo” refers to heteroaromatic and heteroalicyclic groups containing one to four ring heteroatoms each selected from O, S and N. In certain instances, each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are optionally substituted. In certain embodiments, heteroaryl groups are monocyclic or polycyclic. Examples of monocyclic heteroaryl groups include and are not limited to:

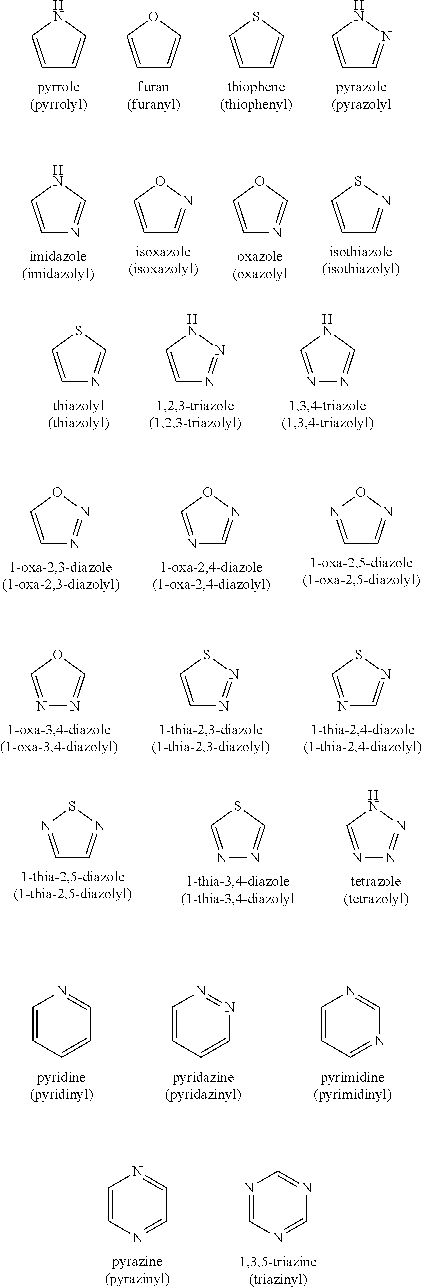

Examples of bicyclic heteroaryl groups include and are not limited to:

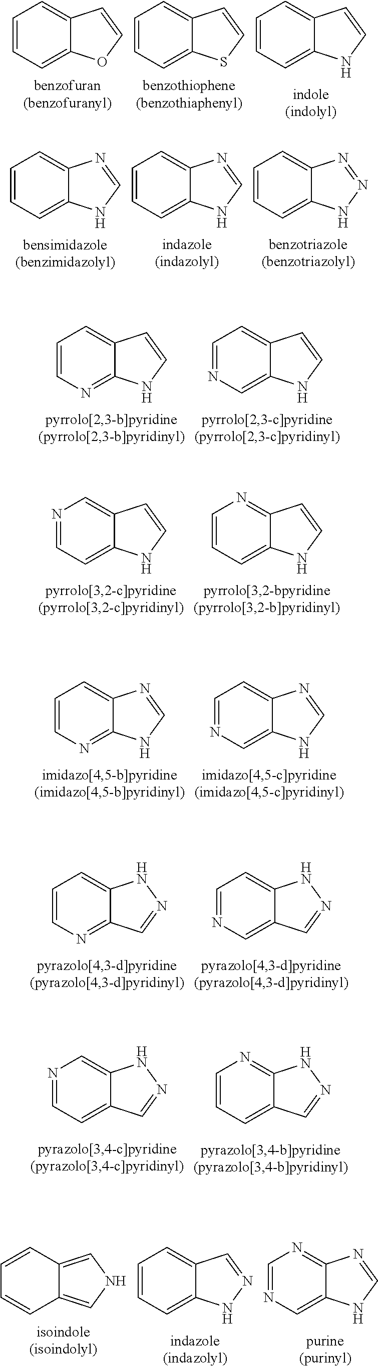

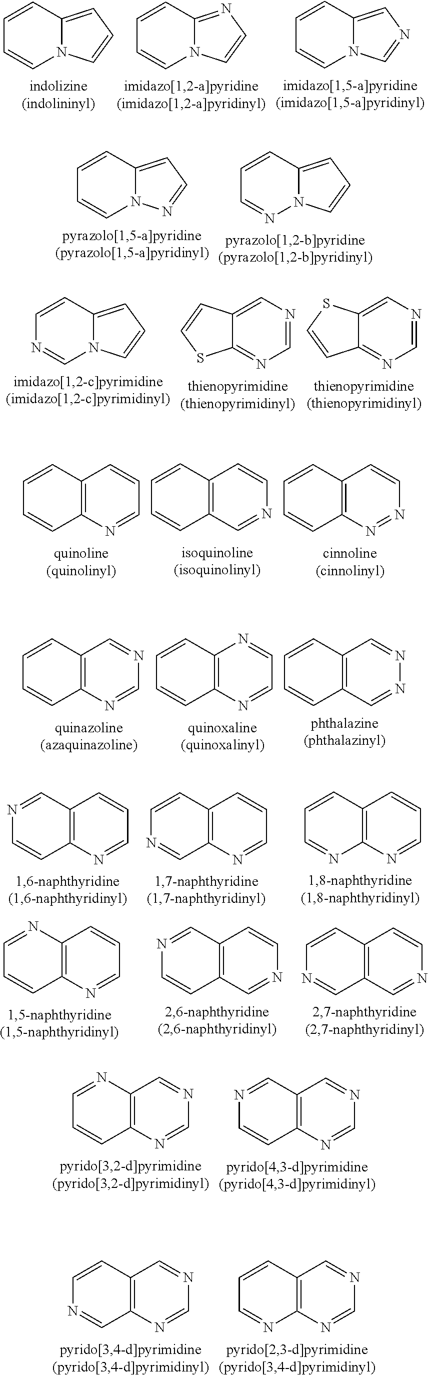

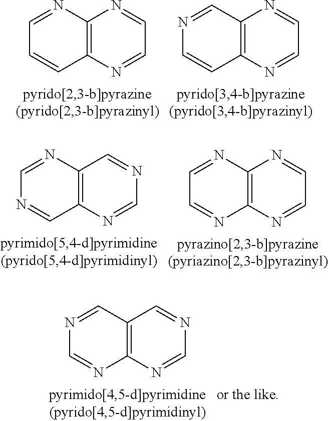

A “heteroalicyclic” group or “heterocycloalkyl” group refers to a cycloalkyl group, wherein at least one skeletal ring atom is a heteroatom selected from nitrogen, oxygen and sulfur. In some embodiments, the radicals are fused with an aryl or heteroaryl. Example of saturated heterocycloalkyl groups include

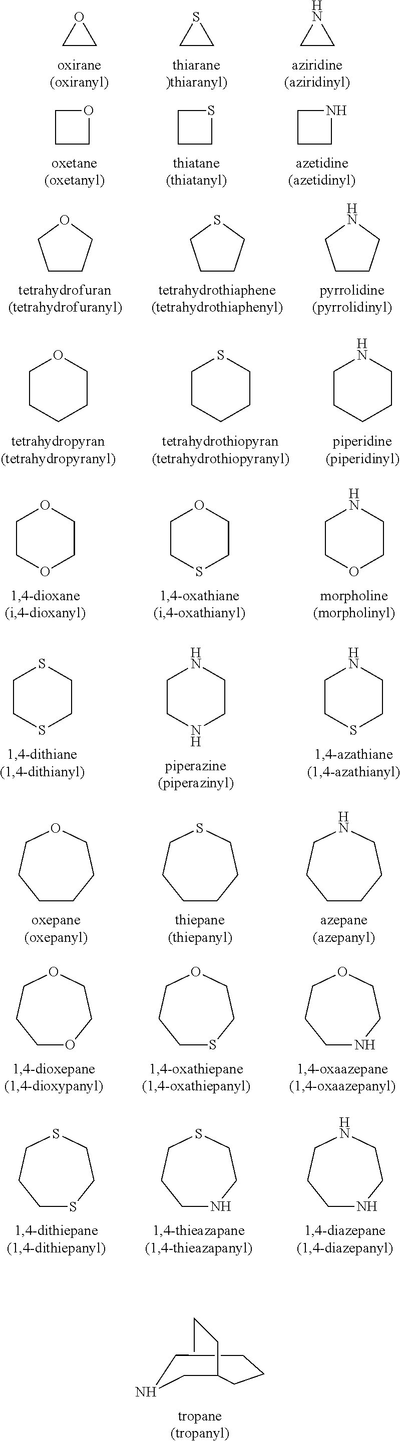

Examples of partially unsaturated heterocycloalkyl groups include

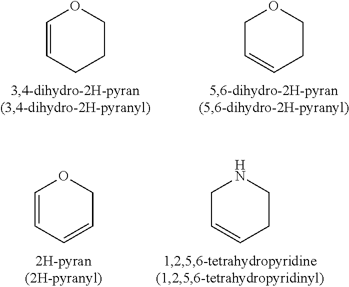 [SMILES: C1=COCCC1 ; C1=CCOCC1 ; C1=CCOC=C1 ; C1=CCNCC1]

Other illustrative examples of heterocycloalkyl groups, also referred to as non-aromatic heterocycles, include:

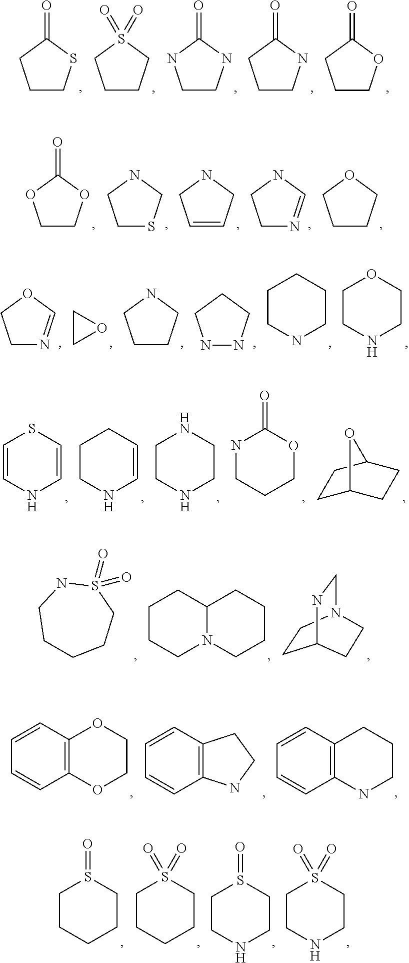

or the like.

The term heteroalicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl,” and “haloalkoxy” include alkyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃.

A “cyano” group refers to a CN group.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

### Methods

Also described herein are methods for treating cancer in a subject comprising administering to a subject in need thereof a therapeutically effective amount of a compound of Formula I-VI. As used herein, “cancer” includes any malignant growth or tumor caused by abnormal and uncontrolled cell division. “Cancer” also includes solid tumors and non-solid tumors. Examples of cancers include pancreatic cancer, gastrointestinal stromal tumors, lung cancer, stomach cancer, brain cancer, kidney cancer, breast cancer, head and neck cancer, myeloma, leukemia, lymphoma, adenocarcinoma, melanoma, cancer of the CNS, or the like.

In one embodiment is a method for treating cancer in a subject comprising administering to a subject in need thereof a therapeutically effective amount of a compound of Formula I-VI wherein the cancer is selected from ovarian, breast, colon, brain, neurofibromatosis, CML, renal cell carcinoma, gastric, leukemia, NSCLC, CNS, melanoma, prostate, T-cell lymphoma, heptocellular, bladder and glioblastoma. In one embodiment, the breast cancer is tamoxifen-resistant or intolerant breast cancer. In another embodiment, the CML is imatinib resistant or intolerant CML.

In one embodiment, is a method for modulating a p21 activated kinase comprising contacting a compound of Formula I-VI with a p21 activated kinase such that PAK expression or activation has been altered. PAK kinases have been identified as key regulators of cancer-cell signaling networks where they regulate essential biological processes. These processes include cytoskeletal dynamics, energy homeostasis, cell survival, differentiation, anchorage-independent growth, mitosis, and hormone dependence. Dysregulation of these processes by alterations in PAK expression or activation have been reported in numerous human cancers. See, e.g., Kumar R, Gururaj A E, Barnes C J, p21-activated kinases in cancer, Nat Rev Cancer, 2006; 6: 459-471, which is incorporated by reference herein to the extent it is relevant.

In another embodiment is a method for treating cancer in a subject comprising administering to a subject in need thereof a therapeutically effective amount of a compound of Formula I-VI wherein the cancer is selected from pancreatic cancer, gastrointestinal stromal tumors, lung cancer, stomach cancer, brain cancer, kidney cancer, breast cancer, head and neck cancer, myeloma, leukemia, lymphoma, adenocarcinoma, bone cancer, cutaneous or intraocular melanoma, uterine cancer, ovarian cancer, rectal cancer, cancer of the anal region, stomach cancer, colon cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, lymphocytic lymphomas, cancer of the bladder, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

In certain embodiments, a compound or a composition comprising a compound described herein is administered for prophylactic and/or therapeutic treatments. In therapeutic applications, the compositions are administered to an individual already suffering from a disease or condition, in an amount sufficient to cure or at least partially arrest the symptoms of the disease or condition. In various instances, amounts effective for this use depend on the severity and course of the disease or condition, previous therapy, an individual's health status, weight, and response to the drugs, and the judgment of the treating physician.

In some embodiments, a composition containing a therapeutically effective amount of a PAK inhibitor is administered prophylactically to an individual that while not overtly manifesting symptoms of a cell proliferative disorder has been identified as having a high risk of developing a cell proliferative disorder, e.g., an individual is identified as being a carrier of a mutation or polymorphism associated with a higher risk to develop a cell proliferative disorder (see, e.g., Banerjee and Kaye (2011), Curr Oncol Rep, 13(6)-442-9), or an individual that is from a family that has a high incidence of a cell proliferative disorder. In prophylactic applications, compounds or compositions containing compounds described herein are administered to an individual susceptible to or otherwise at risk of a particular disease, disorder or condition. In certain embodiments of this use, the precise amounts of compound administered depend on an individual's state of health, weight, and the like. Furthermore, in some instances, when a compound or composition described herein is administered to an individual, effective amounts for this use depend on the severity and course of the disease, disorder or condition, previous therapy, an individual's health status and response to the drugs, and the judgment of the treating physician.

In certain instances, wherein following administration of a selected dose of a compound or composition described herein, an individual's condition does not improve, upon the doctor's discretion the administration of a compound or composition described herein is optionally administered chronically, that is, for an extended period of time, including throughout the duration of an individual's life in order to ameliorate or otherwise control or limit the symptoms of an individual's disorder, disease or condition.

In certain embodiments, an effective amount of a given agent varies depending upon one or more of a number of factors such as the particular compound, disease or condition and its severity, the identity (e.g., weight) of an individual or host in need of treatment, and is determined according to the particular circumstances surrounding the case, including, e.g., the specific agent being administered, the route of administration, the condition being treated, and an individual or host being treated. In some embodiments, doses administered include those up to the maximum tolerable dose. In certain embodiments, about 0.02 to about 5000 mg per day, from about 1 to about 1500 mg per day, about 1 to about 100 mg/day, about 1 to about 50 mg/day, or about 1 to about 30 mg/day, or about 5 to about 25 mg/day of a compound described herein is administered. In various embodiments, the desired dose is conveniently be presented in a single dose or in divided doses administered simultaneously (or over a short period of time) or at appropriate intervals, for example as two, three, four or more sub-doses per day.

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of an individual, the severity of the disease or condition being treated, and the judgment of the practitioner.

Toxicity and therapeutic efficacy of such therapeutic regimens are optionally determined by pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. In certain embodiments, data obtained from cell culture assays and animal studies are used in formulating a range of dosage for use in human. In specific embodiments, the dosage of compounds described herein lies within a range of circulating concentrations that include the ED ₅₀ with minimal toxicity. The dosage optionally varies within this range depending upon the dosage form employed and the route of administration utilized.

### Combination Therapy

In some embodiments, one or more PAK inhibitors are used in combination with one or more other therapeutic agents to treat an individual suffering from a cell proliferative disorder. The combination of PAK inhibitors with a second therapeutic agent (e.g., anti-cancer agent, or the like) allows a reduced dose of both agents to be used thereby reducing the likelihood of side effects associated with higher dose monotherapies. In one embodiment, the dose of a second active agent is reduced in the combination therapy by at least 50% relative to the corresponding monotherapy dose, whereas the PAK inhibitor dose is not reduced relative to the monotherapy dose; in further embodiments, the reduction in dose of a second active agent is at least 75%; in yet a further embodiment, the reduction in dose of a second active agent is at least 90%. In some embodiments, the second therapeutic agent is administered at the same dose as a monotherapy dose, and the addition of a PAK inhibitor to the treatment regimen alleviates symptoms of a cell proliferative disorder that are not treated by monotherapy with the second therapeutic agent. In some embodiments, the combination of a PAK inhibitor and a second therapeutic agent is synergistic (e.g., the effect of the combination is better than the effect of each agent alone). In some embodiments, the combination of a PAK inhibitor and a second therapeutic agent is additive (e.g., the effect of the combination of active agents is about the same as the effect of each agent alone). In some embodiments, an additive effect is due to the PAK inhibitor and the second therapeutic agent modulating the same regulatory pathway. In some embodiments, an additive effect is due to the PAK inhibitor and the second therapeutic agent modulating different regulatory pathways. In some embodiments, an additive effect is due to the PAK inhibitor and the second therapeutic agent treating different symptom groups of the cell proliferative disorder (e.g., a PAK inhibitor treats negative symptoms and the second therapeutic agent treats positive symptoms of ovarian cancer). In some embodiments, administration of a second therapeutic agent treats the remainder of the same or different symptoms or groups of symptoms that are not treated by administration of a PAK inhibitor alone.

In some embodiments, administration of a combination of a PAK inhibitor and a second therapeutic agent alleviates side effects that are caused by the second therapeutic agent (e.g., side effects caused by an anti-cancer agent). In some embodiments, administration of the second therapeutic agent inhibits metabolism of an administered PAK inhibitor (e.g., the second therapeutic agent blocks a liver enzyme that degrades the PAK inhibitor) thereby increasing efficacy of a PAK inhibitor. In some embodiments, administration of a combination of a PAK inhibitor and a second therapeutic agent (e.g. a second agent that modulates cell proliferation improves the therapeutic index of a PAK inhibitor.

### Upstream Regulators of p21 Activated Kinases

In certain embodiments, compounds of Formula I-VI are optionally administered in combination with an indirect PAK modulator (e.g., an indirect PAK inhibitor) that affects the activity of a molecule that acts in a signaling pathway upstream of PAK (upstream regulators of PAK). Upstream effectors of PAK include, but are not limited to: TrkB receptors; NMDA receptors; EphB receptors; adenosine receptors; estrogen receptors; integrins; FMRP; Rho-family GTPases, including Cdc42, Rac (including but not limited to Rac1 and Rac2), CDK5, PI3 kinases, NCK, PDK1, EKT, GRB2, Chp, TC10, Tcl, and Wrch-1; guanine nucleotide exchange factors (“GEFs”), such as but not limited to GEFT, members of the Dbl family of GEFs, p21-activated kinase interacting exchange factor (PIX), DEF6, Zizimin 1, Vav1, Vav2, Dbs, members of the DOCK180 family, Kalirin-7, and Tiam1; G protein-coupled receptor kinase-interacting protein 1 (GIT1), CIB1, filamin A, Etk/Bmx, and sphingosine.

Modulators of NMDA receptor include, but are not limited to, 1-aminoadamantane, dextromethorphan, dextrorphan, ibogaine, ketamine, nitrous oxide, phencyclidine, riluzole, tiletamine, memantine, neramexane, dizocilpine, aptiganel, remacimide, 7-chlorokynurenate, DCKA (5,7-dichlorokynurenic acid), kynurenic acid, 1-aminocyclopropanecarboxylic acid (ACPC), AP7 (2-amino-7-phosphonoheptanoic acid), APV (R-2-amino-5-phosphonopentanoate), CPPene (3-[(R)-2-carboxypiperazin-4-yl]-prop-2-enyl-1-phosphonic acid); (+)-(1S,2S)-1-(4-hydroxy-phenyl)-2-(4-hydroxy-4-phenylpiperidino)-1-pro-panol; (1S,2S)-1-(4-hydro xy-3-methoxyphenyl)-2-(4-hydroxy-4-phenylpiperi-dino)-1-propanol; (3R,4S)-3-(4-(4-fluorophenyl)-4-hydroxypiperidin-1-yl-)-chroman-4,7-diol; (1R*, 2R*)-1-(4-hydroxy-3-methylphenyl)-2-(4-(4-fluoro-phenyl)-4-hydroxypiperidin-1-yl)-propan-1-ol-mesylate; and/or combinations thereof.

Modulators of estrogen receptors include, and are not limited to, PPT (4,4′,4″-(4-Propyl-[1H]-pyrazole-1,3,5-triyl)trisphenol); SKF-82958 (6-chloro-7,8-dihydroxy-3-allyl-1-phenyl-2,3,4,5-tetrahydro-1H-3-benzazepine); estrogen; estradiol; estradiol derivatives, including but not limited to 17-β estradiol, estrone, estriol, ERβ-131, phytoestrogen, MK 101 (bioNovo); VG-1010 (bioNovo); DPN (diarylpropiolitrile); ERB-041; WAY-202196; WAY-214156; genistein; estrogen; estradiol; estradiol derivatives, including but not limited to 17-13 estradiol, estrone, estriol, benzopyrans and triazolo-tetrahydrofluorenones, disclosed in U.S. Pat. No. 7,279,499, and Parker et al., Bioorg. & Med. Chem. Ltrs. 16: 4652-4656 (2006), each of which is incorporated herein by reference for such disclosure.

Modulators of TrkB include by way of example, neutorophic factors including BDNF and GDNF. Modulators of EphB include XL647 (Exelixis), EphB modulator compounds described in WO/2006081418 and US Appl. Pub. No. 20080300245, incorporated herein by reference for such disclosure, or the like.

Modulators of integrins include by way of example, ATN-161, PF-04605412, MEDI-522, Volociximab, natalizumab, Volociximab, Ro 27-2771, Ro 27-2441, etaracizumab, CNTO-95, JSM6427, cilengitide, R411 (Roche), EMD 121974, integrin antagonist compounds described in J. Med. Chem., 2002, 45 (16), pp 3451-3457, incorporated herein by reference for such disclosure, or the like.

Adenosine receptor modulators include, by way of example, theophylline, 8-Cyclopentyl-1,3-dimethylxanthine (CPX), 8-Cyclopentyl-1,3-dipropylxanthine (DPCPX), 8-Phenyl-1,3-dipropylxanthine, PSB 36, istradefylline, SCH-58261, SCH-442,416, ZM-241,385, CVT-6883, MRS-1706, MRS-1754, PSB-603, PSB-0788, PSB-1115, MRS-1191, MRS-1220, MRS-1334, MRS-1523, MRS-3777, MRE3008F20, PSB-10, PSB-11, VUF-5574, N6-Cyclopentyladenosine, CCPA, 2′-MeCCPA, GR 79236, SDZ WAG 99, ATL-146e, CGS-21680, Regadenoson, 5′-N-ethylcarboxamidoadenosine, BAY 60-6583, LUF-5835, LUF-5845, 2-(1-Hexynyl)-N-methyladenosine, CF-101 (IB-MECA), 2-C1-IB-MECA, CP-532,903, MRS-3558, Rosuvastatin, KW-3902, SLV320, mefloquine, regadenoson, or the like.

In some embodiments, compounds reducing PAK levels decrease PAK transcription or translation or reduce RNA or protein levels. In some embodiments, a compound that decreases PAK levels is an upstream effector of PAK. In some embodiments, exogenous expression of the activated forms of the Rho family GTPases Chp and cdc42 in cells leads to increased activation of PAK while at the same time increasing turnover of the PAK protein, significantly lowering its level in the cell (Hubsman et al. (2007) Biochem. J. 404: 487-497). PAK clearance agents include agents that increase expression of one or more Rho family GTPases and/or one or more guanine nucleotide exchange factors (GEFs) that regulate the activity of Rho family GTPases, in which overexpression of a Rho family GTPase and/or a GEF results in lower levels of PAK protein in cells. PAK clearance agents also include agonists of Rho family GTPases, as well as agonists of GTP exchange factors that activate Rho family GTPases, such as but not limited to agonists of GEFs of the Dbl family that activate Rho family GTPases.

Overexpression of a Rho family GTPase is optionally by means of introducing a nucleic acid expression construct into the cells or by administering a compound that induces transcription of the endogenous gene encoding the GTPase. In some embodiments, the Rho family GTPase is Rac (e.g., Rac1, Rac2, or Rac3), cdc42, Chp, TC10, Tcl, or Wrnch-1. For example, a Rho family GTPase includes Rac1, Rac2, Rac3, or cdc42. A gene introduced into cells that encodes a Rho family GTPase optionally encodes a mutant form of the gene, for example, a more active form (for example, a constitutively active form, Hubsman et al. (2007) Biochem. J. 404: 487-497). In some embodiments, a PAK clearance agent is, for example, a nucleic acid encoding a Rho family GTPase, in which the Rho family GTPase is expressed from a constitutive or inducible promoter. PAK levels in some embodiments are reduced by a compound that directly or indirectly enhances expression of an endogenous gene encoding a Rho family GTPase.

In some embodiments, compounds of Formula I-VI are optionally administered in combination with a PAK clearance agent.

In some embodiments, compounds of Formula I-VI are optionally administered in combination with a compound that directly or indirectly decreases the activation or activity of the upstream effectors of PAK. For example, in some embodiments a compound that inhibits the GTPase activity of the small Rho-family GTPases such as Rac and cdc42 thereby reduce the activation of PAK kinase. In some embodiments, the compound that decreases PAK activation is by secramine that inhibits cdc42 activation, binding to membranes and GTP in the cell (Pelish et al. (2005) Nat. Chem. Biol. 2: 39-46). In some embodiments, PAK activation is decreased by EHT 1864, a small molecule that inhibits Rac1, Rac1b, Rac2 and Rac3 function by preventing binding to guanine nucleotide association and engagement with downstream effectors (Shutes et al. (2007) J. Biol. Chem. 49: 35666-35678). In some embodiments, PAK activation is also decreased by the NSC23766 small molecule that binds directly to Rac1 and prevents its activation by Rac-specific RhoGEFs (Gao et al. (2004) Proc. Natl. Acad. Sci. U.S.A. 101: 7618-7623). In some embodiments, PAK activation is also decreased by the 16 kDa fragment of prolactin (16k PRL), generated from the cleavage of the 23 kDa prolactin hormone by matrix metalloproteases and cathepsin D in various tissues and cell types. 16k PRL down-regulates the Ras-Tiam1-Rac1-Pak1 signaling pathway by reducing Rac1 activation in response to cell stimuli such as wounding (Lee et al. (2007) Cancer Res 67:11045-11053). In some embodiments, PAK activation is decreased by inhibition of NMDA and/or AMPA receptors. Examples of modulators of AMPA receptors include and are not limited to ketamine, MK801, CNQX (6-cyano-7-nitroquinoxaline-2,3-dione); NBQX (2,3-dihydroxy-6-nitro-7-sulfamoyl-benzo[f]quinoxaline-2,3-dione); DNQX (6,7-dinitroquinoxaline-2,3-dione); kynurenic acid; 2,3-dihydroxy-6-nitro-7-sulfamoylbenzo-[f]quinoxaline; PCP or the like. In some embodiments, PAK activation is decreased by inhibition of TrkB activation. In some embodiments, PAK activation is decreased by inhibition of BDNF activation of TrkB. In some embodiments, compounds of Formula I-VI are optionally administered in combination with an antibody to BDNF. In some embodiments, PAK activation is decreased by inhibition of TrkB receptors; NMDA receptors; EphB receptors; adenosine receptors; estrogen receptors; integrins; Rho-family GTPases, including Cdc42, Rac (including but not limited to Rac1 and Rac2), CDK5, PI3 kinases, NCK, PDK1, EKT, GRB2, Chp, TC10, Tcl, and Wrch-1; guanine nucleotide exchange factors (“GEFs”), such as but not limited to GEFT, members of the Dbl family of GEFs, p21-activated kinase interacting exchange factor (PIX), DEF6, Zizimin 1, Vav1, Vav2, Dbs, members of the DOCK180 family, Kalirin-7, and Tiam1; G protein-coupled receptor kinase-interacting protein 1 (GIT1), CIB1, filamin A, Etk/Bmx, and/or binding to FMRP and/or sphingosine.

In some embodiments, compounds of Formula I-VI are optionally administered in combination with a compound that decreases PAK levels in the cell, e.g., a compound that directly or indirectly increases the activity of a guanine exchange factor (GEF) that promotes the active state of a Rho family GTPase, such as an agonist of a GEF that activates a Rho family GTPase, such as but not limited to, Rac or Cdc42. Activation of GEFs is also effected by compounds that activate TrkB, NMDA, or EphB receptors.

In some embodiments, a PAK clearance agent is a nucleic acid encoding a GEF that activates a Rho family GTPase, in which the GEF is expressed from a constitutive or inducible promoter. In some embodiments, a guanine nucleotide exchange factor (GEF), such as but not limited to a GEF that activates a Rho family GTPase is overexpressed in cells to increase the activation level of one or more Rho family GTPases and thereby lower the level of PAK in cells. GEFs include, for example, members of the Dbl family of GTPases, such as but not limited to, GEFT, PIX (e.g., alphaPIX, betaPIX), DEF6, Zizimin 1, Vav1, Vav2, Dbs, members of the DOCK180 family, hPEM-2, F1100018, kalirin, Tiam1, STEF, DOCK2, DOCK6, DOCK7, DOCKS, Asf, EhGEF3, or GEF-1. In some embodiments, PAK levels are also reduced by a compound that directly or indirectly enhances expression of an endogenous gene encoding a GEF. A GEF expressed from a nucleic acid construct introduced into cells is in some embodiments a mutant GEF, for example a mutant having enhanced activity with respect to wild type.

The clearance agent is optionally a bacterial toxin such as Salmonella typhinmurium toxin SpoE that acts as a GEF to promote cdc42 nucleotide exchange (Buchwald et al. (2002) EMBO J. 21: 3286-3295; Schlumberger et al. (2003) J. Biological Chem. 278: 27149-27159). Toxins such as SopE, fragments thereof, or peptides or polypeptides having an amino acid sequence at least 80% to 100%, e.g., 85%, 90%, 92%, 93%, 95%, 96%, 97%, 98%, 99%, or any other percent from about 80% to about 100% identical to a sequence of at least five, at least ten, at least twenty, at least thirty, at least forty, at least fifty, at least sixty, at least seventy, at least eighty, at least ninety, or at least 100 contiguous amino acids of the toxin are also optionally used as downregulators of PAK activity. The toxin is optionally produced in cells from nucleic acid constructs introduced into cells.

### Modulators of Upstream Regulators of PAKs

In some embodiments, compounds of Formula I-VI are optionally administered in combination with a modulator of an upstream regulator of PAKs. In some embodiments, a modulator of an upstream regulator of PAKs is an indirect inhibitor of PAK. In certain instances, a modulator of an upstream regulator of PAKs is a modulator of PDK1. In some instances, a modulator of PDK1 reduces of inhibits the activity of PDK1. In some instances a PDK1 inhibitor is an antisense compound (e.g., any PDK1 inhibitor described in U.S. Pat. No. 6,124,272, which PDK1 inhibitor is incorporated herein by reference). In some instances, a PDK1 inhibitor is a compound described in e.g., U.S. Pat. Nos. 7,344,870, and 7,041,687, which PDK1 inhibitors are incorporated herein by reference. In some embodiments, an indirect inhibitor of PAK is a modulator of a PI3 kinase. In some instances a modulator of a PI3 kinase is a PI3 kinase inhibitor. In some instances, a PI3 kinase inhibitor is an antisense compound (e.g., any PI3 kinase inhibitor described in WO 2001/018023, which PI3 kinase inhibitors are incorporated herein by reference). In some instances, an inhibitor of a PI3 kinase is 3-morpholino-5-phenylnaphthalen-1(4H)-one (LY294002), or a peptide based covalent conjugate of LY294002, (e.g., SF1126, Semaphore pharmaceuticals). In certain embodiments, an indirect inhibitor of PAK is a modulator of Cdc42. In certain embodiments, a modulator of Cdc42 is an inhibitor of Cdc42. In certain embodiments, a Cdc42 inhibitor is an antisense compound (e.g., any Cdc42 inhibitor described in U.S. Pat. No. 6,410,323, which Cdc42 inhibitors are incorporated herein by reference). In some instances, an indirect inhibitor of PAK is a modulator of GRB2. In some instances, a modulator of GRB2 is an inhibitor of GRB2. In some instances a GRB2 inhibitor is a GRb2 inhibitor described in e.g., U.S. Pat. No. 7,229,960, which GRB2 inhibitor is incorporated by reference herein. In certain embodiments, an indirect inhibitor of PAK is a modulator of NCK. In certain embodiments, an indirect inhibitor of PAK is a modulator of ETK. In some instances, a modulator of ETK is an inhibitor of ETK. In some instances an ETK inhibitor is a compound e.g., α-Cyano-(3,5-di-t-butyl-4-hydroxy)thiocinnamide (AG 879).

In some embodiments, indirect PAK inhibitors act by decreasing transcription and/or translation of PAK. An indirect PAK inhibitor in some embodiments decreases transcription and/or translation of a PAK. For example, in some embodiments, modulation of PAK transcription or translation occurs through the administration of specific or non-specific inhibitors of PAK transcription or translation. In some embodiments, proteins or non-protein factors that bind the upstream region of the PAK gene or the 5′ UTR of a PAK mRNA are assayed for their affect on transcription or translation using transcription and translation assays (see, for example, Baker, et al. (2003) J. Biol. Chem. 278: 17876-17884; Jiang et al. (2006) J. Chromatography A 1133: 83-94; Novoa et al. (1997) Biochemistry 36: 7802-7809; Brandi et al. (2007) Methods Enzymol. 431: 229-267). PAK inhibitors include DNA or RNA binding proteins or factors that reduce the level of transcription or translation or modified versions thereof. In other embodiments, compounds of Formula I-VI are optionally administered in combination with an agent that is a modified form (e.g., mutant form or chemically modified form) of a protein or other compound that positively regulates transcription or translation of PAK, in which the modified form reduces transcription or translation of PAK. In yet other embodiments, a transcription or translation inhibitor is an antagonist of a protein or compound that positively regulates transcription or translation of PAK, or is an agonist of a protein that represses transcription or translation.

Regions of a gene other than those upstream of the transcriptional start site and regions of an mRNA other than the 5′ UTR (such as but not limited to regions 3′ of the gene or in the 3′ UTR of an mRNA, or regions within intron sequences of either a gene or mRNA) also include sequences to which effectors of transcription, translation, mRNA processing, mRNA transport, and mRNA stability bind. In some embodiments, compounds of Formula I-VI are optionally administered in combination with a clearance agent comprising a polypeptide having homology to an endogenous protein that affects mRNA processing, transport, or stability, or is an antagonist or agonist of one or more proteins that affect mRNA processing, transport, or turnover, such that the inhibitor reduces the expression of PAK protein by interfering with PAK mRNA transport or processing, or by reducing the half-life of PAK mRNA. A PAK clearance agent in some embodiments interferes with transport or processing of a PAK mRNA, or by reducing the half-life of a PAK mRNA.

For example, PAK clearance agents decrease RNA and/or protein half-life of a PAK isoform, for example, by directly affecting mRNA and/or protein stability. In certain embodiments, PAK clearance agents cause PAK mRNA and/or protein to be more accessible and/or susceptible to nucleases, proteases, and/or the proteasome. In some embodiments, compounds of Formula I-VI are optionally administered in combination with agents that decrease the processing of PAK mRNA thereby reducing PAK activity. For example, PAK clearance agents function at the level of pre-mRNA splicing, 5′ end formation (e.g. capping), 3′ end processing (e.g. cleavage and/or polyadenylation), nuclear export, and/or association with the translational machinery and/or ribosomes in the cytoplasm. In some embodiments, PAK clearance agents cause a decrease in the level of PAK mRNA and/or protein, the half-life of PAK mRNA and/or protein by at least about 5%, at least about 10%, at least about 20%, at least about 30%, at least about 40%, at least about 50%, at least about 60%, at least about 80%, at least about 90%, at least about 95%, or substantially 100%.

In some embodiments, the clearance agent comprises one or more RNAi or antisense oligonucleotides directed against one or more PAK isoform RNAs. In some embodiments, compounds of Formula I-VI are optionally administered in combination with agents that comprise one or more ribozymes directed against one or more PAK isoform RNAs. The design, synthesis, and use of RNAi constructs, antisense oligonucleotides, and ribozymes are found, for example, in Dykxhoorn et al. (2003) Nat. Rev. Mol. Cell. Biol. 4: 457-467; Hannon et al. (2004) Nature 431: 371-378; Sarver et al. (1990) Science 247:1222-1225; Been et al. (1986) Cell 47:207-216). In some embodiments, nucleic acid constructs that induce triple helical structures are also introduced into cells to inhibit transcription of the PAK gene (Helene (1991) Anticancer Drug Des. 6:569-584).

For example, a clearance agent is in some embodiments an RNAi molecule or a nucleic acid construct that produces an RNAi molecule. An RNAi molecule comprises a double-stranded RNA of at least about seventeen bases having a 2-3 nucleotide single-stranded overhangs on each end of the double-stranded structure, in which one strand of the double-stranded RNA is substantially complementary to the target PAK RNA molecule whose downregulation is desired. “Substantially complementary” means that one or more nucleotides within the double-stranded region are not complementary to the opposite strand nucleotide(s). Tolerance of mismatches is optionally assessed for individual RNAi structures based on their ability to downregulate the target RNA or protein. In some embodiments, RNAi is introduced into the cells as one or more short hairpin RNAs (“shRNAs”) or as one or more DNA constructs that are transcribed to produce one or more shRNAs, in which the shRNAs are processed within the cell to produce one or more RNAi molecules.

Nucleic acid constructs for the expression of siRNA, shRNA, antisense RNA, ribozymes, or nucleic acids for generating triple helical structures are optionally introduced as RNA molecules or as recombinant DNA constructs. DNA constructs for reducing gene expression are optionally designed so that the desired RNA molecules are expressed in the cell from a promoter that is transcriptionally active in mammalian cells, such as, for example, the SV40 promoter, the human cytomegalovirus immediate-early promoter (CMV promoter), or the pol III and/or pol II promoter using known methods. For some purposes, it is desirable to use viral or plasmid-based nucleic acid constructs. Viral constructs include but are not limited to retroviral constructs, lentiviral constructs, or based on a pox virus, a herpes simplex virus, an adenovirus, or an adeno-associated virus (AAV).

In other embodiments, compounds of Formula I-VI are optionally administered in combination with a polypeptide that decreases the activity of PAK. Protein and peptide inhibitors of PAK are optionally based on natural substrates of PAK, e.g., Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), cortactin, cofilin, Ras, Raf, Mek, p47(phox), BAD, caspase 3, estrogen and/or progesterone receptors, NET1, Gαz, phosphoglycerate mutase-B, RhoGDI, prolactin, p41Arc, cortactin and/or Aurora-A. In some embodiments, compounds of Formula I-VI are optionally administered in combination with an agent that is based on a sequence of PAK itself, for example, the autoinhibitory domain in the N-terminal portion of the PAK protein that binds the catalytic domain of a partner PAK molecule when the PAK molecule is in its homodimeric state (Zhao et al. (1998) Mol. Cell. Biol. 18:2153-2163; Knaus et al. (1998) J. Biol. Chem. 273: 21512-21518; Hofman et al. (2004) J. Cell Sci. 117: 4343-4354). In some embodiments, polypeptide inhibitors of PAK comprise peptide mimetics, in which the peptide has binding characteristics similar to a natural binding partner or substrate of PAK.

In some embodiments, provided herein are compounds that downregulate PAK protein level. In some embodiments, the compounds described herein activate or increase the activity of an upstream regulator or downstream target of PAK. In some embodiments, compounds described herein downregulate protein level of a PAK. In some instances compounds described herein reduce at least one of the symptoms related a cell proliferative disorder by reducing the amount of PAK in a cell. In some embodiments a compound that decreases PAK protein levels in cells also decreases the activity of PAK in the cells. In some embodiments a compound that decreases PAK protein levels does not have a substantial impact on PAK activity in cells. In some embodiments a compound that increases PAK activity in cells decreases PAK protein levels in the cells.

In some embodiments, a compound that decreases the amount of PAK protein in cells decreases transcription and/or translation of PAK or increases the turnover rate of PAK mRNA or protein by modulating the activity of an upstream effector or downstream regulator of PAK. In some embodiments, PAK expression or PAK levels are influenced by feedback regulation based on the conformation, chemical modification, binding status, or activity of PAK itself. In some embodiments, PAK expression or PAK levels are influenced by feedback regulation based on the conformation, chemical modification, binding status, or activity of molecules directly or indirectly acted on by PAK signaling pathways. As used herein “binding status” refers to any or a combination of whether PAK, an upstream regulator of PAK, or a downstream effector of PAK is in a monomeric state or in an oligomeric complex with itself, or whether it is bound to other polypeptides or molecules. For example, a downstream target of PAK, when phosphorylated by PAK, in some embodiments directly or indirectly downregulates PAK expression or decrease the half-life of PAK mRNA or protein. Downstream targets of PAK include but are not limited to: Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), Ras, Raf, Mek, p47^{phox}, BAD, caspase 3, estrogen and/or progesterone receptors, NET1, Gαz, phosphoglycerate mutase-B, RhoGDI, prolactin, p41^{Arc}, cortactin and/or Aurora-A. Downregulators of PAK levels include downstream targets of PAK or fragments thereof in a phosphorylated state and downstream targets of PAK or fragments thereof in a hyperphosphorylated state.

A fragment of a downstream target of PAK includes any fragment with an amino acid sequence at least 80% to 100%, e.g., 85%, 90%, 92%, 93%, 95%, 96%, 97%, 98%, 99%, or any other percent from about 80% to about 100% identical to a sequence of at least five, at least ten, at least twenty, at least thirty, at least forty, at least fifty, at least sixty, at least seventy, at least eighty, at least ninety, or at least 100 contiguous amino acids of the downstream regulator, in which the fragment of the downstream target of PAK is able to downregulate PAK mRNA or protein expression or increase turnover of PAK mRNA or protein. In some embodiments, the fragment of a downstream regulator of PAK comprises a sequence that includes a phosphorylation site recognized by PAK, in which the site is phosphorylated.

In some embodiments, compounds of Formula I-VI are optionally administered in combination with a compound that decreases the level of PAK including a peptide, polypeptide, or small molecule that inhibits dephosphorylation of a downstream target of PAK, such that phosphorylation of the downstream target remains at a level that leads to downregulation of PAK levels.

In some embodiments, PAK activity is reduced or inhibited via activation and/or inhibition of an upstream regulator and/or downstream target of PAK. In some embodiments, the protein expression of a PAK is downregulated. In some embodiments, the amount of PAK in a cell is decreased. In some embodiments a compound that decreases PAK protein levels in cells also decreases the activity of PAK in the cells. In some embodiments a compound that decreases PAK protein levels does not decrease PAK activity in cells. In some embodiments a compound that increases PAK activity in cells decreases PAK protein levels in the cells.

In some instances, compounds of Formula I-VI are optionally administered in combination with a polypeptide that is delivered to one or more brain regions of an individual by administration of a viral expression vector, e.g., an AAV vector, a lentiviral vector, an adenoviral vector, or a HSV vector. A number of viral vectors for delivery of therapeutic proteins are described in, e.g., U.S. Pat. Nos., 7,244,423, 6,780,409, 5,661,033. In some embodiments, the PAK inhibitor polypeptide to be expressed is under the control of an inducible promoter (e.g., a promoter containing a tet-operator). Inducible viral expression vectors include, for example, those described in U.S. Pat. No. 6,953,575. Inducible expression of a PAK inhibitor polypeptide allows for tightly controlled and reversible increases of PAK inhibitor polypeptide expression by varying the dose of an inducing agent (e.g., tetracycline) administered to an individual.

### Anti-Cancer Agents

Where the subject is suffering from or at risk of suffering from a cell proliferative disorder (e.g., cancer), the subject in some embodiments is treated with a compound of Formula I-VIII in any combination with one or more anti-cancer therapy(ies). In some embodiments, one or more of the anti-cancer therapy(ies) comprises surgery, chemotherapy, radiation therapy, photodynamic therapy, gene therapy or immunotherapy. In some embodiments, the anti-cancer therapy comprises surgery, wherein the cancer or a portion thereof is physically removal from a subject. In some instances, the disease type, stage and the individual's age and condition will determine what type of surgery may be performed. In some instances, the anti-cancer therapy comprises chemotherapy. In some instances, the chemotherapy uses drugs to kill cancer cells. In some instances, these drugs target rapidly-dividing cells and attempt to inhibit this division of cells. In some embodiments, the anti-cancer therapy comprises radiation therapy. In some instances, radiation therapy uses high-energy x-rays help to destroy cancer cells and shrink tumors. In some instances, the radiation is from outside the body from a machine (e.g., external radiation). In other instances, the radiation is from radioactive materials placed directly in or around cancer cells through thin plastic tubes (e.g., internal or implant radiation). In some embodiments, the anti-cancer therapy comprises photodynamic therapy. In some instances, photodynamic therapy destroys cancer cells by using the energy from light and may also be effective when combined with surgery. In some instances, the anti-cancer therapy comprises gene therapy. This approach allows treatment to target tumors, rather than destroying healthy cells. In some embodiments, the anti-cancer therapy comprises immunotherapy. Immunotherapy (or biological therapy) treats cancer by using the body's own immune system to fight cancer cells. Another name often applies to this therapy: biological response modifiers (BRMs).

Disclosed herein is a method for treating a tumor associated with neurofibromatosis (NF) in a subject comprising administering a PAK inhibitor to the subject. Further disclosed herein is a method for treating a tumor associated with neurofibromatosis (NF) in a subject comprising administering two or more agents to the subject, wherein at least of the agents is a PAK inhibitor. In some embodiments, the neurofibromatosis is type 1 neurofibromatosis. In some embodiments, the neurofibromatosis is type 2 neurofibromatosis. In some embodiments, the method further comprises administering an anti-cancer agent. In some embodiments, the second agent is an anti-cancer agent. In some embodiments, the anti-cancer agent is an mTOR inhibitor, VEGF inhibitor. In some embodiments, the mTOR inhibitor is rapamycin or everolimus. In some embodiments, the anti-cancer agent is selected from AZD2171, AZD6244 hydrogen sulfate, bevacizumab, PTC299, pirfenidone, sorafenib, sirolimus, imiquimod, lapatinib, nilotinib, sunitinib, sunitinib malate, AMN107, PEG-Intron, or any combination thereof. In some embodiments, the second agent is anti-neurofibromatosis agent. In some instances, the anti-neurofibromatosis agent is lovastatin. In some embodiments, the method further comprises administering a proton therapy. In other embodiments, the method further comprises administering a photodynamic therapy. In some embodiments, the photodynamic therapy comprises LS11. In some embodiments, the method further comprises administering radiation therapy.

Disclosed herein is a method for treating a mesothelioma in a subject comprising administering a PAK inhibitor to the subject. Further disclosed herein is a method for treating a mesothelioma in a subject comprising administering two or more agents to the subject, wherein at least one of the agents is a PAK inhibitor. In some embodiments, the mesothelioma is a malignant mesothelioma. In some embodiments, the method further comprises administering an anti-cancer agent. In some embodiments, the second agent is an anti-cancer agent. In some embodiments, the anti-cancer agent is selected from everolimus, cisplatin, imatinib mesylate, pemetrexed, erlotinib, bevacizumab, dasatininb, ZD1839, semaxanib, gefitinib, gemcitabine, amifostine, sodium thiosulfate, mitomycin C, vinblastine sulfate, vinorlbine tartrate, ganciclovir, raltitrexed, carboplatin, doxorubicin, onconase, vorinstat, bortezomib, pazopanib, capecitabine, vatalanib, rilotumumab, trabectedin, GC1008, zoledronic acid, PF-03446962, or any combination thereof. In some embodiments, the method further comprises administering pentostatin, cyclphosphamide and SS1P. In some embodiments, the method further comprises administering oxaliplatin and gemcitabine. In some embodiments, the method further comprises administering carboplatin. In some embodiments, the method further comprises administering valproate and doxorubicin.

Disclosed herein is a method for treating a meningioma in a subject comprising administering a PAK inhibitor to the subject. Further disclosed herein is a method for treating a meningioma in a subject comprising administering two or more agents to the subject, wherein at least one of the agents is a PAK inhibitor. In some embodiments, the meningioma is a recurrent or inoperable meningioma. In some embodiments, the meningioma is a refractory meningioma. In some embodiments, the method further comprises administering an anti-cancer agent. In some embodiments, the second agent is an anti-cancer agent. In some embodiments, the anti-cancer agent is selected from sunitinib, sunitinib malate, SOM230C, SOM230B, hydroxurea, vatalinib, verapamil, imatinib mesylate, everolimus, bevacizumab, panobinostat, erlotinib, erlotinib hydrochloride, gefitinib, pioglitazone, ifosfamide, lapatinib, entinostat, ixabepilone, topotecan hydrochloride, enzastaurin hydrochloride, sodium phenylbutyrate, temozolomide, carboplatin, talabostat mesylate, talotrexin, busulfan, semaxinib, filgrastim, pegfilgrastim, trabectedin, O6-benzylguanine, temozolomide, ABT-751, romidepsin, AZD2171, thalidomide, crizotinib, ispinesib, cilengitide or any combination thereof. In some embodiments, the method further comprises administering radiation therapy. In some embodiments, the radiation therapy comprises carbon ion radiotherapy, proton radiation, proton beam radiation therapy, or intensity-modulated radiation therapy. In some embodiments, the method further comprises stereotactic radiosurgery. In some embodiments, the method further comprises administering hydroxyurea and verapamil. In some embodiments, the method further comprises administering everolimus and bevacizumab.

Disclosed herein is a method for treating a glioma in a subject comprising administering a PAK inhibitor to the subject. Further disclosed herein is a method for treating a glioma in a subject comprising administering two or more agents to the subject, wherein at least one of the agents is a PAK inhibitor. In some embodiments, the glioma is a malignant glioma. In some embodiments, the glioma is a high grade glioma or supratentorial high-grade glioma. In some embodiments, the glioma is a diffuse intrinsic pontine glioma. In some embodiments, the glioma is a recurrent glioma. In some embodiments, the method further comprises administering an anti-cancer agent. In some embodiments, the second agent is an anti-cancer agent. In some embodiments, the anti-cancer agent is selected from temozolomide, bevacizumab, irinotecan, talasporfin sodium, erlotinib hydrochloride, cilengitide, crenolanib, naltrexone, IL13-PE38QQR, AZD6244, XL765, AZD8055, 131-I-TM-601, ANG1005, vandetanib, everolimus, valproic acid, PEG-interferon alpha-2B, 2B3-101, ritnoavir, lopinavir, carboplatin, dichloroacetate, thalomid, or any combination thereof. In some embodiments, the method further comprises radiation therapy. In some embodiments, the method further comprises administering bevacizumab, Gleevac®, everolimus, or any combination thereof. In some embodiments, the radiation therapy is selected from intensity modulated radiation therapy (IMRT), sterotactic radiosurgery (SRS), intraoperative radiation therapy (IORT), image guided radiation therapy (IGRT). In some embodiments, the method further comprises immunotherapy or targeted therapy. In some embodiments, the method further comprises vaccine therapy. In some embodiments, the vaccine therapy comprises DCVax®-L.

Disclosed herein is a method for treating a schwannoma in a subject comprising administering a PAK inhibitor to the subject. Further disclosed herein is a method for treating a schwannoma in a subject comprising administering two or more agents to the subject, wherein at least one of the agents is a PAK inhibitor. In some embodiments, the method further comprises administering an anti-cancer agent. In some embodiments, the second agent is an anti-cancer agent. In some embodiments, the anti-cancer agent is selected from bevacizumab, everolimus, RAD001, lapatinib, nilotinib, Afinitor®, pazopanib, ifosfamide, dasatinib, sorafenib, dacarbazine, erlotinib, erlotinib hydrochloride, imatinib mesylate, or any combination thereof. In some embodiments, the method further comprises administering gemcitabine and docetaxel. In some embodiments, the method further comprises radiation therapy. In some instances, the radiation therapy is selected from stereotactic radiotherapy, fractionated proton radiation. In some embodiments, the method further comprises proton therapy or surgery.

Disclosed herein is a method for treating a lung cancer in a subject comprising administering a PAK inhibitor to the subject. Further disclosed herein is a method for treating a lung cancer in a subject comprising administering two or more agents to the subject, wherein at least one of the NSCLC is an advanced NSCLC. In other embodiments, the lung cancer is SCLC. In some embodiments, the method further comprises administering an anti-cancer agent. In some embodiments, the second agent is an anti-cancer agent. In some embodiments, the anti-cancer agent is selected from cisplatin, gemcitabine, pemetrexed, docetaxel, vinorelbine, or any combination thereof. In some embodiments, the anti-cancer agent is endostar, vorinostat, nimotuzumab, carboplatin, mapatumumab, paclitaxel, BIBW2992, ISIS EIF4E, figitumumab, erlotinib, cabazitaxel-XRP6258, GRN1005, panitumumab, AMG 706, dasatinib, epirubicin, NRX194204, vandetanib, ARQ197, Lacanix™, or any combination thereof. In some embodiments, the method further comprises radiation therapy, endobronchial therapy, surgery, chemotherapy, or any combination thereof. In some embodiments, radiation therapy comprises conformational radiotherapy, proton radiotherapy, thermal ablation with external beam radiation, or any combination thereof. In some embodiments, endobronchial therapy comprises photodynamic therapy. In some embodiments, the method further comprises vaccine therapy. In some embodiments, the vaccine therapy comprises a recombinant human rEGF-P64K/montanide vaccine.

Where the subject is suffering from or at risk of suffering from a cell proliferative disorder (e.g., cancer), the subject in some embodiments is treated with a compound of Formula I-VI in any combination with one or more other anti-cancer agents. In some embodiments, one or more of the anti-cancer agents are proapoptotic agents. The proapoptotic agents include, but are not limited to, antagonists of inhibitor of apoptosis proteins (IAP) (e.g., BV6, G-416). In some embodiments, one or more of the anti-cancer agents are kinase inhibitors or receptor inhibitors (e.g, EGFR inhibitors, VEGF inhibitors, or HER2 inhibitors). Examples of kinase inhibitors include, but are not limited to, EGFR kinase inhibitors (e.g., gefitinib), BCR/Abl and/or Src kinase inhibitors (e.g., dasatinib, nilotinib), Akt inhibitors (e.g, Akt VI), MEK inhibitors (e.g, U0126), tyrosine kinase inhibitors (e.g, imatinib). Examples of EGFR, VEGF and/or HER2 inhibitors include, but are not limited to, afatinib, erlotinib, lapatinib, pegaptanib, pazopanib, sunitinib, ranibixumab, vandetanib, and ZD6474. Additional examples of anti-cancer agents that are kinase inhibitors and receptor inhibitors include, but are not limited to, trastuzumab, sorafenib, mubritinib, fostamatinib, crizotinib, and cetuximab.

In some embodiments, one or more anti-cancer agents are chemotherapeutics. Examples of chemotherapeutics include, but are not limited to, alkylating agents (e.g, altretamine, cisplatin, carboplatin, oxaliplatin), anti-metabolites, plant alkaloids and terpenoids (e.g, vinca alkaloids, vinblastine, vindesine, taxanes, podophyllotoxin), topoisomerase inhibitors (e.g, irinotecan, topotecan, amsacrine, etoposide), and cytotoxic antiobiotics (e.g., doxorubicin, valrubicin, epirubicin, bleomycin).

Additional examples of anti-cancer agents include, but are not limited to, any of the following: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is an anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and in some embodiments are useful for treating cancer in combination with the compounds described herein.

Further examples of anti-cancer agents for use in combination with a compound of Formula I-VI include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

In some embodiments, other anti-cancer agents that are employed in combination with a PAK inhibitor compound include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that in some embodiments are employed in combination with a compound of Formula I-VI include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; R.sub.11 retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that in further embodiments are employed in combination with a compound of Formula I-VI include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products useful in combination with a compound of Formula I-VI include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon β).

Examples of alkylating agents that in further embodiments are employed in combination with a compound of Formula I-VI include, but are not limited to, nitrogen mustards (e.g., mechloroethamine, cyclophosphamide, chlorambucil, melphalan, etc.), ethylenimine and methylmelamines (e.g., hexamethylmelamine, thiotepa), alkyl sulfonates (e.g., busulfan), nitrosoureas (e.g., carmustine, lomusitne, semustine, streptozocin, etc.), or triazenes (decarbazine, etc.). Examples of hormones and antagonists useful in combination with a compound of Formula I-VI include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which in other embodiments are used in combination with a compound of Formula I-VI include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin Al (also known as BTO-956 and DIME), DDE-313 (Parker Hughes Institute), Fijianolide B. Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Any combination of one or more PAK inhibitors and a second therapeutic agent is compatible with any method described herein. The PAK inhibitor compositions described herein are also optionally used in combination with other therapeutic reagents that are selected for their therapeutic value for the condition to be treated. In general, the compositions described herein and, in embodiments where combinational therapy is employed, other agents do not have to be administered in the same pharmaceutical composition, and, because of different physical and chemical characteristics, are optionally administered by different routes. The initial administration is generally made according to established protocols, and then, based upon the observed effects, the dosage, modes of administration and times of administration subsequently modified.

In certain instances, it is appropriate to administer at least one PAK inhibitor composition described herein in combination with another therapeutic agent. By way of example only, if one of the side effects experienced by a patient upon receiving one of the PAK inhibitor compositions described herein is nausea, then it is appropriate to administer an anti-nausea agent in combination with the initial therapeutic agent. Or, by way of example only, the therapeutic effectiveness of a PAK inhibitor is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by a patient is increased by administering a PAK inhibitor with another therapeutic agent (which also includes a therapeutic regimen) that also has therapeutic benefit. In any case, regardless of the disease, disorder or condition being treated, the overall benefit experienced by the patient is either simply additive of the two therapeutic agents or the patient experiences a synergistic benefit.

Therapeutically-effective dosages vary when the drugs are used in treatment combinations. Suitable methods for experimentally determining therapeutically-effective dosages of drugs and other agents include, e.g., the use of metronomic dosing, i.e., providing more frequent, lower doses in order to minimize toxic side effects. Combination treatment further includes periodic treatments that start and stop at various times to assist with the clinical management of the patient.

In any case, the multiple therapeutic agents (one of which is a PAK inhibitor described herein) are administered in any order, or even simultaneously. If simultaneously, the multiple therapeutic agents are optionally provided in a single, unified form, or in multiple forms (by way of example only, either as a single pill or as two separate pills). In some embodiments, one of the therapeutic agents is given in multiple doses, or both are given as multiple doses. If not simultaneous, the timing between the multiple doses optionally varies from more than zero weeks to less than four weeks. In addition, the combination methods, compositions and formulations are not to be limited to the use of only two agents; the use of multiple therapeutic combinations are also envisioned.

The pharmaceutical agents which make up the combination therapy disclosed herein are optionally a combined dosage form or in separate dosage forms intended for substantially simultaneous administration. The pharmaceutical agents that make up the combination therapy are also optionally administered sequentially, with either therapeutic compound being administered by a regimen calling for two-step administration. The two-step administration regimen optionally calls for sequential administration of the active agents or spaced-apart administration of the separate active agents. The time period between the multiple administration steps ranges from a few minutes to several hours, depending upon the properties of each pharmaceutical agent, such as potency, solubility, bioavailability, plasma half-life and kinetic profile of the pharmaceutical agent. Circadian variation of the target molecule concentration are optionally used to determine the optimal dose interval.

In addition, a PAK inhibitor is optionally used in combination with procedures that provide additional or synergistic benefit to the patient. By way of example only, patients are expected to find therapeutic and/or prophylactic benefit in the methods described herein, wherein pharmaceutical composition of a PAK inhibitor and/or combinations with other therapeutics are combined with genetic testing to determine whether that individual is a carrier of a mutant gene that is correlated with certain diseases or conditions.

A PAK inhibitor and the additional therapy(ies) are optionally administered before, during or after the occurrence of a disease or condition, and the timing of administering the composition containing a PAK inhibitor varies in some embodiments. Thus, for example, the PAK inhibitor is used as a prophylactic and administered continuously to individuals with a propensity to develop conditions or diseases in order to prevent the occurrence of the disease or condition. The PAK inhibitors and compositions are optionally administered to an individual during or as soon as possible after the onset of the symptoms or diagnosis. The administration of the compounds are optionally initiated within the first 48 hours of the onset of the symptoms or diagnosis, preferably within the first 48 hours of the onset of the symptoms or diagnosis, more preferably within the first 6 hours of the onset of the symptoms or diagnosis, and most preferably within 3 hours of the onset of the symptoms or diagnosis. The initial administration is optionally via any route practical, such as, for example, an intravenous injection, a bolus injection, infusion over 5 minutes to about 5 hours, a pill, a capsule, transdermal patch, buccal delivery, and the like, or combination thereof. A PAK inhibitor is optionally administered as soon as is practicable after the onset of a disease or condition is detected or suspected, and for a length of time necessary for the treatment of the disease, such as, for example, from about 1 month to about 3 months. The length of treatment optionally varies for each individual, and the length is then determined using the known criteria. For example, the PAK inhibitor or a formulation containing the PAK inhibitor is administered for at least 2 weeks, preferably about 1 month to about 5 years, and more preferably from about 1 month to about 3 years.

In some embodiments, the particular choice of compounds depends upon the diagnosis of the attending physicians and their judgment of the condition of an individual and the appropriate treatment protocol. The compounds are optionally administered concurrently (e.g., simultaneously, essentially simultaneously or within the same treatment protocol) or sequentially, depending upon the nature of the disease, disorder, or condition, the condition of an individual, and the actual choice of compounds used. In certain instances, the determination of the order of administration, and the number of repetitions of administration of each therapeutic agent during a treatment protocol, is based on an evaluation of the disease being treated and the condition of an individual.

In some embodiments, therapeutically-effective dosages vary when the drugs are used in treatment combinations. Methods for experimentally determining therapeutically-effective dosages of drugs and other agents for use in combination treatment regimens are described in the literature.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a therapeutic compound described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g, from more than zero weeks to less than four weeks. In some embodiments, the additional therapeutic agent is utilized to achieve reversal or amelioration of symptoms of a cell proliferative disorder, whereupon the therapeutic agent described herein (e.g., a compound of any one of Formula I-VI is subsequently administered). In addition, the combination methods, compositions and formulations are not to be limited to the use of only two agents; the use of multiple therapeutic combinations are also envisioned (including two or more compounds described herein).

In certain embodiments, a dosage regimen to treat, prevent, or ameliorate the condition(s) for which relief is sought, is modified in accordance with a variety of factors. These factors include the disorder from which an individual suffers, as well as the age, weight, sex, diet, and medical condition of an individual. Thus, in various embodiments, the dosage regimen actually employed varies and deviates from the dosage regimens set forth herein.

### Examples of Pharmaceutical Compositions and Methods of Administration

Provided herein, in certain embodiments, are compositions comprising a therapeutically effective amount of any compound described herein (e.g., a compound of Formula I-VI).

Pharmaceutical compositions are formulated using one or more physiologically acceptable carriers including excipients and auxiliaries which facilitate processing of the active compounds into preparations which are used pharmaceutically. Proper formulation is dependent upon the route of administration chosen. A summary of pharmaceutical compositions is found, for example, in Remington: The Science and Practice of Pharmacy, Nineteenth Ed (Ea hston, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include one or more PAK inhibitors and a pharmaceutically acceptable diluent(s), excipient(s), or carrier(s). In addition, the PAK inhibitor is optionally administered as pharmaceutical compositions in which it is mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of a PAK inhibitor with other chemical components, such as carriers, stabilizers, diluents, dispersing agents, suspending agents, thickening agents, and/or excipients. The pharmaceutical composition facilitates administration of the PAK inhibitor to an organism. In practicing the methods of treatment or use provided herein, therapeutically effective amounts of a PAK inhibitor are administered in a pharmaceutical composition to a mammal having a condition, disease, or disorder to be treated. Preferably, the mammal is a human. A therapeutically effective amount varies depending on the severity and stage of the condition, the age and relative health of an individual, the potency of the PAK inhibitor used and other factors. The PAK inhibitor is optionally used singly or in combination with one or more therapeutic agents as components of mixtures.

The pharmaceutical formulations described herein are optionally administered to an individual by multiple administration routes, including but not limited to, oral, parenteral (e.g., intravenous, subcutaneous, intramuscular), intranasal, buccal, topical, rectal, or transdermal administration routes. By way of example only, Example 13a describes a parenteral formulation, Example 13f describes a rectal formulation. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions will include at least one PAK inhibitor, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these PAK inhibitors having the same type of activity. In some situations, PAK inhibitors exist as tautomers. All tautomers are included within the scope of the compounds presented herein. Additionally, the PAK inhibitor exists in unsolvated as well as solvated forms with pharmaceutically acceptable solvents such as water, ethanol, and the like. The solvated forms of the PAK inhibitors presented herein are also considered to be disclosed herein.

“Carrier materials” include any commonly used excipients in pharmaceutics and should be selected on the basis of compatibility with compounds disclosed herein, such as, a PAK inhibitor, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

Moreover, the pharmaceutical compositions described herein, which include a PAK inhibitor, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, a formulation comprising a PAK inhibitor is a solid drug dispersion. A solid dispersion is a dispersion of one or more active ingredients in an inert carrier or matrix at solid state prepared by the melting (or fusion), solvent, or melting-solvent methods (Chiou and Riegelman, Journal of Pharmaceutical Sciences, 60, 1281 (1971)). The dispersion of one or more active agents in a solid diluent is achieved without mechanical mixing. Solid dispersions are also called solid-state dispersions. In some embodiments, any compound described herein (e.g., a compound of Formula I-VI is formulated as a spray dried dispersion (SDD). An SDD is a single phase amorphous molecular dispersion of a drug in a polymer matrix. It is a solid solution prepared by dissolving the drug and a polymer in a solvent (e.g., acetone, methanol or the like) and spray drying the solution. The solvent rapidly evaporates from droplets which rapidly solidifies the polymer and drug mixture trapping the drug in amorphous form as an amorphous molecular dispersion. In some embodiments, such amorphous dispersions are filled in capsules and/or constituted into oral powders for reconstitution. Solubility of an SDD comprising a drug is higher than the solubility of a crystalline form of a drug or a non-SDD amorphous form of a drug. In some embodiments of the methods described herein, PAK inhibitors are administered as SDDs constituted into appropriate dosage forms described herein.

Pharmaceutical preparations for oral use are optionally obtained by mixing one or more solid excipient with a PAK inhibitor, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active compound doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. By way of example, Example 13b describes a solid dosage formulation that is a capsule. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of a PAK inhibitor are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a PAK inhibitor, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

The pharmaceutical solid oral dosage forms including formulations described herein, which include a PAK inhibitor, are optionally further formulated to provide a controlled release of the PAK inhibitor. Controlled release refers to the release of the PAK inhibitor from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to an individual over an extended period of time according to a predetermined profile. Such release rates provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein, which include a PAK inhibitor, are delivered using a pulsatile dosage form. A pulsatile dosage form is capable of providing one or more immediate release pulses at predetermined time points after a controlled lag time or at specific sites. Pulsatile dosage forms including the formulations described herein, which include a PAK inhibitor, are optionally administered using a variety of pulsatile formulations that include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the PAK inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the PAK inhibitor is optionally in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of the PAK inhibitor and a suitable powder base such as lactose or starch. By way of example, Example 13e describes an inhalation formulation.

Buccal formulations that include a PAK inhibitor include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period, wherein the delivery of the PAK inhibitor, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner. By way of example, Examples 13c and 13d describe sublingual formulations.

Transdermal formulations of a PAK inhibitor are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144. By way of example, Example 13 g describes a topical formulation.

The transdermal formulations described herein include at least three components: (1) a formulation of a PAK inhibitor; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of a PAK inhibitor employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery of the PAK inhibitor is optionally accomplished by means of iontophoretic patches and the like. Additionally, transdermal patches provide controlled delivery of the PAK inhibitor. The rate of absorption is optionally slowed by using rate-controlling membranes or by trapping the PAK inhibitor within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing the PAK inhibitor optionally with carriers, optionally a rate controlling barrier to deliver the PAK inhibitor to the skin of the host at a controlled and predetermined rate over a prolonged period of time, and means to secure the device to the skin.

Formulations that include a PAK inhibitor suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, a PAK inhibitor is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of the PAK inhibitor in water soluble form. Additionally, suspensions of the PAK inhibitor are optionally prepared as appropriate oily injection suspensions.

In some embodiments, the PAK inhibitor is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

The PAK inhibitor is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

The PAK inhibitor is optionally used in the preparation of medicaments for the prophylactic and/or therapeutic treatment of a cell proliferative disorder that would benefit, at least in part, from amelioration of symptoms. In addition, a method for treating any of the diseases or conditions described herein in an individual in need of such treatment, involves administration of pharmaceutical compositions containing at least one PAK inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

In the case wherein the patient's condition does not improve, upon the doctor's discretion the administration of the PAK inhibitor is optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the PAK inhibitor is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical compositions described herein are in unit dosage forms suitable for single administration of precise dosages. In unit dosage form, the formulation is divided into unit doses containing appropriate quantities of one or more PAK inhibitor. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The daily dosages appropriate for the PAK inhibitor are from about 0.01 to about 2.5 mg/kg per body weight. An indicated daily dosage in the larger mammal, including, but not limited to, humans, is in the range from about 0.5 mg to about 1000 mg, conveniently administered in divided doses, including, but not limited to, up to four times a day or in extended release form. Suitable unit dosage forms for oral administration include from about 1 to about 500 mg active ingredient, from about 1 to about 250 mg of active ingredient, or from about 1 to about 100 mg active ingredient. The foregoing ranges are merely suggestive, as the number of variables in regard to an individual treatment regime is large, and considerable excursions from these recommended values are not uncommon. Such dosages are optionally altered depending on a number of variables, not limited to the activity of the PAK inhibitor used, the disease or condition to be treated, the mode of administration, the requirements of an individual, the severity of the disease or condition being treated, and the judgment of the practitioner.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. PAK inhibitors exhibiting high therapeutic indices are preferred. The data obtained from cell culture assays and animal studies is optionally used in formulating a range of dosage for use in human. The dosage of such PAK inhibitors lies preferably within a range of circulating concentrations that include the ED50 with minimal toxicity. The dosage optionally varies within this range depending upon the dosage form employed and the route of administration utilized.

### Assays for Identification and Characterization of PAK Inhibitors

Small molecule PAK inhibitors are optionally identified in high-throughput in vitro or cellular assays as described in, e.g., Yu et at (2001), J Biochem (Tokyo); 129(2):243-251; Rininsland et at (2005), BMC Biotechnol, 5:16; and Allen et at (2006), ACS Chem Biol; 1(6):371-376. PAK inhibitors suitable for the methods described herein are available from a variety of sources including both natural (e.g., plant extracts) and synthetic. For example, candidate PAK inhibitors are isolated from a combinatorial library, i.e., a collection of diverse chemical compounds generated by either chemical synthesis or biological synthesis by combining a number of chemical “building blocks.” For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks, as desired. Theoretically, the systematic, combinatorial mixing of 100 interchangeable chemical building blocks results in the synthesis of 100 million tetrameric compounds or 10 billion pentameric compounds. See Gallop et al. (1994), J. Med. Chem. 37(9), 1233. Each member of a library may be singular and/or may be part of a mixture (e.g. a “compressed library”). The library may comprise purified compounds and/or may be “dirty” (i.e., containing a quantity of impurities). Preparation and screening of combinatorial chemical libraries are documented methodologies. See Cabilly, ed., Methods in Molecular Biology, Humana Press, Totowa, N.J., (1998). Combinatorial chemical libraries include, but are not limited to: diversomers such as hydantoins, benzodiazepines, and dipeptides, as described in, e.g., Hobbs et al. (1993), Proc. Natl. Acad. Sci. U.S.A. 90, 6909; analogous organic syntheses of small compound libraries, as described in Chen et al. (1994), J. Amer. Chem. Soc., 116: 2661; Oligocarbamates, as described in Cho, et al. (1993), Science 261, 1303; peptidyl phosphonates, as described in Campbell et al. (1994), J. Org. Chem., 59: 658; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514). In addition, numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.). Devices for the preparation of combinatorial libraries are commercially available (see, e.g., 357 MPS, 390 MPS from Advanced Chem Tech, Louisville, Ky.; Symphony from Rainin, Woburn, Mass.; 433A from Applied Biosystems, Foster City, Calif.; and 9050 Plus from Millipore, Bedford, Mass.). A number of robotic systems have also been developed for solution phase chemistries. These systems include automated workstations like the automated synthesis apparatus developed by Takeda Chemical Industries, LTD (Osaka, Japan), and many robotic systems utilizing robotic arms (Zymate II). Any of the above devices are optionally used to generate combinatorial libraries for identification and characterization of PAK inhibitors which mimic the manual synthetic operations performed by small molecule PAK inhibitors suitable for the methods described herein. Any of the above devices are optionally used to identify and characterize small molecule PAK inhibitors suitable for the methods disclosed herein. In many of the embodiments disclosed herein, PAK inhibitors, PAK binding molecules, and PAK clearance agents are disclosed as polypeptides or proteins (where polypeptides comprise two or more amino acids). In these embodiments, the inventors also contemplate that PAK inhibitors, binding molecules, and clearance agents also include peptide mimetics based on the polypeptides, in which the peptide mimetics interact with PAK or its upstream or downstream regulators by replicating the binding or substrate interaction properties of PAK or its regulators. Nucleic acid aptamers are also contemplated as PAK inhibitors, binding molecules, and clearance agents, as are small molecules other than peptides or nucleic acids. For example, in some embodiments small molecule PAK binding partners, inhibitors, or clearance agents, or small molecule agonists or antagonists of PAK modulators or targets, are designed or selected based on analysis of the structure of PAK or its modulators or targets and binding interactions with interacting molecules, using “rational drug design” (see, for example Jacobsen et al. (2004) Molecular Interventions 4:337-347; Shi et al. (2007) Bioorg. Med. Chem. Lett. 17:6744-6749).

The identification of potential PAK inhibitors is determined by, for example, assaying the in vitro kinase activity of PAK in the presence of candidate inhibitors. In such assays, PAK and/or a characteristic PAK fragment produced by recombinant means is contacted with a substrate in the presence of a phosphate donor (e.g., ATP) containing radiolabeled phosphate, and PAK-dependent incorporation is measured. “Substrate” includes any substance containing a suitable hydroxyl moiety that can accept the γ-phosphate group from a donor molecule such as ATP in a reaction catalyzed by PAK. The substrate may be an endogenous substrate of PAK, i.e. a naturally occurring substance that is phosphorylated in unmodified cells by naturally-occurring PAK or any other substance that is not normally phosphorylated by PAK in physiological conditions, but may be phosphorylated in the employed conditions. The substrate may be a protein or a peptide, and the phosphylation reaction may occur on a serine and/or threonine residue of the substrate. For example, specific substrates, which are commonly employed in such assays include, but are not limited to, histone proteins and myelin basic protein. In some embodiments, PAK inhibitors are identified using IMAP® technology.

Detection of PAK dependent phosphorylation of a substrate can be quantified by a number of means other than measurement of radiolabeled phosphate incorporation. For example, incorporation of phosphate groups may affect physiochemical properties of the substrate such as electrophoretic mobility, chromatographic properties, light absorbance, fluorescence, and phosphorescence. Alternatively, monoclonal or polyclonal antibodies can be generated which selectively recognize phosphorylated forms of the substrate from non-phosphorylated forms whereby allowing antibodies to function as an indicator of PAK kinase activity.

High-throughput PAK kinase assays can be performed in, for example, microtiter plates with each well containing PAK kinase or an active fragment thereof, substrate covalently linked to each well, P³² radiolabled ATP and a potential PAK inhibitor candidate. Microtiter plates can contain 96 wells or 1536 wells for large scale screening of combinatorial library compounds. After the phosphorylation reaction has completed, the plates are washed leaving the bound substrate. The plates are then detected for phosphate group incorporation via autoradiography or antibody detection. Candidate PAK inhibitors are identified by their ability to decease the amount of PAK phosphotransferase ability upon a substrate in comparison with PAK phosphotransferase ability alone.

The identification of potential PAK inhibitors may also be determined, for example, via in vitro competitive binding assays on the catalytic sites of PAK such as the ATP binding site and/or the substrate binding site. For binding assays on the ATP binding site, a known protein kinase inhibitor with high affinity to the ATP binding site is used such as staurosporine. Staurosporine is immobilized and may be fluorescently labeled, radiolabeled or in any manner that allows detection. The labeled staurosporine is introduced to recombinantly expressed PAK protein or a fragment thereof along with potential PAK inhibitor candidates. The candidate is tested for its ability to compete, in a concentration-dependant manner, with the immobilized staurosporine for binding to the PAK protein. The amount of staurosporine bound PAK is inversely proportional to the affinity of the candidate inhibitor for PAK. Potential inhibitors would decrease the quantifiable binding of staurosporine to PAK. See e.g., Fabian et al (2005) Nat. Biotech., 23:329. Candidates identified from this competitive binding assay for the ATP binding site for PAK would then be further screened for selectivity against other kinases for PAK specificity.

The identification of potential PAK inhibitors may also be determined, for example, by in cyto assays of PAK activity in the presence of the inhibitor candidate. Various cell lines and tissues may be used, including cells specifically engineered for this purpose. In cyto screening of inhibitor candidates may assay PAK activity by monitoring the downstream effects of PAK activity. Such effects include, but are not limited to, the formation of peripheral actin microspikes and or associated loss of stress fibers as well as other cellular responses such as growth, growth arrest, differentiation, or apoptosis. See e.g., Zhao et al., (1998) Mol. Cell. Biol. 18:2153. For example in a PAK yeast assay, yeast cells grow normally in glucose medium. Upon exposure to galactose however, intracellular PAK expression is induced, and in turn, the yeast cells die. Candidate compounds that inhibit PAK activity are identified by their ability to prevent the yeast cells from dying from PAK activation.

Alternatively, PAK-mediated phosphorylation of a downstream target of PAK can be observed in cell based assays by first treating various cell lines or tissues with PAK inhibitor candidates followed by lysis of the cells and detection of PAK mediated events. Cell lines used in this experiment may include cells specifically engineered for this purpose. PAK mediated events include, but are not limited to, PAK mediated phosphorylation of downstream PAK mediators. For example, phosphorylation of downstream PAK mediators can be detected using antibodies that specifically recognize the phosphorylated PAK mediator but not the unphosphorylated form. These antibodies have been described in the literature and have been extensively used in kinase screening campaigns. In some instances a phospho LIMK antibody is used after treatment of HeLa cells stimulated with EGF or sphingosine to detect downstream PAK signaling events.

The identification of potential PAK inhibitors may also be determined, for example, by in vivo assays involving the use of animal models, including transgenic animals that have been engineered to have specific defects or carry markers that can be used to measure the ability of a candidate substance to reach and/or affect different cells within the organism. For example, transgenic mice with aberrant c-myc expression have a high propensity to develop lymphoma (Harris et al., 1988, J Exp Med, 167(2):353-71). Thus, identification of PAK inhibitors can comprise administering a candidate to a c-myc transgenic mice and observing for decreased lymphoma development as a readout for PAK inhibition.

Administration of the candidate to the animal is via any clinical or non-clinical route, including but not limited to oral, nasal, buccal and/or topical administrations. Additionally or alternatively, administration may be intratracheal instillation, bronchial instillation, intradermal, subcutaneous, intramuscular, intraperitoneal, inhalation, and/or intravenous injection.

### EXAMPLES

The following specific examples are to be construed as merely illustrative, and not limitative of the remainder of the disclosure in any way whatsoever.

From the above discussion and these Examples, one skilled in the art can ascertain the preferred features of this invention, and without departing from the spirit and scope thereof, can make various changes and modification of the invention to adapt it to various uses and conditions. See U.S. Provisional Application Ser. No. 61/352,985, U.S. Provisional Application Ser. No. 61/250,262, U.S. Provisional Application Ser. No. 61/353,054, U.S. Provisional Application Ser. No. 61/353,619, U.S. Provisional Application Ser. No. 61/353,622, U.S. Provisional Application Ser. No. 61/353,085, U.S. Provisional Application Ser. No. 61/353,624, U.S. Provisional Application Ser. No. 61/473,683, International Patent Application No. PCT/US2011/039856, International Patent Application No. PCT/US2010/052106, International Patent Application No. PCT/US2011/040075, International Patent Application No. PCT/US2011/040082, International Patent Application No. PCT/US2011/039863, and International Patent Application No. PCT/US2011/040089, which are each incorporated by reference in its entirety.

All synthetic chemistry was performed in standard laboratory glassware unless indicated otherwise in the examples. Commercial reagents were used as received.

Analytical LC/MS A was performed on an Agilent 1200 system with a variable wavelength detector and Agilent 6110 Single quadrupole mass spectrometer, alternating positive and negative ion scans. (AN/B)

Analytical LC/MS B was performed on an Agilent 1200 system with a variable wavelength detector and Agilent G1956A Single quadrupole mass spectrometer, positive or negative ion scans. (N)

Analytical LC/MS C was performed on an Agilent 1100 system with a variable wavelength detector and Agilent G1946D Single quadrupole mass spectrometer, positive or negative ion scans (AY)

Analytical LC/MS D was performed on an Agilent 1200 system with a variable wavelength detector and Agilent 6110 Single quadrupole mass spectrometer, positive or negative ion scans (AS/F)

Analytical LC/MS E was performed on an Agilent 1100 system with a variable wavelength detector and Agilent G1946A Single quadrupole mass spectrometer, positive or negative ion scans. (AX)

Analytical LC/MS F was performed on an Agilent 1100 system with a variable wavelength detector and Agilent G1946A Single quadrupole mass spectrometer, positive or negative ion scans. (I/E/W)

Analytical LC/MS G was performed on a SHIMADZU LC-20AB system with a variable wavelength detecto and SHIMADZU 2010EV Single quadrupole mass spectrometer, positive ion scans. (R)

Retention times were determined from the extracted 220 nm chromatogram. ¹H NMR was performed on a Bruker DRX-400 at 400 MHz. Microwave reactions were performed in a Biotage Initiator using the instrument software to control heating time and pressure. Silica gel chromatography was performed manually.

Preparative HPLC method A: Preparative HPLC was performed on a Waters 1525/2487 with UV detection at 220 nm and manual collection.

HPLC column: ASB-C18 21.2×150 mm.
HPLC Gradient: 25 mL/min, (0.01% HCL)water:acetonitrile; the gradient shape was optimized for individual separations.

Preparative HPLC method B:

HPLC column: Phenomenex 21.2×150 mm.
HPLC Gradient: 25 mL/min, (0.1% FA)water:acetonitrile; the gradient shape was optimized for individual separations.

### Example 1

### Synthesis of 8-(3-Cyclopropyl-thiophen-2-ylmethyl)-2-[4-(4-methyl-piperazin-1-yl)-phenylamino]-8H-pyrido[2,3-d]pyrimidin-7-one (5)

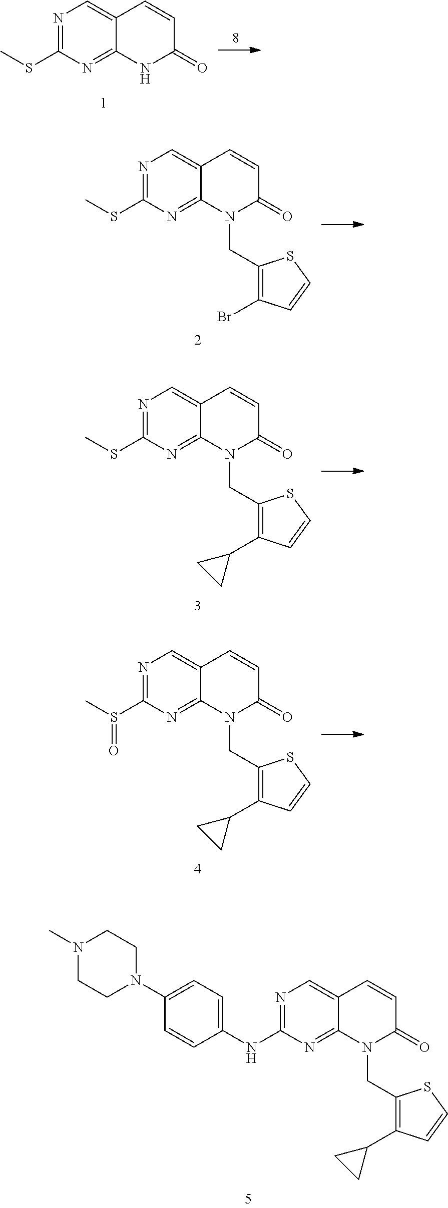 [SMILES: CSc1ncc2ccc(=O)[nH]c2n1 ; CSc1ncc2ccc(=O)n(Cc3sccc3Br)c2n1 ; CSc1ncc2ccc(=O)n(Cc3sccc3C3CC3)c2n1 ; CS(=O)c1ncc2ccc(=O)n(Cc3sccc3C3CC3)c2n1 ; CN1CCN(c2ccc(Nc3ncc4ccc(=O)n(Cc5sccc5C5CC5)c4n3)cc2)CC1]

### Preparation of Intermediate Compound

### Synthesis of intermediate 3-bromo-2-chloromethyl-thiophene (8)

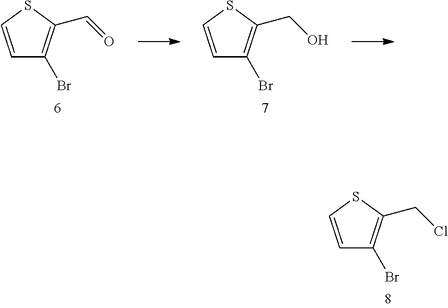 [SMILES: O=Cc1sccc1Br ; OCc1sccc1Br ; ClCc1sccc1Br]

### Step 1: Synthesis of (3-bromo-thiophen-2-yl)-methanol (7)

To a solution of 3-bromothiophene-2-carbaldehyde (6) (500 mg, 2.62 mmol) in methanol (10 mL) was added sodium borohydride (169 mg, 4.47 mmol) in small portions at 0° C. and the reaction was stirred for 2 hrs. The solvent was evaporated and the residue partitioned between ethyl acetate (20 mL) and 10% ammonium chloride solution (10 mL). The organic layer was washed with water (10 mL), dried over sodium sulfate and evaporated. The title compound (505 mg, 2.62 mmol, 100%) was obtained as a yellow oil.

### Step 2: Synthesis of 3-bromo-2-chloromethyl-thiophene (8)

(3-Bromo-thiophen-2-yl)-methanol (7) (505 mg, 2.62 mmol) was dissolved in dichloromethane (20 mL) and thionyl chloride (357 μL, 4.89 mmol) was added. The mixture was heated at reflux for 4 hrs, then evaporated. The crude product was purified by column chromatography on silica, eluting with dichloromethane. The title compound (466 mg, 2.20 mmol, 84%) was obtained as a pale yellow oil.

### Synthesis of 8-(3-Cyclopropyl-thiophen-2-ylmethyl)-2-[4-(4-methyl-piperazin-1-yl)-phenylamino]-8H-pyrido[2,3-d]pyrimidin-7-one (5)

### Step 1: Synthesis of 8-(3-bromo-thiophen-2-ylmethyl)-2-methylsulfanyl-8H-pyrido[2,3-d]-pyrimidin-7-one (2)

To a solution of 2-methylsulfanyl-8H-pyrido[2,3-d]pyrimidin-7-one (1) (300 mg, 1.55 mmol) in anhydrous dimethylformamide (10 mL) sodium hydride (60% in mineral oil, 93 mg, 2.33 mmol) was added and stirred at room temperature. After 30 min, a solution of 3-bromo-2-chloromethyl-thiophene (8) (426 mg, 2.01 mmol) in anhydrous dimethylformamide (1 mL) was added slowly and the stirring was continued for 18 h. The resulting mixture was poured into ice water (50 g) then extracted with ethyl acetate (3×30 mL). The combined organic layers was dried over sodium sulfate, filtered and evaporated. The title compound (686 mg, 1.86 mmol, quant.) was obtained as a light brown solid. ESMS m/z 368 (M+H)⁺.

### Step 2: Synthesis of 8-(3-cyclopropyl-thiophen-2-ylmethyl)-2-methylsulfanyl-8H-pyrido[2,3-d]pyrimidin-7-one (3)

8-(3-Bromo-thiophen-2-ylmethyl)-2-methylsulfanyl-8H-pyrido[2,3-d]pyrimidin-7-one (2) (350 mg, 0.95 mmol), cyclopropylboronic acid (245 mg, 2.85 mmol), K₃PO₄ (605 g, 2.85 mmol) and PdCl₂(Pcy₃)₂ (70 mg, 0.09 mmol) were mixed under argon in a degassed mixture of toluene and water (20:1, 8 mL). The resulting suspension was refluxed for 14 h, cooled and diluted with water (8 mL). The two phases were separated, the aqueous layer was washed extracted with toluene (2×10 mL), and the combined organic layers were dried over sodium sulfate, filtered and evaporated. The residue was purified by column chromatography using dichloromethane:ethyl acetate (100:3) as the eluent. The title compound (156 mg, 0.47 mmol, 49%) was obtained as a solid. ESMS m/z 330 (M+H)⁺.

### Step 3: Synthesis of 8-(3-cyclopropyl-thiophen-2-ylmethyl)-2-methanesulfinyl-8H-pyrido[2,3-d]-pyrimidin-7-one (4)

To a solution of 8-(3-cyclopropyl-thiophen-2-ylmethyl)-2-methylsulfanyl-8H-pyrido[2,3-d]pyrimidin-7-one (3) (155 mg, 0.47 mmol) in dichloromethane (5 mL) was added 3-chloroperbenzoic acid (77%, 106 mg, 0.47 mmol) in dichloromethane (5 mL) at 0-5° C. and the mixture was stirred at 0-5° C. for 1 h. The reaction mixture was washed with saturated sodium bicarbonate solution (1×10 mL) then water (1×10 mL). The organic layer was dried over sodium sulfate, filtered and evaporated to dryness. The crude compound was purified by column chromatography using dichloromethane:methanol (100:3) to yield 8-(3-cyclopropyl-thiophen-2-ylmethyl)-2-methanesulfinyl-8Hpyrido[2,3-d]pyrimidin-7-one as a white solid (118 mg, 0.34 mmol, 72%). ESMS m/z 346 (M+H)⁺.

### Step 4: Synthesis of 8-(3-Cyclopropyl-thiophen-2-ylmethyl)-2-[4-(4-methyl-piperazin-1-yl)-phenylamino]-8H-pyrido[2,3-d]pyrimidin-7-one (5)

8-(3-Cyclopropyl-thiophen-2-ylmethyl)-2-methanesulfinyl-8H-pyrido[2,3-d]pyrimidin-7-one (4) (118 mg, 0.34 mmol) and 4-(4-methylpiperazino)aniline (65 g, 0.34 mmol) were stirred at 140° C. for 6 h. The reaction mixture was purified by column chromatography using dichloromethane:methanol (9:1) and the solid product was suspended in diisopropyl ether and the solid was collected to give the title compound (52 mg, 0.11 mmol, 32%) as a light yellow solid. ESMS m/z 473 (M+H) ¹H NMR (400 MHz, CDCl₃) δ ppm 8.49 (s, 1H) 7.49 (d, J=9.3 Hz, 1H) 7.46 (d, J=8.5 Hz, 2H) 7.18 (s, 1H) 7.00 (d, J=5.3 Hz, 1H) 6.92 (d, J=8.5 Hz, 2H) 6.40-6.49 (m, 2H) 5.78 (s, 2H) 3.12-3.25 (m, 4H) 2.60 (d, J=4.5 Hz, 4H) 2.30-2.39 (m, 4H) 0.75-0.91 (m, 2H) 0.54-0.65 (m, 2H).

### Example 2

### Synthesis of 8-(5-cyclopropyl-thiazol-4-ylmethyl)-2-[4-(4-methyl-piperazin-1-yl)-phenylamino]-8H-pyrido[2,3-d]pyrimidin-7-one (11)

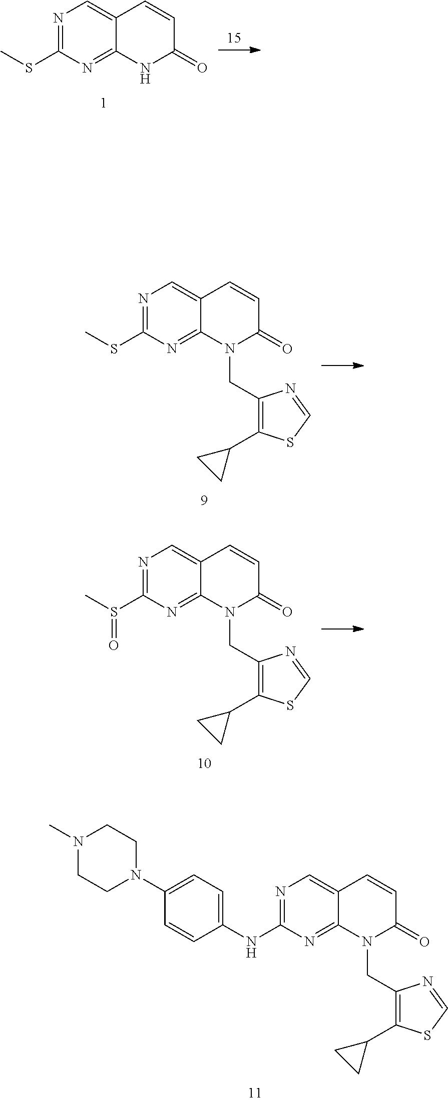 [SMILES: CSc1ncc2ccc(=O)[nH]c2n1 ; CSc1ncc2ccc(=O)n(Cc3ncsc3C3CC3)c2n1 ; CS(=O)c1ncc2ccc(=O)n(Cc3ncsc3C3CC3)c2n1 ; CN1CCN(c2ccc(Nc3ncc4ccc(=O)n(Cc5ncsc5C5CC5)c4n3)cc2)CC1]

### Preparation of Intermediate Compound

### Synthesis of intermediate 4-chloromethyl-5-cyclopropyl-thiazole (15)

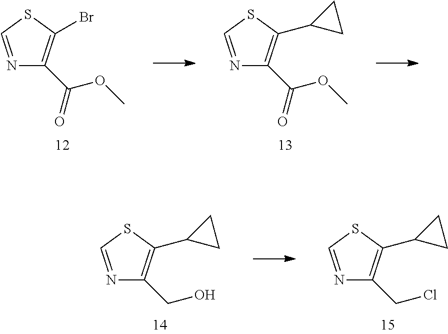

### Step 1: Synthesis of 5-cyclopropyl-thiazole-4-carboxylic acid methyl ester (13)

5-Bromothiazole-4-carboxylic acid methyl ester (12) (325 mg, 1.46 mmol), cyclopropylboronic acid (282 mg, 3.28 mmol), K₃PO₄ (697 g, 3.28 mmol) and Pd(PPh₃)₄ (110 mg, 0.09 mmol) were mixed under argon in a degassed mixture of toluene and water (20:1, 9 mL). The resulting suspension was irradiated for 2 h at 120° C. in a microwave reactor. The reaction mixture was diluted with water (8 mL), the two phases were separated, the aqueous layer was washed with dichloromethane (3×10 mL), and the combined organic layers was dried over sodium sulfate, filtered and evaporated. The residue was purified by column chromatography using n-hexane:ethyl acetate (2:1) as eluent. The title compound (137 mg, 0.75 mmol, 51%) was obtained as a yellow oil. ESMS m/z 184 (M+H)⁺.

### Step 2: Synthesis of (5-cyclopropyl-thiazol-4-yl)-methanol (14)

To a solution of 5-cyclopropyl-thiazole-4-carboxylic acid methyl ester (13) (219 mg, 1.19 mmol) in anhydrous tetrahydrofuran (5 mL) at 0° C. lithium aluminum hydride (45 mg, 1.19 mmol) was added in small portions and the mixture was stirred for 2 h. Water (5 mL) was added dropwise, then the mixture was allowed to reach room temperature. The mixture was diluted with ethyl acetate (20 mL) and brine (10 mL), the two phases were separated, and the aqueous layer was extracted with ethyl acetate (10 mL). The combined organic layers were dried over sodium sulfate, filtered and evaporated. The title compound (127 mg, 0.82 mmol, 69%) was obtained as a yellow oil. ESMS m/z 156 (M+H)⁺.

### Step 3: Synthesis of 4-chloromethyl-5-cyclopropyl-thiazole (15)

(5-Cyclopropyl-thiazol-4-yl)-methanol (14) (127 mg, 0.82 mmol) was dissolved in anhydrous dichloromethane (5 mL) and thionyl chloride (300 μL, 4.09 mmol) was added. After refluxing for 4 h the reaction mixture was evaporated. The title compound (140 mg, 0.81 mmol, 99%) was obtained as a brown oil.

### Synthesis of 8-(5-cyclopropyl-thiazol-4-ylmethyl)-2-[4-(4-(4-methyl-piperazin-1-yl)-phenylamino]-8H-pyrido[2,3-d]pyrimidin-7-one (11)

### Step 1: Synthesis of 8-(5-cyclopropyl-thiazol-4-ylmethyl)-2-methylsulfanyl-8H-pyrido[2,3-d]-pyrimidin-7-one (9)

To a solution of 2-methylsulfanyl-8H-pyrido[2,3-d]pyrimidin-7-one (1) (120 mg, 0.62 mmol) in anhydrous dimethylformamide (2 mL), sodium hydride (60% in mineral oil, 37 mg, 0.93 mmol) was added and stirred at room temperature. After 30 min, a solution of 4-chloromethyl-5-cyclopropyl-thiazole (15) (140 mg, 0.81 mmol) in anhydrous dimethylformamide (2 mL) was added slowly and the stirring was continued for 18 h. The resulting mixture was poured into ice water (20 g), and extracted with dichloromethane (3×20 mL). The combined organic layers were dried over sodium sulfate, filtered and evaporated. The residue was purified by column chromatography using dichloromethane:ethyl acetate (1:1) as eluent. The title compound (100 mg, 0.30 mmol, 48%) was obtained as a beige foam. ESMS m/z 331 (M+H)⁺.

### Step 2: Synthesis of 8-(5-cyclopropyl-thiazol-4-ylmethyl)-2-methanesulfinyl-8H-pyrido[2,3-d]pyrimidin-7-one (10)

To a solution of 8-(5-cyclopropyl-thiazol-4-ylmethyl)-2-methylsulfanyl-8H-pyrido[2,3-d]-pyrimidin-7-one (9) (100 mg, 0.30 mmol) in dichloromethane (10 mL) was added 3-chloroperbenzoic acid (77%, 61 mg, 0.27 mmol) in dichloromethane (2 mL) at 0-5° C. and the mixture was stirred at 0-5° C. for 1 h. The reaction mixture was washed with saturated sodium bicarbonate solution (1×10 mL), and the aqueous layer was back extracted with dichloromethane (15 mL). The combined organic layers were washed with water (10 mL), dried over sodium sulfate, filtered and evaporated. The crude compound was purified by column chromatography using dichloromethane:methanol (100:3) to yield the title compound as a beige solid (70 mg, 0.20 mmol, 67%). ESMS m/z 347 (M+H)⁺.

### Step 3: Synthesis of 8-(5-cyclopropyl-thiazol-4-ylmethyl)-2-[4-(4-methyl-piperazin-1-yl)-phenylamino]-8H-pyrido[2,3-d]pyrimidin-7-one (11)

8-(5-Cyclopropyl-thiazol-4-ylmethyl)-2-methanesulfinyl-8H-pyrido[2,3-d]pyrimidin-7-one (10) (70 mg, 0.20 mmol) and 4-(4-methylpiperazino)aniline (38 mg, 0.20 mmol) were stirred at 140° C. for 6 h. The reaction mixture was purified by column chromatography eluting with dichloromethane:methanol (95:5) and the solid product was suspended in diisopropyl ether and collected by filtration to give the title compound (10 mg, 0.02 mmol, 10%) as a light yellow solid. ESMS m/z 474 (M+H)⁺; ¹H NMR (400 MHz, CDCl₃) δ ppm 8.50 (s, 1H) 8.39 (s, 1H) 7.53 (d, J=9.3 Hz, 1H) 7.37 (d, J=8.8 Hz, 2H) 7.14 (s, 1H) 6.86 (d, J=8.8 Hz, 2H) 6.47 (d, J=9.3 Hz, 1H) 5.71 (s, 2H) 3.12-3.22 (m, 4H) 2.54-2.63 (m, 4H) 2.36 (s, 3H) 2.11-2.22 (m, 1H) 0.99-1.07 (m, 2H) 0.68-0.76 (m, 2H).

### Example 3

### Synthesis of 6-(2-chloro-4-(1,3,4-oxadiazol-2-yl)phenyl)-8-ethyl-2-(4-(4-methylpiperazin-1-yl)phenylamino)pyrido[2,3-d]pyrimidin-7(8H)-one (22)

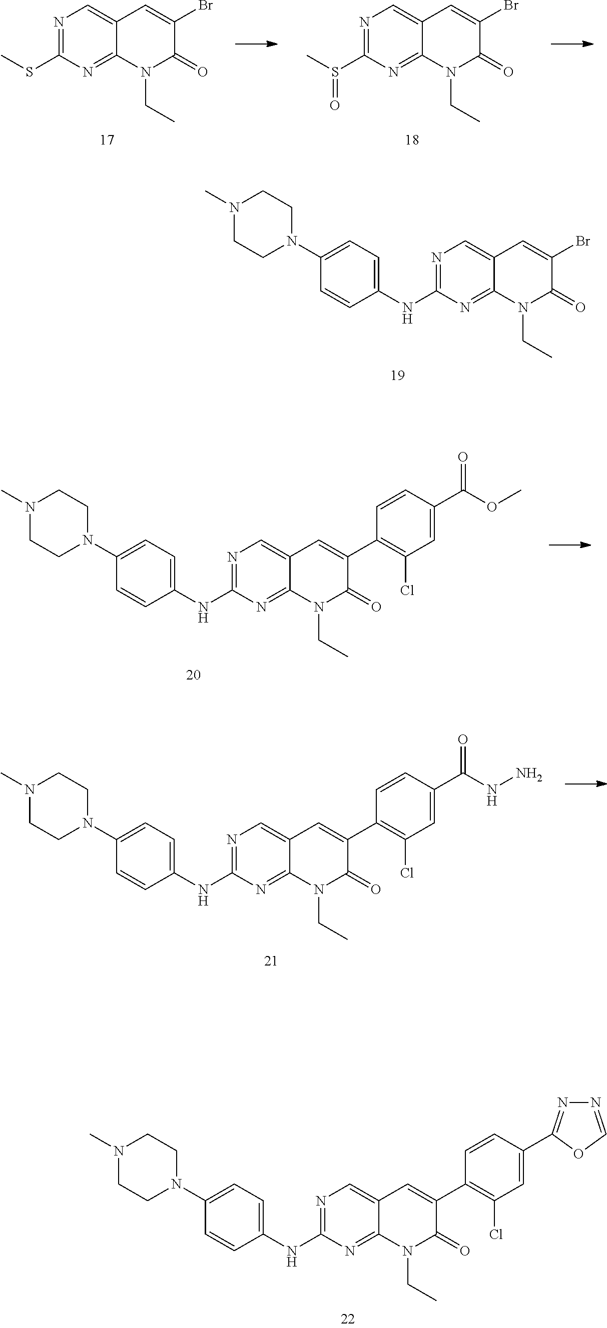

### Preparation of Intermediate Compound

### Synthesis of intermediate 6-bromo-8-ethyl-2-(methylthio)pyrido[2,3-d]pyrimidin-7(8H)-one (17)

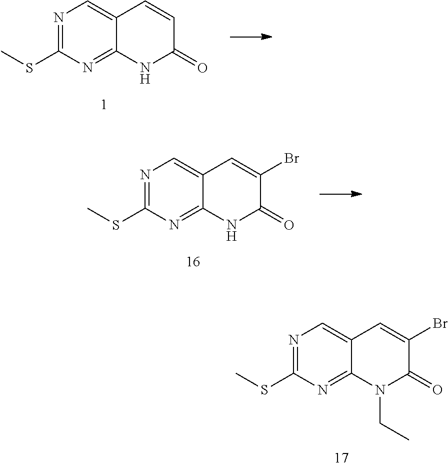 [SMILES: CSc1ncc2ccc(=O)[nH]c2n1 ; CSc1ncc2cc(Br)c(=O)[nH]c2n1 ; CCn1c(=O)c(Br)cc2cnc(SC)nc21]

### Step 1: Synthesis of 6-bromo-2-(methylthio)pyrido[2,3-d]pyrimidin-7(8H)-one (16)

To a solution of 2-methylsulfanyl-8H-pyrido[2,3-d]pyrimidin-7-one (1) (1.00 g, 5.18 mmol) in anhydrous dimethylformamide (25 mL) was added N-bromosuccinimide (0.99 g, 5.59 mmol) portionwise at room temperature, and the reaction mixture was stirred for 18 h. The mixture was concentrated, and the solid was triturated with hot water (1×20 mL), filtered, and washed with isopropanol to give title compound as a pale yellow solid (0.68 g, 2.50 mmol, 48%). ESMS m/z 272 (M+H)⁺; ¹H NMR (400 MHz, DMSO-d₆) δ ppm 12.88 (br. s., 1H), 8.84 (s, 1H), 8.47 (s, 1H), 2.57 (s, 3H).

### Step 2: Synthesis of 6-bromo-8-ethyl-2-(methylthio)pyrido[2,3-d]pyrimidin-7(8H)-one (17)

To a suspension of NaH (60%, 0.15 g, 3.75 mmol) in anhydrous dimethylformamide (10 mL) was added 6-bromo-2-(methylthio)pyrido[2,3-d]pyrimidin-7(8H)-one (16) (0.68 g, 2.50 mmol) at room temperature and the reaction was stirred at 50° C. for 0.5 h. The reaction mixture was cooled to room temperature, ethyl bromide (0.22 mL, 0.32 g, 2.93 mmol) was added, and the reaction was stirred at 50° C. for 1.5 h. The mixture was poured into ice water (10 g), and the white precipitate was collected to give 6-bromo-8-ethyl-2-(methylthio)pyrido[2,3-d]pyrimidin-7(8H)-one (3, 0.57 g, 1.90 mmol, 76%). ESMS m/z 300 (M+H)⁺. The material was used without any further purification.

### Synthesis of 6-(2-chloro-4-(1,3,4-oxadiazol-2-yl)phenyl)-8-ethyl-2-(4-(4-methylpiperazin-1-yl)phenylamino)pyrido[2,3-d]pyrimidin-7(8H)-one (22)

### Step 1: Synthesis of 6-bromo-8-ethyl-2-methanesulfinyl-8H-pyrido[2,3-d]pyrimidin-7-one (18)

To a solution of 6-bromo-8-ethyl-2-(methylthio)pyrido[2,3-d]pyrimidin-7(8H)-one (17) (0.96 g, 3.19 mmol) in dichloromethane (40 mL) was added 3-chloroperbenzoic acid (77%, 0.68 g, 3.04 mmol) in dichloromethane (10 mL) at 0-5° C. and the mixture was stirred at 0-5° C. for 1 h. The reaction mixture was washed with 10% sodium bicarbonate solution (1×20 mL) and water (1×20 mL). The organic layer was dried over sodium sulfate, filtered and evaporated. The title compound was obtained as a pale yellow solid (0.98 g, 3.10 mmol, 97%). ESMS m/z 316 (M+H)⁺.

### Step 2: Synthesis of 6-bromo-8-ethyl-2-[4-(4-methyl-piperazin-1-yl)-phenylamino]-8H-pyrido[2,3-d]pyrimidin-7-one (19)

6-Bromo-8-ethyl-2-methanesulfinyl-8H-pyrido[2,3-d]pyrimidin-7-one (18) (600 mg, 1.90 mmol) and 4-(4-methylpiperazino)aniline (363 mg, 1.90 mmol) were stirred at 120° C. for 3 h. The reaction mixture was purified by column chromatography using dichloromethane:methanol (100:3→100:5) to give the title compound (340 mg, 0.77 mmol, 40%) as a yellow solid. ESMS m/z 443 (M+H)⁺; ¹H NMR (400 MHz, CDCl₃) δ ppm 8.47 (s, 1H) 7.92 (s, 1H) 7.51 (d, J=8.8 Hz, 2H) 7.24 (br. s., 1H) 6.96 (d, J=8.8 Hz, 2H) 4.48 (q, J=7.0 Hz, 2H) 3.13-3.29 (m, 4H) 2.53-2.64 (m, 4H) 2.36 (s, 3H) 1.35 (t, J=7.0 Hz, 3H).

### Step 3: Synthesis of 3-chloro-4-{8-ethyl-2-[4-(4-methyl-piperazin-1-yl)-phenylamino]-7-oxo-7,8-dihydropyrido[2,3-d]pyrimidin-6-yl}benzoic acid methyl ester (20)

6-Bromo-8-ethyl-2-[4-(4-methyl-piperazin-1-yl)-phenylamino]-8H-pyrido[2,3-d]pyrimidin-7-one (19) (110 mg, 0.25 mmol), 2-chloro-4-(methoxycarbonyl)benzene boronic acid (58 mg, 0.27 mmol), K₃PO₄ (58 mg, 0.27 mmol) and PdCl₂(dppf) (20 mg, 0.02 mmol) were mixed under argon in a degassed mixture of dimethylformamide and water (20:1, 4.5 mL). The resulting suspension was irradiated for 30 min at 140° C. in a microwave reactor. The reaction mixture was evaporated and the residue was purified by column chromatography, eluting with dichloromethane:methanol (95:5). The title compound (78 mg, 0.15 mmol, 60%) was obtained as a yellow solid. ESMS m/z 533 (M+H)⁺; ¹H NMR (400 MHz, CDCl₃) δ ppm 8.55 (s, 1H) 8.15 (d, J=1.5 Hz, 1H) 7.97 (dd, J=7.9, 1.5 Hz, 1H) 7.51-7.64 (m, 3H) 7.48 (d, J=7.8 Hz, 1H) 7.27 (br. s., 1H) 6.97 (d, J=9.0 Hz, 2H) 4.49 (q, J=7.3 Hz, 2H) 3.95 (s, 3H) 3.18-3.35 (m, 4H) 2.67 (br. s., 4H) 2.42 (br. s., 3H) 1.38 (t, J=7.3 Hz, 3H).

### Step 4: Synthesis of 3-chloro-4-{8-ethyl-2-[4-(4-methyl-piperazin-1-yl)-phenylamino]-7-oxo-7,8-dihydro-pyrido[2,3-d]pyrimidin-6-yl}-benzoic acid hydrazide (21)

3-Chloro-4-{8-ethyl-2-[4-(4-methyl-piperazin-1-yl)-phenylamino]-7-oxo-7,8-dihydro-pyrido[2,3-d]pyrimidin-6-yl}benzoic acid methyl ester (20) (77 mg, 0.14 mmol) in the mixture of ethanol (4 mL) and hydrazine hydrate (1 mL) was heated at reflux for 2 h. The reaction mixture was cooled and the yellow precipitate was collected and washed with 2-propanol and diethyl ether to afford the title compound (40 mg, 0.08 mmol, 57%) as a yellow solid. ESMS m/z 533 (M+H)⁺; ¹H NMR (400 MHz, DMSO-d₆) δ ppm 9.94 (br. s., 2H) 8.77 (s, 1H) 7.95 (d, J=1.5 Hz, 1H) 7.87 (s, 1H) 7.83 (dd, J=7.8, 1.5 Hz, 1H) 7.66 (d, J=9.0 Hz, 2H) 7.51 (d, J=7.8 Hz, 1H) 6.94 (d, J=9.0 Hz, 2H) 4.56 (br. s., 2H) 4.36 (q, J=7.0 Hz, 2H) 3.05-3.15 (m, 4H) 2.42-2.48 (m, 4H) 2.22 (s, 3H) 1.28 (t, J=7.0 Hz, 3H).

### Step 5: Synthesis of 6-(2-chloro-4-(1,3,4-oxadiazol-2-yl)phenyl)-8-ethyl-2-(4-(4-methylpiperazin-1-yl)phenylamino)pyrido[2,3-d]pyrimidin-7(8H)-one (22)

3-Chloro-4-{8-ethyl-2-[4-(4-methyl-piperazin-1-yl)-phenylamino]-7-oxo-7,8-dihydro-pyrido[2,3-d]pyrimidin-6-yl}benzoic acid hydrazide (21) (30 mg, 0.06 mmol) was suspended in triethyl orthoformate (5 mL) to which was added trifluoroacetic acid (1 mL). The resulting reaction mixture was heated at 130° C. for 2 h. The volatiles were removed and the residue was taken up in dichloromethane (1×20 mL) and washed with 10% sodium hydroxide solution (2×10 mL). The organic layer was dried over sodium sulfate, filtered and evaporated. The residue was purified by column chromatography using dichloromethane:methanol (95:5). The title compound (18 mg, 0.03 mmol, 50%) was obtained as a yellow solid. ESMS m/z 543 (M+H)⁺; ¹H NMR (400 MHz, CDCl₃) δ ppm 8.57 (s, 1H) 8.50 (s, 1H) 8.21 (d, J=1.5 Hz, 1H) 8.04 (dd, J=8.0, 1.5 Hz, 1H) 7.62 (s, 1H) 7.52-7.60 (m, 3H) 7.29 (br. s., 1H) 6.98 (d, J=9.0 Hz, 2H) 4.50 (q, J=6.8 Hz, 2H) 3.16-3.27 (m, 4H) 2.56-2.65 (m, 4H) 2.37 (s, 3H) 1.39 (d, J=6.8 Hz, 3H).

### Example 4

### Synthesis of 8-(2-ethanesulfonyl-benzyl)-2-[4-(4-methyl-piperazin-1-yl)-phenylamino]-8H-pyrido[2,3-d]pyrimidin-7-one (25)

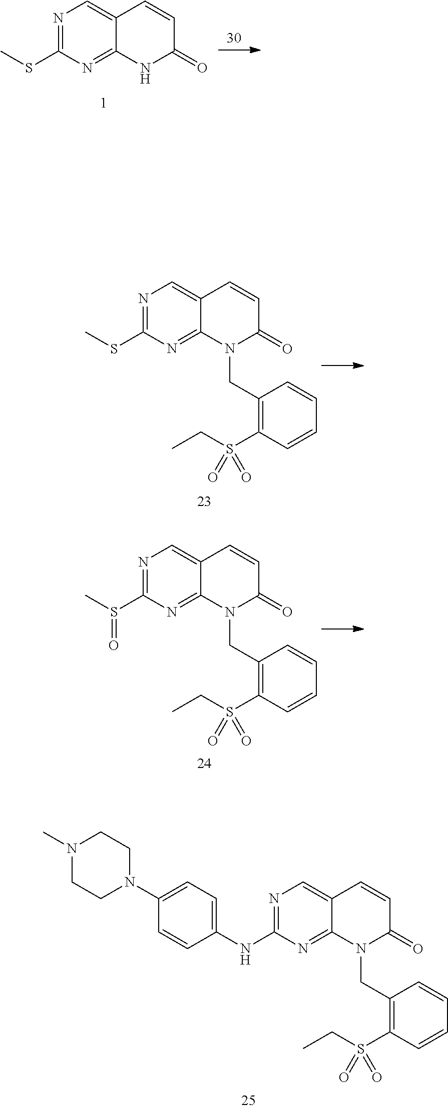 [SMILES: CSc1ncc2ccc(=O)[nH]c2n1 ; CCS(=O)(=O)c1ccccc1Cn1c(=O)ccc2cnc(SC)nc21 ; CCS(=O)(=O)c1ccccc1Cn1c(=O)ccc2cnc(S(C)=O)nc21 ; CCS(=O)(=O)c1ccccc1Cn1c(=O)ccc2cnc(Nc3ccc(N4CCN(C)CC4)cc3)nc21]

### Preparation of Intermediate Compound

### Synthesis of intermediate 1-chloromethyl-2-ethanesulfonyl-benzene (30)

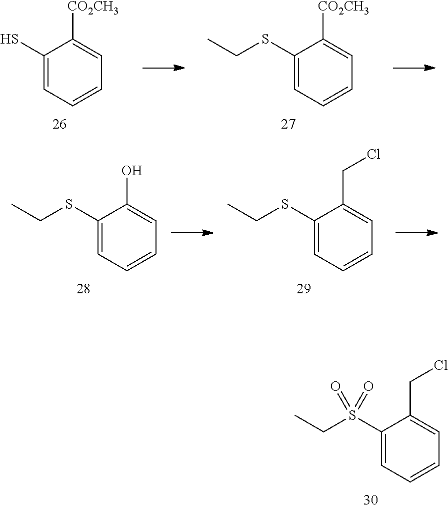

### Step 1: Synthesis of 2-ethylsulfanyl-benzoic acid methyl ester (27)

To a solution of 2-mercaptobenzoic acid methyl ester (26) (1.00 g, 5.95 mmol) in anhydrous dimethylformamide (6 mL) was added K₂CO₃ (1.00 g, 7.24 mmol). The reaction mixture was stirred for 15 min at room temperature, ethyl bromide (662 μl, 8.93 mmol) was added and the reaction was stirred for 1 h. The mixture was poured onto ice (25 g) and extracted with diethyl ether (2×25 mL). The organic layer was dried over sodium sulfate, filtered and evaporated to afford the title compound (1.11 g, 95%) as a yellow oil.

### Step 2: Synthesis of (2-ethylsulfanyl-phenyl)-methanol (28)

To a suspension of lithium aluminum hydride (0.22 g, 5.66 mmol) in anhydrous tetrahydrofuran (22 mL) was added a solution of 2-ethylsulfanyl-benzoic acid methyl ester (27) (1.11 g, 5.65 mmol) in anhydrous tetrahydrofuran (20 mL) at 0° C. and the reaction was stirred for 1 h. Brine (30 mL) was added dropwise to the reaction mixture at 0° C., and the mixture was allowed to warm to room temperature. The two phases were separated and the aqueous layer was extracted with ethyl acetate (2×30 mL). The combined organic layers were dried over sodium sulfate, filtered, and evaporated. The title compound (0.92 g, 5.47 mmol, 97%) was obtained as a yellow oil. ESMS m/z 151 (M+H−H₂O)⁺.

### Step 3: Synthesis of 1-chloromethyl-2-ethylsulfanyl-benzene (29)

(2-Ethylsulfanyl-phenyl)-methanol (28) (0.92 g, 5.47 mmol) was dissolved in anhydrous dichloromethane (15 mL) and thionyl chloride (400 μL, 5.48 mmol) was added. The reaction mixture was heated at reflux for 4 h and evaporated. The title compound (0.99 g, 5.27 mmol, 96%) was obtained as a yellow oil.

### Step 4: Synthesis of 1-chloromethyl-2-ethanesulfonyl-benzene (30)

To a solution of 1-chloromethyl-2-ethylsulfanyl-benzene (29) (0.99 g, 5.27 mmol) in methanol (25 mL) was added a solution of Oxone (3.73 g, 6.07 mmol) in water (27 mL) at room temperature. The reaction mixture was stirred for 18 h. The reaction was evaporated to dryness and the residue taken up in ethyl acetate (30 mL) and water (15 mL). The two phases were separated and the aqueous layer was extracted with ethyl acetate (2×15 mL). The combined organic layers were dried over sodium sulfate, filtered and evaporated to give the title compound (1.09 g, 4.98 mmol, 94%) as a yellow oil. ESMS m/z 236 (M+H+NH₃)⁺; ¹H NMR (400 MHz, CDCl₃) δ ppm 8.04 (d, J=7.8 Hz, 1H) 7.61-7.71 (m, 2H) 7.54 (td, J=7.8, 1.8 Hz, 1H) 5.14 (s, 2H) 3.34 (q, J=7.4 Hz, 2H) 1.30 (t, J=7.4 Hz, 3H).

### Synthesis of 8-(2-ethanesulfonyl-benzyl)-2-[4-(4-methyl-piperazin-1-yl)-phenylamino]-8H-pyrido[2,3-d]pyrimidin-7-one (25)

### Step 1: Synthesis of 8-(2-ethanesulfonyl-benzyl)-2-methylsulfanyl-8H-pyrido[2,3-d]-pyrimidin-7-one (23)

To a suspension of sodium hydride (60% in mineral oil, 204 mg, 5.1 mmol) in anhydrous dimethylformamide (12 mL) was added a solution of 2-methylsulfanyl-8H-pyrido[2,3-d]pyrimidin-7-one (1) (500 mg, 2.59 mmol) in anhydrous dimethylformamide (5 mL) then lithium bromide (590 mg, 6.79 mmol) at 0-5° C. After 30 min, a solution of 1-chloromethyl-2-ethanesulfonyl-benzene (30) (743 mg, 3.39 mmol) in anhydrous dimethylformamide (7 mL) was added slowly and stirring was continued at room temperature for 18 h. The resulting mixture was poured into ice water (150 g) and extracted with ethyl acetate (5×20 mL). The combined organic layers were dried over sodium sulfate, filtered and evaporated. The residue was purified by ISCO using n-hexane:ethyl acetate (1:01:1) as eluent. The title compound (188 mg, 0.50 mmol, 19%) was obtained as a light yellow solid. ESMS m/z 376 (M+H)⁺; ¹H NMR (400 MHz, CDCl₃) δ ppm 8.69 (s, 1H) 8.02-8.08 (m, 1H) 7.72 (d, J=9.5 Hz, 1H) 7.39-7.48 (m, 2H) 6.79-6.88 (m, 1H) 6.69 (d, J=9.5 Hz, 1H) 6.03 (s, 2H) 3.57 (q, J=7.4 Hz, 2H) 2.47 (s, 3H) 1.40 (t, J=7.4 Hz, 3H).

### Step 2: Synthesis of 8-(2-ethanesulfonyl-benzyl)-2-methanesulfinyl-8H-pyrido[2,3-d]-pyrimidin-7-one (24)

To a solution of 8-(2-ethanesulfonyl-benzyl)-2-methylsulfanyl-8H-pyrido[2,3-d]pyrimidin-7-one (23) (183 mg, 0.49 mmol) in dichloromethane (5 mL) was added 3-chloroperbenzoic acid (77%, 101 mg, 0.46 mmol) in dichloromethane (5 mL) at 0-5° C. and the mixture was stirred for 1 h. The reaction mixture was washed with saturated sodium bicarbonate solution (1×10 mL) then with water (1×10 mL). The organic layer was dried over sodium sulfate, filtered and evaporated to yield the title compound as an off-white solid (191 mg, 0.49 mmol, 100%). ESMS m/z 392 (M+H)⁺.

### Step 3: Synthesis of 8-(2-ethanesulfonyl-benzyl)-2-[4-(4-methyl-piperazin-1-yl)-phenylamino]-8H-pyrido[2,3-d]pyrimidin-7-one (25)

8-(2-Ethanesulfonyl-benzyl)-2-methanesulfinyl-8H-pyrido[2,3-d]pyrimidin-7-one (24) (181 mg, 0.46 mmol) and 4-(4-methylpiperazino)aniline (80 mg, 0.90 mmol) were stirred at 120° C. for 4 h. The reaction mixture was purified by column chromatography using dichloromethane:methanol (95:5) as eluent and the solid product was suspended in diisopropyl ether, filtered, and collected to give the title compound (50 mg, 0.10 mmol, 22%) as a light yellow solid. ESMS m/z 519 (M+H)⁺; ¹H NMR (400 MHz, CDCl₃) δ ppm 8.58 (s, 1H) 8.05 (dd, J=7.4, 1.9 Hz, 1H) 7.62 (d, J=9.3 Hz, 1H) 7.39-7.47 (m, 2H) 7.31 (d, J=8.5 Hz, 2H) 7.20 (s, 1H) 6.90 (d, J=8.5 Hz, 1H) 6.82 (d, J=8.5 Hz, 2H) 6.46 (d, J=9.3 Hz, 1H) 5.98 (s, 2H) 3.48 (q, J=7.4 Hz, 2H) 3.11-3.21 (m, 4H) 2.51-2.62 (m, 4H) 2.34 (s, 3H) 1.37 (t, J=7.4 Hz, 3H).

### Example 5

### Synthesis of 8-ethyl-6-(4-methanesulfonyl-phenyl)-2-[4-(4-methyl-piperazin-1-yl)-phenylamino]-8H-pyrido[2,3-d]pyrimidin-7-one (31)

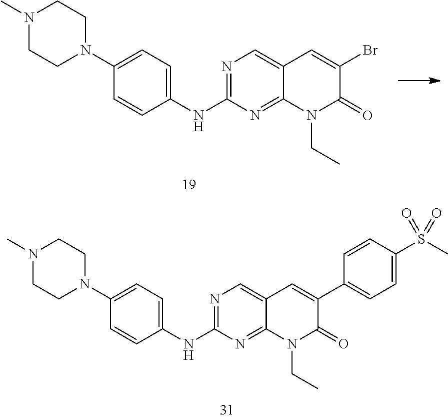

6-Bromo-8-ethyl-2-[4-(4-methyl-piperazin-1-yl)-phenylamino]-8H-pyrido[2,3-d]pyrimidin-7-one (19) (prepared as in Example 3) (120 mg, 0.27 mmol), 4-methylsulfonylphenylboronic acid (60 mg, 0.30 mmol), K₃PO₄ (64 mg, 0.30 mmol) and PdCl₂(dppf) (25 mg, 0.03 mmol) were mixed under argon in a degassed mixture of dimethylformamide and water (20:1, 4 mL). The resulting suspension was irradiated for 30 min at 140° C. in a microwave reactor. After completion, the reaction mixture was evaporated and the residue purified by ISCO eluting with chloroform:methanol (100:0→98:2). The partially purified product (50 mg) was recrystallized from ethyl acetate. The title compound (21 mg, 0.04 mmol, 15%) was obtained as a yellow solid. ESMS m/z 519 (M+H)⁺; ¹H NMR (400 MHz, CDCl₃) δ ppm 8.60 (s, 1H) 7.99 (d, J=8.3 Hz, 2H) 7.90 (d, J=8.3 Hz, 2H) 7.71 (s, 1H) 7.55 (d, J=8.8 Hz, 2H) 7.30 (br. s, 1H) 6.98 (d, J=8.8 Hz, 2H) 4.51 (q, J=7.0 Hz, 2H) 3.18-3.27 (m, 4H) 3.07 (s, 3H) 2.54-2.67 (m, 4H) 2.37 (s, 3H) 1.39 (t, J=7.0 Hz, 3H).

### Example 6

### Synthesis of 8-(2-cyclopropyl-6-fluorobenzyl)-2-[4-(4-methyl-piperazin-1-yl)-phenylamino]-6-prop-1-ynyl-8H-pyrido[2,3-d]pyrimidin-7-one (36)

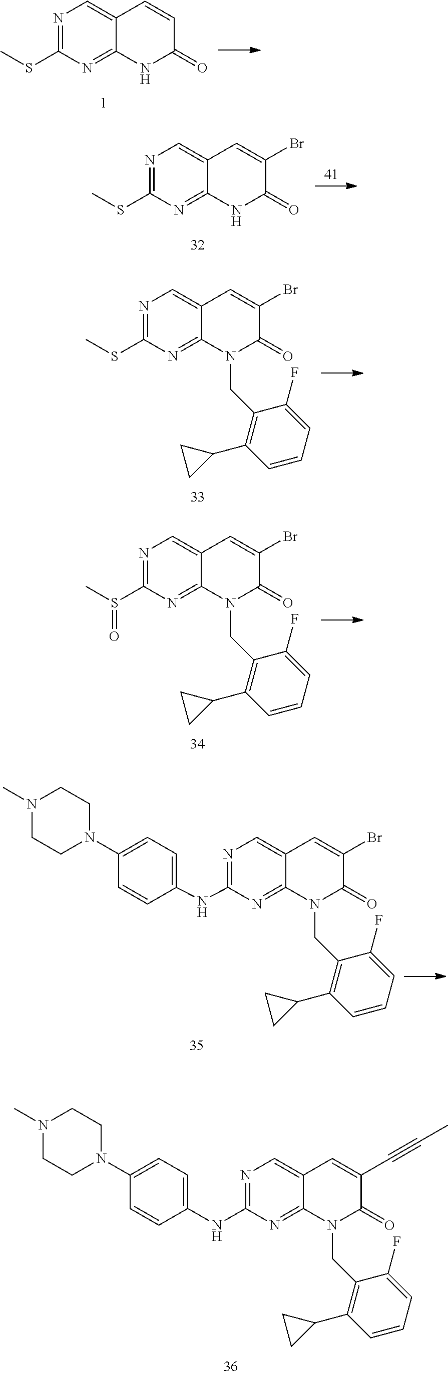 [SMILES: CSc1ncc2ccc(=O)[nH]c2n1 ; CSc1ncc2cc(Br)c(=O)[nH]c2n1 ; CSc1ncc2cc(Br)c(=O)n(Cc3c(F)cccc3C3CC3)c2n1 ; CS(=O)c1ncc2cc(Br)c(=O)n(Cc3c(F)cccc3C3CC3)c2n1 ; CN1CCN(c2ccc(Nc3ncc4cc(Br)c(=O)n(Cc5c(F)cccc5C5CC5)c4n3)cc2)CC1 ; CC#Cc1cc2cnc(Nc3ccc(N4CCN(C)CC4)cc3)nc2n(Cc2c(F)cccc2C2CC2)c1=O]

### Preparation of Intermediate Compound

### Synthesis of intermediate 2-chloromethyl-1-cyclopropyl-3-fluorobenzene (41)

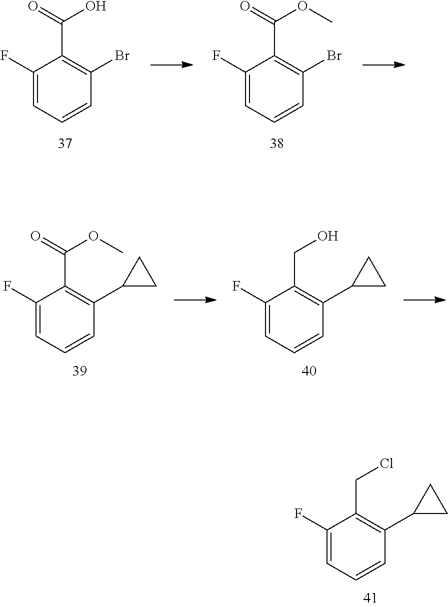

### Step 1: Synthesis of methyl 2-bromo-6-fluorobenzoate (38)

2-Bromo-6-fluorobenzoic acid (37) (12.50 g, 57 mmol) was dissolved in a mixture of methanol (60 mL) and conc. sulfuric acid (65 mL). The solution was heated to 80° C. and stirred for 12 h. The reaction mixture was cooled and 20% sodium carbonate solution (500 mL) was added slowly to reach pH=8. The mixture was extracted with dichloromethane (3×180 mL), and the combined organic layers were dried over magnesium sulfate and evaporated. The title compound (11.29 g, 48.5 mmol, 85%) was obtained as a brown oil.

### Step 2: Synthesis of methyl 2-cyclopropyl-6-fluorobenzoate (39)

Methyl 2-bromo-6-fluorobenzoate (38) (11.29 g, 48.5 mmol), cyclopropylboronic acid (6.24 g, 72.7 mmol), K₃PO₄ (30.85 g, 145.4 mmol) and Pd(PPh₃)₄ (2.80 g, 2.4 mmol) were mixed under argon in a degassed mixture of toluene and water (20:1, 160 mL). The resulting brown suspension was heated at reflux for 2.5 h, cooled, filtered through Celite, and evaporated. The residue was partitioned between ethyl acetate (150 mL) and brine (150 mL). The layers were separated and organic layer was dried over magnesium sulfate and evaporated. The oily residue was suspended in hexane and the solid was removed by filtration. Evaporation of the filtrate afforded the title compound (9.30 g, 47.9 mmol, 99%) as a light brown oil.

### Step 3: Synthesis of (2-cyclopropyl-6-fluorophenyl)-methanol (40)

To a solution of methyl 2-cyclopropyl-6-fluorobenzoate (39) (4.54 g, 23.4 mmol) in anhydrous tetrahydrofuran (30 mL) at room temperature was added lithium aluminium hydride (1.42 g, 37.4 mmol) in small portions and the mixture was stirred overnight. The reaction mixture was cooled to 0° C. and water (3 mL) was added dropwise, the mixture was warmed to room temperature and an additional portion of water (30 mL) was added. The solids were removed by filtration, and the aqueous filtrate was extracted with ethyl acetate (2×30 mL). The combined organic layers were dried over magnesium sulfate and evaporated. Purification by column chromatography over silica using n-hexane:ethyl acetate (8:1) as eluent afforded the title compound (3.28 g, 19.8 mmol, 84%) as a pale yellow oil.

### Step 4: Synthesis of 2-chloromethyl-1-cyclopropyl-3-fluorobenzene (41)

(2-Cyclopropyl-6-fluorophenyl)-methanol (40) (5.56 g, 34 mmol) was dissolved in dichloromethane (100 mL) and thionyl chloride (2.49 mL, 34 mmol) was added. The mixture was refluxed for 4 h and evaporated. The crude product was purified by column chromatography over silica, eluting with n-hexane:ethyl acetate (95:5). The title compound (3.41 g, 18.4 mmol, 54%) was isolated as a pale yellow oil.

### Synthesis of 8-(2-cyclopropyl-6-fluorobenzyl)-2-[4-(4-methyl-piperazin-1-yl)-phenylamino]-6-prop-1-ynyl-8H-pyrido[2,3-d]pyrimidin-7-one (36)

### Step 1: Synthesis of 6-bromo-2-(methylthio)pyrido[2,3-d]pyrimidin-7(8H)-one (32)

To a solution of 2-methylsulfanyl-8H-pyrido[2,3-d]pyrimidin-7-one (1) (1.00 g, 5.2 mmol) in anhydrous dimethylformamide (25 mL) was added N-bromosuccinimide (0.99 g, 5.6 mmol) portionwise at room temperature, and the reaction mixture was stirred for 18 h. The mixture was concentrated, and the solid was triturated with hot water (1×20 mL), filtered, and washed with isopropanol to give title compound as a pale yellow solid (0.68 g, 2.5 mmol, 48%). ESMS m/z 272 (M+H)⁺; ¹H NMR (400 MHz, DMSO-d₆) δ ppm 12.88 (br. s, 1H), 8.84 (s, 1H), 8.47 (s, 1H), 2.57 (s, 3H).

### Step 2: Synthesis of 6-bromo-8-(2-cyclopropyl-6-fluoro-benzyl)-2-methylsulfanyl-8H-pyrido[2,3-d]pyrimidin-7-one (33)

To a solution of 6-bromo-2-(methylthio)pyrido[2,3-d]pyrimidin-7(8H)-one (32) (7, 2.12 g, 7.8 mmol) in anhydrous dimethylformamide (40 mL) sodium hydride (60% on mineral oil, 467 mg, 11.7 mmol) was added and the reaction was stirred at room temperature for 30 min. A solution of 2-chloromethyl-1-cyclopropyl-3-fluorobenzene (41) (1.58 g, 8.6 mmol) in anhydrous dimethylformamide (10 mL) was added slowly and the reaction was stirred for 18 h. The mixture was poured onto ice water (250 g) and extracted with dichloromethane (3×80 mL). The combined organic layers were dried over magnesium sulfate, filtered and evaporated. The residue was triturated with n-hexane, the precipitated product was collected, washed with methanol, then diethyl ether. The crude product was recrystallized from ethyl acetate to give the title compound (1.31 g, 3.1 mmol, 54%) as a pale yellow solid. ESMS m/z 420 (M+H)⁺.

### Step 3: Synthesis of 6-bromo-8-(2-cyclopropyl-6-fluorobenzyl)-2-methanesulfinyl-8H-pyrido[2,3-d]pyrimidin-7-one (34)

To a solution of 6-bromo-8-(2-cyclopropyl-6-fluorobenzyl)-2-methylsulfanyl-8H-pyrido[2,3-d]pyrimidin-7-one (33) (500 mg, 1.2 mmol) in dichloromethane (20 mL) was added 3-chloroperbenzoic acid (77%, 253 mg, 1.1 mmol) in dichloromethane (15 mL) at 0-5° C. and the mixture was stirred at 0-5° C. for 1 h. The reaction mixture was washed with saturated sodium bicarbonate solution (2×30 mL) then with brine (30 mL). The organic layer was dried over sodium sulfate, filtered and evaporated. The crude product was purified by column chromatography eluting with chloroform to yield the title compound as a white solid (385 mg, 0.9 mmol, 74%). ESMS m/z 436 (M+H)⁺.

### Step 4: Synthesis of 6-bromo-8-(2-cyclopropyl-6-fluorobenzyl)-2-[4-(4-methyl-piperazin-1-yl)-phenylamino]-8H-pyrido[2,3-d]pyrimidin-7-one (35)

6-Bromo-8-(2-cyclopropyl-6-fluorobenzyl)-2-methanesulfinyl-8H-pyrido[2,3-d]pyrimidin-7-one (34) (385 mg, 0.9 mmol) and 4-(4-methylpiperazino)aniline (169 mg, 0.9 mmol) were stirred at 140° C. for 6 h. The reaction mixture was purified by column chromatography using dichloromethane:methanol (100:1→100:3→100:5) to yield the title compound as a yellow solid (272 mg, 0.48 mmol, 55%). ESMS m/z 563 (M+H)

### Step 5: Synthesis of 8-(2-cyclopropyl-6-fluorobenzyl)-2-[4-(4-methyl-piperazin-1-yl)-phenylamino]-6-prop-1-ynyl-8H-pyrido[2,3-d]pyrimidin-7-one (36)

To a solution of 6-bromo-8-(2-cyclopropyl-6-fluorobenzyl)-2-[4-(4-methyl-piperazin-1-yl)-phenylamino]-8H-pyrido[2,3-d]pyrimidin-7-one (35) (130 mg, 0.23 mmol) in degassed dioxane (5 mL), tributyl(1-propynyl)-tin (105 μl, 0.34 mmol) and Pd(PPh₃)₄ (27 mg, 0.02 mmol) was added. The resulting mixture was irradiated at 100° C. for 90 min and evaporated. The solid residue was dissolved in dichloromethane (15 mL) and was extracted with saturated sodium bicarbonate (2×15 mL). The combined aqueous layers were back-extracted with dichloromethane (15 mL). The combined organic layers were dried over sodium sulfate and evaporated. The residue was dissolved in dichloromethane and filtered through a silica pad which was washed with dichloromethane:methanol (9:1). The crude product was purified by preparative HPLC to give the title compound (25 mg, 0.05 mmol, 22%) as a yellow solid. ESMS m/z 522 (M+H)⁺; ¹H NMR (400 MHz, CDCl₃) δ ppm 8.48 (s, 1H) 7.67 (s, 1H) 7.37 (d, J=9.0 Hz, 2H) 7.15 (br. s, 1H) 7.07-7.14 (m, 1H) 6.99 (s, 1H) 6.83-6.91 (m, 3H) 6.75-6.82 (m, 1H) 5.81 (s, 2H) 3.26 (br. s, 4H) 2.73 (br. s, 4H) 2.47 (br. s, 3H) 2.19-2.27 (m, 1H) 2.09 (s, 3H) 0.81-0.87 (m, 2H) 0.62-0.69 (m, 2H).

### Biological Examples

### Example 7

### In vitro PAK Inhibition Assay

Assay Conditions

Compounds are screened in 1% DMSO (final) in the well. For 10 point titrations, 3-fold serial dilutions are conducted. All Peptide/Kinase Mixtures are diluted to a 2× working concentration in the appropriate Kinase Buffer

Kinase Specific Assay Conditions

PAK1

The 2×PAK1/Ser/Thr 19 mixture is prepared in 50 mM HEPES pH 7.5, 0.01% BRIJ-35, 10 mM MgCl₂, 1 mM EGTA. The final 10 μL Kinase Reaction consists of 2.71-30.8 ng PAK1 and 2 μM Ser/Thr 19 in 50 mM HEPES pH 7.5, 0.01% BRIJ-35, 10 mM MgCl₂, 1 mM EGTA. After the 1 hour Kinase Reaction incubation, 5 μL of a 1:128 dilution of Development Reagent A is added.

PAK2 (PAK65)

The 2×PAK2 (PAK65)/Ser/Thr 20 mixture is prepared in 50 mM HEPES pH 7.5, 0.01% BRIJ-35, 10 mM MgCl₂, 1 mM EGTA. The final 10 μL Kinase Reaction consists of 0.29-6 ng PAK2 (PAK65) and 2 μM Ser/Thr 20 in 50 mM HEPES pH 7.5, 0.01% BRIJ-35, 10 mM MgCl₂, 1 mM EGTA. After the 1 hour Kinase Reaction incubation, 5 μL of a 1:256 dilution of Development Reagent A is added.

PAK3

The 2×PAK3/Ser/Thr 20 mixture is prepared in 50 mM HEPES pH 7.5, 0.01% BRIJ-35, 10 mM MgCl₂, 1 mM EGTA. The final 10 μL Kinase Reaction consists of 2.25-22 ng PAK3 and 2 μM Ser/Thr 20 in 50 mM HEPES pH 7.5, 0.01% BRIJ-35, 10 mM MgCl₂, 1 mM EGTA. After the 1 hour Kinase Reaction incubation, 5 μL of a 1:256 dilution of Development Reagent A is added.

PAK-4

The 2X PAK-4/Ser/Thr 20 mixture is prepared in 50 mM HEPES pH 7.5, 0.01% BRIJ-35, 10 mM MgCl₂, 1 mM EGTA. The final 10 μL Kinase Reaction consists of 0.1-0.75 ng PAK-4 and 2 μM Ser/Thr 20 in 50 mM HEPES pH 7.5, 0.01% BRIJ-35, 10 mM MgCl₂, 1 mM EGTA. After the 1 hour Kinase Reaction incubation, 5 μL of a 1:256 dilution of Development Reagent A is added.

### Assay Controls

The following controls are made for each individual kinase and are located on the same plate as the kinase:

0% Phosphorylation Control (100% Inhibition Control)

The maximum Emission Ratio is established by the 0% Phosphorylation Control (100% Inhibition Control), which contains no ATP and therefore exhibits no kinase activity. This control yields 100% cleaved peptide in the Development Reaction.

100% Phosphorylation Control

The 100% Phosphorylation Control, which consists of a synthetically phosphorylated peptide of the same sequence as the peptide substrate, is designed to allow for the calculation of percent phosphorylation.

This control yields a very low percentage of cleaved peptide in the Development Reaction.

The 0% Phosphorylation and 100% Phosphorylation Controls allow one to calculate the percent Phosphorylation achieved in a specific reaction well. Control wells do not include any kinase inhibitors.

0% Inhibition Control

The minimum Emission Ratio in a screen is established by the 0% Inhibition Control, which contains active kinase. This control is designed to produce a 10-50%*phosphorylated peptide in the Kinase Reaction.

Known Inhibitor

A known inhibitor control standard curve, 10 point titration, is run for each individual kinase on the same plate as the kinase to ensure the kinase is inhibited within an expected IC50 range previously determined.

The following controls are prepared for each concentration of Test Compound assayed:

Development Reaction Interference

The Development Reaction Interference is established by comparing the Test Compound Control wells that do not contain ATP versus the 0% Phosphorylation Control (which does not contain the Test Compound). The expected value for a non-interfering compound should be 100%. Any value outside of 90% to 110% is flagged.

Test Compound Fluorescence Interference

The Test Compound Fluorescence Interference is determined by comparing the Test Compound Control wells that do not contain the Kinase/Peptide Mixture (zero peptide control) versus the 0% Inhibition Control. The expected value for a non-fluorescence compound should be 0%. Any value >20% is flagged.

Assay Protocol

Bar-coded Corning, low volume NBS, black 384-well plate (Corning Cat. #3676)
1. Add the following solutions to a well in a 384-well plate: 2.5 μL of 4× Test Compound OR (100 nL 100× Test Compound plus 2.4 μL kinase buffer)
   5 μL of 2× Peptide/Kinase (PAK) Mixture
   2.5 μL of 4×ATP Solution
2. Shake the plate for 30-seconds
3. Incubate the PAK Kinase Reaction at room temperature for 60-minutes
4. Add 5 μL of Development Reagent Solution to each well
5. Shake the plate for 30-seconds
6. Incubate the Development Reaction for 60-minutes
7. Determine the fluorescence using a fluorescence plate reader
8. Analyze the fluorescence data

Data Analysis

The following equations are used for each set of data points:

| | Equation |
|---|---|
| Correction for Background Fluorescence | FI [symbol] − FI [symbol] |
| Emission Ratio (using values corrected for background fluorescence) | $\frac{Coumarin Emission ( 445 nm )}{Fluorescein Emission ( 520 nm )}$ |
| % Phosphorylation (% Phos) | $\{ 1 - \frac{( Emission Ratio \times \text{?} ) - \text{?}}{( \text{?} - \text{?} ) + [ Emission Ratio \times ( \text{?} - \text{?} ) ]} \} \text{?} 100$ $\text{?} \text{indicates text missing or illegible when filed}$ |
| % Inhibition | $\{ 1 - \frac{\% \text{?}}{\% \text{?}} \} \text{?} 100$ $\text{?} \text{indicates text missing or illegible when filed}$ |
| Z [symbol] (using Emission Ratio values) | $1 - \frac{3 \text{?}}{\text{?} - \text{?}}$ $\text{?} \text{indicates text missing or illegible when filed}$ |
| Difference Between Data Points | \|% Inhibition [symbol] − % Inhibition [symbol] \| |
| (single point only) | |
| Development Reaction Interference (DRI) (no ATP control) | $\frac{Emission \text{?}}{Emission \text{?}}$ $\text{?} \text{indicates text missing or illegible when filed}$ |
| Test Compound Fluorescence Interference (TCFI) (check both Coumarin and Fluorescein emissions) | $\frac{\text{?}}{\text{?}}$ $\text{?} \text{indicates text missing or illegible when filed}$ |

FI = Fluorescence Intensity
C100% = Average Coumarin emission signal of the 100% Phos. Control
C0% = Average Coumarin emission signal of the 0% Phos. Control
F100% = Average Fluorescein emission signal of the 100% Phos. Control
F0% = Average Fluorescein emission signal of the 0% Phos. Control
DRI = Development Reaction Interference
TCFI = Test Compound Fluorescence Interference [symbol]
indicates data missing or illegible when filed

Graphing Software

SelectScreen® Kinase Profiling Service uses XLfit from IDBS. The dose response curve is curve fit to model number 205 (sigmoidal dose-response model). If the bottom of the curve does not fit between −20% & 20% inhibition, it is set to 0% inhibition. If the top of the curve does not fit between 70% and 130% inhibition, it is set to 100% inhibition.

Table of Kinase ATP Km Bins and Inhibitor Validation

The table below provides specifications and data around each kinase. The representative 1050 value with a known inhibitor for each kinase was determined at the ATP bin nearest to the ATP Km app.

| | Z′-LYTE | ATP Km app | ATP Bin | | IC50 |
|---|---|---|---|---|---|
| Kinase | Substrate | (μM) | (μM) | Inhibitor | (nM) |
| PAK1 | Ser/Thr 19 | 48.5 | 50 | Staurosporine | 3.00 |
| PAK2 | Ser/Thr 20 | 89 | 75 | Staurosporine | 30.0 |
| (PAK65) | | | | | |
| PAK3 | Ser/Thr 20 | 101 | 100 | Staurosporine | 15.3 |
| PAK4 | Ser/Thr 20 | 3 | 5 | Staurosporine | 9.71 |

TABLE
PAK Inhibition IC50

| | PAK1 | PAK2 | PAK3 | PAK4 |
|---|---|---|---|---|
| Compound | IC50 (nM) | IC50 (nM) | IC50 (nM) | IC50 (nM) |
| 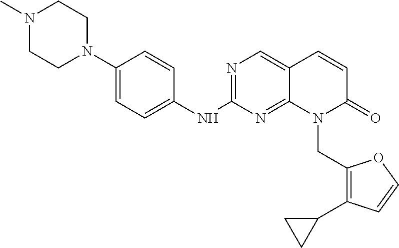 [SMILES: CN1CCN(c2ccc(Nc3ncc4ccc(=O)n(Cc5occc5C5CC5)c4n3)cc2)CC1] | C | C | C | C |
| 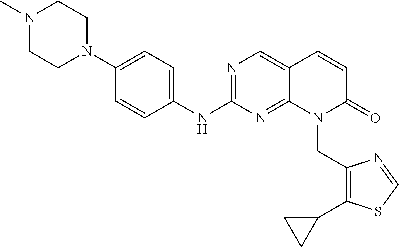 [SMILES: CN1CCN(c2ccc(Nc3ncc4ccc(=O)n(Cc5ncsc5C5CC5)c4n3)cc2)CC1] | B | C | C | C |
| 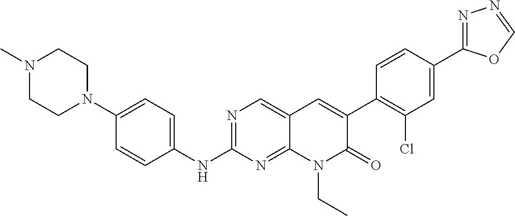 [SMILES: CCn1c(=O)c(-c2ccc(-c3nnco3)cc2Cl)cc2cnc(Nc3ccc(N4CCN(C)CC4)cc3)nc21] | A | A | A | C |
| 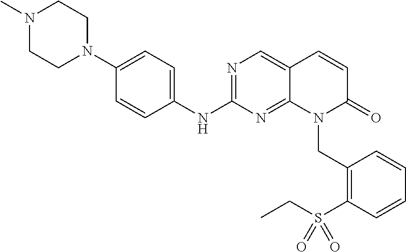 [SMILES: CCS(=O)(=O)c1ccccc1Cn1c(=O)ccc2cnc(Nc3ccc(N4CCN(C)CC4)cc3)nc21] | B | B | C | C |
| 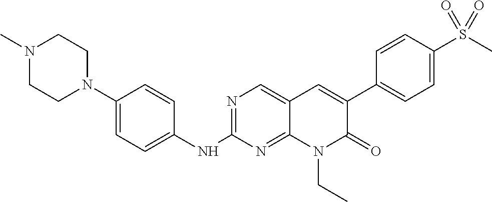 | D | D | D | D |
| 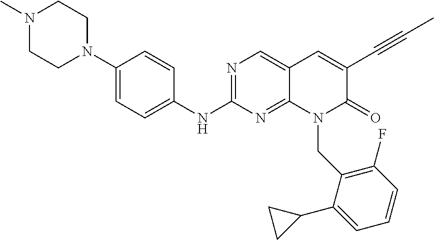 [SMILES: CC#Cc1cc2cnc(Nc3ccc(N4CCN(C)CC4)cc3)nc2n(Cc2c(F)cccc2C2CC2)c1=O] | B | B | B | C |
| 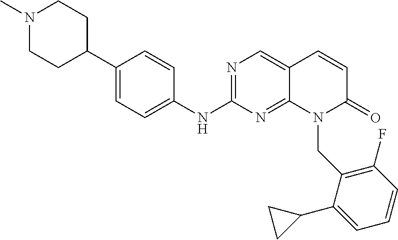 [SMILES: CN1CCC(c2ccc(Nc3ncc4ccc(=O)n(Cc5c(F)cccc5C5CC5)c4n3)cc2)CC1] | A | B | B | B |
| 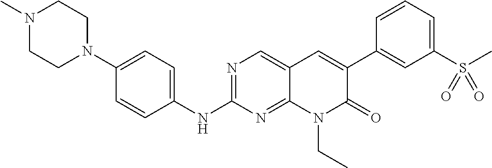 | B | C | B | D |
| 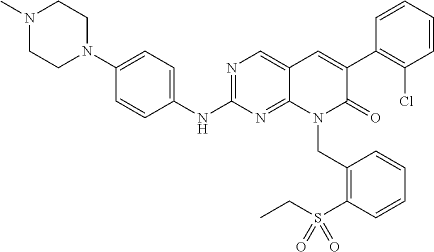 [SMILES: CCS(=O)(=O)c1ccccc1Cn1c(=O)c(-c2ccccc2Cl)cc2cnc(Nc3ccc(N4CCN(C)CC4)cc3)nc21] | A | A | A | C |
| 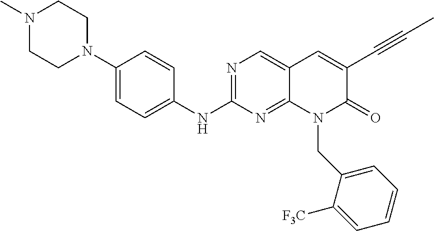 [SMILES: CC#Cc1cc2cnc(Nc3ccc(N4CCN(C)CC4)cc3)nc2n(Cc2ccccc2C(F)(F)F)c1=O] | B | B | C | C |
| 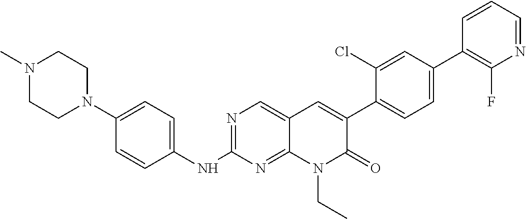 [SMILES: CCn1c(=O)c(-c2ccc(-c3cccnc3F)cc2Cl)cc2cnc(Nc3ccc(N4CCN(C)CC4)cc3)nc21] | A | A | A | B |
| 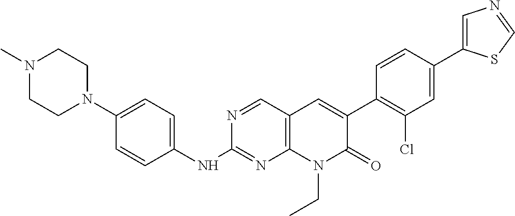 [SMILES: CCn1c(=O)c(-c2ccc(-c3cncs3)cc2Cl)cc2cnc(Nc3ccc(N4CCN(C)CC4)cc3)nc21] | A | A | A | D |
| 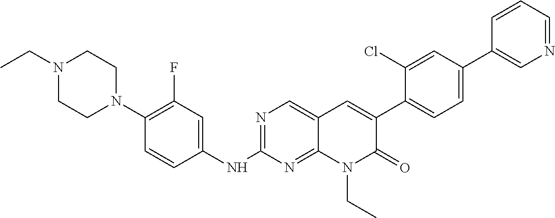 [SMILES: CCN1CCN(c2ccc(Nc3ncc4cc(-c5ccc(-c6cccnc6)cc5Cl)c(=O)n(CC)c4n3)cc2F)CC1] | A | A | A | D |
| 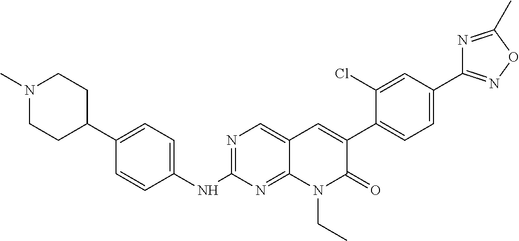 [SMILES: CCn1c(=O)c(-c2ccc(-c3noc(C)n3)cc2Cl)cc2cnc(Nc3ccc(C4CCN(C)CC4)cc3)nc21] | A | A | A | C |
| 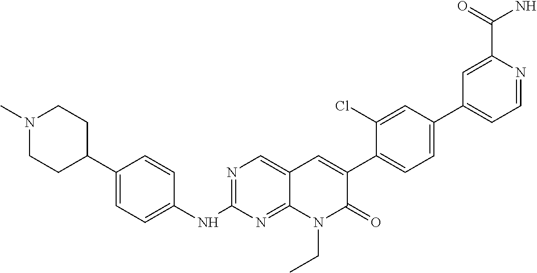 [SMILES: CCn1c(=O)c(-c2ccc(-c3ccnc(C(N)=O)c3)cc2Cl)cc2cnc(Nc3ccc(C4CCN(C)CC4)cc3)nc21] | B | B | B | D |
| 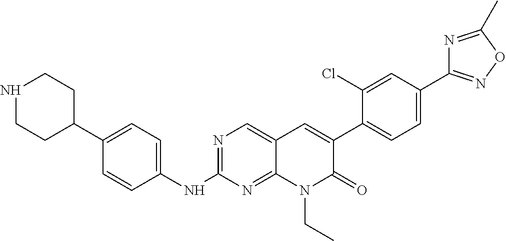 [SMILES: CCn1c(=O)c(-c2ccc(-c3noc(C)n3)cc2Cl)cc2cnc(Nc3ccc(C4CCNCC4)cc3)nc21] | A | A | B | C |
| 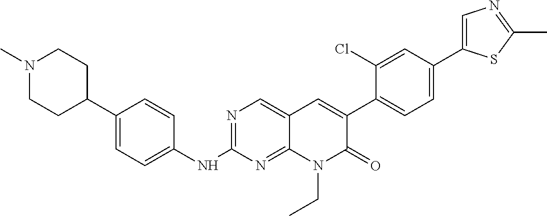 | A | A | A | C |
| 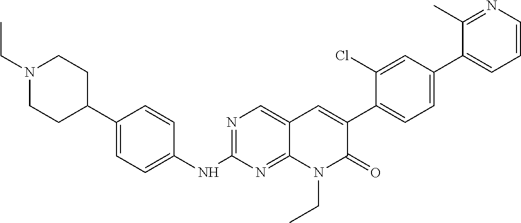 [SMILES: CCN1CCC(c2ccc(Nc3ncc4cc(-c5ccc(-c6cccnc6C)cc5Cl)c(=O)n(CC)c4n3)cc2)CC1] | A | A | A | D |
| 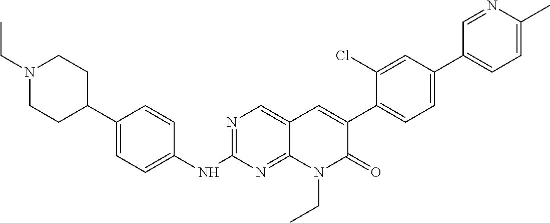 [SMILES: CCN1CCC(c2ccc(Nc3ncc4cc(-c5ccc(-c6ccc(C)nc6)cc5Cl)c(=O)n(CC)c4n3)cc2)CC1] | A | A | A | C |
| 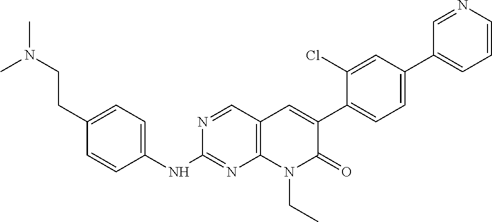 [SMILES: CCn1c(=O)c(-c2ccc(-c3cccnc3)cc2Cl)cc2cnc(Nc3ccc(CCN(C)C)cc3)nc21] | A | A | A | C |
| 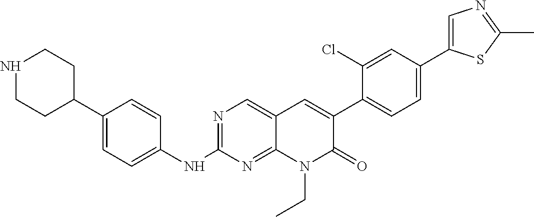 [SMILES: CCn1c(=O)c(-c2ccc(-c3cnc(C)s3)cc2Cl)cc2cnc(Nc3ccc(C4CCNCC4)cc3)nc21] | A | A | A | C |
| 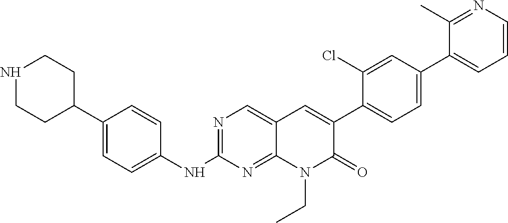 [SMILES: CCn1c(=O)c(-c2ccc(-c3cccnc3C)cc2Cl)cc2cnc(Nc3ccc(C4CCNCC4)cc3)nc21] | A | A | A | D |
| 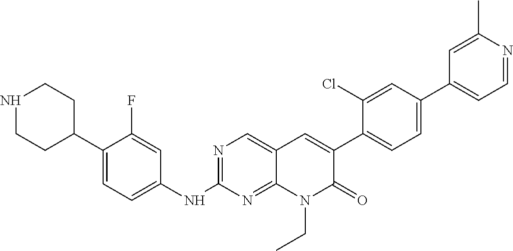 [SMILES: CCn1c(=O)c(-c2ccc(-c3ccnc(C)c3)cc2Cl)cc2cnc(Nc3ccc(C4CCNCC4)c(F)c3)nc21] | A | A | B | C |
| 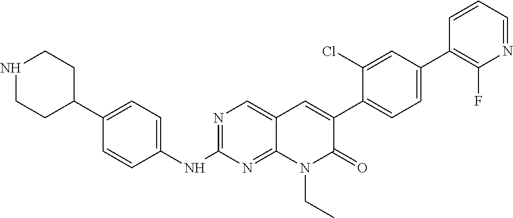 [SMILES: CCn1c(=O)c(-c2ccc(-c3cccnc3F)cc2Cl)cc2cnc(Nc3ccc(C4CCNCC4)cc3)nc21] | A | A | A | B |
| 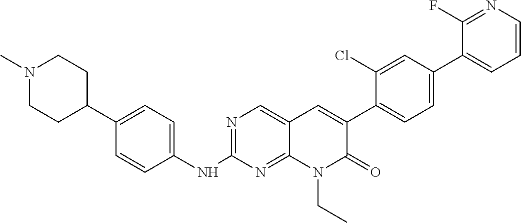 [SMILES: CCn1c(=O)c(-c2ccc(-c3cccnc3F)cc2Cl)cc2cnc(Nc3ccc(C4CCN(C)CC4)cc3)nc21] | A | A | A | B |
| 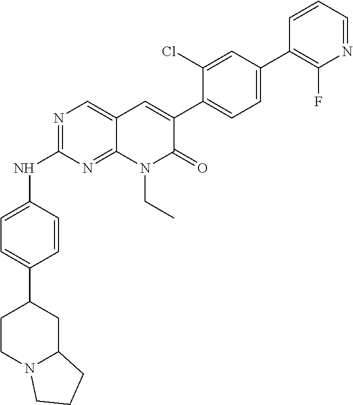 [SMILES: CCn1c(=O)c(-c2ccc(-c3cccnc3F)cc2Cl)cc2cnc(Nc3ccc(C4CCN5CCCC5C4)cc3)nc21] | A | A | A | C |
| 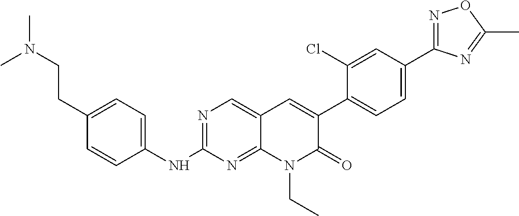 | A | A | A | C |
| 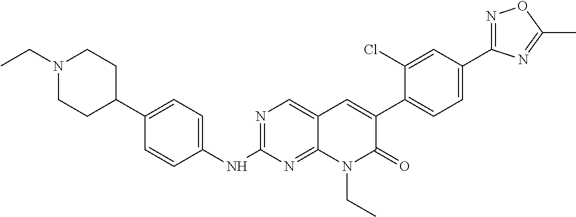 [SMILES: CCN1CCC(c2ccc(Nc3ncc4cc(-c5ccc(-c6noc(C)n6)cc5Cl)c(=O)n(CC)c4n3)cc2)CC1] | A | A | A | C |
| 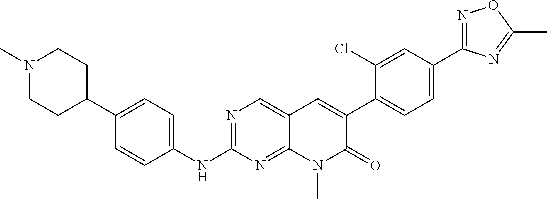 | A | A | A | D |
| 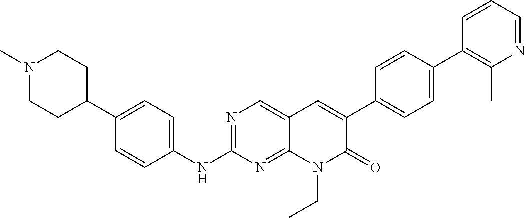 [SMILES: CCn1c(=O)c(-c2ccc(-c3cccnc3C)cc2)cc2cnc(Nc3ccc(C4CCN(C)CC4)cc3)nc21] | C | C | C | D |
| 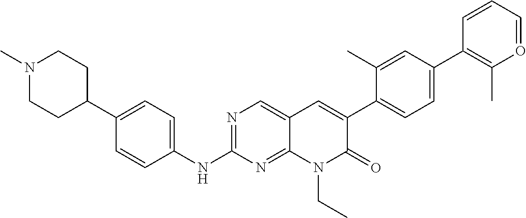 | A | B | B | D |
| 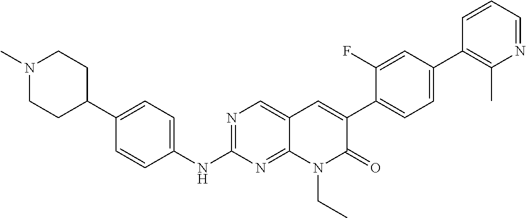 [SMILES: CCn1c(=O)c(-c2ccc(-c3cccnc3C)cc2F)cc2cnc(Nc3ccc(C4CCN(C)CC4)cc3)nc21] | B | B | B | D |
| 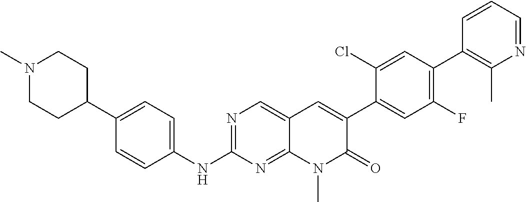 [SMILES: Cc1ncccc1-c1cc(Cl)c(-c2cc3cnc(Nc4ccc(C5CCN(C)CC5)cc4)nc3n(C)c2=O)cc1F] | B | B | C | D |
| 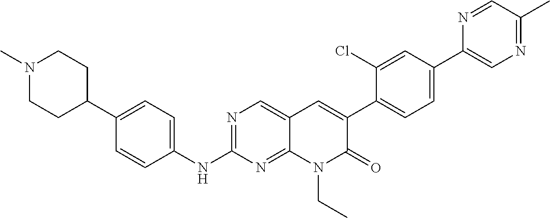 | A | A | A | B |
| 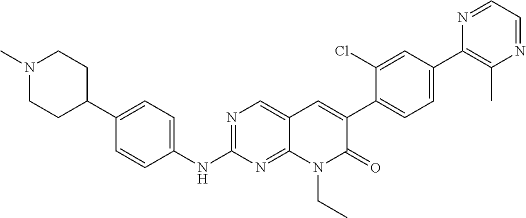 [SMILES: CCn1c(=O)c(-c2ccc(-c3nccnc3C)cc2Cl)cc2cnc(Nc3ccc(C4CCN(C)CC4)cc3)nc21] | A | A | A | C |
| 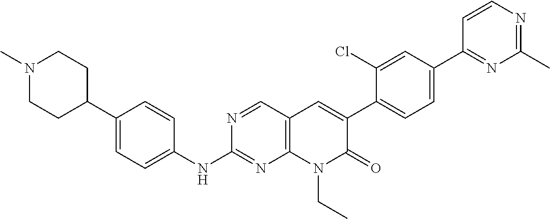 | A | A | A | C |
| 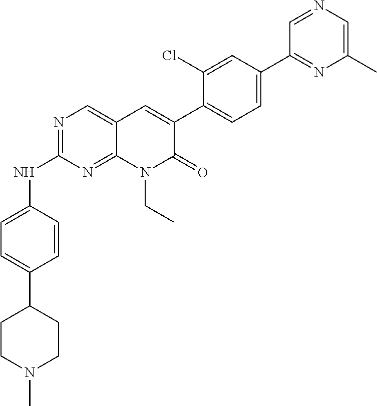 [SMILES: CCn1c(=O)c(-c2ccc(-c3cncc(C)n3)cc2Cl)cc2cnc(Nc3ccc(C4CCN(C)CC4)cc3)nc21] | A | A | A | C |
| 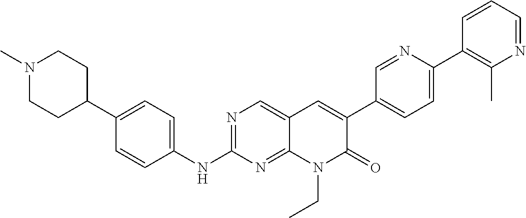 [SMILES: CCn1c(=O)c(-c2ccc(-c3cccnc3C)nc2)cc2cnc(Nc3ccc(C4CCN(C)CC4)cc3)nc21] | D | D | D | D |
| 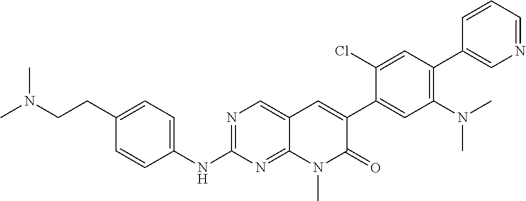 [SMILES: CN(C)CCc1ccc(Nc2ncc3cc(-c4cc(N(C)C)c(-c5cccnc5)cc4Cl)c(=O)n(C)c3n2)cc1] | A | B | A | D |
| 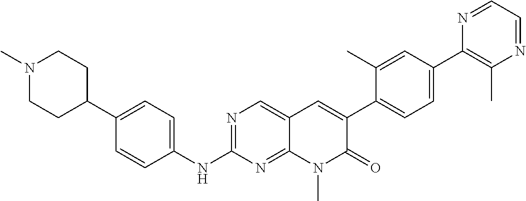 [SMILES: Cc1cc(-c2nccnc2C)ccc1-c1cc2cnc(Nc3ccc(C4CCN(C)CC4)cc3)nc2n(C)c1=O] | A | A | B | D |

A, IC50 < 50 nM;
B, 50 nM ≦ IC50 ≦ 500 nM;
C, 0.5 μM < IC50 < 5 μM;
D, IC50 ≧ 5 μM

### Example 8

### In Vitro p-PAK1(S144) Cellular Assay

Compounds for treatment of cancer are subject to in vitro cellular HTRF assay for p-PAK1(S144). The results of two compounds, as a non-limiting example, are listed in the table below. The cell line is RT4-D6P2T. The HTRF assay kits are obtained from Cisbio Bioassays, 135 South Road, Bedford, Mass. 01730, USA.

| | pPAK1(S144) |
|---|---|
| Compound | IC50 (nM) |
| 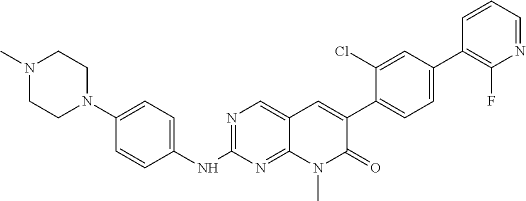 [SMILES: CN1CCN(c2ccc(Nc3ncc4cc(-c5ccc(-c6cccnc6F)cc5Cl)c(=O)n(C)c4n3)cc2)CC1] | B |
| 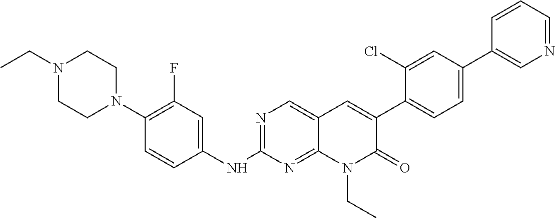 [SMILES: CCN1CCN(c2ccc(Nc3ncc4cc(-c5ccc(-c6cccnc6)cc5Cl)c(=O)n(CC)c4n3)cc2F)CC1] | B |

A, IC50 < 50 nM;
B, 50 nM ≦ IC50 ≦ 500 nM;
C, 0.5 μM < IC50 < 5 μM;
D, IC50 ≧ 5 μM

### Example 9

### Growth Inhibition of a Compound of Formula I-VI in Various Cancer Cell Lines

Methodology: 60 cell lines (CCRF-CEM, HL-60(TB), K-562, MOLT-4, RPMI-8226, SR, A549, EKVX, HOP-62, HOP-92, NCI-H226, NCI-H23, NCI-H322M, NCI-H460, NCI-H522, COLO 205, HCC-2998, HCT-116, HCT-15, HT29, KM12, SW-620, SF-268, SF-295, SF-539, SNB-19, SNB-75, U251, LOX IMV1, MALME-3M, M14, SK-MEL-2, SK− MEL-28, SK-MEL-5, UACC-257, UACC-62, IGR-OV 1, OVCAR-3, OVCAR-4, OVCAR-5, OVCAR-8, SK-OV-3, 786-0, A498, ACHN, CAM-1, RXF 393, SN12C, TK-10, UO-31, PC-3, DU-145, MCF7, NCI/ADR-RES, MDA-MB-231, HS 578T, MDA-MB-435, MDA-MB-468, BT-549, and T-47D) are grown in RPMI-1640 medium with 10% FBS. Stock solutions of a test compound are prepared in DMSO. Concentrations of from about 0.001 μM to about 20 μM of each compound in RPM-1640 media are prepared. The test compound is added to wells containing 50 μL of cells and medium. A CellTiter-Glo (CTG) assay is carried out on the 0 hr plate to obtain a 0 hr count. Cells are exposed to the test compound for 72 hours. Following the exposure period, the plates are assayed using CTG. Luminescence is recorded on Synergy. A test compound described herein exhibited a GLo in various cell lines of less than about 1 μM.

### Example 10

### Clinical Trial of PAK Inhibitor in Children, Adolescents, and Young Adults With Neurofibromatosis Type 1 and Progressive Plexiform Neurofibromas

Purpose

Background:

Neurofibromatosis Type 1 (NF1) is an autosomal dominant, progressive genetic disorder characterized by diverse clinical manifestations. Patients with NF1 have an increased risk of developing tumors of the central and peripheral nervous system including plexiform neurofibromas, which are benign nerve sheath tumors that may cause severe morbidity and possible mortality. The histopathology of these tumors suggests that events connected with formation of fibroblasts might constitute a point of molecular vulnerability. Gene profile analysis demonstrates overexpression of fibroblast growth factor, epidermal growth factor, and platelet-derived growth factor in plexiform neurofibromas in patients with NF1. Formula I-VI is a novel agent that inhibits PAKs.

Objectives:

To determine whether a PAK inhibitor increases the time to disease progression based on volumetric measurements in children and young adults with NF1 and growing plexiform neurofibromas.

To define the objective response rate to a PAK inhibitor in NF1-related plexiform neurofibromas.

To describe and define the toxicities of a PAK inhibitor.

Eligibility:

Individuals (greater than or equal to 3 years to less than or equal to 21 years of age) with a clinical diagnosis of NF1 and inoperable, measurable, and progressive plexiform neurofibromas that have the potential to cause substantial morbidity.

Design:

The phase II dose will be used in a single stage, single arm phase II trial.

Condition Intervention Phase

Neurofibromatosis 1

Neurofibroma, Plexiform

Drug: PAK inhibitor

Phase II

Study Type: Interventional

Study Design: Masking: Open Label

Primary Purpose: Treatment

Official Title: Phase II Trial of a PAK inhibitor in Children, Adolescents, and Young Adults With Neurofibromatosis Type 1 and Progressive Plexiform Neurofibromas

Primary Outcome Measures:

Time to disease progression [Designated as safety issue: No]

Objective response rate [Designated as safety issue: No]

Toxicity [Designated as safety issue: Yes]

Secondary Outcome Measures:

Quality of life [Designated as safety issue: No]

Enrollment: 16

Intervention Details:

Drug: PAK inhibitor

Objectives:

To determine whether a PAK inhibitor increases the time to disease progression based on volumetric measurements in children and young adults with NF1 and growing plexiform neurofibromas.

To define the objective response rate to a PAK inhibitor in NF1-related plexiform neurofibromas.

To describe and define the toxicities of a PAK inhibitor.

Eligibility:

Individuals (greater than or equal to 3 years to less than or equal to 21 years of age) with a clinical diagnosis of NF1 and inoperable, measurable, and progressive plexiform neurofibromas that have the potential to cause substantial morbidity.

Design:

The phase II dose will be used in a single stage, single arm phase II trial The natural history of the growth of plexiform neurofibromas is unknown. For this reason, time to disease progression on the placebo arm of an ongoing NCI POB placebo-controlled, double-blind, cross-over phase II trial of the farnesyltransferase inhibitor R115777 for children and young adults with NF1 and progressive plexiform neurofibromas will be used as historical control to determine if a PAK inhibitor increases time to disease progression. Eligibility criteria and method of tumor measurements are identical for both trials.

A PAK inhibitor will be administered orally as capsules at a dose of 500 mg/m² three times a day (q8h) for cycles of 28 days with no rest period between cycles based on the results of our pediatric phase I trial.

Eligibility

Ages Eligible for Study: 3 Years to 21 Years

Genders Eligible for Study: Both

Accepts Healthy Volunteers: No

Criteria

Inclusion Criteria:

Age: greater than or equal to 3 years and Less than or equal to 21 years of age. Required body surface area (BSA): greater than or equal to 0.31 m².

Diagnosis: Patients with NF1 and progressive plexiform neurofibromas that have the potential to cause significant morbidity, such as (but not limited to) head and neck lesions that could compromise the airway or great vessels, brachial or lumbar plexus lesions that could cause nerve compression and loss of function, lesions that could result in major deformity (e.g., orbital lesions) or significant cosmetic problems, lesions of the extremity that cause limb hypertrophy or loss of function, and painful lesions. Histologic confirmation of tumor is not necessary in the presence of consistent clinical and radiographic findings, but should be considered if malignant degeneration of a plexiform neurofibroma is clinically suspected. In addition to plexiform neurofibroma(s), all study subjects must have at least one other diagnostic criteria for NF1 listed below (NIH Consensus Conference):

Six or more cafe-au-lait spots (greater than or equal to 0.5 cm in prepubertal subjects or greater than or equal to 1.5 cm in postpubertal subjects)

Freckling in the axilla or groin

Optic glioma

Two or more Lisch nodules

A distinctive bony lesion (dysplasia of the sphenoid bone or dysplasia or thinning of long bone cortex)

A first-degree relative with NF1

In this study a plexiform neurofibroma is defined as a neurofibroma that has grown along the length of a nerve and may involve multiple fascicles and branches. A spinal plexiform neurofibroma involves two or more levels with connection between the levels or extending laterally along the nerve.

Measurable disease: Patients must have measurable plexiform neurofibroma(s). For the purpose of this study a measurable lesion will be defined as a lesion of at least 3 cm measured in one dimension. There must be evidence of recurrent or progressive disease as documented by an increase in size or the presence of new plexiform neurofibromas on MRI. Progression at the time of study entry is defined as:

A. A measurable increase of the plexiform neurofibroma (greater than or equal to 20% increase in the volume, or a greater than or equal to 13% increase in the product of the two longest perpendicular diameters, or a greater than or equal to 6% increase in the longest diameter) over the last two consecutive scans (MRI or CT), or over the time period of approximately one year prior to evaluation for this study.

B. Patients who underwent surgery for a progressive plexiform neurofibroma will be eligible to enter the study after the surgery, provided the plexiform neurofibroma was incompletely resected and is measurable.

Prior therapy: Patients with NFI are eligible at the time of recurrence or progression of an inoperable plexiform neurofibroma. Patients will only be eligible if complete tumor resection is not feasible, or if a patient with a surgical option refuses surgery.

Since there is no standard effective chemotherapy for patients with NF1 and progressive plexiform neurofibromas, patients may be treated on this trial without having received prior medical therapy.

Patients who received prior medical treatment for their plexiform neurofibroma(s) must have recovered from the toxic effects of all prior therapy before entering this study. The Cancer Therapy Evaluation Program Common Terminology Criteria (CTCAE-3) Version 3.0 will be used for toxicity assessment. A copy of the CTCAE version 3.0 can be downloaded from the CTEP home page (http://ctep.cancer.gov). Recovery is defined as a toxicity grade less than 2, unless otherwise specified in the

Inclusion and Exclusion Criteria.

Patients must have had their last dose of radiation therapy at least six weeks prior to study entry, and their last dose of chemotherapy at least four weeks prior to study entry. Patients who received G-CSF after the prior cycle of chemotherapy must be off G-CSF for at least one week prior to entering this study.

Performance Status: Performance Status: Patients should have a life expectancy of at least 12 months. Patients greater than 10 years must have a Karnofsky performance level greater than or equal to 50, and children less than or equal to 10 years must have a Lansky performance level greater than or equal to 50. Patients who are wheelchair bound because of paralysis should be considered ambulatory when they are up in their wheel chair.

Hematologic Function: Patients must have an absolute granulocyte count greater than or equal to 1,500/uL, a hemoglobin greater than or equal to 9.0 gm/dl, and a platelet count greater than or equal to 150,000/microliter at study entry (all transfusion independent).

Hepatic Function: Patients must have a bilirubin within normal limits and SGPT less then or equal to 2× upper limit of normal. Patients with Gilbert syndrome are excluded from the requirement of a normal bilirubin. (Gilbert syndrome is found in 3-10% of the general population, and is characterized by mild, chronic unconjugated hyperbilirubinemia in the absence of liver disease or overt hemolysis).

Renal Function: Patients must have an age-adjusted normal serum creatinine (see table below) OR a creatinine clearance greater than or equal to 70 mL/min/1.73 m(2).

| Age Maximum (years) | Serum Creatinine (mg/dl) |
|---|---|
| less than or equal to 5 | 0.8 |
| 5 less than age less than or equal to 10 | 1.0 |
| 10 less than age less than or equal to 15 | 1.2 |
| greater than 15 | 1.5 |

Informed Consent: All patients or their legal guardians (if the patients is less than 18 years old) must sign an IRB approved document of informed consent (screening protocol) prior to performing studies to determine patient eligibility. After confirmation of patient eligibility all patients or their legal guardians must sign the protocol specific informed consent to document their understanding of the investigational nature and the risks of this study before any protocol related studies are performed (other than the studies which were performed to determine patient eligibility). When appropriate, pediatric patients will be included in all discussions. Age appropriate assent forms for children from 7 through 12 years, and for children from 13 through 17 years have been developed and will be signed by the pediatric patients, when appropriate, in order to obtain written assent.

Durable Power of Attorney (DPA): All patients greater than or equal to 18 years of age will be offered the opportunity to assign DPA so that another person can make decisions about their medical care if they become incapacitated or cognitively impaired.

Patients must be able to take PAK inhibitor by mouth. Capsules can be opened and content mixed with food for easier consumption in small children.

Patients (both male and female) must be willing to practice birth control (including abstinence) during and for two months after treatment, if of a child-bearing age. For purposes of the protocol, all patients greater than 9 years of age or those showing pubertal development will be considered of childbearing age.

Ability to undergo MRI and no contraindication for MRI examinations following the MRI protocol outlined.

### Exclusion Criteria:

Pregnant or breast feeding females are excluded, because the toxic effects and pharmacology of a PAK inhibitor in the fetus and newborn are unknown.

Clinically significant unrelated systemic illness (serious infections or significant cardiac, pulmonary, hepatic or other organ dysfunction), which in the judgment of the Principal or Associate Investigator would compromise the patient's ability to tolerate a PAK inhibitor or are likely to interfere with the study procedures or results.

An investigational agent within the past 30 days.

Ongoing radiation therapy, chemotherapy, hormonal therapy directed at the tumor, immunotherapy, or biologic therapy (for example interferon).

Inability to return for follow-up visits or obtain follow-up studies required to assess toxicity and response to therapy.

Evidence of an optic glioma, malignant glioma, malignant peripheral nerve sheath tumor, or other cancer requiring treatment with chemotherapy or radiation therapy

### Example 11

### PAK inhibitors as Monotherapy in the Treatment of Neurofibromatosis Type 2-Related Vestibular Schwannoma

Purpose

The purpose of the study is to determine if a PAK inhibitor treatment will shrink or slow the growth of the vestibular schwannoma(s) in Neurofibromatosis 2 (NF2) patients. Secondary objectives include determining if a PAK inhibitor treatment will improve hearing ability in NF2 patients.

Condition Intervention Phase

Neurofibromatosis Type 2

Neuroma, Acoustic

Drug: PAK inhibitor

Phase II

Study Type: Interventional

Study Design: Endpoint Classification: Efficacy Study

Intervention Model: Single Group Assignment

Masking: Open Label

Primary Purpose: Treatment

Official Title: A Single Arm, Monocenter Phase II Trial of a PAK inhibitor as Monotherapy in the Treatment of Neurofibromatosis Type 2-Related Vestibular Schwannoma

Primary Outcome Measures:

Vestibular schwannoma volume [Time Frame: 1 year (12 months)] [Designated as safety issue: No]

Determine the effect of a PAK inhibitor on change in vestibular schwannoma volume (mm³) by MRI from baseline to 1 year.

Secondary Outcome Measures:

Hearing [Time Frame: 1 year (12 months)] [Designated as safety issue: No]

Determine the effects of a PAK inhibitor treatment on hearing changes (from baseline to 1 year in the ear with the growing vestibular schwannoma.

Number of adverse events [Time Frame: 1 year, 1 month (13 months)] [Designated as safety issue: Yes]

Determine the number of study subjects with adverse events by grade of severity

Estimated Enrollment: 10

Arms Assigned Interventions

PAK inhibitor Treatment: Experimental

All subjects will be given a PAK inhibitor for 1 year (12 months).

Intervention: Drug: PAK inhibitor

Drug: a PAK inhibitor

Adults: 10 mg p.o. daily dose, age 16-17: 3.0 mg/m² p.o. daily

Detailed Description:

This protocol is a Phase II, open-label, efficacy and safety study of single-agent PAK inhibitor in patients with NF2. During the study, subjects will receive continuous daily oral treatment with a PAK inhibitor for up to 1 year or until tumor progression.

Primary Objective: To determine whether a PAK inhibitor has an effect on the VS growth in patients with NF2 at a rate sufficient to submit the drug for further testing.

Secondary Objectives: To determine whether a PAK inhibitor has an effect on the volume of other intracranial tumors, and to assess the effect of a PAK inhibitor on hearing function in patients with NF2 (when applicable).

Eligibility

Ages Eligible for Study: 16 Years to 65 Years

Genders Eligible for Study: Both

Accepts Healthy Volunteers: No

Criteria

Inclusion Criteria:

Diagnosis of NF2 by National Institutes of Health (NIH) criteria

Age≧16 years

Progressive VS growth during the previous 12 months.

WHO performance status > or =2

Adequate bone marrow, liver and renal function.

For women of childbearing potential, no pregnancy or breast-feeding

Willingness and ability to comply with scheduled visits, drug administration plan, laboratory tests, other study procedures, and study restrictions.

Willingness to provide informed consent

Participants with advanced/refractory solid tumors

Exclusion Criteria:

Inability to tolerate periodic MRI scans or gadolinium contrast.

Inability to tolerate periodic audiologic testing or to understand a language with established scoring for word recognition testing.

Inability to adequately perform volumetric measurement of at least 1 target lesion. Note: Patients with cochlear or auditory brainstem implants may participate if a target lesion can be accurately assessed.

Radiation therapy for the target lesion in the 60 months preceding inclusion in the study.

Patients currently receiving anticancer therapies or who have received anticancer therapies within 4 weeks of the start of study drug.

Immunization with attenuated live vaccines within one week of study entry or during study period.

Presence of a fungal infection requiring systemic antifungal treatment at enrollment

Other malignancies within the past 3 years except for adequately treated carcinoma of the cervix or basal or squamous cell carcinomas of the skin.

Patients who have any severe and/or uncontrolled medical conditions.

Patients with a known hypersensitivity to everolimus or other types of rapamycin or to its excipients.

Patients unwilling to or unable to comply with the protocol

### Example 12

### Study to Assess Safety, Pharmacokinetics, and Pharmacodynamics of a PAK inhibitor in Patients With Advanced, Incurable, Solid Tumors in Which the Target Kinases Are Linked to Disease Pathophysiology

Purpose

PAK inhibitors are selective inhibitors of PAK activity. The primary objective of this study is to evaluate the safety and pharmacokinetics of orally administered PAK inhibitor in patients with advanced, incurable, solid tumors in which these target kinases are linked to disease pathophysiology. These tumors include, but are not limited to, acute myelogenous leukemia, gastrointestinal stromal tumor and neurofibromatosis-1, and glioma, breast cancer, prostate cancer, multiple myeloma, Hodgkin lymphoma, melanoma, and osteosarcoma. The secondary objective is to measure the pharmacodynamic activity of a PAK inhibitor via plasma and urine biomarkers of PAK activity.

Condition Intervention Phase

Solid Tumors

Drug: PAK inhibitor

Phase I

Study Type: Interventional

Study Design: Allocation: Non-Randomized

Intervention Model: Single Group Assignment

Masking: Open Label

Primary Purpose: Treatment

Official Title: A Phase 1 Study to Assess Safety, Pharmacokinetics, and Pharmacodynamics of a PAK inhibitor in Patients With Advanced, Incurable, Solid Tumors in Which the Target Kinases Are Linked to Disease Pathophysiology

Primary Outcome Measures:

Safety: subject incidence of adverse events, first-cycle DLTs and clinically significant changes in vital signs, ECGs and clinical laboratory tests [Time Frame: 1 year] [Designated as

safety issue: Yes]

Secondary Outcome Measures:

PK profile: PAK Inhibitor PK parameters including, but not limited to, maximum observed concentration (Cmax), area under the plasma concentration-time curve and half-life [Time Frame: 1 year] [Designated as safety issue: No]

Estimated Enrollment: 24

Arms Assigned Interventions

Intervention: Drug: PAK Inhibitor

Drug: PAK Inhibitor

Capsules administered once or twice daily, continuous dosing

Eligibility

Ages Eligible for Study: 18 Years and older

Genders Eligible for Study: Both

Accepts Healthy Volunteers: No

Criteria

Inclusion Criteria:

Age 18 and older

Solid tumors refractory to standard therapy

ECOG performance status 0 or 1

Life expectancy >3 months

Adequate hepatic, renal, and bone marrow function

Exclusion Criteria:

Specific anti-cancer therapy within 3 weeks of study start

Uncontrolled intercurrent illness

Refractory nausea or vomiting, or malabsorption

Mean QTc>450 msec

### Example 13

### Study of a PAK inhibitor of Recurrent Glioblastoma

Purpose

Primary Objectives

To evaluate the anti-tumor activity of a PAK inhibitor as measured by the 6-month progression free survival (PFS) probability among patients with recurrent Glioblastoma Multiforme (GBM) when administered as monotherapy (cohort A) and in combination with bevacizumab (cohort B).

Secondary Objectives

To evaluate the safety and tolerability of a PAK inhibitor when administered as monotherapy and in combination with bevacizumab among patients with recurrent GBM. To evaluate radiographic response, progression free survival and overall survival of patients with recurrent glioblastoma treated with a PAK inhibitor when administered as monotherapy and in combination with bevacizumab.

This is an open-label phase II study. Two cohorts will accrue and will be assessed sequentially. Each cohort will enroll patients with recurrent GBM. Cohort A will assess recurrent GBM patients who receive a PAK inhibitor monotherapy while Cohort B will assess recurrent GBM patients who receive a PAK inhibitor plus bevacizumab. The primary endpoint of each cohort will be 6-month progression-free survival. For each cohort, a PAK inhibitor will be administered intravenously at 15 mg/kg every week. The dose of bevacizumab will be 10 mg/kg and will be administered intravenously every other week. The estimated rate of accrual is 1-2 patients per month. The estimated date of accrual completion is 5 years from study initiation. The estimated date of study completion will be approximately 12 months from enrollment of the last study patient.

68 subjects will actively participate in this study. In order to accrue 68 actively participating subjects up to 80 subjects may be enrolled.

A PAK inhibitor and bevacizumab will be administered to eligible patients under the supervision of the investigators. The investigators will review all the laboratory data and order the treatment.

For study purposes, a cycle of therapy will be 4 weeks. Treatment will continue until either evidence of progressive disease, unacceptable toxicity, non-compliance with study follow-up, or withdrawal of consent.

Condition Intervention Phase

Glioblastoma Multiforme

Drug: PAK inhibitor

Drug: Bevacizumab

Phase II

Study Type: Interventional

Study Design: Allocation: Non-Randomized

Endpoint Classification: Safety/Efficacy Study

Intervention Model: Parallel Assignment

Masking: Open Label

Primary Purpose: Treatment

Official Title: Phase II Study of a PAK inhibitor of Recurrent Glioblastoma

Primary Outcome Measures:

Radiological response rates [Time Frame: 6 month] [Designated as safety issue: No]

The primary outcome is 6 month progression-free survival. The primary basis for assessing efficacy will be the proportion of patients who survive 6 months without disease progression (PFS-6).

Secondary Outcome Measures:

Radiographic response and Median progression free survival and overall survival. [Time Frame: 6 months] Designated as safety issue: Yes]

Median progression free survival and overall survival. The primary measure of safety outcome will include a tabulation of all grade 2 or greater, treatment related toxicities.

Estimated Enrollment: 68

Arms Assigned Interventions

PAK inhibitor: Experimental

Cohort A will assess recurrent Glioblastoma Multiforme (GBM) patients who receive a PAK inhibitor monotherapy. For each cohort, a PAK inhibitor will be administered intravenously at 15 mg/kg every week.

Intervention: Drug: a PAK inhibitor

Drug: a PAK inhibitor

For each cohort, a PAK inhibitor will be administered intravenously at 15 mg/kg every week.

PAK inhibitor and Bevacizumab: Experimental

Cohort B will assess recurrent Glioblastoma Multiforme (GBM) patients who receive a PAK inhibitor plus bevacizumab. For each cohort, a PAK inhibitor will be administered intravenously at 15 mg/kg every week. The dose of bevacizumab will be 10 mg/kg and will be administered intravenously every other week.

Interventions:

Drug: a PAK inhibitor

Drug: bevacizumab

For each cohort, a PAK inhibitor will be administered intravenously at 15 mg/kg every week.

The dose of bevacizumab will be 10 mg/kg and will be administered intravenously every other week.

Eligibility

Ages Eligible for Study: 18 Years and older

Genders Eligible for Study: Both

Accepts Healthy Volunteers: No

Criteria

Inclusion Criteria:

Patients must have histologically confirmed diagnosis of GBM and radiographic evidence of recurrence or disease progression (defined as either a greater than 25% increase in the largest bidimensional product of enhancement or a new enhancing lesion) following prior therapy (i.e. chemotherapy, XRT, other investigational therapies. In addition, the following must be met:

Age >18 years.

No more than 3 prior episodes of progressive disease;

An interval of at least 4 weeks between prior surgical resection or one week from stereotactic biopsy;

An interval of at least 12 weeks from the end of prior radiotherapy unless there is a new area of enhancement consistent with recurrent tumor outside of the radiation field, or there is histological confirmation of unequivocal tumor progression;

An interval of at least 4 weeks from prior chemotherapy (6 weeks for nitrosoureas) or investigational agent, unless the patient has recovered from all anticipated toxicities associated with that therapy;

Karnofsky at least 70%;

Hematocrit >29%, ANC >1,000 cells/ul, platelets >100,000 cells/ul;

Serum creatinine <1.5 mg/dl, serum SGOT and bilirubin <2.5 times upper limit of normal;

Calculated creatinine clearance >40 mL/min according to the Cockcroft-Gault formula OR per 24 hour urine collection

Signed informed consent approved by the Institutional Review Board prior to patient entry;

No evidence of hemorrhage on the baseline MRI or CT scan other than those that are grade 1 and either post-operative or stable on at least two consecutive scans;

Subjects of child-bearing potential who have not undergone a bilateral salpingo-oopherectomy and are sexually active must consent to use an accepted and effective non-hormonal method of contraception (i.e. double barrier method (e.g., condom plus diaphragm)) from signing the informed consent through 6 months after last dose of study drug.

Exclusion Criteria:

Co-medication that may interfere with study results; e.g. immuno-suppressive agents other than corticosteroids

Active infection requiring intravenous antibiotics

Requires therapeutic anti-coagulation with warfarin

History of arterial or deep venous thromboembolism within 12 months prior to enrollment

History of clinically significant bleeding within 6 months of enrollment

Current or within 30 days prior to enrollment/randomization treatment with immune modulators such as cyclosporine and tacrolimus

History of allergic reactions to bacterially produced proteins

Inability to comply with study and/or follow-up procedures

Current, recent (within 4 weeks of the first infusion of this study), or planned participation in an experimental drug study other than supportive care or epidemiologic studies

Severe hepatic insufficiency (ongoing grade 3 or greater hepatic adverse events) or known active chronic hepatitis

Inadequately controlled hypertension (defined as systolic blood pressure >150 and/or diastolic blood pressure >90 mmHg on antihypertensive medications)

Any prior history of hypertensive crisis or hypertensive encephalopathy

Clinically significant cardiovascular disease within 12 months prior to enrollment (or randomization), including myocardial infarction, unstable angina, grade 2 or greater peripheral vascular disease, cerebrovascular accident, transient ischemic attack, congestive heart failure, or arrhythmias not controlled by outpatient medication, percutaneous transluminal coronary angioplasty/stent

New York Heart Association (NYHA) Grade II or greater congestive heart failure (see Appendix E)

History of myocardial infarction or unstable angina within 6 months prior to study enrollment

History of stroke or transient ischemic attack within 6 months prior to study enrollment

Significant vascular disease (e.g., aortic aneurysm, aortic dissection)

Symptomatic peripheral vascular disease

Evidence of bleeding diathesis or coagulopathy

Major surgical procedure, open biopsy, or significant traumatic injury within 28 days prior to study enrollment or anticipation of need for major surgical procedure during the course of the study

Core biopsy or other minor surgical procedure, excluding placement of a vascular access device, within 7 days prior to study enrollment

History of abdominal fistula, gastrointestinal perforation, or intra-abdominal abscess within 6 months prior to study enrollment

Serious, non-healing wound, ulcer, or bone fracture

Urinary protein quantitative value of <30 mg/dL in urinalysis or <1+ on dipstick, unless quantitative protein is <1000 mg in a 24 hour urine sample

Known hypersensitivity to any component of a PAK inhibitor

Pregnant (positive pregnancy test) or lactating. Refusal or inability to use of effective means of contraception (men and women) in subjects of child-bearing potential

### Example 14

### Safety and Efficacy Study of a PAK inhibitor and Gemcitabine in Combination for Patients with Metastatic or Unresectable Sarcomatoid Renal Cell Carcinoma

Purpose

The goal of this clinical research study is to learn if the combination of 2 drugs (PAK inhibitor and gemcitabine) can help to control metastatic or unresectable renal cell carcinoma. The safety of this drug combination will also be tested.

Objectives:

Primary Objective:

Evaluate progression-free survival with a PAK inhibitor and gemcitabine treatment in metastatic or unresectable renal cell carcinoma (RCC) with sarcomatoid features.

Secondary Objectives:

Evaluate the safety and tolerability of the PAK inhibitor and gemcitabine combination.

Evaluate response rate and overall survival with the PAK inhibitor and gemcitabine combination in metastatic or unresectable RCC with sarcomatoid features.

Develop a prospective archive of tissue and DNA samples from sarcomatoid carcinomas of the kidney.

Condition Intervention Phase

Renal Cell Carcinoma

Kidney Cancer

Drug: PAK inhibitor

Drug: Gemcitabine

Phase II

Study Type: Interventional

Study Design: Allocation: Non-Randomized

Endpoint Classification: Safety/Efficacy Study

Intervention Model: Single Group Assignment

Masking: Open Label

Primary Purpose: Treatment

Official Title: Phase II Safety and Efficacy Study of a PAK inhibitor and Gemcitabine in Combination for Patients with Metastatic or Unresectable Sarcomatoid Renal Cell Carcinoma

Primary Outcome Measures:

Number of Patients with Event Free Survival [Time Frame: Baseline and with each 4 week cycle or until disease progression] [Designated as safety issue: Yes]

Evaluation of response will follow the Response Evaluation Criteria in Solid Tumors (RECIST).

Estimated Enrollment: 40

Arms Assigned Interventions

PAK Inhibitor+Gemcitabine: Experimental

Interventions:

Drug: PAK inhibitor

Drug: Gemcitabine

900 mg/m² By Vein Over 30 Minutes on Days 1 and 15.

Other Names:

Gemzar

Gemcitabine Hydrochloride

Detailed Description:

PAK inhibitors and gemcitabine are designed to disrupt the growth of cancer cells, which may cause cancer cells to start to die. If you are found to be eligible to take part in this study, you will receive a PAK inhibitor and gemcitabine on a 28 day cycle. A PAK inhibitor will be taken by mouth, once daily on days 1-28. Gemcitabine will be given through a needle in your vein in your arm over 30 minutes on Days 1 and 15. On the first day of each cycle, blood (about 2 teaspoons) and a urine will be collected before treatment for routine tests. You will also have blood drawn on Day 15 (about 2 teaspoons) for routine tests.

Every 8 weeks, you will have a CT scan of your chest, abdomen, and pelvis and a chest x-ray. You will be asked about any drugs that you are currently taking and you will have a complete physical exam. You will be asked about any side effects that you might have experienced since the last visit and your ability to perform daily activities will be evaluated. Repeat bone scans and MRI of the brain may be done if your doctor thinks it is necessary.

You will continue receiving treatment for a maximum of 12 months. However, if you are benefiting from treatment, you may be able to continue receiving it off study. You will be taken off study if the disease gets worse, if the side effects are intolerable, or if you develop another illness that prevents you from receiving the treatment.

This is an investigational study. Gemcitabine is FDA approved and commercially available. Up to 40 participants may take part in this study. All will be enrolled at MD Anderson.

Eligibility

Genders Eligible for Study: Both

Accepts Healthy Volunteers: No

Criteria

Inclusion Criteria:

Histologically demonstrated, metastatic or unresectable sarcomatoid carcinoma of the kidney, defined as the following: • A tumor biopsy (primary or metastasis) must show at least one focus of RCC (one of the recognized types); and, • A tumor biopsy (primary or metastasis) must have at least 10% of the sample showing sarcomatoid histology. • Patients with primary tumor in place are eligible if there is any percentage of sarcomatoid dedifferentiation on a needle biopsy (primary or metastasis), and the radiographic appearance of the primary tumor on CT scan is typical of RCC. For these patients, due to the small tumor sample, it is not required to identify an area of typical RCC histology as long as the morphologic and immunostaining characteristics are consistent with RCC.

At least one site of measurable disease (may include primary tumor).

No prior cytotoxic chemotherapy. Any prior immunotherapy is permitted.

Zubrod performance status 2 or better

Adequate organ and bone marrow function: • ANC >/=1,500•Platelets >/=100,000•Total bilirubin </=1.5 mg/dl•AST and ALT </=3× upper limit normal•Creatinine clearance >50 cc/min (measured or calculated by Cockcroft formula: Creatinine Clearance=[(140−age)×wt (kg)]/[72× creat (mg/dl)], for females×0.85. Patients with creatinine clearance of 30-50 ml/min are eligible with an initial dose-reduction of capecitabine to the (−1) dose level.

Female patients of childbearing potential (last menses <2 years) must have a negative blood pregnancy test within 7 days prior to starting treatment.

All patients must agree to practice adequate contraception if sexually active for the duration of the trial and for 2 months after discontinuation of the study drugs

Written informed consent.

Exclusion Criteria:

Patients with history of myocardial infarction, transient ischemic attack (TIA), stroke, pulmonary embolism, or history of deep vein thrombosis within the preceding 12 months.

Patients with major risk of bleeding, such as active brain metastases. Patients with controlled or small brain metastases will be eligible based on clinical assessment of the actual bleeding risk.

Patients with history of any major surgical procedure within the preceding 28 days.

Patients with baseline blood pressure >/=140 systolic or >/=90 diastolic.

Patients with nephrotic syndrome (proteinuria >2 grams per 24 hours)

History of other malignancy, unless it is clinically non-threatening (such as non-melanoma skin cancer) or controlled for 2 years prior to study entry.

Prior treatment with gemcitabine, capecitabine, or any fluoropyrimidine.

Prior unanticipated severe reaction to fluoropyrimidine therapy or known hypersensitivity to 5-FU.

Any concurrent chemotherapy or radiotherapy.

Lack of physical integrity of the upper gastrointestinal tract, inability to swallow tablets or those who have malabsorption syndrome.

Clinically significant cardiac disease not well controlled with medication, such as symptomatic coronary artery disease, congestive heart failure, and cardiac arrhythmias.

Serious concurrent infections or other serious medical conditions, including uncontrolled diabetes.

Any serious non-healing wound, ulcer, or active bone fracture.

Any concurrent coumadin therapy. Patients who were previously on coumadin maintenance may switch to aspirin or low-molecular-weight heparin.

Patients who have had an organ allograft.

Unwillingness to give written informed consent.

### Example 15

### Pharmaceutical Compositions

### Example 15a Parenteral Composition

To prepare a parenteral pharmaceutical composition suitable for administration by injection, 100 mg of a water-soluble salt of a compound of Formula I-VI is dissolved in DMSO and then mixed with 10 mL of 0.9% sterile saline. The mixture is incorporated into a dosage unit form suitable for administration by injection.

### Example 15b

### Oral Composition

To prepare a pharmaceutical composition for oral delivery, 100 mg of a compound of Formula I-VI is mixed with 750 mg of starch. The mixture is incorporated into an oral dosage unit for, e.g., a hard gelatin capsule, which is suitable for oral administration.

### Example 15c

### Sublingual (Hard Lozenge) Composition

To prepare a pharmaceutical composition for buccal delivery, such as a hard lozenge, mix 100 mg of a compound of Formula I-VI with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 15d

### Fast-Disintegrating Sublingual Tablet

A fast-disintegrating sublingual tablet is prepared by mixing 48.5% by weigh of a compound of Formula I-VI, 44.5% by weight of microcrystalline cellulose (KG-802), 5% by weight of low-substituted hydroxypropyl cellulose (50 μm), and 2% by weight of magnesium stearate. Tablets are prepared by direct compression (AAPS PharmSciTech. 2006; 7(2):E41). The total weight of the compressed tablets is maintained at 150 mg. The formulation is prepared by mixing the amount of compound of Formula I-VI with the total quantity of microcrystalline cellulose (MCC) and two-thirds of the quantity of low-substituted hydroxypropyl cellulose (L-HPC) by using a three dimensional manual mixer (Lnversina®, Bioengineering AG, Switzerland) for 4.5 minutes. All of the magnesium stearate (MS) and the remaining one-third of the quantity of L-HPC are added 30 seconds before the end of mixing.

### Example 15e

### Inhalation Composition

To prepare a pharmaceutical composition for inhalation delivery, 20 mg of a compound of Formula I-VI is mixed with 50 mg of anhydrous citric acid and 100 mL of 0.9% sodium chloride solution. The mixture is incorporated into an inhalation delivery unit, such as a nebulizer, which is suitable for inhalation administration.

### Example 15f

### Rectal Gel Composition

To prepare a pharmaceutical composition for rectal delivery, 100 mg of a compound of Formula I-VI is mixed with 2.5 g of methylcellulose (1500 mPa), 100 mg of methylparapen, 5 g of glycerin and 100 mL of purified water. The resulting gel mixture is then incorporated into rectal delivery units, such as syringes, which are suitable for rectal administration.

### Example 15g

### Topical Gel Composition

To prepare a pharmaceutical topical gel composition, 100 mg of a compound of Formula I-VI is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Example 15h

### Ophthalmic Solution Composition

To prepare a pharmaceutical ophthalmic solution composition, 100 mg of a compound of Formula I-VI is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Example 15i

### Nasal Spray Solution

To prepare a pharmaceutical nasal spray solution, 10 g of a compound of Formula I-VI is mixed with 30 mL of a 0.05M phosphate buffer solution (pH 4.4). The solution is placed in a nasal administrator designed to deliver 100 μl of spray for each application.

While some embodiments of the present disclosure have been shown and described herein, such embodiments are provided by way of example only. It is intended that the following claims define the scope of the present disclosure and that methods and structures within the scope of these claims and their equivalents be covered thereby.

What is claimed is:

1. A method for treating a cell proliferative disorder in an individual in need thereof, comprising administering to the subject a therapeutically effective amount of a PAK inhibitor or a pharmaceutically acceptable salt, solvate or N-oxide thereof.

2. The method of claim 1, wherein the cell proliferative disorder is cancer.

3. The method of claim 2, wherein the cancer is a breast cancer, colorectal cancer, brain cancer, lung cancer, pancreatic cancer, kidney cancer, skin cancer, cancer of the central nervous system, liver cancer, stomach cancer, gastrointestinal cancer, ovarian cancer, leukemia, or lymphoma.

4. The method of claim 3, wherein the brain cancer is a glioblastoma.

5. The method of claim 3, wherein the lung cancer is a mesothelioma.

6. The method of claim 3, wherein the cancer of the central nervous system is a tumor associated with neurofibromatosis type 1 or neurofibromatosis type 2.

7. The method of claim 6, wherein the tumor associated with neurofibromatosis type 1 or neurofibromatosis type 2 is a neurofibroma, optic glioma, malignant peripheral nerve sheath tumor, schwannoma, ependymoma, or meningioma.

8. The method of claim 3, wherein the kidney cancer is a renal cell carcinoma.

9. The method of claim 1, wherein the PAK inhibitor is a compound of Formula IV or a pharmaceutically acceptable salt, solvate or N-oxide thereof: 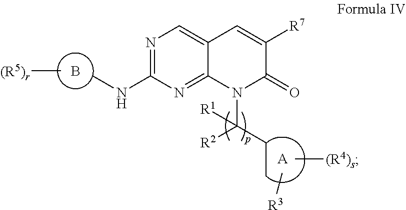
wherein:
R¹ and R² are each independently H or substituted or unsubstituted alkyl; or R¹ and R² together with the carbon to which they are attached form a C₃-C₆ cycloalkyl ring;
p is 1, 2 or 3;
ring A is aryl or heteroaryl;
R³ is S(═O)R⁹ or —S(═O)₂R⁹;
each R⁴ is independently halogen, —CN, —NO₂, —OH, —OCF₃, —OCF₂H, —OCH₂F, —CF₃, —SR⁸, —S(═O)R⁹, —S(═O)₂R⁹, —NR¹⁰S(═O)₂R⁹, —S(═O)₂N(R¹⁰)₂, —OR¹⁰, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, substituted or unsubstituted alkyl, substituted or unsubstituted heteroalkyl, substituted or unsubstituted cycloalkyl or substituted or unsubstituted heterocycloalkyl;
R⁸ is H, substituted or unsubstituted alkyl, substituted or unsubstituted cycloalkyl, substituted or unsubstituted heterocycloalkyl, substituted or unsubstituted aryl or substituted or unsubstituted heteroaryl;
R⁹ is substituted or unsubstituted alkyl, substituted or unsubstituted cycloalkyl, substituted or unsubstituted heterocycloalkyl, substituted or unsubstituted aryl or substituted or unsubstituted heteroaryl;
each R¹⁰ is independently H, substituted or unsubstituted alkyl, substituted or unsubstituted cycloalkyl, substituted or unsubstituted heterocycloalkyl, substituted or unsubstituted aryl or substituted or unsubstituted heteroaryl, or two R¹⁰ together with the atoms to which they are attached form a heterocycle;
s is 0-4;
ring B is aryl or heteroaryl;
each R⁵ is independently halogen, —CN, —NO₂, —OH, —S(═O)R⁹, —S(═O)₂R⁹, NR¹⁰S(═O)₂R⁹, —S(═O)₂N(R¹⁰)₂, —C(═O)R⁹, —OC(═O)R⁹, —CO₂R¹⁰, —N(R¹⁰)₂, —C(═O)N(R¹⁰)₂, —NR¹⁰C(═O)R¹⁰, —NR¹⁰C(═O)OR¹⁰, —NR¹⁰C(═O)N(R¹⁰)₂, —OR¹⁰, substituted or unsubstituted alkyl, substituted or unsubstituted heteroalkyl, substituted or unsubstituted cycloalkyl or substituted or unsubstituted heterocycloalkyl;
r is 0-8;
R⁷ is H, halogen, —CN, substituted or unsubstituted alkyl, —C(═O)N(R¹⁰)₂, —CO₂R¹⁰, —OR¹⁰, —N(R¹⁰)₂, acyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

10. The method of claim 9, wherein R³ is (R)—S(═O)R⁹ or (S)—S(═O)R⁹.

11. The method of claim 9, wherein R³ is —S(═O)₂R⁹.

12. The method of claim 9, wherein R¹ and R² are H; and p is 1.

13. The method of claim 12, wherein ring A is an aryl ring.

14. The method of claim 13, wherein ring A is a phenyl ring.

15. The method of claim 12, wherein ring A is a 5-10-membered heteroaryl ring comprising 0-4 N atoms, 0-2 O atoms, 0-2 S atoms, or any combination thereof; wherein at least one N, S, or O atom is present.

16. The method of claim 15, wherein ring A is selected from pyrrole, furan, thiophene, pyrazole, imidazole, isoxazole, oxazole, isothiazole, thiazole, 1,2,3-triazole, 1,3,4-triazole, 1-oxa-2,3-diazole, 1-oxa-2,4-diazole, 1-oxa-2,5-diazole, 1-oxa-3,4-diazole, 1-thia-2,3-diazole, 1-thia-2,4-diazole, 1-thia-2,5-diazole, 1-thia-3,4-diazole, tetrazole, pyridine, pyridazine, pyrimidine, and pyrazine.

17. The method of claim 9, wherein s is 0.

18. The method of claim 9, wherein ring B is an aryl ring.

19. The method of claim 9, wherein R⁵ is independently)-N(R¹⁰)₂, or substituted or unsubstituted heterocycloalkyl.

20. The method of claim 19, wherein r is 1; and R⁵ is substituted or unsubstituted piperazine.
